(19) United States
(12) Patent Application Publication
(10) Pub. No.: US 2022/0313845 A1
(43) Pub. Date: Oct. 6, 2022

(54) ANTIGEN-BINDING PROTEIN CONSTRUCTS AND USES THEREOF

(71) Applicant: Mythic Therapeutics, Inc., Waltham, MA (US)

(72) Inventors: Alexander J. Nichols, Lincoln, MA (US); Brian P. Fiske, Cambridge, MA (US); Nimish Gera, Waltham, MA (US)

(73) Assignee: Mythic Therapeutics, Inc., Waltham, MA (US)

(21) Appl. No.: 17/616,804

(22) PCT Filed: Jun. 5, 2020
(86) PCT No.: PCT/US2020/036495

§ 371 Date: Dec. 6, 2021

Related U.S. Application Data
(60) Provisional application No. 62/888,384, filed on Aug. 16, 2019.
(60) Provisional application No. 62/858,932, filed on Jun. 7, 2019.

(51) Int. Cl. A61K 51/10; A61K 47/68; A61P 35/00

(52) U.S. Cl. CPC A61K 51/1018; A61K 47/6843; A61P 35/00

(57) ABSTRACT

Provided herein are antigen-binding protein constructs capable of specifically binding DLL3 or an epitope of DLL3 presented on the surface of a target mammalian cell, wherein said antigen binding is pH-dependent. Provided are also uses of said antigen-binding protein constructs.

### CROSS-REFERENCE TO RELATED APPLICATIONS

This application claims priority to U.S. Provisional Patent Application 62/858,932 filed on Jun. 7, 2019 and U.S. Provisional Patent Application 62/888,384 filed on Aug. 16, 2019. The contents of each of these applications are incorporated herein by reference in their entireties.

### TECHNICAL FIELD

The present disclosure relates to the field of biotechnology, and more specifically, to antigen-binding molecules.

### BACKGROUND

Antibody-drug-conjugates have been designed to combat a variety of diseases. One particular advantage of this approach is the ability for antibody-drug conjugates to have cytostatic or cytotoxic effects. Despite years of development, improved antibody-drug conjugates are desired.

### SUMMARY

The present invention is based on the concept that antigen-binding protein constructs can be generated that display enhanced efficacy (e.g., one or more of an increase (e.g., a detectable increase) in toxin liberation in a target mammalian cell, an increase (e.g., a detectable increase) in target mammalian cell killing, and an increase (e.g., a detectable increase) in endolysosomal delivery).

Provided herein are pharmaceutical compositions including an effective amount of an antigen-binding protein construct (ABPC) including a first antigen-binding domain that is capable of specifically binding DLL3 or an epitope of DLL3 presented on the surface of a target mammalian cell, where (a) the dissociation rate of the first antigen-binding domain at a pH of about 4.0 to about 6.5 is faster than the dissociation rate at a pH of about 7.0 to about 8.0; or (b) the dissociation constant (KD) of the first antigen-binding domain at a pH of about 4.0 to about 6.5 is greater than the KD at a pH of about 7.0 to about 8.0.

In some embodiments of any of the pharmaceutical compositions described herein, the ABPC is degraded in the target mammalian cell following internalization of the ABPC by the target mammalian cell.

In some embodiments of any of the pharmaceutical compositions described herein, the ABPC includes a conjugated toxin, radioisotope, drug, or small molecule.

Also provided herein are pharmaceutical compositions including an effective amount of an antigen-binding protein construct (ABPC) including, a first antigen-binding domain that is capable of specifically binding DLL3 or an epitope of DLL3 presented on the surface of a target mammalian cell and a conjugated toxin, radioisotope, drug, or small molecule, where (a) the dissociation rate of the first antigen-binding domain at a pH of about 4.0 to about 6.5 is faster than the dissociation rate at a pH of about 7.0 to about 8.0 or the dissociation constant (KD) of the first antigen-binding domain at a pH of about 4.0 to about 6.5 is greater than the KD at a pH of about 7.0 to about 8.0 and (b) the composition provides for one or more of an increase in toxin liberation in the target mammalian cell as compared to a composition including the same amount of a control ABPC an increase in target mammalian cell killing as compared to a composition including the same amount of a control ABPC and an increase in endolysosomal delivery in the target mammalian cell as compared to a composition including the same amount of a control ABPC.

In some embodiments of any of the pharmaceutical compositions described herein, the first antigen-binding domain includes one of (a) though (g): (a) a heavy chain variable domain of rovalpituzumab with one or more amino acids substituted with a histidine, where the heavy chain variable domain of rovalpituzumab includes SEQ ID NO: 1; and/or a light chain variable domain of rovalpituzumab with one or more amino acids substituted with a histidine, where the light chain variable domain of rovalpituzumab includes SEQ ID NO: 2; (b) a heavy chain variable domain of SC16.4 with one or more amino acids substituted with a histidine, where the heavy chain variable domain of SC16.4 includes SEQ ID NO: 119; and/or a light chain variable domain of SC16.4 with one or more amino acids substituted with a histidine, where the light chain variable domain of SC16.4 includes SEQ ID NO: 120; (c) a heavy chain variable domain of SC16.13 with one or more amino acids substituted with a histidine, where the heavy chain variable domain of SC16.13 includes SEQ ID NO: 236; and/or a light chain variable domain of SC16.13 with one or more amino acids substituted with a histidine, where the light chain variable domain of SC16.13 includes SEQ ID NO: 237; (d) a heavy chain variable domain of SC16.15 with one or more amino acids substituted with a histidine, where the heavy chain variable domain of SC16.15 includes SEQ ID NO: 335; and/or a light chain variable domain of SC16.15 with one or more amino acids substituted with a histidine, where the light chain variable domain of SC16.15 includes SEQ ID NO: 336; (e) a heavy chain variable domain of SC16.25 with one or more amino acids substituted with a histidine, where the heavy chain variable domain of SC16.25 includes SEQ ID NO: 401; and/or a light chain variable domain of SC16.25 with one or more amino acids substituted with a histidine, where the light chain variable domain of SC16.25 includes SEQ ID NO: 402; (f) a heavy chain variable domain of SC16.34 with one or more amino acids substituted with a histidine, where the heavy chain variable domain of SC16.34 includes SEQ ID NO: 470; and/or a light chain variable domain of SC16.34 with one or more amino acids substituted with a histidine, where the light chain variable domain of SC16.34 includes SEQ ID NO: 471; and (g) a heavy chain variable domain of SC16.67 with one or more amino acids substituted with a histidine, where the heavy chain variable domain of SC16.67 includes SEQ ID NO: 537; and/or a light chain variable domain of SC16.67 with one or more amino acids substituted with a histidine, where the light chain variable domain of SC16.67 includes SEQ ID NO: 538.

In some embodiments of any of the pharmaceutical compositions described herein, the first DLL3-binding domain includes one of (a) through (g): (a) a heavy chain variable domain including a CDR1, a CDR2, and a CDR3 of SEQ ID NOs: 3-5, respectively, with collectively a total of one or more amino acid positions in SEQ ID NOs: 3-5 substituted with a histidine; and/or a light chain variable domain including a CDR1, a CDR2, and a CDR3 of SEQ ID NOs: 6-8, respectively, with collectively a total of one or more amino acid positions in SEQ ID NOs: 6-8 substituted with a histidine; (b) a heavy chain variable domain including a CDR1, a CDR2, and a CDR3 of SEQ ID NOs: 622-624, respectively, with collectively a total of one or more amino acid positions in SEQ ID NOs: 622-624 substituted with a histidine; and/or a light chain variable domain including a CDR1, a CDR2, and a CDR3 of SEQ ID NOs: 625-627, respectively, with collectively a total of one or more amino acid positions in SEQ ID NOs: 625-627 substituted with a histidine; (c) a heavy chain variable domain including a CDR1, a CDR2, and a CDR3 of SEQ ID NOs: 628-630, respectively, with collectively a total of one or more amino acid positions in SEQ ID NOs: 628-630 substituted with a histidine; and/or a light chain variable domain including a CDR1, a CDR2, and a CDR3 of SEQ ID NOs: 631-633, respectively, with collectively a total of one or more amino acid positions in SEQ ID NOs: 631-633 substituted with a histidine; (d) a heavy chain variable domain including a CDR1, a CDR2, and a CDR3 of SEQ ID NOs: 634-636, respectively, with collectively a total of one or more amino acid positions in SEQ ID NOs: 634-636 substituted with a histidine; and/or a light chain variable domain including a CDR1, a CDR2, and a CDR3 of SEQ ID NOs: 637-639, respectively, with collectively a total of one or more amino acid positions in SEQ ID NOs: 637-639 substituted with a histidine; (e) a heavy chain variable domain including a CDR1, a CDR2, and a CDR3 of SEQ ID NOs: 640-642, respectively, with collectively a total of one or more amino acid positions in SEQ ID NOs: 640-642 substituted with a histidine; and/or a light chain variable domain including a CDR1, a CDR2, and a CDR3 of SEQ ID NOs: 643-645, respectively, with collectively a total of one or more amino acid positions in SEQ ID NOs: 643-645 substituted with a histidine; (f) a heavy chain variable domain including a CDR1, a CDR2, and a CDR3 of SEQ ID NOs: 646-648, respectively, with collectively a total of one or more amino acid positions in SEQ ID NOs: 646-648 substituted with a histidine; and/or a light chain variable domain including a CDR1, a CDR2, and a CDR3 of SEQ ID NOs: 649-651, respectively, with collectively a total of one or more amino acid positions in SEQ ID NOs: 649-651 substituted with a histidine; and (g) a heavy chain variable domain including a CDR1, a CDR2, and a CDR3 of SEQ ID NOs: 652-654, respectively, with collectively a total of one or more amino acid positions in SEQ ID NOs: 652-654 substituted with a histidine; and/or a light chain variable domain including a CDR1, a CDR2, and a CDR3 of SEQ ID NOs: 655-657, respectively, with collectively a total of one or more amino acid positions in SEQ ID NOs: 655-657 substituted with a histidine.

In some embodiments of any of the pharmaceutical compositions described herein, the first antigen-binding domain includes one of (a) through (g): (a) a heavy chain variable domain that is at least 90% identical to SEQ ID NO: 1, where the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and/or a light chain variable domain that is at least 90% identical to SEQ ID NO: 2, where the light chain variable domain includes a histidine at one or more positions in SEQ ID NO: 2 selected from the group consisting of: 25, 26, 29, 32, 33, 34, 51, 54, 89, 90, 93, 94, 95, and 96; (b) a heavy chain variable domain that is at least 90% identical to SEQ ID NO: 119, where the heavy chain variable domain includes a histidine at one or more positions in SEQ ID NO: 119 selected from the group consisting of: 32, 50, 52, 53, 57, 58, 59, 60, 62, 64, 65, 97, 98, 99, 100, 102, 104, and 105; and/or a light chain variable domain that is at least 90% identical to SEQ ID NO: 120, where the light chain variable domain includes a histidine at one or more positions in SEQ ID NO: 120 selected from the group consisting of: 29, 32, 34, 91, 92, 93, 94 and 96; (c) a heavy chain variable domain that is at least 90% identical to SEQ ID NO: 236, where the heavy chain variable domain includes a histidine at one or more positions in SEQ ID NO: 236 selected from the group consisting of: 27, 29, 31, 33, 34, 35, 36, 37, 54, 55, 56, 58, 60, 98, 100, 102, 106, 108, and 110; and/or a light chain variable domain that is at least 90% identical to SEQ ID NO: 237, where the light chain variable domain includes a histidine at one or more positions in SEQ ID NO: 237 selected from the group consisting of 25, 29, 32, 33, 49, 50, 90, 91, 93, and 95; (d) a heavy chain variable domain that is at least 90% identical to SEQ ID NO: 335, where the heavy chain variable domain includes a histidine at one or more positions in SEQ ID NO: 335 selected from the group consisting of: 27, 32, 33, 34, 50, 98, 101, 102, 103, 104, and 105; and/or a light chain variable domain that is at least 90% identical to SEQ ID NO: 336, where the light chain variable domain includes a histidine at one or more positions in SEQ ID NO: 336 selected from the group consisting of: 27, 29, 32, 34, 52, 53, 89, 90, 91, 92, 93, 94, and 96; (e) a heavy chain variable domain that is at least 90% identical to SEQ ID NO: 401, where the heavy chain variable domain includes a histidine at one or more positions in SEQ ID NO: 401 selected from the group consisting of: 27, 101, 103, 104, 108, and 109; and/or a light chain variable domain that is at least 90% identical to SEQ ID NO: 402, where the light chain variable domain includes a histidine at one or more positions in SEQ ID NO: 402 selected from the group consisting of: 30 and 31; (f) a heavy chain variable domain that is at least 90% identical to SEQ ID NO: 470, where the heavy chain variable domain includes a histidine at one or more positions in SEQ ID NO: 470 selected from the group consisting of 24, 27, 29, 32, 34, 35, 50, 51, 53, 54, 56, 58, 59, 60, 63, 98, 101, 103, 105, 106, and 107; and/or a light chain variable domain that is at least 90% identical to SEQ ID NO: 471, where the light chain variable domain includes a histidine at one or more positions in SEQ ID NO: 471 selected from the group consisting of: 29, 32, 33, 34, 50, 51, 53, 55, 89, 92, 94, 96 and 97; and (g) a heavy chain variable domain that is at least 90% identical to SEQ ID NO: 537, where the heavy chain variable domain includes a histidine at one or more positions in SEQ ID NO: 537 selected from the group consisting of: 26, 29, 31, 32, 53, 54, 57, 58, 59, 65, 67, 68, 103, 104, and 106; and/or a light chain variable domain that is at least 90% identical to SEQ ID NO: 538, where the light chain variable domain includes a histidine at one or more positions in SEQ ID NO: 538 selected from the group consisting of: 28, 34, 53, 93, 94, and 98.

In some embodiments of any of the pharmaceutical compositions described herein, the first antigen-binding domain includes one of (a) through (g): (a) a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 65, SEQ ID NO: 66, SEQ ID NO: 69, SEQ ID NO: 72, SEQ ID NO: 73, SEQ ID NO: 76, SEQ ID NO: 77, SEQ ID NO: 78, SEQ ID NO: 79, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, or SEQ ID NO: 118, and/or a heavy chain variable domain of SEQ ID NO: 1, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, SEQ ID NO: 52, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98, where the first antigen-binding domain does not comprise (i) a light chain variable domain of SEQ ID NO: 2 and a heavy chain variable domain of SEQ ID NO: 1; (ii) a light chain variable domain of SEQ ID NO: 2 and heavy chain variable domain that is not one of SEQ ID NOs: 17, 19, 21, 22, 24-26, 29-31, 34, 43, 44, 47, 49, 51, 52, and 81-98; or (iii) a heavy chain variable domain of SEQ ID NO: 1 and a light chain variable domain that is not one of SEQ ID NOs: 55, 56, 59, 62-66, 69, 72, 73, 76-79, and 99-118; (b) a light chain variable domain of SEQ ID NO: 120, SEQ ID NO: 163, SEQ ID NO: 166, SEQ ID NO: 168, SEQ ID NO: 178, SEQ ID NO: 179, SEQ ID NO: 180, SEQ ID NO: 181, SEQ ID NO: 183, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, or SEQ ID NO: 235, and/or a heavy chain variable domain of SEQ ID NO: 119, SEQ ID NO: 127, SEQ ID NO: 130, SEQ ID NO: 131, SEQ ID NO: 133, SEQ ID NO: 134, SEQ ID NO: 138, SEQ ID NO: 139, SEQ ID NO: 140, SEQ ID NO: 141, SEQ ID NO: 143, SEQ ID NO: 145, SEQ ID NO: 146, SEQ ID NO: 148, SEQ ID NO: 149, SEQ ID NO: 150, SEQ ID NO: 151, SEQ ID NO: 153, SEQ ID NO: 155, SEQ ID NO: 156; SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 216, SEQ ID NO: 217, SEQ ID NO: 218, SEQ ID NO: 220, SEQ ID NO: 221, or SEQ ID NO: 223, where the first antigen-binding domain does not comprise (i) a light chain variable domain of SEQ ID NO: 120 and a heavy chain variable domain of SEQ ID NO: 119; (ii) a light chain variable domain of SEQ ID NO: 120 and heavy chain variable domain that is not one of SEQ ID NOs: 127, 130, 131, 133, 134, 138-141, 143, 145, 146, 148-151, 153, 155, 156, 185-214, 216-218, 220, 221, and 223; or (iii) a heavy chain variable domain of SEQ ID NO: 119 and a light chain variable domain that is not one of SEQ ID NOs: 163, 166, 168, 178-181, 183, and 226-235; (c) a light chain variable domain of SEQ ID NO: 237, SEQ ID NO: 283, SEQ ID NO: 287, SEQ ID NO: 290, SEQ ID NO: 291, SEQ ID NO: 292, SEQ ID NO: 293, SEQ ID NO: 301, SEQ ID NO: 302, SEQ ID NO: 304, SEQ ID NO: 306, or SEQ ID NO: 334, and/or a heavy chain variable domain of SEQ ID NO: 236, SEQ ID NO: 239, SEQ ID NO: 241, SEQ ID NO: 243, SEQ ID NO: 245, SEQ ID NO: 246, SEQ ID NO: 247, SEQ ID NO: 248, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 268, SEQ ID NO: 270, SEQ ID NO: 274, SEQ ID NO: 276, SEQ ID NO: 278, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 330, SEQ ID NO: 331, or SEQ ID NO: 332, where the first antigen-binding domain does not comprise (i) a light chain variable domain of SEQ ID NO: 237 and a heavy chain variable domain of SEQ ID NO: 236; (ii) a light chain variable domain of SEQ ID NO: 237 and heavy chain variable domain that is not one of SEQ ID NOs: 239, 241, 243, 245-250, 252-254, 256, 258, 266, 268, 270, 274, 276, 278, 308-328, and 330-332; or (iii) a heavy chain variable domain of SEQ ID NO: 236 and a light chain variable domain that is not one of SEQ ID NOs: 283, 287, 290-293, 301, 302, 304, 306, and 334; (d) a light chain variable domain of SEQ ID NO: 336, SEQ ID NO: 377, SEQ ID NO: 379, SEQ ID NO: 382, SEQ ID NO: 384, SEQ ID NO: 387, SEQ ID NO: 388, SEQ ID NO: 392, SEQ ID NO: 393, SEQ ID NO: 394, SEQ ID NO: 395, SEQ ID NO: 396, SEQ ID NO: 397, or SEQ ID NO: 399, and/or a heavy chain variable domain of SEQ ID NO: 335, SEQ ID NO: 341, SEQ ID NO: 346, SEQ ID NO: 347, SEQ ID NO: 348, SEQ ID NO: 349, SEQ ID NO: 350, SEQ ID NO: 368, SEQ ID NO: 371, SEQ ID NO: 372, SEQ ID NO: 373, SEQ ID NO: 374, or SEQ ID NO: 375, where the first antigen-binding domain does not comprise (i) a light chain variable domain of SEQ ID NO: 336 and a heavy chain variable domain of SEQ ID NO: 335; (ii) a light chain variable domain of SEQ ID NO: 336 and heavy chain variable domain that is not one of SEQ ID NOs: 341, 346-350, 368, and 371-375; or (iii) a heavy chain variable domain of SEQ ID NO: 335 and a light chain variable domain that is not one of SEQ ID NOs: 377, 379, 382, 384, 387, 388, 392-397, and 399; (e) a light chain variable domain of SEQ ID NO: 402, SEQ ID NO: 450, or SEQ ID NO: 451, and/or a heavy chain variable domain of SEQ ID NO: 401, SEQ ID NO: 404, SEQ ID NO: 434, SEQ ID NO: 436, SEQ ID NO: 437, SEQ ID NO: 441, or SEQ ID NO: 442, where the first antigen-binding domain does not comprise (i) a light chain variable domain of SEQ ID NO: 402 and a heavy chain variable domain of SEQ ID NO: 401; (ii) a light chain variable domain of SEQ ID NO: 402 and heavy chain variable domain that is not one of SEQ ID NOs: 404, 434, 436, 437, 441, and 442; or (iii) a heavy chain variable domain of SEQ ID NO: 401 and a light chain variable domain that is not one of SEQ ID NO: 450 or 451; and/or (f) a light chain variable domain of SEQ ID NO: 471, SEQ ID NO: 515, SEQ ID NO: 518, SEQ ID NO: 519, SEQ ID NO: 520, SEQ ID NO: 521, SEQ ID NO: 522, SEQ ID NO: 524, SEQ ID NO: 526, SEQ ID NO: 528, SEQ ID NO: 531, SEQ ID NO: 533, SEQ ID NO: 535, or SEQ ID NO: 536, and/or a heavy chain variable domain of SEQ ID NO: 470, SEQ ID NO: 473, SEQ ID NO: 476, SEQ ID NO: 478, SEQ ID NO: 481, SEQ ID NO: 483, SEQ ID NO: 484, SEQ ID NO: 485, SEQ ID NO: 486, SEQ ID NO: 488, SEQ ID NO: 489, SEQ ID NO: 491, SEQ ID NO: 493, SEQ ID NO: 494, SEQ ID NO: 495, SEQ ID NO: 498, SEQ ID NO: 503, SEQ ID NO: 506, SEQ ID NO: 508, SEQ ID NO: 510, SEQ ID NO: 511, or SEQ ID NO: 512, where the first antigen-binding domain does not comprise (i) a light chain variable domain of SEQ ID NO: 471 and a heavy chain variable domain of SEQ ID NO: 470; (ii) a light chain variable domain of SEQ ID NO: 471 and heavy chain variable domain that is not one of SEQ ID NOs: 473, 476, 478, 481, 483-486, 488, 489, 491, 493-495, 498, 503, 506, 508, and 510-512; or (iii) a heavy chain variable domain of SEQ ID NO: 470 and a light chain variable domain that is not one of SEQ ID NOs: 515, 518-522, 524, 526, 528, 531, 533, 535, and 536; and (g) a light chain variable domain of SEQ ID NO: 538, SEQ ID NO: 581, SEQ ID NO: 587, SEQ ID NO: 591, SEQ ID NO: 599, SEQ ID NO: 600, or SEQ ID NO: 604, and/or a heavy chain variable domain of SEQ ID NO: 537, SEQ ID NO: 539, SEQ ID NO: 542, SEQ ID NO: 544, SEQ ID NO: 545, SEQ ID NO: 552, SEQ ID NO: 553, SEQ ID NO: 556, SEQ ID NO: 557, SEQ ID NO: 558, SEQ ID NO: 564, SEQ ID NO: 566, SEQ ID NO: 567, SEQ ID NO: 572, SEQ ID NO: 573, or SEQ ID NO: 575, where the first antigen-binding domain does not comprise (i) a light chain variable domain of SEQ ID NO: 538 and a heavy chain variable domain of SEQ ID NO: 537; (ii) a light chain variable domain of SEQ ID NO: 538 and heavy chain variable domain that is not one of SEQ ID NOs: 539, 542, 544, 545, 552, 553, 556-558, 564, 566, 567, 572, 573, and 575; or (iii) a heavy chain variable domain of SEQ ID NO: 537 and a light chain variable domain that is not one of SEQ ID NOs: 581, 587, 591, 599, 600, and 604.

In some embodiments of any of the pharmaceutical compositions described herein, the composition provides for an increase in toxin liberation in the target mammalian cell as compared to a composition including the same amount of a control ABPC and/or an increase in target mammalian cell killing as compared to a composition including the same amount of a control ABPC.

In some embodiments of any of the pharmaceutical compositions described herein, the composition provides for an increase in endolysosomal delivery in the target mammalian cell as compared to a composition including the same amount of a control ABPC.

In some embodiments of any of the pharmaceutical compositions described herein, the composition results in a less of a reduction in the level of DLL3 presented on the surface of the target mammalian cell as compared to a composition including the same amount of a control ABPC or does not result in a detectable reduction in the level of DLL3 presented on the surface of the target mammalian cell.

In some embodiments of any of the pharmaceutical compositions described herein, the target mammalian cell is a cancer cell. In some embodiments of the pharmaceutical compositions described herein, the ABPC is cytotoxic or cytostatic to the target mammalian cell.

In some embodiments of any of the pharmaceutical compositions described herein, the ABPC is cross-reactive with a non-human primate DLL3 and human DLL3 or cross-reactive with a non-human primate DLL3, a human DLL3, and one or both of rat DLL3 and a mouse DLL3.

In some embodiments of any of the pharmaceutical compositions described herein, the ABPC includes a single polypeptide. In some embodiments of any of the pharmaceutical compositions described herein, the antigen-binding domain is selected from the group consisting of: a VH domain, a VHH domain, a VNAR domain, and a scFv. In some embodiments of any of the pharmaceutical compositions described herein, the ABPC includes two or more polypeptides. In some embodiments of any of the pharmaceutical compositions described herein, the ABPC is an antibody.

In some embodiments of any of the pharmaceutical compositions described herein, the half-life of the ABPC in vivo is decreased as compared to the half-life of a control ABPC in vivo.

In some embodiments of any of the pharmaceutical compositions described herein, the ABPC includes a second antigen-binding domain.

Also provided herein are an antigen-binding protein constructs (ABPC) including: a first antigen-binding domain that is capable of specifically binding DLL3 or an epitope of DLL3 presented on the surface of a target mammalian cell, where (a) the dissociation rate of the first antigen-binding domain at a pH of about 4.0 to about 6.5 is faster than the dissociation rate at a pH of about 7.0 to about 8.0 or (b) the dissociation constant (KD) of the first antigen-binding domain at a pH of about 4.0 to about 6.5 is greater than the KD at a pH of about 7.0 to about 8.0.

In some embodiments of any of the ABPCs described herein, the ABPC is degraded in the target mammalian cell following internalization of the ABPC by the target mammalian cell.

In some embodiments of any of the ABPCs described herein the ABPC includes a conjugated toxin, radioisotope, drug, or small molecule.

Also provided herein are an antigen-binding protein constructs (ABPC) including a first antigen-binding domain that is capable of specifically binding DLL3 or an epitope of DLL3 presented on the surface of a target mammalian cell and a conjugated toxin, radioisotope, drug, or small molecule, where (a) the dissociation rate of the first antigen-binding domain at a pH of about 4.0 to about 6.5 is faster than the dissociation rate at a pH of about 7.0 to about 8.0; or the dissociation constant (KD) of the first antigen-binding domain at a pH of about 4.0 to about 6.5 is greater than the KD at a pH of about 7.0 to about 8.0 and (b) the composition provides for one or more of an increase in toxin liberation in the target mammalian cell as compared to a composition including the same amount of a control ABPC, an increase in target mammalian cell killing as compared to a composition including the same amount of a control ABPC, and an increase in endolysosomal delivery in the target mammalian cell as compared to a composition including the same amount of a control ABPC.

In some embodiments of any of the ABPCs described herein the first antigen-binding domain includes one of (a) through (g): (a) a heavy chain variable domain of rovalpituzumab with one or more amino acids substituted with a histidine, where the heavy chain variable domain of rovalpituzumab includes SEQ ID NO: 1; and/or a light chain variable domain of rovalpituzumab with one or more amino acids substituted with a histidine, where the light chain variable domain of rovalpituzumab includes SEQ ID NO: 2; (b) a heavy chain variable domain of SC16.4 with one or more amino acids substituted with a histidine, where the heavy chain variable domain of SC16.4 includes SEQ ID NO: 119; and/or a light chain variable domain of SC16.4 with one or more amino acids substituted with a histidine, where the light chain variable domain of SC16.4 includes SEQ ID NO: 120; (c) a heavy chain variable domain of SC16.13 with one or more amino acids substituted with a histidine, where the heavy chain variable domain of SC16.13 includes SEQ ID NO: 236; and/or a light chain variable domain of SC16.13 with one or more amino acids substituted with a histidine, where the light chain variable domain of SC16.13 includes SEQ ID NO: 237; (d) a heavy chain variable domain of SC16.15 with one or more amino acids substituted with a histidine, where the heavy chain variable domain of SC16.15 includes SEQ ID NO: 335; and/or a light chain variable domain of SC16.15 with one or more amino acids substituted with a histidine, where the light chain variable domain of SC16.15 includes SEQ ID NO: 336; (e) a heavy chain variable domain of SC16.25 with one or more amino acids substituted with a histidine, where the heavy chain variable domain of SC16.25 includes SEQ ID NO: 401; and/or a light chain variable domain of SC16.25 with one or more amino acids substituted with a histidine, where the light chain variable domain of SC16.25 includes SEQ ID NO: 402; (f) a heavy chain variable domain of SC16.34 with one or more amino acids substituted with a histidine, where the heavy chain variable domain of SC16.34 includes SEQ ID NO: 470; and/or a light chain variable domain of SC16.34 with one or more amino acids substituted with a histidine, where the light chain variable domain of SC16.34 includes SEQ ID NO: 471; and (g) a heavy chain variable domain of SC16.67 with one or more amino acids substituted with a histidine, where the heavy chain variable domain of SC16.67 includes SEQ ID NO: 537; and/or a light chain variable domain of SC16.67 with one or more amino acids substituted with a histidine, where the light chain variable domain of SC16.67 includes SEQ ID NO: 538.

In some embodiments of any of the ABPCs described herein the first DLL3-binding domain includes one of (a) through (g): (a) a heavy chain variable domain including a CDR1, a CDR2, and a CDR3 of SEQ ID NOs: 3-5, respectively, with collectively a total of one or more amino acid positions in SEQ ID NOs: 3-5 substituted with a histidine; and/or a light chain variable domain including a CDR1, a CDR2, and a CDR3 of SEQ ID NOs: 6-8, respectively, with collectively a total of one or more amino acid positions in SEQ ID NOs: 6-8 substituted with a histidine; (b) a heavy chain variable domain including a CDR1, a CDR2, and a CDR3 of SEQ ID NOs: 622-624, respectively, with collectively a total of one or more amino acid positions in SEQ ID NOs: 622-624 substituted with a histidine; and/or a light chain variable domain including a CDR1, a CDR2, and a CDR3 of SEQ ID NOs: 625-627, respectively, with collectively a total of one or more amino acid positions in SEQ ID NOs: 625-627 substituted with a histidine; (c) a heavy chain variable domain including a CDR1, a CDR2, and a CDR3 of SEQ ID NOs: 628-630, respectively, with collectively a total of one or more amino acid positions in SEQ ID NOs: 628-630 substituted with a histidine; and/or a light chain variable domain including a CDR1, a CDR2, and a CDR3 of SEQ ID NOs: 631-633, respectively, with collectively a total of one or more amino acid positions in SEQ ID NOs: 631-633 substituted with a histidine; (d) a heavy chain variable domain including a CDR1, a CDR2, and a CDR3 of SEQ ID NOs: 634-636, respectively, with collectively a total of one or more amino acid positions in SEQ ID NOs: 634-636 substituted with a histidine; and/or a light chain variable domain including a CDR1, a CDR2, and a CDR3 of SEQ ID NOs: 637-639, respectively, with collectively a total of one or more amino acid positions in SEQ ID NOs: 637-639 substituted with a histidine; (e) a heavy chain variable domain including a CDR1, a CDR2, and a CDR3 of SEQ ID NOs: 640-642, respectively, with collectively a total of one or more amino acid positions in SEQ ID NOs: 640-642 substituted with a histidine; and/or a light chain variable domain including a CDR1, a CDR2, and a CDR3 of SEQ ID NOs: 643-645, respectively, with collectively a total of one or more amino acid positions in SEQ ID NOs: 643-645 substituted with a histidine; (f) a heavy chain variable domain including a CDR1, a CDR2, and a CDR3 of SEQ ID NOs: 646-648, respectively, with collectively a total of one or more amino acid positions in SEQ ID NOs: 646-648 substituted with a histidine; and/or a light chain variable domain including a CDR1, a CDR2, and a CDR3 of SEQ ID NOs: 649-651, respectively, with collectively a total of one or more amino acid positions in SEQ ID NOs: 649-651 substituted with a histidine; and (g) a heavy chain variable domain including a CDR1, a CDR2, and a CDR3 of SEQ ID NOs: 652-654, respectively, with collectively a total of one or more amino acid positions in SEQ ID NOs: 652-654 substituted with a histidine; and/or a light chain variable domain including a CDR1, a CDR2, and a CDR3 of SEQ ID NOs: 655-657, respectively, with collectively a total of one or more amino acid positions in SEQ ID NOs: 655-657 substituted with a histidine.

In some embodiments of any of the ABPCs described herein, the first antigen-binding domain includes one of (a) through (g): (a) a heavy chain variable domain that is at least 90% identical to SEQ ID NO: 1, where the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and/or a light chain variable domain that is at least 90% identical to SEQ ID NO: 2, where the light chain variable domain includes a histidine at one or more positions in SEQ ID NO: 2 selected from the group consisting of: 25, 26, 29, 32, 33, 34, 51, 54, 89, 90, 93, 94, 95, and 96; (b) a heavy chain variable domain that is at least 90% identical to SEQ ID NO: 119, where the heavy chain variable domain includes a histidine at one or more positions in SEQ ID NO: 119 selected from the group consisting of: 32, 50, 52, 53, 57, 58, 59, 60, 62, 64, 65, 97, 98, 99, 100, 102, 104, and 105; and/or a light chain variable domain that is at least 90% identical to SEQ ID NO: 120, where the light chain variable domain includes a histidine at one or more positions in SEQ ID NO: 120 selected from the group consisting of: 29, 32, 34, 91, 92, 93, 94 and 96; (c) a heavy chain variable domain that is at least 90% identical to SEQ ID NO: 236, where the heavy chain variable domain includes a histidine at one or more positions in SEQ ID NO: 236 selected from the group consisting of: 27, 29, 31, 33, 34, 35, 36, 37, 54, 55, 56, 58, 60, 98, 100, 102, 106, 108, and 110; and/or a light chain variable domain that is at least 90% identical to SEQ ID NO: 237, where the light chain variable domain includes a histidine at one or more positions in SEQ ID NO: 237 selected from the group consisting of: 25, 29, 32, 33, 49, 50, 90, 91, 93, and 95; (d) a heavy chain variable domain that is at least 90% identical to SEQ ID NO: 335, where the heavy chain variable domain includes a histidine at one or more positions in SEQ ID NO: 335 selected from the group consisting of 27, 32, 33, 34, 50, 98, 101, 102, 103, 104, and 105; and/or a light chain variable domain that is at least 90% identical to SEQ ID NO: 336, where the light chain variable domain includes a histidine at one or more positions in SEQ ID NO: 336 selected from the group consisting of: 27, 29, 32, 34, 52, 53, 89, 90, 91, 92, 93, 94, and 96; (e) a heavy chain variable domain that is at least 90% identical to SEQ ID NO: 401, where the heavy chain variable domain includes a histidine at one or more positions in SEQ ID NO: 401 selected from the group consisting of: 27, 101, 103, 104, 108, and 109; and/or a light chain variable domain that is at least 90% identical to SEQ ID NO: 402, where the light chain variable domain includes a histidine at one or more positions in SEQ ID NO: 402 selected from the group consisting of: 30 and 31; (f) a heavy chain variable domain that is at least 90% identical to SEQ ID NO: 470, where the heavy chain variable domain includes a histidine at one or more positions in SEQ ID NO: 470 selected from the group consisting of 24, 27, 29, 32, 34, 35, 50, 51, 53, 54, 56, 58, 59, 60, 63, 98, 101, 103, 105, 106, and 107; and/or a light chain variable domain that is at least 90% identical to SEQ ID NO: 471, where the light chain variable domain includes a histidine at one or more positions in SEQ ID NO: 471 selected from the group consisting of: 29, 32, 33, 34, 50, 51, 53, 55, 89, 92, 94, 96 and 97; and (g) a heavy chain variable domain that is at least 90% identical to SEQ ID NO: 537, where the heavy chain variable domain includes a histidine at one or more positions in SEQ ID NO: 537 selected from the group consisting of 26, 29, 31, 32, 53, 54, 57, 58, 59, 65, 67, 68, 103, 104, and 106; and/or a light chain variable domain that is at least 90% identical to SEQ ID NO: 538, where the light chain variable domain includes a histidine at one or more positions in SEQ ID NO: 538 selected from the group consisting of: 28, 34, 53, 93, 94, and 98.

In some embodiments of any of the ABPCs described herein, the first antigen-binding domain includes one of (a) through (g): (a) a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 65, SEQ ID NO: 66, SEQ ID NO: 69, SEQ ID NO: 72, SEQ ID NO: 73, SEQ ID NO: 76, SEQ ID NO: 77, SEQ ID NO: 78, SEQ ID NO: 79, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, or SEQ ID NO: 118, and/or a heavy chain variable domain of SEQ ID NO: 1, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, SEQ ID NO: 52, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98, where the first antigen-binding domain does not comprise (i) a light chain variable domain of SEQ ID NO: 2 and a heavy chain variable domain of SEQ ID NO: 1; (ii) a light chain variable domain of SEQ ID NO: 2 and heavy chain variable domain that is not one of SEQ ID NOs: 17, 19, 21, 22, 24-26, 29-31, 34, 43, 44, 47, 49, 51, 52, and 81-98; or (iii) a heavy chain variable domain of SEQ ID NO: 1 and a light chain variable domain that is not one of SEQ ID NOs: 55, 56, 59, 62-66, 69, 72, 73, 76-79, and 99-118; (b) a light chain variable domain of SEQ ID NO: 120, SEQ ID NO: 163, SEQ ID NO: 166, SEQ ID NO: 168, SEQ ID NO: 178, SEQ ID NO: 179, SEQ ID NO: 180, SEQ ID NO: 181, SEQ ID NO: 183, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, or SEQ ID NO: 235, and/or a heavy chain variable domain of SEQ ID NO: 119, SEQ ID NO: 127, SEQ ID NO: 130, SEQ ID NO: 131, SEQ ID NO: 133, SEQ ID NO: 134, SEQ ID NO: 138, SEQ ID NO: 139, SEQ ID NO: 140, SEQ ID NO: 141, SEQ ID NO: 143, SEQ ID NO: 145, SEQ ID NO: 146, SEQ ID NO: 148, SEQ ID NO: 149, SEQ ID NO: 150, SEQ ID NO: 151, SEQ ID NO: 153, SEQ ID NO: 155, SEQ ID NO: 156; SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 216, SEQ ID NO: 217, SEQ ID NO: 218, SEQ ID NO: 220, SEQ ID NO: 221, or SEQ ID NO: 223, where the first antigen-binding domain does not comprise (i) a light chain variable domain of SEQ ID NO: 120 and a heavy chain variable domain of SEQ ID NO: 119; (ii) a light chain variable domain of SEQ ID NO: 120 and heavy chain variable domain that is not one of SEQ ID NOs: 127, 130, 131, 133, 134, 138-141, 143, 145, 146, 148-151, 153, 155, 156, 185-214, 216-218, 220, 221, and 223; or (iii) a heavy chain variable domain of SEQ ID NO: 119 and a light chain variable domain that is not one of SEQ ID NOs: 163, 166, 168, 178-181, 183, and 226-235; (c) a light chain variable domain of SEQ ID NO: 237, SEQ ID NO: 283, SEQ ID NO: 287, SEQ ID NO: 290, SEQ ID NO: 291, SEQ ID NO: 292, SEQ ID NO: 293, SEQ ID NO: 301, SEQ ID NO: 302, SEQ ID NO: 304, SEQ ID NO: 306, or SEQ ID NO: 334; and/or a heavy chain variable domain of SEQ ID NO: 236, SEQ ID NO: 239, SEQ ID NO: 241, SEQ ID NO: 243, SEQ ID NO: 245, SEQ ID NO: 246, SEQ ID NO: 247, SEQ ID NO: 248, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 268, SEQ ID NO: 270, SEQ ID NO: 274, SEQ ID NO: 276, SEQ ID NO: 278, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 330, SEQ ID NO: 331, or SEQ ID NO: 332, where the first antigen-binding domain does not comprise (i) a light chain variable domain of SEQ ID NO: 237 and a heavy chain variable domain of SEQ ID NO: 236; (ii) a light chain variable domain of SEQ ID NO: 237 and heavy chain variable domain that is not one of SEQ ID NOs: 239, 241, 243, 245-250, 252-254, 256, 258, 266, 268, 270, 274, 276, 278, 308-328, and 330-332; or (iii) a heavy chain variable domain of SEQ ID NO: 236 and a light chain variable domain that is not one of SEQ ID NOs: 283, 287, 290-293, 301, 302, 304, 306, and 334;

(d) a light chain variable domain of SEQ ID NO: 336, SEQ ID NO: 377, SEQ ID NO: 379, SEQ ID NO: 382, SEQ ID NO: 384, SEQ ID NO: 387, SEQ ID NO: 388, SEQ ID NO: 392, SEQ ID NO: 393, SEQ ID NO: 394, SEQ ID NO: 395, SEQ ID NO: 396, SEQ ID NO: 397, or SEQ ID NO: 399, and/or a heavy chain variable domain of SEQ ID NO: 335, SEQ ID NO: 341, SEQ ID NO: 346, SEQ ID NO: 347, SEQ ID NO: 348, SEQ ID NO: 349, SEQ ID NO: 350, SEQ ID NO: 368, SEQ ID NO: 371, SEQ ID NO: 372, SEQ ID NO: 373, SEQ ID NO: 374, or SEQ ID NO: 375, where the first antigen-binding domain does not comprise (i) a light chain variable domain of SEQ ID NO: 336 and a heavy chain variable domain of SEQ ID NO: 335; (ii) a light chain variable domain of SEQ ID NO: 336 and heavy chain variable domain that is not one of SEQ ID NOs: 341, 346-350, 368, and 371-375; or (iii) a heavy chain variable domain of SEQ ID NO: 335 and a light chain variable domain that is not one of SEQ ID NOs: 377, 379, 382, 384, 387, 388, 392-397, and 399; (e) a light chain variable domain of SEQ ID NO: 402, SEQ ID NO: 450, or SEQ ID NO: 451; and/or a heavy chain variable domain of SEQ ID NO: 401, SEQ ID NO: 404, SEQ ID NO: 434, SEQ ID NO: 436, SEQ ID NO: 437, SEQ ID NO: 441, or SEQ ID NO: 442, where the first antigen-binding domain does not comprise (i) a light chain variable domain of SEQ ID NO: 402 and a heavy chain variable domain of SEQ ID NO: 401; (ii) a light chain variable domain of SEQ ID NO: 402 and heavy chain variable domain that is not one of SEQ ID NOs: 404, 434, 436, 437, 441, and 442; or (iii) a heavy chain variable domain of SEQ ID NO: 401 and a light chain variable domain that is not one of SEQ ID NO: 450 or 451;
(f) a light chain variable domain of SEQ ID NO: 471, SEQ ID NO: 515, SEQ ID NO: 518, SEQ ID NO: 519, SEQ ID NO: 520, SEQ ID NO: 521, SEQ ID NO: 522, SEQ ID NO: 524, SEQ ID NO: 526, SEQ ID NO: 528, SEQ ID NO: 531, SEQ ID NO: 533, SEQ ID NO: 535, or SEQ ID NO: 536, and/or a heavy chain variable domain of SEQ ID NO: 470, SEQ ID NO: 473, SEQ ID NO: 476, SEQ ID NO: 478, SEQ ID NO: 481, SEQ ID NO: 483, SEQ ID NO: 484, SEQ ID NO: 485, SEQ ID NO: 486, SEQ ID NO: 488, SEQ ID NO: 489, SEQ ID NO: 491, SEQ ID NO: 493, SEQ ID NO: 494, SEQ ID NO: 495, SEQ ID NO: 498, SEQ ID NO: 503, SEQ ID NO: 506, SEQ ID NO: 508, SEQ ID NO: 510, SEQ ID NO: 511, or SEQ ID NO: 512, where the first antigen-binding domain does not comprise (i) a light chain variable domain of SEQ ID NO: 471 and a heavy chain variable domain of SEQ ID NO: 470; (ii) a light chain variable domain of SEQ ID NO: 471 and heavy chain variable domain that is not one of SEQ ID NOs: 473, 476, 478, 481, 483-486, 488, 489, 491, 493-495, 498, 503, 506, 508, and 510-512; or (iii) a heavy chain variable domain of SEQ ID NO: 470 and a light chain variable domain that is not one of SEQ ID NOs: 515, 518-522, 524, 526, 528, 531, 533, 535, and 536; and (g) a light chain variable domain of SEQ ID NO: 538, SEQ ID NO: 581, SEQ ID NO: 587, SEQ ID NO: 591, SEQ ID NO: 599, SEQ ID NO: 600, or SEQ ID NO: 604, and/or a heavy chain variable domain of SEQ ID NO: 537, SEQ ID NO: 539, SEQ ID NO: 542, SEQ ID NO: 544, SEQ ID NO: 545, SEQ ID NO: 552, SEQ ID NO: 553, SEQ ID NO: 556, SEQ ID NO: 557, SEQ ID NO: 558, SEQ ID NO: 564, SEQ ID NO: 566, SEQ ID NO: 567, SEQ ID NO: 572, SEQ ID NO: 573, or SEQ ID NO: 575, where the first antigen-binding domain does not comprise (i) a light chain variable domain of SEQ ID NO: 538 and a heavy chain variable domain of SEQ ID NO: 537; (ii) a light chain variable domain of SEQ ID NO: 538 and heavy chain variable domain that is not one of SEQ ID NOs: 539, 542, 544, 545, 552, 553, 556-558, 564, 566, 567, 572, 573, and 575; or (iii) a heavy chain variable domain of SEQ ID NO: 537 and a light chain variable domain that is not one of SEQ ID NOs: 581, 587, 591, 599, 600, and 604.

In some embodiments of any of the ABPCs described herein, a composition including the ABPC provides for an increase in toxin liberation in the target mammalian cell as compared to a composition including the same amount of a control ABPC and/or an increase in target mammalian cell killing as compared to a composition including the same amount of a control ABPC.

In some embodiments of any of the ABPCs described herein, a composition including the ABPC provides for an increase in endolysosomal delivery in the target mammalian cell as compared to a composition including the same amount of a control ABPC.

In some embodiments of any of the ABPCs described herein, a composition including the ABPC results in a less of a reduction in the level of DLL3 presented on the surface of the target mammalian cell as compared to a composition including the same amount of a control ABPC; or does not result in a detectable reduction in the level of DLL3 presented on the surface of the target mammalian cell.

In some embodiments of any of the ABPCs described herein, the target mammalian cell is a cancer cell. In some embodiments of any of the ABPCs described herein, the ABPC is cytotoxic or cytostatic to the target mammalian cell.

In some embodiments of any of the ABPCs described herein, ABPC is cross-reactive with a non-human primate DLL3 and human DLL3 or cross-reactive with a non-human primate DLL3, a human DLL3, and one or both of rat DLL3 and a mouse DLL3.

In some embodiments of any of the ABPCs described herein, the ABPC includes a single polypeptide. In some embodiments of any of the ABPCs described herein, the antigen-binding domain is selected from the group consisting of: a VH domain, a VHH domain, a VNAR domain, and a scFv. In some embodiments of any of the ABPCs described herein, the ABPC includes two or more polypeptides. In some embodiments of any of the ABPCs described herein, the ABPC is an antibody.

In some embodiments of any of the ABPCs described herein, the half-life of the ABPC in vivo is decreased as compared to the half-life of a control ABPC in vivo.

In some embodiments of any of the ABPCs described herein, the ABPC includes a second antigen-binding domain.

Also provided herein are kits including at least one dose of the pharmaceutical composition of any of the pharmaceutical compositions described herein or any of the ABPCs described herein.

Also provided here are methods of treating a cancer characterized by having a population of cancer cells that have DLL3 or an epitope of DLL3 presented on their surface, the method including, administering a therapeutically effective amount of any one of the pharmaceutical compositions described herein or any one of the ABPCs described herein to a subject identified as having a cancer characterized by having the population of cancer cells.

Also provided herein, are methods of reducing the volume of a tumor in a subject, where the tumor is characterized by having a population of cancer cells that have DLL3 or an epitope of DLL3 presented on their surface, the method including, administering a therapeutically effective amount of any of the pharmaceutical compositions described herein or any one of the ABPCs described herein to a subject identified as having a cancer characterized by having the population of cancer cells.

Also provided herein are methods of inducing cell death in a cancer cell in a subject, where the cancer cell has DLL3 or an epitope of DLL3 presented on its surface, where the method includes administering a therapeutically effective amount of any of the pharmaceutical compositions described herein or any one of the ABPCs described herein to a subject identified as having a cancer characterized by having a population of the cancer cells.

Also provided herein are methods of decreasing the risk of developing a metastasis or decreasing the risk of developing an additional metastasis in a subject having a cancer, wherein the cancer is characterized by having a population of cancer cells that have DLL3 or an epitope of DLL3 presented on their surface the method including, administering a therapeutically effective amount of any one of the pharmaceutical compositions described herein or any one of the ABPCs described herein to a subject identified as having a cancer characterized by having the population of cancer cells.

As used herein, the term “antigen-binding protein construct” is (i) a single polypeptide that includes at least one antigen-binding domain or (ii) a complex of two or more polypeptides (e.g., the same or different polypeptides) that together form at least one antigen-binding domain. Non-limiting examples and aspects of antigen-binding protein constructs are described herein. Additional examples and aspects of antigen-binding protein constructs are known in the art.

A “multi-specific antigen-binding protein construct” is an antigen-binding protein construct that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, the antigen is present on the surface of the cell. In some aspects, a multi-specific antigen-binding protein construct binds two different epitopes (i.e., a “bispecific antigen-binding protein construct”). In some aspects, a multi-specific antigen-binding protein construct binds three different epitopes (i.e., a “trispecific antigen-binding protein construct”). In some aspects, a multi-specific antigen-binding protein construct binds four different epitopes (i.e., a “quadspecific antigen-binding protein construct”). In some aspects, a multi-specific antigen-binding protein construct binds five different epitopes (i.e., a “quintspecific antigen-binding protein construct”). Each binding specificity may be present in any suitable valency. Non-limiting examples of multi-specific antigen-binding protein constructs are described herein.

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art. In some examples, an antigen-binding domain can bind to a single antigen.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins, e.g., human IgG (e.g., human IgG1, human IgG2, human IgG3, human IgG4)), antibody fragments, and multi-specific antibodies. One example of an antigen-binding domain is an antigen-binding domain formed by a VH-VL dimer. Additional examples of an antibody are described herein. Additional examples of an antibody are known in the art.

The phrase “endosomal/lysosomal pathway” refers to a network of endosomes (early endosomes, multi-vesicular bodies, late endosomes, and lysosomes) in the cytoplasm of a mammalian cell, wherein molecules internalized through cell-mediated internalization processes, e.g., pinocytosis, micropinocytosis, receptor-mediated endocytosis, and/or phagocytosis, are sorted.

Once the endosomes in the endosomal/lysosomal pathway are purified or isolated, assays for a target protein (e.g., an antigen-binding protein construct described herein) can be performed using methods known in the art (ELISA, Western blot, immunofluorescence, and immunoprecipitation followed by an assay for protein concentration), and can be used to determine the concentration or relative level of the target protein in the endosomes. Alternatively, endosomes in the endosomal/lysosomal pathway can be imaged using immunofluorescence microscopy using an detectably-labelled antibody (e.g., a fluorophore-labelled, a dye-labelled, or a GFP-labelled antibody, e.g., CellLight™ Early Endosome-GFP) that specifically binds to a characteristic protein present in the endosomes (e.g., EEA1 for early endosomes) and a fluorophore-labelled antibody that specifically binds to the protein of interest (e.g., an antigen-binding protein construct), and the level of the target protein in the endosomes can be determined by quantitation of the overlap in the fluorescence emissions of the two different antibodies.

The phrase “endolysosomal delivery” refers to rate of accumulation over time or the total accumulation at a specific timepoint of an antigen-binding protein construct (e.g., any of the antigen-binding protein constructs described herein) in the endosomal/lysosomal pathway in a mammalian cell (e.g., any of the exemplary target mammalian cells described herein).

An exemplary method to calculate the increase in endolysosomal delivery of a pH engineered ABPC variant as compared to its corresponding starting ABPC from cellular fluorescence data is to measure the ratio of the variant's mean fluorescence intensity minus the mean fluorescence intensity of a non-binding IgG control, then all divided by the variant's corresponding starting ABPC's mean fluorescence intensity minus the mean fluorescence intensity of the IgG control.

An exemplary assay for measuring endolysosomal delivery of any of the ABPCs described herein include those which involve labeling of an ABPC with a fluorescent dye, followed by incubation of the labeled ABPC with cells and measurement of cellular fluorescence as an indicator of endolysosomal delivery of the ABPC (e.g., as described generally in Wustner, Traffic 7(6):699-715, 2006). Alternatively, pH-sensitive dyes which preferentially fluoresce at acidic pH but not neutral pH can be used to label any of the ABPCs described herein, which can then be incubated with cells and the cellular fluorescence measured as an indicator of delivery of the ABPC into acidic endolysosomal compartments.

The term “population” when used before a noun means two or more of the specific noun. For example, the phrase “a population of cancer cells” means “two or more cancer cells.” Non-limiting examples of cancer cells are described herein.

The phrase “cytostatic to a cell” refers to a direct or indirect decrease in the proliferation (cell division) of the cell (e.g., a cancer cell) in vivo or in vitro. When an agent is cytostatic to a cell, the agent can, e.g., directly or indirectly result in cell cycle arrest of the cell (e.g., a cancer cell). In some examples, an agent that is cytostatic to a cell can reduce the number of cells in a population of the cells that are in S phase (as compared to the number of cells in a population of the cells that are in S phase prior to contact with the agent). In some examples, an agent that is cytostatic to a cell can reduce the percentage of the cells in S phase by at least 20%, at least 40%, at least 60%, or at least 80% (e.g., as compared to the percentage of cells in a population of the cells that are in S phase prior to contact with the agent).

The phrase “cytotoxic to a cell” refers to the inducement, directly or indirectly, in the death (e.g., necrosis or apoptosis) of the cell (e.g., a mammalian cell, e.g., a cancer cell).

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

The term “epitope” means a portion of an antigen that is specifically bound by an antigen-binding domain through a set of physical interactions between: (i) all monomers (e.g. individual amino acid residues, sugar side chains, and post-translationally modified amino acid residues) on the portion of the antigen-binding domain that specifically binds the antigen and (ii) all monomers (e.g. individual amino acid residues, sugar side chains, post-translationally modified amino acid residues) on the portion of the antigen that is specifically bound by the antigen-binding domain. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and other amino acid residues, which are not directly involved in the binding.

Methods for identifying an epitope to which an antigen-binding domain specifically binds are known in the art, e.g., structure-based analysis (e.g. X-ray crystallography, NMR, and/or electron microscopy) (e.g. on the antigen and/or the antigen-antigen binding domain complex) and/or mutagenesis-based analysis (e.g. alanine scanning mutagenesis, glycine scanning mutagenesis, and homology scanning mutagenesis) wherein mutants are measured in a binding assay with a binding partner, many of which are known in the art.

The term “paratope” means a portion of an antigen-binding domain that specifically binds to an antigen through a set of physical interactions between: (i) all monomers (e.g. individual amino acid residues, sugar side chains, posttranslationally modified amino acid residues) on the portion of the antigen-binding domain that specifically binds the antigen and (ii) all monomers (e.g. individual amino acid residues, sugar side chains, posttranslationally modified amino acid residues) on the portion of the antigen that is specifically bound by the antigen-binding domain. Paratopes can, e.g. consist of surface-accessible amino acid residues and may have specific three-dimensional structural characteristics, as well as specific charge characteristics. In some embodiments, a paratope refers to a portion of a full-length antigen-binding domain or a portion thereof that is defined by a three-dimensional structure (e.g., protein folding). In some embodiments, a paratope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the paratope may not be critical to three-dimensional structure of the paratope. A paratope may comprise amino acid residues that are directly involved in the binding, and other amino acid residues, which are not directly involved in the binding.

Methods for identifying a paratope to which an antigen-binding domain specifically binds are known in the art, e.g., structure-based analysis (e.g., X-ray crystallography, NMR, and/or electron microscopy) (e.g. on the antigen-binding domain, and/or the antigen binding domain-antigen complex), and/or mutagenesis-based analysis (e.g., alanine scanning mutagenesis, glycine scanning mutagenesis, and homology scanning mutagenesis) wherein mutants are measured in a binding assay with a binding partner, many of which are known in the art.

The phrase “present on the surface of a mammalian cell” means (1) an antigen that physically attached to or at least partially embedded in the plasma membrane of a mammalian cell (e.g., a transmembrane protein, a peripheral membrane protein, a lipid-anchored protein (e.g., a GPI-anchor), an N-myristolyated protein, or a S-palmitoylated protein) or (2) an antigen that is stably bound to its cognate receptor, where the cognate receptor is physically attached to the plasma membrane of a mammalian cell (e.g., a ligand bound to its cognate receptor, where the cognate receptor is physically attached to the plasma membrane). Non-limiting methods for determining the presence of antigen on the surface of a mammalian cell include fluorescence-activated cell sorting (FACS), immunohistochemistry, cell-fractionation assays and Western blotting.

The phrase “control ABPC” or “control antigen-binding protein construct” means (i) an ABPC that is capable of specifically binding to DLL3 or an epitope of DLL3 presented on the surface of a mammalian cell (e.g., a target mammalian cell), where one or both of the following is true: (a) the dissociation rate of the first antigen-binding domain at a pH of about 4.0 to about 6.5 (e.g., any of the subranges of this range described herein) is no more than 3-fold (e.g., no more than 2.8-fold, no more than 2.6-fold, no more than 2.5-fold, no more than 2.4-fold, no more than 2.2-fold, no more than 2.0-fold, no more than 1.8-fold, no more than 1.6-fold, no more than 1.5-fold, no more than 1.4-fold, no more than 1.2-fold, no more than 1.0-fold, no more than 0.8-fold, no more than 0.6-fold, no more than 0.5-fold, no more than 0.4-fold, no more than 0.3-fold no more than 0.2-fold, or no more than 0.1-fold) faster than the dissociation rate at a pH of about 7.0 to about 8.0 (e.g., any of the subranges of this range described herein); or (b) the dissociation constant (K_{D}) of the first antigen-binding domain at a pH of about 4.0 to about 6.5 (e.g., any of the subranges of this range described herein) is no more than 3-fold (e.g., no more than 2.8-fold, no more than 2.6-fold, no more than 2.5-fold, no more than 2.4-fold, no more than 2.2-fold, no more than 2.0-fold, no more than 1.8-fold, no more than 1.6-fold, no more than 1.5-fold, no more than 1.4-fold, no more than 1.2-fold, no more than 1.0-fold, no more than 0.8-fold, no more than 0.6-fold, no more than 0.5-fold, no more than 0.4-fold, no more than 0.3-fold no more than 0.2-fold, or no more than 0.1-fold) greater than the K_{D} at a pH of about 7.0 to about 8.0 (e.g., any of the subranges of this range described herein); and/or (ii) rovalpituzumab, SC16.4, SC16.13, SC16.15, SC16.25, SC16.34, or SC16.67.

The term “extracellular space” means the liquid exterior to the plasma membrane of a mammalian cell. When a mammalian cell is in vitro, the extracellular space can be a liquid culture medium. When a mammalian cell is in vivo, the extracellular space can be, e.g., plasma, serum, blood, interstitial fluid, or lymph.

The term “endolysosomal space” means the fluid encapsulated by the vesicles and organelles that make-up the endosomal/lysosomal pathway in a mammalian cell.

The phrase “a reduced level” or “a decreased level” can be a reduction or decrease of at least a 1% (e.g., at least 2%, at least 4%, at least 6%, at least 8%, at least 10%, at least 12%, at least 14%, at least 16%, at least 18%, at least 20%, at least 22%, at least 24%, at least 26%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) reduction as compared to a reference level or value.

The term “cell killing potency” refers to the ability of an agent (e.g., any of the ABPCs described herein) to induce, directly or indirectly, the apoptosis and/or necrosis of a mammalian cell (e.g., a cancer cell), measured as a rate over time or at a relevant timepoint. Methods for determining the cell killing potency of a cell are known in the art (e.g., trypan blue staining, microscopy, fluorescence-assisted cell sorting, and assays to detect markers of apoptosis (e.g., Annexin V)). In non-limiting examples, cell killing potency can be measured, e.g., by cell killing at a single concentration of an agent, by the IC50 of the agent (i.e. the concentration of the agent whereby half the maximal cell killing potency is achieved), or by the ratio of an agent's dissociation constant KD on mammalian cells divided by its IC50. In some non-limiting examples, the IC50s and/or the KD ratios described herein are compared to those of a control ABPC (as defined herein), and, optionally, demonstrate that the ABPCs described herein have a higher cell killing potency as compared to the control ABPC.

The term “toxin liberation” refers to the ability of a mammalian cell (e.g., a non-cancerous mammalian cell or a cancer cell) to internalize (e.g., via pinocytosis and/or receptor-mediated endocytosis) any of the ABPCs described herein (e.g., any of ABPCs or control ABPCs described herein) that are conjugated to a toxin, and subsequently release the toxin conjugated to the ABPC, measured as a rate over time or at a specific timepoint. Toxin liberation can be assessed using a variety of different exemplary assays, e.g., ELISA, immunofluorescence, cell killing assays, cell cycle arrest assays, DNA damage assays, mass spectrometry, HPLC, and/or an isotope-labeled toxin.

The phrase “target cell” or “target mammalian cell” or “mammalian target cell” means a mammalian cell that has at least one DLL3 present on its surface. In some examples, a mammalian target cell can be a cancer cell. In some embodiments of a target mammalian cell can have a total of about 1 to about 10,000,000, about 1 to about 9,000,000, about 1 to about 8,000,000, about 1 to about 7,000,000, about 1 to about 6,000,000, about 1 to about 5,000,000, about 1 to about 4,000,000, about 1 to about 3,000,000, about 1 to about 2,000,000, about 1 to about 1,000,000, about 1 to about 800,000, about 1 to about 600,000, about 1 to about 400,000, about 1 to about 200,000, about 1 to about 100,000, about 1 to about 80,000, about 1 to about 80,000, about 1 to about 75,000, about 1 to about 70,000, about 1 to about 65,000, about 1 to about 60,000, about 1 to about 55,000, about 1 to about 50,000, about 1 to about 45,000, about 1 to about 40,000, about 1 to about 35,000, about 1 to about 30,000, about 1 to about 25,000, about 1 to about 20,000, about 1 to about 15,000, about 1 to about 10,000, about 1 to about 7,500, about 1 to about 5,000, about 1 to about 4,000, about 1 to about 3,000, about 1 to about 2,000, about 1 to about 1,000, about 1 to about 500, about 1 to about 100, about 1 to about 50, about 1 to about 10, about 10 to about 10,000,000, about 10 to about 9,000,000, about 10 to about 8,000,000, about 10 to about 7,000,000, about 10 to about 6,000,000, about 10 to about 5,000,000, about 10 to about 4,000,000, about 10 to about 3,000,000, about 10 to about 2,000,000, about 10 to about 1,000,000, about 10 to about 800,000, about 10 to about 600,000, about 10 to about 400,000, about 10 to about 200,000, about 10 to about 100,000, about 10 to about 80,000, about 10 to about 80,000, about 10 to about 75,000, about 10 to about 70,000, about 10 to about 65,000, about 10 to about 60,000, about 10 to about 55,000, about 10 to about 50,000, about 10 to about 45,000, about 10 to about 40,000, about 10 to about 35,000, about 10 to about 30,000, about 10 to about 25,000, about 10 to about 20,000, about 10 to about 15,000, about 10 to about 10,000, about 10 to about 7,500, about 10 to about 5,000, about 10 to about 4,000, about 10 to about 3,000, about 10 to about 2,000, about 10 to about 1,000, about 10 to about 500, about 10 to about 100, about 10 to about 50, about 50 to about 10,000,000, about 50 to about 9,000,000, about 50 to about 8,000,000, about 50 to about 7,000,000, about 50 to about 6,000,000, about 50 to about 5,000,000, about 50 to about 4,000,000, about 50 to about 3,000,000, about 50 to about 2,000,000, about 50 to about 1,000,000, about 50 to about 800,000, about 50 to about 600,000, about 50 to about 400,000, about 50 to about 200,000, about 50 to about 100,000, about 50 to about 80,000, about 50 to about 80,000, about 50 to about 75,000, about 50 to about 70,000, about 50 to about 65,000, about 50 to about 60,000, about 50 to about 55,000, about 50 to about 50,000, about 50 to about 45,000, about 50 to about 40,000, about 50 to about 35,000, about 50 to about 30,000, about 50 to about 25,000, about 50 to about 20,000, about 50 to about 15,000, about 50 to about 10,000, about 50 to about 7,500, about 50 to about 5,000, about 50 to about 4,000, about 50 to about 3,000, about 50 to about 2,000, about 50 to about 1,000, about 50 to about 500, about 50 to about 100, about 100 to about 10,000,000, about 100 to about 9,000,000, about 100 to about 8,000,000, about 100 to about 7,000,000, about 100 to about 6,000,000, about 100 to about 5,000,000, about 100 to about 4,000,000, about 100 to about 3,000,000, about 100 to about 2,000,000, about 100 to about 1,000,000, about 100 to about 800,000, about 100 to about 600,000, about 100 to about 400,000, about 100 to about 200,000, about 100 to about 100,000, about 100 to about 80,000, about 100 to about 75,000, about 100 to about 70,000, about 100 to about 65,000, about 100 to about 60,000, about 100 to about 55,000, about 100 to about 50,000, about 100 to about 45,000, about 100 to about 40,000, about 100 to about 35,000, about 100 to about 30,000, about 100 to about 25,000, about 100 to about 20,000, about 100 to about 15,000, about 100 to about 10,000, about 100 to about 7,500, about 100 to about 5,000, about 100 to about 4,000, about 100 to about 3,000, about 100 to about 2,000, about 100 to about 1,000, about 100 to about 500, about 500 to about 10,000,000, about 500 to about 9,000,000, about 500 to about 8,000,000, about 500 to about 7,000,000, about 500 to about 6,000,000, about 500 to about 5,000,000, about 500 to about 4,000,000, about 500 to about 3,000,000, about 500 to about 2,000,000, about 500 to about 1,000,000, about 500 to about 800,000, about 500 to about 600,000, about 500 to about 400,000, about 500 to about 200,000, about 500 to about 100,000, about 500 to about 80,000, about 500 to about 75,000, about 500 to about 70,000, about 500 to about 65,000, about 500 to about 60,000, about 500 to about 55,000, about 500 to about 50,000, about 500 to about 45,000, about 500 to about 40,000, about 500 to about 35,000, about 500 to about 30,000, about 500 to about 25,000, about 500 to about 20,000, about 500 to about 15,000, about 500 to about 10,000, about 500 to about 7,500, about 500 to about 5,000, about 500 to about 4,000, about 500 to about 3,000, about 500 to about 2,000, about 500 to about 1,000, about 1,000 to about 10,000,000, about 1,000 to about 9,000,000, about 1,000 to about 8,000,000, about 1,000 to about 7,000,000, about 1,000 to about 6,000,000, about 1,000 to about 5,000,000, about 1,000 to about 4,000,000, about 1,000 to about 3,000,000, about 1,000 to about 2,000,000, about 1,000 to about 1,000,000, about 1,000 to about 800,000, about 1,000 to about 600,000, about 1,000 to about 400,000, about 1,000 to about 200,000, about 1,000 to about 100,000, about 1,000 to about 80,000, about 1,000 to about 75,000, about 1,000 to about 70,000, about 1,000 to about 65,000, about 1,000 to about 60,000, about 1,000 to about 55,000, about 1,000 to about 50,000, about 1,000 to about 45,000, about 1,000 to about 40,000, about 1,000 to about 35,000, about 1,000 to about 30,000, about 1,000 to about 25,000, about 1,000 to about 20,000, about 1,000 to about 15,000, about 1,000 to about 10,000, about 1,000 to about 7,500, about 1,000 to about 5,000, about 1,000 to about 4,000, about 1,000 to about 3,000, about 1,000 to about 2,000, about 2,000 to about 10,000,000, about 2,000 to about 9,000,000, about 2,000 to about 8,000,000, about 2,000 to about 7,000,000, about 2,000 to about 6,000,000, about 2,000 to about 5,000,000, about 2,000 to about 4,000,000, about 2,000 to about 3,000,000, about 2,000 to about 2,000,000, about 2,000 to about 1,000,000, about 2,000 to about 800,000, about 2,000 to about 600,000, about 2,000 to about 400,000, about 2,000 to about 200,000, about 2,000 to about 100,000, about 2,000 to about 80,000, about 2,000 to about 75,000, about 2,000 to about 70,000, about 2,000 to about 65,000, about 2,000 to about 60,000, about 2,000 to about 55,000, about 2,000 to about 50,000, about 2,000 to about 45,000, about 2,000 to about 40,000, about 2,000 to about 35,000, about 2,000 to about 30,000, about 2,000 to about 25,000, about 2,000 to about 20,000, about 2,000 to about 15,000, about 2,000 to about 10,000, about 2,000 to about 7,500, about 2,000 to about 5,000, about 2,000 to about 4,000, about 2,000 to about 3,000, about 3,000 to about 10,000,000, about 3,000 to about 9,000,000, about 3,000 to about 8,000,000, about 3,000 to about 7,000,000, about 3,000 to about 6,000,000, about 3,000 to about 5,000,000, about 3,000 to about 4,000,000, about 3,000 to about 3,000,000, about 3,000 to about 2,000,000, about 3,000 to about 1,000,000, about 3,000 to about 800,000, about 3,000 to about 600,000, about 3,000 to about 400,000, about 3,000 to about 200,000, about 3,000 to about 100,000, about 3,000 to about 80,000, about 3,000 to about 75,000, about 3,000 to about 70,000, about 3,000 to about 65,000, about 3,000 to about 60,000, about 3,000 to about 55,000, about 3,000 to about 50,000, about 3,000 to about 45,000, about 3,000 to about 40,000, about 3,000 to about 35,000, about 3,000 to about 30,000, about 3,000 to about 25,000, about 3,000 to about 20,000, about 3,000 to about 15,000, about 3,000 to about 10,000, about 3,000 to about 7,500, about 3,000 to about 5,000, about 3,000 to about 4,000, about 4,000 to about 10,000,000, about 4,000 to about 9,000,000, about 4,000 to about 8,000,000, about 4,000 to about 7,000,000, about 4,000 to about 6,000,000, about 4,000 to about 5,000,000, about 4,000 to about 4,000,000, about 4,000 to about 3,000,000, about 4,000 to about 2,000,000, about 4,000 to about 1,000,000, about 4,000 to about 800,000, about 4,000 to about 600,000, about 4,000 to about 400,000, about 4,000 to about 200,000, about 4,000 to about 100,000, about 4,000 to about 80,000, about 4,000 to about 75,000, about 4,000 to about 70,000, about 4,000 to about 65,000, about 4,000 to about 60,000, about 4,000 to about 55,000, about 4,000 to about 50,000, about 4,000 to about 45,000, about 4,000 to about 40,000, about 4,000 to about 35,000, about 4,000 to about 30,000, about 4,000 to about 25,000, about 4,000 to about 20,000, about 4,000 to about 15,000, about 4,000 to about 10,000, about 4,000 to about 7,500, about 4,000 to about 5,000, about 5,000 to about 10,000,000, about 5,000 to about 9,000,000, about 5,000 to about 8,000,000, about 5,000 to about 7,000,000, about 5,000 to about 6,000,000, about 5,000 to about 5,000,000, about 5,000 to about 4,000,000, about 5,000 to about 3,000,000, about 5,000 to about 2,000,000, about 5,000 to about 1,000,000, about 5,000 to about 800,000, about 5,000 to about 600,000, about 5,000 to about 400,000, about 5,000 to about 200,000, about 5,000 to about 100,000, about 5,000 to about 80,000, about 5,000 to about 75,000, about 5,000 to about 70,000, about 5,000 to about 65,000, about 5,000 to about 60,000, about 5,000 to about 55,000, about 5,000 to about 50,000, about 5,000 to about 45,000, about 5,000 to about 40,000, about 5,000 to about 35,000, about 5,000 to about 30,000, about 5,000 to about 25,000, about 5,000 to about 20,000, about 5,000 to about 15,000, about 5,000 to about 10,000, about 5,000 to about 7,500, about 7,500 to about 10,000,000, about 7,500 to about 9,000,000, about 7,500 to about 8,000,000, about 7,500 to about 7,000,000, about 7,500 to about 6,000,000, about 7,500 to about 5,000,000, about 7,500 to about 4,000,000, about 7,500 to about 3,000,000, about 7,500 to about 2,000,000, about 7,500 to about 1,000,000, about 7,500 to about 800,000, about 7,500 to about 600,000, about 7,500 to about 400,000, about 7,500 to about 200,000, about 7,500 to about 100,000, about 7,500 to about 80,000, about 7,500 to about 75,000, about 7,500 to about 70,000, about 7,500 to about 65,000, about 7,500 to about 60,000, about 7,500 to about 55,000, about 7,500 to about 50,000, about 7,500 to about 45,000, about 7,500 to about 40,000, about 7,500 to about 35,000, about 7,500 to about 30,000, about 7,500 to about 25,000, about 7,500 to about 20,000, about 7,500 to about 15,000, about 7,500 to about 10,000, about 10,000 to about 10,000,000, about 10,000 to about 9,000,000, about 10,000 to about 8,000,000, about 10,000 to about 7,000,000, about 10,000 to about 6,000,000, about 10,000 to about 5,000,000, about 10,000 to about 4,000,000, about 10,000 to about 3,000,000, about 10,000 to about 2,000,000, about 10,000 to about 1,000,000, about 10,000 to about 800,000, about 10,000 to about 600,000, about 10,000 to about 400,000, about 10,000 to about 200,000, about 10,000 to about 100,000, about 10,000 to about 80,000, about 10,000 to about 75,000, about 10,000 to about 70,000, about 10,000 to about 65,000, about 10,000 to about 60,000, about 10,000 to about 55,000, about 10,000 to about 50,000, about 10,000 to about 45,000, about 10,000 to about 40,000, about 10,000 to about 35,000, about 10,000 to about 30,000, about 10,000 to about 25,000, about 10,000 to about 20,000, about 10,000 to about 15,000, about 15,000 to about 10,000,000, about 15,000 to about 9,000,000, about 15,000 to about 8,000,000, about 15,000 to about 7,000,000, about 15,000 to about 6,000,000, about 15,000 to about 5,000,000, about 15,000 to about 4,000,000, about 15,000 to about 3,000,000, about 15,000 to about 2,000,000, about 15,000 to about 1,000,000, about 15,000 to about 800,000, about 15,000 to about 600,000, about 15,000 to about 400,000, about 15,000 to about 200,000, about 15,000 to about 100,000, about 15,000 to about 80,000, about 15,000 to about 75,000, about 15,000 to about 70,000, about 15,000 to about 65,000, about 15,000 to about 60,000, about 15,000 to about 55,000, about 15,000 to about 50,000, about 15,000 to about 45,000, about 15,000 to about 40,000, about 15,000 to about 35,000, about 15,000 to about 30,000, about 15,000 to about 25,000, about 15,000 to about 20,000, about 20,000 to about 10,000,000, about 20,000 to about 9,000,000, about 20,000 to about 8,000,000, about 20,000 to about 7,000,000, about 20,000 to about 6,000,000, about 20,000 to about 5,000,000, about 20,000 to about 4,000,000, about 20,000 to about 3,000,000, about 20,000 to about 2,000,000, about 20,000 to about 1,000,000, about 20,000 to about 800,000, about 20,000 to about 600,000, about 20,000 to about 400,000, about 20,000 to about 200,000, about 20,000 to about 100,000, about 20,000 to about 80,000, about 20,000 to about 75,000, about 20,000 to about 70,000, about 20,000 to about 65,000, about 210,000 to about 60,000, about 20,000 to about 55,000, about 20,000 to about 50,000, about 20,000 to about 45,000, about 20,000 to about 40,000, about 20,000 to about 35,000, about 20,000 to about 30,000, about 20,000 to about 25,000, about 25,000 to about 10,000,000, about 25,000 to about 9,000,000, about 25,000 to about 8,000,000, about 25,000 to about 7,000,000, about 25,000 to about 6,000,000, about 25,000 to about 5,000,000, about 25,000 to about 4,000,000, about 25,000 to about 3,000,000, about 25,000 to about 2,000,000, about 25,000 to about 1,000,000, about 25,000 to about 800,000, about 25,000 to about 600,000, about 25,000 to about 400,000, about 25,000 to about 200,000, about 25,000 to about 100,000, about 25,000 to about 80,000, about 25,000 to about 75,000, about 25,000 to about 70,000, about 25,000 to about 65,000, about 25,000 to about 60,000, about 25,000 to about 55,000, about 25,000 to about 50,000, about 25,000 to about 45,000, about 25,000 to about 40,000, about 25,000 to about 35,000, about 25,000 to about 30,000, about 30,000 to about 10,000,000, about 30,000 to about 9,000,000, about 30,000 to about 8,000,000, about 30,000 to about 7,000,000, about 30,000 to about 6,000,000, about 30,000 to about 5,000,000, about 30,000 to about 4,000,000, about 30,000 to about 3,000,000, about 30,000 to about 2,000,000, about 30,000 to about 1,000,000, about 30,000 to about 800,000, about 30,000 to about 600,000, about 30,000 to about 400,000, about 30,000 to about 200,000, about 30,000 to about 100,000, about 30,000 to about 80,000, about 30,000 to about 75,000, about 30,000 to about 70,000, about 30,000 to about 65,000, about 30,000 to about 60,000, about 30,000 to about 55,000, about 30,000 to about 50,000, about 30,000 to about 45,000, about 30,000 to about 40,000, about 30,000 to about 35,000, about 35,000 to about 10,000,000, about 35,000 to about 9,000,000, about 35,000 to about 8,000,000, about 35,000 to about 7,000,000, about 35,000 to about 6,000,000, about 35,000 to about 5,000,000, about 35,000 to about 4,000,000, about 35,000 to about 3,000,000, about 35,000 to about 2,000,000, about 35,000 to about 1,000,000, about 35,000 to about 800,000, about 35,000 to about 600,000, about 35,000 to about 400,000, about 35,000 to about 200,000, about 35,000 to about 100,000, about 35,000 to about 80,000, about 35,000 to about 75,000, about 35,000 to about 70,000, about 35,000 to about 65,000, about 35,000 to about 60,000, about 35,000 to about 55,000, about 35,000 to about 50,000, about 35,000 to about 45,000, about 35,000 to about 40,000, about 40,000 to about 10,000,000, about 40,000 to about 9,000,000, about 40,000 to about 8,000,000, about 40,000 to about 7,000,000, about 40,000 to about 6,000,000, about 40,000 to about 5,000,000, about 40,000 to about 4,000,000, about 40,000 to about 3,000,000, about 40,000 to about 2,000,000, about 40,000 to about 1,000,000, about 40,000 to about 800,000, about 40,000 to about 600,000, about 40,000 to about 400,000, about 40,000 to about 200,000, about 40,000 to about 100,000, about 40,000 to about 80,000, about 40,000 to about 75,000, about 40,000 to about 70,000, about 40,000 to about 65,000, about 40,000 to about 60,000, about 40,000 to about 55,000, about 40,000 to about 50,000, about 40,000 to about 45,000, about 45,000 to about 10,000,000, about 45,000 to about 9,000,000, about 45,000 to about 8,000,000, about 45,000 to about 7,000,000, about 45,000 to about 6,000,000, about 45,000 to about 5,000,000, about 45,000 to about 4,000,000, about 45,000 to about 3,000,000, about 45,000 to about 2,000,000, about 45,000 to about 1,000,000, about 45,000 to about 800,000, about 45,000 to about 600,000, about 45,000 to about 400,000, about 45,000 to about 200,000, about 45,000 to about 100,000, about 45,000 to about 80,000, about 45,000 to about 75,000, about 45,000 to about 70,000, about 45,000 to about 65,000, about 45,000 to about 60,000, about 45,000 to about 55,000, about 45,000 to about 50,000, about 50,000 to about 10,000,000, about 50,000 to about 9,000,000, about 50,000 to about 8,000,000, about 50,000 to about 7,000,000, about 50,000 to about 6,000,000, about 50,000 to about 5,000,000, about 50,000 to about 4,000,000, about 50,000 to about 3,000,000, about 50,000 to about 2,000,000, about 50,000 to about 1,000,000, about 50,000 to about 800,000, about 50,000 to about 600,000, about 50,000 to about 400,000, about 50,000 to about 200,000, about 50,000 to about 100,000, about 50,000 to about 80,000, about 50,000 to about 75,000, about 50,000 to about 70,000, about 50,000 to about 65,000, about 50,000 to about 60,000, about 50,000 to about 55,000, about 55,000 to about 10,000,000, about 55,000 to about 9,000,000, about 55,000 to about 8,000,000, about 55,000 to about 7,000,000, about 55,000 to about 6,000,000, about 55,000 to about 5,000,000, about 55,000 to about 4,000,000, about 55,000 to about 3,000,000, about 55,000 to about 2,000,000, about 55,000 to about 1,000,000, about 55,000 to about 800,000, about 55,000 to about 600,000, about 55,000 to about 400,000, about 55,000 to about 200,000, about 55,000 to about 100,000, about 55,000 to about 80,000, about 55,000 to about 75,000, about 55,000 to about 70,000, about 55,000 to about 65,000, about 55,000 to about 60,000, about 60,000 to about 10,000,000, about 60,000 to about 9,000,000, about 60,000 to about 8,000,000, about 60,000 to about 7,000,000, about 60,000 to about 6,000,000, about 60,000 to about 5,000,000, about 60,000 to about 4,000,000, about 60,000 to about 3,000,000, about 60,000 to about 2,000,000, about 60,000 to about 1,000,000, about 60,000 to about 800,000, about 60,000 to about 600,000, about 60,000 to about 400,000, about 60,000 to about 200,000, about 60,000 to about 100,000, about 60,000 to about 80,000, about 60,000 to about 75,000, about 60,000 to about 70,000, about 60,000 to about 65,000, about 65,000 to about 10,000,000, about 65,000 to about 9,000,000, about 65,000 to about 8,000,000, about 65,000 to about 7,000,000, about 65,000 to about 6,000,000, about 65,000 to about 5,000,000, about 65,000 to about 4,000,000, about 65,000 to about 3,000,000, about 65,000 to about 2,000,000, about 65,000 to about 1,000,000, about 65,000 to about 800,000, about 65,000 to about 600,000, about 65,000 to about 400,000, about 65,000 to about 200,000, about 65,000 to about 100,000, about 65,000 to about 80,000, about 65,000 to about 75,000, about 65,000 to about 70,000, about 70,000 to about 10,000,000, about 70,000 to about 9,000,000, about 70,000 to about 8,000,000, about 70,000 to about 7,000,000, about 70,000 to about 6,000,000, about 70,000 to about 5,000,000, about 70,000 to about 4,000,000, about 70,000 to about 3,000,000, about 70,000 to about 2,000,000, about 70,000 to about 1,000,000, about 70,000 to about 800,000, about 70,000 to about 600,000, about 70,000 to about 400,000, about 70,000 to about 200,000, about 70,000 to about 100,000, about 70,000 to about 90,000, about 70,000 to about 80,000, about 80,000 to about 10,000,000, about 80,000 to about 9,000,000, about 80,000 to about 8,000,000, about 80,000 to about 7,000,000, about 80,000 to about 6,000,000, about 80,000 to about 5,000,000, about 80,000 to about 4,000,000, about 80,000 to about 3,000,000, about 80,000 to about 2,000,000, about 80,000 to about 1,000,000, about 80,000 to about 800,000, about 80,000 to about 600,000, about 80,000 to about 400,000, about 80,000 to about 200,000, about 80,000 to about 100,000, about 80,000 to about 90,000, about 90,000 to about 10,000,000, about 90,000 to about 9,000,000, about 90,000 to about 8,000,000, about 90,000 to about 7,000,000, about 90,000 to about 6,000,000, about 90,000 to about 5,000,000, about 90,000 to about 4,000,000, about 90,000 to about 3,000,000, about 90,000 to about 2,000,000, about 90,000 to about 1,000,000, about 90,000 to about 800,000, about 90,000 to about 600,000, about 90,000 to about 400,000, about 90,000 to about 200,000, about 90,000 to about 100,000, about 100,000 to about 10,000,000, about 100,000 to about 9,000,000, about 100,000 to about 8,000,000, about 100,000 to about 7,000,000, about 100,000 to about 6,000,000, about 100,000 to about 5,000,000, about 100,000 to about 4,000,000, about 100,000 to about 3,000,000, about 100,000 to about 2,000,000, about 100,000 to about 1,000,000, about 100,000 to about 800,000, about 100,000 to about 600,000, about 100,000 to about 400,000, about 100,000 to about 200,000, about 200,000 to about 10,000,000, about 200,000 to about 9,000,000, about 200,000 to about 8,000,000, about 200,000 to about 7,000,000, about 200,000 to about 6,000,000, about 200,000 to about 5,000,000, about 200,000 to about 4,000,000, about 200,000 to about 3,000,000, about 200,000 to about 2,000,000, about 200,000 to about 1,000,000, about 200,000 to about 800,000, about 200,000 to about 600,000, about 200,000 to about 400,000, about 400,000 to about 10,000,000, about 400,000 to about 9,000,000, about 400,000 to about 8,000,000, about 400,000 to about 7,000,000, about 400,000 to about 6,000,000, about 400,000 to about 5,000,000, about 400,000 to about 4,000,000, about 400,000 to about 3,000,000, about 400,000 to about 2,000,000, about 400,000 to about 1,000,000, about 400,000 to about 800,000, about 400,000 to about 600,000, about 600,000 to about 10,000,000, about 600,000 to about 9,000,000, about 600,000 to about 8,000,000, about 600,000 to about 7,000,000, about 600,000 to about 6,000,000, about 600,000 to about 5,000,000, about 600,000 to about 4,000,000, about 600,000 to about 3,000,000, about 600,000 to about 2,000,000, about 600,000 to about 1,000,000, about 600,000 to about 800,000, about 800,000 to about 10,000,000, about 800,000 to about 9,000,000, about 800,000 to about 8,000,000, about 800,000 to about 7,000,000, about 800,000 to about 6,000,000, about 800,000 to about 5,000,000, about 800,000 to about 4,000,000, about 800,000 to about 3,000,000, about 800,000 to about 2,000,000, about 800,000 to about 1,000,000, about 1,000,000 to about 10,000,000, about 1,000,000 to about 9,000,000, about 1,000,000 to about 8,000,000, about 1,000,000 to about 7,000,000, about 1,000,000 to about 6,000,000, about 1,000,000 to about 5,000,000, about 1,000,000 to about 4,000,000, about 1,000,000 to about 3,000,000, about 1,000,000 to about 2,000,000, about 2,000,000 to about 10,000,000, about 2,000,000 to about 9,000,000, about 2,000,000 to about 8,000,000, about 2,000,000 to about 7,000,000, about 2,000,000 to about 6,000,000, about 2,000,000 to about 5,000,000, about 2,000,000 to about 4,000,000, about 2,000,000 to about 3,000,000, about 3,000,000 to about 10,000,000, about 3,000,000 to about 9,000,000, about 3,000,000 to about 8,000,000, about 3,000,000 to about 7,000,000, about 3,000,000 to about 6,000,000, about 3,000,000 to about 5,000,000, about 3,000,000 to about 4,000,000, about 4,000,000 to about 10,000,000, about 4,000,000 to about 9,000,000, about 4,000,000 to about 8,000,000, about 4,000,000 to about 7,000,000, about 4,000,000 to about 6,000,000, about 4,000,000 to about 5,000,000, about 5,000,000 to about 10,000,000, about 5,000,000 to about 9,000,000, about 5,000,000 to about 8,000,000, about 5,000,000 to about 7,000,000, about 5,000,000 to about 6,000,000, about 6,000,000 to about 10,000,000, about 6,000,000 to about 9,000,000, about 6,000,000 to about 8,000,000, about 6,000,000 to about 7,000,000, about 7,000,000 to about 10,000,000, about 7,000,000 to about 9,000,000, about 7,000,000 to about 8,000,000, about 8,000,000 to about 10,000,000, about 8,000,000 to about 9,000,000, or about 9,000,000 to about 10,000,000 of the DLL3 on present on the plasma membrane of the target mammalian cell.

The phrase “antigen density” means the number of DLL3 present on the surface of a target mammalian cell or the average number of DLL3 on the surface of a population of particular type of target mammalian cells. It can be measured, e.g., using the Quantibright bead kit or radiolabel (e.g., BD Biosciences PE Phycoerythrin Fluorescence Quantitation Kit, catalog #340495).

The phrase “amino acid substituted with a histidine” means the substitution of an amino acid residue that is not histidine in a reference polypeptide sequence with a histidine. Non-limiting methods for substituting an amino acid residue in a reference polypeptide with a histidine are described herein. Additional methods for substituting an amino acid residue in a reference polypeptide with a histidine are known in the art.

The phrase “amino acid substituted with an alanine” means the substitution of an amino acid residue that is a histidine in a reference polypeptide sequence with an alanine. Non-limiting methods for substituting a histidine in a reference polypeptide with an alanine are described herein. Additional methods for substituting a histidine in a reference polypeptide with an alanine are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1: SDS PAGE for rovalpituzumab histidine scanning and alanine scanning. Expi293 cell culture supernatants post-harvest were loaded on non-reduced SDS PAGE gels to confirm expression of rovalpituzumab and histidine scanning and alanine scanning variants. Arrows show the corresponding size for an IgG on a non-reduced SDS PAGE gel. MYT0638 is rovalpituzumab and the rest of the lanes (MYT0639-MYT0679) are rovalpituzumab heavy chain histidine scanning and alanine scanning variants.

FIG. 2: SDS PAGE for rovalpituzumab histidine scanning and alanine scanning. Expi293 cell culture supernatants post-harvest were loaded on non-reduced SDS PAGE gels to confirm expression of ROVALPITUZUMAB and histidine scanning and alanine scanning variants. Arrows show the corresponding size for an IgG on a non-reduced SDS PAGE gel. MYT0638 is ROVALPITUZUMAB and the rest of the lanes (MYT1532-MYT1558) are ROVALPITUZUMAB light chain histidine scanning and alanine scanning variants.

FIG. 3: SDS PAGE for ROVALPITUZUMAB histidine scanning and alanine scanning. Expi293 cell culture supernatants post-harvest were loaded on non-reduced SDS PAGE gels to confirm expression of ROVALPITUZUMAB and histidine scanning and alanine scanning variants. Arrows show the corresponding size for an IgG on a non-reduced SDS PAGE gel. MYT1183, MYT1186, MYT1187, MYT1188, MYT1189, MYT1190, MYT1192, MYT1198, MYT1199, MYT1200, and MYT1201 are ROVALPITUZUMAB heavy chain combinations histidine scanning and alanine scanning variants.

FIGS. 4ai to 4aq: Binding of rovalpituzumab starting ABPC and histidine scanning and alanine scanning variants to DLL3 by biolayer interferometry. MYT0638 (rovalpituzumab) and MYT639-MYT0679, heavy chain histidine scanning and alanine scanning variants, were captured on anti-human Fc biosensors and associated with DLL3 at low pH or high pH, as specified in the figures. Some figures show one or more flat curve(s), which represent reference conditions where no antibody was loaded on the sensor.

FIGS. 5a to 5ac: Binding of ROVALPITUZUMAB starting ABPC and histidine scanning and alanine scanning variants to DLL3 by biolayer interferometry. MYT0638 (rovalpituzumab) and MYT1532-MYT1558, light chain histidine scanning and alanine scanning variants, were captured on anti-human Fc biosensors and associated with DLL3 at low pH or high pH, as specified in the figures. Some figures show one or more flat curve(s), which represent reference conditions where no antibody was loaded on the sensor.

FIGS. 6a-6q: Binding of ROVALPITUZUMAB starting ABPC and histidine scanning and alanine scanning variants to DLL3 by biolayer interferometry. MYT0638 (rovalpituzumab), and MYT0645, MYT0647, MYT0656, MYT0677 (heavy chain histidine scanning and alanine scanning variants), and MYT1183, MYT1186, MYT1187, MYT1188, MYT1189, MYT1190, MYT1192, MYT1198, MYT1199, MYT1200, and MYT1201 (heavy chain combinations histidine scanning and alanine scanning variants) were captured on anti-human Fc biosensors and associated with DLL3 at low pH or high pH, as specified in the figures. Some figures show one or more flat curve(s), which represent reference conditions where no antibody was loaded on the sensor.

FIG. 7: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain, light chain, and CDR categories along the top.

FIG. 8: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain, light chain, and CDR categories along the top.

FIG. 9: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain, light chain, and CDR categories along the top.

FIG. 10: Construct identifier to SEQ ID NO correspondence table. Constructs, light chain combination histidine scanning and alanine scanning variants, are listed in the first column of the table, SEQ ID NOs are listed and correspond to constructs on the left and the appropriate heavy chain, light chain, and CDR categories along the top.

FIG. 11: Construct identifier to SEQ ID NO correspondence table. Constructs, paired heavy and light chain variants, combining light chain histidine and alanine scanning variants with heavy chain histidine and alanine scanning variants or heavy chain combination histidine scanning and alanine scanning variants, are listed in the first column of the table, SEQ ID NOs are listed and correspond to constructs on the left and the appropriate heavy chain, light chain, and CDR categories along the top.

FIG. 12: SDS PAGE for ROVALPITUZUMAB histidine scanning and alanine scanning. Expi293 cell culture supernatants post-harvest were loaded on non-reduced SDS PAGE gels to confirm expression of ROVALPITUZUMAB histidine scanning and alanine scanning variants. Arrows show the corresponding size for an IgG on a non-reduced SDS PAGE gel. MYT4262-MYT4281 are ROVALPITUZUMAB light chain combinations histidine scanning and alanine scanning variants.

FIGS. 13a to 13t: Binding of histidine scanning and alanine scanning variants of ROVALPITUZUMAB to DLL3 by biolayer interferometry. MYT4262-MYT4281, light chain combination histidine scanning and alanine scanning variants, were captured on anti-human Fc biosensors and associated with DLL3 at low pH or high pH, as specified in the figures.

FIG. 14: SDS PAGE for ROVALPITUZUMAB histidine scanning and alanine scanning. Expi293 cell culture supernatants post-harvest were loaded on non-reduced SDS PAGE gels to confirm expression of ROVALPITUZUMAB histidine scanning and alanine scanning variants. Arrows show the corresponding size for an IgG on a non-reduced SDS PAGE gel. MYT2800-MYT2845 are ROVALPITUZUMAB paired heavy and light chain variants, combining light chain histidine and alanine scanning variants with heavy chain histidine and alanine scanning variants or heavy chain combination histidine scanning and alanine scanning variants.

FIGS. 15a to 15at: Binding of histidine scanning and alanine scanning variants of ROVALPITUZUMAB to DLL3 by biolayer interferometry. MYT2800-MYT2845, paired heavy and light chain variants, combining light chain histidine and alanine scanning variants with heavy chain histidine and alanine scanning variants or heavy chain combination histidine scanning and alanine scanning variants, were captured on anti-human Fc biosensors and associated with DLL3 at low pH or high pH, as specified in the figures.

FIGS. 16a-j: Internalization of anti-DLL3 mAbs in NCI-1182 cells. Anti-DLL3 pH engineered antibody variants, corresponding starting ABPC antibodies, control IgG1 isotype control (BP0297, Bioxcell), along with a vehicle control, as specified in FIGS. 16a-j were assayed for internalization and endolysosomal delivery as measured by mean fluorescence intensity on NCI-H82 cells at 24 hours. Error bars represent standard deviation. Numbers above the bars represent fold change over wild-type.

FIGS. 17a-b: SDS PAGE for anti-DLL3 VH-Fc and VH-Fc converted to-IgG molecules. Expi293 cell culture supernatants post-harvest were loaded on non-reduced SDS PAGE gels to confirm expression of VH-Fc and VH-Fc converted to IgG. Arrows show the corresponding size for VH-Fc and IgG on a non-reduced SDS PAGE gel. MYT1044, MYT1047, MYT1048, MYT1050, MYT1052, MYT1053, MYT1054, MYT1055, MYT1056, MYT1057, MYT1059, MYT1060, MYT1061, MYT1062, MYT1063 are VH-Fc and MYT4373-4384 are IgG.

FIGS. 18a-ab: Binding of anti-DLL3 VH-Fc and VH-Fc converted to IgG molecules to DLL3 by biolayer interferometry. Rovalpituzumab (MYT0638), and MYT1044, MYT1047, MYT1048, MYT1050, MYT1052, MYT1053, MYT1054, MYT1055, MYT1056, MYT1057, MYT1059, MYT1060, MYT1061, MYT1062, MYT1063, VH-Fc molecules, and MYT4373-4384, IgG molecules, were captured on anti-human Fc biosensors and associated with DLL3 at low pH or high pH, as specified in the figures.

FIG. 19: Epitope binning of anti-DLL3 VH-Fc molecules. Biolayer interferometry was used to map the epitopes of anti-DLL3 VH-Fc molecules through competition with antibodies of known epitopes. The columns represent the different antibodies of a known epitope used to capture DLL3 while the rows represent the different VH-Fc constructs whose epitopes were being investigated. An X represents observed competition as measured by a greater than 30% decrease in binding to DLL3 when captured by a known antibody as compared to the same sample when measured against the maximum extent of binding obtained with any other known antibodies.

FIGS. 20a-c: Characterization of binding affinity for anti-DLL3 mAbs. Anti-DLL3 mAbs were assayed for their binding to NCI-H82 cells. FIG. 20a shows ROVALPITUZUMAB with an IC50 of 0.008 nM, FIG. 20b shows MYT2829 with an IC50 of 0.015 nM, FIG. 20c shows MYT1193 with an IC50 of 0.185 nM.

FIGS. 21a-d: Melting temperature of select anti-DLL3 mAbs. Anti-DLL3 mAbs were assayed for their melting temperature by Differential Scanning Flourimetry (DSF), and the resulting Sypro Orange signal was plotted as its first derivative. Melting temperature (Tm) was calculated as the local maxima of this graph. FIG. 21a; ROVALPITUZUMAB, Tm 69.5° C. FIG. 21b; MYT1193, Tm 59.9° C., 69.1° C. FIG. 21c; MYT1185, Tm 58.3° C., 69.1° C. FIG. 21d: MYT2829, Tm 67.9° C.

FIG. 22: Specificity of internalization and endolysosomal delivery of anti-DLL3 mAbs. ROVALPITUZUMAB, MYT1185, MYT1193, MYT1209, and MYT2829, control IgG1 isotype control (BP0297, Bioxcell), along with a vehicle control (“unstained”) were assayed for cell internalization and endolysosomal delivery as measured by mean fluorescence intensity on Raji cells (DLL3-) at 24 h. Errors bars represent standard deviation.

FIG. 23: SDS PAGE for SC16.4 histidine scanning and alanine scanning. Expi293 cell culture supernatants post-harvest were loaded on non-reduced SDS PAGE gels to confirm expression of SC16.4 and histidine scanning and alanine scanning variants. Arrows show the corresponding size for an IgG on a non-reduced SDS PAGE gel. MYT3047 is SC16.4 and the rest of the lanes (MYT3048-MYT3084) are SC16.4 heavy chain histidine scanning and alanine scanning variants.

FIGS. 24a to 24al: Binding of SC16.4 starting ABPC and histidine scanning and alanine scanning variants to DLL3 by biolayer interferometry. MYT3047 (SC16.4) and MYT3048-MYT3084, heavy chain histidine scanning and alanine scanning variants, were captured on anti-human Fc biosensors and associated with DLL3 at low pH or high pH, as specified in the figures.

FIG. 25: SDS PAGE for SC16.4 histidine scanning and alanine scanning. Expi293 cell culture supernatants post-harvest were loaded on non-reduced SDS PAGE gels to confirm expression of SC16.4 and histidine scanning and alanine scanning variants. Arrows show the corresponding size for an IgG on a non-reduced SDS PAGE gel. MYT3047 is SC16.4 and the rest of the lanes (MYT3085-MYT3110, MYT4200) are SC16.4 light chain histidine scanning and alanine scanning variants.

FIGS. 26a to 26aa: Binding of SC16.4 starting ABPC and histidine scanning and alanine scanning variants to DLL3 by biolayer interferometry. MYT3047 (SC16.4) and MYT3085-MYT3110, MYT4200, light chain histidine scanning and alanine scanning variants, were captured on anti-human Fc biosensors and associated with DLL3 at low pH or high pH, as specified in the figures.

FIG. 27: SDS PAGE for SC16.4 histidine scanning and alanine scanning. Expi293 cell culture supernatants post-harvest were loaded on non-reduced SDS PAGE gels to confirm expression of SC16.4 histidine scanning and alanine scanning variants. Arrows show the corresponding size for an IgG on a non-reduced SDS PAGE gel. MYT3399-MYT3424 and MYT4175-MYT4189 are SC16.4 heavy chain combinations histidine scanning and alanine scanning variants.

FIGS. 28a to 28ao: Binding of histidine scanning and alanine scanning variants of SC16.4 to DLL3 by biolayer interferometry. MYT3399-MYT3424 and MYT4175-MYT4189, heavy chain combination histidine scanning and alanine scanning variants, were captured on anti-human Fc biosensors and associated with DLL3 at low pH or high pH, as specified in the figures.

FIG. 29: SDS PAGE for SC16.4 histidine scanning and alanine scanning. Expi293 cell culture supernatants post-harvest were loaded on non-reduced SDS PAGE gels to confirm expression of SC16.4 histidine scanning and alanine scanning variants. Arrows show the corresponding size for an IgG on a non-reduced SDS PAGE gel. MYT4190-MYT4199 are SC16.4 light chain combinations histidine scanning and alanine scanning variants.

FIGS. 30a to 30j: Binding of histidine scanning and alanine scanning variants of SC16.4 to DLL3 by biolayer interferometry. MYT4190-MYT4199, light chain combination histidine scanning and alanine scanning variants, were captured on anti-human Fc biosensors and associated with DLL3 at low pH or high pH, as specified in the figures.

FIGS. 31: SDS PAGE for SC16.4 histidine scanning and alanine scanning. Expi293 cell culture supernatants post-harvest were loaded on non-reduced SDS PAGE gels to confirm expression of SC16.4 histidine scanning and alanine scanning variants. Arrows show the corresponding size for an IgG on a non-reduced SDS PAGE gel. MYT3493-MYT3513 are SC16.4 paired heavy and light chain variants, combining light chain histidine and alanine scanning variants with heavy chain histidine and alanine scanning variants FIGS. 32a to 32u: Binding of histidine scanning and alanine scanning variants of SC16.4 to DLL3 by biolayer interferometry. MYT3493-MYT3513, paired heavy and light chain variants, combining light chain histidine and alanine scanning variants with heavy chain histidine and alanine scanning variants were captured on anti-human Fc biosensors and associated with DLL3 at low pH or high pH, as specified in the figures.

FIG. 33: SDS PAGE for hSC16.13 histidine scanning and alanine scanning. Expi293 cell culture supernatants post-harvest were loaded on non-reduced SDS PAGE gels to confirm expression of hSC16.13 and histidine scanning and alanine scanning variants. Arrows show the corresponding size for an IgG on a non-reduced SDS PAGE gel. MYT3181 is hSC16.13 and the rest of the lanes (MYT3182-MYT3225) are hSC16.13 heavy chain histidine scanning and alanine scanning variants.

FIGS. 34a to 34as: Binding of hSC16.13 starting ABPC and histidine scanning and alanine scanning variants to DLL3 by biolayer interferometry. MYT3181 (hSC16.13) and MYT3182-MYT3225, heavy chain histidine scanning and alanine scanning variants, were captured on anti-human Fc biosensors and associated with DLL3 at low pH or high pH, as specified in the figures.

FIG. 35: SDS PAGE for hSC16.13 histidine scanning and alanine scanning. Expi293 cell culture supernatants post-harvest were loaded on non-reduced SDS PAGE gels to confirm expression of hSC16.13 and histidine scanning and alanine scanning variants. Arrows show the corresponding size for an IgG on a non-reduced SDS PAGE gel. MYT3226-MYT3251 are hSC16.13 light chain histidine scanning and alanine scanning variants.

FIGS. 36a to 36z: Binding of hSC16.13 starting ABPC and histidine scanning and alanine scanning variants to DLL3 by biolayer interferometry. MYT3181 (hSC16.13) and MYT3226-MYT3251, light chain histidine scanning and alanine scanning variants, were captured on anti-human Fc biosensors and associated with DLL3 at low pH or high pH, as specified in the figures.

FIG. 37: SDS PAGE for hSC16.13 histidine scanning and alanine scanning. Expi293 cell culture supernatants post-harvest were loaded on non-reduced SDS PAGE gels to confirm expression of hSC16.13 histidine scanning and alanine scanning variants. Arrows show the corresponding size for an IgG on a non-reduced SDS PAGE gel. MYT4346-MYT4371 are hSC16.13 heavy chain combinations histidine scanning and alanine scanning variants.

FIGS. 38a to 38z: Binding of histidine scanning and alanine scanning variants of hSC16.13 to DLL3 by biolayer interferometry. MYT4346-MYT4371, heavy chain combination histidine scanning and alanine scanning variants, were captured on anti-human Fc biosensors and associated with DLL3 at low pH or high pH, as specified in the figures.

FIG. 39: SDS PAGE for hSC16.13 histidine scanning and alanine scanning. Expi293 cell culture supernatants post-harvest were loaded on non-reduced SDS PAGE gels to confirm expression of a hSC16.13 histidine scanning and alanine scanning variant. Arrows show the corresponding size for an IgG on a non-reduced SDS PAGE gel. MYT4372 is a hSC16.13 light chain combinations histidine scanning and alanine scanning variant.

FIG. 40: Binding of histidine scanning and alanine scanning variants of hSC16.13 to DLL3 by biolayer interferometry. MYT4372, a light chain combination histidine scanning and alanine scanning variant, was captured on anti-human Fc biosensors and associated with DLL3 at low pH or high pH, as specified in the figures.

FIG. 41: SDS PAGE for hSC16.13 histidine scanning and alanine scanning. Expi293 cell culture supernatants post-harvest were loaded on non-reduced SDS PAGE gels to confirm expression of hSC16.13 histidine scanning and alanine scanning variants. Arrows show the corresponding size for an IgG on a non-reduced SDS PAGE gel. MYT3526-MYT3551 are hSC16.13 paired heavy and light chain variants, combining light chain histidine and alanine scanning variants with heavy chain histidine and alanine scanning variants.

FIGS. 42a to 42z: Binding of histidine scanning and alanine scanning variants of HSC16.13 to DLL3 by biolayer interferometry. MYT3526-MYT3551, paired heavy and light chain variants, combining light chain histidine and alanine scanning variants with heavy chain histidine and alanine scanning variants, were captured on anti-human Fc biosensors and associated with DLL3 at low pH or high pH, as specified in the figures.

FIG. 43: SDS PAGE for hSC16.15 histidine scanning and alanine scanning. Expi293 cell culture supernatants post-harvest were loaded on non-reduced SDS PAGE gels to confirm expression of hSC16.15 and histidine scanning and alanine scanning variants. Arrows show the corresponding size for an IgG on a non-reduced SDS PAGE gel. MYT3892 is hSC16.15 and the rest of the lanes (MYT3893-MYT3932) are hSC16.15 heavy chain histidine scanning and alanine scanning variants.

FIGS. 44a to 44ao: Binding of hSC16.15 starting ABPC and histidine scanning and alanine scanning variants to DLL3 by biolayer interferometry. MYT3892 (hSC16.15) and MYT3893-MYT3932, heavy chain histidine scanning and alanine scanning variants, were captured on anti-human Fc biosensors and associated with DLL3 at low pH or high pH, as specified in the figures.

FIG. 45: SDS PAGE for hSC16.15 histidine scanning and alanine scanning. Expi293 cell culture supernatants post-harvest were loaded on non-reduced SDS PAGE gels to confirm expression of hSC16.15 and histidine scanning and alanine scanning variants. Arrows show the corresponding size for an IgG on a non-reduced SDS PAGE gel. MYT3933-MYT3956 are hSC16.15 light chain histidine scanning and alanine scanning variants.

FIGS. 46a to 46x: Binding of hSC16.15 starting ABPC and histidine scanning and alanine scanning variants to DLL3 by biolayer interferometry. MYT3933-MYT3956, light chain histidine scanning and alanine scanning variants, were captured on anti-human Fc biosensors and associated with DLL3 at low pH or high pH, as specified in the figures.

FIG. 47: SDS PAGE for hSC16.25 histidine scanning and alanine scanning. Expi293 cell culture supernatants post-harvest were loaded on non-reduced SDS PAGE gels to confirm expression of hSC16.25 and histidine scanning and alanine scanning variants. Arrows show the corresponding size for an IgG on a non-reduced SDS PAGE gel. MYT3758 is hSC16.25 and the rest of the lanes (MYT3759-MYT3802) are hSC16.25 heavy chain histidine scanning and alanine scanning variants.

FIGS. 48a to 48as: Binding of hSC16.25 starting ABPC and histidine scanning and alanine scanning variants to DLL3 by biolayer interferometry. MYT3758 (hSC16.25) and MYT3759-MYT3802, heavy chain histidine scanning and alanine scanning variants, were captured on anti-human Fc biosensors and associated with DLL3 at low pH or high pH, as specified in the figures.

FIG. 49: SDS PAGE for hSC16.25 histidine scanning and alanine scanning. Expi293 cell culture supernatants post-harvest were loaded on non-reduced SDS PAGE gels to confirm expression of hSC16.25 and histidine scanning and alanine scanning variants. Arrows show the corresponding size for an IgG on a non-reduced SDS PAGE gel. MYT3803-MYT3825 are hSC16.25 light chain histidine scanning and alanine scanning variants.

FIGS. 50a to 50w: Binding of hSC16.25 starting ABPC and histidine scanning and alanine scanning variants to DLL3 by biolayer interferometry. MYT3803-MYT3825, light chain histidine scanning and alanine scanning variants, were captured on anti-human Fc biosensors and associated with DLL3 at low pH or high pH, as specified in the figures.

FIG. 51: SDS PAGE for hSC16.34 histidine scanning and alanine scanning. Expi293 cell culture supernatants post-harvest were loaded on non-reduced SDS PAGE gels to confirm expression of hSC16.34 and histidine scanning and alanine scanning variants. Arrows show the corresponding size for an IgG on a non-reduced SDS PAGE gel. MYT3826 is hSC16.34 and the rest of the lanes (MYT3827-MYT3867) are hSC16.34 heavy chain histidine scanning and alanine scanning variants.

FIGS. 52a to 52ap: Binding of hSC16.34 starting ABPC and histidine scanning and alanine scanning variants to DLL3 by biolayer interferometry. MYT3826 (hSC16.34) and MYT3827-MYT3867, heavy chain histidine scanning and alanine scanning variants, were captured on anti-human Fc biosensors and associated with DLL3 at low pH or high pH, as specified in the figures.

FIG. 53: SDS PAGE for hSC16.34 histidine scanning and alanine scanning. Expi293 cell culture supernatants post-harvest were loaded on non-reduced SDS PAGE gels to confirm expression of hSC16.34 and histidine scanning and alanine scanning variants. Arrows show the corresponding size for an IgG on a non-reduced SDS PAGE gel. MYT3868-MYT3891 are hSC16.34 light chain histidine scanning and alanine scanning variants.

FIGS. 54a to 54x: Binding of hSC16.34 starting ABPC and histidine scanning and alanine scanning variants to DLL3 by biolayer interferometry. MYT3868-MYT3891, light chain histidine scanning and alanine scanning variants, were captured on anti-human Fc biosensors and associated with DLL3 at low pH or high pH, as specified in the figures.

FIG. 55: SDS PAGE for SC16.67 histidine scanning and alanine scanning. Expi293 cell culture supernatants post-harvest were loaded on non-reduced SDS PAGE gels to confirm expression of SC16.67 and histidine scanning and alanine scanning variants. Arrows show the corresponding size for an IgG on a non-reduced SDS PAGE gel. MYT3111 is SC16.67 and the rest of the lanes (MYT3112-MYT3148) are SC16.67 heavy chain histidine scanning and alanine scanning variants.

FIGS. 56a to 56al: Binding of SC16.67 starting ABPC and histidine scanning and alanine scanning variants to DLL3 by biolayer interferometry. MYT3111 (SC16.67) and MYT3112-MYT3148, heavy chain histidine scanning and alanine scanning variants, were captured on anti-human Fc biosensors and associated with DLL3 at low pH or high pH, as specified in the figures.

FIG. 57: SDS PAGE for SC16.67 histidine scanning and alanine scanning. Expi293 cell culture supernatants post-harvest were loaded on non-reduced SDS PAGE gels to confirm expression of SC16.67 and histidine scanning and alanine scanning variants. Arrows show the corresponding size for an IgG on a non-reduced SDS PAGE gel. MYT3149-MYT3178 are SC16.67 light chain histidine scanning and alanine scanning variants.

FIGS. 58a to 58ad: Binding of SC16.67 starting ABPC and histidine scanning and alanine scanning variants to DLL3 by biolayer interferometry. MYT3149-MYT3178, light chain histidine scanning and alanine scanning variants, were captured on anti-human Fc biosensors and associated with DLL3 at low pH or high pH, as specified in the figures.

FIG. 59: SDS PAGE for SC16.67 histidine scanning and alanine scanning. Expi293 cell culture supernatants post-harvest were loaded on non-reduced SDS PAGE gels to confirm expression of SC16.67 histidine scanning and alanine scanning variants. Arrows show the corresponding size for an IgG on a non-reduced SDS PAGE gel. MYT3514-MYT3525 are SC16.67 paired heavy and light chain variants, combining light chain histidine and alanine scanning variants with heavy chain histidine and alanine scanning variants.

FIGS. 60a to 60l: Binding of histidine scanning and alanine scanning variants of SC16.67 to DLL3 by biolayer interferometry. MYT3514-MYT3525, paired heavy and light chain variants, combining light chain histidine and alanine scanning variants with heavy chain histidine and alanine scanning variants, were captured on anti-human Fc biosensors and associated with DLL3 at low pH or high pH, as specified in the figures.

FIG. 61: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 62: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 63: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain and/or light chain categories along the top.

FIG. 64: Construct identifier to SEQ ID NO correspondence table. Constructs, light chain combinations histidine scanning and alanine scanning variants, are listed in the first column of the table, SEQ ID NOs are listed and correspond to constructs on the left and the appropriate heavy chain and/or light chain categories along the top.

FIG. 65: Construct identifier to SEQ ID NO correspondence table. Constructs, paired heavy and light chain variants, combining light chain histidine and alanine scanning variants with heavy chain histidine and alanine scanning variants are listed in the first column of the table, SEQ ID NOs are listed and correspond to constructs on the left and the appropriate heavy chain and/or light chain categories along the top.

FIG. 66: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 67: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 68: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain and/or light chain categories along the top.

FIG. 69: Construct identifier to SEQ ID NO correspondence table. Constructs, light chain combinations histidine scanning and alanine scanning variants, are listed in the first column of the table, SEQ ID NOs are listed and correspond to constructs on the left and the appropriate heavy chain and/or light chain categories along the top.

FIG. 70: Construct identifier to SEQ ID NO correspondence table. Constructs, paired heavy and light chain variants, combining light chain histidine and alanine scanning variants with heavy chain histidine and alanine scanning variants are listed in the first column of the table, SEQ ID NOs are listed and correspond to constructs on the left and the appropriate heavy chain and/or light chain categories along the top.

FIG. 71: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 72: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 73: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 74: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 75: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 76: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 77: Construct identifier to SEQ ID NO correspondence table. Constructs, heavy chain histidine scanning and alanine scanning variants, are listed in the first column of the table, SEQ ID NOs are listed and correspond to constructs on the left and the appropriate heavy chain and/or light chain categories along the top.

FIG. 78: Construct identifier to SEQ ID NO correspondence table. Constructs, light chain histidine scanning and alanine scanning variants, are listed in the first column of the table, SEQ ID NOs are listed and correspond to constructs on the left and the appropriate heavy chain and/or light chain categories along the top.

FIG. 79: Construct identifier to SEQ ID NO correspondence table. Constructs, paired heavy and light chain variants, combining light chain histidine and alanine scanning variants with heavy chain histidine and alanine scanning variants are listed in the first column of the table, SEQ ID NOs are listed and correspond to constructs on the left and the appropriate heavy chain and/or light chain categories along the top.

FIG. 80: Construct identifier to SEQ ID NO correspondence table. Constructs, VH-Fc and VH-Fc converted to IgG, are listed in the first column of the table, SEQ ID NOs are listed and correspond to constructs on the left and the appropriate heavy chain and/or light chain categories along the top.

FIG. 81: Construct identifier to SEQ ID NO correspondence table. Constructs, heavy chain combinations histidine scanning and alanine scanning variants, are listed in the first column of the table, SEQ ID NOs are listed and correspond to constructs on the left and the appropriate heavy chain and/or light chain categories along the top.

FIG. 82: SDS PAGE for ROVALPITUZUMAB histidine scanning and alanine scanning. Expi293 cell culture supernatants post-harvest were loaded on non-reduced SDS PAGE gels to confirm expression of ROVALPITUZUMAB histidine scanning and alanine scanning variants. Arrows show the corresponding size for an IgG on a non-reduced SDS PAGE gel. MYT1182, MYT1183, MYT1185, MYT1193, MYT1194, MYT1195, and MYT1196 are ROVALPITUZUMAB heavy chain combinations histidine scanning and alanine scanning variants.

FIGS. 83a to 83g: Binding of histidine scanning and alanine scanning variants of ROVALPITUZUMAB to DLL3 by biolayer interferometry. MYT1182, MYT1183, MYT1185, MYT1193, MYT1194, MYT1195, and MYT1196, heavy chain combination histidine scanning and alanine scanning variants, were captured on anti-human Fc biosensors and associated with DLL3 at low pH or high pH, as specified in the figures.

FIG. 84: Construct identifier to SEQ ID NO correspondence table. Constructs, starting ABPCs, are listed in the first column of the table, SEQ ID NOs are listed and correspond to constructs on the left and the appropriate CDR categories along the top.

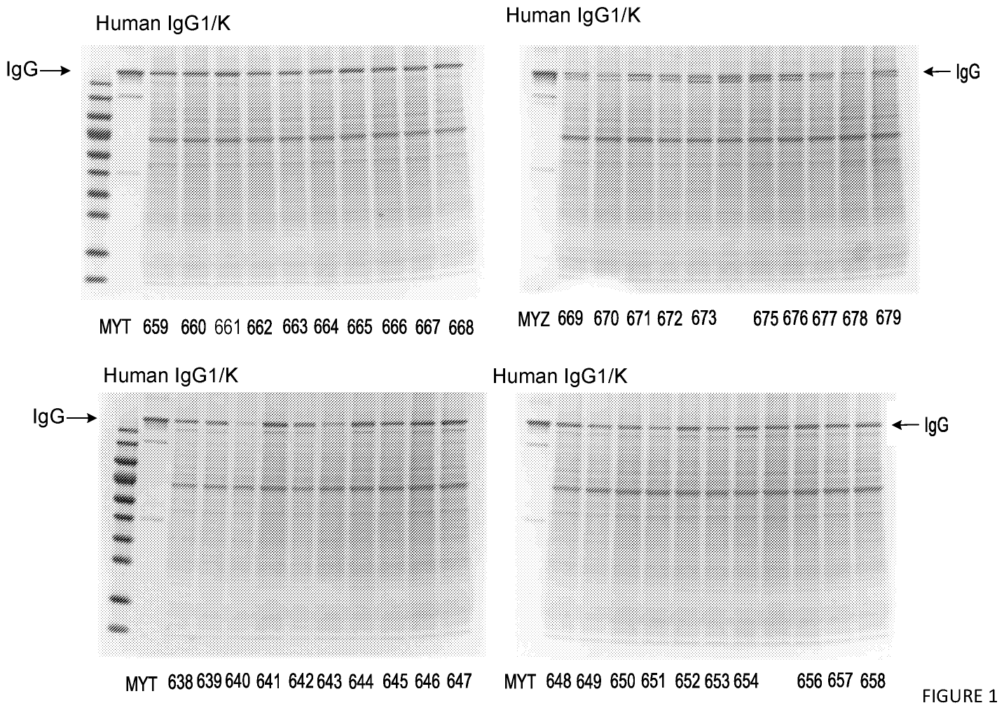

### DETAILED DESCRIPTION

Provided herein are antigen-binding protein constructs (ABPCs) that include: a first antigen-binding domain that is capable of specifically binding DLL3 or an epitope of DLL3 presented on the surface of a target mammalian cell, where: (a) the dissociation rate of the first antigen-binding domain at a pH of about 4.0 to about 6.5 is faster than the dissociation rate at a pH of about 7.0 to about 8.0; and/or (b) the dissociation constant (K_{D}) of the first antigen-binding domain at a pH of about 4.0 to about 6.5 is greater than the K_{D} at a pH of about 7.0 to about 8.0. In some examples of these ABPCs, the ABPC is degraded in the target mammalian cell following internalization of the ABPC by the target mammalian cell. Some examples of any of the ABPCs described herein can further include a conjugated toxin, radioisotope, drug, or small molecule (e.g., a fluorophore or dye).

Also provided are antigen-binding protein constructs (ABPCs) that include: a first antigen-binding domain that is capable of specifically binding DLL3 or an epitope of DLL3 presented on the surface of a target mammalian cell; and a conjugated toxin, radioisotope, drug, or small molecule, where: (a) the dissociation rate of the first antigen-binding domain at a pH of about 4.0 to about 6.5 is faster than the dissociation rate at a pH of about 7.0 to about 8.0; and/or the dissociation constant (K_{D}) of the first antigen-binding domain at a pH of about 4.0 to about 6.5 is greater than the K_{D} at a pH of about 7.0 to about 8.0; and (b) a composition including the ABPC provides for one or more (e.g., two or three) of: an increase (e.g., a detectable increase) in toxin liberation in the target mammalian cell as compared to a composition comprising the same amount of a control ABPC; an increase (e.g., a detectable increase) in target mammalian cell killing as compared to a composition comprising the same amount of a control ABPC; and an increase (e.g., a detectable increase) in endolysosomal delivery in the target mammalian cell as compared to a composition comprising the same amount of a control ABPC.

In some examples of any of the ABPCs described herein, the first antigen-binding domain includes a heavy chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of rovalpituzumab comprises SEQ ID NO: 1. In some examples of any of the ABPCs described herein, the light chain variable domain of rovalpituzumab comprises SEQ ID NO: 2.

In some examples of any of the ABPCs described herein, the first antigen-binding domain comprises a heavy chain variable domain comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) amino acid positions in SEQ ID NOs: 3-5 substituted with a histidine. In some examples of any of the ABPCs described herein, the first antigen-binding domain comprises a light chain variable domain comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) amino acid positions in SEQ ID NOs: 6-8 substituted with a histidine. In some examples of any of the ABPCs described herein, the first antigen-binding domain includes: a heavy chain variable domain comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) amino acid positions in SEQ ID NOs: 3-5 substituted with a histidine; and a light chain variable domain comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) amino acid positions in SEQ ID NOs: 6-8 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 1 selected from the group consisting of: 27, 29, 31, 32, 34, 35, 50, 53, 54, 55, 58, 97, 98, 101, 103, 105, and 106. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 2, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 2 selected from the group consisting of: 25, 26, 29, 32, 33, 34, 51, 54, 89, 90, 93, 94, 95, and 96. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 1 selected from the group consisting of: 27, 29, 31, 32, 34, 35, 50, 53, 54, 55, 58, 97, 98, 101, 103, 105, and 106, and a light chain variable domain that is at least 90% (e.g., at least 92%, at least 94%, at least 96%, at least 98%, at least 99%, or 100%) identical to SEQ ID NO: 2, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 2 selected from the group consisting of: 25, 26, 29, 32, 33, 34, 51, 54, 89, 90, 93, 94, 95, and 96.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

TABLE 1
Exemplary Combinations of Amino Acid Positions in
SEQ ID NO: 1 that can be Substituted with Histidine

29 + 27, 31 + 27, 32 + 27, 34 + 27, 35 + 27, 50 + 27, 53 + 27, 54 + 27, 55 + 27, 58 + 27, 97 +
27, 98 + 27, 101 + 27, 103 + 27, 105 + 27, 106 + 27, 31 + 29, 32 + 29, 34 + 29, 35 + 29, 50 +
29, 53 + 29, 54 + 29, 55 + 29, 58 + 29, 97 + 29, 98 + 29, 101 + 29, 103 + 29, 105 + 29, 106 +
29, 32 + 31, 34 + 31, 35 + 31, 50 + 31, 53 + 31, 54 + 31, 55 + 31, 58 + 31, 97 + 31, 98 + 31,
101 + 31, 103 + 31, 105 + 31, 106 + 31, 34 + 32, 35 + 32, 50 + 32, 53 + 32, 54 + 32, 55 + 32,
58 + 32, 97 + 32, 98 + 32, 101 + 32, 103 + 32, 105 + 32, 106 + 32, 35 + 34, 50 + 34, 53 + 34,
54 + 34, 55 + 34, 58 + 34, 97 + 34, 98 + 34, 101 + 34, 103 + 34, 105 + 34, 106 + 34, 50 + 35,
53 + 35, 54 + 35, 55 + 35, 58 + 35, 97 + 35, 98 + 35, 101 + 35, 103 + 35, 105 + 35, 106 + 35,
53 + 50, 54 + 50, 55 + 50, 58 + 50, 97 + 50, 98 + 50, 101 + 50, 103 + 50, 105 + 50, 106 + 50,
54 + 53, 55 + 53, 58 + 53, 97 + 53, 98 + 53, 101 + 53, 103 + 53, 105 + 53, 106 + 53, 55 + 54,
58 + 54, 97 + 54, 98 + 54, 101 + 54, 103 + 54, 105 + 54, 106 + 54, 58 + 55, 97 + 55, 98 + 55,
101 + 55, 103 + 55, 105 + 55, 106 + 55, 97 + 58, 98 + 58, 101 + 58, 103 + 58, 105 + 58, 106 +
58, 98 + 97, 101 + 97, 103 + 97, 105 + 97, 106 + 97, 101 + 98, 103 + 98, 105 + 98, 106 + 98,
103 + 101, 105 + 101, 106 + 101, 105 + 103, 106 + 103, 106 + 105, 27 + 31 + 29, 27 + 32 +
29, 27 + 34 + 29, 27 + 35 + 29, 27 + 50 + 29, 27 + 53 + 29, 27 + 54 + 29, 27 + 55 + 29, 27 +
58 + 29, 27 + 97 + 29, 27 + 98 + 29, 27 + 101 + 29, 27 + 103 + 29, 27 + 105 + 29, 27 + 106 +
29, 27 + 32 + 31, 27 + 34 + 31, 27 + 35 + 31, 27 + 50 + 31, 27 + 53 + 31, 27 + 54 + 31, 27 +
55 + 31, 27 + 58 + 31, 27 + 97 + 31, 27 + 98 + 31, 27 + 101 + 31, 27 + 103 + 31, 27 + 105 +
31, 27 + 106 + 31, 29 + 32 + 31, 29 + 34 + 31, 29 + 35 + 31, 29 + 50 + 31, 29 + 53 + 31, 29 +
54 + 31, 29 + 55 + 31, 29 + 58 + 31, 29 + 97 + 31, 29 + 98 + 31, 29 + 101 + 31, 29 + 103 +
31, 29 + 105 + 31, 29 + 106 + 31, 27 + 34 + 32, 27 + 35 + 32, 27 + 50 + 32, 27 + 53 + 32, 27 +
54 + 32, 27 + 55 + 32, 27 + 58 + 32, 27 + 97 + 32, 27 + 98 + 32, 27 + 101 + 32, 27 + 103 +
32, 27 + 105 + 32, 27 + 106 + 32, 29 + 34 + 32, 29 + 35 + 32, 29 + 50 + 32, 29 + 53 + 32, 29 +
54 + 32, 29 + 55 + 32, 29 + 58 + 32, 29 + 97 + 32, 29 + 98 + 32, 29 + 101 + 32, 29 + 103 +
32, 29 + 105 + 32, 29 + 106 + 32, 31 + 34 + 32, 31 + 35 + 32, 31 + 50 + 32, 31 + 53 + 32, 31 +
54 + 32, 31 + 55 + 32, 31 + 58 + 32, 31 + 97 + 32, 31 + 98 + 32, 31 + 101 + 32, 31 + 103 +
32, 31 + 105 + 32, 31 + 106 + 32, 27 + 35 + 34, 27 + 50 + 34, 27 + 53 + 34, 27 + 54 + 34, 27 +
55 + 34, 27 + 58 + 34, 27 + 97 + 34, 27 + 98 + 34, 27 + 101 + 34, 27 + 103 + 34, 27 + 105 +
34, 27 + 106 + 34, 29 + 35 + 34, 29 + 50 + 34, 29 + 53 + 34, 29 + 54 + 34, 29 + 55 + 34, 29 +
58 + 34, 29 + 97 + 34, 29 + 98 + 34, 29 + 101 + 34, 29 + 103 + 34, 29 + 105 + 34, 29 + 106 +
34, 31 + 35 + 34, 31 + 50 + 34, 31 + 53 + 34, 31 + 54 + 34, 31 + 55 + 34, 31 + 58 + 34, 31 +
97 + 34, 31 + 98 + 34, 31 + 101 + 34, 31 + 103 + 34, 31 + 105 + 34, 31 + 106 + 34, 32 + 35 +
34, 32 + 50 + 34, 32 + 53 + 34, 32 + 54 + 34, 32 + 55 + 34, 32 + 58 + 34, 32 + 97 + 34, 32 +
98 + 34, 32 + 101 + 34, 32 + 103 + 34, 32 + 105 + 34, 32 + 106 + 34, 27 + 50 + 35, 27 + 53 +
35, 27 + 54 + 35, 27 + 55 + 35, 27 + 58 + 35, 27 + 97 + 35, 27 + 98 + 35, 27 + 101 + 35, 27 +
103 + 35, 27 + 105 + 35, 27 + 106 + 35, 29 + 50 + 35, 29 + 53 + 35, 29 + 54 + 35, 29 + 55 +
35, 29 + 58 + 35, 29 + 97 + 35, 29 + 98 + 35, 29 + 101 + 35, 29 + 103 + 35, 29 + 105 + 35, 29 +
106 + 35, 31 + 50 + 35, 31 + 53 + 35, 31 + 54 + 35, 31 + 55 + 35, 31 + 58 + 35, 31 + 97 +
35, 31 + 98 + 35, 31 + 101 + 35, 31 + 103 + 35, 31 + 105 + 35, 31 + 106 + 35, 32 + 50 + 35,
32 + 53 + 35, 32 + 54 + 35, 32 + 55 + 35, 32 + 58 + 35, 32 + 97 + 35, 32 + 98 + 35, 32 + 101 +
35, 32 + 103 + 35, 32 + 105 + 35, 32 + 106 + 35, 34 + 50 + 35, 34 + 53 + 35, 34 + 54 + 35,
34 + 55 + 35, 34 + 58 + 35, 34 + 97 + 35, 34 + 98 + 35, 34 + 101 + 35, 34 + 103 + 35, 34 +
105 + 35, 34 + 106 + 35, 27 + 53 + 50, 27 + 54 + 50, 27 + 55 + 50, 27 + 58 + 50, 27 + 97 +
50, 27 + 98 + 50, 27 + 101 + 50, 27 + 103 + 50, 27 + 105 + 50, 27 + 106 + 50, 29 + 53 + 50,
29 + 54 + 50, 29 + 55 + 50, 29 + 58 + 50, 29 + 97 + 50, 29 + 98 + 50, 29 + 101 + 50, 29 + 103 +
50, 29 + 105 + 50, 29 + 106 + 50, 31 + 53 + 50, 31 + 54 + 50, 31 + 55 + 50, 31 + 58 + 50,
31 + 97 + 50, 31 + 98 + 50, 31 + 101 + 50, 31 + 103 + 50, 31 + 105 + 50, 31 + 106 + 50, 32 +
53 + 50, 32 + 54 + 50, 32 + 55 + 50, 32 + 58 + 50, 32 + 97 + 50, 32 + 98 + 50, 32 + 101 + 50,
32 + 103 + 50, 32 + 105 + 50, 32 + 106 + 50, 34 + 53 + 50, 34 + 54 + 50, 34 + 55 + 50, 34 +
58 + 50, 34 + 97 + 50, 34 + 98 + 50, 34 + 101 + 50, 34 + 103 + 50, 34 + 105 + 50, 34 + 106 +
50, 35 + 53 + 50, 35 + 54 + 50, 35 + 55 + 50, 35 + 58 + 50, 35 + 97 + 50, 35 + 98 + 50, 35 +
101 + 50, 35 + 103 + 50, 35 + 105 + 50, 35 + 106 + 50, 27 + 54 + 53, 27 + 55 + 53, 27 + 58 +
53, 27 + 97 + 53, 27 + 98 + 53, 27 + 101 + 53, 27 + 103 + 53, 27 + 105 + 53, 27 + 106 + 53,
29 + 54 + 53, 29 + 55 + 53, 29 + 58 + 53, 29 + 97 + 53, 29 + 98 + 53, 29 + 101 + 53, 29 + 103 +
53, 29 + 105 + 53, 29 + 106 + 53, 31 + 54 + 53, 31 + 55 + 53, 31 + 58 + 53, 31 + 97 + 53,
31 + 98 + 53, 31 + 101 + 53, 31 + 103 + 53, 31 + 105 + 53, 31 + 106 + 53, 32 + 54 + 53, 32 +
55 + 53, 32 + 58 + 53, 32 + 97 + 53, 32 + 98 + 53, 32 + 101 + 53, 32 + 103 + 53, 32 + 105 +
53, 32 + 106 + 53, 34 + 54 + 53, 34 + 55 + 53, 34 + 58 + 53, 34 + 97 + 53, 34 + 98 + 53, 34 +
101 + 53, 34 + 103 + 53, 34 + 105 + 53, 34 + 106 + 53, 35 + 54 + 53, 35 + 55 + 53, 35 + 58 +
53, 35 + 97 + 53, 35 + 98 + 53, 35 + 101 + 53, 35 + 103 + 53, 35 + 105 + 53, 35 + 106 + 53,
50 + 54 + 53, 50 + 55 + 53, 50 + 58 + 53, 50 + 97 + 53, 50 + 98 + 53, 50 + 101 + 53, 50 + 103 +
53, 50 + 105 + 53, 50 + 106 + 53, 27 + 55 + 54, 27 + 58 + 54, 27 + 97 + 54, 27 + 98 + 54,
27 + 101 + 54, 27 + 103 + 54, 27 + 105 + 54, 27 + 106 + 54, 29 + 55 + 54, 29 + 58 + 54, 29 +
97 + 54, 29 + 98 + 54, 29 + 101 + 54, 29 + 103 + 54, 29 + 105 + 54, 29 + 106 + 54, 31 + 55 +
54, 31 + 58 + 54, 31 + 97 + 54, 31 + 98 + 54, 31 + 101 + 54, 31 + 103 + 54, 31 + 105 + 54, 31 +
106 + 54, 32 + 55 + 54, 32 + 58 + 54, 32 + 97 + 54, 32 + 98 + 54, 32 + 101 + 54, 32 + 103 +
54, 32 + 105 + 54, 32 + 106 + 54, 34 + 55 + 54, 34 + 58 + 54, 34 + 97 + 54, 34 + 98 + 54, 34 +
101 + 54, 34 + 103 + 54, 34 + 105 + 54, 34 + 106 + 54, 35 + 55 + 54, 35 + 58 + 54, 35 + 97 +
54, 35 + 98 + 54, 35 + 101 + 54, 35 + 103 + 54, 35 + 105 + 54, 35 + 106 + 54, 50 + 55 + 54,
50 + 58 + 54, 50 + 97 + 54, 50 + 98 + 54, 50 + 101 + 54, 50 + 103 + 54, 50 + 105 + 54, 50 +
106 + 54, 53 + 55 + 54, 53 + 58 + 54, 53 + 97 + 54, 53 + 98 + 54, 53 + 101 + 54, 53 + 103 +
54, 53 + 105 + 54, 53 + 106 + 54, 27 + 58 + 55, 27 + 97 + 55, 27 + 98 + 55, 27 + 101 + 55, 27 +
103 + 55, 27 + 105 + 55, 27 + 106 + 55, 29 + 58 + 55, 29 + 97 + 55, 29 + 98 + 55, 29 + 101 +
55, 29 + 103 + 55, 29 + 105 + 55, 29 + 106 + 55, 31 + 58 + 55, 31 + 97 + 55, 31 + 98 + 55,
31 + 101 + 55, 31 + 103 + 55, 31 + 105 + 55, 31 + 106 + 55, 32 + 58 + 55, 32 + 97 + 55, 32 +
98 + 55, 32 + 101 + 55, 32 + 103 + 55, 32 + 105 + 55, 32 + 106 + 55, 34 + 58 + 55, 34 + 97 +
55, 34 + 98 + 55, 34 + 101 + 55, 34 + 103 + 55, 34 + 105 + 55, 34 + 106 + 55, 35 + 58 + 55,
35 + 97 + 55, 35 + 98 + 55, 35 + 101 + 55, 35 + 103 + 55, 35 + 105 + 55, 35 + 106 + 55, 50 +
58 + 55, 50 + 97 + 55, 50 + 98 + 55, 50 + 101 + 55, 50 + 103 + 55, 50 + 105 + 55, 50 + 106 +
55, 53 + 58 + 55, 53 + 97 + 55, 53 + 98 + 55, 53 + 101 + 55, 53 + 103 + 55, 53 + 105 + 55, 53 +
106 + 55, 54 + 58 + 55, 54 + 97 + 55, 54 + 98 + 55, 54 + 101 + 55, 54 + 103 + 55, 54 + 105 +
55, 54 + 106 + 55, 27 + 97 + 58, 27 + 98 + 58, 27 + 101 + 58, 27 + 103 + 58, 27 + 105 + 58,
27 + 106 + 58, 29 + 97 + 58, 29 + 98 + 58, 29 + 101 + 58, 29 + 103 + 58, 29 + 105 + 58, 29 +
106 + 58, 31 + 97 + 58, 31 + 98 + 58, 31 + 101 + 58, 31 + 103 + 58, 31 + 105 + 58, 31 + 106 +
58, 32 + 97 + 58, 32 + 98 + 58, 32 + 101 + 58, 32 + 103 + 58, 32 + 105 + 58, 32 + 106 + 58,
34 + 97 + 58, 34 + 98 + 58, 34 + 101 + 58, 34 + 103 + 58, 34 + 105 + 58, 34 + 106 + 58, 35 +
97 + 58, 35 + 98 + 58, 35 + 101 + 58, 35 + 103 + 58, 35 + 105 + 58, 35 + 106 + 58, 50 + 97 +
58, 50 + 98 + 58, 50 + 101 + 58, 50 + 103 + 58, 50 + 105 + 58, 50 + 106 + 58, 53 + 97 + 58,
53 + 98 + 58, 53 + 101 + 58, 53 + 103 + 58, 53 + 105 + 58, 53 + 106 + 58, 54 + 97 + 58, 54 +
98 + 58, 54 + 101 + 58, 54 + 103 + 58, 54 + 105 + 58, 54 + 106 + 58, 55 + 97 + 58, 55 + 98 +
58, 55 + 101 + 58, 55 + 103 + 58, 55 + 105 + 58, 55 + 106 + 58, 27 + 98 + 97, 27 + 101 + 97,
27 + 103 + 97, 27 + 105 + 97, 27 + 106 + 97, 29 + 98 + 97, 29 + 101 + 97, 29 + 103 + 97, 29 +
105 + 97, 29 + 106 + 97, 31 + 98 + 97, 31 + 101 + 97, 31 + 103 + 97, 31 + 105 + 97, 31 +
106 + 97, 32 + 98 + 97, 32 + 101 + 97, 32 + 103 + 97, 32 + 105 + 97, 32 + 106 + 97, 34 + 98 +
97, 34 + 101 + 97, 34 + 103 + 97, 34 + 105 + 97, 34 + 106 + 97, 35 + 98 + 97, 35 + 101 +
97, 35 + 103 + 97, 35 + 105 + 97, 35 + 106 + 97, 50 + 98 + 97, 50 + 101 + 97, 50 + 103 + 97,
50 + 105 + 97, 50 + 106 + 97, 53 + 98 + 97, 53 + 101 + 97, 53 + 103 + 97, 53 + 105 + 97, 53 +
106 + 97, 54 + 98 + 97, 54 + 101 + 97, 54 + 103 + 97, 54 + 105 + 97, 54 + 106 + 97, 55 +
98 + 97, 55 + 101 + 97, 55 + 103 + 97, 55 + 105 + 97, 55 + 106 + 97, 58 + 98 + 97, 58 + 101 +
97, 58 + 103 + 97, 58 + 105 + 97, 58 + 106 + 97, 27 + 101 + 98, 27 + 103 + 98, 27 + 105 +
98, 27 + 106 + 98, 29 + 101 + 98, 29 + 103 + 98, 29 + 105 + 98, 29 + 106 + 98, 31 + 101 + 98,
31 + 103 + 98, 31 + 105 + 98, 31 + 106 + 98, 32 + 101 + 98, 32 + 103 + 98, 32 + 105 + 98, 32 +
106 + 98, 34 + 101 + 98, 34 + 103 + 98, 34 + 105 + 98, 34 + 106 + 98, 35 + 101 + 98, 35 +
103 + 98, 35 + 105 + 98, 35 + 106 + 98, 50 + 101 + 98, 50 + 103 + 98, 50 + 105 + 98, 50 +
106 + 98, 53 + 101 + 98, 53 + 103 + 98, 53 + 105 + 98, 53 + 106 + 98, 54 + 101 + 98, 54 +
103 + 98, 54 + 105 + 98, 54 + 106 + 98, 55 + 101 + 98, 55 + 103 + 98, 55 + 105 + 98, 55 +
106 + 98, 58 + 101 + 98, 58 + 103 + 98, 58 + 105 + 98, 58 + 106 + 98, 97 + 101 + 98, 97 +
103 + 98, 97 + 105 + 98, 97 + 106 + 98, 27 + 103 + 101, 27 + 105 + 101, 27 + 106 + 101, 29 +
103 + 101, 29 + 105 + 101, 29 + 106 + 101, 31 + 103 + 101, 31 + 105 + 101, 31 + 106 + 101,
32 + 103 + 101, 32 + 105 + 101, 32 + 106 + 101, 34 + 103 + 101, 34 + 105 + 101, 34 + 106 +
101, 35 + 103 + 101, 35 + 105 + 101, 35 + 106 + 101, 50 + 103 + 101, 50 + 105 + 101, 50 +
106 + 101, 53 + 103 + 101, 53 + 105 + 101, 53 + 106 + 101, 54 + 103 + 101, 54 + 105 + 101,
54 + 106 + 101, 55 + 103 + 101, 55 + 105 + 101, 55 + 106 + 101, 58 + 103 + 101, 58 + 105 +
101, 58 + 106 + 101, 97 + 103 + 101, 97 + 105 + 101, 97 + 106 + 101, 98 + 103 + 101, 98 +
105 + 101, 98 + 106 + 101, 27 + 105 + 103, 27 + 106 + 103, 29 + 105 + 103, 29 + 106 + 103,
31 + 105 + 103, 31 + 106 + 103, 32 + 105 + 103, 32 + 106 + 103, 34 + 105 + 103, 34 + 106 +
103, 35 + 105 + 103, 35 + 106 + 103, 50 + 105 + 103, 50 + 106 + 103, 53 + 105 + 103, 53 +
106 + 103, 54 + 105 + 103, 54 + 106 + 103, 55 + 105 + 103, 55 + 106 + 103, 58 + 105 + 103,
58 + 106 + 103, 97 + 105 + 103, 97 + 106 + 103, 98 + 105 + 103, 98 + 106 + 103, 101 + 105 +
103, 101 + 106 + 103, 27 + 106 + 105, 29 + 106 + 105, 31 + 106 + 105, 32 + 106 + 105, 34 +
106 + 105, 35 + 106 + 105, 50 + 106 + 105, 53 + 106 + 105, 54 + 106 + 105, 55 + 106 + 105,
58 + 106 + 105, 97 + 106 + 105, 98 + 106 + 105, 101 + 106 + 105, 103 + 106 + 105

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 2, where the light chain variable domain includes a histidine at any of the specific combinations of one or more amino acid positions in SEQ ID NO: 2 listed in Table 2.

TABLE 2
Exemplary Combinations of Amino Acid Positions in
SEQ ID NO: 2 that can be Substituted with Histidine

25 + 26, 25 + 29, 25 + 32, 25 + 33, 25 + 34, 25 + 51, 25 + 54, 25 + 89, 25 + 90, 25 + 93, 25 +
94, 25 + 95, 25 + 96, 26 + 29, 26 + 32, 26 + 33, 26 + 34, 26 + 51, 26 + 54, 26 + 89, 26 + 90,
26 + 93, 26 + 94, 26 + 95, 26 + 96, 29 + 32, 29 + 33, 29 + 34, 29 + 51, 29 + 54, 29 + 89, 29 +
90, 29 + 93, 29 + 94, 29 + 95, 29 + 96, 32 + 33, 32 + 34, 32 + 51, 32 + 54, 32 + 89, 32 + 90,
32 + 93, 32 + 94, 32 + 95, 32 + 96, 33 + 34, 33 + 51, 33 + 54, 33 + 89, 33 + 90, 33 + 93, 33 +
94, 33 + 95, 33 + 96, 34 + 51, 34 + 54, 34 + 89, 34 + 90, 34 + 93, 34 + 94, 34 + 95, 34 + 96,
51 + 54, 51 + 89, 51 + 90, 51 + 93, 51 + 94, 51 + 95, 51 + 96, 54 + 89, 54 + 90, 54 + 93, 54 +
94, 54 + 95, 54 + 96, 89 + 90, 89 + 93, 89 + 94, 89 + 95, 89 + 96, 90 + 93, 90 + 94, 90 + 95,
90 + 96, 93 + 94, 93 + 95, 93 + 96, 94 + 95, 94 + 96, 95 + 96, 25 + 26 + 29, 25 + 26 + 32, 25 +
26 + 33, 25 + 26 + 34, 25 + 26 + 51, 25 + 26 + 54, 25 + 26 + 89, 25 + 26 + 90, 25 + 26 + 93,
25 + 26 + 94, 25 + 26 + 95, 25 + 26 + 96, 25 + 29 + 32, 25 + 29 + 33, 25 + 29 + 34, 25 + 29 +
51, 25 + 29 + 54, 25 + 29 + 89, 25 + 29 + 90, 25 + 29 + 93, 25 + 29 + 94, 25 + 29 + 95, 25 +
29 + 96, 26 + 29 + 32, 26 + 29 + 33, 26 + 29 + 34, 26 + 29 + 51, 26 + 29 + 54, 26 + 29 + 89,
26 + 29 + 90, 26 + 29 + 93, 26 + 29 + 94, 26 + 29 + 95, 26 + 29 + 96, 25 + 32 + 33, 25 + 32 +
34, 25 + 32 + 51, 25 + 32 + 54, 25 + 32 + 89, 25 + 32 + 90, 25 + 32 + 93, 25 + 32 + 94, 25 +
32 + 95, 25 + 32 + 96, 26 + 32 + 33, 26 + 32 + 34, 26 + 32 + 51, 26 + 32 + 54, 26 + 32 + 89,
26 + 32 + 90, 26 + 32 + 93, 26 + 32 + 94, 26 + 32 + 95, 26 + 32 + 96, 29 + 32 + 33, 29 + 32 +
34, 29 + 32 + 51, 29 + 32 + 54, 29 + 32 + 89, 29 + 32 + 90, 29 + 32 + 93, 29 + 32 + 94, 29 +
32 + 95, 29 + 32 + 96, 25 + 33 + 34, 25 + 33 + 51, 25 + 33 + 54, 25 + 33 + 89, 25 + 33 + 90,
25 + 33 + 93, 25 + 33 + 94, 25 + 33 + 95, 25 + 33 + 96, 26 + 33 + 34, 26 + 33 + 51, 26 + 33 +
54, 26 + 33 + 89, 26 + 33 + 90, 26 + 33 + 93, 26 + 33 + 94, 26 + 33 + 95, 26 + 33 + 96, 29 +
33 + 34, 29 + 33 + 51, 29 + 33 + 54, 29 + 33 + 89, 29 + 33 + 90, 29 + 33 + 93, 29 + 33 + 94,
29 + 33 + 95, 29 + 33 + 96, 32 + 33 + 34, 32 + 33 + 51, 32 + 33 + 54, 32 + 33 + 89, 32 + 33 +
90, 32 + 33 + 93, 32 + 33 + 94, 32 + 33 + 95, 32 + 33 + 96, 25 + 34 + 51, 25 + 34 + 54, 25 +
34 + 89, 25 + 34 + 90, 25 + 34 + 93, 25 + 34 + 94, 25 + 34 + 95, 25 + 34 + 96, 26 + 34 + 51,
26 + 34 + 54, 26 + 34 + 89, 26 + 34 + 90, 26 + 34 + 93, 26 + 34 + 94, 26 + 34 + 95, 26 + 34 +
96, 29 + 34 + 51, 29 + 34 + 54, 29 + 34 + 89, 29 + 34 + 90, 29 + 34 + 93, 29 + 34 + 94, 29 +
34 + 95, 29 + 34 + 96, 32 + 34 + 51, 32 + 34 + 54, 32 + 34 + 89, 32 + 34 + 90, 32 + 34 + 93,
32 + 34 + 94, 32 + 34 + 95, 32 + 34 + 96, 33 + 34 + 51, 33 + 34 + 54, 33 + 34 + 89, 33 + 34 +
90, 33 + 34 + 93, 33 + 34 + 94, 33 + 34 + 95, 33 + 34 + 96, 25 + 51 + 54, 25 + 51 + 89, 25 +
51 + 90, 25 + 51 + 93, 25 + 51 + 94, 25 + 51 + 95, 25 + 51 + 96, 26 + 51 + 54, 26 + 51 + 89,
26 + 51 + 90, 26 + 51 + 93, 26 + 51 + 94, 26 + 51 + 95, 26 + 51 + 96, 29 + 51 + 54, 29 + 51 +
89, 29 + 51 + 90, 29 + 51 + 93, 29 + 51 + 94, 29 + 51 + 95, 29 + 51 + 96, 32 + 51 + 54, 32 +
51 + 89, 32 + 51 + 90, 32 + 51 + 93, 32 + 51 + 94, 32 + 51 + 95, 32 + 51 + 96, 33 + 51 + 54,
33 + 51 + 89, 33 + 51 + 90, 33 + 51 + 93, 33 + 51 + 94, 33 + 51 + 95, 33 + 51 + 96, 34 + 51 +
54, 34 + 51 + 89, 34 + 51 + 90, 34 + 51 + 93, 34 + 51 + 94, 34 + 51 + 95, 34 + 51 + 96, 25 +
54 + 89, 25 + 54 + 90, 25 + 54 + 93, 25 + 54 + 94, 25 + 54 + 95, 25 + 54 + 96, 26 + 54 + 89,
26 + 54 + 90, 26 + 54 + 93, 26 + 54 + 94, 26 + 54 + 95, 26 + 54 + 96, 29 + 54 + 89, 29 + 54 +
90, 29 + 54 + 93, 29 + 54 + 94, 29 + 54 + 95, 29 + 54 + 96, 32 + 54 + 89, 32 + 54 + 90, 32 +
54 + 93, 32 + 54 + 94, 32 + 54 + 95, 32 + 54 + 96, 33 + 54 + 89, 33 + 54 + 90, 33 + 54 + 93,
33 + 54 + 94, 33 + 54 + 95, 33 + 54 + 96, 34 + 54 + 89, 34 + 54 + 90, 34 + 54 + 93, 34 + 54 +
94, 34 + 54 + 95, 34 + 54 + 96, 51 + 54 + 89, 51 + 54 + 90, 51 + 54 + 93, 51 + 54 + 94, 51 +
54 + 95, 51 + 54 + 96, 25 + 89 + 90, 25 + 89 + 93, 25 + 89 + 94, 25 + 89 + 95, 25 + 89 + 96,
26 + 89 + 90, 26 + 89 + 93, 26 + 89 + 94, 26 + 89 + 95, 26 + 89 + 96, 29 + 89 + 90, 29 + 89 +
93, 29 + 89 + 94, 29 + 89 + 95, 29 + 89 + 96, 32 + 89 + 90, 32 + 89 + 93, 32 + 89 + 94, 32 +
89 + 95, 32 + 89 + 96, 33 + 89 + 90, 33 + 89 + 93, 33 + 89 + 94, 33 + 89 + 95, 33 + 89 + 96,
34 + 89 + 90, 34 + 89 + 93, 34 + 89 + 94, 34 + 89 + 95, 34 + 89 + 96, 51 + 89 + 90, 51 + 89 +
93, 51 + 89 + 94, 51 + 89 + 95, 51 + 89 + 96, 54 + 89 + 90, 54 + 89 + 93, 54 + 89 + 94, 54 +
89 + 95, 54 + 89 + 96, 25 + 90 + 93, 25 + 90 + 94, 25 + 90 + 95, 25 + 90 + 96, 26 + 90 + 93,
26 + 90 + 94, 26 + 90 + 95, 26 + 90 + 96, 29 + 90 + 93, 29 + 90 + 94, 29 + 90 + 95, 29 + 90 +
96, 32 + 90 + 93, 32 + 90 + 94, 32 + 90 + 95, 32 + 90 + 96, 33 + 90 + 93, 33 + 90 + 94, 33 +
90 + 95, 33 + 90 + 96, 34 + 90 + 93, 34 + 90 + 94, 34 + 90 + 95, 34 + 90 + 96, 51 + 90 + 93,
51 + 90 + 94, 51 + 90 + 95, 51 + 90 + 96, 54 + 90 + 93, 54 + 90 + 94, 54 + 90 + 95, 54 + 90 +
96, 89 + 90 + 93, 89 + 90 + 94, 89 + 90 + 95, 89 + 90 + 96, 25 + 93 + 94, 25 + 93 + 95, 25 +
93 + 96, 26 + 93 + 94, 26 + 93 + 95, 26 + 93 + 96, 29 + 93 + 94, 29 + 93 + 95, 29 + 93 + 96,
32 + 93 + 94, 32 + 93 + 95, 32 + 93 + 96, 33 + 93 + 94, 33 + 93 + 95, 33 + 93 + 96, 34 + 93 +
94, 34 + 93 + 95, 34 + 93 + 96, 51 + 93 + 94, 51 + 93 + 95, 51 + 93 + 96, 54 + 93 + 94, 54 +
93 + 95, 54 + 93 + 96, 89 + 93 + 94, 89 + 93 + 95, 89 + 93 + 96, 90 + 93 + 94, 90 + 93 + 95,
90 + 93 + 96, 25 + 94 + 95, 25 + 94 + 96, 26 + 94 + 95, 26 + 94 + 96, 29 + 94 + 95, 29 + 94 +
96, 32 + 94 + 95, 32 + 94 + 96, 33 + 94 + 95, 33 + 94 + 96, 34 + 94 + 95, 34 + 94 + 96, 51 +
94 + 95, 51 + 94 + 96, 54 + 94 + 95, 54 + 94 + 96, 89 + 94 + 95, 89 + 94 + 96, 90 + 94 + 95,
90 + 94 + 96, 93 + 94 + 95, 93 + 94 + 96, 25 + 95 + 96, 26 + 95 + 96, 29 + 95 + 96, 32 + 95 +
96, 33 + 95 + 96, 34 + 95 + 96, 51 + 95 + 96, 54 + 95 + 96, 89 + 95 + 96, 90 + 95 + 96, 93 +
95 + 96, 94 + 95 + 96

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1; and a light chain variable domain that that is at least 90% (e.g., at least 92%, at least 94%, at least 96%, at least 98%, at least 99%, or 100%) identical to SEQ ID NO: 2, where the light chain variable domain includes a histidine at any of the specific combinations of one or more amino acid positions in SEQ ID NO: 2 listed in Table 2.

In some examples of any of the ABPCs described herein, the first antigen-binding domain comprises a light chain variable domain that is at least 90% identical to SEQ ID NO: 2, wherein the light chain variable domain includes a histidine at position 25 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain comprising SEQ ID NO: 2, and a heavy chain variable domain that is at least 90% identical (e.g., at least 92%, at least 94%, at least 96%, at least 98%, at least 99%, or 100%) identical to SEQ ID NO: 1, where the heavy chain variable domain includes a histidine at any of the specific combinations of one or more (e.g., two, three, four, five, six, seven, eight, nine, or ten) amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 26 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 29 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 32 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 33 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 34 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 51 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 54 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 89 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 90 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 93 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 94 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 95 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain comprises a light chain variable domain that is at least 90% identical to SEQ ID NO: 2, wherein the light chain variable domain includes a histidine at position 96 in SEQ ID NO: 2; and a heavy chain variable domain that is at least 90% (e.g., at least 92%, at least 94%, at least 96%, at least 98%, at least 99%, or 100%) identical to SEQ ID NO: 1, where the heavy chain variable domain includes a histidine at any of the specific combinations of one or more amino acid positions in SEQ ID NO: 1 listed in Table 1.

In some examples of any of the ABPCs described herein, the first antigen-binding domain includes a heavy chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of rovalpituzumab with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of rovalpituzumab comprises SEQ ID NO: 1. In some examples of any of the ABPCs described herein, the light chain variable domain of rovalpituzumab comprises SEQ ID NO: 2.

In some examples of any of the ABPCs described herein, the first antigen-binding domain comprises a heavy chain variable domain comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) histidine(s) in SEQ ID NOs: 3-5 substituted with an alanine. In some examples of any of the ABPCs described herein, the first antigen-binding domain comprises a light chain variable domain comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) histidine(s) in SEQ ID NOs: 6-8 substituted with an alanine. In some examples of any of the ABPCs described herein, the first antigen-binding domain includes: a heavy chain variable domain comprising a CDR1, a CDR2, and a CDR3 of SEQ ID NO: 3, SEQ ID NO: 4, and SEQ ID NO: 5, respectively, with collectively a total of one or more (e.g., one, two, three, four, five, six, seven, eight, nine, or ten) histidine(s) in SEQ ID NOs: 3-5 substituted with an alanine; and a light chain variable domain comprising a CDR1, a CDR2, and a CDR3 of SEQ ID NO: 6, SEQ ID NO: 7, and SEQ ID NO: 8, respectively, with collectively a total of one or more (e.g., one, two, three, four, five, six, seven, eight, nine, or ten) histidine(s) in SEQ ID NOs: 6-8 substituted with an alanine.

In some examples of any of the ABPCs described herein, the first antigen-binding domain comprises a heavy chain variable domain of SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain of SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a light chain variable domain comprising SEQ ID NO: 2, and a heavy chain variable domain comprising SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 54, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 55, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 56, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 57, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 58, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 59, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 60, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 61, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 62, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 63, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 64, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 65, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 66, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 67, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 68, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 69, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 70, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 71, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 72, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 73, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 74, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 75, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 76, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 77, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 78, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 79, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a light chain variable domain comprising SEQ ID NO: 80, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In some examples of any of the ABPCs described herein, the first antigen-binding domain includes a heavy chain variable domain of SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98. In some examples of any of the ABPCs described herein, the first antigen-binding domain includes a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 81, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 82, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80 In some examples of any of the ABPCs described herein, the first antigen-binding domain includes a heavy chain variable domain comprising SEQ ID NO: 83, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80 In some examples of any of the ABPCs described herein, the first antigen-binding domain includes a heavy chain variable domain comprising SEQ ID NO: 84, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 85, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 86, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 87, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 88, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 89, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 90, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80.

In some examples of any of the ABPCs described herein, the first antigen-binding domain includes a heavy chain variable domain comprising SEQ ID NO: 91, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 92, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 93, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 95, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 96, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 97, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80.

In some examples of any of the ABPCs described herein, the first antigen-binding domain includes a heavy chain variable domain comprising SEQ ID NO: 98, and a light chain variable domain comprising SEQ ID NO: 2, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, or SEQ ID NO: 80. In some examples of any of the ABPCs described herein, the first antigen-binding domain includes a light chain variable domain comprising SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, or SEQ ID NO: 118.

In some examples of any of the ABPCs described herein, the first antigen-binding domain includes a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 99, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 100, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 101, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 102, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 103, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 104, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 105, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 106, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 107, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 108, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 109, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 110, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 111, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 112, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 113, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 114, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 114, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 115, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 116, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 117, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 118, and a heavy chain variable domain comprising SEQ ID NO: 1, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 81, SEQ ID NO: 92, SEQ ID NO: 93, SEQ ID NO: 82, SEQ ID NO: 83, SEQ ID NO: 84, SEQ ID NO: 85, SEQ ID NO: 86, SEQ ID NO: 87, SEQ ID NO: 95, SEQ ID NO: 96, SEQ ID NO: 97, SEQ ID NO: 98, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 65 or SEQ ID NO: 77.

In some examples of any of the ABPCs described herein, the first antigen-binding domain includes a heavy chain variable domain comprising SEQ ID NO: 19, SEQ ID NO: 21, SEQ ID NO: 30, SEQ ID NO: 31, SEQ ID NO: 51, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 65, and a heavy chain variable domain comprising SEQ ID NO: 19, SEQ ID NO: 21, SEQ ID NO: 30, SEQ ID NO: 31, SEQ ID NO: 51, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a light chain variable domain comprising SEQ ID NO: 77, and a heavy chain variable domain comprising SEQ ID NO: 19, SEQ ID NO: 21, SEQ ID NO: 30, SEQ ID NO: 31, SEQ ID NO: 51, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98.

In some examples of any of the ABPCs described herein, the first antigen-binding domain includes a heavy chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.4 comprises SEQ ID NO: 119. In some examples of any of the ABPCs described herein, the light chain variable domain of SC16.4 comprises SEQ ID NO: 120.

In some examples of any of the ABPCs described herein, the first antigen-binding domain comprises a heavy chain variable domain comprising a CDR1, a CDR2, and a CDR3 of SEQ ID NO: 622, SEQ ID NO: 623, and SEQ ID NO: 624, respectively, with collectively a total of one or more (e.g., one, two, three, four, five, six, seven, eight, nine, or ten) amino acid positions in SEQ ID NOs: 622-624 substituted with a histidine. In some examples of any of the ABPCs described herein, the first antigen-binding domain comprises a light chain variable domain comprising a CDR1, a CDR2, and a CDR3 of SEQ ID NO: 625, SEQ ID NO: 626, and SEQ ID NO: 627, respectively, with collectively a total of one or more (e.g., one, two, three, four, five, six, seven, eight, nine, or ten) amino acid positions in SEQ ID NOs: 625-627 substituted with a histidine. In some examples of any of the ABPCs described herein, the first antigen-binding domain includes: a heavy chain variable domain comprising a CDR1, a CDR2, and a CDR3 of SEQ ID NO: 622, SEQ ID NO: 623, and SEQ ID NO: 624, respectively, with collectively a total of one or more (e.g., one, two, three, four, five, six, seven, eight, nine, or ten) amino acid positions in SEQ ID NOs: 622-624 substituted with a histidine; and a light chain variable domain comprising a CDR1, a CDR2, and a CDR3 of SEQ ID NO: 625, SEQ ID NO: 626, and SEQ ID NO: 627, respectively, with collectively a total of one or more (e.g., one, two, three, four, five, six, seven, eight, nine, or ten) amino acid positions in SEQ ID NOs: 625-627 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 119 selected from the group consisting of: 32, 50, 52, 53, 57, 58, 59, 60, 62, 64, 65, 97, 98, 99, 100, 102, 104, and 105. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 120, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 120 selected from the group consisting of: 29, 32, 34, 91, 92, 93, 94 and 96. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 119 selected from the group consisting of: 32, 50, 52, 53, 57, 58, 59, 60, 62, 64, 65, 97, 98, 99, 100, 102, 104, and 105, and a light chain variable domain that is at least 90% (e.g., at least 92%, at least 94%, at least 96%, at least 98%, at least 99%, or 100%) identical to SEQ ID NO: 120, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 120 selected from the group consisting of: 29, 32, 34, 91, 92, 93, 94 and 96.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 119, where the heavy chain variable domain includes an alanine at position 35 in SEQ ID NO: 119.

In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 119, where the heavy chain variable domain includes an alanine at position 35 in SEQ ID NO: 119, and a light chain variable domain that is at least 90% (e.g., at least 92%, at least 94%, at least 96%, at least 98%, at least 99%, or 100%) identical to SEQ ID NO: 120, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 120 selected from the group consisting of: 29, 32, 34, 91, 92, 93, 94 and 96.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 119 selected from the group consisting of: 32, 50, 52, 53, 57, 58, 59, 60, 62, 64, 65, 97, 98, 99, 100, 102, 104, and 105, and where the heavy chain variable domain includes an alanine at position 35 in SEQ ID NO: 119.

In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 119 selected from the group consisting of: 32, 50, 52, 53, 57, 58, 59, 60, 62, 64, 65, 97, 98, 99, 100, 102, 104, and 105, and where the heavy chain variable domain includes an alanine at position 35 in SEQ ID NO: 119, and a light chain variable domain that is at least 90% (e.g., at least 92%, at least 94%, at least 96%, at least 98%, at least 99%, or 100%) identical to SEQ ID NO: 120, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 120 selected from the group consisting of 29, 32, 34, 91, 92, 93, 94 and 96.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3 and where the heavy chain variable domain includes an alanine at position 35 of SEQ ID NO: 119.

TABLE 3
Exemplary Combinations of Amino Acid Positions in SEQ
ID NO: 119 that can be substituted with histidine

32 + 50, 32 + 52, 32 + 53, 32 + 57, 32 + 58, 32 + 59, 32 + 60, 32 + 62, 32 + 64, 32 + 65, 32 +
97, 32 + 98, 32 + 99, 32 + 100, 32 + 102, 32 + 104, 32 + 105, 50 + 52, 50 + 53, 50 + 57, 50 +
58, 50 + 59, 50 + 60, 50 + 62, 50 + 64, 50 + 65, 50 + 97, 50 + 98, 50 + 99, 50 + 100, 50 + 102,
50 + 104, 50 + 105, 52 + 53, 52 + 57, 52 + 58, 52 + 59, 52 + 60, 52 + 62, 52 + 64, 52 + 65, 52 +
97, 52 + 98, 52 + 99, 52 + 100, 52 + 102, 52 + 104, 52 + 105, 53 + 57, 53 + 58, 53 + 59, 53 +
60, 53 + 62, 53 + 64, 53 + 65, 53 + 97, 53 + 98, 53 + 99, 53 + 100, 53 + 102, 53 + 104, 53 +
105, 57 + 58, 57 + 59, 57 + 60, 57 + 62, 57 + 64, 57 + 65, 57 + 97, 57 + 98, 57 + 99, 57 + 100,
57 + 102, 57 + 104, 57 + 105, 58 + 59, 58 + 60, 58 + 62, 58 + 64, 58 + 65, 58 + 97, 58 + 98,
58 + 99, 58 + 100, 58 + 102, 58 + 104, 58 + 105, 59 + 60, 59 + 62, 59 + 64, 59 + 65, 59 + 97,
59 + 98, 59 + 99, 59 + 100, 59 + 102, 59 + 104, 59 + 105, 60 + 62, 60 + 64, 60 + 65, 60 + 97,
60 + 98, 60 + 99, 60 + 100, 60 + 102, 60 + 104, 60 + 105, 62 + 64, 62 + 65, 62 + 97, 62 + 98,
62 + 99, 62 + 100, 62 + 102, 62 + 104, 62 + 105, 64 + 65, 64 + 97, 64 + 98, 64 + 99, 64 + 100,
64 + 102, 64 + 104, 64 + 105, 65 + 97, 65 + 98, 65 + 99, 65 + 100, 65 + 102, 65 + 104, 65 +
105, 97 + 98, 97 + 99, 97 + 100, 97 + 102, 97 + 104, 97 + 105, 98 + 99, 98 + 100, 98 + 102,
98 + 104, 98 + 105, 99 + 100, 99 + 102, 99 + 104, 99 + 105, 100 + 102, 100 + 104, 100 + 105,
102 + 104, 102 + 105, 104 + 105, 32 + 50 + 52, 32 + 50 + 53, 32 + 50 + 57, 32 + 50 + 58, 32 +
50 + 59, 32 + 50 + 60, 32 + 50 + 62, 32 + 50 + 64, 32 + 50 + 65, 32 + 50 + 97, 32 + 50 + 98,
32 + 50 + 99, 32 + 50 + 100, 32 + 50 + 102, 32 + 50 + 104, 32 + 50 + 105, 32 + 52 + 53, 32 +
52 + 57, 32 + 52 + 58, 32 + 52 + 59, 32 + 52 + 60, 32 + 52 + 62, 32 + 52 + 64, 32 + 52 + 65,
32 + 52 + 97, 32 + 52 + 98, 32 + 52 + 99, 32 + 52 + 100, 32 + 52 + 102, 32 + 52 + 104, 32 +
52 + 105, 32 + 53 + 57, 32 + 53 + 58, 32 + 53 + 59, 32 + 53 + 60, 32 + 53 + 62, 32 + 53 + 64,
32 + 53 + 65, 32 + 53 + 97, 32 + 53 + 98, 32 + 53 + 99, 32 + 53 + 100, 32 + 53 + 102, 32 + 53 +
104, 32 + 53 + 105, 32 + 57 + 58, 32 + 57 + 59, 32 + 57 + 60, 32 + 57 + 62, 32 + 57 + 64,
32 + 57 + 65, 32 + 57 + 97, 32 + 57 + 98, 32 + 57 + 99, 32 + 57 + 100, 32 + 57 + 102, 32 + 57 +
104, 32 + 57 + 105, 32 + 58 + 59, 32 + 58 + 60, 32 + 58 + 62, 32 + 58 + 64, 32 + 58 + 65,
32 + 58 + 97, 32 + 58 + 98, 32 + 58 + 99, 32 + 58 + 100, 32 + 58 + 102, 32 + 58 + 104, 32 +
58 + 105, 32 + 59 + 60, 32 + 59 + 62, 32 + 59 + 64, 32 + 59 + 65, 32 + 59 + 97, 32 + 59 + 98,
32 + 59 + 99, 32 + 59 + 100, 32 + 59 + 102, 32 + 59 + 104, 32 + 59 + 105, 32 + 60 + 62, 32 +
60 + 64, 32 + 60 + 65, 32 + 60 + 97, 32 + 60 + 98, 32 + 60 + 99, 32 + 60 + 100, 32 + 60 +
102, 32 + 60 + 104, 32 + 60 + 105, 32 + 62 + 64, 32 + 62 + 65, 32 + 62 + 97, 32 + 62 + 98, 32 +
62 + 99, 32 + 62 + 100, 32 + 62 + 102, 32 + 62 + 104, 32 + 62 + 105, 32 + 64 + 65, 32 + 64 +
97, 32 + 64 + 98, 32 + 64 + 99, 32 + 64 + 100, 32 + 64 + 102, 32 + 64 + 104, 32 + 64 + 105,
32 + 65 + 97, 32 + 65 + 98, 32 + 65 + 99, 32 + 65 + 100, 32 + 65 + 102, 32 + 65 + 104, 32 +
65 + 105, 32 + 97 + 98, 32 + 97 + 99, 32 + 97 + 100, 32 + 97 + 102, 32 + 97 + 104, 32 + 97 +
105, 32 + 98 + 99, 32 + 98 + 100, 32 + 98 + 102, 32 + 98 + 104, 32 + 98 + 105, 32 + 99 + 100,
32 + 99 + 102, 32 + 99 + 104, 32 + 99 + 105, 32 + 100 + 102, 32 + 100 + 104, 32 + 100 + 105,
32 + 102 + 104, 32 + 102 + 105, 32 + 104 + 105, 50 + 52 + 53, 50 + 52 + 57, 50 + 52 + 58, 50 +
52 + 59, 50 + 52 + 60, 50 + 52 + 62, 50 + 52 + 64, 50 + 52 + 65, 50 + 52 + 97, 50 + 52 + 98,
50 + 52 + 99, 50 + 52 + 100, 50 + 52 + 102, 50 + 52 + 104, 50 + 52 + 105, 50 + 53 + 57, 50 +
53 + 58, 50 + 53 + 59, 50 + 53 + 60, 50 + 53 + 62, 50 + 53 + 64, 50 + 53 + 65, 50 + 53 + 97,
50 + 53 + 98, 50 + 53 + 99, 50 + 53 + 100, 50 + 53 + 102, 50 + 53 + 104, 50 + 53 + 105, 50 +
57 + 58, 50 + 57 + 59, 50 + 57 + 60, 50 + 57 + 62, 50 + 57 + 64, 50 + 57 + 65, 50 + 57 + 97,
50 + 57 + 98, 50 + 57 + 99, 50 + 57 + 100, 50 + 57 + 102, 50 + 57 + 104, 50 + 57 + 105, 50 +
58 + 59, 50 + 58 + 60, 50 + 58 + 62, 50 + 58 + 64, 50 + 58 + 65, 50 + 58 + 97, 50 + 58 + 98,
50 + 58 + 99, 50 + 58 + 100, 50 + 58 + 102, 50 + 58 + 104, 50 + 58 + 105, 50 + 59 + 60, 50 +
59 + 62, 50 + 59 + 64, 50 + 59 + 65, 50 + 59 + 97, 50 + 59 + 98, 50 + 59 + 99, 50 + 59 + 100,
50 + 59 + 102, 50 + 59 + 104, 50 + 59 + 105, 50 + 60 + 62, 50 + 60 + 64, 50 + 60 + 65, 50 +
60 + 97, 50 + 60 + 98, 50 + 60 + 99, 50 + 60 + 100, 50 + 60 + 102, 50 + 60 + 104, 50 + 60 +
105, 50 + 62 + 64, 50 + 62 + 65, 50 + 62 + 97, 50 + 62 + 98, 50 + 62 + 99, 50 + 62 + 100, 50 +
62 + 102, 50 + 62 + 104, 50 + 62 + 105, 50 + 64 + 65, 50 + 64 + 97, 50 + 64 + 98, 50 + 64 +
99, 50 + 64 + 100, 50 + 64 + 102, 50 + 64 + 104, 50 + 64 + 105, 50 + 65 + 97, 50 + 65 + 98,
50 + 65 + 99, 50 + 65 + 100, 50 + 65 + 102, 50 + 65 + 104, 50 + 65 + 105, 50 + 97 + 98, 50 +
97 + 99, 50 + 97 + 100, 50 + 97 + 102, 50 + 97 + 104, 50 + 97 + 105, 50 + 98 + 99, 50 + 98 +
100, 50 + 98 + 102, 50 + 98 + 104, 50 + 98 + 105, 50 + 99 + 100, 50 + 99 + 102, 50 + 99 +
104, 50 + 99 + 105, 50 + 100 + 102, 50 + 100 + 104, 50 + 100 + 105, 50 + 102 + 104, 50 +
102 + 105, 50 + 104 + 105, 52 + 53 + 57, 52 + 53 + 58, 52 + 53 + 59, 52 + 53 + 60, 52 + 53 +
62, 52 + 53 + 64, 52 + 53 + 65, 52 + 53 + 97, 52 + 53 + 98, 52 + 53 + 99, 52 + 53 + 100, 52 +
53 + 102, 52 + 53 + 104, 52 + 53 + 105, 52 + 57 + 58, 52 + 57 + 59, 52 + 57 + 60, 52 + 57 +
62, 52 + 57 + 64, 52 + 57 + 65, 52 + 57 + 97, 52 + 57 + 98, 52 + 57 + 99, 52 + 57 + 100, 52 +
57 + 102, 52 + 57 + 104, 52 + 57 + 105, 52 + 58 + 59, 52 + 58 + 60, 52 + 58 + 62, 52 + 58 +
64, 52 + 58 + 65, 52 + 58 + 97, 52 + 58 + 98, 52 + 58 + 99, 52 + 58 + 100, 52 + 58 + 102, 52 +
58 + 104, 52 + 58 + 105, 52 + 59 + 60, 52 + 59 + 62, 52 + 59 + 64, 52 + 59 + 65, 52 + 59 +
97, 52 + 59 + 98, 52 + 59 + 99, 52 + 59 + 100, 52 + 59 + 102, 52 + 59 + 104, 52 + 59 + 105,
52 + 60 + 62, 52 + 60 + 64, 52 + 60 + 65, 52 + 60 + 97, 52 + 60 + 98, 52 + 60 + 99, 52 + 60 +
100, 52 + 60 + 102, 52 + 60 + 104, 52 + 60 + 105, 52 + 62 + 64, 52 + 62 + 65, 52 + 62 + 97,
52 + 62 + 98, 52 + 62 + 99, 52 + 62 + 100, 52 + 62 + 102, 52 + 62 + 104, 52 + 62 + 105, 52 +
64 + 65, 52 + 64 + 97, 52 + 64 + 98, 52 + 64 + 99, 52 + 64 + 100, 52 + 64 + 102, 52 + 64 +
104, 52 + 64 + 105, 52 + 65 + 97, 52 + 65 + 98, 52 + 65 + 99, 52 + 65 + 100, 52 + 65 + 102,
52 + 65 + 104, 52 + 65 + 105, 52 + 97 + 98, 52 + 97 + 99, 52 + 97 + 100, 52 + 97 + 102, 52 +
97 + 104, 52 + 97 + 105, 52 + 98 + 99, 52 + 98 + 100, 52 + 98 + 102, 52 + 98 + 104, 52 + 98 +
105, 52 + 99 + 100, 52 + 99 + 102, 52 + 99 + 104, 52 + 99 + 105, 52 + 100 + 102, 52 + 100 +
104, 52 + 100 + 105, 52 + 102 + 104, 52 + 102 + 105, 52 + 104 + 105, 53 + 57 + 58, 53 + 57 +
59, 53 + 57 + 60, 53 + 57 + 62, 53 + 57 + 64, 53 + 57 + 65, 53 + 57 + 97, 53 + 57 + 98, 53 +
57 + 99, 53 + 57 + 100, 53 + 57 + 102, 53 + 57 + 104, 53 + 57 + 105, 53 + 58 + 59, 53 + 58 +
60, 53 + 58 + 62, 53 + 58 + 64, 53 + 58 + 65, 53 + 58 + 97, 53 + 58 + 98, 53 + 58 + 99, 53 +
58 + 100, 53 + 58 + 102, 53 + 58 + 104, 53 + 58 + 105, 53 + 59 + 60, 53 + 59 + 62, 53 + 59 +
64, 53 + 59 + 65, 53 + 59 + 97, 53 + 59 + 98, 53 + 59 + 99, 53 + 59 + 100, 53 + 59 + 102, 53 +
59 + 104, 53 + 59 + 105, 53 + 60 + 62, 53 + 60 + 64, 53 + 60 + 65, 53 + 60 + 97, 53 + 60 +
98, 53 + 60 + 99, 53 + 60 + 100, 53 + 60 + 102, 53 + 60 + 104, 53 + 60 + 105, 53 + 62 + 64,
53 + 62 + 65, 53 + 62 + 97, 53 + 62 + 98, 53 + 62 + 99, 53 + 62 + 100, 53 + 62 + 102, 53 + 62 +
104, 53 + 62 + 105, 53 + 64 + 65, 53 + 64 + 97, 53 + 64 + 98, 53 + 64 + 99, 53 + 64 + 100,
53 + 64 + 102, 53 + 64 + 104, 53 + 64 + 105, 53 + 65 + 97, 53 + 65 + 98, 53 + 65 + 99, 53 +
65 + 100, 53 + 65 + 102, 53 + 65 + 104, 53 + 65 + 105, 53 + 97 + 98, 53 + 97 + 99, 53 + 97 +
100, 53 + 97 + 102, 53 + 97 + 104, 53 + 97 + 105, 53 + 98 + 99, 53 + 98 + 100, 53 + 98 + 102,
53 + 98 + 104, 53 + 98 + 105, 53 + 99 + 100, 53 + 99 + 102, 53 + 99 + 104, 53 + 99 + 105, 53 +
100 + 102, 53 + 100 + 104, 53 + 100 + 105, 53 + 102 + 104, 53 + 102 + 105, 53 + 104 +
105, 57 + 58 + 59, 57 + 58 + 60, 57 + 58 + 62, 57 + 58 + 64, 57 + 58 + 65, 57 + 58 + 97, 57 +
58 + 98, 57 + 58 + 99, 57 + 58 + 100, 57 + 58 + 102, 57 + 58 + 104, 57 + 58 + 105, 57 + 59 +
60, 57 + 59 + 62, 57 + 59 + 64, 57 + 59 + 65, 57 + 59 + 97, 57 + 59 + 98, 57 + 59 + 99, 57 +
59 + 100, 57 + 59 + 102, 57 + 59 + 104, 57 + 59 + 105, 57 + 60 + 62, 57 + 60 + 64, 57 + 60 +
65, 57 + 60 + 97, 57 + 60 + 98, 57 + 60 + 99, 57 + 60 + 100, 57 + 60 + 102, 57 + 60 + 104, 57 +
60 + 105, 57 + 62 + 64, 57 + 62 + 65, 57 + 62 + 97, 57 + 62 + 98, 57 + 62 + 99, 57 + 62 +
100, 57 + 62 + 102, 57 + 62 + 104, 57 + 62 + 105, 57 + 64 + 65, 57 + 64 + 97, 57 + 64 + 98,
57 + 64 + 99, 57 + 64 + 100, 57 + 64 + 102, 57 + 64 + 104, 57 + 64 + 105, 57 + 65 + 97, 57 +
65 + 98, 57 + 65 + 99, 57 + 65 + 100, 57 + 65 + 102, 57 + 65 + 104, 57 + 65 + 105, 57 + 97 +
98, 57 + 97 + 99, 57 + 97 + 100, 57 + 97 + 102, 57 + 97 + 104, 57 + 97 + 105, 57 + 98 + 99,
57 + 98 + 100, 57 + 98 + 102, 57 + 98 + 104, 57 + 98 + 105, 57 + 99 + 100, 57 + 99 + 102, 57 +
99 + 104, 57 + 99 + 105, 57 + 100 + 102, 57 + 100 + 104, 57 + 100 + 105, 57 + 102 + 104,
57 + 102 + 105, 57 + 104 + 105, 58 + 59 + 60, 58 + 59 + 62, 58 + 59 + 64, 58 + 59 + 65, 58 +
59 + 97, 58 + 59 + 98, 58 + 59 + 99, 58 + 59 + 100, 58 + 59 + 102, 58 + 59 + 104, 58 + 59 +
105, 58 + 60 + 62, 58 + 60 + 64, 58 + 60 + 65, 58 + 60 + 97, 58 + 60 + 98, 58 + 60 + 99, 58 +
60 + 100, 58 + 60 + 102, 58 + 60 + 104, 58 + 60 + 105, 58 + 62 + 64, 58 + 62 + 65, 58 + 62 +
97, 58 + 62 + 98, 58 + 62 + 99, 58 + 62 + 100, 58 + 62 + 102, 58 + 62 + 104, 58 + 62 + 105,
58 + 64 + 65, 58 + 64 + 97, 58 + 64 + 98, 58 + 64 + 99, 58 + 64 + 100, 58 + 64 + 102, 58 + 64 +
104, 58 + 64 + 105, 58 + 65 + 97, 58 + 65 + 98, 58 + 65 + 99, 58 + 65 + 100, 58 + 65 + 102,
58 + 65 + 104, 58 + 65 + 105, 58 + 97 + 98, 58 + 97 + 99, 58 + 97 + 100, 58 + 97 + 102, 58 +
97 + 104, 58 + 97 + 105, 58 + 98 + 99, 58 + 98 + 100, 58 + 98 + 102, 58 + 98 + 104, 58 + 98 +
105, 58 + 99 + 100, 58 + 99 + 102, 58 + 99 + 104, 58 + 99 + 105, 58 + 100 + 102, 58 + 100 +
104, 58 + 100 + 105, 58 + 102 + 104, 58 + 102 + 105, 58 + 104 + 105, 59 + 60 + 62, 59 + 60 +
64, 59 + 60 + 65, 59 + 60 + 97, 59 + 60 + 98, 59 + 60 + 99, 59 + 60 + 100, 59 + 60 + 102,
59 + 60 + 104, 59 + 60 + 105, 59 + 62 + 64, 59 + 62 + 65, 59 + 62 + 97, 59 + 62 + 98, 59 + 62 +
99, 59 + 62 + 100, 59 + 62 + 102, 59 + 62 + 104, 59 + 62 + 105, 59 + 64 + 65, 59 + 64 + 97,
59 + 64 + 98, 59 + 64 + 99, 59 + 64 + 100, 59 + 64 + 102, 59 + 64 + 104, 59 + 64 + 105, 59 +
65 + 97, 59 + 65 + 98, 59 + 65 + 99, 59 + 65 + 100, 59 + 65 + 102, 59 + 65 + 104, 59 + 65 +
105, 59 + 97 + 98, 59 + 97 + 99, 59 + 97 + 100, 59 + 97 + 102, 59 + 97 + 104, 59 + 97 + 105,
59 + 98 + 99, 59 + 98 + 100, 59 + 98 + 102, 59 + 98 + 104, 59 + 98 + 105, 59 + 99 + 100, 59 +
99 + 102, 59 + 99 + 104, 59 + 99 + 105, 59 + 100 + 102, 59 + 100 + 104, 59 + 100 + 105, 59 +
102 + 104, 59 + 102 + 105, 59 + 104 + 105, 60 + 62 + 64, 60 + 62 + 65, 60 + 62 + 97, 60 +
62 + 98, 60 + 62 + 99, 60 + 62 + 100, 60 + 62 + 102, 60 + 62 + 104, 60 + 62 + 105, 60 + 64 +
65, 60 + 64 + 97, 60 + 64 + 98, 60 + 64 + 99, 60 + 64 + 100, 60 + 64 + 102, 60 + 64 + 104, 60 +
64 + 105, 60 + 65 + 97, 60 + 65 + 98, 60 + 65 + 99, 60 + 65 + 100, 60 + 65 + 102, 60 + 65 +
104, 60 + 65 + 105, 60 + 97 + 98, 60 + 97 + 99, 60 + 97 + 100, 60 + 97 + 102, 60 + 97 + 104,
60 + 97 + 105, 60 + 98 + 99, 60 + 98 + 100, 60 + 98 + 102, 60 + 98 + 104, 60 + 98 + 105, 60 +
99 + 100, 60 + 99 + 102, 60 + 99 + 104, 60 + 99 + 105, 60 + 100 + 102, 60 + 100 + 104, 60 +
100 + 105, 60 + 102 + 104, 60 + 102 + 105, 60 + 104 + 105, 62 + 64 + 65, 62 + 64 + 97, 62 +
64 + 98, 62 + 64 + 99, 62 + 64 + 100, 62 + 64 + 102, 62 + 64 + 104, 62 + 64 + 105, 62 + 65 +
97, 62 + 65 + 98, 62 + 65 + 99, 62 + 65 + 100, 62 + 65 + 102, 62 + 65 + 104, 62 + 65 + 105,
62 + 97 + 98, 62 + 97 + 99, 62 + 97 + 100, 62 + 97 + 102, 62 + 97 + 104, 62 + 97 + 105, 62 +
98 + 99, 62 + 98 + 100, 62 + 98 + 102, 62 + 98 + 104, 62 + 98 + 105, 62 + 99 + 100, 62 + 99 +
102, 62 + 99 + 104, 62 + 99 + 105, 62 + 100 + 102, 62 + 100 + 104, 62 + 100 + 105, 62 +
102 + 104, 62 + 102 + 105, 62 + 104 + 105, 64 + 65 + 97, 64 + 65 + 98, 64 + 65 + 99, 64 + 65 +
100, 64 + 65 + 102, 64 + 65 + 104, 64 + 65 + 105, 64 + 97 + 98, 64 + 97 + 99, 64 + 97 +
100, 64 + 97 + 102, 64 + 97 + 104, 64 + 97 + 105, 64 + 98 + 99, 64 + 98 + 100, 64 + 98 + 102,
64 + 98 + 104, 64 + 98 + 105, 64 + 99 + 100, 64 + 99 + 102, 64 + 99 + 104, 64 + 99 + 105, 64 +
100 + 102, 64 + 100 + 104, 64 + 100 + 105, 64 + 102 + 104, 64 + 102 + 105, 64 + 104 +
105, 65 + 97 + 98, 65 + 97 + 99, 65 + 97 + 100, 65 + 97 + 102, 65 + 97 + 104, 65 + 97 + 105,
65 + 98 + 99, 65 + 98 + 100, 65 + 98 + 102, 65 + 98 + 104, 65 + 98 + 105, 65 + 99 + 100, 65 +
99 + 102, 65 + 99 + 104, 65 + 99 + 105, 65 + 100 + 102, 65 + 100 + 104, 65 + 100 + 105, 65 +
102 + 104, 65 + 102 + 105, 65 + 104 + 105, 97 + 98 + 99, 97 + 98 + 100, 97 + 98 + 102, 97 +
98 + 104, 97 + 98 + 105, 97 + 99 + 100, 97 + 99 + 102, 97 + 99 + 104, 97 + 99 + 105, 97 +
100 + 102, 97 + 100 + 104, 97 + 100 + 105, 97 + 102 + 104, 97 + 102 + 105, 97 + 104 + 105,
98 + 99 + 100, 98 + 99 + 102, 98 + 99 + 104, 98 + 99 + 105, 98 + 100 + 102, 98 + 100 + 104,
98 + 100 + 105, 98 + 102 + 104, 98 + 102 + 105, 98 + 104 + 105, 99 + 100 + 102, 99 + 100 +
104, 99 + 100 + 105, 99 + 102 + 104, 99 + 102 + 105, 99 + 104 + 105, 100 + 102 + 104, 100 +
102 + 105, 100 + 104 + 105, 102 + 104 + 105,

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 120, where the light chain variable domain includes a histidine at any of the specific combinations of one or more amino acid positions in SEQ ID NO: 120 listed in Table 4.

TABLE 4
Exemplary Combinations of Amino Acid Positions in SEQ
ID NO: 120 that can be substituted with histidine

29 + 32, 29 + 34, 29 + 91, 29 + 92, 29 + 93, 29 + 94, 29 + 96, 32 + 34, 32 + 91, 32 + 92,
32 + 93, 32 + 94, 32 + 96, 34 + 91, 34 + 92, 34 + 93, 34 + 94, 34 + 96, 91 + 92, 91 + 93,
91 + 94, 91 + 96, 92 + 93, 92 + 94, 92 + 96, 93 + 94, 93 + 96, 94 + 96, 29 + 32 + 34,
29 + 32 + 91, 29 + 32 + 92, 29 + 32 + 93, 29 + 32 + 94, 29 + 32 + 96, 29 + 34 + 91, 29 + 34 +
92, 29 + 34 + 93, 29 + 34 + 94, 29 + 34 + 96, 29 + 91 + 92, 29 + 91 + 93, 29 + 91 + 94, 29 +
91 + 96, 29 + 92 + 93, 29 + 92 + 94, 29 + 92 + 96, 29 + 93 + 94, 29 + 93 + 96, 29 + 94 + 96,
32 + 34 + 91, 32 + 34 + 92, 32 + 34 + 93, 32 + 34 + 94, 32 + 34 + 96, 32 + 91 + 92, 32 + 91 +
93, 32 + 91 + 94, 32 + 91 + 96, 32 + 92 + 93, 32 + 92 + 94, 32 + 92 + 96, 32 + 93 + 94, 32 +
93 + 96, 32 + 94 + 96, 34 + 91 + 92, 34 + 91 + 93, 34 + 91 + 94, 34 + 91 + 96, 34 + 92 + 93,
34 + 92 + 94, 34 + 92 + 96, 34 + 93 + 94, 34 + 93 + 96, 34 + 94 + 96, 91 + 92 + 93, 91 + 92 +
94, 91 + 92 + 96, 91 + 93 + 94, 91 + 93 + 96, 91 + 94 + 96, 92 + 93 + 94, 92 + 93 + 96, 92 +
94 + 96, 93 + 94 + 96

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3; and a light chain variable domain that that is at least 90% (e.g., at least 92%, at least 94%, at least 96%, at least 98%, at least 99%, or 100%) identical to SEQ ID NO: 120, where the light chain variable domain includes a histidine at any of the specific combinations of one or more amino acid positions in SEQ ID NO: 120 listed in Table 4.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3, and where the heavy chain variable domain includes an alanine at position 35 in SEQ ID NO: 119; and a light chain variable domain that that is at least 90% (e.g., at least 92%, at least 94%, at least 96%, at least 98%, at least 99%, or 100%) identical to SEQ ID NO: 120, where the light chain variable domain includes a histidine at any of the specific combinations of one or more amino acid positions in SEQ ID NO: 120 listed in Table 4.

In some examples of any of the ABPCs described herein, the first antigen-binding domain comprises a light chain variable domain that is at least 90% identical to SEQ ID NO: 120, wherein the light chain variable domain includes a histidine at position 29 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain comprising SEQ ID NO: 120, and a heavy chain variable domain that is at least 90% identical (e.g., at least 92%, at least 94%, at least 96%, at least 98%, at least 99%, or 100%) identical to SEQ ID NO: 119, where the heavy chain variable domain includes a histidine at any of the specific combinations of one or more (e.g., two, three, four, five, six, seven, eight, nine, or ten) amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 32 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 34 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 91 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 92 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 93 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 94 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain comprises a light chain variable domain that is at least 90% identical to SEQ ID NO: 120, wherein the light chain variable domain includes a histidine at position 96 in SEQ ID NO: 120; and a heavy chain variable domain that is at least 90% (e.g., at least 92%, at least 94%, at least 96%, at least 98%, at least 99%, or 100%) identical to SEQ ID NO: 119, where the heavy chain variable domain includes a histidine at any of the specific combinations of one or more amino acid positions in SEQ ID NO: 119 listed in Table 3.

In some examples of any of the ABPCs described herein, the first antigen-binding domain includes a heavy chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.4 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.4 comprises SEQ ID NO: 119. In some examples of any of the ABPCs described herein, the light chain variable domain of SC16.4 comprises SEQ ID NO: 120.

In some examples of any of the ABPCs described herein, the first antigen-binding domain comprises a heavy chain variable domain comprising a CDR1, a CDR2, and a CDR3 of SEQ ID NO: 622, SEQ ID NO: 623, and SEQ ID NO: 624, respectively, with collectively a total of one or more (e.g., one, two, three, four, five, six, seven, eight, nine, or ten) histidine(s) in SEQ ID NOs: 622-624 substituted with an alanine. In some examples of any of the ABPCs described herein, the first antigen-binding domain comprises a light chain variable domain comprising a CDR1, a CDR2, and a CDR3 of SEQ ID NO: 625, SEQ ID NO: 626, and SEQ ID NO: 627, respectively, with collectively a total of one or more (e.g., one, two, three, four, five, six, seven, eight, nine, or ten) histidine(s) in SEQ ID NOs: 625-628 substituted with an alanine. In some examples of any of the ABPCs described herein, the first antigen-binding domain includes: a heavy chain variable domain comprising a CDR1, a CDR2, and a CDR3 of SEQ ID NO: 622, SEQ ID NO: 623, and SEQ ID NO: 624, respectively, with collectively a total of one or more (e.g., one, two, three, four, five, six, seven, eight, nine, or ten) histidine(s) in SEQ ID NOs: 622-624 substituted with an alanine; and a light chain variable domain comprising a CDR1, a CDR2, and a CDR3 of SEQ ID NO: 625, SEQ ID NO: 626, and SEQ ID NO: 627, respectively, with collectively a total of one or more (e.g., one, two, three, four, five, six, seven, eight, nine, or ten) histidine(s) in SEQ ID NOs: 625-627 substituted with an alanine.

In some examples of any of the ABPCs described herein, the first antigen-binding domain comprises a heavy chain variable domain of SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain of SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of the ABPCs described herein, the first antigen-binding domain includes a light chain variable domain comprising SEQ ID NO: 120, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 158, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 159, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 160, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 161, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 162, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 163, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 164, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 165, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 166, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 167, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 168, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 169, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 170, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 171, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 172, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 173, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 174, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 175, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 176, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 177, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 178, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 179, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 180, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 181, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 182, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 183, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 184, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a heavy chain variable domain comprising SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, or SEQ ID NO: 225.

In some examples of any of ABPCs described herein, the first-antigen binding domain includes a heavy chain variable domain comprising SEQ ID NO: 185, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 186, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 187, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 188, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 189, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 190, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 191, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 192, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 193, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 194, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 195, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 196, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 197, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 198, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 199, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 200, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 201, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 202, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 203, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 204, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 205, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 206, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 207, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 208, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 209, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 210, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 211, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 212, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 213, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 214, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 215, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 216, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 217, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 218, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 219, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 220, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 221, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 222, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 223, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 224, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of ABPCs described herein, the first-antigen binding domain includes a heavy chain variable domain comprising SEQ ID NO: 225, and a light chain variable domain comprising SEQ ID NO: 120, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, or SEQ ID NO: 184.

In some examples of any of the ABPCs described herein, the first antigen-binding domain includes a light chain variable domain comprising SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, and SEQ ID NO: 235.

In some examples of any of the ABPCs described herein, the first antigen-binding domain includes a light chain variable domain comprising SEQ IS NO: 226, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 227, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 228, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 229, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 230, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 231, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 232, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 233, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 234, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ IS NO: 235, and a heavy chain variable domain comprising SEQ ID NO: 119, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, or SEQ ID NO: 157.

In some examples of any of the ABPCs described herein, the first antigen-binding domain includes a light chain variable domain comprising SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, and SEQ ID NO: 235 and heavy chain variable domain comprising SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, or SEQ ID NO: 225.

In some examples of any of the ABPCs described herein, the first antigen-binding domain includes a light chain variable domain comprising SEQ ID NO: 163, SEQ ID NO: 166, and SEQ ID NO: 179. In some examples of any of the ABPCs described herein, the first antigen binding domain includes a heavy chain variable domain comprising SEQ ID NO: 127, SEQ ID NO: 138, SEQ ID NO: 141, SEQ ID NO: 143, SEQ ID NO: 150, SEQ ID NO: 151, or SEQ ID NO: 156. In some examples of any of the ABPCs described herein, the first antigen-binding domain includes a light chain variable domain comprising SEQ ID NO: 163, SEQ ID NO: 166, and SEQ ID NO: 179, and a heavy chain variable domain comprising SEQ ID NO: 127, SEQ ID NO: 138, SEQ ID NO: 141, SEQ ID NO: 143, SEQ ID NO: 150, SEQ ID NO: 151, or SEQ ID NO: 156.

In some examples of any of the ABPCs described herein, the first antigen-binding domain includes a light chain variable domain comprising SEQ ID NO: 163, and a heavy chain variable domain comprising SEQ ID NO: 127, SEQ ID NO: 138, SEQ ID NO: 141, SEQ ID NO: 143, SEQ ID NO: 150, SEQ ID NO: 151, SEQ ID NO: 156.

In some examples of any of the ABPCs described herein, the first antigen-binding domain includes a light chain variable domain comprising SEQ ID NO: 166, and a heavy chain variable domain comprising SEQ ID NO: 127, SEQ ID NO: 138, SEQ ID NO: 141, SEQ ID NO: 143, SEQ ID NO: 150, SEQ ID NO: 151, SEQ ID NO: 156.

In some examples of any of the ABPCs described herein, the first antigen-binding domain includes a light chain variable domain comprising SEQ ID NO: 179, and a heavy chain variable domain comprising SEQ ID NO: 127, SEQ ID NO: 138, SEQ ID NO: 141, SEQ ID NO: 143, SEQ ID NO: 150, SEQ ID NO: 151, SEQ ID NO: 156.

In some examples of any of the ABPCs described herein, the first antigen-binding domain includes a heavy chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.13 comprises SEQ ID NO: 236. In some examples of any of the ABPCs described herein, the light chain variable domain of SC16.13 comprises SEQ ID NO: 237.

In some examples of any of the ABPCs described herein, the first antigen-binding domain comprises a heavy chain variable domain comprising a CDR1, a CDR2, and a CDR3 of SEQ ID NO: 628, SEQ ID NO: 629, and SEQ ID NO: 630, respectively, with collectively a total of one or more (e.g., one, two, three, four, five, six, seven, eight, nine, or ten) amino acid positions in SEQ ID NOs: 628-630 substituted with a histidine. In some examples of any of the ABPCs described herein, the first antigen-binding domain comprises a light chain variable domain comprising a CDR1, a CDR2, and a CDR3 of SEQ ID NO: 631, SEQ ID NO: 632, and SEQ ID NO: 633, respectively, with collectively a total of one or more (e.g., one, two, three, four, five, six, seven, eight, nine, or ten) amino acid positions in SEQ ID NOs: 631-633 substituted with a histidine. In some examples of any of the ABPCs described herein, the first antigen-binding domain includes: a heavy chain variable domain comprising a CDR1, a CDR2, and a CDR3 of SEQ ID NO: 628, SEQ ID NO: 629, and SEQ ID NO: 630, respectively, with collectively a total of one or more (e.g., one, two, three, four, five, six, seven, eight, nine, or ten) amino acid positions in SEQ ID NOs: 628-630 substituted with a histidine; and a light chain variable domain comprising a CDR1, a CDR2, and a CDR3 of SEQ ID NO: 631, SEQ ID NO: 632, and SEQ ID NO: 633, respectively, with collectively a total of one or more (e.g., one, two, three, four, five, six, seven, eight, nine, or ten) amino acid positions in SEQ ID NOs: 631-633 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 236 selected from the group consisting of: 27, 29, 31, 33, 34, 35, 36, 37, 54, 55, 56, 58, 60, 98, 100, 102, 106, 108, and 110. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 237, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 237 selected from the group consisting of: 25, 29, 32, 33, 49, 50, 90, 91, 93, and 95. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 236 selected from the group consisting of: 27, 29, 31, 33, 34, 35, 36, 37, 54, 55, 56, 58, 60, 98, 100, 102, 106, 108, and 110, and a light chain variable domain that is at least 90% (e.g., at least 92%, at least 94%, at least 96%, at least 98%, at least 99%, or 100%) identical to SEQ ID NO: 237, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 237 selected from the group consisting of: 25, 29, 32, 33, 49, 50, 90, 91, 93, and 95.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 236, where the heavy chain variable domain includes an alanine at position 52 in SEQ ID NO: 236.

In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 236, where the heavy chain variable domain includes an alanine at position 52 in SEQ ID NO: 236, and a light chain variable domain that is at least 90% (e.g., at least 92%, at least 94%, at least 96%, at least 98%, at least 99%, or 100%) identical to SEQ ID NO: 237, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 237 selected from the group consisting of: 25, 29, 32, 33, 49, 50, 90, 91, 93, and 95.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 236 selected from the group consisting of: 27, 29, 31, 33, 34, 35, 36, 37, 54, 55, 56, 58, 60, 98, 100, 102, 106, 108, and 110, and where the heavy chain variable domain includes an alanine at position 52 in SEQ ID NO: 236. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 236 selected from the group consisting of: 27, 29, 31, 33, 34, 35, 36, 37, 54, 55, 56, 58, 60, 98, 100, 102, 106, 108, and 110, and where the heavy chain variable domain includes an alanine at position 52 in SEQ ID NO: 236, and a light chain variable domain that is at least 90% (e.g., at least 92%, at least 94%, at least 96%, at least 980, at least 990, or 100) identical to SEQ ID NO: 237, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 237 selected from the group consisting of: 25, 29, 32, 33, 49, 50, 90, 91, 93, and 95.

In some examples of any of the ABPCs described herein, a heavy chain variable domain includes a heavy chain variable domain that is at least 90% (e.g., at least 92%, at least 94% at least 96%, at least 98%, at least 99% or 100%) identical to SEQ ID NO: 237, where the heavy chain variable domain includes a histidine at any of the specific combinations of one or more amino acid positions in SEQ ID NO: 237 listed in Table 5 and where the heavy chain variable domain includes an alanine at position 52 of SEQ ID NO: 237.

TABLE 5
Exemplary Combinations of Amino Acid Positions in SEQ
ID NO: 236 that can be substituted with histidine

27 + 29, 27 + 31, 27 + 33, 27 + 34, 27 + 35, 27 + 36, 27 + 37, 27 + 54, 27 + 55, 27 + 56, 27 +
58, 27 + 60, 27 + 98, 27 + 100, 27 + 102, 27 + 106, 27 + 108, 27 + 110, 29 + 31,29 + 33,29 +
34, 29 + 35, 29 + 36, 29 + 37, 29 + 54, 29 + 55, 29 + 56, 29 + 58, 29 + 60, 29 + 98, 29 + 100,
29 + 102, 29 + 106, 29 + 108, 29 + 110, 31 + 33, 31 + 34, 31 + 35, 31 + 36, 31 + 37, 31 + 54,
31 + 55, 31 + 56, 31 + 58, 31 + 60, 31 + 98, 31 + 100, 31 + 102, 31 + 106, 31 + 108, 31 + 110,
33 + 34, 33 + 35, 33 + 36, 33 + 37, 33 + 54, 33 + 55, 33 + 56, 33 + 58, 33 + 60, 33 + 98, 33 +
100, 33 + 102, 33 + 106, 33 + 108, 33 + 110, 34 + 35, 34 + 36, 34 + 37, 34 + 54, 34 + 55, 34 +
56, 34 + 58, 34 + 60, 34 + 98, 34 + 100, 34 + 102, 34 + 106, 34 + 108, 34 + 110, 35 + 36, 35 +
37, 35 + 54, 35 + 55, 35 + 56, 35 + 58, 35 + 60, 35 + 98, 35 + 100, 35 + 102, 35 + 106, 35 +
108, 35 + 110, 36 + 37, 36 + 54, 36 + 55, 36 + 56, 36 + 58, 36 + 60, 36 + 98, 36 + 100, 36 +
102, 36 + 106, 36 + 108, 36 + 110, 37 + 54, 37 + 55, 37 + 56, 37 + 58, 37 + 60, 37 + 98, 37 +
100, 37 + 102, 37 + 106, 37 + 108, 37 + 110, 54 + 55, 54 + 56, 54 + 58, 54 + 60, 54 + 98, 54 +
100, 54 + 102, 54 + 106, 54 + 108, 54 + 110, 55 + 56, 55 + 58, 55 + 60, 55 + 98, 55 + 100, 55 +
102, 55 + 106, 55 + 108, 55 + 110, 56 + 58, 56 + 60, 56 + 98, 56 + 100, 56 + 102, 56 + 106,
56 + 108, 56 + 110, 58 + 60, 58 + 98, 58 + 100, 58 + 102, 58 + 106, 58 + 108, 58 + 110, 60 +
98, 60 + 100, 60 + 102, 60 + 106, 60 + 108, 60 + 110, 98 + 100, 98 + 102, 98 + 106, 98 + 108,
98 + 110, 100 + 102, 100 + 106, 100 + 108, 100 + 110, 102 + 106, 102 + 108, 102 + 110, 106 +
108, 106 + 110, 108 + 110, 27 + 29 + 31, 27 + 29 + 33, 27 + 29 + 34, 27 + 29 + 35, 27 + 29 +
36, 27 + 29 + 37, 27 + 29 + 54, 27 + 29 + 55, 27 + 29 + 56, 27 + 29 + 58, 27 + 29 + 60, 27 +
29 + 98, 27 + 29 + 100, 27 + 29 + 102, 27 + 29 + 106, 27 + 29 + 108, 27 + 29 + 110, 27 + 31 +
33, 27 + 31 + 34, 27 + 31 + 35, 27 + 31 + 36, 27 + 31 + 37, 27 + 31 + 54, 27 + 31 + 55, 27 +
31 + 56, 27 + 31 + 58, 27 + 31 + 60, 27 + 31 + 98, 27 + 31 + 100, 27 + 31 + 102, 27 + 31 +
106, 27 + 31 + 108, 27 + 31 + 110, 27 + 33 + 34, 27 + 33 + 35, 27 + 33 + 36, 27 + 33 + 37, 27 +
33 + 54, 27 + 33 + 55, 27 + 33 + 56, 27 + 33 + 58, 27 + 33 + 60, 27 + 33 + 98, 27 + 33 +
100, 27 + 33 + 102, 27 + 33 + 106, 27 + 33 + 108, 27 + 33 + 110, 27 + 34 + 35, 27 + 34 + 36,
27 + 34 + 37, 27 + 34 + 54, 27 + 34 + 55, 27 + 34 + 56, 27 + 34 + 58, 27 + 34 + 60, 27 + 34 +
98, 27 + 34 + 100, 27 + 34 + 102, 27 + 34 + 106, 27 + 34 + 108, 27 + 34 + 110, 27 + 35 + 36,
27 + 35 + 37, 27 + 35 + 54, 27 + 35 + 55, 27 + 35 + 56, 27 + 35 + 58, 27 + 35 + 60, 27 + 35 +
98, 27 + 35 + 100, 27 + 35 + 102, 27 + 35 + 106, 27 + 35 + 108, 27 + 35 + 110, 27 + 36 + 37,
27 + 36 + 54, 27 + 36 + 55, 27 + 36 + 56, 27 + 36 + 58, 27 + 36 + 60, 27 + 36 + 98, 27 + 36 +
100, 27 + 36 + 102, 27 + 36 + 106, 27 + 36 + 108, 27 + 36 + 110, 27 + 37 + 54, 27 + 37 + 55,
27 + 37 + 56, 27 + 37 + 58, 27 + 37 + 60, 27 + 37 + 98, 27 + 37 + 100, 27 + 37 + 102, 27 + 37 +
106, 27 + 37 + 108, 27 + 37 + 110, 27 + 54 + 55, 27 + 54 + 56, 27 + 54 + 58, 27 + 54 + 60,
27 + 54 + 98, 27 + 54 + 100, 27 + 54 + 102, 27 + 54 + 106, 27 + 54 + 108, 27 + 54 + 110, 27 +
55 + 56, 27 + 55 + 58, 27 + 55 + 60, 27 + 55 + 98, 27 + 55 + 100, 27 + 55 + 102, 27 + 55 +
106, 27 + 55 + 108, 27 + 55 + 110, 27 + 56 + 58, 27 + 56 + 60, 27 + 56 + 98, 27 + 56 + 100,
27 + 56 + 102, 27 + 56 + 106, 27 + 56 + 108, 27 + 56 + 110, 27 + 58 + 60, 27 + 58 + 98, 27 +
58 + 100, 27 + 58 + 102, 27 + 58 + 106, 27 + 58 + 108, 27 + 58 + 110, 27 + 60 + 98, 27 + 60 +
100, 27 + 60 + 102, 27 + 60 + 106, 27 + 60 + 108, 27 + 60 + 110, 27 + 98 + 100, 27 + 98 +
102, 27 + 98 + 106, 27 + 98 + 108, 27 + 98 + 110, 27 + 100 + 102, 27 + 100 + 106, 27 + 100 +
108, 27 + 100 + 110, 27 + 102 + 106, 27 + 102 + 108, 27 + 102 + 110, 27 + 106 + 108, 27 +
106 + 110, 27 + 108 + 110, 29 + 31 + 33, 29 + 31 + 34, 29 + 31 + 35, 29 + 31 + 36, 29 + 31 +
37, 29 + 31 + 54, 29 + 31 + 55, 29 + 31 + 56, 29 + 31 + 58, 29 + 31 + 60, 29 + 31 + 98, 29 +
31 + 100, 29 + 31 + 102, 29 + 31 + 106, 29 + 31 + 108, 29 + 31 + 110, 29 + 33 + 34, 29 + 33 +
35, 29 + 33 + 36, 29 + 33 + 37, 29 + 33 + 54, 29 + 33 + 55, 29 + 33 + 56, 29 + 33 + 58, 29 +
33 + 60, 29 + 33 + 98, 29 + 33 + 100, 29 + 33 + 102, 29 + 33 + 106, 29 + 33 + 108, 29 + 33 +
110, 29 + 34 + 35, 29 + 34 + 36, 29 + 34 + 37, 29 + 34 + 54, 29 + 34 + 55, 29 + 34 + 56, 29 +
34 + 58, 29 + 34 + 60, 29 + 34 + 98, 29 + 34 + 100, 29 + 34 + 102, 29 + 34 + 106, 29 + 34 +
108, 29 + 34 + 110, 29 + 35 + 36, 29 + 35 + 37, 29 + 35 + 54, 29 + 35 + 55, 29 + 35 + 56, 29 +
35 + 58, 29 + 35 + 60, 29 + 35 + 98, 29 + 35 + 100, 29 + 35 + 102, 29 + 35 + 106, 29 + 35 +
108, 29 + 35 + 110, 29 + 36 + 37, 29 + 36 + 54, 29 + 36 + 55, 29 + 36 + 56, 29 + 36 + 58, 29 +
36 + 60, 29 + 36 + 98, 29 + 36 + 100, 29 + 36 + 102, 29 + 36 + 106, 29 + 36 + 108, 29 + 36 +
110, 29 + 37 + 54, 29 + 37 + 55, 29 + 37 + 56, 29 + 37 + 58, 29 + 37 + 60, 29 + 37 + 98, 29 +
37 + 100, 29 + 37 + 102, 29 + 37 + 106, 29 + 37 + 108, 29 + 37 + 110, 29 + 54 + 55, 29 +
54 + 56, 29 + 54 + 58, 29 + 54 + 60, 29 + 54 + 98, 29 + 54 + 100, 29 + 54 + 102, 29 + 54 +
106, 29 + 54 + 108, 29 + 54 + 110, 29 + 55 + 56, 29 + 55 + 58, 29 + 55 + 60, 29 + 55 + 98, 29 +
55 + 100, 29 + 55 + 102, 29 + 55 + 106, 29 + 55 + 108, 29 + 55 + 110, 29 + 56 + 58, 29 +
56 + 60, 29 + 56 + 98, 29 + 56 + 100, 29 + 56 + 102, 29 + 56 + 106, 29 + 56 + 108, 29 + 56 +
110, 29 + 58 + 60, 29 + 58 + 98, 29 + 58 + 100, 29 + 58 + 102, 29 + 58 + 106, 29 + 58 + 108,
29 + 58 + 110, 29 + 60 + 98, 29 + 60 + 100, 29 + 60 + 102, 29 + 60 + 106, 29 + 60 + 108, 29 +
60 + 110, 29 + 98 + 100, 29 + 98 + 102, 29 + 98 + 106, 29 + 98 + 108, 29 + 98 + 110, 29 +
100 + 102, 29 + 100 + 106, 29 + 100 + 108, 29 + 100 + 110, 29 + 102 + 106, 29 + 102 + 108,
29 + 102 + 110, 29 + 106 + 108, 29 + 106 + 110, 29 + 108 + 110, 31 + 33 + 34, 31 + 33 + 35,
31 + 33 + 36, 31 + 33 + 37, 31 + 33 + 54, 31 + 33 + 55, 31 + 33 + 56, 31 + 33 + 58, 31 + 33 +
60, 31 + 33 + 98, 31 + 33 + 100, 31 + 33 + 102, 31 + 33 + 106, 31 + 33 + 108, 31 + 33 + 110,
31 + 34 + 35, 31 + 34 + 36, 31 + 34 + 37, 31 + 34 + 54, 31 + 34 + 55, 31 + 34 + 56, 31 + 34 +
58, 31 + 34 + 60, 31 + 34 + 98, 31 + 34 + 100, 31 + 34 + 102, 31 + 34 + 106, 31 + 34 + 108,
31 + 34 + 110, 31 + 35 + 36, 31 + 35 + 37, 31 + 35 + 54, 31 + 35 + 55, 31 + 35 + 56, 31 + 35 +
58, 31 + 35 + 60, 31 + 35 + 98, 31 + 35 + 100, 31 + 35 + 102, 31 + 35 + 106, 31 + 35 + 108,
31 + 35 + 110, 31 + 36 + 37, 31 + 36 + 54, 31 + 36 + 55, 31 + 36 + 56, 31 + 36 + 58, 31 + 36 +
60, 31 + 36 + 98, 31 + 36 + 100, 31 + 36 + 102, 31 + 36 + 106, 31 + 36 + 108, 31 + 36 +
110, 31 + 37 + 54, 31 + 37 + 55, 31 + 37 + 56, 31 + 37 + 58, 31 + 37 + 60, 31 + 37 + 98, 31 +
37 + 100, 31 + 37 + 102, 31 + 37 + 106, 31 + 37 + 108, 31 + 37 + 110, 31 + 54 + 55, 31 + 54 +
56, 31 + 54 + 58, 31 + 54 + 60, 31 + 54 + 98, 31 + 54 + 100, 31 + 54 + 102, 31 + 54 + 106,
31 + 54 + 108, 31 + 54 + 110, 31 + 55 + 56, 31 + 55 + 58, 31 + 55 + 60, 31 + 55 + 98, 31 + 55 +
100, 31 + 55 + 102, 31 + 55 + 106, 31 + 55 + 108, 31 + 55 + 110, 31 + 56 + 58, 31 + 56 +
60, 31 + 56 + 98, 31 + 56 + 100, 31 + 56 + 102, 31 + 56 + 106, 31 + 56 + 108, 31 + 56 + 110,
31 + 58 + 60, 31 + 58 + 98, 31 + 58 + 100, 31 + 58 + 102, 31 + 58 + 106, 31 + 58 + 108, 31 +
58 + 110, 31 + 60 + 98, 31 + 60 + 100, 31 + 60 + 102, 31 + 60 + 106, 31 + 60 + 108, 31 + 60 +
110, 31 + 98 + 100, 31 + 98 + 102, 31 + 98 + 106, 31 + 98 + 108, 31 + 98 + 110, 31 + 100 +
102, 31 + 100 + 106, 31 + 100 + 108, 31 + 100 + 110, 31 + 102 + 106, 31 + 102 + 108, 31 +
102 + 110, 31 + 106 + 108, 31 + 106 + 110, 31 + 108 + 110, 33 + 34 + 35, 33 + 34 + 36, 33 +
34 + 37, 33 + 34 + 54, 33 + 34 + 55, 33 + 34 + 56, 33 + 34 + 58, 33 + 34 + 60, 33 + 34 + 98,
33 + 34 + 100, 33 + 34 + 102, 33 + 34 + 106, 33 + 34 + 108, 33 + 34 + 110, 33 + 35 + 36, 33 +
35 + 37, 33 + 35 + 54, 33 + 35 + 55, 33 + 35 + 56, 33 + 35 + 58, 33 + 35 + 60, 33 + 35 + 98,
33 + 35 + 100, 33 + 35 + 102, 33 + 35 + 106, 33 + 35 + 108, 33 + 35 + 110, 33 + 36 + 37, 33 +
36 + 54, 33 + 36 + 55, 33 + 36 + 56, 33 + 36 + 58, 33 + 36 + 60, 33 + 36 + 98, 33 + 36 +
100, 33 + 36 + 102, 33 + 36 + 106, 33 + 36 + 108, 33 + 36 + 110, 33 + 37 + 54, 33 + 37 + 55,
33 + 37 + 56, 33 + 37 + 58, 33 + 37 + 60, 33 + 37 + 98, 33 + 37 + 100, 33 + 37 + 102, 33 + 37 +
106, 33 + 37 + 108, 33 + 37 + 110, 33 + 54 + 55, 33 + 54 + 56, 33 + 54 + 58, 33 + 54 + 60,
33 + 54 + 98, 33 + 54 + 100, 33 + 54 + 102, 33 + 54 + 106, 33 + 54 + 108, 33 + 54 + 110, 33 +
55 + 56, 33 + 55 + 58, 33 + 55 + 60, 33 + 55 + 98, 33 + 55 + 100, 33 + 55 + 102, 33 + 55 +
106, 33 + 55 + 108, 33 + 55 + 110, 33 + 56 + 58, 33 + 56 + 60, 33 + 56 + 98, 33 + 56 + 100,
33 + 56 + 102, 33 + 56 + 106, 33 + 56 + 108, 33 + 56 + 110, 33 + 58 + 60, 33 + 58 + 98, 33 +
58 + 100, 33 + 58 + 102, 33 + 58 + 106, 33 + 58 + 108, 33 + 58 + 110, 33 + 60 + 98, 33 + 60 +
100, 33 + 60 + 102, 33 + 60 + 106, 33 + 60 + 108, 33 + 60 + 110, 33 + 98 + 100, 33 + 98 +
102, 33 + 98 + 106, 33 + 98 + 108, 33 + 98 + 110, 33 + 100 + 102, 33 + 100 + 106, 33 + 100 +
108, 33 + 100 + 110, 33 + 102 + 106, 33 + 102 + 108, 33 + 102 + 110, 33 + 106 + 108, 33 +
106 + 110, 33 + 108 + 110, 34 + 35 + 36, 34 + 35 + 37, 34 + 35 + 54, 34 + 35 + 55, 34 + 35 +
56, 34 + 35 + 58, 34 + 35 + 60, 34 + 35 + 98, 34 + 35 + 100, 34 + 35 + 102, 34 + 35 + 106, 34 +
35 + 108, 34 + 35 + 110, 34 + 36 + 37, 34 + 36 + 54, 34 + 36 + 55, 34 + 36 + 56, 34 + 36 +
58, 34 + 36 + 60, 34 + 36 + 98, 34 + 36 + 100, 34 + 36 + 102, 34 + 36 + 106, 34 + 36 + 108,
34 + 36 + 110, 34 + 37 + 54, 34 + 37 + 55, 34 + 37 + 56, 34 + 37 + 58, 34 + 37 + 60, 34 + 37 +
98, 34 + 37 + 100, 34 + 37 + 102, 34 + 37 + 106, 34 + 37 + 108, 34 + 37 + 110, 34 + 54 +
55, 34 + 54 + 56, 34 + 54 + 58, 34 + 54 + 60, 34 + 54 + 98, 34 + 54 + 100, 34 + 54 + 102, 34 +
54 + 106, 34 + 54 + 108, 34 + 54 + 110, 34 + 55 + 56, 34 + 55 + 58, 34 + 55 + 60, 34 + 55 +
98, 34 + 55 + 100, 34 + 55 + 102, 34 + 55 + 106, 34 + 55 + 108, 34 + 55 + 110, 34 + 56 + 58,
34 + 56 + 60, 34 + 56 + 98, 34 + 56 + 100, 34 + 56 + 102, 34 + 56 + 106, 34 + 56 + 108, 34 +
56 + 110, 34 + 58 + 60, 34 + 58 + 98, 34 + 58 + 100, 34 + 58 + 102, 34 + 58 + 106, 34 + 58 +
108, 34 + 58 + 110, 34 + 60 + 98, 34 + 60 + 100, 34 + 60 + 102, 34 + 60 + 106, 34 + 60 + 108,
34 + 60 + 110, 34 + 98 + 100, 34 + 98 + 102, 34 + 98 + 106, 34 + 98 + 108, 34 + 98 + 110, 34 +
100 + 102, 34 + 100 + 106, 34 + 100 + 108, 34 + 100 + 110, 34 + 102 + 106, 34 + 102 +
108, 34 + 102 + 110, 34 + 106 + 108, 34 + 106 + 110, 34 + 108 + 110, 35 + 36 + 37, 35 + 36 +
54, 35 + 36 + 55, 35 + 36 + 56, 35 + 36 + 58, 35 + 36 + 60, 35 + 36 + 98, 35 + 36 + 100, 35 +
36 + 102, 35 + 36 + 106, 35 + 36 + 108, 35 + 36 + 110, 35 + 37 + 54, 35 + 37 + 55, 35 + 37 +
56, 35 + 37 + 58, 35 + 37 + 60, 35 + 37 + 98, 35 + 37 + 100, 35 + 37 + 102, 35 + 37 + 106, 35 +
37 + 108, 35 + 37 + 110, 35 + 54 + 55, 35 + 54 + 56, 35 + 54 + 58, 35 + 54 + 60, 35 + 54 +
98, 35 + 54 + 100, 35 + 54 + 102, 35 + 54 + 106, 35 + 54 + 108, 35 + 54 + 110, 35 + 55 + 56,
35 + 55 + 58, 35 + 55 + 60, 35 + 55 + 98, 35 + 55 + 100, 35 + 55 + 102, 35 + 55 + 106, 35 +
55 + 108, 35 + 55 + 110, 35 + 56 + 58, 35 + 56 + 60, 35 + 56 + 98, 35 + 56 + 100, 35 + 56 +
102, 35 + 56 + 106, 35 + 56 + 108, 35 + 56 + 110, 35 + 58 + 60, 35 + 58 + 98, 35 + 58 + 100,
35 + 58 + 102, 35 + 58 + 106, 35 + 58 + 108, 35 + 58 + 110, 35 + 60 + 98, 35 + 60 + 100, 35 +
60 + 102, 35 + 60 + 106, 35 + 60 + 108, 35 + 60 + 110, 35 + 98 + 100, 35 + 98 + 102, 35 +
98 + 106, 35 + 98 + 108, 35 + 98 + 110, 35 + 100 + 102, 35 + 100 + 106, 35 + 100 + 108, 35 +
100 + 110, 35 + 102 + 106, 35 + 102 + 108, 35 + 102 + 110, 35 + 106 + 108, 35 + 106 + 110,
35 + 108 + 110, 36 + 37 + 54, 36 + 37 + 55, 36 + 37 + 56, 36 + 37 + 58, 36 + 37 + 60, 36 + 37 +
98, 36 + 37 + 100, 36 + 37 + 102, 36 + 37 + 106, 36 + 37 + 108, 36 + 37 + 110, 36 + 54 +
55, 36 + 54 + 56, 36 + 54 + 58, 36 + 54 + 60, 36 + 54 + 98, 36 + 54 + 100, 36 + 54 + 102, 36 +
54 + 106, 36 + 54 + 108, 36 + 54 + 110, 36 + 55 + 56, 36 + 55 + 58, 36 + 55 + 60, 36 + 55 +
98, 36 + 55 + 100, 36 + 55 + 102, 36 + 55 + 106, 36 + 55 + 108, 36 + 55 + 110, 36 + 56 + 58,
36 + 56 + 60, 36 + 56 + 98, 36 + 56 + 100, 36 + 56 + 102, 36 + 56 + 106, 36 + 56 + 108, 36 +
56 + 110, 36 + 58 + 60, 36 + 58 + 98, 36 + 58 + 100, 36 + 58 + 102, 36 + 58 + 106, 36 + 58 +
108, 36 + 58 + 110, 36 + 60 + 98, 36 + 60 + 100, 36 + 60 + 102, 36 + 60 + 106, 36 + 60 + 108,
36 + 60 + 110, 36 + 98 + 100, 36 + 98 + 102, 36 + 98 + 106, 36 + 98 + 108, 36 + 98 + 110, 36 +
100 + 102, 36 + 100 + 106, 36 + 100 + 108, 36 + 100 + 110, 36 + 102 + 106, 36 + 102 +
108, 36 + 102 + 110, 36 + 106 + 108, 36 + 106 + 110, 36 + 108 + 110, 37 + 54 + 55, 37 + 54 +
56, 37 + 54 + 58, 37 + 54 + 60, 37 + 54 + 98, 37 + 54 + 100, 37 + 54 + 102, 37 + 54 + 106, 37 +
54 + 108, 37 + 54 + 110, 37 + 55 + 56, 37 + 55 + 58, 37 + 55 + 60, 37 + 55 + 98, 37 + 55 +
100, 37 + 55 + 102, 37 + 55 + 106, 37 + 55 + 108, 37 + 55 + 110, 37 + 56 + 58, 37 + 56 + 60,
37 + 56 + 98, 37 + 56 + 100, 37 + 56 + 102, 37 + 56 + 106, 37 + 56 + 108, 37 + 56 + 110, 37 +
58 + 60, 37 + 58 + 98, 37 + 58 + 100, 37 + 58 + 102, 37 + 58 + 106, 37 + 58 + 108, 37 + 58 +
110, 37 + 60 + 98, 37 + 60 + 100, 37 + 60 + 102, 37 + 60 + 106, 37 + 60 + 108, 37 + 60 +
110, 37 + 98 + 100, 37 + 98 + 102, 37 + 98 + 106, 37 + 98 + 108, 37 + 98 + 110, 37 + 100 +
102, 37 + 100 + 106, 37 + 100 + 108, 37 + 100 + 110, 37 + 102 + 106, 37 + 102 + 108, 37 +
102 + 110, 37 + 106 + 108, 37 + 106 + 110, 37 + 108 + 110, 54 + 55 + 56, 54 + 55 + 58, 54 +
55 + 60, 54 + 55 + 98, 54 + 55 + 100, 54 + 55 + 102, 54 + 55 + 106, 54 + 55 + 108, 54 + 55 +
110, 54 + 56 + 58, 54 + 56 + 60, 54 + 56 + 98, 54 + 56 + 100, 54 + 56 + 102, 54 + 56 + 106,
54 + 56 + 108, 54 + 56 + 110, 54 + 58 + 60, 54 + 58 + 98, 54 + 58 + 100, 54 + 58 + 102, 54 +
58 + 106, 54 + 58 + 108, 54 + 58 + 110, 54 + 60 + 98, 54 + 60 + 100, 54 + 60 + 102, 54 + 60 +
106, 54 + 60 + 108, 54 + 60 + 110, 54 + 98 + 100, 54 + 98 + 102, 54 + 98 + 106, 54 + 98 +
108, 54 + 98 + 110, 54 + 100 + 102, 54 + 100 + 106, 54 + 100 + 108, 54 + 100 + 110, 54 +
102 + 106, 54 + 102 + 108, 54 + 102 + 110, 54 + 106 + 108, 54 + 106 + 110, 54 + 108 + 110,
55 + 56 + 58, 55 + 56 + 60, 55 + 56 + 98, 55 + 56 + 100, 55 + 56 + 102, 55 + 56 + 106, 55 +
56 + 108, 55 + 56 + 110, 55 + 58 + 60, 55 + 58 + 98, 55 + 58 + 100, 55 + 58 + 102, 55 + 58 +
106, 55 + 58 + 108, 55 + 58 + 110, 55 + 60 + 98, 55 + 60 + 100, 55 + 60 + 102, 55 + 60 + 106,
55 + 60 + 108, 55 + 60 + 110, 55 + 98 + 100, 55 + 98 + 102, 55 + 98 + 106, 55 + 98 + 108, 55 +
98 + 110, 55 + 100 + 102, 55 + 100 + 106, 55 + 100 + 108, 55 + 100 + 110, 55 + 102 + 106,
55 + 102 + 108, 55 + 102 + 110, 55 + 106 + 108, 55 + 106 + 110, 55 + 108 + 110, 56 + 58 +
60, 56 + 58 + 98, 56 + 58 + 100, 56 + 58 + 102, 56 + 58 + 106, 56 + 58 + 108, 56 + 58 + 110,
56 + 60 + 98, 56 + 60 + 100, 56 + 60 + 102, 56 + 60 + 106, 56 + 60 + 108, 56 + 60 + 110, 56 +
98 + 100, 56 + 98 + 102, 56 + 98 + 106, 56 + 98 + 108, 56 + 98 + 110, 56 + 100 + 102, 56 +
100 + 106, 56 + 100 + 108, 56 + 100 + 110, 56 + 102 + 106, 56 + 102 + 108, 56 + 102 + 110,
56 + 106 + 108, 56 + 106 + 110, 56 + 108 + 110, 58 + 60 + 98, 58 + 60 + 100, 58 + 60 + 102,
58 + 60 + 106, 58 + 60 + 108, 58 + 60 + 110, 58 + 98 + 100, 58 + 98 + 102, 58 + 98 + 106, 58 +
98 + 108, 58 + 98 + 110, 58 + 100 + 102, 58 + 100 + 106, 58 + 100 + 108, 58 + 100 + 110,
58 + 102 + 106, 58 + 102 + 108, 58 + 102 + 110, 58 + 106 + 108, 58 + 106 + 110, 58 + 108 +
110, 60 + 98 + 100, 60 + 98 + 102, 60 + 98 + 106, 60 + 98 + 108, 60 + 98 + 110, 60 + 100 +
102, 60 + 100 + 106, 60 + 100 + 108, 60 + 100 + 110, 60 + 102 + 106, 60 + 102 + 108, 60 +
102 + 110, 60 + 106 + 108, 60 + 106 + 110, 60 + 108 + 110, 98 + 100 + 102, 98 + 100 + 106,
98 + 100 + 108, 98 + 100 + 110, 98 + 102 + 106, 98 + 102 + 108, 98 + 102 + 110, 98 + 106 +
108, 98 + 106 + 110, 98 + 108 + 110, 100 + 102 + 106, 100 + 102 + 108, 100 + 102 + 110,
100 + 106 + 108, 100 + 106 + 110, 100 + 108 + 110, 102 + 106 + 108, 102 + 106 + 110, 102 +
108 + 110, 106 + 108 + 110,

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 237, where the light chain variable domain includes a histidine at any of the specific combinations of one or more amino acid positions in SEQ ID NO: 237 listed in Table 6.

TABLE 6
Exemplary Combinations of Amino Acid Positions in SEQ
ID NO: 237 that can be substituted with histidine

25 + 29, 25 + 32, 25 + 33, 25 + 49, 25 + 50, 25 + 90, 25 + 91, 25 + 93, 25 + 95, 29 + 32,
29 + 33, 29 + 49, 29 + 50, 29 + 90, 29 + 91, 29 + 93, 29 + 95, 32 + 33, 32 + 49, 32 + 50,
32 + 90, 32 + 91, 32 + 93, 32 + 95, 33 + 49, 33 + 50, 33 + 90, 33 + 91, 33 + 93, 33 + 95,
49 + 50, 49 + 90, 49 + 91, 49 + 93, 49 + 95, 50 + 90, 50 + 91, 50 + 93, 50 + 95, 90 + 91,
90 + 93, 90 + 95, 91 + 93, 91 + 95, 93 + 95, 25 + 29 + 32, 25 + 29 + 33, 25 + 29 + 49, 25 +
29 + 50, 25 + 29 + 90, 25 + 29 + 91, 25 + 29 + 93, 25 + 29 + 95, 25 + 32 + 33, 25 + 32 +
49, 25 + 32 + 50, 25 + 32 + 90, 25 + 32 + 91, 25 + 32 + 93, 25 + 32 + 95, 25 + 33 + 49, 25 +
33 + 50, 25 + 33 + 90, 25 + 33 + 91, 25 + 33 + 93, 25 + 33 + 95, 25 + 49 + 50, 25 + 49 +
90, 25 + 49 + 91, 25 + 49 + 93, 25 + 49 + 95, 25 + 50 + 90, 25 + 50 + 91, 25 + 50 + 93, 25 +
50 + 95, 25 + 90 + 91, 25 + 90 + 93, 25 + 90 + 95, 25 + 91 + 93, 25 + 91 + 95, 25 + 93 +
95, 29 + 32 + 33, 29 + 32 + 49, 29 + 32 + 50, 29 + 32 + 90, 29 + 32 + 91, 29 + 32 + 93, 29 +
32 + 95, 29 + 33 + 49, 29 + 33 + 50, 29 + 33 + 90, 29 + 33 + 91, 29 + 33 + 93, 29 + 33 +
95, 29 + 49 + 50, 29 + 49 + 90, 29 + 49 + 91, 29 + 49 + 93, 29 + 49 + 95, 29 + 50 + 90, 29 +
50 + 91, 29 + 50 + 93, 29 + 50 + 95, 29 + 90 + 91, 29 + 90 + 93, 29 + 90 + 95, 29 + 91 +
93, 29 + 91 + 95, 29 + 93 + 95, 32 + 33 + 49, 32 + 33 + 50, 32 + 33 + 90, 32 + 33 + 91, 32 +
33 + 93, 32 + 33 + 95, 32 + 49 + 50, 32 + 49 + 90, 32 + 49 + 91, 32 + 49 + 93, 32 + 49 +
95, 32 + 50 + 90, 32 + 50 + 91, 32 + 50 + 93, 32 + 50 + 95, 32 + 90 + 91, 32 + 90 + 93, 32 +
90 + 95, 32 + 91 + 93, 32 + 91 + 95, 32 + 93 + 95, 33 + 49 + 50, 33 + 49 + 90, 33 + 49 +
91, 33 + 49 + 93, 33 + 49 + 95, 33 + 50 + 90, 33 + 50 + 91, 33 + 50 + 93, 33 + 50 + 95, 33 +
90 + 91, 33 + 90 + 93, 33 + 90 + 95, 33 + 91 + 93, 33 + 91 + 95, 33 + 93 + 95, 49 + 50 +
90, 49 + 50 + 91, 49 + 50 + 93, 49 + 50 + 95, 49 + 90 + 91, 49 + 90 + 93, 49 + 90 + 95, 49 +
91 + 93, 49 + 91 + 95, 49 + 93 + 95, 50 + 90 + 91, 50 + 90 + 93, 50 + 90 + 95, 50 + 91 +
93, 50 + 91 + 95, 50 + 93 + 95, 90 + 91 + 93, 90 + 91 + 95, 90 + 93 + 95, 91 + 93 + 95

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5; and a light chain variable domain that that is at least 90% (e.g., at least 92%, at least 94%, at least 96%, at least 98%, at least 99%, or 100%) identical to SEQ ID NO: 237, where the light chain variable domain includes a histidine at any of the specific combinations of one or more amino acid positions in SEQ ID NO: 237 listed in Table 6.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5, and where the heavy chain variable domain includes an alanine at position 52 in SEQ ID NO: 236; and a light chain variable domain that that is at least 90% (e.g., at least 92%, at least 94%, at least 96%, at least 98%, at least 99%, or 100%) identical to SEQ ID NO: 237, where the light chain variable domain includes a histidine at any of the specific combinations of one or more amino acid positions in SEQ ID NO: 237 listed in Table 6.

In some examples of any of the ABPCs described herein, the first antigen-binding domain comprises a light chain variable domain that is at least 90% identical to SEQ ID NO: 237, wherein the light chain variable domain includes a histidine at position 25 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain comprising SEQ ID NO: 237, and a heavy chain variable domain that is at least 90% identical (e.g., at least 92%, at least 94%, at least 96%, at least 98%, at least 99%, or 100%) identical to SEQ ID NO: 236, where the heavy chain variable domain includes a histidine at any of the specific combinations of one or more (e.g., two, three, four, five, six, seven, eight, nine, or ten) amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 29 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 32 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 33 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 49 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 50 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 90 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 91 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 93 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain comprises a light chain variable domain that is at least 90% identical to SEQ ID NO: 237, wherein the light chain variable domain includes a histidine at position 95 in SEQ ID NO: 237; and a heavy chain variable domain that is at least 90% (e.g., at least 92%, at least 94%, at least 96%, at least 98%, at least 99%, or 100%) identical to SEQ ID NO: 236, where the heavy chain variable domain includes a histidine at any of the specific combinations of one or more amino acid positions in SEQ ID NO: 236 listed in Table 5.

In some examples of any of the ABPCs described herein, the first antigen-binding domain includes a heavy chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.13 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.13 comprises SEQ ID NO: 236. In some examples of any of the ABPCs described herein, the light chain variable domain of SC16.13 comprises SEQ ID NO: 237.

In some examples of any of the ABPCs described herein, the first antigen-binding domain comprises a heavy chain variable domain comprising a CDR1, a CDR2, and a CDR3 of SEQ ID NO: 628, SEQ ID NO: 629, and SEQ ID NO: 630, respectively, with collectively a total of one or more (e.g., one, two, three, four, five, six, seven, eight, nine, or ten) histidine(s) in SEQ ID NOs: 628-630 substituted with an alanine. In some examples of any of the ABPCs described herein, the first antigen-binding domain comprises a light chain variable domain comprising a CDR1, a CDR2, and a CDR3 of SEQ ID NO: 631, SEQ ID NO: 632, and SEQ ID NO: 633, respectively, with collectively a total of one or more (e.g., one, two, three, four, five, six, seven, eight, nine, or ten) histidine(s) in SEQ ID NOs: 631-633 substituted with an alanine. In some examples of any of the ABPCs described herein, the first antigen-binding domain includes: a heavy chain variable domain comprising a CDR1, a CDR2, and a CDR3 of SEQ ID NO: 628, SEQ ID NO: 629, and SEQ ID NO: 630, respectively, with collectively a total of one or more (e.g., one, two, three, four, five, six, seven, eight, nine, or ten) histidine(s) in SEQ ID NOs: 628-630 substituted with an alanine; and a light chain variable domain comprising a CDR1, a CDR2, and a CDR3 of SEQ ID NO: 631, SEQ ID NO: 632, and SEQ ID NO: 633, respectively, with collectively a total of one or more (e.g., one, two, three, four, five, six, seven, eight, nine, or ten) histidine(s) in SEQ ID NOs: 631-633 substituted with an alanine.

In some examples of any of the ABPCs described herein, the first antigen-binding domain comprises a heavy chain variable domain of SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain of SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a light chain variable domain comprising SEQ ID NO: 237, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 282, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 283, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 284, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 285, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 286, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 287, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 288, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 289, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 290, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 291, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 292, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 293, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 294, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 295, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 296, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 297, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 298, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 299, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 300, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 301, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 302, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 303, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 304, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 305, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 306, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a light chain variable domain comprising SEQ ID NO: 307, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein, the first antigen-binding domain includes a heavy chain variable domain of SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331. SEQ ID NO: 332, or SEQ ID NO: 333.

In some examples of any of the ABPCs described herein, the first antigen-binding domain includes a heavy chain variable domain comprising SEQ ID NO: 308, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 309, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 310, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 311, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 312, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 313, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 314, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 315, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 316, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 317, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 318, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 319, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 320, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 321, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 322, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 323, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 324, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 325, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 326, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 327, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 328, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 329, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 330, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 331, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 332, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of the ABPCs described herein, the first antigen-binding domain includes a heavy chain variable domain comprising SEQ ID NO: 333, and a light chain variable domain comprising SEQ ID NO: 237, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, or SEQ ID NO: 307.

In some examples of any of ABPCs described herein, the first antigen-binding domain includes a light chain variable domain comprising SEQ ID NO: 334.

In some examples of any of the ABPCs described herein the first antigen-binding domain includes a light chain variable domain comprising SEQ ID NO: 334, and a heavy chain variable domain comprising SEQ ID NO: 236, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, or SEQ ID NO: 281.

In some examples of any of the ABPCs described herein the first antigen-binding domain includes a light chain variable domain comprising SEQ ID NO: 334, and a heavy chain variable domain comprising SEQ ID NO: 239, SEQ ID NO: 241, SEQ ID NO: 243, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 270, SEQ ID NO: 274.

In some examples of any of the ABPCs described herein the first antigen-binding domain includes a light chain variable domain comprising SEQ ID NO: 291 or SEQ ID NO: 292. In some examples of any of the ABPCs described herein the first antigen-binding domain includes a heavy chain variable domain comprising SEQ ID NO: 239, SEQ ID NO: 241, SEQ ID NO: 243, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 270, SEQ ID NO: 274.

In some examples of any of ABPCs described herein the first antigen-binding domain includes a light chain variable domain comprising SEQ ID NO: 291 or SEQ ID NO: 292, and a heavy chain variable domain comprising SEQ ID NO: 239, SEQ ID NO: 241, SEQ ID NO: 243, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 270, SEQ ID NO: 274.

In some examples of any of ABPCs described herein the first antigen-binding domain includes a light chain variable domain comprising SEQ ID NO: 291, and a heavy chain variable domain comprising SEQ ID NO: 239, SEQ ID NO: 241, SEQ ID NO: 243, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 270, SEQ ID NO: 274.

In some examples of any of ABPCs described herein the first antigen-binding domain includes a light chain variable domain comprising SEQ ID NO: 292, and a heavy chain variable domain comprising SEQ ID NO: 239, SEQ ID NO: 241, SEQ ID NO: 243, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 270, SEQ ID NO: 274.

In some examples of any of the ABPCs described herein, the first antigen-binding domain includes a heavy chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.15 comprises SEQ ID NO: 335. In some examples of any of the ABPCs described herein, the light chain variable domain of SC16.15 comprises SEQ ID NO: 336.

In some examples of any of the ABPCs described herein, the first antigen-binding domain comprises a heavy chain variable domain comprising a CDR1, a CDR2, and a CDR3 of SEQ ID NO: 634, SEQ ID NO: 635, and SEQ ID NO: 636, respectively, with collectively a total of one or more (e.g., one, two, three, four, five, six, seven, eight, nine, or ten) amino acid positions in SEQ ID NOs: 634-636 substituted with a histidine. In some examples of any of the ABPCs described herein, the first antigen-binding domain comprises a light chain variable domain comprising a CDR1, a CDR2, and a CDR3 of SEQ ID NO: 637 SEQ ID NO: 638, and SEQ ID NO: 639, respectively, with collectively a total of one or more (e.g., one, two, three, four, five, six, seven, eight, nine, or ten) amino acid positions in SEQ ID NOs: 637-639 substituted with a histidine. In some examples of any of the ABPCs described herein, the first antigen-binding domain includes: a heavy chain variable domain comprising a CDR1, a CDR2, and a CDR3 of SEQ ID NO: 634, SEQ ID NO: 635, and SEQ ID NO: 636, respectively, with collectively a total of one or more (e.g., one, two, three, four, five, six, seven, eight, nine, or ten) amino acid positions in SEQ ID NOs: 634-636 substituted with a histidine; and a light chain variable domain comprising a CDR1, a CDR2, and a CDR3 of SEQ ID NO: 637, SEQ ID NO: 638, and SEQ ID NO: 639, respectively, with collectively a total of one or more (e.g., one, two, three, four, five, six, seven, eight, nine, or ten) amino acid positions in SEQ ID NOs: 637-639 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 335 selected from the group consisting of: 27, 32, 33, 34, 50, 98, 101, 102, 103, 104, and 105. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 336, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 336 selected from the group consisting of: 27, 29, 32, 34, 52, 53, 89, 90, 91, 92, 93, 94, and 96. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 335 selected from the group consisting of: 27, 32, 33, 34, 50, 98, 101, 102, 103, 104, and 105 and a light chain variable domain that is at least 90% (e.g., at least 92%, at least 94%, at least 96%, at least 98%, at least 99%, or 100%) identical to SEQ ID NO: 336, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 336 selected from the group consisting of: 27, 29, 32, 34, 52, 53, 89, 90, 91, 92, 93, 94, and 96.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 335, where the heavy chain variable domain includes an alanine at position 35 in SEQ ID NO: 335.

In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 335, where the heavy chain variable domain includes an alanine at position 35 in SEQ ID NO: 335, and a light chain variable domain that is at least 90% (e.g., at least 92%, at least 94%, at least 96%, at least 98%, at least 99%, or 100%) identical to SEQ ID NO: 336, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 336 selected from the group consisting of: 27, 29, 32, 34, 52, 53, 89, 90, 91, 92, 93, 94, and 96.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 335 selected from the group consisting of: 27, 32, 33, 34, 50, 98, 101, 102, 103, 104, and 105, and where the heavy chain variable domain includes an alanine at position 35 in SEQ ID NO: 335. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 335 selected from the group consisting of: 27, 32, 33, 34, 50, 98, 101, 102, 103, 104, and 105, and where the heavy chain variable domain includes an alanine at position 35 in SEQ ID NO: 335, and a light chain variable domain that is at least 90% (e.g., at least 92%, at least 94%, at least 96%, at least 98%, at least 99%, or 100%) identical to SEQ ID NO: 336, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 336 selected from the group consisting of: 27, 29, 32, 34, 52, 53, 89, 90, 91, 92, 93, 94, and 96.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7 and where the heavy chain variable domain includes an alanine at position 35 of SEQ ID NO: 335.

TABLE 7
Exemplary Combinations of Amino Acid Positions in SEQ
ID NO: 335 that can be substituted with histidine

27 + 32, 27 + 33, 27 + 34, 27 + 50, 27 + 98, 27 + 101, 27 + 102, 27 + 103, 27 + 104, 27 + 105,
32 + 33, 32 + 34, 32 + 50, 32 + 98, 32 + 101, 32 + 102, 32 + 103, 32 + 104, 32 + 105, 33 + 34,
33 + 50, 33 + 98, 33 + 101, 33 + 102, 33 + 103, 33 + 104, 33 + 105, 34 + 50, 34 + 98, 34 +
101, 34 + 102, 34 + 103, 34 + 104, 34 + 105, 50 + 98, 50 + 101, 50 + 102, 50 + 103, 50 + 104,
50 + 105, 98 + 101, 98 + 102, 98 + 103, 98 + 104, 98 + 105, 101 + 102, 101 + 103, 101 + 104,
101 + 105, 102 + 103, 102 + 104, 102 + 105, 103 + 104, 103 + 105, 104 + 105, 27 + 32 + 33,
27 + 32 + 34, 27 + 32 + 50, 27 + 32 + 98, 27 + 32 + 101, 27 + 32 + 102, 27 + 32 + 103, 27 +
32 + 104, 27 + 32 + 105, 27 + 33 + 34, 27 + 33 + 50, 27 + 33 + 98, 27 + 33 + 101, 27 + 33 +
102, 27 + 33 + 103, 27 + 33 + 104, 27 + 33 + 105, 27 + 34 + 50, 27 + 34 + 98, 27 + 34 + 101,
27 + 34 + 102, 27 + 34 + 103, 27 + 34 + 104, 27 + 34 + 105, 27 + 50 + 98, 27 + 50 + 101, 27 +
50 + 102, 27 + 50 + 103, 27 + 50 + 104, 27 + 50 + 105, 27 + 98 + 101, 27 + 98 + 102, 27 +
98 + 103, 27 + 98 + 104, 27 + 98 + 105, 27 + 101 + 102, 27 + 101 + 103, 27 + 101 + 104, 27 +
101 + 105, 27 + 102 + 103, 27 + 102 + 104, 27 + 102 + 105, 27 + 103 + 104, 27 + 103 + 105,
27 + 104 + 105, 32 + 33 + 34, 32 + 33 + 50, 32 + 33 + 98, 32 + 33 + 101, 32 + 33 + 102, 32 +
33 + 103, 32 + 33 + 104, 32 + 33 + 105, 32 + 34 + 50, 32 + 34 + 98, 32 + 34 + 101, 32 + 34 +
102, 32 + 34 + 103, 32 + 34 + 104, 32 + 34 + 105, 32 + 50 + 98, 32 + 50 + 101, 32 + 50 + 102,
32 + 50 + 103, 32 + 50 + 104, 32 + 50 + 105, 32 + 98 + 101, 32 + 98 + 102, 32 + 98 + 103, 32 +
98 + 104, 32 + 98 + 105, 32 + 101 + 102, 32 + 101 + 103, 32 + 101 + 104, 32 + 101 + 105,
32 + 102 + 103, 32 + 102 + 104, 32 + 102 + 105, 32 + 103 + 104, 32 + 103 + 105, 32 + 104 +
105, 33 + 34 + 50, 33 + 34 + 98, 33 + 34 + 101, 33 + 34 + 102, 33 + 34 + 103, 33 + 34 + 104,
33 + 34 + 105, 33 + 50 + 98, 33 + 50 + 101, 33 + 50 + 102, 33 + 50 + 103, 33 + 50 + 104, 33 +
50 + 105, 33 + 98 + 101, 33 + 98 + 102, 33 + 98 + 103, 33 + 98 + 104, 33 + 98 + 105, 33 +
101 + 102, 33 + 101 + 103, 33 + 101 + 104, 33 + 101 + 105, 33 + 102 + 103, 33 + 102 + 104,
33 + 102 + 105, 33 + 103 + 104, 33 + 103 + 105, 33 + 104 + 105, 34 + 50 + 98, 34 + 50 + 101,
34 + 50 + 102, 34 + 50 + 103, 34 + 50 + 104, 34 + 50 + 105, 34 + 98 + 101, 34 + 98 + 102, 34 +
98 + 103, 34 + 98 + 104, 34 + 98 + 105, 34 + 101 + 102, 34 + 101 + 103, 34 + 101 + 104, 34 +
101 + 105, 34 + 102 + 103, 34 + 102 + 104, 34 + 102 + 105, 34 + 103 + 104, 34 + 103 +
105, 34 + 104 + 105, 50 + 98 + 101, 50 + 98 + 102, 50 + 98 + 103, 50 + 98 + 104, 50 + 98 +
105, 50 + 101 + 102, 50 + 101 + 103, 50 + 101 + 104, 50 + 101 + 105, 50 + 102 + 103, 50 +
102 + 104, 50 + 102 + 105, 50 + 103 + 104, 50 + 103 + 105, 50 + 104 + 105, 98 + 101 + 102,
98 + 101 + 103, 98 + 101 + 104, 98 + 101 + 105, 98 + 102 + 103, 98 + 102 + 104, 98 + 102 +
105, 98 + 103 + 104, 98 + 103 + 105, 98 + 104 + 105, 101 + 102 + 103, 101 + 102 + 104, 101 +
102 + 105, 101 + 103 + 104, 101 + 103 + 105, 101 + 104 + 105, 102 + 103 + 104, 102 + 103 +
105, 102 + 104 + 105, 103 + 104 + 105,

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 336, where the light chain variable domain includes a histidine at any of the specific combinations of one or more amino acid positions in SEQ ID NO: 336 listed in Table 8.

TABLE 8
Exemplary Combinations of Amino Acid Positions in SEQ
ID NO: 336 that can be substituted with histidine

27 + 29, 27 + 32, 27 + 34, 27 + 52, 27 + 53, 27 + 89, 27 + 90, 27 + 91, 27 + 92, 27 + 93, 27 +
94, 27 + 96, 29 + 32, 29 + 34, 29 + 52, 29 + 53, 29 + 89, 29 + 90, 29 + 91, 29 + 92, 29 + 93,
29 + 94, 29 + 96, 32 + 34, 32 + 52, 32 + 53, 32 + 89, 32 + 90, 32 + 91, 32 + 92, 32 + 93, 32 +
94, 32 + 96, 34 + 52, 34 + 53, 34 + 89, 34 + 90, 34 + 91, 34 + 92, 34 + 93, 34 + 94, 34 + 96,
52 + 53, 52 + 89, 52 + 90, 52 + 91, 52 + 92, 52 + 93, 52 + 94, 52 + 96, 53 + 89, 53 + 90, 53 +
91, 53 + 92, 53 + 93, 53 + 94, 53 + 96, 89 + 90, 89 + 91, 89 + 92, 89 + 93, 89 + 94, 89 + 96,
90 + 91, 90 + 92, 90 + 93, 90 + 94, 90 + 96, 91 + 92, 91 + 93, 91 + 94, 91 + 96, 92 + 93, 92 +
94, 92 + 96, 93 + 94, 93 + 96, 94 + 96, 27 + 29 + 32, 27 + 29 + 34, 27 + 29 + 52, 27 + 29 +
53, 27 + 29 + 89, 27 + 29 + 90, 27 + 29 + 91, 27 + 29 + 92, 27 + 29 + 93, 27 + 29 + 94, 27 +
29 + 96, 27 + 32 + 34, 27 + 32 + 52, 27 + 32 + 53, 27 + 32 + 89, 27 + 32 + 90, 27 + 32 + 91,
27 + 32 + 92, 27 + 32 + 93, 27 + 32 + 94, 27 + 32 + 96, 27 + 34 + 52, 27 + 34 + 53, 27 + 34 +
89, 27 + 34 + 90, 27 + 34 + 91, 27 + 34 + 92, 27 + 34 + 93, 27 + 34 + 94, 27 + 34 + 96, 27 +
52 + 53, 27 + 52 + 89, 27 + 52 + 90, 27 + 52 + 91, 27 + 52 + 92, 27 + 52 + 93, 27 + 52 + 94,
27 + 52 + 96, 27 + 53 + 89, 27 + 53 + 90, 27 + 53 + 91, 27 + 53 + 92, 27 + 53 + 93, 27 + 53 +
94, 27 + 53 + 96, 27 + 89 + 90, 27 + 89 + 91, 27 + 89 + 92, 27 + 89 + 93, 27 + 89 + 94, 27 +
89 + 96, 27 + 90 + 91, 27 + 90 + 92, 27 + 90 + 93, 27 + 90 + 94, 27 + 90 + 96, 27 + 91 + 92,
27 + 91 + 93, 27 + 91 + 94, 27 + 91 + 96, 27 + 92 + 93, 27 + 92 + 94, 27 + 92 + 96, 27 + 93 +
94, 27 + 93 + 96, 27 + 94 + 96, 29 + 32 + 34, 29 + 32 + 52, 29 + 32 + 53, 29 + 32 + 89, 29 +
32 + 90, 29 + 32 + 91, 29 + 32 + 92, 29 + 32 + 93, 29 + 32 + 94, 29 + 32 + 96, 29 + 34 + 52,
29 + 34 + 53, 29 + 34 + 89, 29 + 34 + 90, 29 + 34 + 91, 29 + 34 + 92, 29 + 34 + 93, 29 + 34 +
94, 29 + 34 + 96, 29 + 52 + 53, 29 + 52 + 89, 29 + 52 + 90, 29 + 52 + 91, 29 + 52 + 92, 29 +
52 + 93, 29 + 52 + 94, 29 + 52 + 96, 29 + 53 + 89, 29 + 53 + 90, 29 + 53 + 91, 29 + 53 + 92,
29 + 53 + 93, 29 + 53 + 94, 29 + 53 + 96, 29 + 89 + 90, 29 + 89 + 91, 29 + 89 + 92, 29 + 89 +
93, 29 + 89 + 94, 29 + 89 + 96, 29 + 90 + 91, 29 + 90 + 92, 29 + 90 + 93, 29 + 90 + 94, 29 +
90 + 96, 29 + 91 + 92, 29 + 91 + 93, 29 + 91 + 94, 29 + 91 + 96, 29 + 92 + 93, 29 + 92 + 94,
29 + 92 + 96, 29 + 93 + 94, 29 + 93 + 96, 29 + 94 + 96, 32 + 34 + 52, 32 + 34 + 53, 32 + 34 +
89, 32 + 34 + 90, 32 + 34 + 91, 32 + 34 + 92, 32 + 34 + 93, 32 + 34 + 94, 32 + 34 + 96, 32 +
52 + 53, 32 + 52 + 89, 32 + 52 + 90, 32 + 52 + 91, 32 + 52 + 92, 32 + 52 + 93, 32 + 52 + 94,
32 + 52 + 96, 32 + 53 + 89, 32 + 53 + 90, 32 + 53 + 91, 32 + 53 + 92, 32 + 53 + 93, 32 + 53 +
94, 32 + 53 + 96, 32 + 89 + 90, 32 + 89 + 91, 32 + 89 + 92, 32 + 89 + 93, 32 + 89 + 94, 32 +
89 + 96, 32 + 90 + 91, 32 + 90 + 92, 32 + 90 + 93, 32 + 90 + 94, 32 + 90 + 96, 32 + 91 + 92,
32 + 91 + 93, 32 + 91 + 94, 32 + 91 + 96, 32 + 92 + 93, 32 + 92 + 94, 32 + 92 + 96, 32 + 93 +
94, 32 + 93 + 96, 32 + 94 + 96, 34 + 52 + 53, 34 + 52 + 89, 34 + 52 + 90, 34 + 52 + 91, 34 +
52 + 92, 34 + 52 + 93, 34 + 52 + 94, 34 + 52 + 96, 34 + 53 + 89, 34 + 53 + 90, 34 + 53 + 91,
34 + 53 + 92, 34 + 53 + 93, 34 + 53 + 94, 34 + 53 + 96, 34 + 89 + 90, 34 + 89 + 91, 34 + 89 +
92, 34 + 89 + 93, 34 + 89 + 94, 34 + 89 + 96, 34 + 90 + 91, 34 + 90 + 92, 34 + 90 + 93, 34 +
90 + 94, 34 + 90 + 96, 34 + 91 + 92, 34 + 91 + 93, 34 + 91 + 94, 34 + 91 + 96, 34 + 92 + 93,
34 + 92 + 94, 34 + 92 + 96, 34 + 93 + 94, 34 + 93 + 96, 34 + 94 + 96, 52 + 53 + 89, 52 + 53 +
90, 52 + 53 + 91, 52 + 53 + 92, 52 + 53 + 93, 52 + 53 + 94, 52 + 53 + 96, 52 + 89 + 90, 52 +
89 + 91, 52 + 89 + 92, 52 + 89 + 93, 52 + 89 + 94, 52 + 89 + 96, 52 + 90 + 91, 52 + 90 + 92,
52 + 90 + 93, 52 + 90 + 94, 52 + 90 + 96, 52 + 91 + 92, 52 + 91 + 93, 52 + 91 + 94, 52 + 91 +
96, 52 + 92 + 93, 52 + 92 + 94, 52 + 92 + 96, 52 + 93 + 94, 52 + 93 + 96, 52 + 94 + 96, 53 +
89 + 90, 53 + 89 + 91, 53 + 89 + 92, 53 + 89 + 93, 53 + 89 + 94, 53 + 89 + 96, 53 + 90 + 91,
53 + 90 + 92, 53 + 90 + 93, 53 + 90 + 94, 53 + 90 + 96, 53 + 91 + 92, 53 + 91 + 93, 53 + 91 +
94, 53 + 91 + 96, 53 + 92 + 93, 53 + 92 + 94, 53 + 92 + 96, 53 + 93 + 94, 53 + 93 + 96, 53 +
94 + 96, 89 + 90 + 91, 89 + 90 + 92, 89 + 90 + 93, 89 + 90 + 94, 89 + 90 + 96, 89 + 91 + 92,
89 + 91 + 93, 89 + 91 + 94, 89 + 91 + 96, 89 + 92 + 93, 89 + 92 + 94, 89 + 92 + 96, 89 + 93 +
94, 89 + 93 + 96, 89 + 94 + 96, 90 + 91 + 92, 90 + 91 + 93, 90 + 91 + 94, 90 + 91 + 96, 90 +
92 + 93, 90 + 92 + 94, 90 + 92 + 96, 90 + 93 + 94, 90 + 93 + 96, 90 + 94 + 96, 91 + 92 + 93,
91 + 92 + 94, 91 + 92 + 96, 91 + 93 + 94, 91 + 93 + 96, 91 + 94 + 96, 92 + 93 + 94, 92 + 93 +
96, 92 + 94 + 96, 93 + 94 + 96,

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7; and a light chain variable domain that that is at least 90% (e.g., at least 92%, at least 94% at least 96%, at least 98%, at least 99%, or 100%) identical to SEQ ID NO: 336, where the light chain variable domain includes a histidine at any of the specific combinations of one or more amino acid positions in SEQ ID NO: 336 listed in Table 8.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7, and where the heavy chain variable domain includes an alanine at position 35 in SEQ ID NO: 335; and a light chain variable domain that that is at least 90% (e.g., at least 92%, at least 94%, at least 96%, at least 98%, at least 99%, or 100%) identical to SEQ ID NO: 336, where the light chain variable domain includes a histidine at any of the specific combinations of one or more amino acid positions in SEQ ID NO: 336 listed in Table 2.

In some examples of any of the ABPCs described herein, the first antigen-binding domain comprises a light chain variable domain that is at least 90% identical to SEQ ID NO: 336, wherein the light chain variable domain includes a histidine at position 27 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain comprising SEQ ID NO: 336, and a heavy chain variable domain that is at least 90% identical (e.g., at least 92%, at least 94%, at least 96%, at least 98%, at least 99%, or 100%) identical to SEQ ID NO: 335, where the heavy chain variable domain includes a histidine at any of the specific combinations of one or more (e.g., two, three, four, five, six, seven, eight, nine, or ten) amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 29 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 32 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 34 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 52 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 53 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 89 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 90 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 91 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 92 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 93 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 94 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain comprises a light chain variable domain that is at least 90% identical to SEQ ID NO: 336, wherein the light chain variable domain includes a histidine at position 96 in SEQ ID NO: 336; and a heavy chain variable domain that is at least 90% (e.g., at least 92%, at least 94%, at least 96%, at least 98%, at least 99%, or 100%) identical to SEQ ID NO: 335, where the heavy chain variable domain includes a histidine at any of the specific combinations of one or more amino acid positions in SEQ ID NO: 335 listed in Table 7.

In some examples of any of the ABPCs described herein, the first antigen-binding domain includes a heavy chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain SC16.15 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.15 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.15 comprises SEQ ID NO: 335. In some examples of any of the ABPCs described herein, the light chain variable domain of SC16.15 comprises SEQ ID NO: 336.

In some examples of any of the ABPCs described herein, the first antigen-binding domain comprises a heavy chain variable domain comprising a CDR1, a CDR2, and a CDR3 of SEQ ID NO: 634, SEQ ID NO: 635, and SEQ ID NO: 636, respectively, with collectively a total of one or more (e.g., one, two, three, four, five, six, seven, eight, nine, or ten) histidine(s) in SEQ ID NOs: 634-636 substituted with an alanine. In some examples of any of the ABPCs described herein, the first antigen-binding domain comprises a light chain variable domain comprising a CDR1, a CDR2, and a CDR3 of SEQ ID NO: 637, SEQ ID NO: 638, and SEQ ID NO: 639, respectively, with collectively a total of one or more (e.g., one, two, three, four, five, six, seven, eight, nine, or ten) histidine(s) in SEQ ID NOs: 637-639 substituted with an alanine. In some examples of any of the ABPCs described herein, the first antigen-binding domain includes: a heavy chain variable domain comprising a CDR1, a CDR2, and a CDR3 of SEQ ID NO: 634, SEQ ID NO: 635, and SEQ ID NO: 636, respectively, with collectively a total of one or more (e.g., one, two, three, four, five, six, seven, eight, nine, or ten) histidine(s) in SEQ ID NOs: 634-636 substituted with an alanine; and a light chain variable domain comprising a CDR1, a CDR2, and a CDR3 of SEQ ID NO: 637, SEQ ID NO: 638, and SEQ ID NO: 639, respectively, with collectively a total of one or more (e.g., one, two, three, four, five, six, seven, eight, nine, or ten) histidine(s) in SEQ ID NOs: 637-639 substituted with an alanine.

In some examples of any of the ABPCs described herein, the first antigen-binding domain comprises a heavy chain variable domain of SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain of SEQ ID NO: 336, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, or SEQ ID NO: 400.

In some examples of any of the ABPCs described herein, the first antigen-binding domain includes a light chain variable domain comprising SEQ ID NO: 336, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 377, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 378, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 379, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 380, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 381, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 382, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 383, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 384, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 385, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 386, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 387, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 388, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 389, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 390, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 391, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 392, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 393, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 394, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 395, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 396, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 397, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 398, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 399, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a light chain variable domain comprising SEQ ID NO: 400, and a heavy chain variable domain comprising SEQ ID NO: 335, SEQ ID NO: 337, SEQ ID NO: 338, SEQ ID NO: 339, SEQ ID NO: SEQ ID NO: 340, SEQ ID NO: 341 SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, or SEQ ID NO: 376.

In some examples of any of the ABPCs described herein, the first antigen-binding domain includes a heavy chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.25 comprises SEQ ID NO: 401. In some examples of any of the ABPCs described herein, the light chain variable domain of SC16.25 comprises SEQ ID NO: 402.

In some examples of any of the ABPCs described herein, the first antigen-binding domain comprises a heavy chain variable domain comprising a CDR1, a CDR2, and a CDR3 of SEQ ID NO: 640, SEQ ID NO: 641, and SEQ ID NO: 642, respectively, with collectively a total of one or more (e.g., one, two, three, four, five, six, seven, eight, nine, or ten) amino acid positions in SEQ ID NOs: 640-642 substituted with a histidine. In some examples of any of the ABPCs described herein, the first antigen-binding domain comprises a light chain variable domain comprising a CDR1, a CDR2, and a CDR3 of SEQ ID NO: 643 SEQ ID NO: 644, and SEQ ID NO: 645, respectively, with collectively a total of one or more (e.g., one, two, three, four, five, six, seven, eight, nine, or ten) amino acid positions in SEQ ID NOs: 643-645 substituted with a histidine. In some examples of any of the ABPCs described herein, the first antigen-binding domain includes: a heavy chain variable domain comprising a CDR1, a CDR2, and a CDR3 of SEQ ID NO: 640, SEQ ID NO: 641, and SEQ ID NO: 642, respectively, with collectively a total of one or more (e.g., one, two, three, four, five, six, seven, eight, nine, or ten) amino acid positions in SEQ ID NOs: 640-642 substituted with a histidine; and a light chain variable domain comprising a CDR1, a CDR2, and a CDR3 of SEQ ID NO: 643, SEQ ID NO: 644, and SEQ ID NO: 645, respectively, with collectively a total of one or more (e.g., one, two, three, four, five, six, seven, eight, nine, or ten) amino acid positions in SEQ ID NOs: 643-645 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 401 selected from the group consisting of: 27, 101, 103, 104, 108, and 109. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 402, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 402 selected from the group consisting of: 30 and 31. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 401 selected from the group consisting of: 27, 101, 103, 104, 108, and 109 and a light chain variable domain that is at least 90% (e.g., at least 92%, at least 94%, at least 96%, at least 98%, at least 99%, or 100%) identical to SEQ ID NO: 402, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 402 selected from the group consisting of: 30 and 31.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at any of the specific combinations of one or more amino acid positions in SEQ ID NO: 401 listed in Table 9.

TABLE 9
Exemplary Combinations of Amino Acid Positions in SEQ
ID NO: 401 that can be substituted with histidine

27 + 101, 27 + 103, 27 + 104, 27 + 108, 27 + 109, 101 + 103, 101 + 104, 101 + 108, 101 +
109, 103 + 104, 103 + 108, 103 + 109, 104 + 108, 104 + 109, 108 + 109, 27 + 101 +
103, 27 + 101 + 104, 27 + 101 + 108, 27 + 101 + 109, 27 + 103 + 104, 27 + 103 + 108, 27 +
103 + 109, 27 + 104 + 108, 27 + 104 + 109, 27 + 108 + 109, 101 + 103 + 104, 101 + 103 +
108, 101 + 103 + 109, 101 + 104 + 108, 101 + 104 + 109, 101 + 108 + 109, 103 + 104 +
108, 103 + 104 + 109, 103 + 108 + 109, 104 + 108 + 109

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 402, where the light chain variable domain includes a histidine at any of the specific combinations of one or more amino acid positions in SEQ ID NO: 402 listed in Table 10.

TABLE 10
Exemplary Combinations of Amino Acid Positions in SEQ
ID NO: 402 that can be substituted with histidine

30 + 31

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at any of the specific combinations of one or more amino acid positions in SEQ ID NO: 401 listed in Table 9; and a light chain variable domain that that is at least 90% (e.g., at least 92%, at least 94%, at least 96%, at least 98%, at least 99%, or 100%) identical to SEQ ID NO: 402, where the light chain variable domain includes a histidine at any of the specific combinations of one or more amino acid positions in SEQ ID NO: 402 listed in Table 10.

In some examples of any of the ABPCs described herein, the first antigen-binding domain comprises a light chain variable domain that is at least 90% identical to SEQ ID NO: 402, wherein the light chain variable domain includes a histidine at position 30 in SEQ ID NO: 402; and a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at any of the specific combinations of one or more amino acid positions in SEQ ID NO: 401 listed in Table 9.

In some examples of any of the ABPCs described herein, the first antigen-binding domain comprises a light chain variable domain comprising SEQ ID NO: 402, and a heavy chain variable domain that is at least 90% identical (e.g., at least 92%, at least 94%, at least 96%, at least 98%, at least 99%, or 100%) identical to SEQ ID NO: 401, where the heavy chain variable domain includes a histidine at any of the specific combinations of one or more (e.g., two, three, four, five, six, seven, eight, nine, or ten) amino acid positions in SEQ ID NO: 401 listed in Table 9.

In some examples of any of the ABPCs described herein, the first antigen-binding domain comprises a light chain variable domain that is at least 90% identical to SEQ ID NO: 402, wherein the light chain variable domain includes a histidine at position 31 in SEQ ID NO: 402; and a heavy chain variable domain that is at least 90% (e.g., at least 92%, at least 94%, at least 96%, at least 98%, at least 99%, or 100%) identical to SEQ ID NO: 401, where the heavy chain variable domain includes a histidine at any of the specific combinations of one or more amino acid positions in SEQ ID NO: 401 listed in Table 9.

In some examples of any of the ABPCs described herein, the first antigen-binding domain includes a heavy chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain SC16.25 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.25 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.25 comprises SEQ ID NO: 401. In some examples of any of the ABPCs described herein, the light chain variable domain of SC16.25 comprises SEQ ID NO: 402.

In some examples of any of the ABPCs described herein, the first antigen-binding domain comprises a heavy chain variable domain comprising a CDR1, a CDR2, and a CDR3 of SEQ ID NO: 640, SEQ ID NO: 641, and SEQ ID NO: 642, respectively, with collectively a total of one or more (e.g., one, two, three, four, five, six, seven, eight, nine, or ten) histidine(s) in SEQ ID NOs: 640-642 substituted with an alanine. In some examples of any of the ABPCs described herein, the first antigen-binding domain comprises a light chain variable domain comprising a CDR1, a CDR2, and a CDR3 of SEQ ID NO: 643, SEQ ID NO: 644, and SEQ ID NO: 645, respectively, with collectively a total of one or more (e.g., one, two, three, four, five, six, seven, eight, nine, or ten) histidine(s) in SEQ ID NOs: 643-645 substituted with an alanine. In some examples of any of the ABPCs described herein, the first antigen-binding domain includes: a heavy chain variable domain comprising a CDR1, a CDR2, and a CDR3 of SEQ ID NO: 640, SEQ ID NO: 641, and SEQ ID NO: 642, respectively, with collectively a total of one or more (e.g., one, two, three, four, five, six, seven, eight, nine, or ten) histidine(s) in SEQ ID NOs: 640-642 substituted with an alanine; and a light chain variable domain comprising a CDR1, a CDR2, and a CDR3 of SEQ ID NO: 643, SEQ ID NO: 644, and SEQ ID NO: 645, respectively, with collectively a total of one or more (e.g., one, two, three, four, five, six, seven, eight, nine, or ten) histidine(s) in SEQ ID NOs: 643-645 substituted with an alanine.

In some examples of any of the ABPCs described herein, the first antigen-binding domain comprises a heavy chain variable domain of SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain of SEQ ID NO: 402, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, or SEQ ID NO: 469.

In some examples of any of the ABPCs described herein, the first antigen-binding domain includes a light chain variable domain comprising SEQ ID NO: 402, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 447, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 448, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 449, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 450, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 451, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 452, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 453, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 454, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 455, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 456, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 457, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 458, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 459, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 460, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 461, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 462, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 463, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 464, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 465, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 466, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 467, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 468, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a light chain variable domain comprising SEQ ID NO: 469, and a heavy chain variable domain comprising SEQ ID NO: 401, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, or SEQ ID NO: 446.

In some examples of any of the ABPCs described herein, the first antigen-binding domain includes a heavy chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.34 comprises SEQ ID NO: 470. In some examples of any of the ABPCs described herein, the light chain variable domain of SC16.34 comprises SEQ ID NO: 471.

In some examples of any of the ABPCs described herein, the first antigen-binding domain comprises a heavy chain variable domain comprising a CDR1, a CDR2, and a CDR3 of SEQ ID NO: 646, SEQ ID NO: 647, and SEQ ID NO: 648, respectively, with collectively a total of one or more (e.g., one, two, three, four, five, six, seven, eight, nine, or ten) amino acid positions in SEQ ID NOs: 646-648 substituted with a histidine. In some examples of any of the ABPCs described herein, the first antigen-binding domain comprises a light chain variable domain comprising a CDR1, a CDR2, and a CDR3 of SEQ ID NO: 649 SEQ ID NO: 650, and SEQ ID NO: 651, respectively, with collectively a total of one or more (e.g., one, two, three, four, five, six, seven, eight, nine, or ten) amino acid positions in SEQ ID NOs: 649-651 substituted with a histidine. In some examples of any of the ABPCs described herein, the first antigen-binding domain includes: a heavy chain variable domain comprising a CDR1, a CDR2, and a CDR3 of SEQ ID NO: 646, SEQ ID NO: 647, and SEQ ID NO: 648, respectively, with collectively a total of one or more (e.g., one, two, three, four, five, six, seven, eight, nine, or ten) amino acid positions in SEQ ID NOs: 646-648 substituted with a histidine; and a light chain variable domain comprising a CDR1, a CDR2, and a CDR3 of SEQ ID NO: 649, SEQ ID NO: 650, and SEQ ID NO: 651, respectively, with collectively a total of one or more (e.g., one, two, three, four, five, six, seven, eight, nine, or ten) amino acid positions in SEQ ID NOs: 649-651 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 470 selected from the group consisting of: 24, 27, 29, 32, 34, 35, 50, 51, 53, 54, 56, 58, 59, 60, 63, 98, 101, 103, 105, 106, and 107. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 471, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 471 selected from the group consisting of: 29, 32, 33, 34, 50, 51, 53, 55, 89, 92, 94, 96 and 97. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 470 selected from the group consisting of: 24, 27, 29, 32, 34, 35, 50, 51, 53, 54, 56, 58, 59, 60, 63, 98, 101, 103, 105, 106, and 107 and a light chain variable domain that is at least 90% (e.g., at least 92%, at least 94%, at least 96%, at least 98%, at least 99%, or 100%) identical to SEQ ID NO: 471, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 471 selected from the group consisting of: 29, 32, 33, 34, 50, 51, 53, 55, 89, 92, 94, 96 and 97.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

TABLE 11
Exemplary Combinations of Amino Acid Positions in SEQ
ID NO: 470 that can be substituted with histidine

24 + 27, 24 + 29, 24 + 32, 24 + 34, 24 + 35, 24 + 50, 24 + 51, 24 + 53, 24 + 54, 24 + 56, 24 +
58, 24 + 59, 24 + 60, 24 + 63, 24 + 98, 24 + 101, 24 + 103, 24 + 105, 24 + 106, 24 + 107, 27 +
29, 27 + 32, 27 + 34, 27 + 35, 27 + 50, 27 + 51, 27 + 53, 27 + 54, 27 + 56, 27 + 58, 27 + 59,
27 + 60, 27 + 63, 27 + 98, 27 + 101, 27 + 103, 27 + 105, 27 + 106, 27 + 107, 29 + 32, 29 + 34,
29 + 35, 29 + 50, 29 + 51, 29 + 53, 29 + 54, 29 + 56, 29 + 58, 29 + 59, 29 + 60, 29 + 63, 29 +
98, 29 + 101, 29 + 103, 29 + 105, 29 + 106, 29 + 107, 32 + 34, 32 + 35, 32 + 50, 32 + 51, 32 +
53, 32 + 54, 32 + 56, 32 + 58, 32 + 59, 32 + 60, 32 + 63, 32 + 98, 32 + 101, 32 + 103, 32 +
105, 32 + 106, 32 + 107, 34 + 35, 34 + 50, 34 + 51, 34 + 53, 34 + 54, 34 + 56, 34 + 58, 34 +
59, 34 + 60, 34 + 63, 34 + 98, 34 + 101, 34 + 103, 34 + 105, 34 + 106, 34 + 107, 35 + 50, 35 +
51, 35 + 53, 35 + 54, 35 + 56, 35 + 58, 35 + 59, 35 + 60, 35 + 63, 35 + 98, 35 + 101, 35 + 103,
35 + 105, 35 + 106, 35 + 107, 50 + 51, 50 + 53, 50 + 54, 50 + 56, 50 + 58, 50 + 59, 50 + 60,
50 + 63, 50 + 98, 50 + 101, 50 + 103, 50 + 105, 50 + 106, 50 + 107, 51 + 53, 51 + 54, 51 + 56,
51 + 58, 51 + 59, 51 + 60, 51 + 63, 51 + 98, 51 + 101, 51 + 103, 51 + 105, 51 + 106, 51 + 107,
53 + 54, 53 + 56, 53 + 58, 53 + 59, 53 + 60, 53 + 63, 53 + 98, 53 + 101, 53 + 103, 53 + 105,
53 + 106, 53 + 107, 54 + 56, 54 + 58, 54 + 59, 54 + 60, 54 + 63, 54 + 98, 54 + 101, 54 + 103,
54 + 105, 54 + 106, 54 + 107, 56 + 58, 56 + 59, 56 + 60, 56 + 63, 56 + 98, 56 + 101, 56 + 103,
56 + 105, 56 + 106, 56 + 107, 58 + 59, 58 + 60, 58 + 63, 58 + 98, 58 + 101, 58 + 103, 58 +
105, 58 + 106, 58 + 107, 59 + 60, 59 + 63, 59 + 98, 59 + 101, 59 + 103, 59 + 105, 59 + 106,
59 + 107, 60 + 63, 60 + 98, 60 + 101, 60 + 103, 60 + 105, 60 + 106, 60 + 107, 63 + 98, 63 +
101, 63 + 103, 63 + 105, 63 + 106, 63 + 107, 98 + 101, 98 + 103, 98 + 105, 98 + 106, 98 +
107, 101 + 103, 101 + 105, 101 + 106, 101 + 107, 103 + 105, 103 + 106, 103 + 107, 105 +
106, 105 + 107, 106 + 107, 24 + 27 + 29, 24 + 27 + 32, 24 + 27 + 34, 24 + 27 + 35, 24 + 27 +
50, 24 + 27 + 51, 24 + 27 + 53, 24 + 27 + 54, 24 + 27 + 56, 24 + 27 + 58, 24 + 27 + 59, 24 +
27 + 60, 24 + 27 + 63, 24 + 27 + 98, 24 + 27 + 101, 24 + 27 + 103, 24 + 27 + 105, 24 + 27 +
106, 24 + 27 + 107, 24 + 29 + 32, 24 + 29 + 34, 24 + 29 + 35, 24 + 29 + 50, 24 + 29 + 51, 24 +
29 + 53, 24 + 29 + 54, 24 + 29 + 56, 24 + 29 + 58, 24 + 29 + 59, 24 + 29 + 60, 24 + 29 + 63,
24 + 29 + 98, 24 + 29 + 101, 24 + 29 + 103, 24 + 29 + 105, 24 + 29 + 106, 24 + 29 + 107, 24 +
32 + 34, 24 + 32 + 35, 24 + 32 + 50, 24 + 32 + 51, 24 + 32 + 53, 24 + 32 + 54, 24 + 32 + 56,
24 + 32 + 58, 24 + 32 + 59, 24 + 32 + 60, 24 + 32 + 63, 24 + 32 + 98, 24 + 32 + 101, 24 + 32 +
103, 24 + 32 + 105, 24 + 32 + 106, 24 + 32 + 107, 24 + 34 + 35, 24 + 34 + 50, 24 + 34 + 51,
24 + 34 + 53, 24 + 34 + 54, 24 + 34 + 56, 24 + 34 + 58, 24 + 34 + 59, 24 + 34 + 60, 24 + 34 +
63, 24 + 34 + 98, 24 + 34 + 101, 24 + 34 + 103, 24 + 34 + 105, 24 + 34 + 106, 24 + 34 + 107,
24 + 35 + 50, 24 + 35 + 51, 24 + 35 + 53, 24 + 35 + 54, 24 + 35 + 56, 24 + 35 + 58, 24 + 35 +
59, 24 + 35 + 60, 24 + 35 + 63, 24 + 35 + 98, 24 + 35 + 101, 24 + 35 + 103, 24 + 35 + 105, 24 +
35 + 106, 24 + 35 + 107, 24 + 50 + 51, 24 + 50 + 53, 24 + 50 + 54, 24 + 50 + 56, 24 + 50 +
58, 24 + 50 + 59, 24 + 50 + 60, 24 + 50 + 63, 24 + 50 + 98, 24 + 50 + 101, 24 + 50 + 103, 24 +
50 + 105, 24 + 50 + 106, 24 + 50 + 107, 24 + 51 + 53, 24 + 51 + 54, 24 + 51 + 56, 24 + 51 +
58, 24 + 51 + 59, 24 + 51 + 60, 24 + 51 + 63, 24 + 51 + 98, 24 + 51 + 101, 24 + 51 + 103, 24 +
51 + 105, 24 + 51 + 106, 24 + 51 + 107, 24 + 53 + 54, 24 + 53 + 56, 24 + 53 + 58, 24 + 53 +
59, 24 + 53 + 60, 24 + 53 + 63, 24 + 53 + 98, 24 + 53 + 101, 24 + 53 + 103, 24 + 53 + 105, 24 +
53 + 106, 24 + 53 + 107, 24 + 54 + 56, 24 + 54 + 58, 24 + 54 + 59, 24 + 54 + 60, 24 + 54 +
63, 24 + 54 + 98, 24 + 54 + 101, 24 + 54 + 103, 24 + 54 + 105, 24 + 54 + 106, 24 + 54 + 107,
24 + 56 + 58, 24 + 56 + 59, 24 + 56 + 60, 24 + 56 + 63, 24 + 56 + 98, 24 + 56 + 101, 24 + 56 +
103, 24 + 56 + 105, 24 + 56 + 106, 24 + 56 + 107, 24 + 58 + 59, 24 + 58 + 60, 24 + 58 + 63,
24 + 58 + 98, 24 + 58 + 101, 24 + 58 + 103, 24 + 58 + 105, 24 + 58 + 106, 24 + 58 + 107, 24 +
59 + 60, 24 + 59 + 63, 24 + 59 + 98, 24 + 59 + 101, 24 + 59 + 103, 24 + 59 + 105, 24 + 59 +
106, 24 + 59 + 107, 24 + 60 + 63, 24 + 60 + 98, 24 + 60 + 101, 24 + 60 + 103, 24 + 60 + 105,
24 + 60 + 106, 24 + 60 + 107, 24 + 63 + 98, 24 + 63 + 101, 24 + 63 + 103, 24 + 63 + 105, 24 +
63 + 106, 24 + 63 + 107, 24 + 98 + 101, 24 + 98 + 103, 24 + 98 + 105, 24 + 98 + 106, 24 +
98 + 107, 24 + 101 + 103, 24 + 101 + 105, 24 + 101 + 106, 24 + 101 + 107, 24 + 103 + 105,
24 + 103 + 106, 24 + 103 + 107, 24 + 105 + 106, 24 + 105 + 107, 24 + 106 + 107, 27 + 29 +
32, 27 + 29 + 34, 27 + 29 + 35, 27 + 29 + 50, 27 + 29 + 51, 27 + 29 + 53, 27 + 29 + 54, 27 +
29 + 56, 27 + 29 + 58, 27 + 29 + 59, 27 + 29 + 60, 27 + 29 + 63, 27 + 29 + 98, 27 + 29 + 101,
27 + 29 + 103, 27 + 29 + 105, 27 + 29 + 106, 27 + 29 + 107, 27 + 32 + 34, 27 + 32 + 35, 27 +
32 + 50, 27 + 32 + 51, 27 + 32 + 53, 27 + 32 + 54, 27 + 32 + 56, 27 + 32 + 58, 27 + 32 + 59,
27 + 32 + 60, 27 + 32 + 63, 27 + 32 + 98, 27 + 32 + 101, 27 + 32 + 103, 27 + 32 + 105, 27 +
32 + 106, 27 + 32 + 107, 27 + 34 + 35, 27 + 34 + 50, 27 + 34 + 51, 27 + 34 + 53, 27 + 34 +
54, 27 + 34 + 56, 27 + 34 + 58, 27 + 34 + 59, 27 + 34 + 60, 27 + 34 + 63, 27 + 34 + 98, 27 +
34 + 101, 27 + 34 + 103, 27 + 34 + 105, 27 + 34 + 106, 27 + 34 + 107, 27 + 35 + 50, 27 + 35 +
51, 27 + 35 + 53, 27 + 35 + 54, 27 + 35 + 56, 27 + 35 + 58, 27 + 35 + 59, 27 + 35 + 60, 27 +
35 + 63, 27 + 35 + 98, 27 + 35 + 101, 27 + 35 + 103, 27 + 35 + 105, 27 + 35 + 106, 27 + 35 +
107, 27 + 50 + 51, 27 + 50 + 53, 27 + 50 + 54, 27 + 50 + 56, 27 + 50 + 58, 27 + 50 + 59, 27 +
50 + 60, 27 + 50 + 63, 27 + 50 + 98, 27 + 50 + 101, 27 + 50 + 103, 27 + 50 + 105, 27 + 50 +
106, 27 + 50 + 107, 27 + 51 + 53, 27 + 51 + 54, 27 + 51 + 56, 27 + 51 + 58, 27 + 51 + 59, 27 +
51 + 60, 27 + 51 + 63, 27 + 51 + 98, 27 + 51 + 101, 27 + 51 + 103, 27 + 51 + 105, 27 + 51 +
106, 27 + 51 + 107, 27 + 53 + 54, 27 + 53 + 56, 27 + 53 + 58, 27 + 53 + 59, 27 + 53 + 60, 27 +
53 + 63, 27 + 53 + 98, 27 + 53 + 101, 27 + 53 + 103, 27 + 53 + 105, 27 + 53 + 106, 27 + 53 +
107, 27 + 54 + 56, 27 + 54 + 58, 27 + 54 + 59, 27 + 54 + 60, 27 + 54 + 63, 27 + 54 + 98, 27 +
54 + 101, 27 + 54 + 103, 27 + 54 + 105, 27 + 54 + 106, 27 + 54 + 107, 27 + 56 + 58, 27 +
56 + 59, 27 + 56 + 60, 27 + 56 + 63, 27 + 56 + 98, 27 + 56 + 101, 27 + 56 + 103, 27 + 56 +
105, 27 + 56 + 106, 27 + 56 + 107, 27 + 58 + 59, 27 + 58 + 60, 27 + 58 + 63, 27 + 58 + 98, 27 +
58 + 101, 27 + 58 + 103, 27 + 58 + 105, 27 + 58 + 106, 27 + 58 + 107, 27 + 59 + 60, 27 +
59 + 63, 27 + 59 + 98, 27 + 59 + 101, 27 + 59 + 103, 27 + 59 + 105, 27 + 59 + 106, 27 + 59 +
107, 27 + 60 + 63, 27 + 60 + 98, 27 + 60 + 101, 27 + 60 + 103, 27 + 60 + 105, 27 + 60 + 106,
27 + 60 + 107, 27 + 63 + 98, 27 + 63 + 101, 27 + 63 + 103, 27 + 63 + 105, 27 + 63 + 106, 27 +
63 + 107, 27 + 98 + 101, 27 + 98 + 103, 27 + 98 + 105, 27 + 98 + 106, 27 + 98 + 107, 27 +
101 + 103, 27 + 101 + 105, 27 + 101 + 106, 27 + 101 + 107, 27 + 103 + 105, 27 + 103 + 106,
27 + 103 + 107, 27 + 105 + 106, 27 + 105 + 107, 27 + 106 + 107, 29 + 32 + 34, 29 + 32 + 35,
29 + 32 + 50, 29 + 32 + 51, 29 + 32 + 53, 29 + 32 + 54, 29 + 32 + 56, 29 + 32 + 58, 29 + 32 +
59, 29 + 32 + 60, 29 + 32 + 63, 29 + 32 + 98, 29 + 32 + 101, 29 + 32 + 103, 29 + 32 + 105, 29 +
32 + 106, 29 + 32 + 107, 29 + 34 + 35, 29 + 34 + 50, 29 + 34 + 51, 29 + 34 + 53, 29 + 34 +
54, 29 + 34 + 56, 29 + 34 + 58, 29 + 34 + 59, 29 + 34 + 60, 29 + 34 + 63, 29 + 34 + 98, 29 +
34 + 101, 29 + 34 + 103, 29 + 34 + 105, 29 + 34 + 106, 29 + 34 + 107, 29 + 35 + 50, 29 + 35 +
51, 29 + 35 + 53, 29 + 35 + 54, 29 + 35 + 56, 29 + 35 + 58, 29 + 35 + 59, 29 + 35 + 60, 29 +
35 + 63, 29 + 35 + 98, 29 + 35 + 101, 29 + 35 + 103, 29 + 35 + 105, 29 + 35 + 106, 29 + 35 +
107, 29 + 50 + 51, 29 + 50 + 53, 29 + 50 + 54, 29 + 50 + 56, 29 + 50 + 58, 29 + 50 + 59, 29 +
50 + 60, 29 + 50 + 63, 29 + 50 + 98, 29 + 50 + 101, 29 + 50 + 103, 29 + 50 + 105, 29 + 50 +
106, 29 + 50 + 107, 29 + 51 + 53, 29 + 51 + 54, 29 + 51 + 56, 29 + 51 + 58, 29 + 51 + 59, 29 +
51 + 60, 29 + 51 + 63, 29 + 51 + 98, 29 + 51 + 101, 29 + 51 + 103, 29 + 51 + 105, 29 + 51 +
106, 29 + 51 + 107, 29 + 53 + 54, 29 + 53 + 56, 29 + 53 + 58, 29 + 53 + 59, 29 + 53 + 60, 29 +
53 + 63, 29 + 53 + 98, 29 + 53 + 101, 29 + 53 + 103, 29 + 53 + 105, 29 + 53 + 106, 29 + 53 +
107, 29 + 54 + 56, 29 + 54 + 58, 29 + 54 + 59, 29 + 54 + 60, 29 + 54 + 63, 29 + 54 + 98, 29 +
54 + 101, 29 + 54 + 103, 29 + 54 + 105, 29 + 54 + 106, 29 + 54 + 107, 29 + 56 + 58, 29 +
56 + 59, 29 + 56 + 60, 29 + 56 + 63, 29 + 56 + 98, 29 + 56 + 101, 29 + 56 + 103, 29 + 56 +
105, 29 + 56 + 106, 29 + 56 + 107, 29 + 58 + 59, 29 + 58 + 60, 29 + 58 + 63, 29 + 58 + 98, 29 +
58 + 101, 29 + 58 + 103, 29 + 58 + 105, 29 + 58 + 106, 29 + 58 + 107, 29 + 59 + 60, 29 +
59 + 63, 29 + 59 + 98, 29 + 59 + 101, 29 + 59 + 103, 29 + 59 + 105, 29 + 59 + 106, 29 + 59 +
107, 29 + 60 + 63, 29 + 60 + 98, 29 + 60 + 101, 29 + 60 + 103, 29 + 60 + 105, 29 + 60 + 106,
29 + 60 + 107, 29 + 63 + 98, 29 + 63 + 101, 29 + 63 + 103, 29 + 63 + 105, 29 + 63 + 106, 29 +
63 + 107, 29 + 98 + 101, 29 + 98 + 103, 29 + 98 + 105, 29 + 98 + 106, 29 + 98 + 107, 29 +
101 + 103, 29 + 101 + 105, 29 + 101 + 106, 29 + 101 + 107, 29 + 103 + 105, 29 + 103 + 106,
29 + 103 + 107, 29 + 105 + 106, 29 + 105 + 107, 29 + 106 + 107, 32 + 34 + 35, 32 + 34 + 50,
32 + 34 + 51, 32 + 34 + 53, 32 + 34 + 54, 32 + 34 + 56, 32 + 34 + 58, 32 + 34 + 59, 32 + 34 +
60, 32 + 34 + 63, 32 + 34 + 98, 32 + 34 + 101, 32 + 34 + 103, 32 + 34 + 105, 32 + 34 + 106,
32 + 34 + 107, 32 + 35 + 50, 32 + 35 + 51, 32 + 35 + 53, 32 + 35 + 54, 32 + 35 + 56, 32 + 35 +
58, 32 + 35 + 59, 32 + 35 + 60, 32 + 35 + 63, 32 + 35 + 98, 32 + 35 + 101, 32 + 35 + 103,
32 + 35 + 105, 32 + 35 + 106, 32 + 35 + 107, 32 + 50 + 51, 32 + 50 + 53, 32 + 50 + 54, 32 +
50 + 56, 32 + 50 + 58, 32 + 50 + 59, 32 + 50 + 60, 32 + 50 + 63, 32 + 50 + 98, 32 + 50 + 101,
32 + 50 + 103, 32 + 50 + 105, 32 + 50 + 106, 32 + 50 + 107, 32 + 51 + 53, 32 + 51 + 54, 32 +
51 + 56, 32 + 51 + 58, 32 + 51 + 59, 32 + 51 + 60, 32 + 51 + 63, 32 + 51 + 98, 32 + 51 + 101,
32 + 51 + 103, 32 + 51 + 105, 32 + 51 + 106, 32 + 51 + 107, 32 + 53 + 54, 32 + 53 + 56, 32 +
53 + 58, 32 + 53 + 59, 32 + 53 + 60, 32 + 53 + 63, 32 + 53 + 98, 32 + 53 + 101, 32 + 53 +
103, 32 + 53 + 105, 32 + 53 + 106, 32 + 53 + 107, 32 + 54 + 56, 32 + 54 + 58, 32 + 54 + 59,
32 + 54 + 60, 32 + 54 + 63, 32 + 54 + 98, 32 + 54 + 101, 32 + 54 + 103, 32 + 54 + 105, 32 +
54 + 106, 32 + 54 + 107, 32 + 56 + 58, 32 + 56 + 59, 32 + 56 + 60, 32 + 56 + 63, 32 + 56 +
98, 32 + 56 + 101, 32 + 56 + 103, 32 + 56 + 105, 32 + 56 + 106, 32 + 56 + 107, 32 + 58 + 59,
32 + 58 + 60, 32 + 58 + 63, 32 + 58 + 98, 32 + 58 + 101, 32 + 58 + 103, 32 + 58 + 105, 32 +
58 + 106, 32 + 58 + 107, 32 + 59 + 60, 32 + 59 + 63, 32 + 59 + 98, 32 + 59 + 101, 32 + 59 +
103, 32 + 59 + 105, 32 + 59 + 106, 32 + 59 + 107, 32 + 60 + 63, 32 + 60 + 98, 32 + 60 + 101,
32 + 60 + 103, 32 + 60 + 105, 32 + 60 + 106, 32 + 60 + 107, 32 + 63 + 98, 32 + 63 + 101, 32 +
63 + 103, 32 + 63 + 105, 32 + 63 + 106, 32 + 63 + 107, 32 + 98 + 101, 32 + 98 + 103, 32 +
98 + 105, 32 + 98 + 106, 32 + 98 + 107, 32 + 101 + 103, 32 + 101 + 105, 32 + 101 + 106, 32 +
101 + 107, 32 + 103 + 105, 32 + 103 + 106, 32 + 103 + 107, 32 + 105 + 106, 32 + 105 + 107,
32 + 106 + 107, 34 + 35 + 50, 34 + 35 + 51, 34 + 35 + 53, 34 + 35 + 54, 34 + 35 + 56, 34 + 35 +
58, 34 + 35 + 59, 34 + 35 + 60, 34 + 35 + 63, 34 + 35 + 98, 34 + 35 + 101, 34 + 35 + 103,
34 + 35 + 105, 34 + 35 + 106, 34 + 35 + 107, 34 + 50 + 51, 34 + 50 + 53, 34 + 50 + 54, 34 +
50 + 56, 34 + 50 + 58, 34 + 50 + 59, 34 + 50 + 60, 34 + 50 + 63, 34 + 50 + 98, 34 + 50 + 101,
34 + 50 + 103, 34 + 50 + 105, 34 + 50 + 106, 34 + 50 + 107, 34 + 51 + 53, 34 + 51 + 54, 34 +
51 + 56, 34 + 51 + 58, 34 + 51 + 59, 34 + 51 + 60, 34 + 51 + 63, 34 + 51 + 98, 34 + 51 + 101,
34 + 51 + 103, 34 + 51 + 105, 34 + 51 + 106, 34 + 51 + 107, 34 + 53 + 54, 34 + 53 + 56, 34 +
53 + 58, 34 + 53 + 59, 34 + 53 + 60, 34 + 53 + 63, 34 + 53 + 98, 34 + 53 + 101, 34 + 53 +
103, 34 + 53 + 105, 34 + 53 + 106, 34 + 53 + 107, 34 + 54 + 56, 34 + 54 + 58, 34 + 54 + 59,
34 + 54 + 60, 34 + 54 + 63, 34 + 54 + 98, 34 + 54 + 101, 34 + 54 + 103, 34 + 54 + 105, 34 +
54 + 106, 34 + 54 + 107, 34 + 56 + 58, 34 + 56 + 59, 34 + 56 + 60, 34 + 56 + 63, 34 + 56 +
98, 34 + 56 + 101, 34 + 56 + 103, 34 + 56 + 105, 34 + 56 + 106, 34 + 56 + 107, 34 + 58 + 59,
34 + 58 + 60, 34 + 58 + 63, 34 + 58 + 98, 34 + 58 + 101, 34 + 58 + 103, 34 + 58 + 105, 34 +
58 + 106, 34 + 58 + 107, 34 + 59 + 60, 34 + 59 + 63, 34 + 59 + 98, 34 + 59 + 101, 34 + 59 +
103, 34 + 59 + 105, 34 + 59 + 106, 34 + 59 + 107, 34 + 60 + 63, 34 + 60 + 98, 34 + 60 + 101,
34 + 60 + 103, 34 + 60 + 105, 34 + 60 + 106, 34 + 60 + 107, 34 + 63 + 98, 34 + 63 + 101, 34 +
63 + 103, 34 + 63 + 105, 34 + 63 + 106, 34 + 63 + 107, 34 + 98 + 101, 34 + 98 + 103, 34 +
98 + 105, 34 + 98 + 106, 34 + 98 + 107, 34 + 101 + 103, 34 + 101 + 105, 34 + 101 + 106, 34 +
101 + 107, 34 + 103 + 105, 34 + 103 + 106, 34 + 103 + 107, 34 + 105 + 106, 34 + 105 + 107,
34 + 106 + 107, 35 + 50 + 51, 35 + 50 + 53, 35 + 50 + 54, 35 + 50 + 56, 35 + 50 + 58, 35 + 50 +
59, 35 + 50 + 60, 35 + 50 + 63, 35 + 50 + 98, 35 + 50 + 101, 35 + 50 + 103, 35 + 50 + 105,
35 + 50 + 106, 35 + 50 + 107, 35 + 51 + 53, 35 + 51 + 54, 35 + 51 + 56, 35 + 51 + 58, 35 + 51 +
59, 35 + 51 + 60, 35 + 51 + 63, 35 + 51 + 98, 35 + 51 + 101, 35 + 51 + 103, 35 + 51 + 105,
35 + 51 + 106, 35 + 51 + 107, 35 + 53 + 54, 35 + 53 + 56, 35 + 53 + 58, 35 + 53 + 59, 35 + 53 +
60, 35 + 53 + 63, 35 + 53 + 98, 35 + 53 + 101, 35 + 53 + 103, 35 + 53 + 105, 35 + 53 + 106,
35 + 53 + 107, 35 + 54 + 56, 35 + 54 + 58, 35 + 54 + 59, 35 + 54 + 60, 35 + 54 + 63, 35 + 54 +
98, 35 + 54 + 101, 35 + 54 + 103, 35 + 54 + 105, 35 + 54 + 106, 35 + 54 + 107, 35 + 56 +
58, 35 + 56 + 59, 35 + 56 + 60, 35 + 56 + 63, 35 + 56 + 98, 35 + 56 + 101, 35 + 56 + 103, 35 +
56 + 105, 35 + 56 + 106, 35 + 56 + 107, 35 + 58 + 59, 35 + 58 + 60, 35 + 58 + 63, 35 + 58 +
98, 35 + 58 + 101, 35 + 58 + 103, 35 + 58 + 105, 35 + 58 + 106, 35 + 58 + 107, 35 + 59 + 60,
35 + 59 + 63, 35 + 59 + 98, 35 + 59 + 101, 35 + 59 + 103, 35 + 59 + 105, 35 + 59 + 106, 35 +
59 + 107, 35 + 60 + 63, 35 + 60 + 98, 35 + 60 + 101, 35 + 60 + 103, 35 + 60 + 105, 35 + 60 +
106, 35 + 60 + 107, 35 + 63 + 98, 35 + 63 + 101, 35 + 63 + 103, 35 + 63 + 105, 35 + 63 + 106,
35 + 63 + 107, 35 + 98 + 101, 35 + 98 + 103, 35 + 98 + 105, 35 + 98 + 106, 35 + 98 + 107, 35 +
101 + 103, 35 + 101 + 105, 35 + 101 + 106, 35 + 101 + 107, 35 + 103 + 105, 35 + 103 +
106, 35 + 103 + 107, 35 + 105 + 106, 35 + 105 + 107, 35 + 106 + 107, 50 + 51 + 53, 50 + 51 +
54, 50 + 51 + 56, 50 + 51 + 58, 50 + 51 + 59, 50 + 51 + 60, 50 + 51 + 63, 50 + 51 + 98, 50 +
51 + 101, 50 + 51 + 103, 50 + 51 + 105, 50 + 51 + 106, 50 + 51 + 107, 50 + 53 + 54, 50 + 53 +
56, 50 + 53 + 58, 50 + 53 + 59, 50 + 53 + 60, 50 + 53 + 63, 50 + 53 + 98, 50 + 53 + 101, 50 +
53 + 103, 50 + 53 + 105, 50 + 53 + 106, 50 + 53 + 107, 50 + 54 + 56, 50 + 54 + 58, 50 + 54 +
59, 50 + 54 + 60, 50 + 54 + 63, 50 + 54 + 98, 50 + 54 + 101, 50 + 54 + 103, 50 + 54 + 105,
50 + 54 + 106, 50 + 54 + 107, 50 + 56 + 58, 50 + 56 + 59, 50 + 56 + 60, 50 + 56 + 63, 50 + 56 +
98, 50 + 56 + 101, 50 + 56 + 103, 50 + 56 + 105, 50 + 56 + 106, 50 + 56 + 107, 50 + 58 +
59, 50 + 58 + 60, 50 + 58 + 63, 50 + 58 + 98, 50 + 58 + 101, 50 + 58 + 103, 50 + 58 + 105, 50 +
58 + 106, 50 + 58 + 107, 50 + 59 + 60, 50 + 59 + 63, 50 + 59 + 98, 50 + 59 + 101, 50 + 59 +
103, 50 + 59 + 105, 50 + 59 + 106, 50 + 59 + 107, 50 + 60 + 63, 50 + 60 + 98, 50 + 60 + 101,
50 + 60 + 103, 50 + 60 + 105, 50 + 60 + 106, 50 + 60 + 107, 50 + 63 + 98, 50 + 63 + 101, 50 +
63 + 103, 50 + 63 + 105, 50 + 63 + 106, 50 + 63 + 107, 50 + 98 + 101, 50 + 98 + 103, 50 +
98 + 105, 50 + 98 + 106, 50 + 98 + 107, 50 + 101 + 103, 50 + 101 + 105, 50 + 101 + 106, 50 +
101 + 107, 50 + 103 + 105, 50 + 103 + 106, 50 + 103 + 107, 50 + 105 + 106, 50 + 105 + 107,
50 + 106 + 107, 51 + 53 + 54, 51 + 53 + 56, 51 + 53 + 58, 51 + 53 + 59, 51 + 53 + 60, 51 + 53 +
63, 51 + 53 + 98, 51 + 53 + 101, 51 + 53 + 103, 51 + 53 + 105, 51 + 53 + 106, 51 + 53 +
107, 51 + 54 + 56, 51 + 54 + 58, 51 + 54 + 59, 51 + 54 + 60, 51 + 54 + 63, 51 + 54 + 98, 51 +
54 + 101, 51 + 54 + 103, 51 + 54 + 105, 51 + 54 + 106, 51 + 54 + 107, 51 + 56 + 58, 51 + 56 +
59, 51 + 56 + 60, 51 + 56 + 63, 51 + 56 + 98, 51 + 56 + 101, 51 + 56 + 103, 51 + 56 + 105,
51 + 56 + 106, 51 + 56 + 107, 51 + 58 + 59, 51 + 58 + 60, 51 + 58 + 63, 51 + 58 + 98, 51 + 58 +
101, 51 + 58 + 103, 51 + 58 + 105, 51 + 58 + 106, 51 + 58 + 107, 51 + 59 + 60, 51 + 59 +
63, 51 + 59 + 98, 51 + 59 + 101, 51 + 59 + 103, 51 + 59 + 105, 51 + 59 + 106, 51 + 59 + 107,
51 + 60 + 63, 51 + 60 + 98, 51 + 60 + 101, 51 + 60 + 103, 51 + 60 + 105, 51 + 60 + 106, 51 +
60 + 107, 51 + 63 + 98, 51 + 63 + 101, 51 + 63 + 103, 51 + 63 + 105, 51 + 63 + 106, 51 + 63 +
107, 51 + 98 + 101, 51 + 98 + 103, 51 + 98 + 105, 51 + 98 + 106, 51 + 98 + 107, 51 + 101 +
103, 51 + 101 + 105, 51 + 101 + 106, 51 + 101 + 107, 51 + 103 + 105, 51 + 103 + 106, 51 +
103 + 107, 51 + 105 + 106, 51 + 105 + 107, 51 + 106 + 107, 53 + 54 + 56, 53 + 54 + 58, 53 +
54 + 59, 53 + 54 + 60, 53 + 54 + 63, 53 + 54 + 98, 53 + 54 + 101, 53 + 54 + 103, 53 + 54 +
105, 53 + 54 + 106, 53 + 54 + 107, 53 + 56 + 58, 53 + 56 + 59, 53 + 56 + 60, 53 + 56 + 63, 53 +
56 + 98, 53 + 56 + 101, 53 + 56 + 103, 53 + 56 + 105, 53 + 56 + 106, 53 + 56 + 107, 53 +
58 + 59, 53 + 58 + 60, 53 + 58 + 63, 53 + 58 + 98, 53 + 58 + 101, 53 + 58 + 103, 53 + 58 +
105, 53 + 58 + 106, 53 + 58 + 107, 53 + 59 + 60, 53 + 59 + 63, 53 + 59 + 98, 53 + 59 + 101,
53 + 59 + 103, 53 + 59 + 105, 53 + 59 + 106, 53 + 59 + 107, 53 + 60 + 63, 53 + 60 + 98, 53 +
60 + 101, 53 + 60 + 103, 53 + 60 + 105, 53 + 60 + 106, 53 + 60 + 107, 53 + 63 + 98, 53 + 63 +
101, 53 + 63 + 103, 53 + 63 + 105, 53 + 63 + 106, 53 + 63 + 107, 53 + 98 + 101, 53 + 98 +
103, 53 + 98 + 105, 53 + 98 + 106, 53 + 98 + 107, 53 + 101 + 103, 53 + 101 + 105, 53 + 101 +
106, 53 + 101 + 107, 53 + 103 + 105, 53 + 103 + 106, 53 + 103 + 107, 53 + 105 + 106, 53 +
105 + 107, 53 + 106 + 107, 54 + 56 + 58, 54 + 56 + 59, 54 + 56 + 60, 54 + 56 + 63, 54 + 56 +
98, 54 + 56 + 101, 54 + 56 + 103, 54 + 56 + 105, 54 + 56 + 106, 54 + 56 + 107, 54 + 58 + 59,
54 + 58 + 60, 54 + 58 + 63, 54 + 58 + 98, 54 + 58 + 101, 54 + 58 + 103, 54 + 58 + 105, 54 +
58 + 106, 54 + 58 + 107, 54 + 59 + 60, 54 + 59 + 63, 54 + 59 + 98, 54 + 59 + 101, 54 + 59 +
103, 54 + 59 + 105, 54 + 59 + 106, 54 + 59 + 107, 54 + 60 + 63, 54 + 60 + 98, 54 + 60 + 101,
54 + 60 + 103, 54 + 60 + 105, 54 + 60 + 106, 54 + 60 + 107, 54 + 63 + 98, 54 + 63 + 101, 54 +
63 + 103, 54 + 63 + 105, 54 + 63 + 106, 54 + 63 + 107, 54 + 98 + 101, 54 + 98 + 103, 54 +
98 + 105, 54 + 98 + 106, 54 + 98 + 107, 54 + 101 + 103, 54 + 101 + 105, 54 + 101 + 106, 54 +
101 + 107, 54 + 103 + 105, 54 + 103 + 106, 54 + 103 + 107, 54 + 105 + 106, 54 + 105 + 107,
54 + 106 + 107, 56 + 58 + 59, 56 + 58 + 60, 56 + 58 + 63, 56 + 58 + 98, 56 + 58 + 101, 56 +
58 + 103, 56 + 58 + 105, 56 + 58 + 106, 56 + 58 + 107, 56 + 59 + 60, 56 + 59 + 63, 56 + 59 +
98, 56 + 59 + 101, 56 + 59 + 103, 56 + 59 + 105, 56 + 59 + 106, 56 + 59 + 107, 56 + 60 + 63,
56 + 60 + 98, 56 + 60 + 101, 56 + 60 + 103, 56 + 60 + 105, 56 + 60 + 106, 56 + 60 + 107, 56 +
63 + 98, 56 + 63 + 101, 56 + 63 + 103, 56 + 63 + 105, 56 + 63 + 106, 56 + 63 + 107, 56 +
98 + 101, 56 + 98 + 103, 56 + 98 + 105, 56 + 98 + 106, 56 + 98 + 107, 56 + 101 + 103, 56 +
101 + 105, 56 + 101 + 106, 56 + 101 + 107, 56 + 103 + 105, 56 + 103 + 106, 56 + 103 + 107,
56 + 105 + 106, 56 + 105 + 107, 56 + 106 + 107, 58 + 59 + 60, 58 + 59 + 63, 58 + 59 + 98, 58 +
59 + 101, 58 + 59 + 103, 58 + 59 + 105, 58 + 59 + 106, 58 + 59 + 107, 58 + 60 + 63, 58 +
60 + 98, 58 + 60 + 101, 58 + 60 + 103, 58 + 60 + 105, 58 + 60 + 106, 58 + 60 + 107, 58 + 63 +
98, 58 + 63 + 101, 58 + 63 + 103, 58 + 63 + 105, 58 + 63 + 106, 58 + 63 + 107, 58 + 98 +
101, 58 + 98 + 103, 58 + 98 + 105, 58 + 98 + 106, 58 + 98 + 107, 58 + 101 + 103, 58 + 101 +
105, 58 + 101 + 106, 58 + 101 + 107, 58 + 103 + 105, 58 + 103 + 106, 58 + 103 + 107, 58 +
105 + 106, 58 + 105 + 107, 58 + 106 + 107, 59 + 60 + 63, 59 + 60 + 98, 59 + 60 + 101, 59 +
60 + 103, 59 + 60 + 105, 59 + 60 + 106, 59 + 60 + 107, 59 + 63 + 98, 59 + 63 + 101, 59 + 63 +
103, 59 + 63 + 105, 59 + 63 + 106, 59 + 63 + 107, 59 + 98 + 101, 59 + 98 + 103, 59 + 98 +
105, 59 + 98 + 106, 59 + 98 + 107, 59 + 101 + 103, 59 + 101 + 105, 59 + 101 + 106, 59 + 101 +
107, 59 + 103 + 105, 59 + 103 + 106, 59 + 103 + 107, 59 + 105 + 106, 59 + 105 + 107, 59 +
106 + 107, 60 + 63 + 98, 60 + 63 + 101, 60 + 63 + 103, 60 + 63 + 105, 60 + 63 + 106, 60 + 63 +
107, 60 + 98 + 101, 60 + 98 + 103, 60 + 98 + 105, 60 + 98 + 106, 60 + 98 + 107, 60 + 101 +
103, 60 + 101 + 105, 60 + 101 + 106, 60 + 101 + 107, 60 + 103 + 105, 60 + 103 + 106, 60 +
103 + 107, 60 + 105 + 106, 60 + 105 + 107, 60 + 106 + 107, 63 + 98 + 101, 63 + 98 + 103, 63 +
98 + 105, 63 + 98 + 106, 63 + 98 + 107, 63 + 101 + 103, 63 + 101 + 105, 63 + 101 + 106, 63 +
101 + 107, 63 + 103 + 105, 63 + 103 + 106, 63 + 103 + 107, 63 + 105 + 106, 63 + 105 +
107, 63 + 106 + 107, 98 + 101 + 103, 98 + 101 + 105, 98 + 101 + 106, 98 + 101 + 107, 98 +
103 + 105, 98 + 103 + 106, 98 + 103 + 107, 98 + 105 + 106, 98 + 105 + 107, 98 + 106 + 107,
101 + 103 + 105, 101 + 103 + 106, 101 + 103 + 107, 101 + 105 + 106, 101 + 105 + 107, 101 +
106 + 107, 103 + 105 + 106, 103 + 105 + 107, 103 + 106 + 107, 105 + 106 + 107

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 471, where the light chain variable domain includes a histidine at any of the specific combinations of one or more amino acid positions in SEQ ID NO: 471 listed in Table 12.

TABLE 12
Exemplary Combinations of Amino Acid Positions in SEQ
ID NO: 471 that can be substituted with histidine

29 + 32, 29 + 33, 29 + 34, 29 + 50, 29 + 51, 29 + 53, 29 + 55, 29 + 89, 29 + 92, 29 + 94, 29 +
96, 29 + 97, 32 + 33, 32 + 34, 32 + 50, 32 + 51, 32 + 53, 32 + 55, 32 + 89, 32 + 92, 32 + 94,
32 + 96, 32 + 97, 33 + 34, 33 + 50, 33 + 51, 33 + 53, 33 + 55, 33 + 89, 33 + 92, 33 + 94, 33 +
96, 33 + 97, 34 + 50, 34 + 51, 34 + 53, 34 + 55, 34 + 89, 34 + 92, 34 + 94, 34 + 96, 34 + 97,
50 + 51, 50 + 53, 50 + 55, 50 + 89, 50 + 92, 50 + 94, 50 + 96, 50 + 97, 51 + 53, 51 + 55, 51 +
89, 51 + 92, 51 + 94, 51 + 96, 51 + 97, 53 + 55, 53 + 89, 53 + 92, 53 + 94, 53 + 96, 53 + 97,
55 + 89, 55 + 92, 55 + 94, 55 + 96, 55 + 97, 89 + 92, 89 + 94, 89 + 96, 89 + 97, 92 + 94, 92 +
96, 92 + 97, 94 + 96, 94 + 97, 96 + 97, 29 + 32 + 33, 29 + 32 + 34, 29 + 32 + 50, 29 + 32 +
51, 29 + 32 + 53, 29 + 32 + 55, 29 + 32 + 89, 29 + 32 + 92, 29 + 32 + 94, 29 + 32 + 96, 29 +
32 + 97, 29 + 33 + 34, 29 + 33 + 50, 29 + 33 + 51, 29 + 33 + 53, 29 + 33 + 55, 29 + 33 +
89, 29 + 33 + 92, 29 + 33 + 94, 29 + 33 + 96, 29 + 33 + 97, 29 + 34 + 50, 29 + 34 + 51, 29 +
34 + 53, 29 + 34 + 55, 29 + 34 + 89, 29 + 34 + 92, 29 + 34 + 94, 29 + 34 + 96, 29 + 34 +
97, 29 + 50 + 51, 29 + 50 + 53, 29 + 50 + 55, 29 + 50 + 89, 29 + 50 + 92, 29 + 50 + 94, 29 +
50 + 96, 29 + 50 + 97, 29 + 51 + 53, 29 + 51 + 55, 29 + 51 + 89, 29 + 51 + 92, 29 + 51 +
94, 29 + 51 + 96, 29 + 51 + 97, 29 + 53 + 55, 29 + 53 + 89, 29 + 53 + 92, 29 + 53 + 94, 29 +
53 + 96, 29 + 53 + 97, 29 + 55 + 89, 29 + 55 + 92, 29 + 55 + 94, 29 + 55 + 96, 29 + 55 +
97, 29 + 89 + 92, 29 + 89 + 94, 29 + 89 + 96, 29 + 89 + 97, 29 + 92 + 94, 29 + 92 + 96, 29 +
92 + 97, 29 + 94 + 96, 29 + 94 + 97, 29 + 96 + 97, 32 + 33 + 34, 32 + 33 + 50, 32 + 33 +
51, 32 + 33 + 53, 32 + 33 + 55, 32 + 33 + 89, 32 + 33 + 92, 32 + 33 + 94, 32 + 33 + 96, 32 +
33 + 97, 32 + 34 + 50, 32 + 34 + 51, 32 + 34 + 53, 32 + 34 + 55, 32 + 34 + 89, 32 + 34 +
92, 32 + 34 + 94, 32 + 34 + 96, 32 + 34 + 97, 32 + 50 + 51, 32 + 50 + 53, 32 + 50 + 55, 32 +
50 + 89, 32 + 50 + 92, 32 + 50 + 94, 32 + 50 + 96, 32 + 50 + 97, 32 + 51 + 53, 32 + 51 +
55, 32 + 51 + 89, 32 + 51 + 92, 32 + 51 + 94, 32 + 51 + 96, 32 + 51 + 97, 32 + 53 + 55, 32 +
53 + 89, 32 + 53 + 92, 32 + 53 + 94, 32 + 53 + 96, 32 + 53 + 97, 32 + 55 + 89, 32 + 55 +
92, 32 + 55 + 94, 32 + 55 + 96, 32 + 55 + 97, 32 + 89 + 92, 32 + 89 + 94, 32 + 89 + 96, 32 +
89 + 97, 32 + 92 + 94, 32 + 92 + 96, 32 + 92 + 97, 32 + 94 + 96, 32 + 94 + 97, 32 + 96 +
97, 33 + 34 + 50, 33 + 34 + 51, 33 + 34 + 53, 33 + 34 + 55, 33 + 34 + 89, 33 + 34 + 92, 33 +
34 + 94, 33 + 34 + 96, 33 + 34 + 97, 33 + 50 + 51, 33 + 50 + 53, 33 + 50 + 55, 33 + 50 +
89, 33 + 50 + 92, 33 + 50 + 94, 33 + 50 + 96, 33 + 50 + 97, 33 + 51 + 53, 33 + 51 + 55, 33 +
51 + 89, 33 + 51 + 92, 33 + 51 + 94, 33 + 51 + 96, 33 + 51 + 97, 33 + 53 + 55, 33 + 53 +
89, 33 + 53 + 92, 33 + 53 + 94, 33 + 53 + 96, 33 + 53 + 97, 33 + 55 + 89, 33 + 55 + 92, 33 +
55 + 94, 33 + 55 + 96, 33 + 55 + 97, 33 + 89 + 92, 33 + 89 + 94, 33 + 89 + 96, 33 + 89 +
97, 33 + 92 + 94, 33 + 92 + 96, 33 + 92 + 97, 33 + 94 + 96, 33 + 94 + 97, 33 + 96 + 97, 34 +
50 + 51, 34 + 50 + 53, 34 + 50 + 55, 34 + 50 + 89, 34 + 50 + 92, 34 + 50 + 94, 34 + 50 +
96, 34 + 50 + 97, 34 + 51 + 53, 34 + 51 + 55, 34 + 51 + 89, 34 + 51 + 92, 34 + 51 + 94, 34 +
51 + 96, 34 + 51 + 97, 34 + 53 + 55, 34 + 53 + 89, 34 + 53 + 92, 34 + 53 + 94, 34 + 53 +
96, 34 + 53 + 97, 34 + 55 + 89, 34 + 55 + 92, 34 + 55 + 94, 34 + 55 + 96, 34 + 55 + 97, 34 +
89 + 92, 34 + 89 + 94, 34 + 89 + 96, 34 + 89 + 97, 34 + 92 + 94, 34 + 92 + 96, 34 + 92 +
97, 34 + 94 + 96, 34 + 94 + 97, 34 + 96 + 97, 50 + 51 + 53, 50 + 51 + 55, 50 + 51 + 89, 50 +
51 + 92, 50 + 51 + 94, 50 + 51 + 96, 50 + 51 + 97, 50 + 53 + 55, 50 + 53 + 89, 50 + 53 +
92, 50 + 53 + 94, 50 + 53 + 96, 50 + 53 + 97, 50 + 55 + 89, 50 + 55 + 92, 50 + 55 + 94, 50 +
55 + 96, 50 + 55 + 97, 50 + 89 + 92, 50 + 89 + 94, 50 + 89 + 96, 50 + 89 + 97, 50 + 92 +
94, 50 + 92 + 96, 50 + 92 + 97, 50 + 94 + 96, 50 + 94 + 97, 50 + 96 + 97, 51 + 53 + 55, 51 +
53 + 89, 51 + 53 + 92, 51 + 53 + 94, 51 + 53 + 96, 51 + 53 + 97, 51 + 55 + 89, 51 + 55 +
92, 51 + 55 + 94, 51 + 55 + 96, 51 + 55 + 97, 51 + 89 + 92, 51 + 89 + 94, 51 + 89 + 96, 51 +
89 + 97, 51 + 92 + 94, 51 + 92 + 96, 51 + 92 + 97, 51 + 94 + 96, 51 + 94 + 97, 51 + 96 +
97, 53 + 55 + 89, 53 + 55 + 92, 53 + 55 + 94, 53 + 55 + 96, 53 + 55 + 97, 53 + 89 + 92, 53 +
89 + 94, 53 + 89 + 96, 53 + 89 + 97, 53 + 92 + 94, 53 + 92 + 96, 53 + 92 + 97, 53 + 94 +
96, 53 + 94 + 97, 53 + 96 + 97, 55 + 89 + 92, 55 + 89 + 94, 55 + 89 + 96, 55 + 89 + 97, 55 +
92 + 94, 55 + 92 + 96, 55 + 92 + 97, 55 + 94 + 96, 55 + 94 + 97, 55 + 96 + 97, 89 + 92 +
94, 89 + 92 + 96, 89 + 92 + 97, 89 + 94 + 96, 89 + 94 + 97, 89 + 96 + 97, 92 + 94 + 96, 92 +
94 + 97, 92 + 96 + 97, 94 + 96 + 97

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11; and a light chain variable domain that that is at least 90% (e.g., at least 92%, at least 94%, at least 96%, at least 98%, at least 99%, or 100%) identical to SEQ ID NO: 471, where the light chain variable domain includes a histidine at any of the specific combinations of one or more amino acid positions in SEQ ID NO: 471 listed in Table 12.

In some examples of any of the ABPCs described herein, the first antigen-binding domain comprises a light chain variable domain that is at least 90% identical to SEQ ID NO: 471, wherein the light chain variable domain includes a histidine at position 29 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain comprising SEQ ID NO: 471, and a heavy chain variable domain that is at least 90% identical (e.g., at least 92%, at least 94%, at least 96%, at least 98%, at least 99%, or 100%) identical to SEQ ID NO: 470, where the heavy chain variable domain includes a histidine at any of the specific combinations of one or more (e.g., two, three, four, five, six, seven, eight, nine, or ten) amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 32 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 33 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 34 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 50 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 51 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 53 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 55 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 89 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 92 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 94 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 96 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain comprises a light chain variable domain that is at least 90% identical to SEQ ID NO: 471, wherein the light chain variable domain includes a histidine at position 97 in SEQ ID NO: 471; and a heavy chain variable domain that is at least 90% (e.g., at least 92%, at least 94%, at least 96%, at least 98%, at least 99%, or 100%) identical to SEQ ID NO: 470, where the heavy chain variable domain includes a histidine at any of the specific combinations of one or more amino acid positions in SEQ ID NO: 470 listed in Table 11.

In some examples of any of the ABPCs described herein, the first antigen-binding domain includes a heavy chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain SC16.34 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.34 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.34 comprises SEQ ID NO: 470. In some examples of any of the ABPCs described herein, the light chain variable domain of SC16.34 comprises SEQ ID NO: 471.

In some examples of any of the ABPCs described herein, the first antigen-binding domain comprises a heavy chain variable domain comprising a CDR1, a CDR2, and a CDR3 of SEQ ID NO: 646, SEQ ID NO: 647, and SEQ ID NO: 648, respectively, with collectively a total of one or more (e.g., one, two, three, four, five, six, seven, eight, nine, or ten) histidine(s) in SEQ ID NOs: 646-648 substituted with an alanine. In some examples of any of the ABPCs described herein, the first antigen-binding domain comprises a light chain variable domain comprising a CDR1, a CDR2, and a CDR3 of SEQ ID NO: 649, SEQ ID NO: 650, and SEQ ID NO: 651, respectively, with collectively a total of one or more (e.g., one, two, three, four, five, six, seven, eight, nine, or ten) histidine(s) in SEQ ID NOs: 649-651 substituted with an alanine. In some examples of any of the ABPCs described herein, the first antigen-binding domain includes: a heavy chain variable domain comprising a CDR1, a CDR2, and a CDR3 of SEQ ID NO: 646, SEQ ID NO: 647, and SEQ ID NO: 648, respectively, with collectively a total of one or more (e.g., one, two, three, four, five, six, seven, eight, nine, or ten) histidine(s) in SEQ ID NOs: 646-648 substituted with an alanine; and a light chain variable domain comprising a CDR1, a CDR2, and a CDR3 of SEQ ID NO: 649, SEQ ID NO: 650, and SEQ ID NO: 651, respectively, with collectively a total of one or more (e.g., one, two, three, four, five, six, seven, eight, nine, or ten) histidine(s) in SEQ ID NOs: 649-651 substituted with an alanine.

In some examples of any of the ABPCs described herein, the first antigen-binding domain comprises a heavy chain variable domain of SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain of SEQ ID NO: 471, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, or SEQ ID NO: 536.

In some examples of any of the ABPCs described herein, the first antigen-binding domain includes a light chain variable domain comprising SEQ ID NO: 471, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 513, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 514, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 515, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 516, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 517, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 518, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 519, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 520, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 521, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 522, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 523, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 524, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 525, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 526, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 527, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 528, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 529, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 530, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 531, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 532, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 533, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 534, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 535, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a light chain variable domain comprising SEQ ID NO: 536, and a heavy chain variable domain comprising SEQ ID NO: 470, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, or SEQ ID NO: 512.

In some examples of any of the ABPCs described herein, the first antigen-binding domain includes a heavy chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a light chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the first antigen-binding domain includes a heavy chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine; and a light chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) amino acids substituted with a histidine. In some examples of any of the ABPCs described herein, the heavy chain variable domain of SC16.67 comprises SEQ ID NO: 537. In some examples of any of the ABPCs described herein, the light chain variable domain of SC16.67 comprises SEQ ID NO: 538.

In some examples of any of the ABPCs described herein, the first antigen-binding domain comprises a heavy chain variable domain comprising a CDR1, a CDR2, and a CDR3 of SEQ ID NO: 652, SEQ ID NO: 653, and SEQ ID NO: 654, respectively, with collectively a total of one or more (e.g., one, two, three, four, five, six, seven, eight, nine, or ten) amino acid positions in SEQ ID NOs: 652-654 substituted with a histidine. In some examples of any of the ABPCs described herein, the first antigen-binding domain comprises a light chain variable domain comprising a CDR1, a CDR2, and a CDR3 of SEQ ID NO: 655 SEQ ID NO: 656, and SEQ ID NO: 657, respectively, with collectively a total of one or more (e.g., one, two, three, four, five, six, seven, eight, nine, or ten) amino acid positions in SEQ ID NOs: 655-657 substituted with a histidine. In some examples of any of the ABPCs described herein, the first antigen-binding domain includes: a heavy chain variable domain comprising a CDR1, a CDR2, and a CDR3 of SEQ ID NO: 652, SEQ ID NO: 653, and SEQ ID NO: 654, respectively, with collectively a total of one or more (e.g., one, two, three, four, five, six, seven, eight, nine, or ten) amino acid positions in SEQ ID NOs: 652-654 substituted with a histidine; and a light chain variable domain comprising a CDR1, a CDR2, and a CDR3 of SEQ ID NO: 655, SEQ ID NO: 656, and SEQ ID NO: 657, respectively, with collectively a total of one or more (e.g., one, two, three, four, five, six, seven, eight, nine, or ten) amino acid positions in SEQ ID NOs: 655-657 substituted with a histidine.

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 537, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 537 selected from the group consisting of: 26, 29, 31, 32, 53, 54, 57, 58, 59, 65, 67, 68, 103, 104, and 106. In some examples of any of the ABPCs described herein, the first antigen-binding domain includes a light chain variable domain that is at least 90% (e.g., at least 92%, at least 94%, at least 96%, at least 98%, at least 99%, or 100%) identical to SEQ ID NO: 538, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 538 selected from the group consisting of: 28, 34, 53, 93, 94, and 98. In some examples of any of the ABPCs described herein the first antigen-binding domain includes: a heavy chain variable domain that is at least 90% (e.g., at least 92%, at least 94%, at least 96%, at least 98%, at least 99%, or 100%) identical to SEQ ID NO: 537, where the heavy chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 537 selected from the group consisting of: 26, 29, 31, 32, 53, 54, 57, 58, 59, 65, 67, 68, 103, 104, and 106 and a light chain variable domain that is at least 90% (e.g., at least 92%, at least 94%, at least 96%, at least 98%, at least 99%, or 100%) identical to SEQ ID NO: 538, where the light chain variable domain includes a histidine at one or more (e.g., one, two, three, four, five, six, seven, eight, nine, ten, eleven, twelve, thirteen, fourteen, fifteen, sixteen, seventeen, eighteen, nineteen, or twenty) amino acid positions in SEQ ID NO: 538 selected from the group consisting of: 28, 34, 53, 93, 94, and 98.

In some examples of any of the ABPCs described herein, a heavy chain variable domain includes a heavy chain variable domain that is at least 90% (e.g., at least 92%, at least 94%, at least 96%, at least 98%, at least 99%, or 100%) identical to SEQ ID NO: 537, where the heavy chain variable domain includes a histidine at any of the specific combinations of one or more amino acid positions in SEQ ID NO: 537 listed in Table 13.

TABLE 13
Exemplary Combinations of Amino Acid Positions in SEQ
ID NO: 537 that can be substituted with histidine

26 + 29, 26 + 31, 26 + 32, 26 + 53, 26 + 54, 26 + 57, 26 + 58, 26 + 59, 26 + 65, 26 + 67, 26 +
68, 26 + 103, 26 + 104, 26 + 106, 29 + 31, 29 + 32, 29 + 53, 29 + 54, 29 + 57, 29 + 58, 29 +
59, 29 + 65, 29 + 67, 29 + 68, 29 + 103, 29 + 104, 29 + 106, 31 + 32, 31 + 53, 31 + 54, 31 +
57, 31 + 58, 31 + 59, 31 + 65, 31 + 67, 31 + 68, 31 + 103, 31 + 104, 31 + 106, 32 + 53, 32 +
54, 32 + 57, 32 + 58, 32 + 59, 32 + 65, 32 + 67, 32 + 68, 32 + 103, 32 + 104, 32 + 106, 53 +
54, 53 + 57, 53 + 58, 53 + 59, 53 + 65, 53 + 67, 53 + 68, 53 + 103, 53 + 104, 53 + 106, 54 +
57, 54 + 58, 54 + 59, 54 + 65, 54 + 67, 54 + 68, 54 + 103, 54 + 104, 54 + 106, 57 + 58, 57 +
59, 57 + 65, 57 + 67, 57 + 68, 57 + 103, 57 + 104, 57 + 106, 58 + 59, 58 + 65, 58 + 67, 58 +
68, 58 + 103, 58 + 104, 58 + 106, 59 + 65, 59 + 67, 59 + 68, 59 + 103, 59 + 104, 59 + 106, 65 +
67, 65 + 68, 65 + 103, 65 + 104, 65 + 106, 67 + 68, 67 + 103, 67 + 104, 67 + 106, 68 + 103,
68 + 104, 68 + 106, 103 + 104, 103 + 106, 104 + 106, 26 + 29 + 31, 26 + 29 + 32, 26 + 29 +
53, 26 + 29 + 54, 26 + 29 + 57, 26 + 29 + 58, 26 + 29 + 59, 26 + 29 + 65, 26 + 29 + 67, 26 +
29 + 68, 26 + 29 + 103, 26 + 29 + 104, 26 + 29 + 106, 26 + 31 + 32, 26 + 31 + 53, 26 + 31 +
54, 26 + 31 + 57, 26 + 31 + 58, 26 + 31 + 59, 26 + 31 + 65, 26 + 31 + 67, 26 + 31 + 68, 26 +
31 + 103, 26 + 31 + 104, 26 + 31 + 106, 26 + 32 + 53, 26 + 32 + 54, 26 + 32 + 57, 26 + 32 +
58, 26 + 32 + 59, 26 + 32 + 65, 26 + 32 + 67, 26 + 32 + 68, 26 + 32 + 103, 26 + 32 + 104, 26 +
32 + 106, 26 + 53 + 54, 26 + 53 + 57, 26 + 53 + 58, 26 + 53 + 59, 26 + 53 + 65, 26 + 53 +
67, 26 + 53 + 68, 26 + 53 + 103, 26 + 53 + 104, 26 + 53 + 106, 26 + 54 + 57, 26 + 54 + 58, 26 +
54 + 59, 26 + 54 + 65, 26 + 54 + 67, 26 + 54 + 68, 26 + 54 + 103, 26 + 54 + 104, 26 + 54 +
106, 26 + 57 + 58, 26 + 57 + 59, 26 + 57 + 65, 26 + 57 + 67, 26 + 57 + 68, 26 + 57 + 103, 26 +
57 + 104, 26 + 57 + 106, 26 + 58 + 59, 26 + 58 + 65, 26 + 58 + 67, 26 + 58 + 68, 26 + 58 +
103, 26 + 58 + 104, 26 + 58 + 106, 26 + 59 + 65, 26 + 59 + 67, 26 + 59 + 68, 26 + 59 + 103,
26 + 59 + 104, 26 + 59 + 106, 26 + 65 + 67, 26 + 65 + 68, 26 + 65 + 103, 26 + 65 + 104, 26 +
65 + 106, 26 + 67 + 68, 26 + 67 + 103, 26 + 67 + 104, 26 + 67 + 106, 26 + 68 + 103, 26 + 68 +
104, 26 + 68 + 106, 26 + 103 + 104, 26 + 103 + 106, 26 + 104 + 106, 29 + 31 + 32, 29 + 31 +
53, 29 + 31 + 54, 29 + 31 + 57, 29 + 31 + 58, 29 + 31 + 59, 29 + 31 + 65, 29 + 31 + 67, 29 +
31 + 68, 29 + 31 + 103, 29 + 31 + 104, 29 + 31 + 106, 29 + 32 + 53, 29 + 32 + 54, 29 + 32 +
57, 29 + 32 + 58, 29 + 32 + 59, 29 + 32 + 65, 29 + 32 + 67, 29 + 32 + 68, 29 + 32 + 103, 29 +
32 + 104, 29 + 32 + 106, 29 + 53 + 54, 29 + 53 + 57, 29 + 53 + 58, 29 + 53 + 59, 29 + 53 +
65, 29 + 53 + 67, 29 + 53 + 68, 29 + 53 + 103, 29 + 53 + 104, 29 + 53 + 106, 29 + 54 + 57, 29 +
54 + 58, 29 + 54 + 59, 29 + 54 + 65, 29 + 54 + 67, 29 + 54 + 68, 29 + 54 + 103, 29 + 54 +
104, 29 + 54 + 106, 29 + 57 + 58, 29 + 57 + 59, 29 + 57 + 65, 29 + 57 + 67, 29 + 57 + 68, 29 +
57 + 103, 29 + 57 + 104, 29 + 57 + 106, 29 + 58 + 59, 29 + 58 + 65, 29 + 58 + 67, 29 + 58 +
68, 29 + 58 + 103, 29 + 58 + 104, 29 + 58 + 106, 29 + 59 + 65, 29 + 59 + 67, 29 + 59 + 68, 29 +
59 + 103, 29 + 59 + 104, 29 + 59 + 106, 29 + 65 + 67, 29 + 65 + 68, 29 + 65 + 103, 29 + 65 +
104, 29 + 65 + 106, 29 + 67 + 68, 29 + 67 + 103, 29 + 67 + 104, 29 + 67 + 106, 29 + 68 +
103, 29 + 68 + 104, 29 + 68 + 106, 29 + 103 + 104, 29 + 103 + 106, 29 + 104 + 106, 31 + 32 +
53, 31 + 32 + 54, 31 + 32 + 57, 31 + 32 + 58, 31 + 32 + 59, 31 + 32 + 65, 31 + 32 + 67, 31 +
32 + 68, 31 + 32 + 103, 31 + 32 + 104, 31 + 32 + 106, 31 + 53 + 54, 31 + 53 + 57, 31 + 53 +
58, 31 + 53 + 59, 31 + 53 + 65, 31 + 53 + 67, 31 + 53 + 68, 31 + 53 + 103, 31 + 53 + 104, 31 +
53 + 106, 31 + 54 + 57, 31 + 54 + 58, 31 + 54 + 59, 31 + 54 + 65, 31 + 54 + 67, 31 + 54 +
68, 31 + 54 + 103, 31 + 54 + 104, 31 + 54 + 106, 31 + 57 + 58, 31 + 57 + 59, 31 + 57 + 65, 31 +
57 + 67, 31 + 57 + 68, 31 + 57 + 103, 31 + 57 + 104, 31 + 57 + 106, 31 + 58 + 59, 31 + 58 +
65, 31 + 58 + 67, 31 + 58 + 68, 31 + 58 + 103, 31 + 58 + 104, 31 + 58 + 106, 31 + 59 + 65, 31 +
59 + 67, 31 + 59 + 68, 31 + 59 + 103, 31 + 59 + 104, 31 + 59 + 106, 31 + 65 + 67, 31 + 65 +
68, 31 + 65 + 103, 31 + 65 + 104, 31 + 65 + 106, 31 + 67 + 68, 31 + 67 + 103, 31 + 67 + 104,
31 + 67 + 106, 31 + 68 + 103, 31 + 68 + 104, 31 + 68 + 106, 31 + 103 + 104, 31 + 103 + 106,
31 + 104 + 106, 32 + 53 + 54, 32 + 53 + 57, 32 + 53 + 58, 32 + 53 + 59, 32 + 53 + 65, 32 + 53 +
67, 32 + 53 + 68, 32 + 53 + 103, 32 + 53 + 104, 32 + 53 + 106, 32 + 54 + 57, 32 + 54 + 58,
32 + 54 + 59, 32 + 54 + 65, 32 + 54 + 67, 32 + 54 + 68, 32 + 54 + 103, 32 + 54 + 104, 32 + 54 +
106, 32 + 57 + 58, 32 + 57 + 59, 32 + 57 + 65, 32 + 57 + 67, 32 + 57 + 68, 32 + 57 + 103,
32 + 57 + 104, 32 + 57 + 106, 32 + 58 + 59, 32 + 58 + 65, 32 + 58 + 67, 32 + 58 + 68, 32 + 58 +
103, 32 + 58 + 104, 32 + 58 + 106, 32 + 59 + 65, 32 + 59 + 67, 32 + 59 + 68, 32 + 59 + 103,
32 + 59 + 104, 32 + 59 + 106, 32 + 65 + 67, 32 + 65 + 68, 32 + 65 + 103, 32 + 65 + 104, 32 +
65 + 106, 32 + 67 + 68, 32 + 67 + 103, 32 + 67 + 104, 32 + 67 + 106, 32 + 68 + 103, 32 + 68 +
104, 32 + 68 + 106, 32 + 103 + 104, 32 + 103 + 106, 32 + 104 + 106, 53 + 54 + 57, 53 + 54 +
58, 53 + 54 + 59, 53 + 54 + 65, 53 + 54 + 67, 53 + 54 + 68, 53 + 54 + 103, 53 + 54 + 104,
53 + 54 + 106, 53 + 57 + 58, 53 + 57 + 59, 53 + 57 + 65, 53 + 57 + 67, 53 + 57 + 68, 53 + 57 +
103, 53 + 57 + 104, 53 + 57 + 106, 53 + 58 + 59, 53 + 58 + 65, 53 + 58 + 67, 53 + 58 + 68,
53 + 58 + 103, 53 + 58 + 104, 53 + 58 + 106, 53 + 59 + 65, 53 + 59 + 67, 53 + 59 + 68, 53 +
59 + 103, 53 + 59 + 104, 53 + 59 + 106, 53 + 65 + 67, 53 + 65 + 68, 53 + 65 + 103, 53 + 65 +
104, 53 + 65 + 106, 53 + 67 + 68, 53 + 67 + 103, 53 + 67 + 104, 53 + 67 + 106, 53 + 68 + 103,
53 + 68 + 104, 53 + 68 + 106, 53 + 103 + 104, 53 + 103 + 106, 53 + 104 + 106, 54 + 57 + 58,
54 + 57 + 59, 54 + 57 + 65, 54 + 57 + 67, 54 + 57 + 68, 54 + 57 + 103, 54 + 57 + 104, 54 + 57 +
106, 54 + 58 + 59, 54 + 58 + 65, 54 + 58 + 67, 54 + 58 + 68, 54 + 58 + 103, 54 + 58 + 104,
54 + 58 + 106, 54 + 59 + 65, 54 + 59 + 67, 54 + 59 + 68, 54 + 59 + 103, 54 + 59 + 104, 54 +
59 + 106, 54 + 65 + 67, 54 + 65 + 68, 54 + 65 + 103, 54 + 65 + 104, 54 + 65 + 106, 54 + 67 +
68, 54 + 67 + 103, 54 + 67 + 104, 54 + 67 + 106, 54 + 68 + 103, 54 + 68 + 104, 54 + 68 + 106,
54 + 103 + 104, 54 + 103 + 106, 54 + 104 + 106, 57 + 58 + 59, 57 + 58 + 65, 57 + 58 + 67, 57 +
58 + 68, 57 + 58 + 103, 57 + 58 + 104, 57 + 58 + 106, 57 + 59 + 65, 57 + 59 + 67, 57 + 59 +
68, 57 + 59 + 103, 57 + 59 + 104, 57 + 59 + 106, 57 + 65 + 67, 57 + 65 + 68, 57 + 65 + 103,
57 + 65 + 104, 57 + 65 + 106, 57 + 67 + 68, 57 + 67 + 103, 57 + 67 + 104, 57 + 67 + 106, 57 +
68 + 103, 57 + 68 + 104, 57 + 68 + 106, 57 + 103 + 104, 57 + 103 + 106, 57 + 104 + 106, 58 +
59 + 65, 58 + 59 + 67, 58 + 59 + 68, 58 + 59 + 103, 58 + 59 + 104, 58 + 59 + 106, 58 + 65 +
67, 58 + 65 + 68, 58 + 65 + 103, 58 + 65 + 104, 58 + 65 + 106, 58 + 67 + 68, 58 + 67 + 103,
58 + 67 + 104, 58 + 67 + 106, 58 + 68 + 103, 58 + 68 + 104, 58 + 68 + 106, 58 + 103 + 104,
58 + 103 + 106, 58 + 104 + 106, 59 + 65 + 67, 59 + 65 + 68, 59 + 65 + 103, 59 + 65 + 104, 59 +
65 + 106, 59 + 67 + 68, 59 + 67 + 103, 59 + 67 + 104, 59 + 67 + 106, 59 + 68 + 103, 59 +
68 + 104, 59 + 68 + 106, 59 + 103 + 104, 59 + 103 + 106, 59 + 104 + 106, 65 + 67 + 68, 65 +
67 + 103, 65 + 67 + 104, 65 + 67 + 106, 65 + 68 + 103, 65 + 68 + 104, 65 + 68 + 106, 65 +
103 + 104, 65 + 103 + 106, 65 + 104 + 106, 67 + 68 + 103, 67 + 68 + 104, 67 + 68 + 106, 67 +
103 + 104, 67 + 103 + 106, 67 + 104 + 106, 68 + 103 + 104, 68 + 103 + 106, 68 + 104 + 106,
103 + 104 + 106

In some examples of any of the ABPCs described herein, a light chain variable domain includes a light chain variable domain that is at least 90% (e.g., at least 92%, at least 94%, at least 96%, at least 98%, at least 99%, or 100%) identical to SEQ ID NO: 538, where the light chain variable domain includes a histidine at any of the specific combinations of one or more amino acid positions in SEQ ID NO: 538 listed in Table 14.

TABLE 14
Exemplary Combinations of Amino Acid Positions in SEQ
ID NO: 538 that can be substituted with histidine

28 + 34, 28 + 53, 28 + 93, 28 + 94, 28 + 98, 34 + 53, 34 + 93, 34 + 94, 34 + 98, 53 + 93, 53 +
94, 53 + 98, 93 + 94, 93 + 98, 94 + 98, 28 + 34 + 53, 28 + 34 + 93, 28 + 34 + 94, 28 + 34 +
98, 28 + 53 + 93, 28 + 53 + 94, 28 + 53 + 98, 28 + 93 + 94, 28 + 93 + 98, 28 + 94 + 98, 34 +
53 + 93, 34 + 53 + 94, 34 + 53 + 98, 34 + 93 + 94, 34 + 93 + 98, 34 + 94 + 98, 53 + 93 + 94,
53 + 93 + 98, 53 + 94 + 98, 93 + 94 + 98

In some examples of any of the ABPCs described herein, the first antigen-binding domain includes a heavy chain variable domain that is at least 90% (e.g., at least 92%, at least 94%, at least 96%, at least 98%, at least 99%, or 100%) identical to SEQ ID NO: 537, where the heavy chain variable domain includes a histidine at any of the specific combinations of one or more amino acid positions in SEQ ID NO: 537 listed in Table 13; and a light chain variable domain that that is at least 90% (e.g., at least 92%, at least 94%, at least 96%, at least 98%, at least 99%, or 100%) identical to SEQ ID NO: 538, where the light chain variable domain includes a histidine at any of the specific combinations of one or more amino acid positions in SEQ ID NO: 538 listed in Table 14.

In some examples of any of the ABPCs described herein, the first antigen-binding domain comprises a light chain variable domain that is at least 90% identical to SEQ ID NO: 538, wherein the light chain variable domain includes a histidine at position 28 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain comprises a light chain variable domain comprising SEQ ID NO: 538, and a heavy chain variable domain that is at least 90% identical (e.g., at least 92%, at least 94%, at least 96%, at least 98%, at least 99%, or 100%) identical to SEQ ID NO: 538, where the heavy chain variable domain includes a histidine at any of the specific combinations of one or more (e.g., two, three, four, five, six, seven, eight, nine, or ten) amino acid positions in SEQ ID NO: 537 listed in Table 13.

In some examples of any of the ABPCs described herein, the first antigen-binding domain comprises a light chain variable domain that is at least 90% identical to SEQ ID NO: 538, wherein the light chain variable domain includes a histidine at position 34 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain comprises a light chain variable domain that is at least 90% identical to SEQ ID NO: 538, wherein the light chain variable domain includes a histidine at position 53 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain comprises a light chain variable domain that is at least 90% identical to SEQ ID NO: 538, wherein the light chain variable domain includes a histidine at position 93 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain comprises a light chain variable domain that is at least 90% identical to SEQ ID NO: 538, wherein the light chain variable domain includes a histidine at position 94 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain comprises a light chain variable domain that is at least 90% identical to SEQ ID NO: 538, wherein the light chain variable domain includes a histidine at position 98 in SEQ ID NO: 538; and a heavy chain variable domain that is at least 90% (e.g., at least 92%, at least 94%, at least 96%, at least 98%, at least 99%, or 100%) identical to SEQ ID NO: 538, where the heavy chain variable domain includes a histidine at any of the specific combinations of one or more amino acid positions in SEQ ID NO: 537 listed in Table 13.

In some examples of any of the ABPCs described herein, the first antigen-binding domain includes a heavy chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a light chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the first antigen-binding domain includes a heavy chain variable domain SC16.67 with one or more (e.g., one, two, three, four, five, six, seven, eight, nine, ten, eleven, twelve, thirteen, fourteen, fifteen, sixteen, seventeen, eighteen, nineteen, or twenty) histidines substituted with an alanine; and a light chain variable domain of SC16.67 with one or more (e.g., one, two, three, four, five, six, seven, eight, nine, ten, eleven, twelve, thirteen, fourteen, fifteen, sixteen, seventeen, eighteen, nineteen, or twenty) histidine(s) substituted with an alanine. In some examples of any of the ABPCs described herein, the heavy chain variable domain of SC16.67 comprises SEQ ID NO: 537. In some examples of any of the ABPCs described herein, the light chain variable domain of SC16.67 comprises SEQ ID NO: 538.

In some examples of any of the ABPCs described herein, the first antigen-binding domain comprises a heavy chain variable domain comprising a CDR1, a CDR2, and a CDR3 of SEQ ID NO: 652, SEQ ID NO: 653, and SEQ ID NO: 654, respectively, with collectively a total of one or more (e.g., one, two, three, four, five, six, seven, eight, nine, or ten) histidine(s) in SEQ ID NOs: 652-654 substituted with an alanine. In some examples of any of the ABPCs described herein, the first antigen-binding domain comprises a light chain variable domain comprising a CDR1, a CDR2, and a CDR3 of SEQ ID NO: 655, SEQ ID NO: 656, and SEQ ID NO: 657, respectively, with collectively a total of one or more (e.g., one, two, three, four, five, six, seven, eight, nine, or ten) histidine(s) in SEQ ID NOs: 655-657 substituted with an alanine. In some examples of any of the ABPCs described herein, the first antigen-binding domain includes: a heavy chain variable domain comprising a CDR1, a CDR2, and a CDR3 of SEQ ID NO: 652, SEQ ID NO: 653, and SEQ ID NO: 654, respectively, with collectively a total of one or more (e.g., one, two, three, four, five, six, seven, eight, nine, or ten) histidine(s) in SEQ ID NOs: 652-654 substituted with an alanine; and a light chain variable domain comprising a CDR1, a CDR2, and a CDR3 of SEQ ID NO: 655, SEQ ID NO: 656, and SEQ ID NO: 657, respectively, with collectively a total of one or more (e.g., one, two, three, four, five, six, seven, eight, nine, or ten) histidine(s) in SEQ ID NOs: 655-657 substituted with an alanine.

In some examples of any of the ABPCs described herein, the first antigen-binding domain comprises a heavy chain variable domain of SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain of SEQ ID NO: 538, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, or SEQ ID NO: 605.

In some examples of any of the ABPCs described herein, the first antigen-binding domain includes a light chain variable domain comprising SEQ ID NO: 538, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 576, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 577, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 578, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 579, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 580, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 581, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 582, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 583, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 584, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 585, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 586, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 587, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 588, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 589, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 590, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 591, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 592, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 593, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 594, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 595, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 596, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 597, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 598, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 599, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 600, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 601, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 602, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 603, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 604, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 605, and a heavy chain variable domain comprising SEQ ID NO: 537, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, or SEQ ID NO: 575.

In some examples of any of the ABPCs described herein, the first antigen-binding domain includes a light chain variable domain comprising SEQ ID NO: 581, and a heavy chain variable domain comprising SEQ ID NO: 553. In some examples of any of the ABPCs described herein, the first antigen-binding domain includes a light chain variable domain comprising SEQ ID NO: 581, and a heavy chain variable domain comprising SEQ ID NO: 557. In some examples of any of the ABPCs described herein, the first antigen-binding domain includes a light chain variable domain comprising SEQ ID NO: 581, and a heavy chain variable domain comprising SEQ ID NO: 558. In some examples of any of the ABPCs described herein, the first antigen-binding domain includes a light chain variable domain comprising SEQ ID NO: 587, and a heavy chain variable domain comprising SEQ ID NO: 553. In some examples of any of the ABPCs described herein, the first antigen-binding domain includes a light chain variable domain comprising SEQ ID NO: 587, and a heavy chain variable domain comprising SEQ ID NO: 557. In some examples of any of the ABPCs described herein, the first antigen-binding domain includes a light chain variable domain comprising SEQ ID NO: 587, and a heavy chain variable domain comprising SEQ ID NO: 558. In some examples of any of the ABPCs described herein, the first antigen-binding domain includes a light chain variable domain comprising SEQ ID NO: 599, and a heavy chain variable domain comprising SEQ ID NO: 553. In some examples of any of the ABPCs described herein, the first antigen-binding domain includes a light chain variable domain comprising SEQ ID NO: 599, and a heavy chain variable domain comprising SEQ ID NO: 557. In some examples of any of the ABPCs described herein, the first antigen-binding domain includes a light chain variable domain comprising SEQ ID NO: 599, and a heavy chain variable domain comprising SEQ ID NO: 558. In some examples of any of the ABPCs described herein, the first antigen-binding domain includes a light chain variable domain comprising SEQ ID NO: 600, and a heavy chain variable domain comprising SEQ ID NO: 553. In some examples of any of the ABPCs described herein, the first antigen-binding domain includes a light chain variable domain comprising SEQ ID NO: 600, and a heavy chain variable domain comprising SEQ ID NO: 557. In some examples of any of the ABPCs described herein, the first antigen-binding domain includes a light chain variable domain comprising SEQ ID NO: 600, and a heavy chain variable domain comprising SEQ ID NO: 558.

In some examples of any of the ABPCs described herein, the first antigen-binding domain can include a sequence that is at least 80% identical (e.g., at least 85% identical, at least 90% identical, at least 92% identical, at least 94% identical, at least 96% identical, at least 98% identical, at least 99% identical, or 100% identical) to any one of SEQ ID NOs: 606-620.

Also provided herein are pharmaceutical compositions including any of the ABPCs described herein. Also provided herein are methods of treating a subject in need thereof that include administering a therapeutically effective amount of any of the ABPCs described herein to the subject.

In some examples of any of the ABPCs described herein, a composition including the ABPC (e.g., any of the ABPCs described herein) can provide for an increase (e.g., a detectable increase) (e.g., at least a 1% increase, at least a 2% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 120% increase, at least a 140% increase, at least a 160% increase, at least a 180% increase, at least a 200% increase, at least a 250% increase, at least a 300% increase, at least a 350% increase, at least a 400% increase, at least a 450% increase, at least a 500% increase, at least a 1,000% increase, at least a 2,000% increase, at least a 3,000% increase, at least a 4,000% increase, at least a 5,000% increase, at least a 6,000% increase, at least a 7,000% increase, at least a 8,000% increase, at a least a 9,000% increase, or at least a 10,000% increase, or about a 1% increase to about 10,000% increase, about a 1% increase to about a 9,000% increase, about a 1% increase to about a 8,000% increase, about a 1% increase to about a 7,000% increase, about a 1% increase to about a 6,000% increase, about a 1% increase to about a 5,000% increase, about a 1% increase to about a 4,000% increase, about a 1% increase to about a 3,000% increase, about a 1% increase to about a 2,000% increase, about a 1% increase to about a 1,000% increase, about a 1% increase to about a 500% increase, about a 1% increase to about a 450% increase, about a 1% increase to about a 400% increase, about a 1% increase to about a 350% increase, about a 1% increase to about a 300% increase, about a 1% increase to about a 250% increase, about a 1% increase to about a 200% increase, about a 1% increase to about a 180% increase, about a 1% increase to about a 160% increase, about a 1% increase to about a 140% increase, about a 1% increase to about a 120% increase, about a 1% increase to about a 100% increase, about a 1% increase to about a 95% increase, about a 1% increase to about a 90% increase, about a 1% increase to about a 85% increase, about a 1% increase to about a 80% increase, about a 1% increase to about a 75% increase, about a 1% increase to about a 70% increase, about a 1% increase to about a 65% increase, about a 1% increase to about a 60% increase, about a 1% increase to about a 55% increase, about a 1% increase to about a 50% increase, about a 1% increase to about a 45% increase, about a 1% increase to about a 40% increase, about a 1% increase to about a 35% increase, about a 1% increase to about a 25% increase, about a 1% increase to about a 20% increase, about a 1% increase to about a 15% increase, about a 1% increase to about a 10% increase, about a 1% increase to about a 5% increase, about a 2% increase to about 10,000% increase, about a 2% increase to about a 9,000% increase, about a 2% increase to about a 8,000% increase, about a 2% increase to about a 7,000% increase, about a 2% increase to about a 6,000% increase, about a 2% increase to about a 5,000% increase, about a 2% increase to about a 4,000% increase, about a 2% increase to about a 3,000% increase, about a 2% increase to about a 2,000% increase, about a 2% increase to about a 1,000% increase, about a 2% increase to about a 500% increase, about a 2% increase to about a 450% increase, about a 2% increase to about a 400% increase, about a 2% increase to about a 350% increase, about a 2% increase to about a 300% increase, about a 2% increase to about a 250% increase, about a 2% increase to about a 200% increase, about a 2% increase to about a 180% increase, about a 2% increase to about a 160% increase, about a 2% increase to about a 140% increase, about a 2% increase to about a 120% increase, about a 2% increase to about a 100% increase, about a 2% increase to about a 95% increase, about a 2% increase to about a 90% increase, about a 2% increase to about a 85% increase, about a 2% increase to about a 80% increase, about a 2% increase to about a 75% increase, about a 2% increase to about a 70% increase, about a 2% increase to about a 65% increase, about a 2% increase to about a 60% increase, about a 2% increase to about a 55% increase, about a 2% increase to about a 50% increase, about a 2% increase to about a 45% increase, about a 2% increase to about a 40% increase, about a 2% increase to about a 35% increase, about a 2% increase to about a 25% increase, about a 2% increase to about a 20% increase, about a 2% increase to about a 15% increase, about a 2% increase to about a 10% increase, about a 2% increase to about a 5% increase, about a 5% increase to about 10,000% increase, about a 5% increase to about a 9,000% increase, about a 5% increase to about a 8,000% increase, about a 5% increase to about a 7,000% increase, about a 5% increase to about a 6,000% increase, about a 5% increase to about a 5,000% increase, about a 5% increase to about a 4,000% increase, about a 5% increase to about a 3,000% increase, about a 5% increase to about a 2,000% increase, about a 5% increase to about a 1,000% increase, about a 5% increase to about a 500% increase, about a 5% increase to about a 450% increase, about a 5% increase to about a 400% increase, about a 5% increase to about a 350% increase, about a 5% increase to about a 300% increase, about a 5% increase to about a 250% increase, about a 5% increase to about a 200% increase, about a 5% increase to about a 180% increase, about a 5% increase to about a 160% increase, about a 5% increase to about a 140% increase, about a 5% increase to about a 120% increase, about a 5% increase to about a 100% increase, about a 5% increase to about a 95% increase, about a 5% increase to about a 90% increase, about a 5% increase to about a 85% increase, about a 5% increase to about a 80% increase, about a 5% increase to about a 75% increase, about a 5% increase to about a 70% increase, about a 5% increase to about a 65% increase, about a 5% increase to about a 60% increase, about a 5% increase to about a 55% increase, about a 5% increase to about a 50% increase, about a 5% increase to about a 45% increase, about a 5% increase to about a 40% increase, about a 5% increase to about a 35% increase, about a 5% increase to about a 25% increase, about a 5% increase to about a 20% increase, about a 5% increase to about a 15% increase, about a 5% increase to about a 10% increase, about a 10% increase to about 10,000% increase, about a 10% increase to about a 9,000% increase, about a 10% increase to about a 8,000% increase, about a 10% increase to about a 7,000% increase, about a 10% increase to about a 6,000% increase, about a 10% increase to about a 5,000% increase, about a 10% increase to about a 4,000% increase, about a 10% increase to about a 3,000% increase, about a 10% increase to about a 2,000% increase, about a 10% increase to about a 1,000% increase, about a 10% increase to about a 500% increase, about a 10% increase to about a 450% increase, about a 10% increase to about a 400% increase, about a 10% increase to about a 350% increase, about a 10% increase to about a 300% increase, about a 10% increase to about a 250% increase, about a 10% increase to about a 200% increase, about a 10% increase to about a 180% increase, about a 10% increase to about a 160% increase, about a 10% increase to about a 140% increase, about a 10% increase to about a 120% increase, about a 10% increase to about a 100% increase, about a 10% increase to about a 95% increase, about a 10% increase to about a 90% increase, about a 10% increase to about a 85% increase, about a 10% increase to about a 80% increase, about a 10% increase to about a 75% increase, about a 10% increase to about a 70% increase, about a 10% increase to about a 65% increase, about a 10% increase to about a 60% increase, about a 10% increase to about a 55% increase, about a 10% increase to about a 50% increase, about a 10% increase to about a 45% increase, about a 10% increase to about a 40% increase, about a 10% increase to about a 35% increase, about a 10% increase to about a 30% increase, about a 10% increase to about a 25% increase, about a 10% increase to about a 20% increase, about a 10% increase to about a 15% increase, about a 15% increase to about 10,000% increase, about a 15% increase to about a 9,000% increase, about a 15% increase to about a 8,000% increase, about a 15% increase to about a 7,000% increase, about a 15% increase to about a 6,000% increase, about a 15% increase to about a 5,000% increase, about a 15% increase to about a 4,000% increase, about a 15% increase to about a 3,000% increase, about a 15% increase to about a 2,000% increase, about a 15% increase to about a 1,000% increase, about a 15% increase to about a 500% increase, about a 15% increase to about a 450% increase, about a 15% increase to about a 400% increase, about a 15% increase to about a 350% increase, about a 15% increase to about a 300% increase, about a 15% increase to about a 250% increase, about a 15% increase to about a 200% increase, about a 15% increase to about a 180% increase, about a 15% increase to about a 160% increase, about a 15% increase to about a 140% increase, about a 15% increase to about a 120% increase, about a 15% increase to about a 100% increase, about a 15% increase to about a 95% increase, about a 15% increase to about a 90% increase, about a 15% increase to about a 85% increase, about a 15% increase to about a 80% increase, about a 15% increase to about a 75% increase, about a 15% increase to about a 70% increase, about a 15% increase to about a 65% increase, about a 15% increase to about a 60% increase, about a 15% increase to about a 55% increase, about a 15% increase to about a 50% increase, about a 15% increase to about a 45% increase, about a 15% increase to about a 40% increase, about a 15% increase to about a 35% increase, about a 15% increase to about a 30% increase, about a 15% increase to about a 25% increase, about a 15% increase to about a 20% increase, about a 20% increase to about 10,000% increase, about a 20% increase to about a 9,000% increase, about a 20% increase to about a 8,000% increase, about a 20% increase to about a 7,000% increase, about a 20% increase to about a 6,000% increase, about a 20% increase to about a 5,000% increase, about a 20% increase to about a 4,000% increase, about a 20% increase to about a 3,000% increase, about a 20% increase to about a 2,000% increase, about a 20% increase to about a 1,000% increase, about a 20% increase to about a 500% increase, about a 20% increase to about a 450% increase, about a 20% increase to about a 400% increase, about a 20% increase to about a 350% increase, about a 20% increase to about a 300% increase, about a 20% increase to about a 250% increase, about a 20% increase to about a 200% increase, about a 20% increase to about a 180% increase, about a 20% increase to about a 160% increase, about a 20% increase to about a 140% increase, about a 20% increase to about a 120% increase, about a 20% increase to about a 100% increase, about a 20% increase to about a 95% increase, about a 20% increase to about a 90% increase, about a 20% increase to about a 85% increase, about a 20% increase to about a 80% increase, about a 20% increase to about a 75% increase, about a 20% increase to about a 70% increase, about a 20% increase to about a 65% increase, about a 20% increase to about a 60% increase, about a 20% increase to about a 55% increase, about a 20% increase to about a 50% increase, about a 20% increase to about a 45% increase, about a 20% increase to about a 40% increase, about a 20% increase to about a 35% increase, about a 20% increase to about a 30% increase, about a 20% increase to about a 25% increase, about a 25% increase to about 10,000% increase, about a 25% increase to about a 9,000% increase, about a 25% increase to about a 8,000% increase, about a 25% increase to about a 7,000% increase, about a 25% increase to about a 6,000% increase, about a 25% increase to about a 5,000% increase, about a 25% increase to about a 4,000% increase, about a 25% increase to about a 3,000% increase, about a 25% increase to about a 2,000% increase, about a 25% increase to about a 1,000% increase, about a 25% increase to about a 500% increase, about a 25% increase to about a 450% increase, about a 25% increase to about a 400% increase, about a 25% increase to about a 350% increase, about a 25% increase to about a 300% increase, about a 25% increase to about a 250% increase, about a 25% increase to about a 200% increase, about a 25% increase to about a 180% increase, about a 25% increase to about a 160% increase, about a 25% increase to about a 140% increase, about a 25% increase to about a 120% increase, about a 25% increase to about a 100% increase, about a 25% increase to about a 95% increase, about a 25% increase to about a 90% increase, about a 25% increase to about a 85% increase, about a 25% increase to about a 80% increase, about a 25% increase to about a 75% increase, about a 25% increase to about a 70% increase, about a 25% increase to about a 65% increase, about a 25% increase to about a 60% increase, about a 25% increase to about a 55% increase, about a 25% increase to about a 50% increase, about a 25% increase to about a 45% increase, about a 25% increase to about a 40% increase, about a 25% increase to about a 35% increase, about a 25% increase to about a 30% increase, about a 30% increase to about 10,000% increase, about a 30% increase to about a 9,000% increase, about a 30% increase to about a 8,000% increase, about a 30% increase to about a 7,000% increase, about a 30% increase to about a 6,000% increase, about a 30% increase to about a 5,000% increase, about a 30% increase to about a 4,000% increase, about a 30% increase to about a 3,000% increase, about a 30% increase to about a 2,000% increase, about a 30% increase to about a 1,000% increase, about a 30% increase to about a 500% increase, about a 30% increase to about a 450% increase, about a 30% increase to about a 400% increase, about a 30% increase to about a 350% increase, about a 30% increase to about a 300% increase, about a 30% increase to about a 250% increase, about a 30% increase to about a 200% increase, about a 30% increase to about a 180% increase, about a 30% increase to about a 160% increase, about a 30% increase to about a 140% increase, about a 30% increase to about a 120% increase, about a 30% increase to about a 100% increase, about a 30% increase to about a 95% increase, about a 30% increase to about a 90% increase, about a 30% increase to about a 85% increase, about a 30% increase to about a 80% increase, about a 30% increase to about a 75% increase, about a 30% increase to about a 70% increase, about a 30% increase to about a 65% increase, about a 30% increase to about a 60% increase, about a 30% increase to about a 55% increase, about a 30% increase to about a 50% increase, about a 30% increase to about a 45% increase, about a 30% increase to about a 40% increase, about a 30% increase to about a 35% increase, about a 35% increase to about 10,000% increase, about a 35% increase to about a 9,000% increase, about a 35% increase to about a 8,000% increase, about a 35% increase to about a 7,000% increase, about a 35% increase to about a 6,000% increase, about a 35% increase to about a 5,000% increase, about a 35% increase to about a 4,000% increase, about a 35% increase to about a 3,000% increase, about a 35% increase to about a 2,000% increase, about a 35% increase to about a 1,000% increase, about a 35% increase to about a 500% increase, about a 35% increase to about a 450% increase, about a 35% increase to about a 400% increase, about a 35% increase to about a 350% increase, about a 35% increase to about a 300% increase, about a 35% increase to about a 250% increase, about a 35% increase to about a 200% increase, about a 35% increase to about a 180% increase, about a 35% increase to about a 160% increase, about a 35% increase to about a 140% increase, about a 35% increase to about a 120% increase, about a 35% increase to about a 100% increase, about a 35% increase to about a 95% increase, about a 35% increase to about a 90% increase, about a 35% increase to about a 85% increase, about a 35% increase to about a 80% increase, about a 35% increase to about a 75% increase, about a 35% increase to about a 70% increase, about a 35% increase to about a 65% increase, about a 35% increase to about a 60% increase, about a 35% increase to about a 55% increase, about a 35% increase to about a 50% increase, about a 35% increase to about a 45% increase, about a 35% increase to about a 40% increase, about a 40% increase to about 10,000% increase, about a 40% increase to about a 9,000% increase, about a 40% increase to about a 8,000% increase, about a 40% increase to about a 7,000% increase, about a 40% increase to about a 6,000% increase, about a 40% increase to about a 5,000% increase, about a 40% increase to about a 4,000% increase, about a 40% increase to about a 3,000% increase, about a 40% increase to about a 2,000% increase, about a 40% increase to about a 1,000% increase, about a 40% increase to about a 500% increase, about a 40% increase to about a 450% increase, about a 40% increase to about a 400% increase, about a 40% increase to about a 350% increase, about a 40% increase to about a 300% increase, about a 40% increase to about a 250% increase, about a 40% increase to about a 200% increase, about a 40% increase to about a 180% increase, about a 40% increase to about a 160% increase, about a 40% increase to about a 140% increase, about a 40% increase to about a 120% increase, about a 40% increase to about a 100% increase, about a 40% increase to about a 95% increase, about a 40% increase to about a 90% increase, about a 40% increase to about a 85% increase, about a 40% increase to about a 80% increase, about a 40% increase to about a 75% increase, about a 40% increase to about a 70% increase, about a 40% increase to about a 65% increase, about a 40% increase to about a 60% increase, about a 40% increase to about a 55% increase, about a 40% increase to about a 50% increase, about a 40% increase to about a 45% increase, about a 45% increase to about 10,000% increase, about a 45% increase to about a 9,000% increase, about a 45% increase to about a 8,000% increase, about a 45% increase to about a 7,000% increase, about a 45% increase to about a 6,000% increase, about a 45% increase to about a 5,000% increase, about a 45% increase to about a 4,000% increase, about a 45% increase to about a 3,000% increase, about a 45% increase to about a 2,000% increase, about a 45% increase to about a 1,000% increase, about a 45% increase to about a 500% increase, about a 45% increase to about a 450% increase, about a 45% increase to about a 400% increase, about a 45% increase to about a 350% increase, about a 45% increase to about a 300% increase, about a 45% increase to about a 250% increase, about a 45% increase to about a 200% increase, about a 45% increase to about a 180% increase, about a 45% increase to about a 160% increase, about a 45% increase to about a 140% increase, about a 45% increase to about a 120% increase, about a 45% increase to about a 100% increase, about a 45% increase to about a 95% increase, about a 45% increase to about a 90% increase, about a 45% increase to about a 85% increase, about a 45% increase to about a 80% increase, about a 45% increase to about a 75% increase, about a 45% increase to about a 70% increase, about a 45% increase to about a 65% increase, about a 45% increase to about a 60% increase, about a 45% increase to about a 55% increase, about a 45% increase to about a 50% increase, about a 50% increase to about 10,000% increase, about a 50% increase to about a 9,000% increase, about a 50% increase to about a 8,000% increase, about a 50% increase to about a 7,000% increase, about a 50% increase to about a 6,000% increase, about a 50% increase to about a 5,000% increase, about a 50% increase to about a 4,000% increase, about a 50% increase to about a 3,000% increase, about a 50% increase to about a 2,000% increase, about a 50% increase to about a 1,000% increase, about a 50% increase to about a 500% increase, about a 50% increase to about a 450% increase, about a 50% increase to about a 400% increase, about a 50% increase to about a 350% increase, about a 50% increase to about a 300% increase, about a 50% increase to about a 250% increase, about a 50% increase to about a 200% increase, about a 50% increase to about a 180% increase, about a 50% increase to about a 160% increase, about a 50% increase to about a 140% increase, about a 50% increase to about a 120% increase, about a 50% increase to about a 100% increase, about a 50% increase to about a 95% increase, about a 50% increase to about a 90% increase, about a 50% increase to about a 85% increase, about a 50% increase to about a 80% increase, about a 50% increase to about a 75% increase, about a 50% increase to about a 70% increase, about a 50% increase to about a 65% increase, about a 50% increase to about a 60% increase, about a 50% increase to about a 55% increase, about a 55% increase to about 10,000% increase, about a 55% increase to about a 9,000% increase, about a 55% increase to about a 8,000% increase, about a 55% increase to about a 7,000% increase, about a 55% increase to about a 6,000% increase, about a 55% increase to about a 5,000% increase, about a 55% increase to about a 4,000% increase, about a 55% increase to about a 3,000% increase, about a 55% increase to about a 2,000% increase, about a 55% increase to about a 1,000% increase, about a 55% increase to about a 500% increase, about a 55% increase to about a 450% increase, about a 55% increase to about a 400% increase, about a 55% increase to about a 350% increase, about a 55% increase to about a 300% increase, about a 55% increase to about a 250% increase, about a 55% increase to about a 200% increase, about a 55% increase to about a 180% increase, about a 55% increase to about a 160% increase, about a 55% increase to about a 140% increase, about a 55% increase to about a 120% increase, about a 55% increase to about a 100% increase, about a 55% increase to about a 95% increase, about a 55% increase to about a 90% increase, about a 55% increase to about a 85% increase, about a 55% increase to about a 80% increase, about a 55% increase to about a 75% increase, about a 55% increase to about a 70% increase, about a 55% increase to about a 65% increase, about a 55% increase to about a 60% increase, about a 60% increase to about 10,000% increase, about a 60% increase to about a 9,000% increase, about a 60% increase to about a 8,000% increase, about a 60% increase to about a 7,000% increase, about a 60% increase to about a 6,000% increase, about a 60% increase to about a 5,000% increase, about a 60% increase to about a 4,000% increase, about a 60% increase to about a 3,000% increase, about a 60% increase to about a 2,000% increase, about a 60% increase to about a 1,000% increase, about a 60% increase to about a 500% increase, about a 60% increase to about a 450% increase, about a 60% increase to about a 400% increase, about a 60% increase to about a 350% increase, about a 60% increase to about a 300% increase, about a 60% increase to about a 250% increase, about a 60% increase to about a 200% increase, about a 60% increase to about a 180% increase, about a 60% increase to about a 160% increase, about a 60% increase to about a 140% increase, about a 60% increase to about a 120% increase, about a 60% increase to about a 100% increase, about a 60% increase to about a 95% increase, about a 60% increase to about a 90% increase, about a 60% increase to about a 85% increase, about a 60% increase to about a 80% increase, about a 60% increase to about a 75% increase, about a 60% increase to about a 70% increase, about a 60% increase to about a 65% increase, about a 65% increase to about 10,000% increase, about a 65% increase to about a 9,000% increase, about a 65% increase to about a 8,000% increase, about a 65% increase to about a 7,000% increase, about a 65% increase to about a 6,000% increase, about a 65% increase to about a 5,000% increase, about a 65% increase to about a 4,000% increase, about a 65% increase to about a 3,000% increase, about a 65% increase to about a 2,000% increase, about a 65% increase to about a 1,000% increase, about a 65% increase to about a 500% increase, about a 65% increase to about a 450% increase, about a 65% increase to about a 400% increase, about a 65% increase to about a 350% increase, about a 65% increase to about a 300% increase, about a 65% increase to about a 250% increase, about a 65% increase to about a 200% increase, about a 65% increase to about a 180% increase, about a 65% increase to about a 160% increase, about a 65% increase to about a 140% increase, about a 65% increase to about a 120% increase, about a 65% increase to about a 100% increase, about a 65% increase to about a 95% increase, about a 65% increase to about a 90% increase, about a 65% increase to about a 85% increase, about a 65% increase to about a 80% increase, about a 65% increase to about a 75% increase, about a 65% increase to about a 70% increase, about a 70% increase to about 10,000% increase, about a 70% increase to about a 9,000% increase, about a 70% increase to about a 8,000% increase, about a 70% increase to about a 7,000% increase, about a 70% increase to about a 6,000% increase, about a 70% increase to about a 5,000% increase, about a 70% increase to about a 4,000% increase, about a 70% increase to about a 3,000% increase, about a 70% increase to about a 2,000% increase, about a 70% increase to about a 1,000% increase, about a 70% increase to about a 500% increase, about a 70% increase to about a 450% increase, about a 70% increase to about a 400% increase, about a 70% increase to about a 350% increase, about a 70% increase to about a 300% increase, about a 70% increase to about a 250% increase, about a 70% increase to about a 200% increase, about a 70% increase to about a 180% increase, about a 70% increase to about a 160% increase, about a 70% increase to about a 140% increase, about a 70% increase to about a 120% increase, about a 70% increase to about a 100% increase, about a 70% increase to about a 95% increase, about a 70% increase to about a 90% increase, about a 70% increase to about a 85% increase, about a 70% increase to about a 80% increase, about a 70% increase to about a 75% increase, about a 75% increase to about 10,000% increase, about a 75% increase to about a 9,000% increase, about a 75% increase to about a 8,000% increase, about a 75% increase to about a 7,000% increase, about a 75% increase to about a 6,000% increase, about a 75% increase to about a 5,000% increase, about a 75% increase to about a 4,000% increase, about a 75% increase to about a 3,000% increase, about a 75% increase to about a 2,000% increase, about a 75% increase to about a 1,000% increase, about a 75% increase to about a 500% increase, about a 75% increase to about a 450% increase, about a 75% increase to about a 400% increase, about a 75% increase to about a 350% increase, about a 75% increase to about a 300% increase, about a 75% increase to about a 250% increase, about a 75% increase to about a 200% increase, about a 75% increase to about a 180% increase, about a 75% increase to about a 160% increase, about a 75% increase to about a 140% increase, about a 75% increase to about a 120% increase, about a 75% increase to about a 100% increase, about a 75% increase to about a 95% increase, about a 75% increase to about a 90% increase, about a 75% increase to about a 85% increase, about a 75% increase to about a 80%, about a 80% increase to about 10,000% increase, about a 80% increase to about a 9,000% increase, about a 80% increase to about a 8,000% increase, about a 80% increase to about a 7,000% increase, about a 80% increase to about a 6,000% increase, about a 80% increase to about a 5,000% increase, about a 80% increase to about a 4,000% increase, about a 80% increase to about a 3,000% increase, about a 80% increase to about a 2,000% increase, about a 80% increase to about a 1,000% increase, increase, about a 80% increase to about a 500% increase, about a 80% increase to about a 450% increase, about a 80% increase to about a 400% increase, about a 80% increase to about a 350% increase, about a 80% increase to about a 300% increase, about a 80% increase to about a 250% increase, about a 80% increase to about a 200% increase, about a 80% increase to about a 180% increase, about a 80% increase to about a 160% increase, about a 80% increase to about a 140% increase, about a 80% increase to about a 120% increase, about a 80% increase to about a 100% increase, about a 80% increase to about a 95% increase, about a 80% increase to about a 90% increase, about a 80% increase to about a 85% increase, about a 85% increase to about 10,000% increase, about a 85% increase to about a 9,000% increase, about a 85% increase to about a 8,000% increase, about a 85% increase to about a 7,000% increase, about a 85% increase to about a 6,000% increase, about a 85% increase to about a 5,000% increase, about a 85% increase to about a 4,000% increase, about a 85% increase to about a 3,000% increase, about a 85% increase to about a 2,000% increase, about a 85% increase to about a 1,000% increase, about a 85% increase to about a 500% increase, about a 85% increase to about a 450% increase, about a 85% increase to about a 400% increase, about a 85% increase to about a 350% increase, about a 85% increase to about a 300% increase, about a 85% increase to about a 250% increase, about a 85% increase to about a 200% increase, about a 85% increase to about a 180% increase, about a 85% increase to about a 160% increase, about a 85% increase to about a 140% increase, about a 85% increase to about a 120% increase, about a 85% increase to about a 100% increase, about a 85% increase to about a 95% increase, about a 85% increase to about a 90% increase, about a 90% increase to about 10,000% increase, about a 90% increase to about a 9,000% increase, about a 90% increase to about a 8,000% increase, about a 90% increase to about a 7,000% increase, about a 90% increase to about a 6,000% increase, about a 90% increase to about a 5,000% increase, about a 90% increase to about a 4,000% increase, about a 90% increase to about a 3,000% increase, about a 90% increase to about a 2,000% increase, about a 90% increase to about a 1,000% increase, about a 90% increase to about a 500% increase, about a 90% increase to about a 450% increase, about a 90% increase to about a 400% increase, about a 90% increase to about a 350% increase, about a 90% increase to about a 300% increase, about a 90% increase to about a 250% increase, about a 90% increase to about a 200% increase, about a 90% increase to about a 180% increase, about a 90% increase to about a 160% increase, about a 90% increase to about a 140% increase, about a 90% increase to about a 120% increase, about a 90% increase to about a 100% increase, about a 90% increase to about a 95% increase, about a 95% increase to about 10,000% increase, about a 95% increase to about a 9,000% increase, about a 95% increase to about a 8,000% increase, about a 95% increase to about a 7,000% increase, about a 95% increase to about a 6,000% increase, about a 95% increase to about a 5,000% increase, about a 95% increase to about a 4,000% increase, about a 95% increase to about a 3,000% increase, about a 95% increase to about a 2,000% increase, about a 95% increase to about a 1,000% increase, about a 95% increase to about a 500% increase, about a 95% increase to about a 450% increase, about a 95% increase to about a 400% increase, about a 95% increase to about a 350% increase, about a 95% increase to about a 300% increase, about a 95% increase to about a 250% increase, about a 95% increase to about a 200% increase, about a 95% increase to about a 180% increase, about a 95% increase to about a 160% increase, about a 95% increase to about a 140% increase, about a 95% increase to about a 120% increase, about a 95% increase to about a 100% increase, about a 100% increase to about 10,000% increase, about a 100% increase to about a 9,000% increase, about a 100% increase to about a 8,000% increase, about a 100% increase to about a 7,000% increase, about a 100% increase to about a 6,000% increase, about a 100% increase to about a 5,000% increase, about a 100% increase to about a 4,000% increase, about a 100% increase to about a 3,000% increase, about a 100% increase to about a 2,000% increase, about a 100% increase to about a 1,000% increase, about a 100% increase to about a 500% increase, about a 100% increase to about a 450% increase, about a 100% increase to about a 400% increase, about a 100% increase to about a 350% increase, about a 100% increase to about a 300% increase, about a 100% increase to about a 250% increase, about a 100% increase to about a 200% increase, about a 100% increase to about a 180% increase, about a 100% increase to about a 160% increase, about a 100% increase to about a 140% increase, about a 100% increase to about a 120% increase, about a 120% increase to about 10,000% increase, about a 120% increase to about a 9,000% increase, about a 120% increase to about a 8,000% increase, about a 120% increase to about a 7,000% increase, about a 120% increase to about a 6,000% increase, about a 120% increase to about a 5,000% increase, about a 120% increase to about a 4,000% increase, about a 120% increase to about a 3,000% increase, about a 120% increase to about a 2,000% increase, about a 120% increase to about a 1,000% increase, about a 120% increase to about a 500% increase, about a 120% increase to about a 450% increase, about a 120% increase to about a 400% increase, about a 120% increase to about a 350% increase, about a 120% increase to about a 300% increase, about a 120% increase to about a 250% increase, about a 120% increase to about a 200% increase, about a 120% increase to about a 180% increase, about a 120% increase to about a 160% increase, about a 120% increase to about a 140% increase, about a 140% increase to about 10,000% increase, about a 140% increase to about a 9,000% increase, about a 140% increase to about a 8,000% increase, about a 140% increase to about a 7,000% increase, about a 140% increase to about a 6,000% increase, about a 140% increase to about a 5,000% increase, about a 140% increase to about a 4,000% increase, about a 140% increase to about a 3,000% increase, about a 140% increase to about a 2,000% increase, about a 140% increase to about a 1,000% increase, about a 140% increase to about a 500% increase, about a 140% increase to about a 450% increase, about a 140% increase to about a 400% increase, about a 140% increase to about a 350% increase, about a 140% increase to about a 300% increase, about a 140% increase to about a 250% increase, about a 140% increase to about a 200% increase, about a 140% increase to about a 180% increase, about a 140% increase to about a 160% increase, about a 160% increase to about 10,000% increase, about a 160% increase to about a 9,000% increase, about a 160% increase to about a 8,000% increase, about a 160% increase to about a 7,000% increase, about a 160% increase to about a 6,000% increase, about a 160% increase to about a 5,000% increase, about a 160% increase to about a 4,000% increase, about a 160% increase to about a 3,000% increase, about a 160% increase to about a 2,000% increase, about a 160% increase to about a 1,000% increase, about a 160% increase to about a 500% increase, about a 160% increase to about a 450% increase, about a 160% increase to about a 400% increase, about a 160% increase to about a 350% increase, about a 160% increase to about a 300% increase, about a 160% increase to about a 250% increase, about a 160% increase to about a 200% increase, about a 160% increase to about a 180% increase, about a 180% increase to about 10,000% increase, about a 180% increase to about a 9,000% increase, about a 180% increase to about a 8,000% increase, about a 180% increase to about a 7,000% increase, about a 180% increase to about a 6,000% increase, about a 180% increase to about a 5,000% increase, about a 180% increase to about a 4,000% increase, about a 180% increase to about a 3,000% increase, about a 180% increase to about a 2,000% increase, about a 180% increase to about a 1,000% increase, about a 180% increase to about a 500% increase, about a 180% increase to about a 450% increase, about a 180% increase to about a 400% increase, about a 180% increase to about a 350% increase, about a 180% increase to about a 300% increase, about a 180% increase to about a 250% increase, about a 180% increase to about a 200% increase, about a 200% increase to about 10,000% increase, about a 200% increase to about a 9,000% increase, about a 200% increase to about a 8,000% increase, about a 200% increase to about a 7,000% increase, about a 200% increase to about a 6,000% increase, about a 200% increase to about a 5,000% increase, about a 200% increase to about a 4,000% increase, about a 200% increase to about a 3,000% increase, about a 200% increase to about a 2,000% increase, about a 200% increase to about a 1,000% increase, about a 200% increase to about a 500% increase, about a 200% increase to about a 450% increase, about a 200% increase to about a 400% increase, about a 200% increase to about a 350% increase, about a 200% increase to about a 300% increase, about a 200% increase to about a 250% increase, about a 250% increase to about 10,000% increase, about a 250% increase to about a 9,000% increase, about a 250% increase to about a 8,000% increase, about a 250% increase to about a 7,000% increase, about a 250% increase to about a 6,000% increase, about a 250% increase to about a 5,000% increase, about a 250% increase to about a 4,000% increase, about a 250% increase to about a 3,000% increase, about a 250% increase to about a 2,000% increase, about a 250% increase to about a 1,000% increase, about a 250% increase to about a 500% increase, about a 250% increase to about a 450% increase, about a 250% increase to about a 400% increase, about a 250% increase to about a 350% increase, about a 250% increase to about a 300% increase, about a 300% increase to about 10,000% increase, about a 300% increase to about a 9,000% increase, about a 300% increase to about a 8,000% increase, about a 300% increase to about a 7,000% increase, about a 300% increase to about a 6,000% increase, about a 300% increase to about a 5,000% increase, about a 300% increase to about a 4,000% increase, about a 300% increase to about a 3,000% increase, about a 300% increase to about a 2,000% increase, about a 300% increase to about a 1,000% increase, about a 300% increase to about a 500% increase, about a 300% increase to about a 450% increase, about a 300% increase to about a 400% increase, about a 300% increase to about a 350% increase, about a 350% increase to about 10,000% increase, about a 350% increase to about a 9,000% increase, about a 350% increase to about a 8,000% increase, about a 350% increase to about a 7,000% increase, about a 350% increase to about a 6,000% increase, about a 350% increase to about a 5,000% increase, about a 350% increase to about a 4,000% increase, about a 350% increase to about a 3,000% increase, about a 350% increase to about a 2,000% increase, about a 350% increase to about a 1,000% increase, about a 350% increase to about a 500% increase, about a 350% increase to about a 450% increase, about a 350% increase to about a 400% increase, about a 400% increase to about 10,000% increase, about a 400% increase to about a 9,000% increase, about a 400% increase to about a 8,000% increase, about a 400% increase to about a 7,000% increase, about a 400% increase to about a 6,000% increase, about a 400% increase to about a 5,000% increase, about a 400% increase to about a 4,000% increase, about a 400% increase to about a 3,000% increase, about a 400% increase to about a 2,000% increase, about a 400% increase to about a 1,000% increase, about a 400% increase to about a 500% increase, about a 400% increase to about a 450% increase, about a 450% increase to about 10,000% increase, about a 450% increase to about a 9,000% increase, about a 450% increase to about a 8,000% increase, about a 450% increase to about a 7,000% increase, about a 450% increase to about a 6,000% increase, about a 450% increase to about a 5,000% increase, about a 450% increase to about a 4,000% increase, about a 450% increase to about a 3,000% increase, about a 450% increase to about a 2,000% increase, about a 450% increase to about a 1,000% increase, about a 450% increase to about a 500% increase, about a 500% increase to about 10,000% increase, about a 500% increase to about a 9,000% increase, about a 500% increase to about a 8,000% increase, about a 500% increase to about a 7,000% increase, about a 500% increase to about a 6,000% increase, about a 500% increase to about a 5,000% increase, about a 500% increase to about a 4,000% increase, about a 500% increase to about a 3,000% increase, about a 500% increase to about a 2,000% increase, about a 500% increase to about a 1,000% increase, about a 1,000% increase to about 10,000% increase, about a 1,000% increase to about a 9,000% increase, about a 1,000% increase to about a 8,000% increase, about a 1,000% increase to about a 7,000% increase, about a 1,000% increase to about a 6,000% increase, about a 1,000% increase to about a 5,000% increase, about a 1,000% increase to about a 4,000% increase, about a 1,000% increase to about a 3,000% increase, about a 1,000% increase to about a 2,000% increase, about a 2,000% increase to about 10,000% increase, about a 2,000% increase to about a 9,000% increase, about a 2,000% increase to about a 8,000% increase, about a 2,000% increase to about a 7,000% increase, about a 2,000% increase to about a 6,000% increase, about a 2,000% increase to about a 5,000% increase, about a 2,000% increase to about a 4,000% increase, about a 2,000% increase to about a 3,000% increase, about a 3,000% increase to about 10,000% increase, about a 3,000% increase to about a 9,000% increase, about a 3,000% increase to about a 8,000% increase, about a 3,000% increase to about a 7,000% increase, about a 3,000% increase to about a 6,000% increase, about a 3,000% increase to about a 5,000% increase, about a 3,000% increase to about a 4,000% increase, about a 4,000% increase to about 10,000% increase, about a 4,000% increase to about a 9,000% increase, about a 4,000% increase to about a 8,000% increase, about a 4,000% increase to about a 7,000% increase, about a 4,000% increase to about a 6,000% increase, about a 4,000% increase to about a 5,000% increase, about a 5,000% increase to about 10,000% increase, about a 5,000% increase to about a 9,000% increase, about a 5,000% increase to about a 8,000% increase, about a 5,000% increase to about a 7,000% increase, about a 5,000% increase to about a 6,000% increase, about a 6,000% increase to about 10,000% increase, about a 6,000% increase to about a 9,000% increase, about a 6,000% increase to about a 8,000% increase, about a 6,000% increase to about a 7,000% increase, about a 7,000% increase to about 10,000% increase, about a 7,000% increase to about a 9,000% increase, about a 7,000% increase to about a 8,000% increase, about a 8,000% increase to about 10,000% increase, about a 8,000% increase to about a 9,000% increase, or about a 9,000% increase to about 10,000%) in toxin liberation in the target mammalian cell (e.g., any of the target mammalian cells described herein) as compared to a composition including the same amount of a control ABPC (e.g., any of the exemplary control ABPCs described herein).

In some examples of any of the ABPCs described herein, a composition including the ABPC (e.g., any of the ABPCs described herein) can provide for an increase (e.g., a detectable increase) (e.g., at least a 0.1-fold increase, at least a 0.2-fold increase, at least a 0.3-fold increase, at least a 0.4-fold increase, at least a 0.5-fold increase, at least a 0.6-fold increase, at least a 0.7-fold increase, at least a 0.8-fold increase, at least a 0.9-fold increase, at least a 1.0-fold increase, at least a 1.2-fold increase, at least a 1.4-fold increase, at least a 1.5-fold increase, at least a 1.6-fold increase, at least a 1.8-fold increase, at least a 2.0-fold increase, at least a 2.2-fold increase, at least a 2.4-fold increase, at least a 2.5-fold increase, at least a 2.6-fold increase, at least a 2.8-fold increase, at least a 3.0-fold increase, at least a 3.5-fold increase, at least a 4.0-fold increase, at least a 4.5-fold increase, at least a 5.0-fold increase, at least a 5.5-fold increase, at least a 6.0-fold increase, at least a 6.5-fold increase, at least a 7.0-fold increase, at least a 7.5-fold increase, at least a 8.0-fold increase, at least a 8.5-fold increase, at least a 9.0-fold increase, at least a 9.5-fold increase, at least a 10-fold increase, at least a 15-fold increase, at least a 20-fold increase, at least a 25-fold increase, at least a 30-fold increase, at least a 35-fold increase, at least a 40-fold increase, at least a 45-fold increase, at least a 50-fold increase, at least a 55-fold increase, at least a 60-fold increase, at least a 65-fold increase, at least a 70-fold increase, at least a 75-fold increase, at least a 80-fold increase, at least a 85-fold increase, at least a 90-fold increase, at least a 95-fold increase, or at least a 100-fold increase, or about a 0.1-fold increase to about a 100-fold increase, about 0.1-fold increase to about a 90-fold increase, about 0.1-fold increase to about a 80-fold increase, about a 0.1-fold increase to about a 70-fold increase, about a 0.1-fold increase to about a 60-fold increase, about a 0.1-fold increase to about a 50-fold increase, about a 0.1-fold increase to about a 40-fold increase, about a 0.1-fold increase to about a 30-fold increase, about 0.1-fold increase to about 20-fold increase, about a 0.1-fold increase to about a 10-fold increase, about a 0.1-fold increase to about a 9.5-fold increase, about a 0.1-fold increase to about a 9.0-fold increase, about a 0.1-fold increase to about a 8.5-fold increase, about a 0.1-fold increase to about a 8.0-fold increase, about a 0.1-fold increase to about a 7.5-fold increase, about a 0.1-fold increase to about a 7.0-fold increase, about a 0.1-fold increase to about a 6.5-fold increase, about a 0.1-fold increase to about a 6.0-fold increase, about a 0.1-fold increase to about a 5.5-fold increase, about a 0.1-fold increase to about a 5.0-fold increase, about a 0.1-fold increase to about a 4.5-fold increase, about a 0.1-fold increase to about a 4.0-fold increase, about a 0.1-fold increase to about a 3.5-fold increase, about 0.1-fold increase to about a 3.0-fold increase, about a 0.1-fold increase to about a 2.8-fold increase, about a 0.1-fold increase to about a 2.6-fold increase, about a 0.1-fold increase to about a 2.5-fold increase, about a 0.1-fold increase to about a 2.4-fold increase, about a 0.1-fold increase to about a 2.2-fold increase, about a 0.1-fold increase to about a 2.0-fold increase, about a 0.1-fold increase to about a 1.8-fold increase, about a 0.1-fold increase to about a 1.6-fold increase, about a 0.1-fold increase to about a 1.5-fold increase, about a 0.1-fold increase to about a 1.4-fold increase, about a 0.1-fold increase to about a 1.2-fold increase, about a 0.1-fold increase to about a 1.0-fold increase, about a 0.1-fold increase to about a 0.9-fold increase, about a 0.1-fold increase to about a 0.8-fold increase, about a 0.1-fold increase to about a 0.7-fold increase, about a 0.1-fold increase to about a 0.6-fold increase, about a 0.1-fold increase to about a 0.5-fold increase, about a 0.1-fold increase to about a 0.4-fold increase, about a 0.1-fold increase to about a 0.3-fold increase, about a 0.2-fold increase to about a 100-fold increase, about 0.2-fold increase to about a 90-fold increase, about 0.2-fold increase to about a 80-fold increase, about a 0.2-fold increase to about a 70-fold increase, about a 0.2-fold increase to about a 60-fold increase, about a 0.2-fold increase to about a 50-fold increase, about a 0.2-fold increase to about a 40-fold increase, about a 0.2-fold increase to about a 30-fold increase, about 0.2-fold increase to about 20-fold increase, about a 0.2-fold increase to about a 10-fold increase, about a 0.2-fold increase to about a 9.5-fold increase, about a 0.2-fold increase to about a 9.0-fold increase, about a 0.2-fold increase to about a 8.5-fold increase, about a 0.2-fold increase to about a 8.0-fold increase, about a 0.2-fold increase to about a 7.5-fold increase, about a 0.2-fold increase to about a 7.0-fold increase, about a 0.2-fold increase to about a 6.5-fold increase, about a 0.2-fold increase to about a 6.0-fold increase, about a 0.2-fold increase to about a 5.5-fold increase, about a 0.2-fold increase to about a 5.0-fold increase, about a 0.2-fold increase to about a 4.5-fold increase, about a 0.2-fold increase to about a 4.0-fold increase, about a 0.2-fold increase to about a 3.5-fold increase, about 0.2-fold increase to about a 3.0-fold increase, about a 0.2-fold increase to about a 2.8-fold increase, about a 0.2-fold increase to about a 2.6-fold increase, about a 0.2-fold increase to about a 2.5-fold increase, about a 0.2-fold increase to about a 2.4-fold increase, about a 0.2-fold increase to about a 2.2-fold increase, about a 0.2-fold increase to about a 2.0-fold increase, about a 0.2-fold increase to about a 1.8-fold increase, about a 0.2-fold increase to about a 1.6-fold increase, about a 0.2-fold increase to about a 1.5-fold increase, about a 0.2-fold increase to about a 1.4-fold increase, about a 0.2-fold increase to about a 1.2-fold increase, about a 0.2-fold increase to about a 1.0-fold increase, about a 0.2-fold increase to about a 0.9-fold increase, about a 0.2-fold increase to about a 0.8-fold increase, about a 0.2-fold increase to about a 0.7-fold increase, about a 0.2-fold increase to about a 0.6-fold increase, about a 0.2-fold increase to about a 0.5-fold increase, about a 0.2-fold increase to about a 0.4-fold increase, about a 0.3-fold increase to about a 100-fold increase, about 0.3-fold increase to about a 90-fold increase, about 0.3-fold increase to about a 80-fold increase, about a 0.3-fold increase to about a 70-fold increase, about a 0.3-fold increase to about a 60-fold increase, about a 0.3-fold increase to about a 50-fold increase, about a 0.3-fold increase to about a 40-fold increase, about a 0.3-fold increase to about a 30-fold increase, about 0.3-fold increase to about 20-fold increase, about a 0.3-fold increase to about a 10-fold increase, about a 0.3-fold increase to about a 9.5-fold increase, about a 0.3-fold increase to about a 9.0-fold increase, about a 0.3-fold increase to about a 8.5-fold increase, about a 0.3-fold increase to about a 8.0-fold increase, about a 0.3-fold increase to about a 7.5-fold increase, about a 0.3-fold increase to about a 7.0-fold increase, about a 0.3-fold increase to about a 6.5-fold increase, about a 0.3-fold increase to about a 6.0-fold increase, about a 0.3-fold increase to about a 5.5-fold increase, about a 0.3-fold increase to about a 5.0-fold increase, about a 0.3-fold increase to about a 4.5-fold increase, about a 0.3-fold increase to about a 4.0-fold increase, about a 0.3-fold increase to about a 3.5-fold increase, about 0.3-fold increase to about a 3.0-fold increase, about a 0.3-fold increase to about a 2.8-fold increase, about a 0.3-fold increase to about a 2.6-fold increase, about a 0.3-fold increase to about a 2.5-fold increase, about a 0.3-fold increase to about a 2.4-fold increase, about a 0.3-fold increase to about a 2.2-fold increase, about a 0.3-fold increase to about a 2.0-fold increase, about a 0.3-fold increase to about a 1.8-fold increase, about a 0.3-fold increase to about a 1.6-fold increase, about a 0.3-fold increase to about a 1.5-fold increase, about a 0.3-fold increase to about a 1.4-fold increase, about a 0.3-fold increase to about a 1.2-fold increase, about a 0.3-fold increase to about a 1.0-fold increase, about a 0.3-fold increase to about a 0.9-fold increase, about a 0.3-fold increase to about a 0.8-fold increase, about a 0.3-fold increase to about a 0.7-fold increase, about a 0.3-fold increase to about a 0.6-fold increase, about a 0.3-fold increase to about a 0.5-fold increase, about a 0.4-fold increase to about a 100-fold increase, about 0.4-fold increase to about a 90-fold increase, about 0.4-fold increase to about a 80-fold increase, about a 0.4-fold increase to about a 70-fold increase, about a 0.4-fold increase to about a 60-fold increase, about a 0.4-fold increase to about a 50-fold increase, about a 0.4-fold increase to about a 40-fold increase, about a 0.4-fold increase to about a 30-fold increase, about 0.4-fold increase to about 20-fold increase, about a 0.4-fold increase to about a 10-fold increase, about a 0.4-fold increase to about a 9.5-fold increase, about a 0.4-fold increase to about a 9.0-fold increase, about a 0.4-fold increase to about a 8.5-fold increase, about a 0.4-fold increase to about a 8.0-fold increase, about a 0.4-fold increase to about a 7.5-fold increase, about a 0.4-fold increase to about a 7.0-fold increase, about a 0.4-fold increase to about a 6.5-fold increase, about a 0.4-fold increase to about a 6.0-fold increase, about a 0.4-fold increase to about a 5.5-fold increase, about a 0.4-fold increase to about a 5.0-fold increase, about a 0.4-fold increase to about a 4.5-fold increase, about a 0.4-fold increase to about a 4.0-fold increase, about a 0.4-fold increase to about a 3.5-fold increase, about 0.4-fold increase to about a 3.0-fold increase, about a 0.4-fold increase to about a 2.8-fold increase, about a 0.4-fold increase to about a 2.6-fold increase, about a 0.4-fold increase to about a 2.5-fold increase, about a 0.4-fold increase to about a 2.4-fold increase, about a 0.4-fold increase to about a 2.2-fold increase, about a 0.4-fold increase to about a 2.0-fold increase, about a 0.4-fold increase to about a 1.8-fold increase, about a 0.4-fold increase to about a 1.6-fold increase, about a 0.4-fold increase to about a 1.5-fold increase, about a 0.4-fold increase to about a 1.4-fold increase, about a 0.4-fold increase to about a 1.2-fold increase, about a 0.4-fold increase to about a 1.0-fold increase, about a 0.4-fold increase to about a 0.9-fold increase, about a 0.4-fold increase to about a 0.8-fold increase, about a 0.4-fold increase to about a 0.7-fold increase, about a 0.4-fold increase to about a 0.6-fold increase, about a 0.5-fold increase to about a 100-fold increase, about 0.5-fold increase to about a 90-fold increase, about 0.5-fold increase to about a 80-fold increase, about a 0.5-fold increase to about a 70-fold increase, about a 0.5-fold increase to about a 60-fold increase, about a 0.5-fold increase to about a 50-fold increase, about a 0.5-fold increase to about a 40-fold increase, about a 0.5-fold increase to about a 30-fold increase, about 0.5-fold increase to about 20-fold increase, about a 0.5-fold increase to about a 10-fold increase, about a 0.5-fold increase to about a 9.5-fold increase, about a 0.5-fold increase to about a 9.0-fold increase, about a 0.5-fold increase to about a 8.5-fold increase, about a 0.5-fold increase to about a 8.0-fold increase, about a 0.5-fold increase to about a 7.5-fold increase, about a 0.5-fold increase to about a 7.0-fold increase, about a 0.5-fold increase to about a 6.5-fold increase, about a 0.5-fold increase to about a 6.0-fold increase, about a 0.5-fold increase to about a 5.5-fold increase, about a 0.5-fold increase to about a 5.0-fold increase, about a 0.5-fold increase to about a 4.5-fold increase, about a 0.5-fold increase to about a 4.0-fold increase, about a 0.5-fold increase to about a 3.5-fold increase, about 0.5-fold increase to about a 3.0-fold increase, about a 0.5-fold increase to about a 2.8-fold increase, about a 0.5-fold increase to about a 2.6-fold increase, about a 0.5-fold increase to about a 2.5-fold increase, about a 0.5-fold increase to about a 2.4-fold increase, about a 0.5-fold increase to about a 2.2-fold increase, about a 0.5-fold increase to about a 2.0-fold increase, about a 0.5-fold increase to about a 1.8-fold increase, about a 0.5-fold increase to about a 1.6-fold increase, about a 0.5-fold increase to about a 1.5-fold increase, about a 0.5-fold increase to about a 1.4-fold increase, about a 0.5-fold increase to about a 1.2-fold increase, about a 0.5-fold increase to about a 1.0-fold increase, about a 0.5-fold increase to about a 0.9-fold increase, about a 0.5-fold increase to about a 0.8-fold increase, about a 0.5-fold increase to about a 0.7-fold increase, about a 0.6-fold increase to about a 100-fold increase, about 0.6-fold increase to about a 90-fold increase, about 0.6-fold increase to about a 80-fold increase, about a 0.6-fold increase to about a 70-fold increase, about a 0.6-fold increase to about a 60-fold increase, about a 0.6-fold increase to about a 50-fold increase, about a 0.6-fold increase to about a 40-fold increase, about a 0.6-fold increase to about a 30-fold increase, about 0.6-fold increase to about 20-fold increase, about a 0.6-fold increase to about a 10-fold increase, about a 0.6-fold increase to about a 9.5-fold increase, about a 0.6-fold increase to about a 9.0-fold increase, about a 0.6-fold increase to about a 8.5-fold increase, about a 0.6-fold increase to about a 8.0-fold increase, about a 0.6-fold increase to about a 7.5-fold increase, about a 0.6-fold increase to about a 7.0-fold increase, about a 0.6-fold increase to about a 6.5-fold increase, about a 0.6-fold increase to about a 6.0-fold increase, about a 0.6-fold increase to about a 5.5-fold increase, about a 0.6-fold increase to about a 5.0-fold increase, about a 0.6-fold increase to about a 4.5-fold increase, about a 0.6-fold increase to about a 4.0-fold increase, about a 0.6-fold increase to about a 3.5-fold increase, about 0.6-fold increase to about a 3.0-fold increase, about a 0.6-fold increase to about a 2.8-fold increase, about a 0.6-fold increase to about a 2.6-fold increase, about a 0.6-fold increase to about a 2.5-fold increase, about a 0.6-fold increase to about a 2.4-fold increase, about a 0.6-fold increase to about a 2.2-fold increase, about a 0.6-fold increase to about a 2.0-fold increase, about a 0.6-fold increase to about a 1.8-fold increase, about a 0.6-fold increase to about a 1.6-fold increase, about a 0.6-fold increase to about a 1.5-fold increase, about a 0.6-fold increase to about a 1.4-fold increase, about a 0.6-fold increase to about a 1.2-fold increase, about a 0.6-fold increase to about a 1.0-fold increase, about a 0.6-fold increase to about a 0.9-fold increase, about a 0.6-fold increase to about a 0.8-fold increase, about a 0.7-fold increase to about a 100-fold increase, about 0.7-fold increase to about a 90-fold increase, about 0.7-fold increase to about a 80-fold increase, about a 0.7-fold increase to about a 70-fold increase, about a 0.7-fold increase to about a 60-fold increase, about a 0.7-fold increase to about a 50-fold increase, about a 0.7-fold increase to about a 40-fold increase, about a 0.7-fold increase to about a 30-fold increase, about 0.7-fold increase to about 20-fold increase, about a 0.7-fold increase to about a 10-fold increase, about a 0.7-fold increase to about a 9.5-fold increase, about a 0.7-fold increase to about a 9.0-fold increase, about a 0.7-fold increase to about a 8.5-fold increase, about a 0.7-fold increase to about a 8.0-fold increase, about a 0.7-fold increase to about a 7.5-fold increase, about a 0.7-fold increase to about a 7.0-fold increase, about a 0.7-fold increase to about a 6.5-fold increase, about a 0.7-fold increase to about a 6.0-fold increase, about a 0.7-fold increase to about a 5.5-fold increase, about a 0.7-fold increase to about a 5.0-fold increase, about a 0.7-fold increase to about a 4.5-fold increase, about a 0.7-fold increase to about a 4.0-fold increase, about a 0.7-fold increase to about a 3.5-fold increase, about 0.7-fold increase to about a 3.0-fold increase, about a 0.7-fold increase to about a 2.8-fold increase, about a 0.7-fold increase to about a 2.6-fold increase, about a 0.7-fold increase to about a 2.5-fold increase, about a 0.7-fold increase to about a 2.4-fold increase, about a 0.7-fold increase to about a 2.2-fold increase, about a 0.7-fold increase to about a 2.0-fold increase, about a 0.7-fold increase to about a 1.8-fold increase, about a 0.7-fold increase to about a 1.6-fold increase, about a 0.7-fold increase to about a 1.5-fold increase, about a 0.7-fold increase to about a 1.4-fold increase, about a 0.7-fold increase to about a 1.2-fold increase, about a 0.7-fold increase to about a 1.0-fold increase, about a 0.7-fold increase to about a 0.9-fold increase, about a 0.8-fold increase to about a 100-fold increase, about 0.8-fold increase to about a 90-fold increase, about 0.8-fold increase to about a 80-fold increase, about a 0.8-fold increase to about a 70-fold increase, about a 0.8-fold increase to about a 60-fold increase, about a 0.8-fold increase to about a 50-fold increase, about a 0.8-fold increase to about a 40-fold increase, about a 0.8-fold increase to about a 30-fold increase, about 0.8-fold increase to about 20-fold increase, about a 0.8-fold increase to about a 10-fold increase, about a 0.8-fold increase to about a 9.5-fold increase, about a 0.8-fold increase to about a 9.0-fold increase, about a 0.8-fold increase to about a 8.5-fold increase, about a 0.8-fold increase to about a 8.0-fold increase, about a 0.8-fold increase to about a 7.5-fold increase, about a 0.8-fold increase to about a 7.0-fold increase, about a 0.8-fold increase to about a 6.5-fold increase, about a 0.8-fold increase to about a 6.0-fold increase, about a 0.8-fold increase to about a 5.5-fold increase, about a 0.8-fold increase to about a 5.0-fold increase, about a 0.8-fold increase to about a 4.5-fold increase, about a 0.8-fold increase to about a 4.0-fold increase, about a 0.8-fold increase to about a 3.5-fold increase, about 0.8-fold increase to about a 3.0-fold increase, about a 0.8-fold increase to about a 2.8-fold increase, about a 0.8-fold increase to about a 2.6-fold increase, about a 0.8-fold increase to about a 2.5-fold increase, about a 0.8-fold increase to about a 2.4-fold increase, about a 0.8-fold increase to about a 2.2-fold increase, about a 0.8-fold increase to about a 2.0-fold increase, about a 0.8-fold increase to about a 1.8-fold increase, about a 0.8-fold increase to about a 1.6-fold increase, about a 0.8-fold increase to about a 1.5-fold increase, about a 0.8-fold increase to about a 1.4-fold increase, about a 0.8-fold increase to about a 1.2-fold increase, about a 0.8-fold increase to about a 1.0-fold increase, about a 1.0-fold increase to about a 100-fold increase, about 1.0-fold increase to about a 90-fold increase, about 1.0-fold increase to about a 80-fold increase, about a 1.0-fold increase to about a 70-fold increase, about a 1.0-fold increase to about a 60-fold increase, about a 1.0-fold increase to about a 50-fold increase, about a 1.0-fold increase to about a 40-fold increase, about a 1.0-fold increase to about a 30-fold increase, about 1.0-fold increase to about 20-fold increase, about a 1.0-fold increase to about a 10-fold increase, about a 1.0-fold increase to about a 9.5-fold increase, about a 1.0-fold increase to about a 9.0-fold increase, about a 1.0-fold increase to about a 8.5-fold increase, about a 1.0-fold increase to about a 8.0-fold increase, about a 1.0-fold increase to about a 7.5-fold increase, about a 1.0-fold increase to about a 7.0-fold increase, about a 1.0-fold increase to about a 6.5-fold increase, about a 1.0-fold increase to about a 6.0-fold increase, about a 1.0-fold increase to about a 5.5-fold increase, about a 1.0-fold increase to about a 5.0-fold increase, about a 1.0-fold increase to about a 4.5-fold increase, about a 1.0-fold increase to about a 4.0-fold increase, about a 1.0-fold increase to about a 3.5-fold increase, about 1.0-fold increase to about a 3.0-fold increase, about a 1.0-fold increase to about a 2.8-fold increase, about a 1.0-fold increase to about a 2.6-fold increase, about a 1.0-fold increase to about a 2.5-fold increase, about a 1.0-fold increase to about a 2.4-fold increase, about a 1.0-fold increase to about a 2.2-fold increase, about a 1.0-fold increase to about a 2.0-fold increase, about a 1.0-fold increase to about a 1.8-fold increase, about a 1.0-fold increase to about a 1.6-fold increase, about a 1.0-fold increase to about a 1.5-fold increase, about a 1.0-fold increase to about a 1.4-fold increase, about a 1.0-fold increase to about a 1.2-fold increase, about a 1.2-fold increase to about a 100-fold increase, about 1.2-fold increase to about a 90-fold increase, about 1.2-fold increase to about a 80-fold increase, about a 1.2-fold increase to about a 70-fold increase, about a 1.2-fold increase to about a 60-fold increase, about a 1.2-fold increase to about a 50-fold increase, about a 1.2-fold increase to about a 40-fold increase, about a 1.2-fold increase to about a 30-fold increase, about 1.2-fold increase to about 20-fold increase, about a 1.2-fold increase to about a 10-fold increase, about a 1.2-fold increase to about a 9.5-fold increase, about a 1.2-fold increase to about a 9.0-fold increase, about a 1.2-fold increase to about a 8.5-fold increase, about a 1.2-fold increase to about a 8.0-fold increase, about a 1.2-fold increase to about a 7.5-fold increase, about a 1.2-fold increase to about a 7.0-fold increase, about a 1.2-fold increase to about a 6.5-fold increase, about a 1.2-fold increase to about a 6.0-fold increase, about a 1.2-fold increase to about a 5.5-fold increase, about a 1.2-fold increase to about a 5.0-fold increase, about a 1.2-fold increase to about a 4.5-fold increase, about a 1.2-fold increase to about a 4.0-fold increase, about a 1.2-fold increase to about a 3.5-fold increase, about 1.2-fold increase to about a 3.0-fold increase, about a 1.2-fold increase to about a 2.8-fold increase, about a 1.2-fold increase to about a 2.6-fold increase, about a 1.2-fold increase to about a 2.5-fold increase, about a 1.2-fold increase to about a 2.4-fold increase, about a 1.2-fold increase to about a 2.2-fold increase, about a 1.2-fold increase to about a 2.0-fold increase, about a 1.2-fold increase to about a 1.8-fold increase, about a 1.2-fold increase to about a 1.6-fold increase, about a 1.2-fold increase to about a 1.5-fold increase, about a 1.2-fold increase to about a 1.4-fold increase, about a 1.4-fold increase to about a 100-fold increase, about 1.4-fold increase to about a 90-fold increase, about 1.4-fold increase to about a 80-fold increase, about a 1.4-fold increase to about a 70-fold increase, about a 1.4-fold increase to about a 60-fold increase, about a 1.4-fold increase to about a 50-fold increase, about a 1.4-fold increase to about a 40-fold increase, about a 1.4-fold increase to about a 30-fold increase, about 1.4-fold increase to about 20-fold increase, about a 1.4-fold increase to about a 10-fold increase, about a 1.4-fold increase to about a 9.5-fold increase, about a 1.4-fold increase to about a 9.0-fold increase, about a 1.4-fold increase to about a 8.5-fold increase, about a 1.4-fold increase to about a 8.0-fold increase, about a 1.4-fold increase to about a 7.5-fold increase, about a 1.4-fold increase to about a 7.0-fold increase, about a 1.4-fold increase to about a 6.5-fold increase, about a 1.4-fold increase to about a 6.0-fold increase, about a 1.4-fold increase to about a 5.5-fold increase, about a 1.4-fold increase to about a 5.0-fold increase, about a 1.4-fold increase to about a 4.5-fold increase, about a 1.4-fold increase to about a 4.0-fold increase, about a 1.4-fold increase to about a 3.5-fold increase, about 1.4-fold increase to about a 3.0-fold increase, about a 1.4-fold increase to about a 2.8-fold increase, about a 1.4-fold increase to about a 2.6-fold increase, about a 1.4-fold increase to about a 2.5-fold increase, about a 1.4-fold increase to about a 2.4-fold increase, about a 1.4-fold increase to about a 2.2-fold increase, about a 1.4-fold increase to about a 2.0-fold increase, about a 1.4-fold increase to about a 1.8-fold increase, about a 1.4-fold increase to about a 1.6-fold increase, about a 1.6-fold increase to about a 10-fold increase, about a 1.6-fold increase to about a 100-fold increase, about 1.6-fold increase to about a 90-fold increase, about 1.6-fold increase to about a 80-fold increase, about a 1.6-fold increase to about a 70-fold increase, about a 1.6-fold increase to about a 60-fold increase, about a 1.6-fold increase to about a 50-fold increase, about a 1.6-fold increase to about a 40-fold increase, about a 1.6-fold increase to about a 30-fold increase, about 1.6-fold increase to about 20-fold increase, about a 1.6-fold increase to about a 9.5-fold increase, about a 1.6-fold increase to about a 9.0-fold increase, about a 1.6-fold increase to about a 8.5-fold increase, about a 1.6-fold increase to about a 8.0-fold increase, about a 1.6-fold increase to about a 7.5-fold increase, about a 1.6-fold increase to about a 7.0-fold increase, about a 1.6-fold increase to about a 6.5-fold increase, about a 1.6-fold increase to about a 6.0-fold increase, about a 1.6-fold increase to about a 5.5-fold increase, about a 1.6-fold increase to about a 5.0-fold increase, about a 1.6-fold increase to about a 4.5-fold increase, about a 1.6-fold increase to about a 4.0-fold increase, about a 1.6-fold increase to about a 3.5-fold increase, about 1.6-fold increase to about a 3.0-fold increase, about a 1.6-fold increase to about a 2.8-fold increase, about a 1.6-fold increase to about a 2.6-fold increase, about a 1.6-fold increase to about a 2.5-fold increase, about a 1.6-fold increase to about a 2.4-fold increase, about a 1.6-fold increase to about a 2.2-fold increase, about a 1.6-fold increase to about a 2.0-fold increase, about a 1.6-fold increase to about a 1.8-fold increase, about a 1.8-fold increase to about a 100-fold increase, about 1.8-fold increase to about a 90-fold increase, about 1.8-fold increase to about a 80-fold increase, about a 1.8-fold increase to about a 70-fold increase, about a 1.8-fold increase to about a 60-fold increase, about a 1.8-fold increase to about a 50-fold increase, about a 1.8-fold increase to about a 40-fold increase, about a 1.8-fold increase to about a 30-fold increase, about 1.8-fold increase to about 20-fold increase, about a 1.8-fold increase to about a 10-fold increase, about a 1.8-fold increase to about a 9.5-fold increase, about a 1.8-fold increase to about a 9.0-fold increase, about a 1.8-fold increase to about a 8.5-fold increase, about a 1.8-fold increase to about a 8.0-fold increase, about a 1.8-fold increase to about a 7.5-fold increase, about a 1.8-fold increase to about a 7.0-fold increase, about a 1.8-fold increase to about a 6.5-fold increase, about a 1.8-fold increase to about a 6.0-fold increase, about a 1.8-fold increase to about a 5.5-fold increase, about a 1.8-fold increase to about a 5.0-fold increase, about a 1.8-fold increase to about a 4.5-fold increase, about a 1.8-fold increase to about a 4.0-fold increase, about a 1.8-fold increase to about a 3.5-fold increase, about 1.8-fold increase to about a 3.0-fold increase, about a 1.8-fold increase to about a 2.8-fold increase, about a 1.8-fold increase to about a 2.6-fold increase, about a 1.8-fold increase to about a 2.5-fold increase, about a 1.8-fold increase to about a 2.4-fold increase, about a 1.8-fold increase to about a 2.2-fold increase, about a 1.8-fold increase to about a 2.0-fold increase, about a 2.0-fold increase to about a 100-fold increase, about 2.0-fold increase to about a 90-fold increase, about 2.0-fold increase to about a 80-fold increase, about a 2.0-fold increase to about a 70-fold increase, about a 2.0-fold increase to about a 60-fold increase, about a 2.0-fold increase to about a 50-fold increase, about a 2.0-fold increase to about a 40-fold increase, about a 2.0-fold increase to about a 30-fold increase, about 2.0-fold increase to about 20-fold increase, about a 2.0-fold increase to about a 10-fold increase, about a 2.0-fold increase to about a 9.5-fold increase, about a 2.0-fold increase to about a 9.0-fold increase, about a 2.0-fold increase to about a 8.5-fold increase, about a 2.0-fold increase to about a 8.0-fold increase, about a 2.0-fold increase to about a 7.5-fold increase, about a 2.0-fold increase to about a 7.0-fold increase, about a 2.0-fold increase to about a 6.5-fold increase, about a 2.0-fold increase to about a 6.0-fold increase, about a 2.0-fold increase to about a 5.5-fold increase, about a 2.0-fold increase to about a 5.0-fold increase, about a 2.0-fold increase to about a 4.5-fold increase, about a 2.0-fold increase to about a 4.0-fold increase, about a 2.0-fold increase to about a 3.5-fold increase, about 2.0-fold increase to about a 3.0-fold increase, about a 2.0-fold increase to about a 2.8-fold increase, about a 2.0-fold increase to about a 2.6-fold increase, about a 2.0-fold increase to about a 2.5-fold increase, about a 2.0-fold increase to about a 2.4-fold increase, about a 2.0-fold increase to about a 2.2-fold increase, about a 2.2-fold increase to about a 100-fold increase, about 2.2-fold increase to about a 90-fold increase, about 2.2-fold increase to about a 80-fold increase, about a 2.2-fold increase to about a 70-fold increase, about a 2.2-fold increase to about a 60-fold increase, about a 2.2-fold increase to about a 50-fold increase, about a 2.2-fold increase to about a 40-fold increase, about a 2.2-fold increase to about a 30-fold increase, about 2.2-fold increase to about 20-fold increase, about a 2.2-fold increase to about a 10-fold increase, about a 2.2-fold increase to about a 9.5-fold increase, about a 2.2-fold increase to about a 9.0-fold increase, about a 2.2-fold increase to about a 8.5-fold increase, about a 2.2-fold increase to about a 8.0-fold increase, about a 2.2-fold increase to about a 7.5-fold increase, about a 2.2-fold increase to about a 7.0-fold increase, about a 2.2-fold increase to about a 6.5-fold increase, about a 2.2-fold increase to about a 6.0-fold increase, about a 2.2-fold increase to about a 5.5-fold increase, about a 2.2-fold increase to about a 5.0-fold increase, about a 2.2-fold increase to about a 4.5-fold increase, about a 2.2-fold increase to about a 4.0-fold increase, about a 2.2-fold increase to about a 3.5-fold increase, about 2.2-fold increase to about a 3.0-fold increase, about a 2.2-fold increase to about a 2.8-fold increase, about a 2.2-fold increase to about a 2.6-fold increase, about a 2.2-fold increase to about a 2.5-fold increase, about a 2.2-fold increase to about a 2.4-fold increase, about a 2.4-fold increase to about a 100-fold increase, about 2.4-fold increase to about a 90-fold increase, about 2.4-fold increase to about a 80-fold increase, about a 2.4-fold increase to about a 70-fold increase, about a 2.4-fold increase to about a 60-fold increase, about a 2.4-fold increase to about a 50-fold increase, about a 2.4-fold increase to about a 40-fold increase, about a 2.4-fold increase to about a 30-fold increase, about 2.4-fold increase to about 20-fold increase, about a 2.4-fold increase to about a 10-fold increase, about a 2.4-fold increase to about a 9.5-fold increase, about a 2.4-fold increase to about a 9.0-fold increase, about a 2.4-fold increase to about a 8.5-fold increase, about a 2.4-fold increase to about a 8.0-fold increase, about a 2.4-fold increase to about a 7.5-fold increase, about a 2.4-fold increase to about a 7.0-fold increase, about a 2.4-fold increase to about a 6.5-fold increase, about a 2.4-fold increase to about a 6.0-fold increase, about a 2.4-fold increase to about a 5.5-fold increase, about a 2.4-fold increase to about a 5.0-fold increase, about a 2.4-fold increase to about a 4.5-fold increase, about a 2.4-fold increase to about a 4.0-fold increase, about a 2.4-fold increase to about a 3.5-fold increase, about 2.4-fold increase to about a 3.0-fold increase, about a 2.4-fold increase to about a 2.8-fold increase, about a 2.4-fold increase to about a 2.6-fold increase, about a 2.6-fold increase to about a 100-fold increase, about 2.6-fold increase to about a 90-fold increase, about 2.6-fold increase to about a 80-fold increase, about a 2.6-fold increase to about a 70-fold increase, about a 2.6-fold increase to about a 60-fold increase, about a 2.6-fold increase to about a 50-fold increase, about a 2.6-fold increase to about a 40-fold increase, about a 2.6-fold increase to about a 30-fold increase, about 2.6-fold increase to about 20-fold increase, about a 2.6-fold increase to about a 10-fold increase, about a 2.6-fold increase to about a 9.5-fold increase, about a 2.6-fold increase to about a 9.0-fold increase, about a 2.6-fold increase to about a 8.5-fold increase, about a 2.6-fold increase to about a 8.0-fold increase, about a 2.6-fold increase to about a 7.5-fold increase, about a 2.6-fold increase to about a 7.0-fold increase, about a 2.6-fold increase to about a 6.5-fold increase, about a 2.6-fold increase to about a 6.0-fold increase, about a 2.6-fold increase to about a 5.5-fold increase, about a 2.6-fold increase to about a 5.0-fold increase, about a 2.6-fold increase to about a 4.5-fold increase, about a 2.6-fold increase to about a 4.0-fold increase, about a 2.6-fold increase to about a 3.5-fold increase, about 2.6-fold increase to about a 3.0-fold increase, about a 2.6-fold increase to about a 2.8-fold increase, about a 2.8-fold increase to about a 100-fold increase, about 2.8-fold increase to about a 90-fold increase, about 2.8-fold increase to about a 80-fold increase, about a 2.8-fold increase to about a 70-fold increase, about a 2.8-fold increase to about a 60-fold increase, about a 2.8-fold increase to about a 50-fold increase, about a 2.8-fold increase to about a 40-fold increase, about a 2.8-fold increase to about a 30-fold increase, about 2.8-fold increase to about 20-fold increase, about a 2.8-fold increase to about a 10-fold increase, about a 2.8-fold increase to about a 9.5-fold increase, about a 2.8-fold increase to about a 9.0-fold increase, about a 2.8-fold increase to about a 8.5-fold increase, about a 2.8-fold increase to about a 8.0-fold increase, about a 2.8-fold increase to about a 7.5-fold increase, about a 2.8-fold increase to about a 7.0-fold increase, about a 2.8-fold increase to about a 6.5-fold increase, about a 2.8-fold increase to about a 6.0-fold increase, about a 2.8-fold increase to about a 5.5-fold increase, about a 2.8-fold increase to about a 5.0-fold increase, about a 2.8-fold increase to about a 4.5-fold increase, about a 2.8-fold increase to about a 4.0-fold increase, about a 2.8-fold increase to about a 3.5-fold increase, about 2.8-fold increase to about a 3.0-fold increase, about a 3.0-fold increase to about a 100-fold increase, about 3.0-fold increase to about a 90-fold increase, about 3.0-fold increase to about a 80-fold increase, about a 3.0-fold increase to about a 70-fold increase, about a 3.0-fold increase to about a 60-fold increase, about a 3.0-fold increase to about a 50-fold increase, about a 3.0-fold increase to about a 40-fold increase, about a 3.0-fold increase to about a 30-fold increase, about 3.0-fold increase to about 20-fold increase, about a 3.0-fold increase to about a 10-fold increase, about a 3.0-fold increase to about a 9.5-fold increase, about a 3.0-fold increase to about a 9.0-fold increase, about a 3.0-fold increase to about a 8.5-fold increase, about a 3.0-fold increase to about a 8.0-fold increase, about a 3.0-fold increase to about a 7.5-fold increase, about a 3.0-fold increase to about a 7.0-fold increase, about a 3.0-fold increase to about a 6.5-fold increase, about a 3.0-fold increase to about a 6.0-fold increase, about a 3.0-fold increase to about a 5.5-fold increase, about a 3.0-fold increase to about a 5.0-fold increase, about a 3.0-fold increase to about a 4.5-fold increase, about a 3.0-fold increase to about a 4.0-fold increase, about a 3.0-fold increase to about a 3.5-fold increase, about a 3.5-fold increase to about a 100-fold increase, about 3.5-fold increase to about a 90-fold increase, about 3.5-fold increase to about a 80-fold increase, about a 3.5-fold increase to about a 70-fold increase, about a 3.5-fold increase to about a 60-fold increase, about a 3.5-fold increase to about a 50-fold increase, about a 3.5-fold increase to about a 40-fold increase, about a 3.5-fold increase to about a 30-fold increase, about 3.5-fold increase to about 20-fold increase, about a 3.5-fold increase to about a 10-fold increase, about a 3.5-fold increase to about a 9.5-fold increase, about a 3.5-fold increase to about a 9.0-fold increase, about a 3.5-fold increase to about a 8.5-fold increase, about a 3.5-fold increase to about a 8.0-fold increase, about a 3.5-fold increase to about a 7.5-fold increase, about a 3.5-fold increase to about a 7.0-fold increase, about a 3.5-fold increase to about a 6.5-fold increase, about a 3.5-fold increase to about a 6.0-fold increase, about a 3.5-fold increase to about a 5.5-fold increase, about a 3.5-fold increase to about a 5.0-fold increase, about a 3.5-fold increase to about a 4.5-fold increase, about a 3.5-fold increase to about a 4.0-fold increase, about a 4.0-fold increase to about a 100-fold increase, about 4.0-fold increase to about a 90-fold increase, about 4.0-fold increase to about a 80-fold increase, about a 4.0-fold increase to about a 70-fold increase, about a 4.0-fold increase to about a 60-fold increase, about a 4.0-fold increase to about a 50-fold increase, about a 4.0-fold increase to about a 40-fold increase, about a 4.0-fold increase to about a 30-fold increase, about 4.0-fold increase to about 20-fold increase, about a 4.0-fold increase to about a 10-fold increase, about a 4.0-fold increase to about a 9.5-fold increase, about a 4.0-fold increase to about a 9.0-fold increase, about a 4.0-fold increase to about a 8.5-fold increase, about a 4.0-fold increase to about a 8.0-fold increase, about a 4.0-fold increase to about a 7.5-fold increase, about a 4.0-fold increase to about a 7.0-fold increase, about a 4.0-fold increase to about a 6.5-fold increase, about a 4.0-fold increase to about a 6.0-fold increase, about a 4.0-fold increase to about a 5.5-fold increase, about a 4.0-fold increase to about a 5.0-fold increase, about a 4.0-fold increase to about a 4.5-fold increase, about a 4.5-fold increase to about a 100-fold increase, about 4.5-fold increase to about a 90-fold increase, about 4.5-fold increase to about a 80-fold increase, about a 4.5-fold increase to about a 70-fold increase, about a 4.5-fold increase to about a 60-fold increase, about a 4.5-fold increase to about a 50-fold increase, about a 4.5-fold increase to about a 40-fold increase, about a 4.5-fold increase to about a 30-fold increase, about 4.5-fold increase to about 20-fold increase, about a 4.5-fold increase to about a 10-fold increase, about a 4.5-fold increase to about a 9.5-fold increase, about a 4.5-fold increase to about a 9.0-fold increase, about a 4.5-fold increase to about a 8.5-fold increase, about a 4.5-fold increase to about a 8.0-fold increase, about a 4.5-fold increase to about a 7.5-fold increase, about a 4.5-fold increase to about a 7.0-fold increase, about a 4.5-fold increase to about a 6.5-fold increase, about a 4.5-fold increase to about a 6.0-fold increase, about a 4.5-fold increase to about a 5.5-fold increase, about a 4.5-fold increase to about a 5.0-fold increase, about a 5.0-fold increase to about a 100-fold increase, about 5.0-fold increase to about a 90-fold increase, about 5.0-fold increase to about a 80-fold increase, about a 5.0-fold increase to about a 70-fold increase, about a 5.0-fold increase to about a 60-fold increase, about a 5.0-fold increase to about a 50-fold increase, about a 5.0-fold increase to about a 40-fold increase, about a 5.0-fold increase to about a 30-fold increase, about 5.0-fold increase to about 20-fold increase, about a 5.0-fold increase to about a 10-fold increase, about a 5.0-fold increase to about a 9.5-fold increase, about a 5.0-fold increase to about a 9.0-fold increase, about a 5.0-fold increase to about a 8.5-fold increase, about a 5.0-fold increase to about a 8.0-fold increase, about a 5.0-fold increase to about a 7.5-fold increase, about a 5.0-fold increase to about a 7.0-fold increase, about a 5.0-fold increase to about a 6.5-fold increase, about a 5.0-fold increase to about a 6.0-fold increase, about a 5.0-fold increase to about a 5.5-fold increase, about a 5.5-fold increase to about a 100-fold increase, about 5.5-fold increase to about a 90-fold increase, about 5.5-fold increase to about a 80-fold increase, about a 5.5-fold increase to about a 70-fold increase, about a 5.5-fold increase to about a 60-fold increase, about a 5.5-fold increase to about a 50-fold increase, about a 5.5-fold increase to about a 40-fold increase, about a 5.5-fold increase to about a 30-fold increase, about 5.5-fold increase to about 20-fold increase, about a 5.5-fold increase to about a 10-fold increase, about a 5.5-fold increase to about a 9.5-fold increase, about a 5.5-fold increase to about a 9.0-fold increase, about a 5.5-fold increase to about a 8.5-fold increase, about a 5.5-fold increase to about a 8.0-fold increase, about a 5.5-fold increase to about a 7.5-fold increase, about a 5.5-fold increase to about a 7.0-fold increase, about a 5.5-fold increase to about a 6.5-fold increase, about a 5.5-fold increase to about a 6.0-fold increase, about a 6.0-fold increase to about a 100-fold increase, about 6.0-fold increase to about a 90-fold increase, about 6.0-fold increase to about a 80-fold increase, about a 6.0-fold increase to about a 70-fold increase, about a 6.0-fold increase to about a 60-fold increase, about a 6.0-fold increase to about a 50-fold increase, about a 6.0-fold increase to about a 40-fold increase, about a 6.0-fold increase to about a 30-fold increase, about 6.0-fold increase to about 20-fold increase, about a 6.0-fold increase to about a 10-fold increase, about a 6.0-fold increase to about a 9.5-fold increase, about a 6.0-fold increase to about a 9.0-fold increase, about a 6.0-fold increase to about a 8.5-fold increase, about a 6.0-fold increase to about a 8.0-fold increase, about a 6.0-fold increase to about a 7.5-fold increase, about a 6.0-fold increase to about a 7.0-fold increase, about a 6.0-fold increase to about a 6.5-fold increase, about a 6.5-fold increase to about a 100-fold increase, about 6.5-fold increase to about a 90-fold increase, about 6.5-fold increase to about a 80-fold increase, about a 6.5-fold increase to about a 70-fold increase, about a 6.5-fold increase to about a 60-fold increase, about a 6.5-fold increase to about a 50-fold increase, about a 6.5-fold increase to about a 40-fold increase, about a 6.5-fold increase to about a 30-fold increase, about 6.5-fold increase to about 20-fold increase, about a 6.5-fold increase to about a 10-fold increase, about a 6.5-fold increase to about a 9.5-fold increase, about a 6.5-fold increase to about a 9.0-fold increase, about a 6.5-fold increase to about a 8.5-fold increase, about a 6.5-fold increase to about a 8.0-fold increase, about a 6.5-fold increase to about a 7.5-fold increase, about a 6.5-fold increase to about a 7.0-fold increase, about a 7.0-fold increase to about a 100-fold increase, about 7.0-fold increase to about a 90-fold increase, about 7.0-fold increase to about a 80-fold increase, about a 7.0-fold increase to about a 70-fold increase, about a 7.0-fold increase to about a 60-fold increase, about a 7.0-fold increase to about a 50-fold increase, about a 7.0-fold increase to about a 40-fold increase, about a 7.0-fold increase to about a 30-fold increase, about 7.0-fold increase to about 20-fold increase, about a 7.0-fold increase to about a 10-fold increase, about a 7.0-fold increase to about a 9.5-fold increase, about a 7.0-fold increase to about a 9.0-fold increase, about a 7.0-fold increase to about a 8.5-fold increase, about a 7.0-fold increase to about a 8.0-fold increase, about a 7.0-fold increase to about a 7.5-fold increase, about a 7.5-fold increase to about a 100-fold increase, about 7.5-fold increase to about a 90-fold increase, about 7.5-fold increase to about a 80-fold increase, about a 7.5-fold increase to about a 70-fold increase, about a 7.5-fold increase to about a 60-fold increase, about a 7.5-fold increase to about a 50-fold increase, about a 7.5-fold increase to about a 40-fold increase, about a 7.5-fold increase to about a 30-fold increase, about 7.5-fold increase to about 20-fold increase, about a 7.5-fold increase to about a 10-fold increase, about a 7.5-fold increase to about a 9.5-fold increase, about a 7.5-fold increase to about a 9.0-fold increase, about a 7.5-fold increase to about a 8.5-fold increase, about a 7.5-fold increase to about a 8.0-fold increase, about a 8.0-fold increase to about a 100-fold increase, about 8.0-fold increase to about a 90-fold increase, about 8.0-fold increase to about a 80-fold increase, about a 8.0-fold increase to about a 70-fold increase, about a 8.0-fold increase to about a 60-fold increase, about a 8.0-fold increase to about a 50-fold increase, about a 8.0-fold increase to about a 40-fold increase, about a 8.0-fold increase to about a 30-fold increase, about 8.0-fold increase to about 20-fold increase, about a 8.0-fold increase to about a 10-fold increase, about a 8.0-fold increase to about a 9.5-fold increase, about a 8.0-fold increase to about a 9.0-fold increase, about a 8.0-fold increase to about a 8.5-fold increase, about a 8.5-fold increase to about a 100-fold increase, about 8.5-fold increase to about a 90-fold increase, about 8.5-fold increase to about a 80-fold increase, about a 8.5-fold increase to about a 70-fold increase, about a 8.5-fold increase to about a 60-fold increase, about a 8.5-fold increase to about a 50-fold increase, about a 8.5-fold increase to about a 40-fold increase, about a 8.5-fold increase to about a 30-fold increase, about 8.5-fold increase to about 20-fold increase, about a 8.5-fold increase to about a 10-fold increase, about a 8.5-fold increase to about a 9.5-fold increase, about a 8.5-fold increase to about a 9.0-fold increase, about a 9.0-fold increase to about a 100-fold increase, about 9.0-fold increase to about a 90-fold increase, about 9.0-fold increase to about a 80-fold increase, about a 9.0-fold increase to about a 70-fold increase, about a 9.0-fold increase to about a 60-fold increase, about a 9.0-fold increase to about a 50-fold increase, about a 9.0-fold increase to about a 40-fold increase, about a 9.0-fold increase to about a 30-fold increase, about 9.0-fold increase to about 20-fold increase, about a 9.0-fold increase to about a 10-fold increase, about a 9.0-fold increase to about a 9.5-fold increase, about a 9.5-fold increase to about a 100-fold increase, about 9.5-fold increase to about a 90-fold increase, about 9.5-fold increase to about a 80-fold increase, about a 9.5-fold increase to about a 70-fold increase, about a 9.5-fold increase to about a 60-fold increase, about a 9.5-fold increase to about a 50-fold increase, about a 9.5-fold increase to about a 40-fold increase, about a 9.5-fold increase to about a 30-fold increase, about 9.5-fold increase to about 20-fold increase, about a 9.5-fold increase to about a 10-fold increase, about a 10-fold increase to about a 100-fold increase, about 10-fold increase to about a 90-fold increase, about 10-fold increase to about a 80-fold increase, about a 10-fold increase to about a 70-fold increase, about a 10-fold increase to about a 60-fold increase, about a 10-fold increase to about a 50-fold increase, about a 10-fold increase to about a 40-fold increase, about a 10-fold increase to about a 30-fold increase, about 10-fold increase to about 20-fold increase, about a 20-fold increase to about a 100-fold increase, about 20-fold increase to about a 90-fold increase, about 20-fold increase to about a 80-fold increase, about a 20-fold increase to about a 70-fold increase, about a 20-fold increase to about a 60-fold increase, about a 20-fold increase to about a 50-fold increase, about a 20-fold increase to about a 40-fold increase, about a 20-fold increase to about a 30-fold increase, about a 30-fold increase to about a 100-fold increase, about 30-fold increase to about a 90-fold increase, about 30-fold increase to about a 80-fold increase, about a 30-fold increase to about a 70-fold increase, about a 30-fold increase to about a 60-fold increase, about a 30-fold increase to about a 50-fold increase, about a 30-fold increase to about a 40-fold increase, about a 40-fold increase to about a 100-fold increase, about 40-fold increase to about a 90-fold increase, about 40-fold increase to about a 80-fold increase, about a 40-fold increase to about a 70-fold increase, about a 40-fold increase to about a 60-fold increase, about a 40-fold increase to about a 50-fold increase, about a 50-fold increase to about a 100-fold increase, about 50-fold increase to about a 90-fold increase, about 50-fold increase to about a 80-fold increase, about a 50-fold increase to about a 70-fold increase, about a 50-fold increase to about a 60-fold increase, about a 60-fold increase to about a 100-fold increase, about 60-fold increase to about a 90-fold increase, about 60-fold increase to about a 80-fold increase, about a 60-fold increase to about a 70-fold increase, about a 70-fold increase to about a 100-fold increase, about 70-fold increase to about a 90-fold increase, about 70-fold increase to about a 80-fold increase, about a 80-fold increase to about a 100-fold increase, about 80-fold increase to about a 90-fold increase, or about a 90-fold increase to about a 100-fold increase) in toxin liberation in the target mammalian cell (e.g., any of the target mammalian cells described herein) as compared to a composition including the same amount of a control ABPC (e.g., any of the exemplary control ABPCs described herein).

In some examples of any of the ABPCs described herein, a composition including the ABPC (e.g., any of the ABPCs described herein) can provide for an increase (e.g., a detectable increase) (e.g., at least a 1% increase, at least a 2% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 120% increase, at least a 140% increase, at least a 160% increase, at least a 180% increase, at least a 200% increase, at least a 250% increase, at least a 300% increase, at least a 350% increase, at least a 400% increase, at least a 450% increase, at least a 500% increase, at least a 1,000% increase, at least a 2,000% increase, at least a 3,000% increase, at least a 4,000% increase, at least a 5,000% increase, at least a 6,000% increase, at least a 7,000% increase, at least a 8,000% increase, at a least a 9,000% increase, or at least a 10,000% increase, or about a 1% increase to about a 10,000% increase (e.g., or any of the subranges of this range described herein)) in target mammalian cell killing (e.g., any of the exemplary target mammalian cells described herein) as compared to a composition including the same amount of a control ABPC (e.g., any of the exemplary control ABPCs described herein).

In some examples of any of the ABPCs described herein, a composition including the ABPC (e.g., any of the ABPCs described herein) can provide for an increase (e.g., a detectable increase) (e.g., at least a 0.1-fold increase, at least a 0.2-fold increase, at least a 0.3-fold increase, at least a 0.4-fold increase, at least a 0.5-fold increase, at least a 0.6-fold increase, at least a 0.7-fold increase, at least a 0.8-fold increase, at least a 0.9-fold increase, at least a 1.0-fold increase, at least a 1.2-fold increase, at least a 1.4-fold increase, at least a 1.5-fold increase, at least a 1.6-fold increase, at least a 1.8-fold increase, at least a 2.0-fold increase, at least a 2.2-fold increase, at least a 2.4-fold increase, at least a 2.5-fold increase, at least a 2.6-fold increase, at least a 2.8-fold increase, at least a 3.0-fold increase, at least a 3.5-fold increase, at least a 4.0-fold increase, at least a 4.5-fold increase, at least a 5.0-fold increase, at least a 5.5-fold increase, at least a 6.0-fold increase, at least a 6.5-fold increase, at least a 7.0-fold increase, at least a 7.5-fold increase, at least a 8.0-fold increase, at least a 8.5-fold increase, at least a 9.0-fold increase, at least a 9.5-fold increase, at least a 10-fold increase, at least a 15-fold increase, at least a 20-fold increase, at least a 25-fold increase, at least a 30-fold increase, at least a 35-fold increase, at least a 40-fold increase, at least a 40-fold increase, at least a 45-fold increase, at least a 50-fold increase, at least a 55-fold increase, at least a 60-fold increase, at least a 65-fold increase, at least a 70-fold increase, at least a 80-fold increase, at least a 85-fold increase, at least a 90-fold increase, at least a 95-fold increase, or at least a 100-fold increase, or about a 0.1-fold increase to about a 100-fold increase (or any of the subranges of this range described herein)) in target mammalian cell killing (e.g., any of the exemplary target mammalian cells described herein) as compared to a composition including the same amount of a control ABPC (e.g., any of the exemplary control ABPCs described herein).

In some examples of any of the ABPCs described herein, a composition including any of the ABPCs described herein (e.g., upon contacting target mammalian cells presenting DLL3 on their surface) results in decreased (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, about a 1% decrease to about a 99% decrease, or any of the subranges of this range described herein) IC₅₀ (for target mammalian cell killing) as compared to the IC50 for a composition including the same amount of a control ABPC (e.g., any of the control ABPCs described herein) (e.g., upon contacting the same target mammalian cells).

In some examples of any of the ABPCs described herein, a composition including any of the ABPCs described herein (e.g., upon contacting target mammalian cells presenting DLL3 on their surface) can provide for an increase (e.g., at least a 0.1-fold increase, at least a 0.2-fold increase, at least a 0.4-fold increase, at least a 0.6-fold increase, at least a 0.8-fold increase, at least a 1-fold increase, at least a 2-fold increase, at least a 5-fold increase, at least a 10-fold increase, at least a 15-fold increase, at least a 20-fold increase, at least a 25-fold increase, at least a 30-fold increase, at least a 35-fold increase, at least a 40-fold increase, at least a 45-fold increase, at least a 50-fold increase, at least a 55-fold increase, at least a 60-fold increase, at least a 65-fold increase, at least a 70-fold increase, at least a 75-fold increase, at least a 80-fold increase, at least a 85-fold increase, at least a 90-fold increase, at least a 95-fold increase, or at least a 100-fold increase, or about a 0.1-fold increase to about 500-fold increase (or any of the subranges of this range described herein) in the ratio of K_{D} on target mammalian cells presenting DLL3 on their surface at a neutral pH (a pH of about 7.0 to about 8.0) to IC50 at the neutral pH on the same target cells, e.g., as compared to a control ABPC (e.g., any of the exemplary control ABPCs described herein).

In some examples of any of the ABPCs described herein, a composition including the ABPC (e.g., any of the ABPCs described herein) can provide for an increase (e.g., a detectable increase) (e.g., at least a 1% increase, at least a 2% increase, at least a 5% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 120% increase, at least a 140% increase, at least a 160% increase, at least a 180% increase, at least a 200% increase, at least a 250% increase, at least a 300% increase, at least a 350% increase, at least a 400% increase, at least a 450% increase, at least a 500% increase, at least a 1,000% increase, at least a 2,000% increase, at least a 3,000% increase, at least a 4,000% increase, at least a 5,000% increase, at least a 6,000% increase, at least a 7,000% increase, at least a 8,000% increase, at least a 9,000% increase, or at least a 10,000% increase, or about a 1% increase to about a 10,000% increase (e.g., or any of the subranges of this range described herein)) in endolysosomal delivery in the target mammalian cell (e.g., any of the exemplary target mammalian cells described herein) as compared to a composition including the same amount of a control ABPC (e.g., any of the exemplary control ABPCs described herein).

In some examples of any of the ABPCs described herein, a composition including the ABPC (e.g., any of the ABPCs described herein) can provide for an increase (e.g., a detectable increase) (e.g., at least a 0.1-fold increase, at least a 0.2-fold increase, at least a 0.3-fold increase, at least a 0.4-fold increase, at least a 0.5-fold increase, at least a 0.6-fold increase, at least a 0.7-fold increase, at least a 0.8-fold increase, at least a 0.9-fold increase, at least a 1.0-fold increase, at least a 1.2-fold increase, at least a 1.4-fold increase, at least a 1.5-fold increase, at least a 1.6-fold increase, at least a 1.8-fold increase, at least a 2.0-fold increase, at least a 2.2-fold increase, at least a 2.4-fold increase, at least a 2.5-fold increase, at least a 2.6-fold increase, at least a 2.8-fold increase, at least a 3.0-fold increase, at least a 3.5-fold increase, at least a 4.0-fold increase, at least a 4.5-fold increase, at least a 5.0-fold increase, at least a 5.5-fold increase, at least a 6.0-fold increase, at least a 6.5-fold increase, at least a 7.0-fold increase, at least a 7.5-fold increase, at least a 8.0-fold increase, at least a 8.5-fold increase, at least a 9.0-fold increase, at least a 9.5-fold increase, at least a 10-fold increase, at least a 15-fold increase, at least a 20-fold increase, at least a 25-fold increase, at least a 30-fold increase, at least a 35-fold increase, at least a 40-fold increase, at least a 45-fold increase, at least a 50-fold increase, at least a 55-fold increase, at least a 60-fold increase, at least a 65-fold increase, at least a 70-fold increase, at least a 75-fold increase, at least a 80-fold increase, at least a 85-fold increase, at least a 90-fold increase, at least a 95-fold increase, or at least a 100-fold increase, or about a 0.1-fold increase to about a 100-fold increase (or any of the subranges of this range described herein)) in endolysosomal delivery in the target mammalian cell (e.g., any of the exemplary target mammalian cells described herein) as compared to a composition including the same amount of a control ABPC (e.g., any of the exemplary control ABPCs described herein).

In examples of any of the ABPCs described herein, the target mammalian cell does not express an FcRn receptor, or expresses a lower (e.g., a detectably lower) level (e.g., at least a 1% decreased, at least a 2% decreased, at least a 5% decreased, at least a 10% decrease, at least a 15% decreased, at least a 20% decreased, at least a 25% decreased, at least a 30% decreased, at least a 35% decreased, at least a 40% decreased, at least a 45% decreased, at least a 50% decreased, at least a 55% decreased, at least a 60% decreased, at least a 65% decreased, at least a 70% decreased, at least a 75% decreased, at least a 80% decreased, at least a 85% decreased, at least a 90% decreased, at least a 95% decreased, or at least a 99% decreased level) of FcRn receptor as compared to a FcRn expressing control cell (e.g., HUVEC—ThermoFisher #C0035C). In some examples of any of the ABPCs described herein, the target mammalian cell is a cancer cell. In some examples of any of the ABPCs described herein, the ABPC is cytotoxic or cytostatic to the target mammalian cell.

In some examples of any of the ABPCs described herein, a composition including any of the ABPCs described herein (e.g., upon administration to a subject) results in less (e.g., a 1% decrease to about a 99% decrease, or any of the subranges of this range described herein) of a reduction in the level of DLL3 presented on the surface of the target cell as compared to a composition including the same amount of a control ABPC (e.g., any of the control ABPCs described herein). In some examples of any of the ABPCs described herein, the composition does not result in a detectable reduction in the level of the DLL3 presented on the surface of the target mammalian cell.

In some examples of any of the ABPCs described herein, the ABPC is cross-reactive with a non-human primate DLL3 and a human DLL3. In some examples of any of the ABPCs described herein, the ABPC is cross-reactive with a non-human primate DLL3, a human DLL3, and one or both of rat DLL3 and a mouse DLL3. In some examples of any of the ABPCs described herein, the ABPC is cross-reactive with a non-human primate DLL3, a human DLL3, a rat DLL3, and a mouse DLL3. In some examples of any of the ABPCs described herein, the ABPC is cross-reactive with mouse DLL3 and rat DLL3. In some examples of any of the ABPCs described herein, the antigen-binding domain binds to an epitope of DLL3 that is present on the surface of cells from an Old World Monkey.

Some examples of any of the ABPCs described herein can further include a second antigen-binding domain (e.g., any of the exemplary antigen-binding domains described herein). Non-limiting aspects of these methods are described below, and can be used in any combination without limitation. Additional aspects of these methods are known in the art.

### DLL3 or Epitope of DLL3

Delta-like 3 (DLL3) is a tumor antigen that is known in the art, and is the target of therapeutic antibodies in oncology (Saunders L R et al (2015) “A DLL3-targeted antibody-drug conjugate eradicates high-grade pulmonary neuroendocrine tumor-initiating cells in vivo.” Science Translational Medicine 7(302):302ra136). The sequence of the mature Human DLL3 can be found in SEQ ID NO: 9. The sequence of the cDNA encoding the mature Human DLL3 can be found in SEQ ID NO: 10. The sequence of the extracellular domain of DLL3 can be found in SEQ ID NO: 11. The sequence of the cDNA encoding the extracellular domain of DLL3 can be found in SEQ ID NO: 12.

### Antigen-Binding Protein Constructs

Any of the antigen-binding protein constructs (ABPCs) described herein can be a single polypeptide, or can include two, three, four, five, six, seven, eight, nine, or ten (the same or different) polypeptides. In some embodiments where the ABPC is a single polypeptide, the ABPC can include a single antigen-binding domain or two antigen-binding domains. In some embodiments where the ABPC is a single polypeptide and includes two antigen-binding domains, the first and second antigen-binding domains can be identical or different from each other (and can specifically bind to the same or different antigens or epitopes).

In some embodiments where the ABPC is a single polypeptide, the first antigen-binding domain and the second antigen-binding domain (if present) can each be independently selected from the group of: a VH domain, a VHH domain, a VNAR domain, and a scFv. In some embodiments where the ABPC is a single polypeptide, the antigen-binding protein construct can be a BiTe, a (scFv)2, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the ABPC is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the ABPC is a single polypeptide and includes two antigen-binding domains, the first antigen-binding domain and the second antigen-binding domain can both be VHH domains, or at least one antigen-binding domain can be a VHH domain. In some embodiments where the ABPC is a single polypeptide and includes two antigen-binding domains, the first antigen-binding domain and the second antigen-binding domain are both V_{NAR} domains, or at least one antigen-binding domain is a V_{NAR} domain. In some embodiments where the ABPC is a single polypeptide, the first antigen-binding domain is a scFv domain. In some embodiments where the ABPC is a single polypeptide and includes two antigen-binding domains, the first antigen-binding domain and the second antigen-binding domain can both be scFv domains, or at least one antigen-binding domain can be a scFv domain.

In some embodiments, the ABPC can include two or more polypeptides (e.g., two, three, four, five, six, seven, eight, nine, or ten polypeptides). In some embodiments where the ABPC includes two or more polypeptides, two, three, four, five or six of the polypeptides of the two or more polypeptides can be identical.

In some embodiments where the ABPC includes two or more polypeptides (e.g., two, three, four, five, six, seven, eight, nine, or ten polypeptides), two or more of the polypeptides of the ABPC can assemble (e.g., non-covalently assemble) to form one or more antigen-binding domains, e.g., an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

A “Fv” fragment includes a non-covalently-linked dimer of one heavy chain variable domain and one light chain variable domain.

A “Fab” fragment includes the constant domain of the light chain and the first constant domain (C_{HI}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

Additional aspects of ABPCs are known in the art.

### Antigen-Binding Domains

In some embodiments of any of the antigen-binding protein constructs (ABPCs) described herein, the dissociation rate of the first antigen-binding domain (and optionally the second antigen-binding domain, if present) at a pH of about 4.0 to about 6.5 (e.g., about 4.0 to about 6.4, about 4.0 to about 6.3, about 4.0 to about 6.2, about 4.0 to about 6.1, about 4.0 to about 6.0, about 4.0 to about 5.9, about 4.0 to about 5.8, about 4.0 to about 5.7, about 4.0 to about 5.6, about 4.0 to about 5.5, about 4.0 to about 5.4, about 4.0 to about 5.3, about 4.0 to about 5.2, about 4.0 to about 5.1, about 4.0 to about 5.0, about 4.0 to about 4.9, about 4.0 to about 4.8, about 4.0 to about 4.7, about 4.0 to about 4.6, about 4.0 to about 4.5, about 4.0 to about 4.4, about 4.0 to about 4.3, about 4.0 to about 4.2, about 4.0 to about 4.1, about 4.1 to about 6.5, about 4.1 to about 6.4, about 4.1 to about 6.3, about 4.1 to about 6.2, about 4.1 to about 6.1, about 4.1 to about 6.0, about 4.1 to about 5.9, about 4.1 to about 5.8, about 4.1 to about 5.7, about 4.1 to about 5.6, about 4.1 to about 5.5, about 4.1 to about 5.4, about 4.1 to about 5.3, about 4.1 to about 5.2, about 4.1 to about 5.1, about 4.1 to about 5.0, about 4.1 to about 4.9, about 4.1 to about 4.8, about 4.1 to about 4.7, about 4.1 to about 4.6, about 4.1 to about 4.5, about 4.1 to about 4.4, about 4.1 to about 4.3, about 4.1 to about 4.2, about 4.2 to about 6.5, about 4.2 to about 6.4, about 4.2 to about 6.3, about 4.2 to about 6.2, about 4.2 to about 6.1, about 4.2 to about 6.0, about 4.2 to about 5.9, about 4.2 to about 5.8, about 4.2 to about 5.7, about 4.2 to about 5.6, about 4.2 to about 5.5, about 4.2 to about 5.4, about 4.2 to about 5.3, about 4.2 to about 5.2, about 4.2 to about 5.1, about 4.2 to about 5.0, about 4.2 to about 4.9, about 4.2 to about 4.8, about 4.2 to about 4.7, about 4.2 to about 4.6, about 4.2 to about 4.5, about 4.2 to about 4.4, about 4.2 to about 4.3, about 4.3 to about 6.5, about 4.3 to about 6.4, about 4.3 to about 6.3, about 4.3 to about 6.2, about 4.3 to about 6.1, about 4.3 to about 6.0, about 4.3 to about 5.9, about 4.3 to about 5.8, about 4.3 to about 5.7, about 4.3 to about 5.6, about 4.3 to about 5.5, about 4.3 to about 5.4, about 4.3 to about 5.3, about 4.3 to about 5.2, about 4.3 to about 5.1, about 4.3 to about 5.0, about 4.3 to about 4.9, about 4.3 to about 4.8, about 4.3 to about 4.7, about 4.3 to about 4.6, about 4.3 to about 4.5, about 4.3 to about 4.4, about 4.4 to about 6.5, about 4.4 to about 6.4, about 4.4 to about 6.3, about 4.4 to about 6.2, about 4.4 to about 6.1, about 4.4 to about 6.0, about 4.4 to about 5.9, about 4.4 to about 5.8, about 4.4 to about 5.7, about 4.4 to about 5.6, about 4.4 to about 5.5, about 4.4 to about 5.4, about 4.4 to about 5.3, about 4.4 to about 5.2, about 4.4 to about 5.1, about 4.4 to about 5.0, about 4.4 to about 4.9, about 4.4 to about 4.8, about 4.4 to about 4.7, about 4.4 to about 4.6, about 4.4 to about 4.5, about 4.5 to about 6.5, about 4.5 to about 6.4, about 4.5 to about 6.3, about 4.5 to about 6.2, about 4.5 to about 6.1, about 4.5 to about 6.0, about 4.5 to about 5.9, about 4.5 to about 5.8, about 4.5 to about 5.7, about 4.5 to about 5.6, about 4.5 to about 5.5, about 4.5 to about 5.4, about 4.5 to about 5.3, about 4.5 to about 5.2, about 4.5 to about 5.1, about 4.5 to about 5.0, about 4.5 to about 4.9, about 4.5 to about 4.8, about 4.5 to about 4.7, about 4.5 to about 4.6, about 4.6 to about 6.5, about 4.6 to about 6.4, about 4.6 to about 6.3, about 4.6 to about 6.2, about 4.6 to about 6.1, about 4.6 to about 6.0, about 4.6 to about 5.9, about 4.6 to about 5.8, about 4.6 to about 5.7, about 4.6 to about 5.6, about 4.6 to about 5.5, about 4.6 to about 5.4, about 4.6 to about 5.3, about 4.6 to about 5.2, about 4.6 to about 5.1, about 4.6 to about 5.0, about 4.6 to about 4.9, about 4.6 to about 4.8, about 4.6 to about 4.7, about 4.7 to about 6.5, about 4.7 to about 6.4, about 4.7 to about 6.3, about 4.7 to about 6.2, about 4.7 to about 6.1, about 4.7 to about 6.0, about 4.7 to about 5.9, about 4.7 to about 5.8, about 4.7 to about 5.7, about 4.7 to about 5.6, about 4.7 to about 5.5, about 4.7 to about 5.4, about 4.7 to about 5.3, about 4.7 to about 5.2, about 4.7 to about 5.1, about 4.7 to about 5.0, about 4.7 to about 4.9, about 4.7 to about 4.8, about 4.8 to about 6.5, about 4.8 to about 6.4, about 4.8 to about 6.3, about 4.8 to about 6.2, about 4.8 to about 6.1, about 4.8 to about 6.0, about 4.8 to about 5.9, about 4.8 to about 5.8, about 4.8 to about 5.7, about 4.8 to about 5.6, about 4.8 to about 5.5, about 4.8 to about 5.4, about 4.8 to about 5.3, about 4.8 to about 5.2, about 4.8 to about 5.1, about 4.8 to about 5.0, about 4.8 to about 4.9, about 4.9 to about 6.5, about 4.9 to about 6.4, about 4.9 to about 6.3, about 4.9 to about 6.2, about 4.9 to about 6.1, about 4.9 to about 6.0, about 4.9 to about 5.9, about 4.9 to about 5.8, about 4.9 to about 5.7, about 4.9 to about 5.6, about 4.9 to about 5.5, about 4.9 to about 5.4, about 4.9 to about 5.3, about 4.9 to about 5.2, about 4.9 to about 5.1, about 4.9 to about 5.0, about 5.0 to about 6.5, about 5.0 to about 6.4, about 5.0 to about 6.3, about 5.0 to about 6.2, about 5.0 to about 6.1, about 5.0 to about 6.0, about 5.0 to about 5.9, about 5.0 to about 5.8, about 5.0 to about 5.7, about 5.0 to about 5.6, about 5.0 to about 5.5, about 5.0 to about 5.4, about 5.0 to about 5.3, about 5.0 to about 5.2, about 5.0 to about 5.1, about 5.1 to about 6.5, about 5.1 to about 6.4, about 5.1 to about 6.3, about 5.1 to about 6.2, about 5.1 to about 6.1, about 5.1 to about 6.0, about 5.1 to about 5.9, about 5.1 to about 5.8, about 5.1 to about 5.7, about 5.1 to about 5.6, about 5.1 to about 5.5, about 5.1 to about 5.4, about 5.1 to about 5.3, about 5.1 to about 5.2, about 5.2 to about 6.5, about 5.2 to about 6.4, about 5.2 to about 6.3, about 5.2 to about 6.2, about 5.2 to about 6.1, about 5.2 to about 6.0, about 5.2 to about 5.9, about 5.2 to about 5.8, about 5.2 to about 5.7, about 5.2 to about 5.6, about 5.2 to about 5.5, about 5.2 to about 5.4, about 5.2 to about 5.3, about 5.3 to about 6.5, about 5.3 to about 6.4, about 5.3 to about 6.3, about 5.3 to about 6.2, about 5.3 to about 6.1, about 5.3 to about 6.0, about 5.3 to about 5.9, about 5.3 to about 5.8, about 5.3 to about 5.7, about 5.3 to about 5.6, about 5.3 to about 5.5, about 5.3 to about 5.4, about 5.4 to about 6.5, about 5.4 to about 6.4, about 5.4 to about 6.3, about 5.4 to about 6.2, about 5.4 to about 6.1, about 5.4 to about 6.0, about 5.4 to about 5.9, about 5.4 to about 5.8, about 5.4 to about 5.7, about 5.4 to about 5.6, about 5.4 to about 5.5, about 5.5 to about 6.5, about 5.5 to about 6.4, about 5.5 to about 6.3, about 5.5 to about 6.2, about 5.5 to about 6.1, about 5.5 to about 6.0, about 5.5 to about 5.9, about 5.5 to about 5.8, about 5.5 to about 5.7, about 5.5 to about 5.6, about 5.6 to about 6.5, about 5.6 to about 6.4, about 5.6 to about 6.3, about 5.6 to about 6.2, about 5.6 to about 6.1, about 5.6 to about 6.0, about 5.6 to about 5.9, about 5.6 to about 5.8, about 5.6 to about 5.7, about 5.7 to about 6.5, about 5.7 to about 6.4, about 5.7 to about 6.3, about 5.7 to about 6.2, about 5.7 to about 6.1, about 5.7 to about 6.0, about 5.7 to about 5.9, about 5.7 to about 5.8, about 5.8 to about 6.5, about 5.8 to about 6.4, about 5.8 to about 6.3, about 5.8 to about 6.2, about 5.8 to about 6.1, about 5.8 to about 6.0, about 5.8 to about 5.9, about 5.9 to about 6.5, about 5.9 to about 6.4, about 5.9 to about 6.3, about 5.9 to about 6.2, about 5.9 to about 6.1, about 5.9 to about 6.0, about 6.0 to about 6.5, about 6.0 to about 6.4, about 6.0 to about 6.3, about 6.0 to about 6.2, about 6.0 to about 6.1, about 6.1 to about 6.5, about 6.1 to about 6.4, about 6.1 to about 6.3, about 6.1 to about 6.2, about 6.2 to about 6.5, about 6.2 to about 6.4, about 6.2 to about 6.3, about 6.3 to about 6.5, about 6.3 to about 6.4, or about 6.4 to about 6.5) is faster (e.g., (e.g., at least 5% faster, at least 10% faster, at least 15% faster, at least 20%, at least 25% faster, at least 30% faster, at least 35% faster, at least 40% faster, at least 45% faster, at least 50% faster, at least 55% faster, at least 60% faster, at least 65% faster, at least 70% faster, at least 75% faster, at least 80% faster, at least 85% faster, at least 90% faster, at least 95% faster, at least 100% faster, at least 120% faster, at least 140% faster, at least 160% faster, at least 180% faster, at least 200% faster, at least 220% faster at least 240% faster at least 260% faster at least 280% faster at least 300% faster at least 320% faster at least 340% faster at least 360% faster at least 380% faster at least 400% faster at least 420% faster at least 440% faster at least 460% faster at least 480% faster, at least 500% faster, at least 1,000% faster, at least 2,000% faster, at least 3,000% faster, at least 4,000% faster, at least 5,000%, at least 6,000% faster, at least 7,000% faster, at least 8,000% faster, at least 9,000% faster, or at least 10,000% faster, or about 5% faster to about 10,000% faster, about 5% faster to about 9,000% faster, about 5% faster to about 8,000% faster, about 5% faster to about 7,000% faster, about 5% faster to about 6,000% faster, about 5% faster to about 5,000% faster, about 5% faster to about 4,000% faster, about 5% faster to about 3,000% faster, about 5% faster to about 2,000% faster, about 5% faster to about 1,000% faster, about 5% faster to about 500% faster, about 5% faster to about 480% faster, about 5% faster to about 460% faster, about 5% faster to about 440% faster, about 5% faster to about 420% faster, about 5% faster to about 400% faster, about 5% faster to about 380% faster, about 5% faster to about 360% faster, about 5% faster to about 340% faster, about 5% faster to about 320% faster, about 5% faster to about 300% faster, about 5% faster to about 280% faster, about 5% faster to about 260% faster, about 5% faster to about 240% faster, about 5% faster to about 220% faster, about 5% faster to about 200% faster, about 5% faster to about 180% faster, about 5% faster to about 160% faster, about 5% faster to about 140% faster, about 5% faster to about 120% faster, about 5% faster to about 100% faster, about 5% faster to about 95% faster, about 5% faster to about 90% faster, about 5% faster to about 85% faster, about 5% faster to about 80% faster, about 5% faster to about 75% faster, about 5% faster to about 70% faster, about 5% faster to about 65% faster, about 5% faster to about 60% faster, about 5% faster to about 55% faster, about 5% faster to about 50% faster, about 5% faster to about 45% faster, about 5% faster to about 40% faster, about 5% faster to about 35% faster, about 5% faster to about 30% faster, about 5% faster to about 25% faster, about 5% faster to about 20% faster, about 5% faster to about 15% faster, about 5% faster to about 10% faster, about 10% faster to about 10,000% faster, about 10% faster to about 9,000% faster, about 10% faster to about 8,000% faster, about 10% faster to about 7,000% faster, about 10% faster to about 6,000% faster, about 10% faster to about 5,000% faster, about 10% faster to about 4,000% faster, about 10% faster to about 3,000% faster, about 10% faster to about 2,000% faster, about 10% faster to about 1,000% faster, about 10% faster to about 500% faster, about 10% faster to about 480% faster, about 10% faster to about 460% faster, about 10% faster to about 440% faster, about 10% faster to about 420% faster, about 10% faster to about 400% faster, about 10% faster to about 380% faster, about 10% faster to about 360% faster, about 10% faster to about 340% faster, about 10% faster to about 320% faster, about 10% faster to about 300% faster, about 10% faster to about 280% faster, about 10% faster to about 260% faster, about 10% faster to about 240% faster, about 10% faster to about 220% faster, about 10% faster to about 200% faster, about 10% faster to about 180% faster, about 10% faster to about 160% faster, about 10% faster to about 140% faster, about 10% faster to about 120% faster, about 10% faster to about 100% faster, about 10% faster to about 95% faster, about 10% faster to about 90% faster, about 10% faster to about 85% faster, about 10% faster to about 80% faster, about 10% faster to about 75% faster, about 10% faster to about 70% faster, about 10% faster to about 65% faster, about 10% faster to about 60% faster, about 10% faster to about 55% faster, about 10% faster to about 50% faster, about 10% faster to about 45% faster, about 10% faster to about 40% faster, about 10% faster to about 35% faster, about 10% faster to about 30% faster, about 10% faster to about 25% faster, about 10% faster to about 20% faster, about 10% faster to about 15% faster, about 15% faster to about 10,000% faster, about 15% faster to about 9,000% faster, about 15% faster to about 8,000% faster, about 15% faster to about 7,000% faster, about 15% faster to about 6,000% faster, about 15% faster to about 5,000% faster, about 15% faster to about 4,000% faster, about 15% faster to about 3,000% faster, about 15% faster to about 2,000% faster, about 15% faster to about 1,000% faster, about 15% faster to about 500% faster, about 15% faster to about 480% faster, about 15% faster to about 460% faster, about 15% faster to about 440% faster, about 15% faster to about 420% faster, about 15% faster to about 400% faster, about 15% faster to about 380% faster, about 15% faster to about 360% faster, about 15% faster to about 340% faster, about 15% faster to about 320% faster, about 15% faster to about 300% faster, about 15% faster to about 280% faster, about 15% faster to about 260% faster, about 15% faster to about 240% faster, about 15% faster to about 220% faster, about 15% faster to about 200% faster, about 15% faster to about 180% faster, about 15% faster to about 160% faster, about 15% faster to about 140% faster, about 15% faster to about 120% faster, about 15% faster to about 100% faster, about 15% faster to about 95% faster, about 15% faster to about 90% faster, about 15% faster to about 85% faster, about 15% faster to about 80% faster, about 15% faster to about 75% faster, about 15% faster to about 70% faster, about 15% faster to about 65% faster, about 15% faster to about 60% faster, about 15% faster to about 55% faster, about 15% faster to about 50% faster, about 15% faster to about 45% faster, about 15% faster to about 40% faster, about 15% faster to about 35% faster, about 15% faster to about 30% faster, about 15% faster to about 25% faster, about 15% faster to about 20% faster, about 20% faster to about 10,000% faster, about 20% faster to about 9,000% faster, about 20% faster to about 8,000% faster, about 20% faster to about 7,000% faster, about 20% faster to about 6,000% faster, about 20% faster to about 5,000% faster, about 20% faster to about 4,000% faster, about 20% faster to about 3,000% faster, about 20% faster to about 2,000% faster, about 20% faster to about 1,000% faster, about 20% faster to about 500% faster, about 20% faster to about 480% faster, about 20% faster to about 460% faster, about 20% faster to about 440% faster, about 20% faster to about 420% faster, about 20% faster to about 400% faster, about 20% faster to about 380% faster, about 20% faster to about 360% faster, about 20% faster to about 340% faster, about 20% faster to about 320% faster, about 20% faster to about 300% faster, about 20% faster to about 280% faster, about 20% faster to about 260% faster, about 20% faster to about 240% faster, about 20% faster to about 220% faster, about 20% faster to about 200% faster, about 20% faster to about 180% faster, about 20% faster to about 160% faster, about 20% faster to about 140% faster, about 20% faster to about 120% faster, about 20% faster to about 100% faster, about 20% faster to about 95% faster, about 20% faster to about 90% faster, about 20% faster to about 85% faster, about 20% faster to about 80% faster, about 20% faster to about 75% faster, about 20% faster to about 70% faster, about 20% faster to about 65% faster, about 20% faster to about 60% faster, about 20% faster to about 55% faster, about 20% faster to about 50% faster, about 20% faster to about 45% faster, about 20% faster to about 40% faster, about 20% faster to about 35% faster, about 20% faster to about 30% faster, about 20% faster to about 25% faster, about 25% faster to about 10,000% faster, about 25% faster to about 9,000% faster, about 25% faster to about 8,000% faster, about 25% faster to about 7,000% faster, about 25% faster to about 6,000% faster, about 25% faster to about 5,000% faster, about 25% faster to about 4,000% faster, about 25% faster to about 3,000% faster, about 25% faster to about 2,000% faster, about 25% faster to about 1,000% faster, about 25% faster to about 500% faster, about 25% faster to about 480% faster, about 25% faster to about 460% faster, about 25% faster to about 440% faster, about 25% faster to about 420% faster, about 25% faster to about 400% faster, about 25% faster to about 380% faster, about 25% faster to about 360% faster, about 25% faster to about 340% faster, about 25% faster to about 320% faster, about 25% faster to about 300% faster, about 25% faster to about 280% faster, about 25% faster to about 260% faster, about 25% faster to about 240% faster, about 25% faster to about 220% faster, about 25% faster to about 200% faster, about 25% faster to about 180% faster, about 25% faster to about 160% faster, about 25% faster to about 140% faster, about 25% faster to about 120% faster, about 25% faster to about 100% faster, about 25% faster to about 95% faster, about 25% faster to about 90% faster, about 25% faster to about 85% faster, about 25% faster to about 80% faster, about 25% faster to about 75% faster, about 25% faster to about 70% faster, about 25% faster to about 65% faster, about 25% faster to about 60% faster, about 25% faster to about 55% faster, about 25% faster to about 50% faster, about 25% faster to about 45% faster, about 25% faster to about 40% faster, about 25% faster to about 35% faster, about 25% faster to about 30% faster, about 30% faster to about 10,000% faster, about 30% faster to about 9,000% faster, about 30% faster to about 8,000% faster, about 30% faster to about 7,000% faster, about 30% faster to about 6,000% faster, about 30% faster to about 5,000% faster, about 30% faster to about 4,000% faster, about 30% faster to about 3,000% faster, about 30% faster to about 2,000% faster, about 30% faster to about 1,000% faster, about 30% faster to about 500% faster, about 30% faster to about 480% faster, about 30% faster to about 460% faster, about 30% faster to about 440% faster, about 30% faster to about 420% faster, about 30% faster to about 400% faster, about 30% faster to about 380% faster, about 30% faster to about 360% faster, about 30% faster to about 340% faster, about 30% faster to about 320% faster, about 30% faster to about 300% faster, about 30% faster to about 280% faster, about 30% faster to about 260% faster, about 30% faster to about 240% faster, about 30% faster to about 220% faster, about 30% faster to about 200% faster, about 30% faster to about 180% faster, about 30% faster to about 160% faster, about 30% faster to about 140% faster, about 30% faster to about 120% faster, about 30% faster to about 100% faster, about 30% faster to about 95% faster, about 30% faster to about 90% faster, about 30% faster to about 85% faster, about 30% faster to about 80% faster, about 30% faster to about 75% faster, about 30% faster to about 70% faster, about 30% faster to about 65% faster, about 30% faster to about 60% faster, about 30% faster to about 55% faster, about 30% faster to about 50% faster, about 30% faster to about 45% faster, about 30% faster to about 40% faster, about 30% faster to about 35% faster, about 35% faster to about 10,000% faster, about 35% faster to about 9,000% faster, about 35% faster to about 8,000% faster, about 35% faster to about 7,000% faster, about 35% faster to about 6,000% faster, about 35% faster to about 5,000% faster, about 35% faster to about 4,000% faster, about 35% faster to about 3,000% faster, about 35% faster to about 2,000% faster, about 35% faster to about 1,000% faster, about 35% faster to about 500% faster, about 35% faster to about 480% faster, about 35% faster to about 460% faster, about 35% faster to about 440% faster, about 35% faster to about 420% faster, about 35% faster to about 400% faster, about 35% faster to about 380% faster, about 35% faster to about 360% faster, about 35% faster to about 340% faster, about 35% faster to about 320% faster, about 35% faster to about 300% faster, about 35% faster to about 280% faster, about 35% faster to about 260% faster, about 35% faster to about 240% faster, about 35% faster to about 220% faster, about 35% faster to about 200% faster, about 35% faster to about 180% faster, about 35% faster to about 160% faster, about 35% faster to about 140% faster, about 35% faster to about 120% faster, about 35% faster to about 100% faster, about 35% faster to about 95% faster, about 35% faster to about 90% faster, about 35% faster to about 85% faster, about 35% faster to about 80% faster, about 35% faster to about 75% faster, about 35% faster to about 70% faster, about 35% faster to about 65% faster, about 35% faster to about 60% faster, about 35% faster to about 55% faster, about 35% faster to about 50% faster, about 35% faster to about 45% faster, about 35% faster to about 40% faster, about 40% faster to about 10,000% faster, about 40% faster to about 9,000% faster, about 40% faster to about 8,000% faster, about 40% faster to about 7,000% faster, about 40% faster to about 6,000% faster, about 40% faster to about 5,000% faster, about 40% faster to about 4,000% faster, about 40% faster to about 3,000% faster, about 40% faster to about 2,000% faster, about 40% faster to about 1,000% faster, about 40% faster to about 500% faster, about 40% faster to about 480% faster, about 40% faster to about 460% faster, about 40% faster to about 440% faster, about 40% faster to about 420% faster, about 40% faster to about 400% faster, about 40% faster to about 380% faster, about 40% faster to about 360% faster, about 40% faster to about 340% faster, about 40% faster to about 320% faster, about 40% faster to about 300% faster, about 40% faster to about 280% faster, about 40% faster to about 260% faster, about 40% faster to about 240% faster, about 40% faster to about 220% faster, about 40% faster to about 200% faster, about 40% faster to about 180% faster, about 40% faster to about 160% faster, about 40% faster to about 140% faster, about 40% faster to about 120% faster, about 40% faster to about 100% faster, about 40% faster to about 95% faster, about 40% faster to about 90% faster, about 40% faster to about 85% faster, about 40% faster to about 80% faster, about 40% faster to about 75% faster, about 40% faster to about 70% faster, about 40% faster to about 65% faster, about 40% faster to about 60% faster, about 40% faster to about 55% faster, about 40% faster to about 50% faster, about 40% faster to about 45% faster, about 45% faster to about 10,000% faster, about 45% faster to about 9,000% faster, about 45% faster to about 8,000% faster, about 45% faster to about 7,000% faster, about 45% faster to about 6,000% faster, about 45% faster to about 5,000% faster, about 45% faster to about 4,000% faster, about 45% faster to about 3,000% faster, about 45% faster to about 2,000% faster, about 45% faster to about 1,000% faster, about 45% faster to about 500% faster, about 45% faster to about 480% faster, about 45% faster to about 460% faster, about 45% faster to about 440% faster, about 45% faster to about 420% faster, about 45% faster to about 400% faster, about 45% faster to about 380% faster, about 45% faster to about 360% faster, about 45% faster to about 340% faster, about 45% faster to about 320% faster, about 45% faster to about 300% faster, about 45% faster to about 280% faster, about 45% faster to about 260% faster, about 45% faster to about 240% faster, about 45% faster to about 220% faster, about 45% faster to about 200% faster, about 45% faster to about 180% faster, about 45% faster to about 160% faster, about 45% faster to about 140% faster, about 45% faster to about 120% faster, about 45% faster to about 100% faster, about 45% faster to about 95% faster, about 45% faster to about 90% faster, about 45% faster to about 85% faster, about 45% faster to about 80% faster, about 45% faster to about 75% faster, about 45% faster to about 70% faster, about 45% faster to about 65% faster, about 45% faster to about 60% faster, about 45% faster to about 55% faster, about 45% faster to about 50% faster, about 50% faster to about 10,000% faster, about 50% faster to about 9,000% faster, about 50% faster to about 8,000% faster, about 50% faster to about 7,000% faster, about 50% faster to about 6,000% faster, about 50% faster to about 5,000% faster, about 50% faster to about 4,000% faster, about 50% faster to about 3,000% faster, about 50% faster to about 2,000% faster, about 50% faster to about 1,000% faster, about 50% faster to about 500% faster, about 50% faster to about 480% faster, about 50% faster to about 460% faster, about 50% faster to about 440% faster, about 50% faster to about 420% faster, about 50% faster to about 400% faster, about 50% faster to about 380% faster, about 50% faster to about 360% faster, about 50% faster to about 340% faster, about 50% faster to about 320% faster, about 50% faster to about 300% faster, about 50% faster to about 280% faster, about 50% faster to about 260% faster, about 50% faster to about 240% faster, about 50% faster to about 220% faster, about 50% faster to about 200% faster, about 50% faster to about 180% faster, about 50% faster to about 160% faster, about 50% faster to about 140% faster, about 50% faster to about 120% faster, about 50% faster to about 100% faster, about 50% faster to about 95% faster, about 50% faster to about 90% faster, about 50% faster to about 85% faster, about 50% faster to about 80% faster, about 50% faster to about 75% faster, about 50% faster to about 70% faster, about 50% faster to about 65% faster, about 50% faster to about 60% faster, about 50% faster to about 55% faster, about 55% faster to about 10,000% faster, about 55% faster to about 9,000% faster, about 55% faster to about 8,000% faster, about 55% faster to about 7,000% faster, about 55% faster to about 6,000% faster, about 55% faster to about 5,000% faster, about 55% faster to about 4,000% faster, about 55% faster to about 3,000% faster, about 55% faster to about 2,000% faster, about 55% faster to about 1,000% faster, about 55% faster to about 500% faster, about 55% faster to about 480% faster, about 55% faster to about 460% faster, about 55% faster to about 440% faster, about 55% faster to about 420% faster, about 55% faster to about 400% faster, about 55% faster to about 380% faster, about 55% faster to about 360% faster, about 55% faster to about 340% faster, about 55% faster to about 320% faster, about 55% faster to about 300% faster, about 55% faster to about 280% faster, about 55% faster to about 260% faster, about 55% faster to about 240% faster, about 55% faster to about 220% faster, about 55% faster to about 200% faster, about 55% faster to about 180% faster, about 55% faster to about 160% faster, about 55% faster to about 140% faster, about 55% faster to about 120% faster, about 55% faster to about 100% faster, about 55% faster to about 95% faster, about 55% faster to about 90% faster, about 55% faster to about 85% faster, about 55% faster to about 80% faster, about 55% faster to about 75% faster, about 55% faster to about 70% faster, about 55% faster to about 65% faster, about 55% faster to about 60% faster, about 60% faster to about 10,000% faster, about 60% faster to about 9,000% faster, about 60% faster to about 8,000% faster, about 60% faster to about 7,000% faster, about 60% faster to about 6,000% faster, about 60% faster to about 5,000% faster, about 60% faster to about 4,000% faster, about 60% faster to about 3,000% faster, about 60% faster to about 2,000% faster, about 60% faster to about 1,000% faster, about 60% faster to about 500% faster, about 60% faster to about 480% faster, about 60% faster to about 460% faster, about 60% faster to about 440% faster, about 60% faster to about 420% faster, about 60% faster to about 400% faster, about 60% faster to about 380% faster, about 60% faster to about 360% faster, about 60% faster to about 340% faster, about 60% faster to about 320% faster, about 60% faster to about 300% faster, about 60% faster to about 280% faster, about 60% faster to about 260% faster, about 60% faster to about 240% faster, about 60% faster to about 220% faster, about 60% faster to about 200% faster, about 60% faster to about 180% faster, about 60% faster to about 160% faster, about 60% faster to about 140% faster, about 60% faster to about 120% faster, about 60% faster to about 100% faster, about 60% faster to about 95% faster, about 60% faster to about 90% faster, about 60% faster to about 85% faster, about 60% faster to about 80% faster, about 60% faster to about 75% faster, about 60% faster to about 70% faster, about 60% faster to about 65% faster, about 65% faster to about 10,000% faster, about 65% faster to about 9,000% faster, about 65% faster to about 8,000% faster, about 65% faster to about 7,000% faster, about 65% faster to about 6,000% faster, about 65% faster to about 5,000% faster, about 65% faster to about 4,000% faster, about 65% faster to about 3,000% faster, about 65% faster to about 2,000% faster, about 65% faster to about 1,000% faster, about 65% faster to about 500% faster, about 65% faster to about 480% faster, about 65% faster to about 460% faster, about 65% faster to about 440% faster, about 65% faster to about 420% faster, about 65% faster to about 400% faster, about 65% faster to about 380% faster, about 65% faster to about 360% faster, about 65% faster to about 340% faster, about 65% faster to about 320% faster, about 65% faster to about 300% faster, about 65% faster to about 280% faster, about 65% faster to about 260% faster, about 65% faster to about 240% faster, about 65% faster to about 220% faster, about 65% faster to about 200% faster, about 65% faster to about 180% faster, about 65% faster to about 160% faster, about 65% faster to about 140% faster, about 65% faster to about 120% faster, about 65% faster to about 100% faster, about 65% faster to about 95% faster, about 65% faster to about 90% faster, about 65% faster to about 85% faster, about 65% faster to about 80% faster, about 65% faster to about 75% faster, about 65% faster to about 70% faster, about 70% faster to about 10,000% faster, about 70% faster to about 9,000% faster, about 70% faster to about 8,000% faster, about 70% faster to about 7,000% faster, about 70% faster to about 6,000% faster, about 70% faster to about 5,000% faster, about 70% faster to about 4,000% faster, about 70% faster to about 3,000% faster, about 70% faster to about 2,000% faster, about 70% faster to about 1,000% faster, about 70% faster to about 500% faster, about 70% faster to about 480% faster, about 70% faster to about 460% faster, about 70% faster to about 440% faster, about 70% faster to about 420% faster, about 70% faster to about 400% faster, about 70% faster to about 380% faster, about 70% faster to about 360% faster, about 70% faster to about 340% faster, about 70% faster to about 320% faster, about 70% faster to about 300% faster, about 70% faster to about 280% faster, about 70% faster to about 260% faster, about 70% faster to about 240% faster, about 70% faster to about 220% faster, about 70% faster to about 200% faster, about 70% faster to about 180% faster, about 70% faster to about 160% faster, about 70% faster to about 140% faster, about 70% faster to about 120% faster, about 70% faster to about 100% faster, about 70% faster to about 95% faster, about 70% faster to about 90% faster, about 70% faster to about 85% faster, about 70% faster to about 80% faster, about 70% faster to about 75% faster, about 75% faster to about 10,000% faster, about 75% faster to about 9,000% faster, about 75% faster to about 8,000% faster, about 75% faster to about 7,000% faster, about 75% faster to about 6,000% faster, about 75% faster to about 5,000% faster, about 75% faster to about 4,000% faster, about 75% faster to about 3,000% faster, about 75% faster to about 2,000% faster, about 75% faster to about 1,000% faster, about 75% faster to about 500% faster, about 75% faster to about 480% faster, about 75% faster to about 460% faster, about 75% faster to about 440% faster, about 75% faster to about 420% faster, about 75% faster to about 400% faster, about 75% faster to about 380% faster, about 75% faster to about 360% faster, about 75% faster to about 340% faster, about 75% faster to about 320% faster, about 75% faster to about 300% faster, about 75% faster to about 280% faster, about 75% faster to about 260% faster, about 75% faster to about 240% faster, about 75% faster to about 220% faster, about 75% faster to about 200% faster, about 75% faster to about 180% faster, about 75% faster to about 160% faster, about 75% faster to about 140% faster, about 75% faster to about 120% faster, about 75% faster to about 100% faster, about 75% faster to about 95% faster, about 75% faster to about 90% faster, about 75% faster to about 85% faster, about 75% faster to about 80% faster, about 80% faster to about 10,000% faster, about 80% faster to about 9,000% faster, about 80% faster to about 8,000% faster, about 80% faster to about 7,000% faster, about 80% faster to about 6,000% faster, about 80% faster to about 5,000% faster, about 80% faster to about 4,000% faster, about 80% faster to about 3,000% faster, about 80% faster to about 2,000% faster, about 80% faster to about 1,000% faster, about 80% faster to about 500% faster, about 80% faster to about 480% faster, about 80% faster to about 460% faster, about 80% faster to about 440% faster, about 80% faster to about 420% faster, about 80% faster to about 400% faster, about 80% faster to about 380% faster, about 80% faster to about 360% faster, about 80% faster to about 340% faster, about 80% faster to about 320% faster, about 80% faster to about 300% faster, about 80% faster to about 280% faster, about 80% faster to about 260% faster, about 80% faster to about 240% faster, about 80% faster to about 220% faster, about 80% faster to about 200% faster, about 80% faster to about 180% faster, about 80% faster to about 160% faster, about 80% faster to about 140% faster, about 80% faster to about 120% faster, about 80% faster to about 100% faster, about 80% faster to about 95% faster, about 80% faster to about 90% faster, about 80% faster to about 85% faster, about 85% faster to about 10,000% faster, about 85% faster to about 9,000% faster, about 85% faster to about 8,000% faster, about 85% faster to about 7,000% faster, about 85% faster to about 6,000% faster, about 85% faster to about 5,000% faster, about 85% faster to about 4,000% faster, about 85% faster to about 3,000% faster, about 85% faster to about 2,000% faster, about 85% faster to about 1,000% faster, about 85% faster to about 500% faster, about 85% faster to about 480% faster, about 85% faster to about 460% faster, about 85% faster to about 440% faster, about 85% faster to about 420% faster, about 85% faster to about 400% faster, about 85% faster to about 380% faster, about 85% faster to about 360% faster, about 85% faster to about 340% faster, about 85% faster to about 320% faster, about 85% faster to about 300% faster, about 85% faster to about 280% faster, about 85% faster to about 260% faster, about 85% faster to about 240% faster, about 85% faster to about 220% faster, about 85% faster to about 200% faster, about 85% faster to about 180% faster, about 85% faster to about 160% faster, about 85% faster to about 140% faster, about 85% faster to about 120% faster, about 85% faster to about 100% faster, about 85% faster to about 95% faster, about 85% faster to about 90% faster, about 90% faster to about 10,000% faster, about 90% faster to about 9,000% faster, about 90% faster to about 8,000% faster, about 90% faster to about 7,000% faster, about 90% faster to about 6,000% faster, about 90% faster to about 5,000% faster, about 90% faster to about 4,000% faster, about 90% faster to about 3,000% faster, about 90% faster to about 2,000% faster, about 90% faster to about 1,000% faster, about 90% faster to about 500% faster, about 90% faster to about 480% faster, about 90% faster to about 460% faster, about 90% faster to about 440% faster, about 90% faster to about 420% faster, about 90% faster to about 400% faster, about 90% faster to about 380% faster, about 90% faster to about 360% faster, about 90% faster to about 340% faster, about 90% faster to about 320% faster, about 90% faster to about 300% faster, about 90% faster to about 280% faster, about 90% faster to about 260% faster, about 90% faster to about 240% faster, about 90% faster to about 220% faster, about 90% faster to about 200% faster, about 90% faster to about 180% faster, about 90% faster to about 160% faster, about 90% faster to about 140% faster, about 90% faster to about 120% faster, about 90% faster to about 100% faster, about 90% faster to about 95% faster, about 95% faster to about 10,000% faster, about 95% faster to about 9,000% faster, about 95% faster to about 8,000% faster, about 95% faster to about 7,000% faster, about 95% faster to about 6,000% faster, about 95% faster to about 5,000% faster, about 95% faster to about 4,000% faster, about 95% faster to about 3,000% faster, about 95% faster to about 2,000% faster, about 95% faster to about 1,000% faster, about 95% faster to about 500% faster, about 95% faster to about 480% faster, about 95% faster to about 460% faster, about 95% faster to about 440% faster, about 95% faster to about 420% faster, about 95% faster to about 400% faster, about 95% faster to about 380% faster, about 95% faster to about 360% faster, about 95% faster to about 340% faster, about 95% faster to about 320% faster, about 95% faster to about 300% faster, about 95% faster to about 280% faster, about 95% faster to about 260% faster, about 95% faster to about 240% faster, about 95% faster to about 220% faster, about 95% faster to about 200% faster, about 95% faster to about 180% faster, about 95% faster to about 160% faster, about 95% faster to about 140% faster, about 95% faster to about 120% faster, about 95% faster to about 100% faster, about 100% faster to about 10,000% faster, about 100% faster to about 9,000% faster, about 100% faster to about 8,000% faster, about 100% faster to about 7,000% faster, about 100% faster to about 6,000% faster, about 100% faster to about 5,000% faster, about 100% faster to about 4,000% faster, about 100% faster to about 3,000% faster, about 100% faster to about 2,000% faster, about 100% faster to about 1,000% faster, about 100% faster to about 500% faster, about 100% faster to about 480% faster, about 100% faster to about 460% faster, about 100% faster to about 440% faster, about 100% faster to about 420% faster, about 100% faster to about 400% faster, about 100% faster to about 380% faster, about 100% faster to about 360% faster, about 100% faster to about 340% faster, about 100% faster to about 320% faster, about 100% faster to about 300% faster, about 100% faster to about 280% faster, about 100% faster to about 260% faster, about 100% faster to about 240% faster, about 100% faster to about 220% faster, about 100% faster to about 200% faster, about 100% faster to about 180% faster, about 100% faster to about 160% faster, about 100% faster to about 140% faster, about 100% faster to about 120% faster, about 120% faster to about 10,000% faster, about 120% faster to about 9,000% faster, about 120% faster to about 8,000% faster, about 120% faster to about 7,000% faster, about 120% faster to about 6,000% faster, about 120% faster to about 5,000% faster, about 120% faster to about 4,000% faster, about 120% faster to about 3,000% faster, about 120% faster to about 2,000% faster, about 120% faster to about 1,000% faster, about 120% faster to about 500% faster, about 120% faster to about 480% faster, about 120% faster to about 460% faster, about 120% faster to about 440% faster, about 120% faster to about 420% faster, about 120% faster to about 400% faster, about 120% faster to about 380% faster, about 120% faster to about 360% faster, about 120% faster to about 340% faster, about 120% faster to about 320% faster, about 120% faster to about 300% faster, about 120% faster to about 280% faster, about 120% faster to about 260% faster, about 120% faster to about 240% faster, about 120% faster to about 220% faster, about 120% faster to about 200% faster, about 120% faster to about 180% faster, about 120% faster to about 160% faster, about 120% faster to about 140% faster, about 140% faster to about 10,000% faster, about 140% faster to about 9,000% faster, about 140% faster to about 8,000% faster, about 140% faster to about 7,000% faster, about 140% faster to about 6,000% faster, about 140% faster to about 5,000% faster, about 140% faster to about 4,000% faster, about 140% faster to about 3,000% faster, about 140% faster to about 2,000% faster, about 140% faster to about 1,000% faster, about 140% faster to about 500% faster, about 140% faster to about 480% faster, about 140% faster to about 460% faster, about 140% faster to about 440% faster, about 140% faster to about 420% faster, about 140% faster to about 400% faster, about 140% faster to about 380% faster, about 140% faster to about 360% faster, about 140% faster to about 340% faster, about 140% faster to about 320% faster, about 140% faster to about 300% faster, about 140% faster to about 280% faster, about 140% faster to about 260% faster, about 140% faster to about 240% faster, about 140% faster to about 220% faster, about 140% faster to about 200% faster, about 140% faster to about 180% faster, about 140% faster to about 160% faster, about 160% faster to about 10,000% faster, about 160% faster to about 9,000% faster, about 160% faster to about 8,000% faster, about 160% faster to about 7,000% faster, about 160% faster to about 6,000% faster, about 160% faster to about 5,000% faster, about 160% faster to about 4,000% faster, about 160% faster to about 3,000% faster, about 160% faster to about 2,000% faster, about 160% faster to about 1,000% faster, about 160% faster to about 500% faster, about 160% faster to about 480% faster, about 160% faster to about 460% faster, about 160% faster to about 440% faster, about 160% faster to about 420% faster, about 160% faster to about 400% faster, about 160% faster to about 380% faster, about 160% faster to about 360% faster, about 160% faster to about 340% faster, about 160% faster to about 320% faster, about 160% faster to about 300% faster, about 160% faster to about 280% faster, about 160% faster to about 260% faster, about 160% faster to about 240% faster, about 160% faster to about 220% faster, about 160% faster to about 200% faster, about 160% faster to about 180% faster, about 180% faster to about 10,000% faster, about 180% faster to about 9,000% faster, about 180% faster to about 8,000% faster, about 180% faster to about 7,000% faster, about 180% faster to about 6,000% faster, about 180% faster to about 5,000% faster, about 180% faster to about 4,000% faster, about 180% faster to about 3,000% faster, about 180% faster to about 2,000% faster, about 180% faster to about 1,000% faster, about 180% faster to about 500% faster, about 180% faster to about 480% faster, about 180% faster to about 460% faster, about 180% faster to about 440% faster, about 180% faster to about 420% faster, about 180% faster to about 400% faster, about 180% faster to about 380% faster, about 180% faster to about 360% faster, about 180% faster to about 340% faster, about 180% faster to about 320% faster, about 180% faster to about 300% faster, about 180% faster to about 280% faster, about 180% faster to about 260% faster, about 180% faster to about 240% faster, about 180% faster to about 220% faster, about 180% faster to about 200% faster, about 200% faster to about 10,000% faster, about 200% faster to about 9,000% faster, about 200% faster to about 8,000% faster, about 200% faster to about 7,000% faster, about 200% faster to about 6,000% faster, about 200% faster to about 5,000% faster, about 200% faster to about 4,000% faster, about 200% faster to about 3,000% faster, about 200% faster to about 2,000% faster, about 200% faster to about 1,000% faster, about 200% faster to about 500% faster, about 200% faster to about 480% faster, about 200% faster to about 460% faster, about 200% faster to about 440% faster, about 200% faster to about 420% faster, about 200% faster to about 400% faster, about 200% faster to about 380% faster, about 200% faster to about 360% faster, about 200% faster to about 340% faster, about 200% faster to about 320% faster, about 200% faster to about 300% faster, about 200% faster to about 280% faster, about 200% faster to about 260% faster, about 200% faster to about 240% faster, about 200% faster to about 220% faster, about 220% faster to about 10,000% faster, about 220% faster to about 9,000% faster, about 220% faster to about 8,000% faster, about 220% faster to about 7,000% faster, about 220% faster to about 6,000% faster, about 220% faster to about 5,000% faster, about 220% faster to about 4,000% faster, about 220% faster to about 3,000% faster, about 220% faster to about 2,000% faster, about 220% faster to about 1,000% faster, about 220% faster to about 500% faster, about 220% faster to about 480% faster, about 220% faster to about 460% faster, about 220% faster to about 440% faster, about 220% faster to about 420% faster, about 220% faster to about 400% faster, about 220% faster to about 380% faster, about 220% faster to about 360% faster, about 220% faster to about 340% faster, about 220% faster to about 320% faster, about 220% faster to about 300% faster, about 220% faster to about 280% faster, about 220% faster to about 260% faster, about 220% faster to about 240% faster, about 240% faster to about 10,000% faster, about 240% faster to about 9,000% faster, about 240% faster to about 8,000% faster, about 240% faster to about 7,000% faster, about 240% faster to about 6,000% faster, about 240% faster to about 5,000% faster, about 240% faster to about 4,000% faster, about 240% faster to about 3,000% faster, about 240% faster to about 2,000% faster, about 240% faster to about 1,000% faster, about 240% faster to about 500% faster, about 240% faster to about 480% faster, about 240% faster to about 460% faster, about 240% faster to about 440% faster, about 240% faster to about 420% faster, about 240% faster to about 400% faster, about 240% faster to about 380% faster, about 240% faster to about 360% faster, about 240% faster to about 340% faster, about 240% faster to about 320% faster, about 240% faster to about 300% faster, about 240% faster to about 280% faster, about 240% faster to about 260% faster, about 260% faster to about 10,000% faster, about 260% faster to about 9,000% faster, about 260% faster to about 8,000% faster, about 260% faster to about 7,000% faster, about 260% faster to about 6,000% faster, about 260% faster to about 5,000% faster, about 260% faster to about 4,000% faster, about 260% faster to about 3,000% faster, about 260% faster to about 2,000% faster, about 260% faster to about 1,000% faster, about 260% faster to about 500% faster, about 260% faster to about 480% faster, about 260% faster to about 460% faster, about 260% faster to about 440% faster, about 260% faster to about 420% faster, about 260% faster to about 400% faster, about 260% faster to about 380% faster, about 260% faster to about 360% faster, about 260% faster to about 340% faster, about 260% faster to about 320% faster, about 260% faster to about 300% faster, about 260% faster to about 280% faster, about 280% faster to about 10,000% faster, about 280% faster to about 9,000% faster, about 280% faster to about 8,000% faster, about 280% faster to about 7,000% faster, about 280% faster to about 6,000% faster, about 280% faster to about 5,000% faster, about 280% faster to about 4,000% faster, about 280% faster to about 3,000% faster, about 280% faster to about 2,000% faster, about 280% faster to about 1,000% faster, about 280% faster to about 500% faster, about 280% faster to about 480% faster, about 280% faster to about 460% faster, about 280% faster to about 440% faster, about 280% faster to about 420% faster, about 280% faster to about 400% faster, about 280% faster to about 380% faster, about 280% faster to about 360% faster, about 280% faster to about 340% faster, about 280% faster to about 320% faster, about 280% faster to about 300% faster, about 300% faster to about 10,000% faster, about 300% faster to about 9,000% faster, about 300% faster to about 8,000% faster, about 300% faster to about 7,000% faster, about 300% faster to about 6,000% faster, about 300% faster to about 5,000% faster, about 300% faster to about 4,000% faster, about 300% faster to about 3,000% faster, about 300% faster to about 2,000% faster, about 300% faster to about 1,000% faster, about 300% faster to about 500% faster, about 300% faster to about 480% faster, about 300% faster to about 460% faster, about 300% faster to about 440% faster, about 300% faster to about 420% faster, about 300% faster to about 400% faster, about 300% faster to about 380% faster, about 300% faster to about 360% faster, about 300% faster to about 340% faster, about 300% faster to about 320% faster, about 320% faster to about 10,000% faster, about 320% faster to about 9,000% faster, about 320% faster to about 8,000% faster, about 320% faster to about 7,000% faster, about 320% faster to about 6,000% faster, about 320% faster to about 5,000% faster, about 320% faster to about 4,000% faster, about 320% faster to about 3,000% faster, about 320% faster to about 2,000% faster, about 320% faster to about 1,000% faster, about 320% faster to about 500% faster, about 320% faster to about 480% faster, about 320% faster to about 460% faster, about 320% faster to about 440% faster, about 320% faster to about 420% faster, about 320% faster to about 400% faster, about 320% faster to about 380% faster, about 320% faster to about 360% faster, about 320% faster to about 340% faster, about 340% faster to about 10,000% faster, about 340% faster to about 9,000% faster, about 340% faster to about 8,000% faster, about 340% faster to about 7,000% faster, about 340% faster to about 6,000% faster, about 340% faster to about 5,000% faster, about 340% faster to about 4,000% faster, about 340% faster to about 3,000% faster, about 340% faster to about 2,000% faster, about 340% faster to about 1,000% faster, about 340% faster to about 500% faster, about 340% faster to about 480% faster, about 340% faster to about 460% faster, about 340% faster to about 440% faster, about 340% faster to about 420% faster, about 340% faster to about 400% faster, about 340% faster to about 380% faster, about 340% faster to about 360% faster, about 360% faster to about 10,000% faster, about 360% faster to about 9,000% faster, about 360% faster to about 8,000% faster, about 360% faster to about 7,000% faster, about 360% faster to about 6,000% faster, about 360% faster to about 5,000% faster, about 360% faster to about 4,000% faster, about 360% faster to about 3,000% faster, about 360% faster to about 2,000% faster, about 360% faster to about 1,000% faster, about 360% faster to about 500% faster, about 360% faster to about 480% faster, about 360% faster to about 460% faster, about 360% faster to about 440% faster, about 360% faster to about 420% faster, about 360% faster to about 400% faster, about 360% faster to about 380% faster, about 380% faster to about 10,000% faster, about 380% faster to about 9,000% faster, about 380% faster to about 8,000% faster, about 380% faster to about 7,000% faster, about 380% faster to about 6,000% faster, about 380% faster to about 5,000% faster, about 380% faster to about 4,000% faster, about 380% faster to about 3,000% faster, about 380% faster to about 2,000% faster, about 380% faster to about 1,000% faster, about 380% faster to about 500% faster, about 380% faster to about 480% faster, about 380% faster to about 460% faster, about 380% faster to about 440% faster, about 380% faster to about 420% faster, about 380% faster to about 400% faster, about 400% faster to about 10,000% faster, about 400% faster to about 9,000% faster, about 400% faster to about 8,000% faster, about 400% faster to about 7,000% faster, about 400% faster to about 6,000% faster, about 400% faster to about 5,000% faster, about 400% faster to about 4,000% faster, about 400% faster to about 3,000% faster, about 400% faster to about 2,000% faster, about 400% faster to about 1,000% faster, about 400% faster to about 500% faster, about 400% faster to about 480% faster, about 400% faster to about 460% faster, about 400% faster to about 440% faster, about 400% faster to about 420% faster, about 420% faster to about 10,000% faster, about 420% faster to about 9,000% faster, about 420% faster to about 8,000% faster, about 420% faster to about 7,000% faster, about 420% faster to about 6,000% faster, about 420% faster to about 5,000% faster, about 420% faster to about 4,000% faster, about 420% faster to about 3,000% faster, about 420% faster to about 2,000% faster, about 420% faster to about 1,000% faster, about 420% faster to about 500% faster, about 420% faster to about 480% faster, about 420% faster to about 460% faster, about 420% faster to about 440% faster, about 440% faster to about 10,000% faster, about 440% faster to about 9,000% faster, about 440% faster to about 8,000% faster, about 440% faster to about 7,000% faster, about 440% faster to about 6,000% faster, about 440% faster to about 5,000% faster, about 440% faster to about 4,000% faster, about 440% faster to about 3,000% faster, about 440% faster to about 2,000% faster, about 440% faster to about 1,000% faster, about 440% faster to about 500% faster, about 440% faster to about 480% faster, about 440% faster to about 460% faster, about 460% faster to about 10,000% faster, about 460% faster to about 9,000% faster, about 460% faster to about 8,000% faster, about 460% faster to about 7,000% faster, about 460% faster to about 6,000% faster, about 460% faster to about 5,000% faster, about 460% faster to about 4,000% faster, about 460% faster to about 3,000% faster, about 460% faster to about 2,000% faster, about 460% faster to about 1,000% faster, about 460% faster to about 500% faster, about 460% faster to about 480% faster, about 480% faster to about 10,000% faster, about 480% faster to about 9,000% faster, about 480% faster to about 8,000% faster, about 480% faster to about 7,000% faster, about 480% faster to about 6,000% faster, about 480% faster to about 5,000% faster, about 480% faster to about 4,000% faster, about 480% faster to about 3,000% faster, about 480% faster to about 2,000% faster, about 480% faster to about 1,000% faster, about 480% faster to about 500% faster, about 500% faster to about 10,000% faster, about 500% faster to about 9,000% faster, about 500% faster to about 8,000% faster, about 500% faster to about 7,000% faster, about 500% faster to about 6,000% faster, about 500% faster to about 5,000% faster, about 500% faster to about 4,000% faster, about 500% faster to about 3,000% faster, about 500% faster to about 2,000% faster, about 500% faster to about 1,000% faster, about 1,000% faster to about 10,000% faster, about 1,000% faster to about 9,000% faster, about 1,000% faster to about 8,000% faster, about 1,000% faster to about 7,000% faster, about 1,000% faster to about 6,000% faster, about 1,000% faster to about 5,000% faster, about 1,000% faster to about 4,000% faster, about 1,000% faster to about 3,000% faster, about 1,000% faster to about 2,000% faster, about 2,000% faster to about 10,000% faster, about 2,000% faster to about 9,000% faster, about 2,000% faster to about 8,000% faster, about 2,000% faster to about 7,000% faster, about 2,000% faster to about 6,000% faster, about 2,000% faster to about 5,000% faster, about 2,000% faster to about 4,000% faster, about 2,000% faster to about 3,000% faster, about 3,000% faster to about 10,000% faster, about 3,000% faster to about 9,000% faster, about 3,000% faster to about 8,000% faster, about 3,000% faster to about 7,000% faster, about 3,000% faster to about 6,000% faster, about 3,000% faster to about 5,000% faster, about 3,000% faster to about 4,000% faster, about 4,000% faster to about 10,000% faster, about 4,000% faster to about 9,000% faster, about 4,000% faster to about 8,000% faster, about 4,000% faster to about 7,000% faster, about 4,000% faster to about 6,000% faster, about 4,000% faster to about 5,000% faster, about 5,000% faster to about 10,000% faster, about 5,000% faster to about 9,000% faster, about 5,000% faster to about 8,000% faster, about 5,000% faster to about 7,000% faster, about 5,000% faster to about 6,000% faster, about 6,000% faster to about 10,000% faster, about 6,000% faster to about 9,000% faster, about 6,000% faster to about 8,000% faster, about 6,000% faster to about 7,000% faster, about 7,000% faster to about 10,000% faster, about 7,000% faster to about 9,000% faster, about 7,000% faster to about 8,000% faster, about 8,000% faster to about 10,000% faster, about 8,000% faster to about 9,000% faster, or about 9,000% faster to about 10,000% faster) than the dissociation rate at a pH of about 7.0 to about 8.0 (e.g., about 7.0 to about 7.9, about 7.0 to about 7.8, about 7.0 to about 7.7, about 7.0 to about 7.6, about 7.0 to about 7.5, about 7.0 to about 7.4, about 7.0 to about 7.3, about 7.0 to about 7.2, about 7.0 to about 7.1, about 7.1 to about 8.0, about 7.1 to about 7.9, about 7.1 to about 7.8, about 7.1 to about 7.7, about 7.1 to about 7.6, about 7.1 to about 7.5, about 7.1 to about 7.4, about 7.1 to about 7.3, about 7.1 to about 7.2, about 7.2 to about 8.0, about 7.2 to about 7.9, about 7.2 to about 7.8, about 7.2 to about 7.7, about 7.2 to about 7.6, about 7.2 to about 7.5, about 7.2 to about 7.4, about 7.2 to about 7.3, about 7.3 to about 8.0, about 7.3 to about 7.9, about 7.3 to about 7.8, about 7.3 to about 7.7, about 7.3 to about 7.6, about 7.3 to about 7.5, about 7.3 to about 7.4, about 7.4 to about 8.0, about 7.4 to about 7.9, about 7.4 to about 7.8, about 7.4 to about 7.7, about 7.4 to about 7.6, about 7.4 to about 7.5, about 7.5 to about 8.0, about 7.5 to about 7.9, about 7.5 to about 7.8, about 7.5 to about 7.7, about 7.5 to about 7.6, about 7.6 to about 8.0, about 7.6 to about 7.9, about 7.6 to about 7.8, about 7.6 to about 7.7, about 7.7 to about 8.0, about 7.7 to about 7.9, about 7.7 to about 7.8, about 7.8 to about 8.0, about 7.8 to about 7.9, or about 7.9 to about 8.0).

In some embodiments of any of the antigen-binding protein constructs (ABPCs) described herein, the dissociation constant (K_{D}) of the first antigen-binding domain (and optionally the second antigen-binding domain, if present) at a pH of about 4.0 to about 6.5 (e.g., any of the subranges of this range described herein) is greater (e.g., detectably greater) (e.g., at least 5% greater, at least 10% greater, at least 15% greater, at least 20% greater, at least 25% greater, at least 30% greater, at least 35% greater, at least 40% greater, at least 45% greater, at least 50% greater, at least 55% greater, at least 60% greater, at least 65% greater, at least 70% greater, at least 80% greater, at least 85% greater, at least 90% greater, at least 95% greater, at least 100% greater, at least 120% greater, at least 140% greater, at least 160% greater, at least 180% greater, at least 200% greater, at least 220% greater, at least 240% greater, at least 260% greater, at least 280% greater, at least 300% greater, at least 320% greater, at least 340% greater, at least 360% greater, at least 380% greater, at least 400% greater, at least 420% greater, at least 440% greater, at least 460% greater, at least 480% greater, at least 500% greater, at least 1,000% greater, at least 2,000% greater, at least 3,000% greater, at least 4,000% greater, at least 5,000% greater, at least 6,000% greater, at least 7,000% greater, at least 8,000% greater, at least 9,000% greater, or at least 10,000% greater, or about 5% greater to about 10,000% greater, about 5% greater to about 9,000% greater, about 5% greater to about 8,000% greater, about 5% greater to about 7,000% greater, about 5% greater to about 6,000% greater, about 5% greater to about 5,000% greater, about 5% greater to about 4,000% greater, about 5% greater to about 3,000% greater, about 5% greater to about 2,000% greater, about 5% greater to about 1,000% greater, about 5% greater to about 500% greater, about 5% greater to about 480% greater, about 5% greater to about 460% greater, about 5% greater to about 440% greater, about 5% greater to about 420% greater, about 5% greater to about 400% greater, about 5% greater to about 380% greater, about 5% greater to about 360% greater, about 5% greater to about 340% greater, about 5% greater to about 320% greater, about 5% greater to about 300% greater, about 5% greater to about 280% greater, about 5% greater to about 260% greater, about 5% greater to about 240% greater, about 5% greater to about 220% greater, about 5% greater to about 200% greater, about 5% greater to about 180% greater, about 5% greater to about 160% greater, about 5% greater to about 140% greater, about 5% greater to about 120% greater, about 5% greater to about 100% greater, about 5% greater to about 95% greater, about 5% greater to about 90% greater, about 5% greater to about 85% greater, about 5% greater to about 80% greater, about 5% greater to about 75% greater, about 5% greater to about 70% greater, about 5% greater to about 65% greater, about 5% greater to about 60% greater, about 5% greater to about 55% greater, about 5% greater to about 50% greater, about 5% greater to about 45% greater, about 5% greater to about 40% greater, about 5% greater to about 35% greater, about 5% greater to about 30% greater, about 5% greater to about 25% greater, about 5% greater to about 20% greater, about 5% greater to about 15% greater, about 5% greater to about 10% greater, about 10% greater to about 10,000% greater, about 10% greater to about 9,000% greater, about 10% greater to about 8,000% greater, about 10% greater to about 7,000% greater, about 10% greater to about 6,000% greater, about 10% greater to about 5,000% greater, about 10% greater to about 4,000% greater, about 10% greater to about 3,000% greater, about 10% greater to about 2,000% greater, about 10% greater to about 1,000% greater, about 10% greater to about 500% greater, about 10% greater to about 480% greater, about 10% greater to about 460% greater, about 10% greater to about 440% greater, about 10% greater to about 420% greater, about 10% greater to about 400% greater, about 10% greater to about 380% greater, about 10% greater to about 360% greater, about 10% greater to about 340% greater, about 10% greater to about 320% greater, about 10% greater to about 300% greater, about 10% greater to about 280% greater, about 10% greater to about 260% greater, about 10% greater to about 240% greater, about 10% greater to about 220% greater, about 10% greater to about 200% greater, about 10% greater to about 180% greater, about 10% greater to about 160% greater, about 10% greater to about 140% greater, about 10% greater to about 120% greater, about 10% greater to about 100% greater, about 10% greater to about 95% greater, about 10% greater to about 90% greater, about 10% greater to about 85% greater, about 10% greater to about 80% greater, about 10% greater to about 75% greater, about 10% greater to about 70% greater, about 10% greater to about 65% greater, about 10% greater to about 60% greater, about 10% greater to about 55% greater, about 10% greater to about 50% greater, about 10% greater to about 45% greater, about 10% greater to about 40% greater, about 10% greater to about 35% greater, about 10% greater to about 30% greater, about 10% greater to about 25% greater, about 10% greater to about 20% greater, about 10% greater to about 15% greater, about 15% greater to about 10,000% greater, about 15% greater to about 9,000% greater, about 15% greater to about 8,000% greater, about 15% greater to about 7,000% greater, about 15% greater to about 6,000% greater, about 15% greater to about 5,000% greater, about 15% greater to about 4,000% greater, about 15% greater to about 3,000% greater, about 15% greater to about 2,000% greater, about 15% greater to about 1,000% greater, about 15% greater to about 500% greater, about 15% greater to about 480% greater, about 15% greater to about 460% greater, about 15% greater to about 440% greater, about 15% greater to about 420% greater, about 15% greater to about 400% greater, about 15% greater to about 380% greater, about 15% greater to about 360% greater, about 15% greater to about 340% greater, about 15% greater to about 320% greater, about 15% greater to about 300% greater, about 15% greater to about 280% greater, about 15% greater to about 260% greater, about 15% greater to about 240% greater, about 15% greater to about 220% greater, about 15% greater to about 200% greater, about 15% greater to about 180% greater, about 15% greater to about 160% greater, about 15% greater to about 140% greater, about 15% greater to about 120% greater, about 15% greater to about 100% greater, about 15% greater to about 95% greater, about 15% greater to about 90% greater, about 15% greater to about 85% greater, about 15% greater to about 80% greater, about 15% greater to about 75% greater, about 15% greater to about 70% greater, about 15% greater to about 65% greater, about 15% greater to about 60% greater, about 15% greater to about 55% greater, about 15% greater to about 50% greater, about 15% greater to about 45% greater, about 15% greater to about 40% greater, about 15% greater to about 35% greater, about 15% greater to about 30% greater, about 15% greater to about 25% greater, about 15% greater to about 20% greater, about 20% greater to about 10,000% greater, about 20% greater to about 9,000% greater, about 20% greater to about 8,000% greater, about 20% greater to about 7,000% greater, about 20% greater to about 6,000% greater, about 20% greater to about 5,000% greater, about 20% greater to about 4,000% greater, about 20% greater to about 3,000% greater, about 20% greater to about 2,000% greater, about 20% greater to about 1,000% greater, about 20% greater to about 500% greater, about 20% greater to about 480% greater, about 20% greater to about 460% greater, about 20% greater to about 440% greater, about 20% greater to about 420% greater, about 20% greater to about 400% greater, about 20% greater to about 380% greater, about 20% greater to about 360% greater, about 20% greater to about 340% greater, about 20% greater to about 320% greater, about 20% greater to about 300% greater, about 20% greater to about 280% greater, about 20% greater to about 260% greater, about 20% greater to about 240% greater, about 20% greater to about 220% greater, about 20% greater to about 200% greater, about 20% greater to about 180% greater, about 20% greater to about 160% greater, about 20% greater to about 140% greater, about 20% greater to about 120% greater, about 20% greater to about 100% greater, about 20% greater to about 95% greater, about 20% greater to about 90% greater, about 20% greater to about 85% greater, about 20% greater to about 80% greater, about 20% greater to about 75% greater, about 20% greater to about 70% greater, about 20% greater to about 65% greater, about 20% greater to about 60% greater, about 20% greater to about 55% greater, about 20% greater to about 50% greater, about 20% greater to about 45% greater, about 20% greater to about 40% greater, about 20% greater to about 35% greater, about 20% greater to about 30% greater, about 20% greater to about 25% greater, about 25% greater to about 10,000% greater, about 25% greater to about 9,000% greater, about 25% greater to about 8,000% greater, about 25% greater to about 7,000% greater, about 25% greater to about 6,000% greater, about 25% greater to about 5,000% greater, about 25% greater to about 4,000% greater, about 25% greater to about 3,000% greater, about 25% greater to about 2,000% greater, about 25% greater to about 1,000% greater, about 25% greater to about 500% greater, about 25% greater to about 480% greater, about 25% greater to about 460% greater, about 25% greater to about 440% greater, about 25% greater to about 420% greater, about 25% greater to about 400% greater, about 25% greater to about 380% greater, about 25% greater to about 360% greater, about 25% greater to about 340% greater, about 25% greater to about 320% greater, about 25% greater to about 300% greater, about 25% greater to about 280% greater, about 25% greater to about 260% greater, about 25% greater to about 240% greater, about 25% greater to about 220% greater, about 25% greater to about 200% greater, about 25% greater to about 180% greater, about 25% greater to about 160% greater, about 25% greater to about 140% greater, about 25% greater to about 120% greater, about 25% greater to about 100% greater, about 25% greater to about 95% greater, about 25% greater to about 90% greater, about 25% greater to about 85% greater, about 25% greater to about 80% greater, about 25% greater to about 75% greater, about 25% greater to about 70% greater, about 25% greater to about 65% greater, about 25% greater to about 60% greater, about 25% greater to about 55% greater, about 25% greater to about 50% greater, about 25% greater to about 45% greater, about 25% greater to about 40% greater, about 25% greater to about 35% greater, about 25% greater to about 30% greater, about 30% greater to about 10,000% greater, about 30% greater to about 9,000% greater, about 30% greater to about 8,000% greater, about 30% greater to about 7,000% greater, about 30% greater to about 6,000% greater, about 30% greater to about 5,000% greater, about 30% greater to about 4,000% greater, about 30% greater to about 3,000% greater, about 30% greater to about 2,000% greater, about 30% greater to about 1,000% greater, about 30% greater to about 500% greater, about 30% greater to about 480% greater, about 30% greater to about 460% greater, about 30% greater to about 440% greater, about 30% greater to about 420% greater, about 30% greater to about 400% greater, about 30% greater to about 380% greater, about 30% greater to about 360% greater, about 30% greater to about 340% greater, about 30% greater to about 320% greater, about 30% greater to about 300% greater, about 30% greater to about 280% greater, about 30% greater to about 260% greater, about 30% greater to about 240% greater, about 30% greater to about 220% greater, about 30% greater to about 200% greater, about 30% greater to about 180% greater, about 30% greater to about 160% greater, about 30% greater to about 140% greater, about 30% greater to about 120% greater, about 30% greater to about 100% greater, about 30% greater to about 95% greater, about 30% greater to about 90% greater, about 30% greater to about 85% greater, about 30% greater to about 80% greater, about 30% greater to about 75% greater, about 30% greater to about 70% greater, about 30% greater to about 65% greater, about 30% greater to about 60% greater, about 30% greater to about 55% greater, about 30% greater to about 50% greater, about 30% greater to about 45% greater, about 30% greater to about 40% greater, about 30% greater to about 35% greater, about 35% greater to about 10,000% greater, about 35% greater to about 9,000% greater, about 35% greater to about 8,000% greater, about 35% greater to about 7,000% greater, about 35% greater to about 6,000% greater, about 35% greater to about 5,000% greater, about 35% greater to about 4,000% greater, about 35% greater to about 3,000% greater, about 35% greater to about 2,000% greater, about 35% greater to about 1,000% greater, about 35% greater to about 500% greater, about 35% greater to about 480% greater, about 35% greater to about 460% greater, about 35% greater to about 440% greater, about 35% greater to about 420% greater, about 35% greater to about 400% greater, about 35% greater to about 380% greater, about 35% greater to about 360% greater, about 35% greater to about 340% greater, about 35% greater to about 320% greater, about 35% greater to about 300% greater, about 35% greater to about 280% greater, about 35% greater to about 260% greater, about 35% greater to about 240% greater, about 35% greater to about 220% greater, about 35% greater to about 200% greater, about 35% greater to about 180% greater, about 35% greater to about 160% greater, about 35% greater to about 140% greater, about 35% greater to about 120% greater, about 35% greater to about 100% greater, about 35% greater to about 95% greater, about 35% greater to about 90% greater, about 35% greater to about 85% greater, about 35% greater to about 80% greater, about 35% greater to about 75% greater, about 35% greater to about 70% greater, about 35% greater to about 65% greater, about 35% greater to about 60% greater, about 35% greater to about 55% greater, about 35% greater to about 50% greater, about 35% greater to about 45% greater, about 35% greater to about 40% greater, about 40% greater to about 10,000% greater, about 40% greater to about 9,000% greater, about 40% greater to about 8,000% greater, about 40% greater to about 7,000% greater, about 40% greater to about 6,000% greater, about 40% greater to about 5,000% greater, about 40% greater to about 4,000% greater, about 40% greater to about 3,000% greater, about 40% greater to about 2,000% greater, about 40% greater to about 1,000% greater, about 40% greater to about 500% greater, about 40% greater to about 480% greater, about 40% greater to about 460% greater, about 40% greater to about 440% greater, about 40% greater to about 420% greater, about 40% greater to about 400% greater, about 40% greater to about 380% greater, about 40% greater to about 360% greater, about 40% greater to about 340% greater, about 40% greater to about 320% greater, about 40% greater to about 300% greater, about 40% greater to about 280% greater, about 40% greater to about 260% greater, about 40% greater to about 240% greater, about 40% greater to about 220% greater, about 40% greater to about 200% greater, about 40% greater to about 180% greater, about 40% greater to about 160% greater, about 40% greater to about 140% greater, about 40% greater to about 120% greater, about 40% greater to about 100% greater, about 40% greater to about 95% greater, about 40% greater to about 90% greater, about 40% greater to about 85% greater, about 40% greater to about 80% greater, about 40% greater to about 75% greater, about 40% greater to about 70% greater, about 40% greater to about 65% greater, about 40% greater to about 60% greater, about 40% greater to about 55% greater, about 40% greater to about 50% greater, about 40% greater to about 45% greater, about 45% greater to about 10,000% greater, about 45% greater to about 9,000% greater, about 45% greater to about 8,000% greater, about 45% greater to about 7,000% greater, about 45% greater to about 6,000% greater, about 45% greater to about 5,000% greater, about 45% greater to about 4,000% greater, about 45% greater to about 3,000% greater, about 45% greater to about 2,000% greater, about 45% greater to about 1,000% greater, about 45% greater to about 500% greater, about 45% greater to about 480% greater, about 45% greater to about 460% greater, about 45% greater to about 440% greater, about 45% greater to about 420% greater, about 45% greater to about 400% greater, about 45% greater to about 380% greater, about 45% greater to about 360% greater, about 45% greater to about 340% greater, about 45% greater to about 320% greater, about 45% greater to about 300% greater, about 45% greater to about 280% greater, about 45% greater to about 260% greater, about 45% greater to about 240% greater, about 45% greater to about 220% greater, about 45% greater to about 200% greater, about 45% greater to about 180% greater, about 45% greater to about 160% greater, about 45% greater to about 140% greater, about 45% greater to about 120% greater, about 45% greater to about 100% greater, about 45% greater to about 95% greater, about 45% greater to about 90% greater, about 45% greater to about 85% greater, about 45% greater to about 80% greater, about 45% greater to about 75% greater, about 45% greater to about 70% greater, about 45% greater to about 65% greater, about 45% greater to about 60% greater, about 45% greater to about 55% greater, about 45% greater to about 50% greater, about 50% greater to about 10,000% greater, about 50% greater to about 9,000% greater, about 50% greater to about 8,000% greater, about 50% greater to about 7,000% greater, about 50% greater to about 6,000% greater, about 50% greater to about 5,000% greater, about 50% greater to about 4,000% greater, about 50% greater to about 3,000% greater, about 50% greater to about 2,000% greater, about 50% greater to about 1,000% greater, about 50% greater to about 500% greater, about 50% greater to about 480% greater, about 50% greater to about 460% greater, about 50% greater to about 440% greater, about 50% greater to about 420% greater, about 50% greater to about 400% greater, about 50% greater to about 380% greater, about 50% greater to about 360% greater, about 50% greater to about 340% greater, about 50% greater to about 320% greater, about 50% greater to about 300% greater, about 50% greater to about 280% greater, about 50% greater to about 260% greater, about 50% greater to about 240% greater, about 50% greater to about 220% greater, about 50% greater to about 200% greater, about 50% greater to about 180% greater, about 50% greater to about 160% greater, about 50% greater to about 140% greater, about 50% greater to about 120% greater, about 50% greater to about 100% greater, about 50% greater to about 95% greater, about 50% greater to about 90% greater, about 50% greater to about 85% greater, about 50% greater to about 80% greater, about 50% greater to about 75% greater, about 50% greater to about 70% greater, about 50% greater to about 65% greater, about 50% greater to about 60% greater, about 50% greater to about 55% greater, about 55% greater to about 10,000% greater, about 55% greater to about 9,000% greater, about 55% greater to about 8,000% greater, about 55% greater to about 7,000% greater, about 55% greater to about 6,000% greater, about 55% greater to about 5,000% greater, about 55% greater to about 4,000% greater, about 55% greater to about 3,000% greater, about 55% greater to about 2,000% greater, about 55% greater to about 1,000% greater, about 55% greater to about 500% greater, about 55% greater to about 480% greater, about 55% greater to about 460% greater, about 55% greater to about 440% greater, about 55% greater to about 420% greater, about 55% greater to about 400% greater, about 55% greater to about 380% greater, about 55% greater to about 360% greater, about 55% greater to about 340% greater, about 55% greater to about 320% greater, about 55% greater to about 300% greater, about 55% greater to about 280% greater, about 55% greater to about 260% greater, about 55% greater to about 240% greater, about 55% greater to about 220% greater, about 55% greater to about 200% greater, about 55% greater to about 180% greater, about 55% greater to about 160% greater, about 55% greater to about 140% greater, about 55% greater to about 120% greater, about 55% greater to about 100% greater, about 55% greater to about 95% greater, about 55% greater to about 90% greater, about 55% greater to about 85% greater, about 55% greater to about 80% greater, about 55% greater to about 75% greater, about 55% greater to about 70% greater, about 55% greater to about 65% greater, about 55% greater to about 60% greater, about 60% greater to about 10,000% greater, about 60% greater to about 9,000% greater, about 60% greater to about 8,000% greater, about 60% greater to about 7,000% greater, about 60% greater to about 6,000% greater, about 60% greater to about 5,000% greater, about 60% greater to about 4,000% greater, about 60% greater to about 3,000% greater, about 60% greater to about 2,000% greater, about 60% greater to about 1,000% greater, about 60% greater to about 500% greater, about 60% greater to about 480% greater, about 60% greater to about 460% greater, about 60% greater to about 440% greater, about 60% greater to about 420% greater, about 60% greater to about 400% greater, about 60% greater to about 380% greater, about 60% greater to about 360% greater, about 60% greater to about 340% greater, about 60% greater to about 320% greater, about 60% greater to about 300% greater, about 60% greater to about 280% greater, about 60% greater to about 260% greater, about 60% greater to about 240% greater, about 60% greater to about 220% greater, about 60% greater to about 200% greater, about 60% greater to about 180% greater, about 60% greater to about 160% greater, about 60% greater to about 140% greater, about 60% greater to about 120% greater, about 60% greater to about 100% greater, about 60% greater to about 95% greater, about 60% greater to about 90% greater, about 60% greater to about 85% greater, about 60% greater to about 80% greater, about 60% greater to about 75% greater, about 60% greater to about 70% greater, about 60% greater to about 65% greater, about 65% greater to about 10,000% greater, about 65% greater to about 9,000% greater, about 65% greater to about 8,000% greater, about 65% greater to about 7,000% greater, about 65% greater to about 6,000% greater, about 65% greater to about 5,000% greater, about 65% greater to about 4,000% greater, about 65% greater to about 3,000% greater, about 65% greater to about 2,000% greater, about 65% greater to about 1,000% greater, about 65% greater to about 500% greater, about 65% greater to about 480% greater, about 65% greater to about 460% greater, about 65% greater to about 440% greater, about 65% greater to about 420% greater, about 65% greater to about 400% greater, about 65% greater to about 380% greater, about 65% greater to about 360% greater, about 65% greater to about 340% greater, about 65% greater to about 320% greater, about 65% greater to about 300% greater, about 65% greater to about 280% greater, about 65% greater to about 260% greater, about 65% greater to about 240% greater, about 65% greater to about 220% greater, about 65% greater to about 200% greater, about 65% greater to about 180% greater, about 65% greater to about 160% greater, about 65% greater to about 140% greater, about 65% greater to about 120% greater, about 65% greater to about 100% greater, about 65% greater to about 95% greater, about 65% greater to about 90% greater, about 65% greater to about 85% greater, about 65% greater to about 80% greater, about 65% greater to about 75% greater, about 65% greater to about 70% greater, about 70% greater to about 10,000% greater, about 70% greater to about 9,000% greater, about 70% greater to about 8,000% greater, about 70% greater to about 7,000% greater, about 70% greater to about 6,000% greater, about 70% greater to about 5,000% greater, about 70% greater to about 4,000% greater, about 70% greater to about 3,000% greater, about 70% greater to about 2,000% greater, about 70% greater to about 1,000% greater, about 70% greater to about 500% greater, about 70% greater to about 480% greater, about 70% greater to about 460% greater, about 70% greater to about 440% greater, about 70% greater to about 420% greater, about 70% greater to about 400% greater, about 70% greater to about 380% greater, about 70% greater to about 360% greater, about 70% greater to about 340% greater, about 70% greater to about 320% greater, about 70% greater to about 300% greater, about 70% greater to about 280% greater, about 70% greater to about 260% greater, about 70% greater to about 240% greater, about 70% greater to about 220% greater, about 70% greater to about 200% greater, about 70% greater to about 180% greater, about 70% greater to about 160% greater, about 70% greater to about 140% greater, about 70% greater to about 120% greater, about 70% greater to about 100% greater, about 70% greater to about 95% greater, about 70% greater to about 90% greater, about 70% greater to about 85% greater, about 70% greater to about 80% greater, about 70% greater to about 75% greater, about 75% greater to about 10,000% greater, about 75% greater to about 9,000% greater, about 75% greater to about 8,000% greater, about 75% greater to about 7,000% greater, about 75% greater to about 6,000% greater, about 75% greater to about 5,000% greater, about 75% greater to about 4,000% greater, about 75% greater to about 3,000% greater, about 75% greater to about 2,000% greater, about 75% greater to about 1,000% greater, about 75% greater to about 500% greater, about 75% greater to about 480% greater, about 75% greater to about 460% greater, about 75% greater to about 440% greater, about 75% greater to about 420% greater, about 75% greater to about 400% greater, about 75% greater to about 380% greater, about 75% greater to about 360% greater, about 75% greater to about 340% greater, about 75% greater to about 320% greater, about 75% greater to about 300% greater, about 75% greater to about 280% greater, about 75% greater to about 260% greater, about 75% greater to about 240% greater, about 75% greater to about 220% greater, about 75% greater to about 200% greater, about 75% greater to about 180% greater, about 75% greater to about 160% greater, about 75% greater to about 140% greater, about 75% greater to about 120% greater, about 75% greater to about 100% greater, about 75% greater to about 95% greater, about 75% greater to about 90% greater, about 75% greater to about 85% greater, about 75% greater to about 80% greater, about 80% greater to about 10,000% greater, about 80% greater to about 9,000% greater, about 80% greater to about 8,000% greater, about 80% greater to about 7,000% greater, about 80% greater to about 6,000% greater, about 80% greater to about 5,000% greater, about 80% greater to about 4,000% greater, about 80% greater to about 3,000% greater, about 80% greater to about 2,000% greater, about 80% greater to about 1,000% greater, about 80% greater to about 500% greater, about 80% greater to about 480% greater, about 80% greater to about 460% greater, about 80% greater to about 440% greater, about 80% greater to about 420% greater, about 80% greater to about 400% greater, about 80% greater to about 380% greater, about 80% greater to about 360% greater, about 80% greater to about 340% greater, about 80% greater to about 320% greater, about 80% greater to about 300% greater, about 80% greater to about 280% greater, about 80% greater to about 260% greater, about 80% greater to about 240% greater, about 80% greater to about 220% greater, about 80% greater to about 200% greater, about 80% greater to about 180% greater, about 80% greater to about 160% greater, about 80% greater to about 140% greater, about 80% greater to about 120% greater, about 80% greater to about 100% greater, about 80% greater to about 95% greater, about 80% greater to about 90% greater, about 80% greater to about 85% greater, about 85% greater to about 10,000% greater, about 85% greater to about 9,000% greater, about 85% greater to about 8,000% greater, about 85% greater to about 7,000% greater, about 85% greater to about 6,000% greater, about 85% greater to about 5,000% greater, about 85% greater to about 4,000% greater, about 85% greater to about 3,000% greater, about 85% greater to about 2,000% greater, about 85% greater to about 1,000% greater, about 85% greater to about 500% greater, about 85% greater to about 480% greater, about 85% greater to about 460% greater, about 85% greater to about 440% greater, about 85% greater to about 420% greater, about 85% greater to about 400% greater, about 85% greater to about 380% greater, about 85% greater to about 360% greater, about 85% greater to about 340% greater, about 85% greater to about 320% greater, about 85% greater to about 300% greater, about 85% greater to about 280% greater, about 85% greater to about 260% greater, about 85% greater to about 240% greater, about 85% greater to about 220% greater, about 85% greater to about 200% greater, about 85% greater to about 180% greater, about 85% greater to about 160% greater, about 85% greater to about 140% greater, about 85% greater to about 120% greater, about 85% greater to about 100% greater, about 85% greater to about 95% greater, about 85% greater to about 90% greater, about 90% greater to about 10,000% greater, about 90% greater to about 9,000% greater, about 90% greater to about 8,000% greater, about 90% greater to about 7,000% greater, about 90% greater to about 6,000% greater, about 90% greater to about 5,000% greater, about 90% greater to about 4,000% greater, about 90% greater to about 3,000% greater, about 90% greater to about 2,000% greater, about 90% greater to about 1,000% greater, about 90% greater to about 500% greater, about 90% greater to about 480% greater, about 90% greater to about 460% greater, about 90% greater to about 440% greater, about 90% greater to about 420% greater, about 90% greater to about 400% greater, about 90% greater to about 380% greater, about 90% greater to about 360% greater, about 90% greater to about 340% greater, about 90% greater to about 320% greater, about 90% greater to about 300% greater, about 90% greater to about 280% greater, about 90% greater to about 260% greater, about 90% greater to about 240% greater, about 90% greater to about 220% greater, about 90% greater to about 200% greater, about 90% greater to about 180% greater, about 90% greater to about 160% greater, about 90% greater to about 140% greater, about 90% greater to about 120% greater, about 90% greater to about 100% greater, about 90% greater to about 95% greater, about 95% greater to about 10,000% greater, about 95% greater to about 9,000% greater, about 95% greater to about 8,000% greater, about 95% greater to about 7,000% greater, about 95% greater to about 6,000% greater, about 95% greater to about 5,000% greater, about 95% greater to about 4,000% greater, about 95% greater to about 3,000% greater, about 95% greater to about 2,000% greater, about 95% greater to about 1,000% greater, about 95% greater to about 500% greater, about 95% greater to about 480% greater, about 95% greater to about 460% greater, about 95% greater to about 440% greater, about 95% greater to about 420% greater, about 95% greater to about 400% greater, about 95% greater to about 380% greater, about 95% greater to about 360% greater, about 95% greater to about 340% greater, about 95% greater to about 320% greater, about 95% greater to about 300% greater, about 95% greater to about 280% greater, about 95% greater to about 260% greater, about 95% greater to about 240% greater, about 95% greater to about 220% greater, about 95% greater to about 200% greater, about 95% greater to about 180% greater, about 95% greater to about 160% greater, about 95% greater to about 140% greater, about 95% greater to about 120% greater, about 95% greater to about 100% greater, about 100% greater to about 10,000% greater, about 100% greater to about 9,000% greater, about 100% greater to about 8,000% greater, about 100% greater to about 7,000% greater, about 100% greater to about 6,000% greater, about 100% greater to about 5,000% greater, about 100% greater to about 4,000% greater, about 100% greater to about 3,000% greater, about 100% greater to about 2,000% greater, about 100% greater to about 1,000% greater, about 100% greater to about 500% greater, about 100% greater to about 480% greater, about 100% greater to about 460% greater, about 100% greater to about 440% greater, about 100% greater to about 420% greater, about 100% greater to about 400% greater, about 100% greater to about 380% greater, about 100% greater to about 360% greater, about 100% greater to about 340% greater, about 100% greater to about 320% greater, about 100% greater to about 300% greater, about 100% greater to about 280% greater, about 100% greater to about 260% greater, about 100% greater to about 240% greater, about 100% greater to about 220% greater, about 100% greater to about 200% greater, about 100% greater to about 180% greater, about 100% greater to about 160% greater, about 100% greater to about 140% greater, about 100% greater to about 120% greater, about 120% greater to about 10,000% greater, about 120% greater to about 9,000% greater, about 120% greater to about 8,000% greater, about 120% greater to about 7,000% greater, about 120% greater to about 6,000% greater, about 120% greater to about 5,000% greater, about 120% greater to about 4,000% greater, about 120% greater to about 3,000% greater, about 120% greater to about 2,000% greater, about 120% greater to about 1,000% greater, about 120% greater to about 500% greater, about 120% greater to about 480% greater, about 120% greater to about 460% greater, about 120% greater to about 440% greater, about 120% greater to about 420% greater, about 120% greater to about 400% greater, about 120% greater to about 380% greater, about 120% greater to about 360% greater, about 120% greater to about 340% greater, about 120% greater to about 320% greater, about 120% greater to about 300% greater, about 120% greater to about 280% greater, about 120% greater to about 260% greater, about 120% greater to about 240% greater, about 120% greater to about 220% greater, about 120% greater to about 200% greater, about 120% greater to about 180% greater, about 120% greater to about 160% greater, about 120% greater to about 140% greater, about 140% greater to about 10,000% greater, about 140% greater to about 9,000% greater, about 140% greater to about 8,000% greater, about 140% greater to about 7,000% greater, about 140% greater to about 6,000% greater, about 140% greater to about 5,000% greater, about 140% greater to about 4,000% greater, about 140% greater to about 3,000% greater, about 140% greater to about 2,000% greater, about 140% greater to about 1,000% greater, about 140% greater to about 500% greater, about 140% greater to about 480% greater, about 140% greater to about 460% greater, about 140% greater to about 440% greater, about 140% greater to about 420% greater, about 140% greater to about 400% greater, about 140% greater to about 380% greater, about 140% greater to about 360% greater, about 140% greater to about 340% greater, about 140% greater to about 320% greater, about 140% greater to about 300% greater, about 140% greater to about 280% greater, about 140% greater to about 260% greater, about 140% greater to about 240% greater, about 140% greater to about 220% greater, about 140% greater to about 200% greater, about 140% greater to about 180% greater, about 140% greater to about 160% greater, about 160% greater to about 10,000% greater, about 160% greater to about 9,000% greater, about 160% greater to about 8,000% greater, about 160% greater to about 7,000% greater, about 160% greater to about 6,000% greater, about 160% greater to about 5,000% greater, about 160% greater to about 4,000% greater, about 160% greater to about 3,000% greater, about 160% greater to about 2,000% greater, about 160% greater to about 1,000% greater, about 160% greater to about 500% greater, about 160% greater to about 480% greater, about 160% greater to about 460% greater, about 160% greater to about 440% greater, about 160% greater to about 420% greater, about 160% greater to about 400% greater, about 160% greater to about 380% greater, about 160% greater to about 360% greater, about 160% greater to about 340% greater, about 160% greater to about 320% greater, about 160% greater to about 300% greater, about 160% greater to about 280% greater, about 160% greater to about 260% greater, about 160% greater to about 240% greater, about 160% greater to about 220% greater, about 160% greater to about 200% greater, about 160% greater to about 180% greater, about 180% greater to about 10,000% greater, about 180% greater to about 9,000% greater, about 180% greater to about 8,000% greater, about 180% greater to about 7,000% greater, about 180% greater to about 6,000% greater, about 180% greater to about 5,000% greater, about 180% greater to about 4,000% greater, about 180% greater to about 3,000% greater, about 180% greater to about 2,000% greater, about 180% greater to about 1,000% greater, about 180% greater to about 500% greater, about 180% greater to about 480% greater, about 180% greater to about 460% greater, about 180% greater to about 440% greater, about 180% greater to about 420% greater, about 180% greater to about 400% greater, about 180% greater to about 380% greater, about 180% greater to about 360% greater, about 180% greater to about 340% greater, about 180% greater to about 320% greater, about 180% greater to about 300% greater, about 180% greater to about 280% greater, about 180% greater to about 260% greater, about 180% greater to about 240% greater, about 180% greater to about 220% greater, about 180% greater to about 200% greater, about 200% greater to about 10,000% greater, about 200% greater to about 9,000% greater, about 200% greater to about 8,000% greater, about 200% greater to about 7,000% greater, about 200% greater to about 6,000% greater, about 200% greater to about 5,000% greater, about 200% greater to about 4,000% greater, about 200% greater to about 3,000% greater, about 200% greater to about 2,000% greater, about 200% greater to about 1,000% greater, about 200% greater to about 500% greater, about 200% greater to about 480% greater, about 200% greater to about 460% greater, about 200% greater to about 440% greater, about 200% greater to about 420% greater, about 200% greater to about 400% greater, about 200% greater to about 380% greater, about 200% greater to about 360% greater, about 200% greater to about 340% greater, about 200% greater to about 320% greater, about 200% greater to about 300% greater, about 200% greater to about 280% greater, about 200% greater to about 260% greater, about 200% greater to about 240% greater, about 200% greater to about 220% greater, about 220% greater to about 10,000% greater, about 220% greater to about 9,000% greater, about 220% greater to about 8,000% greater, about 220% greater to about 7,000% greater, about 220% greater to about 6,000% greater, about 220% greater to about 5,000% greater, about 220% greater to about 4,000% greater, about 220% greater to about 3,000% greater, about 220% greater to about 2,000% greater, about 220% greater to about 1,000% greater, about 220% greater to about 500% greater, about 220% greater to about 480% greater, about 220% greater to about 460% greater, about 220% greater to about 440% greater, about 220% greater to about 420% greater, about 220% greater to about 400% greater, about 220% greater to about 380% greater, about 220% greater to about 360% greater, about 220% greater to about 340% greater, about 220% greater to about 320% greater, about 220% greater to about 300% greater, about 220% greater to about 280% greater, about 220% greater to about 260% greater, about 220% greater to about 240% greater, about 240% greater to about 10,000% greater, about 240% greater to about 9,000% greater, about 240% greater to about 8,000% greater, about 240% greater to about 7,000% greater, about 240% greater to about 6,000% greater, about 240% greater to about 5,000% greater, about 240% greater to about 4,000% greater, about 240% greater to about 3,000% greater, about 240% greater to about 2,000% greater, about 240% greater to about 1,000% greater, about 240% greater to about 500% greater, about 240% greater to about 480% greater, about 240% greater to about 460% greater, about 240% greater to about 440% greater, about 240% greater to about 420% greater, about 240% greater to about 400% greater, about 240% greater to about 380% greater, about 240% greater to about 360% greater, about 240% greater to about 340% greater, about 240% greater to about 320% greater, about 240% greater to about 300% greater, about 240% greater to about 280% greater, about 240% greater to about 260% greater, about 260% greater to about 10,000% greater, about 260% greater to about 9,000% greater, about 260% greater to about 8,000% greater, about 260% greater to about 7,000% greater, about 260% greater to about 6,000% greater, about 260% greater to about 5,000% greater, about 260% greater to about 4,000% greater, about 260% greater to about 3,000% greater, about 260% greater to about 2,000% greater, about 260% greater to about 1,000% greater, about 260% greater to about 500% greater, about 260% greater to about 480% greater, about 260% greater to about 460% greater, about 260% greater to about 440% greater, about 260% greater to about 420% greater, about 260% greater to about 400% greater, about 260% greater to about 380% greater, about 260% greater to about 360% greater, about 260% greater to about 340% greater, about 260% greater to about 320% greater, about 260% greater to about 300% greater, about 260% greater to about 280% greater, about 280% greater to about 10,000% greater, about 280% greater to about 9,000% greater, about 280% greater to about 8,000% greater, about 280% greater to about 7,000% greater, about 280% greater to about 6,000% greater, about 280% greater to about 5,000% greater, about 280% greater to about 4,000% greater, about 280% greater to about 3,000% greater, about 280% greater to about 2,000% greater, about 280% greater to about 1,000% greater, about 280% greater to about 500% greater, about 280% greater to about 480% greater, about 280% greater to about 460% greater, about 280% greater to about 440% greater, about 280% greater to about 420% greater, about 280% greater to about 400% greater, about 280% greater to about 380% greater, about 280% greater to about 360% greater, about 280% greater to about 340% greater, about 280% greater to about 320% greater, about 280% greater to about 300% greater, about 300% greater to about 10,000% greater, about 300% greater to about 9,000% greater, about 300% greater to about 8,000% greater, about 300% greater to about 7,000% greater, about 300% greater to about 6,000% greater, about 300% greater to about 5,000% greater, about 300% greater to about 4,000% greater, about 300% greater to about 3,000% greater, about 300% greater to about 2,000% greater, about 300% greater to about 1,000% greater, about 300% greater to about 500% greater, about 300% greater to about 480% greater, about 300% greater to about 460% greater, about 300% greater to about 440% greater, about 300% greater to about 420% greater, about 300% greater to about 400% greater, about 300% greater to about 380% greater, about 300% greater to about 360% greater, about 300% greater to about 340% greater, about 300% greater to about 320% greater, about 320% greater to about 10,000% greater, about 320% greater to about 9,000% greater, about 320% greater to about 8,000% greater, about 320% greater to about 7,000% greater, about 320% greater to about 6,000% greater, about 320% greater to about 5,000% greater, about 320% greater to about 4,000% greater, about 320% greater to about 3,000% greater, about 320% greater to about 2,000% greater, about 320% greater to about 1,000% greater, about 320% greater to about 500% greater, about 320% greater to about 480% greater, about 320% greater to about 460% greater, about 320% greater to about 440% greater, about 320% greater to about 420% greater, about 320% greater to about 400% greater, about 320% greater to about 380% greater, about 320% greater to about 360% greater, about 320% greater to about 340% greater, about 340% greater to about 10,000% greater, about 340% greater to about 9,000% greater, about 340% greater to about 8,000% greater, about 340% greater to about 7,000% greater, about 340% greater to about 6,000% greater, about 340% greater to about 5,000% greater, about 340% greater to about 4,000% greater, about 340% greater to about 3,000% greater, about 340% greater to about 2,000% greater, about 340% greater to about 1,000% greater, about 340% greater to about 500% greater, about 340% greater to about 480% greater, about 340% greater to about 460% greater, about 340% greater to about 440% greater, about 340% greater to about 420% greater, about 340% greater to about 400% greater, about 340% greater to about 380% greater, about 340% greater to about 360% greater, about 360% greater to about 10,000% greater, about 360% greater to about 9,000% greater, about 360% greater to about 8,000% greater, about 360% greater to about 7,000% greater, about 360% greater to about 6,000% greater, about 360% greater to about 5,000% greater, about 360% greater to about 4,000% greater, about 360% greater to about 3,000% greater, about 360% greater to about 2,000% greater, about 360% greater to about 1,000% greater, about 360% greater to about 500% greater, about 360% greater to about 480% greater, about 360% greater to about 460% greater, about 360% greater to about 440% greater, about 360% greater to about 420% greater, about 360% greater to about 400% greater, about 360% greater to about 380% greater, about 380% greater to about 10,000% greater, about 380% greater to about 9,000% greater, about 380% greater to about 8,000% greater, about 380% greater to about 7,000% greater, about 380% greater to about 6,000% greater, about 380% greater to about 5,000% greater, about 380% greater to about 4,000% greater, about 380% greater to about 3,000% greater, about 380% greater to about 2,000% greater, about 380% greater to about 1,000% greater, about 380% greater to about 500% greater, about 380% greater to about 480% greater, about 380% greater to about 460% greater, about 380% greater to about 440% greater, about 380% greater to about 420% greater, about 380% greater to about 400% greater, about 400% greater to about 10,000% greater, about 400% greater to about 9,000% greater, about 400% greater to about 8,000% greater, about 400% greater to about 7,000% greater, about 400% greater to about 6,000% greater, about 400% greater to about 5,000% greater, about 400% greater to about 4,000% greater, about 400% greater to about 3,000% greater, about 400% greater to about 2,000% greater, about 400% greater to about 1,000% greater, about 400% greater to about 500% greater, about 400% greater to about 480% greater, about 400% greater to about 460% greater, about 400% greater to about 440% greater, about 400% greater to about 420% greater, about 420% greater to about 10,000% greater, about 420% greater to about 9,000% greater, about 420% greater to about 8,000% greater, about 420% greater to about 7,000% greater, about 420% greater to about 6,000% greater, about 420% greater to about 5,000% greater, about 420% greater to about 4,000% greater, about 420% greater to about 3,000% greater, about 420% greater to about 2,000% greater, about 420% greater to about 1,000% greater, about 420% greater to about 500% greater, about 420% greater to about 480% greater, about 420% greater to about 460% greater, about 420% greater to about 440% greater, about 440% greater to about 10,000% greater, about 440% greater to about 9,000% greater, about 440% greater to about 8,000% greater, about 440% greater to about 7,000% greater, about 440% greater to about 6,000% greater, about 440% greater to about 5,000% greater, about 440% greater to about 4,000% greater, about 440% greater to about 3,000% greater, about 440% greater to about 2,000% greater, about 440% greater to about 1,000% greater, about 440% greater to about 500% greater, about 440% greater to about 480% greater, about 440% greater to about 460% greater, about 460% greater to about 10,000% greater, about 460% greater to about 9,000% greater, about 460% greater to about 8,000% greater, about 460% greater to about 7,000% greater, about 460% greater to about 6,000% greater, about 460% greater to about 5,000% greater, about 460% greater to about 4,000% greater, about 460% greater to about 3,000% greater, about 460% greater to about 2,000% greater, about 460% greater to about 1,000% greater, about 460% greater to about 500% greater, about 460% greater to about 480% greater, about 480% greater to about 10,000% greater, about 480% greater to about 9,000% greater, about 480% greater to about 8,000% greater, about 480% greater to about 7,000% greater, about 480% greater to about 6,000% greater, about 480% greater to about 5,000% greater, about 480% greater to about 4,000% greater, about 480% greater to about 3,000% greater, about 480% greater to about 2,000% greater, about 480% greater to about 1,000% greater, about 480% greater to about 500% greater, about 500% greater to about 10,000% greater, about 500% greater to about 9,000% greater, about 500% greater to about 8,000% greater, about 500% greater to about 7,000% greater, about 500% greater to about 6,000% greater, about 500% greater to about 5,000% greater, about 500% greater to about 4,000% greater, about 500% greater to about 3,000% greater, about 500% greater to about 2,000% greater, about 500% greater to about 1,000% greater, about 1,000% greater to about 10,000% greater, about 1,000% greater to about 9,000% greater, about 1,000% greater to about 8,000% greater, about 1,000% greater to about 7,000% greater, about 1,000% greater to about 6,000% greater, about 1,000% greater to about 5,000% greater, about 1,000% greater to about 4,000% greater, about 1,000% greater to about 3,000% greater, about 1,000% greater to about 2,000% greater, about 2,000% greater to about 10,000% greater, about 2,000% greater to about 9,000% greater, about 2,000% greater to about 8,000% greater, about 2,000% greater to about 7,000% greater, about 2,000% greater to about 6,000% greater, about 2,000% greater to about 5,000% greater, about 2,000% greater to about 4,000% greater, about 2,000% greater to about 3,000% greater, about 3,000% greater to about 10,000% greater, about 3,000% greater to about 9,000% greater, about 3,000% greater to about 8,000% greater, about 3,000% greater to about 7,000% greater, about 3,000% greater to about 6,000% greater, about 3,000% greater to about 5,000% greater, about 3,000% greater to about 4,000% greater, about 4,000% greater to about 10,000% greater, about 4,000% greater to about 9,000% greater, about 4,000% greater to about 8,000% greater, about 4,000% greater to about 7,000% greater, about 4,000% greater to about 6,000% greater, about 4,000% greater to about 5,000% greater, about 5,000% greater to about 10,000% greater, about 5,000% greater to about 9,000% greater, about 5,000% greater to about 8,000% greater, about 5,000% greater to about 7,000% greater, about 5,000% greater to about 6,000% greater, about 6,000% greater to about 10,000% greater, about 6,000% greater to about 9,000% greater, about 6,000% greater to about 8,000% greater, about 6,000% greater to about 7,000% greater, about 7,000% greater to about 10,000% greater, about 7,000% greater to about 9,000% greater, about 7,000% greater to about 8,000% greater, about 8,000% greater to about 10,000% greater, about 8,000% greater to about 9,000% greater, or about 9,000% greater to about 10,000% greater) than the K_{D} at a pH of about 7.0 to about 8.0 (e.g., any of the subranges of this range described herein).

In some embodiments of any of the antigen-binding protein constructs (ABPCs) described herein, the dissociation rate of the first antigen-binding domain (and optionally the second antigen-binding domain, if present) at a pH of about 4.0 to about 6.5 (e.g., any of the subranges of this range described herein) is faster (e.g., at least 0.2-fold faster, at least 0.3-fold, at least 0.4-fold, at least 0.5-fold, at least 0.6-fold, at least 0.7-fold, at least 0.8-fold, at least 0.9-fold, at least 1.0-fold, at least 1.5-fold, at least 2.0-fold, at least 2.5-fold, at least 3.0 fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 10.5-fold, at least 11.0-fold, at least 11.5-fold, at least 12.0-fold, at least 12.5-fold, at least 13.0-fold, at least 13.5-fold, at least 14.0-fold, at least 14.5-fold, at least 15.0-fold, at least 15.5-fold, at least 16.0-fold, at least 16.5-fold, at least 17.0-fold, at least 17.5-fold, at least 18.0-fold, at least 18.5-fold, at least 19.0-fold, at least 19.5-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, or at least 100-fold faster or about 0.2-fold to about 100-fold faster, about 0.2-fold to about 90-fold faster, about 0.2-fold to about 80-fold faster, about 0.2-fold to about 70-fold faster, about 0.2-fold to about 60-fold faster, about 0.2-fold to about 50-fold faster, about 0.2-fold to about 40-fold faster, about 0.2-fold to about 30-fold faster, about 0.2-fold to about 20-fold faster, about 0.2-fold to about 15-fold faster, about 0.2-fold to about 10-fold faster, about 0.2-fold to about 5-fold, about 0.2-fold to about 2-fold faster, about 0.2-fold to about 1-fold faster, about 0.2-fold to about 0.5-fold faster, about 0.5-fold to about 100-fold faster, about 0.5-fold to about 90-fold faster, about 0.5-fold to about 80-fold faster, about 0.5-fold to about 70-fold faster, about 0.5-fold to about 60-fold faster, about 0.5-fold to about 50-fold faster, about 0.5-fold to about 40-fold faster, about 0.5-fold to about 30-fold faster, about 0.5-fold to about 20-fold faster, about 0.5-fold to about 15-fold faster, about 0.5-fold to about 10-fold faster, about 0.5-fold to about 5-fold, about 0.5-fold to about 2-fold faster, about 0.5-fold to about 1-fold faster, about 1-fold to about 100-fold faster, about 1-fold to about 90-fold faster, about 1-fold to about 80-fold faster, about 1-fold to about 70-fold faster, about 1-fold to about 60-fold faster, about 1-fold to about 50-fold faster, about 1-fold to about 40-fold faster, about 1-fold to about 30-fold faster, about 1-fold to about 20-fold faster, about 1-fold to about 15-fold faster, about 1-fold to about 10-fold faster, about 1-fold to about 5-fold, about 1-fold to about 2-fold faster, about 2-fold to about 100-fold faster, about 2-fold to about 90-fold faster, about 2-fold to about 80-fold faster, about 2-fold to about 70-fold faster, about 2-fold to about 60-fold faster, about 2-fold to about 50-fold faster, about 2-fold to about 40-fold faster, about 2-fold to about 30-fold faster, about 2-fold to about 20-fold faster, about 2-fold to about 15-fold faster, about 2-fold to about 10-fold faster, about 2-fold to about 5-fold, about 5-fold to about 100-fold faster, about 5-fold to about 90-fold faster, about 5-fold to about 80-fold faster, about 5-fold to about 70-fold faster, about 5-fold to about 60-fold faster, about 5-fold to about 50-fold faster, about 5-fold to about 40-fold faster, about 5-fold to about 30-fold faster, about 5-fold to about 20-fold faster, about 5-fold to about 15-fold faster, about 5-fold to about 10-fold faster, about 10-fold to about 100-fold faster, about 10-fold to about 90-fold faster, about 10-fold to about 80-fold faster, about 10-fold to about 70-fold faster, about 10-fold to about 60-fold faster, about 10-fold to about 50-fold faster, about 10-fold to about 40-fold faster, about 10-fold to about 30-fold faster, about 10-fold to about 20-fold faster, about 10-fold to about 15-fold faster, about 15-fold to about 100-fold faster, about 15-fold to about 90-fold faster, about 15-fold to about 80-fold faster, about 15-fold to about 70-fold faster, about 15-fold to about 60-fold faster, about 15-fold to about 50-fold faster, about 15-fold to about 40-fold faster, about 15-fold to about 30-fold faster, about 15-fold to about 20-fold faster, about 20-fold to about 100-fold faster, about 20-fold to about 90-fold faster, about 20-fold to about 80-fold faster, about 20-fold to about 70-fold faster, about 20-fold to about 60-fold faster, about 20-fold to about 50-fold faster, about 20-fold to about 40-fold faster, about 20-fold to about 30-fold faster, about 30-fold to about 100-fold faster, about 30-fold to about 90-fold faster, about 30-fold to about 80-fold faster, about 30-fold to about 70-fold faster, about 30-fold to about 60-fold faster, about 30-fold to about 50-fold faster, about 30-fold to about 40-fold faster, about 40-fold to about 100-fold faster, about 40-fold to about 90-fold faster, about 40-fold to about 80-fold faster, about 40-fold to about 70-fold faster, about 40-fold to about 60-fold faster, about 40-fold to about 50-fold faster, about 50-fold to about 100-fold faster, about 50-fold to about 90-fold faster, about 50-fold to about 80-fold faster, about 50-fold to about 70-fold faster, about 50-fold to about 60-fold faster, about 60-fold to about 100-fold faster, about 60-fold to about 90-fold faster, about 60-fold to about 80-fold faster, about 60-fold to about 70-fold faster, about 70-fold to about 100-fold faster, about 70-fold to about 90-fold faster, about 70-fold to about 80-fold faster, about 80-fold to about 100-fold faster, about 80-fold to about 90-fold faster, or about 90-fold to about 100-fold faster) than the dissociation rate at a pH of about 7.0 to about 8.0 (e.g., or any of the subranges of this range described herein).

In some embodiments of any of the antigen-binding protein constructs (ABPCs) described herein, the dissociation constant (K_{D}) of the first antigen-binding domain (and optionally the second antigen-binding domain, if present) at a pH of about 4.0 to about 6.5 (e.g., any of the subranges of this range described herein) is greater (e.g., detectably greater) (e.g., at least 0.2-fold greater, at least 0.3-fold, at least 0.4-fold, at least 0.5-fold, at least 0.6-fold, at least 0.7-fold, at least 0.8-fold, at least 0.9-fold, at least 1.0-fold, at least 1.5-fold, at least 2.0-fold, at least 2.5-fold, at least 3.0 fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0-fold, at least 10.5-fold, at least 11.0-fold, at least 11.5-fold, at least 12.0-fold, at least 12.5-fold, at least 13.0-fold, at least 13.5-fold, at least 14.0-fold, at least 14.5-fold, at least 15.0-fold, at least 15.5-fold, at least 16.0-fold, at least 16.5-fold, at least 17.0-fold, at least 17.5-fold, at least 18.0-fold, at least 18.5-fold, at least 19.0-fold, at least 19.5-fold, at least 20-fold greater, at least 25-fold greater, at least 30-fold greater, at least 35-fold greater, at least 40-fold greater, at least 45-fold greater, at least 50-fold greater, at least 55-fold greater, at least 60-fold greater, at least 65-fold greater, at least 70-fold greater, at least 75-fold greater, at least 80-fold greater, at least 85-fold greater, at least 90-fold greater, at least 95-fold greater, or at least 100-fold greater, or about 0.2-fold to about 100-fold greater, about 0.2-fold to about 90-fold greater, about 0.2-fold to about 80-fold greater, about 0.2-fold to about 70-fold greater, about 0.2-fold to about 60-fold greater, about 0.2-fold to about 50-fold greater, about 0.2-fold to about 40-fold greater, about 0.2-fold to about 30-fold greater, about 0.2-fold to about 25-fold greater, about 0.2-fold to about 20-fold greater, about 0.2-fold to about 15-fold greater, about 0.2-fold to about 10-fold greater, about 0.2-fold to about 8-fold greater, about 0.2-fold to about 5-fold greater, about 0.2-fold to about 2-fold greater, about 0.2-fold to about 1-fold greater, about 0.2-fold to about 0.5-fold greater, about 0.5-fold to about 100-fold greater, about 0.5-fold to about 90-fold greater, about 0.5-fold to about 80-fold greater, about 0.5-fold to about 70-fold greater, about 0.5-fold to about 60-fold greater, about 0.5-fold to about 50-fold greater, about 0.5-fold to about 40-fold greater, about 0.5-fold to about 30-fold greater, about 0.5-fold to about 25-fold greater, about 0.5-fold to about 20-fold greater, about 0.5-fold to about 15-fold greater, about 0.5-fold to about 10-fold greater, about 0.5-fold to about 8-fold greater, about 0.5-fold to about 5-fold greater, about 0.5-fold to about 2-fold greater, about 0.5-fold to about 1-fold greater, about 1-fold to about 100-fold greater, about 1-fold to about 90-fold greater, about 1-fold to about 80-fold greater, about 1-fold to about 70-fold greater, about 1-fold to about 60-fold greater, about 1-fold to about 50-fold greater, about 1-fold to about 40-fold greater, about 1-fold to about 30-fold greater, about 1-fold to about 25-fold greater, about 1-fold to about 20-fold greater, about 1-fold to about 15-fold greater, about 1-fold to about 10-fold greater, about 1-fold to about 8-fold greater, about 1-fold to about 5-fold greater, about 1-fold to about 2-fold greater, about 2-fold to about 100-fold greater, about 2-fold to about 90-fold greater, about 2-fold to about 80-fold greater, about 2-fold to about 70-fold greater, about 2-fold to about 60-fold greater, about 2-fold to about 50-fold greater, about 2-fold to about 40-fold greater, about 2-fold to about 30-fold greater, about 2-fold to about 25-fold greater, about 2-fold to about 20-fold greater, about 2-fold to about 15-fold greater, about 2-fold to about 10-fold greater, about 2-fold to about 8-fold greater, about 2-fold to about 5-fold greater, about 5-fold to about 100-fold greater, about 5-fold to about 90-fold greater, about 5-fold to about 80-fold greater, about 5-fold to about 70-fold greater, about 5-fold to about 60-fold greater, about 5-fold to about 50-fold greater, about 5-fold to about 40-fold greater, about 5-fold to about 30-fold greater, about 5-fold to about 25-fold greater, about 5-fold to about 20-fold greater, about 5-fold to about 15-fold greater, about 5-fold to about 10-fold greater, about 5-fold to about 8-fold greater, about 8-fold to about 100-fold greater, about 8-fold to about 90-fold greater, about 8-fold to about 80-fold greater, about 8-fold to about 70-fold greater, about 8-fold to about 60-fold greater, about 8-fold to about 50-fold greater, about 8-fold to about 40-fold greater, about 8-fold to about 30-fold greater, about 8-fold to about 25-fold greater, about 8-fold to about 20-fold greater, about 8-fold to about 15-fold greater, about 8-fold to about 10-fold greater, about 10-fold to about 100-fold greater, about 10-fold to about 90-fold greater, about 10-fold to about 80-fold greater, about 10-fold to about 70-fold greater, about 10-fold to about 60-fold greater, about 10-fold to about 50-fold greater, about 10-fold to about 40-fold greater, about 10-fold to about 30-fold greater, about 10-fold to about 25-fold greater, about 10-fold to about 20-fold greater, about 10-fold to about 15-fold greater, about 15-fold to about 100-fold greater, about 15-fold to about 90-fold greater, about 15-fold to about 80-fold greater, about 15-fold to about 70-fold greater, about 15-fold to about 60-fold greater, about 15-fold to about 50-fold greater, about 15-fold to about 40-fold greater, about 15-fold to about 30-fold greater, about 15-fold to about 25-fold greater, about 15-fold to about 20-fold greater, about 20-fold to about 100-fold greater, about 20-fold to about 90-fold greater, about 20-fold to about 80-fold greater, about 20-fold to about 70-fold greater, about 20-fold to about 60-fold greater, about 20-fold to about 50-fold greater, about 20-fold to about 40-fold greater, about 20-fold to about 30-fold greater, about 20-fold to about 25-fold greater, about 25-fold to about 100-fold greater, about 25-fold to about 90-fold greater, about 25-fold to about 80-fold greater, about 25-fold to about 70-fold greater, about 25-fold to about 60-fold greater, about 25-fold to about 50-fold greater, about 25-fold to about 40-fold greater, about 25-fold to about 30-fold greater, about 30-fold to about 100-fold greater, about 30-fold to about 90-fold greater, about 30-fold to about 80-fold greater, about 30-fold to about 70-fold greater, about 30-fold to about 60-fold greater, about 30-fold to about 50-fold greater, about 30-fold to about 40-fold greater, about 40-fold to about 100-fold greater, about 40-fold to about 90-fold greater, about 40-fold to about 80-fold greater, about 40-fold to about 70-fold greater, about 40-fold to about 60-fold greater, about 40-fold to about 50-fold greater, about 50-fold to about 100-fold greater, about 50-fold to about 90-fold greater, about 50-fold to about 80-fold greater, about 50-fold to about 70-fold greater, about 50-fold to about 60-fold greater, about 60-fold to about 100-fold greater, about 60-fold to about 90-fold greater, about 60-fold to about 80-fold greater, about 60-fold to about 70-fold greater, about 70-fold to about 100-fold greater, about 70-fold to about 90-fold greater, about 70-fold to about 80-fold greater, about 80-fold to about 100-fold greater, about 80-fold to about 90-fold greater, or about 90-fold to about 100-fold greater), than the K_{D} at a pH of about 7.0 to about 8.0 (e.g., any of the subranges of this range described herein).

In some embodiments of the ABPCs that include a first antigen-binding domain and a second antigen-binding domain, the first and second antigen-binding domains are identical or are at least 80% identical (e.g., at least 82%, at least 84%, at least 86%, at least 88%, at least 90%, at least 91%, at least 92%, at least 93%, at least 94%, at least 95%, at least 96%, at least 97%, at least 98%, or at least 99% identical) in amino acid sequence to each other. In some embodiments, the ABPCs that include a first antigen-binding domain and a second antigen-binding domain, the first antigen-binding domain and the second antigen-binding domain have a sequence that is less than 80% identical (e.g., less than 75% identical, less than 70% identical, less than 65% identical, less than 60% identical, less than 55% identical, less than 50% identical, less than 45% identical, less than 40% identical, less than 35% identical, less than 30% identical, less than 25% identical, less than 20% identical, less than 15% identical, less than 10% identical, or less than 5% identical) to each other. In some embodiments of ABPCs that include a first and a second antigen-binding domain, the first and second antigen-binding domain binds two different epitopes (e.g., two different epitopes on DLL3 or the first antigen-binding domain binding specifically to DLL3 and the second antigen-binding domain binding to an antigen other than DLL3).

In some embodiments of any of the ABPCs described herein, the K_{D} of the first antigen-binding domain (and optionally, the second antigen-binding domain if present) at a pH of about 7.0 to about 8.0 (e.g., any of the subranges of this range described herein) is between about 1 pM to about 5 μM (e.g., about 1 pM to about 2 μM, about 1 pM to about 1 μM, about 1 pM to about 500 nM, about 1 pM to about 250 nM, about 1 pM to about 240 nM, about 1 pM to about 230 nM, about 1 pM to about 220 nM, about 1 pM to about 210 nM, about 1 pM to about 200 nM, about 1 pM to about 190 nM, about 1 pM to about 180 nM, about 1 pM to about 170 nM, about 1 pM to about 160 nM, about 1 pM to about 150 nM, about 1 pM to about 140 nM, about 1 pM to about 130 nM, about 1 pM to about 120 nM, about 1 pM to about 110 nM, about 1 pM to about 100 nM, about 1 pM to about 95 nM, about 1 pM to about 90 nM, about 1 pM to about 85 nM, about 1 pM to about 80 nM, about 1 pM to about 75 nM, about 1 pM to about 70 nM, about 1 pM to about 65 nM, about 1 pM to about 60 nM, about 1 pM to about 55 nM, about 1 pM to about 50 nM, about 1 pM to about 45 nM, about 1 pM to about 40 nM, about 1 pM to about 35 nM, about 1 pM to about 30 nM, about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 μM, about 2 pM to about 5 μM, about 2 pM to about 2 μM, about 2 pM to about 1 μM, about 2 pM to about 500 nM, about 2 pM to about 250 nM, about 2 pM to about 240 nM, about 2 pM to about 230 nM, about 2 pM to about 220 nM, about 2 pM to about 210 nM, about 2 pM to about 200 nM, about 2 pM to about 190 nM, about 2 pM to about 180 nM, about 2 pM to about 170 nM, about 2 pM to about 160 nM, about 2 pM to about 150 nM, about 2 pM to about 140 nM, about 2 pM to about 130 nM, about 2 pM to about 120 nM, about 2 pM to about 110 nM, about 2 pM to about 100 nM, about 2 pM to about 95 nM, about 2 pM to about 90 nM, about 2 pM to about 85 nM, about 2 pM to about 80 nM, about 2 pM to about 75 nM, about 2 pM to about 70 nM, about 2 pM to about 65 nM, about 2 pM to about 60 nM, about 2 pM to about 55 nM, about 2 pM to about 50 nM, about 2 pM to about 45 nM, about 2 pM to about 40 nM, about 2 pM to about 35 nM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 5 μM, about 5 pM to about 2 μM, about 5 pM to about 1 μM, about 5 pM to about 500 nM, about 5 pM to about 250 nM, about 5 pM to about 240 nM, about 5 pM to about 230 nM, about 5 pM to about 220 nM, about 5 pM to about 210 nM, about 5 pM to about 200 nM, about 5 pM to about 190 nM, about 5 pM to about 180 nM, about 5 pM to about 170 nM, about 5 pM to about 160 nM, about 5 pM to about 150 nM, about 5 pM to about 140 nM, about 5 pM to about 130 nM, about 5 pM to about 120 nM, about 5 pM to about 110 nM, about 5 pM to about 100 nM, about 5 pM to about 95 nM, about 5 pM to about 90 nM, about 5 pM to about 85 nM, about 5 pM to about 80 nM, about 5 pM to about 75 nM, about 5 pM to about 70 nM, about 5 pM to about 65 nM, about 5 pM to about 60 nM, about 5 pM to about 55 nM, about 5 pM to about 50 nM, about 5 pM to about 45 nM, about 5 pM to about 40 nM, about 5 pM to about 35 nM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 5 pM, about 10 pM to about 2 pM, about 10 pM to about 1 μM, about 10 pM to about 500 nM, about 10 pM to about 250 nM, about 10 pM to about 240 nM, about 10 pM to about 230 nM, about 10 pM to about 220 nM, about 10 pM to about 210 nM, about 10 pM to about 200 nM, about 10 pM to about 190 nM, about 10 pM to about 180 nM, about 10 pM to about 170 nM, about 10 pM to about 160 nM, about 10 pM to about 150 nM, about 10 pM to about 140 nM, about 10 pM to about 130 nM, about 10 pM to about 120 nM, about 10 pM to about 110 nM, about 10 pM to about 100 nM, about 10 pM to about 95 nM, about 10 pM to about 90 nM, about 10 pM to about 85 nM, about 10 pM to about 80 nM, about 10 pM to about 75 nM, about 10 pM to about 70 nM, about 10 pM to about 65 nM, about 10 pM to about 60 nM, about 10 pM to about 55 nM, about 10 pM to about 50 nM, about 10 pM to about 45 nM, about 10 pM to about 40 nM, about 10 pM to about 35 nM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 5 μM, about 15 pM to about 2 μM, about 15 pM to about 1 μM, about 15 pM to about 500 nM, about 15 pM to about 250 nM, about 15 pM to about 240 nM, about 15 pM to about 230 nM, about 15 pM to about 220 nM, about 15 pM to about 210 nM, about 15 pM to about 200 nM, about 15 pM to about 190 nM, about 15 pM to about 180 nM, about 15 pM to about 170 nM, about 15 pM to about 160 nM, about 15 pM to about 150 nM, about 15 pM to about 140 nM, about 15 pM to about 130 nM, about 15 pM to about 120 nM, about 15 pM to about 110 nM, about 15 pM to about 100 nM, about 15 pM to about 95 nM, about 15 pM to about 90 nM, about 15 pM to about 85 nM, about 15 pM to about 80 nM, about 15 pM to about 75 nM, about 15 pM to about 70 nM, about 15 pM to about 65 nM, about 15 pM to about 60 nM, about 15 pM to about 55 nM, about 15 pM to about 50 nM, about 15 pM to about 45 nM, about 15 pM to about 40 nM, about 15 pM to about 35 nM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 5 pM, about 20 pM to about 2 μM, about 20 pM to about 1 μM, about 20 pM to about 500 nM, about 20 pM to about 250 nM, about 20 pM to about 240 nM, about 20 pM to about 230 nM, about 20 pM to about 220 nM, about 20 pM to about 210 nM, about 20 pM to about 200 nM, about 20 pM to about 190 nM, about 20 pM to about 180 nM, about 20 pM to about 170 nM, about 20 pM to about 160 nM, about 20 pM to about 150 nM, about 20 pM to about 140 nM, about 20 pM to about 130 nM, about 20 pM to about 120 nM, about 20 pM to about 110 nM, about 20 pM to about 100 nM, about 20 pM to about 95 nM, about 20 pM to about 90 nM, about 20 pM to about 85 nM, about 20 pM to about 80 nM, about 20 pM to about 75 nM, about 20 pM to about 70 nM, about 20 pM to about 65 nM, about 20 pM to about 60 nM, about 20 pM to about 55 nM, about 20 pM to about 50 nM, about 20 pM to about 45 nM, about 20 pM to about 40 nM, about 20 pM to about 35 nM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 5 μM, about 30 pM to about 2 μM, about 30 pM to about 1 μM, about 30 pM to about 500 nM, about 30 pM to about 250 nM, about 30 pM to about 240 nM, about 30 pM to about 230 nM, about 30 pM to about 220 nM, about 30 pM to about 210 nM, about 30 pM to about 200 nM, about 30 pM to about 190 nM, about 30 pM to about 180 nM, about 30 pM to about 170 nM, about 30 pM to about 160 nM, about 30 pM to about 150 nM, about 30 pM to about 140 nM, about 30 pM to about 130 nM, about 30 pM to about 120 nM, about 30 pM to about 110 nM, about 30 pM to about 100 nM, about 30 pM to about 95 nM, about 30 pM to about 90 nM, about 30 pM to about 85 nM, about 30 pM to about 80 nM, about 30 pM to about 75 nM, about 30 pM to about 70 nM, about 30 pM to about 65 nM, about 30 pM to about 60 nM, about 30 pM to about 55 nM, about 30 pM to about 50 nM, about 30 pM to about 45 nM, about 30 pM to about 40 nM, about 30 pM to about 35 nM, about 30 pM to about 30 nM, about 30 pM to about 25 nM, about 30 pM to about 20 nM, about 30 pM to about 15 nM, about 30 pM to about 10 nM, about 30 pM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 pM to about 650 pM, about 30 pM to about 600 pM, about 30 pM to about 550 pM, about 30 pM to about 500 pM, about 30 pM to about 450 pM, about 30 pM to about 400 pM, about 30 pM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 5 μM, about 40 pM to about 2 μM, about 40 pM to about 1 μM, about 40 pM to about 500 nM, about 40 pM to about 250 nM, about 40 pM to about 240 nM, about 40 pM to about 230 nM, about 40 pM to about 220 nM, about 40 pM to about 210 nM, about 40 pM to about 200 nM, about 40 pM to about 190 nM, about 40 pM to about 180 nM, about 40 pM to about 170 nM, about 40 pM to about 160 nM, about 40 pM to about 150 nM, about 40 pM to about 140 nM, about 40 pM to about 130 nM, about 40 pM to about 120 nM, about 40 pM to about 110 nM, about 40 pM to about 100 nM, about 40 pM to about 95 nM, about 40 pM to about 90 nM, about 40 pM to about 85 nM, about 40 pM to about 80 nM, about 40 pM to about 75 nM, about 40 pM to about 70 nM, about 40 pM to about 65 nM, about 40 pM to about 60 nM, about 40 pM to about 55 nM, about 40 pM to about 50 nM, about 40 pM to about 45 nM, about 40 pM to about 40 nM, about 40 pM to about 35 nM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 5 μM, about 50 pM to about 2 μM, about 50 pM to about 1 μM, about 50 pM to about 500 nM, about 50 pM to about 250 nM, about 50 pM to about 240 nM, about 50 pM to about 230 nM, about 50 pM to about 220 nM, about 50 pM to about 210 nM, about 50 pM to about 200 nM, about 50 pM to about 190 nM, about 50 pM to about 180 nM, about 50 pM to about 170 nM, about 50 pM to about 160 nM, about 50 pM to about 150 nM, about 50 pM to about 140 nM, about 50 pM to about 130 nM, about 50 pM to about 120 nM, about 50 pM to about 110 nM, about 50 pM to about 100 nM, about 50 pM to about 95 nM, about 50 pM to about 90 nM, about 50 pM to about 85 nM, about 50 pM to about 80 nM, about 50 pM to about 75 nM, about 50 pM to about 70 nM, about 50 pM to about 65 nM, about 50 pM to about 60 nM, about 50 pM to about 55 nM, about 50 pM to about 50 nM, about 50 pM to about 45 nM, about 50 pM to about 40 nM, about 50 pM to about 35 nM, about 50 pM to about 30 nM, about 50 pM to about 25 nM, about 50 pM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 5 μM, about 60 pM to about 2 μM, about 60 pM to about 1 μM, about 60 pM to about 500 nM, about 60 pM to about 250 nM, about 60 pM to about 240 nM, about 60 pM to about 230 nM, about 60 pM to about 220 nM, about 60 pM to about 210 nM, about 60 pM to about 200 nM, about 60 pM to about 190 nM, about 60 pM to about 180 nM, about 60 pM to about 170 nM, about 60 pM to about 160 nM, about 60 pM to about 150 nM, about 60 pM to about 140 nM, about 60 pM to about 130 nM, about 60 pM to about 120 nM, about 60 pM to about 110 nM, about 60 pM to about 100 nM, about 60 pM to about 95 nM, about 60 pM to about 90 nM, about 60 pM to about 85 nM, about 60 pM to about 80 nM, about 60 pM to about 75 nM, about 60 pM to about 70 nM, about 60 pM to about 65 nM, about 60 pM to about 60 nM, about 60 pM to about 55 nM, about 60 pM to about 50 nM, about 60 pM to about 45 nM, about 60 pM to about 40 nM, about 60 pM to about 35 nM, about 60 pM to about 30 nM, about 60 pM to about 25 nM, about 60 pM to about 20 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 5 μM, about 70 pM to about 2 μM, about 70 pM to about 1 μM, about 70 pM to about 500 nM, about 70 pM to about 250 nM, about 70 pM to about 240 nM, about 70 pM to about 230 nM, about 70 pM to about 220 nM, about 70 pM to about 210 nM, about 70 pM to about 200 nM, about 70 pM to about 190 nM, about 70 pM to about 180 nM, about 70 pM to about 170 nM, about 70 pM to about 160 nM, about 70 pM to about 150 nM, about 70 pM to about 140 nM, about 70 pM to about 130 nM, about 70 pM to about 120 nM, about 70 pM to about 110 nM, about 70 pM to about 100 nM, about 70 pM to about 95 nM, about 70 pM to about 90 nM, about 70 pM to about 85 nM, about 70 pM to about 80 nM, about 70 pM to about 75 nM, about 70 pM to about 70 nM, about 70 pM to about 65 nM, about 70 pM to about 60 nM, about 70 pM to about 55 nM, about 70 pM to about 50 nM, about 70 pM to about 45 nM, about 70 pM to about 40 nM, about 70 pM to about 35 nM, about 70 pM to about 30 nM, about 70 pM to about 25 nM, about 70 pM to about 20 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 5 μM, about 80 pM to about 2 μM, about 80 pM to about 1 μM, about 80 pM to about 500 nM, about 80 pM to about 250 nM, about 80 pM to about 240 nM, about 80 pM to about 230 nM, about 80 pM to about 220 nM, about 80 pM to about 210 nM, about 80 pM to about 200 nM, about 80 pM to about 190 nM, about 80 pM to about 180 nM, about 80 pM to about 170 nM, about 80 pM to about 160 nM, about 80 pM to about 150 nM, about 80 pM to about 140 nM, about 80 pM to about 130 nM, about 80 pM to about 120 nM, about 80 pM to about 110 nM, about 80 pM to about 100 nM, about 80 pM to about 95 nM, about 80 pM to about 90 nM, about 80 pM to about 85 nM, about 80 pM to about 80 nM, about 80 pM to about 75 nM, about 80 pM to about 70 nM, about 80 pM to about 65 nM, about 80 pM to about 60 nM, about 80 pM to about 55 nM, about 80 pM to about 50 nM, about 80 pM to about 45 nM, about 80 pM to about 40 nM, about 80 pM to about 35 nM, about 80 pM to about 30 nM, about 80 pM to about 25 nM, about 80 pM to about 20 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 5 μM, about 90 pM to about 2 μM, about 90 pM to about 1 μM, about 90 pM to about 500 nM, about 90 pM to about 250 nM, about 90 pM to about 240 nM, about 90 pM to about 230 nM, about 90 pM to about 220 nM, about 90 pM to about 210 nM, about 90 pM to about 200 nM, about 90 pM to about 190 nM, about 90 pM to about 180 nM, about 90 pM to about 170 nM, about 90 pM to about 160 nM, about 90 pM to about 150 nM, about 90 pM to about 140 nM, about 90 pM to about 130 nM, about 90 pM to about 120 nM, about 90 pM to about 110 nM, about 90 pM to about 100 nM, about 90 pM to about 95 nM, about 90 pM to about 90 nM, about 90 pM to about 85 nM, about 90 pM to about 80 nM, about 90 pM to about 75 nM, about 90 pM to about 70 nM, about 90 pM to about 65 nM, about 90 pM to about 60 nM, about 90 pM to about 55 nM, about 90 pM to about 50 nM, about 90 pM to about 45 nM, about 90 pM to about 40 nM, about 90 pM to about 35 nM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 5 μM, about 100 pM to about 2 μM, about 100 pM to about 1 μM, about 100 pM to about 500 nM, about 100 pM to about 250 nM, about 100 pM to about 240 nM, about 100 pM to about 230 nM, about 100 pM to about 220 nM, about 100 pM to about 210 nM, about 100 pM to about 200 nM, about 100 pM to about 190 nM, about 100 pM to about 180 nM, about 100 pM to about 170 nM, about 100 pM to about 160 nM, about 100 pM to about 150 nM, about 100 pM to about 140 nM, about 100 pM to about 130 nM, about 100 pM to about 120 nM, about 100 pM to about 110 nM, about 100 pM to about 100 nM, about 100 pM to about 95 nM, about 100 pM to about 90 nM, about 100 pM to about 85 nM, about 100 pM to about 80 nM, about 100 pM to about 75 nM, about 100 pM to about 70 nM, about 100 pM to about 65 nM, about 100 pM to about 60 nM, about 100 pM to about 55 nM, about 100 pM to about 50 nM, about 100 pM to about 45 nM, about 100 pM to about 40 nM, about 100 pM to about 35 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 5 μM, about 150 pM to about 2 μM, about 150 pM to about 1 μM, about 150 pM to about 500 nM, about 150 pM to about 250 nM, about 150 pM to about 240 nM, about 150 pM to about 230 nM, about 150 pM to about 220 nM, about 150 pM to about 210 nM, about 150 pM to about 200 nM, about 150 pM to about 190 nM, about 150 pM to about 180 nM, about 150 pM to about 170 nM, about 150 pM to about 160 nM, about 150 pM to about 150 nM, about 150 pM to about 140 nM, about 150 pM to about 130 nM, about 150 pM to about 120 nM, about 150 pM to about 110 nM, about 150 pM to about 100 nM, about 150 pM to about 95 nM, about 150 pM to about 90 nM, about 150 pM to about 85 nM, about 150 pM to about 80 nM, about 150 pM to about 75 nM, about 150 pM to about 70 nM, about 150 pM to about 65 nM, about 150 pM to about 60 nM, about 150 pM to about 55 nM, about 150 pM to about 50 nM, about 150 pM to about 45 nM, about 150 pM to about 40 nM, about 150 pM to about 35 nM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 5 μM, about 200 pM to about 2 μM, about 200 pM to about 1 μM, about 200 pM to about 500 nM, about 200 pM to about 250 nM, about 200 pM to about 240 nM, about 200 pM to about 230 nM, about 200 pM to about 220 nM, about 200 pM to about 210 nM, about 200 pM to about 200 nM, about 200 pM to about 190 nM, about 200 pM to about 180 nM, about 200 pM to about 170 nM, about 200 pM to about 160 nM, about 200 pM to about 150 nM, about 200 pM to about 140 nM, about 200 pM to about 130 nM, about 200 pM to about 120 nM, about 200 pM to about 110 nM, about 200 pM to about 100 nM, about 200 pM to about 95 nM, about 200 pM to about 90 nM, about 200 pM to about 85 nM, about 200 pM to about 80 nM, about 200 pM to about 75 nM, about 200 pM to about 70 nM, about 200 pM to about 65 nM, about 200 pM to about 60 nM, about 200 pM to about 55 nM, about 200 pM to about 50 nM, about 200 pM to about 45 nM, about 200 pM to about 40 nM, about 200 pM to about 35 nM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 5 μM, about 300 pM to about 2 μM, about 300 pM to about 1 μM, about 300 pM to about 500 nM, about 300 pM to about 250 nM, about 300 pM to about 240 nM, about 300 pM to about 230 nM, about 300 pM to about 220 nM, about 300 pM to about 210 nM, about 300 pM to about 200 nM, about 300 pM to about 190 nM, about 300 pM to about 180 nM, about 300 pM to about 170 nM, about 300 pM to about 160 nM, about 300 pM to about 150 nM, about 300 pM to about 140 nM, about 300 pM to about 130 nM, about 300 pM to about 120 nM, about 300 pM to about 110 nM, about 300 pM to about 100 nM, about 300 pM to about 95 nM, about 300 pM to about 90 nM, about 300 pM to about 85 nM, about 300 pM to about 80 nM, about 300 pM to about 75 nM, about 300 pM to about 70 nM, about 300 pM to about 65 nM, about 300 pM to about 60 nM, about 300 pM to about 55 nM, about 300 pM to about 50 nM, about 300 pM to about 45 nM, about 300 pM to about 40 nM, about 300 pM to about 35 nM, about 300 pM to about 30 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 5 μM, about 400 pM to about 2 μM, about 400 pM to about 1 μM, about 400 pM to about 500 nM, about 400 pM to about 250 nM, about 400 pM to about 240 nM, about 400 pM to about 230 nM, about 400 pM to about 220 nM, about 400 pM to about 210 nM, about 400 pM to about 200 nM, about 400 pM to about 190 nM, about 400 pM to about 180 nM, about 400 pM to about 170 nM, about 400 pM to about 160 nM, about 400 pM to about 150 nM, about 400 pM to about 140 nM, about 400 pM to about 130 nM, about 400 pM to about 120 nM, about 400 pM to about 110 nM, about 400 pM to about 100 nM, about 400 pM to about 95 nM, about 400 pM to about 90 nM, about 400 pM to about 85 nM, about 400 pM to about 80 nM, about 400 pM to about 75 nM, about 400 pM to about 70 nM, about 400 pM to about 65 nM, about 400 pM to about 60 nM, about 400 pM to about 55 nM, about 400 pM to about 50 nM, about 400 pM to about 45 nM, about 400 pM to about 40 nM, about 400 pM to about 35 nM, about 400 pM to about 30 nM, about 400 pM to about 25 nM, about 400 pM to about 20 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 5 μM, about 500 pM to about 2 μM, about 500 pM to about 1 μM, about 500 pM to about 500 nM, about 500 pM to about 250 nM, about 500 pM to about 240 nM, about 500 pM to about 230 nM, about 500 pM to about 220 nM, about 500 pM to about 210 nM, about 500 pM to about 200 nM, about 500 pM to about 190 nM, about 500 pM to about 180 nM, about 500 pM to about 170 nM, about 500 pM to about 160 nM, about 500 pM to about 150 nM, about 500 pM to about 140 nM, about 500 pM to about 130 nM, about 500 pM to about 120 nM, about 500 pM to about 110 nM, about 500 pM to about 100 nM, about 500 pM to about 95 nM, about 500 pM to about 90 nM, about 500 pM to about 85 nM, about 500 pM to about 80 nM, about 500 pM to about 75 nM, about 500 pM to about 70 nM, about 500 pM to about 65 nM, about 500 pM to about 60 nM, about 500 pM to about 55 nM, about 500 pM to about 50 nM, about 500 pM to about 45 nM, about 500 pM to about 40 nM, about 500 pM to about 35 nM, about 500 pM to about 30 nM, about 500 pM to about 25 nM, about 500 pM to about 20 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 5 μM, about 600 pM to about 2 μM, about 600 pM to about 1 μM, about 600 pM to about 500 nM, about 600 pM to about 250 nM, about 600 pM to about 240 nM, about 600 pM to about 230 nM, about 600 pM to about 220 nM, about 600 pM to about 210 nM, about 600 pM to about 200 nM, about 600 pM to about 190 nM, about 600 pM to about 180 nM, about 600 pM to about 170 nM, about 600 pM to about 160 nM, about 600 pM to about 150 nM, about 600 pM to about 140 nM, about 600 pM to about 130 nM, about 600 pM to about 120 nM, about 600 pM to about 110 nM, about 600 pM to about 100 nM, about 600 pM to about 95 nM, about 600 pM to about 90 nM, about 600 pM to about 85 nM, about 600 pM to about 80 nM, about 600 pM to about 75 nM, about 600 pM to about 70 nM, about 600 pM to about 65 nM, about 600 pM to about 60 nM, about 600 pM to about 55 nM, about 600 pM to about 50 nM, about 600 pM to about 45 nM, about 600 pM to about 40 nM, about 600 pM to about 35 nM, about 600 pM to about 30 nM, about 600 pM to about 25 nM, about 600 pM to about 20 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 5 μM, about 700 pM to about 2 μM, about 700 pM to about 1 μM, about 700 pM to about 500 nM, about 700 pM to about 250 nM, about 700 pM to about 240 nM, about 700 pM to about 230 nM, about 700 pM to about 220 nM, about 700 pM to about 210 nM, about 700 pM to about 200 nM, about 700 pM to about 190 nM, about 700 pM to about 180 nM, about 700 pM to about 170 nM, about 700 pM to about 160 nM, about 700 pM to about 150 nM, about 700 pM to about 140 nM, about 700 pM to about 130 nM, about 700 pM to about 120 nM, about 700 pM to about 110 nM, about 700 pM to about 100 nM, about 700 pM to about 95 nM, about 700 pM to about 90 nM, about 700 pM to about 85 nM, about 700 pM to about 80 nM, about 700 pM to about 75 nM, about 700 pM to about 70 nM, about 700 pM to about 65 nM, about 700 pM to about 60 nM, about 700 pM to about 55 nM, about 700 pM to about 50 nM, about 700 pM to about 45 nM, about 700 pM to about 40 nM, about 700 pM to about 35 nM, about 700 pM to about 30 nM, about 700 pM to about 25 nM, about 700 pM to about 20 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 5 μM, about 800 pM to about 2 μM, about 800 pM to about 1 μM, about 800 pM to about 500 nM, about 800 pM to about 250 nM, about 800 pM to about 240 nM, about 800 pM to about 230 nM, about 800 pM to about 220 nM, about 800 pM to about 210 nM, about 800 pM to about 200 nM, about 800 pM to about 190 nM, about 800 pM to about 180 nM, about 800 pM to about 170 nM, about 800 pM to about 160 nM, about 800 pM to about 150 nM, about 800 pM to about 140 nM, about 800 pM to about 130 nM, about 800 pM to about 120 nM, about 800 pM to about 110 nM, about 800 pM to about 100 nM, about 800 pM to about 95 nM, about 800 pM to about 90 nM, about 800 pM to about 85 nM, about 800 pM to about 80 nM, about 800 pM to about 75 nM, about 800 pM to about 70 nM, about 800 pM to about 65 nM, about 800 pM to about 60 nM, about 800 pM to about 55 nM, about 800 pM to about 50 nM, about 800 pM to about 45 nM, about 800 pM to about 40 nM, about 800 pM to about 35 nM, about 800 pM to about 30 nM, about 800 pM to about 25 nM, about 800 pM to about 20 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 5 μM, about 900 pM to about 2 μM, about 900 pM to about 1 μM, about 900 pM to about 500 nM, about 900 pM to about 250 nM, about 900 pM to about 240 nM, about 900 pM to about 230 nM, about 900 pM to about 220 nM, about 900 pM to about 210 nM, about 900 pM to about 200 nM, about 900 pM to about 190 nM, about 900 pM to about 180 nM, about 900 pM to about 170 nM, about 900 pM to about 160 nM, about 900 pM to about 150 nM, about 900 pM to about 140 nM, about 900 pM to about 130 nM, about 900 pM to about 120 nM, about 900 pM to about 110 nM, about 900 pM to about 100 nM, about 900 pM to about 95 nM, about 900 pM to about 90 nM, about 900 pM to about 85 nM, about 900 pM to about 80 nM, about 900 pM to about 75 nM, about 900 pM to about 70 nM, about 900 pM to about 65 nM, about 900 pM to about 60 nM, about 900 pM to about 55 nM, about 900 pM to about 50 nM, about 900 pM to about 45 nM, about 900 pM to about 40 nM, about 900 pM to about 35 nM, about 900 pM to about 30 nM, about 900 pM to about 25 nM, about 900 pM to about 20 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 5 μM, about 1 nM to about 2 μM, about 1 nM to about 1 μM, about 1 nM to about 500 nM, about 1 nM to about 250 nM, about 1 nM to about 240 nM, about 1 nM to about 230 nM, about 1 nM to about 220 nM, about 1 nM to about 210 nM, about 1 nM to about 200 nM, about 1 nM to about 190 nM, about 1 nM to about 180 nM, about 1 nM to about 170 nM, about 1 nM to about 160 nM, about 1 nM to about 150 nM, about 1 nM to about 140 nM, about 1 nM to about 130 nM, about 1 nM to about 120 nM, about 1 nM to about 110 nM, about 1 nM to about 100 nM, about 1 nM to about 95 nM, about 1 nM to about 90 nM, about 1 nM to about 85 nM, about 1 nM to about 80 nM, about 1 nM to about 75 nM, about 1 nM to about 70 nM, about 1 nM to about 65 nM, about 1 nM to about 60 nM, about 1 nM to about 55 nM, about 1 nM to about 50 nM, about 1 nM to about 45 nM, about 1 nM to about 40 nM, about 1 nM to about 35 nM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 5 μM, about 2 nM to about 2 μM, about 2 nM to about 1 μM, about 2 nM to about 500 nM, about 2 nM to about 250 nM, about 2 nM to about 240 nM, about 2 nM to about 230 nM, about 2 nM to about 220 nM, about 2 nM to about 210 nM, about 2 nM to about 200 nM, about 2 nM to about 190 nM, about 2 nM to about 180 nM, about 2 nM to about 170 nM, about 2 nM to about 160 nM, about 2 nM to about 150 nM, about 2 nM to about 140 nM, about 2 nM to about 130 nM, about 2 nM to about 120 nM, about 2 nM to about 110 nM, about 2 nM to about 100 nM, about 2 nM to about 95 nM, about 2 nM to about 90 nM, about 2 nM to about 85 nM, about 2 nM to about 80 nM, about 2 nM to about 75 nM, about 2 nM to about 70 nM, about 2 nM to about 65 nM, about 2 nM to about 60 nM, about 2 nM to about 55 nM, about 2 nM to about 50 nM, about 2 nM to about 45 nM, about 2 nM to about 40 nM, about 2 nM to about 35 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 5 μM, about 4 nM to about 2 μM, about 4 nM to about 1 μM, about 4 nM to about 500 nM, about 4 nM to about 250 nM, about 4 nM to about 240 nM, about 4 nM to about 230 nM, about 4 nM to about 220 nM, about 4 nM to about 210 nM, about 4 nM to about 200 nM, about 4 nM to about 190 nM, about 4 nM to about 180 nM, about 4 nM to about 170 nM, about 4 nM to about 160 nM, about 4 nM to about 150 nM, about 4 nM to about 140 nM, about 4 nM to about 130 nM, about 4 nM to about 120 nM, about 4 nM to about 110 nM, about 4 nM to about 100 nM, about 4 nM to about 95 nM, about 4 nM to about 90 nM, about 4 nM to about 85 nM, about 4 nM to about 80 nM, about 4 nM to about 75 nM, about 4 nM to about 70 nM, about 4 nM to about 65 nM, about 4 nM to about 60 nM, about 4 nM to about 55 nM, about 4 nM to about 50 nM, about 4 nM to about 45 nM, about 4 nM to about 40 nM, about 4 nM to about 35 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 5 μM, about 5 nM to about 2 μM, about 5 nM to about 1 μM, about 5 nM to about 500 nM, about 5 nM to about 250 nM, about 5 nM to about 240 nM, about 5 nM to about 230 nM, about 5 nM to about 220 nM, about 5 nM to about 210 nM, about 5 nM to about 200 nM, about 5 nM to about 190 nM, about 5 nM to about 180 nM, about 5 nM to about 170 nM, about 5 nM to about 160 nM, about 5 nM to about 150 nM, about 5 nM to about 140 nM, about 5 nM to about 130 nM, about 5 nM to about 120 nM, about 5 nM to about 110 nM, about 5 nM to about 100 nM, about 5 nM to about 95 nM, about 5 nM to about 90 nM, about 5 nM to about 85 nM, about 5 nM to about 80 nM, about 5 nM to about 75 nM, about 5 nM to about 70 nM, about 5 nM to about 65 nM, about 5 nM to about 60 nM, about 5 nM to about 55 nM, about 5 nM to about 50 nM, about 5 nM to about 45 nM, about 5 nM to about 40 nM, about 5 nM to about 35 nM, about 5 nM to about 30 nM, about 5 nM to about 25 nM, about 5 nM to about 20 nM, about 5 nM to about 15 nM, about 5 nM to about 10 nM, about 10 nM to about 5 μM, about 10 nM to about 2 μM, about 10 nM to about 1 μM, about 10 nM to about 500 nM, about 10 nM to about 250 nM, about 10 nM to about 240 nM, about 10 nM to about 230 nM, about 10 nM to about 220 nM, about 10 nM to about 210 nM, about 10 nM to about 200 nM, about 10 nM to about 190 nM, about 10 nM to about 180 nM, about 10 nM to about 170 nM, about 10 nM to about 160 nM, about 10 nM to about 150 nM, about 10 nM to about 140 nM, about 10 nM to about 130 nM, about 10 nM to about 120 nM, about 10 nM to about 110 nM, about 10 nM to about 100 nM, about 10 nM to about 95 nM, about 10 nM to about 90 nM, about 10 nM to about 85 nM, about 10 nM to about 80 nM, about 10 nM to about 75 nM, about 10 nM to about 70 nM, about 10 nM to about 65 nM, about 10 nM to about 60 nM, about 10 nM to about 55 nM, about 10 nM to about 50 nM, about 10 nM to about 45 nM, about 10 nM to about 40 nM, about 10 nM to about 35 nM, about 10 nM to about 30 nM, about 10 nM to about 25 nM, about 10 nM to about 20 nM, about 10 nM to about 15 nM, about 15 nM to about 5 μM, about 15 nM to about 2 μM, about 15 nM to about 1 μM, about 15 nM to about 500 nM, about 15 nM to about 250 nM, about 15 nM to about 240 nM, about 15 nM to about 230 nM, about 15 nM to about 220 nM, about 15 nM to about 210 nM, about 15 nM to about 200 nM, about 15 nM to about 190 nM, about 15 nM to about 180 nM, about 15 nM to about 170 nM, about 15 nM to about 160 nM, about 15 nM to about 150 nM, about 15 nM to about 140 nM, about 15 nM to about 130 nM, about 15 nM to about 120 nM, about 15 nM to about 110 nM, about 15 nM to about 100 nM, about 15 nM to about 95 nM, about 15 nM to about 90 nM, about 15 nM to about 85 nM, about 15 nM to about 80 nM, about 15 nM to about 75 nM, about 15 nM to about 70 nM, about 15 nM to about 65 nM, about 15 nM to about 60 nM, about 15 nM to about 55 nM, about 15 nM to about 50 nM, about 15 nM to about 45 nM, about 15 nM to about 40 nM, about 15 nM to about 35 nM, about 15 nM to about 30 nM, about 15 nM to about 25 nM, about 15 nM to about 20 nM, about 20 nM to about 5 μM, about 20 nM to about 2 μM, about 20 nM to about 1 μM, about 20 nM to about 500 nM, about 20 nM to about 250 nM, about 20 nM to about 240 nM, about 20 nM to about 230 nM, about 20 nM to about 220 nM, about 20 nM to about 210 nM, about 20 nM to about 200 nM, about 20 nM to about 190 nM, about 20 nM to about 180 nM, about 20 nM to about 170 nM, about 20 nM to about 160 nM, about 20 nM to about 150 nM, about 20 nM to about 140 nM, about 20 nM to about 130 nM, about 20 nM to about 120 nM, about 20 nM to about 110 nM, about 20 nM to about 100 nM, about 20 nM to about 95 nM, about 20 nM to about 90 nM, about 20 nM to about 85 nM, about 20 nM to about 80 nM, about 20 nM to about 75 nM, about 20 nM to about 70 nM, about 20 nM to about 65 nM, about 20 nM to about 60 nM, about 20 nM to about 55 nM, about 20 nM to about 50 nM, about 20 nM to about 45 nM, about 20 nM to about 40 nM, about 20 nM to about 35 nM, about 20 nM to about 30 nM, about 20 nM to about 25 nM, about 25 nM to about 5 μM, about 25 nM to about 2 μM, about 25 nM to about 1 μM, about 25 nM to about 500 nM, about 25 nM to about 250 nM, about 25 nM to about 240 nM, about 25 nM to about 230 nM, about 25 nM to about 220 nM, about 25 nM to about 210 nM, about 25 nM to about 200 nM, about 25 nM to about 190 nM, about 25 nM to about 180 nM, about 25 nM to about 170 nM, about 25 nM to about 160 nM, about 25 nM to about 150 nM, about 25 nM to about 140 nM, about 25 nM to about 130 nM, about 25 nM to about 120 nM, about 25 nM to about 110 nM, about 25 nM to about 100 nM, about 25 nM to about 95 nM, about 25 nM to about 90 nM, about 25 nM to about 85 nM, about 25 nM to about 80 nM, about 25 nM to about 75 nM, about 25 nM to about 70 nM, about 25 nM to about 65 nM, about 25 nM to about 60 nM, about 25 nM to about 55 nM, about 25 nM to about 50 nM, about 25 nM to about 45 nM, about 25 nM to about 40 nM, about 25 nM to about 35 nM, about 25 nM to about 30 nM, about 30 nM to about 5 μM, about 30 nM to about 2 μM, about 30 nM to about 1 μM, about 30 nM to about 500 nM, about 30 nM to about 250 nM, about 30 nM to about 240 nM, about 30 nM to about 230 nM, about 30 nM to about 220 nM, about 30 nM to about 210 nM, about 30 nM to about 200 nM, about 30 nM to about 190 nM, about 30 nM to about 180 nM, about 30 nM to about 170 nM, about 30 nM to about 160 nM, about 30 nM to about 150 nM, about 30 nM to about 140 nM, about 30 nM to about 130 nM, about 30 nM to about 120 nM, about 30 nM to about 110 nM, about 30 nM to about 100 nM, about 30 nM to about 95 nM, about 30 nM to about 90 nM, about 30 nM to about 85 nM, about 30 nM to about 80 nM, about 30 nM to about 75 nM, about 30 nM to about 70 nM, about 30 nM to about 65 nM, about 30 nM to about 60 nM, about 30 nM to about 55 nM, about 30 nM to about 50 nM, about 30 nM to about 45 nM, about 30 nM to about 40 nM, about 30 nM to about 35 nM, about 40 nM to about 5 μM, about 40 nM to about 2 μM, about 40 nM to about 1 μM, about 40 nM to about 500 nM, about 40 nM to about 250 nM, about 40 nM to about 240 nM, about 40 nM to about 230 nM, about 40 nM to about 220 nM, about 40 nM to about 210 nM, about 40 nM to about 200 nM, about 40 nM to about 190 nM, about 40 nM to about 180 nM, about 40 nM to about 170 nM, about 40 nM to about 160 nM, about 40 nM to about 150 nM, about 40 nM to about 140 nM, about 40 nM to about 130 nM, about 40 nM to about 120 nM, about 40 nM to about 110 nM, about 40 nM to about 100 nM, about 40 nM to about 95 nM, about 40 nM to about 90 nM, about 40 nM to about 85 nM, about 40 nM to about 80 nM, about 40 nM to about 75 nM, about 40 nM to about 70 nM, about 40 nM to about 65 nM, about 40 nM to about 60 nM, about 40 nM to about 55 nM, about 40 nM to about 50 nM, about 40 nM to about 45 nM, about 50 nM to about 5 μM, about 50 nM to about 2 μM, about 50 nM to about 1 μM, about 50 nM to about 500 nM, about 50 nM to about 250 nM, about 50 nM to about 240 nM, about 50 nM to about 230 nM, about 50 nM to about 220 nM, about 50 nM to about 210 nM, about 50 nM to about 200 nM, about 50 nM to about 190 nM, about 50 nM to about 180 nM, about 50 nM to about 170 nM, about 50 nM to about 160 nM, about 50 nM to about 150 nM, about 50 nM to about 140 nM, about 50 nM to about 130 nM, about 50 nM to about 120 nM, about 50 nM to about 110 nM, about 50 nM to about 100 nM, about 50 nM to about 95 nM, about 50 nM to about 90 nM, about 50 nM to about 85 nM, about 50 nM to about 80 nM, about 50 nM to about 75 nM, about 50 nM to about 70 nM, about 50 nM to about 65 nM, about 50 nM to about 60 nM, about 50 nM to about 55 nM, about 60 nM to about 5 μM, about 60 nM to about 2 μM, about 60 nM to about 1 μM, about 60 nM to about 500 nM, about 60 nM to about 250 nM, about 60 nM to about 240 nM, about 60 nM to about 230 nM, about 60 nM to about 220 nM, about 60 nM to about 210 nM, about 60 nM to about 200 nM, about 60 nM to about 190 nM, about 60 nM to about 180 nM, about 60 nM to about 170 nM, about 60 nM to about 160 nM, about 60 nM to about 150 nM, about 60 nM to about 140 nM, about 60 nM to about 130 nM, about 60 nM to about 120 nM, about 60 nM to about 110 nM, about 60 nM to about 100 nM, about 60 nM to about 95 nM, about 60 nM to about 90 nM, about 60 nM to about 85 nM, about 60 nM to about 80 nM, about 60 nM to about 75 nM, about 60 nM to about 70 nM, about 60 nM to about 65 nM, about 70 nM to about 5 μM, about 70 nM to about 2 μM, about 70 nM to about 1 μM, about 70 nM to about 500 nM, about 70 nM to about 250 nM, about 70 nM to about 240 nM, about 70 nM to about 230 nM, about 70 nM to about 220 nM, about 70 nM to about 210 nM, about 70 nM to about 200 nM, about 70 nM to about 190 nM, about 70 nM to about 180 nM, about 70 nM to about 170 nM, about 70 nM to about 160 nM, about 70 nM to about 150 nM, about 70 nM to about 140 nM, about 70 nM to about 130 nM, about 70 nM to about 120 nM, about 70 nM to about 110 nM, about 70 nM to about 100 nM, about 70 nM to about 95 nM, about 70 nM to about 90 nM, about 70 nM to about 85 nM, about 70 nM to about 80 nM, about 70 nM to about 75 nM, about 80 nM to about 5 μM, about 80 nM to about 2 μM, about 80 nM to about 1 pM, about 80 nM to about 500 nM, about 80 nM to about 250 nM, about 80 nM to about 240 nM, about 80 nM to about 230 nM, about 80 nM to about 220 nM, about 80 nM to about 210 nM, about 80 nM to about 200 nM, about 80 nM to about 190 nM, about 80 nM to about 180 nM, about 80 nM to about 170 nM, about 80 nM to about 160 nM, about 80 nM to about 150 nM, about 80 nM to about 140 nM, about 80 nM to about 130 nM, about 80 nM to about 120 nM, about 80 nM to about 110 nM, about 80 nM to about 100 nM, about 80 nM to about 95 nM, about 80 nM to about 90 nM, about 80 nM to about 85 nM, about 90 nM to about 5 μM, about 90 nM to about 2 μM, about 90 mM to about 1 μM, about 90 nM to about 500 nM, about 90 nM to about 250 nM, about 90 nM to about 240 nM, about 90 nM to about 230 nM, about 90 nM to about 220 nM, about 90 nM to about 210 nM, about 90 nM to about 200 nM, about 90 nM to about 190 nM, about 90 nM to about 180 nM, about 90 nM to about 170 nM, about 90 nM to about 160 nM, about 90 nM to about 150 nM, about 90 nM to about 140 nM, about 90 nM to about 130 nM, about 90 nM to about 120 nM, about 90 nM to about 110 nM, about 90 nM to about 100 nM, about 90 nM to about 95 nM, about 100 nM to about 5 μM, about 100 nM to about 2 pM, about 100 nM to about 1 μM, about 100 nM to about 500 nM, about 100 nM to about 250 nM, about 100 nM to about 240 nM, about 100 nM to about 230 nM, about 100 nM to about 220 nM, about 100 nM to about 210 nM, about 100 nM to about 200 nM, about 100 nM to about 190 nM, about 100 nM to about 180 nM, about 100 nM to about 170 nM, about 100 nM to about 160 nM, about 100 nM to about 150 nM, about 100 nM to about 140 nM, about 100 nM to about 130 nM, about 100 nM to about 120 nM, about 100 nM to about 110 nM, about 110 nM to about 5 μM, about 110 nM to about 2 μM, about 110 nM to about 1 μM, about 110 nM to about 500 nM, about 110 nM to about 250 nM, about 110 nM to about 240 nM, about 110 nM to about 230 nM, about 110 nM to about 220 nM, about 110 nM to about 210 nM, about 110 nM to about 200 nM, about 110 nM to about 190 nM, about 110 nM to about 180 nM, about 110 nM to about 170 nM, about 110 nM to about 160 nM, about 110 nM to about 150 nM, about 110 nM to about 140 nM, about 110 nM to about 130 nM, about 110 nM to about 120 nM, about 120 nM to about 5 μM, about 120 nM to about 2 μM, about 120 nM to about 1 μM, about 120 nM to about 500 nM, about 120 nM to about 250 nM, about 120 nM to about 240 nM, about 120 nM to about 230 nM, about 120 nM to about 220 nM, about 120 nM to about 210 nM, about 120 nM to about 200 nM, about 120 nM to about 190 nM, about 120 nM to about 180 nM, about 120 nM to about 170 nM, about 120 nM to about 160 nM, about 120 nM to about 150 nM, about 120 nM to about 140 nM, about 120 nM to about 130 nM, about 130 nM to about 5 μM, about 130 nM to about 2 μM, about 130 nM to about 1 μM, about 130 nM to about 500 nM, about 130 nM to about 250 nM, about 130 nM to about 240 nM, about 130 nM to about 230 nM, about 130 nM to about 220 nM, about 130 nM to about 210 nM, about 130 nM to about 200 nM, about 130 nM to about 190 nM, about 130 nM to about 180 nM, about 130 nM to about 170 nM, about 130 nM to about 160 nM, about 130 nM to about 150 nM, about 130 nM to about 140 nM, about 140 nM to about 5 μM, about 140 nM to about 2 μM, about 140 nM to about 1 μM, about 140 nM to about 500 nM, about 140 nM to about 250 nM, about 140 nM to about 240 nM, about 140 nM to about 230 nM, about 140 nM to about 220 nM, about 140 nM to about 210 nM, about 140 nM to about 200 nM, about 140 nM to about 190 nM, about 140 nM to about 180 nM, about 140 nM to about 170 nM, about 140 nM to about 160 nM, about 140 nM to about 150 nM, about 150 nM to about 5 μM, about 150 nM to about 2 μM, about 150 nM to about 1 μM, about 150 nM to about 500 nM, about 150 nM to about 250 nM, about 150 nM to about 240 nM, about 150 nM to about 230 nM, about 150 nM to about 220 nM, about 150 nM to about 210 nM, about 150 nM to about 200 nM, about 150 nM to about 190 nM, about 150 nM to about 180 nM, about 150 nM to about 170 nM, about 150 nM to about 160 nM, about 160 nM to about 5 μM, about 160 nM to about 2 μM, about 160 nM to about 1 μM, about 160 nM to about 500 nM, about 160 nM to about 250 nM, about 160 nM to about 240 nM, about 160 nM to about 230 nM, about 160 nM to about 220 nM, about 160 nM to about 210 nM, about 160 nM to about 200 nM, about 160 nM to about 190 nM, about 160 nM to about 180 nM, about 160 nM to about 170 nM, about 170 nM to about 5 μM, about 170 nM to about 2 μM, about 170 nM to about 1 μM, about 170 nM to about 500 nM, about 170 nM to about 250 nM, about 170 nM to about 240 nM, about 170 nM to about 230 nM, about 170 nM to about 220 nM, about 170 nM to about 210 nM, about 170 nM to about 200 nM, about 170 nM to about 190 nM, about 170 nM to about 180 nM, about 180 nM to about 5 μM, about 180 nM to about 2 μM, about 180 nM to about 1 μM, about 180 nM to about 500 nM, about 180 nM to about 250 nM, about 180 nM to about 240 nM, about 180 nM to about 230 nM, about 180 nM to about 220 nM, about 180 nM to about 210 nM, about 180 nM to about 200 nM, about 180 nM to about 190 nM, about 190 nM to about 5 μM, about 190 nM to about 2 μM, about 190 nM to about 1 μM, about 190 nM to about 500 nM, about 190 nM to about 250 nM, about 190 nM to about 240 nM, about 190 nM to about 230 nM, about 190 nM to about 220 nM, about 190 nM to about 210 nM, about 190 nM to about 200 nM, about 200 nM to about 5 μM, about 200 nM to about 2 μM, about 200 nM to about 1 μM, about 200 nM to about 500 nM, about 200 nM to about 250 nM, about 200 nM to about 240 nM, about 200 nM to about 230 nM, about 200 nM to about 220 nM, about 200 nM to about 210 nM, about 210 nM to about 5 μM, about 210 nM to about 2 μM, about 210 nM to about 1 μM, about 210 nM to about 500 nM, about 210 nM to about 250 nM, about 210 nM to about 240 nM, about 210 nM to about 230 nM, about 210 nM to about 220 nM, about 220 nM to about 5 μM, about 220 nM to about 2 μM, about 220 nM to about 1 μM, about 220 nM to about 500 nM, about 220 nM to about 250 nM, about 220 nM to about 240 nM, about 220 nM to about 230 nM, about 230 nM to about 5 μM, about 230 nM to about 2 μM, about 230 nM to about 1 μM, about 230 nM to about 500 nM, about 230 nM to about 250 nM, about 230 nM to about 240 nM, about 240 nM to about 5 μM, about 240 nM to about 2 μM, about 240 nM to about 1 μM, about 240 nM to about 500 nM, about 240 nM to about 250 nM, about 250 nM to about 5 μM, about 250 nM to about 2 μM, about 250 nM to about 1 μM, about 250 nM to about 500 nM, about 500 nM to about 5 μM, about 500 nM to about 2 μM, about 500 nM to about 1 μM, about 1 pM to about 5 μM, about 1 μM to about 2 μM, or about 2 μM to about 5 μM).

In some embodiments of any of the ABPCs described herein, the K_{D} of the first antigen-binding domain (and optionally, the second antigen-binding domain, if present) at a pH of about 4.0 to about 6.5 (e.g., any of the subranges of this range described herein) can be greater than 1 nM (e.g., between about 1 nM to about 1 mM, about 1 nM to about 900 μM, about 1 nM to about 800 μM, about 1 nM to about 700 μM, about 1 nM to about 600 μM, about 1 nM to about 500 μM, about 1 nM to about 400 μM, about 1 nM to about 300 μM, about 1 nM to about 200 μM, about 1 nM to about 100 μM, about 1 nM to about 90 μM, about 1 nM to about 80 μM, about 1 nM to about 70 μM, about 1 nM to about 60 μM, about 1 nM to about 50 μM, about 1 nM to about 40 μM, about 1 nM to about 30 μM, about 1 nM to about 20 μM, about 1 nM to about 10 μM, about 1 nM to about 5 μM, about 1 nM to about 4 μM, about 1 nM to about 2 μM, about 1 nM to about 1 μM, about 1 nM to about 900 nM, about 1 nM to about 800 nM, about 1 nM to about 700 nM, about 1 nM to about 600 nM, about 1 nM to about 500 nM, about 1 nM to about 400 nM, about 1 nM to about 300 nM, about 1 nM to about 200 nM, about 1 nM to about 100 nM, about 1 nM to about 90 nM, about 1 nM to about 80 nM, about 1 nM to about 70 nM, about 1 nM to about 60 nM, about 1 nM to about 50 nM, about 1 nM to about 40 nM, about 1 nM to about 30 nM, about 2 nM to about 1 mM, about 2 nM to about 900 μM, about 2 nM to about 800 μM, about 2 nM to about 700 μM, about 2 nM to about 600 μM, about 2 nM to about 500 μM, about 2 nM to about 400 μM, about 2 nM to about 300 μM, about 2 nM to about 200 μM, about 2 nM to about 100 μM, about 2 nM to about 90 μM, about 2 nM to about 80 μM, about 2 nM to about 70 μM, about 2 nM to about 60 μM, about 2 nM to about 50 μM, about 2 nM to about 40 μM, about 2 nM to about 30 μM, about 2 nM to about 20 μM, about 2 nM to about 10 μM, about 2 nM to about 5 μM, about 2 nM to about 4 μM, about 2 nM to about 2 μM, about 2 nM to about 1 μM, about 2 nM to about 900 nM, about 2 nM to about 800 nM, about 2 nM to about 700 nM, about 2 nM to about 600 nM, about 2 nM to about 500 nM, about 2 nM to about 400 nM, about 2 nM to about 300 nM, about 2 nM to about 200 nM, about 2 nM to about 100 nM, about 2 nM to about 90 nM, about 2 nM to about 80 nM, about 2 nM to about 70 nM, about 2 nM to about 60 nM, about 2 nM to about 50 nM, about 2 nM to about 40 nM, about 2 nM to about 30 nM, about 5 nM to about 1 mM, about 5 nM to about 900 μM, about 5 nM to about 800 μM, about 5 nM to about 700 μM, about 5 nM to about 600 μM, about 5 nM to about 500 μM, about 5 nM to about 400 μM, about 5 nM to about 300 μM, about 5 nM to about 200 μM, about 5 nM to about 100 μM, about 5 nM to about 90 μM, about 5 nM to about 80 μM, about 5 nM to about 70 μM, about 5 nM to about 60 μM, about 5 nM to about 50 μM, about 5 nM to about 40 μM, about 5 nM to about 30 μM, about 5 nM to about 20 μM, about 5 nM to about 10 μM, about 5 nM to about 5 μM, about 5 nM to about 4 μM, about 5 nM to about 2 μM, about 5 nM to about 1 μM, about 5 nM to about 900 nM, about 5 nM to about 800 nM, about 5 nM to about 700 nM, about 5 nM to about 600 nM, about 5 nM to about 500 nM, about 5 nM to about 400 nM, about 5 nM to about 300 nM, about 5 nM to about 200 nM, about 5 nM to about 100 nM, about 5 nM to about 90 nM, about 5 nM to about 80 nM, about 5 nM to about 70 nM, about 5 nM to about 60 nM, about 5 nM to about 50 nM, about 5 nM to about 40 nM, about 5 nM to about 30 nM, about 10 nM to about 1 mM, about 10 nM to about 900 μM, about 10 nM to about 800 μM, about 10 nM to about 700 μM, about 10 nM to about 600 μM, about 10 nM to about 500 μM, about 10 nM to about 400 μM, about 10 nM to about 300 μM, about 10 nM to about 200 μM, about 10 nM to about 100 μM, about 10 nM to about 90 μM, about 10 nM to about 80 μM, about 10 nM to about 70 μM, about 10 nM to about 60 μM, about 10 nM to about 50 μM, about 10 nM to about 40 μM, about 10 nM to about 30 μM, about 10 nM to about 20 μM, about 10 nM to about 10 μM, about 10 nM to about 5 μM, about 10 nM to about 4 μM, about 10 nM to about 2 μM, about 10 nM to about 1 μM, about 10 nM to about 900 nM, about 10 nM to about 800 nM, about 10 nM to about 700 nM, about 10 nM to about 600 nM, about 10 nM to about 500 nM, about 10 nM to about 400 nM, about 10 nM to about 300 nM, about 10 nM to about 200 nM, about 10 nM to about 100 nM, about 10 nM to about 90 nM, about 10 nM to about 80 nM, about 10 nM to about 70 nM, about 10 nM to about 60 nM, about 10 nM to about 50 nM, about 10 nM to about 40 nM, about 10 nM to about 30 nM, about 20 nM to about 1 mM, about 20 nM to about 900 μM, about 20 nM to about 800 μM, about 20 nM to about 700 μM, about 20 nM to about 600 μM, about 20 nM to about 500 μM, about 20 nM to about 400 μM, about 20 nM to about 300 μM, about 20 nM to about 200 μM, about 20 nM to about 100 μM, about 20 nM to about 90 μM, about 20 nM to about 80 μM, about 20 nM to about 70 μM, about 20 nM to about 60 μM, about 20 nM to about 50 μM, about 20 nM to about 40 μM, about 20 nM to about 30 μM, about 20 nM to about 20 μM, about 20 nM to about 10 μM, about 20 nM to about 5 μM, about 20 nM to about 4 μM, about 20 nM to about 2 μM, about 20 nM to about 1 μM, about 20 nM to about 900 nM, about 20 nM to about 800 nM, about 20 nM to about 700 nM, about 20 nM to about 600 nM, about 20 nM to about 500 nM, about 20 nM to about 400 nM, about 20 nM to about 300 nM, about 20 nM to about 200 nM, about 20 nM to about 100 nM, about 20 nM to about 90 nM, about 20 nM to about 80 nM, about 20 nM to about 70 nM, about 20 nM to about 60 nM, about 20 nM to about 50 nM, about 20 nM to about 40 nM, about 20 nM to about 30 nM; about 1 μM to about 1 mM, about 1 μM to about 900 μM, about 1 μM to about 800 μM, about 1 μM to about 700 μM, about 1 μM to about 600 μM, about 1 μM to about 500 μM, about 1 μM to about 400 μM, about 1 μM to about 300 μM, about 1 μM to about 200 μM, about 1 μM to about 100 μM, about 1 μM to about 90 μM, about 1 μM to about 80 μM, about 1 μM to about 70 μM, about 1 μM to about 60 μM, about 1 μM to about 50 μM, about 1 μM to about 40 μM, about 1 μM to about 30 μM, about 1 μM to about 20 μM, about 1 μM to about 10 μM, about 1 μM to about 5 μM, about 1 μM to about 4 μM, about 1 μM to about 3 μM, about 1 μM to about 2 μM, about 2 μM to about 1 mM, about 2 μM to about 900 μM, about 2 μM to about 800 μM, about 2 μM to about 700 μM, about 2 μM to about 600 μM, about 2 μM to about 500 μM, about 2 μM to about 400 μM, about 2 μM to about 300 μM, about 2 μM to about 200 μM, about 2 μM to about 100 μM, about 2 μM to about 90 μM, about 2 μM to about 80 μM, about 2 μM to about 70 μM, about 2 μM to about 60 μM, about 2 μM to about 50 μM, about 2 μM to about 40 μM, about 2 μM to about 30 μM, about 2 μM to about 20 μM, about 2 μM to about 10 μM, about 2 μM to about 5 μM, about 2 μM to about 4 μM, about 2 μM to about 3 μM, about 5 μM to about 1 mM, about 5 μM to about 900 μM, about 5 μM to about 800 μM, about 5 μM to about 700 μM, about 5 μM to about 600 μM, about 5 μM to about 500 μM, about 5 μM to about 400 μM, about 5 μM to about 300 μM, about 5 μM to about 200 μM, about 5 μM to about 100 μM, about 5 μM to about 90 μM, about 5 μM to about 80 μM, about 5 μM to about 70 μM, about 5 μM to about 60 μM, about 5 μM to about 50 μM, about 5 μM to about 40 μM, about 5 μM to about 30 μM, about 5 μM to about 20 μM, about 5 μM to about 10 μM, about 10 μM to about 1 mM, about 10 μM to about 900 μM, about 10 μM to about 800 μM, about 10 μM to about 700 μM, about 10 μM to about 600 μM, about 10 μM to about 500 μM, about 10 μM to about 400 μM, about 10 μM to about 300 μM, about 10 μM to about 200 μM, about 10 μM to about 100 μM, about 10 μM to about 90 μM, about 10 μM to about 80 μM, about 10 μM to about 70 μM, about 10 μM to about 60 μM, about 10 μM to about 50 μM, about 10 μM to about 40 μM, about 10 μM to about 30 μM, about 10 μM to about 20 μM, about 20 μM to about 1 mM, about 20 μM to about 900 μM, about 20 μM to about 800 μM, about 20 μM to about 700 μM, about 20 μM to about 600 μM, about 20 μM to about 500 μM, about 20 μM to about 400 μM, about 20 μM to about 300 μM, about 20 μM to about 200 μM, about 20 μM to about 100 μM, about 20 μM to about 90 μM, about 20 μM to about 80 μM, about 20 μM to about 70 μM, about 20 μM to about 60 μM, about 20 μM to about 50 μM, about 20 μM to about 40 μM, about 20 μM to about 30 μM, about 30 μM to about 1 mM, about 30 μM to about 900 μM, about 30 μM to about 800 μM, about 30 μM to about 700 μM, about 30 μM to about 600 μM, about 30 μM to about 500 μM, about 30 μM to about 400 μM, about 30 μM to about 300 μM, about 30 μM to about 200 μM, about 30 μM to about 100 μM, about 30 μM to about 90 μM, about 30 μM to about 80 μM, about 30 μM to about 70 μM, about 30 μM to about 60 μM, about 30 μM to about 50 μM, about 30 μM to about 40 μM, about 40 μM to about 1 mM, about 40 μM to about 900 μM, about 40 μM to about 800 μM, about 40 μM to about 700 μM, about 40 μM to about 600 μM, about 40 μM to about 500 μM, about 40 μM to about 400 μM, about 40 μM to about 300 μM, about 40 μM to about 200 μM, about 40 μM to about 100 μM, about 40 μM to about 90 μM, about 40 μM to about 80 μM, about 40 μM to about 70 μM, about 40 μM to about 60 μM, about 40 μM to about 50 μM, about 50 μM to about 1 mM, about 50 μM to about 900 μM, about 50 μM to about 800 μM, about 50 μM to about 700 μM, about 50 μM to about 600 μM, about 50 μM to about 500 μM, about 50 μM to about 400 μM, about 50 μM to about 300 μM, about 50 μM to about 200 μM, about 50 μM to about 100 μM, about 50 μM to about 90 μM, about 50 μM to about 80 μM, about 50 μM to about 70 μM, about 50 μM to about 60 μM, about 60 μM to about 1 mM, about 60 μM to about 900 μM, about 60 μM to about 800 μM, about 60 μM to about 700 μM, about 60 μM to about 600 μM, about 60 μM to about 500 μM, about 60 μM to about 400 μM, about 60 μM to about 300 μM, about 60 μM to about 200 μM, about 60 μM to about 100 μM, about 60 μM to about 90 μM, about 60 μM to about 80 μM, about 60 μM to about 70 μM, about 70 μM to about 1 mM, about 70 μM to about 900 μM, about 70 μM to about 800 μM, about 70 μM to about 700 μM, about 70 μM to about 600 μM, about 70 μM to about 500 μM, about 70 μM to about 400 μM, about 70 μM to about 300 μM, about 70 μM to about 200 μM, about 70 μM to about 100 μM, about 70 μM to about 90 μM, about 70 μM to about 80 μM, about 80 μM to about 1 mM, about 80 μM to about 900 μM, about 80 μM to about 800 μM, about 80 μM to about 700 μM, about 80 μM to about 600 μM, about 80 μM to about 500 μM, about 80 μM to about 400 μM, about 80 μM to about 300 μM, about 80 μM to about 200 μM, about 80 μM to about 100 μM, about 80 μM to about 90 μM, about 90 μM to about 1 mM, about 90 μM to about 900 μM, about 90 μM to about 800 μM, about 90 μM to about 700 μM, about 90 μM to about 600 μM, about 90 μM to about 500 μM, about 90 μM to about 400 μM, about 90 μM to about 300 μM, about 90 μM to about 200 μM, about 90 μM to about 100 μM, about 100 μM to about 1 mM, about 100 μM to about 900 μM, about 100 μM to about 800 μM, about 100 μM to about 700 μM, about 100 μM to about 600 μM, about 100 μM to about 500 μM, about 100 μM to about 400 μM, about 100 μM to about 300 μM, about 100 μM to about 200 μM, about 200 μM to about 1 mM, about 200 μM to about 900 μM, about 200 μM to about 800 μM, about 200 μM to about 700 μM, about 200 μM to about 600 μM, about 200 μM to about 500 μM, about 200 μM to about 400 μM, about 200 μM to about 300 μM, about 300 μM to about 1 mM, about 300 μM to about 900 μM, about 300 μM to about 800 μM, about 300 μM to about 700 μM, about 300 μM to about 600 μM, about 300 μM to about 500 μM, about 300 μM to about 400 μM, about 400 μM to about 1 mM, about 400 μM to about 900 μM, about 400 μM to about 800 μM, about 400 μM to about 700 μM, about 400 μM to about 600 μM, about 400 μM to about 500 μM, about 500 μM to about 1 mM, about 500 μM to about 900 μM, about 500 μM to about 800 μM, about 500 μM to about 700 μM, about 500 μM to about 600 μM, about 600 μM to about 1 mM, about 600 μM to about 900 μM, about 600 μM to about 800 μM, about 600 μM to about 700 μM, about 700 μM to about 1 mM, about 700 μM to about 900 μM, about 700 μM to about 800 μM, about 800 μM to about 1 mM, about 800 μM to about 900 μM, or about 900 μM to about 1 mM).

A variety of different methods known in the art can be used to determine the K_{D} values of any of the antigen-binding protein constructs described herein (e.g., an electrophoretic mobility shift assay, a filter binding assay, surface plasmon resonance, a biomolecular binding kinetics assay, in vitro binding assay on antigen-expressing cells, etc.).

In some examples of any of the ABPCs described herein, the half-life of the ABPC in vivo is decreased (e.g., a detectable decrease) (e.g., at least a 1% decrease, at least a 5% decrease, at least a 10% decrease, at least a 15% decrease, at least a 20% decrease, at least a 25% decrease, at least a 30% decrease, at least a 35% decrease, at least a 40% decrease, at least a 45% decrease, at least a 50% decrease, at least a 55% decrease, at least a 60% decrease, at least a 65% decrease, at least a 70% decrease, at least a 75% decrease, at least a 80% decrease, at least a 85% decrease, at least a 90% decrease, at least a 95% decrease, or at least a 99% decrease, or about a 1% decrease to about a 99% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 99% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 99% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 99% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 99% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 99% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 99% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 99% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 99% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 99% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 99% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 99% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 99% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 99% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 99% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 99% decrease, about a 75% decrease to about a 95% decrease, about a 75% decrease to about a 90% decrease, about a 75% decrease to about a 85% decrease, about a 75% decrease to about a 80% decrease, about a 80% decrease to about a 99% decrease, about a 80% decrease to about a 95% decrease, about a 80% decrease to about a 90% decrease, about a 80% decrease to about a 85% decrease, about a 85% decrease to about a 99% decrease, about a 85% decrease to about a 95% decrease, about a 85% decrease to about a 90% decrease, about a 90% decrease to about a 99% decrease, about a 90% decrease to about a 95% decrease, or about a 95% decrease to about a 99% decrease) as compared to the half-life of a control ABPC (e.g., any of the exemplary control ABPCs described herein).

### Conjugation

In some embodiments, the ABPCs provided herein can be conjugated to a drug (e.g., a chemotherapeutic drug, a small molecule), a toxin, or a radioisotope. Non-limiting examples of drugs, toxins, and radioisotopes (e.g., known to be useful for the treatment of cancer) are known in the art.

In some embodiments, at least one polypeptide of any of the ABPCs described herein is conjugated to the toxin, the radioisotope, or the drug via a cleavable linker. In some embodiments, the cleavable linker includes a protease cleavage site. In some embodiments, the cleavable linker is cleaved on the ABPC once it is transported to the lysosome or late endosome by the target mammalian cell. In some embodiments, cleavage of the linker functionally activates the drug or toxin.

In some embodiments, at least one polypeptide of any of the ABPCs described herein is conjugated to the toxin, the radioisotope, or the drug via a non-cleavable linker. In some embodiments, the conjugated toxin, radioisotope, or drug is released during lysosomal and/or late endosomal degradation of the ABPC.

Non-limiting examples of cleavable linkers include: hydrazone linkers, peptide linkers, disulfide linkers, and thioether linkers. See, e.g., Carter et al., Cancer J. 14(3):154-169, 2008; Sanderson et al., Clin. Cancer Res. 11(2 Ptl):843-852, 2005; Chari et al., Acc. Chem. Res. 41(1):98-107, 2008; Oflazoglu et al., Clin. Cancer Res. 14(19): 6171-6180, 2008; and Lu et al., Int. J. Mol. Sci. 17(4): 561, 2016.

Non-limiting examples of non-cleavable linkers include: maleimide alkane-linkers and meleimide cyclohexane linker (MMC) (see, e.g., those described in McCombs et al., AAPSJ. 17(2):339-351, 2015).

In some embodiments, any of the ABPCs described herein is cytotoxic or cytostatic to the target mammalian cell.

### Expression of an Antigen-Binding Protein Construct in a Cell

Also provided herein are methods of generating a recombinant cell that expresses an ABPC (e.g., any of the ABPCs described herein) that include: introducing into a cell a nucleic acid encoding the ABPC to produce a recombinant cell; and culturing the recombinant cell under conditions sufficient for the expression of the ABPC. In some embodiments, the introducing step includes introducing into a cell an expression vector including a nucleic acid encoding the ABPC to produce a recombinant cell.

Any of the ABPCs described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the ABPCs from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Methods of Treatment

Provided herein are methods of treating a cancer characterized by having a population of cancer cells that have DLL3 or an epitope of DLL3 presented on their surface, that include: administering a therapeutically effective amount of any of the pharmaceutical compositions described herein or any of the ABPCs described herein to a subject identified as having a cancer characterized by having the population of cancer cells.

Also provided herein are methods of reducing the volume of a tumor in a subject, wherein the tumor is characterized by having a population of cancer cells that have DLL3 or an epitope of DLL3 presented on their surface, that include: administering a therapeutically effective amount of any of the pharmaceutical compositions described herein or any of the ABPCs described herein to a subject identified as having a cancer characterized by having the population of cancer cells. In some embodiments of any of the methods described herein, the volume of at least one (e.g., 1, 2, 3, 4, or 5) tumor (e.g., solid tumor) or tumor location (e.g., a site of metastasis) is reduced (e.g., a detectable reduction) by at least 1%, at least 2%, at least 3%, at least 4%, at least 5%, at least 6%, at least 8%, at least 10%, at least 12%, at least 14%, at least 16%, at least 18%, at least 20%, at least 22%, at least 24%, at least 26%, at least 28%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%) reduced as compared to the size of the at least one tumor (e.g., solid tumor) before administration of the ABPC.

Also provided herein are methods of inducing cell death in a cancer cell in a subject, wherein the cancer cell has DLL3 or an epitope of DLL3 presented on its surface, that include: administering a therapeutically effective amount of any of the pharmaceutical compositions of described herein or any of the ABPCs described herein to a subject identified as having a cancer characterized as having the population of cancer cells. In some embodiments, the cell death that is induced is necrosis. In some embodiments, the cell death that is induced is apoptosis.

In some embodiments of any of the methods described herein, the cancer is a primary tumor.

In some embodiments of any of the methods described herein, the cancer is a metastasis.

In some embodiments of any of the methods described herein, the cancer is a non-T-cell-infiltrating tumor. In some embodiments of any of the methods described herein, the cancer is a T-cell-infiltrating tumor.

Provided herein are methods of decreasing the risk of developing a metastasis or decreasing the risk of developing an additional metastasis in a subject having a cancer, wherein the cancer is characterized by having a population of cancer cells that have DLL3 or an epitope of DLL3 presented on their surface, that include: administering a therapeutically effective amount of any of the pharmaceutical compositions of described herein or any of the ABPCs described herein to a subject identified as having a cancer characterized as having the population of cancer cells. In some embodiments, the risk of developing a metastasis or the risk of developing an additional metastasis is decreased (e.g., a detectable decrease) by at least 1%, by at least 2%, at least 3%, at least 4%, at least 5%, at least 6%, at least 8%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in the subject as compared to the risk of a subject having a similar cancer, but administered no treatment or a treatment that does not include the administration of any of the ABPCs described herein.

In some embodiments of any of the methods described herein, the cancer is a non-T-cell-infiltrating tumor. In some embodiments of any of the methods described herein, the cancer is a T-cell-infiltrating tumor. In some embodiments of any of the methods described herein, the cellular compartment is part of the endosomal/lysosomal pathway. In some embodiments of any of the methods described herein, the cellular compartment is an endosome.

The term “subject” refers to any mammal. In some embodiments, the subject or “subject suitable for treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

As used herein, treating includes reducing the number, frequency, or severity of one or more (e.g., two, three, four, or five) signs or symptoms of a cancer in a patient having a cancer (e.g., any of the cancers described herein). For example, treatment can reducing cancer progression, reduce the severity of a cancer, or reduce the risk of re-occurrence of a cancer in a subject having the cancer.

Provided herein are methods of inhibiting the growth of a solid tumor in a subject (e.g., any of the subjects described herein) that include administering to the subject a therapeutically effective amount of any of the ABPCs described herein or any of the pharmaceutical compositions described herein (e.g., as compared to the growth of the solid tumor in the subject prior to treatment or the growth of a similar solid tumor in a different subject receiving a different treatment or receiving no treatment).

In some embodiments of any of the methods described herein, the growth of a solid tumor is primary growth of a solid tumor. In some embodiments of any of the methods described herein, the growth of a solid tumor is recurrent growth of a solid tumor. In some embodiments of any of the methods described herein, the growth of a solid tumor is metastatic growth of a solid tumor. In some embodiments, treatment results in about a 1% decrease to about 99% decrease (or any of the subranges of this range described herein) in the growth of a solid tumor in the subject (e.g., as compared to the growth of the solid tumor in the subject prior to treatment or the growth of a similar solid tumor in a different subject receiving a different treatment or receiving no treatment). The growth of a solid tumor in a subject can be assessed by a variety of different imaging methods, e.g., positron emission tomograph, X-ray computed tomography, computed axial tomography, and magnetic resonance imaging.

Also provided herein are methods of decreasing the risk of developing a metastasis or developing an additional metastasis over a period of time in a subject identified as having a cancer (e.g., any of the exemplary cancers described herein) that include administering to the subject a therapeutically effective amount of any of the proteins described herein or any of the pharmaceutical compositions described herein (e.g., as compared to a subject having a similar cancer and receiving a different treatment or receiving no treatment). In some embodiments of any of the methods described herein, the metastasis or additional metastasis is one or more to a bone, lymph nodes, brain, lung, liver, skin, chest wall including bone, cartilage and soft tissue, abdominal cavity, contralateral breast, soft tissue, muscle, bone marrow, ovaries, adrenal glands, and pancreas.

In some embodiments of any of the methods described herein, the period of time is about 1 month to about 3 years (e.g., about 1 month to about 2.5 years, about 1 month to about 2 years, about 2 months to about 1.5 years, about 1 month to about 1 year, about 1 month to about 10 months, about 1 month to about 8 months, about 1 month to about 6 months, about 1 month to about 5 months, about 1 month to about 4 months, about 1 month to about 3 months, about 1 month to about 2 months, about 2 months to about 3 years, about 2 months to about 2.5 years, about 2 months to about 2 years, about 2 months to about 1.5 years, about 2 months to about 1 year, about 2 months to about 10 months, about 2 months to about 8 months, about 2 months to about 6 months, about 2 months to about 5 months, about 2 months to about 4 months, about 2 months to about 3 months, about 3 months to about 3 years, about 3 months to about 2.5 years, about 3 months to about 2 years, about 3 months to about 1.5 years, about 3 months to about 1 year, about 3 months to about 10 months, about 3 months to about 8 months, about 3 months to about 6 months, about 3 months to about 5 months, about 3 months to about 4 months, about 4 months to about 3 years, about 4 months to about 2.5 years, about 4 months to about 2 years, about 4 months to about 1.5 years, about 4 months to about 1 year, about 4 months to about 10 months, about 4 months to about 8 months, about 4 months to about 6 months, about 4 months to about 5 months, about 5 months to about 3 years, about 5 months to about 2.5 years, about 5 months to about 2 years, about 5 months to about 1.5 years, about 5 months to about 1 year, about 5 months to about 10 months, about 5 months to about 8 months, about 5 months to about 6 months, about 6 months to about 3 years, about 6 months to about 2.5 years, about 6 months to about 2 years, about 6 months to about 1.5 years, about 6 months to about 1 year, about 6 months to about 10 months, about 6 months to about 8 months, about 8 months to about 3 years, about 8 months to about 2.5 years, about 8 months to about 2 years, about 8 months to about 1.5 years, about 8 months to about 1 year, about 8 months to about 10 months, about 10 months to about 3 years, about 10 months to about 2.5 years, about 10 months to about 2 years, about 10 months to about 1.5 years, about 10 months to about 1 year, about 1 year to about 3 years, about 1 year to about 2.5 years, about 1 year to about 2 years, about 1 year to about 1.5 years, about 1.5 years to about 3 years, about 1.5 years to about 2.5 years, about 1.5 years to about 2 years, about 2 years to about 3 years, about 2 years to about 2.5 years, or about 2.5 years to about 3 years).

In some embodiments, the risk of developing a metastasis or developing an additional metastasis over a period of time in a subject identified as having a cancer is decreased by about 1% to about 99% (e.g., or any of the subranges of this range described herein), e.g., as compared to the risk in a subject having a similar cancer receiving a different treatment or receiving no treatment.

Non-limiting examples of cancer include: acute lymphoblastic leukemia (ALL), acute myeloid leukemia (AML), adrenocortical carcinorna, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. Additional examples of cancer are known in the art.

In some embodiments, the patient is further administered one or more additional therapeutic agents (e.g., one or more of a chemotherapeutic agent, a recombinant cytokine or interleukin protein, a kinase inhibitor, and a checkpoint inhibitor). In some embodiments, the one or more additional therapeutic agents is administered to the patient at approximately the same time as any of the ABPCs described herein are administered to the patient. In some embodiments, the one or more additional therapeutic agents are administered to the patient after the administration of any of the ABPCs described herein to the patient. In some embodiments, the one or more additional therapeutic agents are administered to the patient before the administration of any of the ABPCs described herein to the patient.

In some embodiments of any of the methods described herein, the cancer is a solid cancer (e.g., breast cancer, prostate cancer, or non-small cell lung cancer).

### Compositions

Also provided herein are compositions (e.g., pharmaceutical compositions) that include at least one of any of the ABPCs described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the patient. A dosage of the pharmaceutical composition should provide a sufficient quantity of the ABPC to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

### Kits

Also provided herein are kits that include any of the ABPCs described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### Protein Constructs

Also provided are protein constructs (PCs) that include: a first antigen-binding domain that is capable of specifically binding DLL3 or an epitope of DLL3 presented on the surface of a target mammalian cell, where: (a) the dissociation rate of the first antigen-binding domain at a pH of about 7.0 to about 8.0 (or any of the subranges of this range described herein) is faster than the dissociation rate at a pH of about 4.0 to about 6.5 (or any of the subranges of this range described herein); and/or (b) the dissociation constant (K_{D}) of the first antigen-binding domain at a pH of about 7.0 to about 8.0 (or any of the subranges of this range) is greater than the K_{D} at a pH of about 4.0 to about 6.5.

Also provided herein are pharmaceutical compositions including any of the PCs described herein. Also provided herein are methods of treating a subject in need thereof that include administering a therapeutically effective amount of any of the PCs described herein to the subject.

### Methods of Improving pH Dependence of an Antigen-Binding Protein Construct

Also provided herein are methods of improving pH dependence of an antigen-binding protein construct, the method comprises providing a starting antigen-binding protein construct comprising an antigen-binding domain and introducing one or more histidine amino acid substitutions into one or more CDRs of the antigen-binding domain in the starting antigen-binding protein construct, wherein the method results in the generation of an antigen-binding protein construct having one or both of: (a) an increased (e.g., at least a 0.1-fold increase to about a 100-fold increase, or any of the subranges of this range described herein) ratio of the dissociation rate of the antigen-binding domain at a pH of about 4.0 to about 6.5 to the dissociation rate at a pH of about 7.0 to about 8.0, as compared to the starting antigen-binding protein construct, and (b) an increased (e.g., at least a 0.1-fold increase to about a 100-fold increase, or any of the subranges of this range described herein) ratio the dissociation constant (K_{D}) of the antigen-binding domain at a pH of about 4.0 to about 6.5 to the K_{D} at a pH of about 7.0 to about 8.0, as compared to the starting antigen-binding protein construct.

### Exemplary Embodiments

Embodiment 1 is a pharmaceutical composition comprising an effective amount of an antigen-binding protein construct (ABPC) comprising: a first antigen-binding domain that is capable of specifically binding DLL3 or an epitope of DLL3 presented on the surface of a target mammalian cell, wherein: (a) the dissociation rate of the first antigen-binding domain at a pH of about 4.0 to about 6.5 is faster than the dissociation rate at a pH of about 7.0 to about 8.0 or (b) the dissociation constant (KD) of the first antigen-binding domain at a pH of about 4.0 to about 6.5 is greater than the KD at a pH of about 7.0 to about 8.0.

Embodiment 2 is the pharmaceutical composition of embodiment 1, wherein the first antigen-binding domain comprises a heavy chain variable domain of rovalpituzumab with one or more amino acids substituted with a histidine.

Embodiment 3 is the pharmaceutical composition of embodiment 1, wherein the first antigen-binding domain comprises a light chain variable domain of rovalpituzumab with one or more amino acids substituted with a histidine.

Embodiment 4 is the pharmaceutical composition of embodiment 1, wherein the first antigen-binding domain comprises: a heavy chain variable domain of rovalpituzumab with one or more amino acids substituted with a histidine; and a light chain variable domain of rovalpituzumab with one or more amino acids substituted with a histidine.

Embodiment 5 is the pharmaceutical composition of embodiment 2 or 4, wherein the heavy chain variable domain of rovalpituzumab comprises SEQ ID NO: 1.

Embodiment 6 is the pharmaceutical composition of embodiment 3 or 4, wherein the light chain variable domain of rovalpituzumab comprises SEQ ID NO: 2.

Embodiment 7 is the pharmaceutical composition of embodiment 1, wherein the first antigen-binding domain comprises a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine.

Embodiment 8 is the pharmaceutical composition of embodiment 1, wherein the first DLL3-binding domain comprises a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 9 is the pharmaceutical composition of embodiment 1, wherein the first DLL3-binding domain comprises: a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine; and a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 10 is the pharmaceutical composition of embodiment 1, 2, or 7, wherein the first antigen-binding domain comprises a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106.

Embodiment 11 is the pharmaceutical composition of embodiment 1, 3, or 8, wherein the first antigen-binding domain comprises a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 12 is the pharmaceutical composition of embodiment 1, 2, or 7, wherein the first antigen-binding domain comprises a heavy chain variable domain that is at least 90% identical to SEQ ID NO: 1, wherein the heavy chain variable domain includes a histidine at two or more positions in SEQ ID NO: 1 selected from the group consisting of: 29, 31, 54, 55, and 105.

Embodiment 13 is the pharmaceutical composition of embodiment 1, 4, or 9, wherein the first antigen-binding domain comprises: a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 14 is the pharmaceutical composition of embodiment 1, wherein the first antigen-binding domain comprises a heavy chain variable domain of SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, or SEQ ID NO: 52.

Embodiment 15 is the pharmaceutical composition of embodiment 1 or 14, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 16 is the pharmaceutical composition of embodiment 1, wherein the first antigen-binding domain comprises a heavy chain variable domain SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91.

Embodiment 17 is the pharmaceutical composition of embodiment 16, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 18 is the pharmaceutical composition of any one of embodiments 1-17, wherein the ABPC is degraded in the target mammalian cell following internalization of the ABPC by the target mammalian cell.

Embodiment 19 is the pharmaceutical composition of any one of embodiments 1-18, wherein the ABPC further comprises a conjugated toxin, radioisotope, drug, or small molecule.

Embodiment 20 is the pharmaceutical composition of embodiment 19, wherein the composition provides for an increase in toxin liberation in the target mammalian cell as compared to a composition comprising the same amount of a control ABPC.

Embodiment 21 is the pharmaceutical composition of embodiment 20, wherein the composition provides for at least a 20% increase in toxin liberation in the target mammalian cell as compared to a composition comprising the same amount of a control ABPC.

Embodiment 22 is the pharmaceutical composition of embodiment 21, wherein the composition provides for at least a 50% increase in toxin liberation in the target mammalian cell as compared to a composition comprising the same amount of a control ABPC.

Embodiment 23 is the pharmaceutical composition of embodiment 20, wherein the composition provides for at least a 2-fold increase in toxin liberation in the target mammalian cell as compared to a composition comprising the same amount of a control ABPC.

Embodiment 24 is the pharmaceutical composition of embodiment 23, wherein the composition provides for at least a 5-fold increase in toxin liberation in the target mammalian cell as compared to a composition comprising the same amount of a control ABPC.

Embodiment 25 is the pharmaceutical composition of any one of embodiments 19-24, wherein the composition provides for an increase in target mammalian cell killing as compared to a composition comprising the same amount of a control ABPC.

Embodiment 26 is the pharmaceutical composition of embodiment 25, wherein the composition provides for at least a 20% increase in target mammalian cell killing as compared to a composition comprising the same amount of a control ABPC.

Embodiment 27 is the pharmaceutical composition of embodiment 26, wherein the composition provides for at least a 50% increase in target mammalian cell killing as compared to a composition comprising the same amount of a control ABPC.

Embodiment 28 is the pharmaceutical composition of embodiment 25, wherein the composition provides for at least a 2-fold increase in target mammalian cell killing as compared to a composition comprising the same amount of a control ABPC.

Embodiment 29 is the pharmaceutical composition of embodiment 28, wherein the composition provides for at least a 5-fold increase in target mammalian cell killing as compared to a composition comprising the same amount of a control ABPC.

Embodiment 30 is the pharmaceutical composition of any one of embodiments 1-29, wherein the composition provides for an increase in endolysosomal delivery in the target mammalian cell as compared to a composition comprising the same amount of a control ABPC.

Embodiment 31 is the pharmaceutical composition of embodiment 30, wherein the composition provides for at least a 20% increase in endolysosomal delivery in the target mammalian cell as compared to a composition comprising the same amount of a control ABPC.

Embodiment 32 is the pharmaceutical composition of embodiment 31, wherein the composition provides for at least a 50% increase in endolysosomal delivery in the target mammalian cell as compared to a composition comprising the same amount of a control ABPC.

Embodiment 33 is the pharmaceutical composition of embodiment 30, wherein the composition provides for at least a 2-fold increase in endolysosomal delivery in the target mammalian cell as compared to a composition comprising the same amount of a control ABPC.

Embodiment 34 is the pharmaceutical composition of embodiment 33, wherein the composition provides for at least a 5-fold increase in endolysosomal delivery in the target mammalian cell as compared to a composition comprising the same amount of a control ABPC.

Embodiment 35 is the pharmaceutical composition of any one of embodiments 1-34, wherein the composition results in a less of a reduction in the level of DLL3 presented on the surface of the target mammalian cell as compared to a composition comprising the same amount of a control ABPC.

Embodiment 36 is the pharmaceutical composition of any one of embodiments 1-34, wherein the composition does not result in a detectable reduction in the level of the DLL3 presented on the surface of the target mammalian cell.

Embodiment 37 is a pharmaceutical composition comprising an effective amount of an antigen-binding protein construct (ABPC) comprising: a first antigen-binding domain that is capable of specifically binding DLL3 or an epitope of DLL3 presented on the surface of a target mammalian cell; and a conjugated toxin, radioisotope, drug, or small molecule, wherein: (a) the dissociation rate of the first antigen-binding domain at a pH of about 4.0 to about 6.5 is faster than the dissociation rate at a pH of about 7.0 to about 8.0; or the dissociation constant (KD) of the first antigen-binding domain at a pH of about 4.0 to about 6.5 is greater than the KD at a pH of about 7.0 to about 8.0; and (b) the composition provides for one or more of: an increase in toxin liberation in the target mammalian cell as compared to a composition comprising the same amount of a control ABPC; an increase in target mammalian cell killing as compared to a composition comprising the same amount of a control ABPC; and an increase in endolysosomal delivery in the target mammalian cell as compared to a composition comprising the same amount of a control ABPC.

Embodiment 38 is the pharmaceutical composition of embodiment 37, wherein the first antigen-binding domain comprises a heavy chain variable domain of rovalpituzumab with one or more amino acids substituted with a histidine.

Embodiment 39 is the pharmaceutical composition of embodiment 37, wherein the first antigen-binding domain comprises a light chain variable domain of rovalpituzumab with one or more amino acids substituted with a histidine.

Embodiment 40 is the pharmaceutical composition of embodiment 37, wherein the first antigen-binding domain comprises: a heavy chain variable domain of rovalpituzumab with one or more amino acids substituted with a histidine; and a light chain variable domain of rovalpituzumab with one or more amino acids substituted with a histidine.

Embodiment 41 is the pharmaceutical composition of embodiment 38 or 40, wherein the heavy chain variable domain of rovalpituzumab comprises SEQ ID NO: 1.

Embodiment 42 is the pharmaceutical composition of embodiment 39 or 40, wherein the light chain variable domain of rovalpituzumab comprises SEQ ID NO: 2.

Embodiment 43 is the pharmaceutical composition of embodiment 37, wherein the first antigen-binding domain comprises a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine.

Embodiment 44 is the pharmaceutical composition of embodiment 37, wherein the first DLL3-binding domain comprises a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 45 is the pharmaceutical composition of embodiment 37, wherein the first DLL3-binding domain comprises: a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine; and a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 46 is the pharmaceutical composition of embodiment 37, 38, or 43, wherein the first antigen-binding domain comprises a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106.

Embodiment 47 is the pharmaceutical composition of embodiment 37, 39, or 44, wherein the first antigen-binding domain comprises a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 48 is the pharmaceutical composition of embodiment 37, 38, or 43, wherein the first antigen-binding domain comprises a heavy chain variable domain that is at least 90% identical to SEQ ID NO: 1, wherein the heavy chain variable domain includes a histidine at two or more positions in SEQ ID NO: 1 selected from the group consisting of: 29, 31, 54, 55, 105.

Embodiment 49 is the pharmaceutical composition of embodiment 37, 40, or 45, wherein the first antigen-binding domain comprises: a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 50 is the pharmaceutical composition of embodiment 37, wherein the first antigen-binding domain comprises a heavy chain variable domain of SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, or SEQ ID NO: 52.

Embodiment 51 is the pharmaceutical composition of embodiment 37 or 50, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 52 is the pharmaceutical composition of embodiment 37, wherein the first antigen-binding domain comprises a heavy chain variable domain of SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, and SEQ ID NO: 91.

Embodiment 53 is the pharmaceutical composition of embodiment 52, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 54 is the pharmaceutical composition of any one of embodiments 37-53, wherein the composition provides for an increase in toxin liberation in the target mammalian cell as compared to a composition comprising the same amount of a control ABPC.

Embodiment 55 is the pharmaceutical composition of embodiment 54, wherein the composition provides for at least a 20% increase in toxin liberation in the target mammalian cell as compared to a composition comprising the same amount of a control ABPC.

Embodiment 56 is the pharmaceutical composition of embodiment 55, wherein the composition provides for at least a 50% increase in toxin liberation in the target mammalian cell as compared to a composition comprising the same amount of a control ABPC.

Embodiment 57 is the pharmaceutical composition of embodiment 54, wherein the composition provides for at least a 2-fold increase in toxin liberation in the target mammalian cell as compared to a composition comprising the same amount of a control ABPC.

Embodiment 58 is the pharmaceutical composition of embodiment 57, wherein the composition provides for at least a 5-fold increase in toxin liberation in the target mammalian cell as compared to a composition comprising the same amount of a control ABPC.

Embodiment 59 is the pharmaceutical composition of any one of embodiments 37-58, wherein the composition provides for an increase in target mammalian cell killing as compared to a composition comprising the same amount of a control ABPC.

Embodiment 60 is the pharmaceutical composition of embodiment 59, wherein the composition provides for at least a 20% increase in target mammalian cell killing as compared to a composition comprising the same amount of a control ABPC.

Embodiment 61 is the pharmaceutical composition of embodiment 60, wherein the composition provides for at least a 50% increase in target mammalian cell killing as compared to a composition comprising the same amount of a control ABPC.

Embodiment 62 is the pharmaceutical composition of embodiment 59, wherein the composition provides for at least a 2-fold increase in target mammalian cell killing as compared to a composition comprising the same amount of a control ABPC.

Embodiment 63 is the pharmaceutical composition of embodiment 62, wherein the composition provides for at least a 5-fold increase in target mammalian cell killing as compared to a composition comprising the same amount of a control ABPC.

Embodiment 64 is the pharmaceutical composition of any one of embodiments 37-63, wherein the composition provides for an increase in endolysosomal delivery in the target mammalian cell as compared to a composition comprising the same amount of a control ABPC.

Embodiment 65 is the pharmaceutical composition of embodiment 64, wherein the composition provides for at least a 20% increase in endolysosomal delivery in the target mammalian cell as compared to a composition comprising the same amount of a control ABPC.

Embodiment 66 is the pharmaceutical composition of embodiment 65, wherein the composition provides for at least a 50% increase in endolysosomal delivery in the target mammalian cell as compared to a composition comprising the same amount of a control ABPC.

Embodiment 67 is the pharmaceutical composition of embodiment 64, wherein the composition provides for at least a 2-fold increase in endolysosomal delivery in the target mammalian cell as compared to a composition comprising the same amount of a control ABPC.

Embodiment 68 is the pharmaceutical composition of embodiment 67, wherein the composition provides for at least a 5-fold increase in endolysosomal delivery in the target mammalian cell as compared to a composition comprising the same amount of a control ABPC.

Embodiment 69 is the pharmaceutical composition of any one of embodiments 37-68, wherein the composition results in a less of a reduction in the level of DLL3 presented on the surface of the target mammalian cell as compared to a composition comprising the same amount of a control ABPC.

Embodiment 70 is the pharmaceutical composition of any one of embodiments 37-68, wherein the composition does not result in a detectable reduction in the level of the DLL3 presented on the surface of the target mammalian cell.

Embodiment 71 is the pharmaceutical composition of any one of embodiments 1-70, wherein the target mammalian cell is a cancer cell.

Embodiment 72 is the pharmaceutical composition of any one of embodiments 1-71, wherein the dissociation rate of the antigen-binding domain at a pH of about 4.0 to about 6.5 is at least 10% faster than the dissociation rate of the antigen-binding domain at a pH of about 7.0 to about 8.0.

Embodiment 73 is the pharmaceutical composition of any one of embodiments 1-71, wherein the dissociation rate of the antigen-binding domain at a pH of about 4.0 to about 6.5 is at least 3-fold faster than the dissociation rate of the antigen-binding domain at a pH of about 7.0 to about 8.0.

Embodiment 74 is the pharmaceutical composition of any one of embodiments 1-71, wherein the dissociation rate of the antigen-binding domain at a pH of about 4.0 to about 6.5 is at least 10-fold faster than the dissociation rate of the antigen-binding domain at a pH of about 7.0 to about 8.0.

Embodiment 75 is the pharmaceutical composition of any one of embodiments 1-74, wherein the KD of the antigen-binding domain at a pH of about 4.0 to about 6.5 is at least 10% greater than the KD of the antigen-binding domain at a pH of about 7.0 to about 8.0.

Embodiment 76 is the pharmaceutical composition of any one of embodiments 1-74, wherein the KD of the antigen-binding domain at a pH of about 4.0 to about 6.5 is at least 3-fold greater than the KD of the antigen-binding domain at a pH of about 7.0 to about 8.0.

Embodiment 77 is the pharmaceutical composition of any one of embodiments 1-74, wherein the KD of the antigen-binding domain at a pH of about 4.0 to about 6.5 is at least 10-fold greater than the KD of the antigen-binding domain at a pH of about 7.0 to about 8.0.

Embodiment 78 is the pharmaceutical composition of any one of embodiments 1-77, wherein the ABPC is cytotoxic or cytostatic to the target mammalian cell.

Embodiment 79 is the pharmaceutical composition of any one of embodiments 1-78, wherein the ABPC is cross-reactive with a non-human primate DLL3 and human DLL3.

Embodiment 80 is the pharmaceutical composition of any one of embodiments 1-78, wherein the ABPC is cross-reactive with a non-human primate DLL3, a human DLL3, and one or both of rat DLL3 and a mouse DLL3.

Embodiment 81 is the pharmaceutical composition of embodiment 80, wherein the ABPC is cross-reactive with a non-human primate DLL3, a human DLL3, a rat DLL3, and a mouse DLL3.

Embodiment 82 is the pharmaceutical composition of any one of embodiments 1-81, wherein the antigen-binding domain binds to an epitope of DLL3 that is present on the surface of cells from an Old World Monkey.

Embodiment 83 is the pharmaceutical composition of any one of embodiments 1-82, wherein the ABPC comprises a single polypeptide.

Embodiment 84 is the pharmaceutical composition of embodiment 83, wherein the antigen-binding domain is selected from the group consisting of. a VH domain, a VHH domain, a VNAR domain, and a scFv.

Embodiment 85 is the pharmaceutical composition of embodiment 83 or 84, wherein the ABPC is a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv.

Embodiment 86 is the pharmaceutical composition of any one of embodiments 1-82, wherein the ABPC comprises two or more polypeptides.

Embodiment 87 is the pharmaceutical composition of embodiment 86, wherein the ABPC is selected from the group of an antibody, a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate.

Embodiment 88 is the pharmaceutical composition of any one of embodiments 19-87, wherein at least one polypeptide of the ABPC is conjugated to the toxin, the radioisotope, the drug, or the small molecule via a cleavable linker.

Embodiment 89 is the pharmaceutical composition of any one of embodiments 19-87, wherein at least one polypeptide of the ABPC is conjugated to the toxin, the radioisotope, the drug, or the small molecule via a non-cleavable linker.

Embodiment 90 is the pharmaceutical composition of any of embodiments 1-89, wherein the half-life of the ABPC in vivo is decreased as compared to the half-life of a control ABPC in vivo.

Embodiment 91 is the pharmaceutical composition of embodiment 90, wherein the half-life of the ABPC in vivo is decreased about 5% to about 95% as compared to the half-life of a control ABPC in vivo.

Embodiment 92 is the pharmaceutical composition of embodiment 90, wherein the half-life of the ABPC in vivo is decreased about 10% to about 95% as compared to the half-life of a control ABPC in vivo.

Embodiment 93 is the pharmaceutical composition of embodiment 90, wherein the half-life of the ABPC in vivo is decreased about 30% to about 95% as compared to the half-life of a control ABPC in vivo.

Embodiment 94 is the pharmaceutical composition of embodiment 90, wherein the half-life of the ABPC in vivo is decreased about 50% to about 95% as compared to the half-life of a control ABPC in vivo.

Embodiment 95 is the pharmaceutical composition of embodiment 90, wherein the half-life of the ABPC in vivo is decreased about 70% to about 95% as compared to the half-life of a control ABPC in vivo.

Embodiment 96 is the pharmaceutical composition of any one of embodiments 20-95, wherein the control ABPC is capable of specifically binding to DLL3 or an epitope of DLL3 presented on the surface of a target mammalian cell, wherein: (a) the control ABPC comprises a first antigen-binding domain: (b) the dissociation rate of the first antigen-binding domain of the control ABPC at a pH of about 4.0 to about 6.5 is no more than 3-fold faster than the dissociation rate at a pH of about 7.0 to about 8.0; and (c) the dissociation constant (KD) of the first antigen-binding domain of the control ABPC at a pH of about 4.0 to about 6.5 is no more than 3-fold greater than the KD at a pH of about 7.0 to about 8.0.

Embodiment 97 is the pharmaceutical composition of any one of embodiments 20-95, wherein the control ABPC is capable of specifically binding to DLL3 or an epitope of DLL3 presented on the surface of a target mammalian cell, wherein: (a) the control ABPC comprises a first antigen-binding domain; (b) the dissociation rate of the first antigen-binding domain of the control ABPC at a pH of about 4.0 to about 6.5 is no more than 2-fold faster than the dissociation rate at a pH of about 7.0 to about 8.0; and (c) the dissociation constant (KD) of the first antigen-binding domain of the control ABPC at a pH of about 4.0 to about 6.5 is no more than 2-fold greater than the KD at a pH of about 7.0 to about 8.0.

Embodiment 98 is the pharmaceutical composition of any one of embodiments 20-95, wherein the control ABPC is capable of specifically binding to DLL3 or an epitope of DLL3 presented on the surface of a target mammalian cell, wherein: (a) the control ABPC comprises a first antigen-binding domain; (b) the dissociation rate of the first antigen-binding domain of the control ABPC at a pH of about 4.0 to about 6.5 is no more than 1-fold faster than the dissociation rate at a pH of about 7.0 to about 8.0; and (c) the dissociation constant (KD) of the first antigen-binding domain of the control ABPC at a pH of about 4.0 to about 6.5 is no more than 1-fold greater than the KD at a pH of about 7.0 to about 8.0.

Embodiment 99 is the pharmaceutical composition of any one of embodiments 20-95, wherein the control ABPC is rovalpituzumab.

Embodiment 100 is the pharmaceutical composition of any one of embodiments 1-99, wherein the ABPC further comprises a second antigen-binding domain.

Embodiment 101 is a kit comprising at least one dose of the pharmaceutical composition of any one of embodiments 1-100.

Embodiment 102 is an antigen-binding protein construct (ABPC) comprising: a first antigen-binding domain that is capable of specifically binding DLL3 or an epitope of DLL3 presented on the surface of a target mammalian cell, wherein: (a) the dissociation rate of the first antigen-binding domain at a pH of about 4.0 to about 6.5 is faster than the dissociation rate at a pH of about 7.0 to about 8.0; or (b) the dissociation constant (KD) of the first antigen-binding domain at a pH of about 4.0 to about 6.5 is greater than the KD at a pH of about 7.0 to about 8.0.

Embodiment 103 is the ABPC of embodiment 102, wherein the first antigen-binding domain comprises a heavy chain variable domain of rovalpituzumab with one or more amino acids substituted with a histidine.

Embodiment 104 is the ABPC of embodiment 102, wherein the first antigen-binding domain comprises a light chain variable domain of rovalpituzumab with one or more amino acids substituted with a histidine.

Embodiment 105 is the ABPC of embodiment 102, wherein the first antigen-binding domain comprises: a heavy chain variable domain of rovalpituzumab with one or more amino acids substituted with a histidine; and a light chain variable domain of rovalpituzumab with one or more amino acids substituted with a histidine.

Embodiment 106 is the ABPC of embodiment 103 or 105, wherein the heavy chain variable domain of rovalpituzumab comprises SEQ ID NO: 1.

Embodiment 107 is the ABPC of embodiment 104 or 105, wherein the light chain variable domain of rovalpituzumab comprises SEQ ID NO: 2.

Embodiment 108 is the ABPC of embodiment 102, wherein the first antigen-binding domain comprises a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine.

Embodiment 109 is the ABPC of embodiment 102, wherein the first DLL3-binding domain comprises a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 110 is the ABPC of embodiment 102, wherein the first DLL3-binding domain comprises: a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine; and a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 111 is the ABPC of embodiment 102, 103, or 108, wherein the first antigen-binding domain comprises a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106.

Embodiment 112 is the ABPC of embodiment 102, 104, or 109, wherein the first antigen-binding domain comprises a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 113 is the ABPC of embodiment 102, 103, or 108, wherein the first antigen-binding domain comprises a heavy chain variable domain that is at least 90% identical to SEQ ID NO: 1, wherein the heavy chain variable domain includes a histidine at two or more positions in SEQ ID NO: 1 selected from the group consisting of: 29, 31, 54, 55, 105.

Embodiment 114 is the ABPC of embodiment 102, 105, or 110, wherein the first antigen-binding domain comprises: a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 115 is the ABPC of embodiment 102, wherein the first antigen-binding domain comprises a heavy chain variable domain of SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, or SEQ ID NO: 52.

Embodiment 116 is the ABPC of embodiment 102 or 115, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 117 is the ABPC of embodiment 102, wherein the first antigen-binding domain comprises a heavy chain variable domain of SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91.

Embodiment 118 is the ABPC of embodiment 117, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 119 is the ABPC of any one of embodiments 102-118, wherein the ABPC is degraded in the target mammalian cell following internalization of the ABPC by the target mammalian cell.

Embodiment 120 is the ABPC of any one of embodiments 102-119, wherein the ABPC further comprises a conjugated toxin, radioisotope, drug, or small molecule.

Embodiment 121 is the ABPC of embodiment 120, wherein a composition comprising the ABPC provides for an increase in toxin liberation in the target mammalian cell as compared to a composition comprising the same amount of a control ABPC.

Embodiment 122 is the ABPC of embodiment 121, wherein a composition comprising the ABPC provides for at least a 20% increase in toxin liberation in the target mammalian cell as compared to a composition comprising the same amount of a control ABPC.

Embodiment 123 is the ABPC of embodiment 122, wherein a composition comprising the ABPC provides for at least a 50% increase in toxin liberation in the target mammalian cell as compared to a composition comprising the same amount of a control ABPC.

Embodiment 124 is the ABPC of embodiment 121, wherein a composition comprising the ABPC provides for at least a 2-fold increase in toxin liberation in the target mammalian cell as compared to a composition comprising the same amount of a control ABPC.

Embodiment 125 is the ABPC of embodiment 124, wherein a composition comprising the ABPC provides for at least a 5-fold increase in toxin liberation in the target mammalian cell as compared to a composition comprising the same amount of a control ABPC.

Embodiment 126 is the ABPC of any one of embodiments 120-125, wherein a composition comprising the ABPC provides for an increase in target mammalian cell killing as compared to a composition comprising the same amount of a control ABPC.

Embodiment 127 is the ABPC of embodiment 126, wherein a composition comprising the ABPC provides for at least a 20% increase in target mammalian cell killing as compared to a composition comprising the same amount of a control ABPC.

Embodiment 128 is the ABPC of embodiment 127, wherein a composition comprising the ABPC provides for at least a 50% increase in target mammalian cell killing as compared to a composition comprising the same amount of a control ABPC.

Embodiment 129 is the ABPC of embodiment 126, wherein a composition comprising the ABPC provides for at least a 2-fold increase in target mammalian cell killing as compared to a composition comprising the same amount of a control ABPC.

Embodiment 130 is the ABPC of embodiment 129, wherein a composition comprising the ABPC provides for at least a 5-fold increase in target mammalian cell killing as compared to a composition comprising the same amount of a control ABPC.

Embodiment 131 is the ABPC of any one of embodiments 102-130, wherein a composition comprising the ABPC provides for an increase in endolysosomal delivery in the target mammalian cell as compared to a composition comprising the same amount of a control ABPC.

Embodiment 132 is the ABPC of embodiment 131, wherein a composition comprising the ABPC provides for at least a 20% increase in endolysosomal delivery in the target mammalian cell as compared to a composition comprising the same amount of a control ABPC.

Embodiment 133 is the ABPC of embodiment 132, wherein a composition comprising the ABPC provides for at least a 50% increase in endolysosomal delivery in the target mammalian cell as compared to a composition comprising the same amount of a control ABPC.

Embodiment 134 is the ABPC of embodiment 131, wherein a composition comprising the ABPC provides for at least a 2-fold increase in endolysosomal delivery in the target mammalian cell as compared to a composition comprising the same amount of a control ABPC.

Embodiment 135 is the ABPC of embodiment 134, wherein a composition comprising the ABPC provides for at least a 5-fold increase in endolysosomal delivery in the target mammalian cell as compared to a composition comprising the same amount of a control ABPC.

Embodiment 136 is the ABPC of any one of embodiments 102-135, wherein a composition comprising the ABPC results in a less of a reduction in the level of DLL3 presented on the surface of the target mammalian cell as compared to a composition comprising the same amount of a control ABPC.

Embodiment 137 is the ABPC of any one of embodiments 102-135, wherein a composition comprising the ABPC does not result in a detectable reduction in the level of the DLL3 presented on the surface of the target mammalian cell.

Embodiment 138 is an antigen-binding protein construct (ABPC) comprising: a first antigen-binding domain that is capable of specifically binding DLL3 or an epitope of DLL3 presented on the surface of a target mammalian cell; and a conjugated toxin, radioisotope, drug, or small molecule, wherein: (a) the dissociation rate of the first antigen-binding domain at a pH of about 4.0 to about 6.5 is faster than the dissociation rate at a pH of about 7.0 to about 8.0; or the dissociation constant (KD) of the first antigen-binding domain at a pH of about 4.0 to about 6.5 is greater than the KD at a pH of about 7.0 to about 8.0; and (b) the composition provides for one or more of: an increase in toxin liberation in the target mammalian cell as compared to a composition comprising the same amount of a control ABPC; an increase in target mammalian cell killing as compared to a composition comprising the same amount of a control ABPC; and an increase in endolysosomal delivery in the target mammalian cell as compared to a composition comprising the same amount of a control ABPC.

Embodiment 139 is the ABPC of embodiment 138, wherein the first antigen-binding domain comprises a heavy chain variable domain of rovalpituzumab with one or more amino acids substituted with a histidine.

Embodiment 140 is the ABPC of embodiment 138, wherein the first antigen-binding domain comprises a light chain variable domain of rovalpituzumab with one or more amino acids substituted with a histidine.

Embodiment 141 is the ABPC of embodiment 138, wherein the first antigen-binding domain comprises: a heavy chain variable domain of rovalpituzumab with one or more amino acids substituted with a histidine; and a light chain variable domain of rovalpituzumab with one or more amino acids substituted with a histidine.

Embodiment 142 is the ABPC of embodiment 139 or 141, wherein the heavy chain variable domain of rovalpituzumab comprises SEQ ID NO: 1.

Embodiment 143 is the ABPC of embodiment 140 or 141, wherein the light chain variable domain of rovalpituzumab comprises SEQ ID NO: 2.

Embodiment 144 is the ABPC of embodiment 138, wherein the first antigen-binding domain comprises a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine.

Embodiment 145 is the ABPC of embodiment 138, wherein the first DLL3-binding domain comprises a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 146 is the ABPC of embodiment 138, wherein the first DLL3-binding domain comprises: a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine; and a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine.

Embodiment 147 is the ABPC of embodiment 138, 139, or 144, wherein the first antigen-binding domain comprises a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106.

Embodiment 148 is the ABPC of embodiment 138, 140, or 145, wherein the first antigen-binding domain comprises a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 149 is the ABPC of embodiment 138, 139, or 144, wherein the first antigen-binding domain comprises a heavy chain variable domain that is at least 90% identical to SEQ ID NO: 1, wherein the heavy chain variable domain includes a histidine at two more positions in SEQ ID NO: 1 selected from the group consisting of: 29, 31, 54, 55, and 105.

Embodiment 150 is the ABPC of embodiment 138, 141, or 146, wherein the first antigen-binding domain comprises: a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96.

Embodiment 151 is the ABPC of embodiment 138, wherein the first antigen-binding domain comprises a heavy chain variable domain of SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, or SEQ ID NO: 52.

Embodiment 152 is the ABPC of embodiment 138 or 151, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 153 is the ABPC of embodiment 138, wherein the first antigen-binding domain comprises a heavy chain variable domain of SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.

Embodiment 154 is the ABPC of embodiment 153, wherein the first antigen-binding domain comprises a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 66, SEQ ID NO: 69, SEQ ID NO: 72, SEQ ID NO: 73, SEQ ID NO: 76, SEQ ID NO: 77, SEQ ID NO: 78, and SEQ ID NO: 79.

Embodiment 155 is the ABPC of any one of embodiments 138-154, wherein a composition comprising the ABPC provides for an increase in toxin liberation in the target mammalian cell as compared to a composition comprising the same amount of a control ABPC.

Embodiment 156 is the ABPC of embodiment 155, wherein a composition comprising the ABPC provides for at least a 20% increase in toxin liberation in the target mammalian cell as compared to a composition comprising the same amount of a control ABPC.

Embodiment 157 is the ABPC of embodiment 156, wherein a composition comprising the ABPC provides for at least a 50% increase in toxin liberation in the target mammalian cell as compared to a composition comprising the same amount of a control ABPC.

Embodiment 158 is the ABPC of embodiment 155, wherein a composition comprising the ABPC provides for at least a 2-fold increase in toxin liberation in the target mammalian cell as compared to a composition comprising the same amount of a control ABPC.

Embodiment 159 is the ABPC of embodiment 158, wherein a composition comprising the ABPC provides for at least a 5-fold increase in toxin liberation in the target mammalian cell as compared to a composition comprising the same amount of a control ABPC.

Embodiment 160 is the ABPC of any one of embodiments 138-159, wherein a composition comprising the ABPC provides for an increase in target mammalian cell killing as compared to a composition comprising the same amount of a control ABPC.

Embodiment 161 is the ABPC of embodiment 160, wherein a composition comprising the ABPC provides for at least a 20% increase in target mammalian cell killing as compared to a composition comprising the same amount of a control ABPC.

Embodiment 162 is the ABPC of embodiment 161, wherein a composition comprising the ABPC provides for at least a 50% increase in target mammalian cell killing as compared to a composition comprising the same amount of a control ABPC.

Embodiment 163 is the ABPC of embodiment 160, wherein a composition comprising the ABPC provides for at least a 2-fold increase in target mammalian cell killing as compared to a composition comprising the same amount of a control ABPC.

Embodiment 164 is the ABPC of embodiment 163, wherein a composition comprising the ABPC provides for at least a 5-fold increase in target mammalian cell killing as compared to a composition comprising the same amount of a control ABPC.

Embodiment 165 is the ABPC of any one of embodiments 138-164, wherein a composition comprising the ABPC provides for an increase in endolysosomal delivery in the target mammalian cell as compared to a composition comprising the same amount of a control ABPC.

Embodiment 166 is the ABPC of embodiment 165, wherein a composition comprising the ABPC provides for at least a 20% increase in endolysosomal delivery in the target mammalian cell as compared to a composition comprising the same amount of a control ABPC.

Embodiment 167 is the ABPC of embodiment 166, wherein a composition comprising the ABPC provides for at least a 50% increase in endolysosomal delivery in the target mammalian cell as compared to a composition comprising the same amount of a control ABPC.

Embodiment 168 is the ABPC of embodiment 165, wherein a composition comprising the ABPC provides for at least a 2-fold increase in endolysosomal delivery in the target mammalian cell as compared to a composition comprising the same amount of a control ABPC.

Embodiment 169 is the ABPC of embodiment 168, wherein a composition comprising the ABPC provides for at least a 5-fold increase in endolysosomal delivery in the target mammalian cell as compared to a composition comprising the same amount of a control ABPC.

Embodiment 170 is the ABPC of any one of embodiments 138-169, wherein a composition comprising the ABPC results in a less of a reduction in the level of DLL3 presented on the surface of the target mammalian cell as compared to a composition comprising the same amount of a control ABPC.

Embodiment 171 is the ABPC of any one of embodiments 138-169, wherein a composition comprising the ABPC does not result in a detectable reduction in the level of the DLL3 presented on the surface of the target mammalian cell.

Embodiment 172 is the ABPC of any one of embodiments 102-171, wherein the target mammalian cell is a cancer cell.

Embodiment 173 is the ABPC of any one of embodiments 102-172, wherein the dissociation rate of the antigen-binding domain at a pH of about 4.0 to about 6.5 is at least 10% faster than the dissociation rate of the antigen-binding domain at a pH of about 7.0 to about 8.0.

Embodiment 174 is the ABPC of any one of embodiments 102-172, wherein the dissociation rate of the antigen-binding domain at a pH of about 4.0 to about 6.5 is at least 3-fold faster than the dissociation rate of the antigen-binding domain at a pH of about 7.0 to about 8.0.

Embodiment 175 is the ABPC of any one of embodiments 102-172, wherein the dissociation rate of the antigen-binding domain at a pH of about 4.0 to about 6.5 is at least 10-fold faster than the dissociation rate of the antigen-binding domain at a pH of about 7.0 to about 8.0.

Embodiment 176 is the ABPC of any one of embodiments 102-175, wherein the KD of the antigen-binding domain at a pH of about 4.0 to about 6.5 is at least 10% greater than the KD of the antigen-binding domain at a pH of about 7.0 to about 8.0.

Embodiment 177 is the ABPC of any one of embodiments 102-175, wherein the KD of the antigen-binding domain at a pH of about 4.0 to about 6.5 is at least 3-fold greater than the KD of the antigen-binding domain at a pH of about 7.0 to about 8.0.

Embodiment 178 is the ABPC of any one of embodiments 102-175, wherein the KD of the antigen-binding domain at a pH of about 4.0 to about 6.5 is at least 10-fold greater than the KD of the antigen-binding domain at a pH of about 7.0 to about 8.0.

Embodiment 179 is the ABPC of any one of embodiments 102-178, wherein the ABPC is cytotoxic or cytostatic to the target mammalian cell.

Embodiment 180 is the ABPC of any one of embodiments 102-179, wherein the ABPC is cross-reactive with a non-human primate DLL3 and human DLL3.

Embodiment 181 is the pharmaceutical composition of any one of embodiments 102-179, wherein the ABPC is cross-reactive with a non-human primate DLL3, a human DLL3, and one or both of rat DLL3 and a mouse DLL3.

Embodiment 182 is the pharmaceutical composition of embodiment 181, wherein the ABPC is cross-reactive with a non-human primate DLL3, a human DLL3, a rat DLL3, and a mouse DLL3.

Embodiment 183 is the ABPC of any one of embodiments 102-182, wherein the antigen-binding domain binds to an epitope of DLL3 that is present on the surface of cells from an Old World Monkey.

Embodiment 184 is the ABPC of any one of embodiments 102-183, wherein the ABPC comprises a single polypeptide.

Embodiment 185 is the ABPC of embodiment 184, wherein the antigen-binding domain is selected from the group consisting of: a VH domain, a VHH domain, a VNAR domain, and a scFv.

Embodiment 186 is the ABPC of embodiment 184 or 185, wherein the ABPC is a BiTe, a (scFv)2, a nanobody, a nanobody-HSA, a DART, a TandAb, a scDiabody, a scDiabody-CH3, scFv-CH-CL-scFv, a HSAbody, scDiabody-HSA, or a tandem-scFv.

Embodiment 187 is the ABPC of any one of embodiments 102-183, wherein the ABPC comprises two or more polypeptides.

Embodiment 188 is the ABPC of embodiment 187, wherein the ABPC is selected from the group of an antibody, a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate.

Embodiment 189 is the ABPC of any one of embodiments 120-188, wherein at least one polypeptide of the ABPC is conjugated to the toxin, the radioisotope, the drug, or the small molecule via a cleavable linker.

Embodiment 190 is the ABPC of any one of embodiments 120-188, wherein at least one polypeptide of the ABPC is conjugated to the toxin, the radioisotope, the drug, or the small molecule via a non-cleavable linker.

Embodiment 191 is the ABPC of any of embodiments 102-190, wherein the half-life of the ABPC in vivo is decreased as compared to the half-life of a control ABPC in vivo.

Embodiment 192 is the ABPC of embodiment 191, wherein the half-life of the ABPC in vivo is decreased about 5% to about 95% as compared to the half-life of a control ABPC in vivo.

Embodiment 193 is the ABPC of embodiment 191, wherein the half-life of the ABPC in vivo is decreased about 10% to about 95% as compared to the half-life of a control ABPC in vivo.

Embodiment 194 is the ABPC of embodiment 191, wherein the half-life of the ABPC in vivo is decreased about 30% to about 95% as compared to the half-life of a control ABPC in vivo.

Embodiment 195 is the ABPC of embodiment 191, wherein the half-life of the ABPC in vivo is decreased about 50% to about 95% as compared to the half-life of a control ABPC in vivo.

Embodiment 196 is the ABPC of embodiment 191, wherein the half-life of the ABPC in vivo is decreased about 70% to about 95% as compared to the half-life of a control ABPC in vivo.

Embodiment 197 is the ABPC of any one of embodiments 121-196, wherein the control ABPC is capable of specifically binding to DLL3 or an epitope of DLL3 presented on the surface of a target mammalian cell, wherein: (a) the control ABPC comprises a first antigen-binding domain; (b) the dissociation rate of the first antigen-binding domain of the control ABPC at a pH of about 4.0 to about 6.5 is no more than 3-fold faster than the dissociation rate at a pH of about 7.0 to about 8.0; and (c) the dissociation constant (KD) of the first antigen-binding domain of the control ABPC at a pH of about 4.0 to about 6.5 is no more than 3-fold greater than the KD at a H of about 7.0 to about 8.0.

Embodiment 198 is the ABPC of any one of embodiments 121-196, wherein the control ABPC is capable of specifically binding to DLL3 or an epitope of DLL3 presented on the surface of a target mammalian cell, wherein: (a) the control ABPC comprises a first antigen-binding domain; (b) the dissociation rate of the first antigen-binding domain of the control ABPC at a pH of about 4.0 to about 6.5 is no more than 2-fold faster than the dissociation rate at a pH of about 7.0 to about 8.0; and (c) the dissociation constant (KD) of the first antigen-binding domain of the ABPC at a pH of about 4.0 to about 6.5 is no more than 2-fold greater than the KD at a pH of about 7.0 to about 8.0.

Embodiment 199 is the ABPC of any one of embodiments 121-196, wherein the control ABPC is capable of specifically binding to DLL3 or an epitope of DLL3 presented on the surface of a target mammalian cell, wherein: (a) the control ABPC comprises a first antigen-binding domain; (b) the dissociation rate of the first antigen-binding domain of the control ABPC at a pH of about 4.0 to about 6.5 is no more than 1-fold faster than the dissociation rate at a pH of about 7.0 to about 8.0; and (c) the dissociation constant (KD) of the first antigen-binding domain of the control ABPC at a pH of about 4.0 to about 6.5 is no more than 1-fold greater than the KD at a pH of about 7.0 to about 8.0.

Embodiment 200 is the ABPC of any one of embodiments 121-196, wherein the control ABPC is rovalpituzumab.

Embodiment 201 is the ABPC of any one of embodiments 102-200, wherein the ABPC further comprises a second antigen-binding domain.

Embodiment 202 is a kit comprising at least one dose of the ABPC of any one of embodiments 102-201.

Embodiment 203 is a method of treating a cancer characterized by having a population of cancer cells that have DLL3 or an epitope of DLL3 presented on their surface, the method comprising: administering a therapeutically effective amount of the pharmaceutical composition of any one of embodiments 1-100 or the ABPC of any one of embodiments 102-201 to a subject identified as having a cancer characterized by having the population of cancer cells.

Embodiment 204 is a method of reducing the volume of a tumor in a subject, wherein the tumor is characterized by having a population of cancer cells that have DLL3 or an epitope of DLL3 presented on their surface, the method comprising: administering a therapeutically effective amount of the pharmaceutical composition of any one of embodiments 1-100 or the ABPC of any one of embodiments 102-201 to a subject identified as having a cancer characterized by having the population of cancer cells.

Embodiment 205 is a method of inducing cell death in a cancer cell in a subject, wherein the cancer cell has DLL3 or an epitope of DLL3 presented on its surface, wherein the method comprises: administering a therapeutically effective amount of the pharmaceutical composition of any one of embodiments 1-98 or the ABPC of any one of embodiments 102-201 to a subject identified as having a cancer characterized by having a population of the cancer cells.

Embodiment 206 is the method of any one of embodiments 203-205, wherein the cancer is a primary tumor.

Embodiment 207 is the method of any one of embodiments 203-205, wherein the cancer is a metastasis.

Embodiment 208 is the method of any one of embodiments 203-207, wherein the cancer is a non-T-cell-infiltrating tumor.

Embodiment 209 is the method of any one of embodiments 203-207, wherein the cancer is a T-cell infiltrating tumor.

Embodiment 210 is a method of decreasing the risk of developing a metastasis or decreasing the risk of developing an additional metastasis in a subject having a cancer, wherein the cancer is characterized by having a population of cancer cells that have DLL3 or an epitope of DLL3 presented on their surface the method comprising: administering a therapeutically effective amount of the pharmaceutical composition of any one of embodiments 1-100 or the ABPC of any one of embodiments 102-201 to a subject identified as having a cancer characterized by having the population of cancer cells.

Embodiment 211 is the method of embodiment 210, wherein the cancer is a non-T-cell-infiltrating tumor.

Embodiment 212 is the method of embodiment 210, wherein the cancer is a T-cell infiltrating tumor.

The invention is further described in the following examples, which do not limit the scope of the invention described in the claims.

### EXAMPLES

### Example 1. Generation of DLL3 Binders and Engineering of pH Binding Dependence

pH-engineered ABPCs specific for DLL3 are generated using two methods. In the first approach, published monoclonal antibodies against DLL3 are used as a starting template for introduction of additional mutations that allow engineering of pH-dependent binding to DLL3 and i) enhanced endolysosomal accumulation of a conjugated toxin, as well as ii) enhanced DLL3 recycling to the cell surface. The second approach involves discovery of de novo ABPCs specific for DLL3 via antibody display methods from naive libraries or libraries with defined CDR compositions and screening under conditions designed for selection of pH-engineered ABPCs specific for DLL3. In either case, histidine residues play an important role in engineering pH-dependent binding proteins.

Histidine residues are at least partially protonated at a pH below 6.5 owing to its pKa of 6.0. Therefore, if a histidine side chain in an antigen-binding domain participates in an electrostatic binding interaction with its antigen it will start to turn positively charged at a pH at or below 6.5. This could either weaken or enhance the binding affinity of the interaction at a pH below 6.5, based on the corresponding charge of and interactions with the antigen epitope. Thus, systematic introduction of histidines into antibody complementarity determining regions (CDRs) in an antibody or other binder library (e.g., an scFv library) can be used to identify substitutions that will affect an antigen-binding domain's interaction with an antigen at lower pH values. The first approach therefore involves histidine-scanning of variable region sequences of published monoclonal antibodies to identify pH-dependent variants.

Multiple DLL3-binding monoclonal antibodies have been described in the literature and can be used as a template for engineering pH-dependent binding [Saunders L R et al (2015) “A DLL3-targeted antibody-drug conjugate eradicates high-grade pulmonary neuroendocrine tumor-initiating cells in vivo.” Science Translational Medicine 7(302):302ra136, Puca L et al (2019) “Delta-like protein 3 expression and therapeutic targeting in neuroendocrine prostate cancer” Science Translational Medicine 484:eaav0891]. Briefly, for a subset of the antibody sequences, CDRs in each chain are identified using the methods described by Kabat et al (Kabat et al. (1992) Sequences of Proteins of Immunological Interest (DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To engineer pH-dependent sequence variants, individual amino acid residues within the heavy chain and/or light chain CDRs are systematically substituted with a histidine, one at a time. In cases where the starting CDR residue is a histidine, it is mutated to an alanine. Antibody variants with only one histidine or alanine mutation in a heavy/light chain CDR are generated by co-transfection of Expi293 cells with a) one heavy chain or light chain sequence variant, and b) the corresponding starting ABPC (e.g., the starting DLL3-binding monoclonal antibody) light chain or heavy chain, respectively, using methods known to the art. After allowing for a period of protein expression, cell culture supernatants are collected, quantified, and the pH dependence of the variant is evaluated using biolayer interferometry (BLI) or other methods known to the art. Briefly, cell culture supernatants are normalized to an antibody expression level of 50 μg/mL, and captured on an anti-human Fc sensor (Forte Bio). A baseline is established using 1× kinetics buffer (Forte Bio), and the sensor is associated with 100 nM of DLL3 in 1×PBS at pH 7.4 for 300 sec to generate an association curve. In the dissociation phase, the antibody-antigen complex on the sensor is exposed to 1×PBS at either pH 5.5 or pH 7.4 for 300-500 sec. Association and dissociation phase curves are examined for the starting ABPC antibody and each corresponding antibody variant at pH 5.5 and pH 7.4 to inform on two criteria: a) enhanced dissociation (i.e., higher k_{off} values) at pH 5.5 due to histidine or alanine substitution compared to the starting ABPC, and b) reduced dissociation at pH 7.4 (i.e., lower k_{off} values) compared to pH 5.5 in the antibody variant itself and with the starting ABPC. Variants that show either enhanced dissociation at pH 5.5 or reduced dissociation at pH 7.4 or both are selected for further analysis. It is also noted that while some histidine and alanine mutations obliterate DLL3 binding, others are tolerated with little (e.g., less than 1-fold change in KD or dissociation rate) or no change in DLL3 binding kinetics. Especially because histidine is a large, positively charged amino acid, these histidine variants and alanine variants with no change are noted as positions that may tolerate a wide range of mutations and lead to antibodies with different sequence but similar binding properties, a designation that is not otherwise apparent. The variants selected for further analysis are expressed at a larger scale and purified using protein A affinity chromatography. Binding kinetics (kₒₙ and k_{off}) of the purified starting ABPC and variant antibodies are measured at pH 5.5 and pH 7.4 using Biacore (GE Healthcare). The ratio of the antibody's rate of dissociation (k_{off} at pH 7.4 divided by k_{off} at pH 5.5) is also used as a quantitative assessment of pH-dependent binding; similarly, the dissociation constant K_{D} is calculated at both pH 5.5 and pH 7.4 as k_{off} divided by kₒₙ and the ratio of the antibody's dissociation constant (K_{D} at pH 7.4 divided by K_{D} at pH 5.5) is also used as a quantitative assessment of pH-dependent binding. Antibodies with a rate of dissociation ratio less than that of the starting ABPC and/or a dissociation constant ratio less than that of the starting ABPC are selected for further assessment of combinatorial substitutions. Favorable histidine and/or alanine amino acid positions can also be combined to enhance pH dependence; this can be done by, e.g., combinatorially or rationally combining histidine and/or alanine substitutions on a given heavy or light chain that individually improve pH dependence, by, e.g., combinatorially or rationally combining modified heavy and light chains such that histidine and/or alanine substitutions are present on both chains, or combinations thereof. Such combinatorial variants are generated and tested/analyzed for differential pH dependence using the methods and protocols described herein, or others known to the art. Antibody variants that have the lowest rate of dissociation ratios and/or dissociation constant ratios are selected as candidates for further analysis (hereafter referred to as “pH-engineered ABPCs specific for DLL3”).

The second method for selection of pH-engineered ABPCs specific for DLL3 involves either screening libraries to identify de novo pH-dependent ABPCs specific for DLL3 or ABPCs that could serve as templates for engineering pH-dependent binding as described herein. Two types of libraries can be used for these selections: naive phage/yeast display antibody libraries (e.g., Fab, scFv, VHH, VL, or others known to the art) or phage/yeast display libraries where CDRs have been mutated to express a subset of amino acid residues. Libraries are screened against soluble recombinant DLL3 extracellular domains using methods known to the art with positive selection for variants that bind weakly (e.g., are eluted from beads) at pH 5.0 and bind strongly (e.g., are bound to beads) at pH 7.4. Three rounds of selections are performed. The final round of binders are screened using ELISA for binding to human DLL3 and cyno DLL3 and mouse DLL3 or via mean fluorescence intensity in flow cytometric analysis. If more binders with cyno or murine cross-reactivity are desired, the final selection round can instead be performed on cyno DLL3 or murine DLL3. Selected binding proteins are subcloned into mammalian expression vectors and expressed as either full IgG proteins or Fc fusions in Expi293 cells. BLI analysis is performed as described herein for selection of pH-dependent binder variants and confirmed using Biacore.

### Example 2. In Vitro Demonstration of pH-Dependent Binding to DLL3, pH-Dependent Release of DLL3, Enhanced Endolysosomal Delivery in DLL3+ Cells, and Increased DLL3 Antigen Density in DLL3+ Cells after Exposure to pH-Engineered ABPCs Specific for DLL3 as Compared to Control ABPCs Specific for DLL3

As discussed herein, pH-engineered ABPCs specific for DLL3 exhibit the desirable property of decreased DLL3 binding at acidic pH (e.g., pH 5.0, pH 5.5), but enhanced binding at higher pH (e.g., pH 7.4), which enhances their accumulation in endolysosomes under physiological conditions.

ph-Dependent Binding to DLL3 on Cells

To demonstrate that pH-engineered ABPCs specific for DLL3 binds cell surface DLL3 at neutral pH, a cell surface binding assay is performed. A panel of human cells that are DLL3+ is assembled (e.g., NCI-H82NCI-H660,U-118-MG). Methods of identifying and quantifying gene expression (e.g., DLL3) for a given cell line are known to the art, and include, e.g., consulting the Cancer Cell Line Encyclopedia (CCLE; https://portals.broadinstitute.org/ccle) to ascertain the expression level and/or mutation status of a given gene in a tumor cell line), rtPCR, microarray, or RNA-Seq analysis, or cell staining with antibodies known in the art (e.g. R&D Systems Human DLL3 Antibody (AB4315) or Abcam Recombinant Anti-DLL3 Antibody, Cat #AB229902 for DLL3). Cells are seeded at approximately 5-10,000 per well in 150 μL of pH 7.4 culture medium and incubated at 37° C. for 5 minutes at several doses (e.g., a two-fold dilution series) from 1 μM to 1 μM with one of the following antibodies: a known, control ABPC specific for DLL3 (e.g., an antibody, rovalpituzumab, SC16.4, SC16.13, SC16.15, SC16.25, SC16.34, SC16.67), the pH-engineered ABPC specific for DLL3, and an appropriate negative isotype control mAb (e.g., Biolegend Purified Human IgG1 Isotype Control Recombinant Antibody, Cat #403501). Prior to the onset of the experiment, the binding properties of all antibodies are validated using methods known to the art. Following the 5 minute incubation, cells are fixed with 4% formaldehyde (20 min at room temperature) and incubated with an appropriate fluorophore-labeled secondary antibody (e.g., ThermoFisher Mouse anti-Human IgG1 Fc Secondary Antibody, Alexa Fluor 488, Cat #A-10631) for 60 minutes. Unbound reagents are washed with a series of PBS washes, and the cell panels are imaged using confocal microscopy. Upon analysis of the images, significant fluorescence can be observed on the surface of cells bound with the known, control ABPC specific for DLL3 as well as the pH-engineered ABPC specific for DLL3, but little surface binding can be observed for the isotype negative control. To isolate the effect of pH on surface binding, the same experiment is repeated twice, with the primary antibody incubation taking place at sequentially lower pH (e.g., pH 6.5 and 5.5 and 5.0). Analysis of the resulting confocal microscopy images can show significant fluorescence on the surface of cells bound with all mAbs tested, excepting the isotype negative control, and that this fluorescence decreases for the pH-engineered ABPC specific for DLL3 as the pH decreases. Alternatively, cells are analyzed for mean fluorescent intensity by flow cytometry using methods known in the art. A dissociation constant KD on cells at neutral pH of the antibodies analyzed is determined by nonlinear regression methods known in the art (e.g., a Scatchard plot). Taken together, the results can show that the pH engineering process results in the creation of a pH-engineered ABPC specific for DLL3 that is pH-dependent in its binding properties and that it more effectively binds at neutral pH as compared to more acidic pH. Other methods of assessing the pH dependence of the pH-engineered ABPCs specific for DLL3 are known in the art and include, e.g., using flow cytometry to measure ABPC surface binding.

pH-Dependent Release of DLL3 on Cells

To demonstrate that pH-engineered ABPCs specific for DLL3 are capable of releasing DLL3 at low pH after binding at a neutral pH, a variant of the cell surface binding assay described above is performed using methods known to the art (e.g., as generally described in Gera N. (2012) PLoS ONE 7(11): e48928). Briefly, an appropriate DLL3+ cell line (passage number less than 25) is harvested and 50,000 cells per well are plated in a U-Bottomed 96-well microplate. Three conditions are tested; binding and secondary staining at pH 7.4, binding and secondary staining at pH 5.0, and binding at pH 7.4 followed by release at pH 5.0 for 30 minutes and secondary staining at pH 7.4. Both pH-engineered ABPCs specific for DLL3 as well as a control ABPC specific for DLL3 are tested. The cells are washed two times with 200 μL of FACS buffer (1×PBS containing 3% Fetal Bovine Serum) at either pH 7.4 or 5.0 depending on the condition being tested. The purified protein samples are diluted into FACS buffer of the appropriate pH and added to the cells and allowed to bind for one hour on ice. After incubation with the primary antibodies the pH 7.4 and pH 5.0 conditions are washed twice as before, and then 100 μl of secondary rat anti-human Fc AF488 (BioLegend 410706) or other appropriate antibody, diluted 1:50, or anti Myc-Tag mouse mAb-AF488 (Cell Signaling Technologies 2279S) diluted 1:50 is added in FACS buffer of the appropriate pH, and incubated for 30 minutes on ice. The pH 5.0 release condition is washed twice with FACS buffer pH 7.4 and then resuspended in 100 μl of FACS buffer pH 5.0 and incubated on ice for 30 minutes, followed by secondary staining in FACS buffer pH 7.4 as described for the other conditions. The plates are washed twice as before and resuspended in 1% paraformaldehyde in the appropriate FACS buffer to fix them for flow cytometry analysis. All conditions are read on a flow cytometer (Accuri C6, BD Biosciences). Binding is observed as a shift in the FL1 signal (as a mean fluorescence intensity) versus secondary alone. Upon analysis of the data, it can be determined that both the pH-engineered ABPC specific for DLL3 as well as the control ABPC specific for DLL3 effectively bind the surface of DLL3+ cells at neutral pH, but the pH-engineered ABPC specific for DLL3 binds poorly at pH 5.0; similarly, it can be determined that the pH-engineered ABPC specific for DLL3 binds effectively at pH 7.4, but then releases/unbinds DLL3 at pH 5.0.

Enhanced Endolysosomal Delivery in DLL3+ Cells of pH-Engineered ABPCs Specific for DLL3 as Compared to Control ABPCs Specific for DLL3 (pHrodo)

To verify and demonstrate that ABPCs specific for DLL3 achieve endolysosomal localization following cellular uptake, an internalization assay is performed using methods known to the art (e.g., Mahmutefendic et al., Int. J. Biochem. Cell Bio., 2011). Briefly, as described herein, a panel of human cells that express DLL3 highly is assembled using methods known to the art. Cells are plated, washed three times with PBS, and incubated at 37 degrees C. for 60 minutes in media at neutral pH, with added concentrations of 2 micrograms per milliliter of a known, control ABPC specific for DLL3 (e.g., as described herein), the pH-engineered ABPC specific for DLL3, and an appropriate negative isotype control mAb (e.g., as described herein). In a subset of cells, validation of antibody internalization and endosomal localization is performed using methods known to the art; e.g., cells are fixed in 4% formaldehyde as described herein, permeabilized using TWEEN 20 or other methods known to the art (Jamur M C et al (2010) Permeabilization of cell membranes, Methods Mol Biol. 588:63-6), additionally stained with an endosomal marker, e.g., a fluorescent RAB11 antibody (RAB11 Antibody, Alexa Fluor 488, 3H18L5, ABfinity™ Rabbit Monoclonal), stained with an appropriate fluorescently labeled anti-human secondary antibody (e.g., as described herein), and imaged using confocal fluorescence microscopy, as described herein. Analysis of the confocal images can be used to show that both the pH-engineered ABPC specific for DLL3 as well as the control ABPC specific for DLL3 are internalized and accumulate in the endolysosomes.

To demonstrate that pH-engineered ABPCs specific for DLL3 achieve enhanced endolysosomal accumulation relative to a control ABPC specific for DLL3, a pHrodo-based internalization assay is performed using both a known, control ABPC specific for DLL3 (e.g., as described herein) as well as the pH-engineered ABPC specific for DLL3. The assay makes use of pHrodo™ iFL (P36014, ThermoFisher), a dye whose fluorescence increases with decreasing pH, such that its level of fluorescence outside the cell at neutral pH is lower than its level of fluorescence inside the acidic pH environment of endolysosomes. Briefly, an appropriate DLL3+ cell line (less than passage 25) is suspended in its recommended media (e.g., by cell banks or cell bank databases ATCC, DSMZ, or ExPASy Cellosaurus) and plated in a 24-well plate at a density of 2,000,000 cells/mL, 1 mL per well. While keeping the cells on ice, 1 mL of 2×pHrodo iFL-labeled antibody (prepared in accordance with the manufacturer's instructions) is added to each well, the well is pipetted/mixed five times, and the plate is incubated in a light-protected environment for 45 minutes, on ice. An identical but separate plate is also incubated on ice that is meant as a no-internalization negative control. Following this incubation, the experimental plate is moved to a 37 degree C. incubator, the negative control plate is kept on ice to slow or block internalization, and samples are taken at designated time points to create an internalization time course. Samples are placed into a U-bottom 96-well plate, and internalization is quenched via addition of 200 μL/well of ice-cold FACS buffer. The plates are spun down at 2000×g for 2 minutes, resuspended in 200 μL ice-cold FACS buffer, spun down again, and resuspended in FACS buffer a second time. Finally, the samples are loaded into a flow cytometer for read-out of cellular pHrodo fluorescence using excitation and emission wavelengths consistent with the excitation and emission maxima of the pHrodo iFL Red dye (566 nm and 590 nm, respectively). Upon completion of the flow cytometry experiment and analysis of the data, it can be observed that cells treated with the pH-engineered ABPC specific for DLL3 have a higher pHrodo iFL signal relative to a known, control ABPC specific for DLL3, indicating that pH-engineered ABPCs specific for DLL3 achieve enhanced endolysosomal accumulation relative to a control ABPC specific for DLL3.

Alternatively, to demonstrate that pH-engineered ABPCs specific for DLL3 achieve enhanced endolysosomal accumulation relative to a control ABPC specific for DLL3, a variation of the above-described experiment is performed. DLL3+ cells are plated, washed three times with PBS, and incubated at 37 degrees C. for 60 minutes in media at neutral pH with added concentrations of 2 μg/mL of either pH-engineered ABPC specific for DLL3 or control ABPC specific for DLL3. Following incubation, cells are washed three times with PBS, fixed and permeabilized, and stained with a panel of appropriately selected antibodies that bind late endosomal markers as well as lysosomes (e.g., RAB7, and LAMP1; Cell Signaling Technology, Endosomal Marker Antibody Sampler Kit #12666; AbCam, Anti-LAMP2 antibody [GL2A7], ab13524). After primary antibody staining, cells are stained with an appropriate mixture of fluorescently labeled secondary antibodies (e.g., Goat Anti-Human IgG (H&L) Secondary Antibody (Alexa Fluor 647)Cat #A-21445, and Abcam Goat Anti-Rabbit IgG H&L (Alexa Fluor 488), Cat #ab150077), imaged using confocal fluorescence microscopy, and regions of co-localization of signal from DLL3-specific antibodies and endosomal markers are visualized and quantified. Upon analysis of the data, it can be revealed that there is increased co-localization of endolysosomal and DLL3-specific antibody signal in wells treated with the pH-engineered ABPCs specific for DLL3 as compared to wells treated with control ABPC specific for DLL3, and can thereby demonstrate that pH-engineered ABPCs specific for DLL3 achieve enhanced endolysosomal accumulation relative to control ABPC specific for DLL3.

Increased DLL3 Antigen Density in DLL3+ Cells after Exposure to pH-Engineered ABPCs Specific for DLL3 as Compared to Control ABPCs Specific for DLL3

To demonstrate that treatment of cells with the pH-engineered ABPCs specific for DLL3 does not result in a detectable reduction of the level of DLL3 on the surface of cells exposed to the pH-engineered ABPCs specific for DLL3, or that said treatment results in less of a reduction of the level of DLL3 on the surface of cells exposed to the pH-engineered ABPC specific for DLL3 versus a control ABPC specific for DLL3, an antigen density study is performed using flow cytometry. Briefly, 4.0×10≡cells that express DLL3 are plated per well in a 96-well plate in 100 μL media. Cells are treated with a titration from 1 μM to 1 μM of i) pH-engineered ABPCs specific for DLL3, ii) a first control ABPC specific for DLL3, iii) an appropriate isotype control, and iv) an untreated control. Cells are incubated for 2 hours at 37° C., at which point all cells are incubated with 200 nM of a fluorophore-labeled second control ABPC specific for DLL3 (e.g., as described herein) which has a different epitope (as determined by, e.g., competitive binding studies on cells) than either the first control ABPC specific for DLL3 or the pH-engineered ABPCs specific for DLL3 for 30 minutes at 4° C. Following this 30-minute incubation, the mean fluorescence intensity (MFI) of all cells is read out using, e.g., flow cytometry, using methods known to one of ordinary skill in the art. In parallel, a quantitative standard curve that can be used to quantify the presence of DLL3 on the surface of treated cells as a function of MFI is generated using a commercially available quantification kit (e.g., BD Biosciences PE Phycoerythrin Fluorescence Quantitation Kit, catalog #340495); the quantitative standard curve is created by following the manufacturer's instructions. Other methods of determining the absolute number of DLL3 on the cell surface are known in the art and include, e.g., use of radioisotopically labeled reagents. Upon analysis of the data, it can be revealed that at least one antibody concentration, cells treated with a control ABPC specific for DLL3 experience a reduction of the level of DLL3 on their surface, whereas cells treated with pH-engineered ABPCs specific for DLL3 experience a significantly smaller reduction or no reduction at all, both relative to the isotype and untreated controls.

### Example 3. Conjugation of pH-Engineered and Control ABPCs Specific for DLL3 to Cytotoxic Drugs

An antigen-binding protein construct conjugate (ADC) is made comprising the DLL3-binding IgG (hereafter, DLL3-IgG) described herein linked to monomethyl auristatin E (MMAE) via a valine-citrulline (ve) linker (hereafter, DLL3-IgG-DC). Conjugation of the antigen-binding protein construct with vcMMAE begins with a partial reduction of the DLL3-IgG followed by reaction with maleimidocaproyl-Val-Cit-PABC-MMAE (vcMMAE). The DLL3-IgG (20 mg/mL) is partially reduced by addition of TCEP (molar equivalents of TCEP:mAb is 2:1) followed by incubation at 0° C. overnight. The reduction reaction is then warmed to 20° C. To conjugate all of the thiols, vcMMAE is added to a final vcMMAE:reduced Cys molar ratio of 1:15. The conjugation reaction is carried out in the presence of 10% v/v of DMSO and allowed to proceed at 20° C. for 60 minutes.

After the conjugation reaction, excess free N(acetyl)-Cysteine (2 equivalents vs. vcMMAE charge) is added to quench unreacted vcMMAE to produce the Cys-Val-Cit-MMAE adduct. The Cys quenching reaction is allowed to proceed at 20° C. for approximately 30 minutes. The Cys-quenched reaction mixture is purified as per below. The above conjugation method can also be used to conjugate maleimidocaproyl monomethylauristatin F (mcMMAF) to an antigen-binding protein construct.

The DLL3-IgG-DC is purified using a batch purification method. The reaction mixture is treated with the appropriate amount of water washed Bu-HIC resin (ToyoPearl; Tosoh Biosciences), i.e., seven weights of resin is added to the mixture. The resin/reaction mixture is stirred for the appropriate time, and monitored by analytical hydrophobic interaction chromatography for removal of drug conjugate products, filtered through a coarse polypropylene filter, and washed by two bed volumes of a buffer (0.28 M sodium chloride, 7 mM potassium phosphate, pH 7). The combined filtrate and rinses are combined and analyzed for product profile by HIC HPLC. The combined filtrate and rinses are buffer exchanged by ultrafiltration/diafiltration (UF/DF) to 15 mM histidine, pH 6 with 10 diavolumes 15 nM histidine buffer.

A similar protocol can be used to conjugate DNA toxins such as SG3249 and SGD-1910 to DLL3-IgG (see Tiberghien A C et al (2016) Design and Synthesis of Tesirine, a Clinical Antibody-Drug Conjugate Pyrrolobenzodiazepine Dimer Payload, ACS Med Chem Lett 7:983-987). Briefly, for SG3249, DLL3-IgG (15 mg, 100 nanomoles) is diluted into 13.5 mL of a reduction buffer containing 10 mM sodium borate pH 8.4, 2.5 mM EDTA and a final antibody concentration of 1.11 mg/mL. A 10 mM solution of TCEP is added (1.5 molar equivalent/antibody, 150 nanomoles, 15 microliters) and the reduction mixture is heated at +37° C. for 1.5 hours in an incubator. After cooling down to room temperature, SG3249 is added as a DMSO solution (5 molar equivalent/antibody, 500 nanomoles, in 1.5 mL DMSO). The solution is mixed for 1.25 hours at room temperature, then the conjugation is quenched by addition of N-acetyl cysteine (1 micromole, 100 microliters at 10 mM), and injected into an AKTA™ Pure FPLC using a GE Healthcare HiLoad™ 26/600 column packed with Superdex 200 PG, and eluted with 2.6 mL/min of sterile-filtered phosphate-buffered saline (PBS). Fractions corresponding to the DLL3-IgG-DC monomer peak are pooled, concentrated using a 15 mL Amicon Ultracell 50 KDa MWCO spin filter, analysed and sterile-filtered. UHPLC analysis on a Shimadzu Prominence system using a Phenomenex Aeris 3.6u XB-C18 150×2.1 mm column eluting with a gradient of water and acetonitrile on a reduced sample of DLL3-IgG-DC at 280 nm and 330 nm (SG3249 specific) can show a mixture of light and heavy chains attached to several molecules of SG3249, consistent with a drug-per-antibody ratio (DAR) of 1 to 4 molecules of SG3249 per antibody. UHPLC analysis on a Shimadzu Prominence system using a Phenomenex Yarra 3u SEC-3000 300 mm×4.60 mm column eluting with sterile-filtered SEC buffer containing 200 mM potassium phosphate pH 6.95, 250 mM potassium chloride and 10% isopropanol (v/v) on a sample of DLL3-IgG-DC at 280 nm can show a monomer purity of over 90% with no impurity detected. UHPLC SEC analysis allows determination of final DLL3-IgG-DC yield of greater than 30%.

Alternatively, methods to conjugate toxins to antibodies via lysine residues are known in the art (e.g., see Catcott K C et al (2016) Microscale screening of antibody libraries as maytansinoid antibody-drug conjugates, MAbs 8:513-23). In addition, similar methods to the above can be used to conjugate drugs and toxins to non-IgG formats with disulfide bonds, such as Vh-Fcs.

### Example 4. Demonstration of Enhanced Cytotoxicity of pH-Engineered ABPC ADCs Specific for DLL3 in DLL3+ Cells as Compared to a Control ABPC ADC Specific for DLL3

The cytotoxic activity of both pH-engineered ADCs specific for DLL3 (e.g., a pH-engineered DLL3-IgG-DC) and control ABPC ADCs specific for DLL3 (e.g., a control ABPC DLL3-IgG-DC) are separately evaluated on a panel of DLL3+ cell lines expressing a variety of antigen densities (e.g., as described herein) and a DLL3− cell line (e.g., A549), selected using the methods described herein, and, optionally, cells expressing transgenic DLL3, e.g., HEK293 cells transfected with DLL3 using methods known in the art (e.g., Expi293™ Expression System Kit ThermoFisher Catalog number: A14635). For purposes of validation, prior to use, all cell lines are tested for expression of DLL3 using methods known to the art, e.g., qPCR, flow cytometry, mRNA RPKM, and antibody staining using anti-DLL3 antibodies known to the art (e.g., as described herein) followed by visualization of the stain using fluorescence microscopy, immunohistochemistry, flow cytometry, ELISA, or other methods known to the art. To evaluate the cytotoxicity of compounds, cells are seeded at approximately 10-40,000 per well in 150 microliters of culture medium, then treated with graded doses of compounds from 1 pM to 1 μM in quadruplicates at the initiation of the assay. Cytotoxicity assays are carried out for 96 hours after addition of test compounds. Fifty microliters of resazurin dye are added to each well during the last 4 to 6 hours of the incubation to assess viable cells at the end of culture. Dye reduction is determined by fluorescence spectrometry using the excitation and emission wavelengths of 535 nm and 590 nm, respectively. For analysis, the extent of resazurin reduction by the treated cells is compared to that of untreated control cells, and percent cytotoxicity is determined. Alternatively, a WST-8 kit is used to measure cytotoxicity per the manufacturer's instructions (e.g., Dojindo Molecular Technologies Catalog #CCK-8). IC50, the concentration at which half-maximal killing is observed, is calculated using curve-fitting methods known in the art. Upon analysis of the data, it can be determined that pH-engineered and control ABPC ADCs specific for DLL3 are substantially cytotoxic to one or more DLL3+ cell line, but less toxic to DLL3-cells. It also can be determined that pH-engineered ADCs specific for DLL3 are more cytotoxic to one or more DLL3+ cell lines than control ABPC ADCs specific for DLL3 because: a) they show greater depth of killing at one or more concentrations or, b) they show lower IC50 or, c) they show a greater ratio of their dissociation constant KD on cells at neutral pH (as described herein) divided by their IC50 on those same cells.

Additionally, the cytotoxic activity of ABPCs specific for DLL3 can be measured in a secondary ADC assay. Secondary ADC assays are known in the art (e.g., Moradec Cat #αHFc-NC-MMAF and Cat #αHFc-CL-MMAE, and associated manufacturer's instructions). Briefly, the assay is carried out as in the previous paragraph, except the ABPC specific for DLL3 is substituted for the ADC specific for DLL3, and to evaluate the cytotoxicity of compounds, cells are seeded at approximately 10-40,000 per well in 150 microliters of culture medium, then treated with graded doses of ABPC specific for DLL3 from 1 pM to 1 μM (final concentration in culture medium, having been pre-mixed with 100 nM, final concentration in culture medium, of Moradec Cat #αHFc-NC-MMAF secondary ADC reagent and pre-incubated at 37° C. for 30 min before addition of the mixture to the culture medium) in quadruplicates at the initiation of the assay.

The cytotoxic activity of pH-engineered ADCs specific for DLL3 and control ABPC ADCs specific for DLL3 conjugates, as well as ABPCs specific for DLL3 in a secondary ADC assay, are additionally measured by a cell proliferation assay employing the following protocol (Promega Corp. Technical Bulletin TB288; Mendoza et al., Cancer Res. 62:5485-5488, 2002):

1. An aliquot of 100 microliters of cell culture containing about 104 cells (e.g., DLL3+ cells as described herein) in medium is deposited in each well of a 96-well, opaque-walled plate.

2. Control wells are prepared containing medium and without cells.

3. ADC specific for DLL3 is added to the experimental wells at a range of concentrations from 1 pM-1 uM and incubated for 1-5 days. Alternatively, in a secondary ADC assay, 100 nM secondary ADC reagent (final concentration in culture medium, Moradec Cat #αHFc-NC-MMAF) and ABPC specific for DLL3 at a range of concentrations from 1 pM-1 uM (final concentration in culture medium) are pre-mixed and pre-incubated at 37° C. for 30 min before addition of the mixture to the culture medium, and incubated for 1-5 days.

4. The plates are equilibrated to room temperature for approximately 30 minutes.

5. A volume of CellTiter-Glo Reagent equal to the volume of cell culture medium present in each well is added.

6. The contents are mixed for 2 minutes on an orbital shaker to induce cell lysis.

7. The plate is incubated at room temperature for 10 minutes to stabilize the luminescence signal.

8. Luminescence is recorded and reported in graphs as RLU=relative luminescence units.

### Example 5. Demonstration of Enhanced Toxin Liberation of pH-Engineered ABPC ADCs Specific for DLL3 in DLL3+ Cells as Compared to a Control ABPC ADC Specific for DLL3

The pH-engineered ADCs specific for DLL3 (e.g., a pH-engineered DLL3-IgG-DC) can also demonstrate increased toxin liberation in DLL3+ cells as compared to a control ABPC ADC specific for DLL3 (e.g., a control ABPC DLL3-IgG-DC). After treatment of DLL3+ cells with pH-engineered and control ABPC ADCs specific for DLL3 as described herein, an LC-MS/MS method is used to quantify unconjugated (i.e., liberated) MMAE in treated DLL3+ cells (Singh, A. P. and Shah, D. K. Drug Metabolism and Disposition 45.11 (2017): 1120-1132.) An LC-MS/MS system with electrospray interphase and triple quadrupole mass spectrometer is used. For the detection of MMAE, a XBridge BEH Amide column (Waters, Milford, Mass.) is used with mobile phase A as water (with 5 mM ammonium formate and 0.1% formic acid) and mobile phase B as 95:5 acetonitrile/water (with 0.1% formic acid and 1 mM ammonium formate), using a gradient at a flow rate of 0.25 mL/min at 40° C. The total duration of the chromatographic run is 12 minutes, where two MRM scans (718.5/686.5 and 718.5/152.1 amu) are monitored. Deuterated (d8) MMAE (MCE MedChem Express, Monmouth Junction, N.J.) is used as an internal standard. First, an equation for quantifying unconjugated MMAE in a biological sample is derived by dividing the peak area for each drug standard by the peak area obtained for the internal standard. The resultant peak area ratios are then plotted as a function of the standard concentrations, and data points are fitted to the curve using linear regression. Three QC samples are included in the low, middle, and upper ranges of the standard curve to assess the predictive capability of the developed standard curve. The standard curves obtained are then used to deduce the observed concentrations of MMAE in a biologic sample. For measurement of MMAE concentration, treated cell samples are pelleted and reconstituted in fresh media to a final concentration of 0.25 million cells/100 μL. Samples are spiked with d8-MMAE (1 ng/mL) before performing cell lysis by the addition of a 2-fold volume of ice-cold methanol followed by freeze-thaw cycle of 45 minutes at −20° C. The final cell lysate is obtained by centrifuging the samples at 13,000 rpm for 15 minutes at 4° C. followed by collection of supernatant. For the preparation of standards and QC samples, a fresh cell suspension (0.25 million/100 μl) is spiked with known concentrations of MMAE and internal standard (d8-MMAE) before a procedure similar to the cell lysis mentioned above. The resulting cell lysates are then evaporated and reconstituted in mobile phase B before injection into LC-MS/MS. The concentration of unconjugated MMAE in lysates of DLL3+ cells treated with pH-engineered ADCs specific for DLL3 is observed to be greater than that in DLL3+ cells treated with control ABPC ADC specific for DLL3.

For tubulin-inhibiting toxins, toxin liberation is also assessed by monitoring of cell viability and cell cycle phase. ˜2.0×10\hat{ }5 DLL3+ cells are plated in a 96-well flat bottom plate and treated with pH-engineered and control ABPC ADCs specific for DLL3 as described herein. After treatment, cells are transferred to a 96-round bottom plate, and the plate is centrifuged at 400 rcf for 2 min to decant supernatant. Decanted cells are stained with Live/Dead eFluor 660. Cells are then centrifuged and washed with FACS buffer (PBS with 2% FBS), after which cell cycle distribution is analyzed with a BD Cycletest™ Plus DNA Kit (cat #340242). Briefly, cells are re-suspended in 76 ul Solution A and incubated for 10 min at room temperature. 61 μL Solution B is then added, and cells are incubated for another 10 min at room temperature. Finally, 61 μL of cold Solution C is added, and cells are again incubated for 10 min at room temp. Immediately after the last incubation step, cells are analyzed by flow cytometry (without washing) at a flow rate of 10 μL/sec. Increased G2/M-phase arrest can be observed with exposure to pH-engineered ADCs specific for DLL3 as compared to control ABPC ADC specific for DLL3.

For DNA-damaging toxins (e.g., pyrrolobenzodiazepine or “PBD”), DNA damage is assessed by measuring the phosphorylated histone H2AX (γH2AX). H2AX is normally phosphorylated in response to double-strand breaks in DNA; however, increased levels γH2AX may also be observed as a result of treatment with DNA-cross-linking toxins such as PBD or cisplatin (Huang, X. et al. 2004, Cytometry Part A 58A, 99-110). DLL3+ cells are treated with pH-engineered and control ABPC ADCs specific for DLL3 as described herein. After treatment, cells are rinsed with PBS, and then fixed in suspension in 1% methanol-free formaldehyde (Polysciences, Warrington, Pa.) in PBS at 0° C. for 15 min. Cells are resuspended in 70% ethanol for at least 2 h at −20° C. Cells are then washed twice in PBS and suspended in 0.2% Triton X-100 (Sigma) in a 1% (w/v) solution of BSA (Sigma) in PBS for 30 min to suppress nonspecific Ab binding. Cells are centrifuged again (200 g, 5 min) and the cell pellet is suspended in 100 μL of 1% BSA containing 1:800 diluted anti-histone γH2AX polyclonal Ab (Trevigen, Gaithersburg, Md.). The cells are then incubated overnight at 4° C., washed twice with PBS, and resuspended in 100 μL of 1:30 diluted FITC-conjugated F(ab′)2 fragment of swine anti-rabbit immunoglobulin (DAKO, Carpinteria, Calif.) for 30 min at room temperature in the dark. The cells are then counterstained with 5 μg/mL of PI (Molecular Probes, Eugene, Oreg.) dissolved in PBS containing 100 μg/mL of DNase-free RNase A (Sigma), for 20 min at room temperature. Cellular fluorescence of the FITC γH2AX signal and the PI counterstain are measured using flow cytometry using methods known in the art. When comparing cells within the same stage of the cell cycle (based on total DNA content), treated DLL3+ cells can be observed to have an increased FITC γH2AX signal relative to untreated DLL3+ cells (which serve as a baseline). Furthermore, DLL3+ cells treated with pH-engineered ADCs specific for DLL3 can be observed to have a greater increase in levels of γH2AX over baseline than cells treated with a control ABPC ADC specific for DLL3. In addition to the γH2AX assay, DNA cross-linking can be more directly assessed with a Comet assay (Chandna, S. (2004) Cytometry 61A, 127-133).

In addition, as disclosed herein, pH-engineered and control ABPCs can be assayed using the methods in this example without direct conjugation by performing a secondary ADC assay instead of using primary conjugated ADCs.

### Example 6. Demonstration of Decreased Half-Life of pH-Engineered ABPCs Specific for DLL3 as Compared to a Control ABPC Specific for DLL3

One of the surprising aspects of the pH-engineered ABPCs specific for DLL3 described by the invention can be their ability to facilitate increased dissociation of ABPCs from the DLL3 within the endosome or lysosome resulting in a decreased serum half-life relative to control ABPCs specific for DLL3 or ABPCs that are not specific for DLL3. This decreased serum half-life is due to the enhanced frequency with which pH-engineered ABPCs specific for DLL3 are cleared from circulation due to their enhanced cellular internalization once bound to DLL3, which over time can be observed through a decrease in serum concentration of unbound pH-engineered ABPC specific for DLL3. To demonstrate these properties, a series of animal studies in mice and/or monkeys is performed using pH-engineered ABPC specific for DLL3 and control ABPC specific for DLL3 using methods known to the art (e.g., Gupta, P., et al. (2016), mAbs, 8:5, 991-997). Briefly, to conduct mouse studies, a single intravenous bolus (e.g., 5 mg/kg) of either pH-engineered ABPC specific for DLL3 or control ABPC specific for DLL3 is administered via tail vein to two groups of NOD SCID mice (e.g. Jackson Labs NOD.CB17-Prkdcscid/J Stock No: 001303) xenografted with a DLL3+ cell line (e.g., as described herein). Xenografted mice are prepared by growing 1-5 million DLL3+ cells in vitro and inoculating subcutaneously into the right flank of the mouse. Tumors are size matched at 300 mm3. Measurements of the length (L) and width (W) of the tumors are taken via electronic caliper and the volume is calculated according to the following equation: V=L×W\hat{ }2/2. Blood samples are collected via retro-orbital bleeds from each group at each of the following time points: 15 m, 30 m, 1 h, 8 h, 24 h, and 3 d, 7 d, 10 d, 14 d, 17 d, 21 d, and 28 d. Samples are processed to collect serum, and antibody concentrations are quantified using ELISA or other methods known to the art (e.g., PAC assay or MAC assay; Fischer, S. K. et al. (2012), mAbs, 4:5, 623-631, utilizing, e.g., anti-human Fc antibody Jackson ImmunoResearch Labs, Cat #109-006-006). Antibody concentrations of pH-engineered ABPC specific for DLL3 and control ABPC specific for DLL3 are plotted as a function of time. Upon analysis of the data, it can be observed that the pH-engineered ABPC specific for DLL3 has a significantly shorter serum half-life relative to control ABPC specific for DLL3, thereby demonstrating the ability of the pH-engineered ABPC specific for DLL3's pH dependence to facilitate an enhanced dissociation within the endosome or lysosome relative to other, similar binders (e.g., control ABPC specific for DLL3) that bind the same antigen but that differ in their pH dependence. If the pH-engineered and control ABPCs specific for DLL3 are cross-reactive with the mouse homolog of DLL3, a similar experiment can be repeated with non-xenografted mice.

Optionally, if the pH-engineered and control ABPCs specific for DLL3 are cross-reactive with the cynomolgus monkey homolog of DLL3, a similar experiment can be performed on monkeys (e.g., cynomolgus monkeys). An equal number of male and female monkeys (e.g., n=1-2 each) are administered a bolus of either pH-engineered ABPC specific for DLL3 or control ABPC specific for DLL3 at a dose of, e.g., 1 mg/kg via saphenous vein injection. Alternatively, several different doses of DLL3-binding protein are administered across a group of several monkeys. Blood samples are collected via the peripheral vein or femoral vein at intervals similar to those described above, and analyzed for the presence of either pH-engineered ABPC specific for DLL3 or control ABPC specific for DLL3 using methods known to the art (e.g., ELISA). Upon analysis of the data, it can be observed that pH-engineered ABPC specific for DLL3 has a significantly shorter serum half-life relative to control ABPC specific for DLL3, thereby demonstrating the ability of pH-engineered ABPC specific for DLL3 to facilitate an enhanced dissociation within the endosome or lysosome relative to other, similar binders (e.g., control ABPC specific for DLL3) that bind the same antigen but that differ in their pH dependence. In some cases, this effect is observed only in certain doses, whereas in others it is observed across doses.

In addition, the half-life of pH-engineered and control ABPC ADCs specific for DLL3 can be assessed using the above methods by substituting pH-engineered and control ABPC ADCs specific for DLL3 for the pH-engineered and control ABPCs specific for DLL3 (i.e., studying the ABPCs after conjugation to a drug or toxin, as described herein).

### Example 7. Increased Potency of pH-Engineered ADCs Specific for DLL3 vs. A Control ABPC ADC Specific for DLL3 in Mouse Xenograft Models

The enhanced anti-tumor activity of the pH-engineered ADCs specific for DLL3 against DLL3+ tumors can be demonstrated in a subcutaneous xenograft model of DLL3+ cells. For the experiments, 1-5 million DLL3+ cells are grown in vitro and inoculated subcutaneously per mouse into the right flank of female immunodeficient (e.g., SCID-Beige or NOD scid) mice. Tumors are size matched at 100-200 mm3, and dosed intraperitoneally (IP) (1 dose given every ˜4-7 days for a total of ˜2-6 doses). Measurements of the length (L) and width (W) of the tumors are taken via electronic caliper and the volume is calculated according to the following equation: V=L×W\hat{ }2/2. A bolus (e.g., 5 mg/kg) of either pH-engineered ADC specific for DLL3 or control ABPC ADC specific for DLL3 is administered via tail vein. Tumor growth inhibition (TGI) and tumor growth delay (TGD) and survival are significantly improved with administration of pH-engineered ADC specific for DLL3 compared to administration of control ABPC ADC specific for DLL3 at the same regimen.

Optionally, spread of tumor cells into the various tissues is determined in sacrificed animals. Metastasis is measured according to Schneider, T., et al., Clin. Exp. Metas. 19 (2002) 571-582. Briefly, tissues are harvested and human Alu sequences are quantified by real-time PCR. Higher human DNA levels, quantified by real-time PCR, correspond to higher levels of metastasis. Levels of human Alu sequences (correlating to invasion of tumor cells into secondary tissue) are significantly lower in animals treated with pH-engineered ADC specific for DLL3, corresponding to reduced metastasis, compared to mice treated with control ABPC ADC specific for DLL3 at the same regimen. Alternatively, the enhanced anti-tumor activity of the pH-engineered ADC specific for DLL3 can be shown in DLL3+ patient-derived xenograft models (e.g., available from Charles River Laboratories).

### Example 8. Creation of a pH-Engineered Bispecific DLL3 Bispecific ABPC and Demonstration of Exemplary Properties as Compared to a Control Bispecific ABPC

To create a pH-engineered ABPC specific for DLL3 with modified toxicity and internalization properties, a bispecific antibody that binds two different epitopes on DLL3 is constructed. It is known in the art that biparatopic antibodies can show increased antigen-dependent internalization, and are therefore useful for applications such as antibody-drug conjugates (e.g., see Li et al (2016) A Biparatopic HER2-Targeting Antibody-Drug Conjugate Induces Tumor Regression in Primary Models Refractory to or Ineligible for HER2-Targeted Therapy, Cancer Cell 29:117-29). Briefly, a pH-engineered DLL3×DLL3 bispecific, biparatopic ABPC specific for DLL3 is assembled using light chain/heavy chain pairs from two different pH-engineered ABPCs specific for DLL3, each of which binds a distinct epitope on DLL3 that does not overlap with the other epitope. A set of pH-engineered ABPCs specific for DLL3 that bind non-overlapping epitopes are discovered, e.g., using the methods described herein, or others known to one of ordinary skill in the art. Briefly, two binders are selected on the basis that they bind substantially different epitopes on DLL3, as determined by, e.g., a binding competition assay as in Abdiche Y N et al (2009) Exploring blocking assays using Octet, ProteOn, and Biacore biosensors, Anal Biochem 386:172-80. Alternatively, briefly, as described herein, cell culture supernatants of cells transfected with a first ABPC specific for DLL3 are normalized to an antibody expression level of 50 μg/mL, and captured on an anti-human Fc sensor (Forte Bio). A baseline is established using 1× kinetics buffer (Forte Bio), and the sensor is associated with 50 nM of DLL3 in 1×PBS (that has been mixed and pre-incubated for 30 min at 37 degrees C. with a second ABPC specific for DLL3 transfection supernatant or the first ABPC specific for DLL3 transfection supernatant, both normalized to 50 ug/mL) at pH 7.4 for 300 sec to generate an association curve. If the association rate in the presence of the second ABPC specific for DLL3 is significantly faster (as calculated by the instrument software, or as seen by an elevated level of association over time) than the association rate in the presence of the first ABPC specific for DLL3, then the second ABPC specific for DLL3 is deemed to bind a non-overlapping epitope of DLL3. Optionally, each antibody is screened for its internalization properties when bound to its epitope on a cell expressing DLL3, and well-internalizing antibodies are selected. Assays for determining the internalization rate of a molecule present on the surface of a cell are known to the art. See, e.g., Wiley et al. (1991) J. Biol. Chem. 266: 11083-11094; and Sorkin and Duex (2010) Curr. Protoc. Cell Biol. Chapter, Unit-15.14; Vainshtein et al. (2015) Pharm Res. 32: 286-299. Once selected, heavy and light chain constructs with engineered mutations for heavy and light chain pairing (Spiess et al., “Alternative molecular formats and therapeutic applications of bispecific antibodies,” 2015) are synthesized for both arms. Bispecific ABPCs specific for DLL3 are produced by co-expression of corresponding heavy and light chain plasmids in, e.g., Expi293 cells. Cell culture supernatants are harvested and subjected to Protein A purification. Heterodimeric ABPCs specific for DLL3 are separated from homodimeric species via additional purification steps such as ion exchange chromatography, hydrophobic interaction chromatography, and mixed mode chromatography. The purified pH-engineered DLL3×DLL3 bispecific, biparatopic ABPCs specific for DLL3 are characterized via mass spectrometry to confirm the purity and absence of homodimeric species and size exclusion chromatography to confirm the presence of monomeric antigen-binding protein construct species. For the product antibody, binding to the DLL3 is confirmed via Biacore analysis. Other methods of bispecific antibody production are known to the art and could also be used to create a bispecific antibody, e.g., the DLL3×DLL3 bispecific, biparatopic ABPCs specific for DLL3 described herein (e.g., Labrijn et al (2014) “Controlled Fab-arm exchange for the generation of stable bispecific IgG1” Nature Protocols 9:2450-2463, accessed at http://www.nature.com/nprot/journal/v9/n10/abs/nprot.2014.169.html), as would be apparent to one of ordinary skill in the art. Alternatively, instead of a DLL3×DLL3 ABPC specific for DLL3, a pH-engineered DLL3× BINDER ABPC specific for DLL3 can be constructed using similar methods apparent to one skilled in the art, where BINDER is any antibody that has been published in the art or discovered using methods like those herein or those known in the art (e.g., display-based or immunization-based methods).

Next, exemplary properties of pH-engineered DLL3×DLL3 ABPCs specific for DLL3 can be demonstrated using the methods described herein, with the appropriate control being a control ABPC monospecific or bispecific ABPC specific for DLL3. Briefly, it can be shown that, as compared to a control, the pH-engineered DLL3×DLL3 ABPCs specific for DLL3: a) bind in a pH-dependent manner to cells, e.g., bind at a neutral pH but not an acidic pH and b) release from cells in a pH-dependent manner, e.g. bind at a neutral pH and release at an acidic pH and c) show enhanced endolysosomal accumulation in DLL3+ cells and d) show increased DLL3 antigen density after exposure to DLL3+ cells and e) when conjugated to a toxin, show increased cytotoxicity to DLL3+ cells and f) when conjugated to a toxin, show increased toxin liberation when incubated with DLL3+ cells and g) show decreased half-life when exposed to DLL3 antigen in a relevant animal model and h) when conjugated to a toxin, show increased efficacy in a mouse xenograft model of DLL3+ cells. Similarly, the exemplary properties of pH-engineered DLL3× BINDER ABPCs specific for DLL3 can be demonstrated using the methods described herein, with the appropriate control being a control ABPC DLL3× BINDER bispecific ABPC specific for DLL3.

### Example 9. Construction and Screening of pH-Engineered DLL3 ABPCs

Multiple DLL3-binding monoclonal antibodies have been described in the literature and can be used as a template for engineering pH-dependent binding [Saunders L R (2016) “A DLL3-targeted antibody-drug conjugate eradicates high-grade pulmonary neuroendocrine tumor-initiating cells in vivo” Science Translational Medicine 7(302):302ra136]. We selected rovalpituzumab (Heavy chain SEQ ID NO: 1, Light chain SEQ ID NO: 2) as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT0639-MYT0679). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE analysis, and the pH dependence of the variant was evaluated using biolayer interferometry (BLI) on an Octet RED 384 instrument. Briefly, cell culture supernatants were normalized to an antibody expression level of 25 μg/mL, and captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of DLL3 (R&D Systems, Cat #9749-DL) in 1×PBST pH 7.4 for 120 sec to generate an association curve. In the dissociation phase, the antibody-antigen complex on the sensor was exposed to 1×PBST pH 7.4 for 300-600 sec. Baseline, association, and dissociation were repeated using 1×MES (100 mM MES Buffer+150 mM NaCl+0.05% Tween 20) pH 5.0 throughout in a separate condition. Association and dissociation phase curves were examined for the starting ABPC antibody and each corresponding antibody variant at pH 5.0 and pH 7.4 to inform on two criteria: a) enhanced dissociation (i.e., higher koff values) at pH 5.0 due to histidine or alanine substitution compared to the starting ABPC, and b) reduced dissociation at pH 7.4 (i.e., lower koff values) compared to pH 5.0 in the antibody variant itself and with the starting ABPC. Heavy chain variants that showed either enhanced dissociation at pH 5.0 or reduced dissociation at pH 7.4 or both were selected for further analysis (e.g., MYT0643, MYT0645, MYT0647-MYT0648, MYT0650-MYT0652, MYT0655-MYT0657, MYT0660, MYT0669-MYT0670, MYT0673, MYT0675, MYT0677, MYT0678). It was also noted that while some histidine and alanine mutations obliterated DLL3 binding (e.g., MYT0649, MYT0654, MYT0671-MYT0672, MYT0676), others were tolerated with little (e.g., less than 1-fold change in dissociation constant KD or dissociation rate) or no change in DLL3 binding kinetics (e.g., MYT0639-MYT0642, MYT0644, MYT0646, MYT0653, MYT0658-MYT0659, MYT0661-MYT0668, MYT0674, MYT0679). Especially because histidine is a large, positively charged amino acid, these variants with no change were noted as positions in the heavy chain that may tolerate a wide range of mutations and lead to antibodies with different sequence but similar binding properties, a designation that is not otherwise apparent.

Additionally, the above protocol was repeated except as follows. A baseline was established using 1×PBST (50 mM Potassium Phosphate Buffer+150 mM NaCl+0.05% Tween 20) pH 7.4, and the sensor was associated with 50 nM of DLL3 in 1×PBST pH 7.4 for 120 sec to generate an association curve. In the dissociation phase, the antibody-antigen complex on the sensor was exposed to 1×PBST pH 7.4 for 300-600 sec. Baseline, association, and dissociation were repeated using 1×PBST pH 5.4 throughout in a separate condition.

### Example 10. Construction and Screening of pH-Engineered DLL3 ABPCs

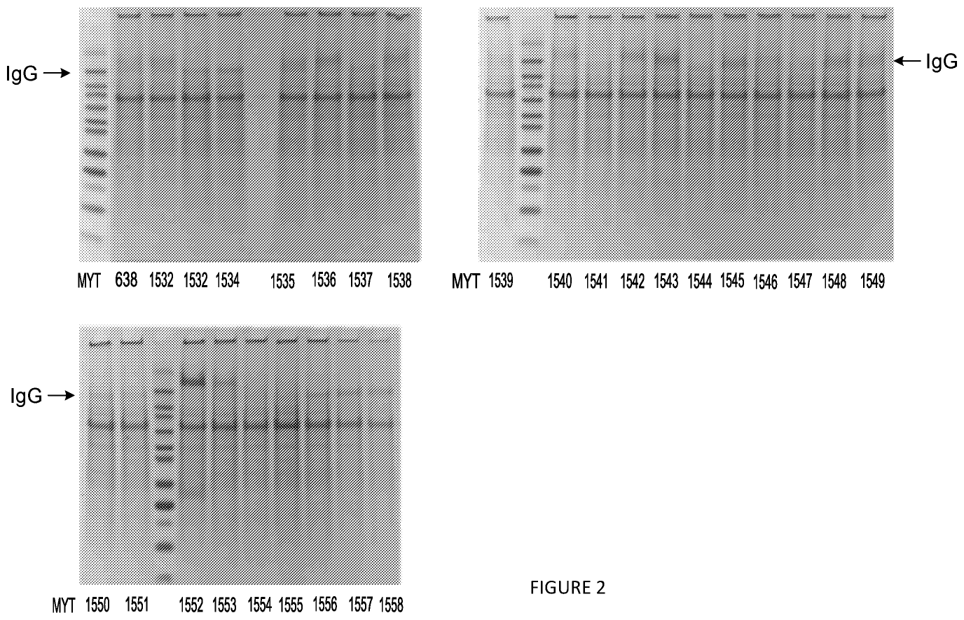

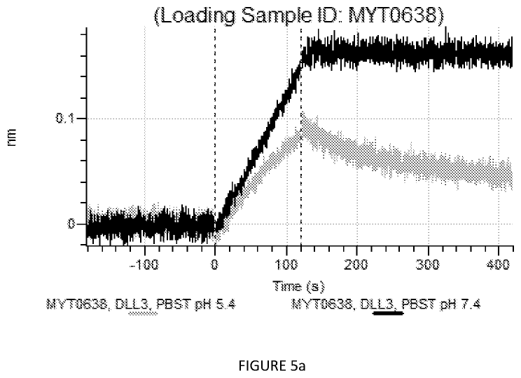

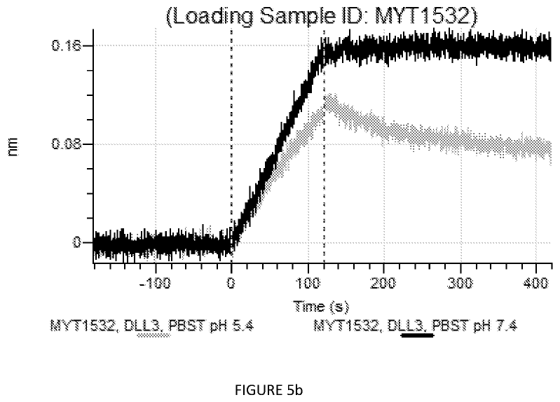

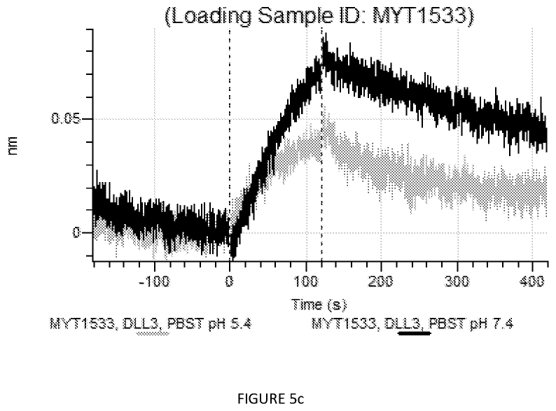

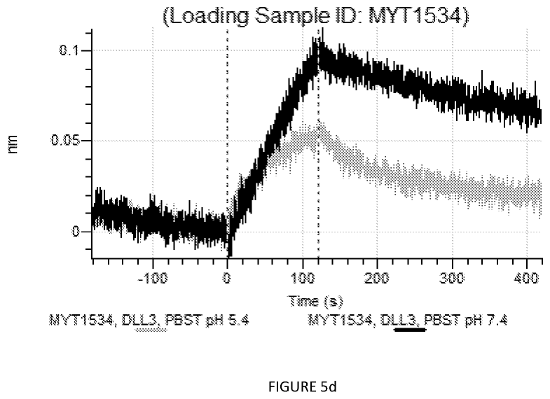

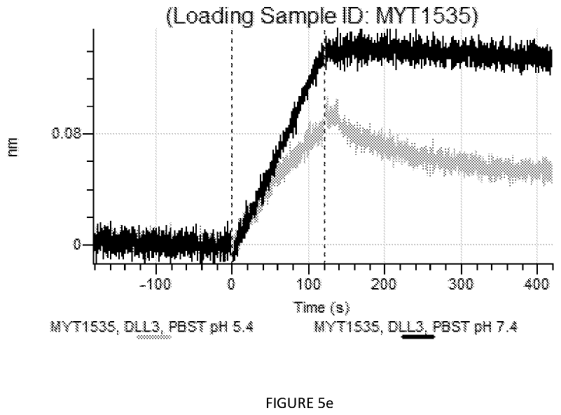

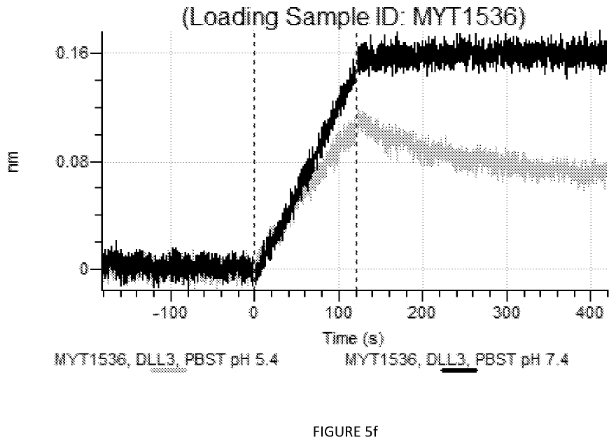

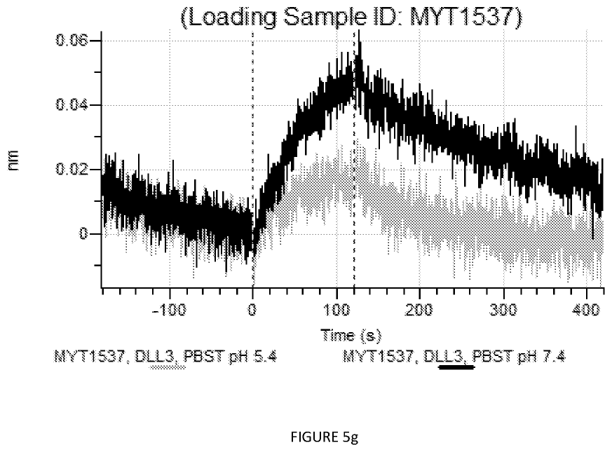

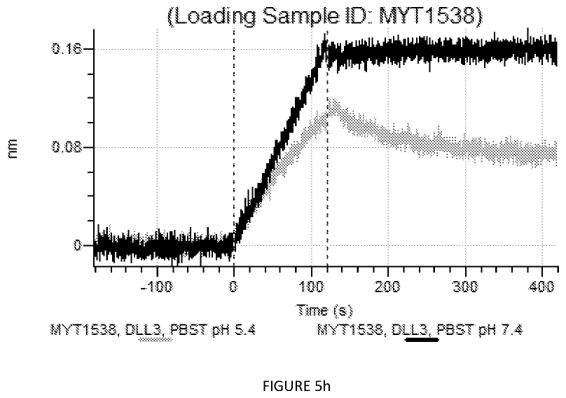

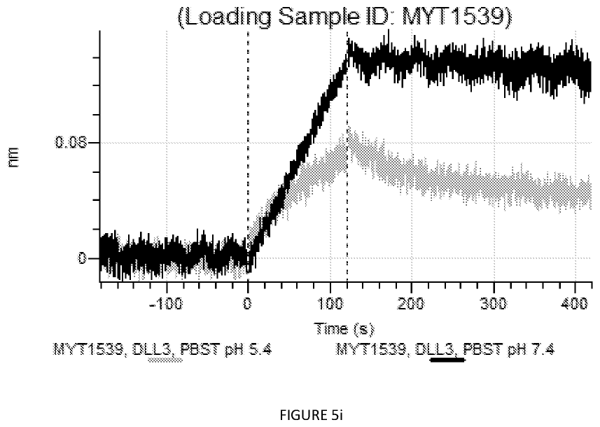

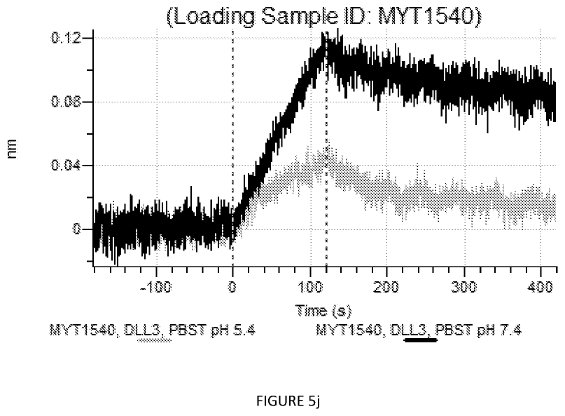

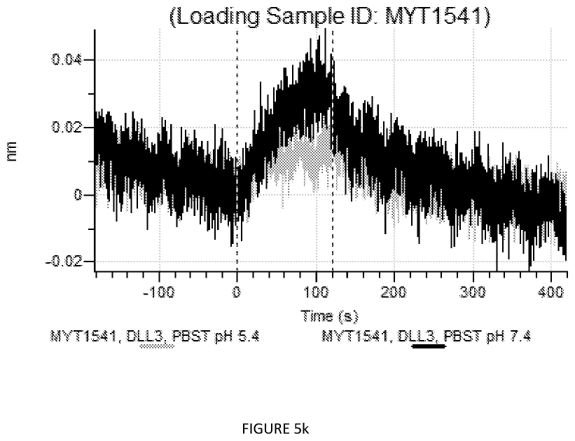

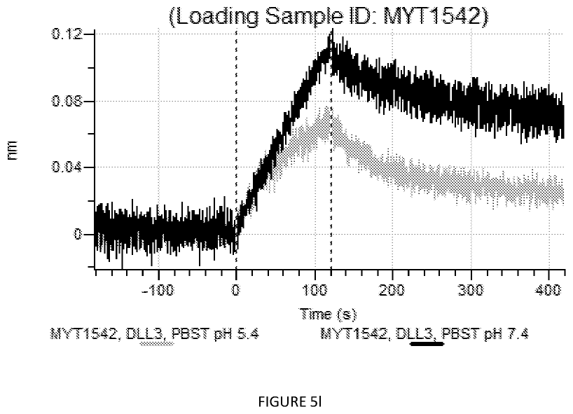

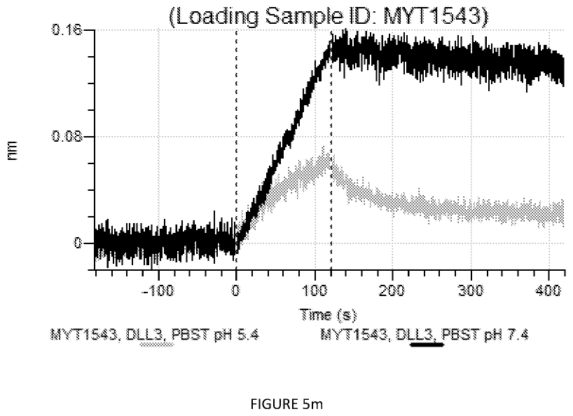

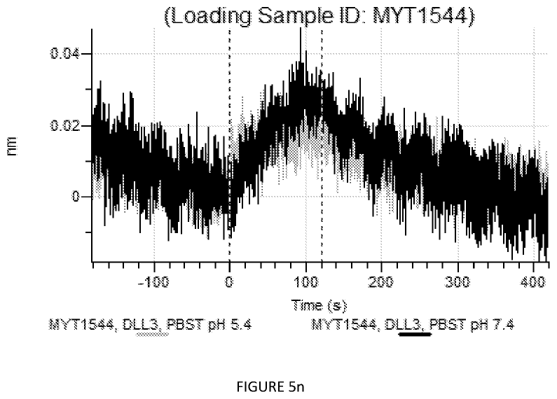

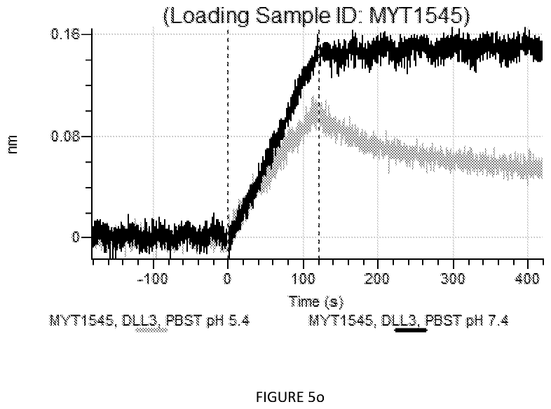

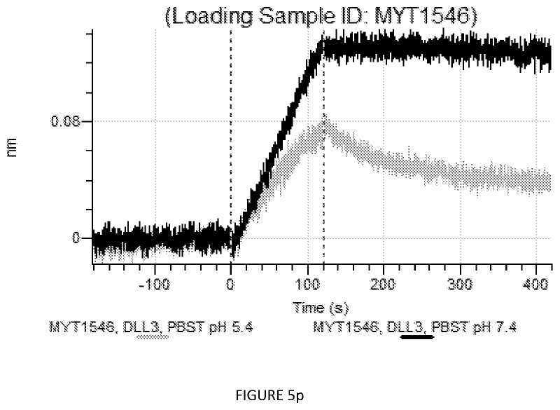

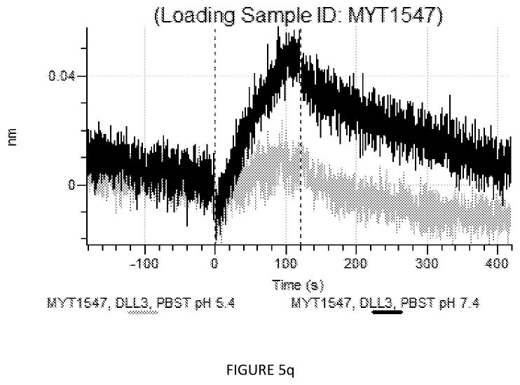

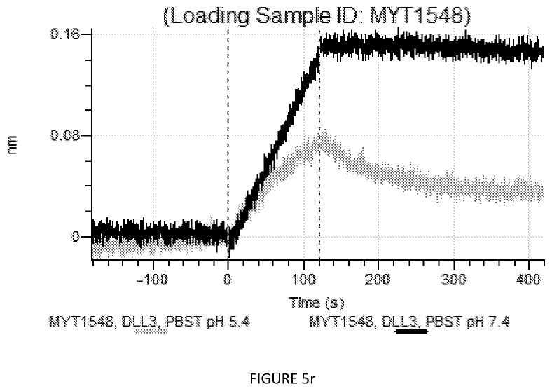

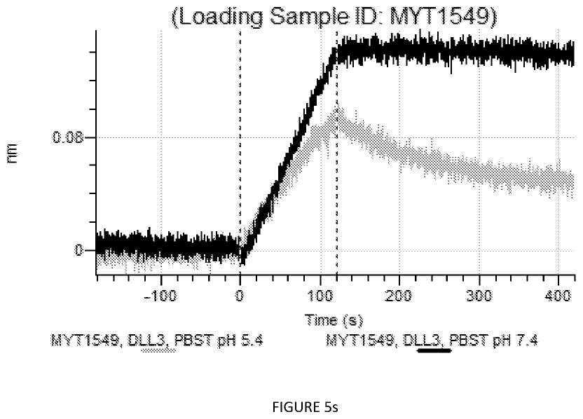

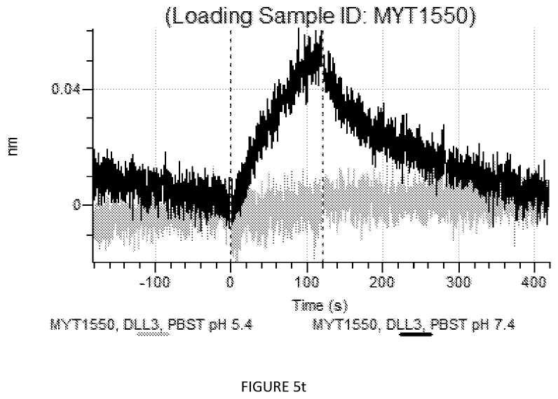

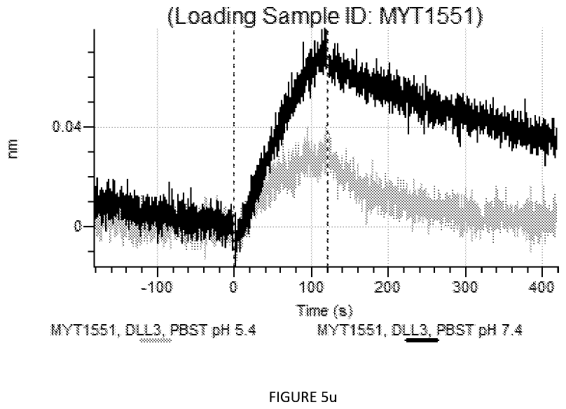

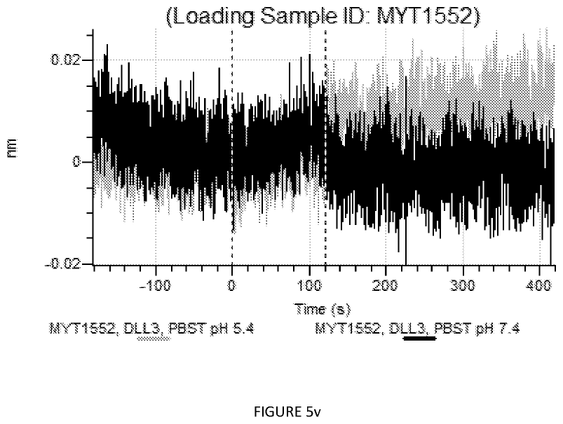

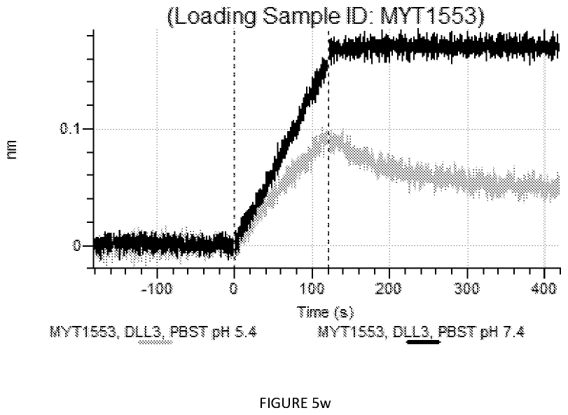

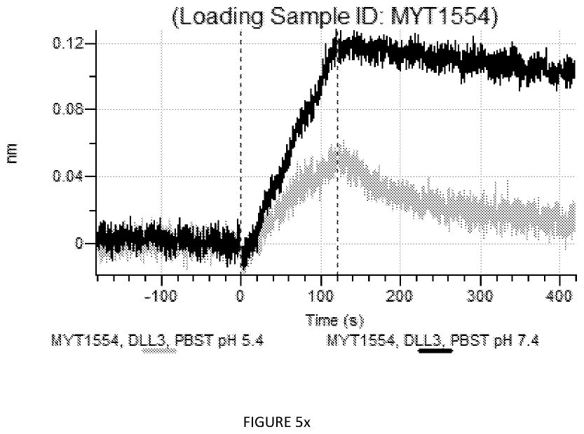

Multiple DLL3-binding monoclonal antibodies have been described in the literature and can be used as a template for engineering pH-dependent binding [Saunders L R (2016) “A DLL3-targeted antibody-drug conjugate eradicates high-grade pulmonary neuroendocrine tumor-initiating cells in vivo” Science Translational Medicine 7(302):302ra136]. ROVALPITUZUMAB (Heavy chain SEQ ID NO: 1, Light chain SEQ ID NO: 2) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT1532-MYT1558). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 2), and the pH dependence of the variant was evaluated using biolayer interferometry (BLI) on an Octet RED 384 instrument. Briefly, 5 μL of cell culture supernatant was diluted into 195 μL of 1×PBST pH 7.4 for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of DLL3 (R&D Systems, Cat #9749-DL, Lot No. DFSG0719011) in 1×PBST pH 7.4 for 120 sec to generate an association curve. In the dissociation phase, the antibody-antigen complex on the sensor was exposed to 1×PBST pH 7.4 for 300-600 sec. Baseline, association, and dissociation were repeated using 1×PBST pH 5.4 throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 5 were selected for further analysis (e.g., MYT1533, MYT1534, MYT1537, MYT1540, MYT1541, MYT1542, MYT1544, MYT1547, MYT1550, MYT1551, MYT1554, MYT1555, MYT1556, MYT1557). It was also noted that while some histidine and alanine mutations obliterated DLL3 binding (e.g., MYT1552), others were tolerated with little (e.g., less than 1-fold change in dissociation constant KD or dissociation rate) or no change in DLL3 binding kinetics (e.g., MYT1532, MYT1535, MYT1536, MYT1538, MYT1539, MYT1543, MYT1545, MYT1546, MYT1548, MYT1549, MYT1553, MYT1558). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 11. Construction and Screening of pH-Engineered DLL3 ABPCs

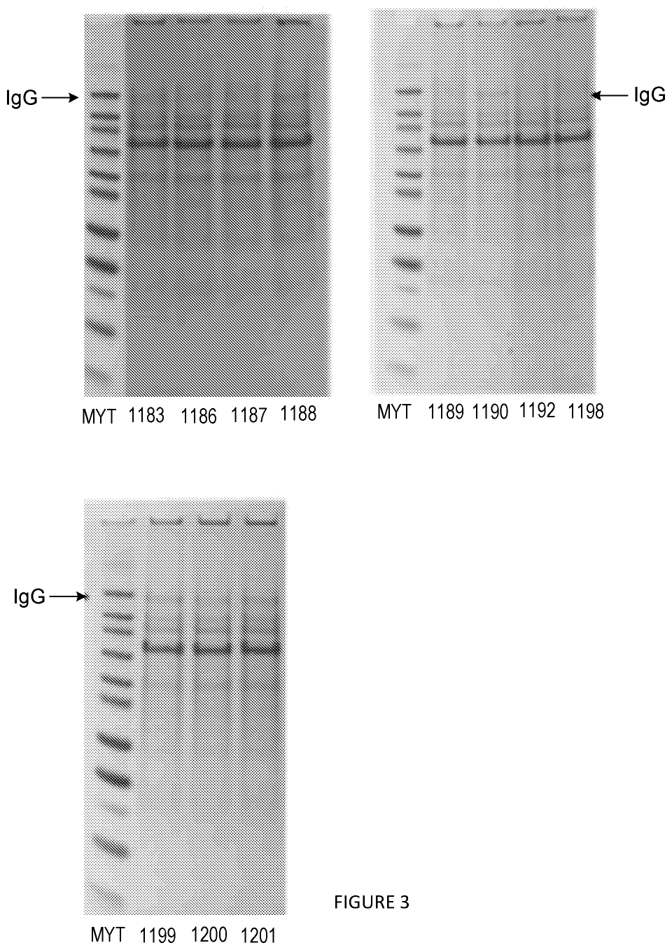

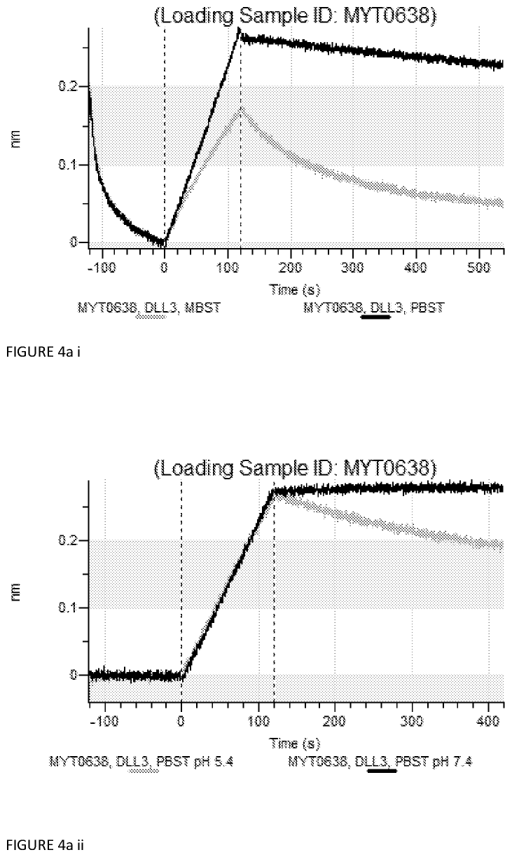

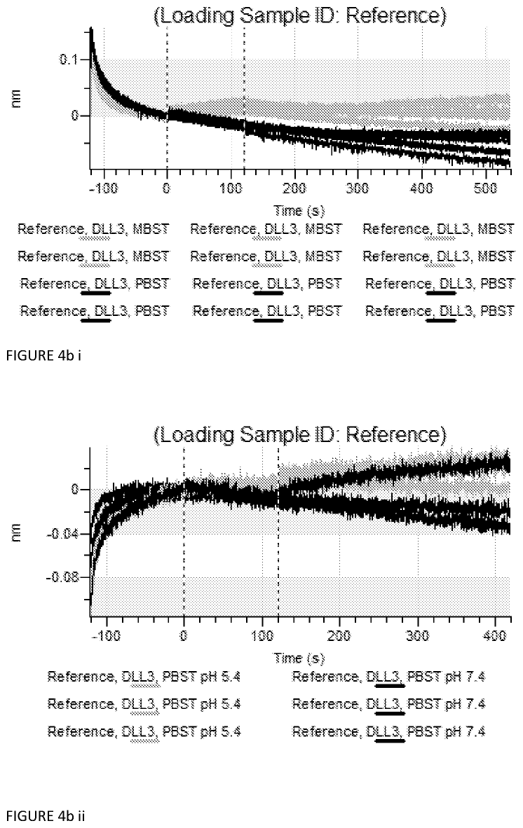

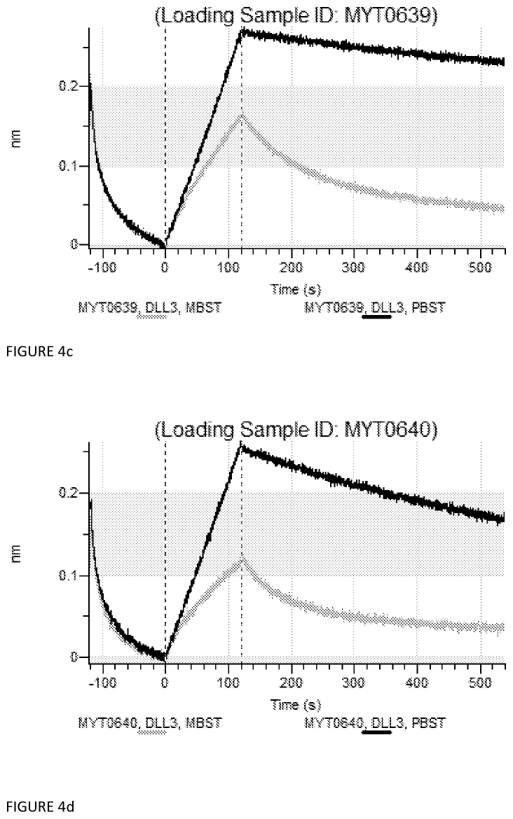

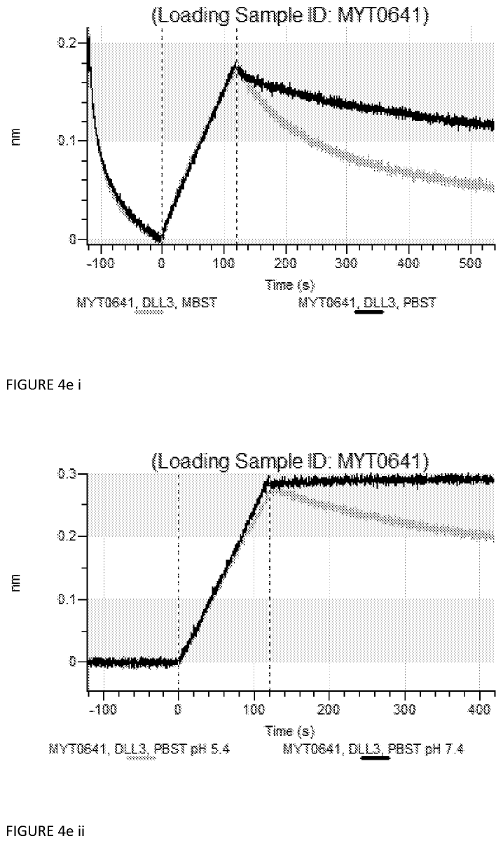

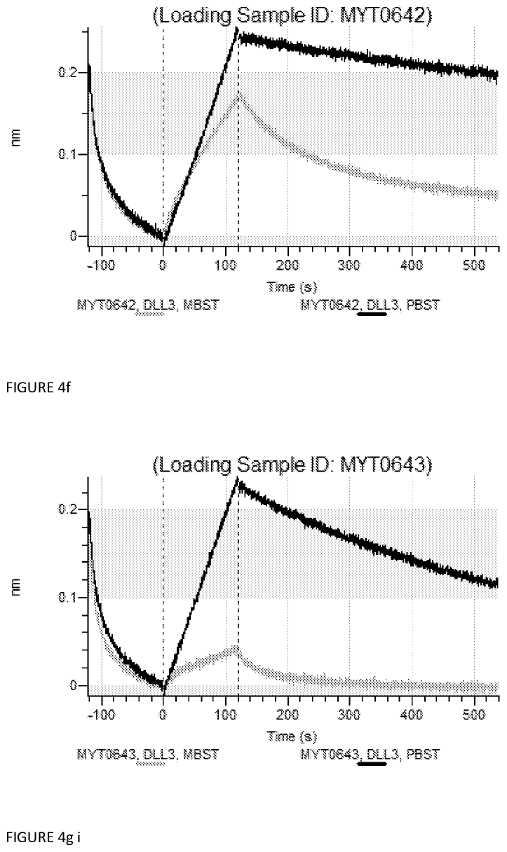

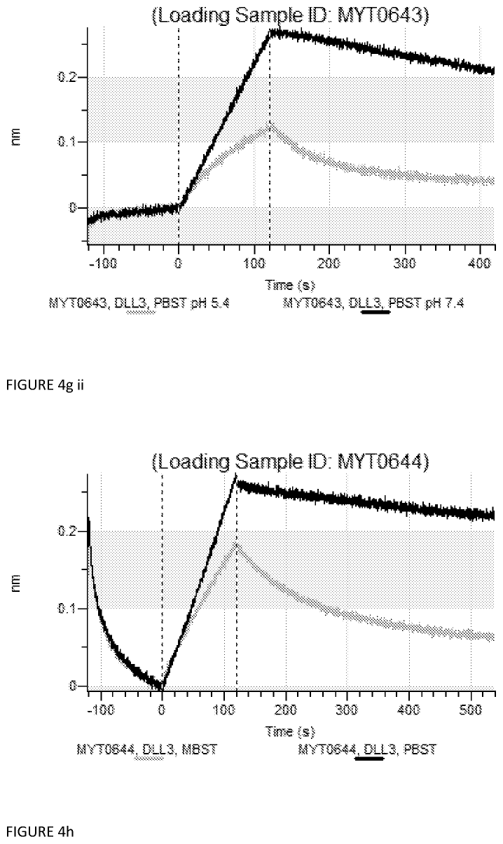

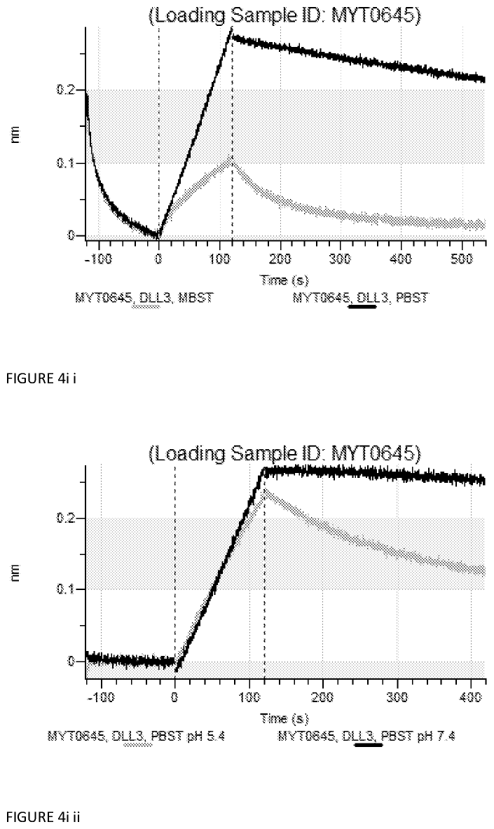

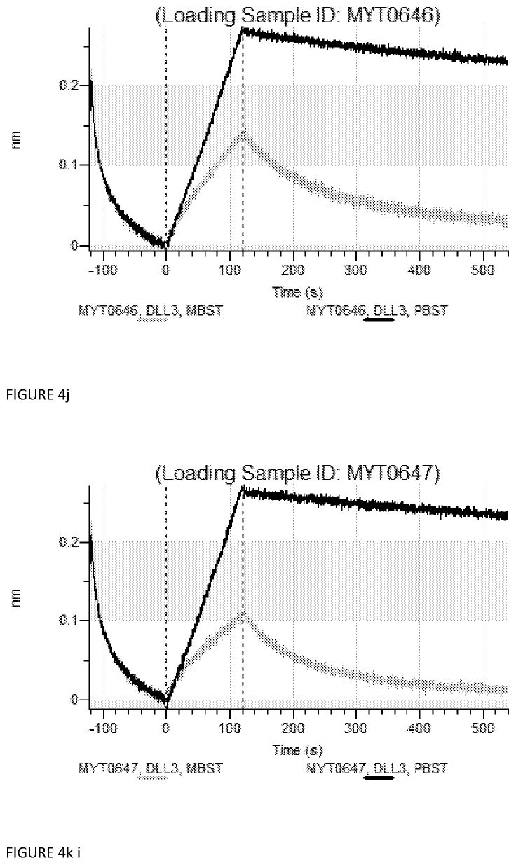

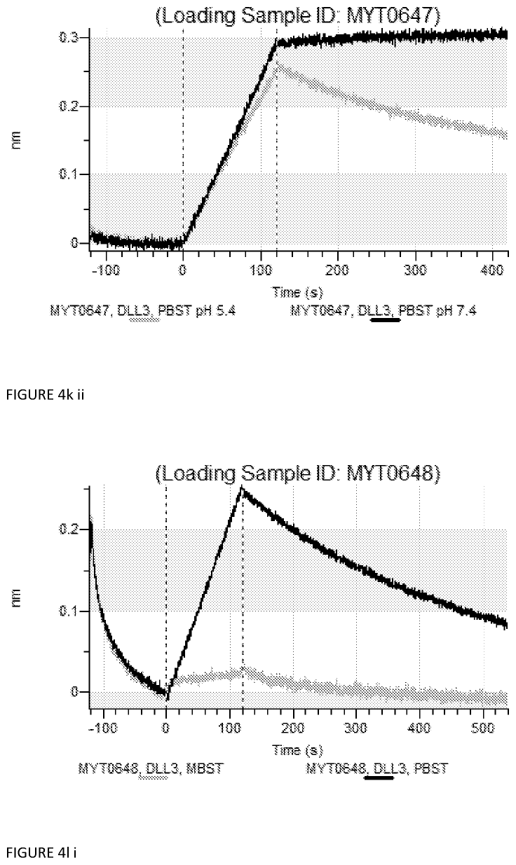

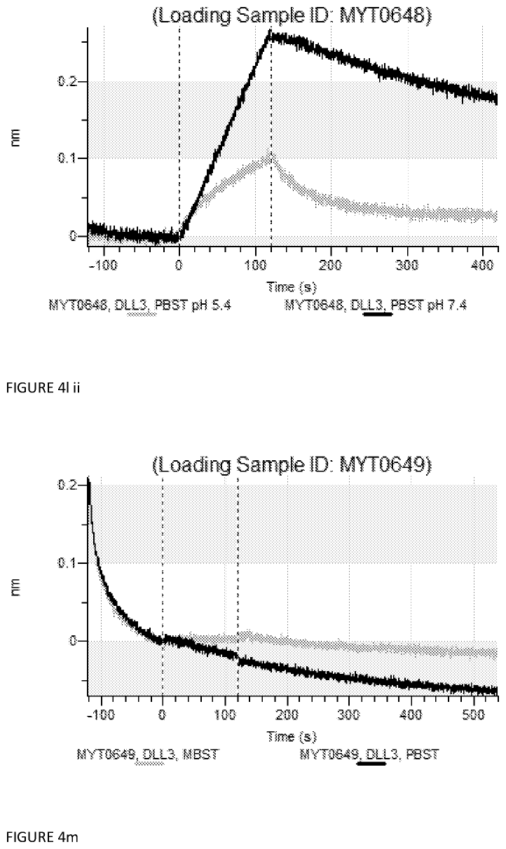

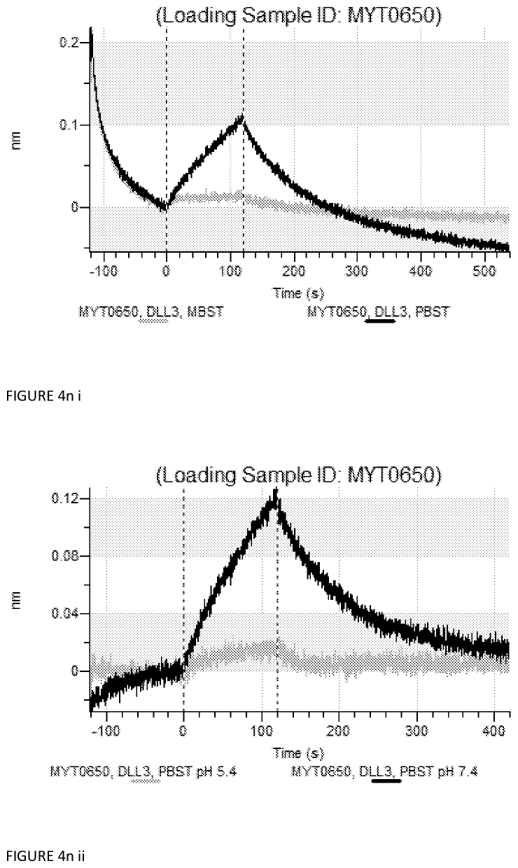

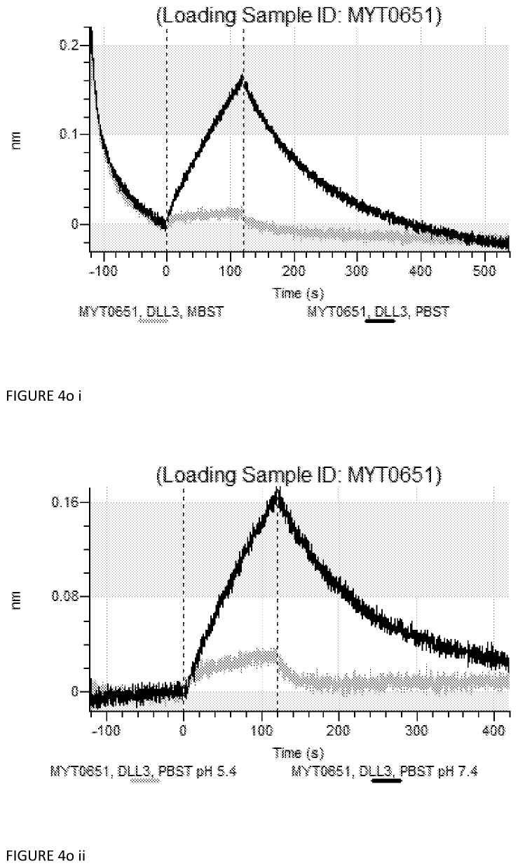

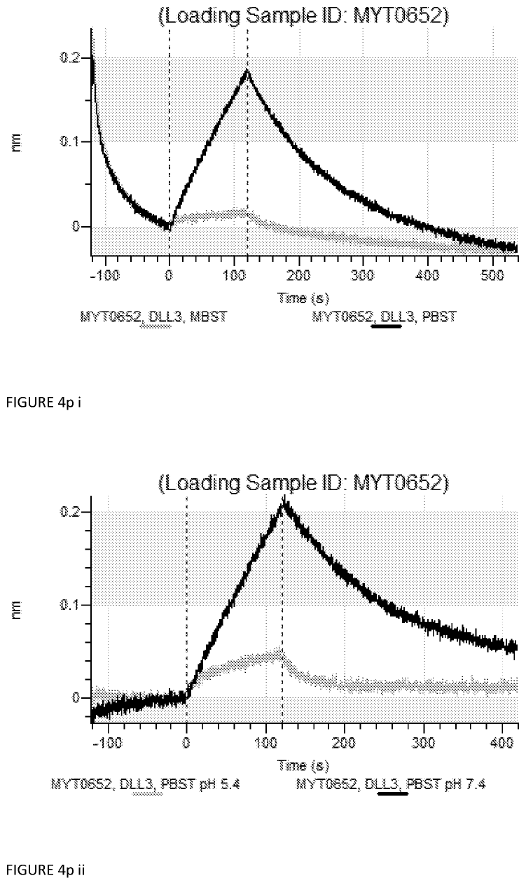

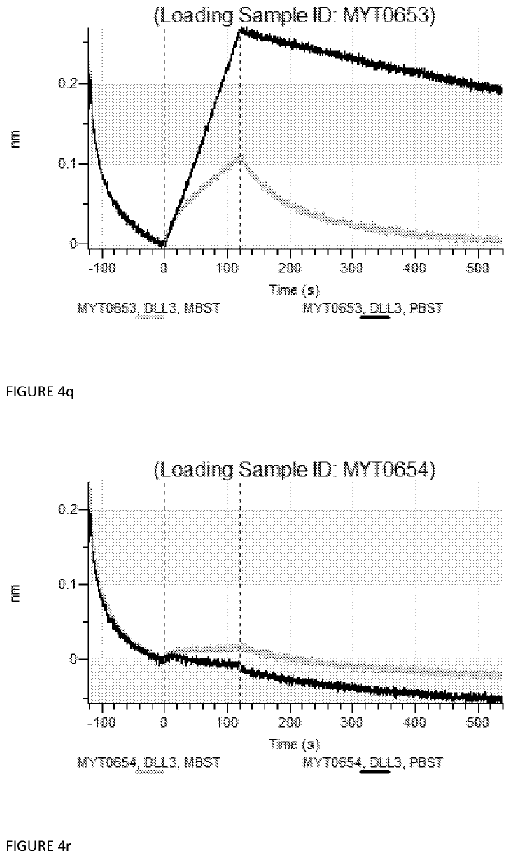

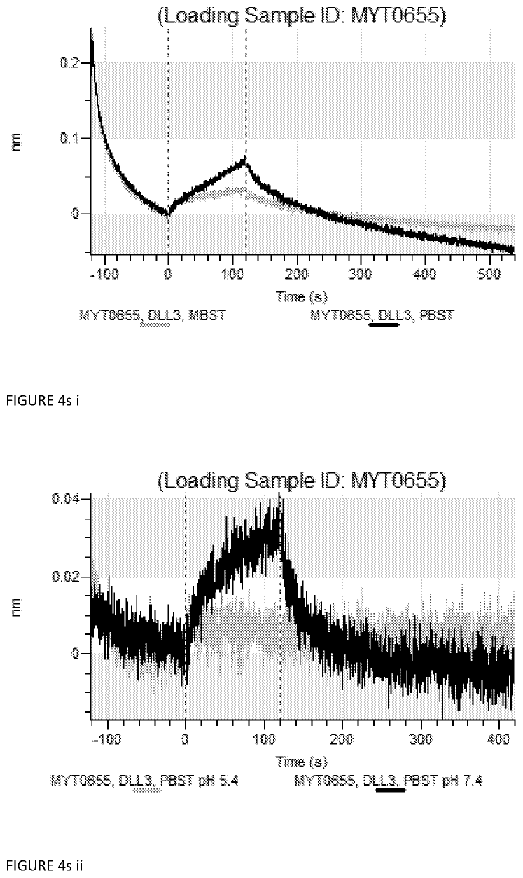

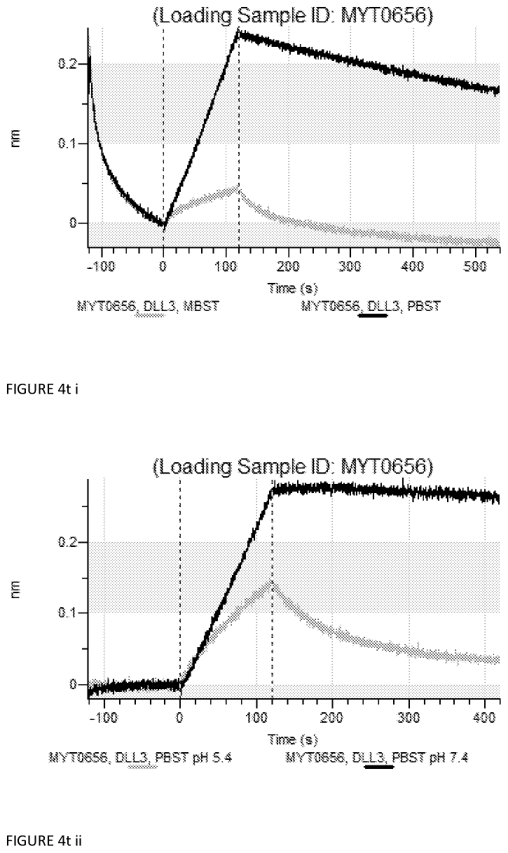

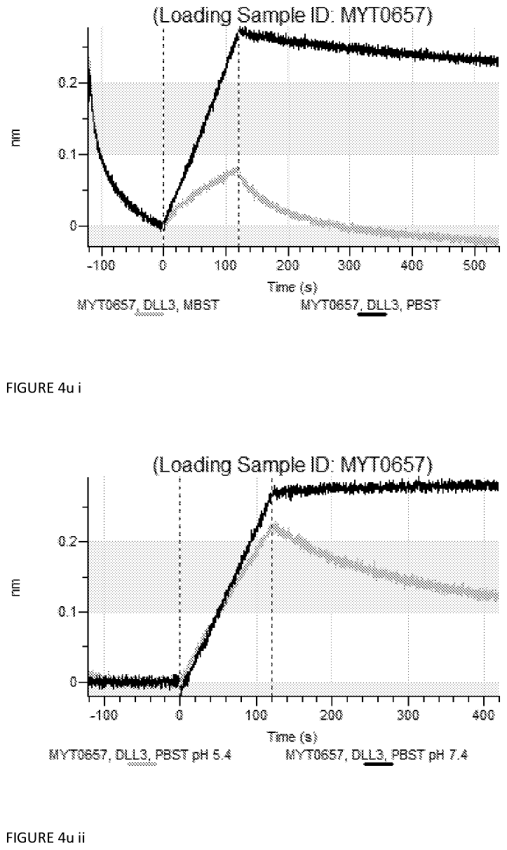

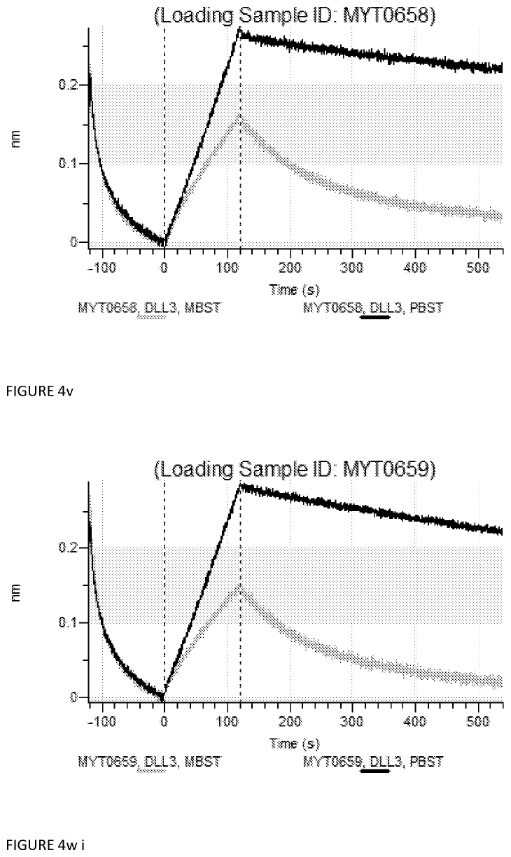

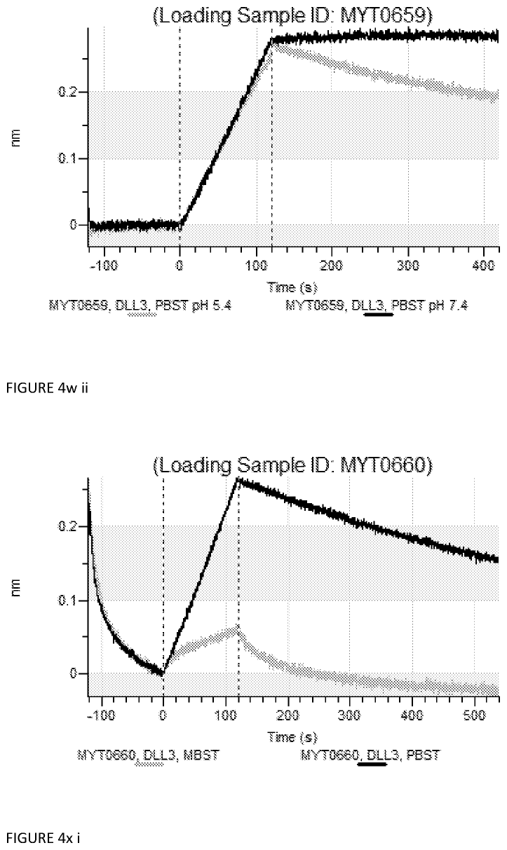

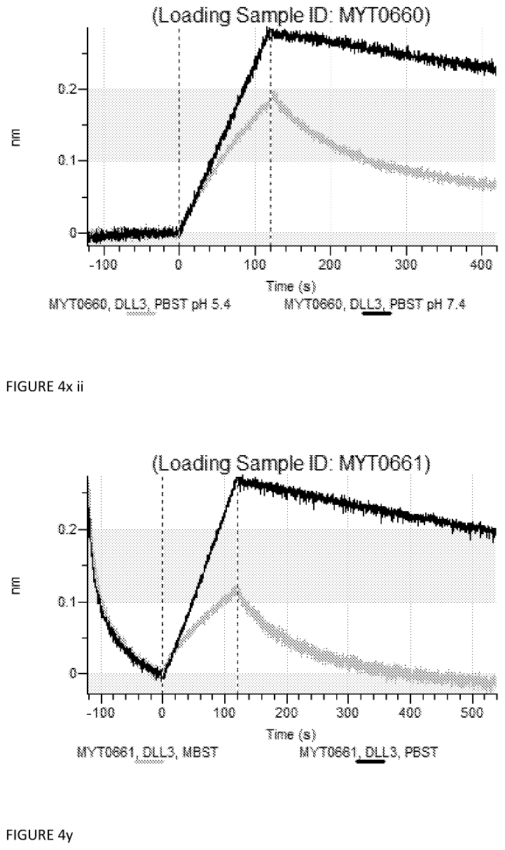

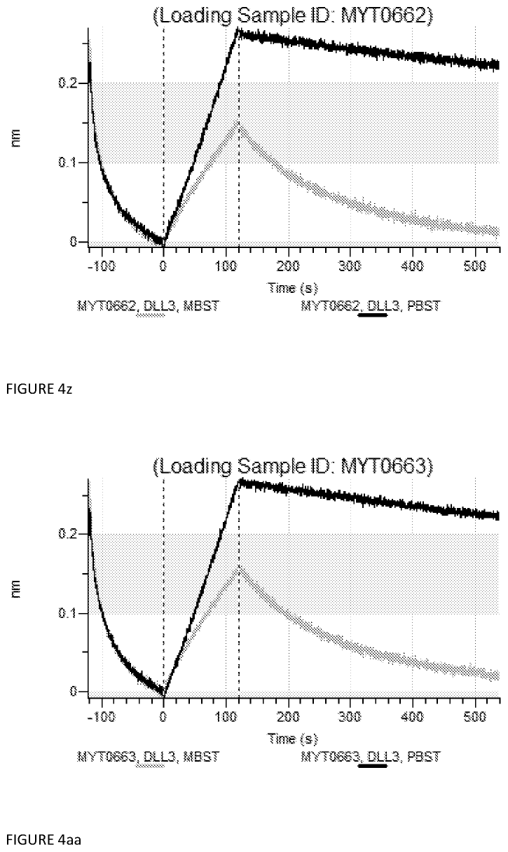

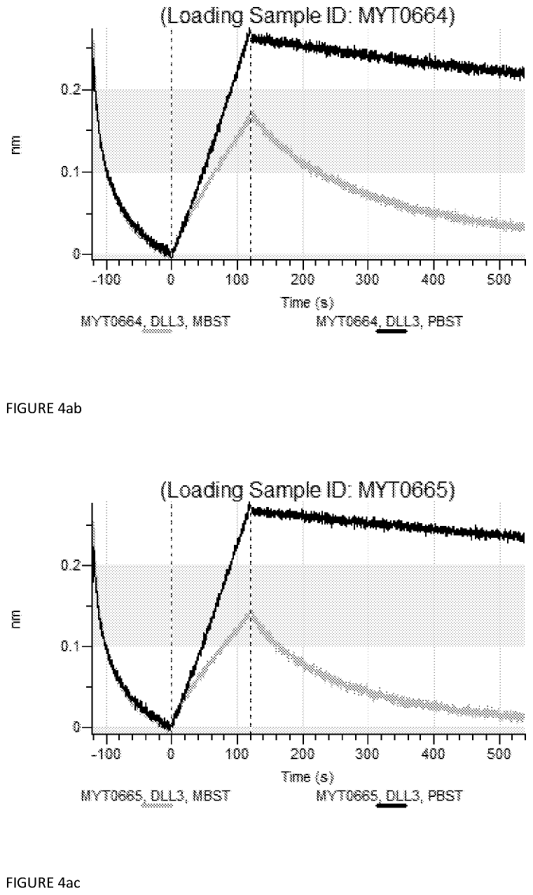

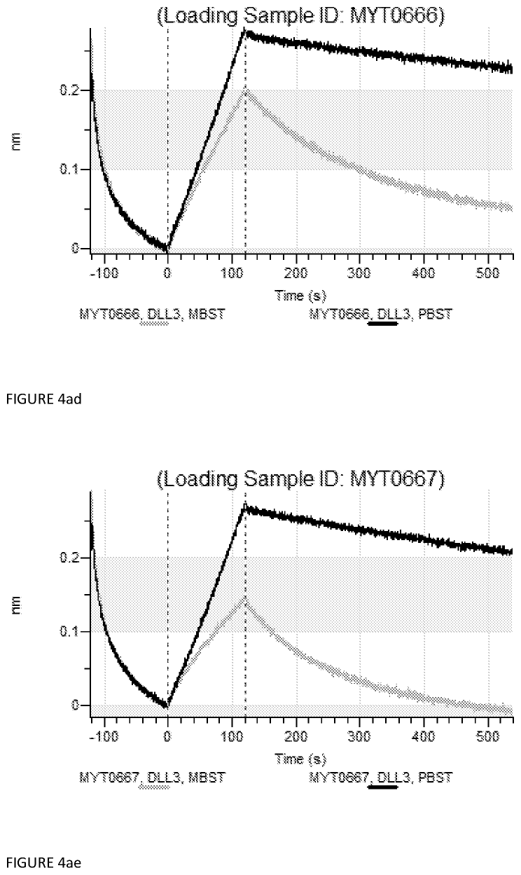

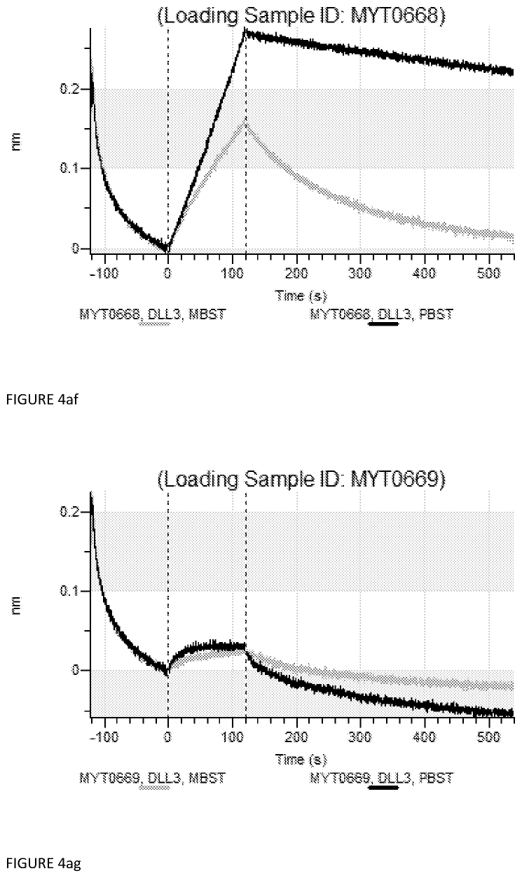

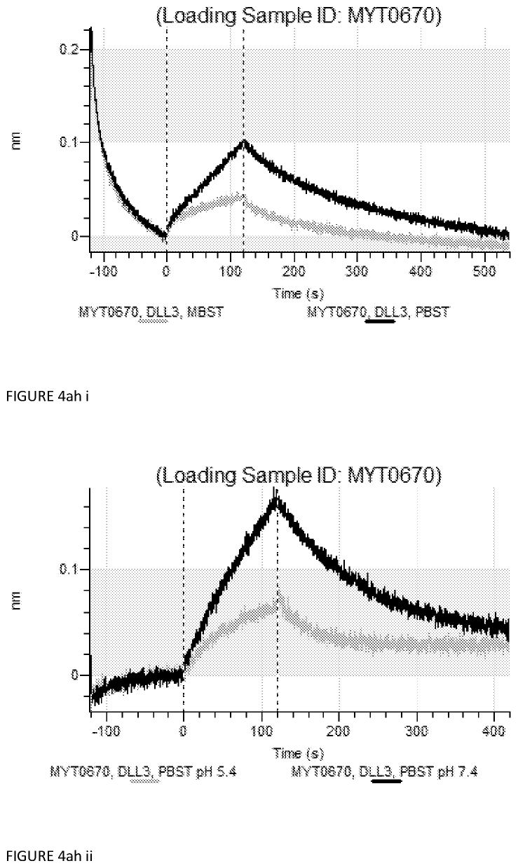

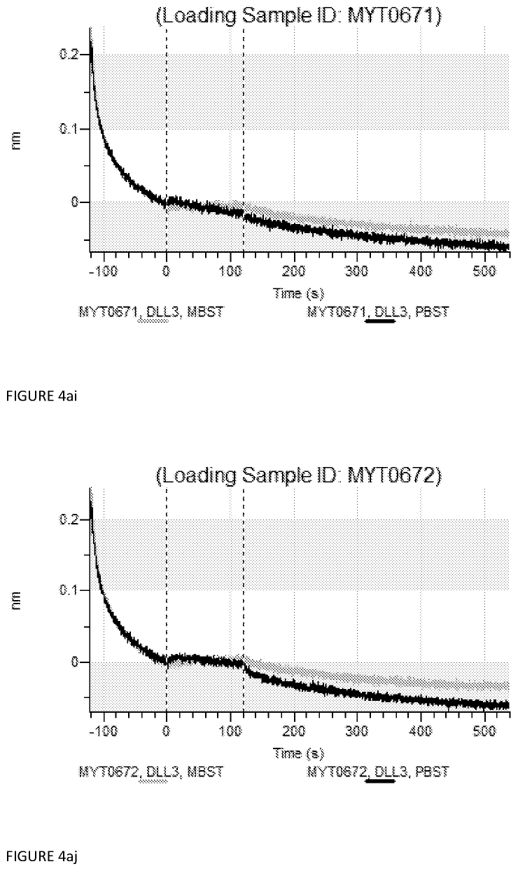

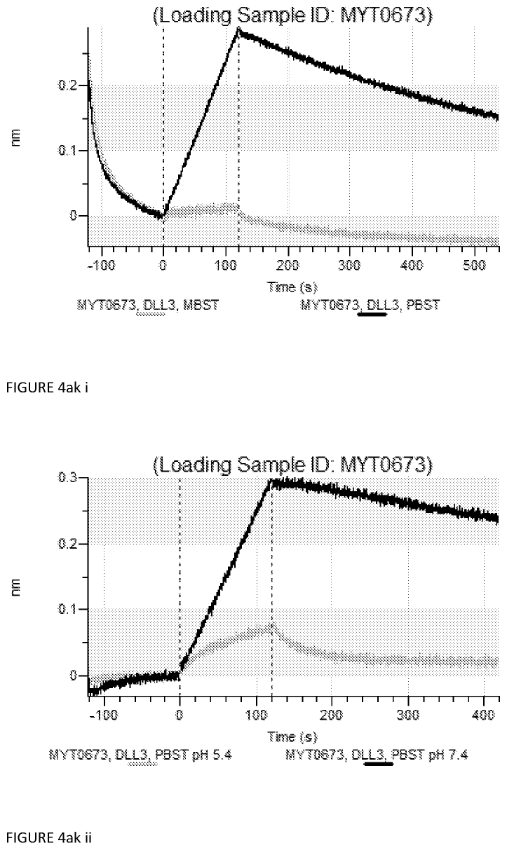

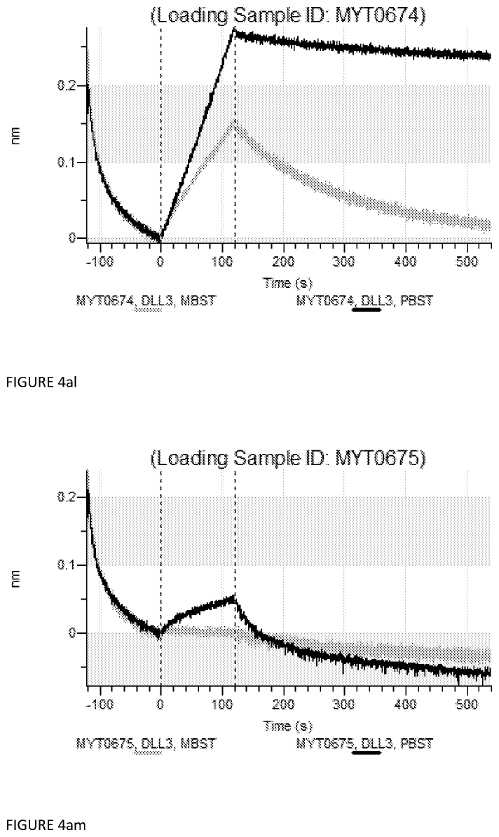

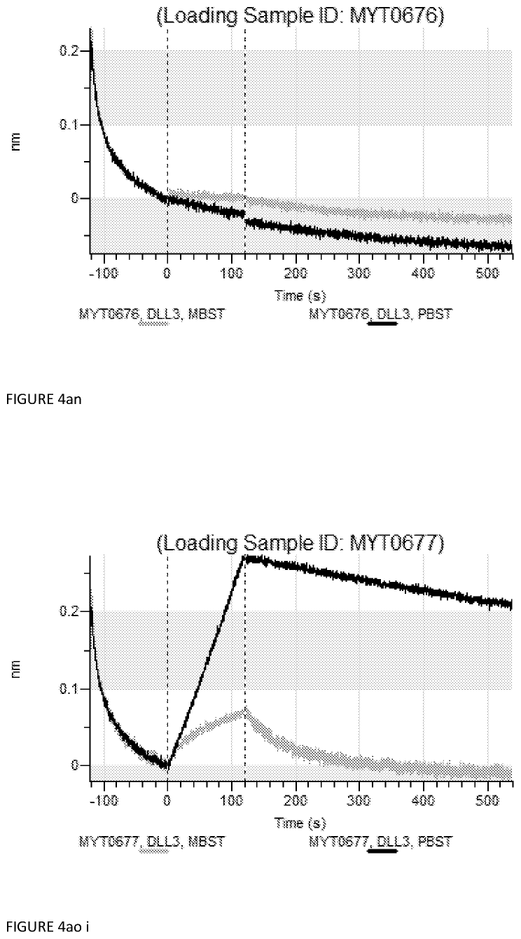

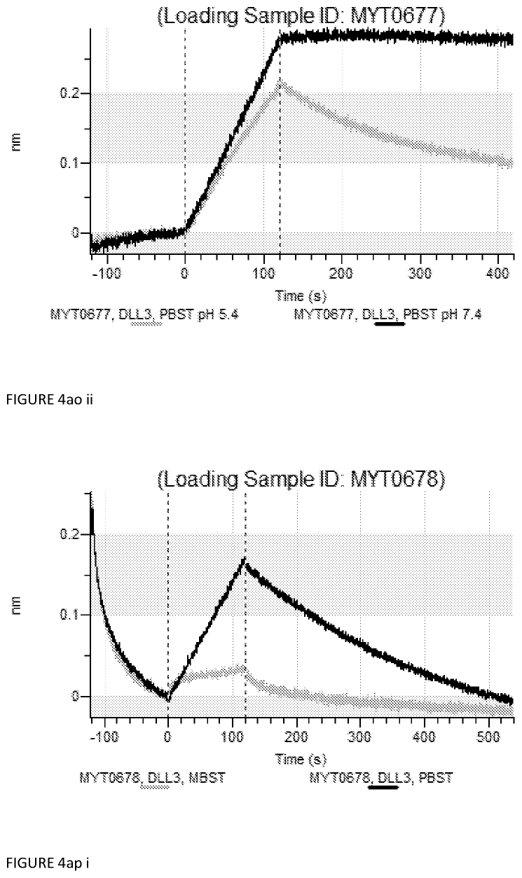

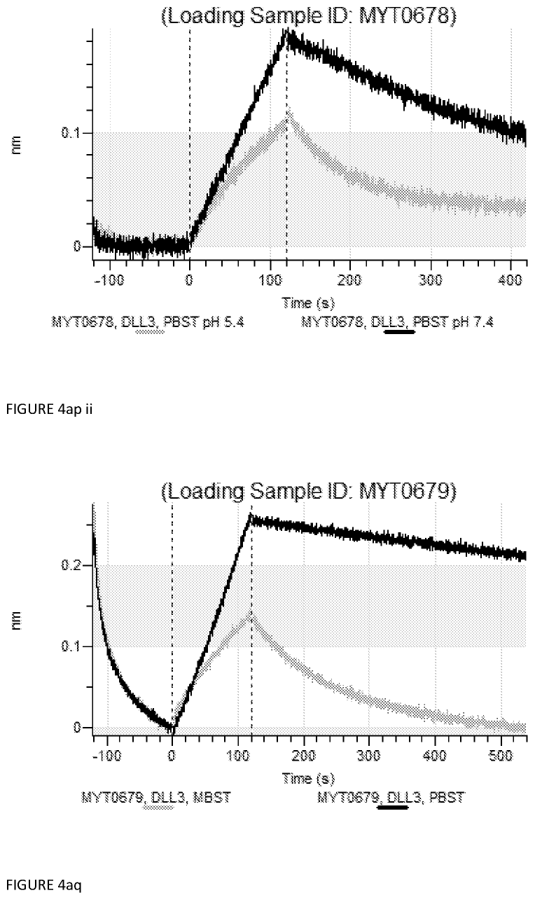

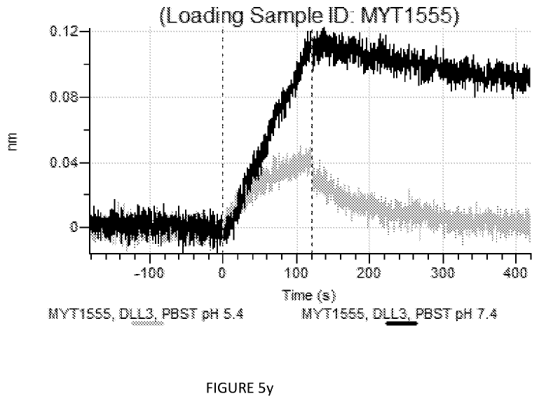

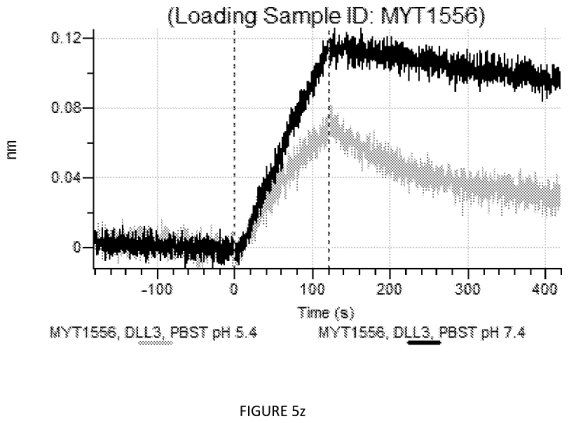

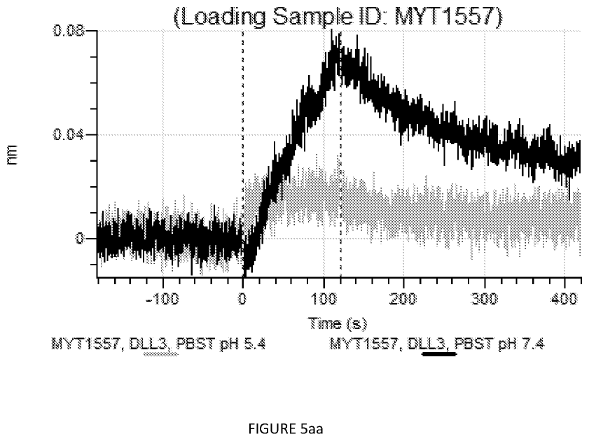

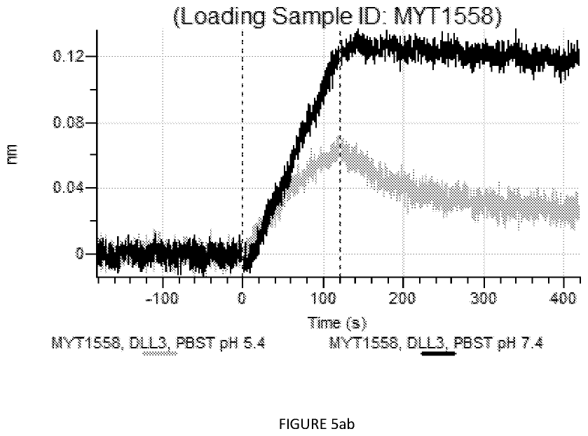

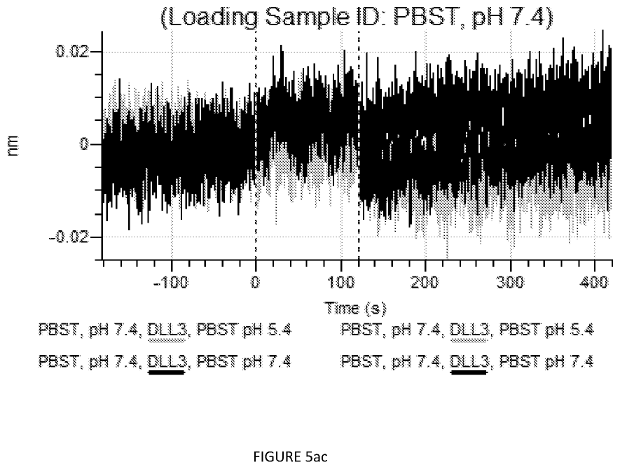

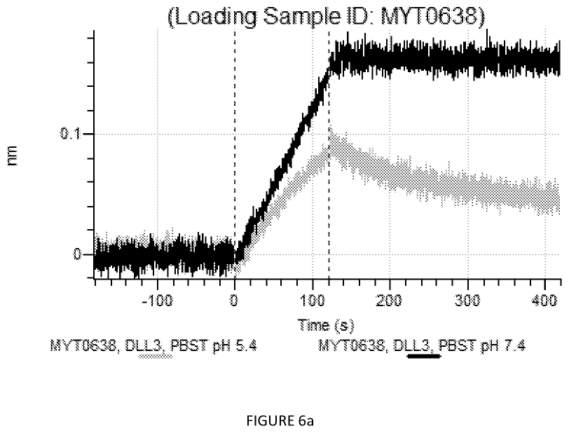

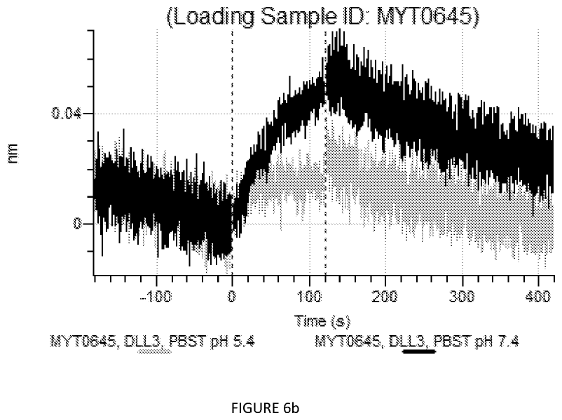

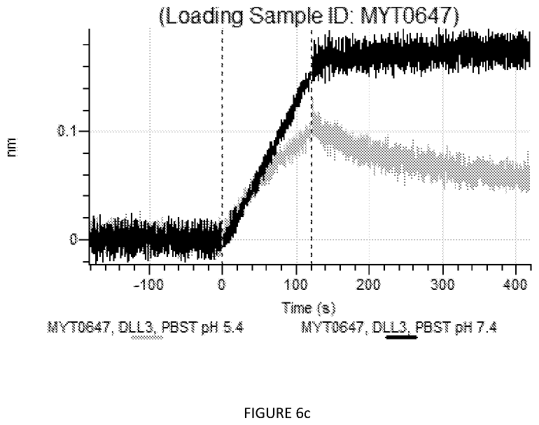

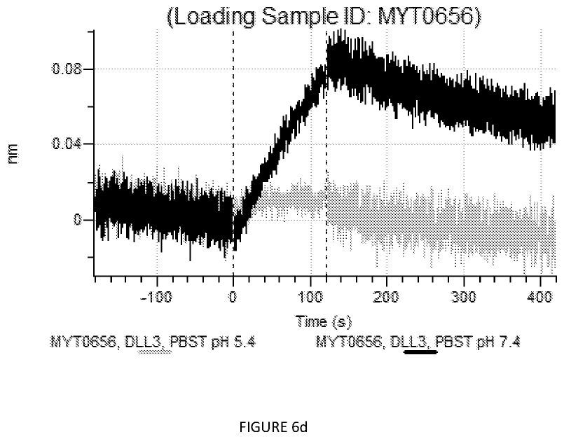

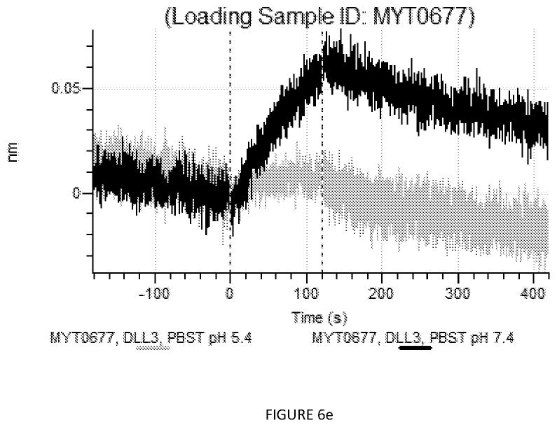

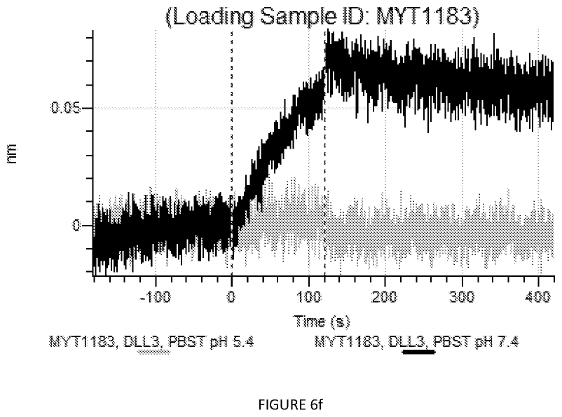

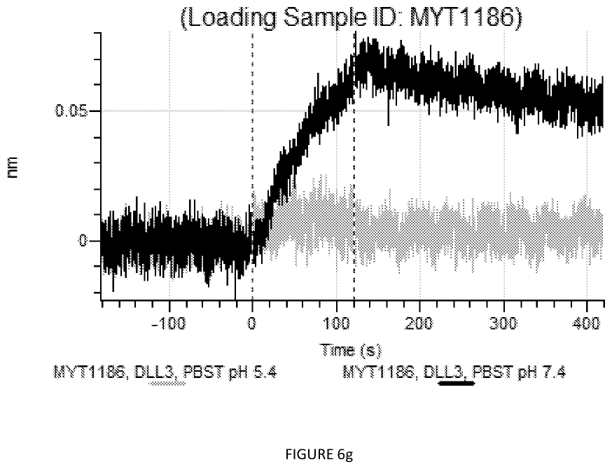

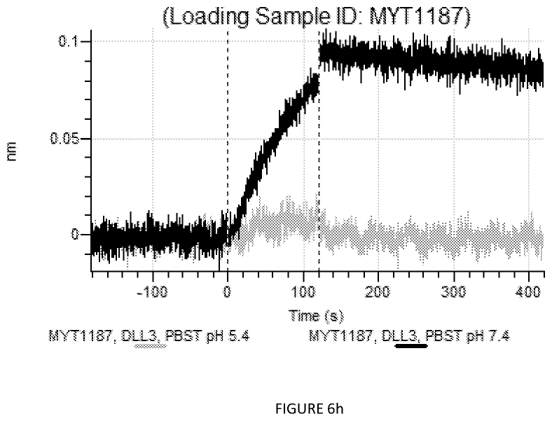

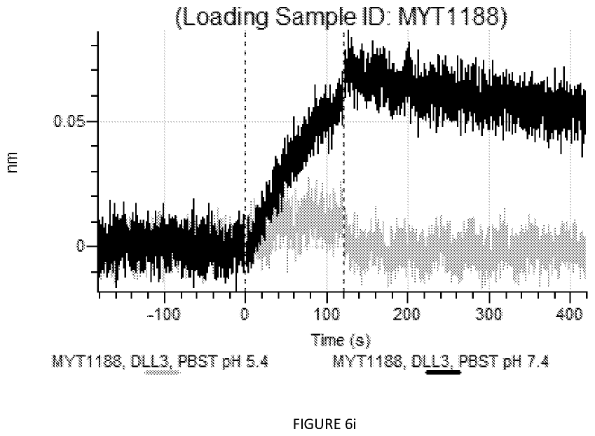

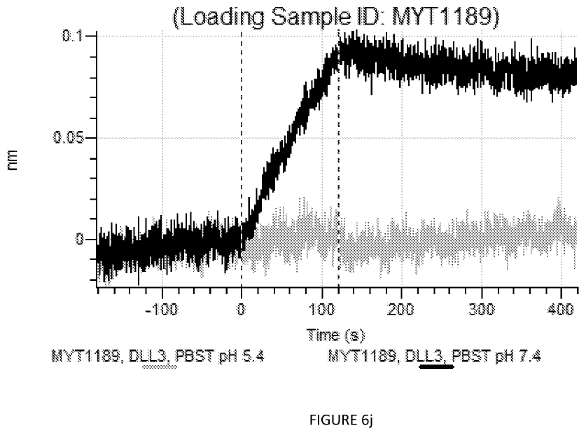

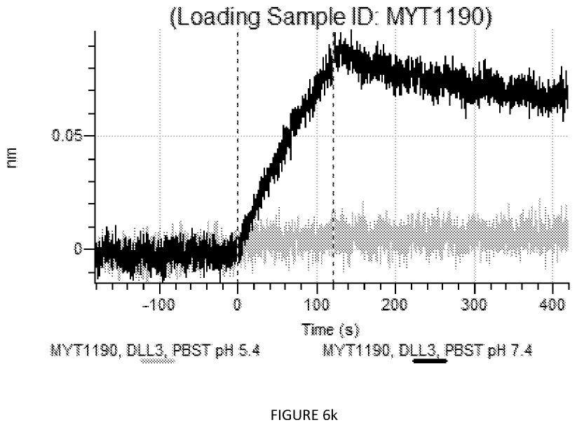

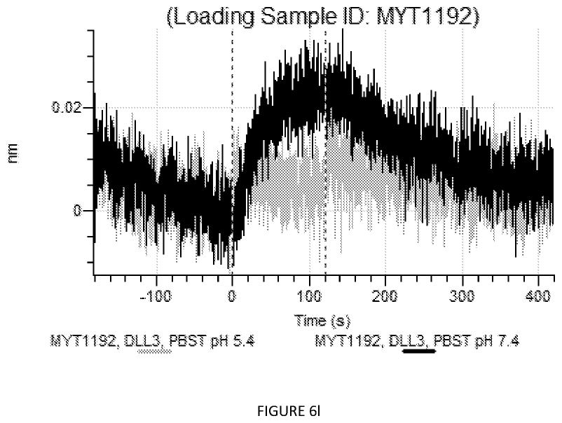

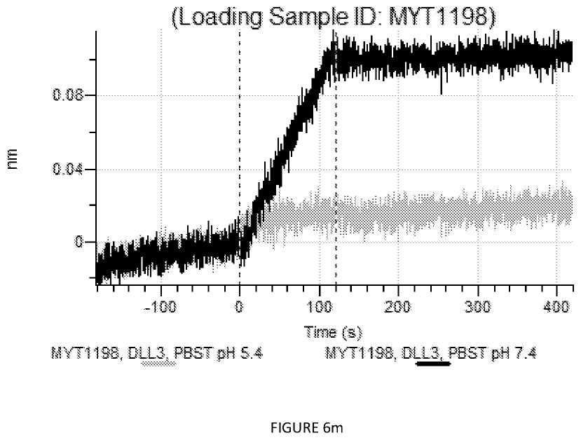

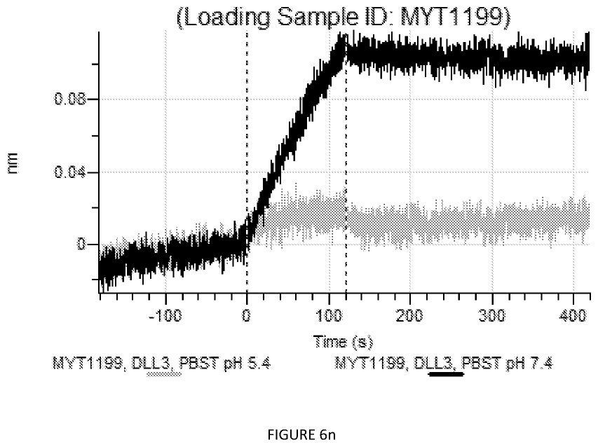

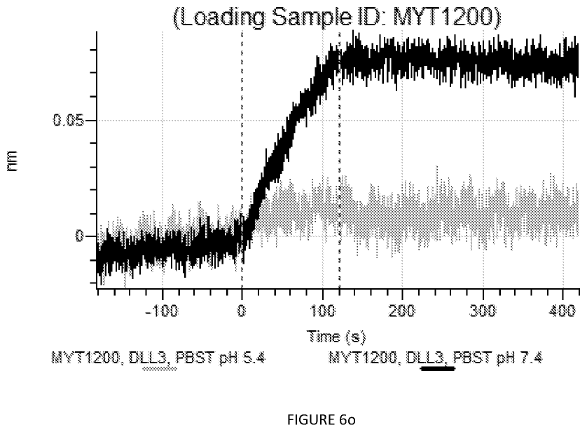

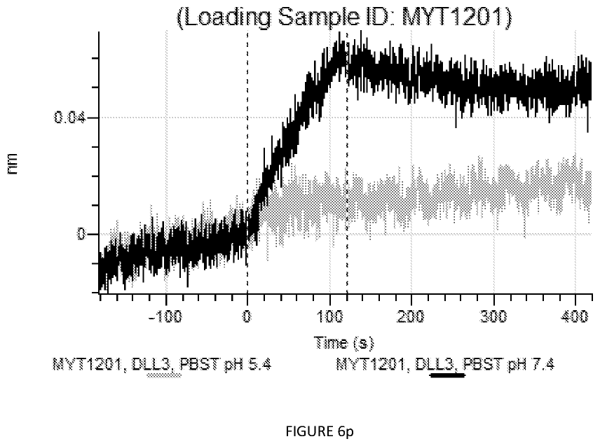

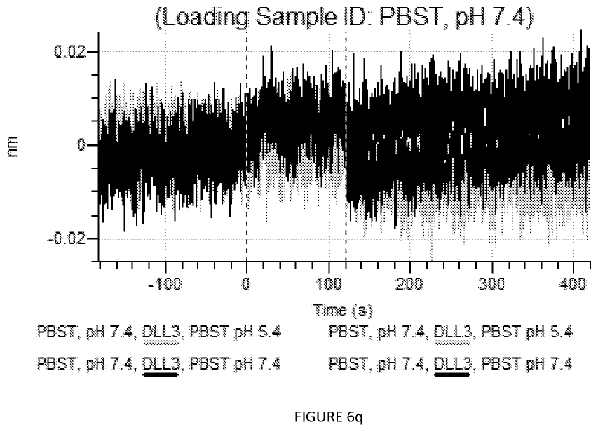

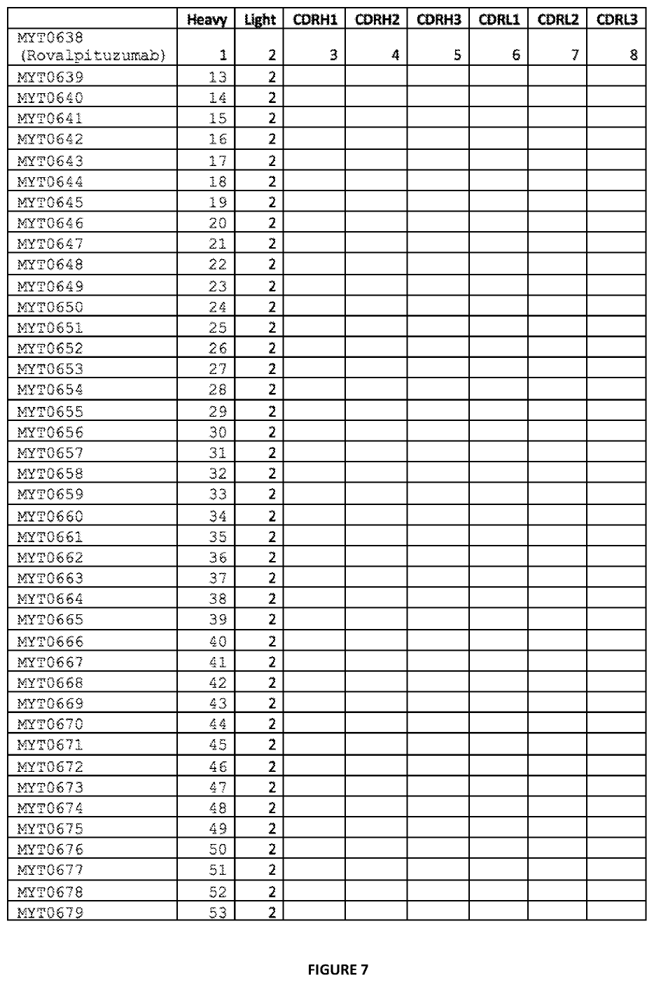

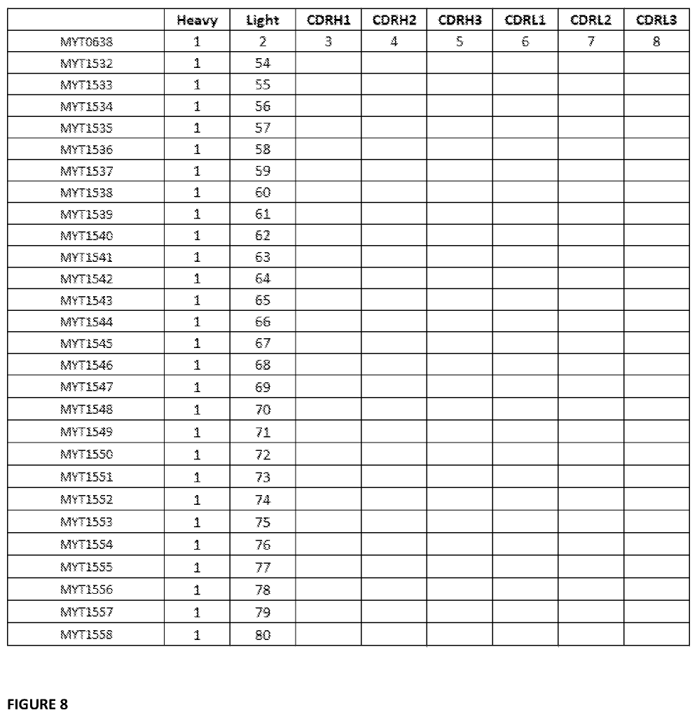

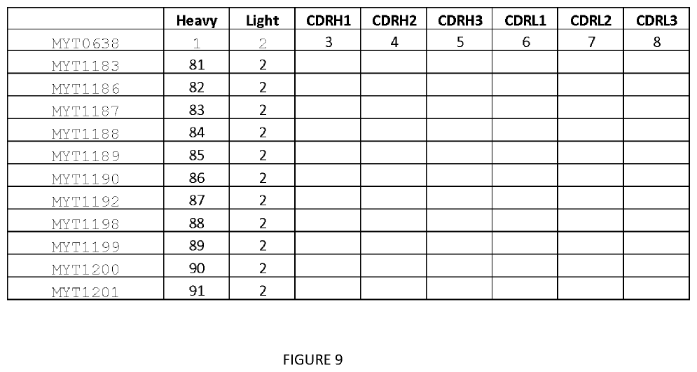

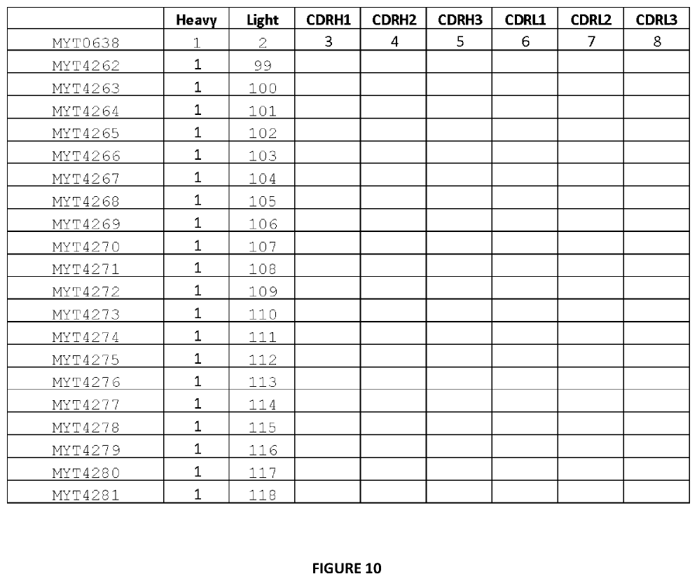

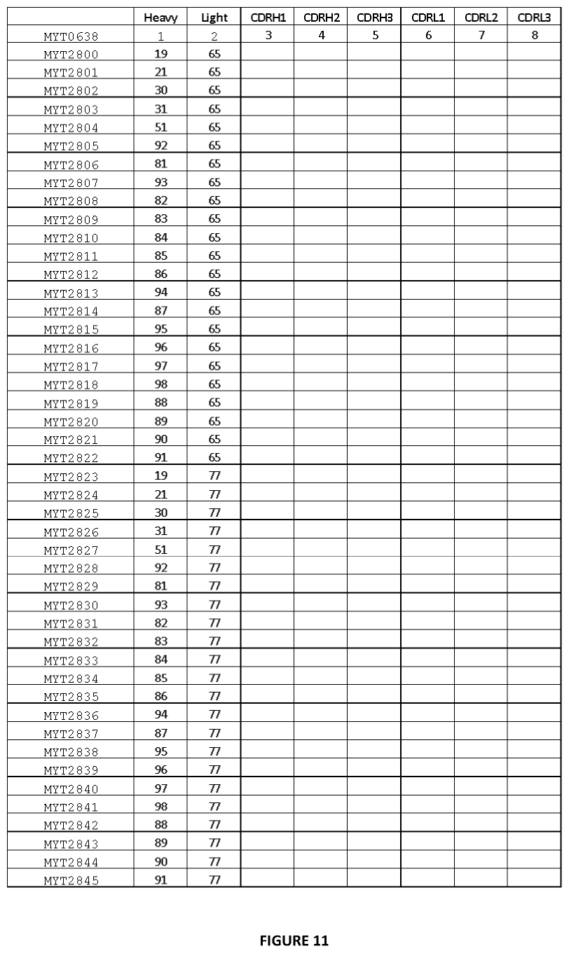

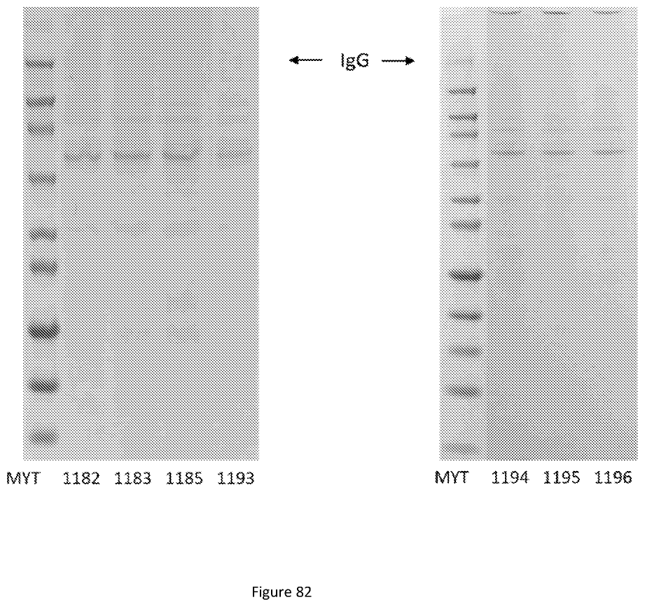

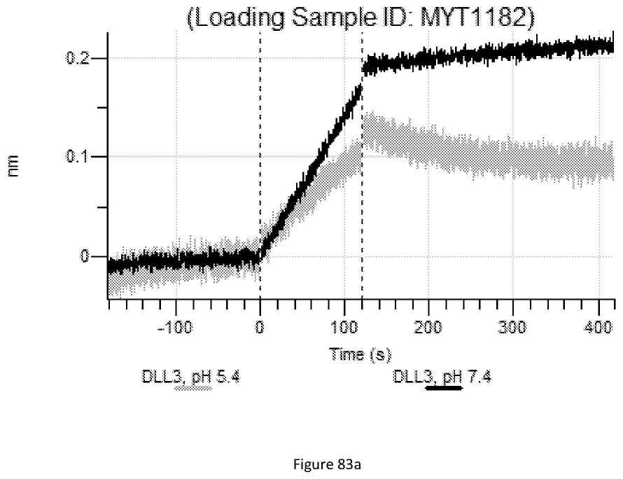

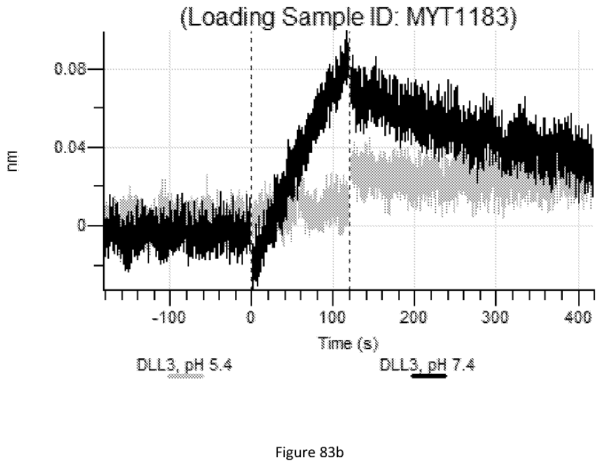

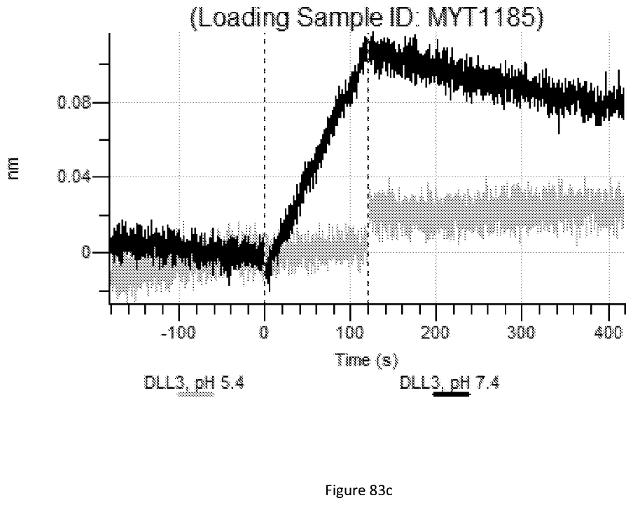

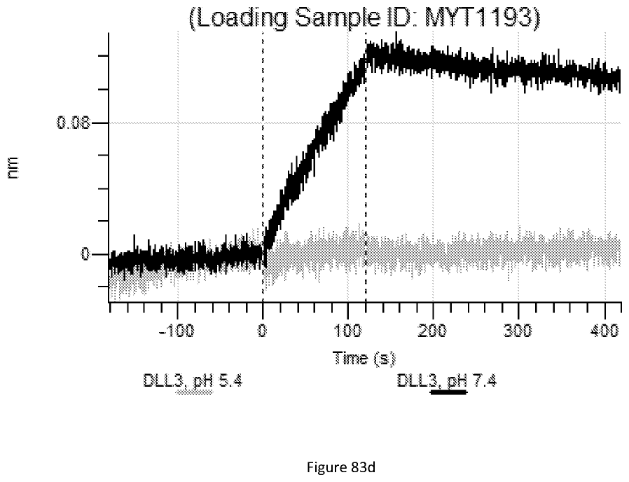

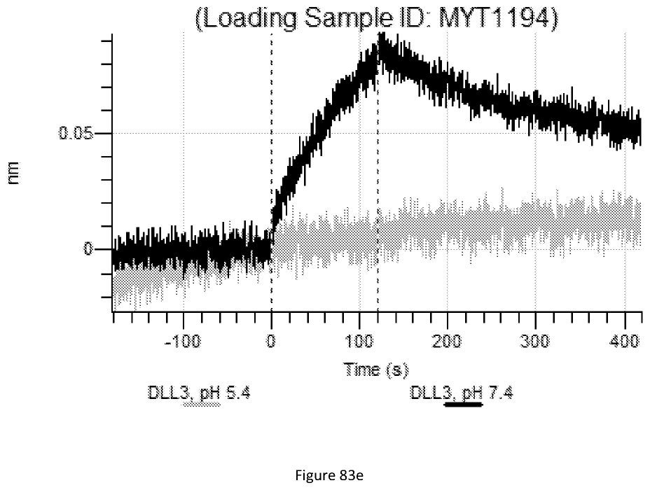

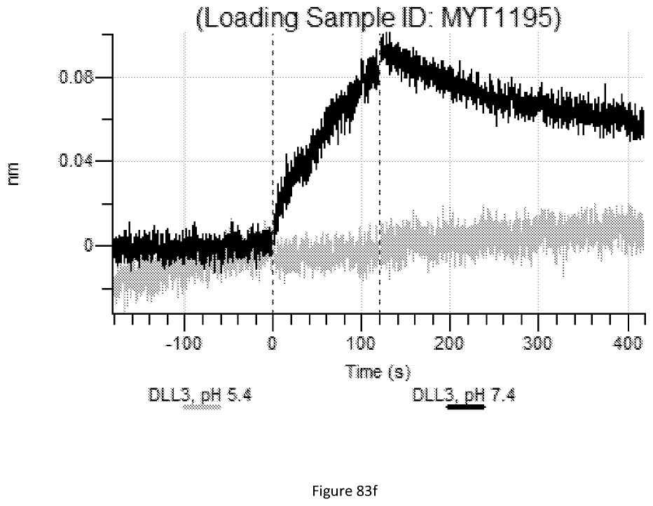

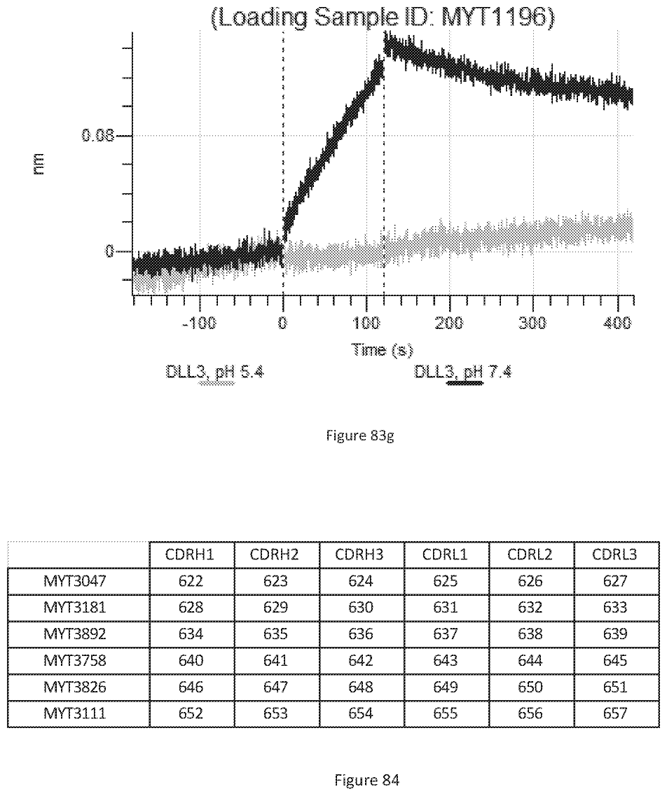

Multiple DLL3-binding monoclonal antibodies have been described in the literature and can be used as a template for engineering pH-dependent binding [Saunders L R (2016) “A DLL3-targeted antibody-drug conjugate eradicates high-grade pulmonary neuroendocrine tumor-initiating cells in vivo” Science Translational Medicine 7(302):302ra136]. ROVALPITUZUMAB (Heavy chain SEQ ID NO: 1, Light chain SEQ ID NO: 2) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy chain CDRs that had been previously selected for further analysis in Example 9 were systematically combined two or more at a time (MYT1182, MYT1183, MYT1185, MYT1186, MYT1187, MYT1188, MYT1189, MYT1190, MYT1192, MYT1193, MYT1194, MYT1195, MYT1196, MYT1198, MYT1199, MYT1200, MYT1201). In cases where the starting CDR residue was a histidine, it was mutated to an alanine. Antibody variants with two or more histidine or alanine mutations in the heavy chain CDRs were generated by co-transfection of Expi293 cells with a) one heavy chain combinations sequence variant, and b) the corresponding starting ABPC light chain using methods known to the art. After allowing for four days of protein expression, cell culture supernatants were collected, quantified by SDS-PAGE analysis (FIGS. 3 and 82), and the pH dependence of the variant was evaluated using biolayer interferometry (BLI) on an Octet RED 384 instrument. Briefly, 5 μL of cell culture supernatant was diluted into 195 μL of 1×PBST pH 7.4 for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20) pH 7.4, and the sensor was associated with 50 nM of DLL3 (R&D Systems, Cat #9749-DL, Lot No. DFSG0719011) in 1×PBST pH 7.4 for 120 sec to generate an association curve. In the dissociation phase, the antibody-antigen complex on the sensor was exposed to 1×PBST pH 7.4 for 300-600 sec. Baseline, association, and dissociation were repeated using 1×PBST pH 5.4 throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain combinations variants that showed either enhanced dissociation at pH 5.4 or reduced dissociation at pH 7.4 or both (as compared to the starting ABPC), as shown in FIGS. 6 and 83 were selected for further analysis (e.g., MYT1182, MYT1183, MYT1185, MYT1186, MYT1187, MYT1188, MYT1189, MYT1190, MYT1192, MYT1193, MYT1194, MYT1195, MYT1196, MYT1198, MYT1199, MYT1200, MYT1201).

### Example 12. Construction and Screening of pH-Engineered DLL3 ABPCs

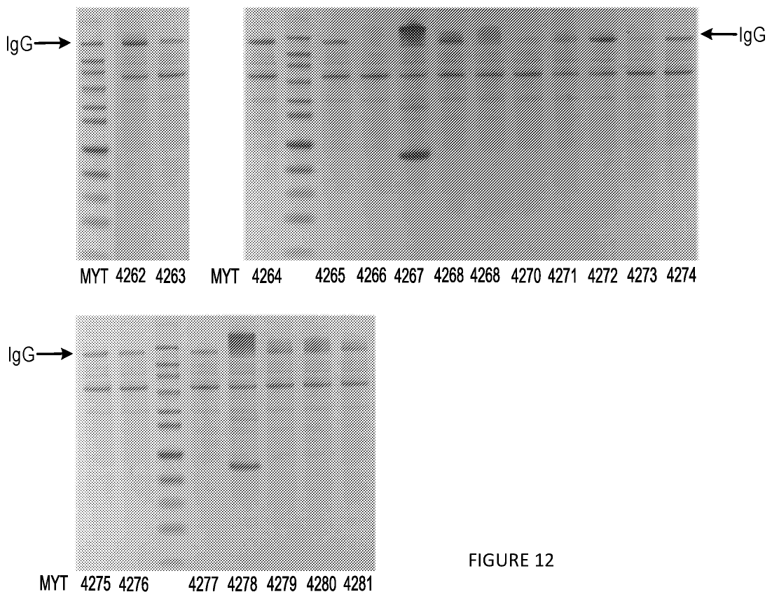

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). ROVALPITUZUMAB (Heavy chain SEQ ID NO: 1, Light chain SEQ ID NO: 2) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the light chain CDRs that had been previously selected for further analysis in Example 10 were systematically combined two or more at a time (MYT4262-4281). In cases where the starting CDR residue was a histidine, it was mutated to an alanine. Antibody variants with two or more histidine or alanine mutations in the light chain CDRs were generated by co-transfection of Expi293 cells with a) one light chain combinations sequence variant, and b) the corresponding starting ABPC heavy chain using methods known to the art. After allowing for four days of protein expression, cell culture supernatants were collected, quantified by SDS-PAGE analysis (FIG. 12), and the pH dependence of the variant was evaluated using biolayer interferometry (BLI) on an Octet RED 384 or an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1× PBST, pH 7.4, for 120 sec to generate an association curve. In the dissociation phase, the antibody-antigen complex on the sensor was exposed to 1×PBST, pH 7.4, for 300-600 sec.

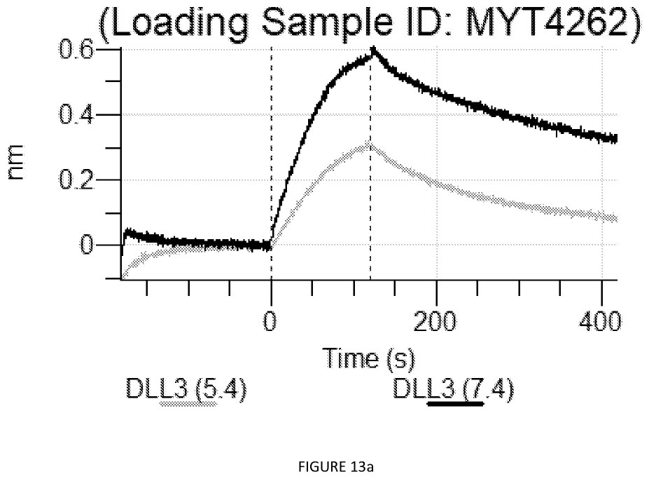

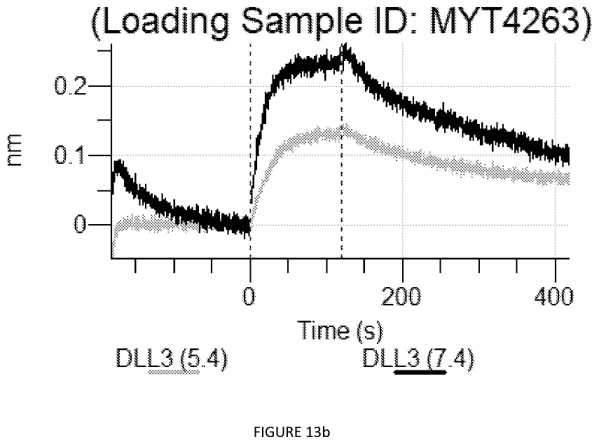

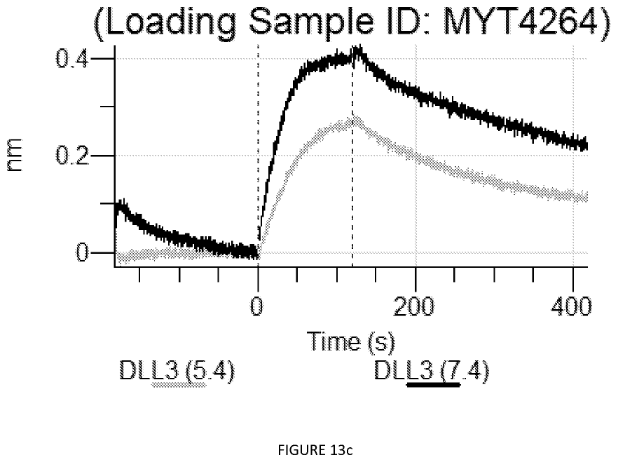

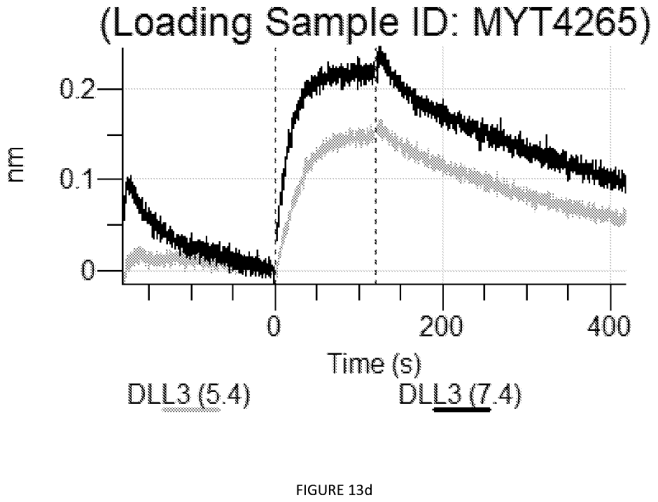

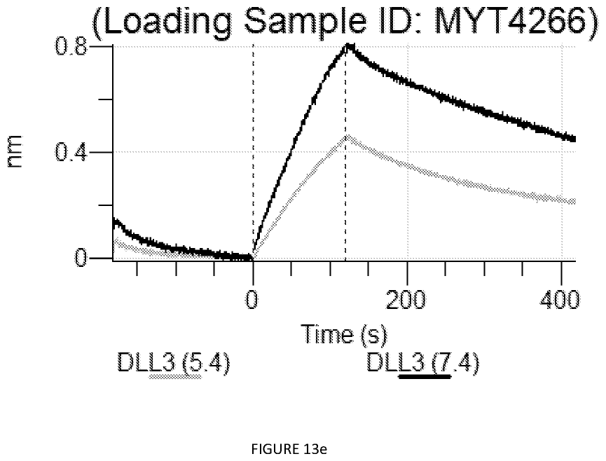

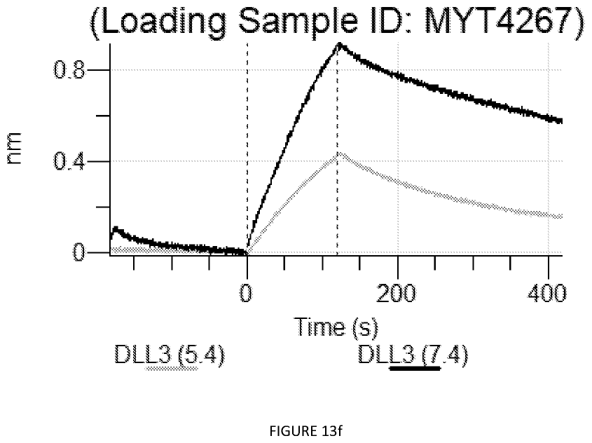

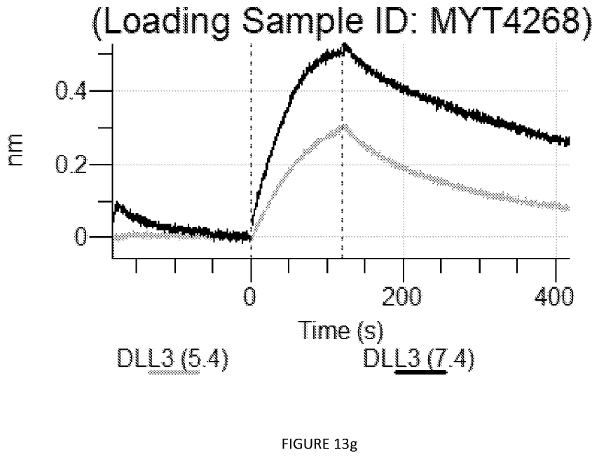

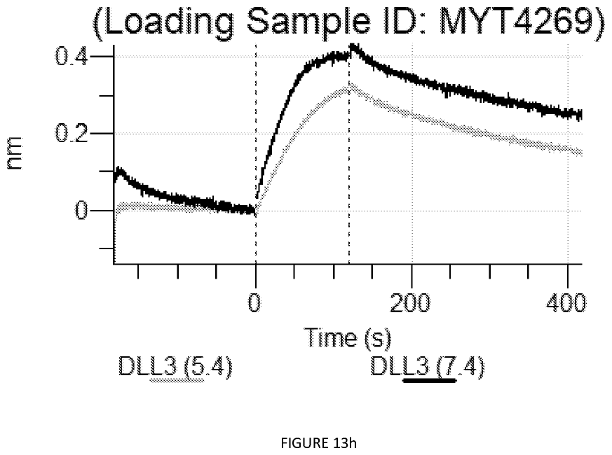

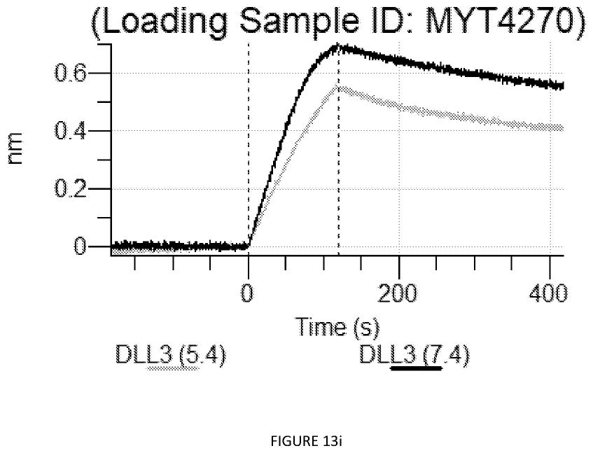

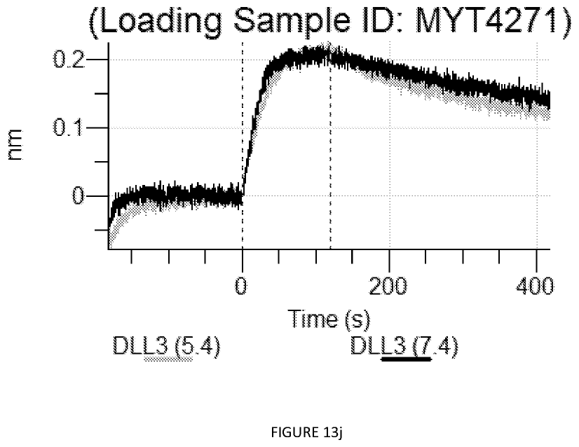

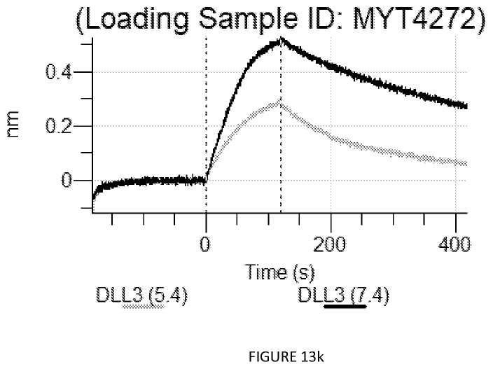

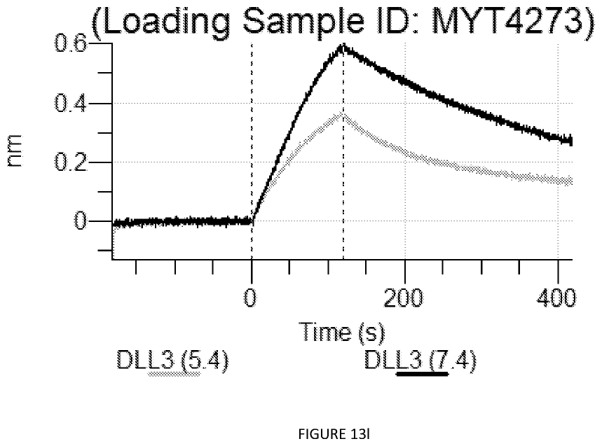

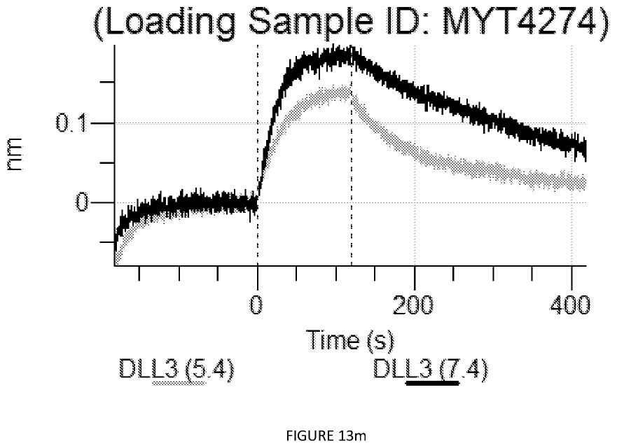

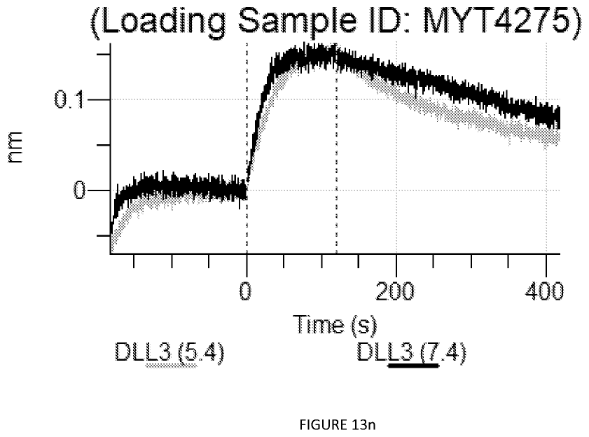

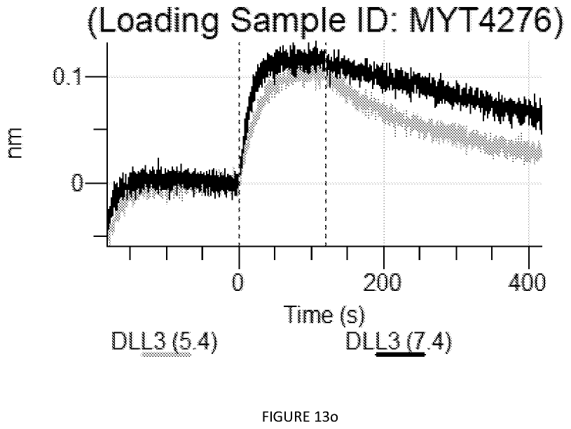

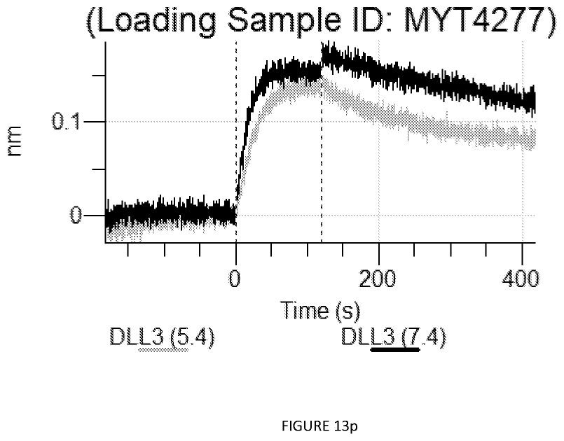

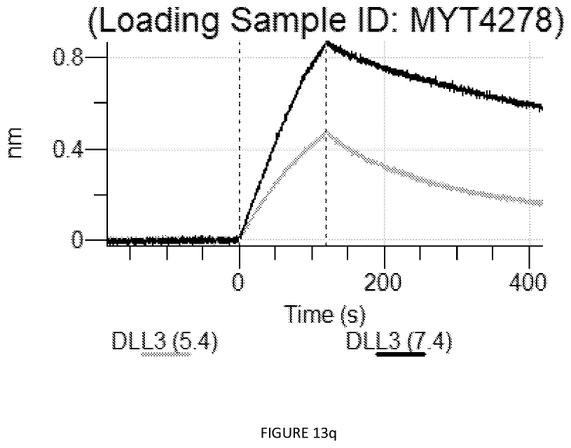

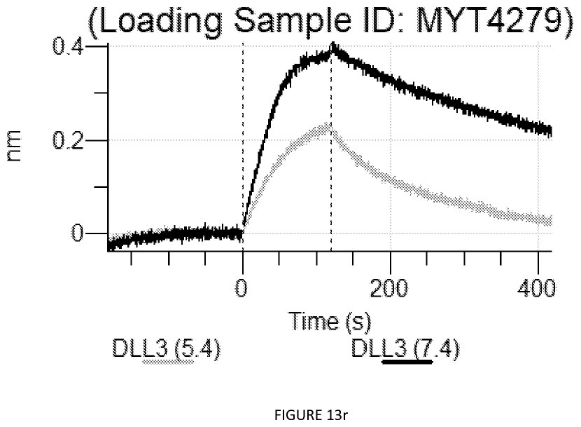

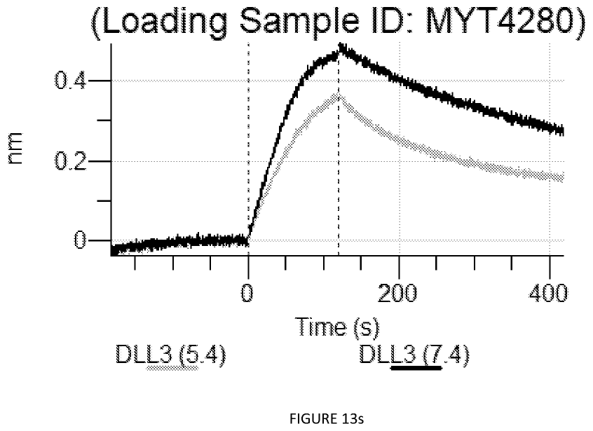

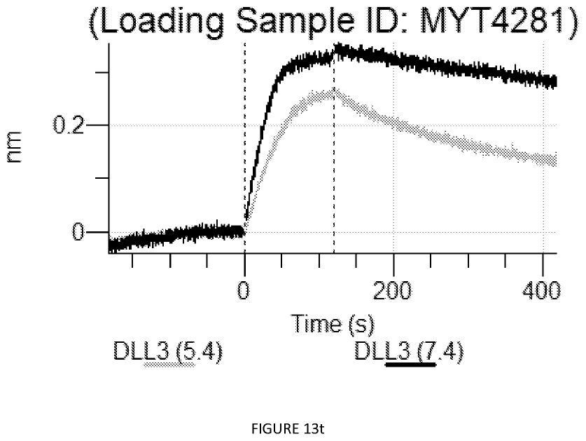

Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain combinations variants that showed either enhanced dissociation at pH 5.4 or reduced dissociation at pH 7.4 or both (as compared to the starting ABPC), as shown in FIG. 13 were selected for further analysis (MYT4262-4281).

### Example 13. Construction and Screening of pH-Engineered DLL3 ABPCs

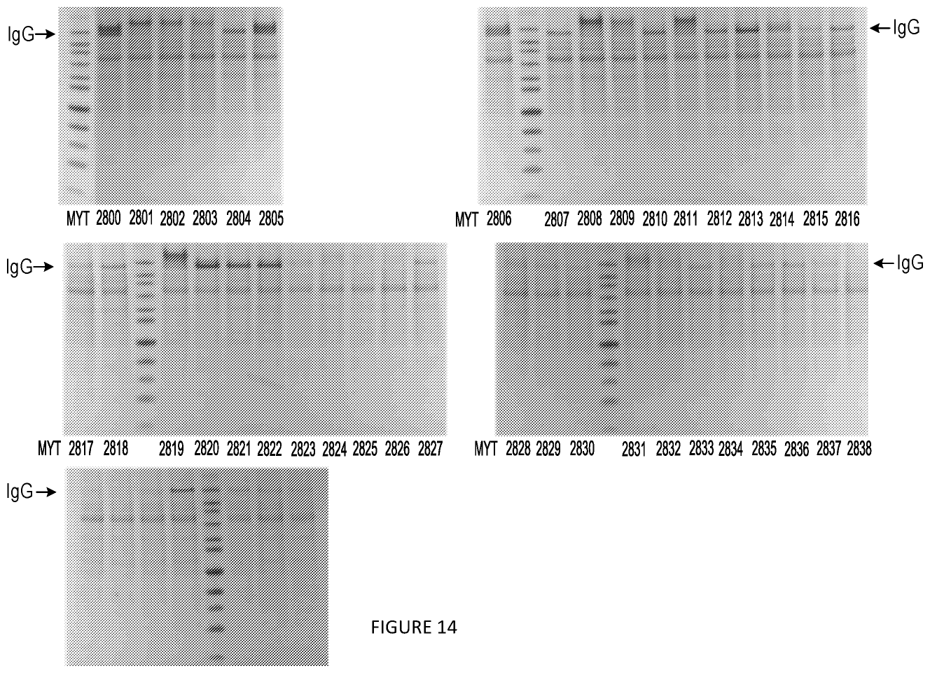

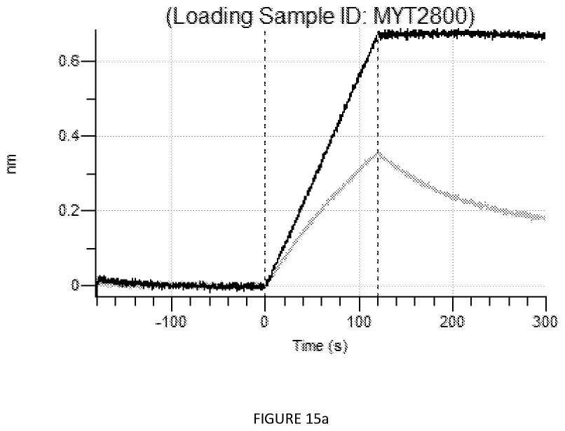

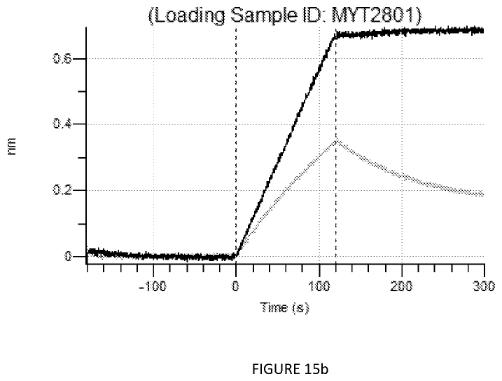

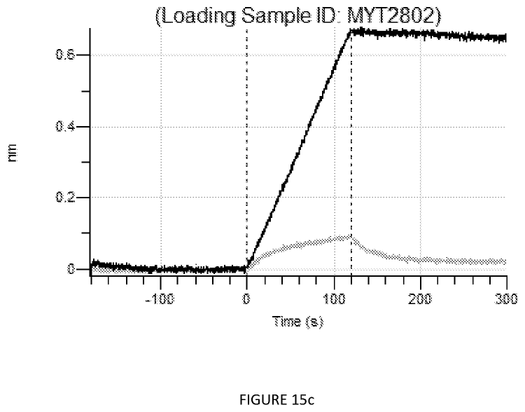

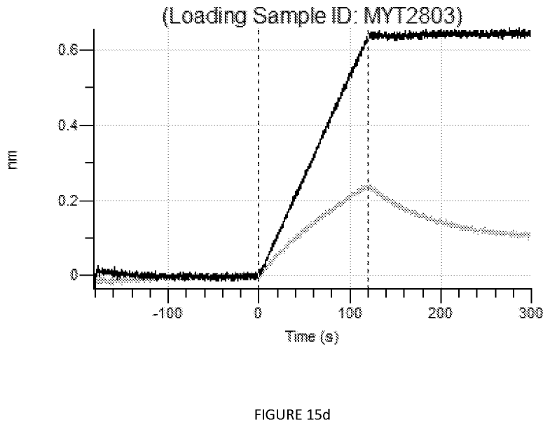

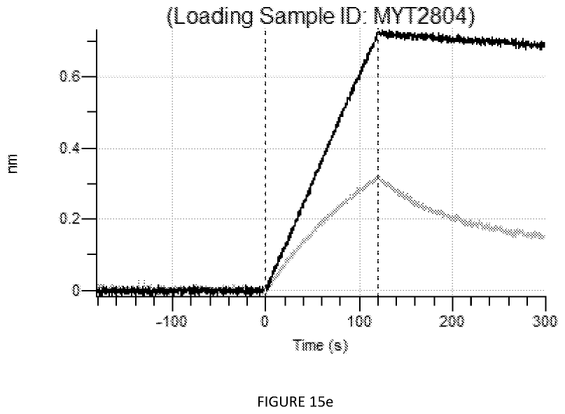

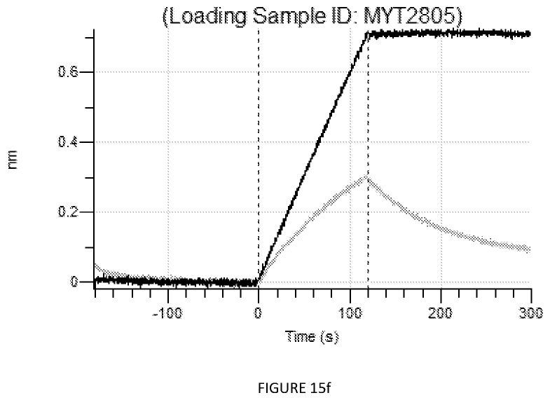

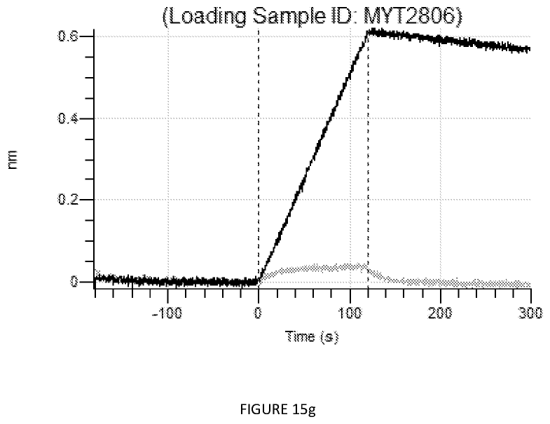

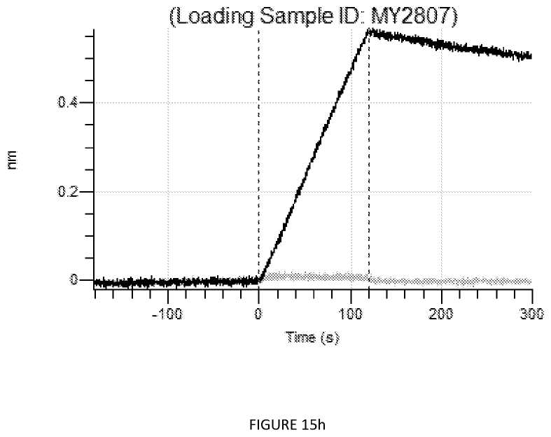

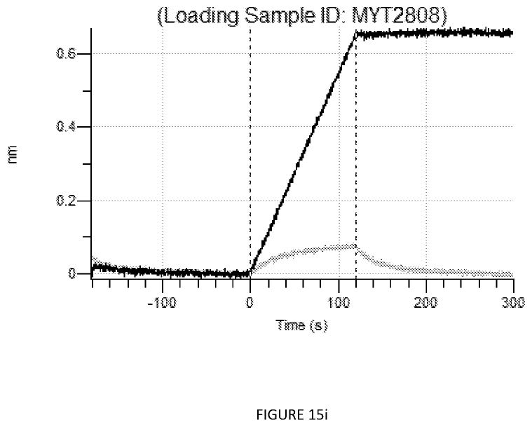

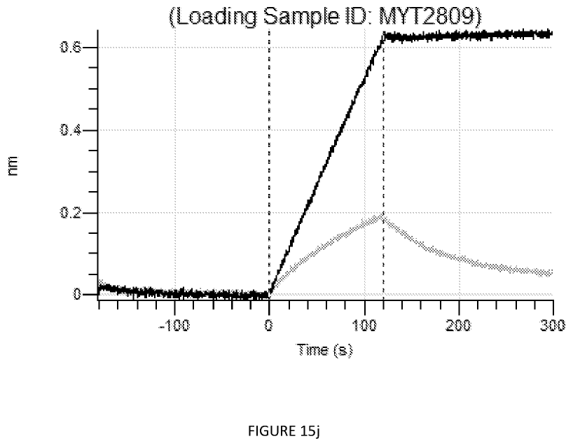

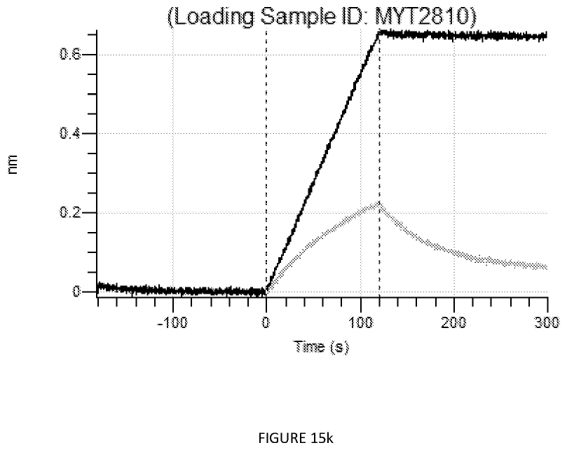

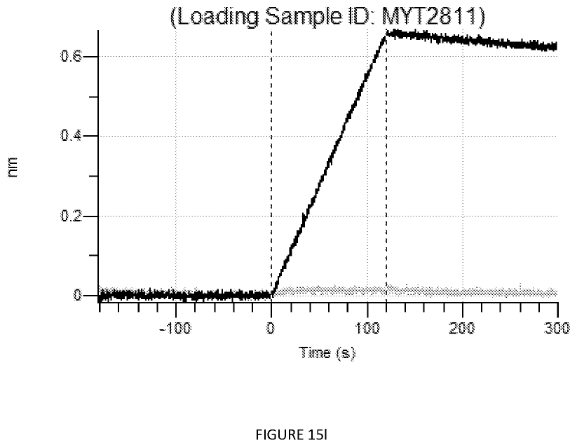

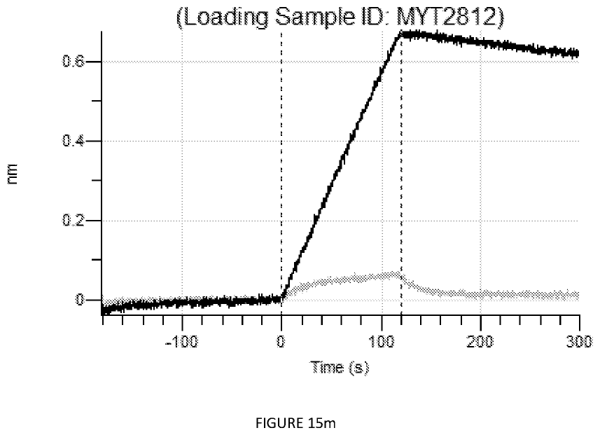

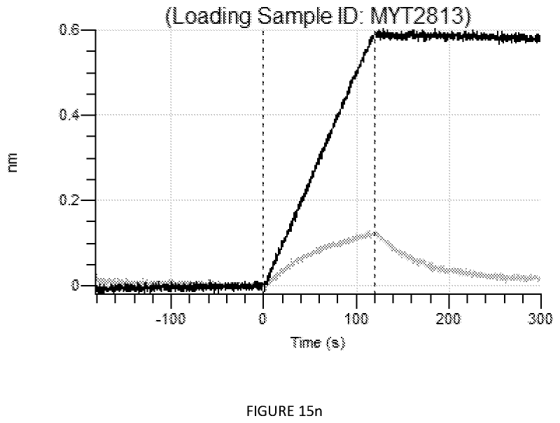

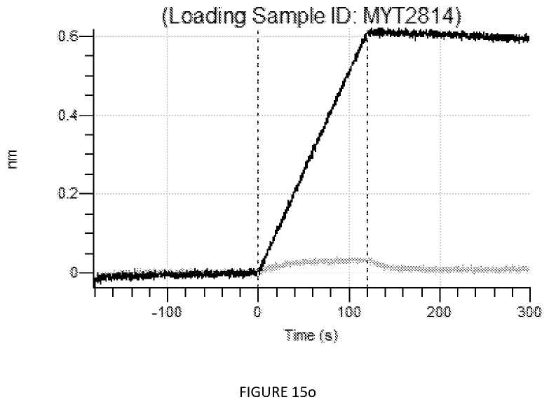

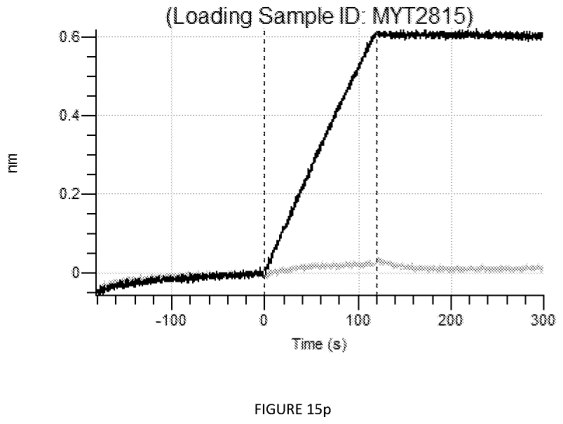

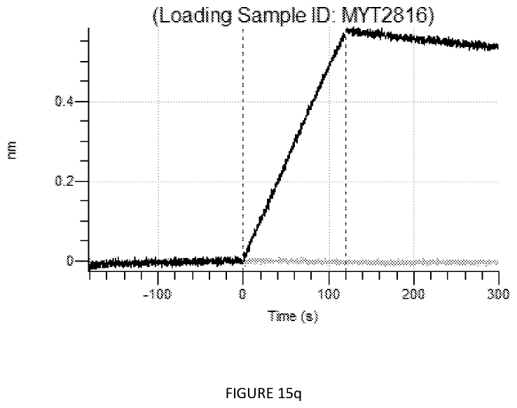

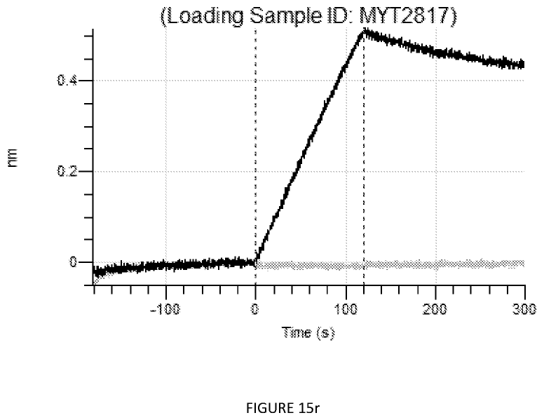

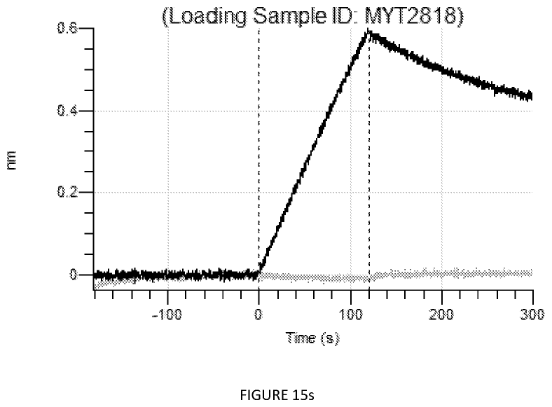

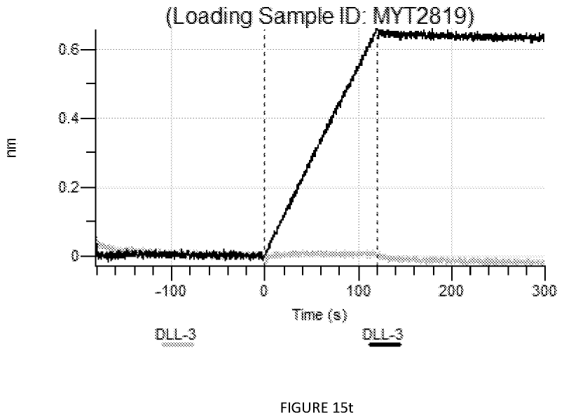

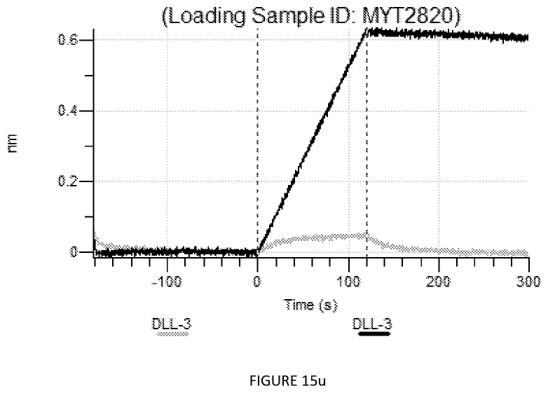

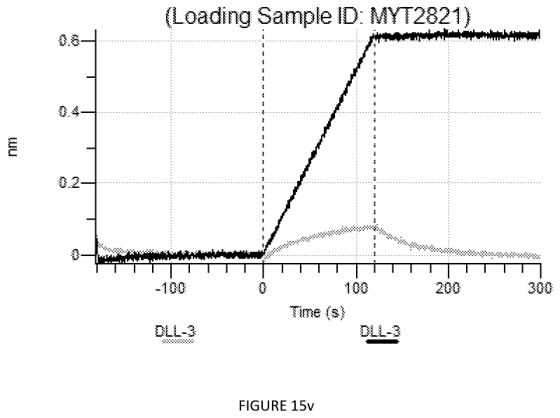

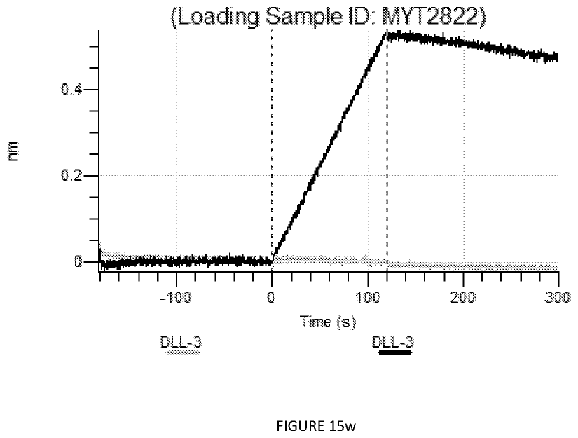

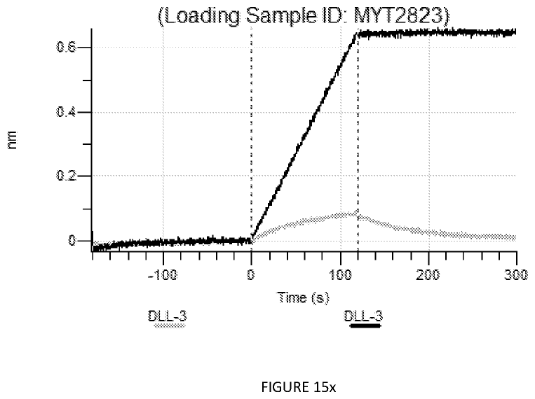

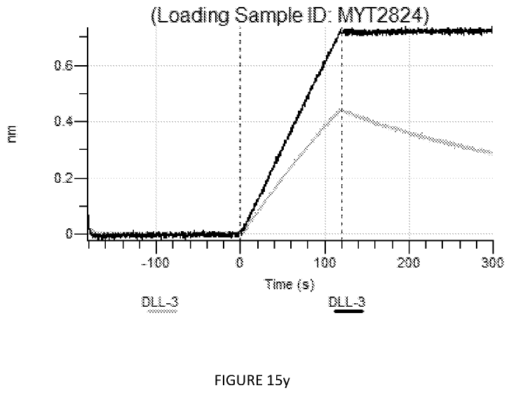

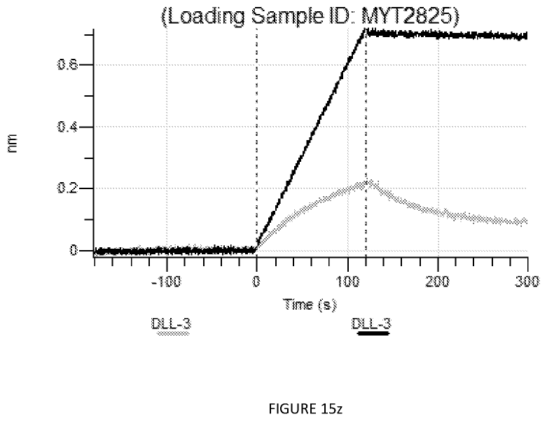

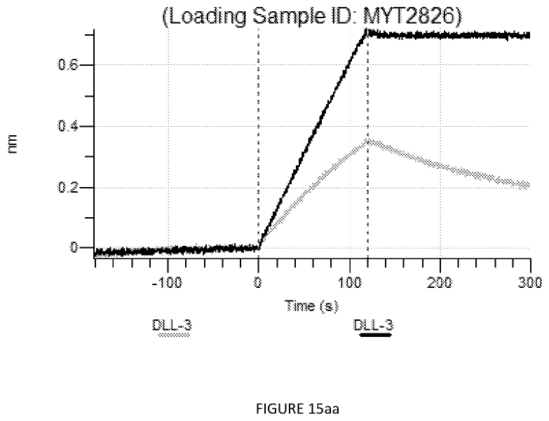

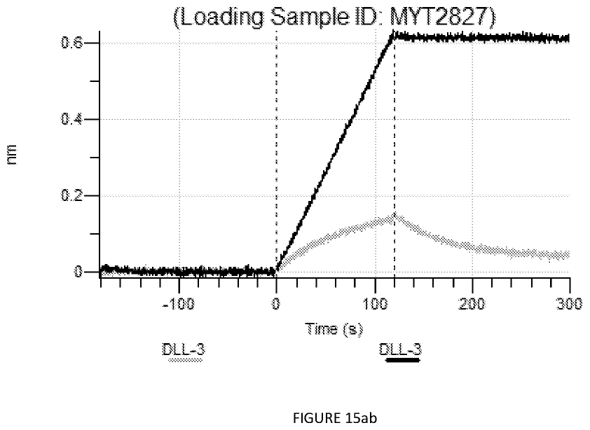

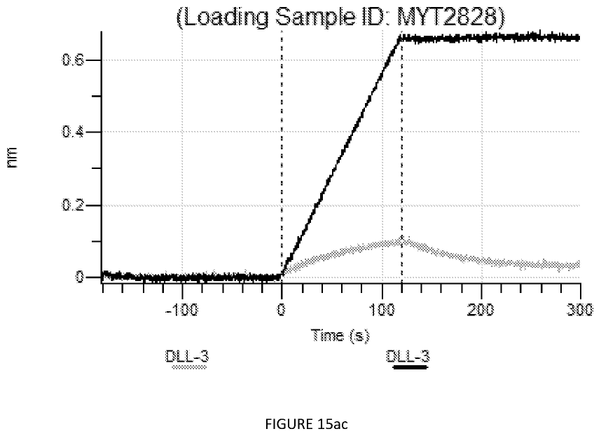

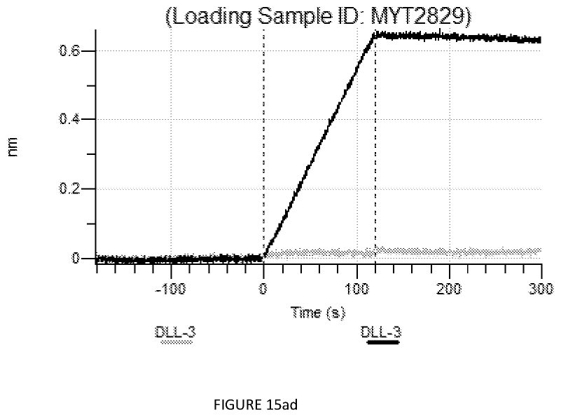

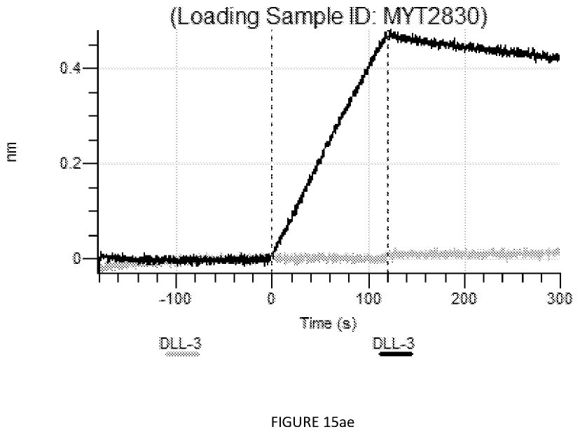

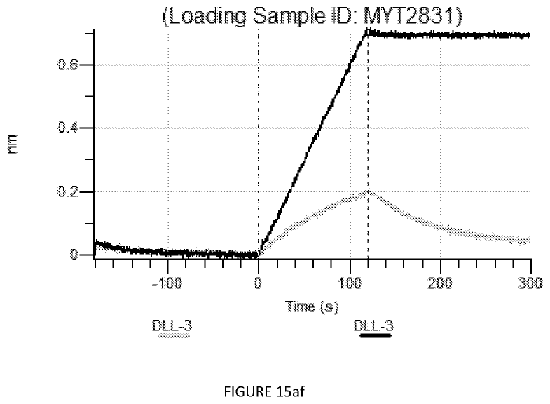

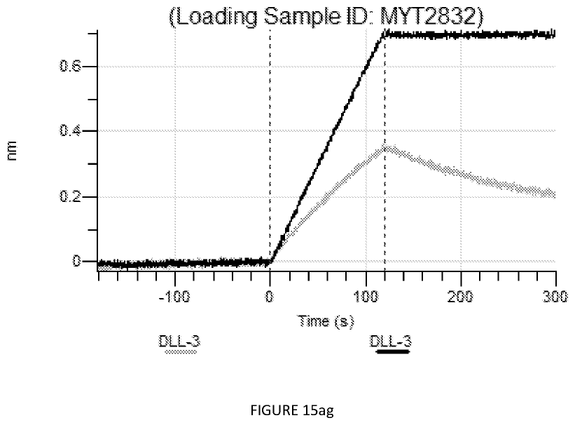

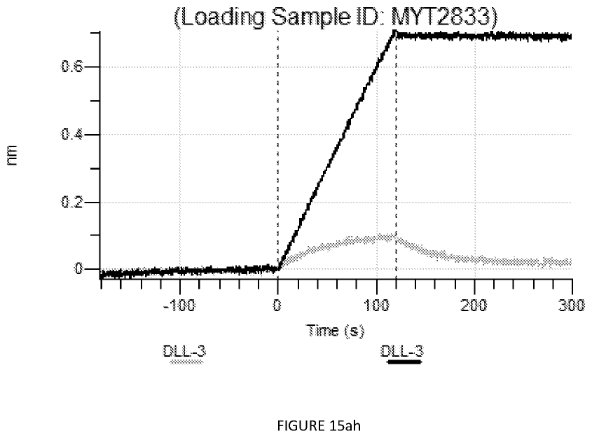

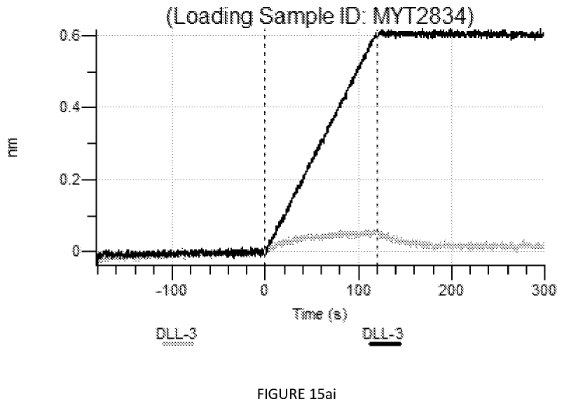

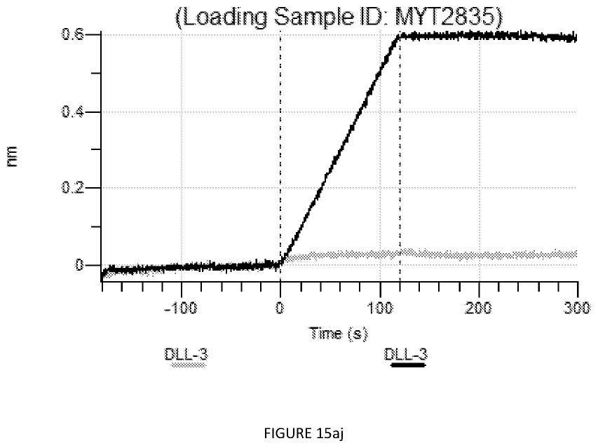

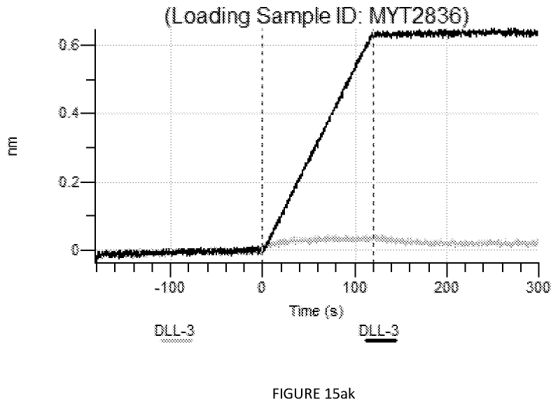

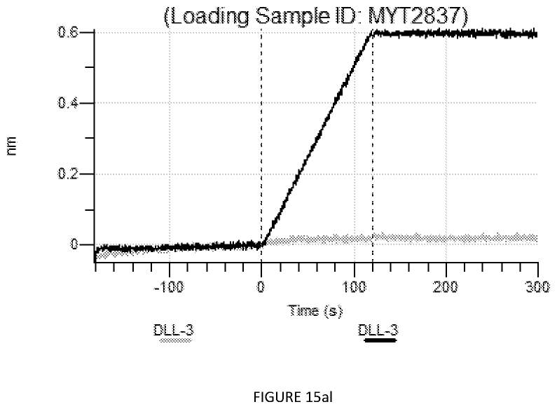

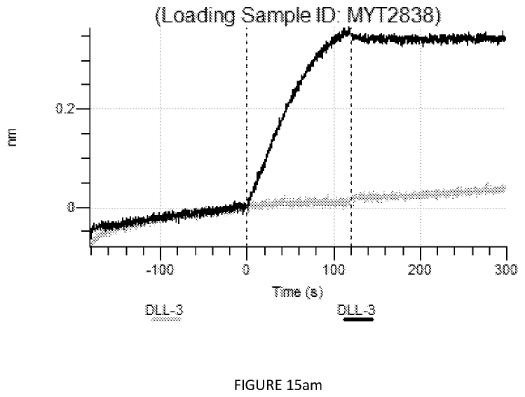

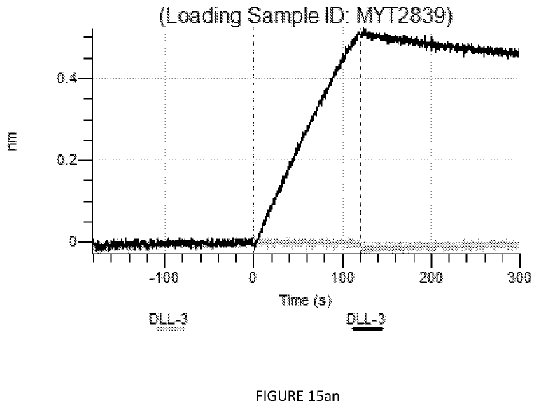

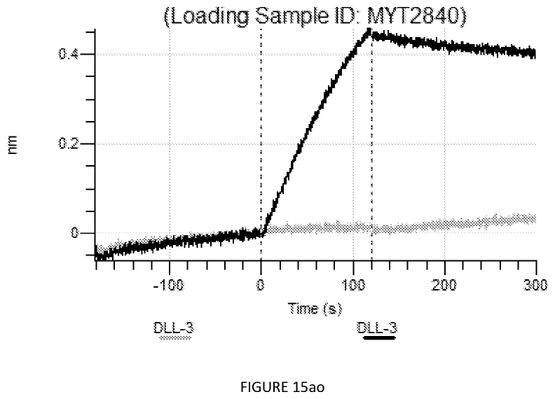

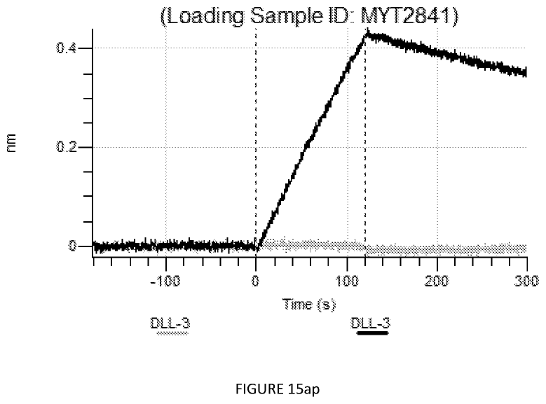

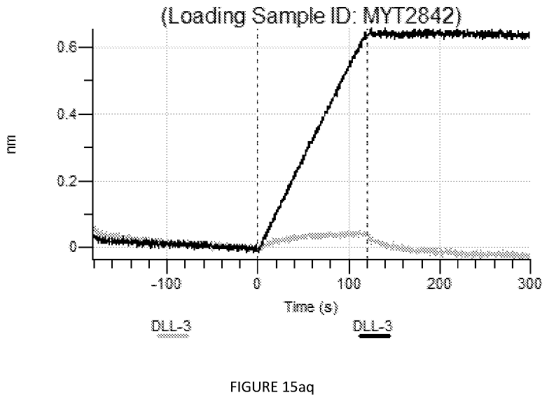

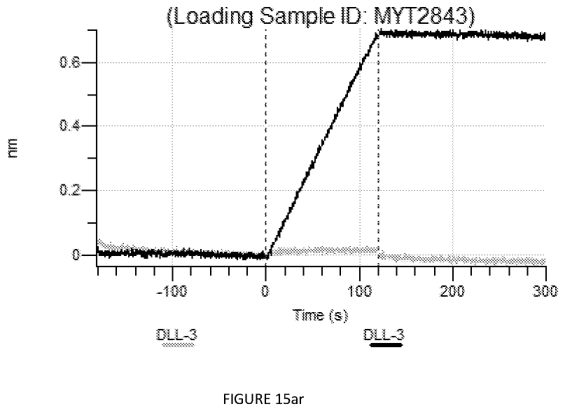

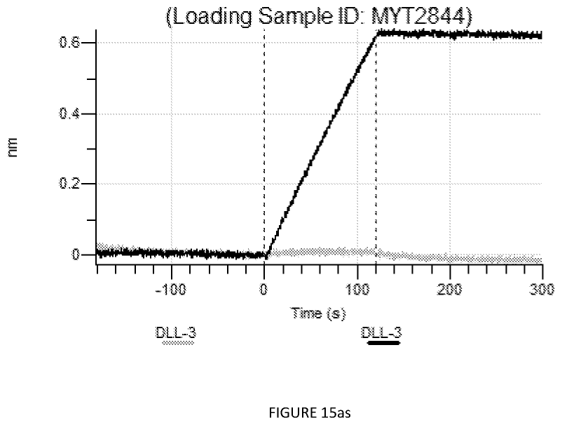

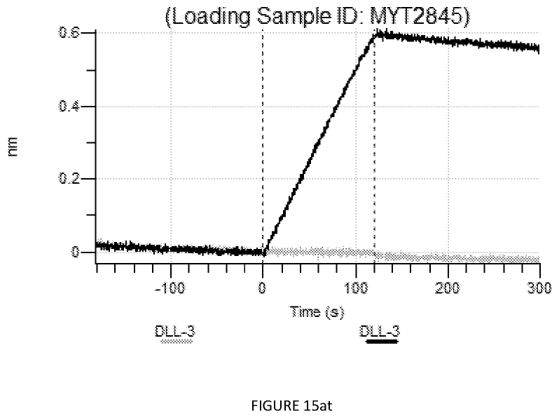

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). ROVALPITUZUMAB (Heavy chain SEQ ID NO: 1, Light chain SEQ ID NO: 2) was selected as a DLL3-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy and light chain CDRs that had been previously selected for further analysis in Examples 9-12 were systematically combined two or more at a time (MYT2800-2845). In cases where the starting CDR residue was a histidine, it was mutated to an alanine. Antibody variants with two or more histidine or alanine mutations were generated by co-transfection of Expi293 cells with a) one light chain sequence variant, and b) one heavy chain sequence variant or heavy chain combinations sequence variant using methods known to the art. After allowing for four days of protein expression, cell culture supernatants were collected, quantified by SDS-PAGE analysis (FIG. 14), and the pH dependence of the variant was evaluated using biolayer interferometry (BLI) on an Octet RED 384 or an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Paired heavy and light chain variants that showed either enhanced dissociation at pH 5.4 or reduced dissociation at pH 7.4 or both (as compared to the starting ABPC), as shown in FIG. 15, were selected for further analysis (MYT2800-2845).

### Example 14. Construction and Screening of pH-Engineered DLL3 ABPCs

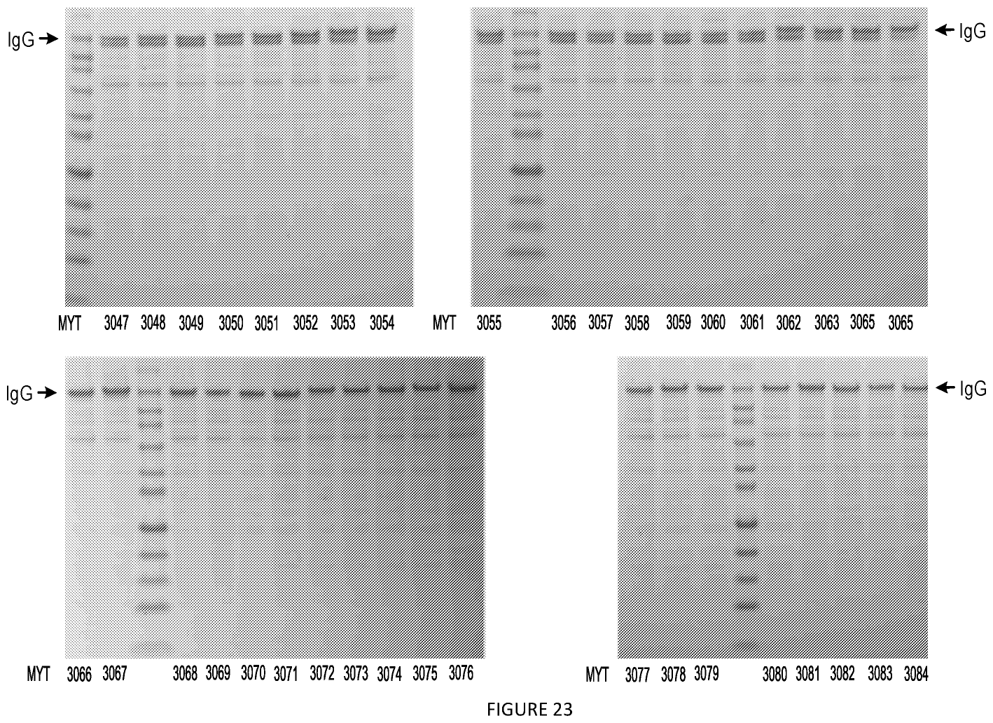

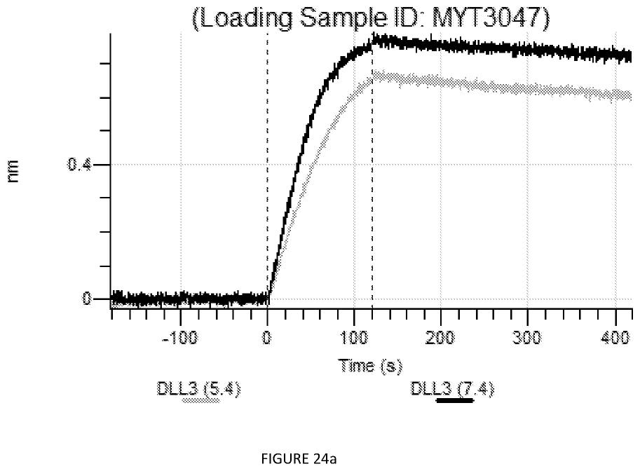

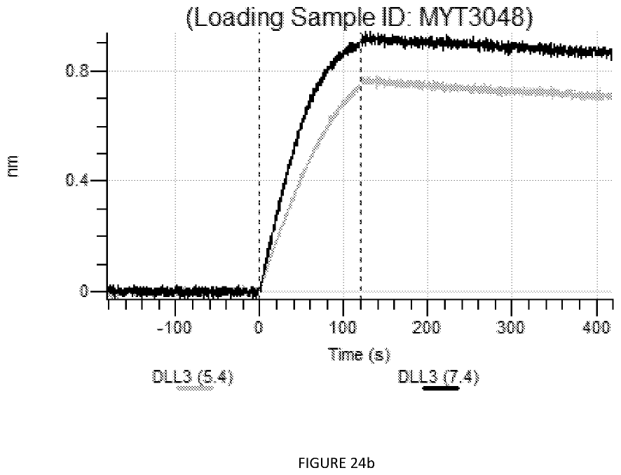

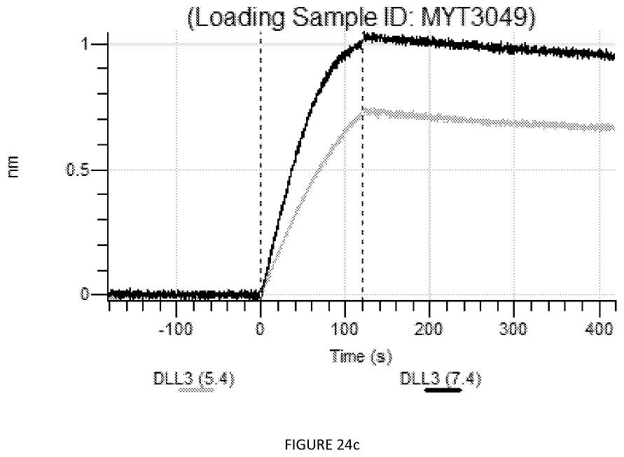

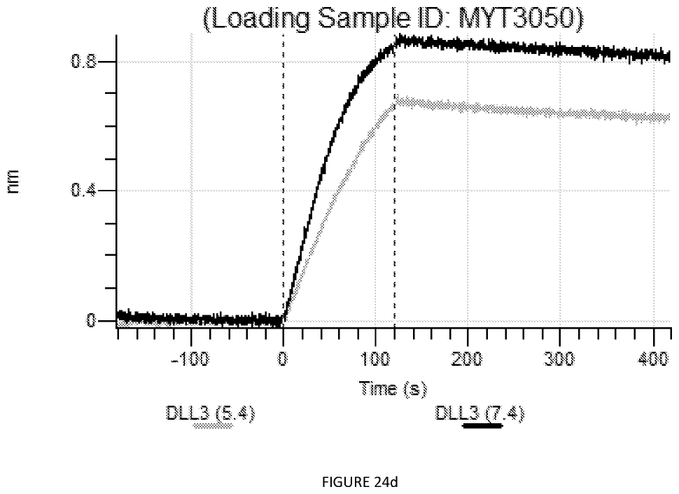

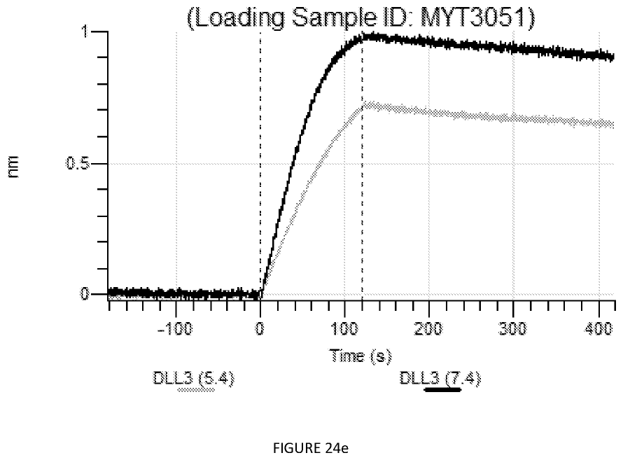

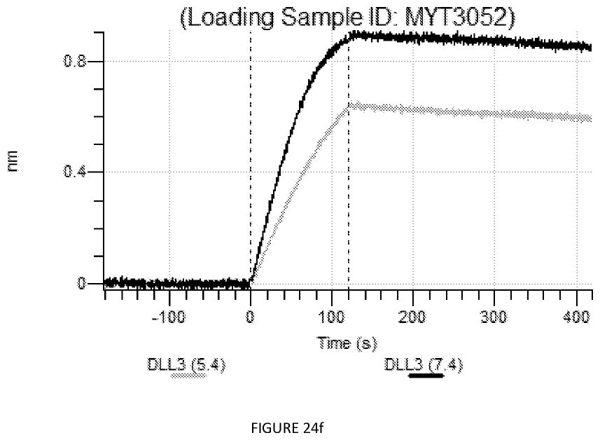

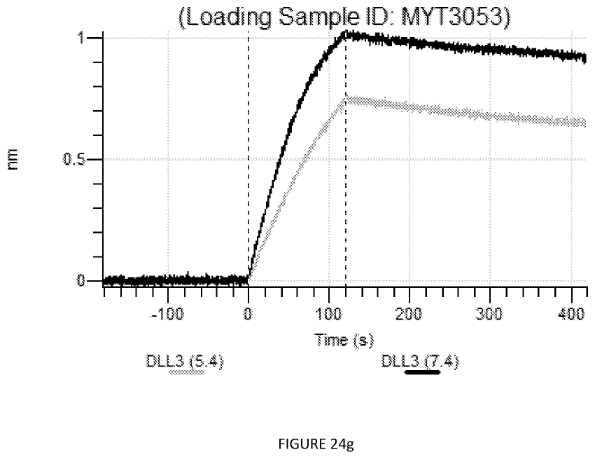

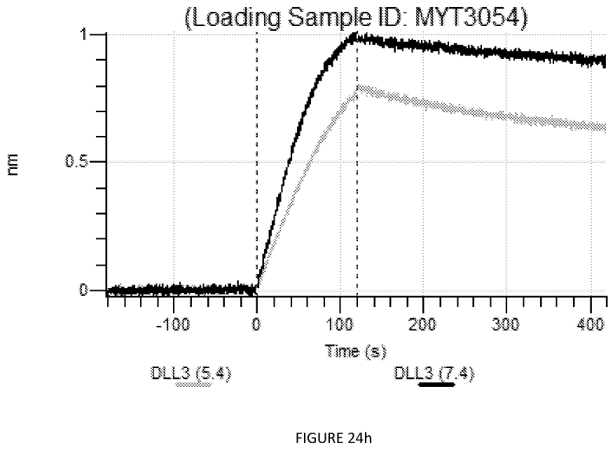

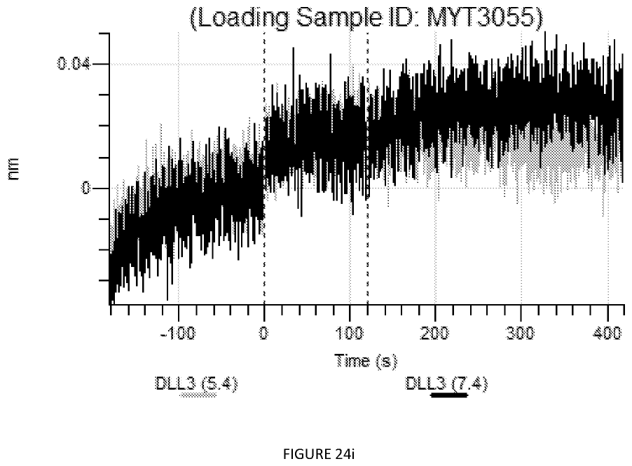

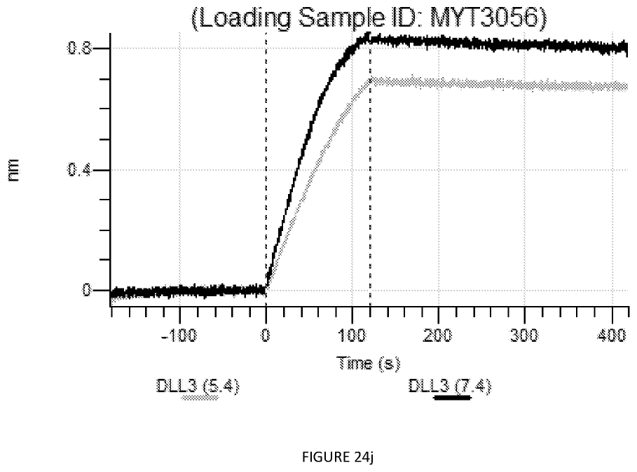

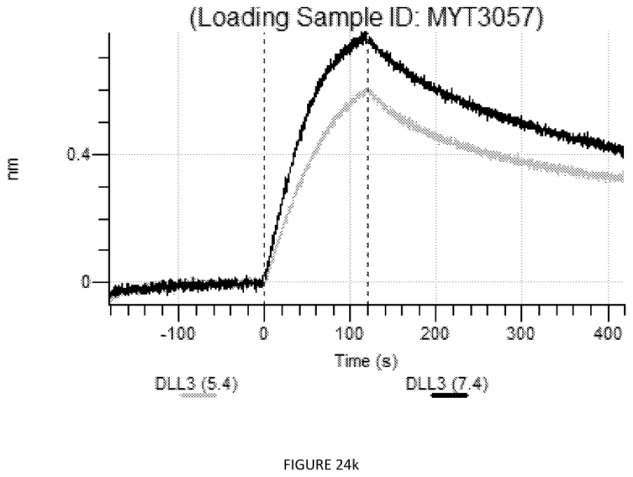

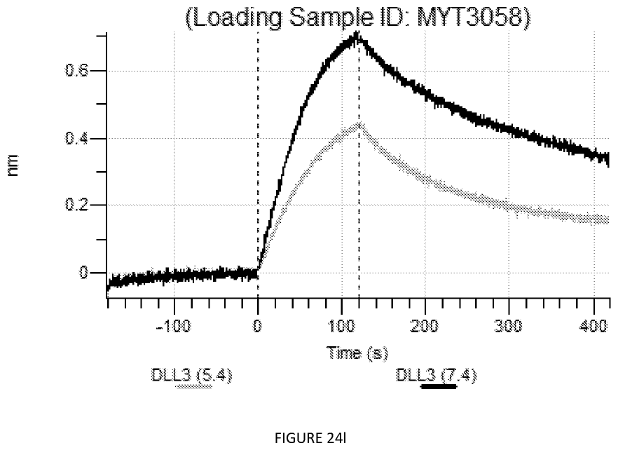

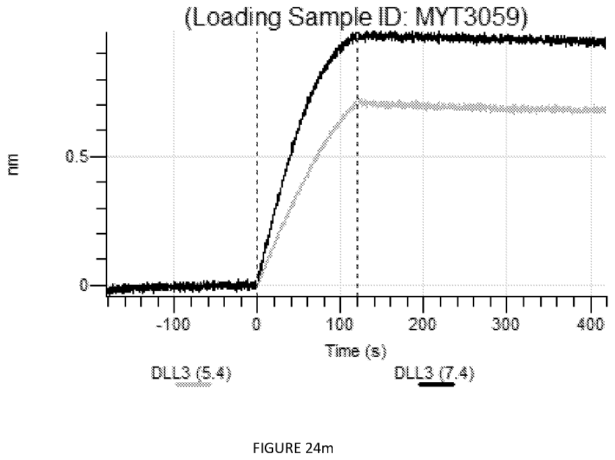

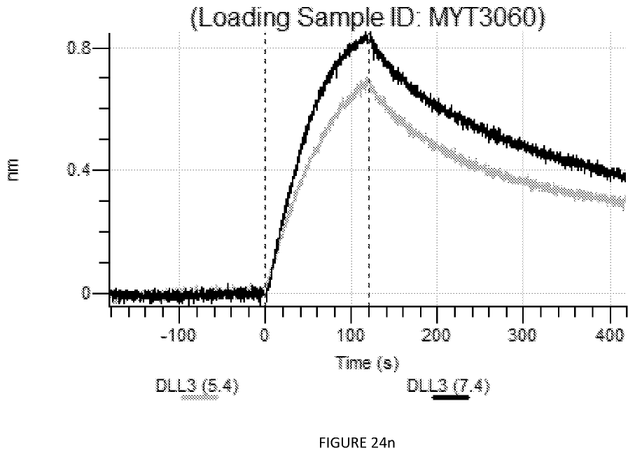

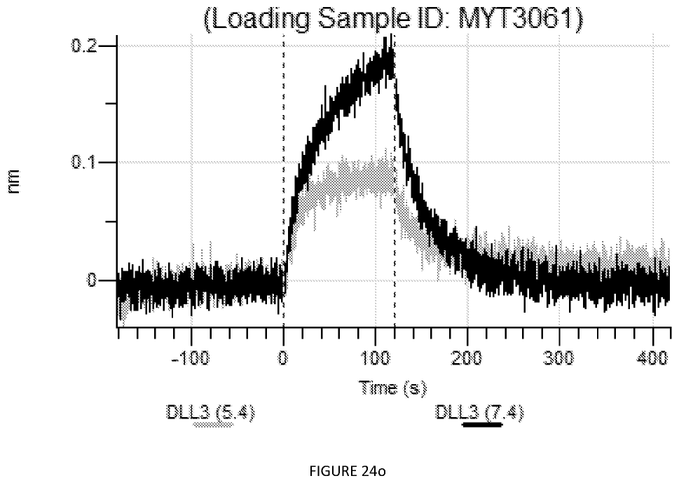

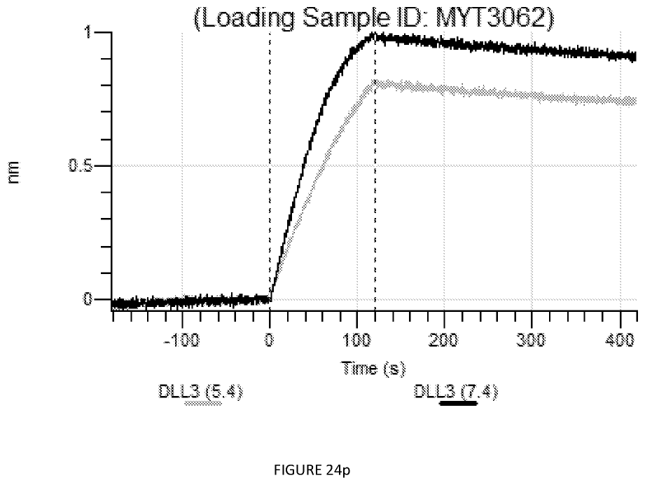

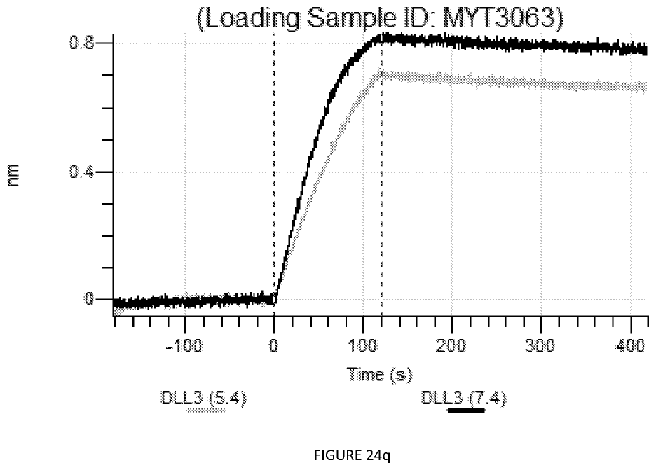

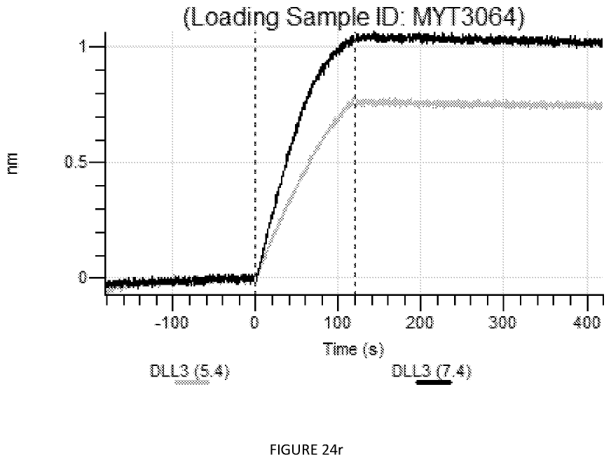

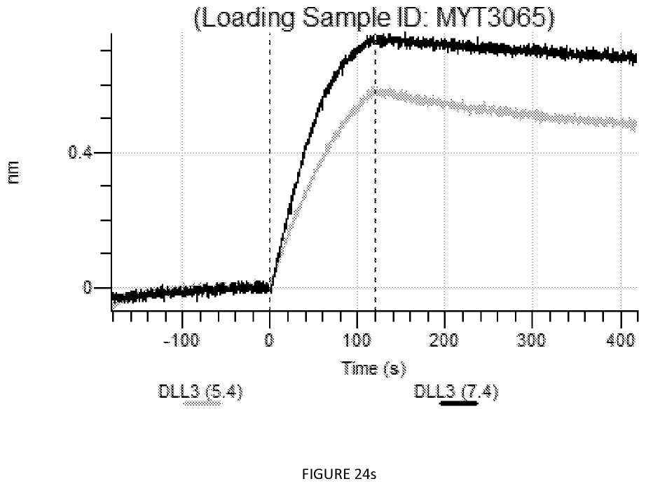

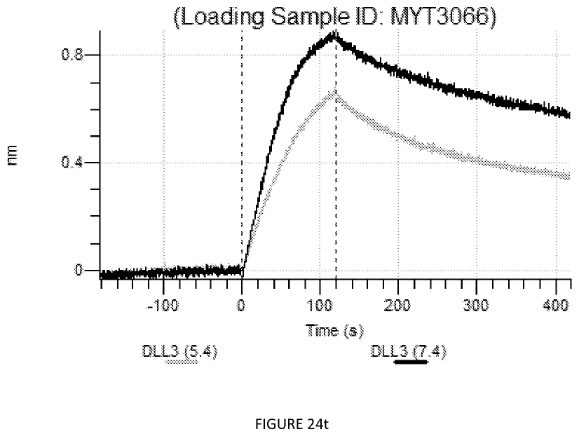

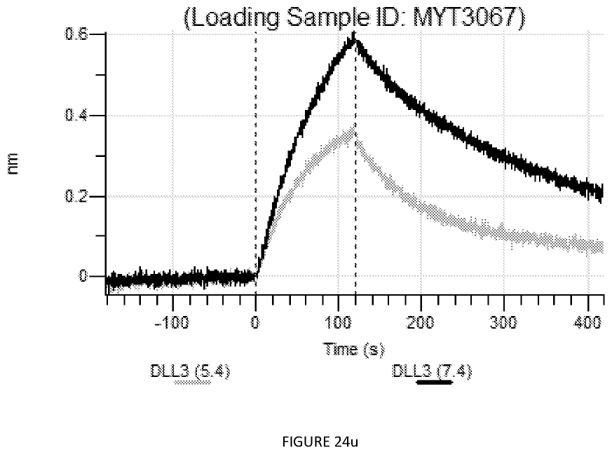

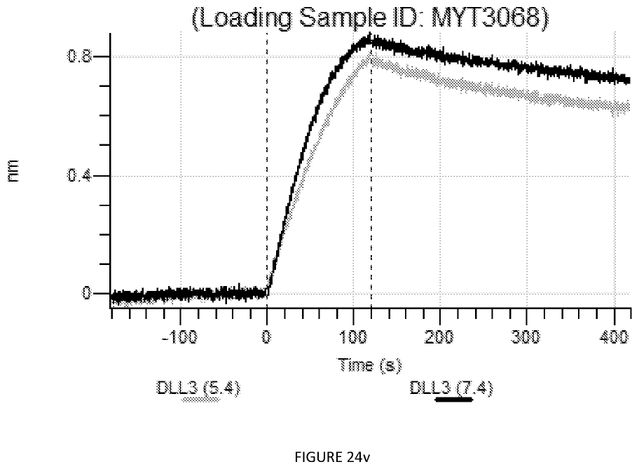

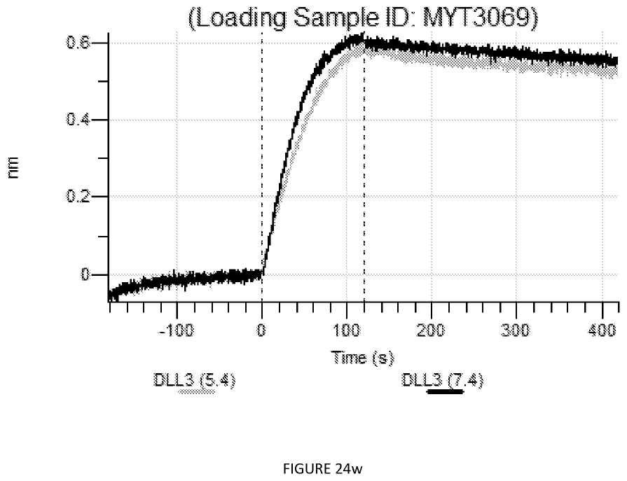

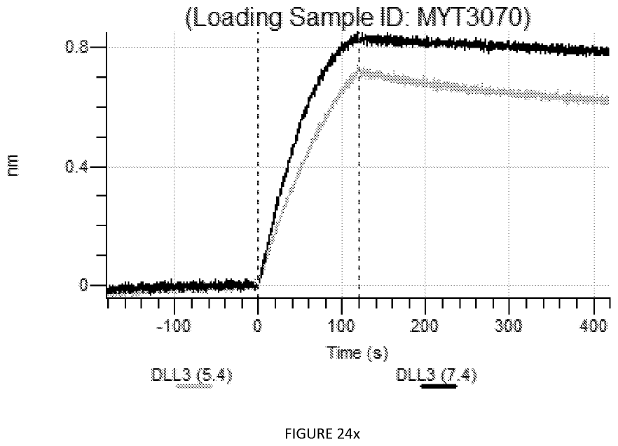

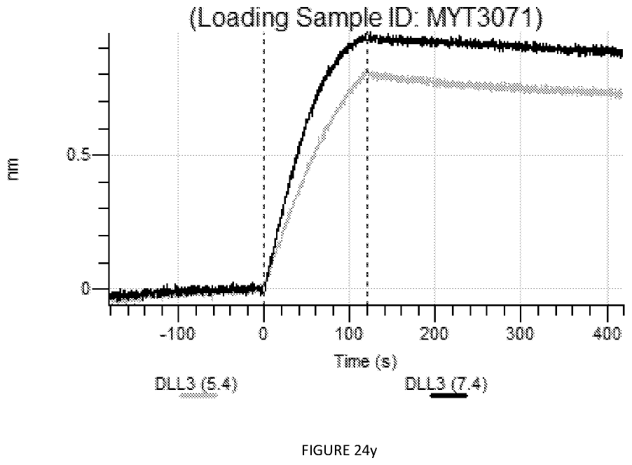

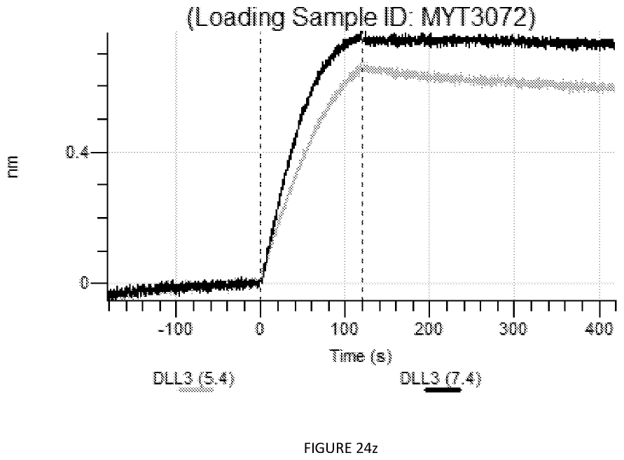

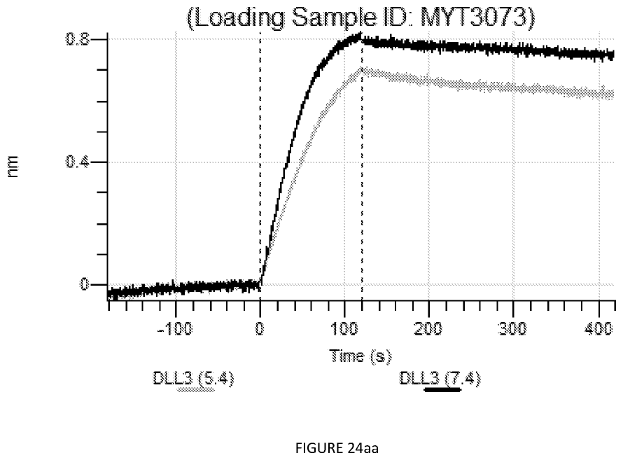

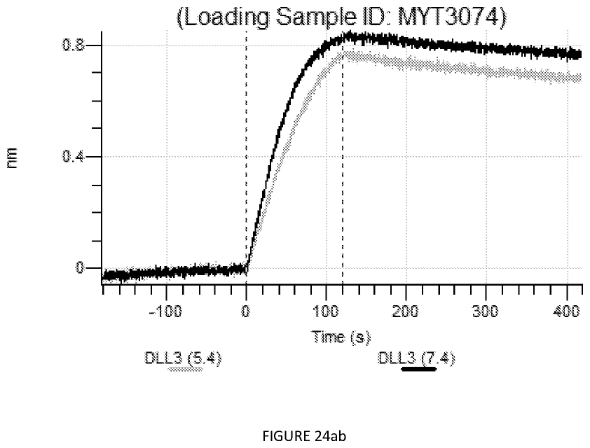

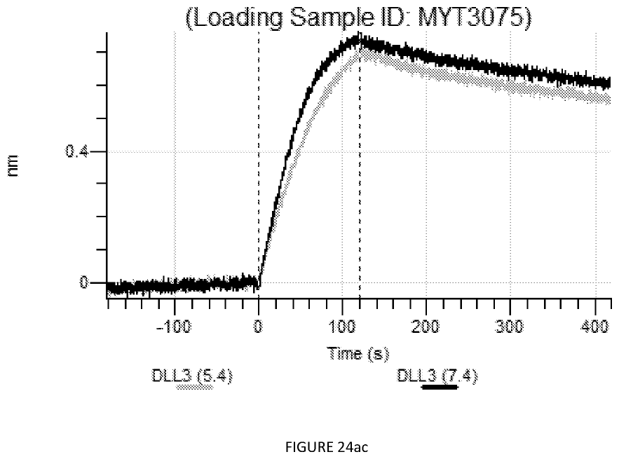

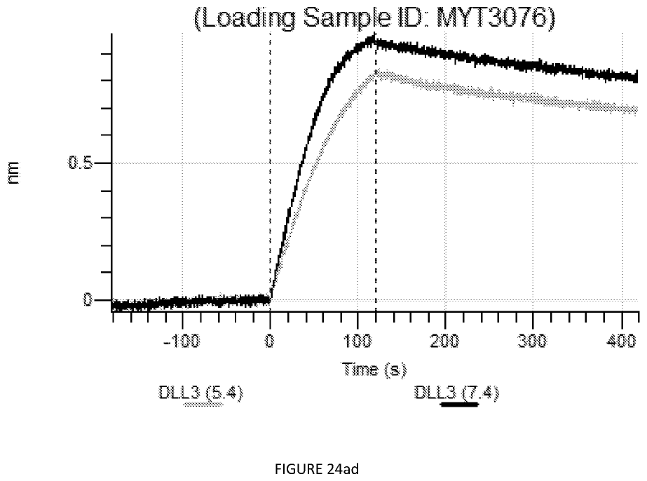

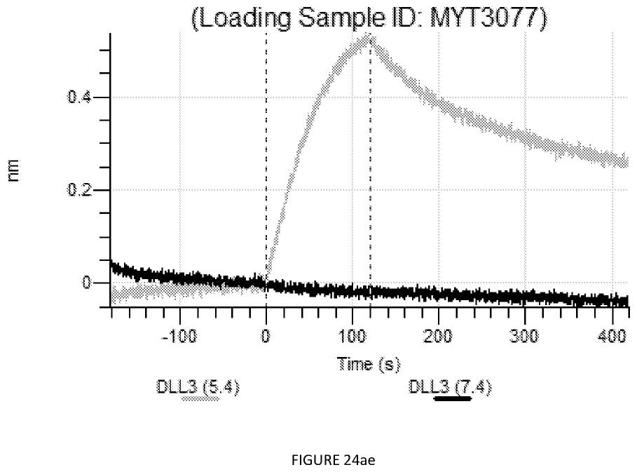

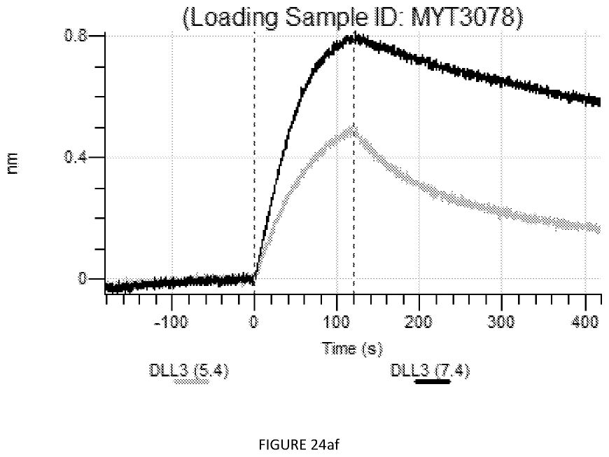

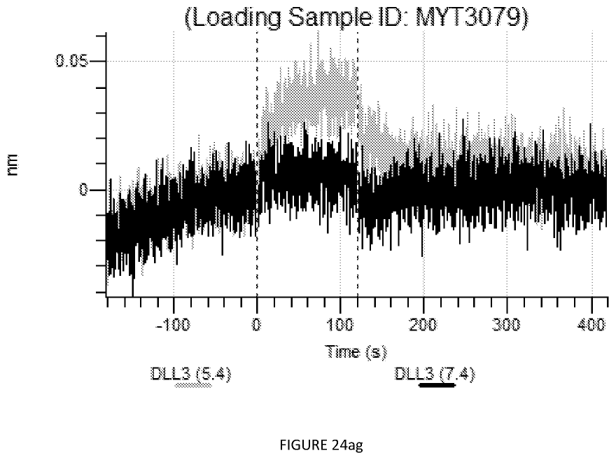

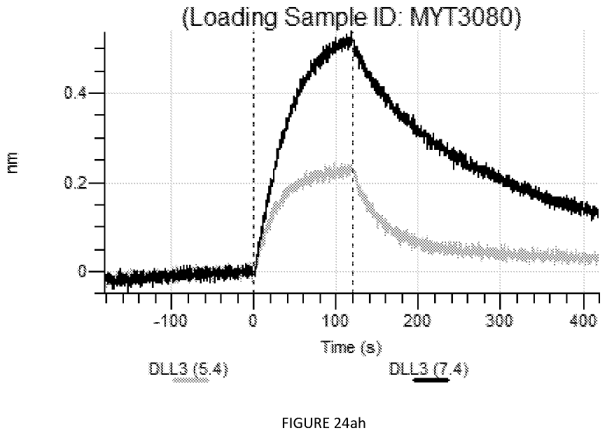

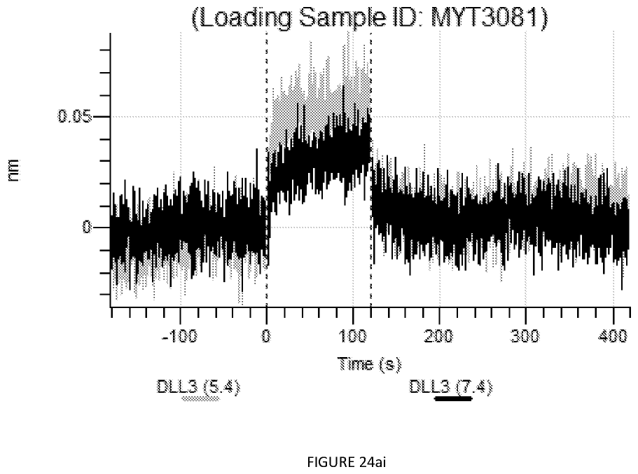

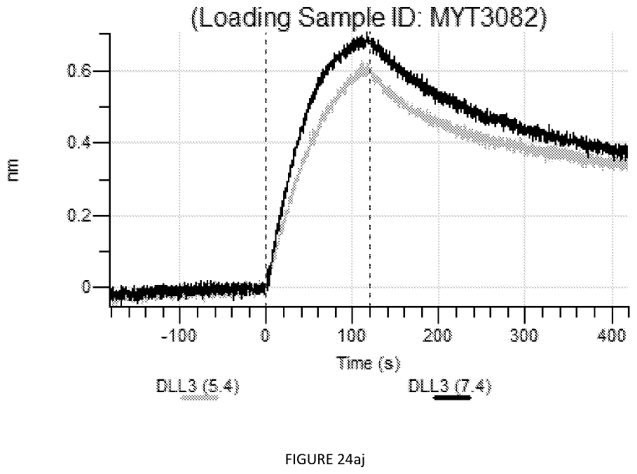

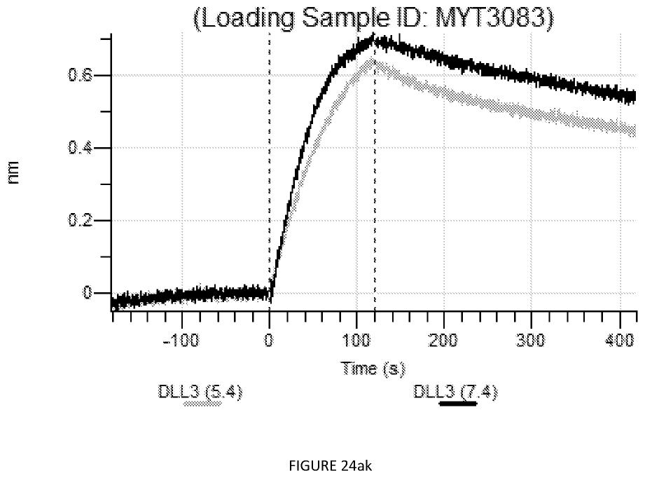

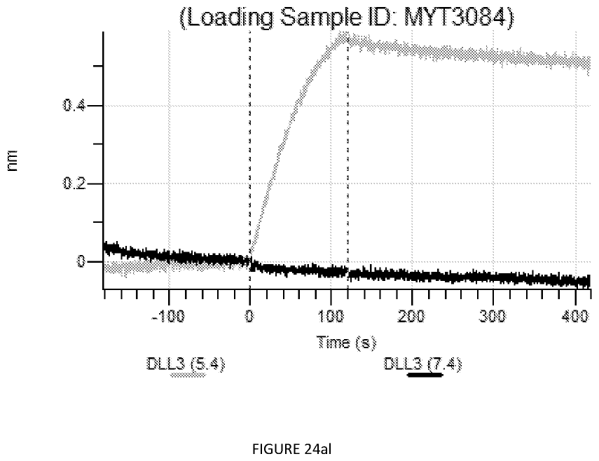

Multiple DLL3-binding monoclonal antibodies have been described in the literature and can be used as a template for engineering pH-dependent binding [Saunders L R (2016) “A DLL3-targeted antibody-drug conjugate eradicates high-grade pulmonary neuroendocrine tumor-initiating cells in vivo” Science Translational Medicine 7(302):302ra136]. SC16.4 (U.S. Pat. No. 9,089,617 B2) (Heavy chain SEQ ID NO: 119, Light chain SEQ ID NO: 120) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3048-MYT3084). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 23) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (i.e., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 24, were selected for further analysis (MYT3054, MYT3057, MYT3058, MYT3060, MYT3061, MYT3065, MYT3066, MYT3067, MYT3068, MYT3070, MYT3072, MYT3073, MYT3075, MYT3076, MYT3077, MYT3078, MYT3080, MYT3082, MYT3083, and MYT3084). It was also noted that while some histidine and alanine mutations obliterated DLL3 binding (e.g., MYT3055, MYT3079, and MYT3081), others were tolerated with little (e.g., less than 1-fold change in dissociation constant KD or dissociation rate) or no change in DLL3 binding kinetics (e.g., MYT3048, MYT3049, MYT3050, MYT3051, MYT3052, MYT3053, MYT3056, MYT3059, MYT3062, MYT3063, MYT3064, MYT3069, MYT3071, and MYT3074) Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 15. Construction and Screening of pH-Engineered DLL3 ABPCs

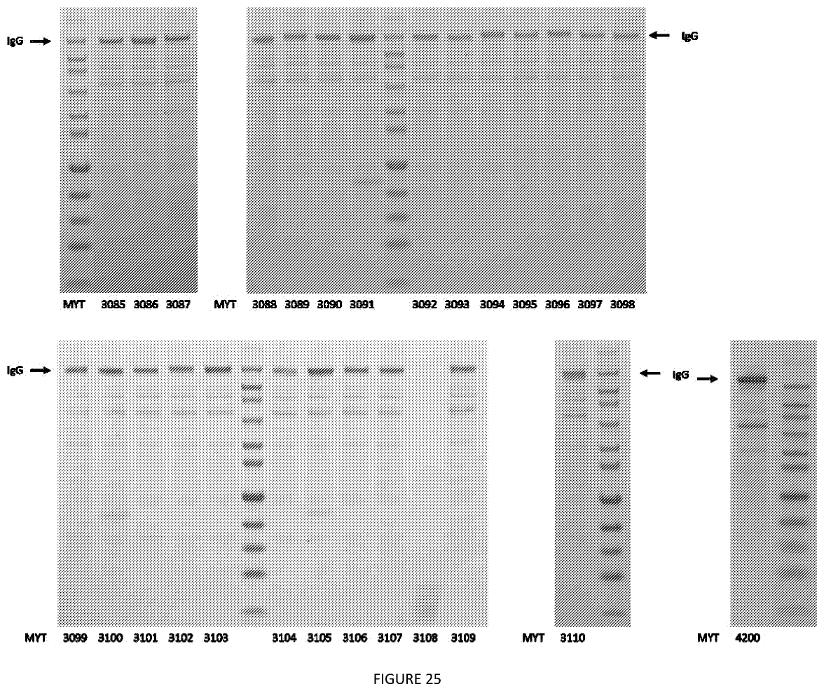

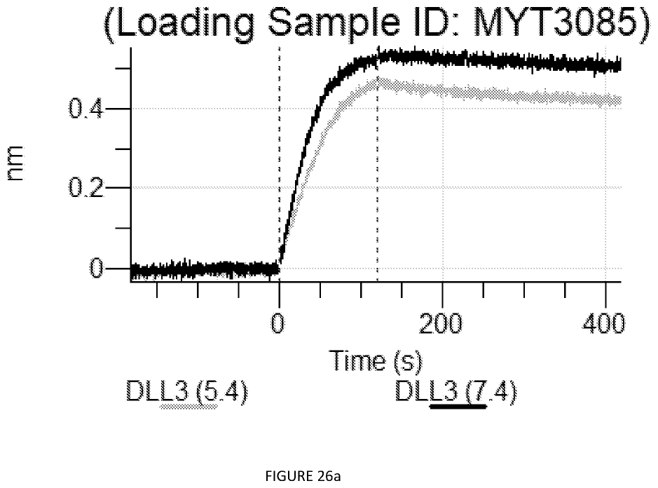

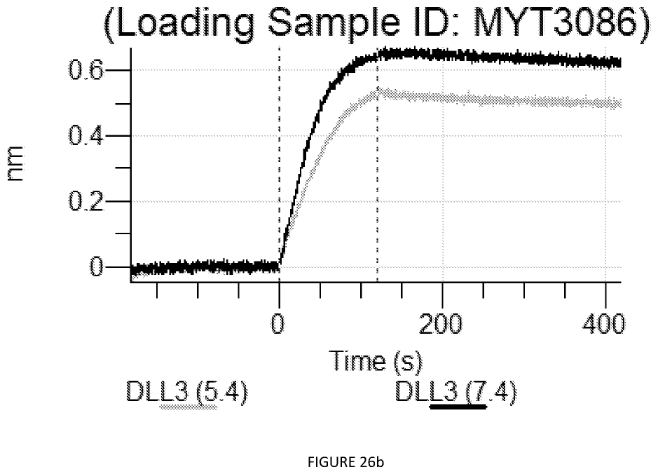

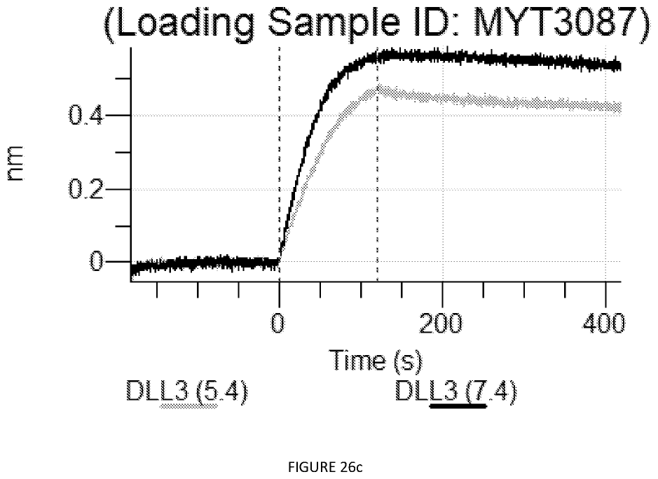

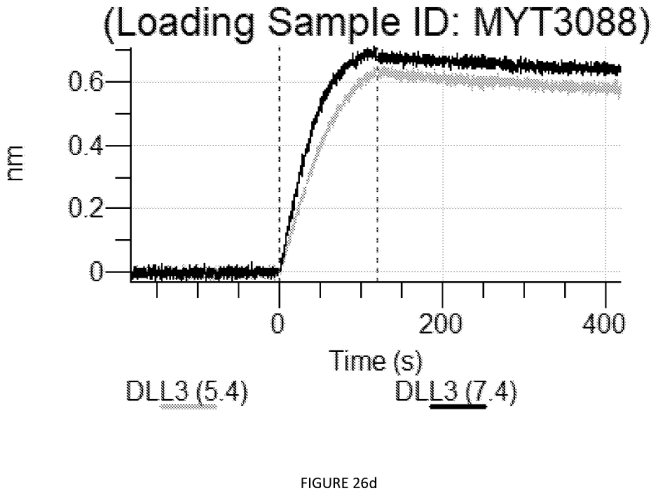

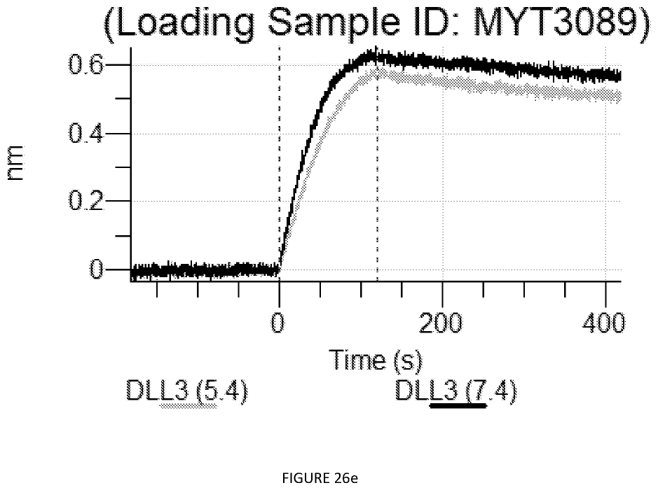

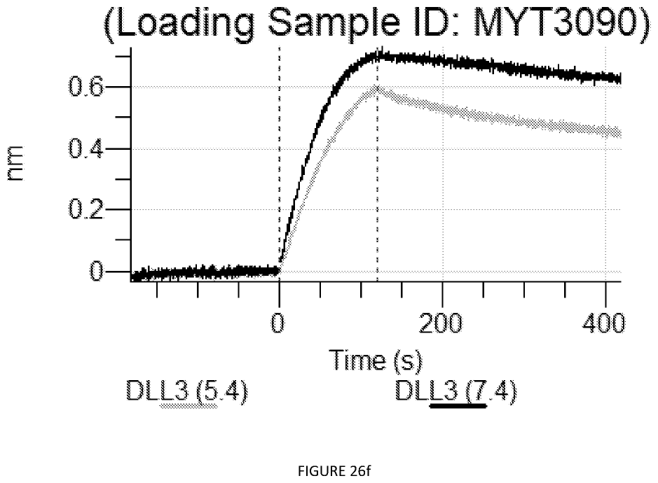

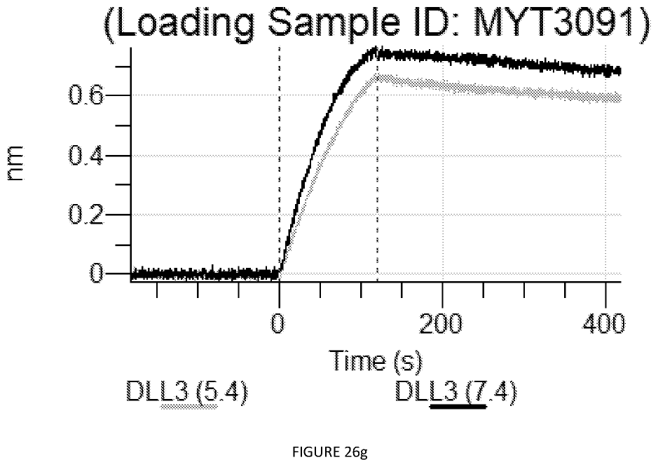

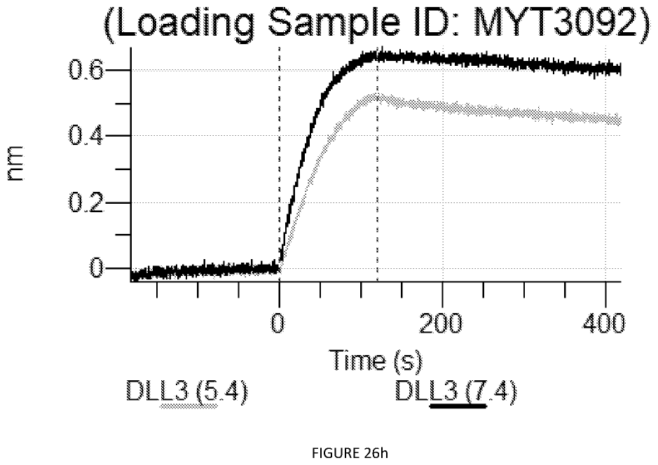

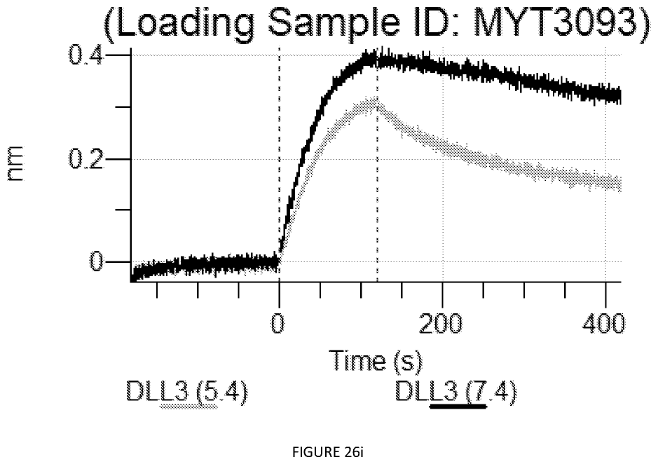

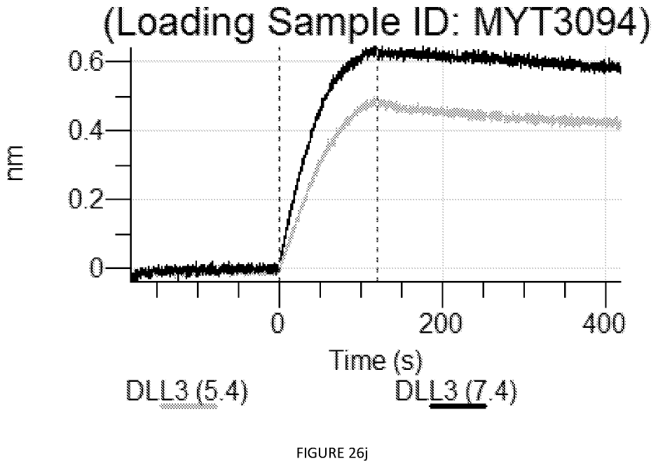

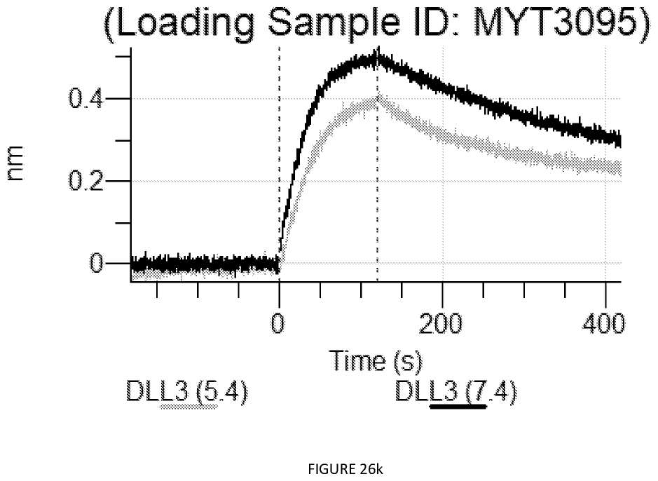

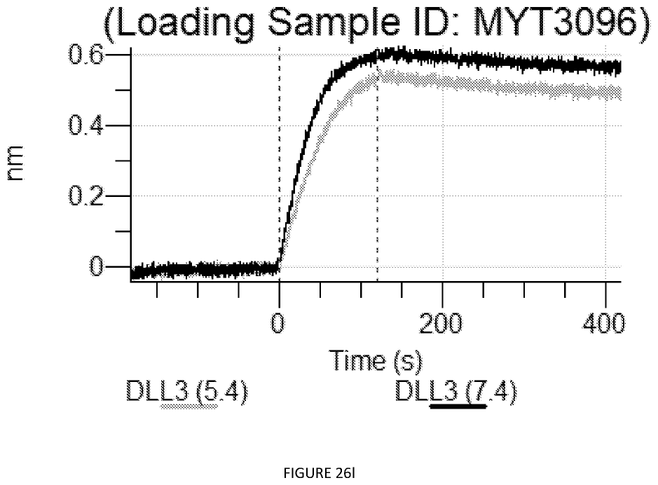

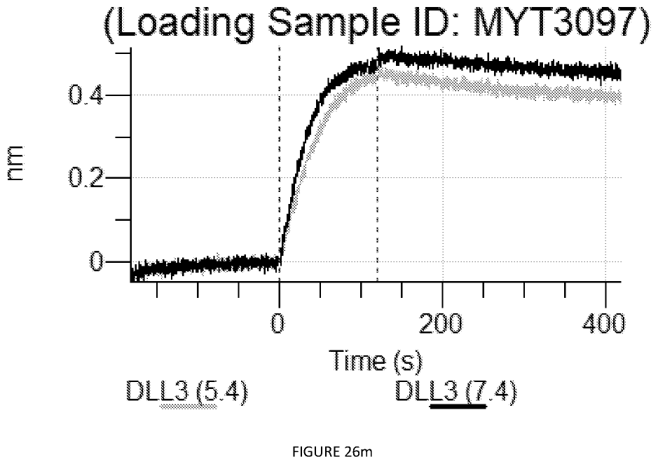

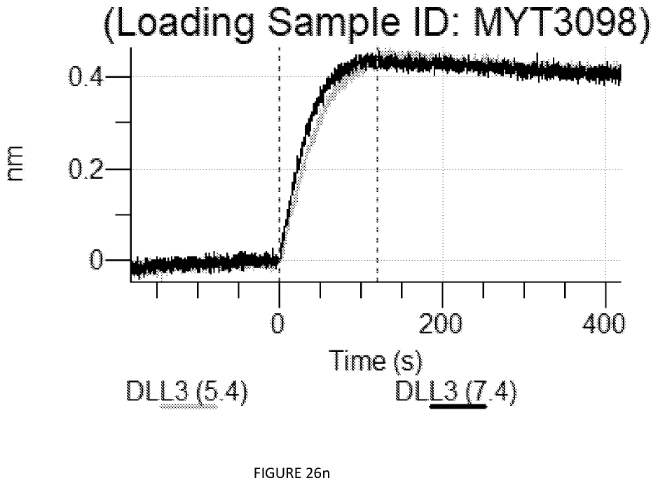

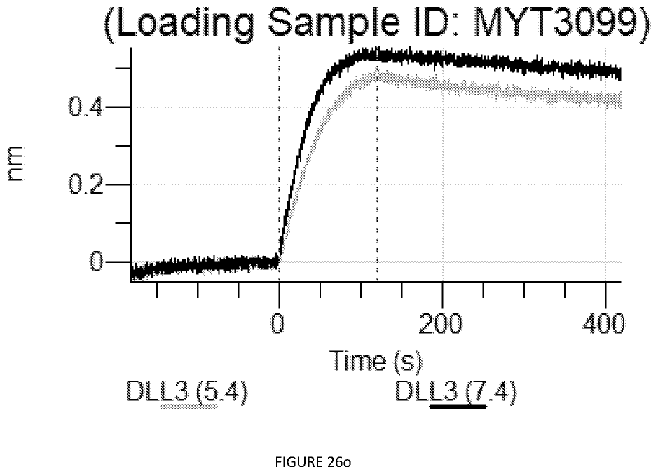

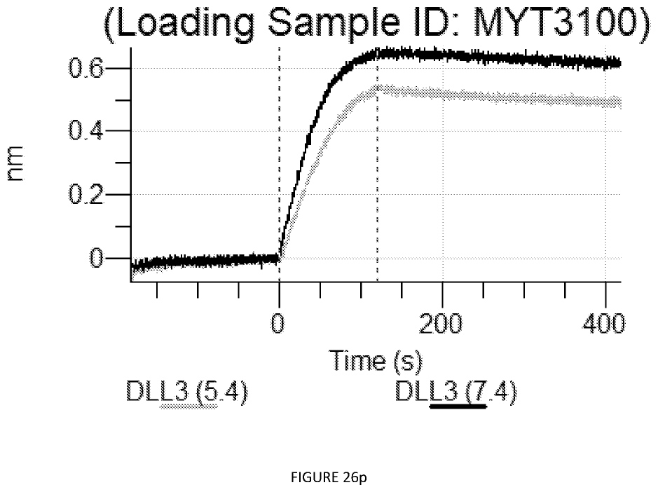

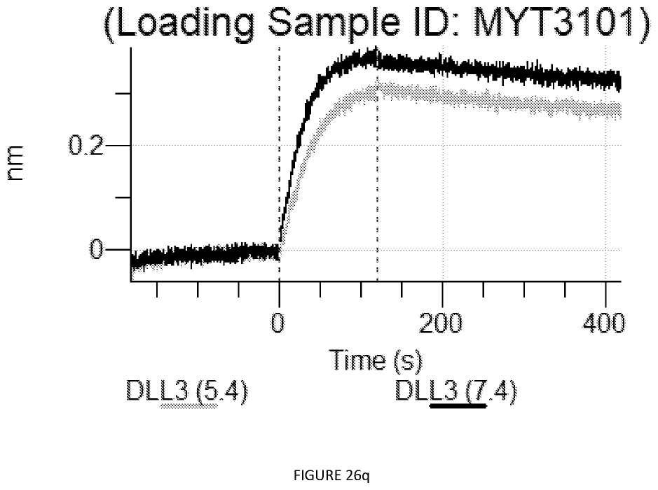

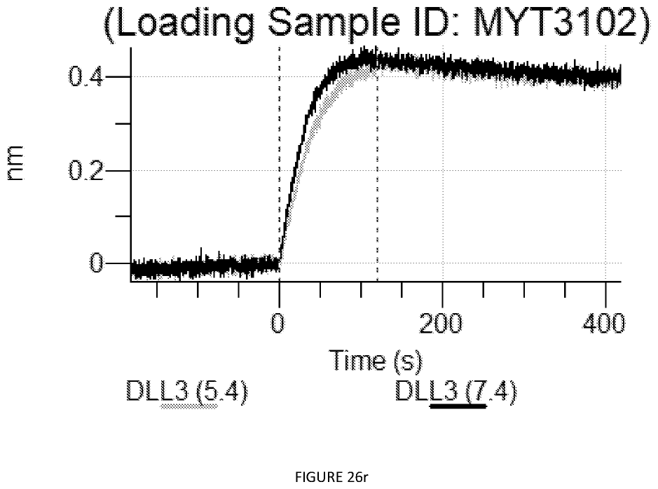

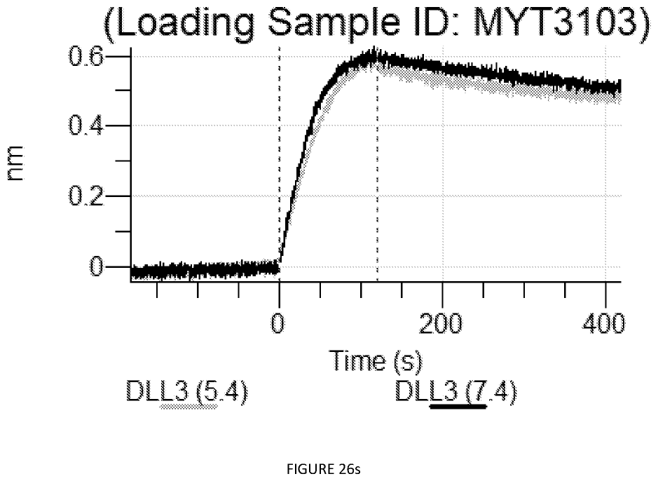

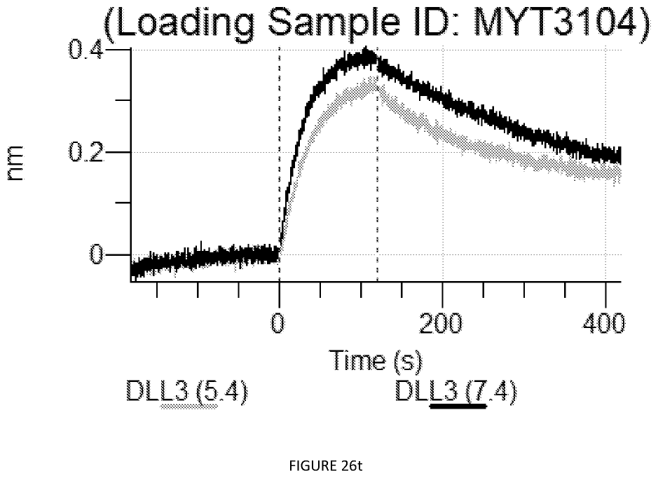

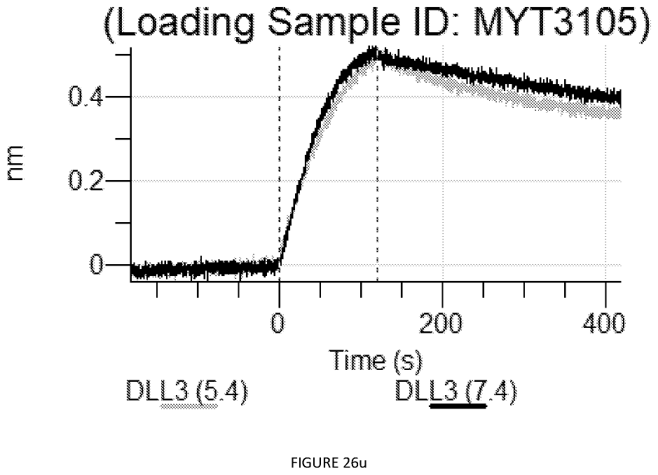

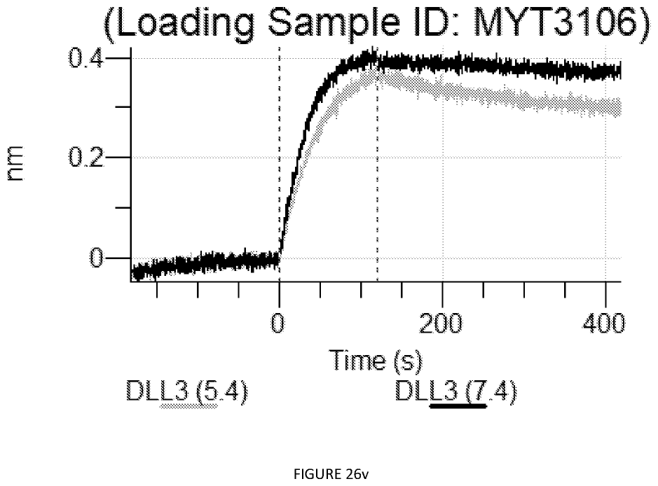

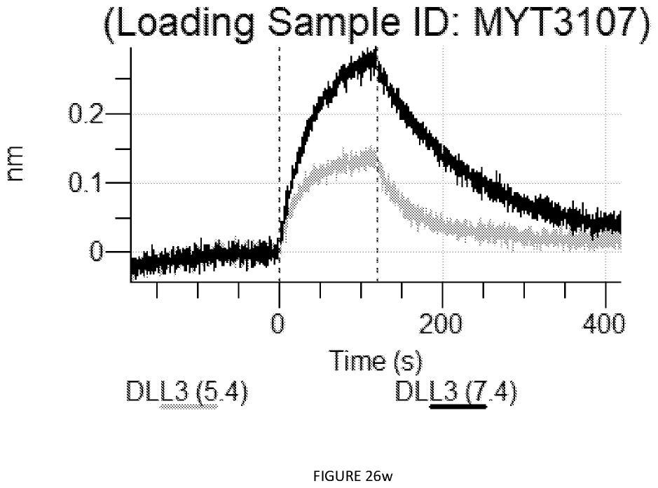

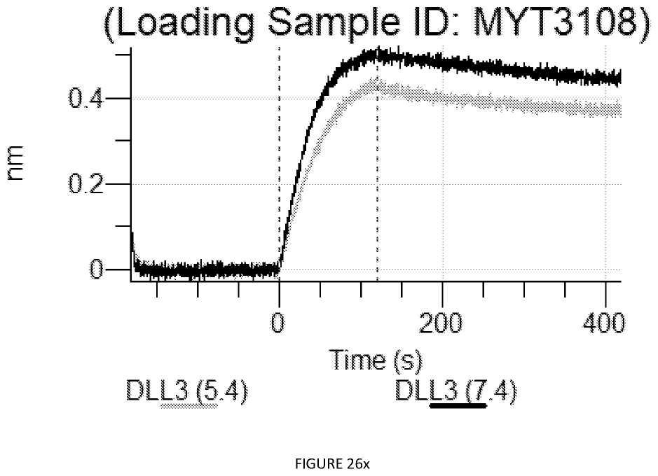

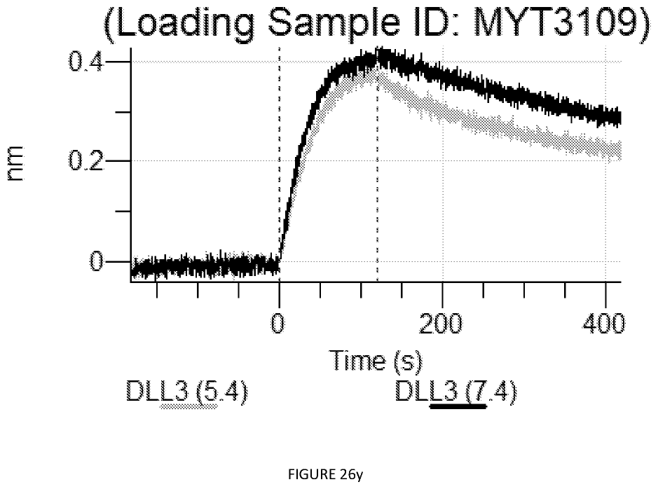

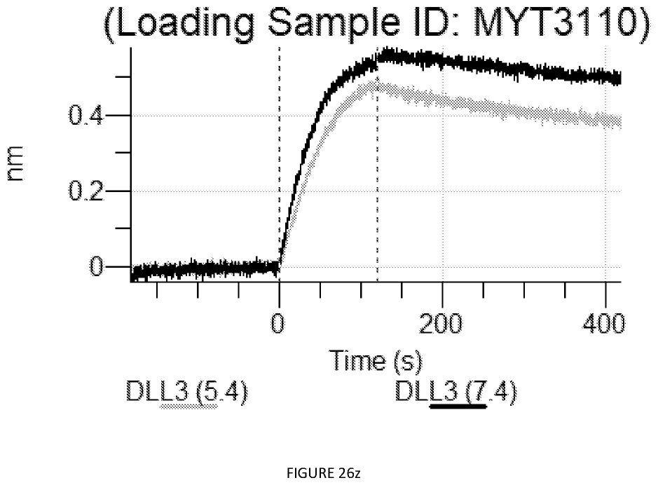

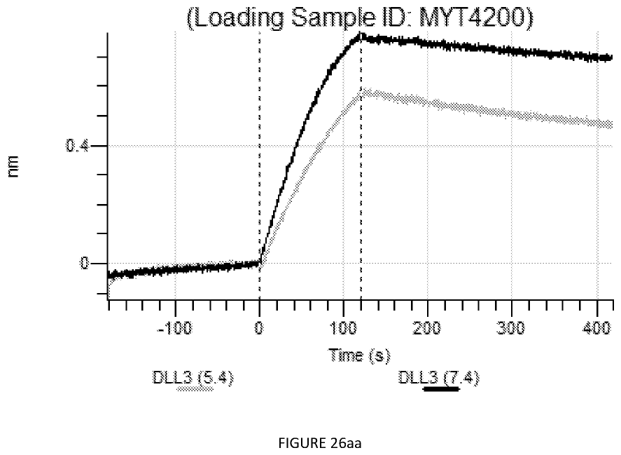

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.4 (U.S. Pat. No. 9,089,617 B2) (Heavy chain SEQ ID NO: 119, Light chain SEQ ID NO: 120) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3085-MYT3110 and MYT4200). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 25), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 26, were selected for further analysis (e.g., MYT3090, MYT3093, MYT3095, MYT3104, MYT3105, MYT3106, MYT3107, and MYT3109). It was also noted that while no histidine or alanine mutations obliterated DLL3 binding, others were tolerated with little (e.g., less than 1-fold change in dissociation constant KD or dissociation rate) or no change in DLL3 binding kinetics (e.g., MYT3085, MYT3086, MYT3087, MYT3088, MYT3089, MYT3091, MYT3092, MYT3094, MYT3096, MYT3097, MYT3098, MYT3099, MYT3100, MYT3101, MYT3102, MYT3103, MYT3108, and MYT3110). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 16. Construction and Screening of pH-Engineered DLL3 ABPCs

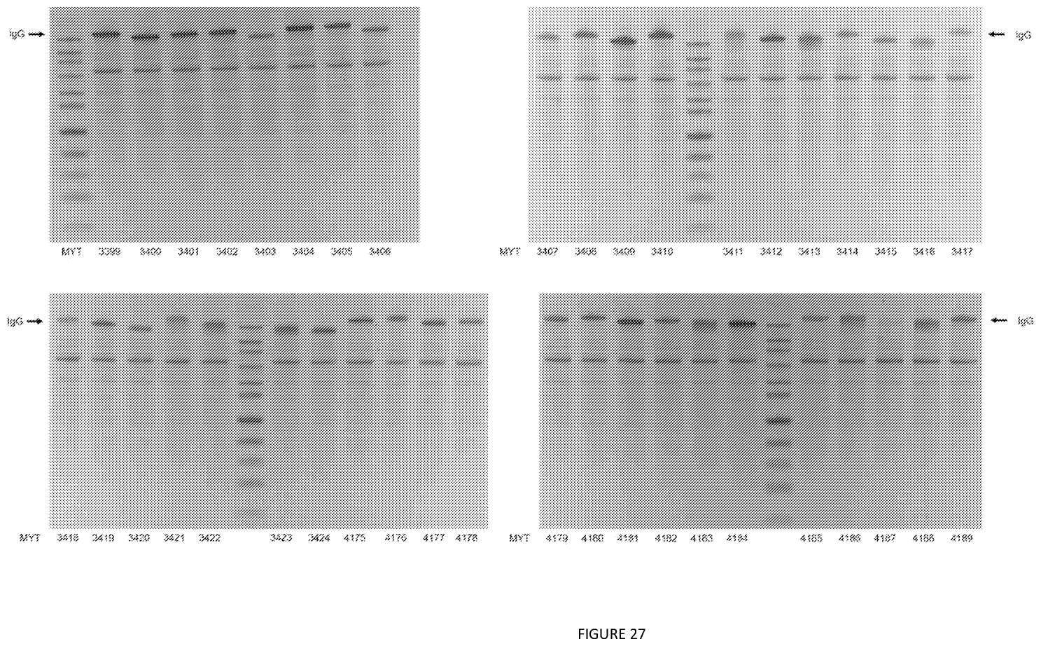

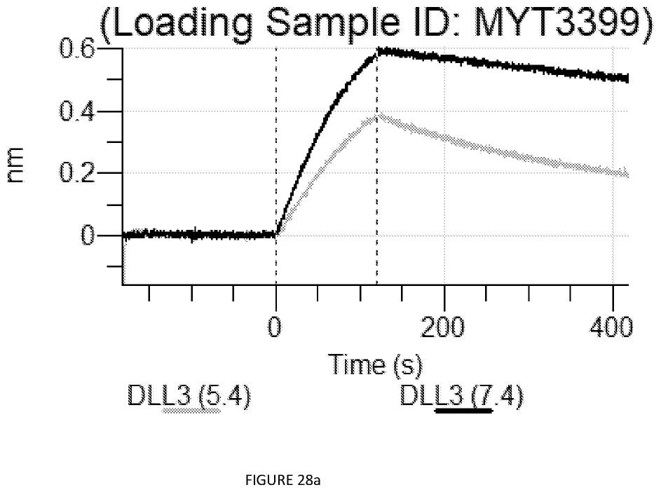

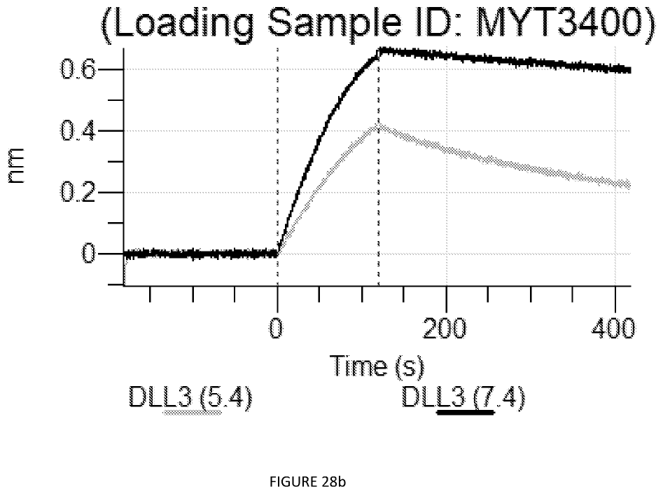

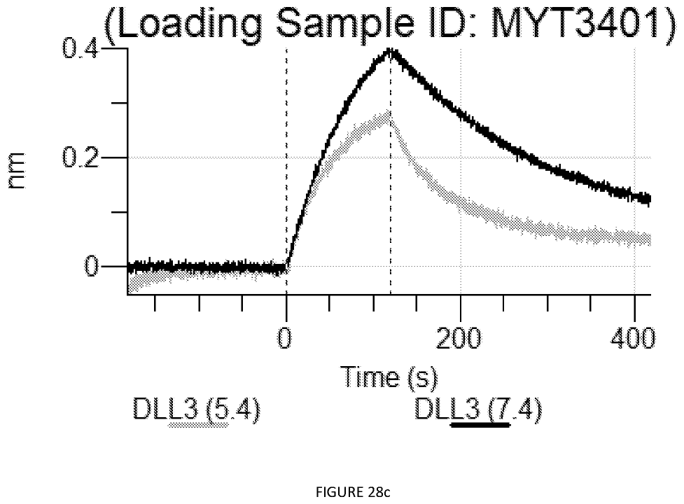

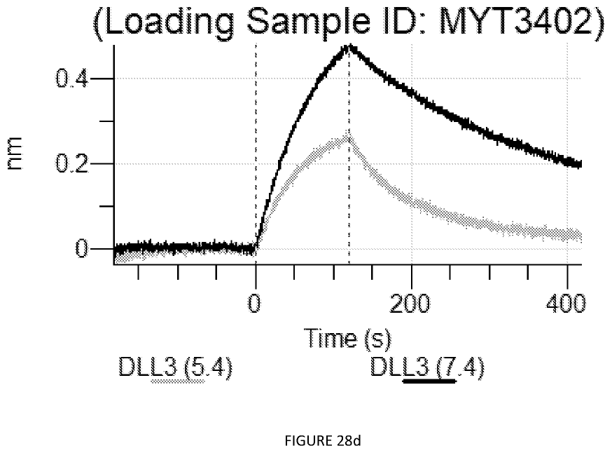

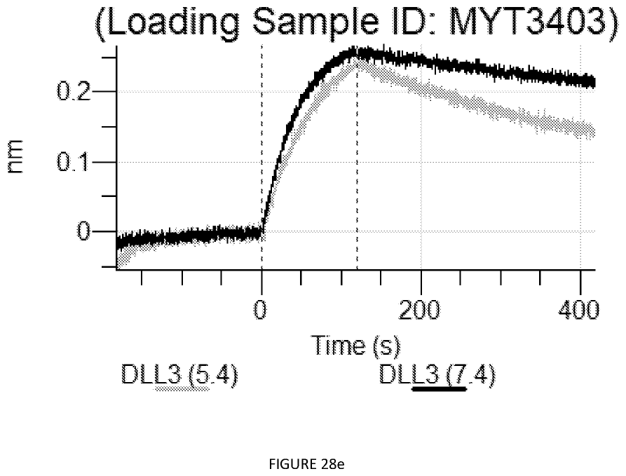

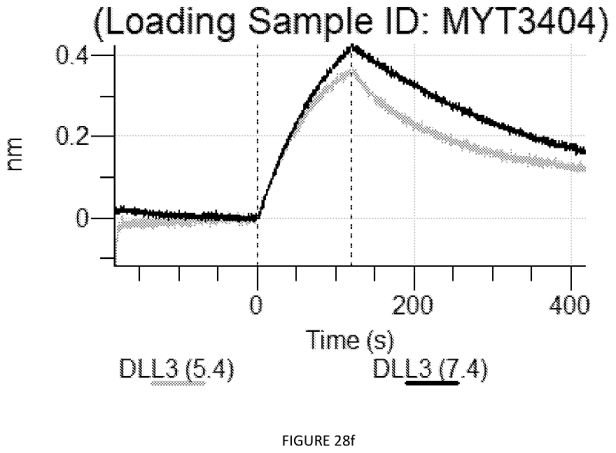

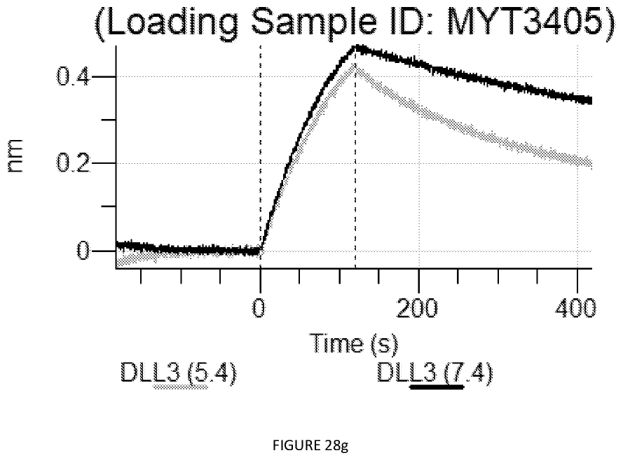

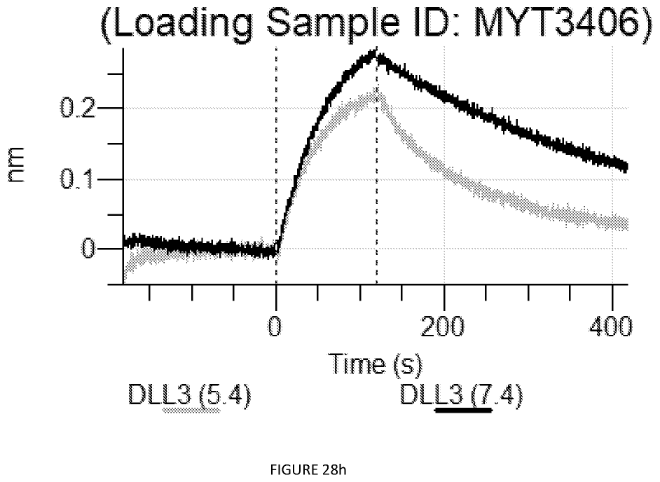

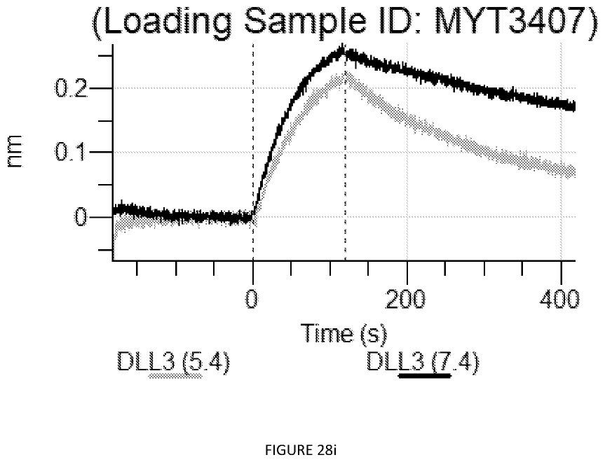

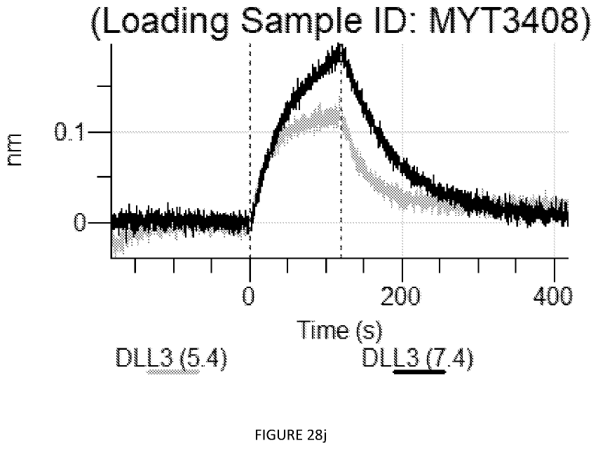

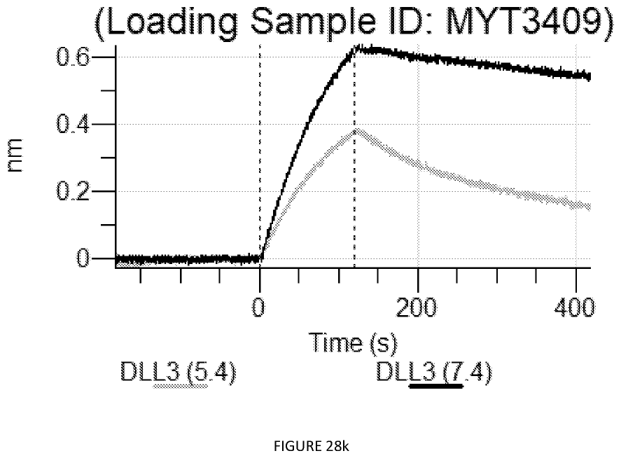

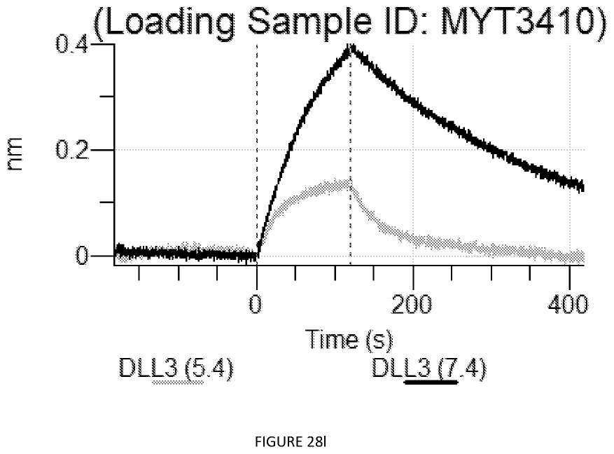

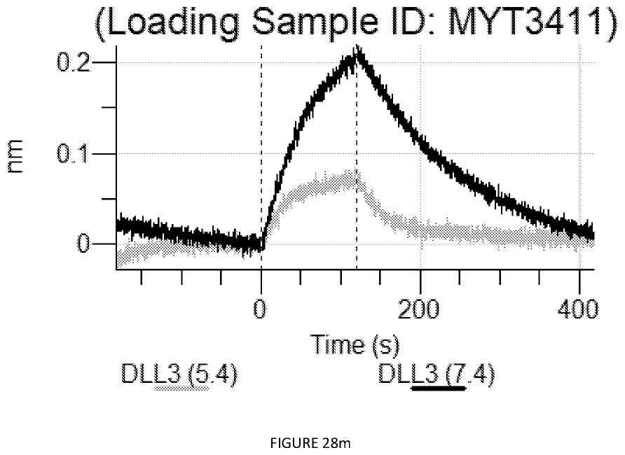

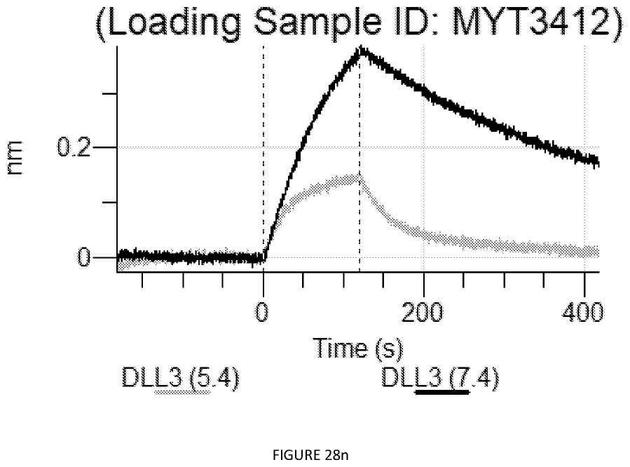

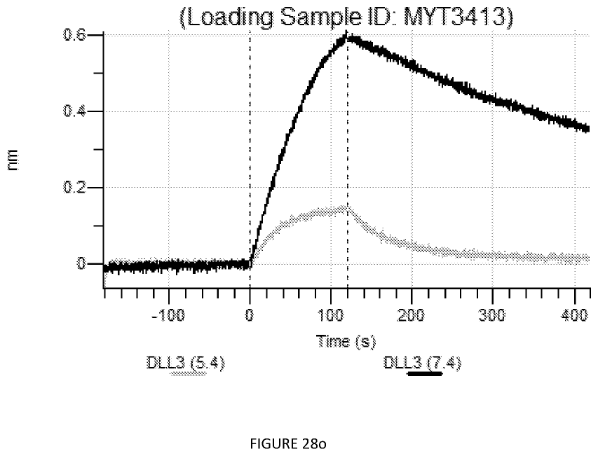

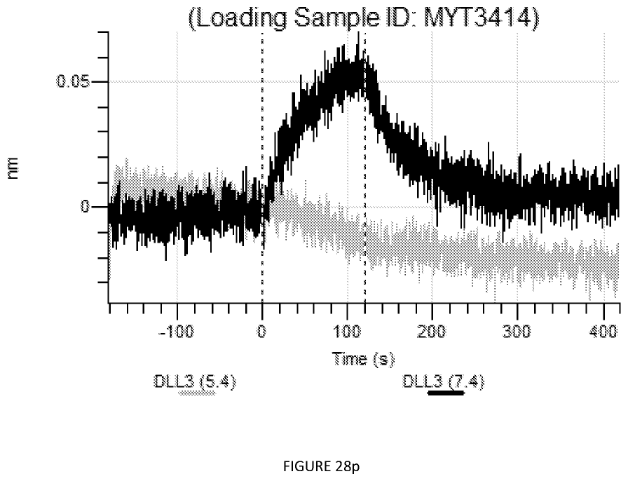

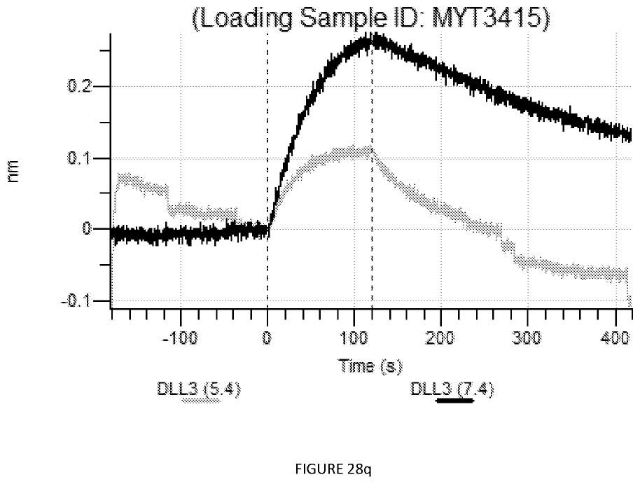

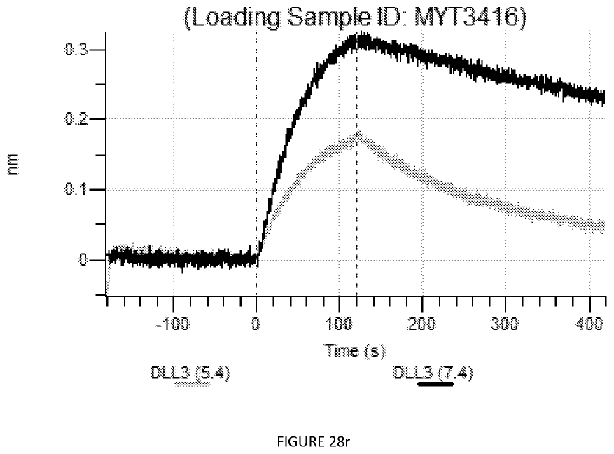

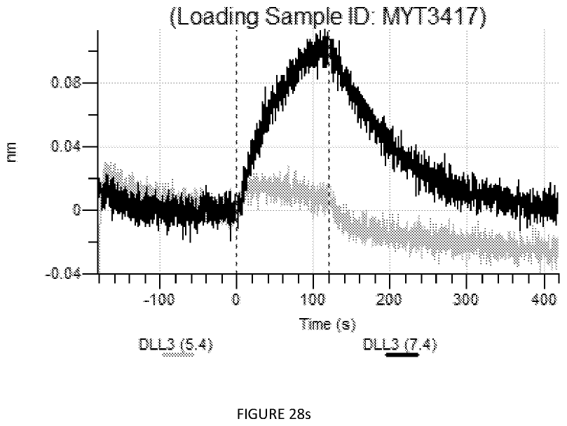

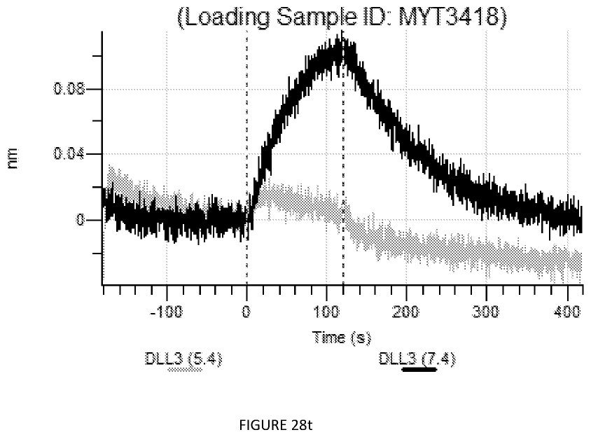

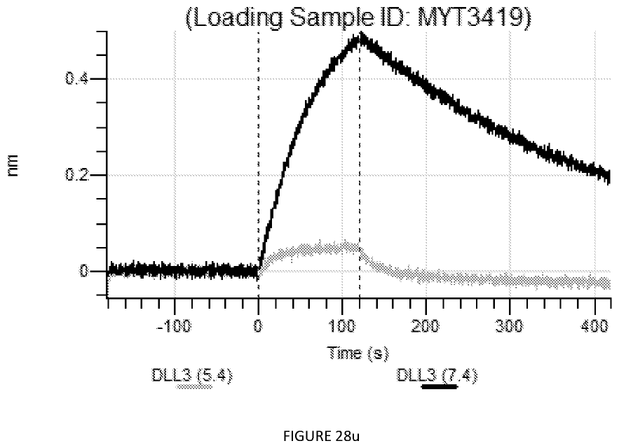

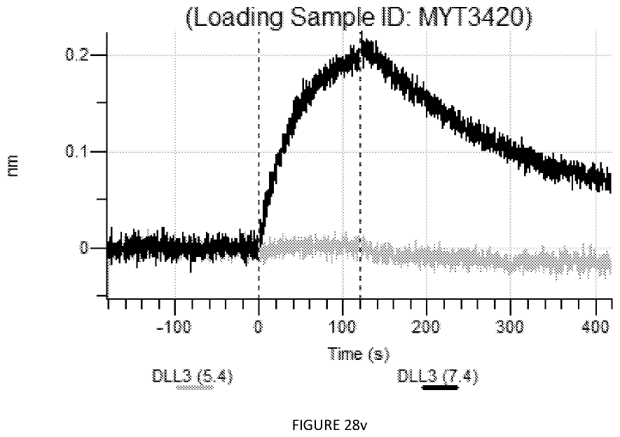

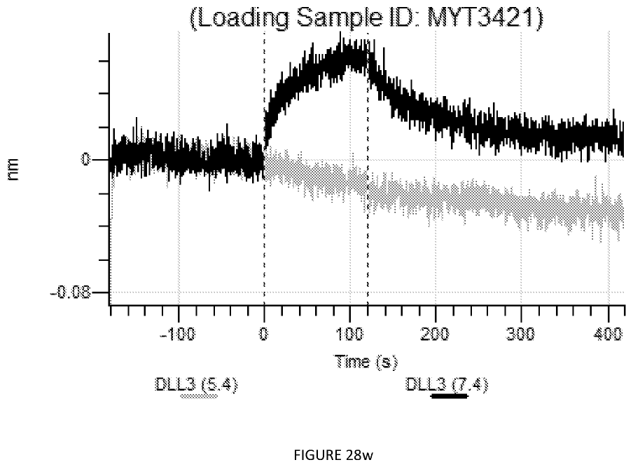

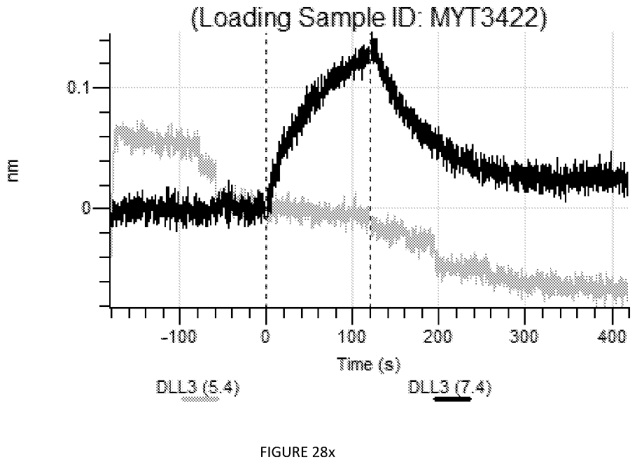

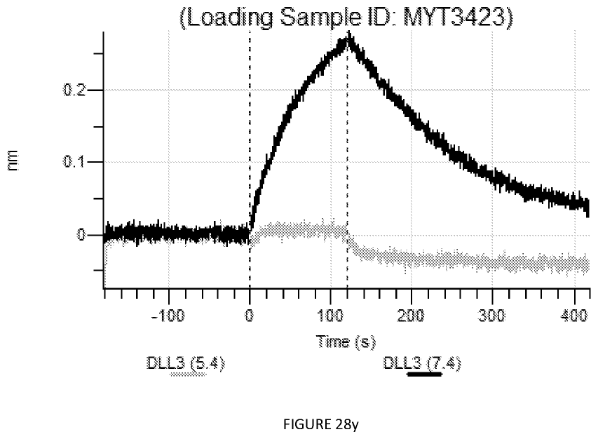

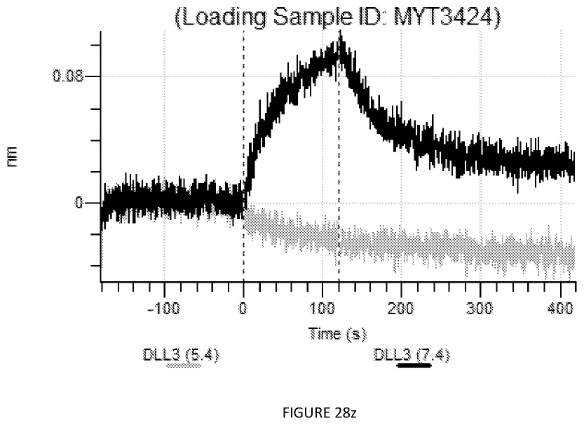

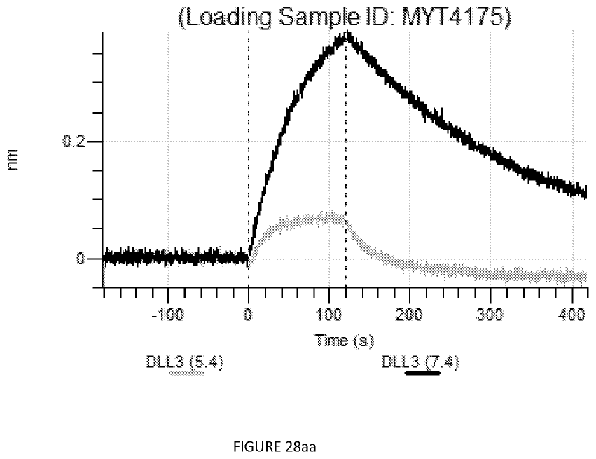

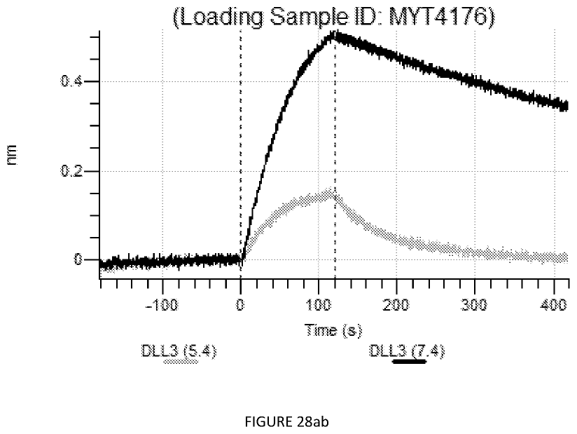

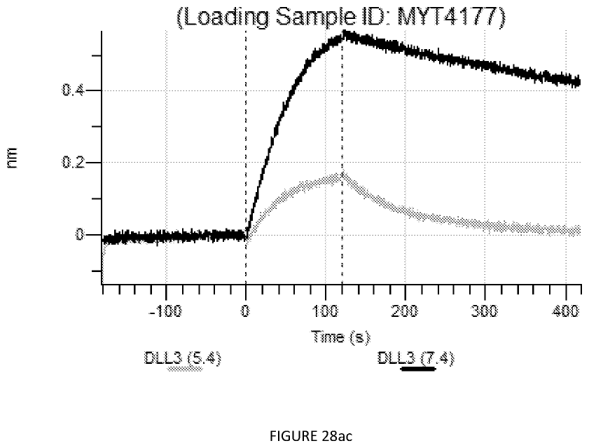

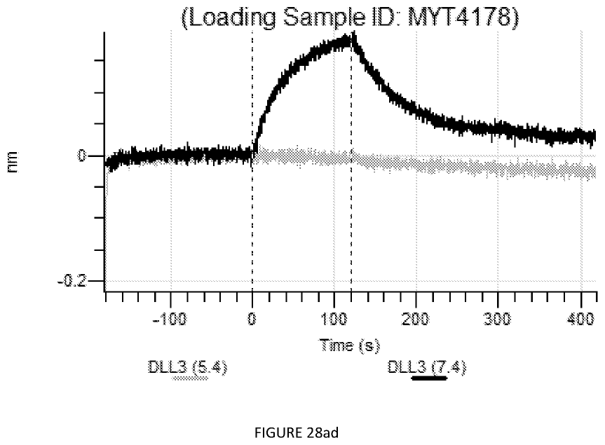

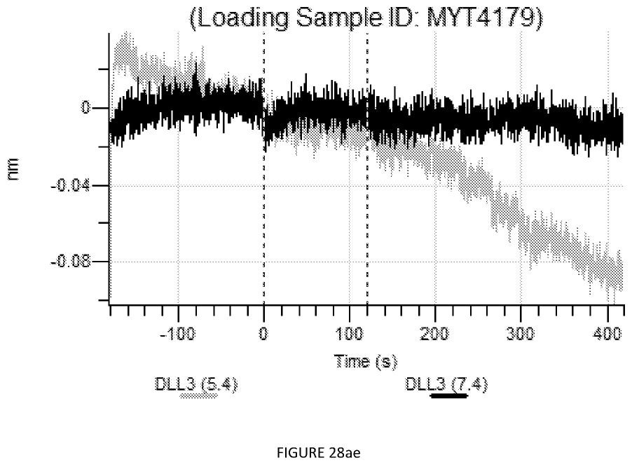

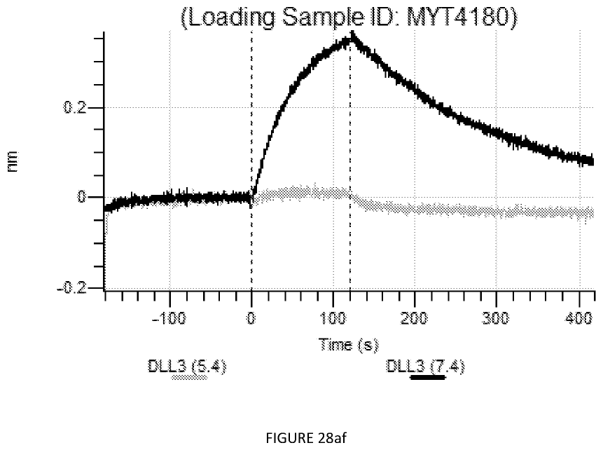

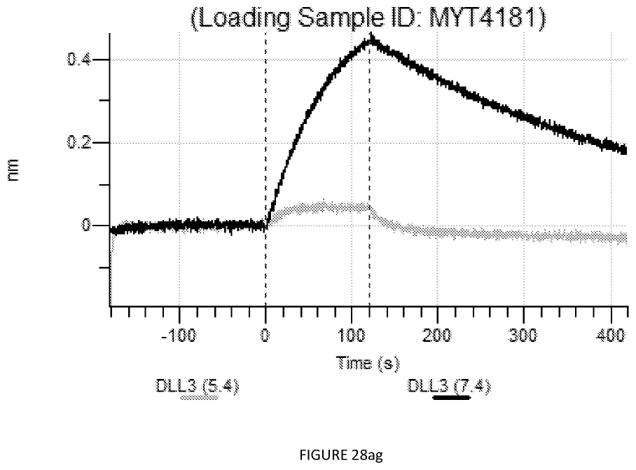

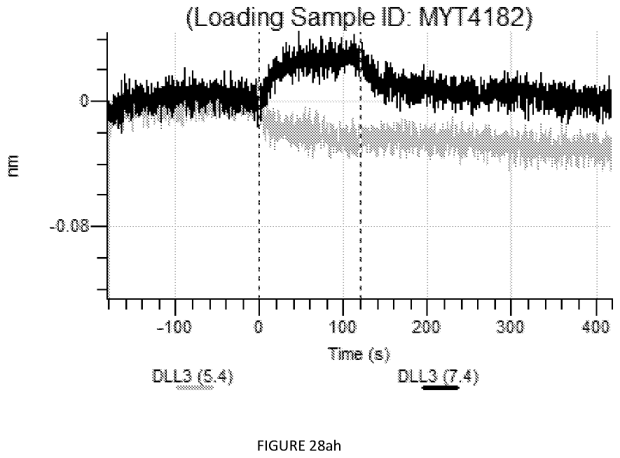

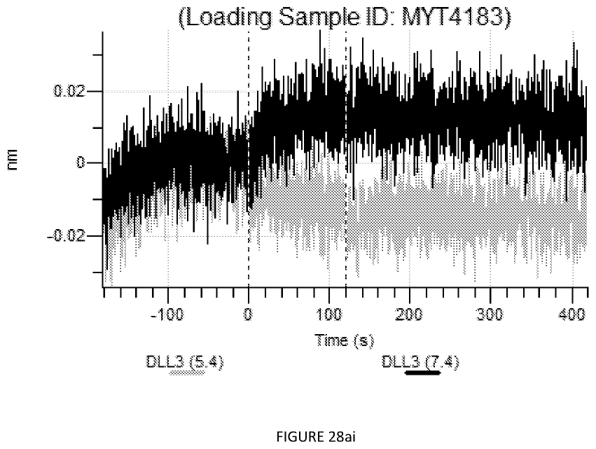

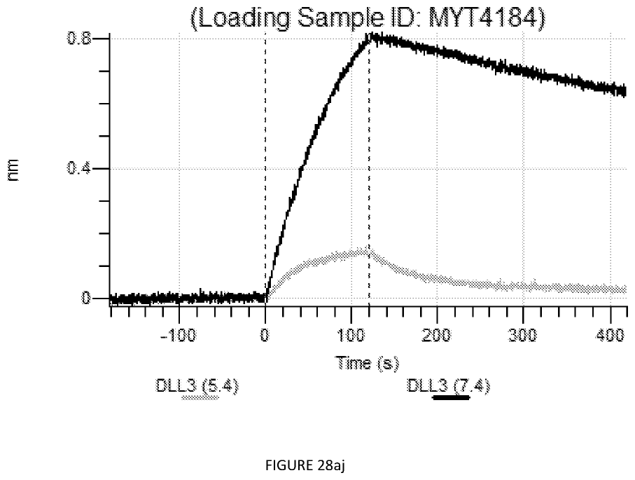

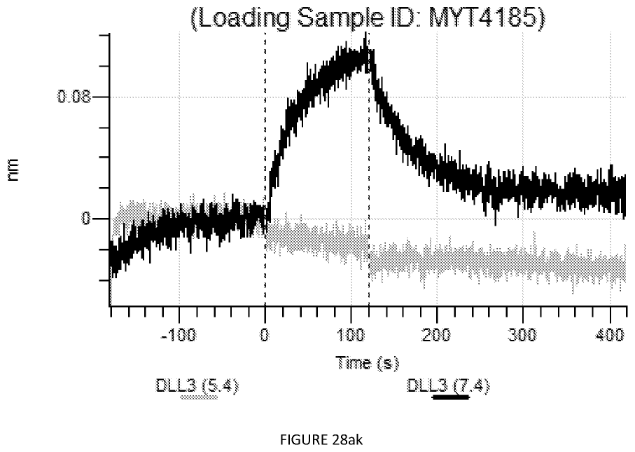

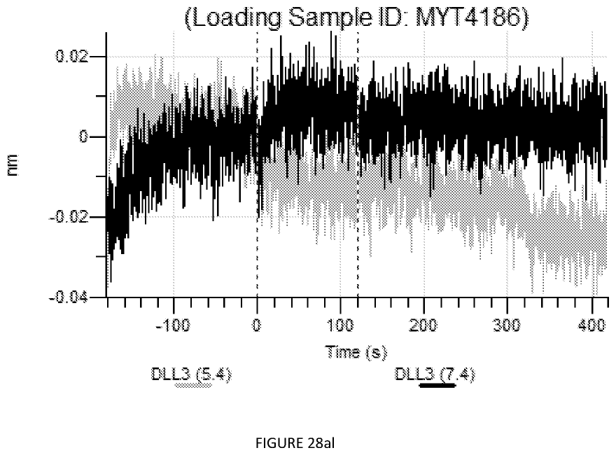

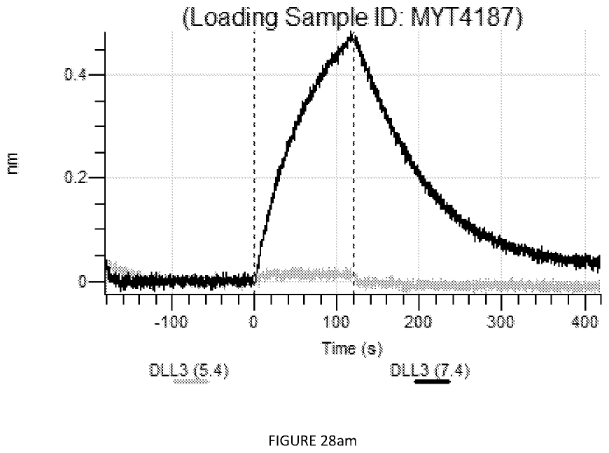

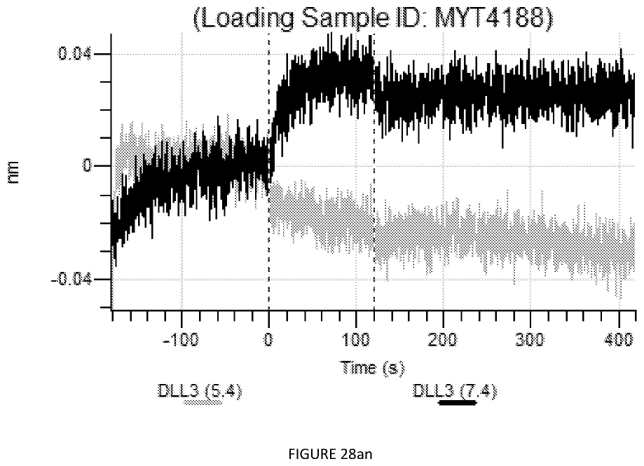

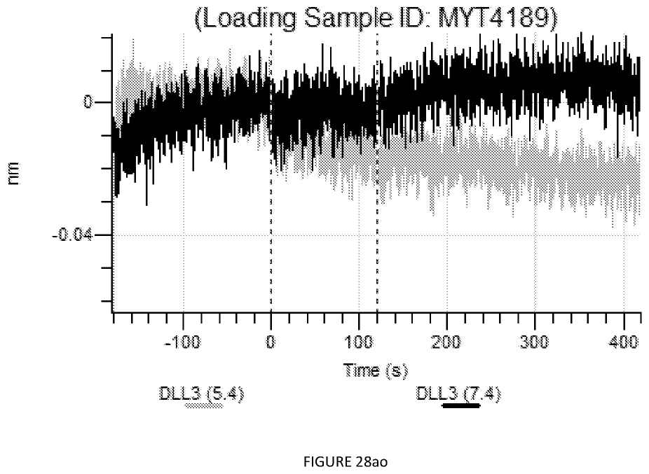

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.4 (U.S. Pat. No. 9,089,617 B2) (Heavy chain SEQ ID NO: 119, Light chain SEQ ID NO: 120) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy chain CDRs that had been previously selected for further analysis in Example 14 were systematically combined two or more at a time (MYT3399-MYT3424 and MYT4175-MYT4189). In cases where the starting CDR residue was a histidine, it was mutated to an alanine. Antibody variants with two or more histidine or alanine mutations in the heavy chain CDRs were generated by co-transfection of Expi293 cells with a) one heavy chain combinations sequence variant, and b) the corresponding starting ABPC light chain using methods known to the art. After allowing for four days of protein expression, cell culture supernatants were collected, quantified by SDS-PAGE analysis (FIG. 27), and the pH dependence of the variant was evaluated using biolayer interferometry (BLI) on an Octet RED 384 instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain combinations variants that showed either enhanced dissociation at pH 5.4 or reduced dissociation at pH 7.4 or both (as compared to the starting ABPC), as shown in FIG. 28, were selected for further analysis (e.g., MYT3399-MYT3424, MYT4175, MYT4176, MYT4177, MYT4178, MYT4180, MYT4181, MYT4182, MYT4184, MYT4185, and MYT4187).

### Example 17. Construction and Screening of pH-Engineered DLL3 ABPCs

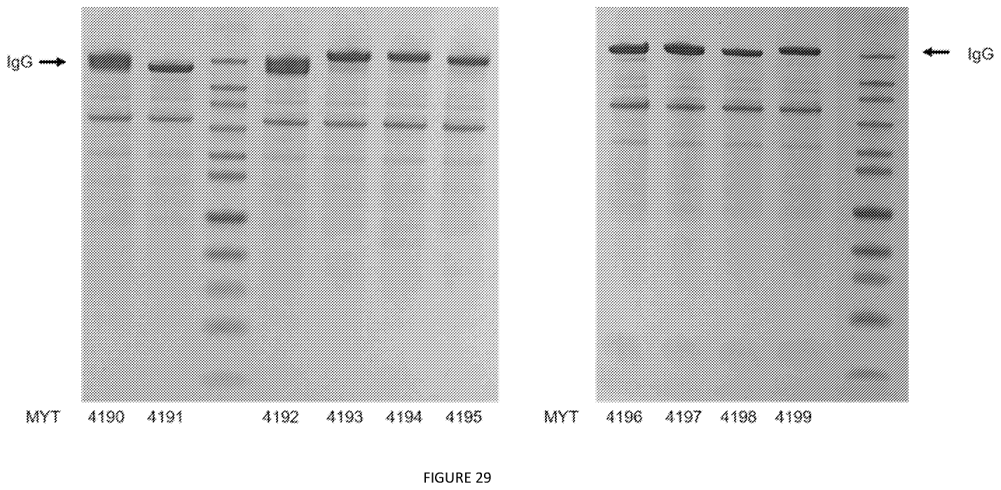

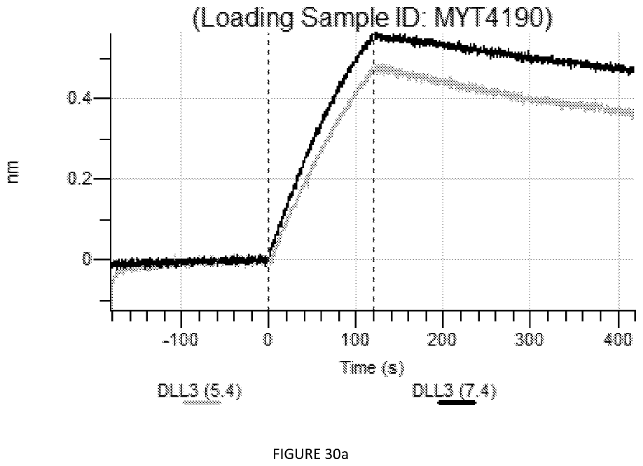

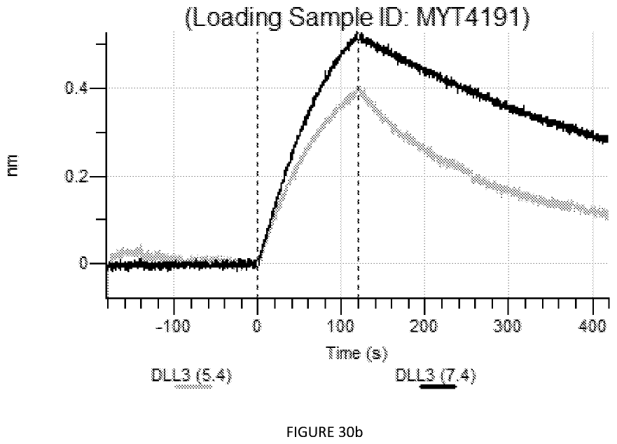

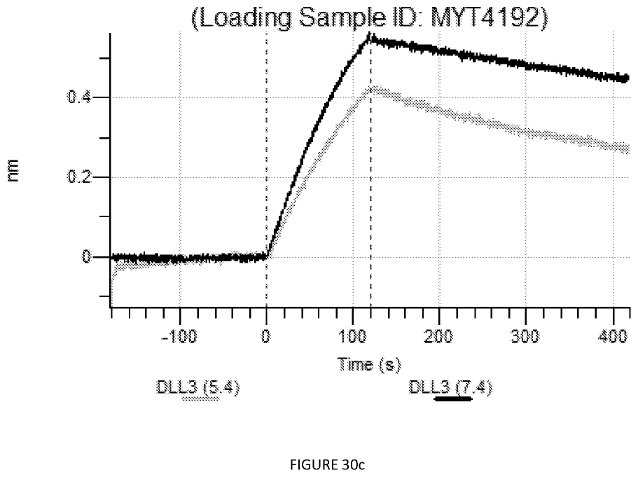

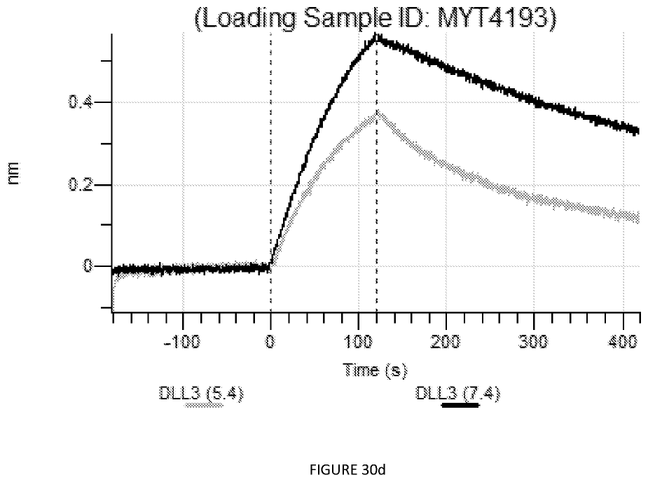

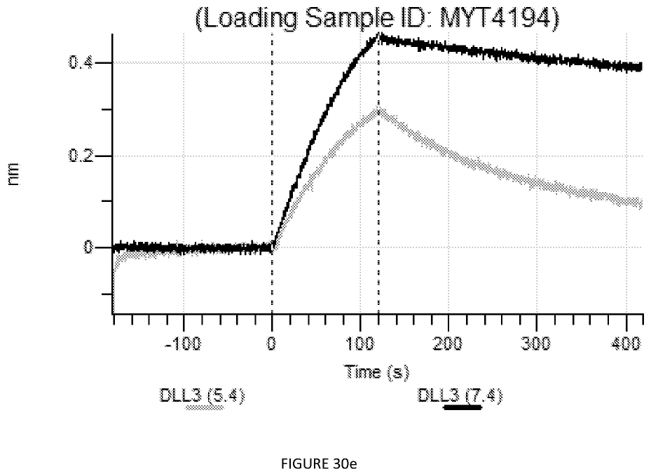

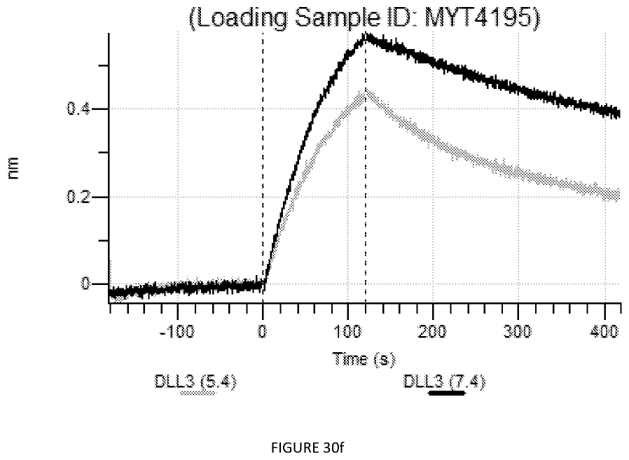

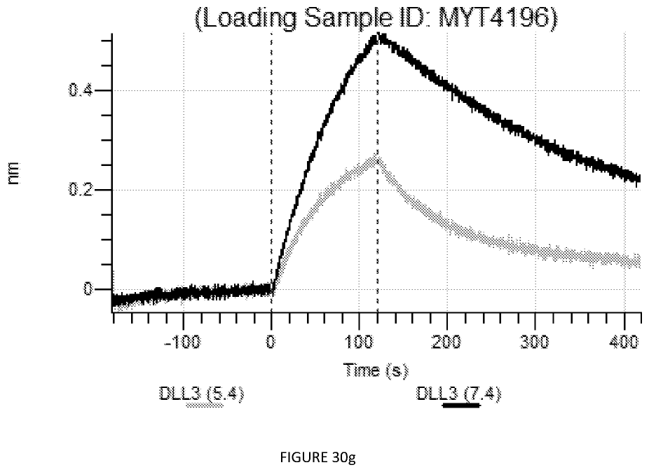

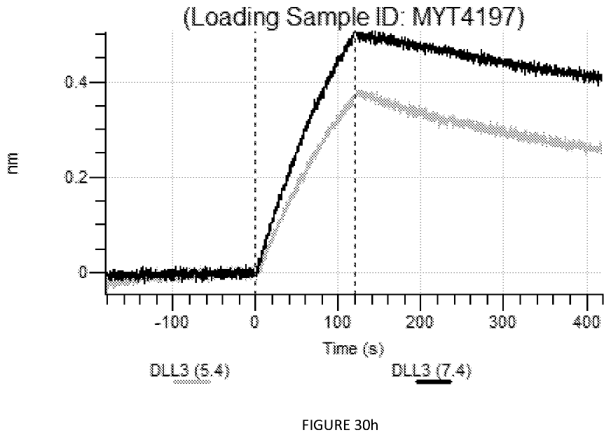

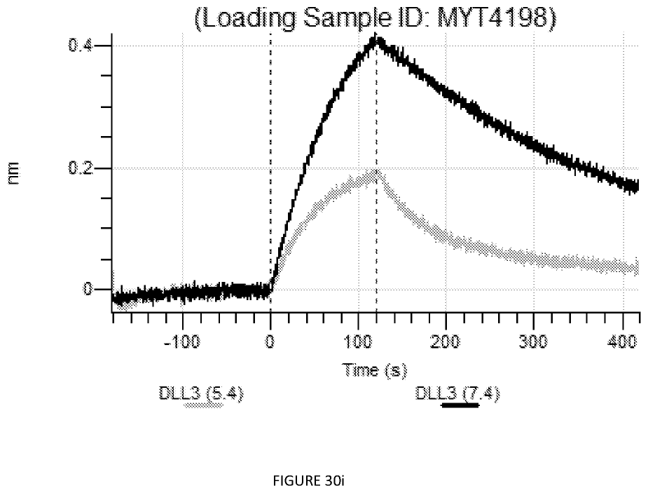

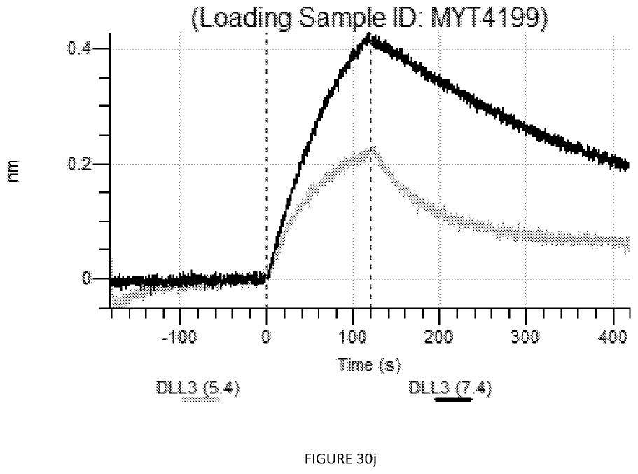

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.4 (U.S. Pat. No. 9,089,617 B2) (Heavy chain SEQ ID NO: 119, Light chain SEQ ID NO: 120) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the light chain CDRs that had been previously selected for further analysis in Example 15 were systematically combined two or more at a time (MYT4190-4199). In cases where the starting CDR residue was a histidine, it was mutated to an alanine. Antibody variants with two or more histidine or alanine mutations in the light chain CDRs were generated by co-transfection of Expi293 cells with a) one light chain combinations sequence variant, and b) the corresponding starting ABPC heavy chain using methods known to the art. After allowing for four days of protein expression, cell culture supernatants were collected, quantified by SDS-PAGE analysis (FIG. 29), and the pH dependence of the variant was evaluated using biolayer interferometry (BLI) on an Octet RED 384 or an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain combinations variants that showed either enhanced dissociation at pH 5.4 or reduced dissociation at pH 7.4 or both (as compared to the starting ABPC), as shown in FIG. 30 were selected for further analysis (MYT4190-4199).

### Example 18. Construction and Screening of pH-Engineered DLL3 ABPCs

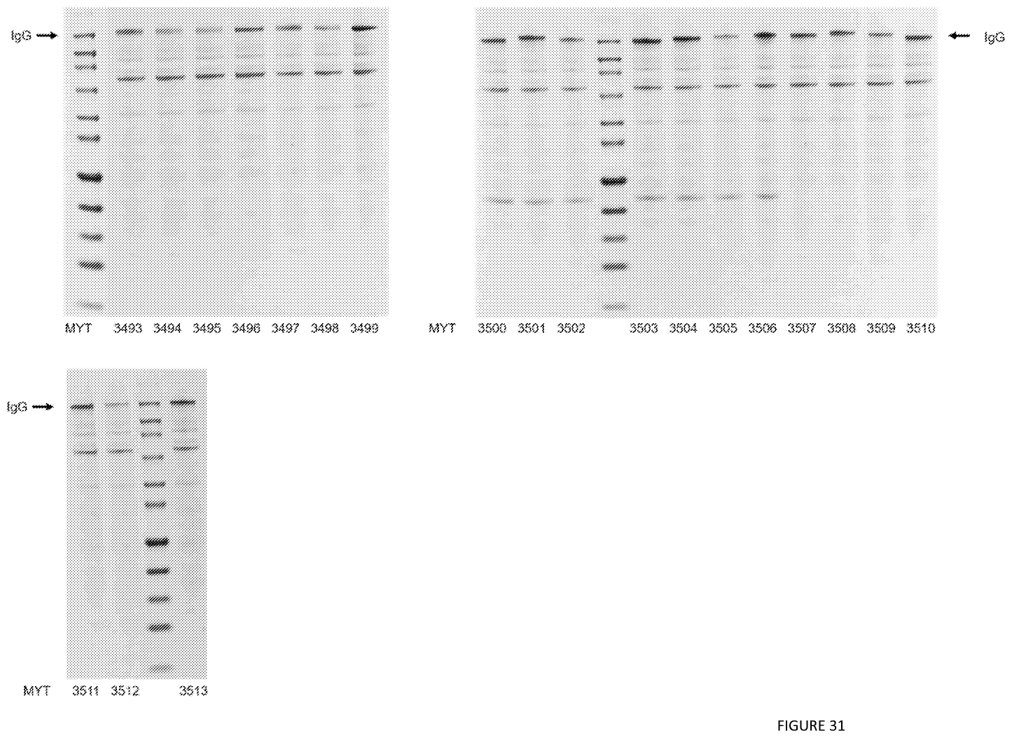

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.4 (U.S. Pat. No. 9,089,617 B2) (Heavy chain SEQ ID NO: 119, Light chain SEQ ID NO: 120) was selected as a DLL3-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy and light chain CDRs that had been previously selected for further analysis in Examples 14-17 were systematically combined two or more at a time (MYT3493-3513). In cases where the starting CDR residue was a histidine, it was mutated to an alanine. Antibody variants with two or more histidine or alanine mutations were generated by co-transfection of Expi293 cells with a) one light chain sequence variant, and b) one heavy chain sequence variant using methods known to the art. After allowing for four days of protein expression, cell culture supernatants were collected, quantified by SDS-PAGE analysis (FIG. 31), and the pH dependence of the variant was evaluated using biolayer interferometry (BLI) on an Octet RED 384 or an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Paired heavy and light chain variants that showed either enhanced dissociation at pH 5.4 or reduced dissociation at pH 7.4 or both (as compared to the starting ABPC), as shown in FIG. 32, were selected for further analysis (MYT3493-MYT3513).

### Example 19. Construction and Screening of pH-Engineered DLL3 ABPCs

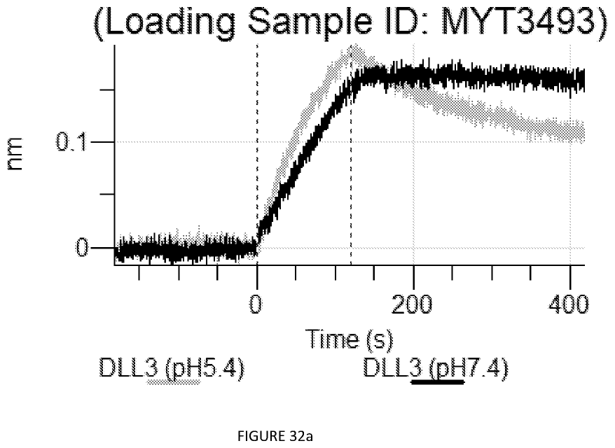

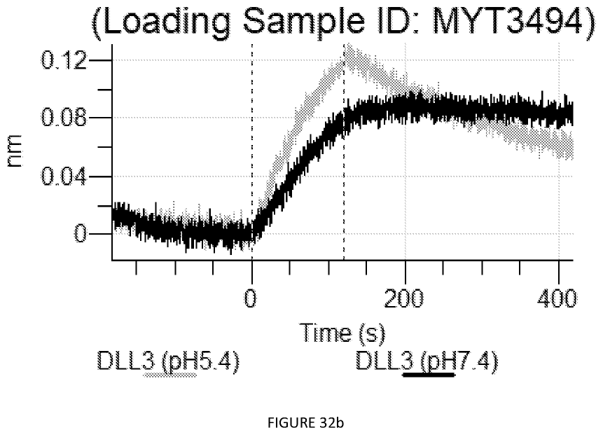

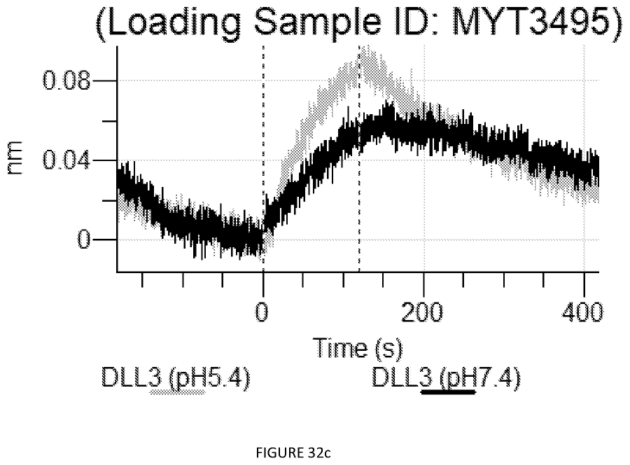

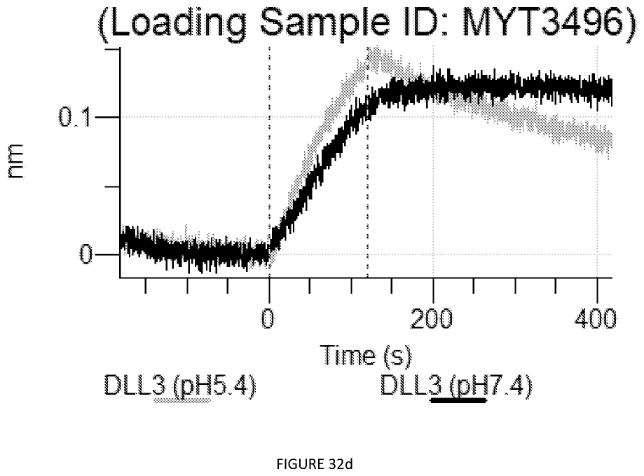

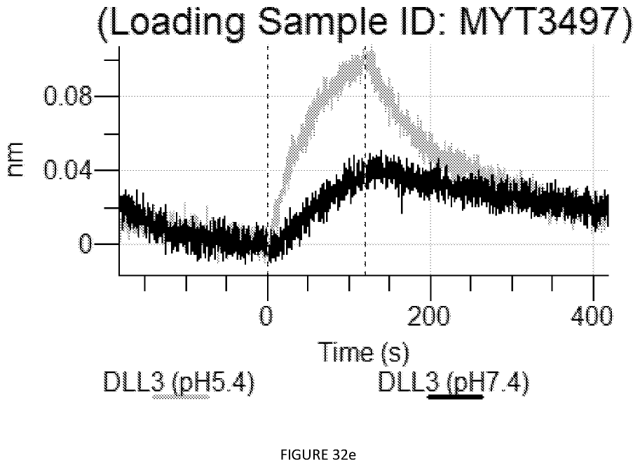

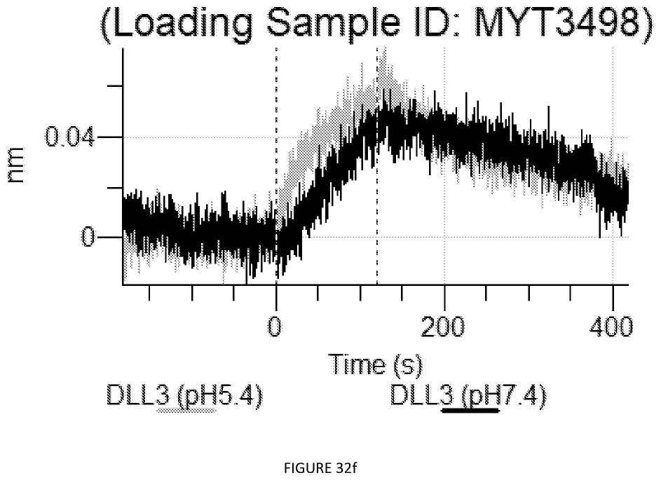

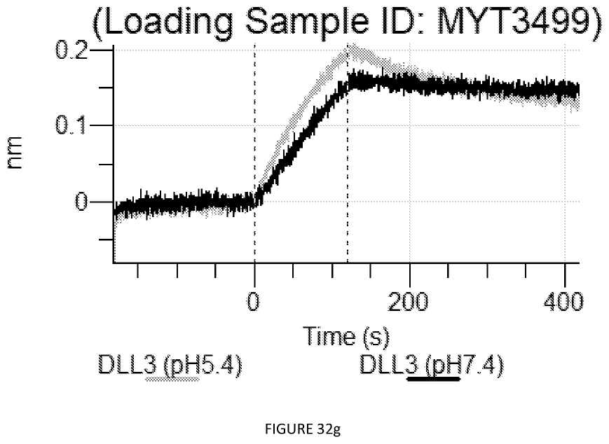

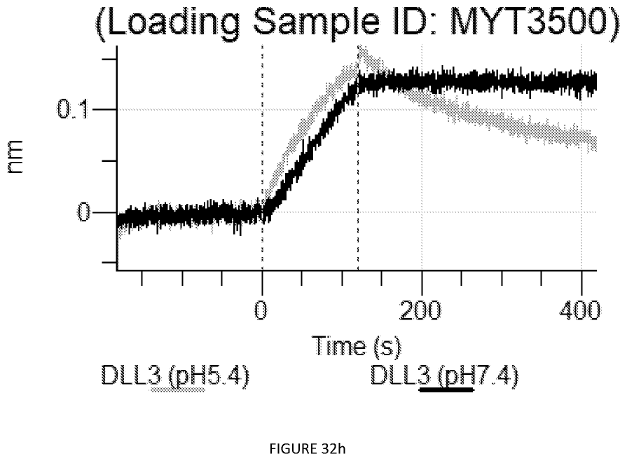

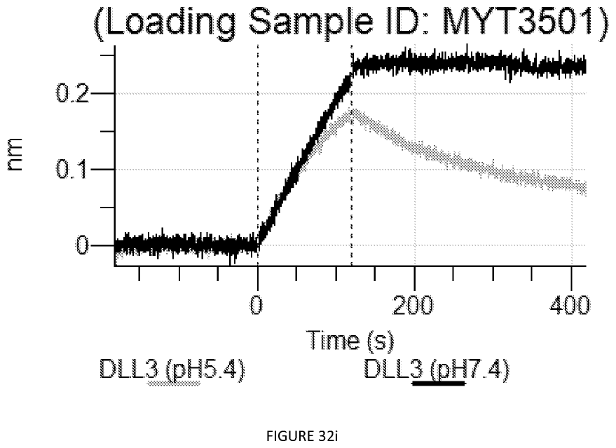

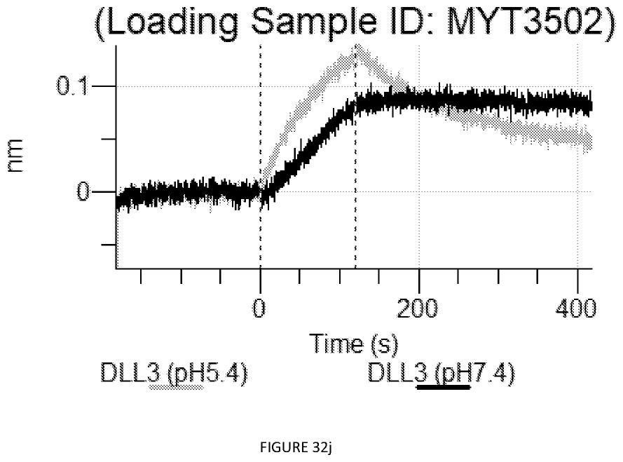

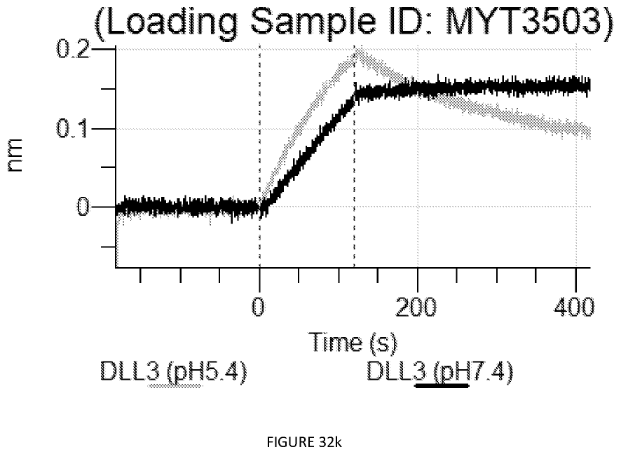

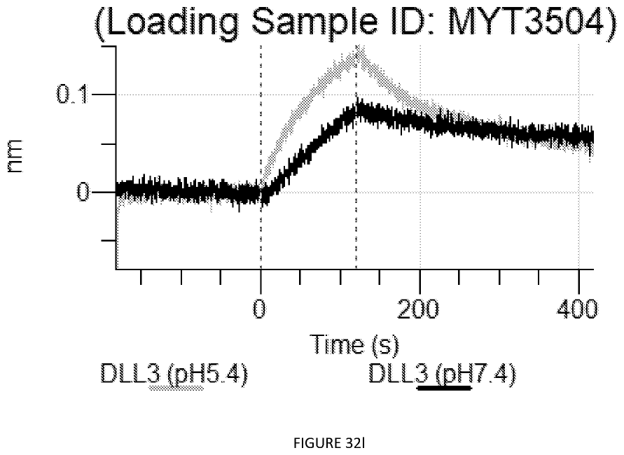

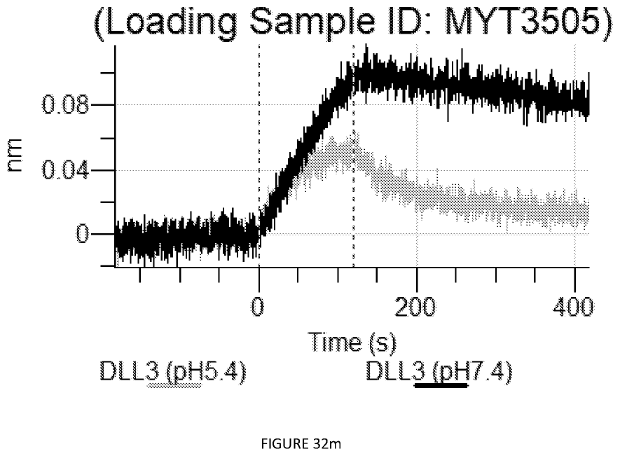

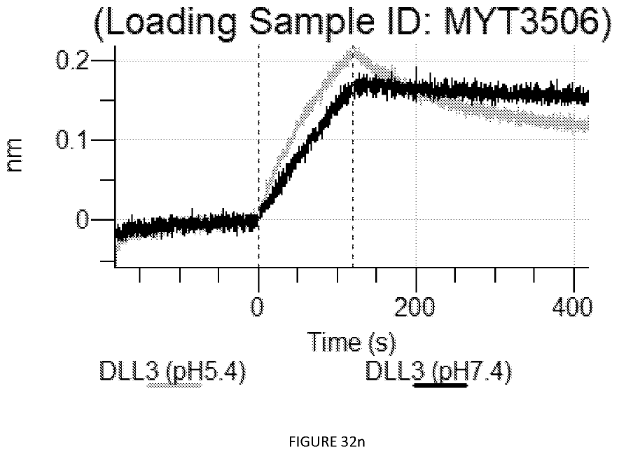

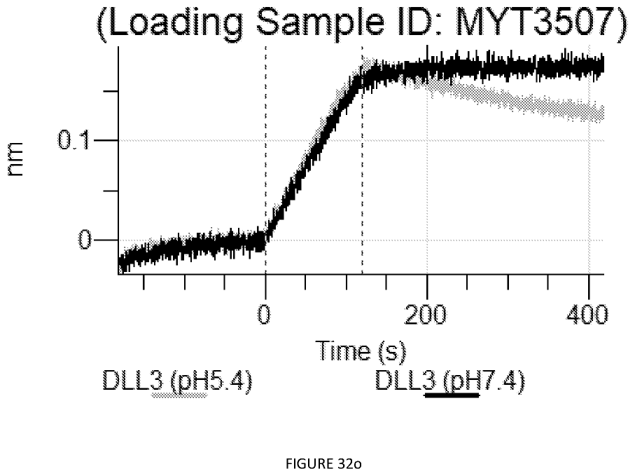

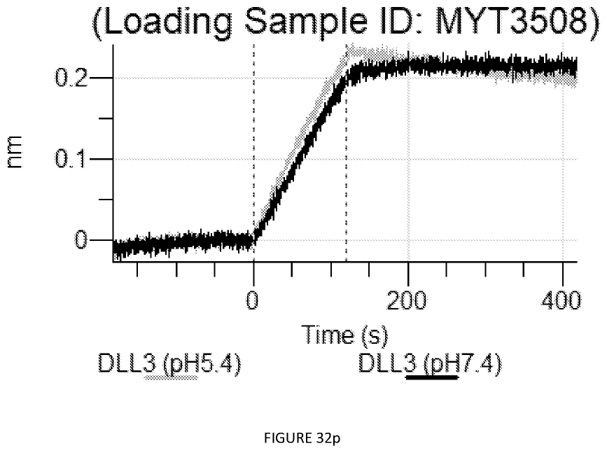

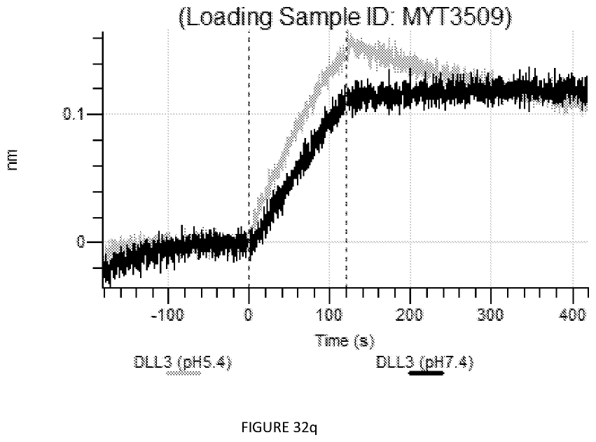

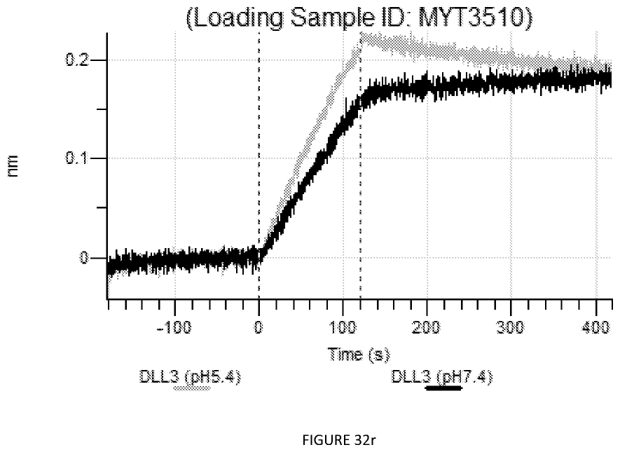

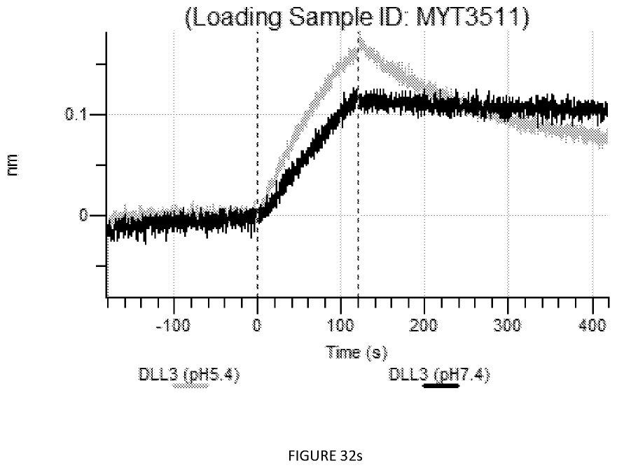

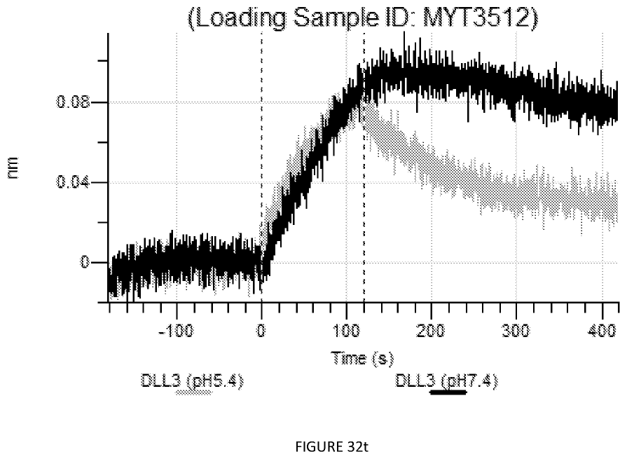

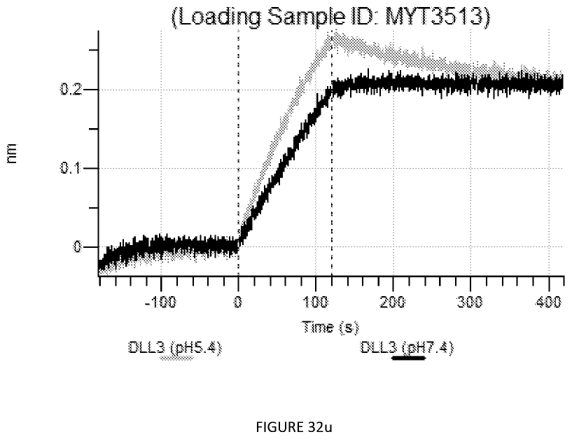

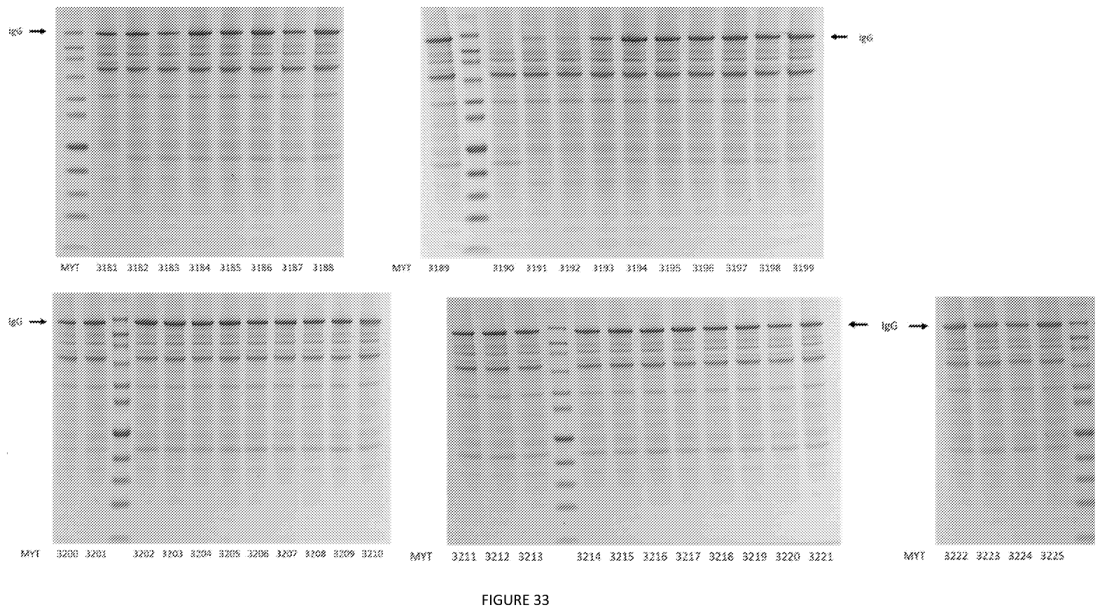

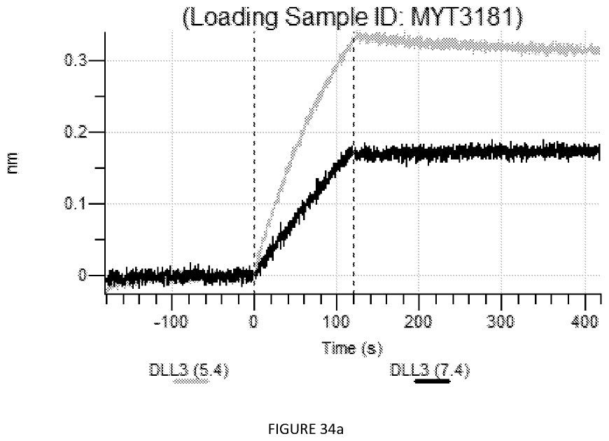

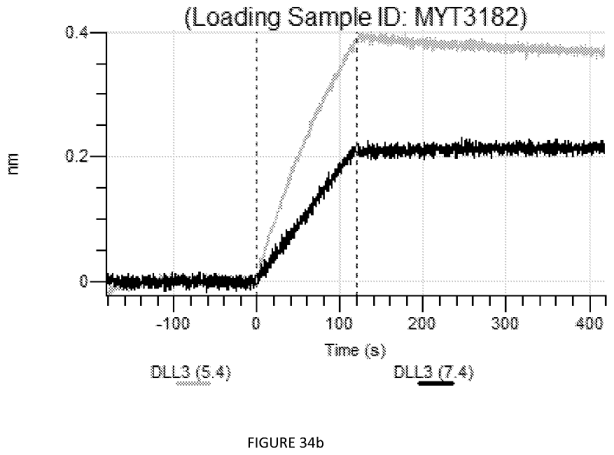

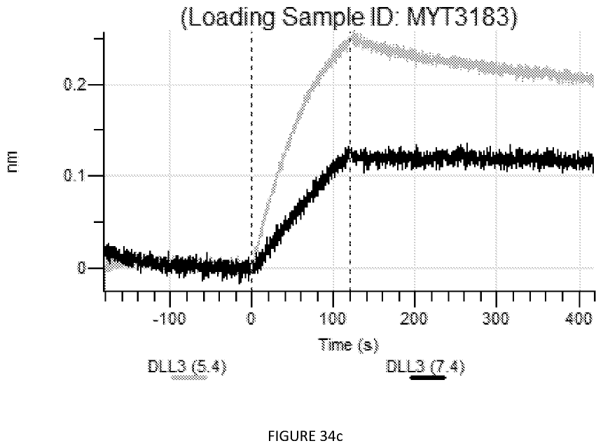

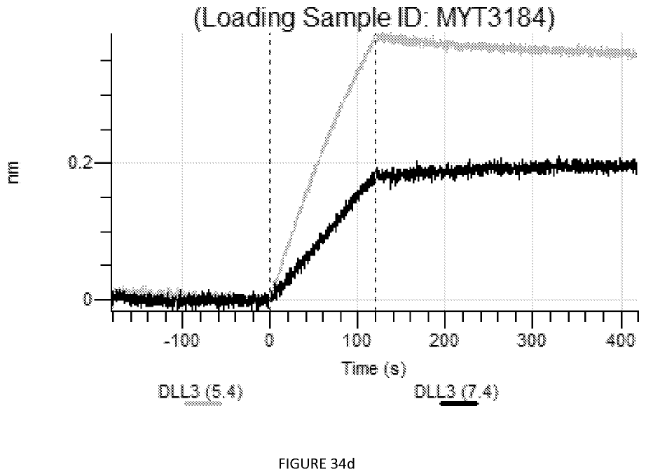

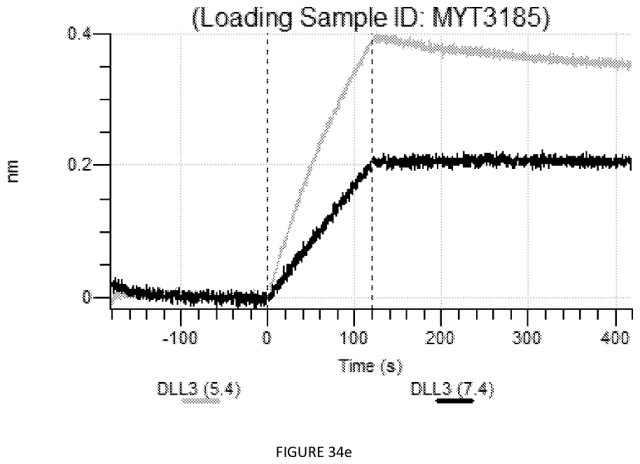

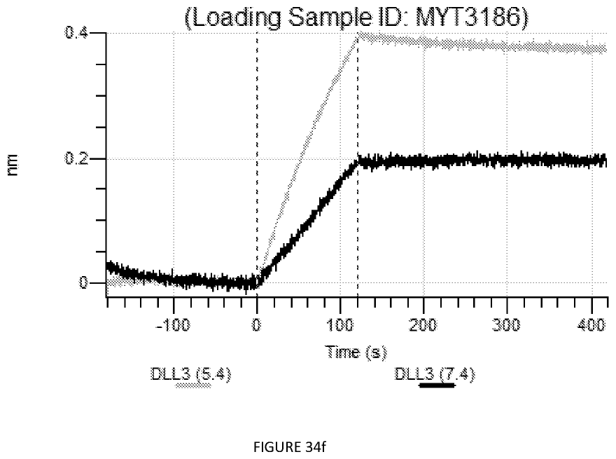

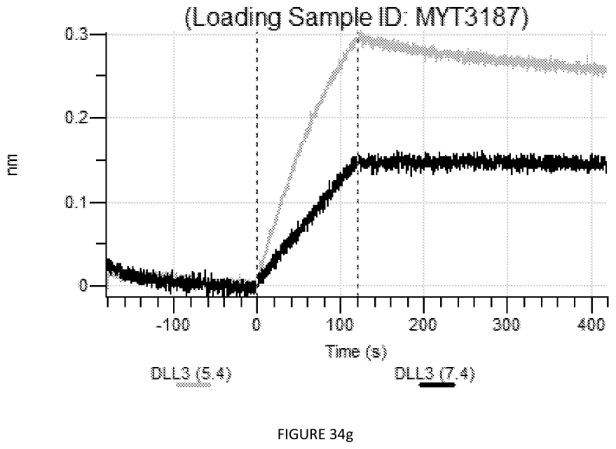

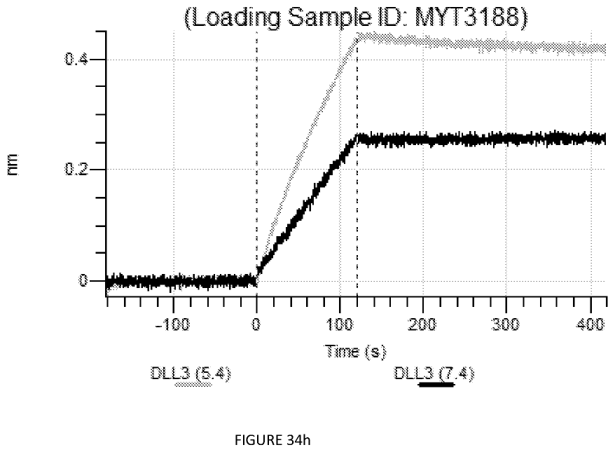

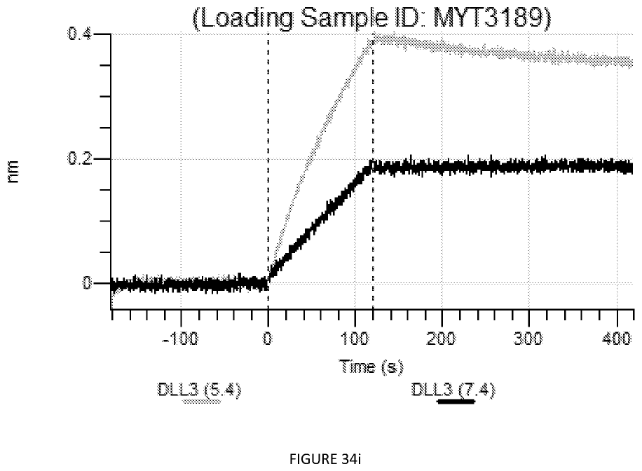

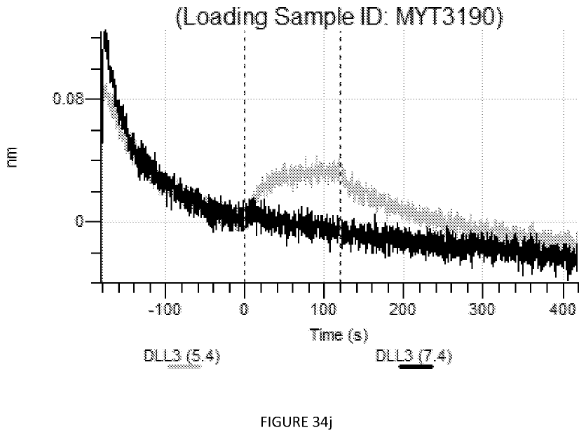

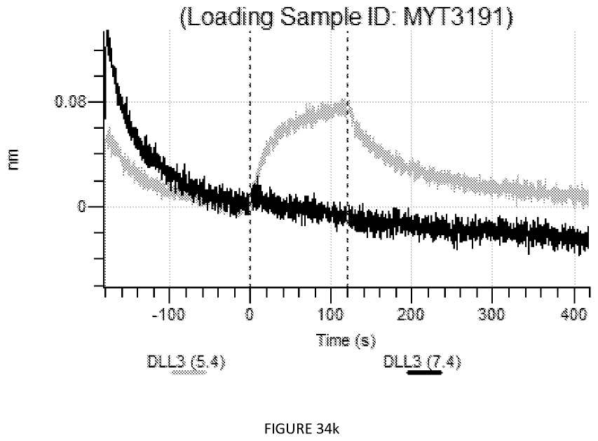

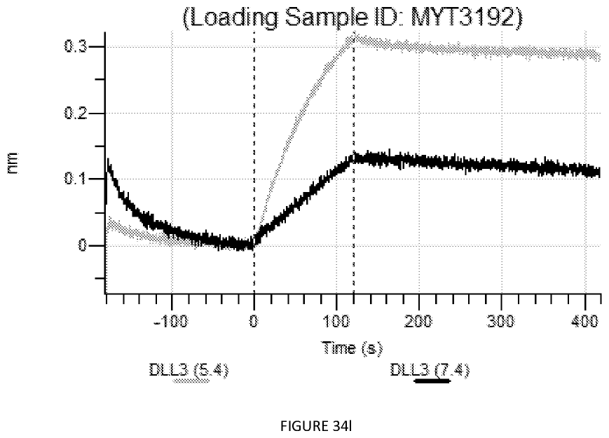

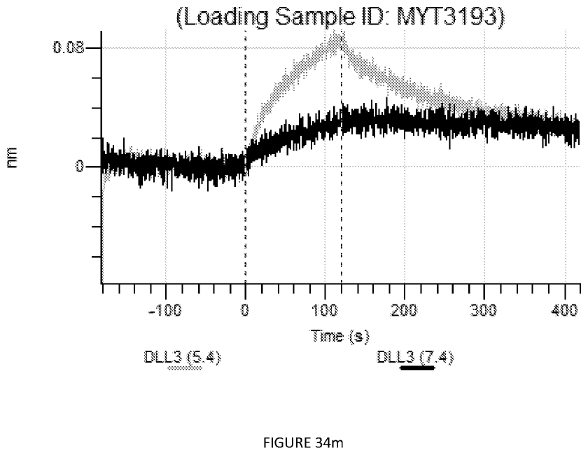

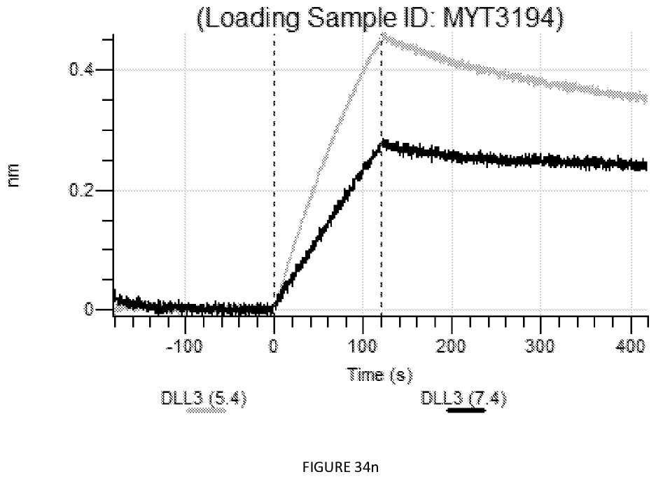

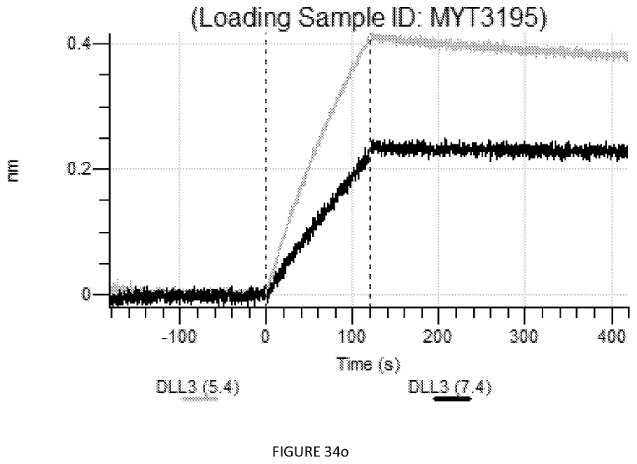

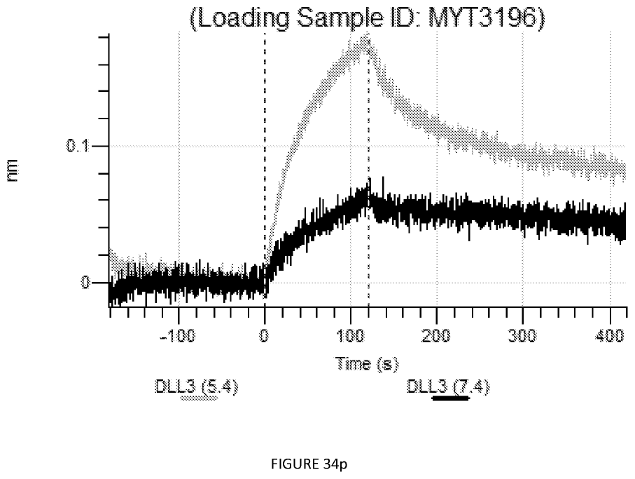

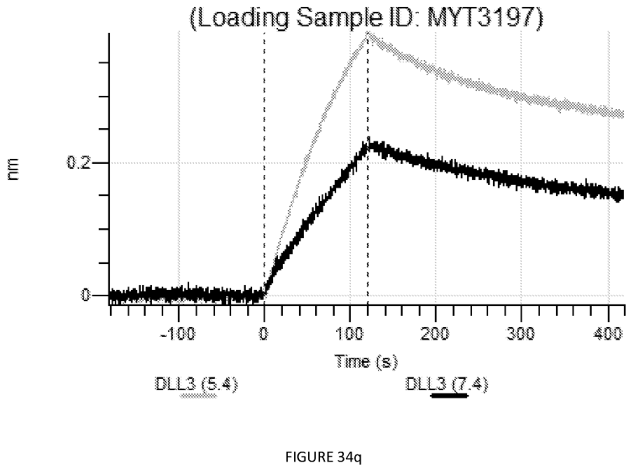

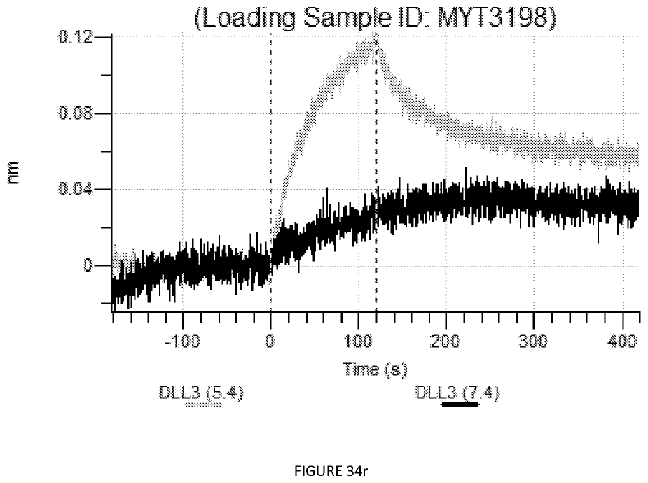

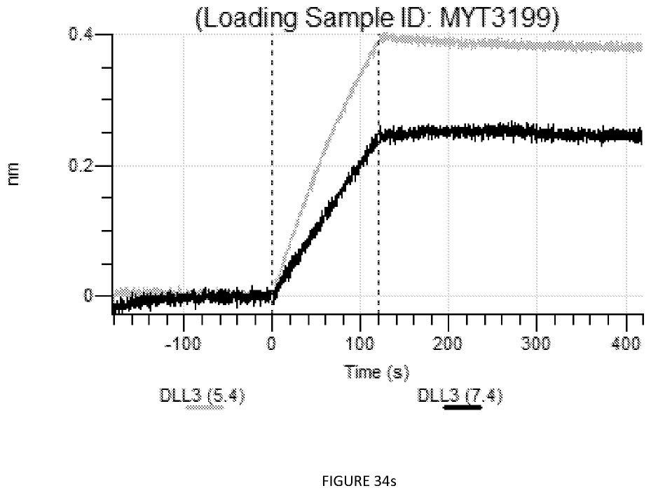

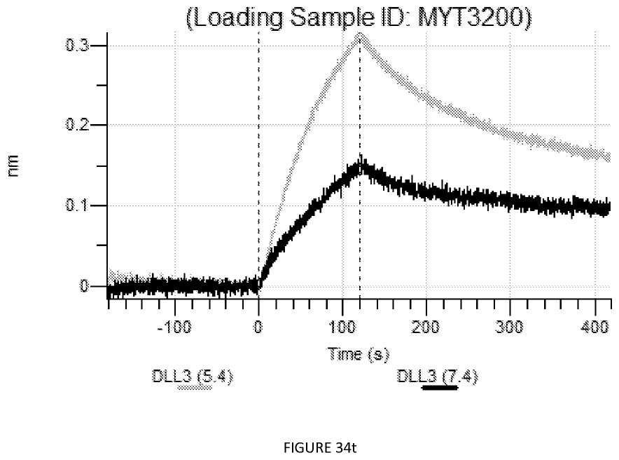

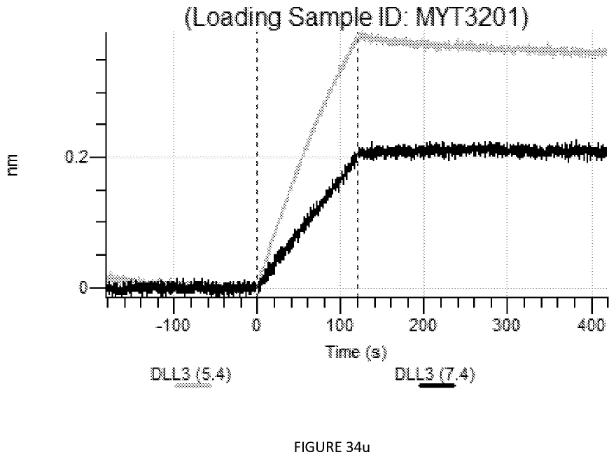

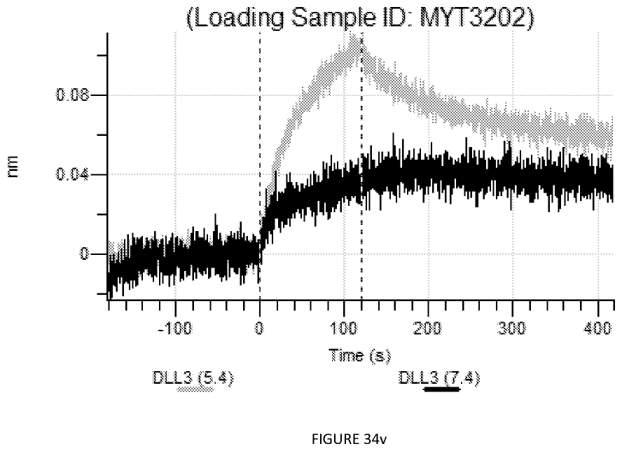

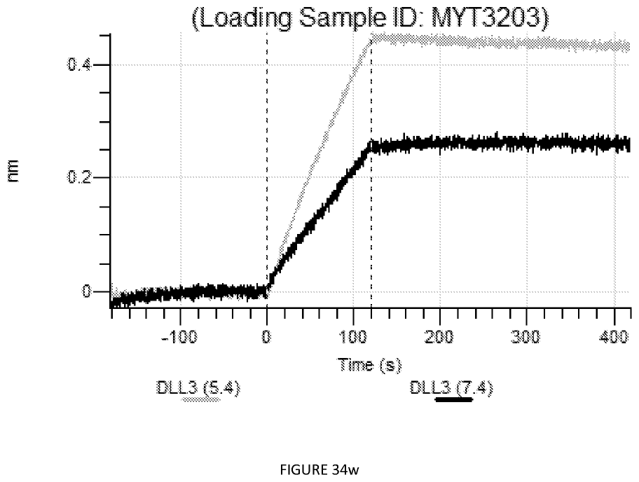

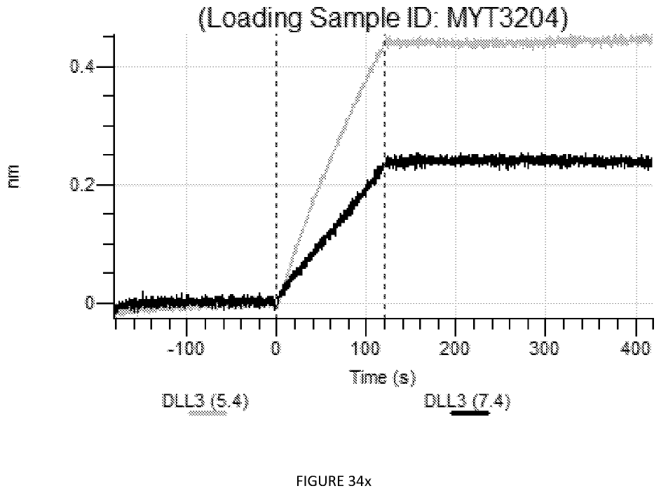

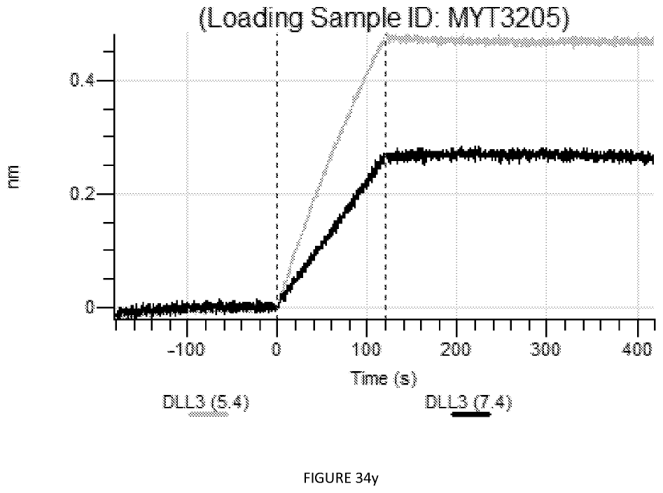

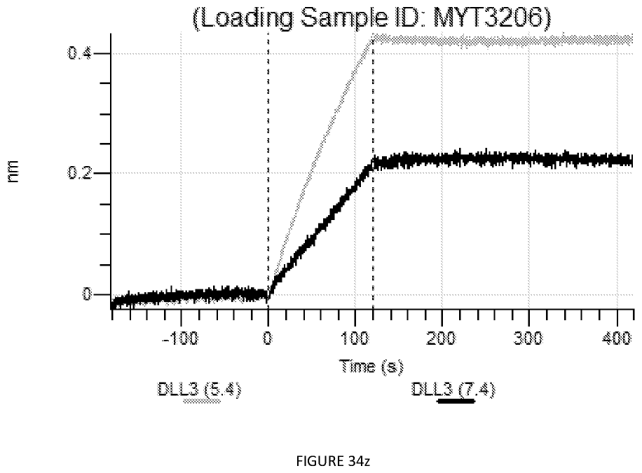

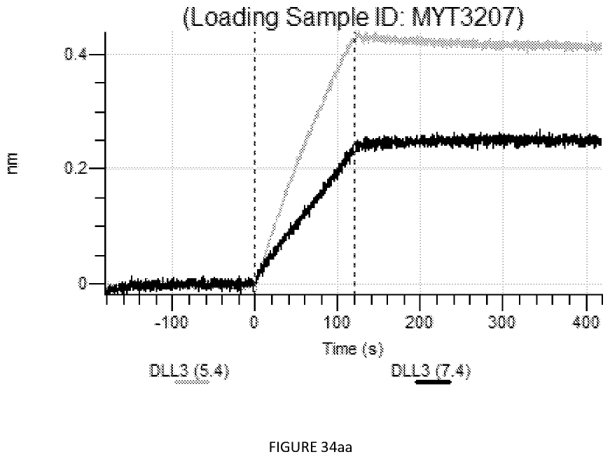

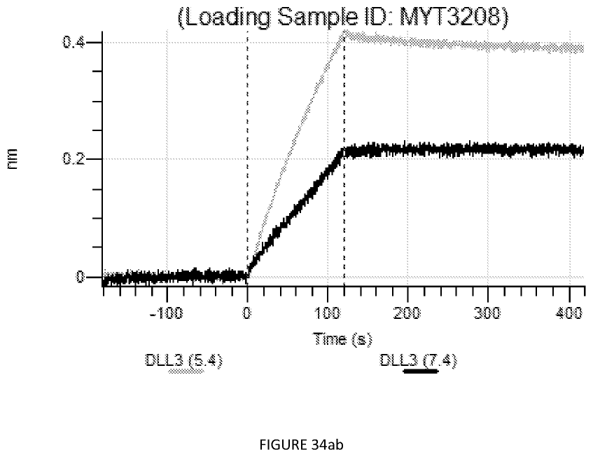

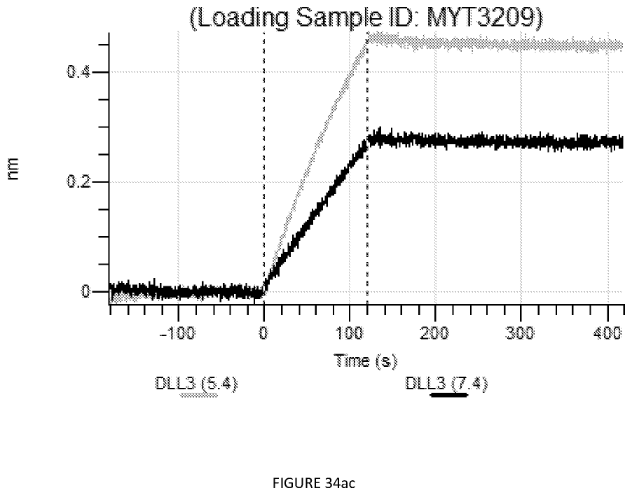

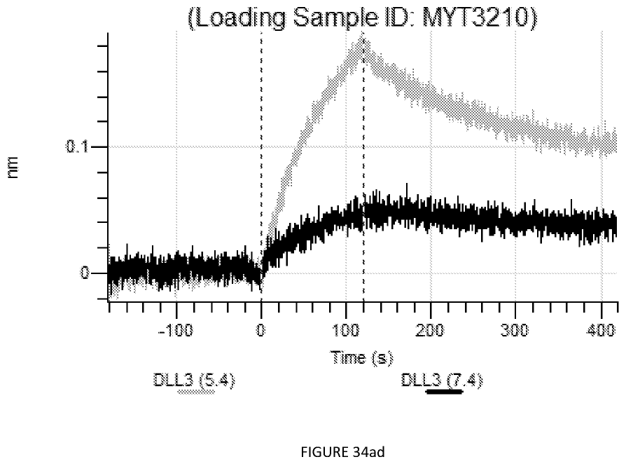

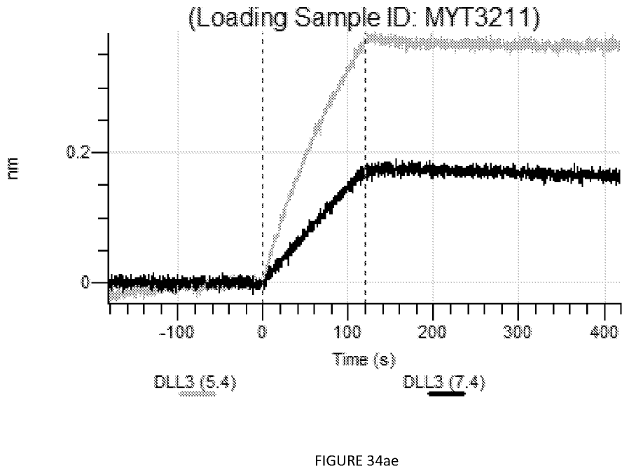

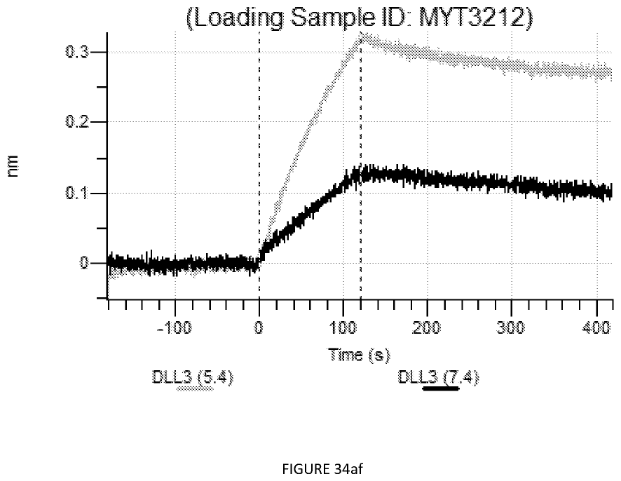

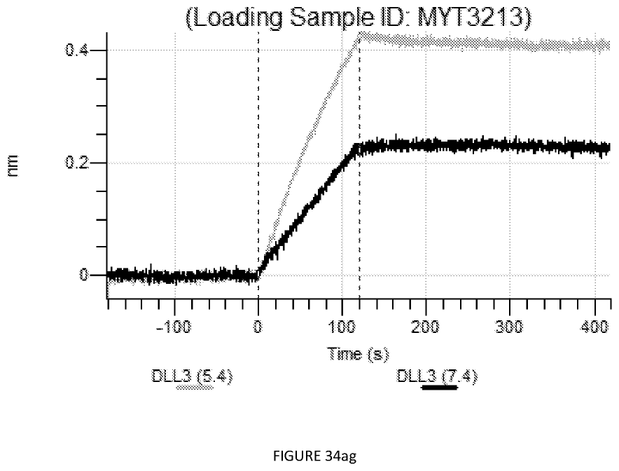

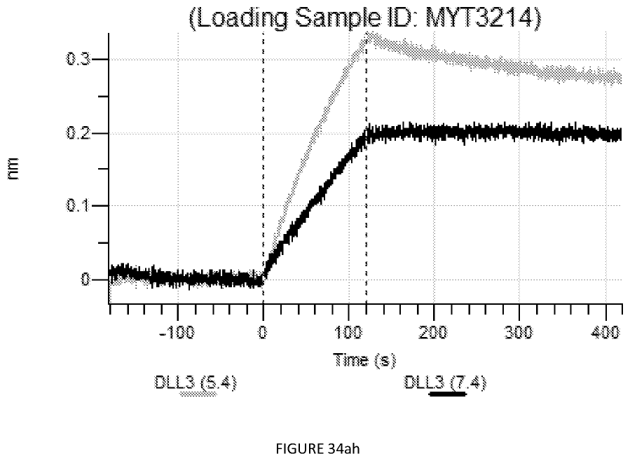

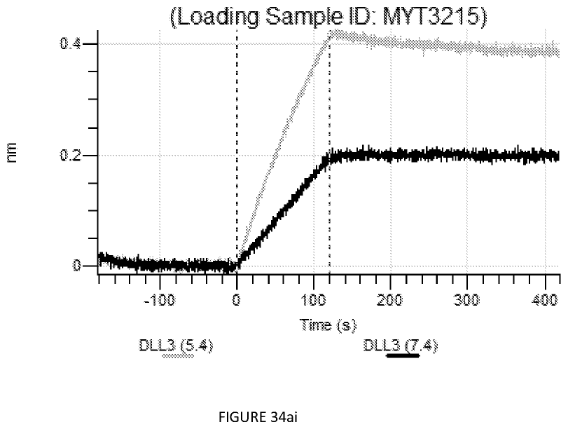

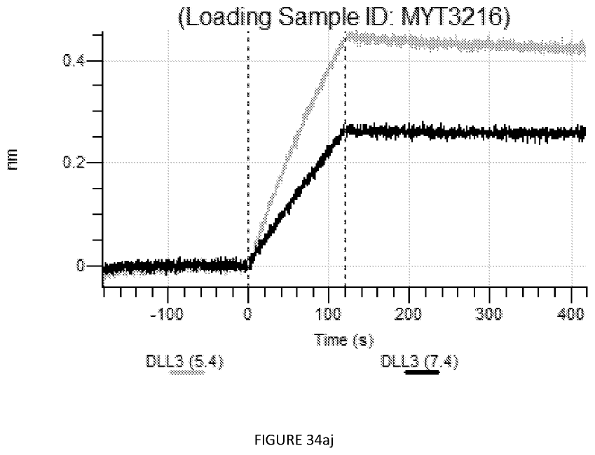

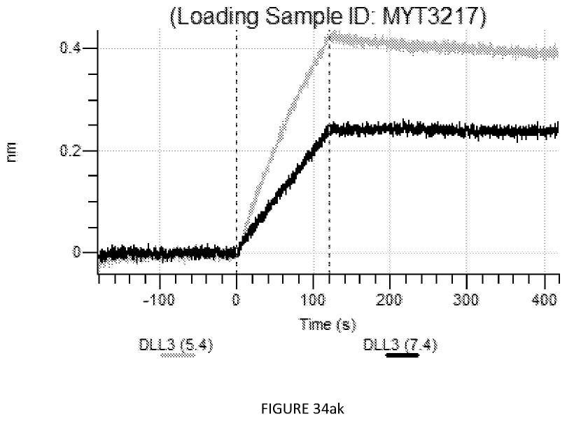

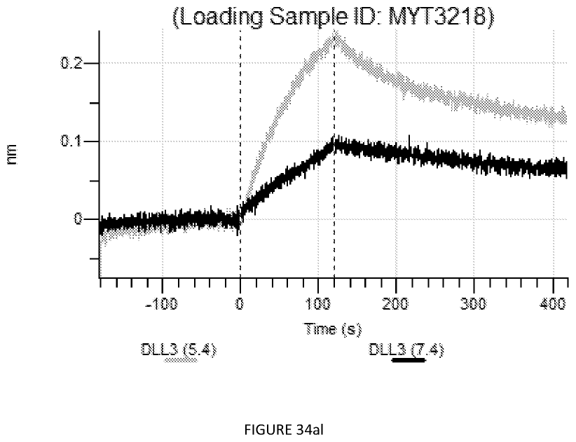

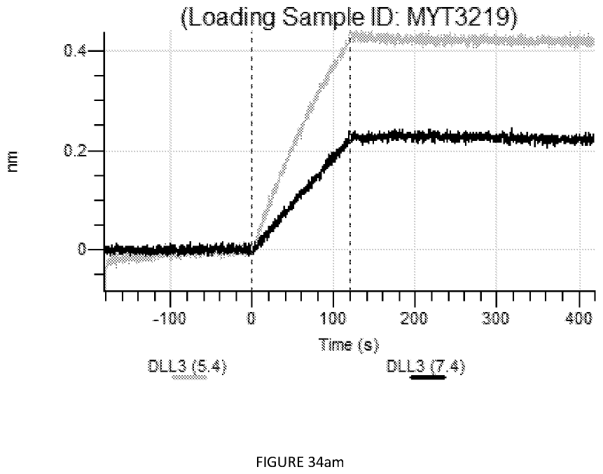

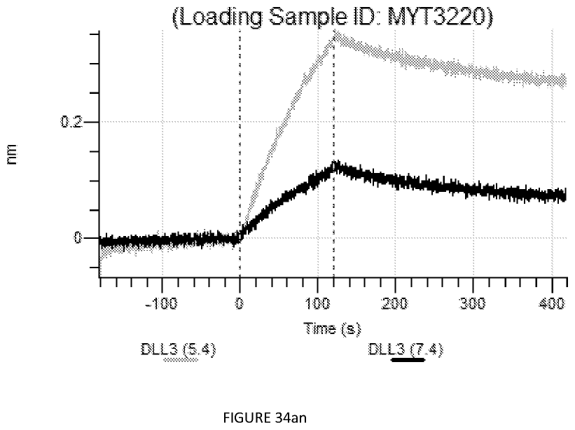

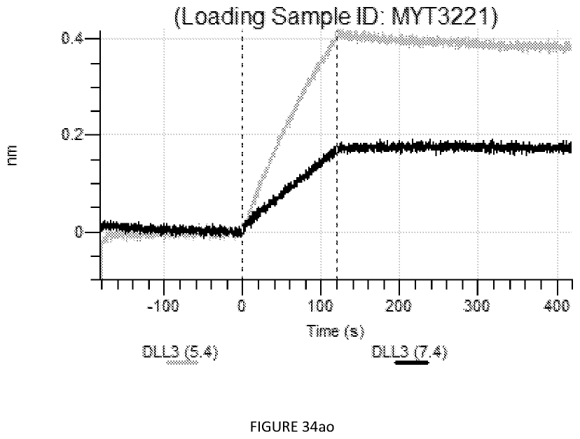

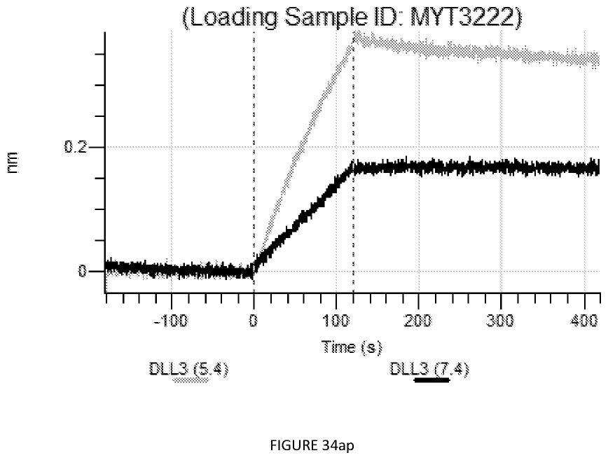

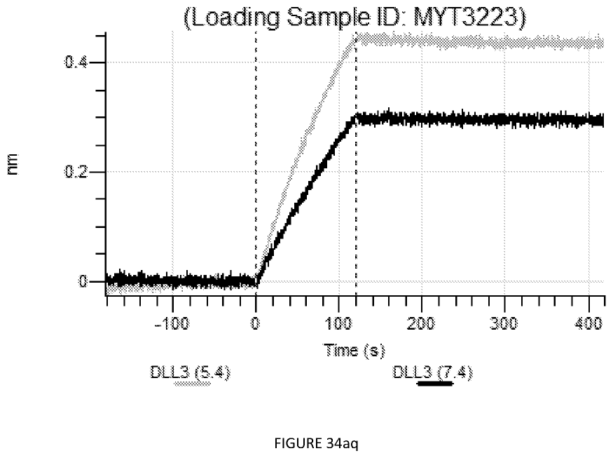

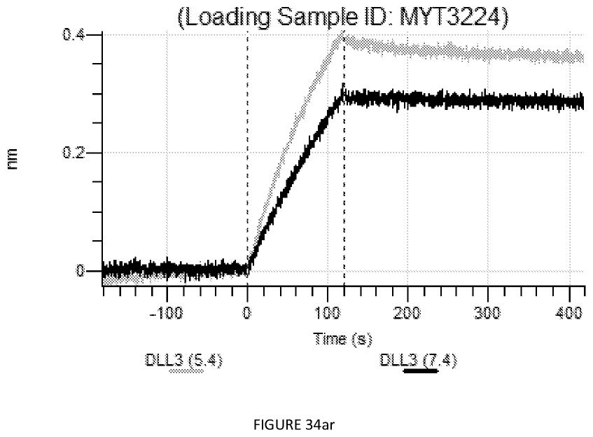

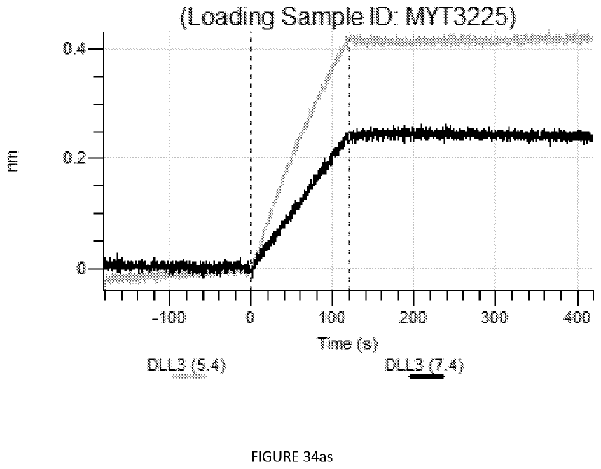

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.13 (U.S. Pat. No. 9,089,617 B2) (Heavy chain SEQ ID NO: 236, Light chain SEQ ID NO: 237) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3182-MYT3225). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 33) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 34, were selected for further analysis (MYT3183, MYT3185, MYT3187, MYT3189-MYT3194, MYT3196, MYT3197, MYT3198, MYT3200, MYT3202, MYT3210, MYT3212, MYT3214, MYT3218, MYT3220, and MYT3222). It was also noted that while no histidine or alanine mutations obliterated DLL3 binding, others were tolerated with little (e.g., less than 1-fold change in dissociation constant KD or dissociation rate) or no change in DLL3 binding kinetics (e.g., MYT3182, MYT3184, MYT3186, MYT3188, MYT3195, MYT3199, MYT3201, MYT3203, MYT3204, MYT3205, MYT3206, MYT3207, MYT3208, MYT3209, MYT3211, MYT3213, MYT3215, MYT3216, MYT3217, MYT3219, MYT3221, MYT3223, MYT3224, and MYT3225). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 20. Construction and Screening of pH-Engineered DLL3 ABPCs

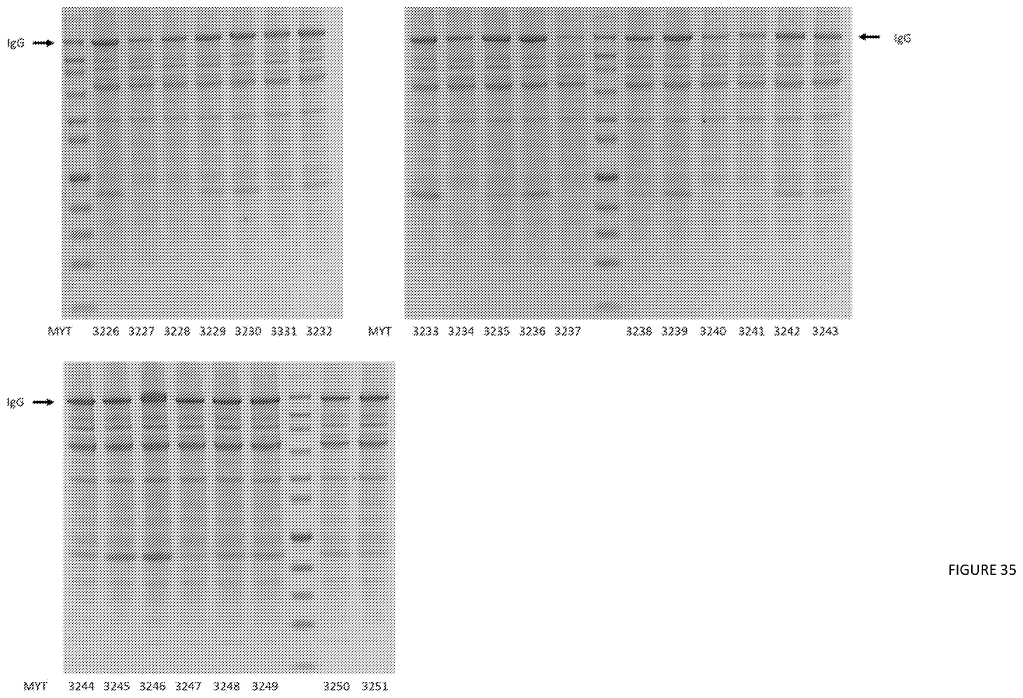

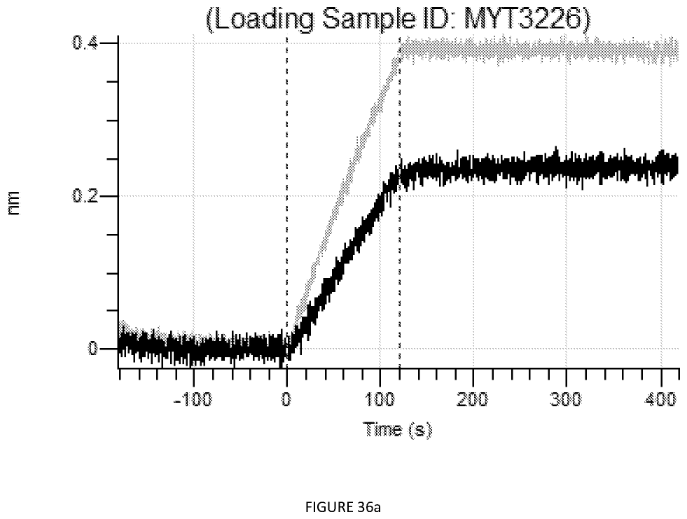

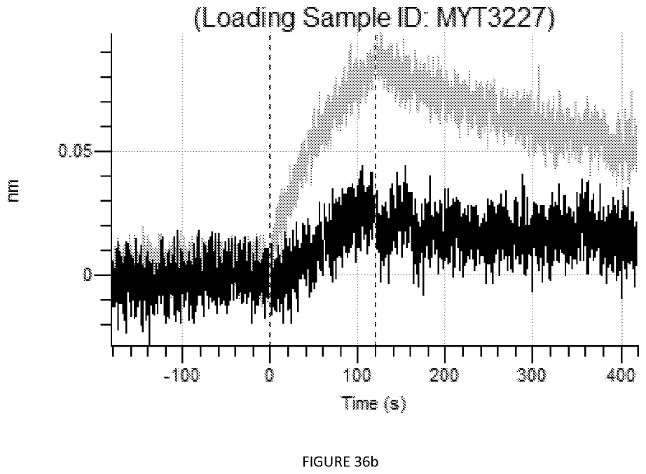

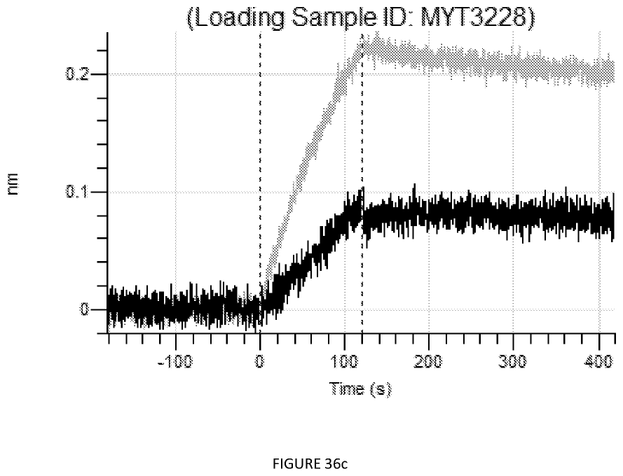

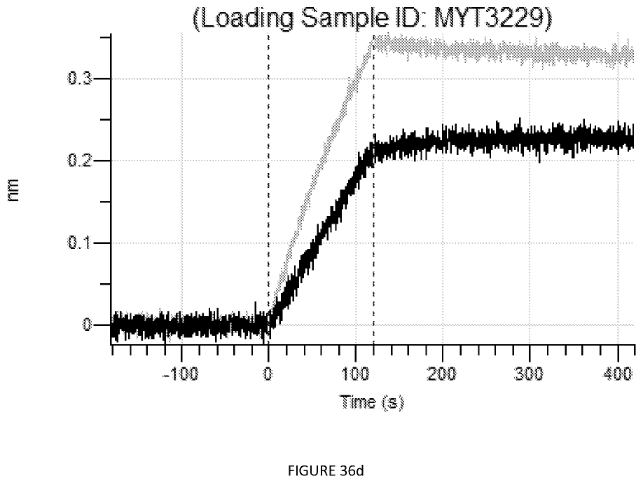

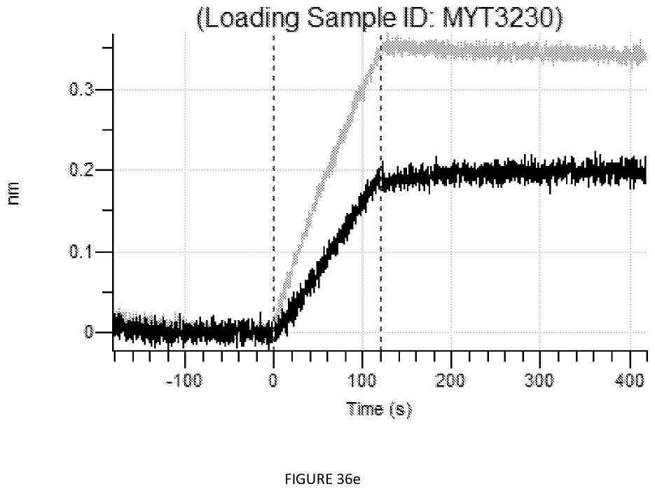

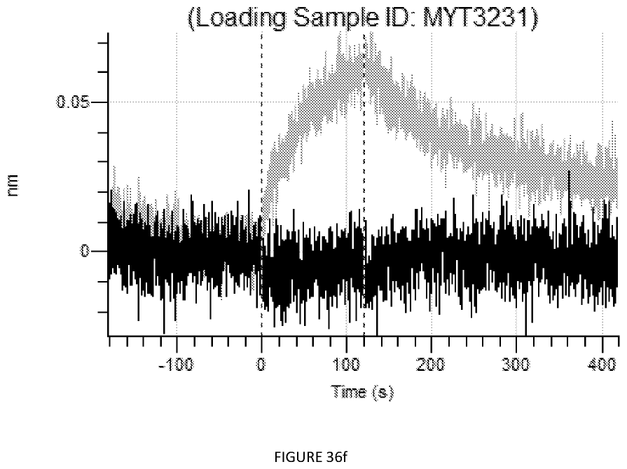

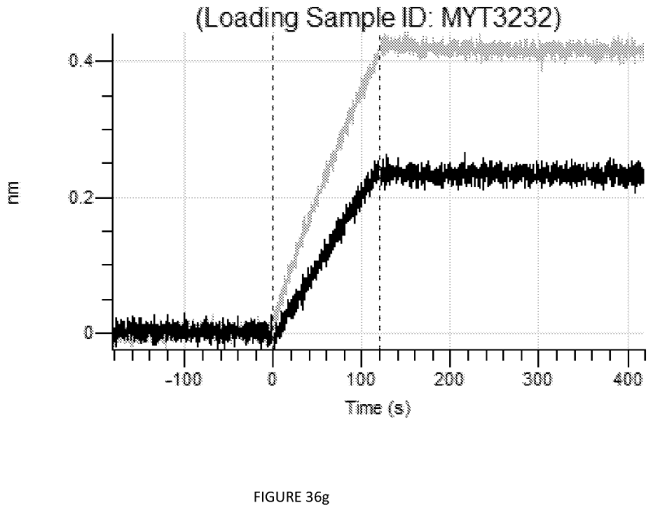

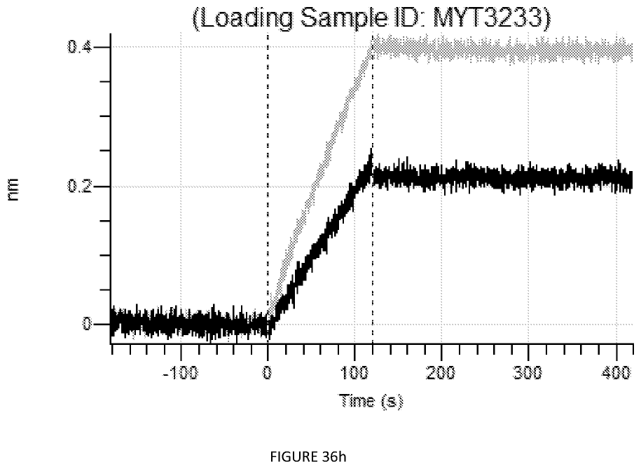

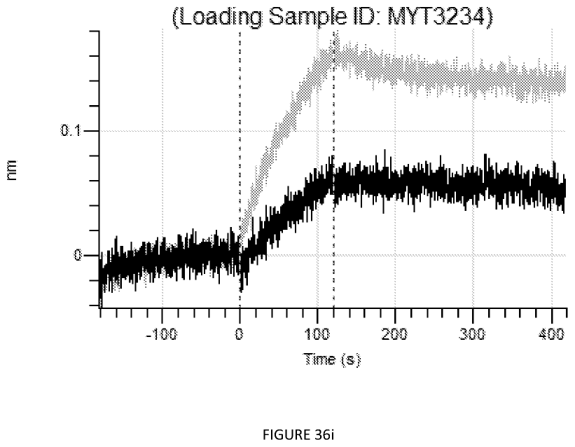

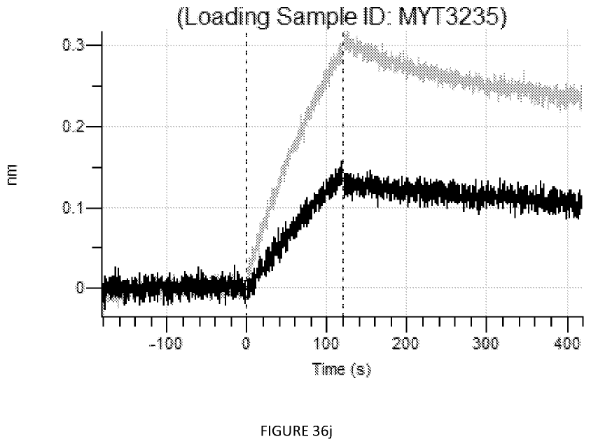

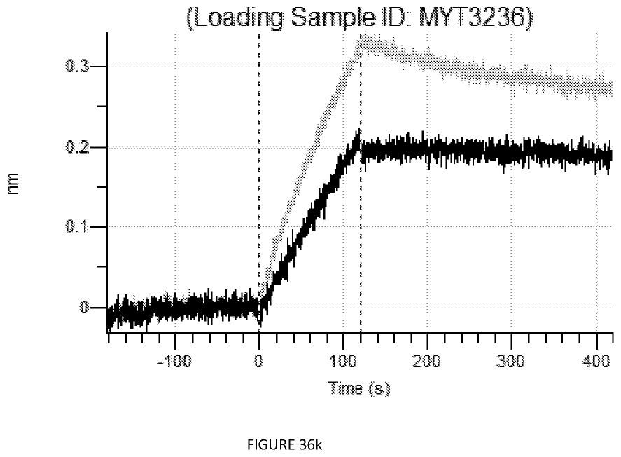

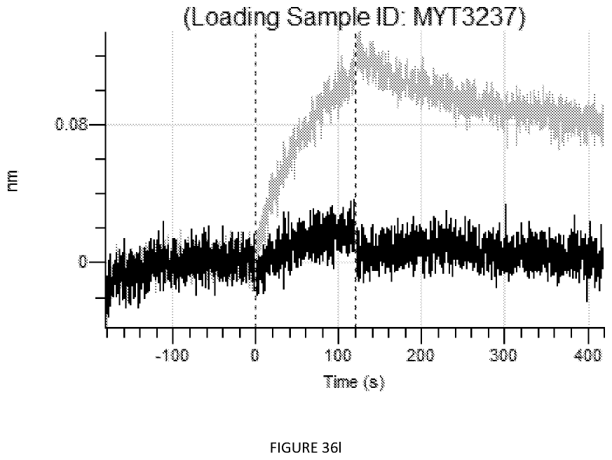

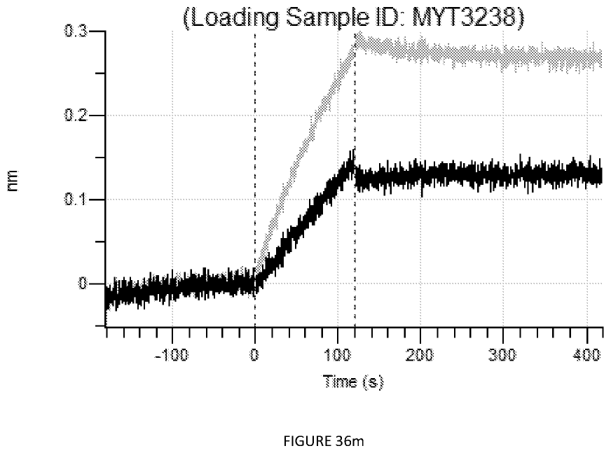

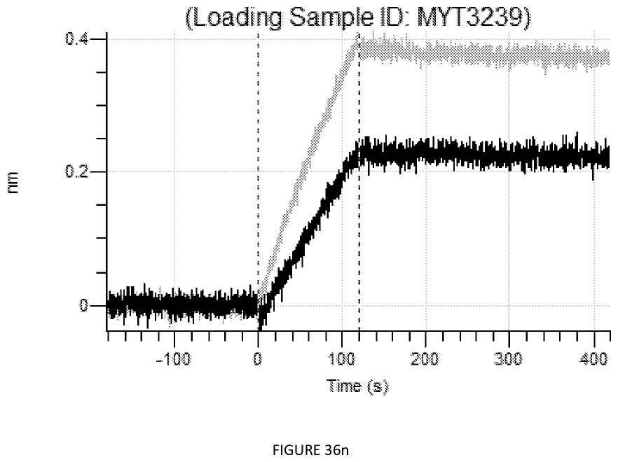

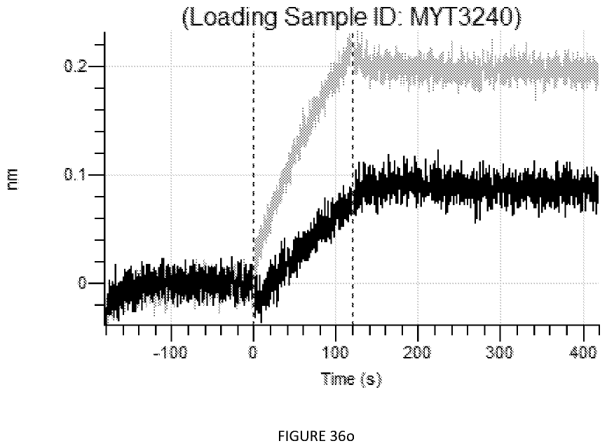

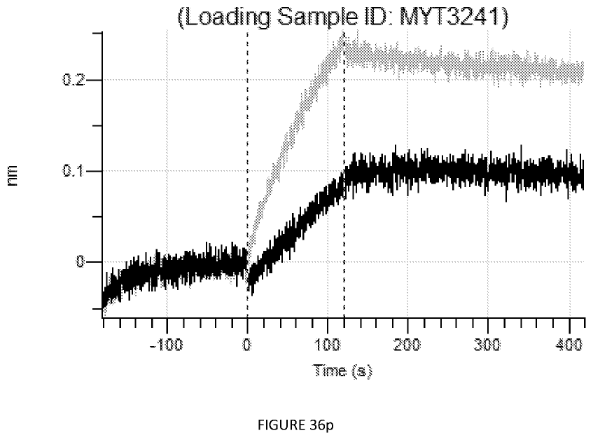

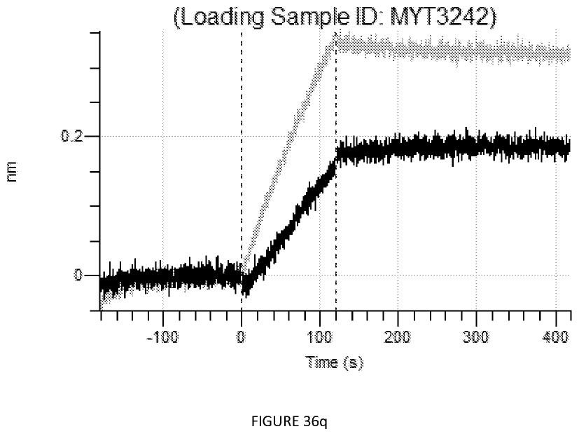

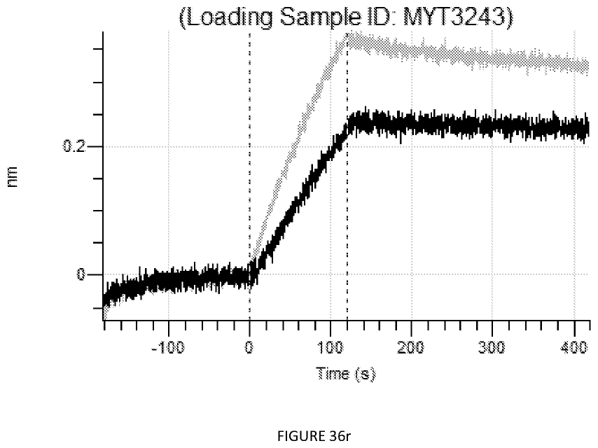

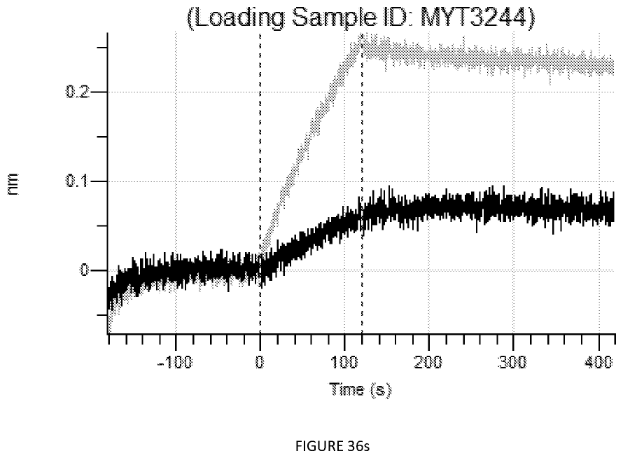

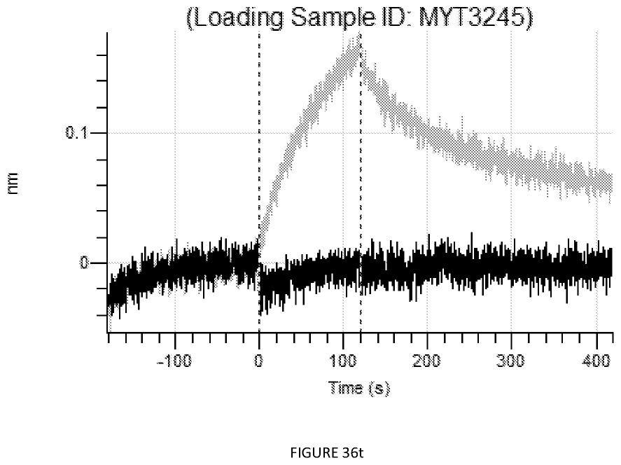

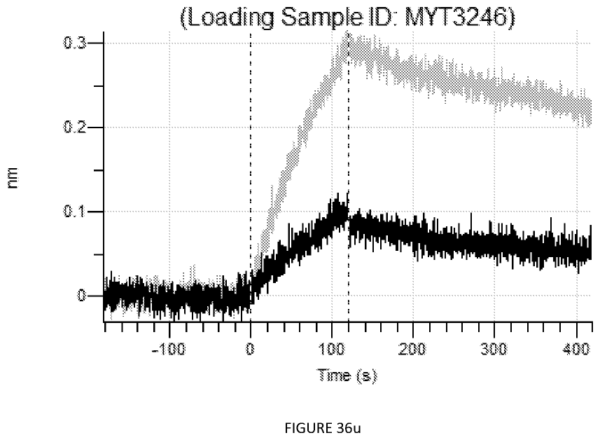

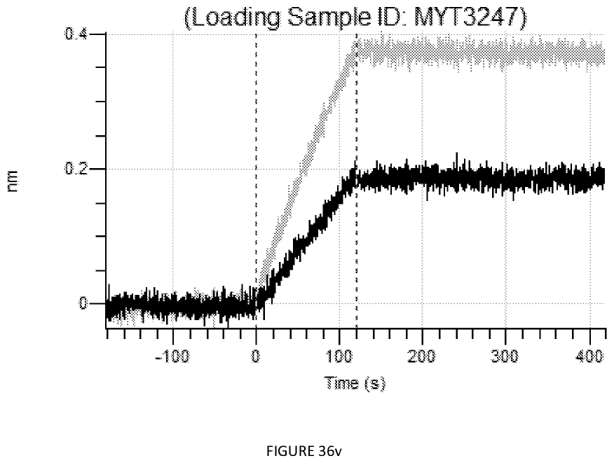

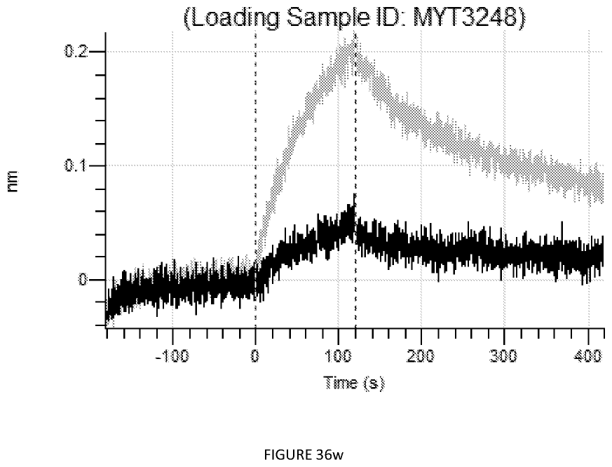

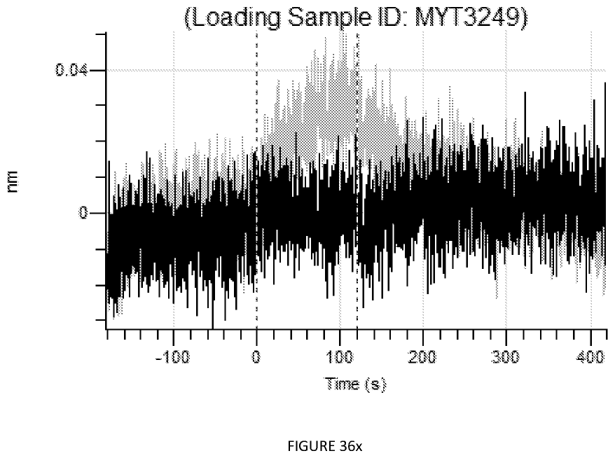

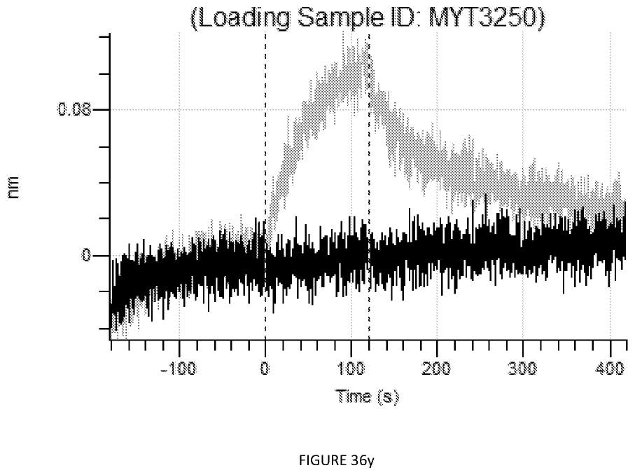

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.13 (U.S. Pat. No. 9,089,617 B2) (Heavy chain SEQ ID NO: 236, Light chain SEQ ID NO: 237) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3226-MYT3251). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 35), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 36, were selected for further analysis (e.g., MYT3227, MYT3231, MYT3234, MYT3235, MYT3236, MYT3237, MYT3245, MYT3246, MYT3248, and MYT3250). It was also noted that while some histidine and alanine mutations obliterated DLL3 binding (e.g., MYT3249), others were tolerated with little (e.g., less than 1-fold change in dissociation constant KD or dissociation rate) or no change in DLL3 binding kinetics (e.g., MYT3226, MYT3228, MYT3229, MYT3230, MYT3232, MYT3233, MYT3238, MYT3239, MYT3240, MYT3241, MYT3242, MYT3243, MYT3244, MYT3247, and MYT3251). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 21. Construction and Screening of pH-Engineered DLL3 ABPCs

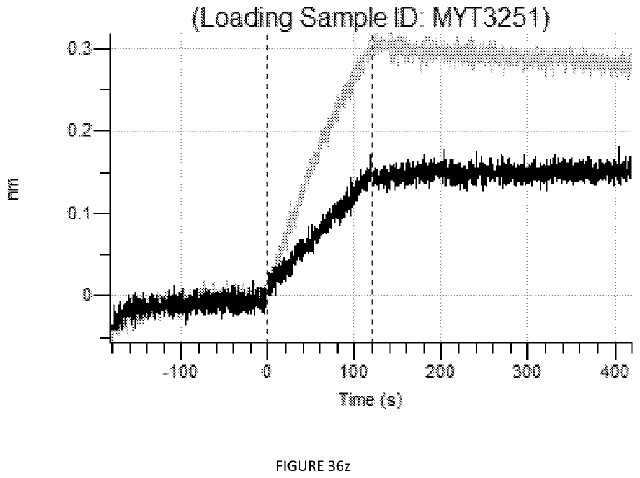

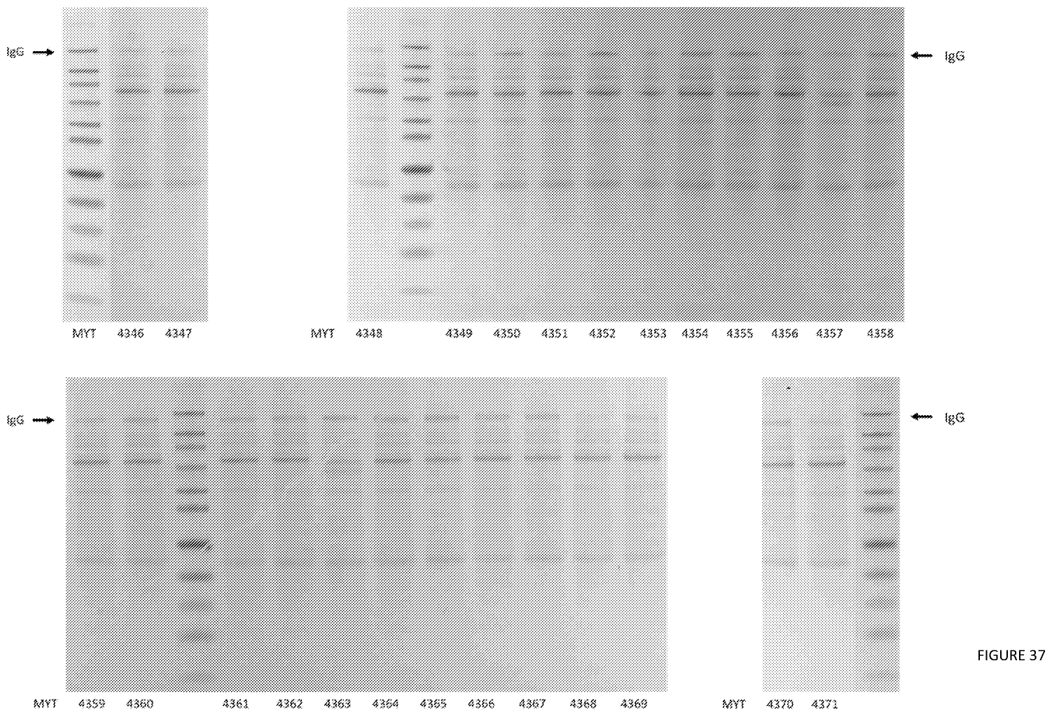

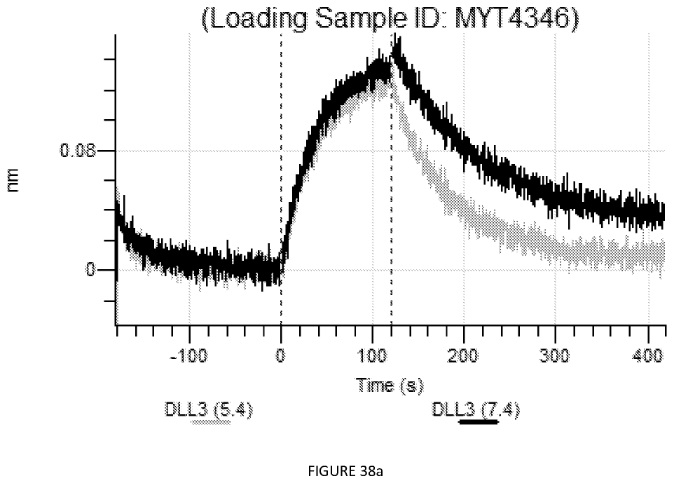

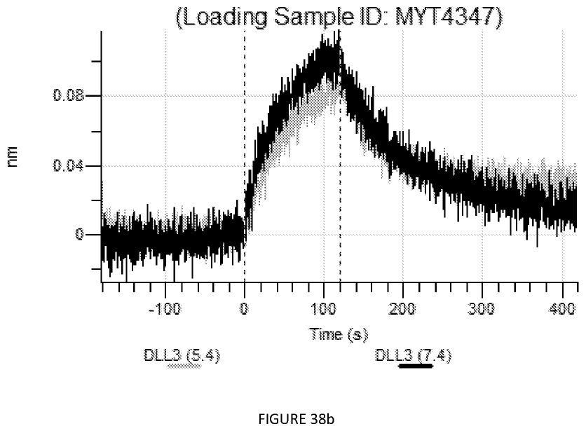

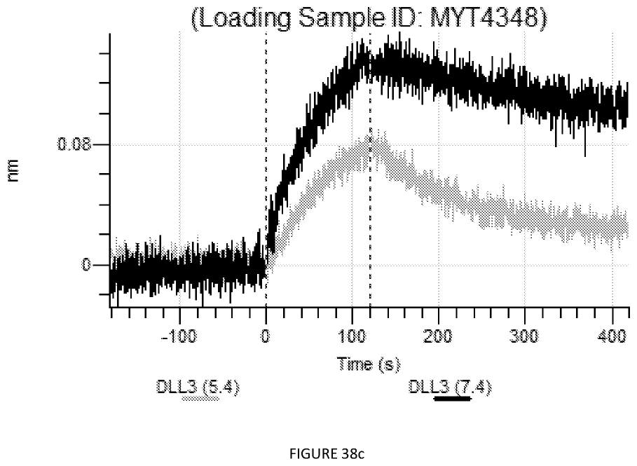

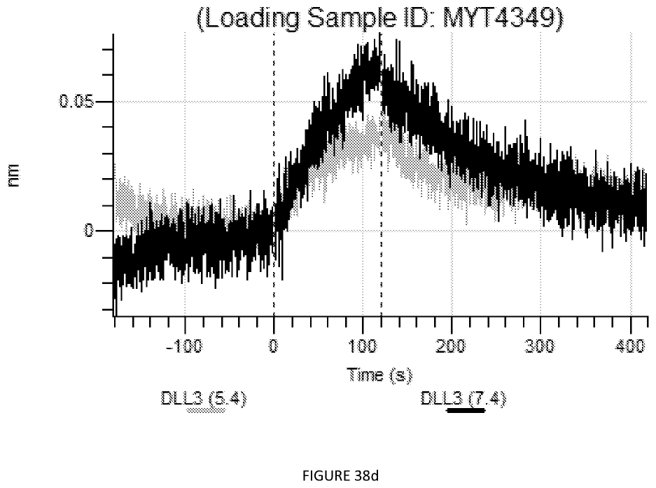

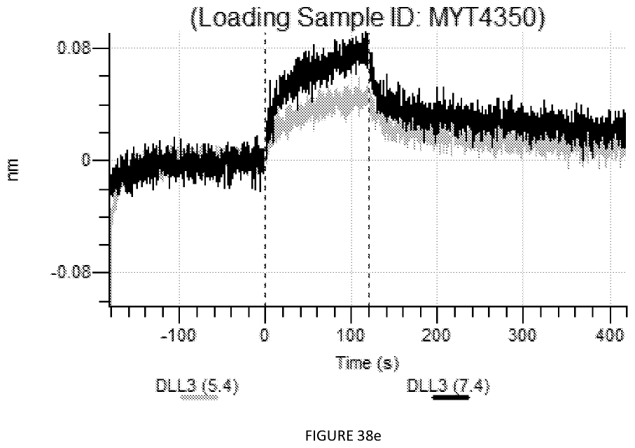

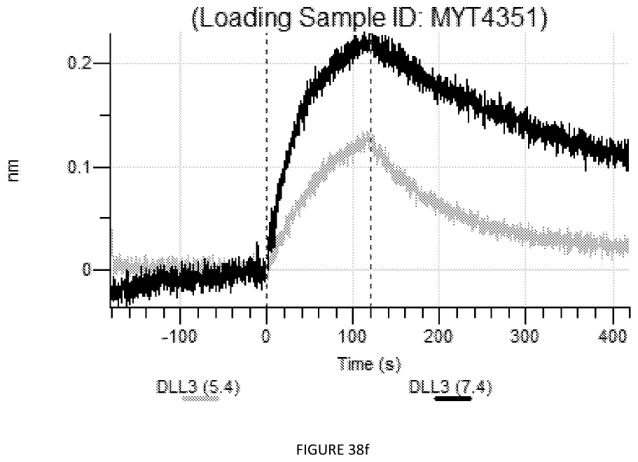

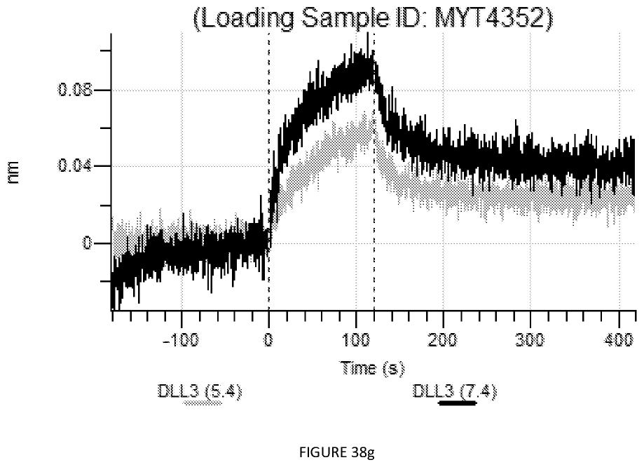

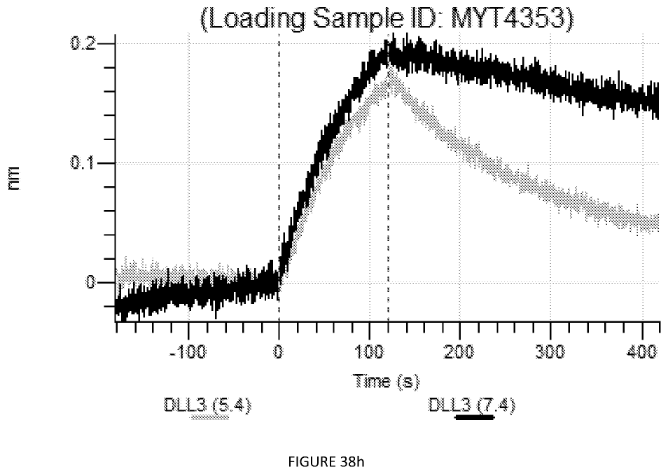

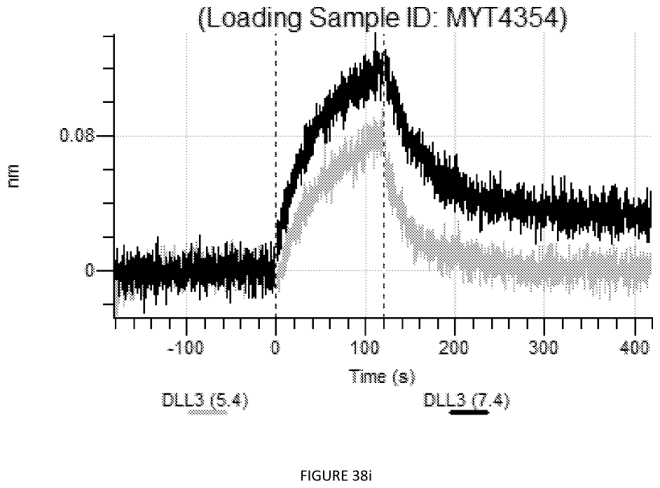

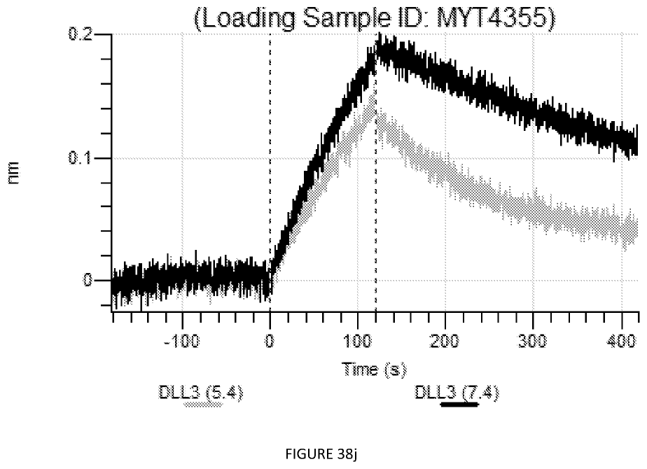

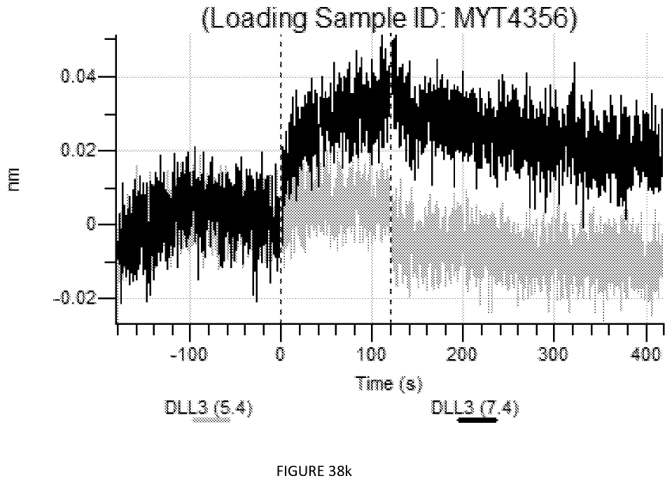

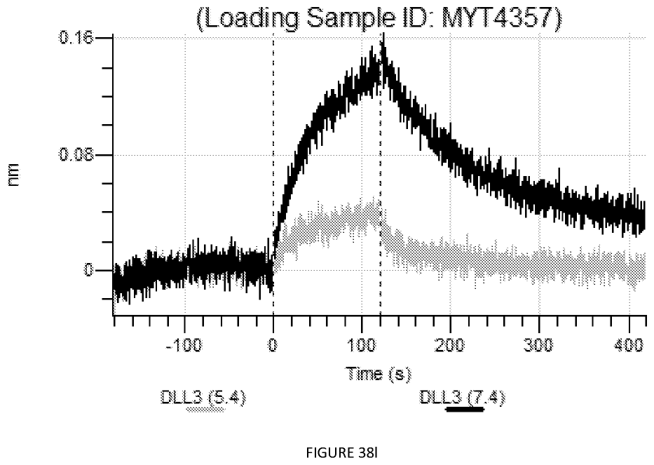

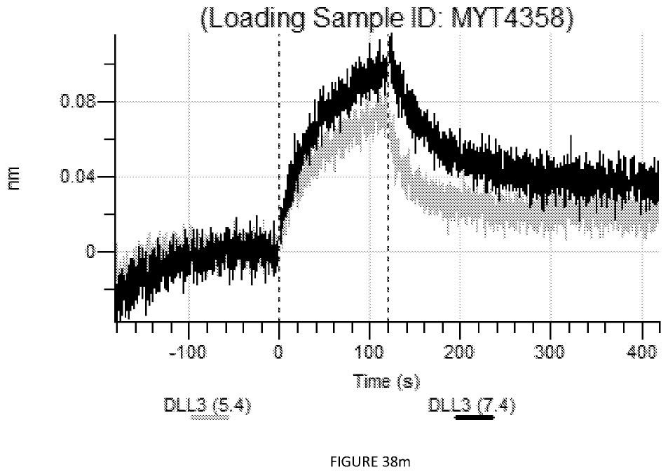

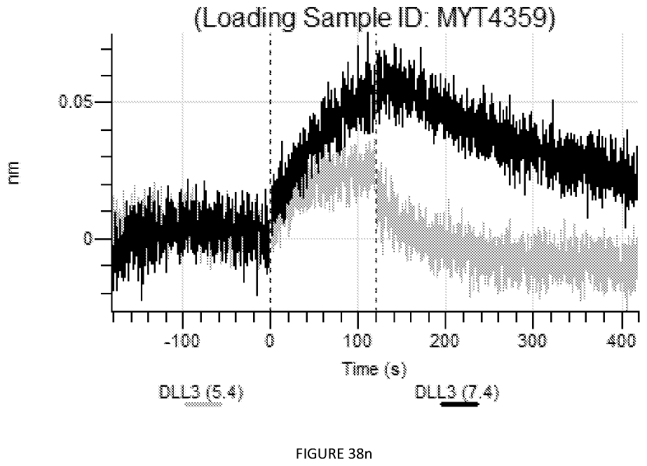

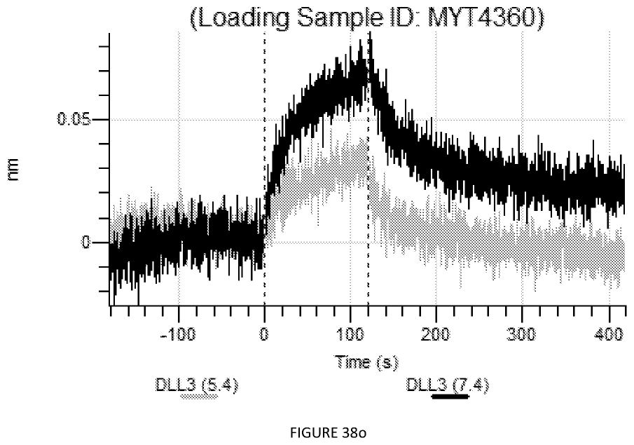

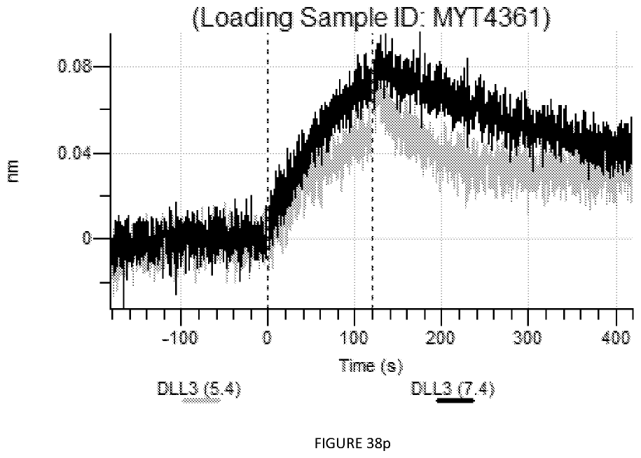

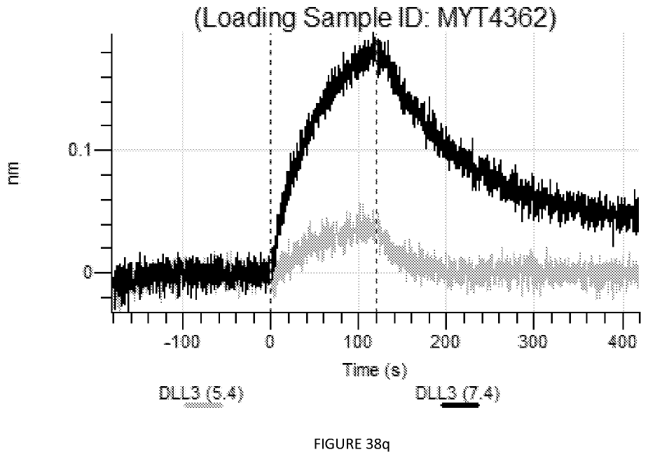

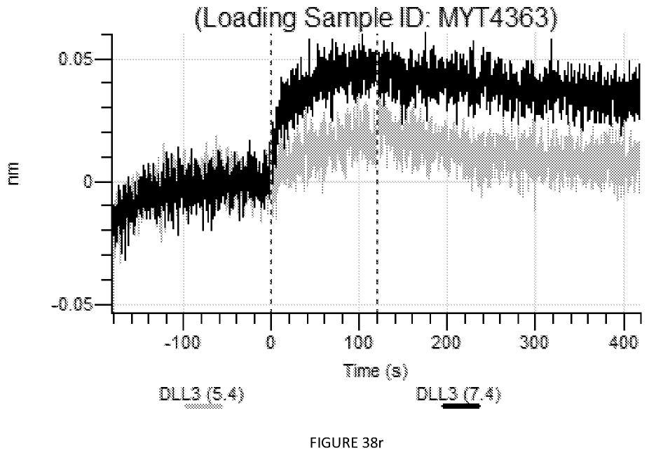

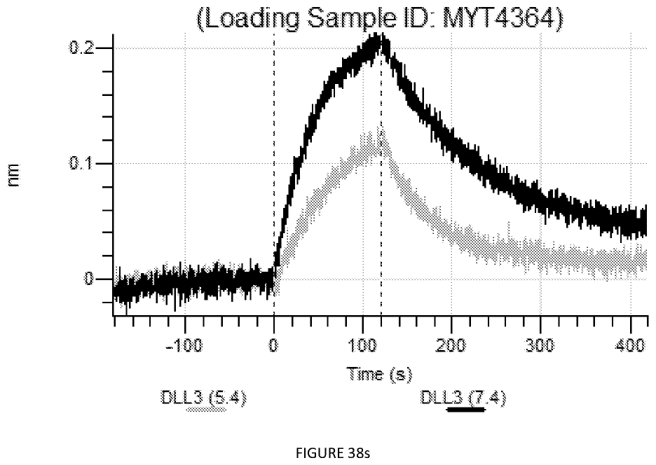

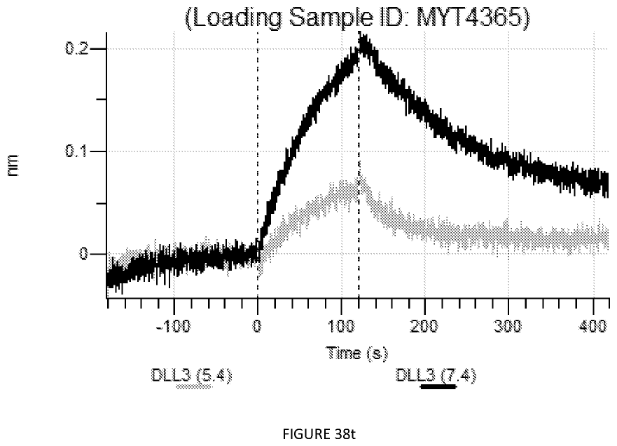

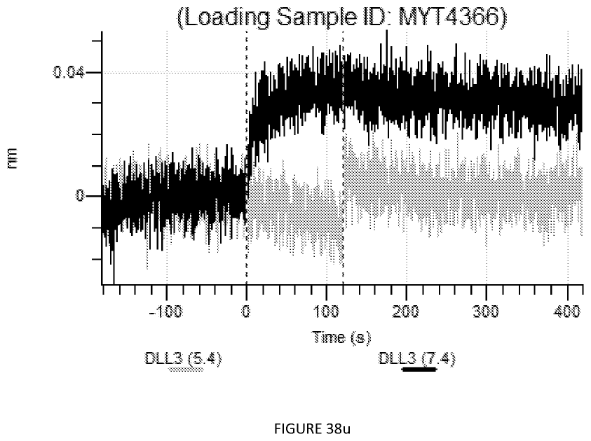

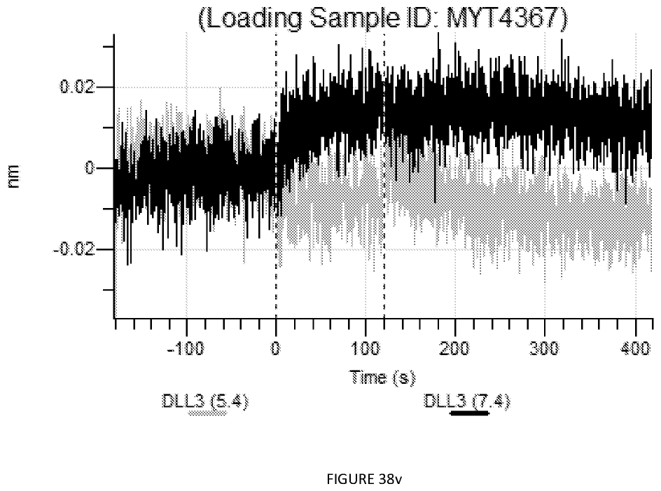

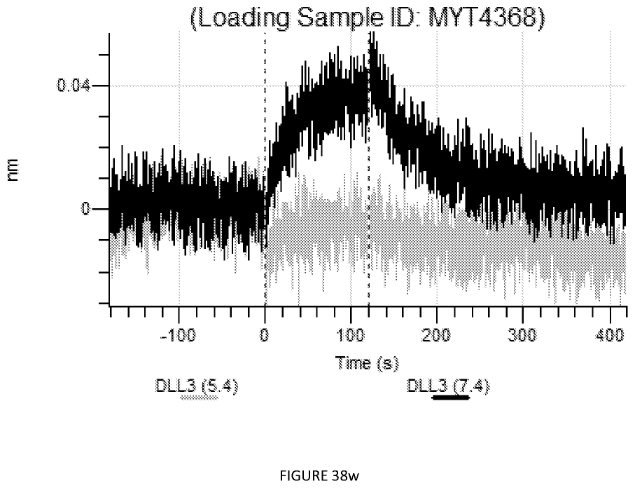

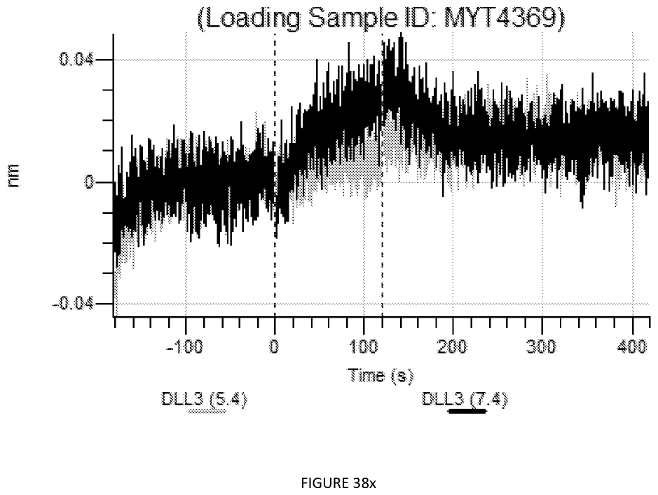

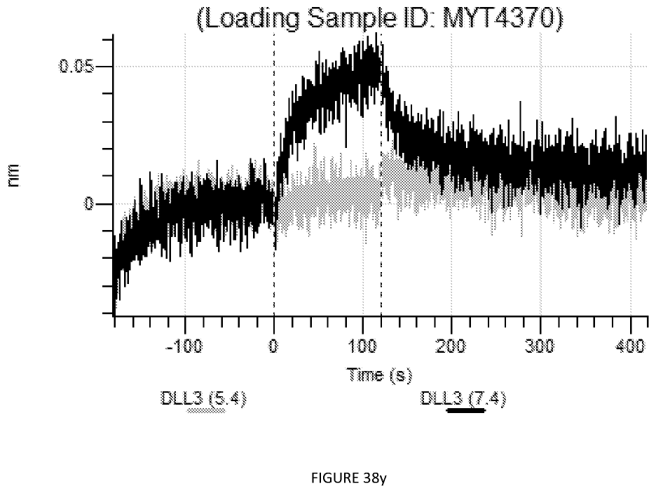

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.13 (U.S. Pat. No. 9,089,617 B2) (Heavy chain SEQ ID NO: 236, Light chain SEQ ID NO: 237) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy chain CDRs that had been previously selected for further analysis in Example 19 were systematically combined two or more at a time (MYT4346-MYT4371). In cases where the starting CDR residue was a histidine, it was mutated to an alanine. Antibody variants with two or more histidine or alanine mutations in the heavy chain CDRs were generated by co-transfection of Expi293 cells with a) one heavy chain combinations sequence variant, and b) the corresponding starting ABPC light chain using methods known to the art. After allowing for four days of protein expression, cell culture supernatants were collected, quantified by SDS-PAGE analysis (FIG. 37), and the pH dependence of the variant was evaluated using biolayer interferometry (BLI) on an Octet RED 96einstrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain combinations variants that showed either enhanced dissociation at pH 5.4 or reduced dissociation at pH 7.4 or both (as compared to the starting ABPC), as shown in FIG. 38 were selected for further analysis (e.g, MYT4346-MYT4366, MYT4368, MYT4369, and MYT4370).

### Example 22. Construction and Screening of pH-Engineered DLL3 ABPCs

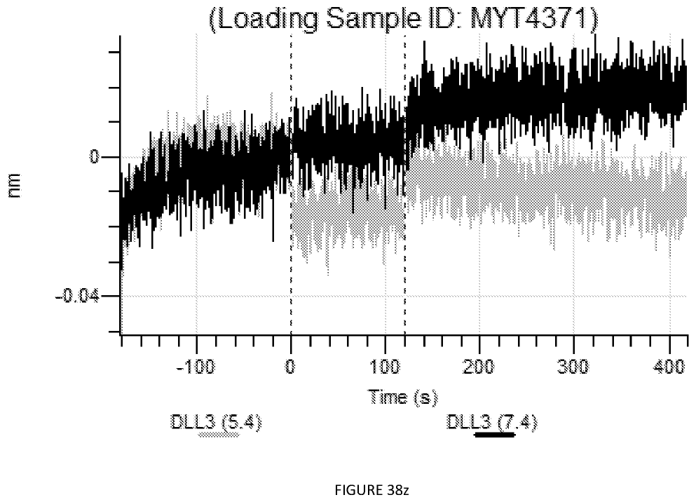

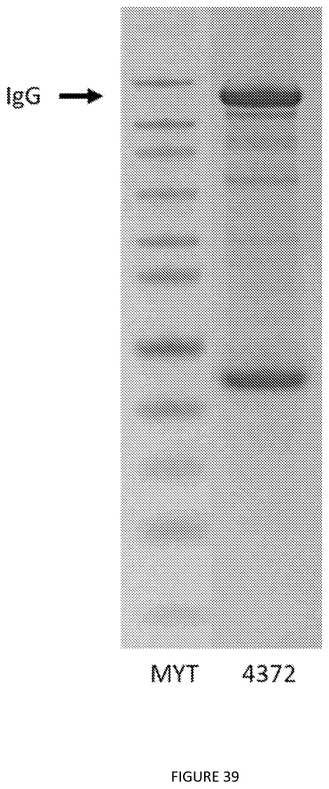

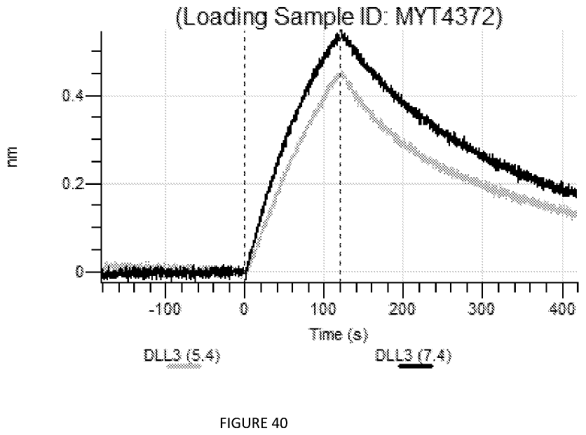

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.13 (U.S. Pat. No. 9,089,617 B2) (Heavy chain SEQ ID NO: 236, Light chain SEQ ID NO: 237) were selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the light chain CDRs that had been previously selected for further analysis in Example 20 were systematically combined two or more at a time (MYT4372). In cases where the starting CDR residue was a histidine, it was mutated to an alanine. Antibody variants with two or more histidine or alanine mutations in the light chain CDRs were generated by co-transfection of Expi293 cells with a) one light chain combinations sequence variant, and b) the corresponding starting ABPC heavy chain using methods known to the art. After allowing for four days of protein expression, cell culture supernatants were collected, quantified by SDS-PAGE analysis (FIG. 39), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain combinations variants that showed either enhanced dissociation at pH 5.4 or reduced dissociation at pH 7.4 or both (as compared to the starting ABPC), as shown in FIG. 40, were selected for further analysis (MYT4372).

### Example 23. Construction and Screening of pH-Engineered DLL3 ABPCs

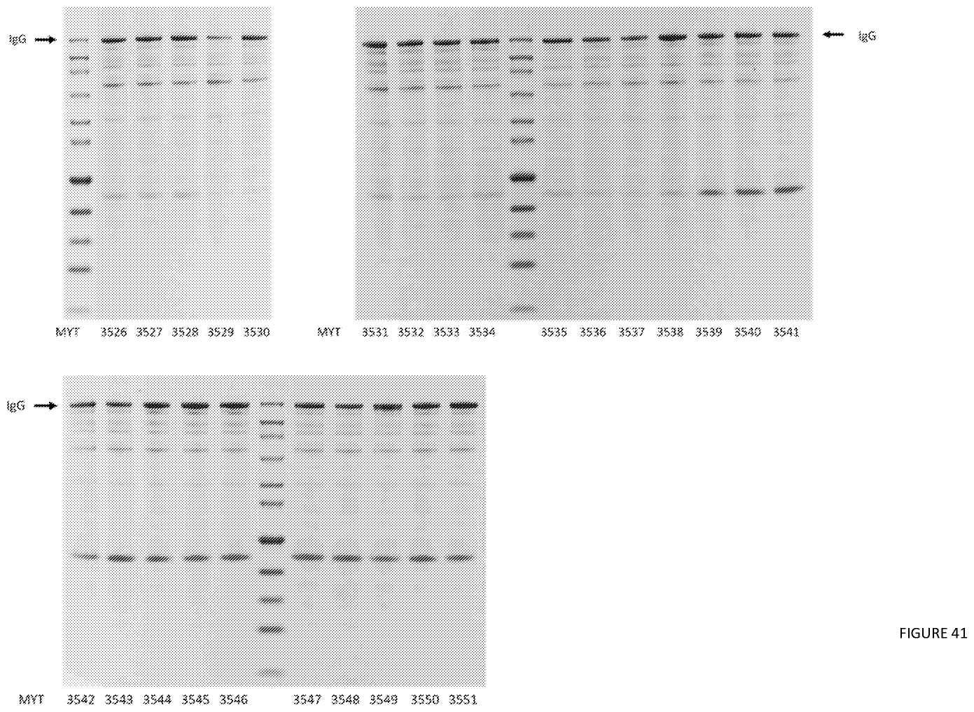

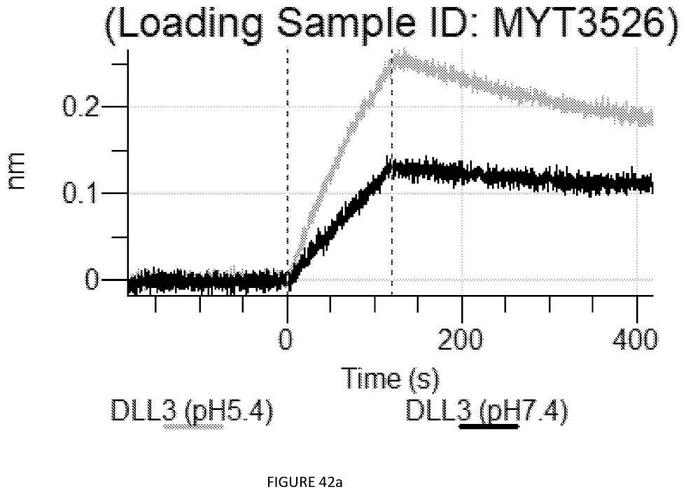

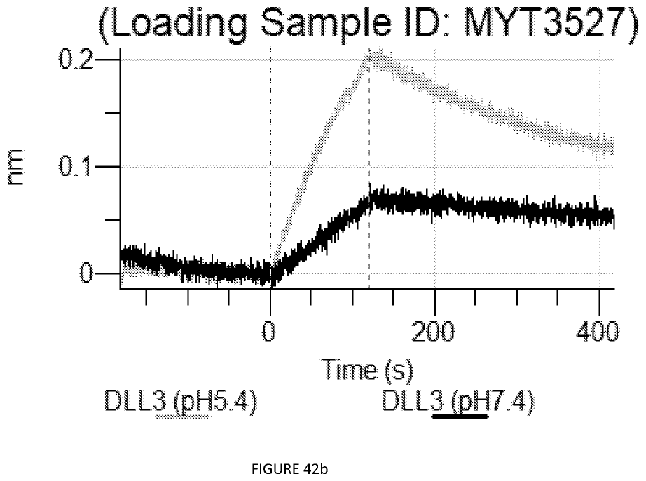

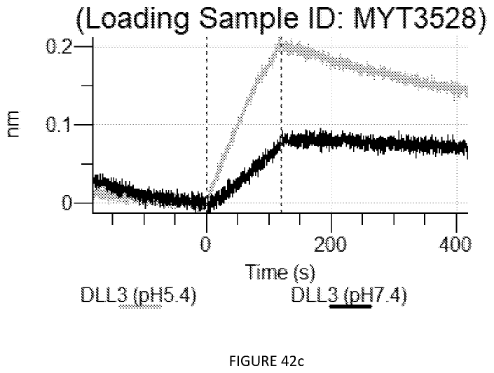

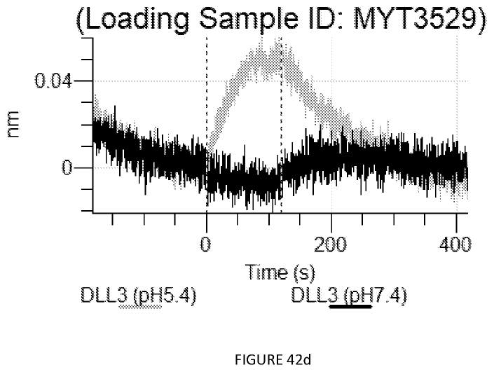

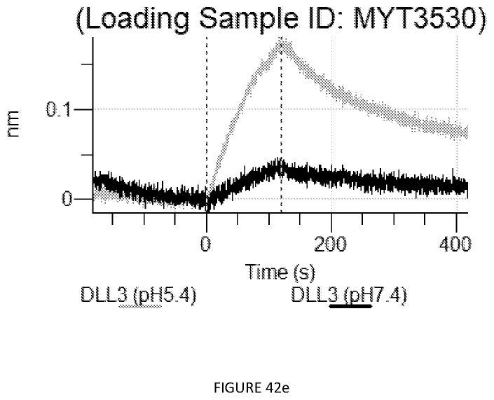

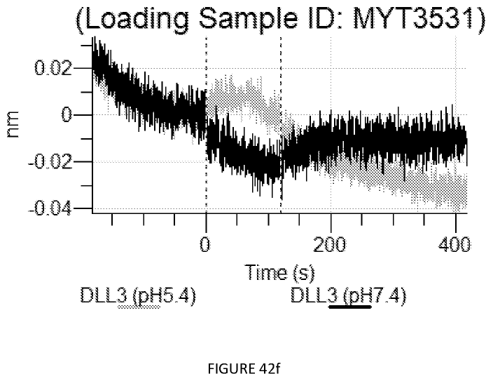

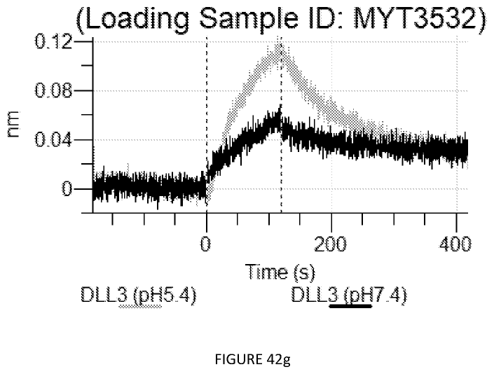

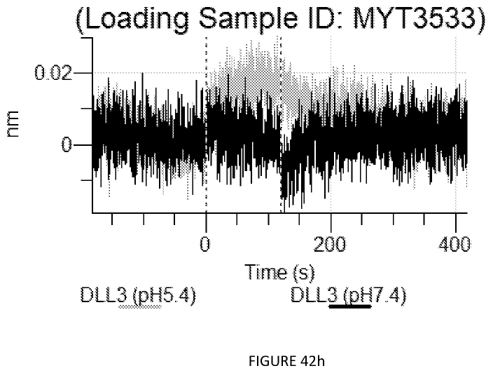

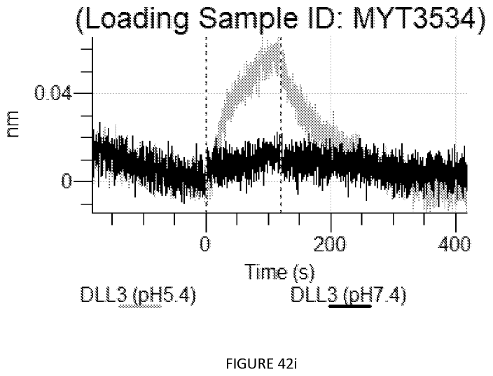

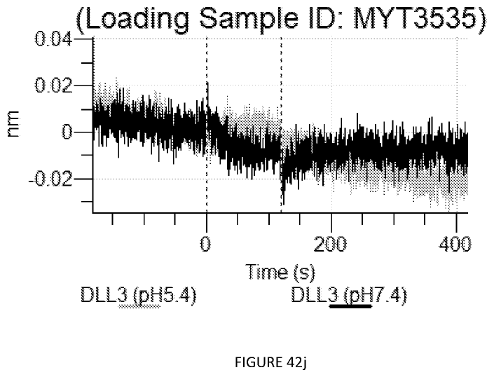

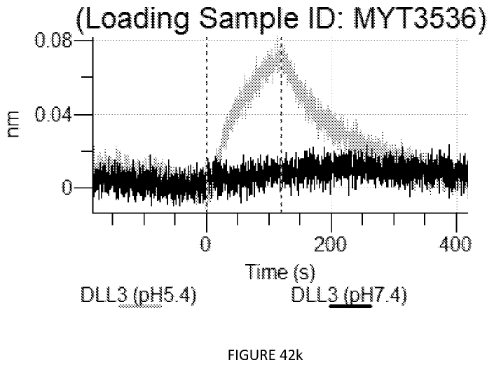

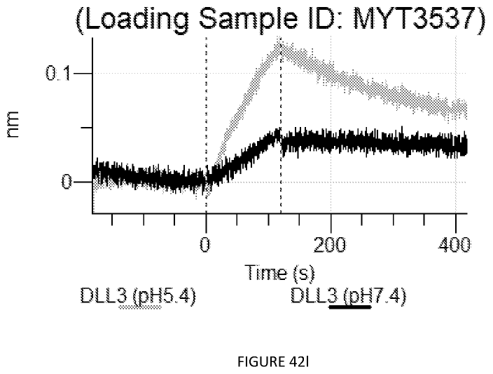

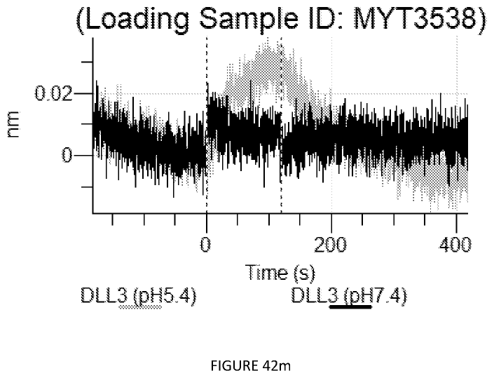

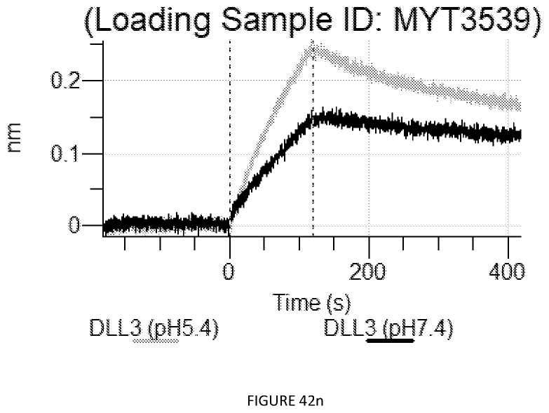

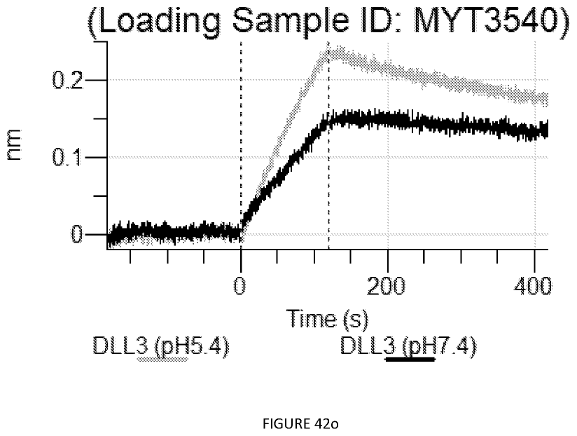

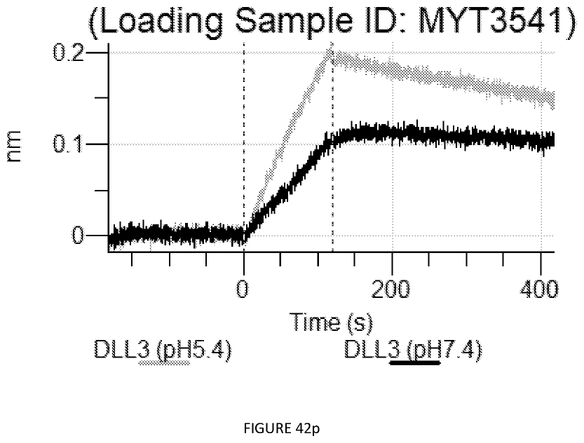

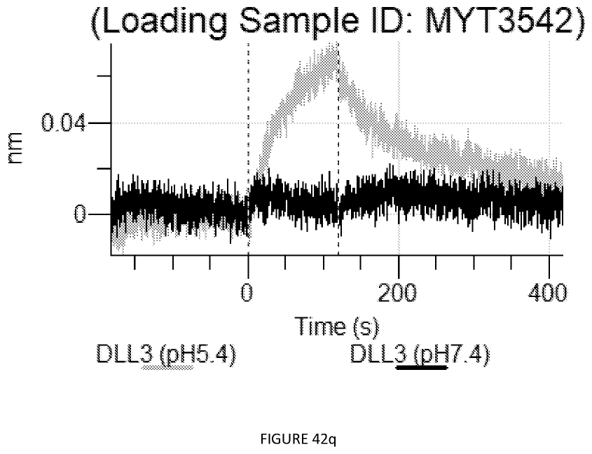

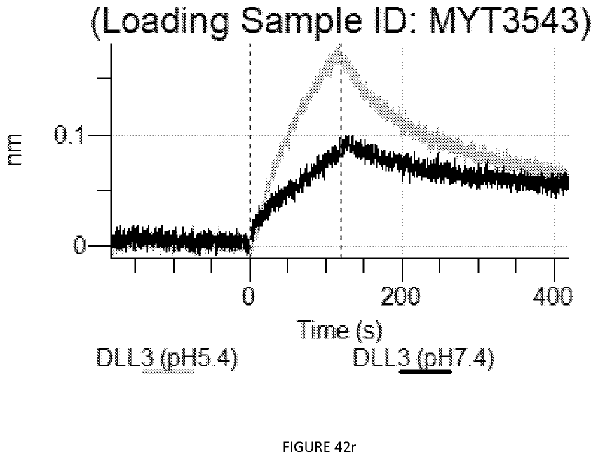

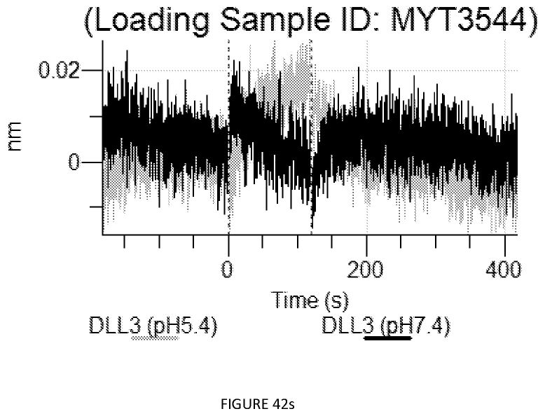

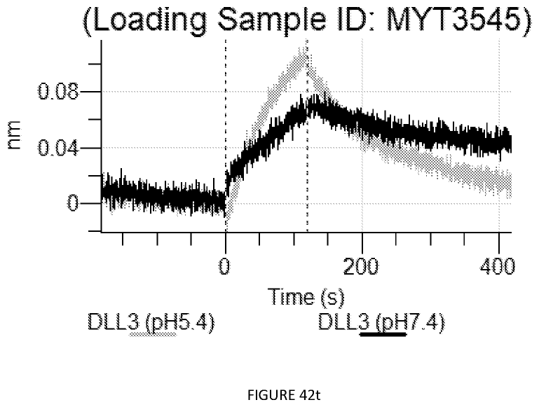

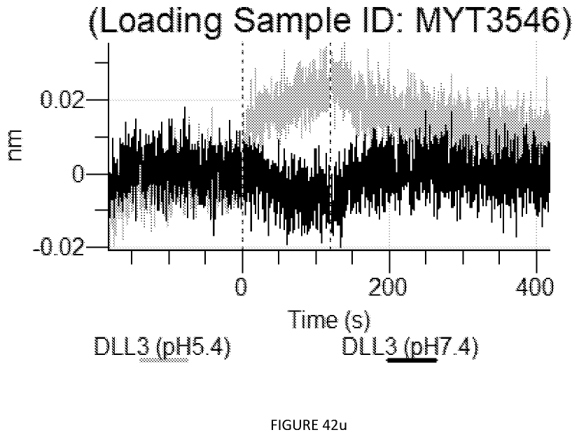

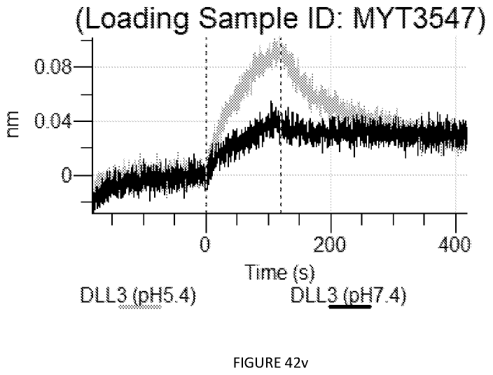

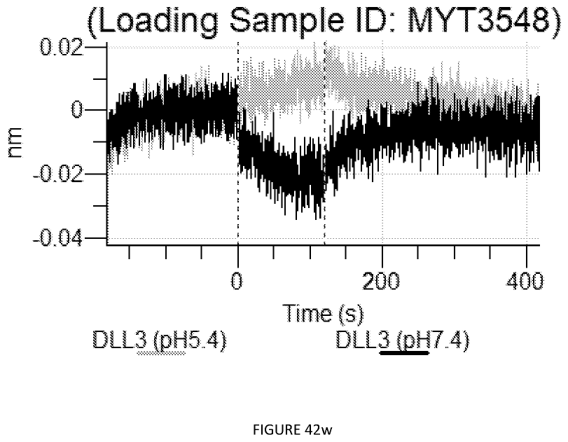

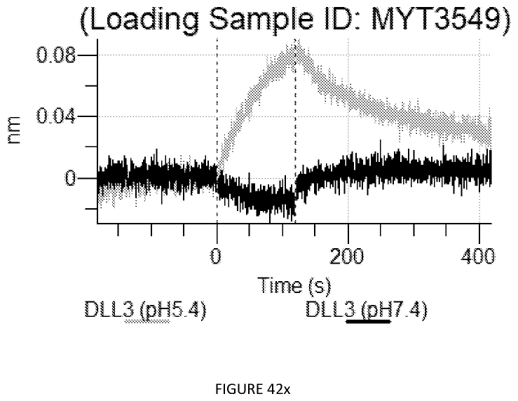

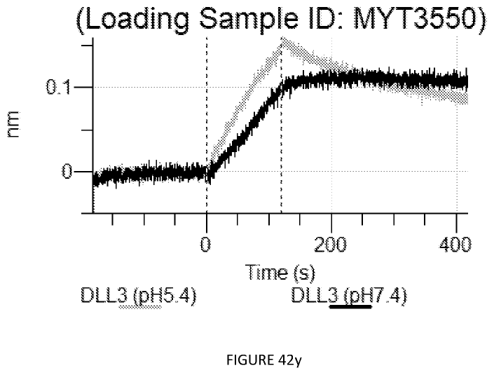

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.13 (U.S. Pat. No. 9,089,617 B2) (Heavy chain SEQ ID NO: 236, Light chain SEQ ID NO: 237) was selected as a DLL3-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy and light chain CDRs that had been previously selected for further analysis in Examples 19-22 were systematically combined two or more at a time (MYT3526-3551). In cases where the starting CDR residue was a histidine, it was mutated to an alanine. Antibody variants with two or more histidine or alanine mutations were generated by co-transfection of Expi293 cells with a) one light chain sequence variant, and b) one heavy chain sequence variant using methods known to the art. After allowing for four days of protein expression, cell culture supernatants were collected, quantified by SDS-PAGE analysis (FIG. 41), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Paired heavy and light chain variants that showed either enhanced dissociation at pH 5.4 or reduced dissociation at pH 7.4 or both (as compared to the starting ABPC), as shown in FIG. 42, were selected for further analysis (MYT3526-MYT3530, MYT3532, MYT3534, MYT3536-MYT3537, MYT3539-MYT3543, MYT3545, MYT3547, and MYT3549-3551).

### Example 24. Construction and Screening of pH-Engineered DLL3 ABPCs

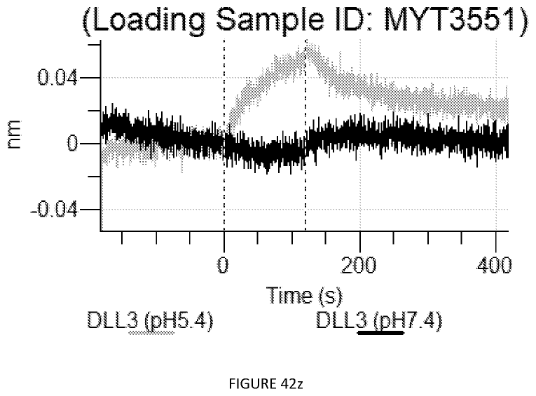

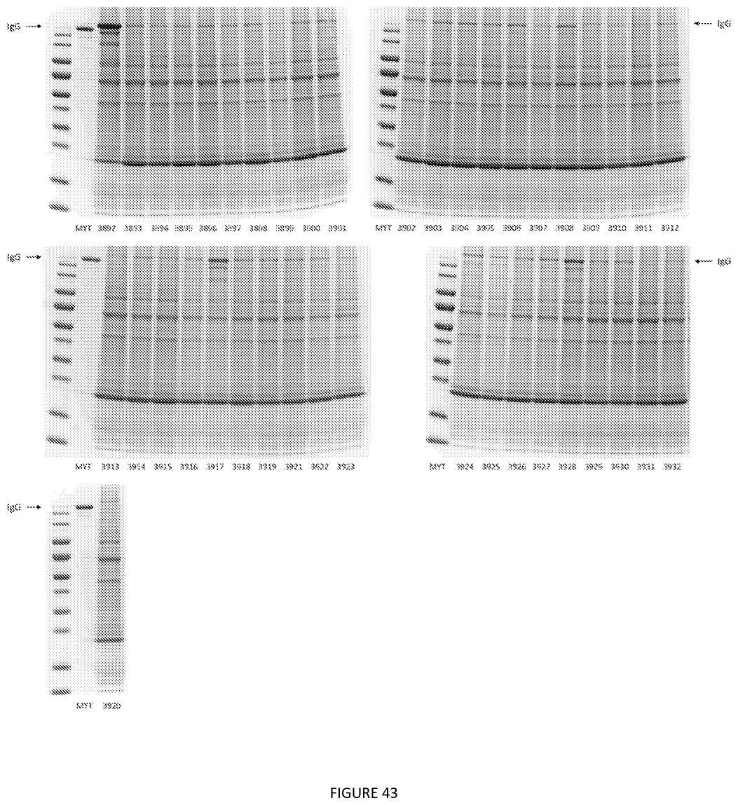

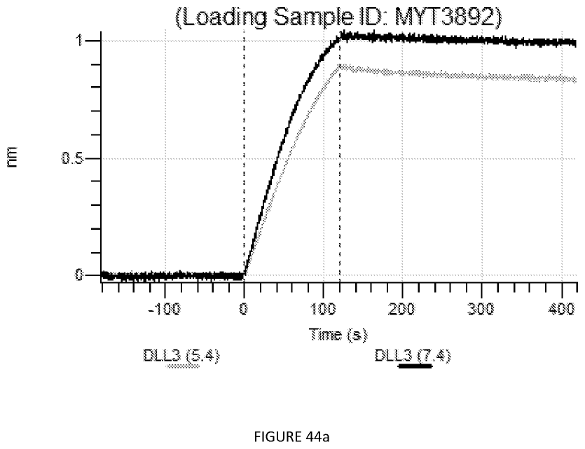

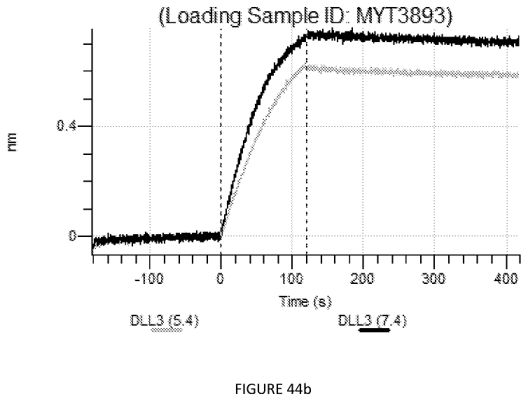

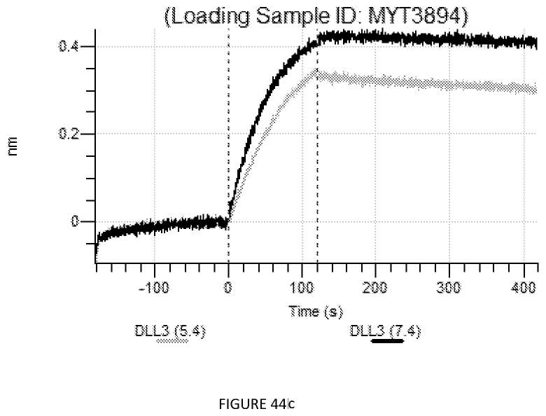

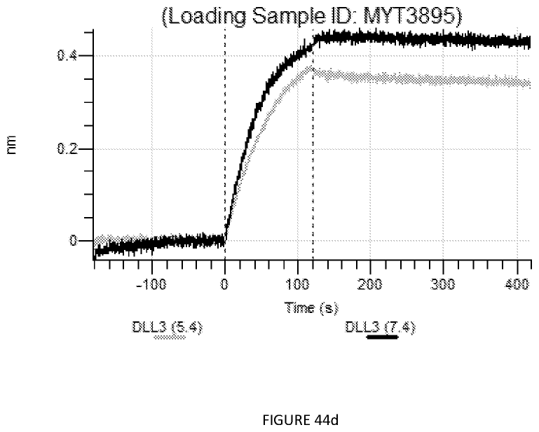

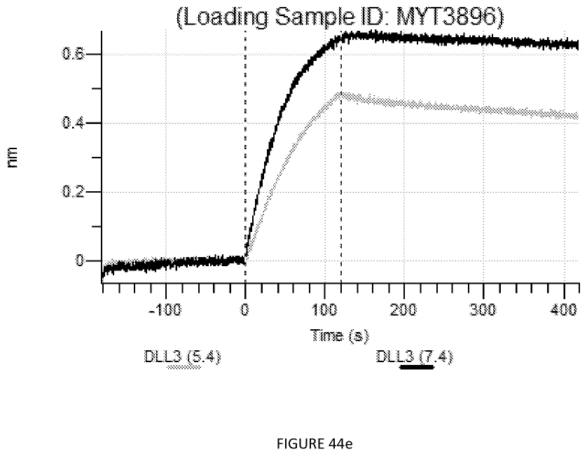

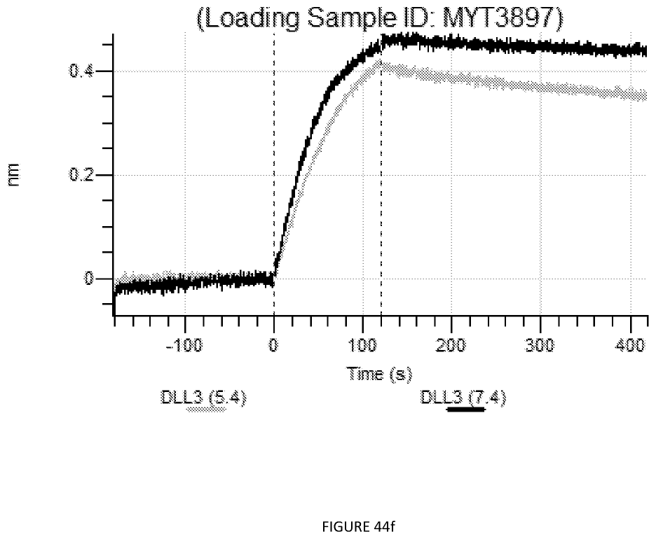

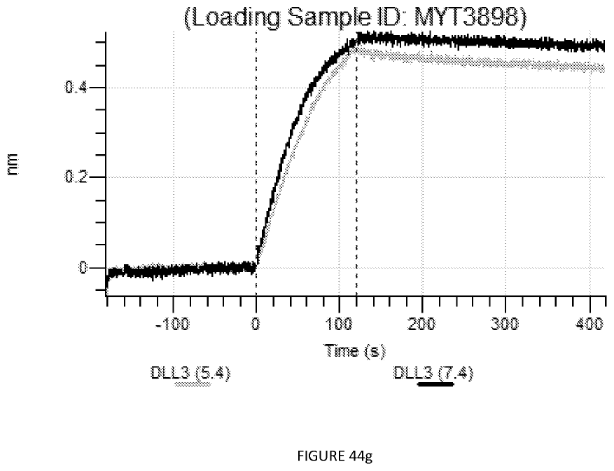

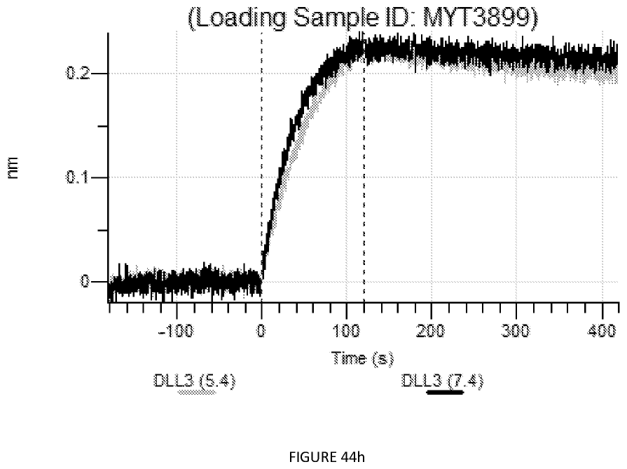

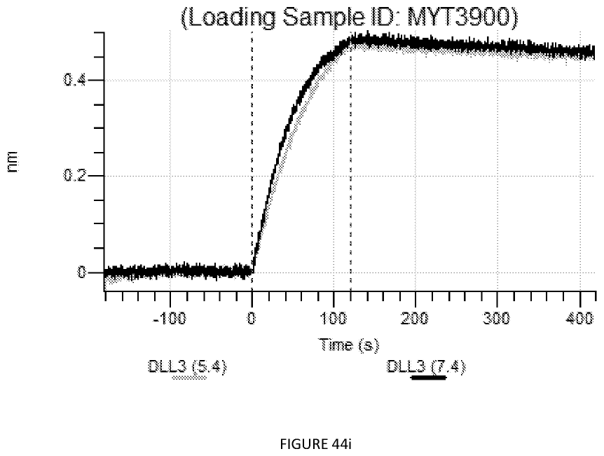

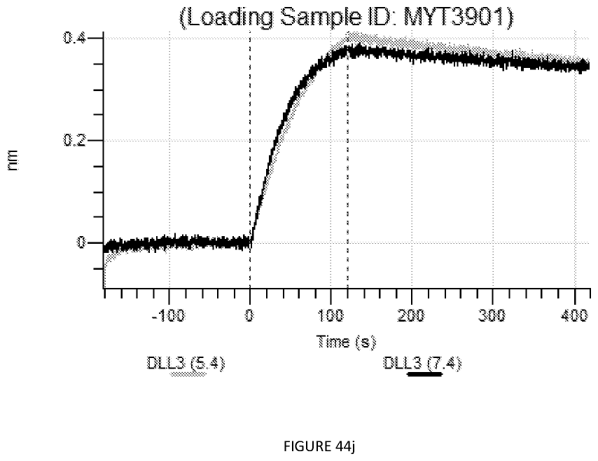

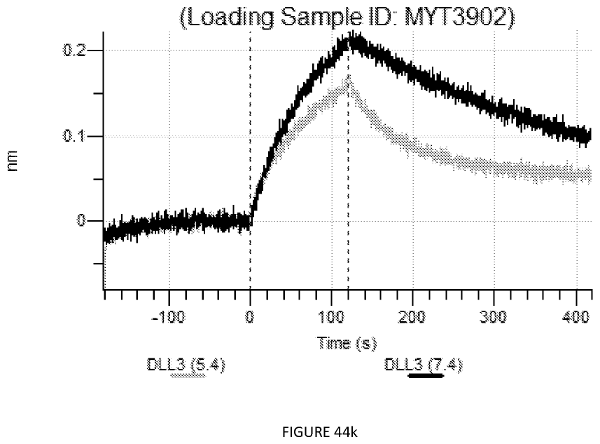

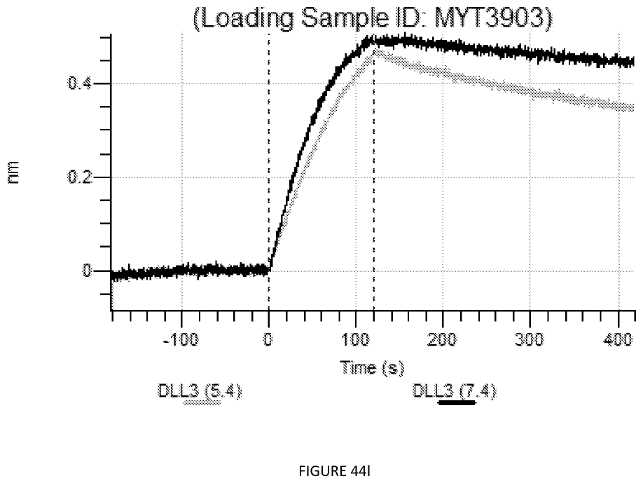

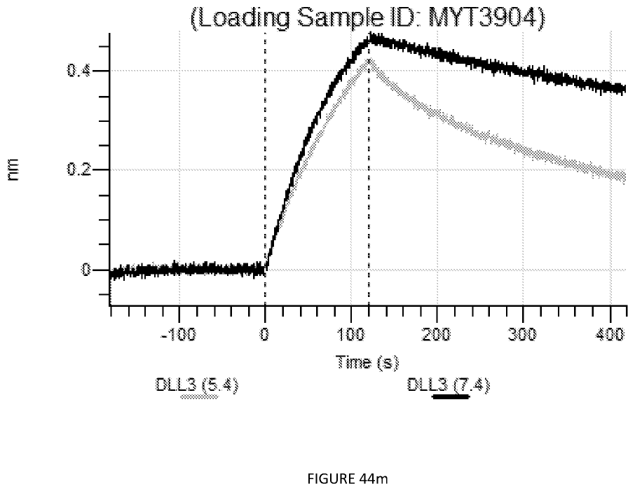

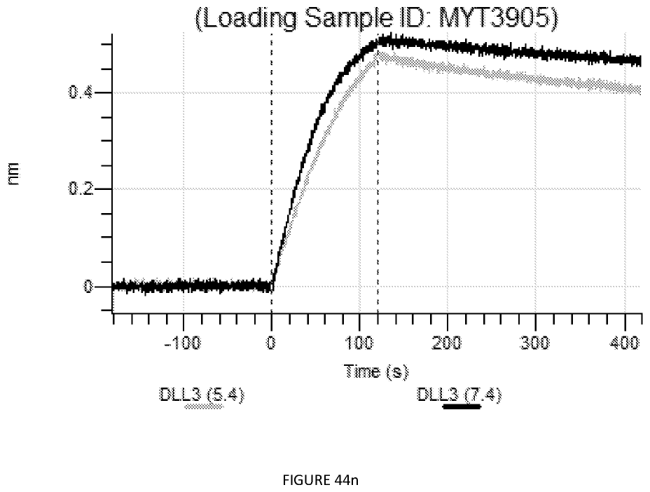

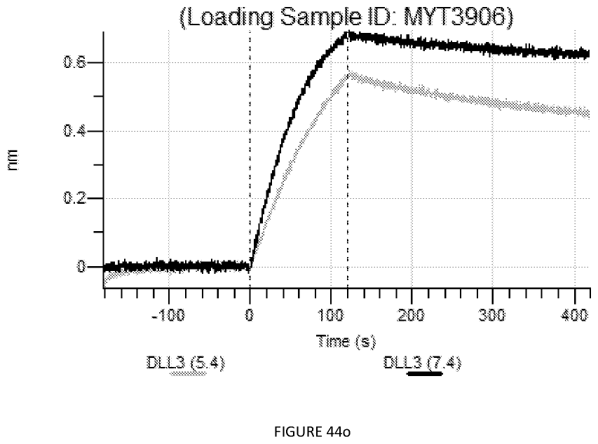

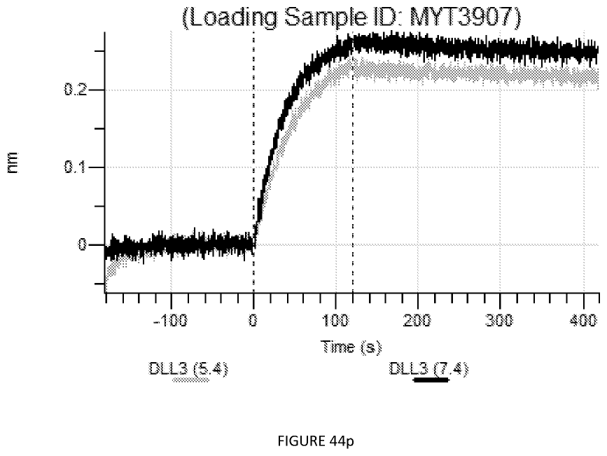

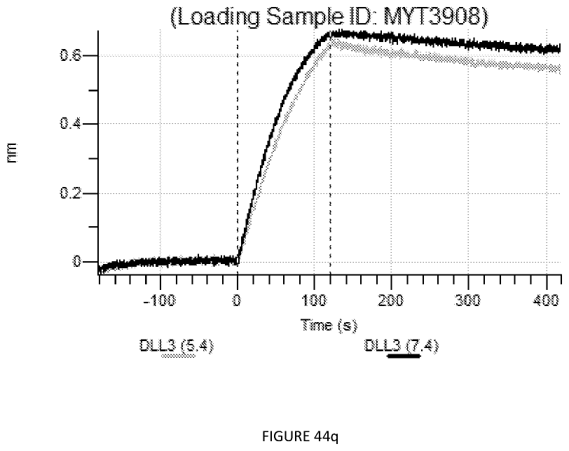

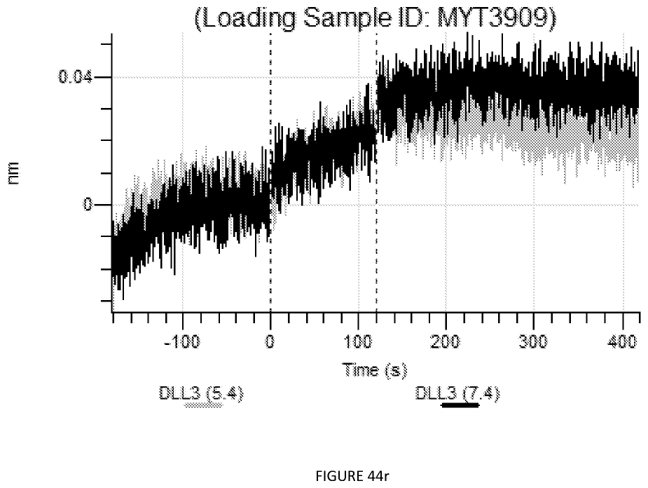

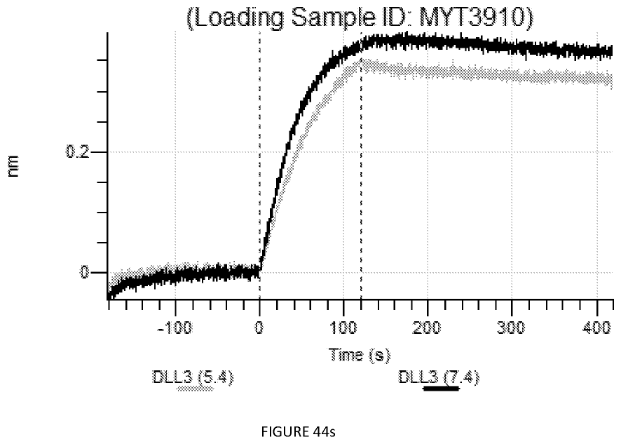

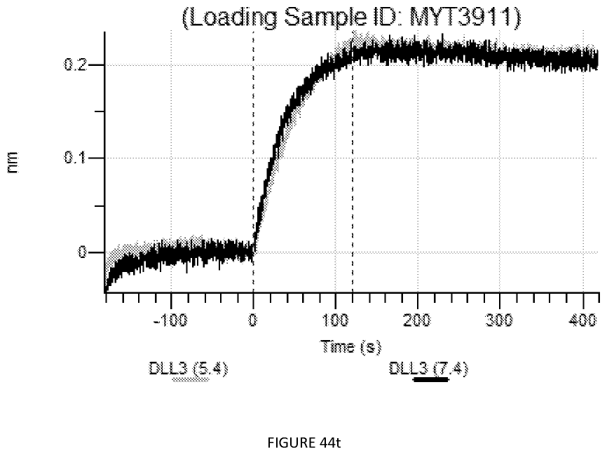

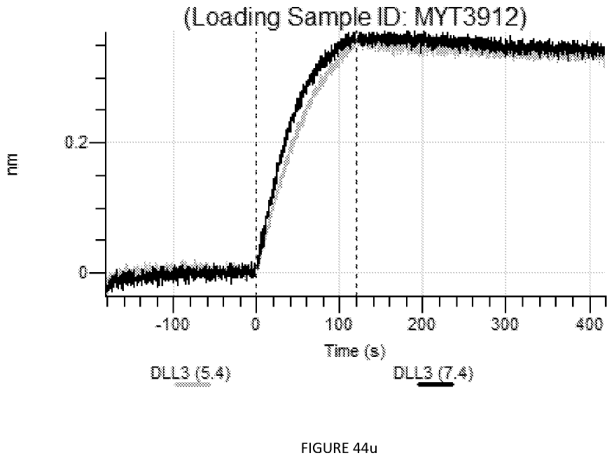

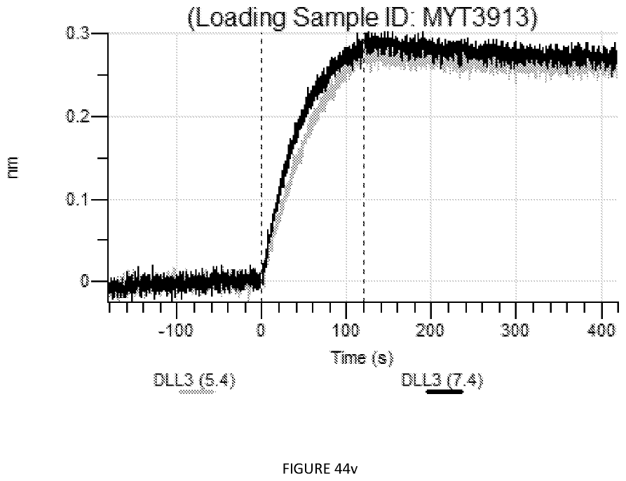

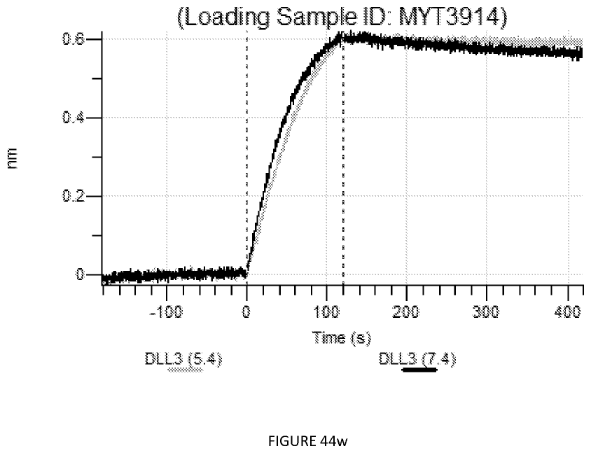

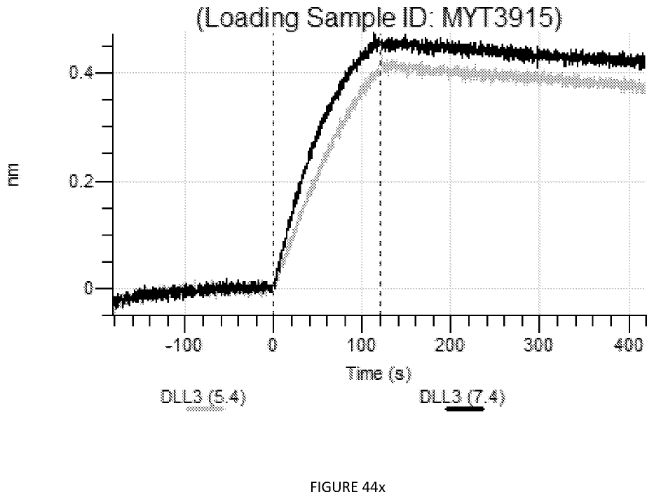

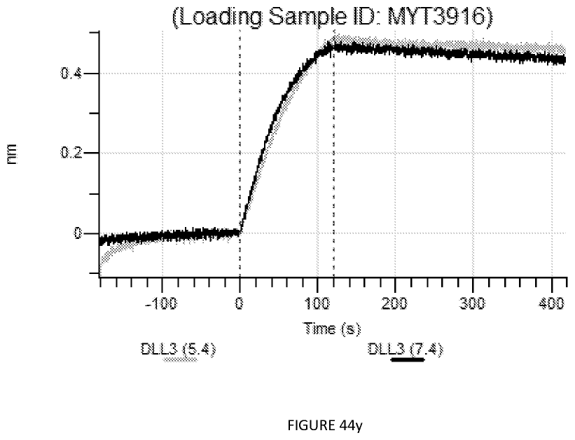

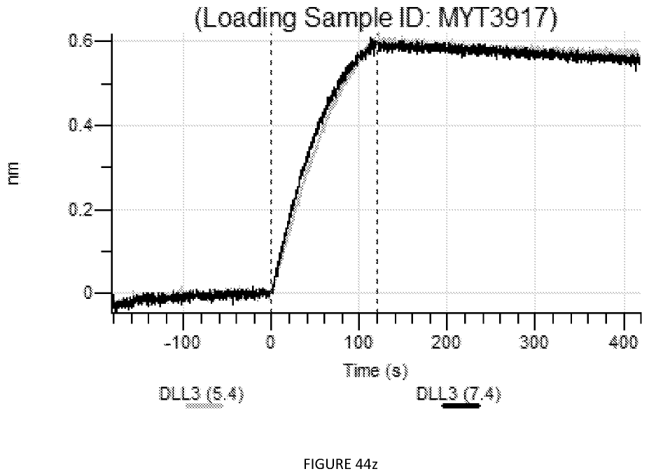

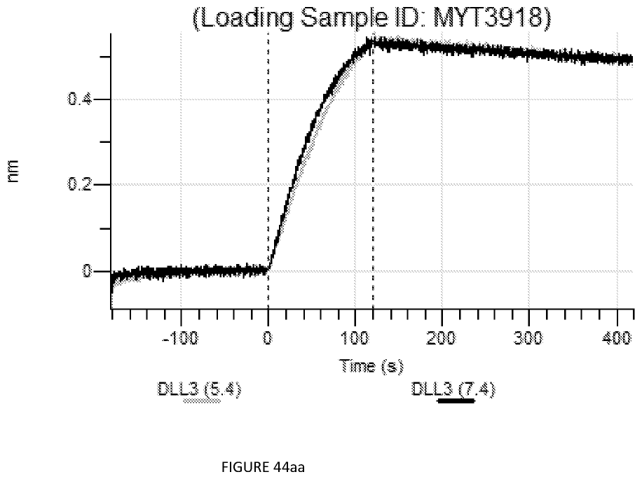

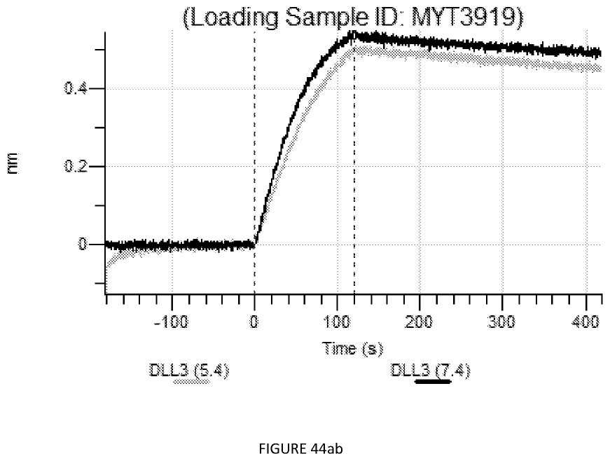

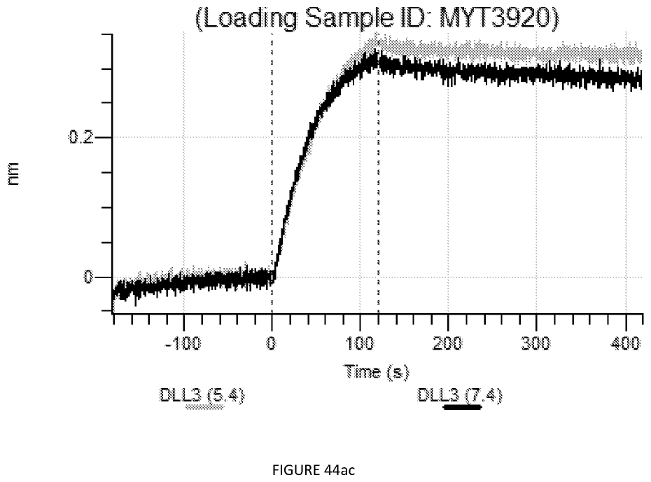

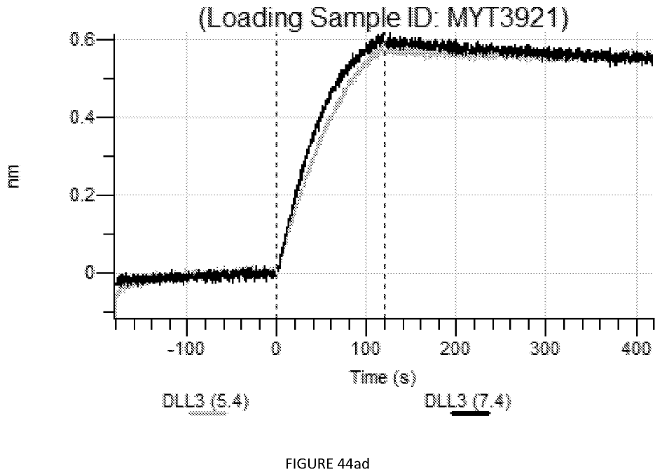

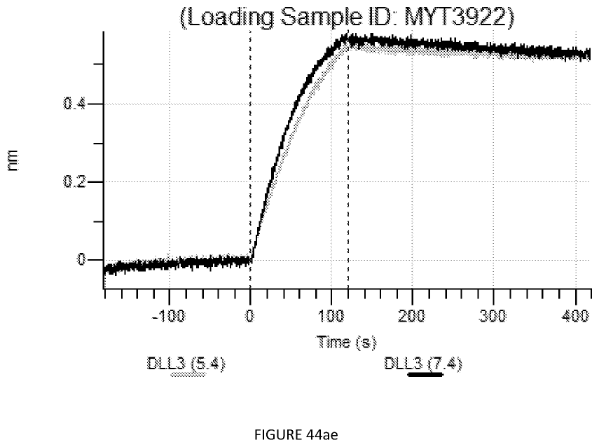

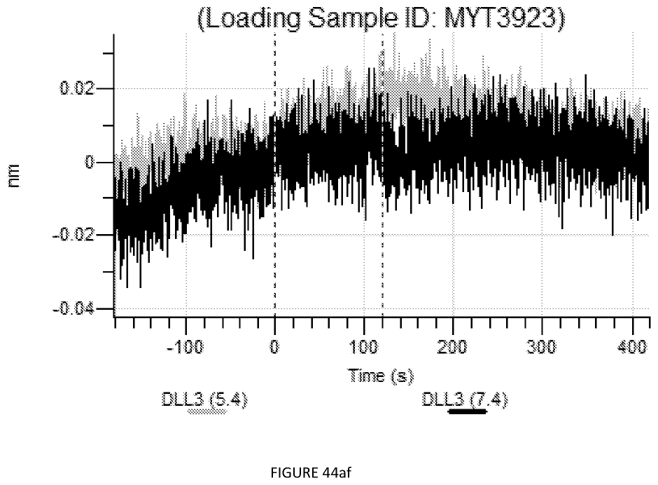

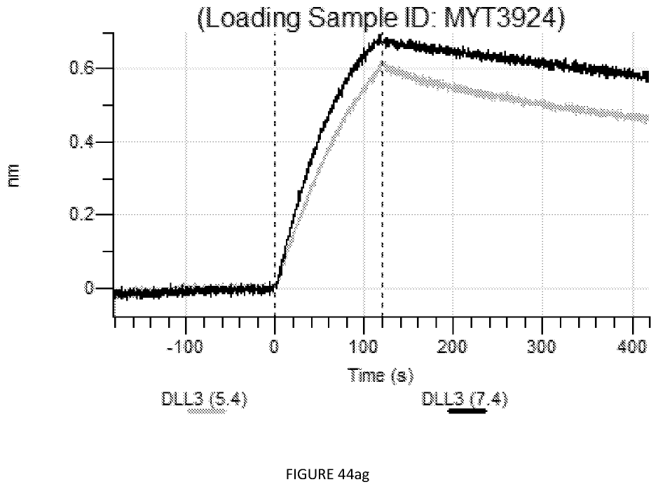

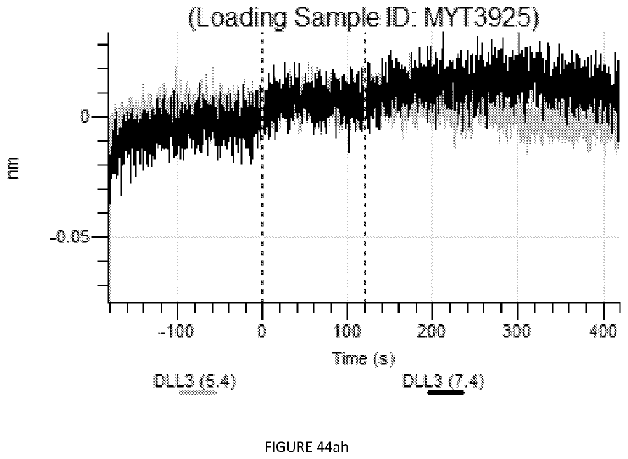

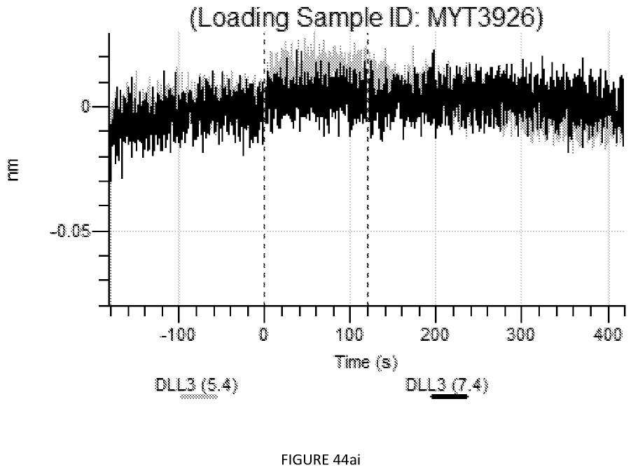

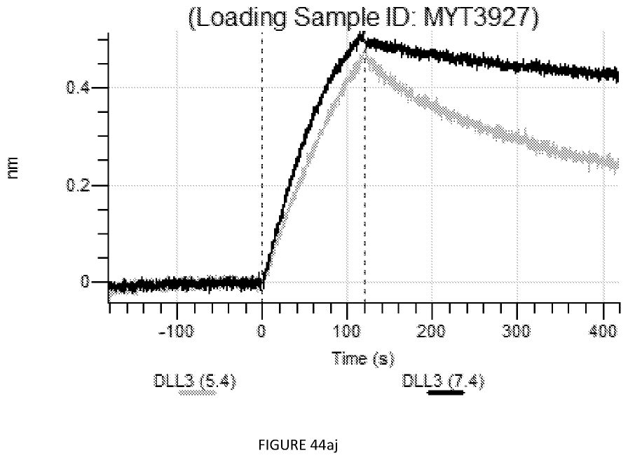

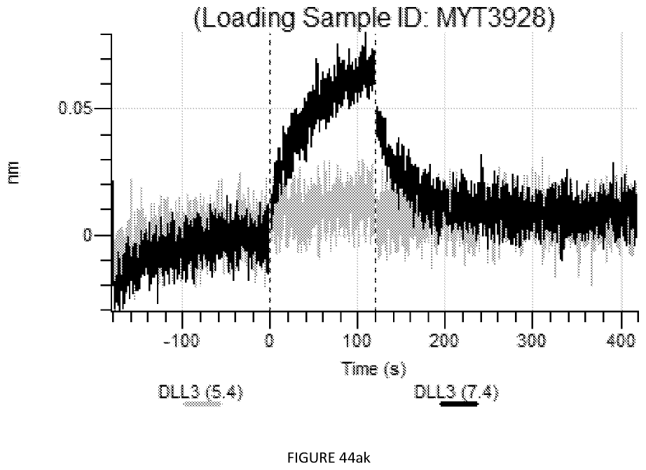

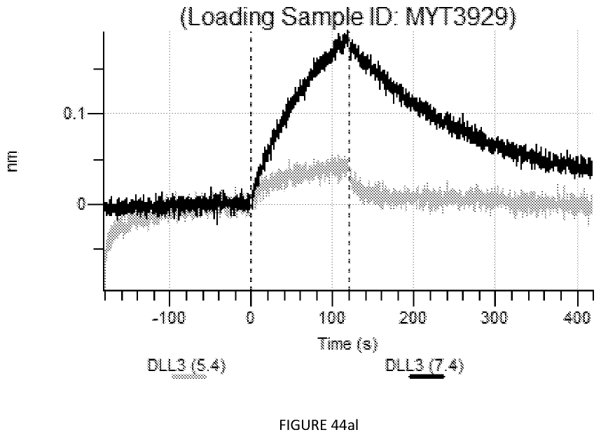

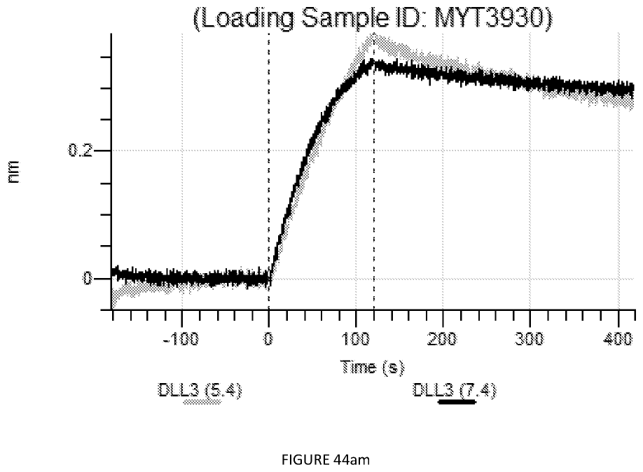

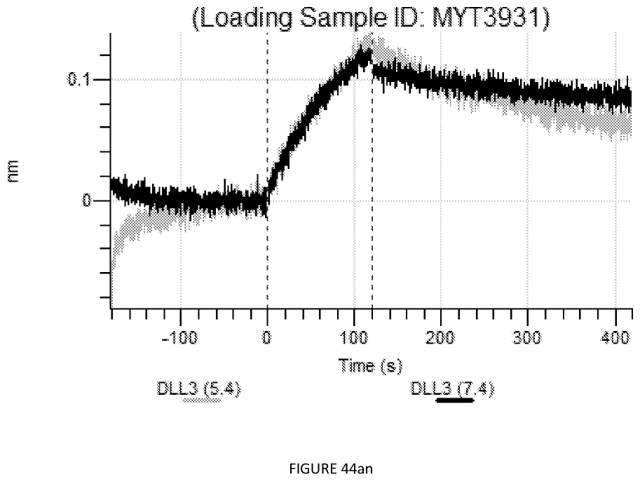

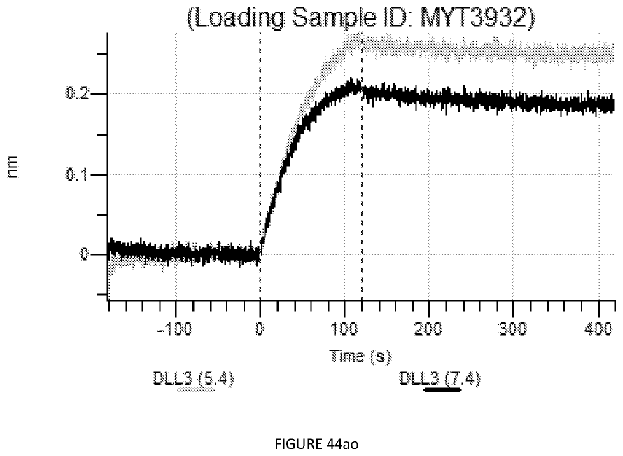

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.15 (U.S. Pat. No. 9,089,617 B2) (Heavy chain SEQ ID NO: 335, Light chain SEQ ID NO: 336) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3893-3932). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 43) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 44 were selected for further analysis (MYT3897, MYT3902-MYT3906, MYT3924, and MYT3927-MYT3931). It was also noted that while some histidine and alanine mutations obliterated DLL3 binding (e.g., MYT3909, MYT3923, MYT3925, and MYT3926), others were tolerated with little (e.g., less than 1-fold change in dissociation constant KD or dissociation rate) or no change in DLL3 binding kinetics (e.g., MYT3893, MYT3894, MYT3895, MYT3896, MYT3898, MYT3899, MYT3900, MYT3901, MYT3907, MYT3908, MYT3910, MYT3911, MYT3912, MYT3913, MYT3914, MYT3915, MYT3916, MYT3917, MYT3918, MYT3919, MYT3920, MYT3921, MYT3922, and MYT3932). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 25. Construction and Screening of pH-Engineered DLL3 ABPCs

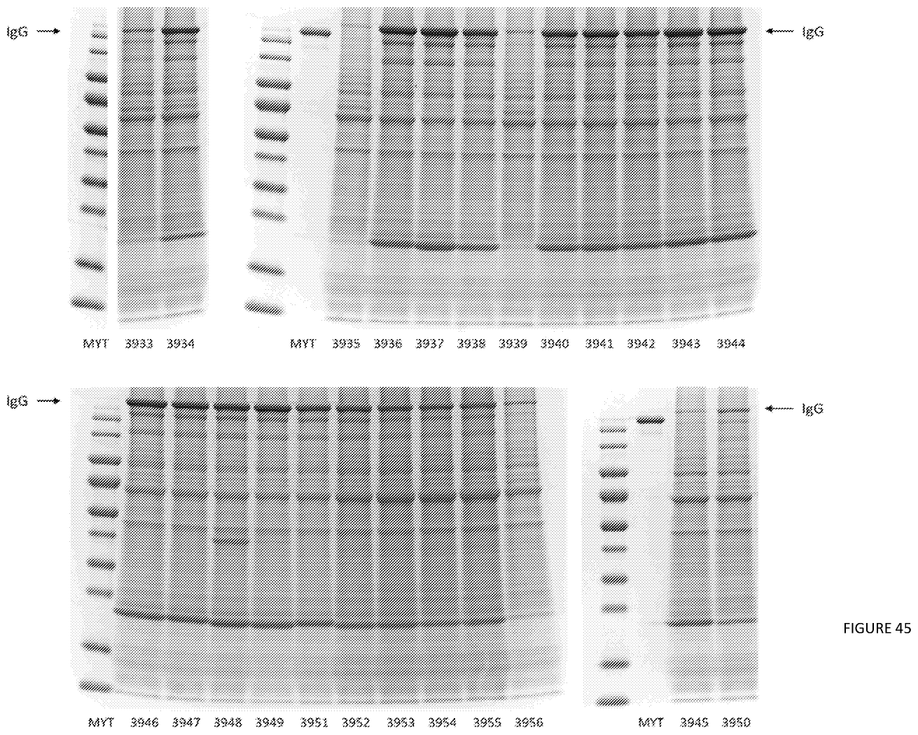

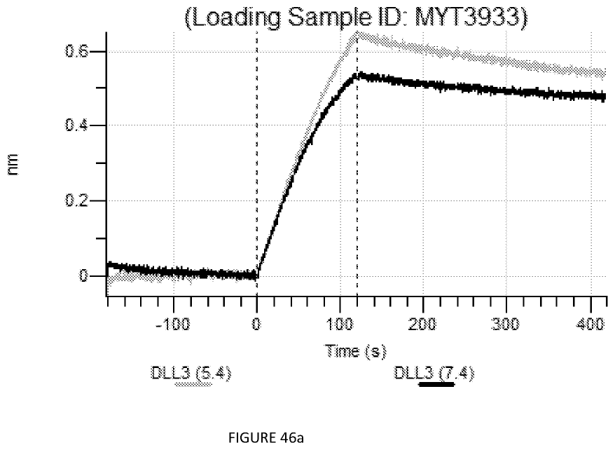

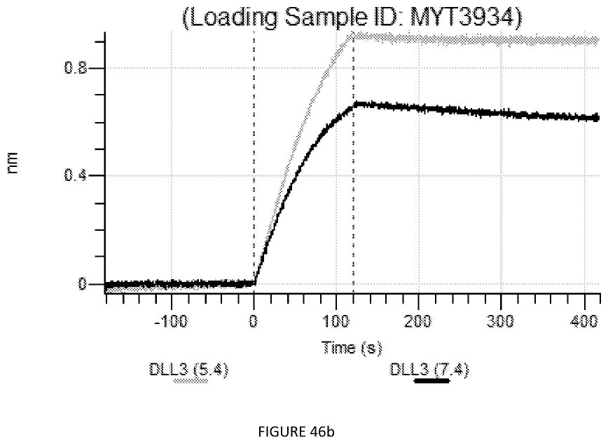

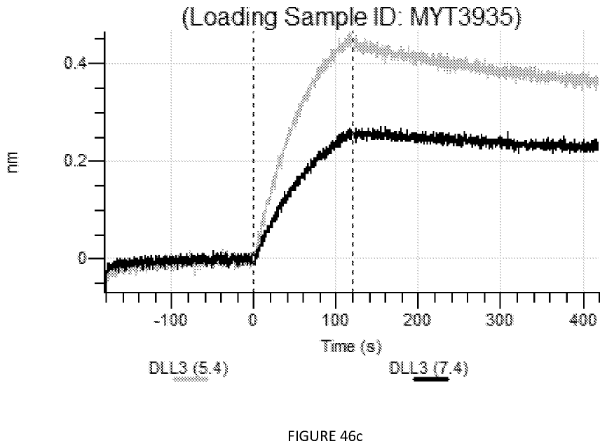

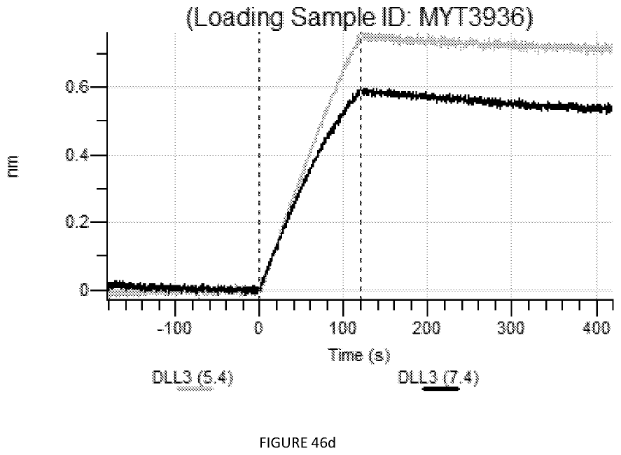

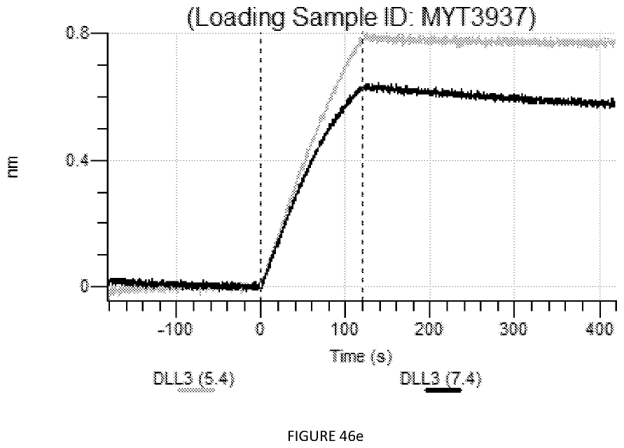

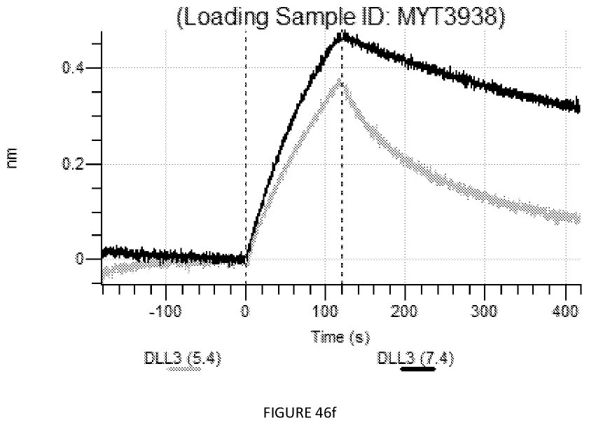

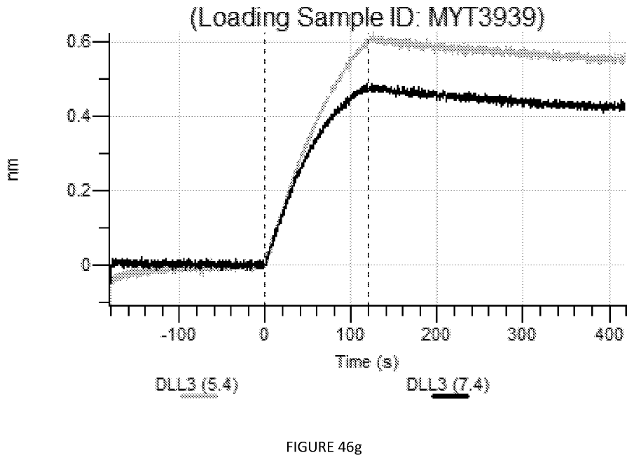

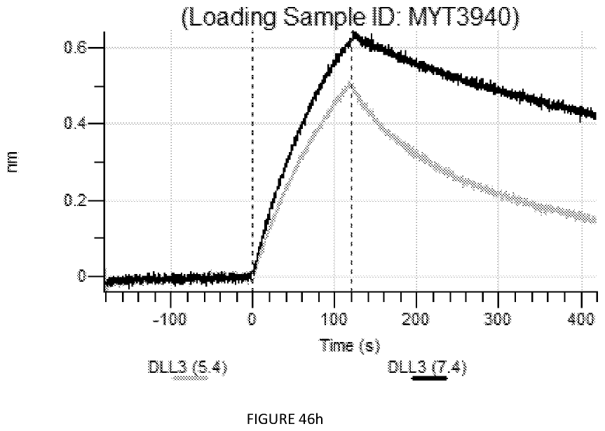

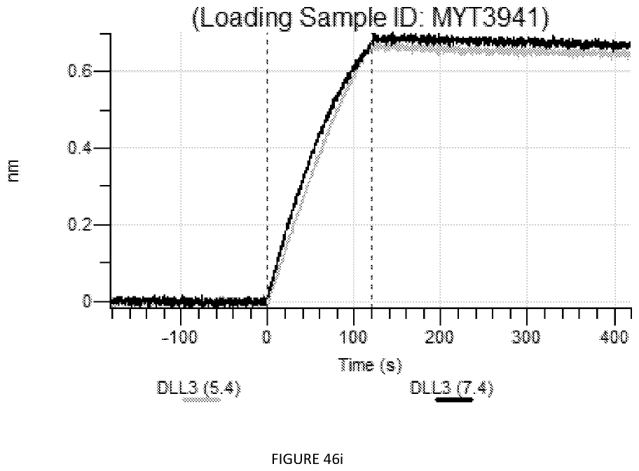

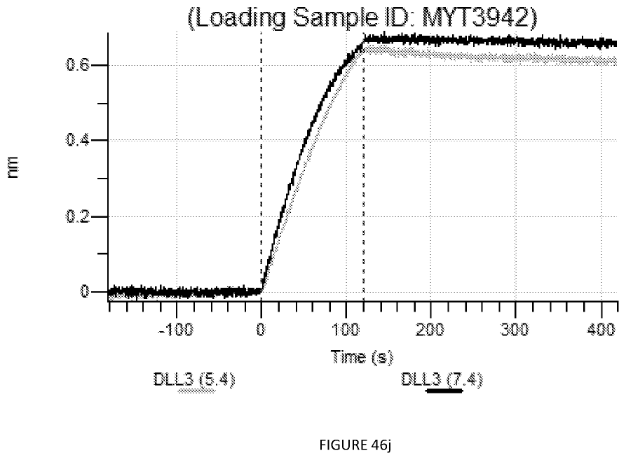

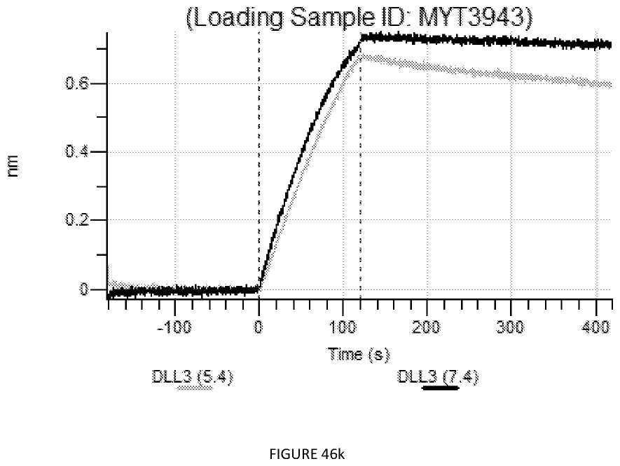

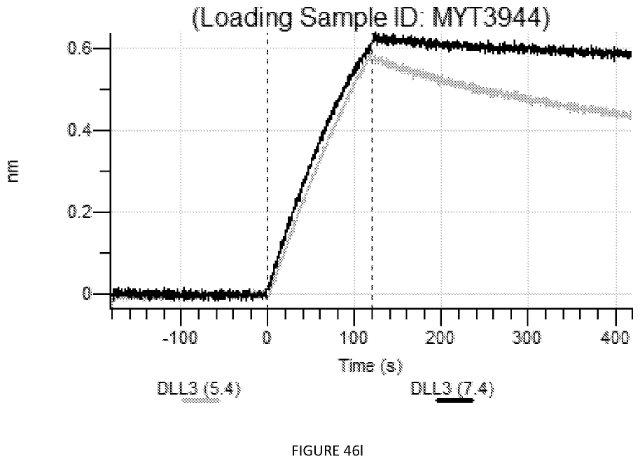

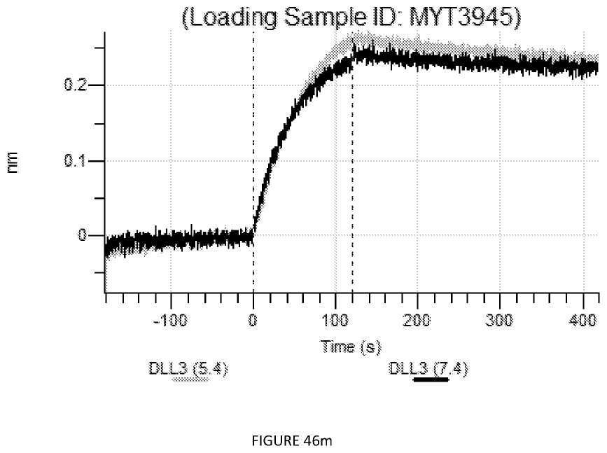

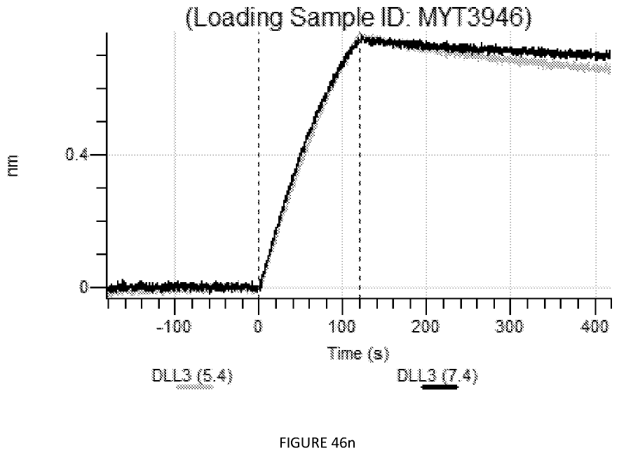

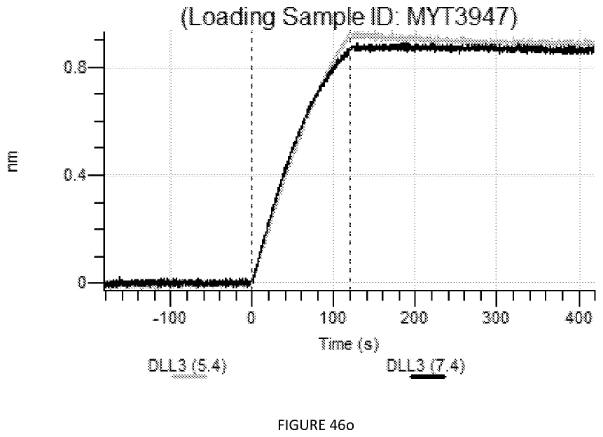

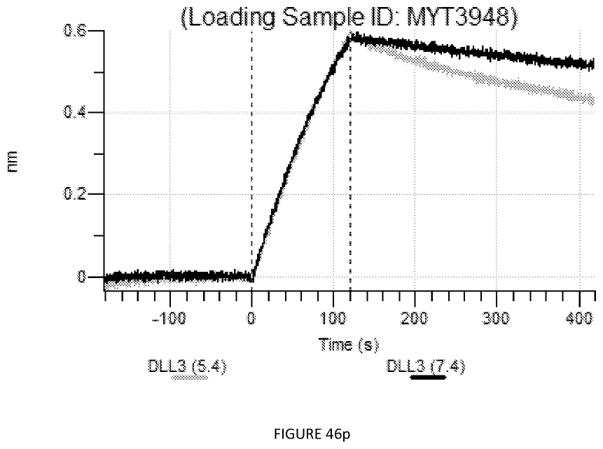

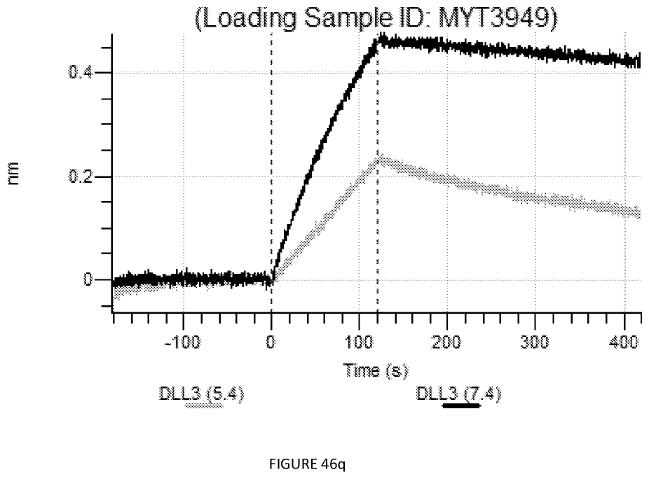

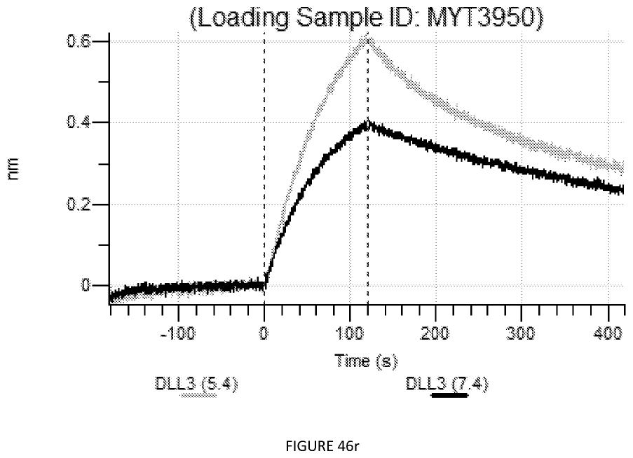

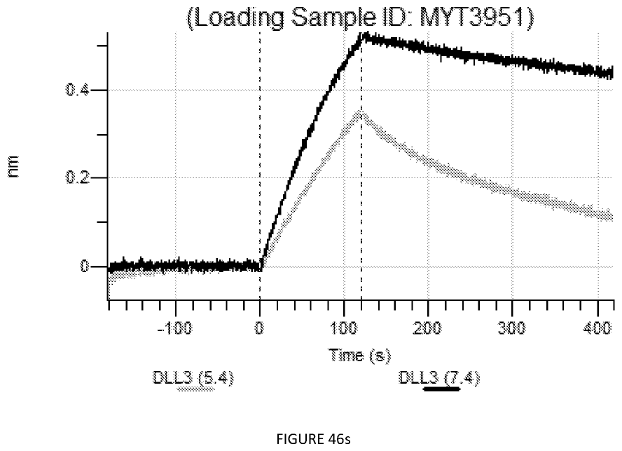

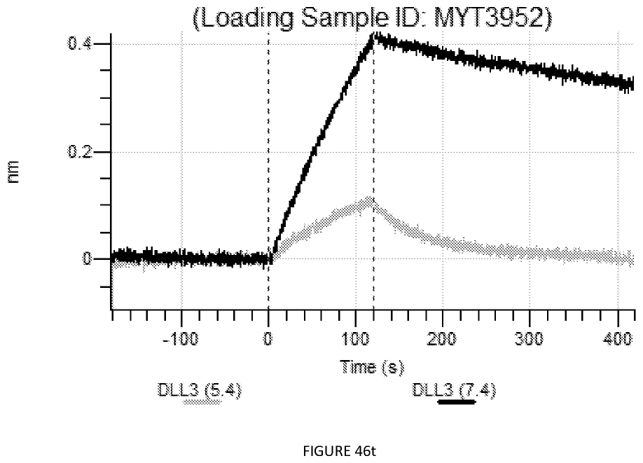

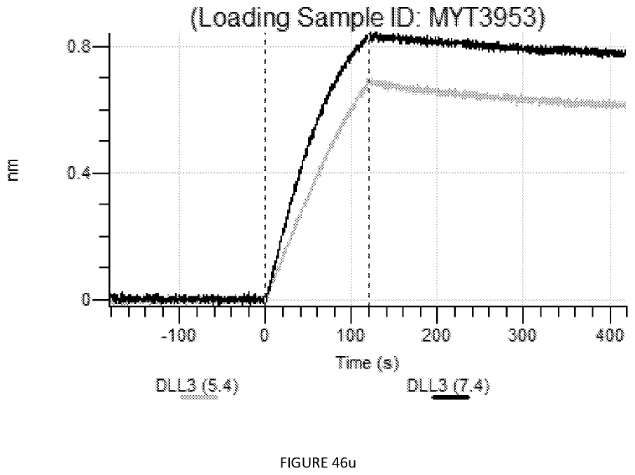

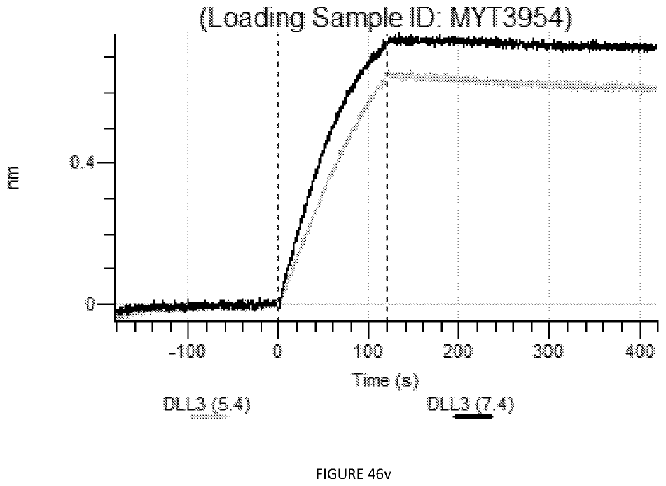

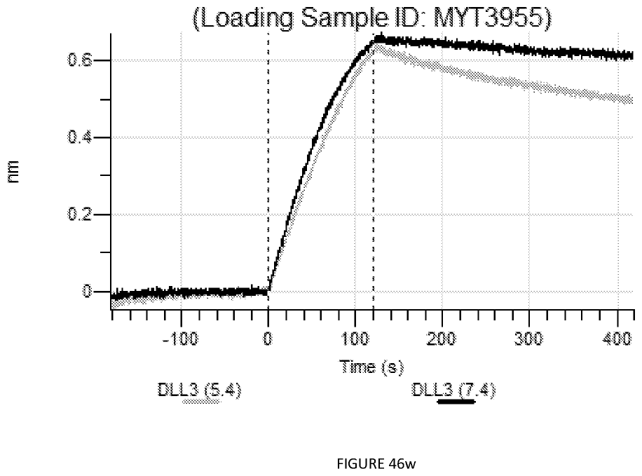

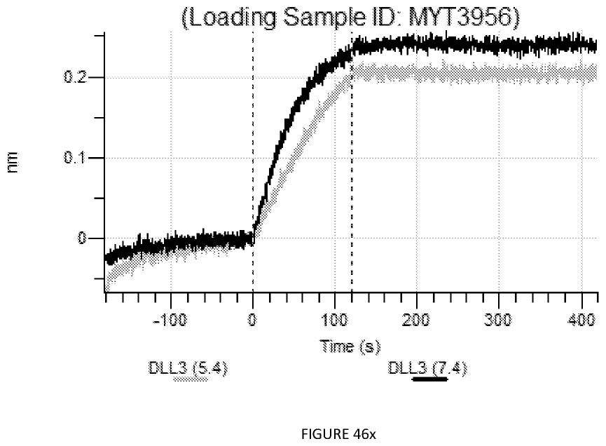

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.15 (U.S. Pat. No. 9,089,617 B2) (Heavy chain SEQ ID NO: 335, Light chain SEQ ID NO: 336) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3933-MYT3956). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 45), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 46 were selected for further analysis (e.g., MYT3933, MYT3935, MYT3938, MYT3940, MYT3943, MYT3944, MYT3948-MYT3953, and MYT3955). It was also noted that while no histidine or alanine mutations obliterated DLL3 binding, others were tolerated with little (e.g., less than 1-fold change in dissociation constant KD or dissociation rate) or no change in DLL3 binding kinetics (e.g., MYT3934, MYT3936, MYT3937, MYT3939, MYT3941, MYT3942, MYT3945, MYT3946, MYT3947, MYT3954, and MYT3956). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 26. Construction and Screening of pH-Engineered DLL3 ABPCs

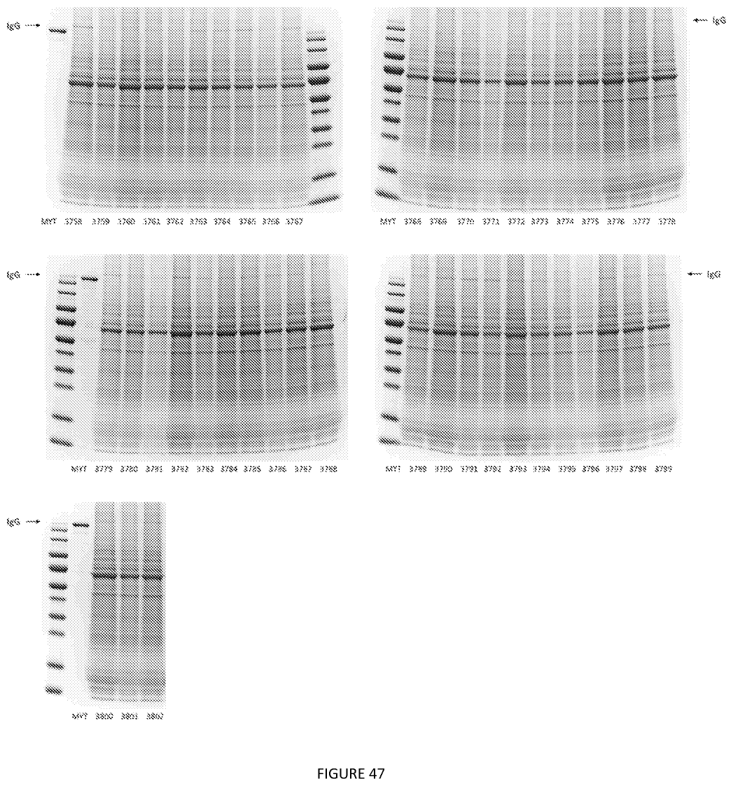

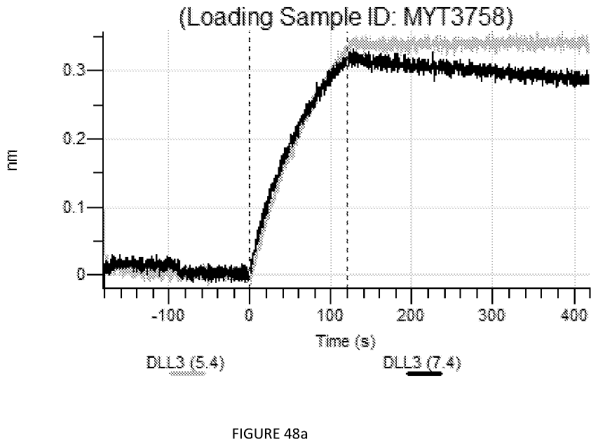

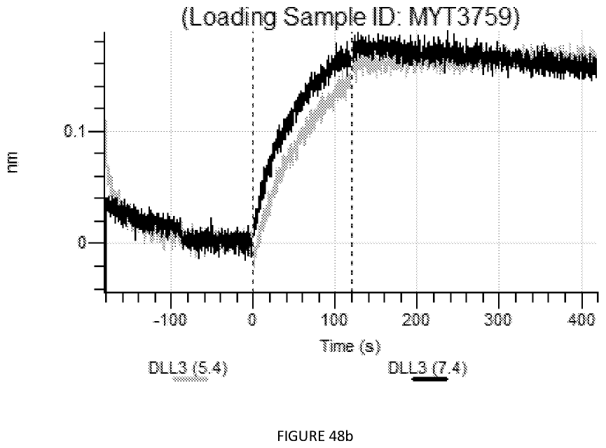

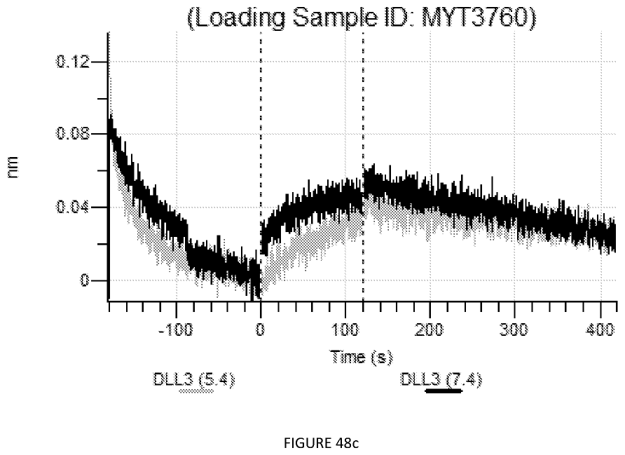

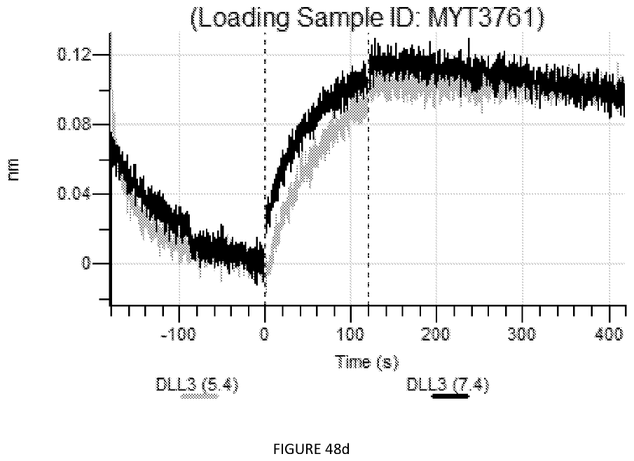

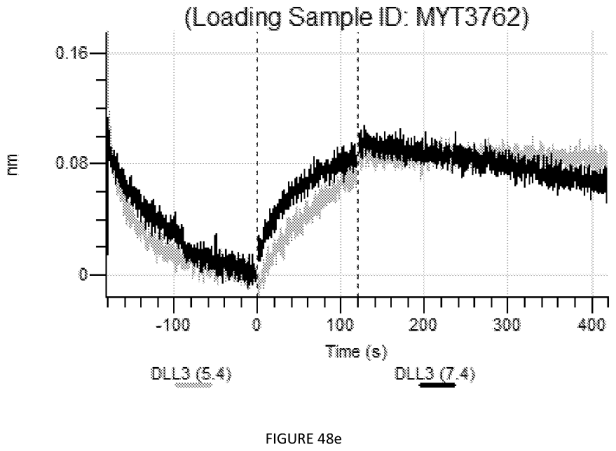

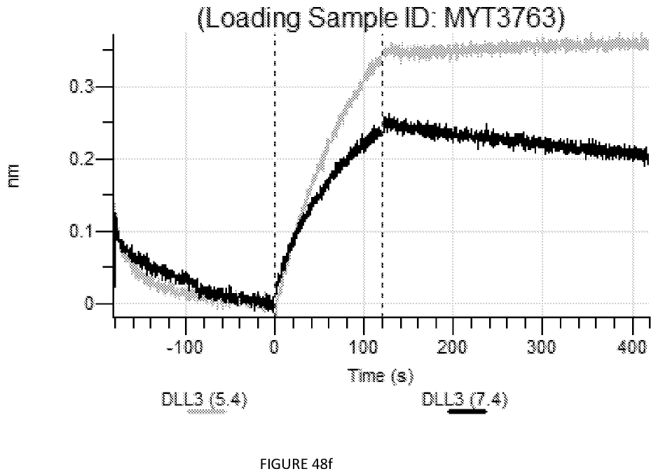

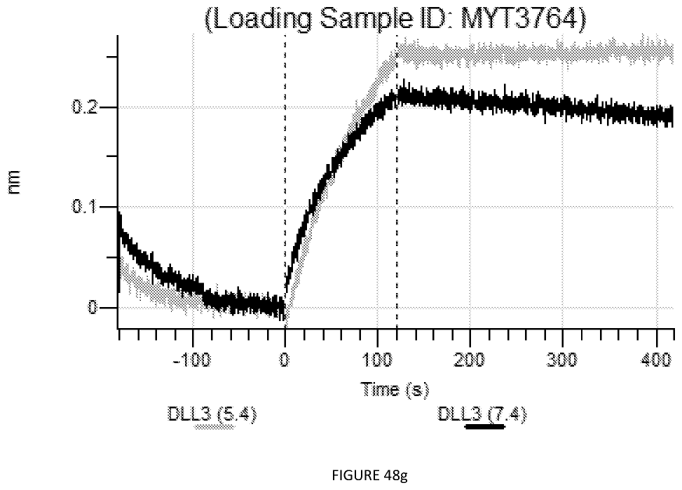

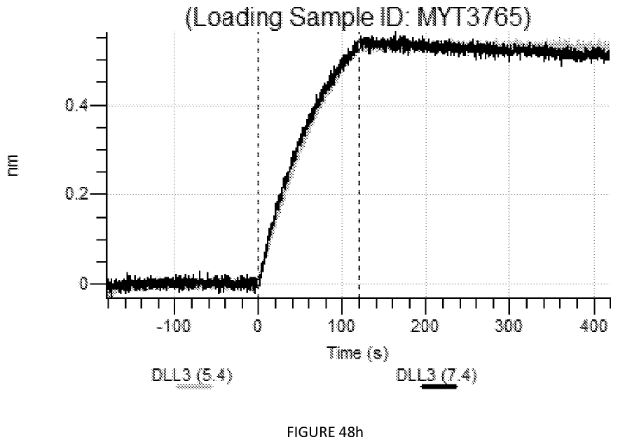

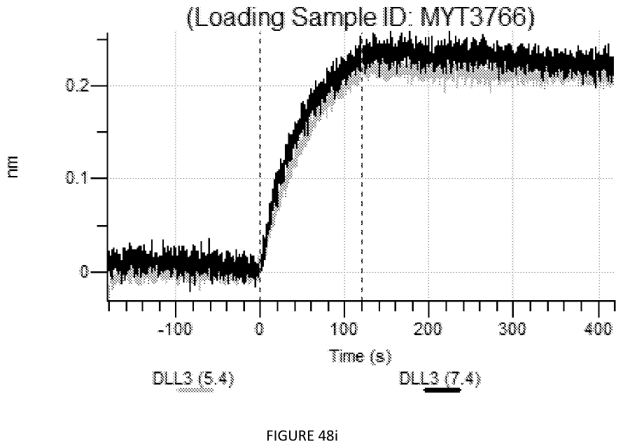

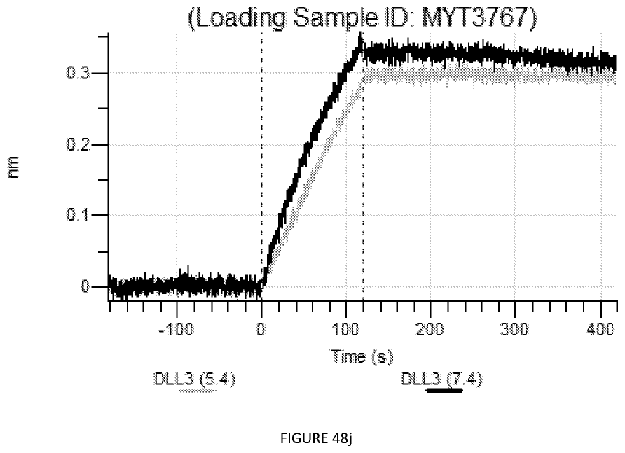

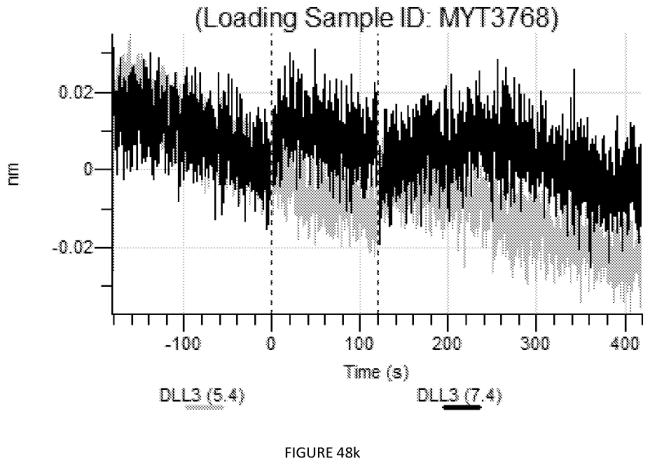

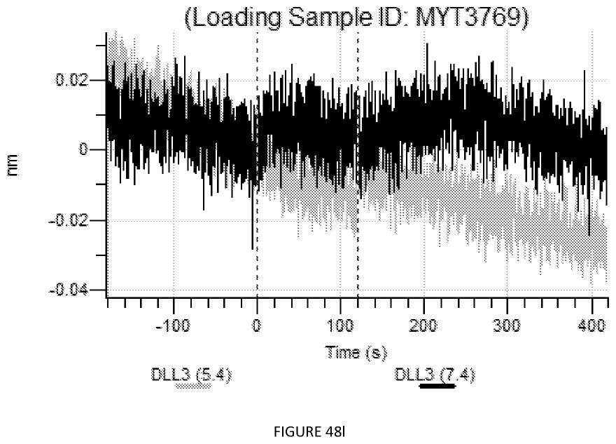

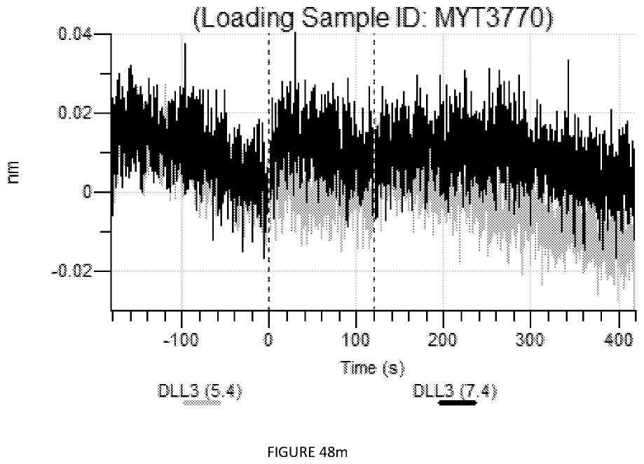

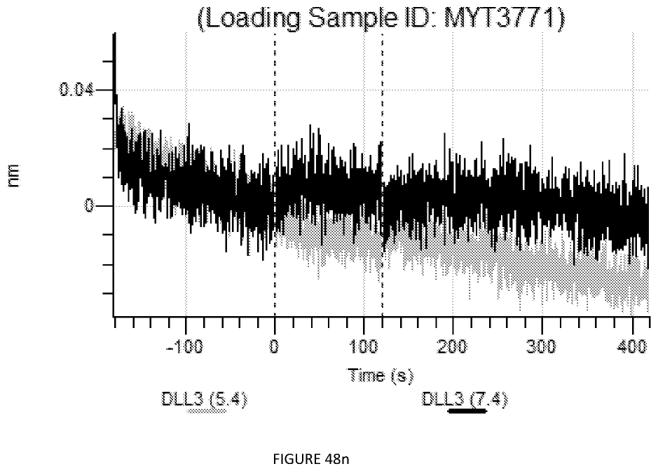

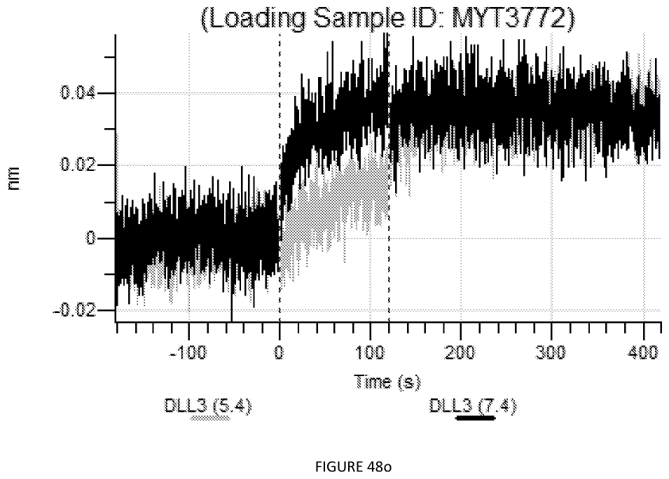

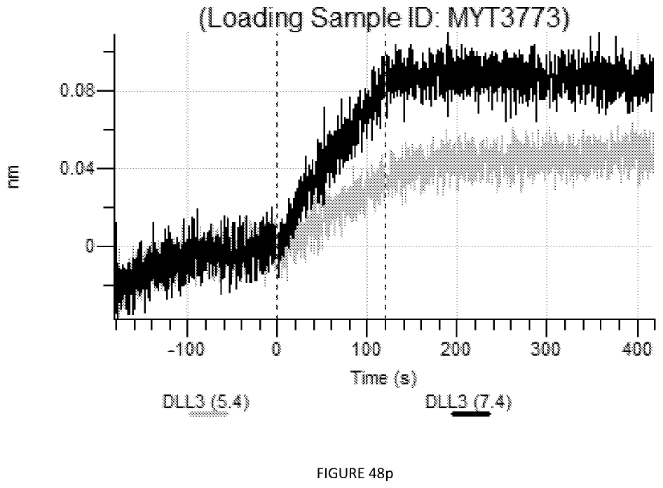

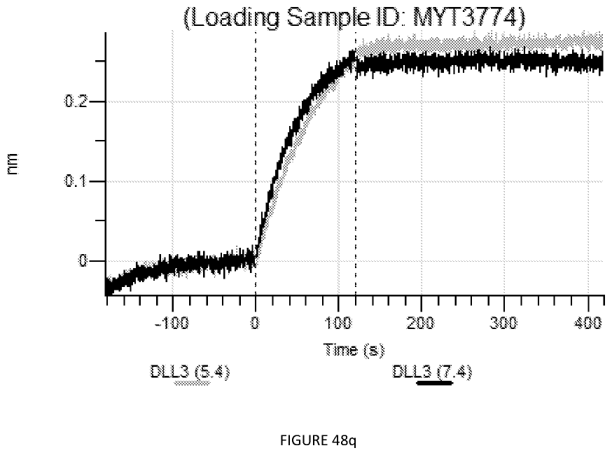

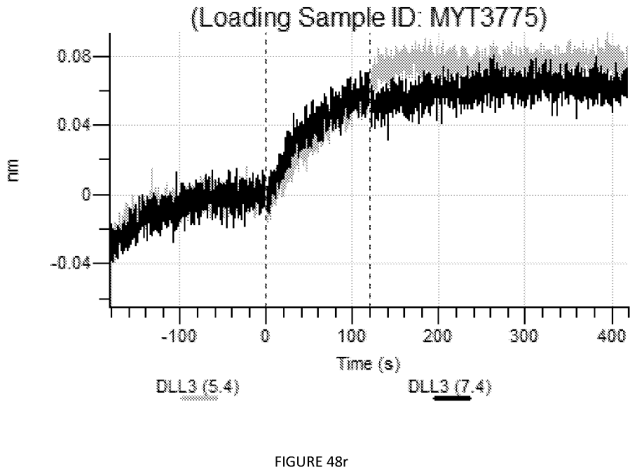

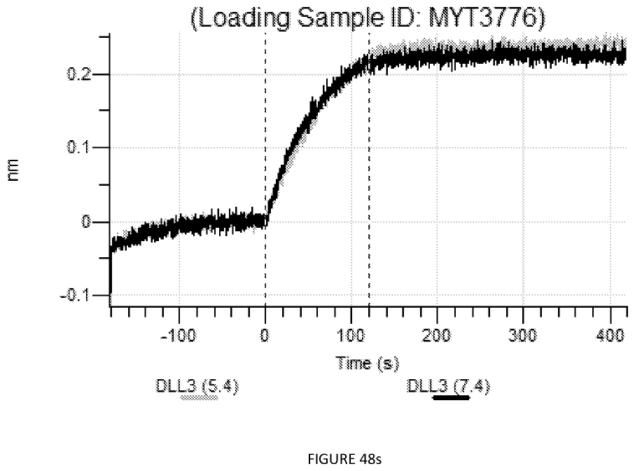

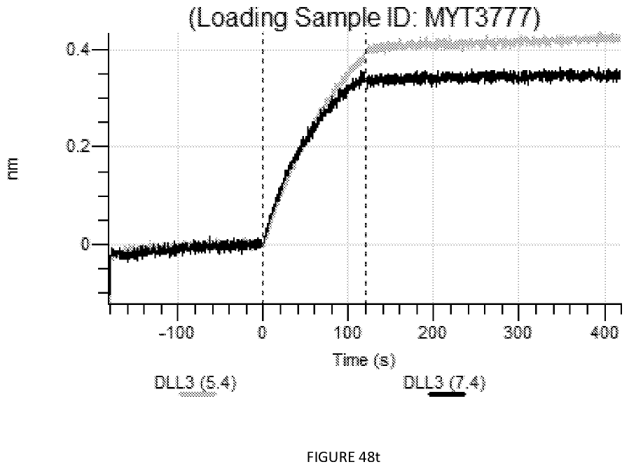

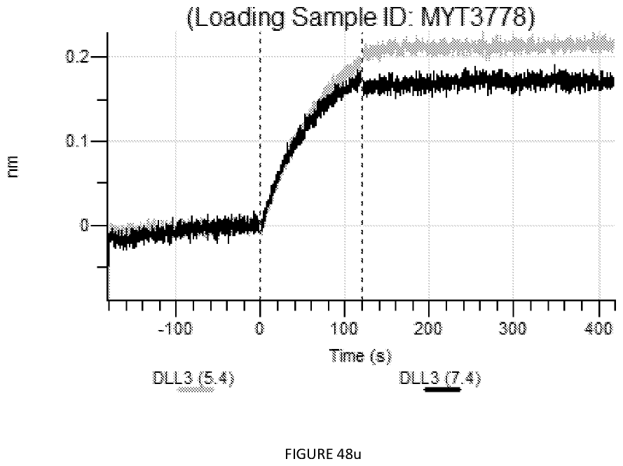

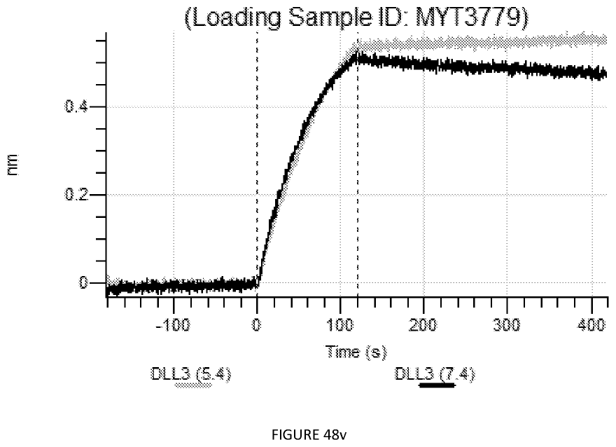

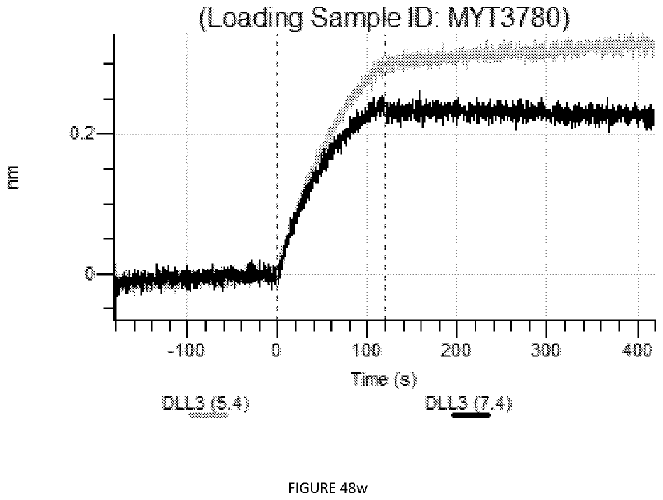

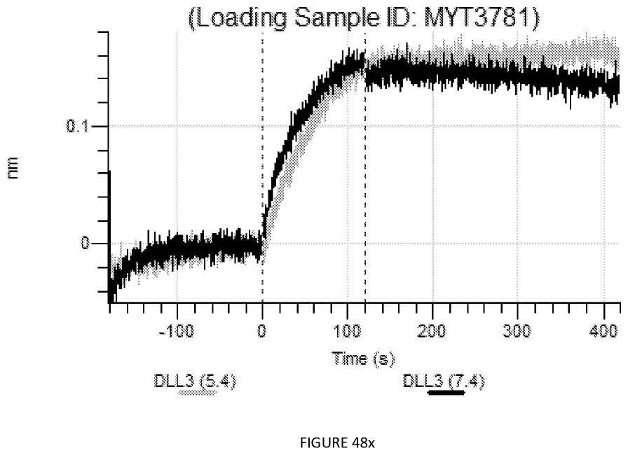

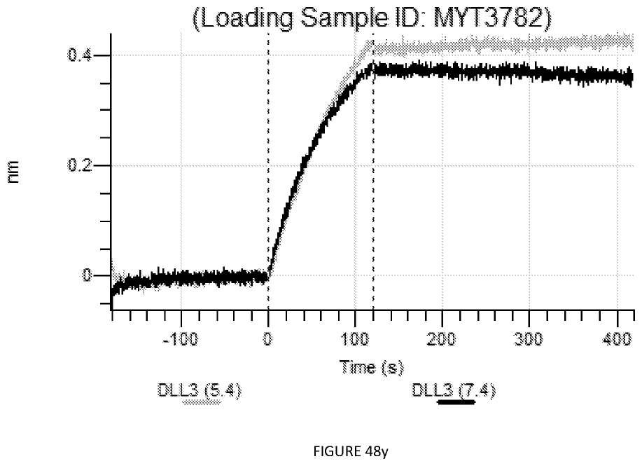

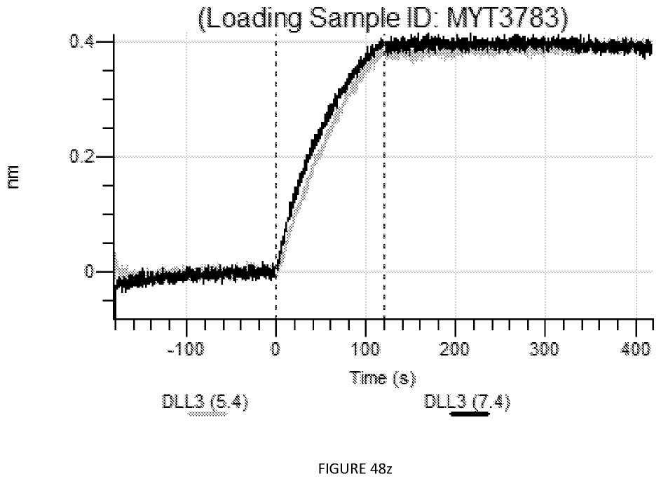

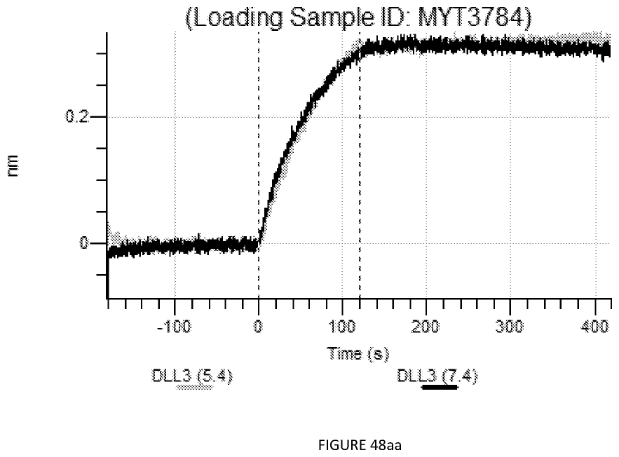

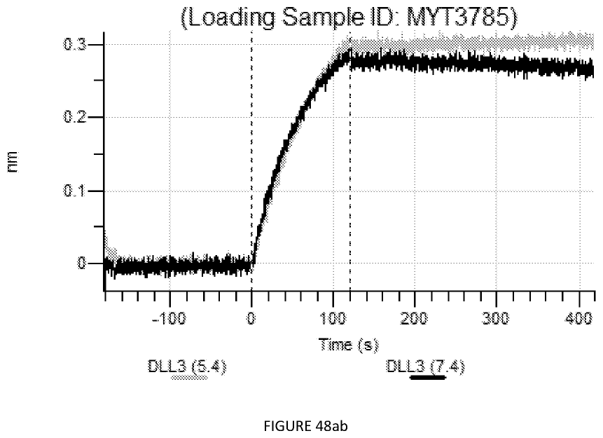

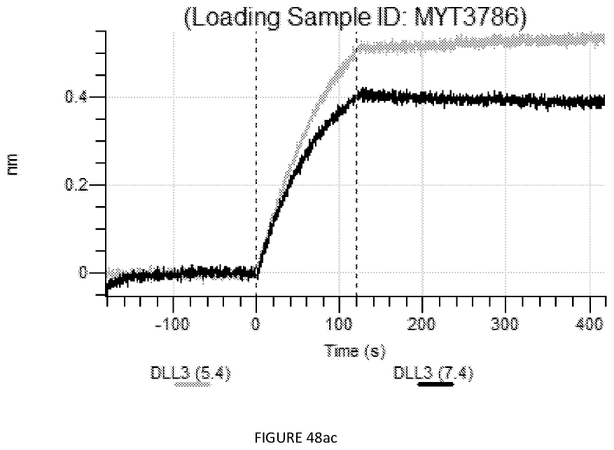

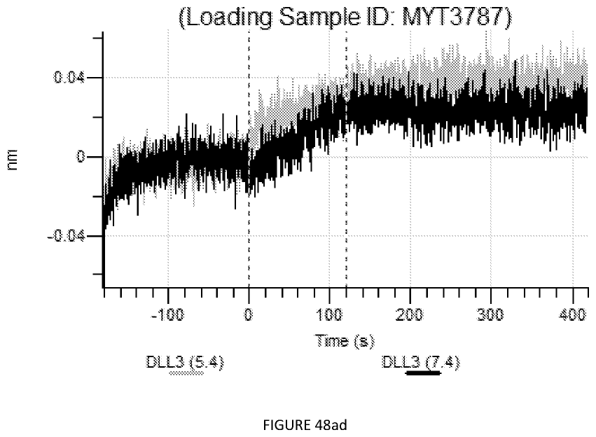

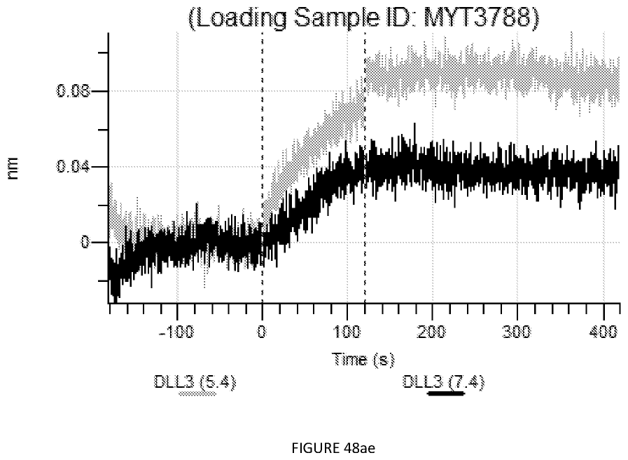

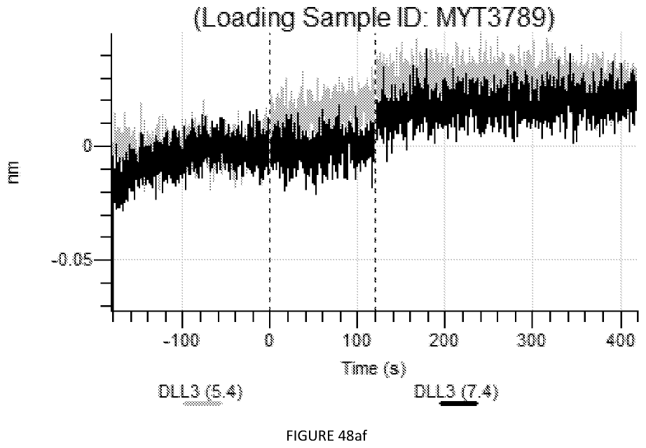

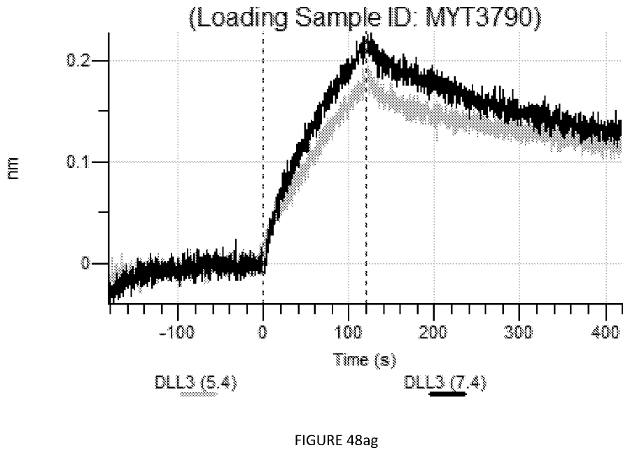

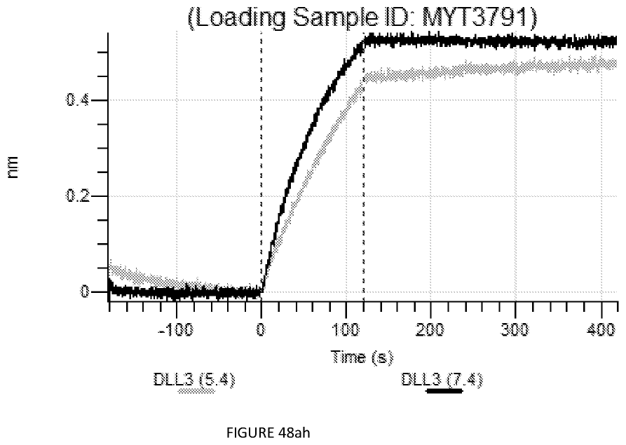

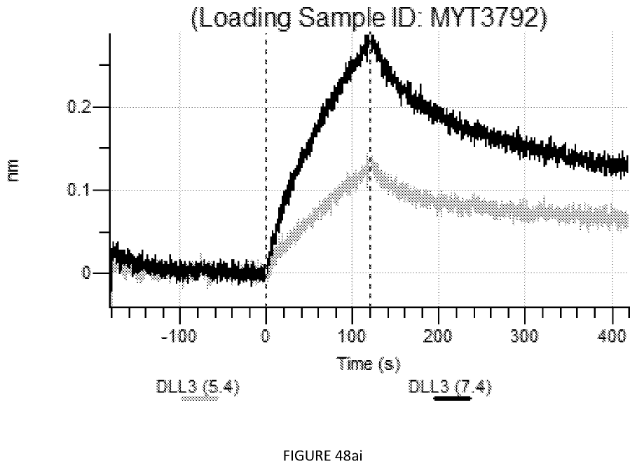

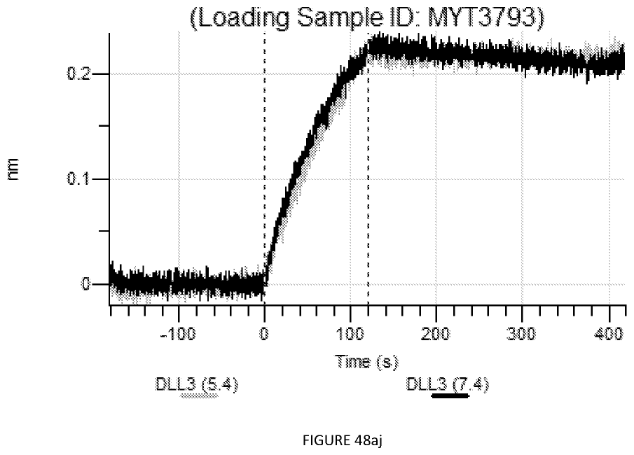

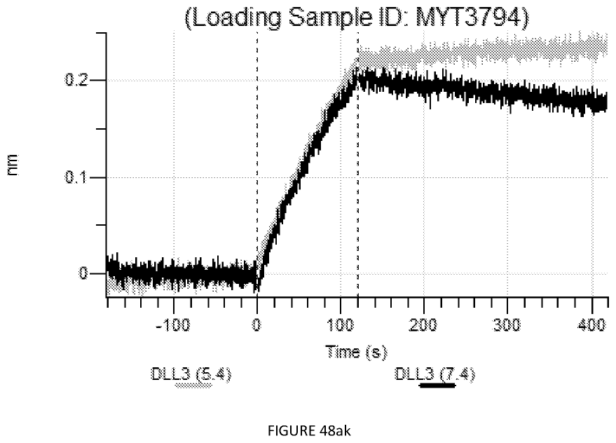

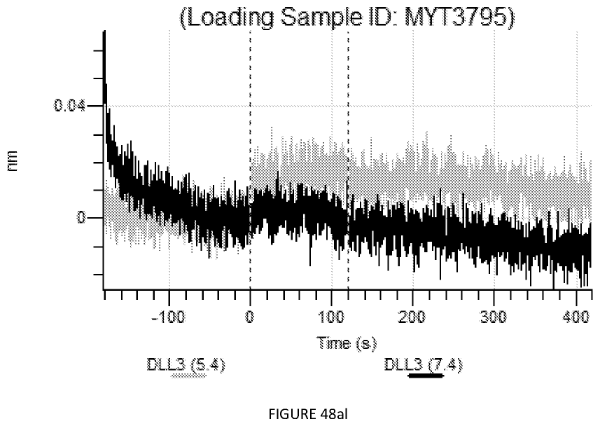

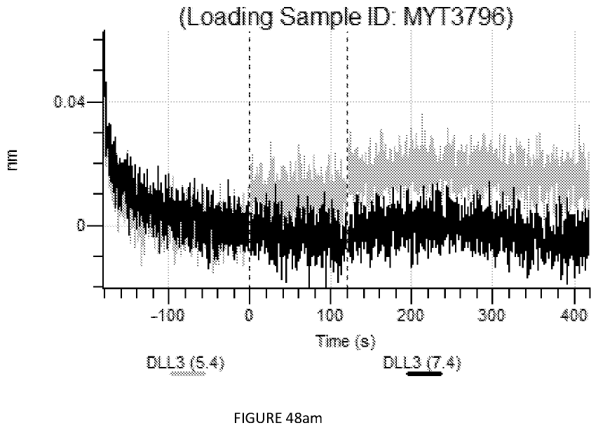

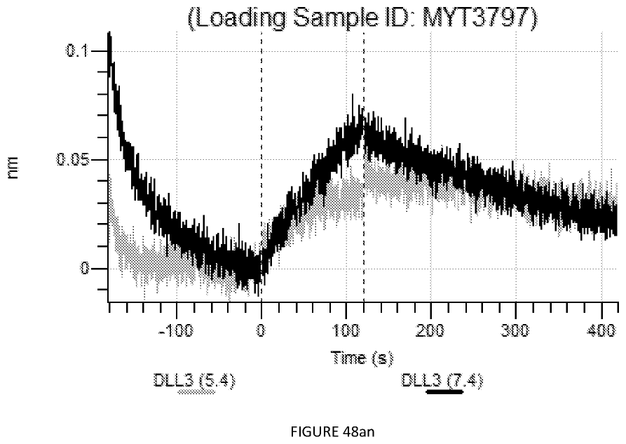

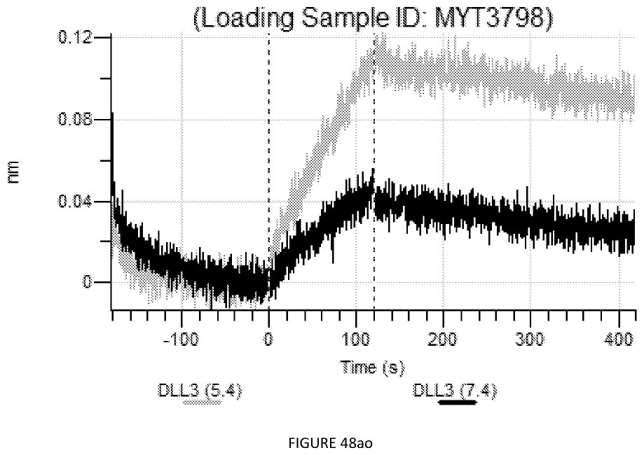

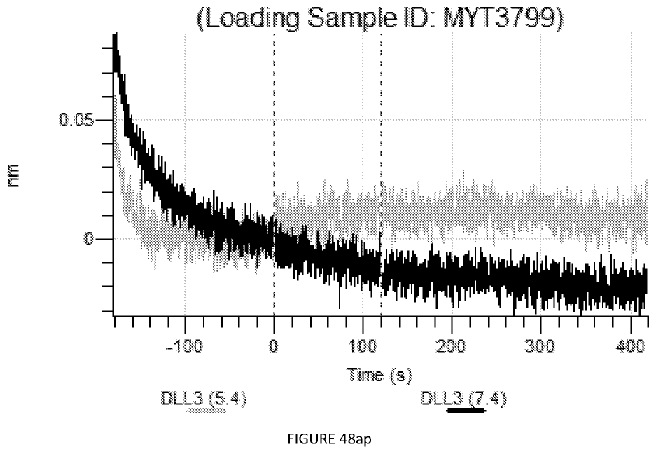

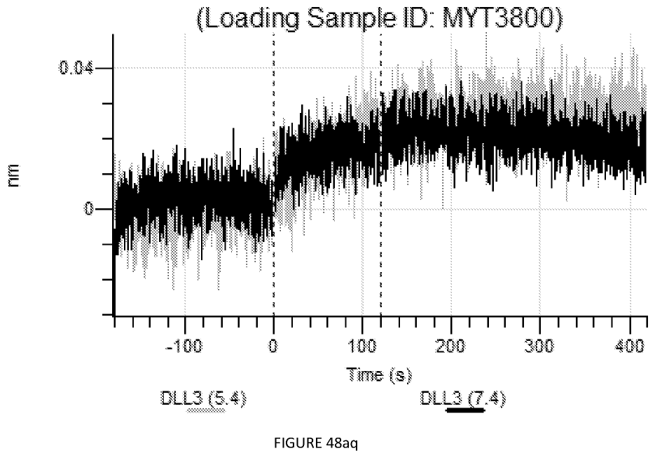

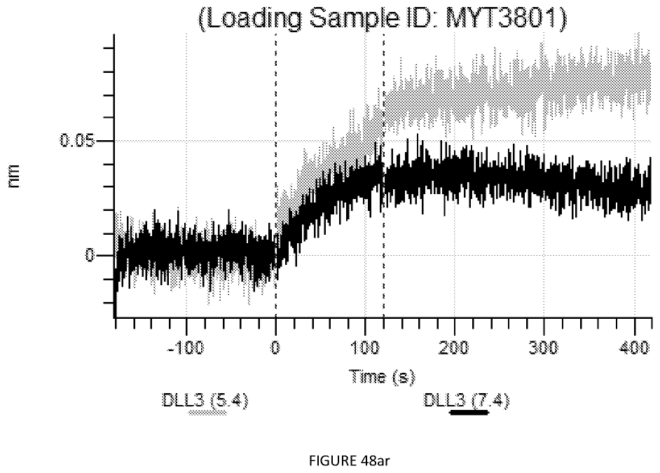

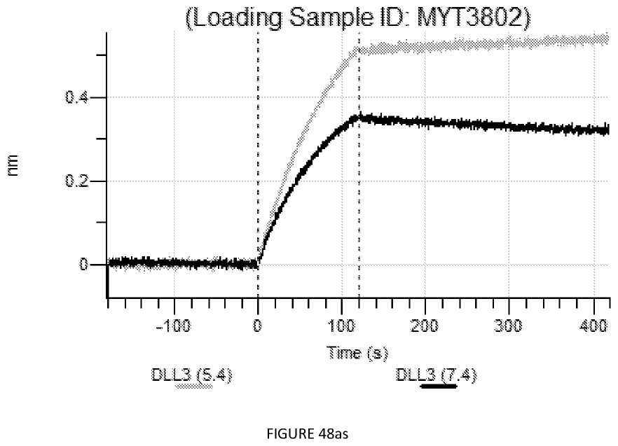

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.25 (U.S. Pat. No. 9,089,617 B2)(Heavy chain SEQ ID NO: 401, Light chain SEQ ID NO: 402) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3759-3802). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 47) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 48, were selected for further analysis (MYT3760, MYT3790, MYT3792, MYT3793, MYT3797, and MYT3798). It was also noted that while some histidine and alanine mutations obliterated DLL3 binding (e.g., MYT3789, MYT3795, MYT3799, and MYT3800), others were tolerated with little (e.g., less than 1-fold change in dissociation constant KD or dissociation rate) or no change in DLL3 binding kinetics (e.g., MYT3759, MYT3761, MYT3762, MYT3763, MYT3764, MYT3765, MYT3766, MYT3767, MYT3772-3788, MYT3791, MYT3794, MYT3801, and MYT3802). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 27. Construction and Screening of pH-Engineered DLL3 ABPCs

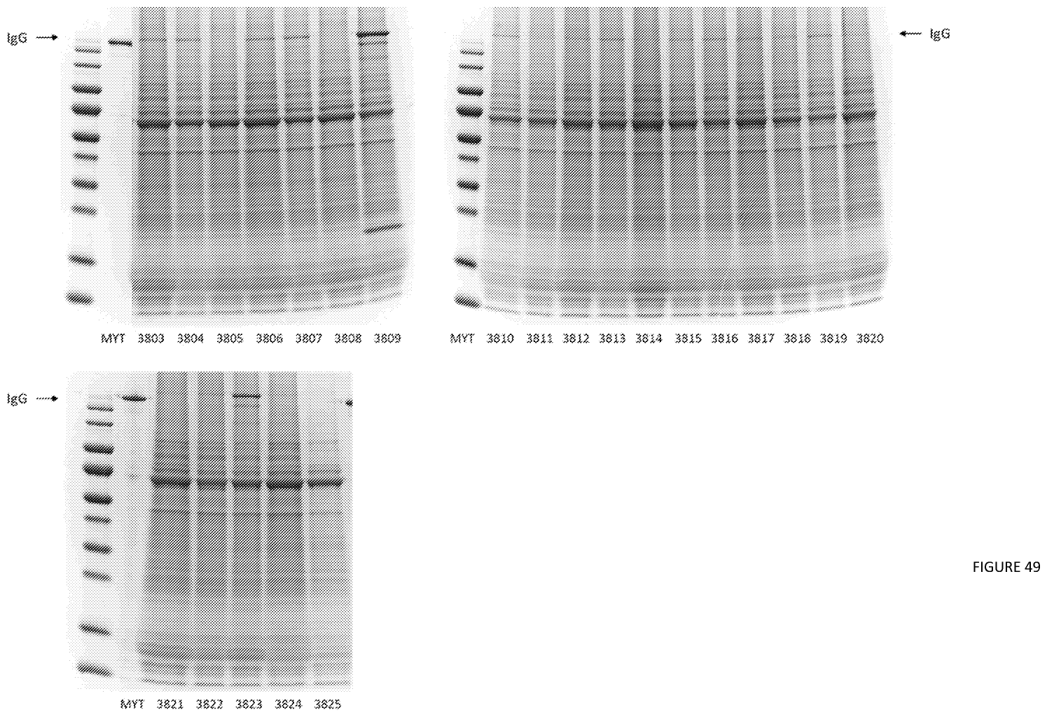

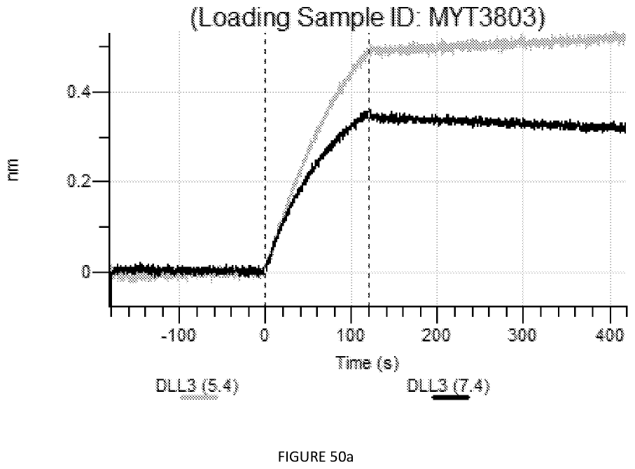

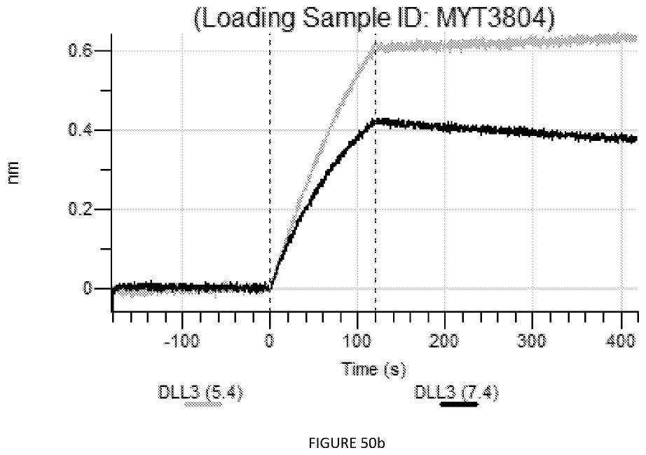

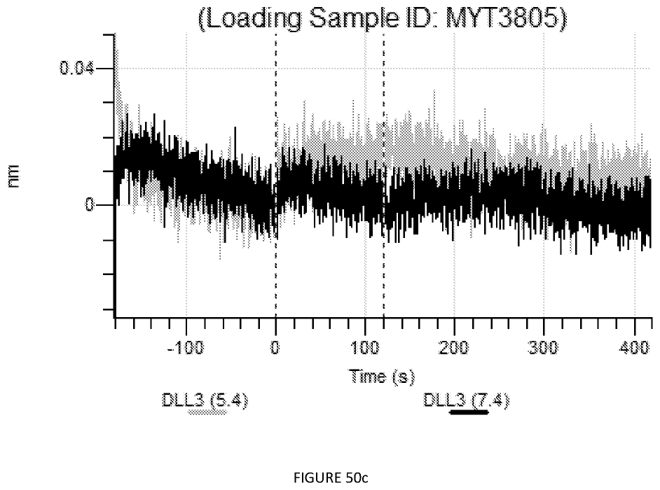

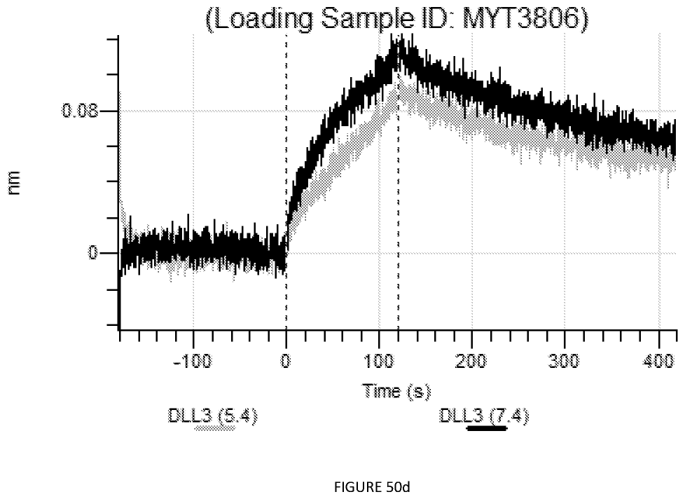

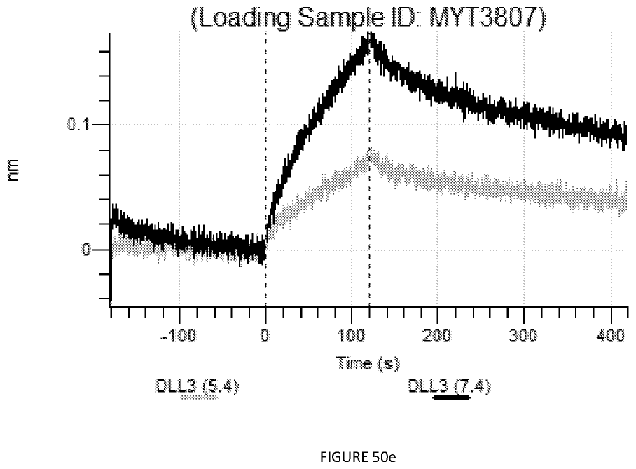

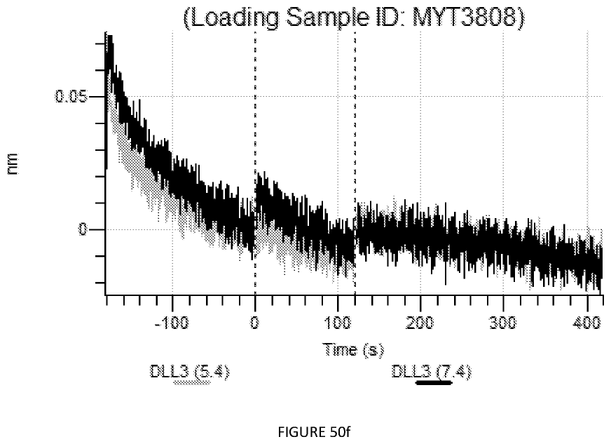

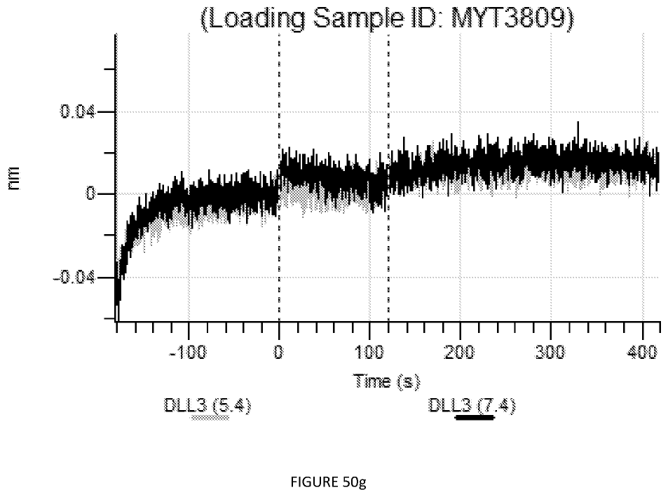

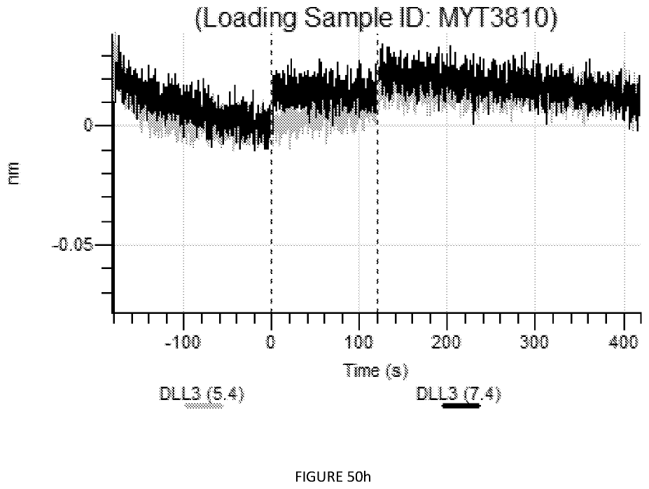

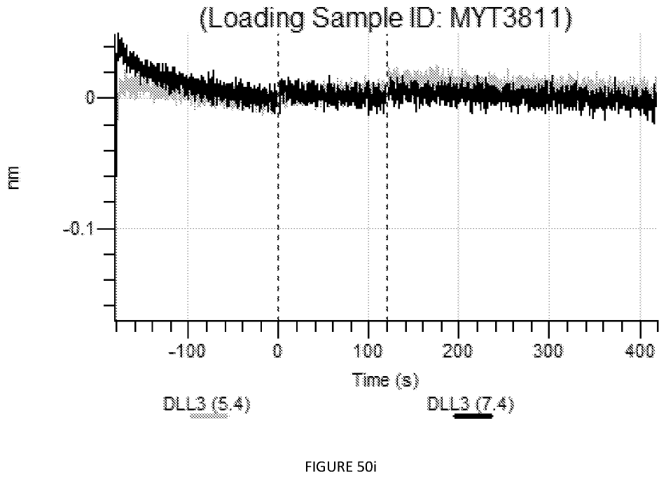

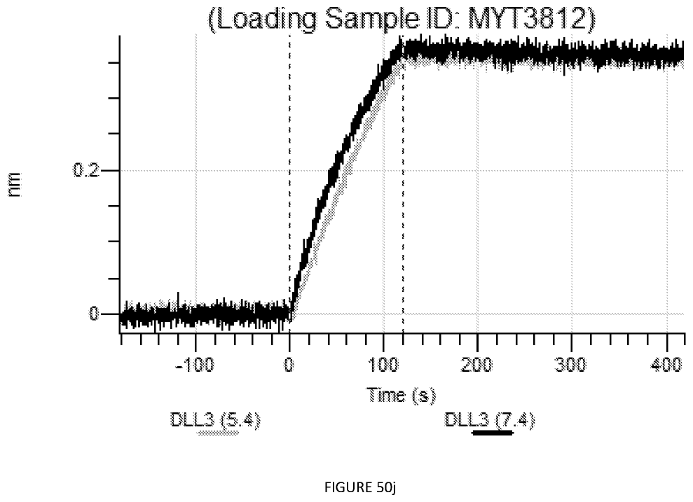

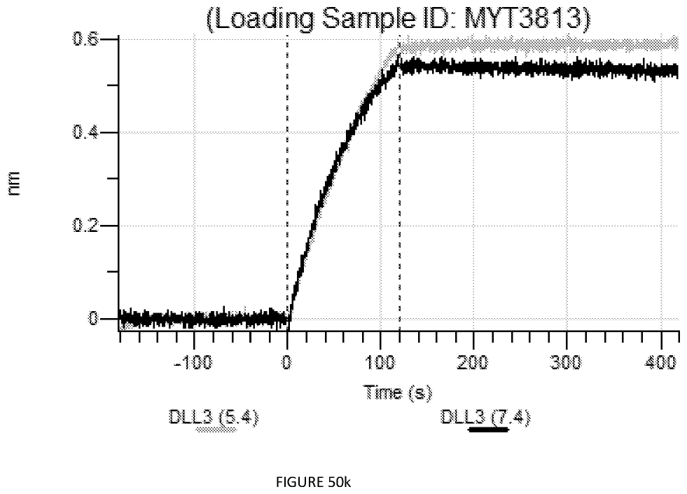

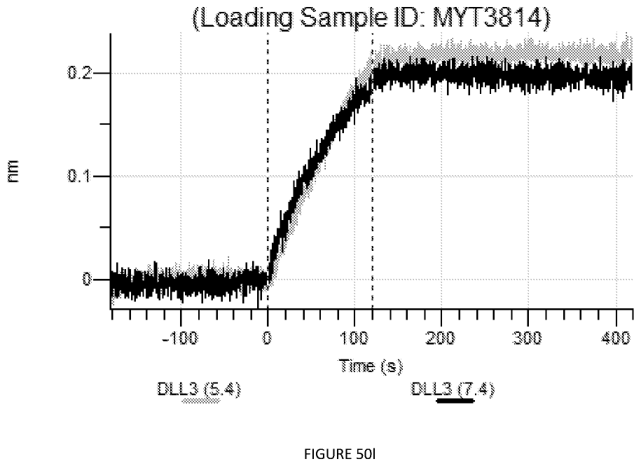

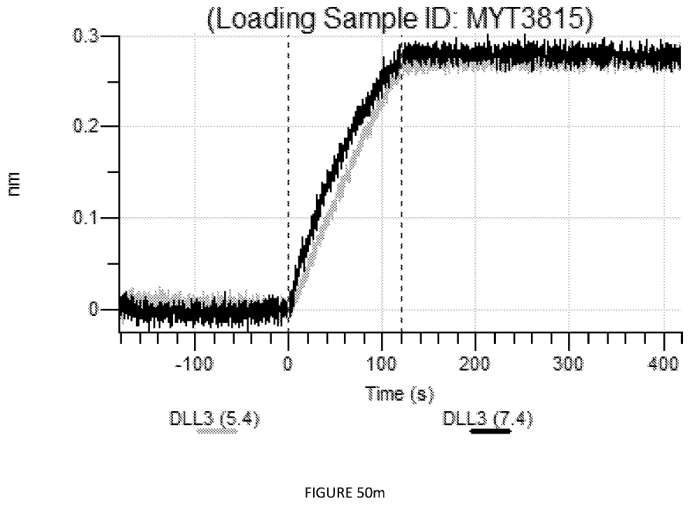

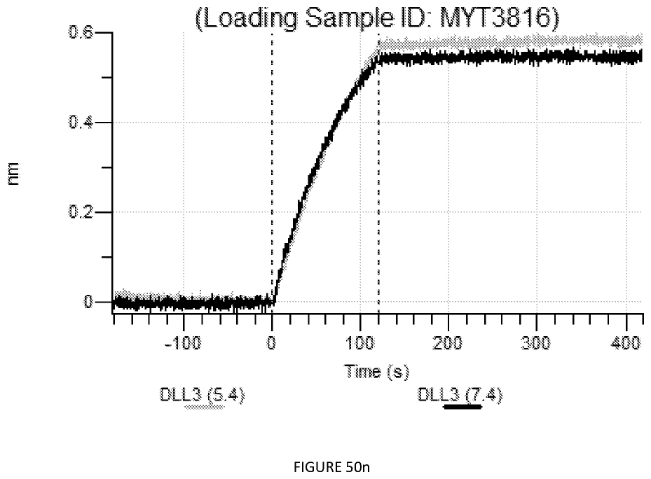

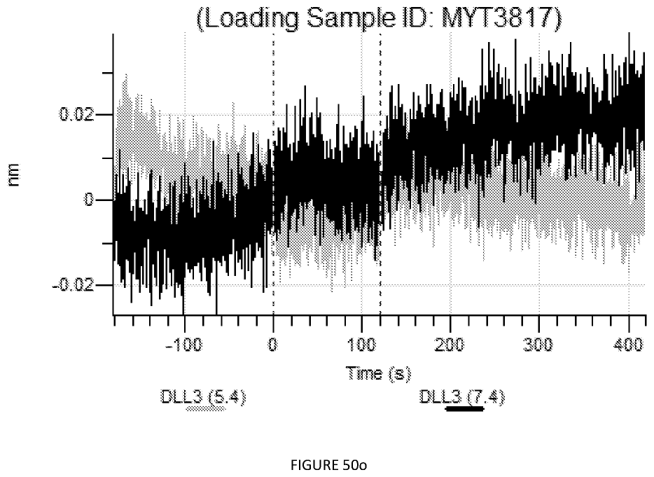

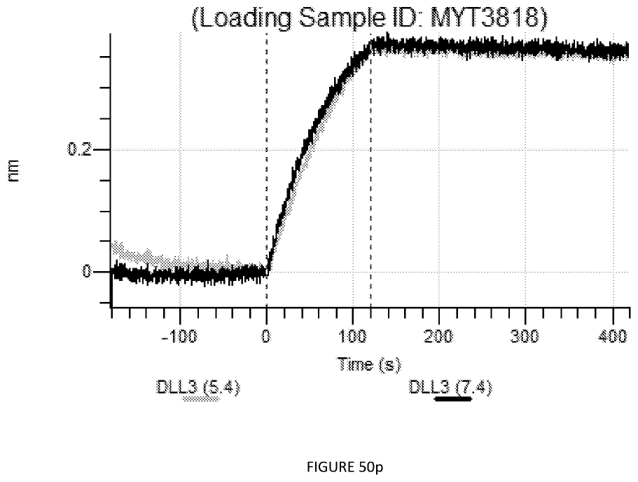

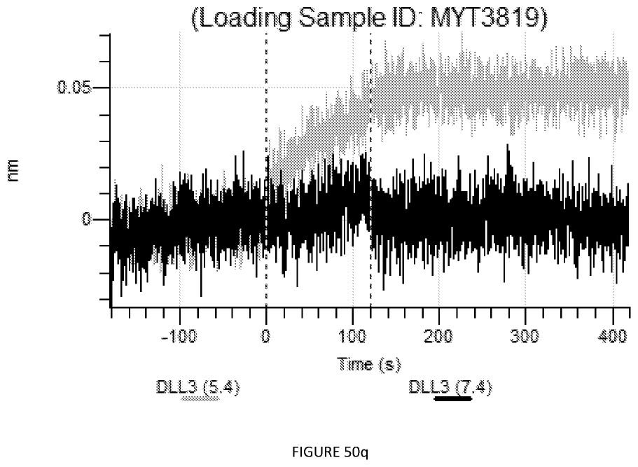

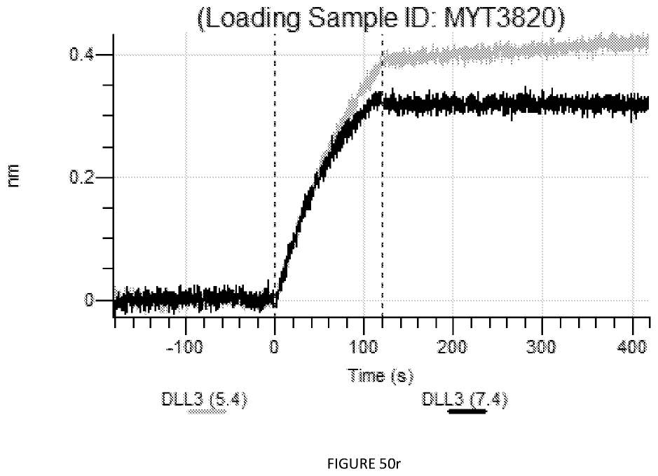

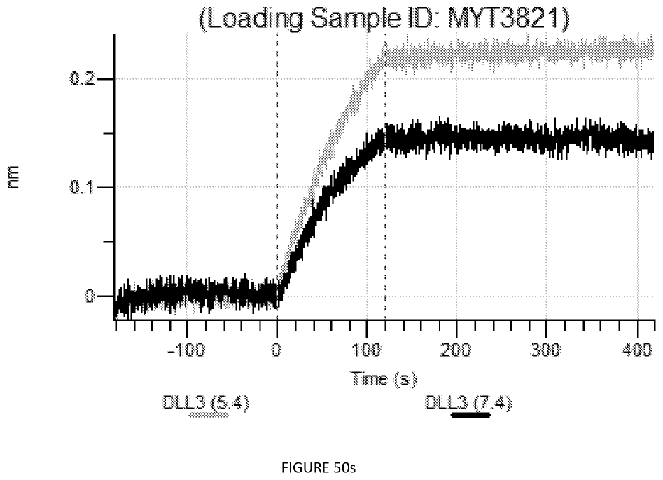

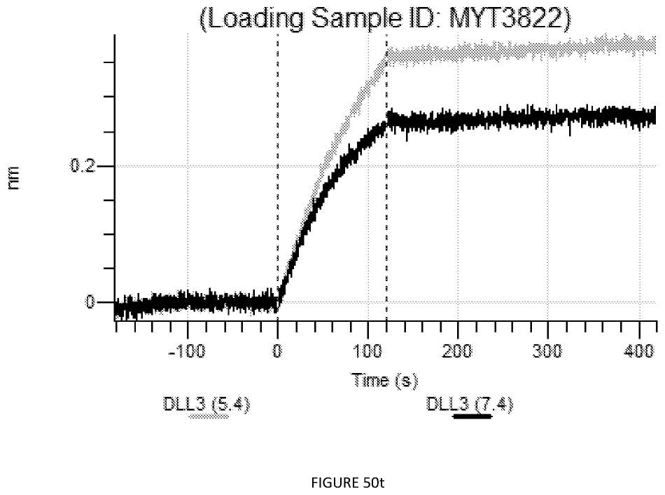

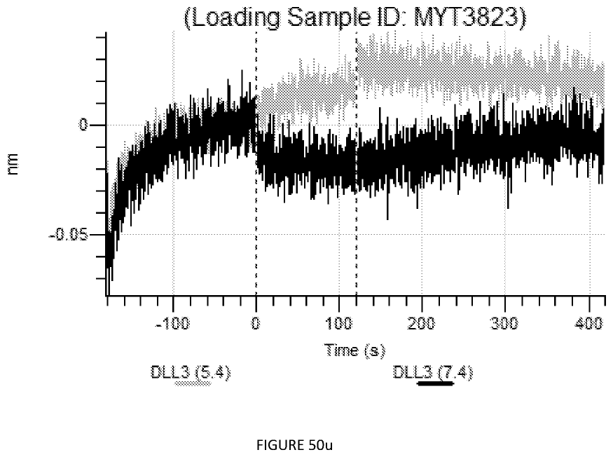

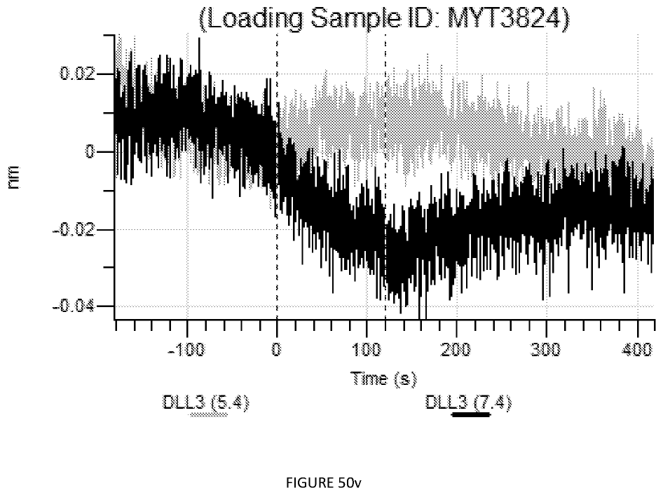

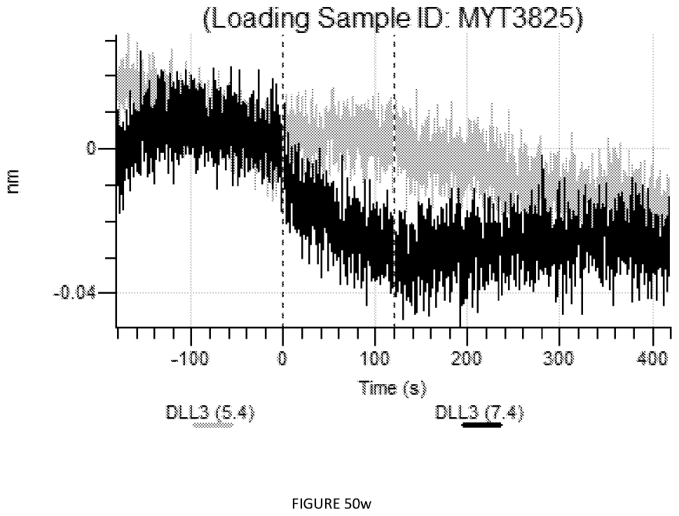

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.25 (U.S. Pat. No. 9,089,617 B2) (Heavy chain SEQ ID NO: 401, Light chain SEQ ID NO: 402) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3803-MYT3825). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 49), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 50, were selected for further analysis (e.g., MYT3806 and MYT3807). It was also noted that while some histidine and alanine mutations obliterated DLL3 binding (e.g., MYT3810, MYT3811, and MYT3817), others were tolerated with little (e.g., less than 1-fold change in dissociation constant KD or dissociation rate) or no change in DLL3 binding kinetics (e.g., MYT3803, MYT3804, MYT3812, MYT3813, MYT3814, MYT3815, MYT3816, MYT3818, MYT3820, MYT3821, and MYT3822). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 28. Construction and Screening of pH-Engineered DLL3 ABPCs

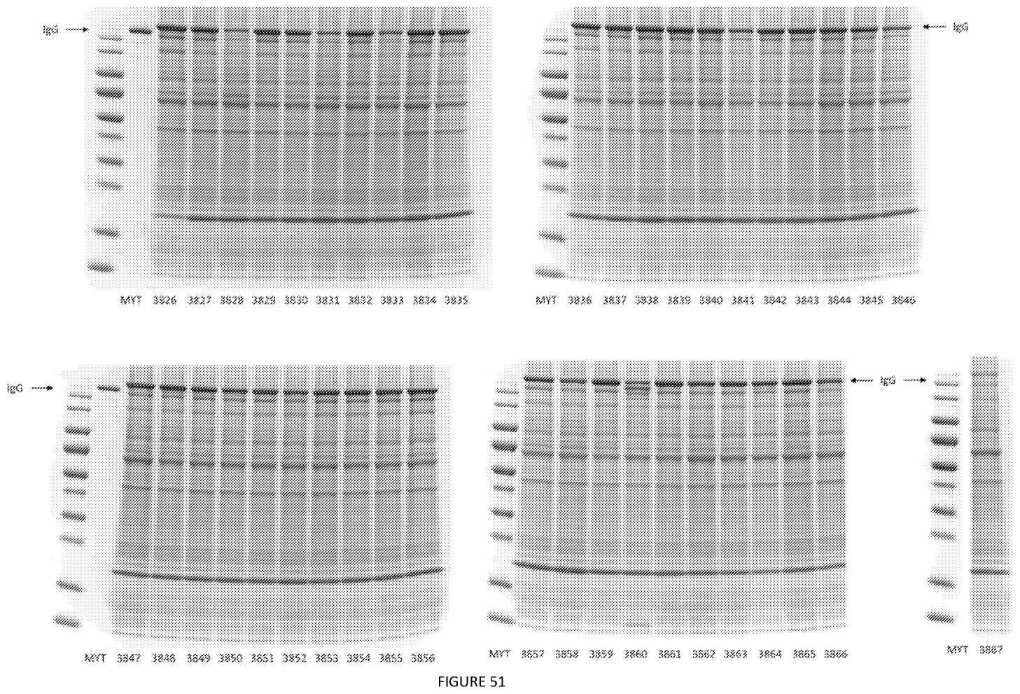

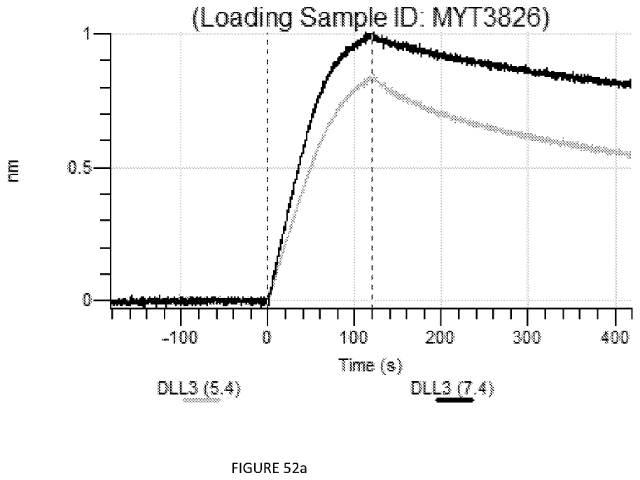

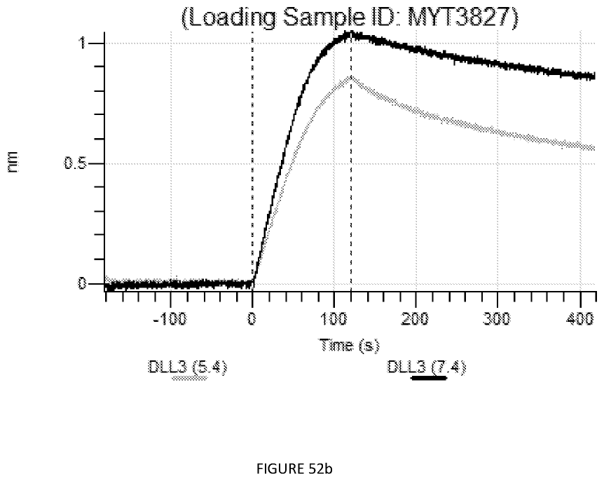

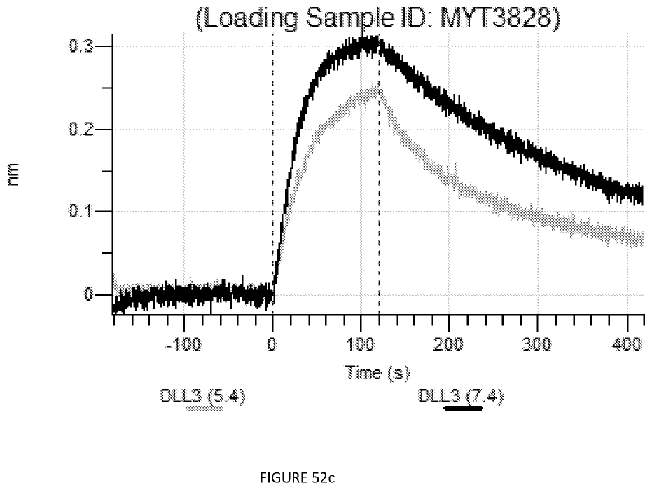

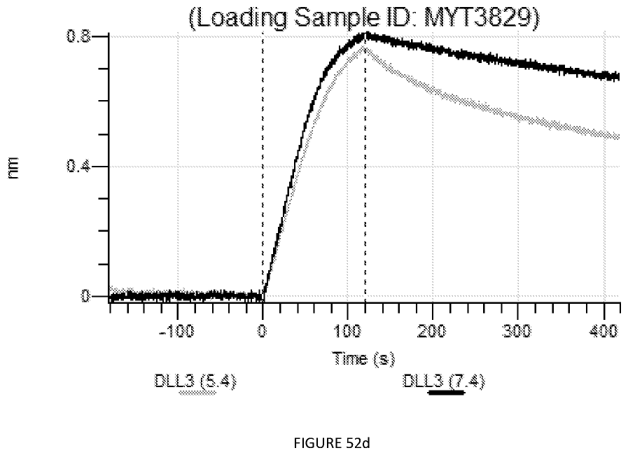

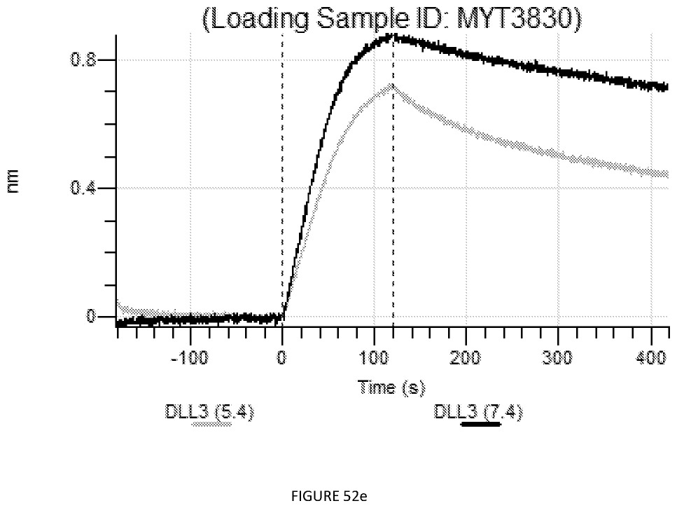

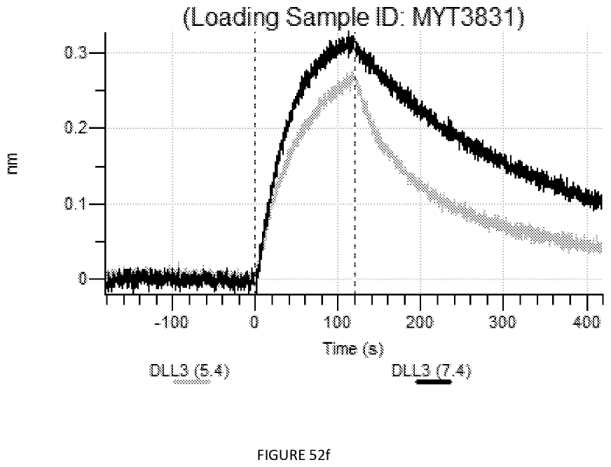

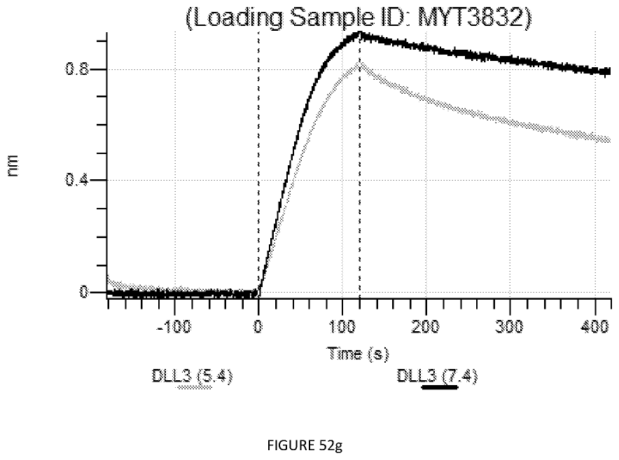

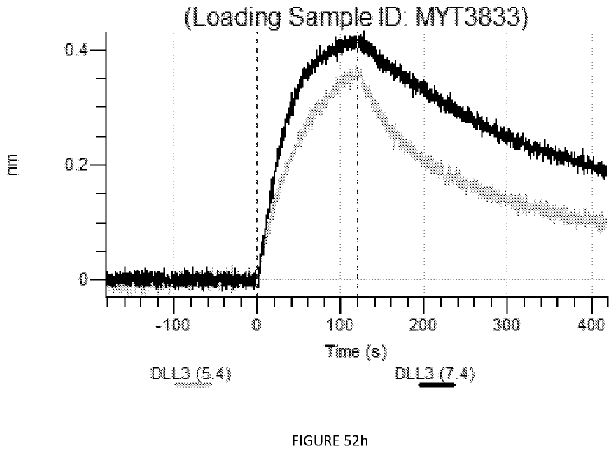

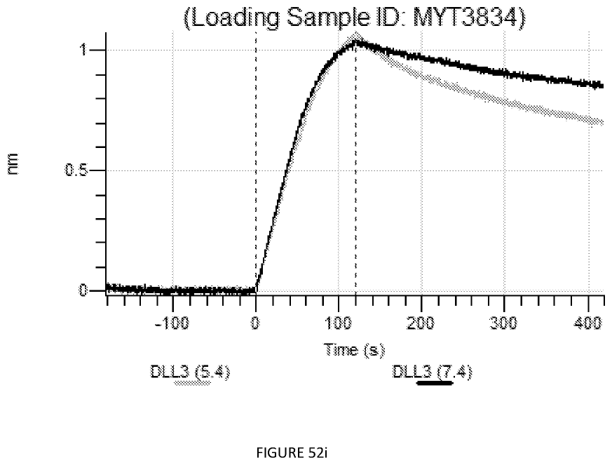

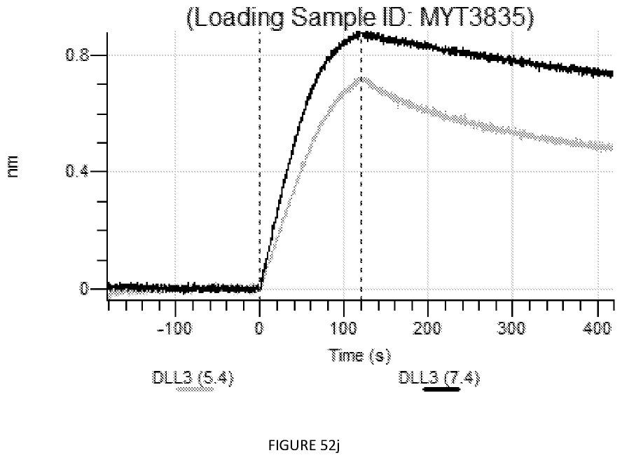

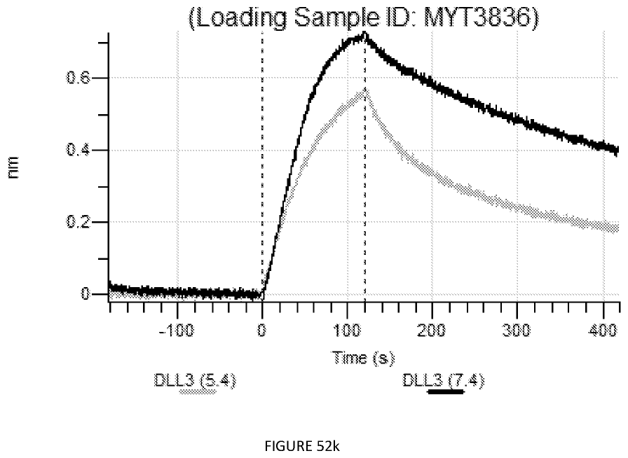

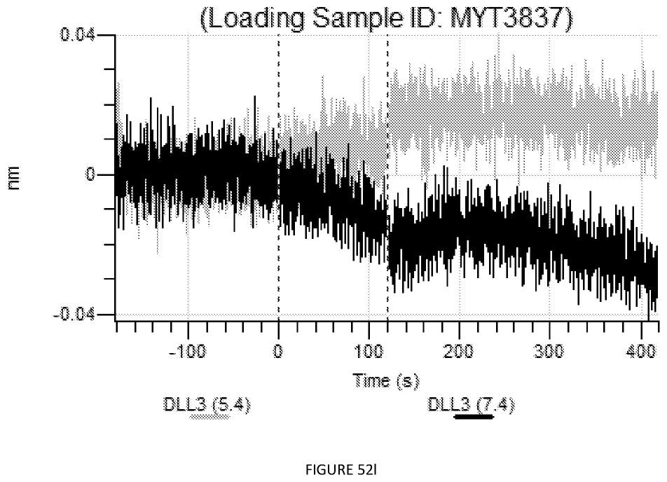

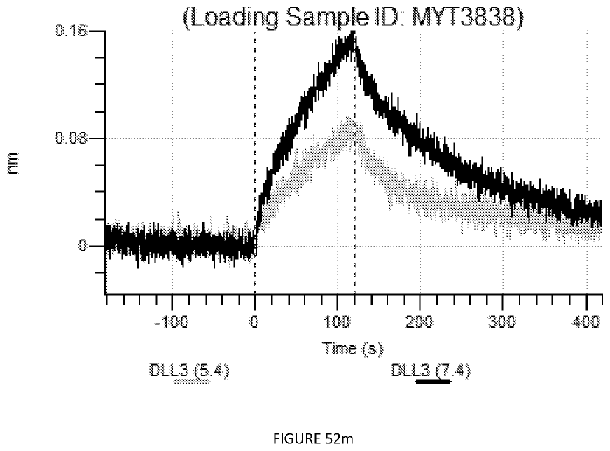

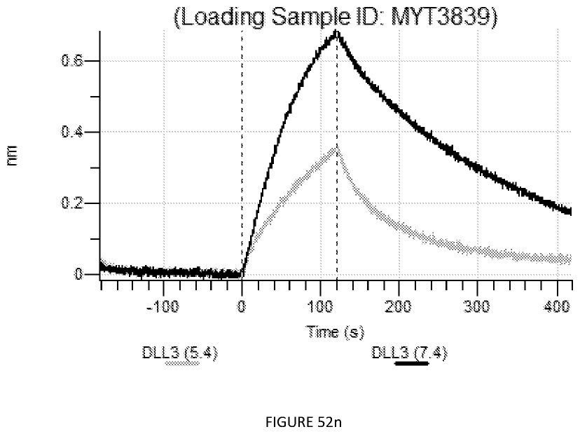

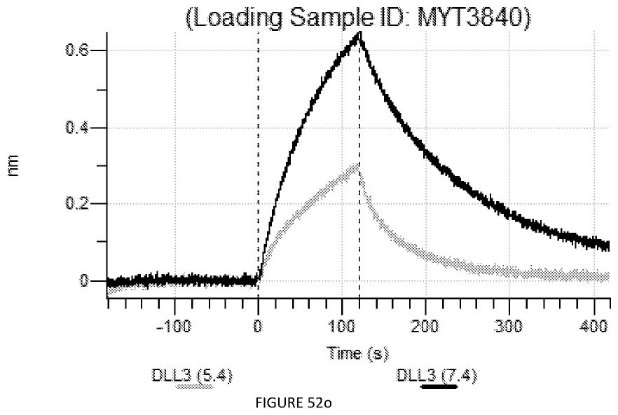

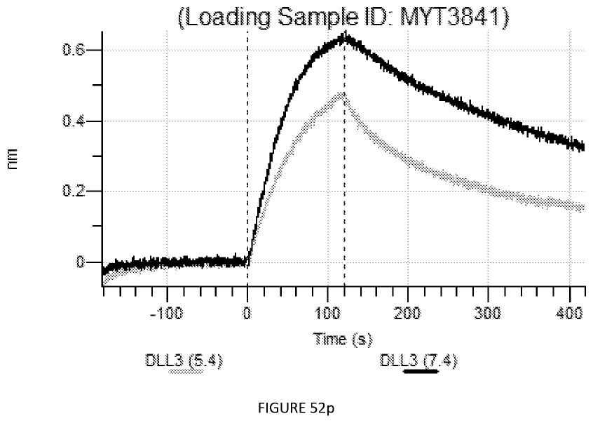

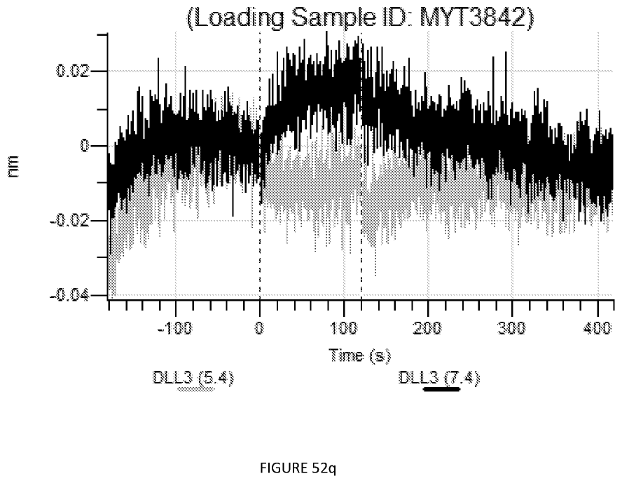

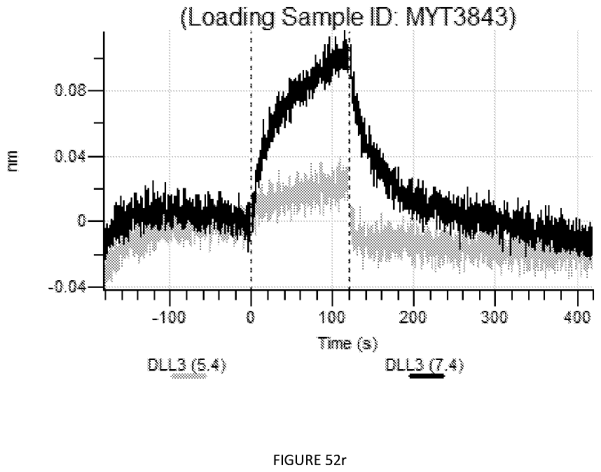

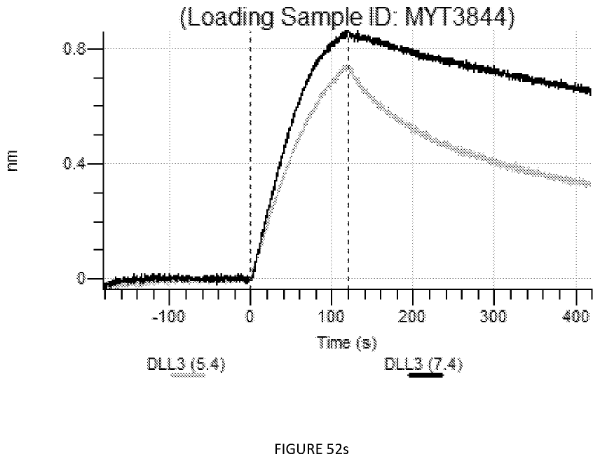

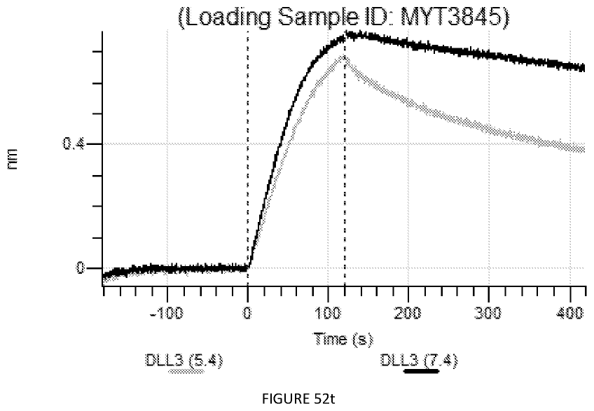

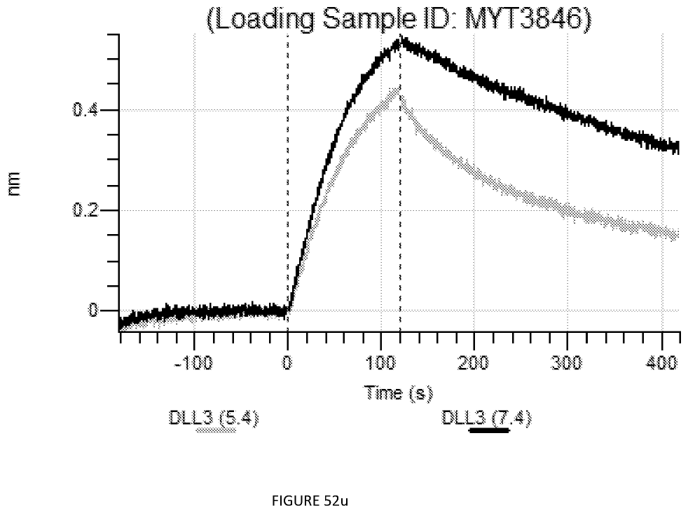

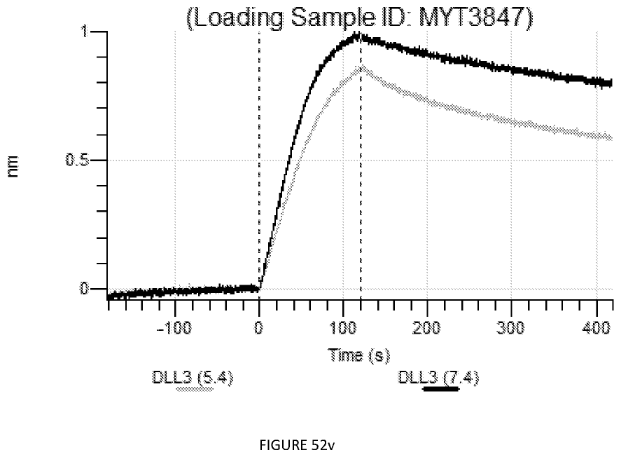

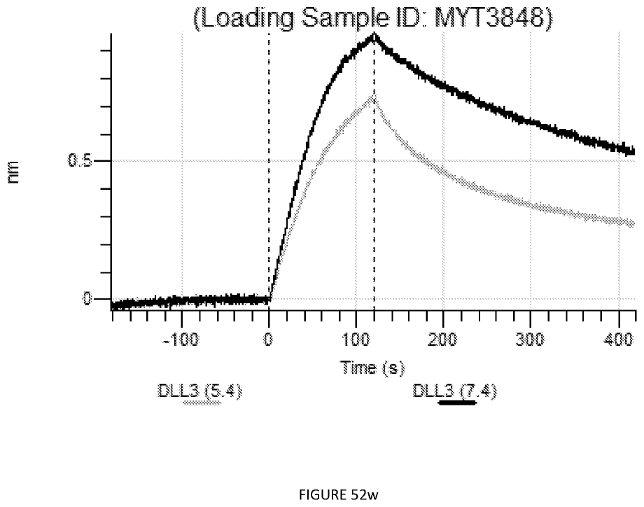

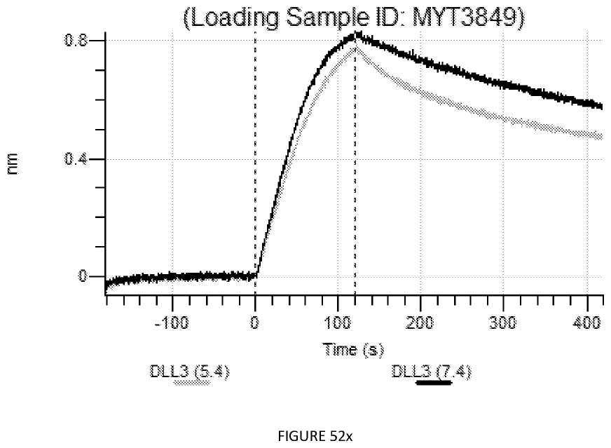

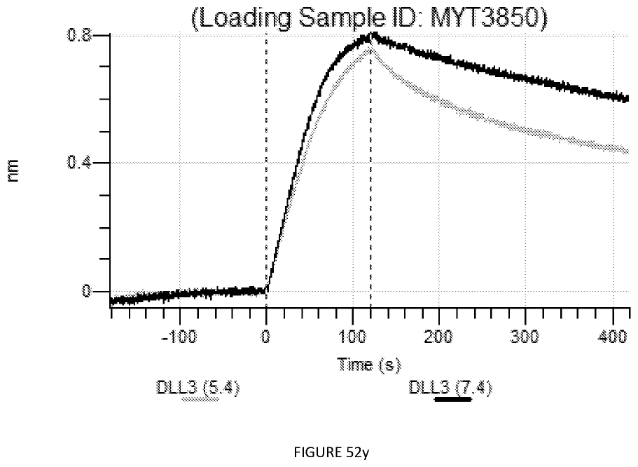

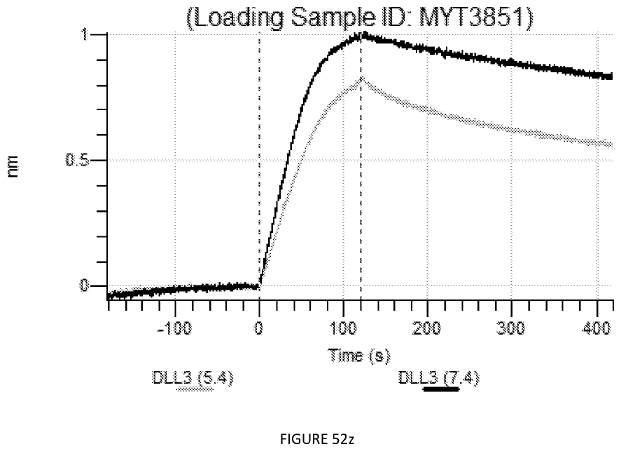

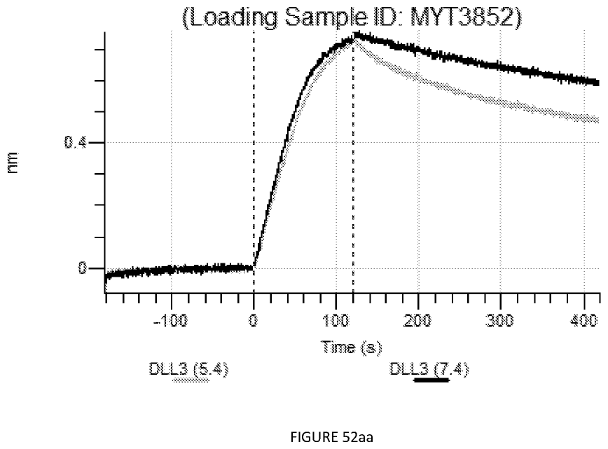

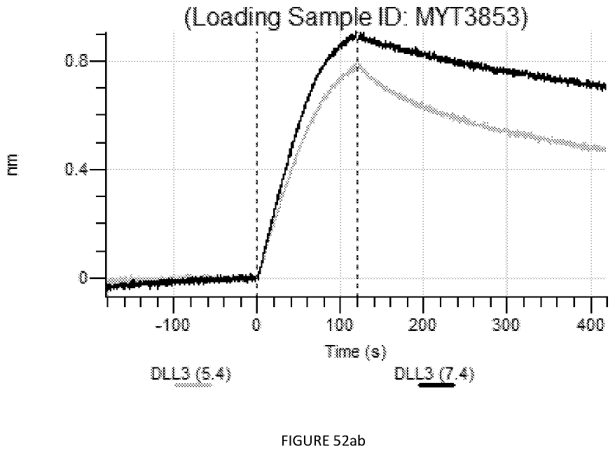

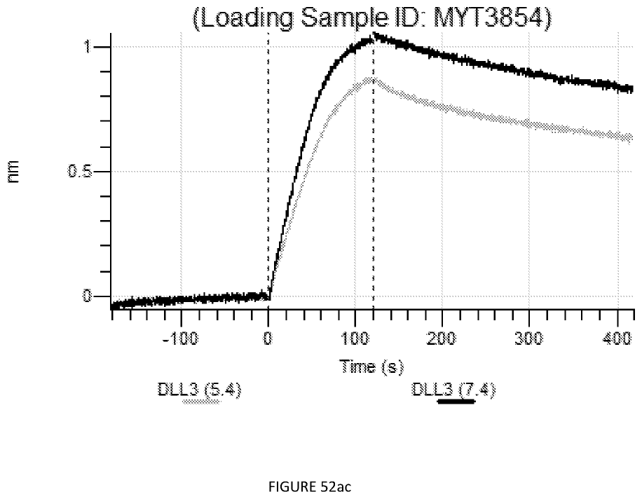

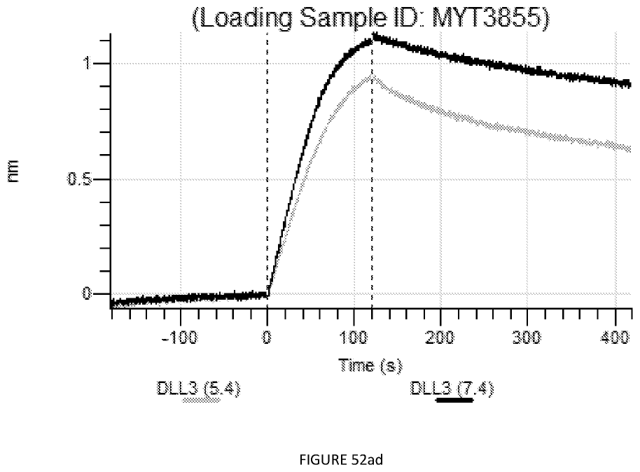

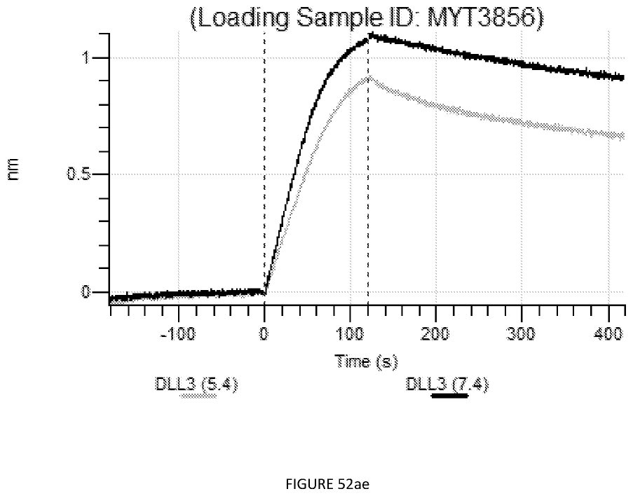

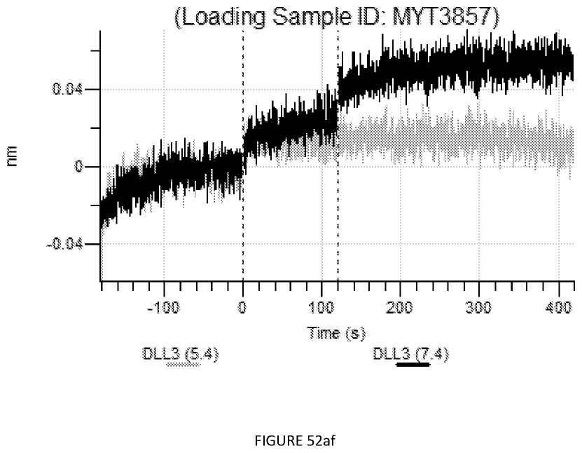

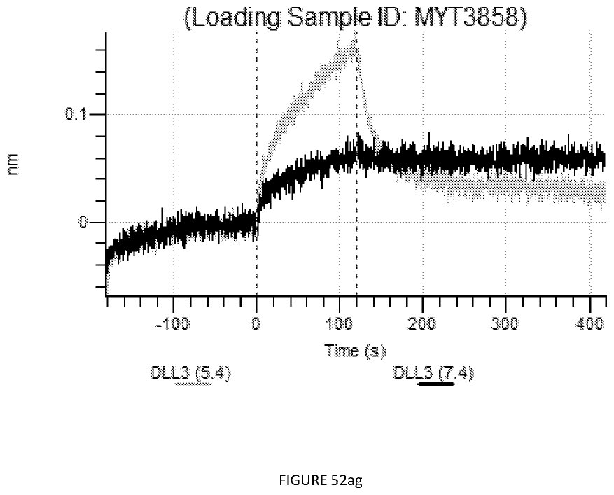

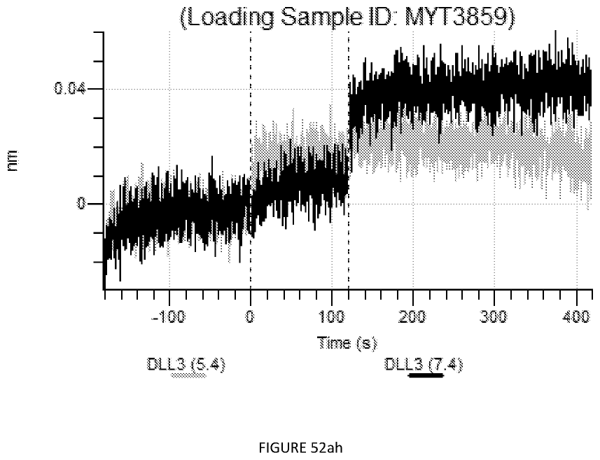

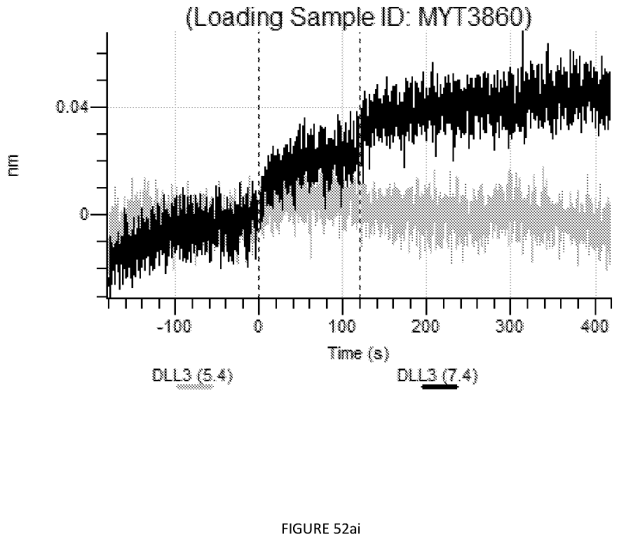

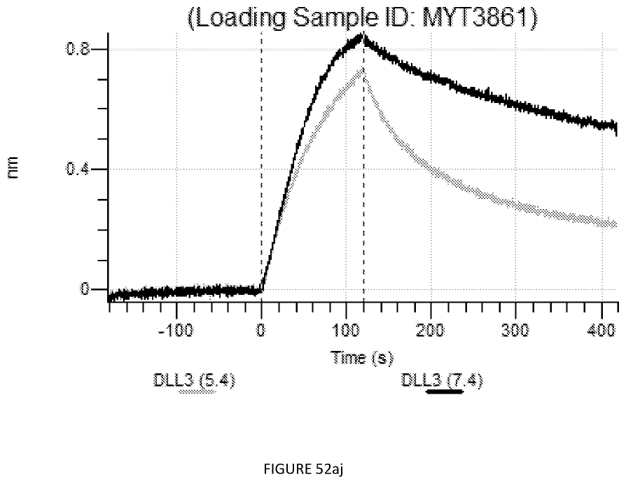

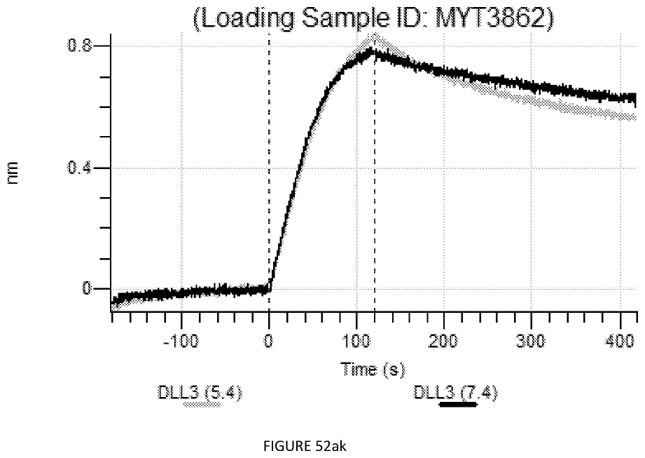

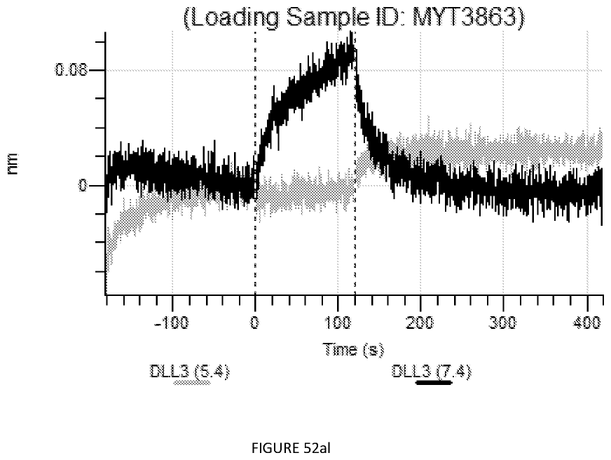

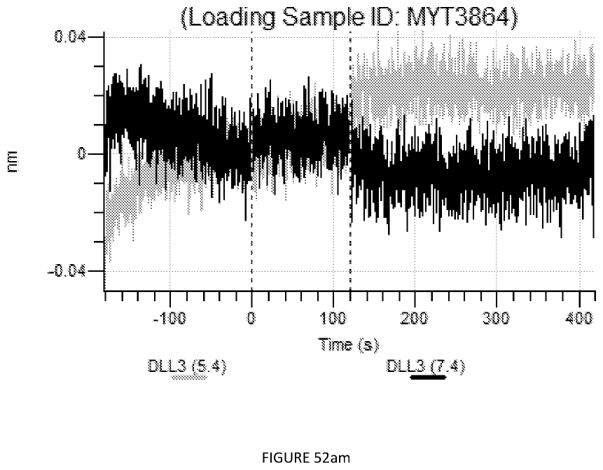

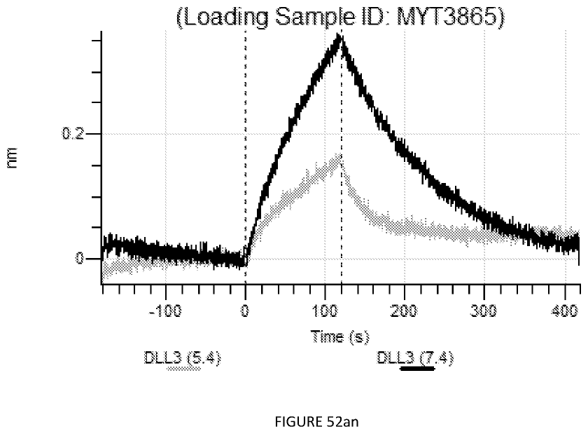

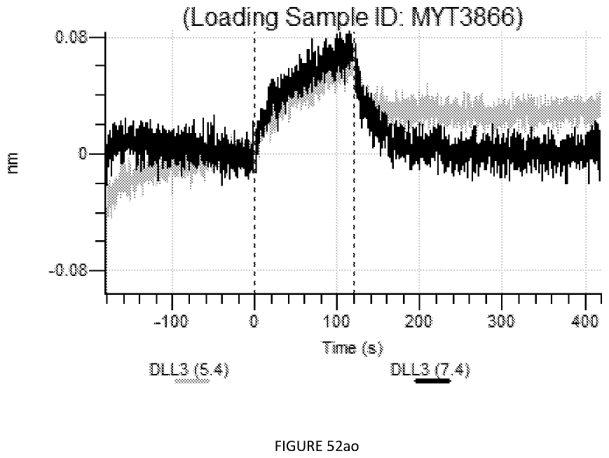

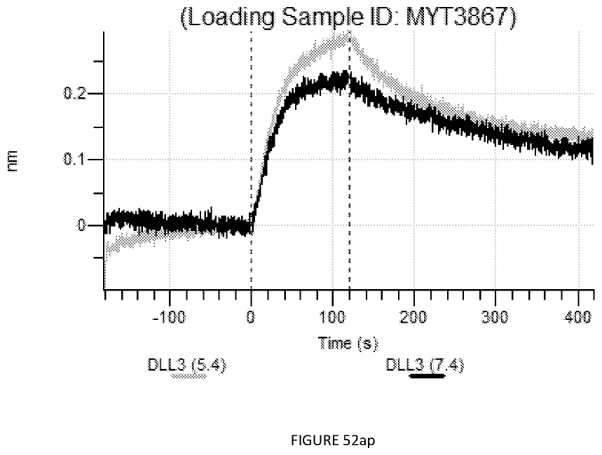

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.34 (U.S. Pat. No. 9,089,617 B2) (Heavy chain SEQ ID NO: 470, Light chain SEQ ID NO: 471) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3827-MYT3867). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 51) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 52, were selected for further analysis (MYT3828, MYT3831, MYT3833, MYT3836, MYT3838, MYT3839, MYT3840, MYT3841, MYT3843, MYT3844, MYT3846, MYT3848, MYT3849, MYT3850, MYT3853, MYT3858, MYT3861, MYT3863, MYT3865, MYT3866, and MYT3867). It was also noted that while some histidine and alanine mutations obliterated DLL3 binding (e.g., MYT3837, MYT3842, MYT3857, MYT3859, MYT3860, and MYT3864), others were tolerated with little (e.g., less than 1-fold change in dissociation constant KD or dissociation rate) or no change in DLL3 binding kinetics (e.g., MYT3827, MYT3829, MYT3830, MYT3832, MYT3834, MYT3835, MYT3845, MYT3847, MYT3851, MYT3852, MYT3854, MYT3855, MYT3856, MYT3862). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 29. Construction and Screening of pH-Engineered DLL3 ABPCs

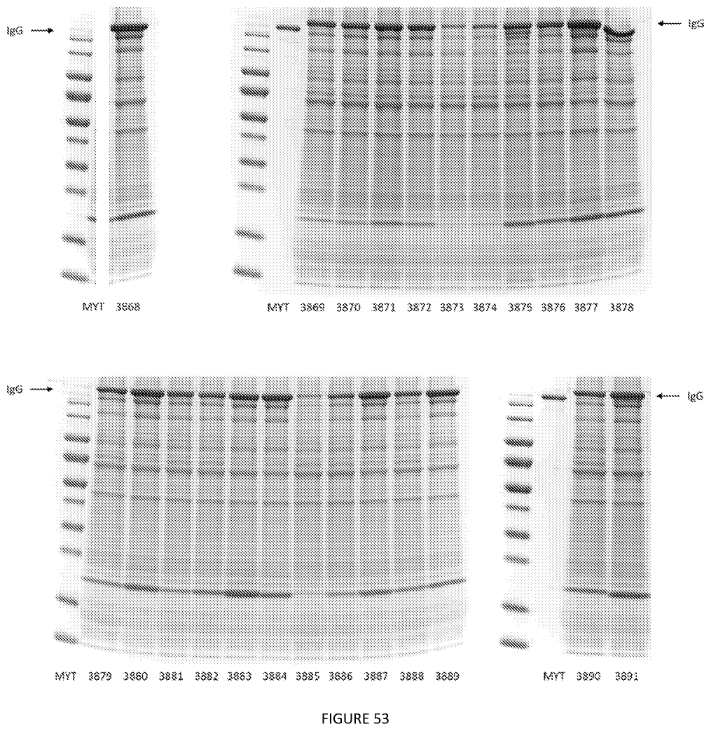

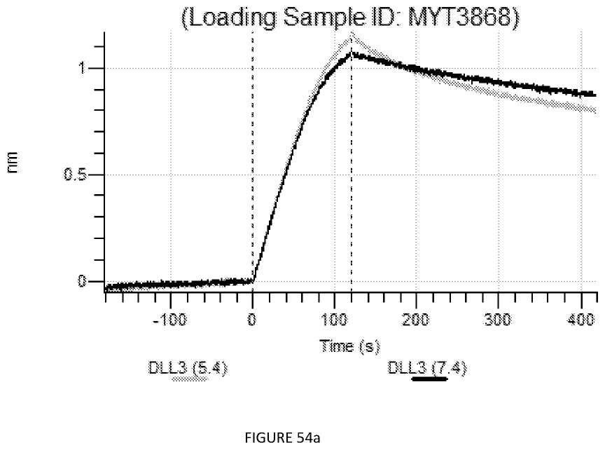

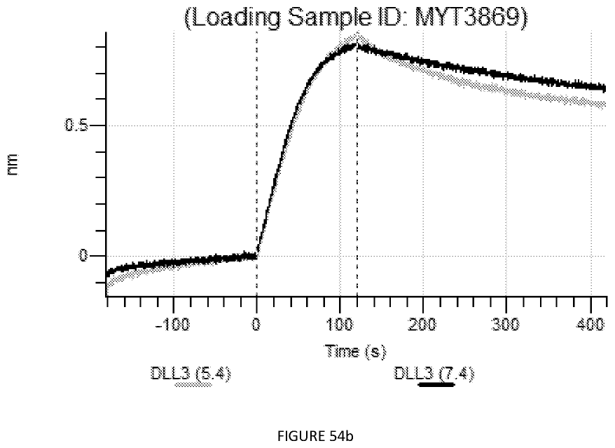

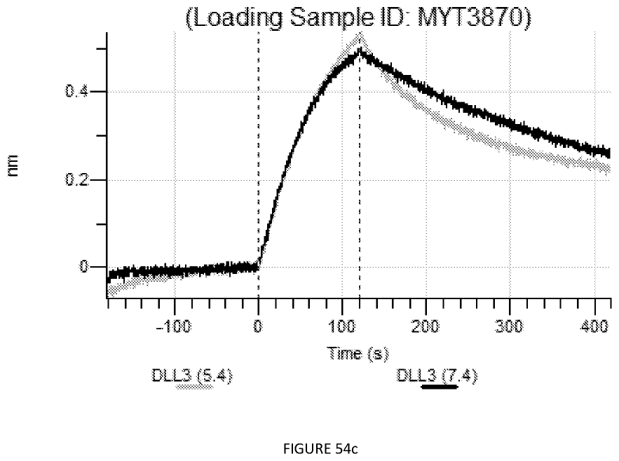

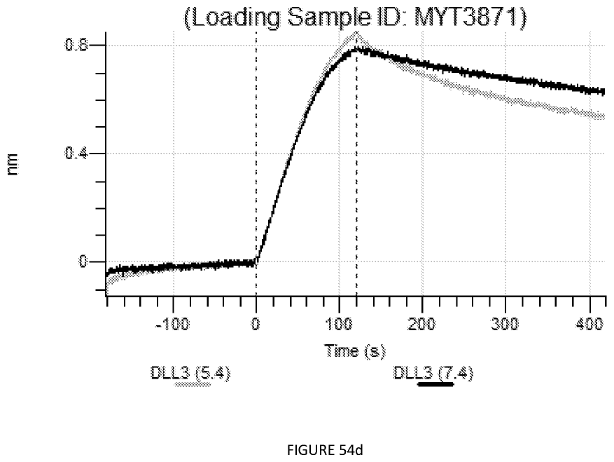

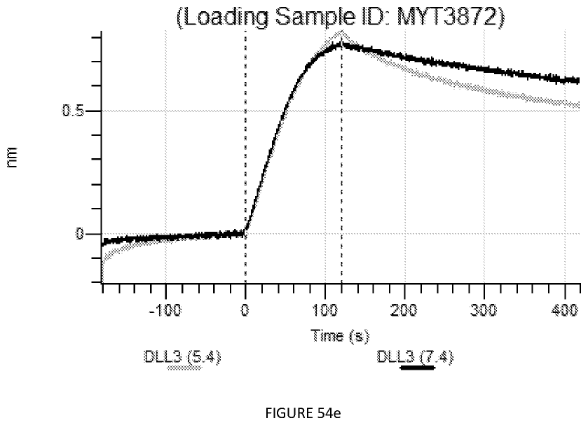

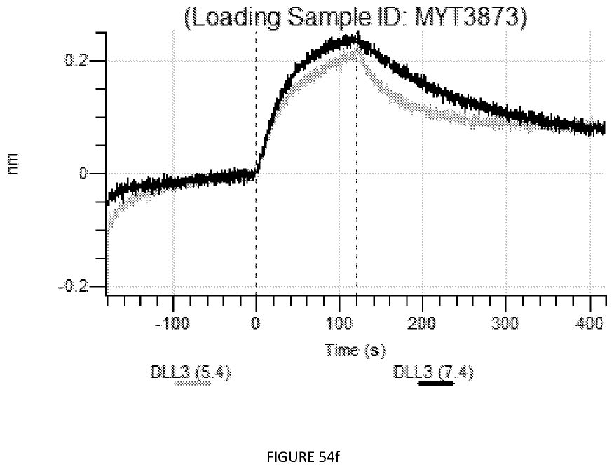

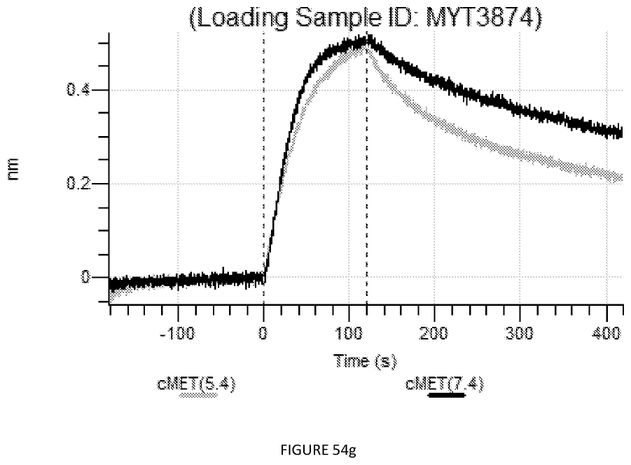

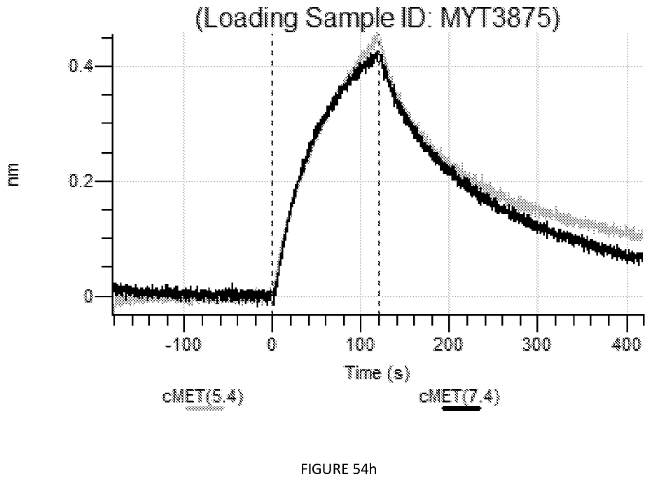

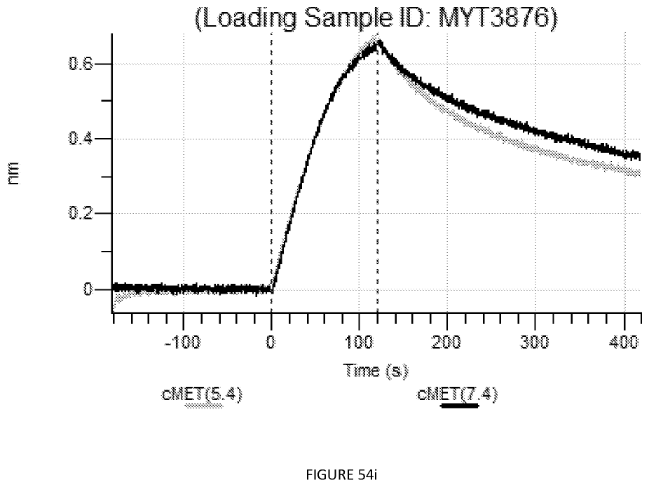

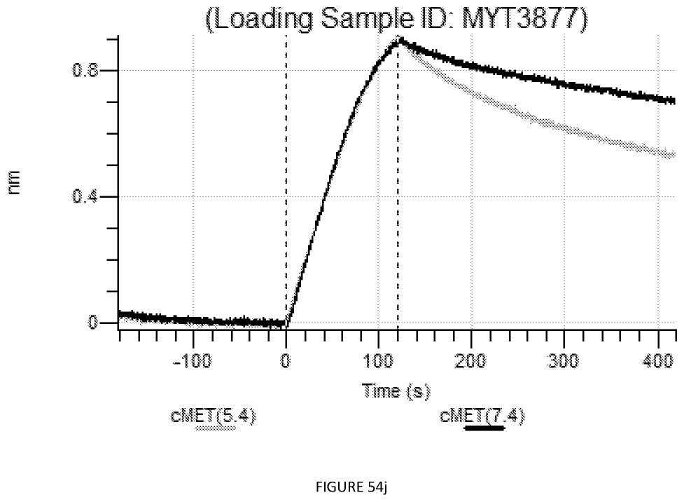

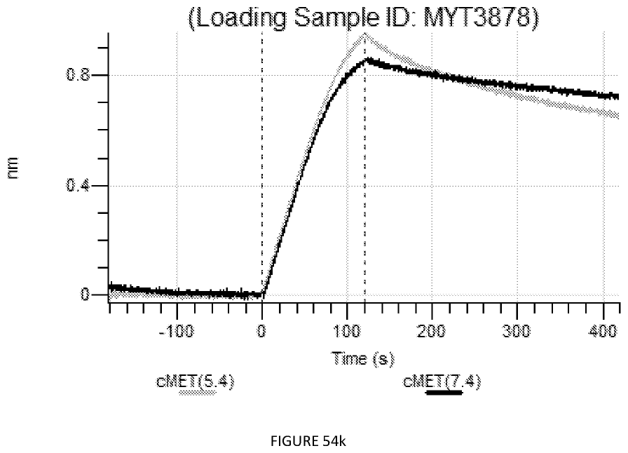

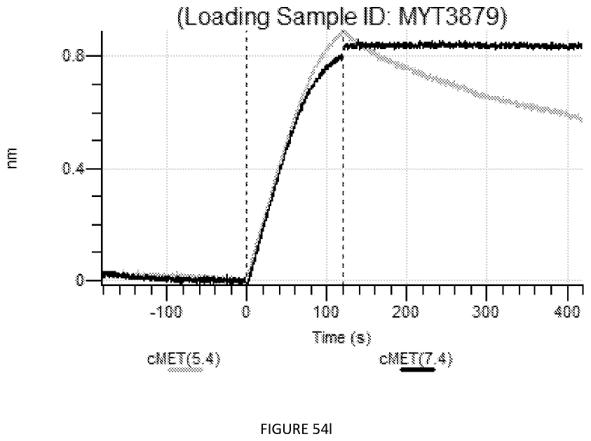

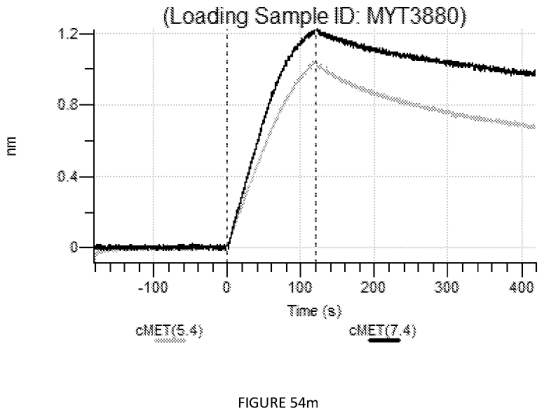

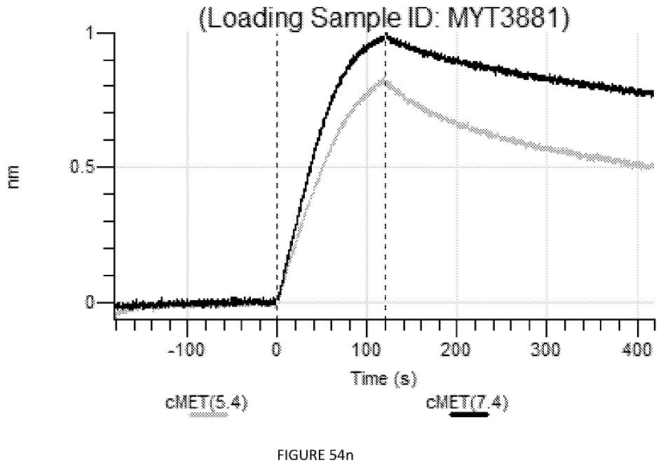

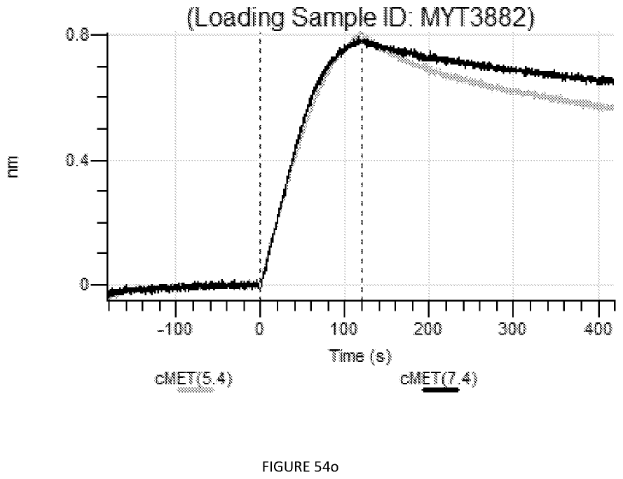

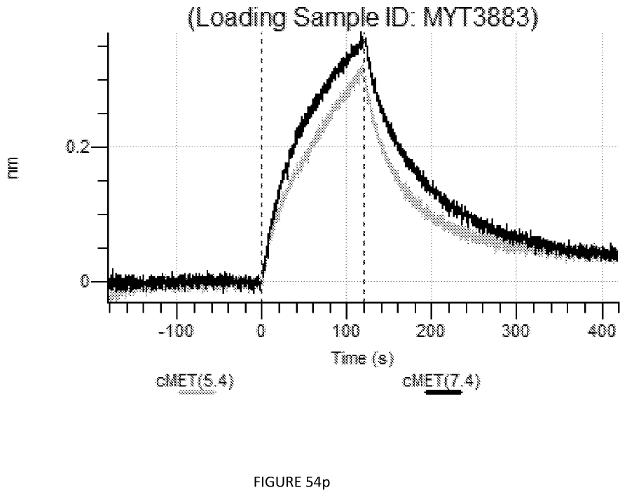

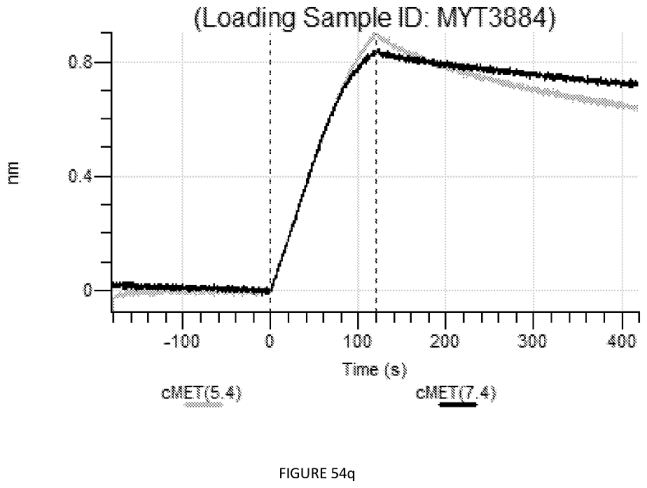

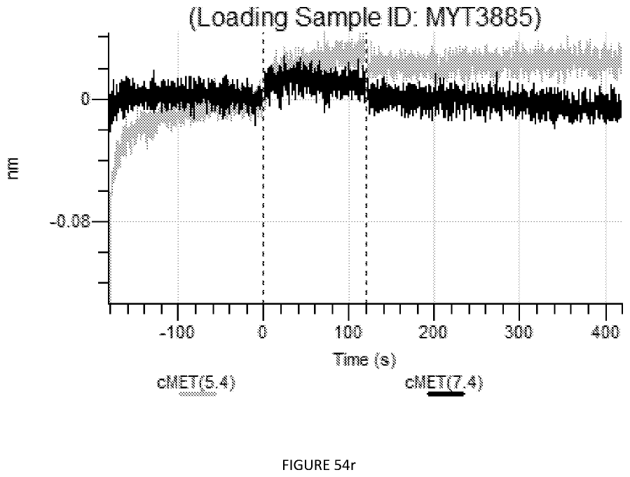

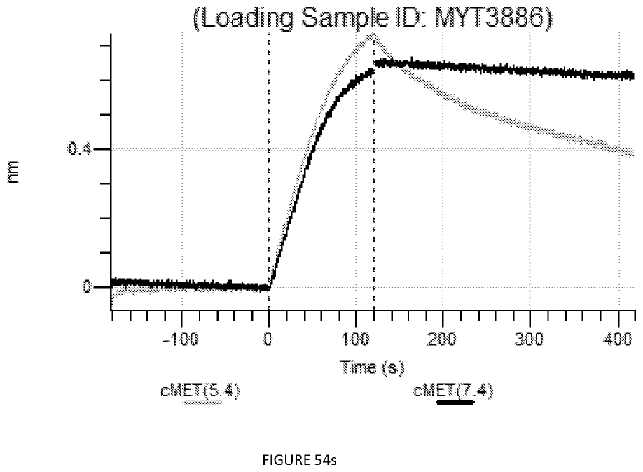

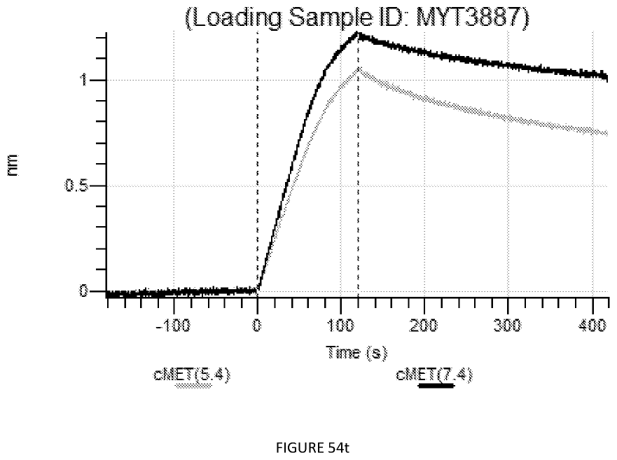

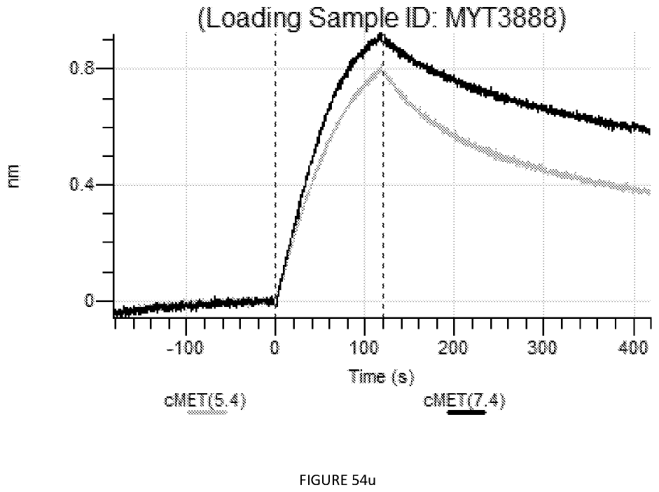

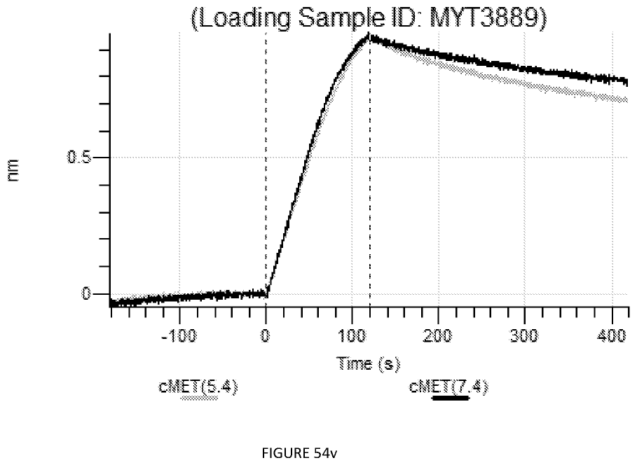

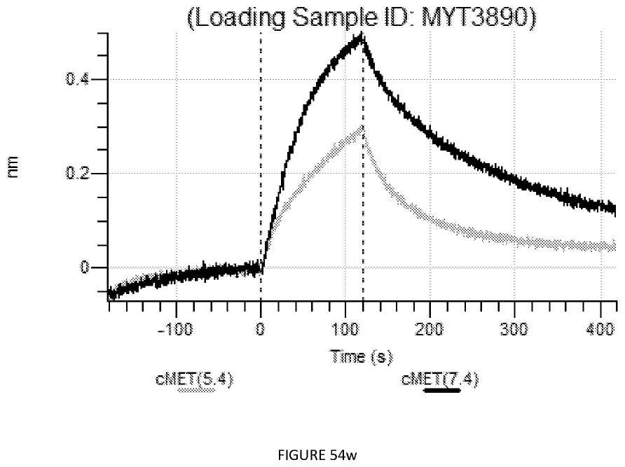

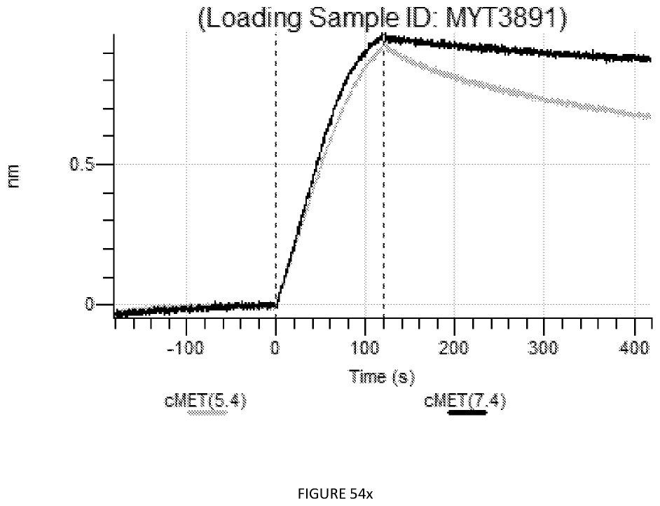

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). hSC16.34 (U.S. Pat. No. 9,089,617 B2) (Heavy chain SEQ ID NO: 470, Light chain SEQ ID NO: 471) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3868-MYT3891). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 53), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC), as shown in FIG. 54, were selected for further analysis (e.g., MYT3870, MYT3873-MYT3877, MYT3879, MYT3881, MYT3883, MYT3886, MYT3888, MYT3890, and MYT3891). It was also noted that while some histidine and alanine mutations obliterated DLL3 binding (e.g., MYT3885), others were tolerated with little (e.g., less than 1-fold change in dissociation constant KD or dissociation rate) or no change in DLL3 binding kinetics (e.g., MYT3868, MYT3869, MYT3871, MYT3872, MYT3878, MYT3880, MYT3882, MYT3884, MYT3887, and MYT3889). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 30. Construction and Screening of pH-Engineered DLL3 ABPCs

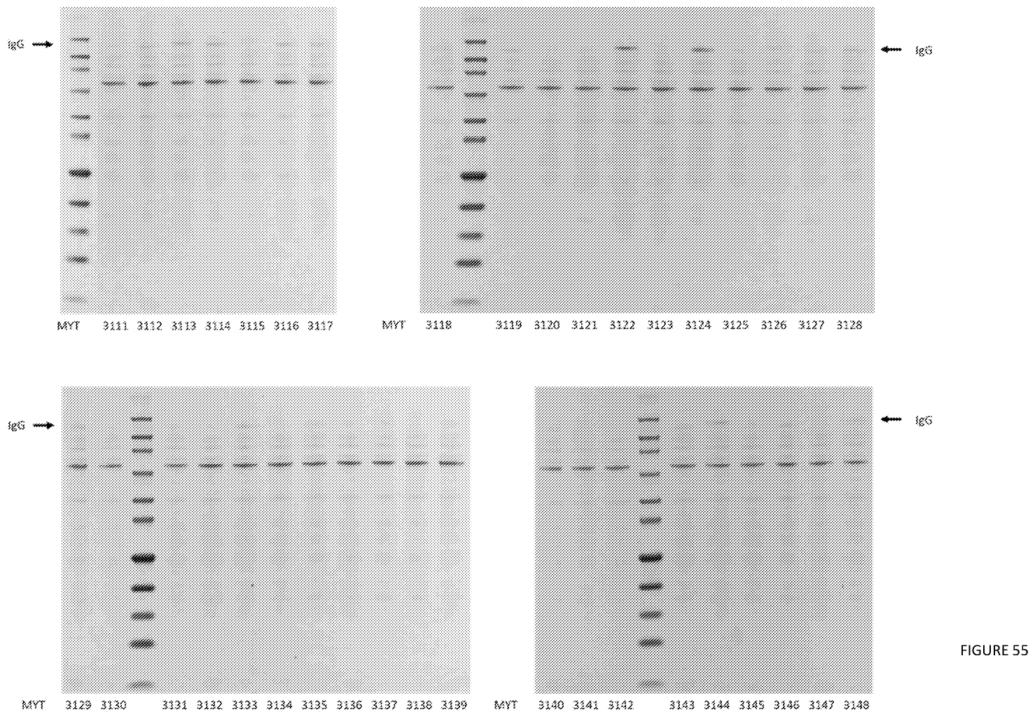

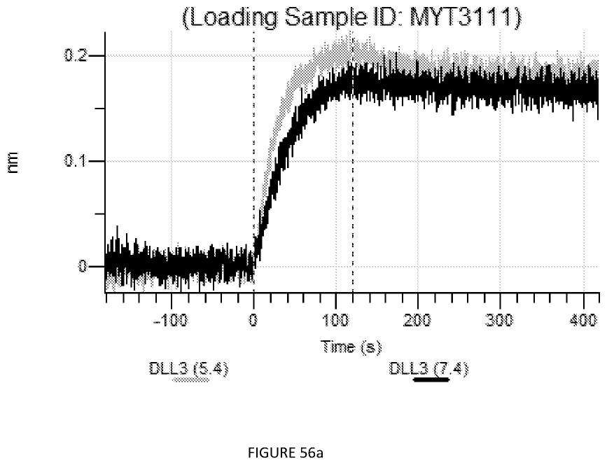

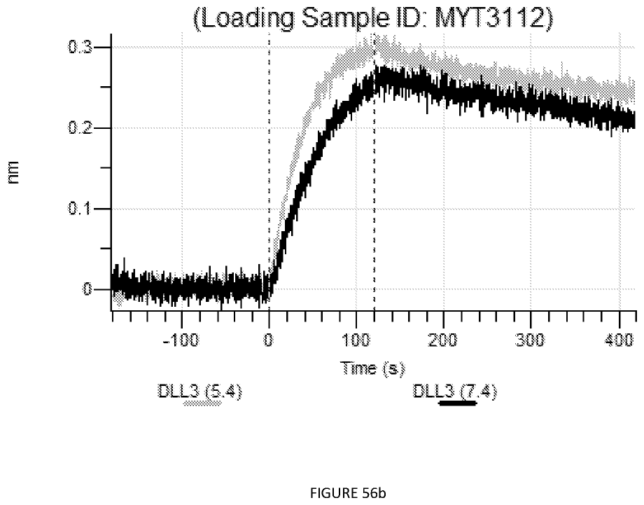

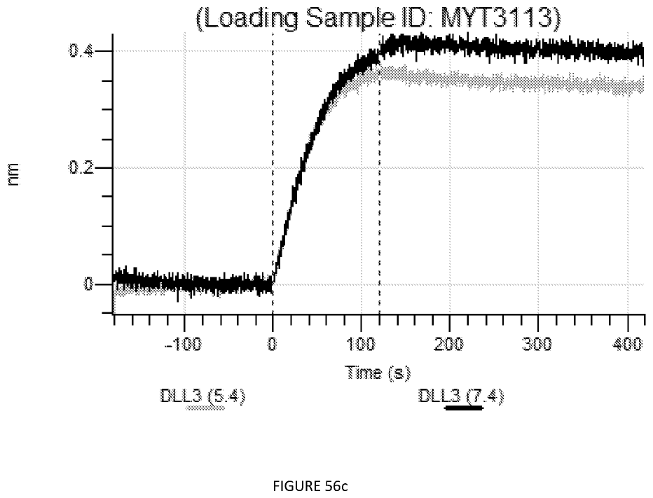

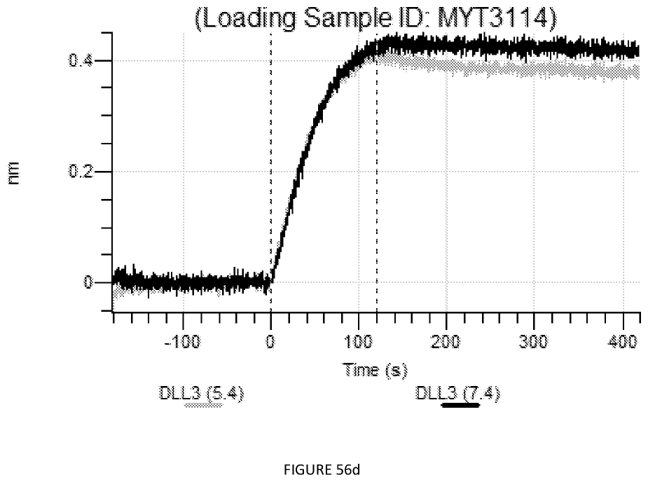

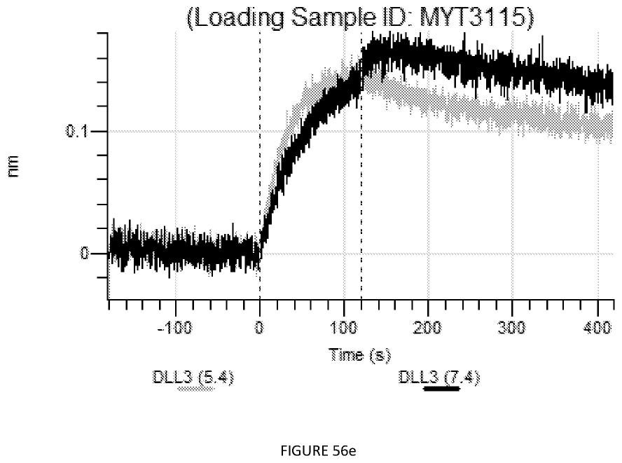

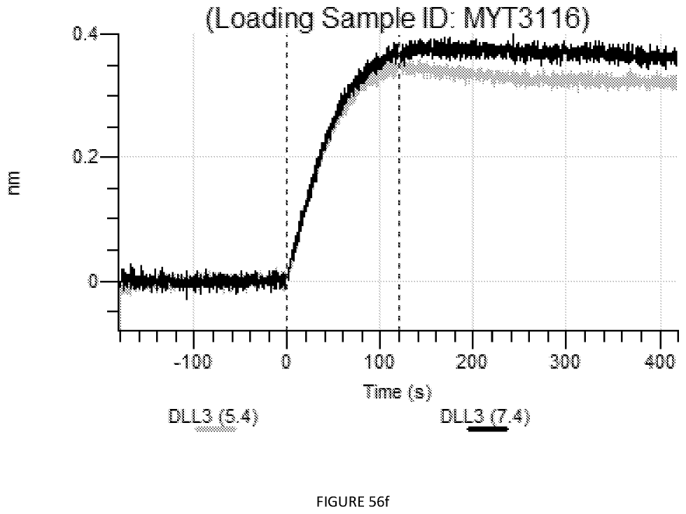

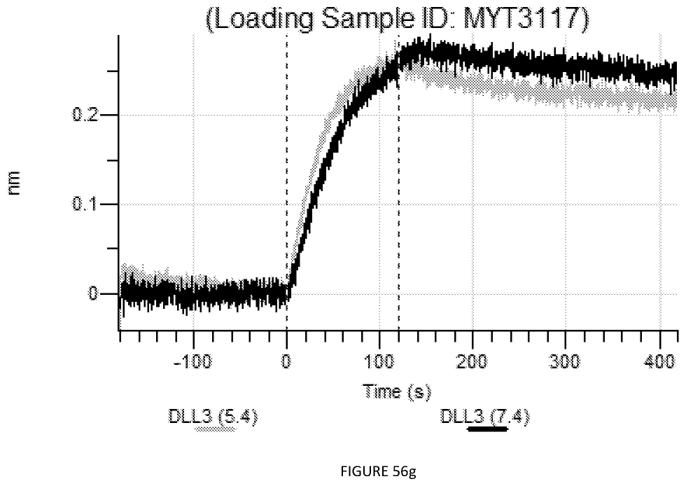

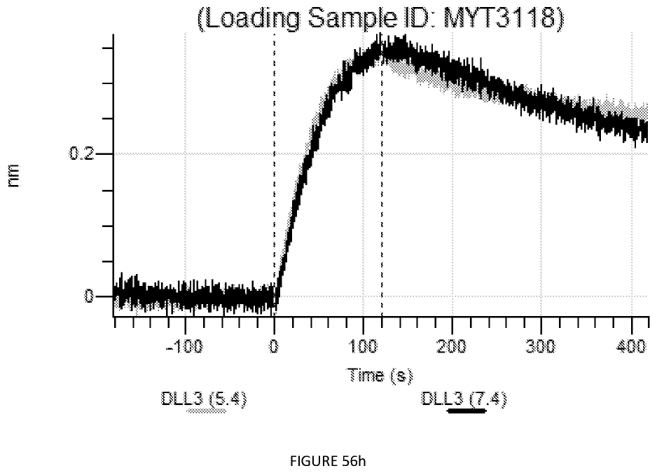

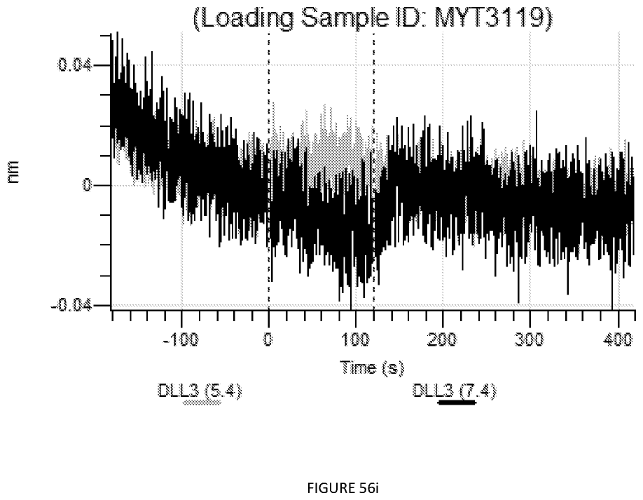

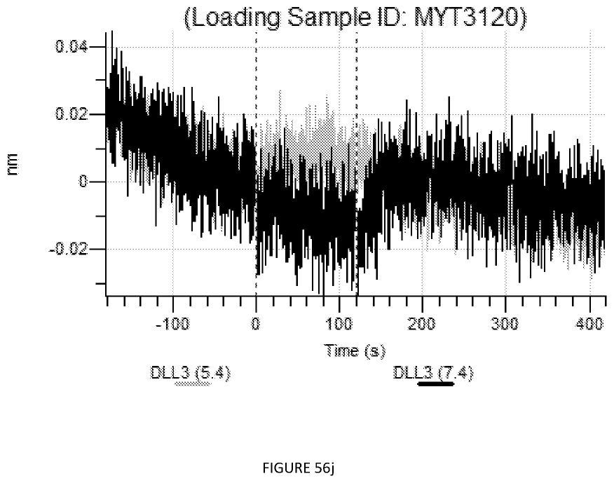

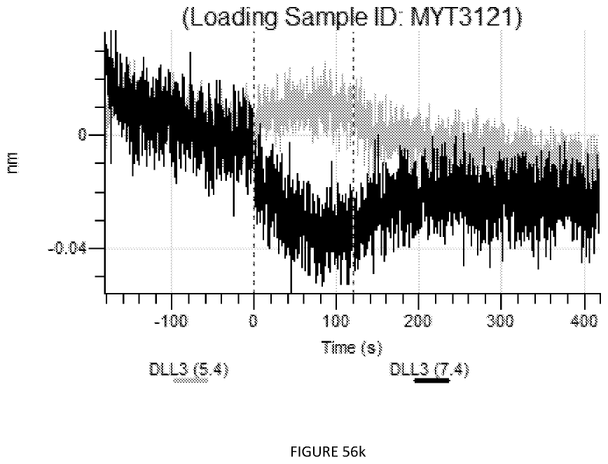

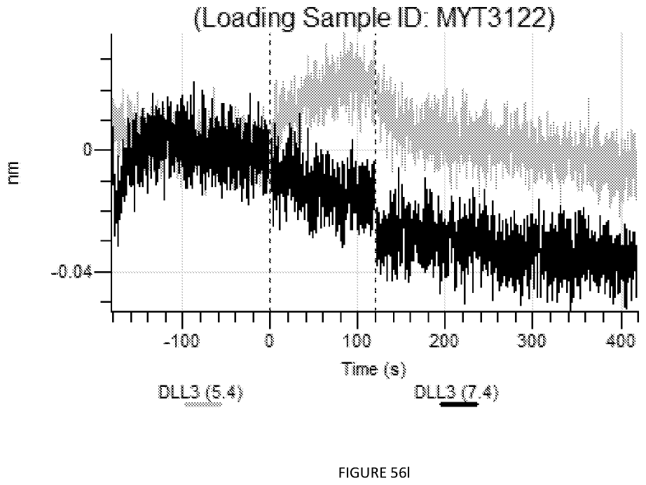

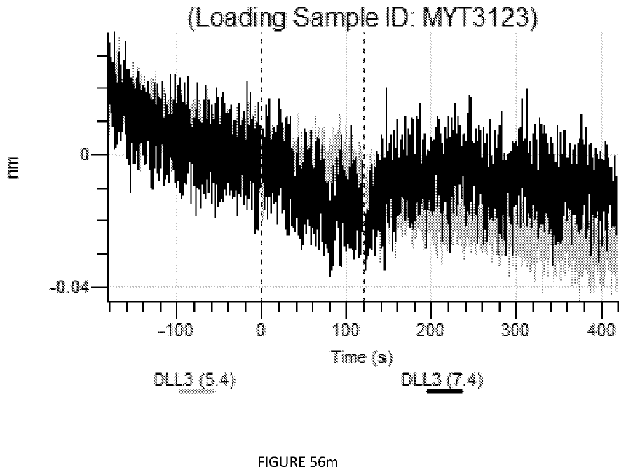

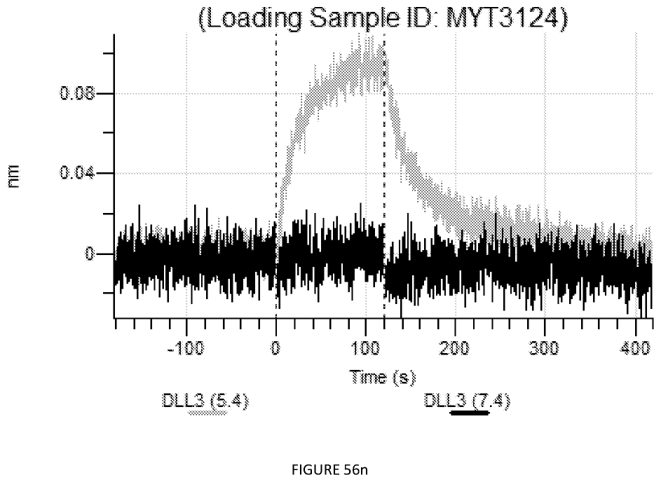

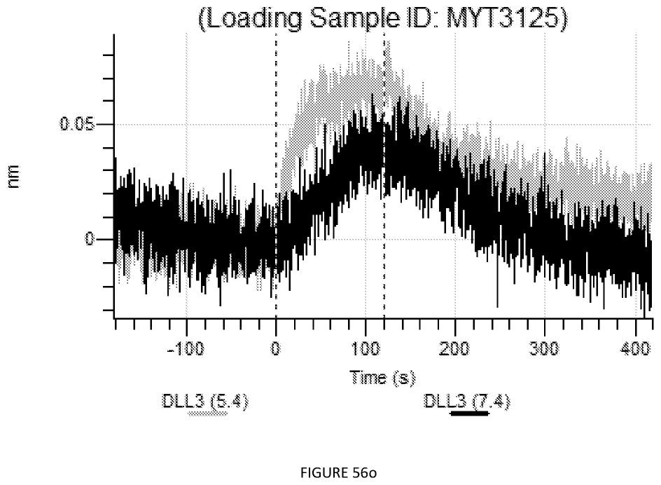

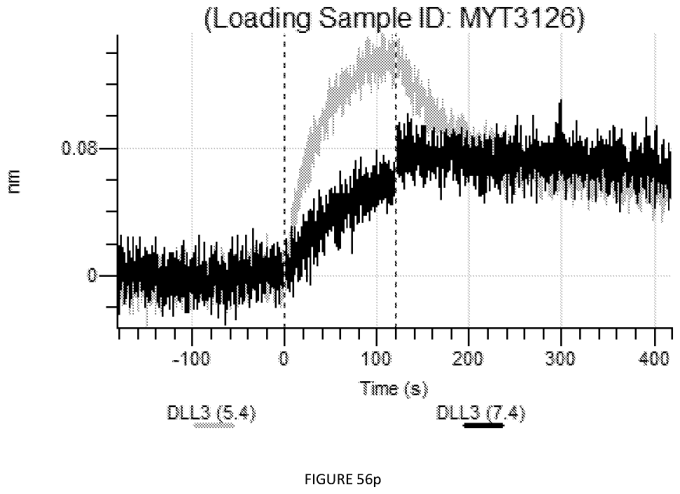

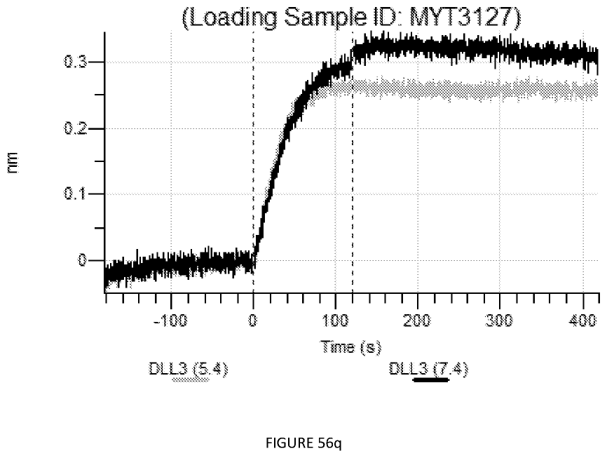

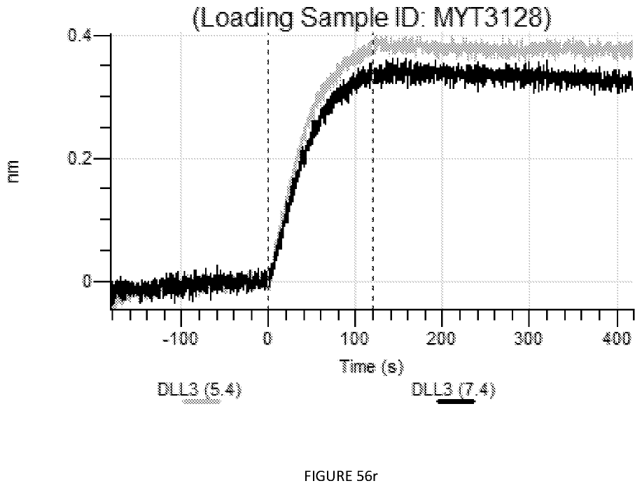

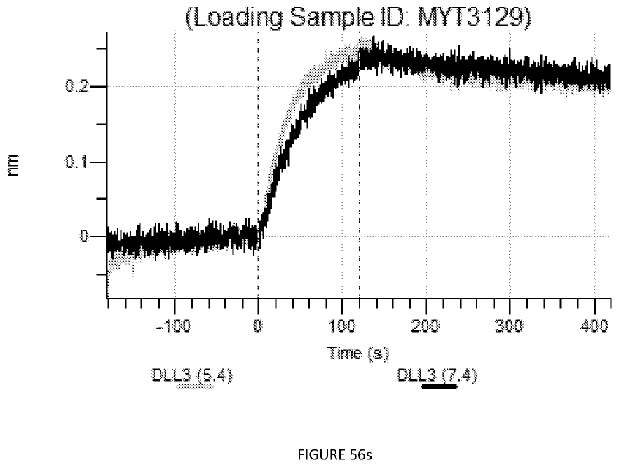

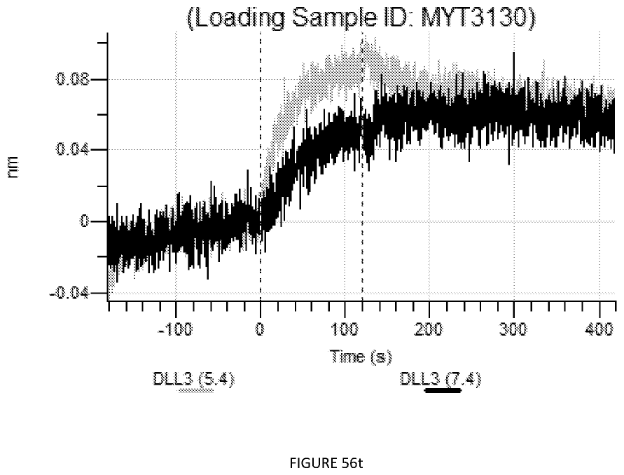

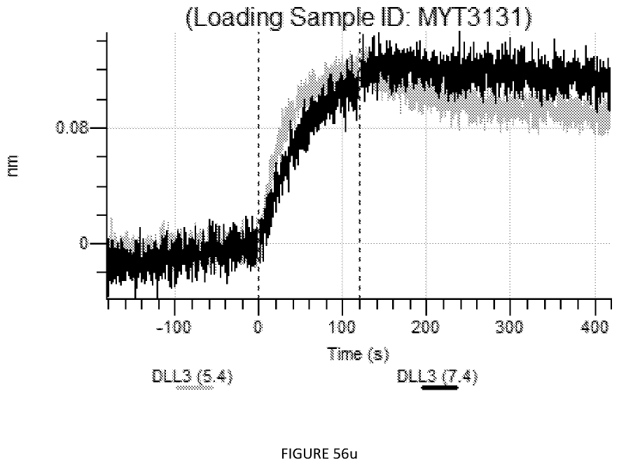

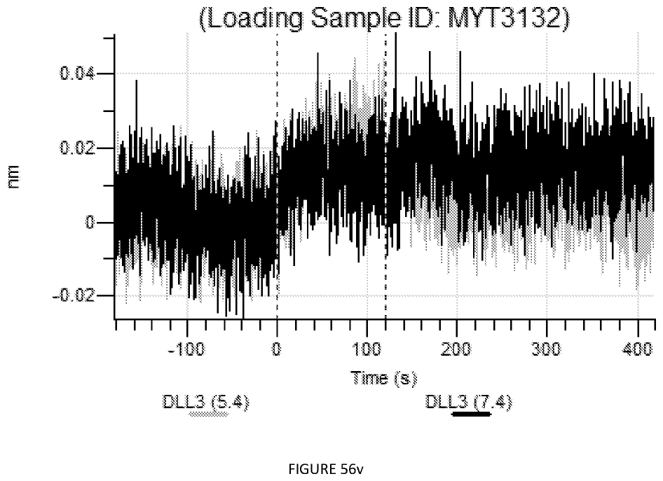

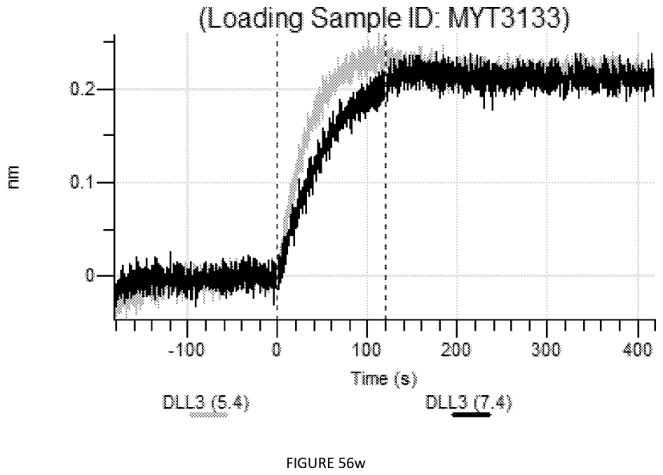

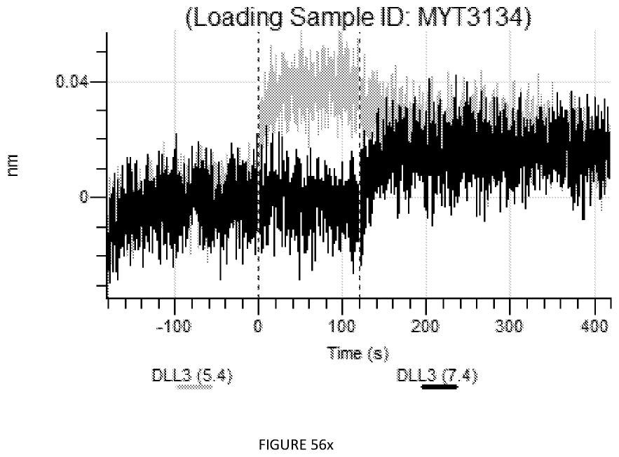

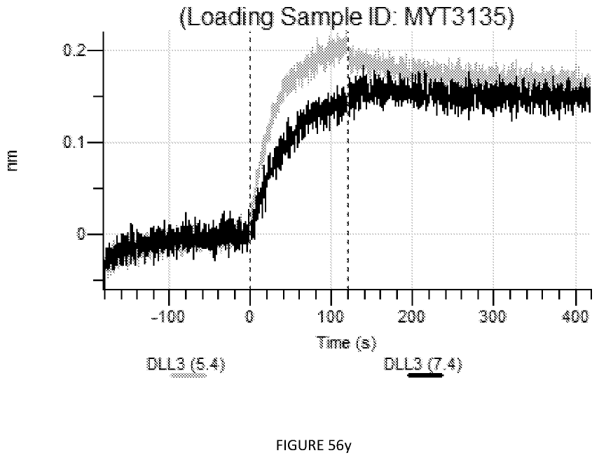

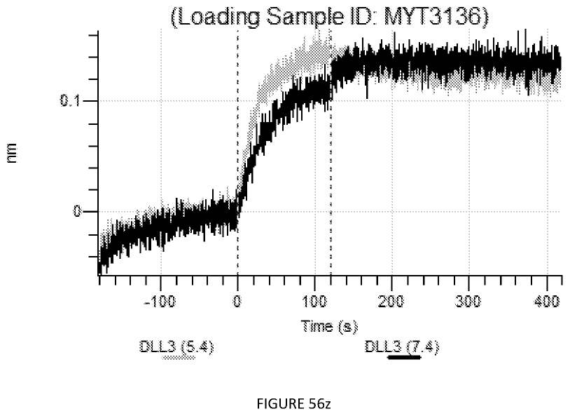

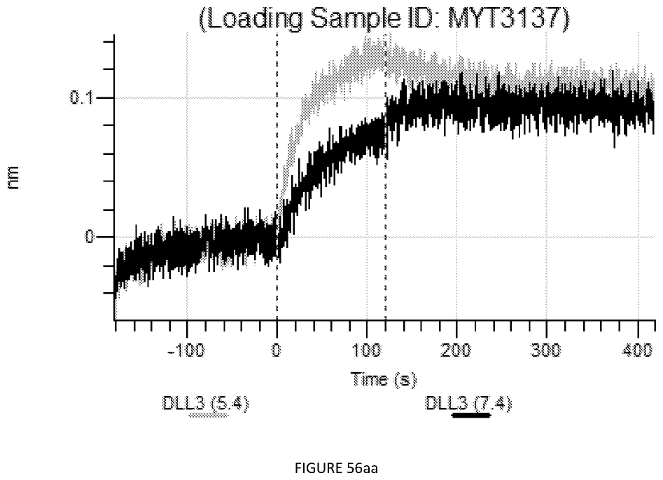

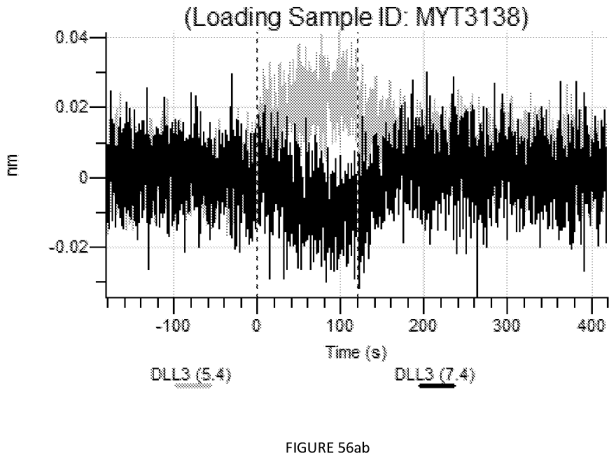

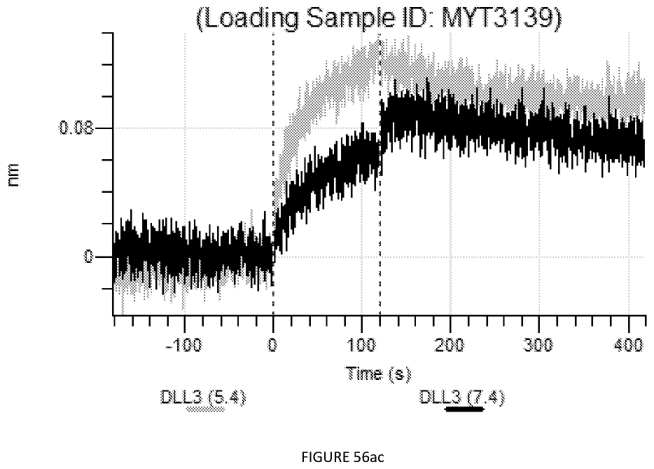

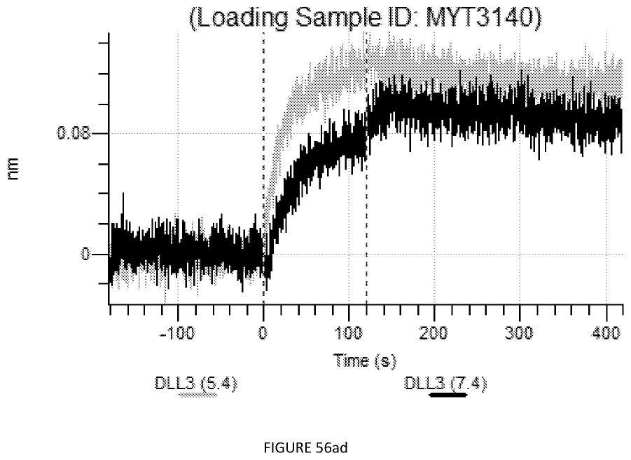

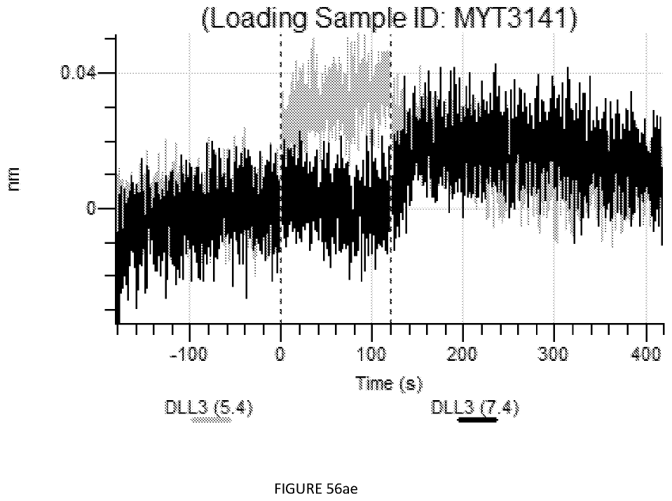

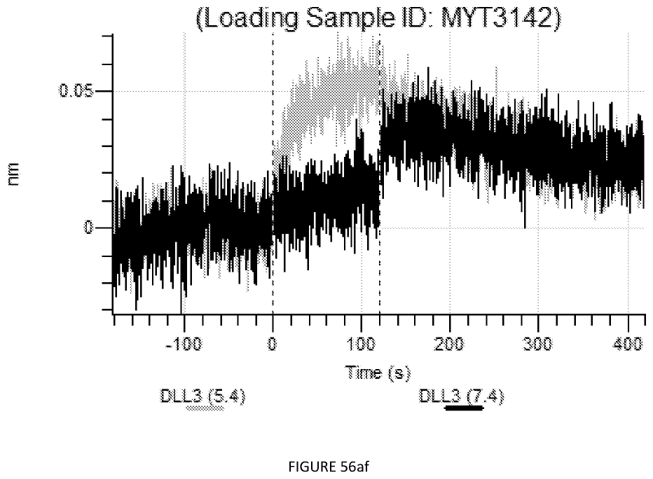

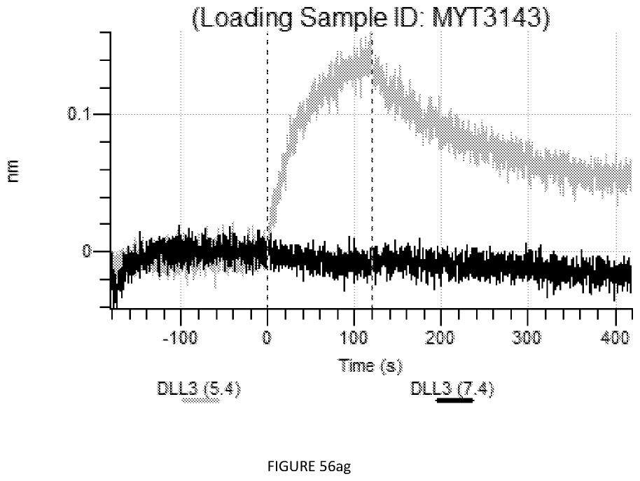

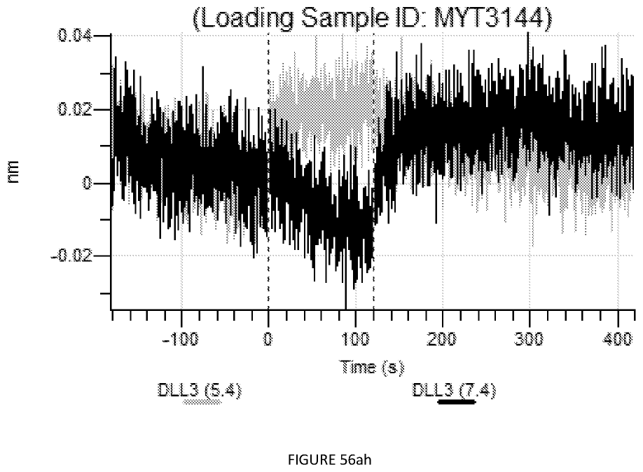

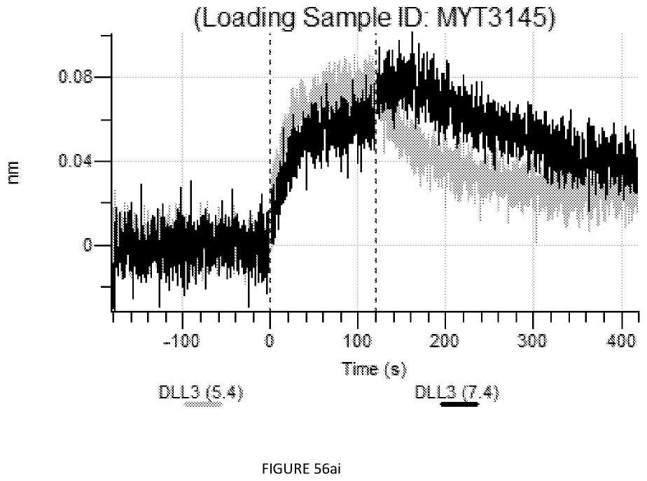

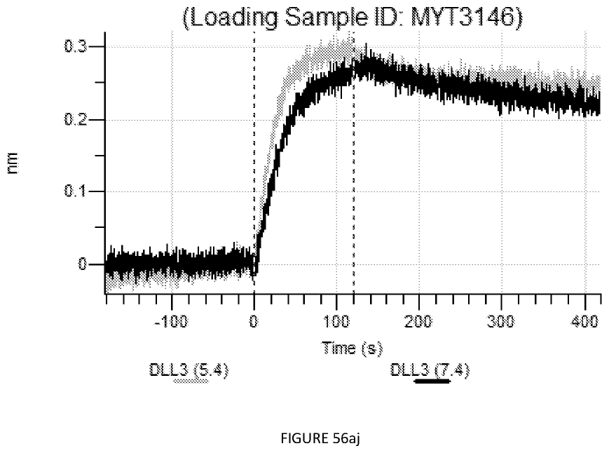

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.67 (U.S. Pat. No. 9,089,617 B2) (Heavy chain SEQ ID NO: 537, Light chain SEQ ID NO: 538) was selected as a DLL3-binding monoclonal antibody for pH engineering via histidine scanning. Briefly, CDRs in the heavy chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the heavy chain CDRs were systematically substituted with a histidine, one at a time (MYT3112-MYT3148). In cases where the starting CDR residue was a histidine, it was mutated to an alanine. Antibody variants with only one histidine or alanine mutation in a heavy chain CDR were generated by co-transfection of Expi293 cells with a) one heavy chain sequence variant, and b) the corresponding starting ABPC light chain using methods known to the art. After allowing for four days of protein expression, cell culture supernatants were collected, quantified by SDS-PAGE (FIG. 55) analysis, and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Heavy chain variants that showed either enhanced dissociation at pH 5.4 or reduced dissociation at pH 7.4 or both (as compared to the starting ABPC), as shown in FIG. 56, were selected for further analysis (MYT3112, MYT3115, MYT3117, MYT3118, MYT3122, MYT3124, MYT3125, MYT3126, MYT3129, MYT3130, MYT3131, MYT3134, MYT3137, MYT3139, MYT3140, MYT3143, MYT3145, MYT3146, and MYT3148). It was also noted that while some histidine and alanine mutations obliterated DLL3 binding (e.g., MYT3119, and MYT3144), others were tolerated with little (e.g., less than 1-fold change in dissociation constant KD or dissociation rate) or no change in DLL3 binding kinetics (e.g., MYT3113, MYT3114, MYT3116, MYT3127, MYT3128, MYT3133, MYT3135, and MYT3136). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 31. Construction and Screening of pH-Engineered DLL3 ABPCs

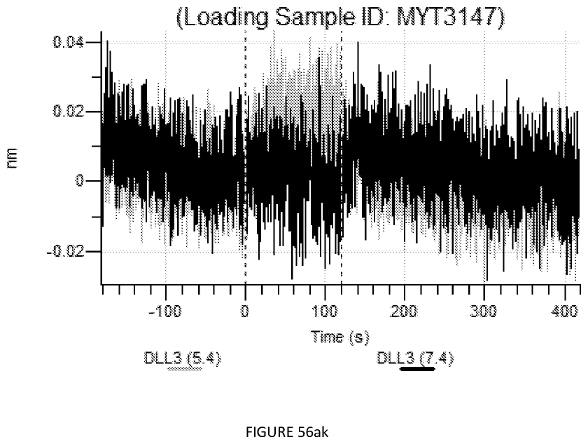

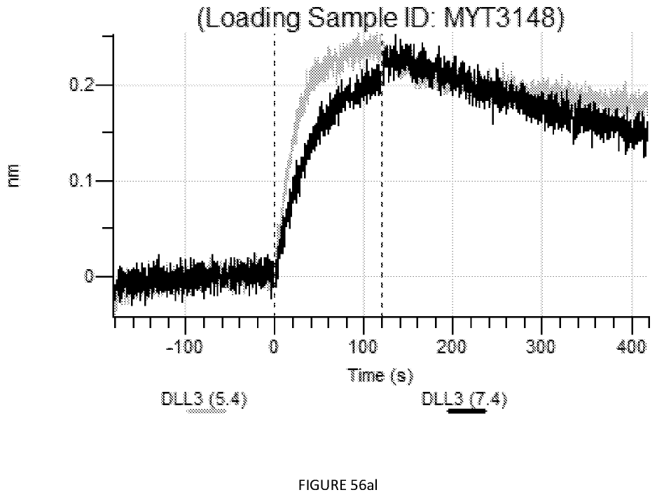

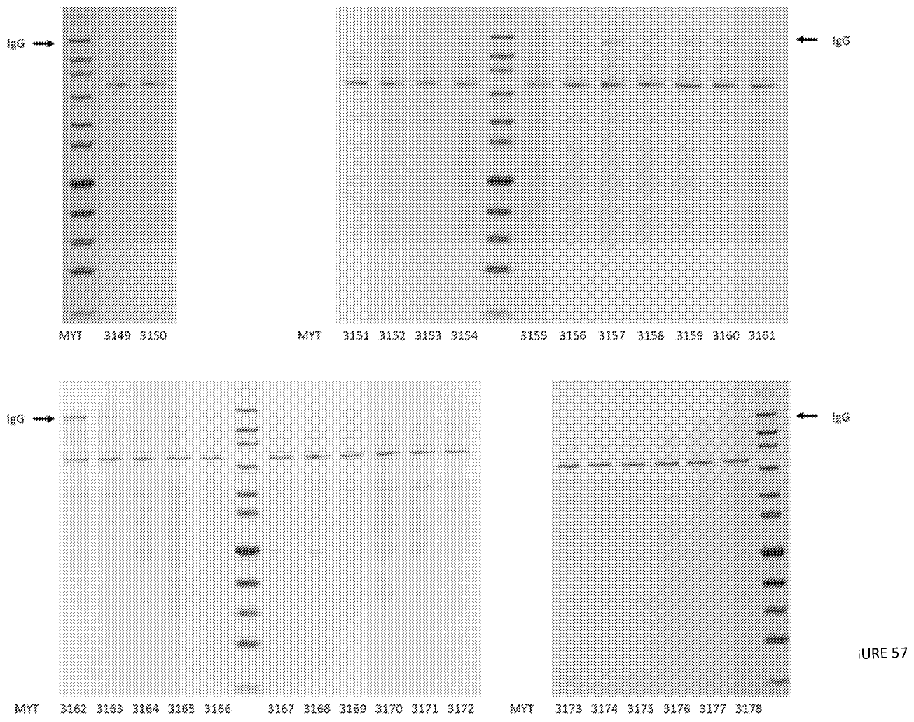

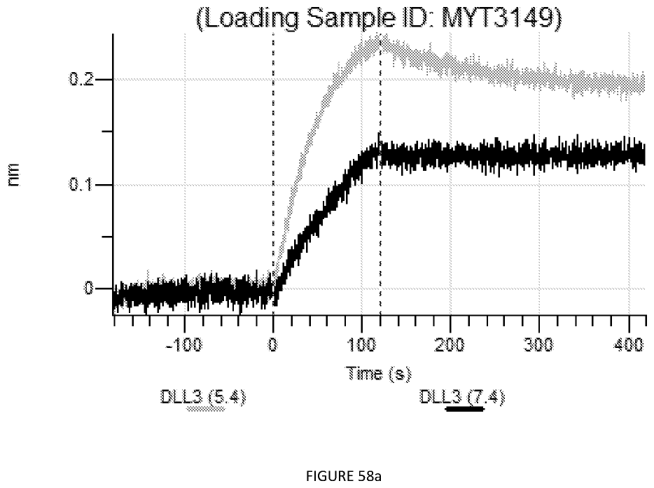

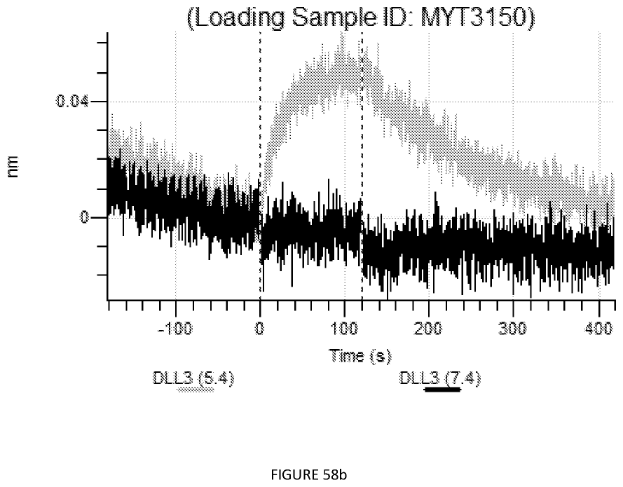

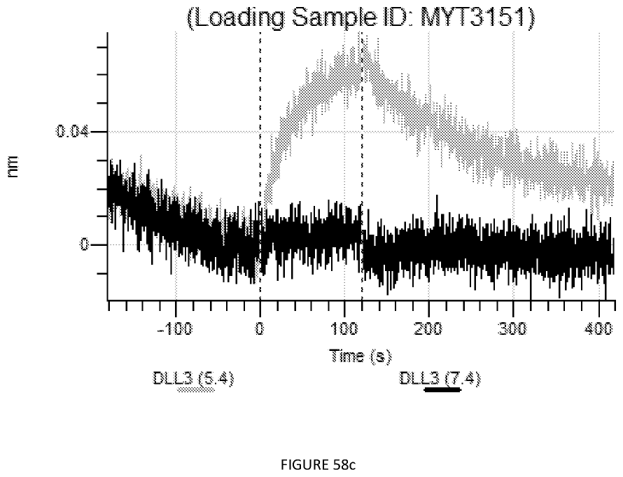

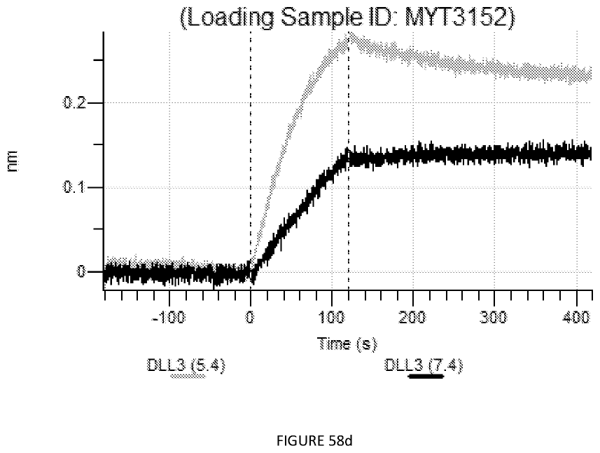

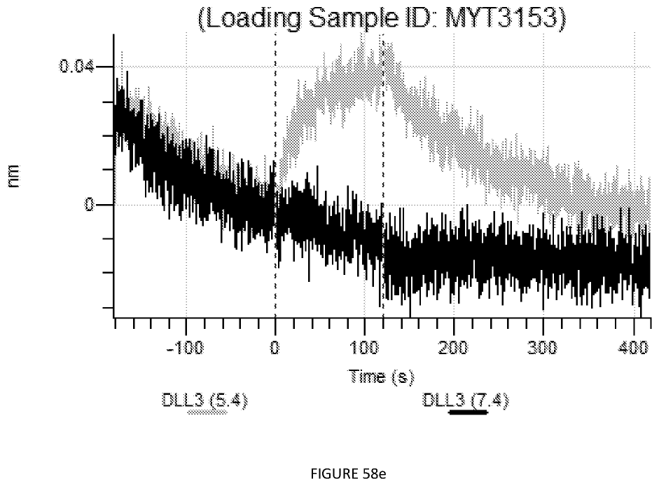

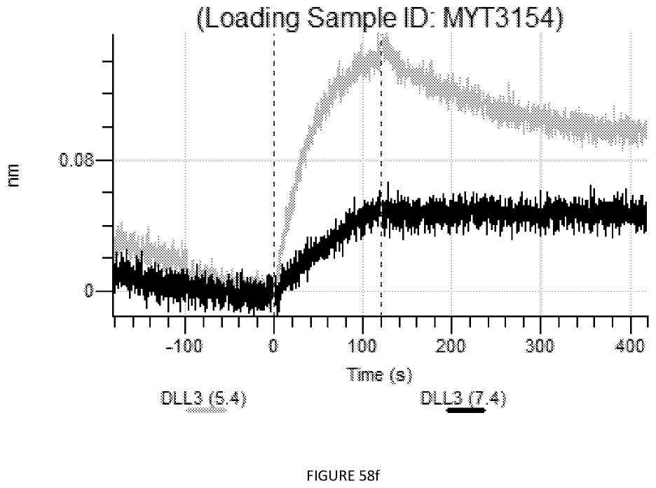

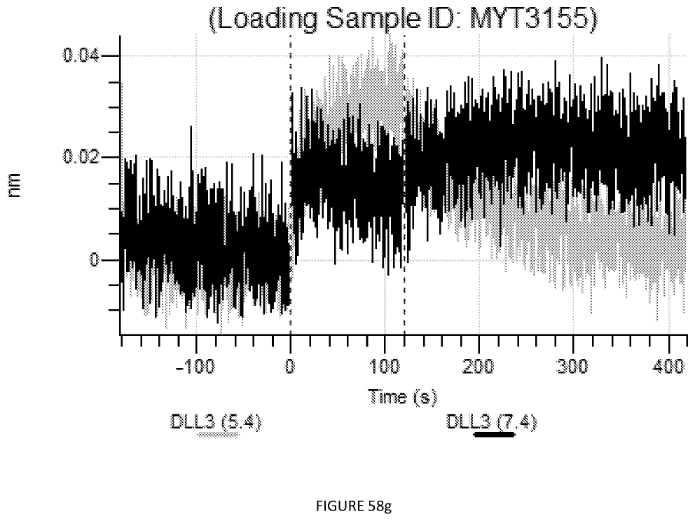

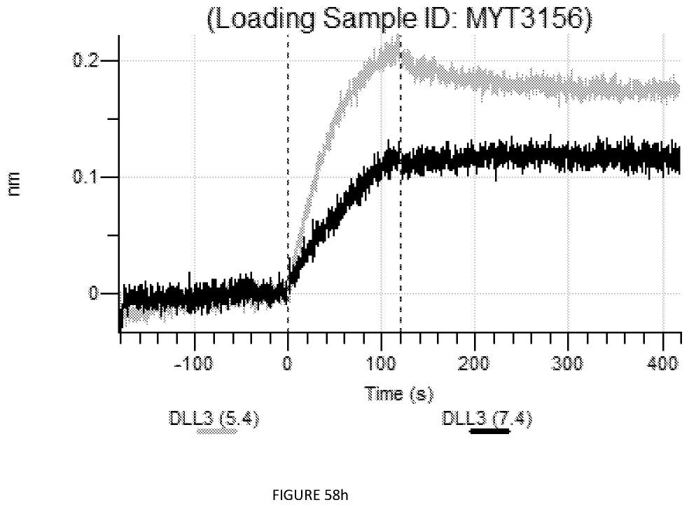

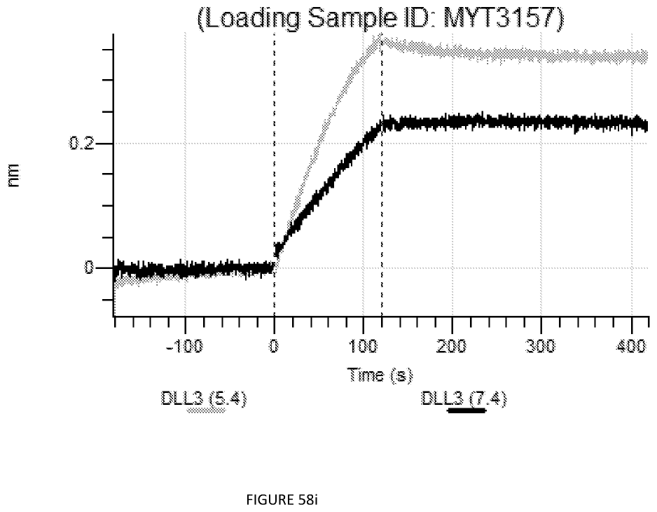

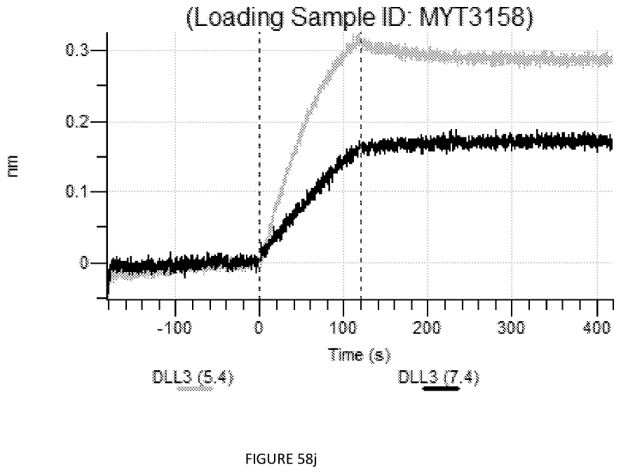

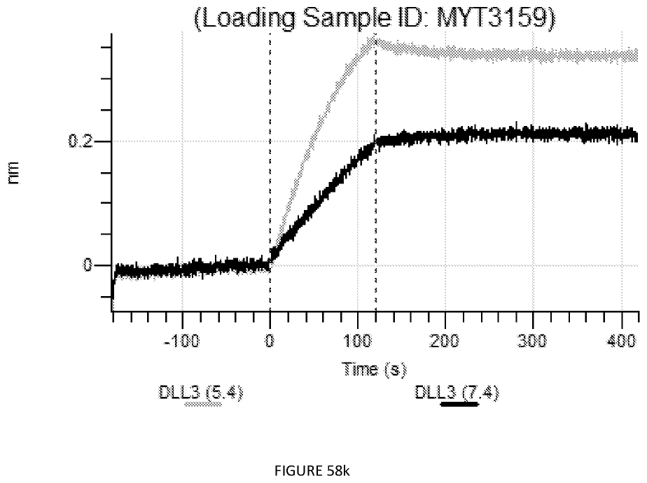

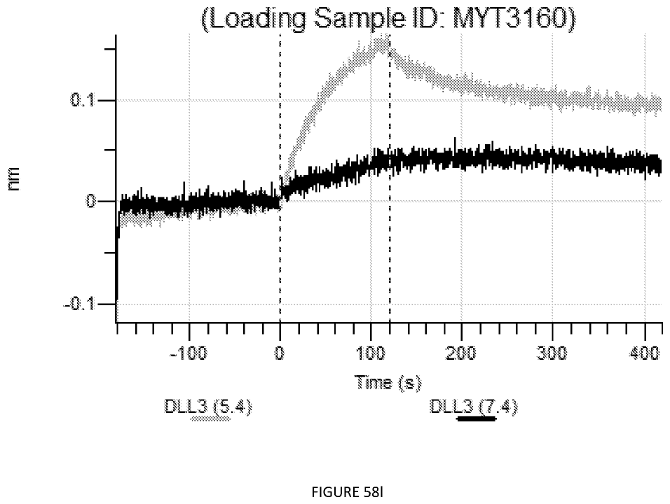

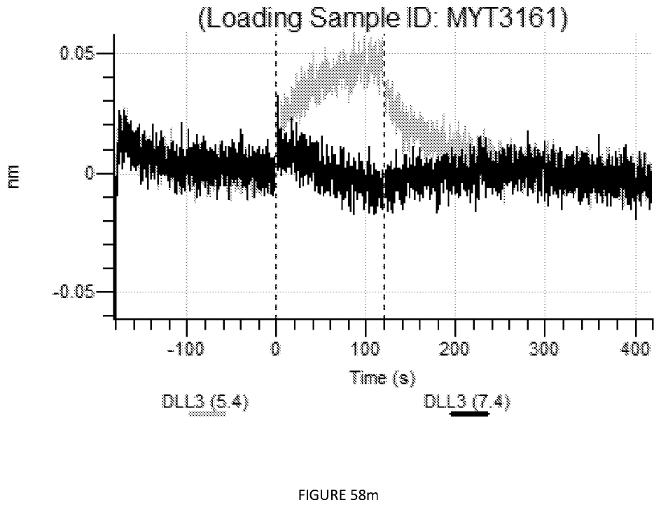

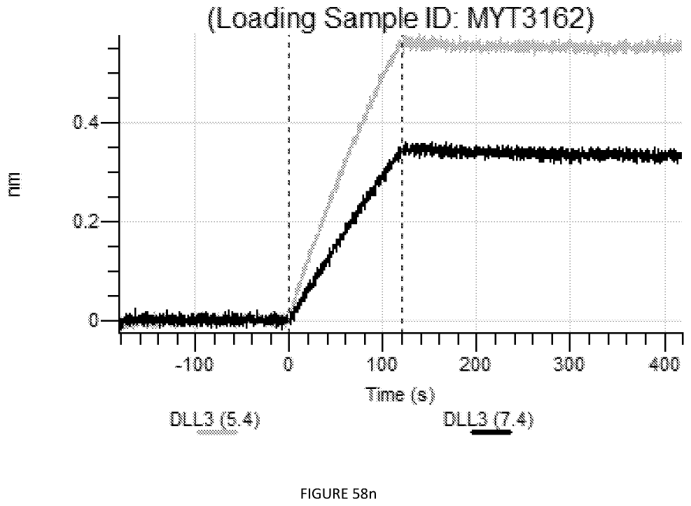

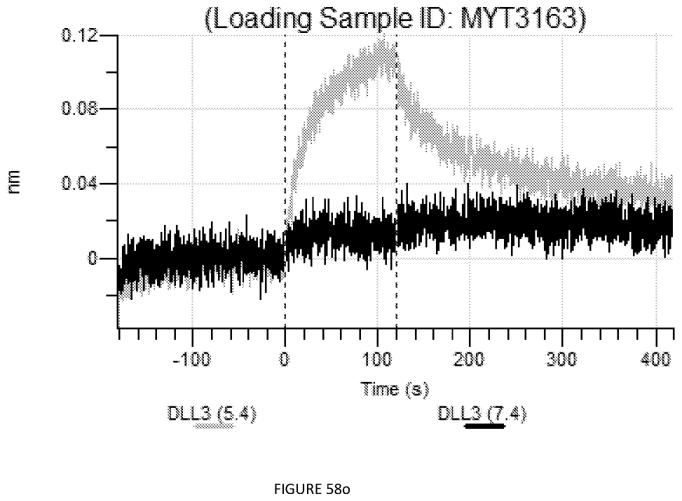

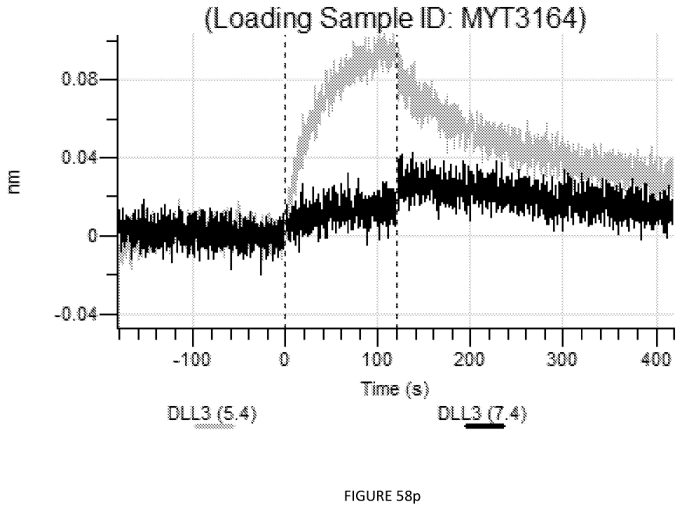

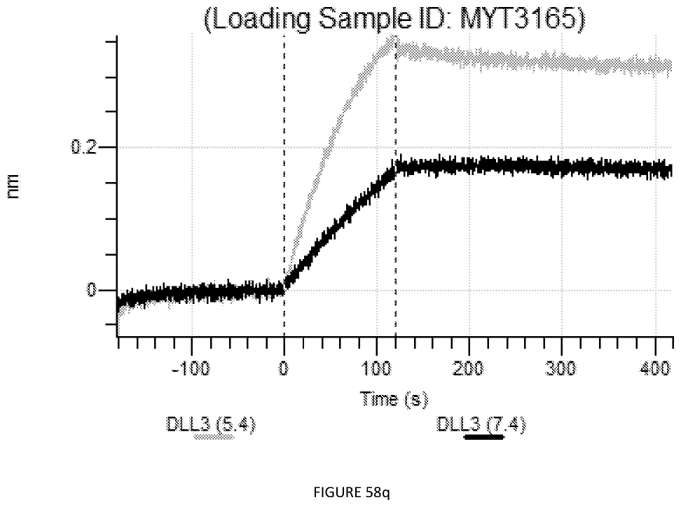

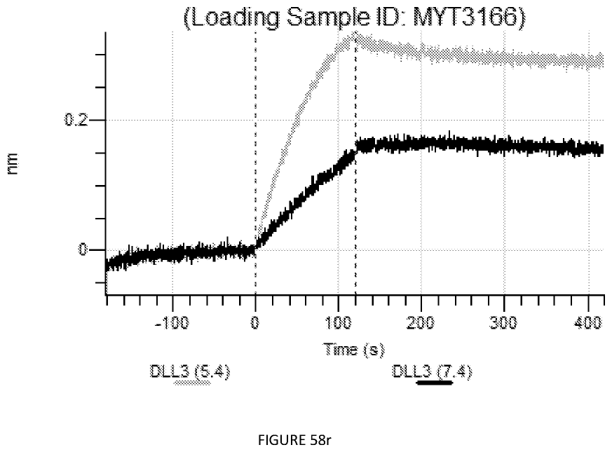

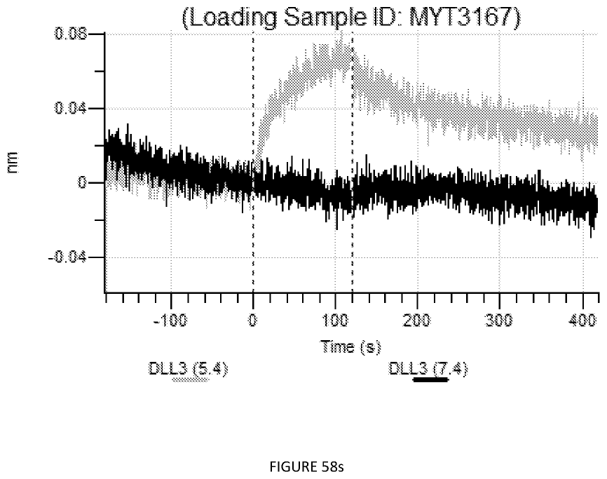

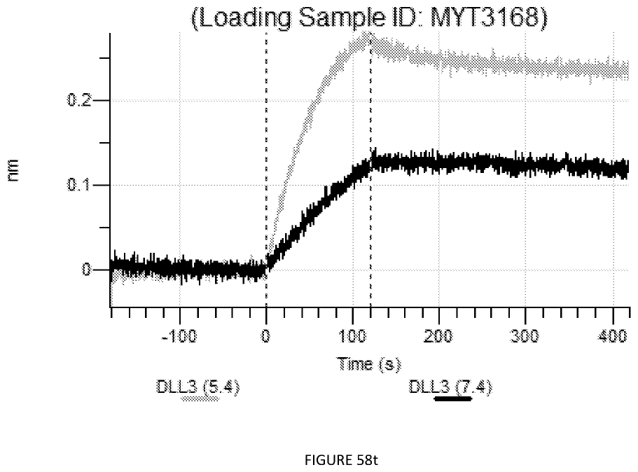

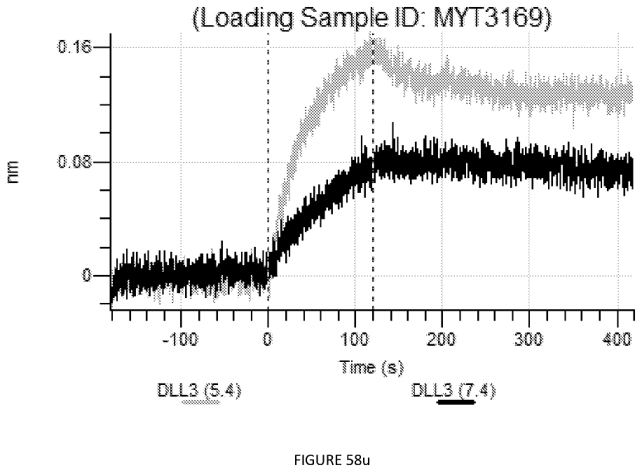

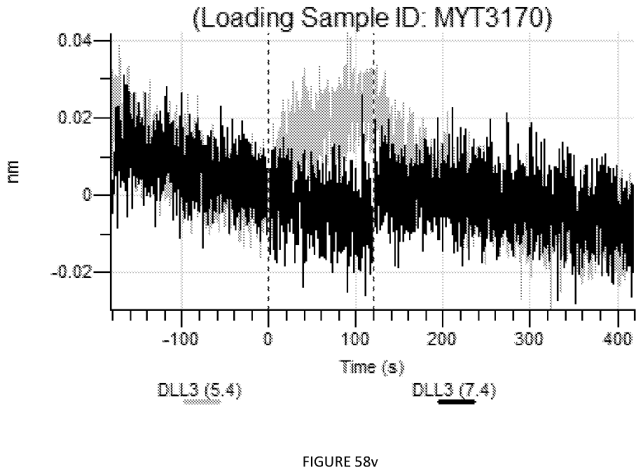

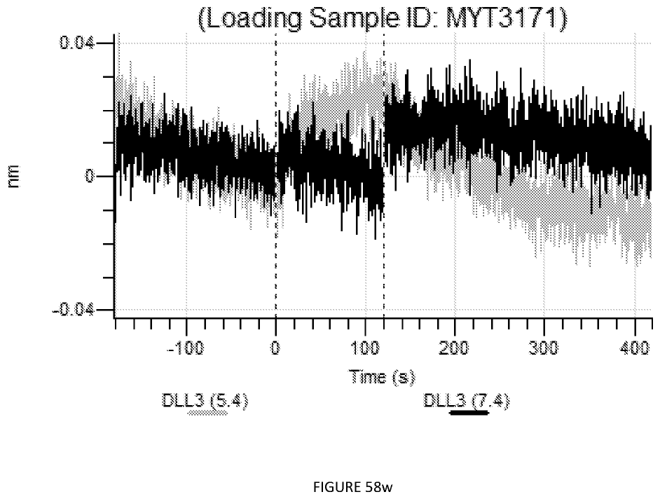

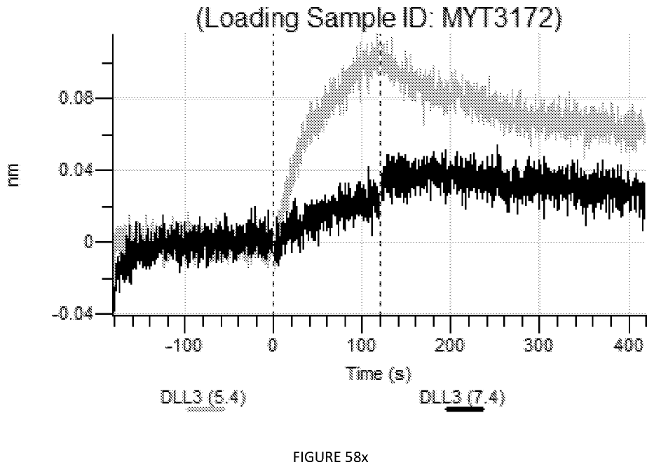

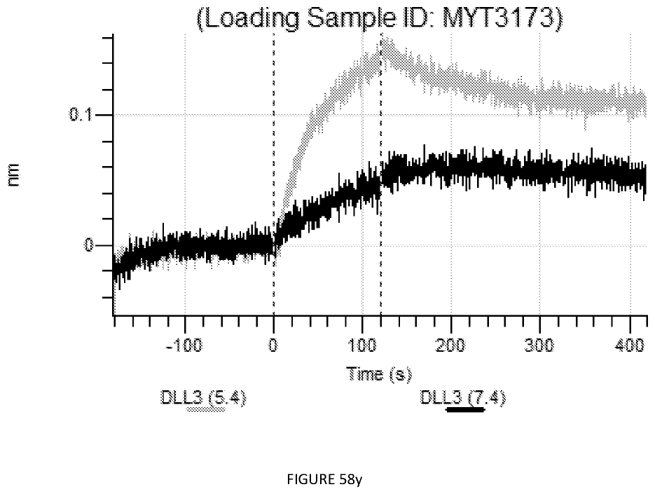

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.67 (U.S. Pat. No. 9,089,617 B2) (Heavy chain SEQ ID NO: 537, Light chain SEQ ID NO: 538) was selected as a DLL3-binding monoclonal antibody for pH engineering via histidine scanning. Briefly, CDRs in the light chain were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid residues within the light chain CDRs were systematically substituted with a histidine, one at a time (MYT3149-MYT3178). In cases where the starting CDR residue was a histidine, it was mutated to an alanine. Antibody variants with only one histidine or alanine mutation in a light chain CDR were generated by co-transfection of Expi293 cells with a) one light chain sequence variant, and b) the corresponding starting ABPC heavy chain using methods known to the art. After allowing for four days of protein expression, cell culture supernatants were collected, quantified by SDS-PAGE analysis (FIG. 57), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Light chain variants that showed either enhanced dissociation at pH 5.4 or reduced dissociation at pH 7.4 or both (as compared to the starting ABPC) as shown in FIG. 58 were selected for further analysis (e.g., MYT3150, MYT3151, MYT3153, MYT3154, MYT3160, MYT3161, MYT3163, MYT3164, MYT3167, MYT3172, MYT3173, and MYT3177). It was also noted that some histidine and alanine mutations were tolerated with little (e.g., less than 1-fold change in dissociation constant KD or dissociation rate) or no change in DLL3 binding kinetics (e.g., MYT3149, MYT3152, MYT3156, MYT3157, MYT3158, MYT3159, MYT3162, MYT3165, MYT3166, MYT3168, MYT3169, MYT3172, MYT3173, MYT3174, MYT3175, MYT3176, and MYT3178). Especially because histidine is a large, positively charged amino acid, these variants with no change were noted as positions in the light chain that may tolerate a wide range of mutations and lead to antibodies with different sequence but similar binding properties, a designation that is not otherwise apparent.

### Example 32. Construction and Screening of pH-Engineered DLL3 ABPCs

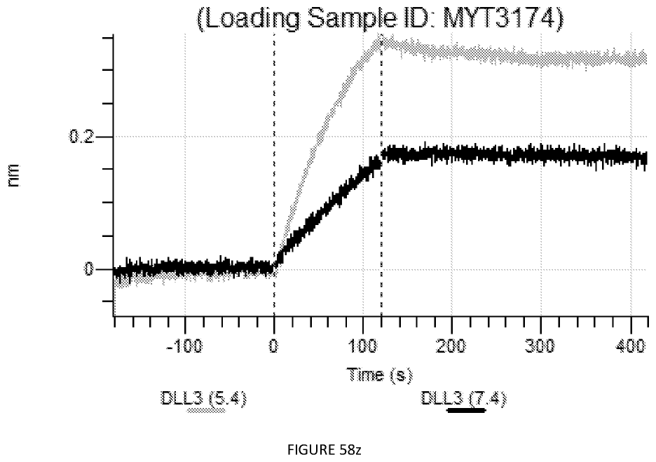

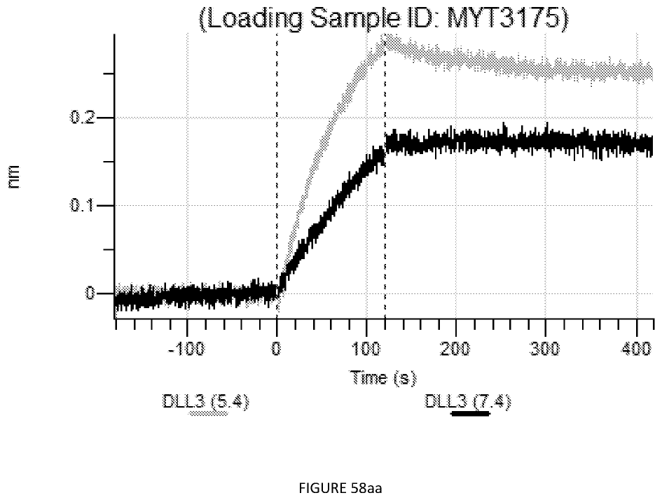

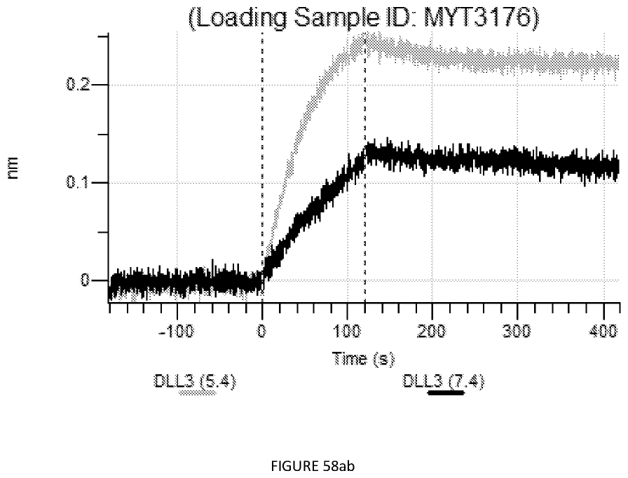

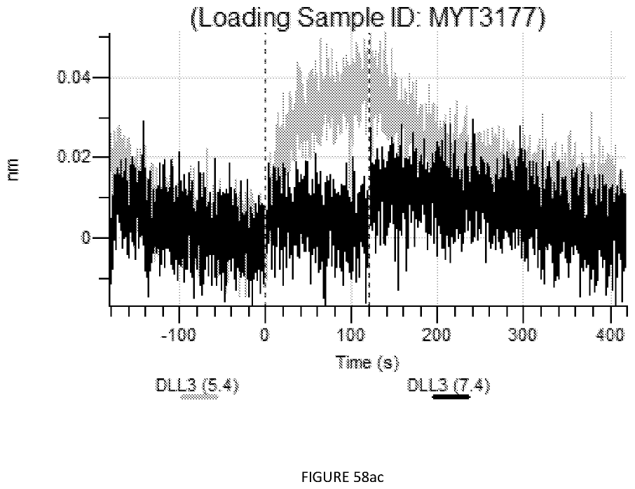

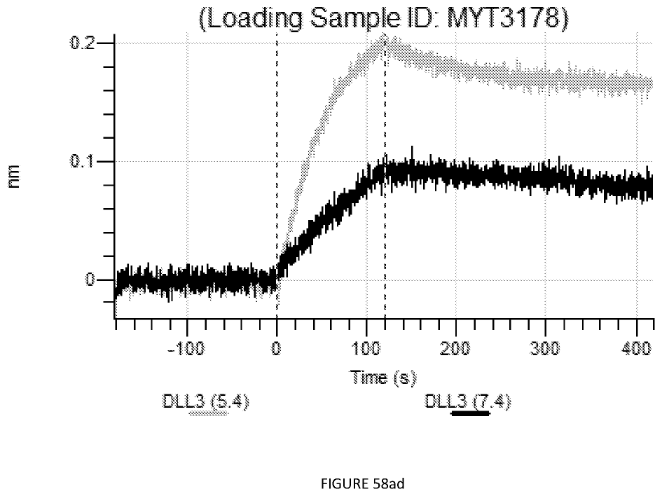

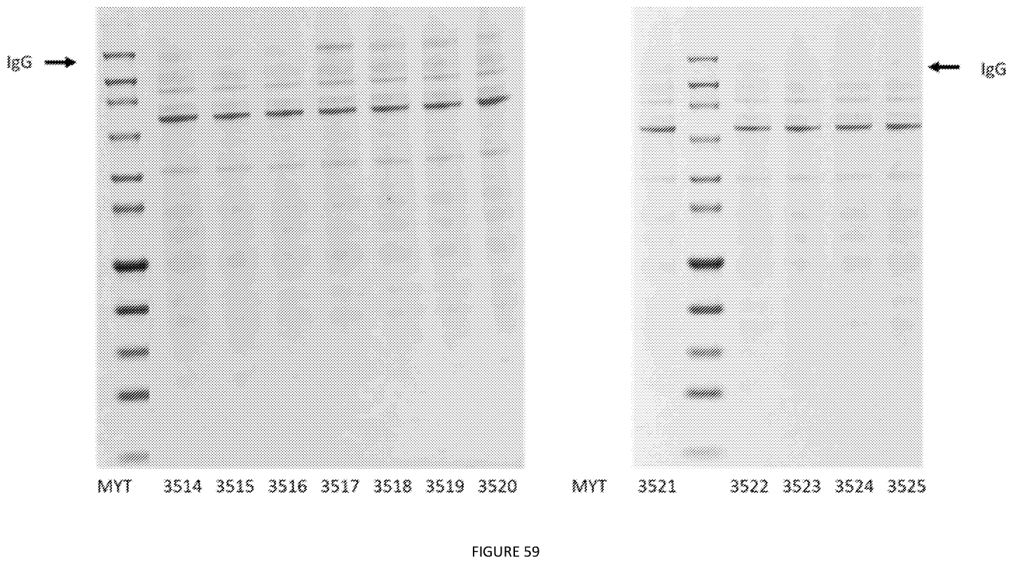

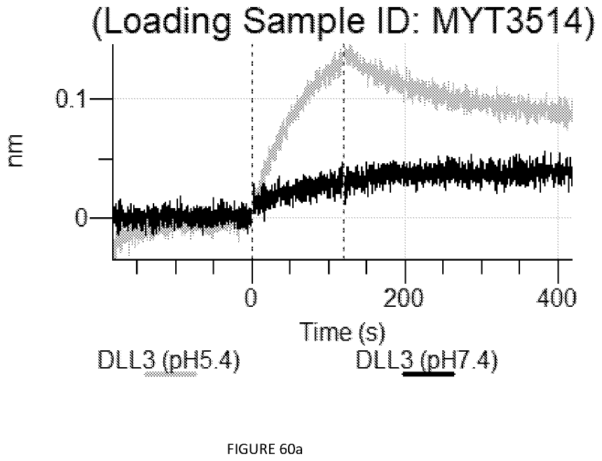

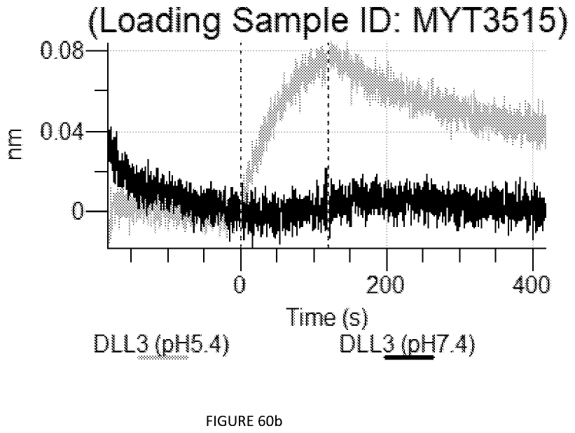

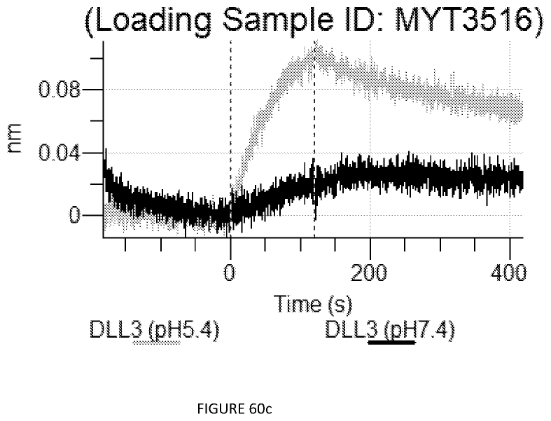

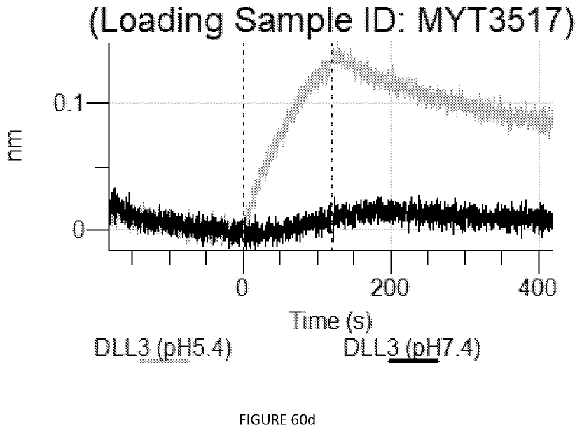

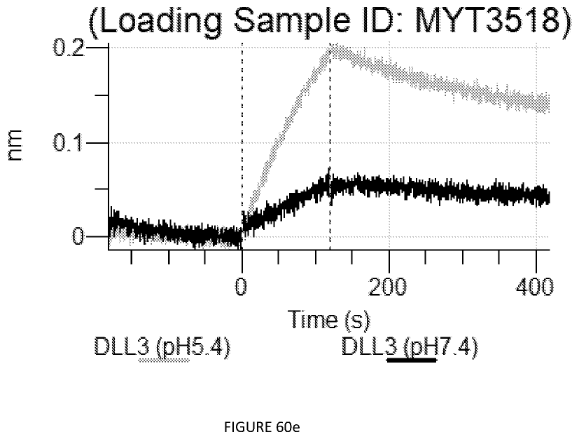

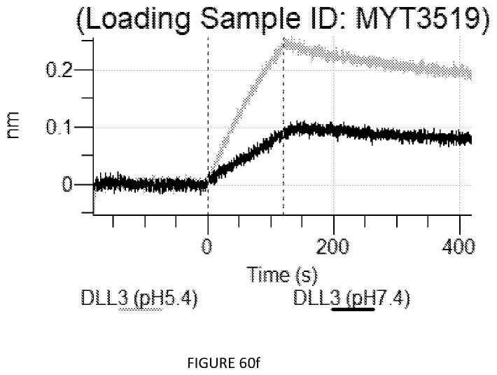

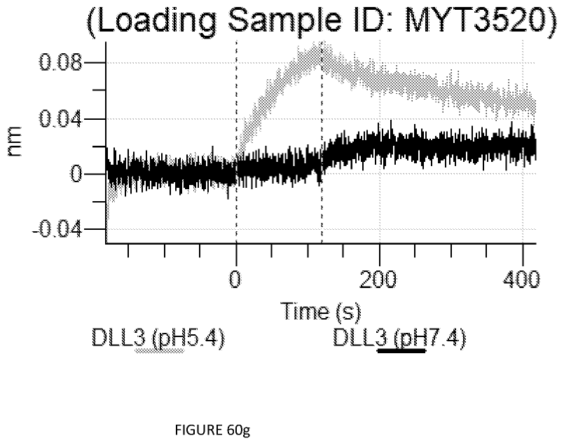

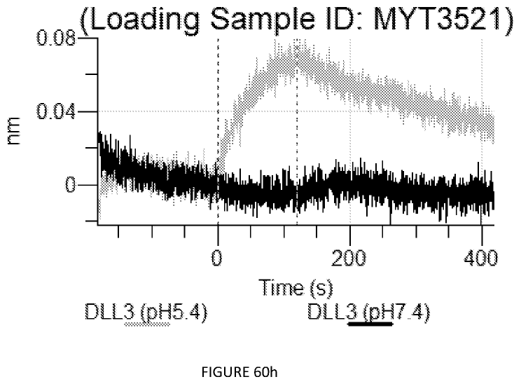

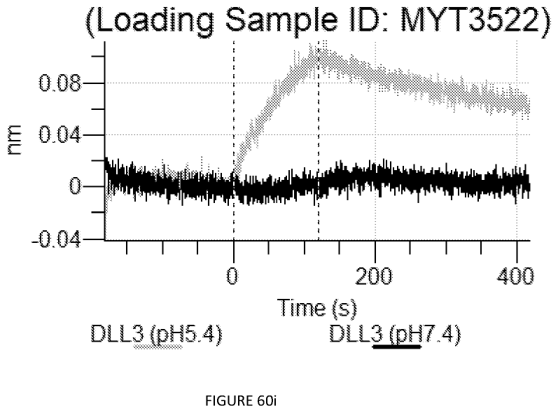

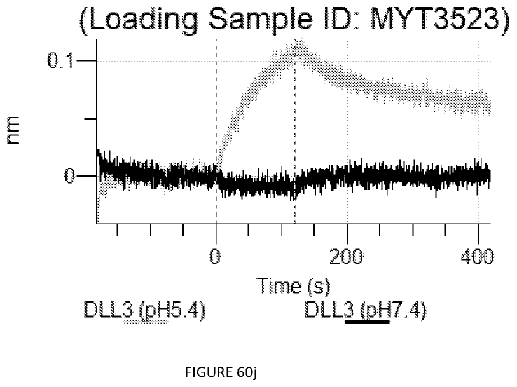

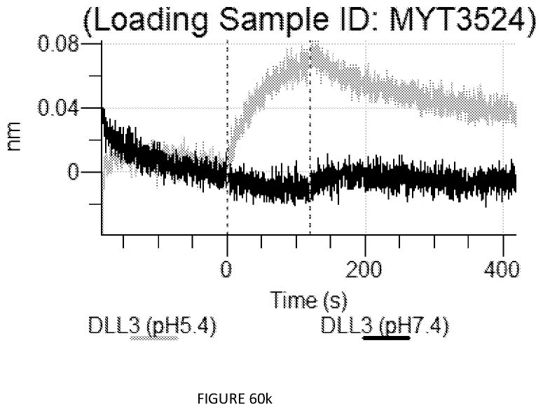

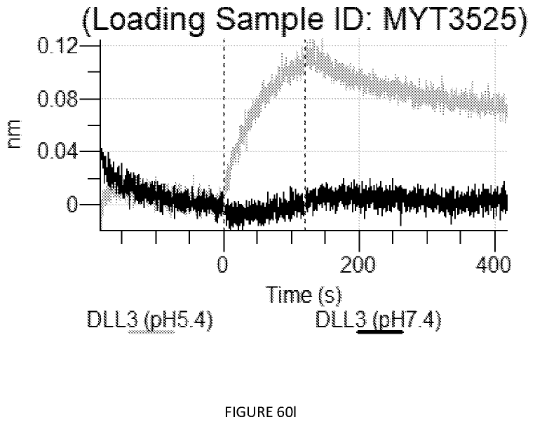

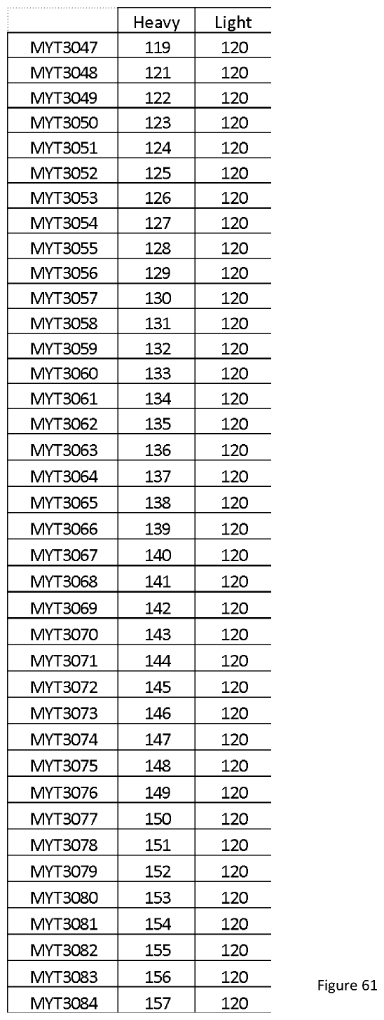

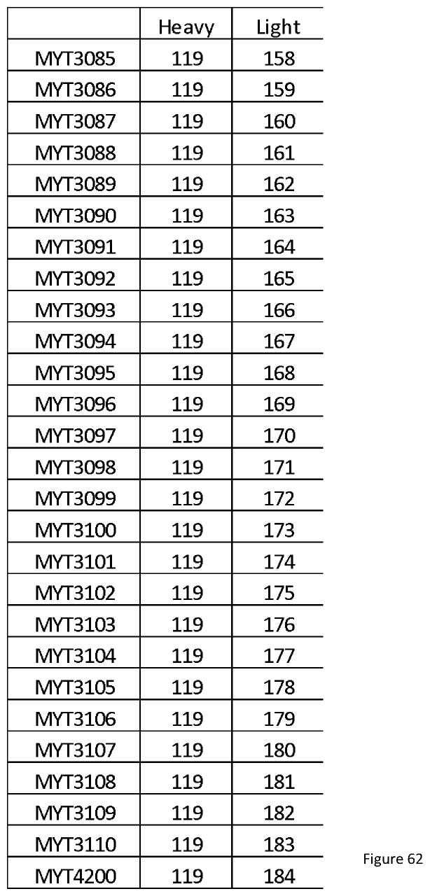

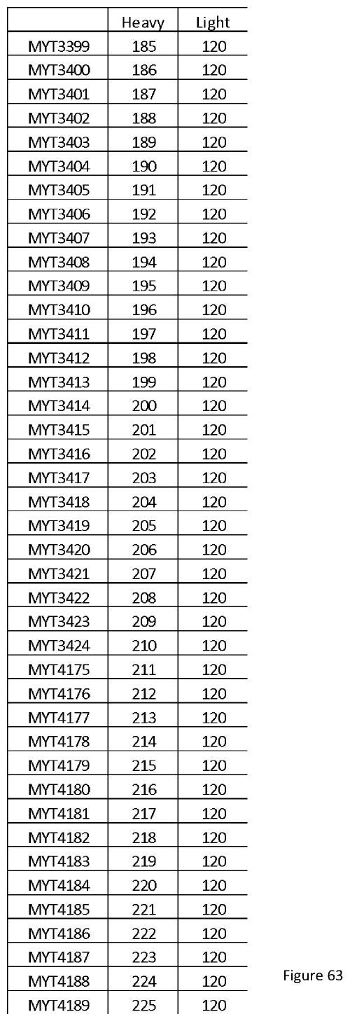

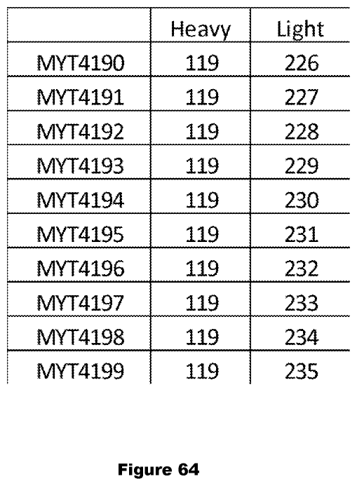

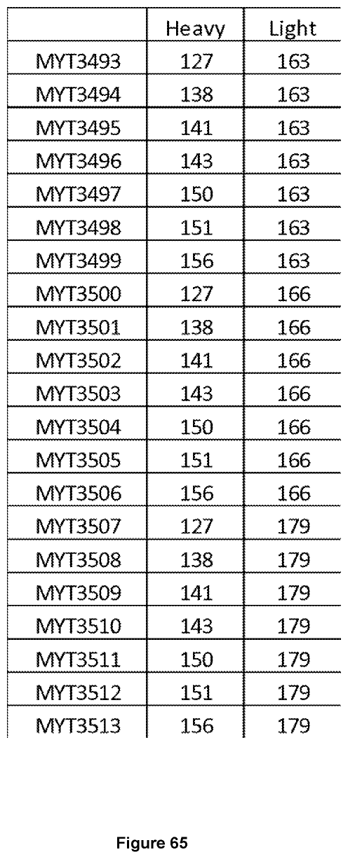

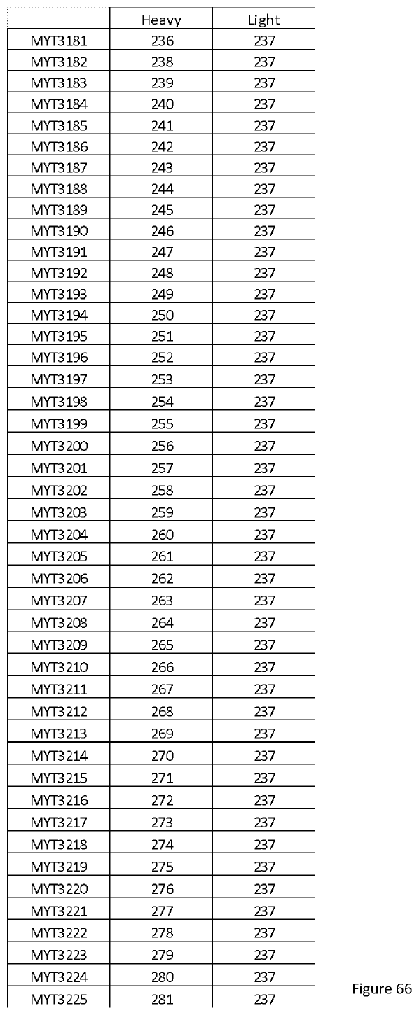

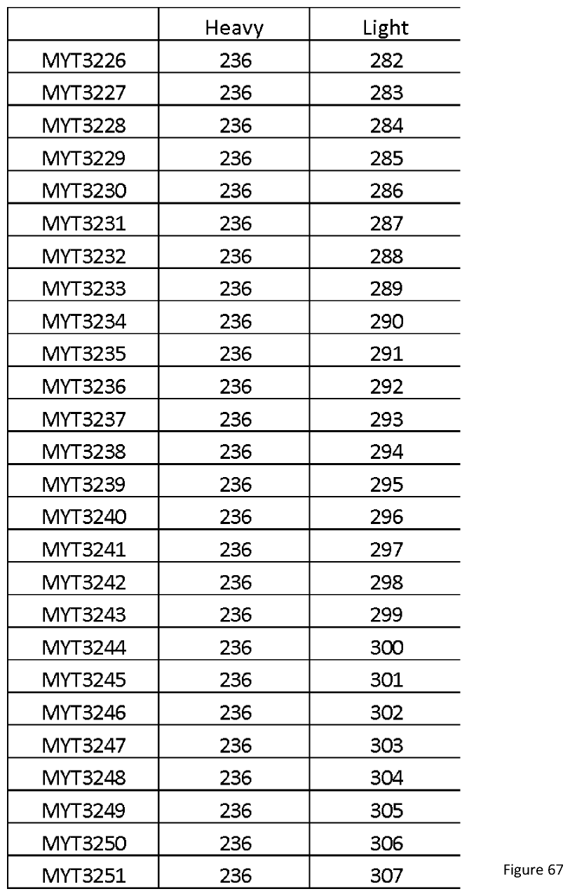

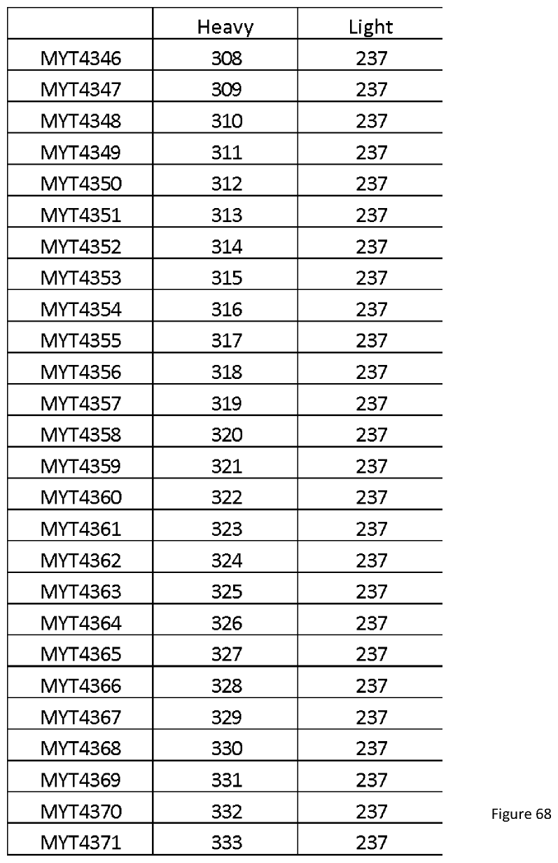

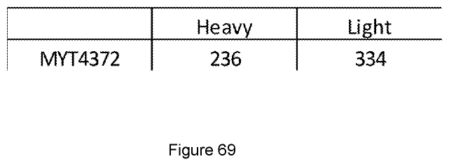

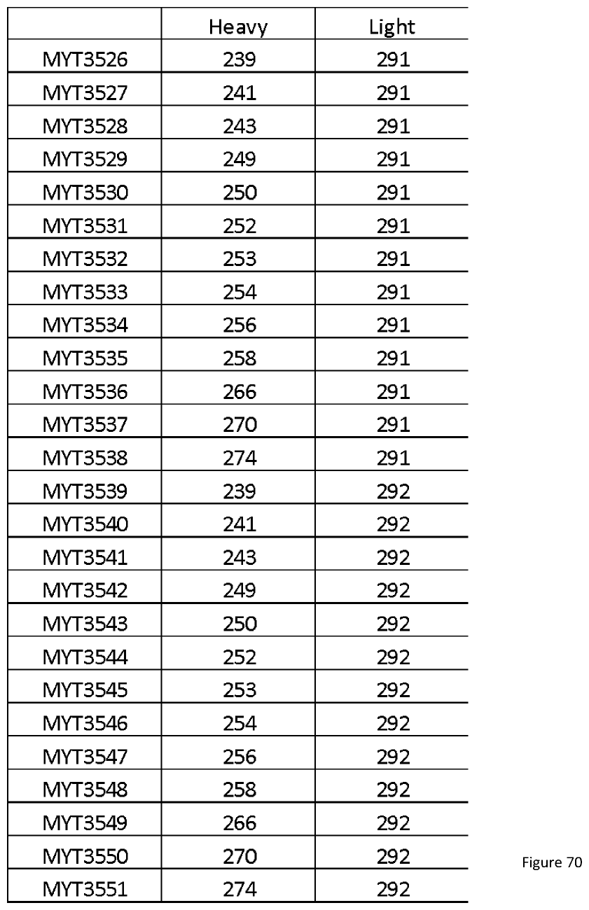

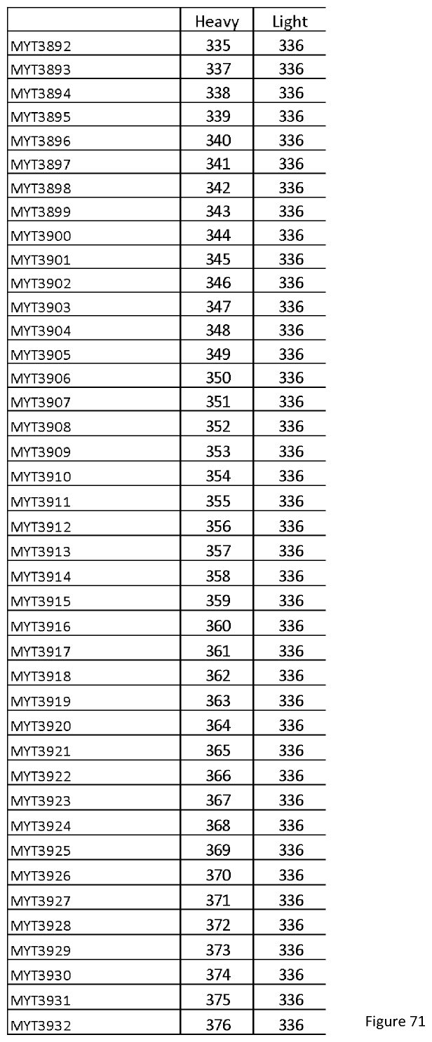

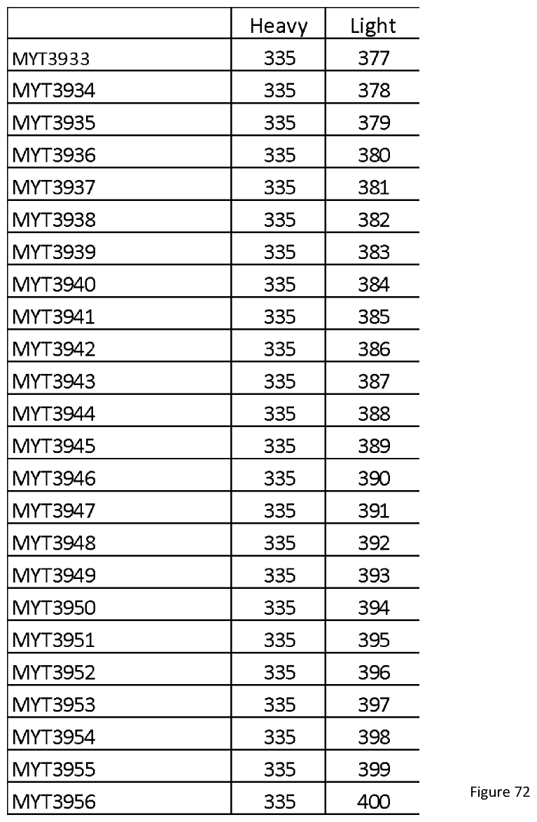

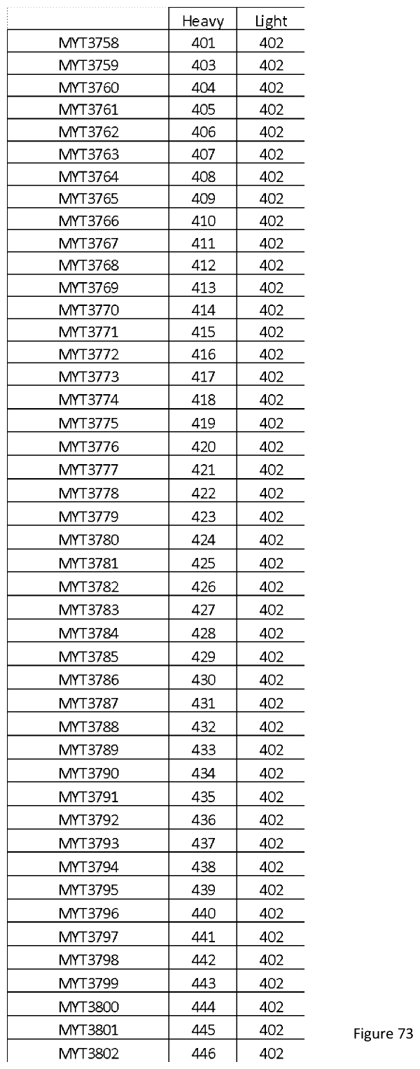

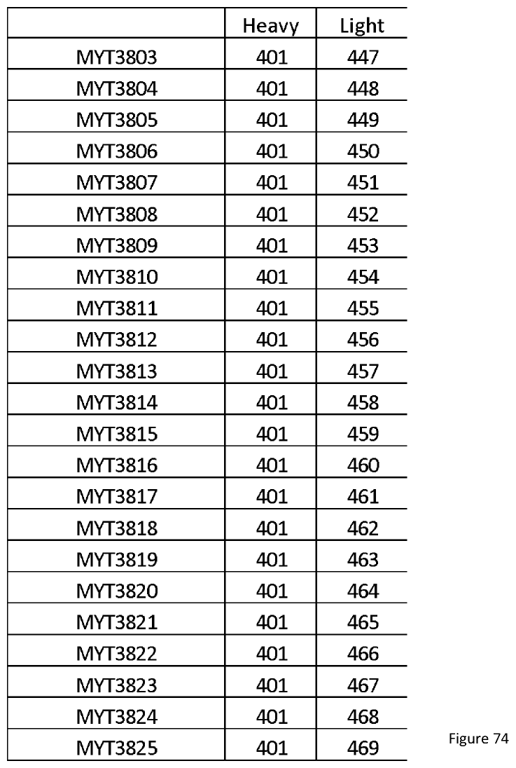

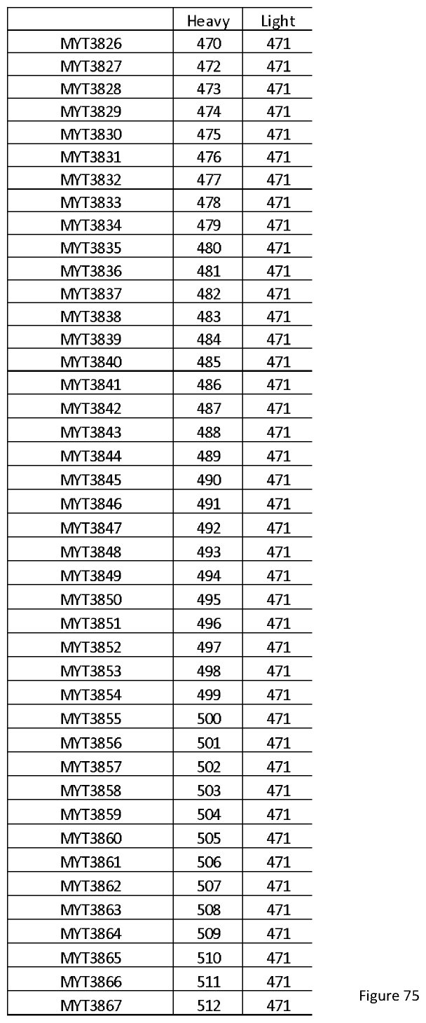

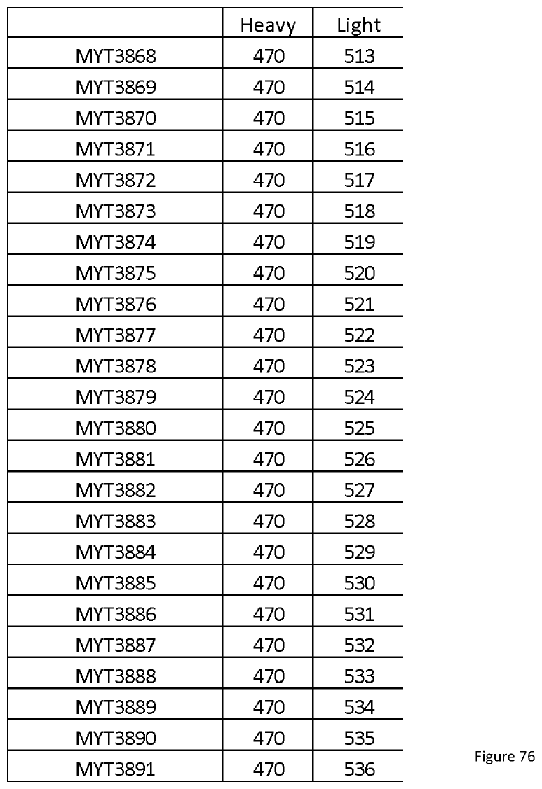

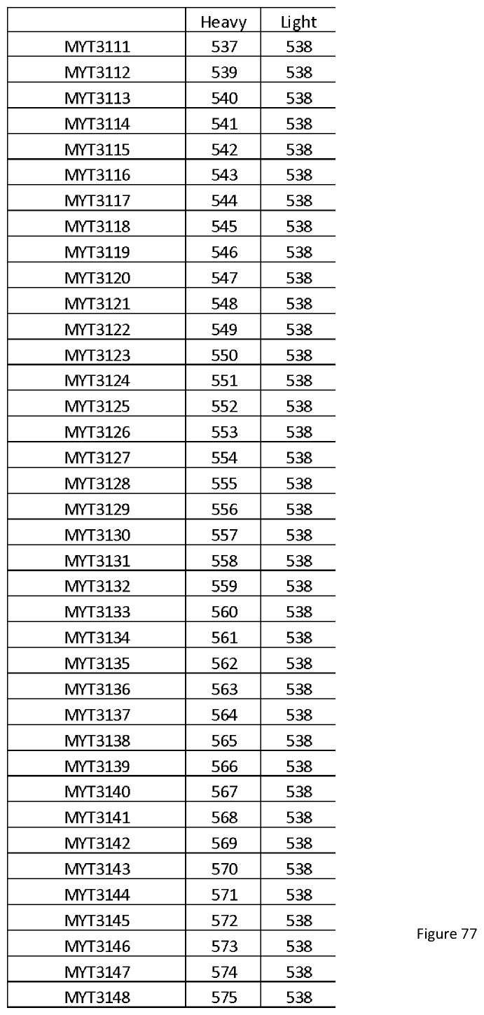

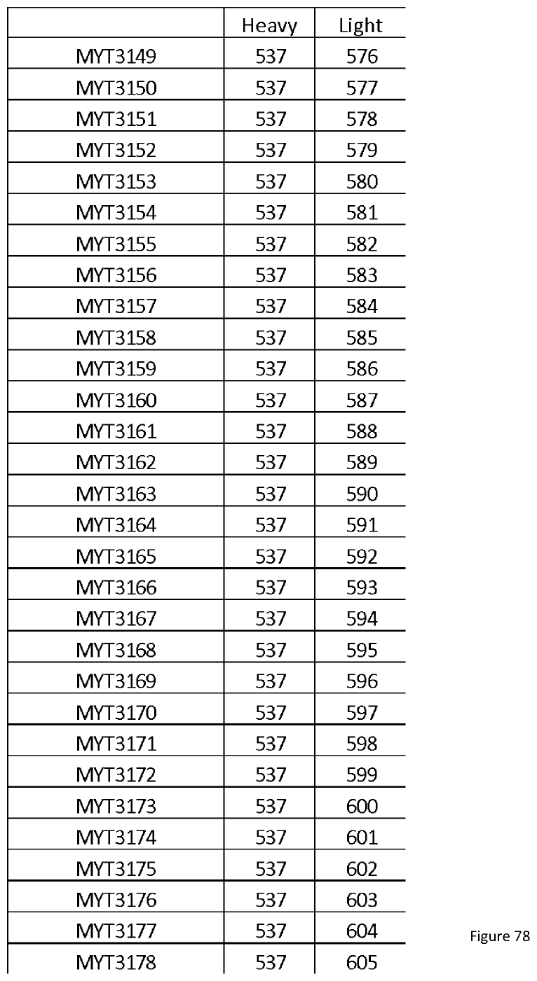

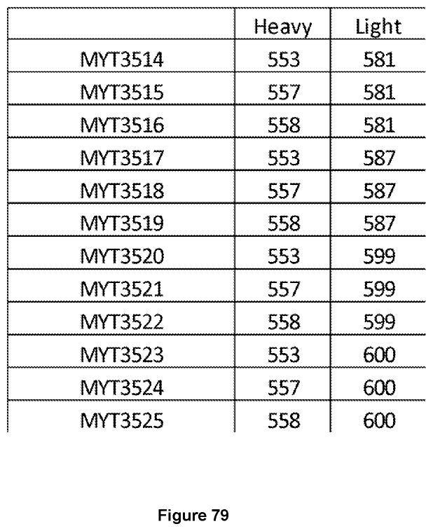

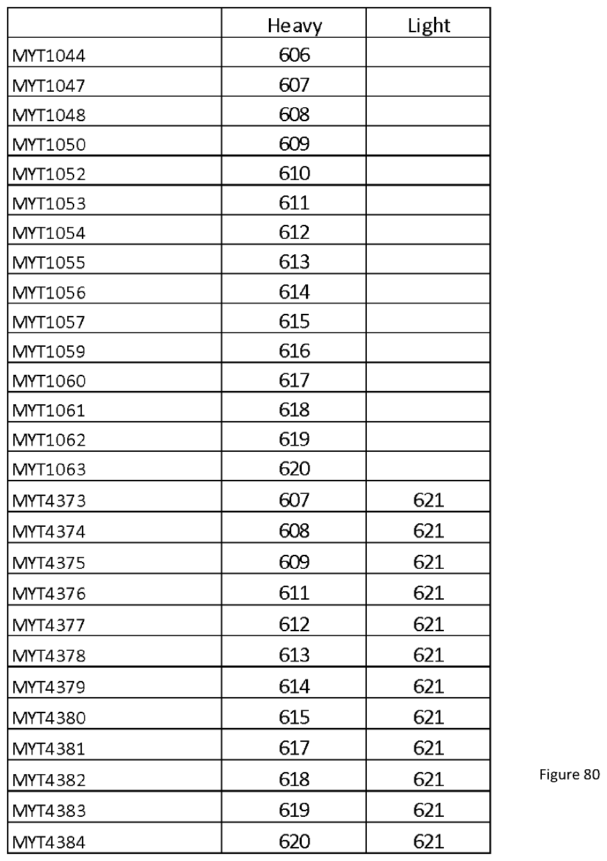

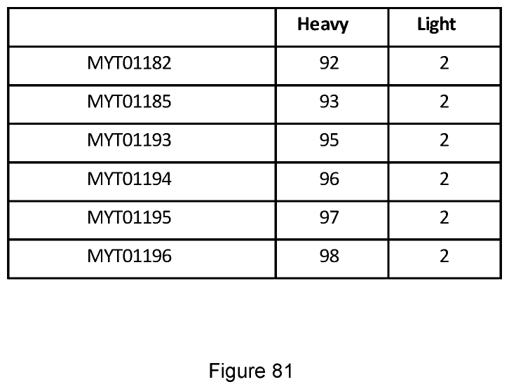

Multiple DLL3-binding monoclonal antibodies have been described in the literature and can be used as a template for engineering pH-dependent binding (Saunders L R (2016) “A DLL3-targeted antibody-drug conjugate eradicates high-grade pulmonary neuroendocrine tumor-initiating cells in vivo” Science Translational Medicine 7(302):302ra136). SC16.67 (U.S. Pat. No. 9,089,617 B2) (Heavy chain SEQ ID NO: 537, Light chain SEQ ID NO: 538) was selected as a DLL3-binding monoclonal antibody for pH engineering via histidine scanning. Briefly, CDRs in the heavy and light chains were identified using the methods described by Kabat et al (Kabat et al. (1992) Sequences of Proteins of Immunological Interest, DIANE publishing) and IMGT (Lefranc M P (1999) “The IMGT unique numbering for Immunoglobulins, T cell receptors and Ig-like domains” The Immunologist 7, 132-136), and for each CDR, residues falling under either or both Kabat and IMGT CDR definitions were called as CDR residues. To generate pH-dependent sequence variants, individual amino acid mutations within the heavy and light chain CDRs that had been previously selected for further analysis in Examples 30 and 31 were systematically combined two or more at a time (MYT3514-3525). In cases where the starting CDR residue was a histidine, it was mutated to an alanine. Antibody variants with two or more histidine or alanine mutations were generated by co-transfection of Expi293 cells with a) one light chain sequence variant, and b) one heavy chain sequence variant using methods known to the art. After allowing for four days of protein expression, cell culture supernatants were collected, quantified by SDS-PAGE analysis (FIG. 59), and the pH dependence of the variant was evaluated using biolayer interferometry (BLI) on an Octet RED 96e instrument. Briefly, cell culture supernatants were diluted based on qualitative expression level of the variant determined by visual examination of SDS-PAGE gels, 5 μL of cell culture supernatant was diluted into 195 μL of 1×PBST, pH 7.4 for high expressors, 25 μL of cell culture supernatant was diluted into 175 μL of 1×PBST, pH 7.4 for medium expressors and 100 μL of cell culture supernatant was diluted into 100 μL of 1×PBST, pH 7.4 for low expressors for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 50 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for the starting ABPC antibody (with no substitutions) and each corresponding antibody variant at pH 5.4 and pH 7.4 to inform on two criteria: a) enhanced dissociation (e.g., higher koff values) at pH 5.4 due to histidine or alanine substitution compared to the starting ABPC (with no substitutions), and b) reduced dissociation at pH 7.4 (e.g., lower koff values) compared to pH 5.4 in the antibody variant itself and with the starting ABPC (with no substitutions). Paired heavy and light chain variants that showed either enhanced dissociation at pH 5.4 or reduced dissociation at pH 7.4 or both (as compared to the starting ABPC), as shown in FIG. 60, were selected for further analysis (MYT3514-3525).

### Example 33. Characterization of Cellular Internalization and Endolysosomal Delivery of pH Engineered Anti-DLL3 ABPCs

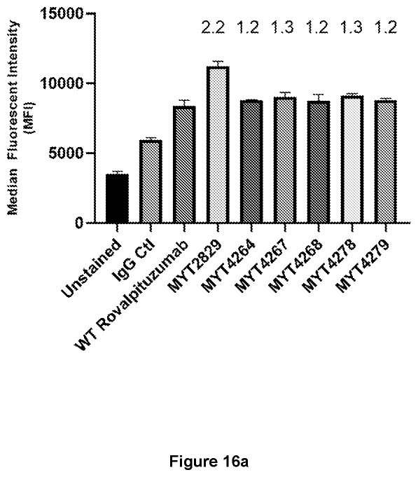

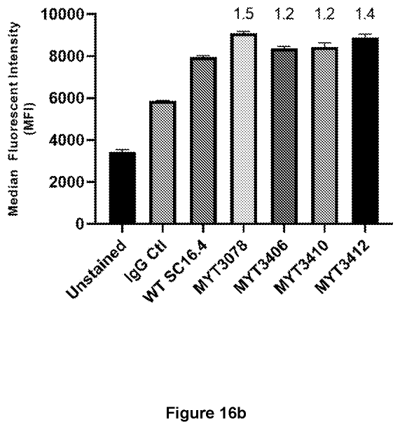

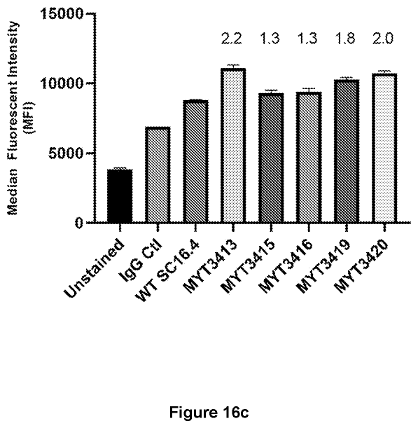

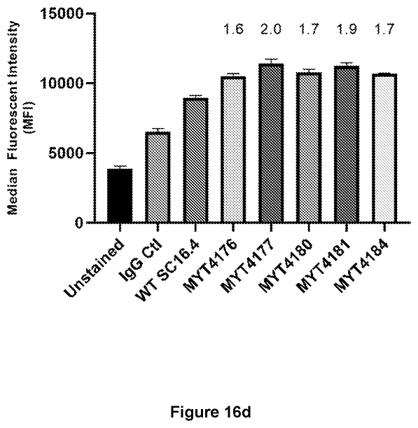

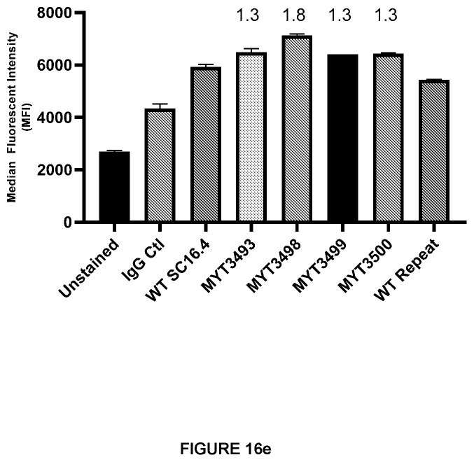

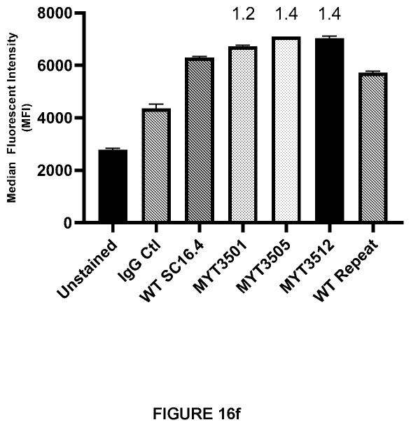

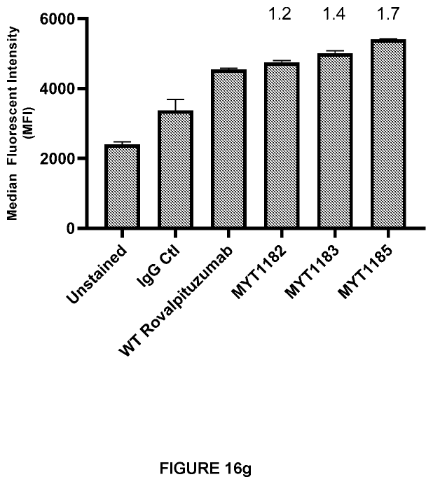

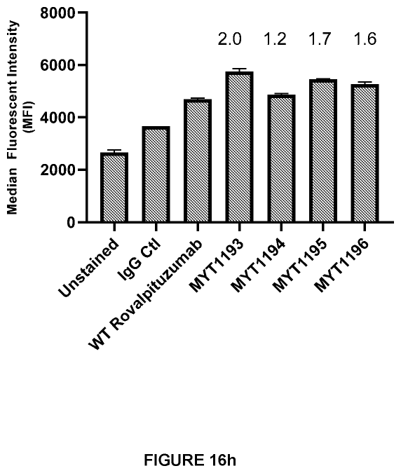

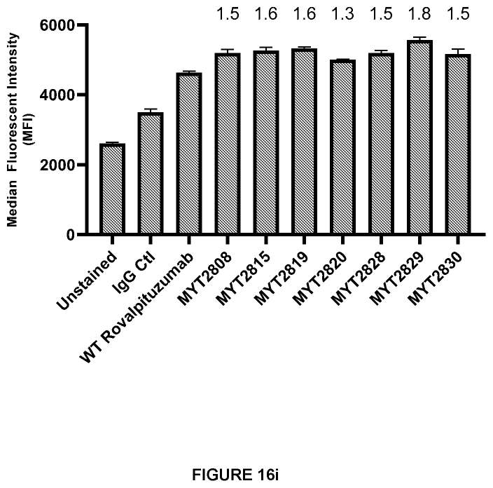

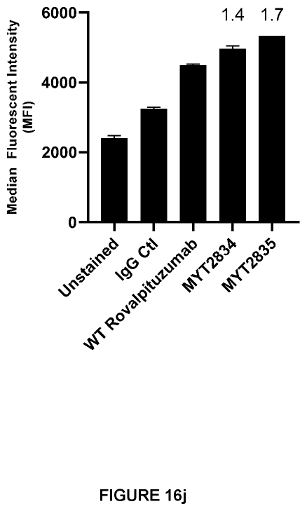

Selected anti-DLL3 pH engineered antibody variants from Examples 9-32 were analyzed for internalization and endolysosomal delivery in NCI-H82 (DLL3+) cells. NCI-H82 cells (ATCC; HTB-175) were collected and resuspended in RPMI-1640 medium (ATCC; 30-2001)+10% GenClone heat inactivated fetal bovine serum (HI FBS) (Genesee Scientific; 25-514H). Cell counts were determined using trypan blue staining and the Countess II FL Automated Cell Counter (Thermofisher; AMQAF1000). Cells were then diluted to 2,000,000 cells/mL and 50 μl/well was seeded into 96-well flat bottom cell culture plates (Genesee Scientific; 25-109). Anti-DLL3 pH engineered antibody variants, starting ABPC antibodies, control IgG1 isotype control (BP0297, Bioxcell), and vehicle control were diluted in native culture media to 100 nM and then mixed 1:1 with 300 nM Zenon pHrodo iFL Human IgG Labeling Reagent (ThermoFisher; Z25611). The mixture was incubated for 20 minutes at room temperature, followed by addition of 50 μL to cells. The mixture of cells, anti-DLL3 antibody variants, and Zenon pHrodo iFL Human IgG Labeling Reagent was incubated at 37° C., 5% C02 for 1-24 hours. Following incubation, 100 μL of ice cold Flow Cytometry (FC) buffer (phosphate buffered saline (PBS), pH 7.4+2 mM ethylenediaminetetraacetic acid (EDTA)+2% (v/v) HI FBS is added to each well. Cells were then spun down at 4° C. for 2 min at 2000 rpm, washed with 200 μL ice cold FC buffer and resuspended in 100 μL ice cold FC buffer. Mean green fluorescence intensity was detected using a BD Accuri C6 flow cytometer. Data was analyzed using Flowjo analysis software. pHrodo green is a pH sensitive dye that fluoresces in the low pH environment of the endosomes and lysosomes and therefore can be used to quantify antibody internalization and endolysosomal delivery. Internalization and endolysosomal delivery of anti-DLL3 starting ABPC antibodies rovalpituzumab, SC16.4, hSC16.13, hSC16.15, hSC16.25, hSC16.34, SC16.67 and their variants in NCI-H82 (DLL3+) cells is shown in FIG. 16 as measured by pHrodo green mean fluorescence intensity. Several pH engineered anti-DLL3 antibody variants showed increased mean fluorescence intensity relative to their corresponding starting ABPC antibodies demonstrating that increased dissociation at lower pH leads to enhanced internalization and endolysosomal delivery inside cells as shown by increased fluorescence or increased fluorescence as compared to IgG1 isotype control. Increased endolysosomal delivery is quantitated for each pH engineered anti-DLL3 antibody variant on the top of each bar as a ratio of: the variant's mean fluorescence intensity minus the mean fluorescence intensity of the IgG control, then all divided by the variant's corresponding starting ABPC's mean fluorescence intensity minus the mean fluorescence intensity of the IgG control. For example MYT1182, MYT1183, MYT1193, MYT2808, MYT2829 etc., antibody variants of rovalpituzumab, show increased internalization and endolysosomal delivery relative to rovalpituzumab (MYT0638); similarly for example MYT3078, MYT3410, MYT3413, MYT3419, MYT4176, MYT4184 etc., antibody variants of SC16.4, show increased internalization and endolysosomal delivery relative to SC16.4 (MYT3047) etc. Such pH engineered anti-DLL3 antibody variants with increased mean fluorescence intensity relative to their starting ABPC antibodies were selected for further analysis (MYT1182, MYT1183, MYT1185, MYT1193, MYT1194, MYT1195, MYT1196, MYT2808, MYT2815, MYT2819, MYT2820, MYT2828, MYT2829, MYT2830, MYT2834, MYT2835, MYT4264, MYT4267, MYT4268, MYT4278, MYT4279, MYT3078, MYT3406, MYT3410, MYT3412, MYT3413, MYT3415, MYT3416, MYT3419, MYT3420, MYT3493, MYT3498, MYT3499, MYT3500, MYT3501, MYT3505, MYT3512, MYT4176, MYT4177, MYT4180, MYT4181, MYT4184).

### Example 34. De Novo Selection of Anti-DLL3 pH Engineered Variants

Multiple ABPC display systems, including phage display systems and those with ABPCs based on VH domains, have been described in the literature and can be used as a template for selection of pH engineered ABPCs using panning (Scroter C (2015) “A generic approach to engineer antibody pH switches using combinatorial histidine scanning libraries and yeast display” mAbs, 7:1, 138-151; Bonvin P (2015) “De novo isolation of antibodies with pH-dependent binding properties” mAbs, 7:2, 294-302; Traxlmayr P (2014) “Construction of pH-sensitive Her2-binding IgG1-Fc by directed evolution” Biotechnology Journal 9(8): 1013-1022; Gera N (2012) “Design of pH sensitive binding proteins from the hyperthermophilic Sso7 d scaffold” PLOS ONE 7 (11) e48928). pH engineered anti-DLL3 variants were selected by screening a proprietary humanized VH domain phage display library. Humanized VH library was screened against soluble recombinant human DLL3 extracellular domain (R&D Systems, Cat #9749-DL) in three rounds of selections with successively lower concentrations to isolate pH dependent anti-DLL3 VH domains. Briefly, the library was depleted for non-specific binders by incubating with Dynal M-280 streptavidin beads, bound antibodies were separated via a magnet and supernatant was collected for positive selection with human DLL3. Depleted library was further incubated with biotinylated recombinant human DLL3 at pH 7.4 for 1-3 hours at room temperature. Dynal streptavidin beads were added to the mixture to capture human DLL3 bound antibodies and washed 3× with a pH 7.4 buffer. Bound antibodies were eluted from beads using 0.1 M glycine pH 2.7 which was then neutralized with 1M Tris pH 7.4. This procedure was repeated for rounds 2 and 3 with successively lower concentration of biotinylated human DLL3, increased washing frequency and elution with sodium acetate buffer at pH 5.0. pH dependent antibodies enriched at the end of round 3 were analyzed by phage ELISA, sequenced and positive hits with unique CDR3 amino acid sequences were selected for further analysis (SEQ ID NOs: 606-620)

### Example 35. Anti-DLL3 VH-Fc and VH-Fc Converted to IgG Octet

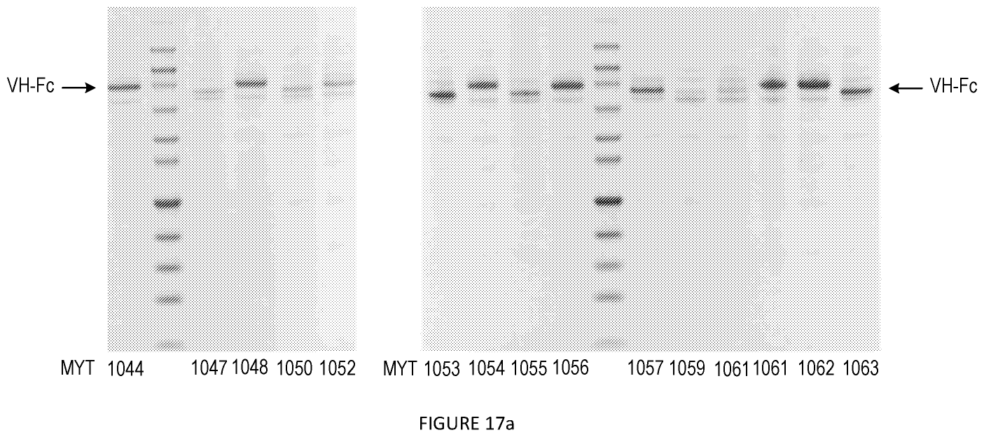

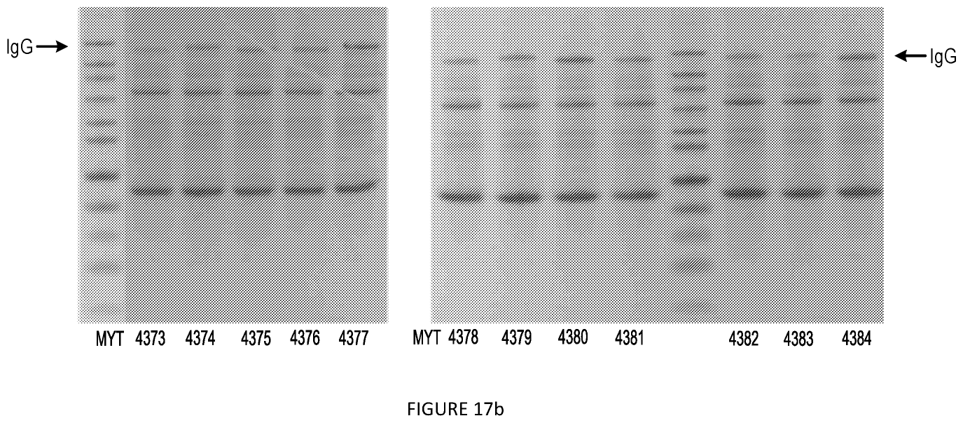

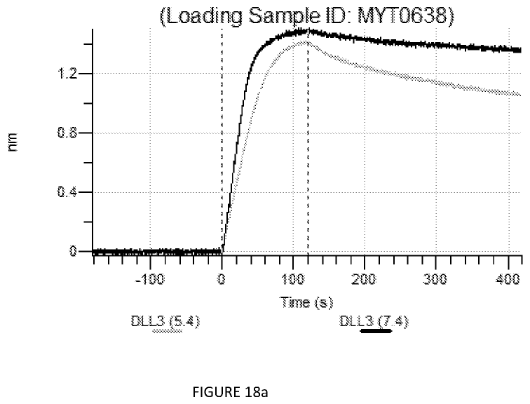

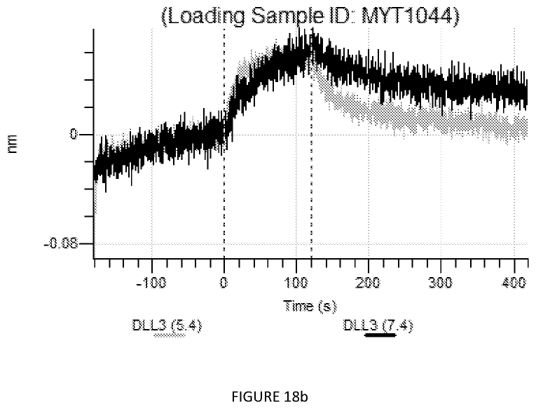

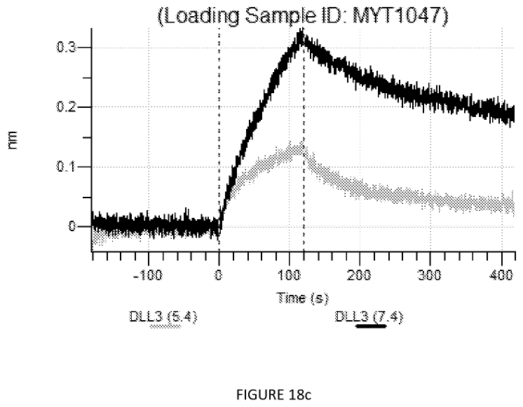

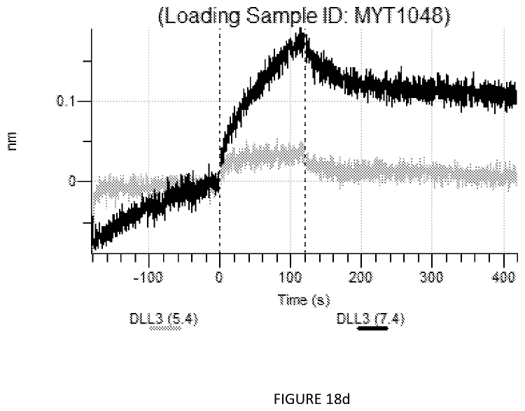

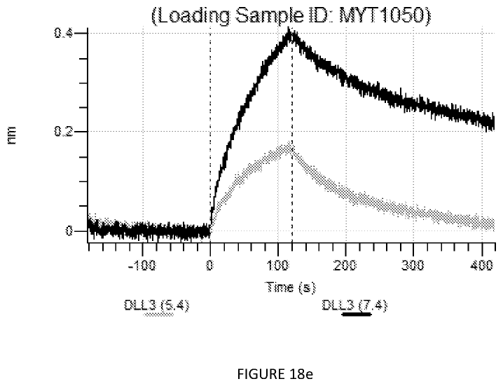

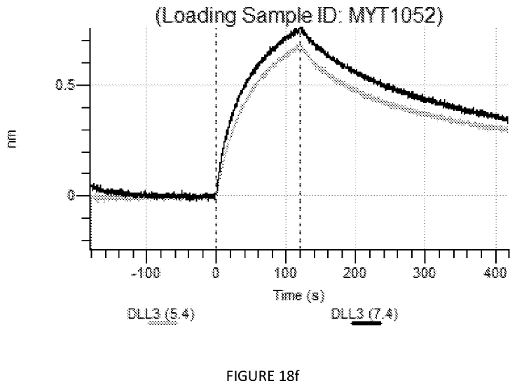

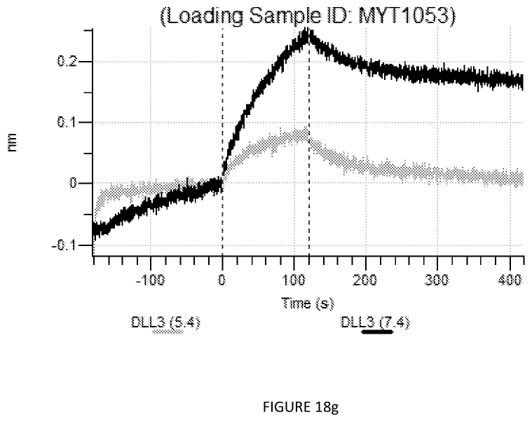

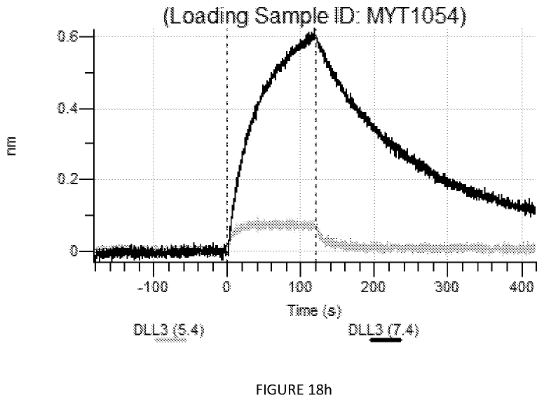

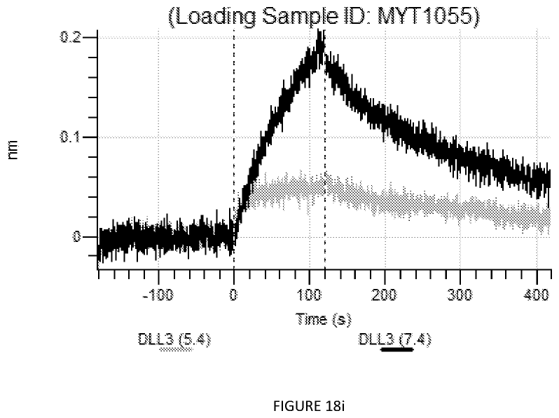

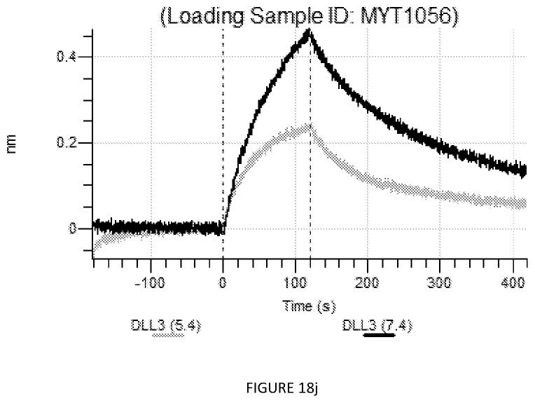

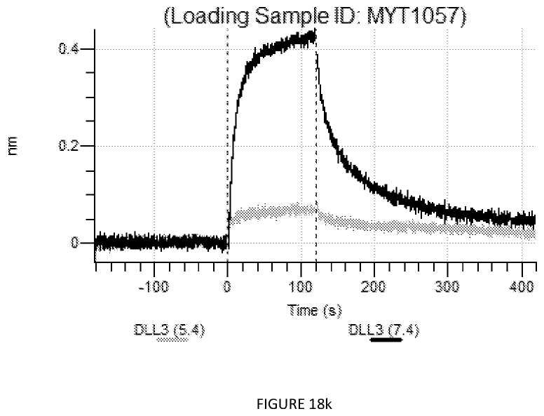

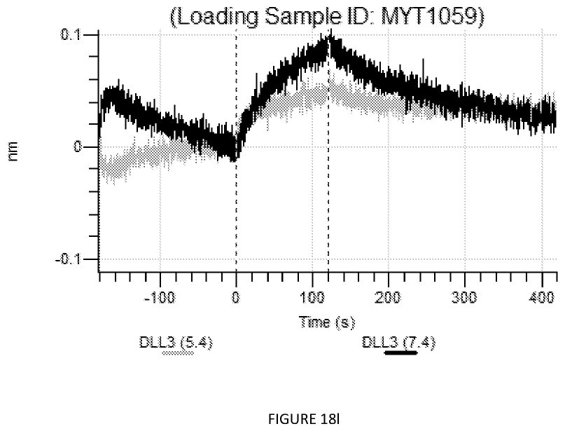

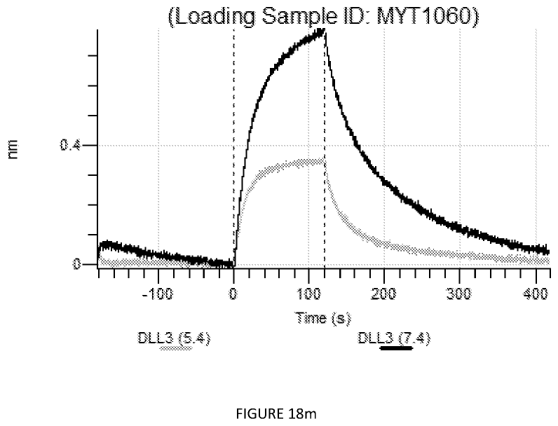

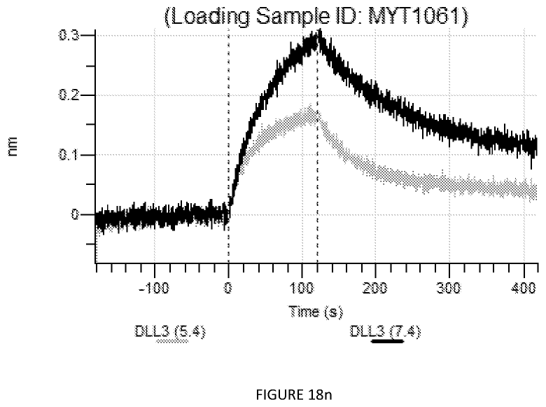

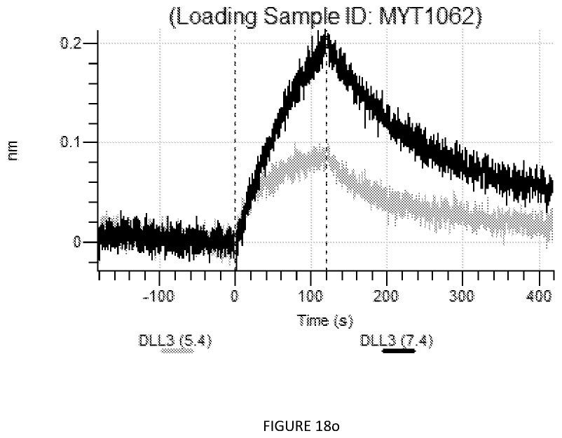

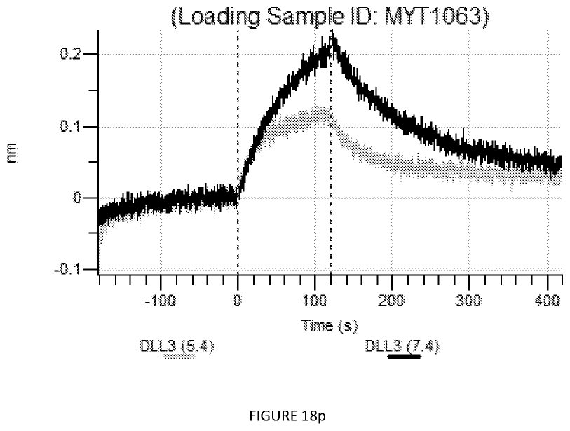

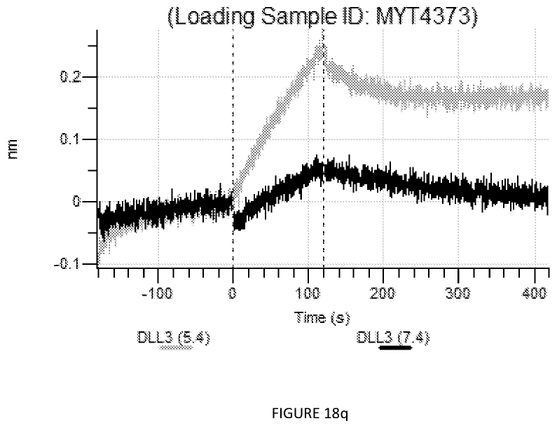

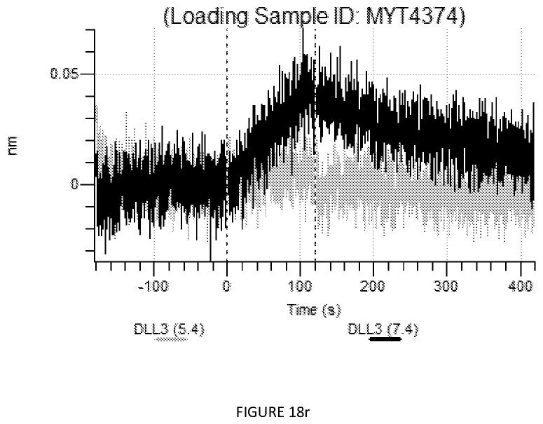

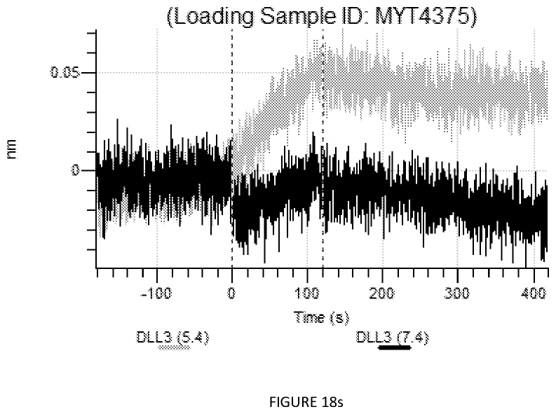

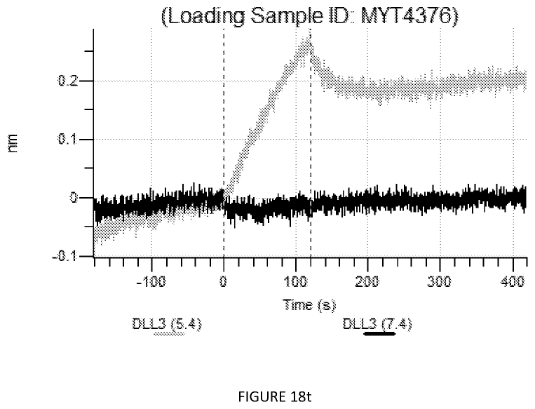

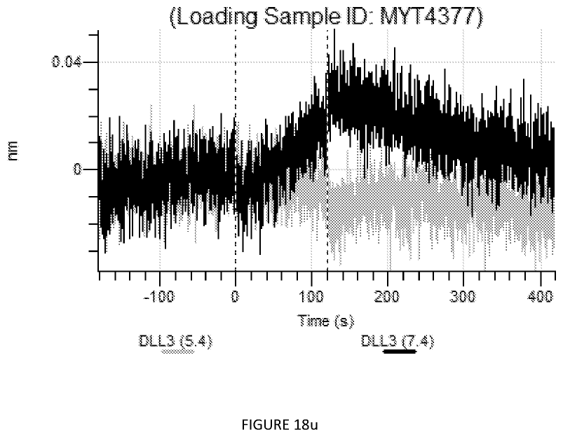

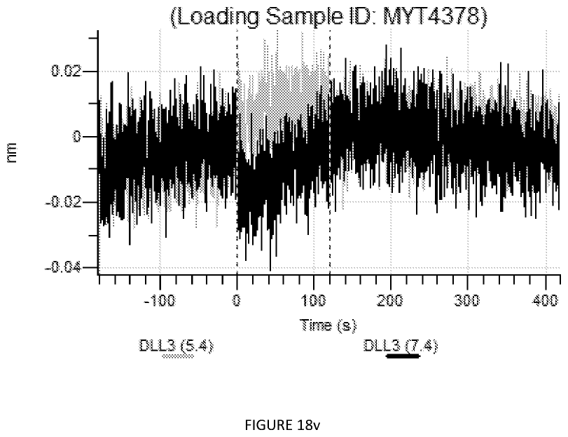

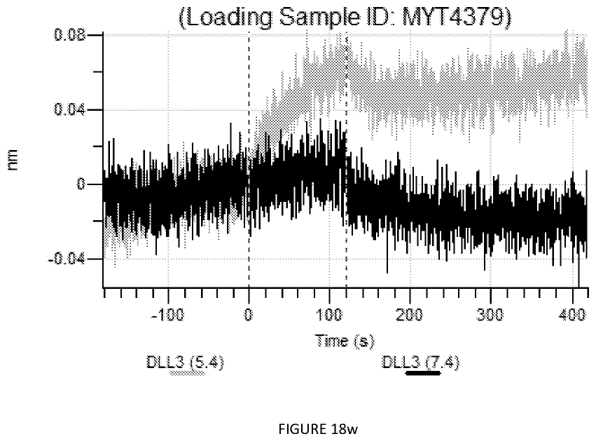

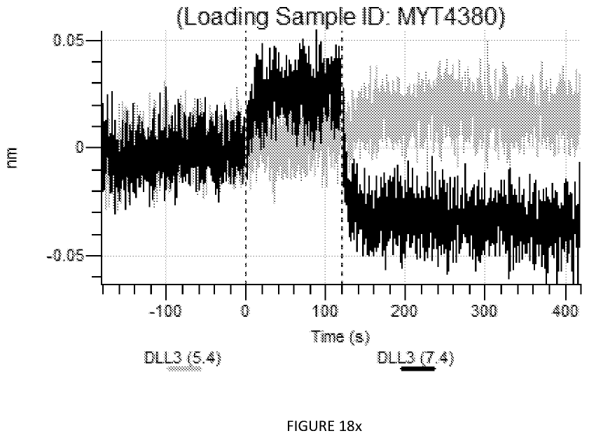

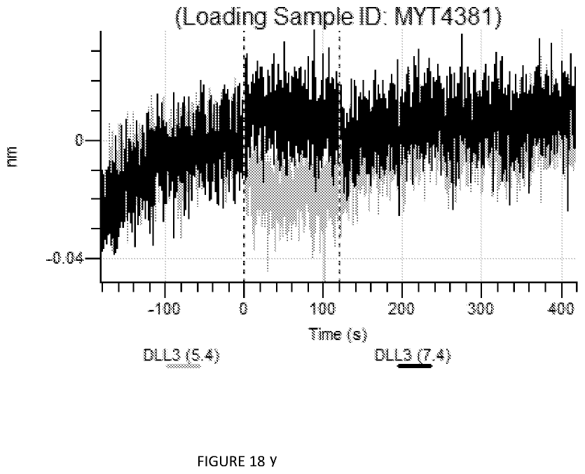

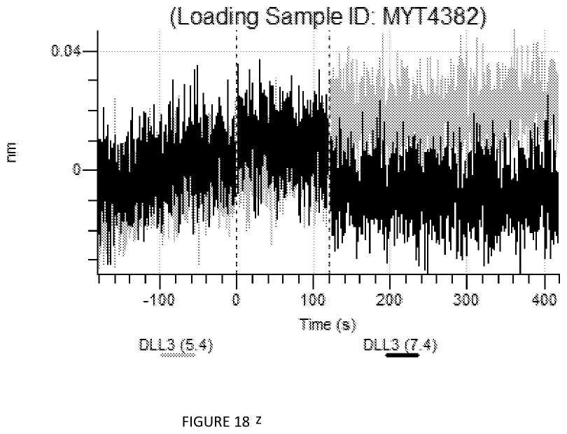

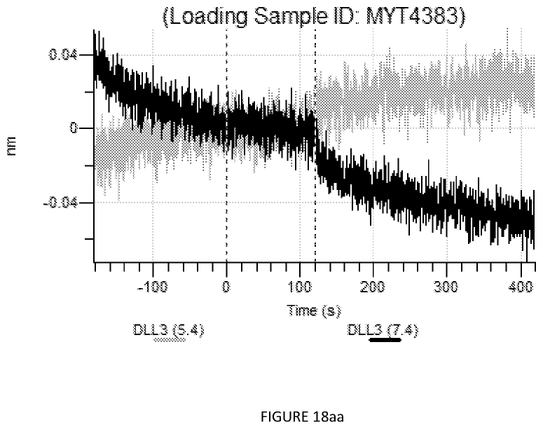

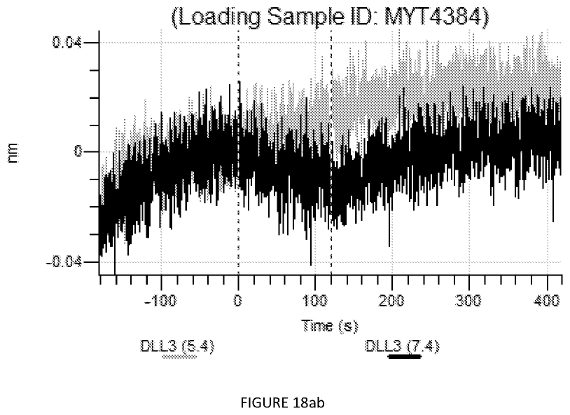

pH engineered anti-DLL3 variants were generated as VH-Fc fusions by transfection of Expi293 cells with a plasmid encoding for the variable heavy region fused genetically to human IgG1 Fc and as IgG variants by co-transfection of Expi293 cells with a) a heavy chain consisting of variable heavy chain region selected in Example 34 above and constant regions corresponding to human IgG1 isotype, and b) a light chain sequence previously known to pair with a heavy chain closest in sequence to the selected VH domain, using methods known to the art. After allowing for four days of protein expression, cell culture supernatants were collected, quantified by SDS-PAGE analysis (FIG. 17), and the pH dependence of the variant was evaluated using biolayer interferometry (BLI) on an Octet RED 384 or an Octet RED 96e instrument. Briefly, 5 μL of cell culture supernatant was diluted into 195 μL of 1×PBST, pH 7.4, for loading onto the sensor tips. Diluted supernatants were then captured on an anti-human Fc sensor (Forte Bio). A baseline was established using 1×PBST (50 mM Potassium Phosphate Buffer+150 mM NaCl+0.05% Tween 20), pH 7.4, and the sensor was associated with 100 nM of DLL3 (R&D Systems, Cat #9749-DL, Lot No. DFSG0719011) in 1×PBST, pH 7.4, for 120 sec to generate an association curve. In the dissociation phase, the antibody-antigen complex on the sensor was exposed to 1×PBST, pH 7.4, for 300-600 sec. Baseline, association, and dissociation were repeated using 1×PBST, pH 5.4, throughout in a separate condition. Association and dissociation phase curves were examined for each corresponding antibody variant at pH 5.4 and pH 7.4 to inform on two criteria: a) enhanced dissociation (e.g., higher koff values) at pH 5.4 as compared to other starting ABPCs of Examples 9-32, and b) reduced dissociation at pH 7.4 (e.g., lower koff values) compared to pH 5.4 in the antibody variant itself. VH-Fcs and reformatted IgG antibodies that showed either enhanced dissociation at pH 5.4 or reduced dissociation at pH 7.4 or both, as shown in FIG. 18 were selected for further analysis (e.g., MYT1047, MYT1048, MYT1050, MYT1053, MYT1054, MYT1055, MYT1056, MYT1057, MYT1060, MYT1061, MYT1062, MYT1063, MYT4373, MYT4374, and MYT4377).

### Example 36. Anti-DLL3 VH-Fc Epitope Binning

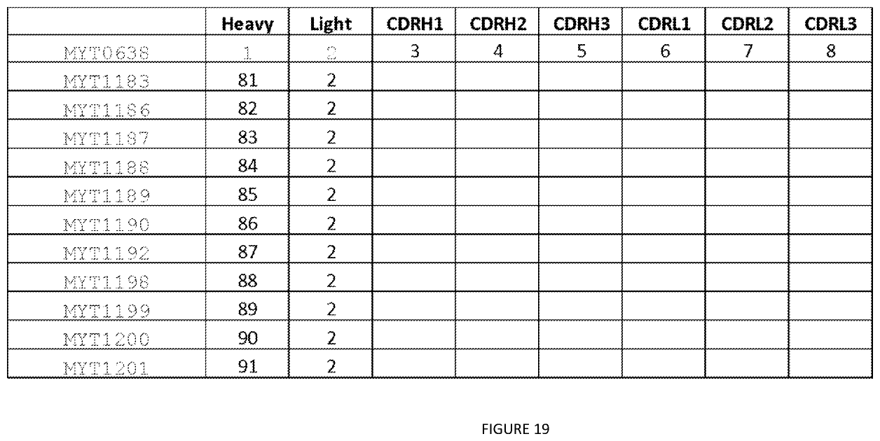

To identify VH-Fc molecules that bound to different epitopes of DLL3, a sandwich format competition binding assay was performed via Octet. Anti-Human capture (AHC) sensor tips were dipped into a solution of antibody with a known epitope (rovalpituzumab, SC16.4 or hSC16.13) and allowed to bind until saturation was achieved. The sensors were dipped back into 1×PBST, pH 7.4, buffer to achieve a stable baseline before 250 nM of recombinant human DLL3 (R&D Systems, Cat #9749-DL, Lot No. DFSG0719011) was bound to the sensor surface. Following a second baseline step in 1×PBST, pH 7.4, buffer the sensors were dipped into the VH-Fc cell culture supernatant. Additional binding was interpreted as binding to a separate epitope while a lack of binding was interpreted as the two molecules having some overlap in the epitope that they bind. A reduction in signal of 50% or more from the highest observed signal for a given VH-Fc molecule was classified as competition. As shown in FIG. 19, 8 out of 14 variants examined show competition with hSC16.13 (MYT1047, MYT1050, MYT1053, MYT1055, MYT1056, MYT1061, MYT1062, and MYT1063), three variants show competition with SC16.4 (MYT1054, MYT1056, and MYT1060), and two variants show competition with Rovalpituzumab (MYT1048 and MYT1058). In addition, two other variants did not show any competition with the three known tested antibodies (MYT1052 and MYT1057) confirming that they bind distinct epitopes.

### Example 37. Characterization of Binding Affinity for Anti-DLL3 mAbs

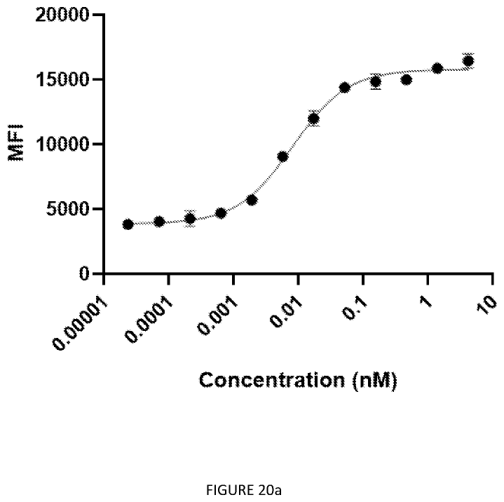

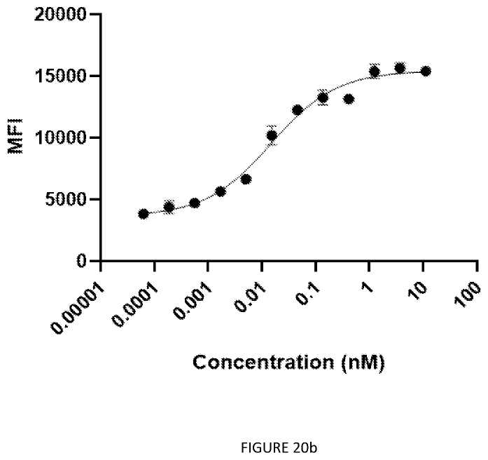

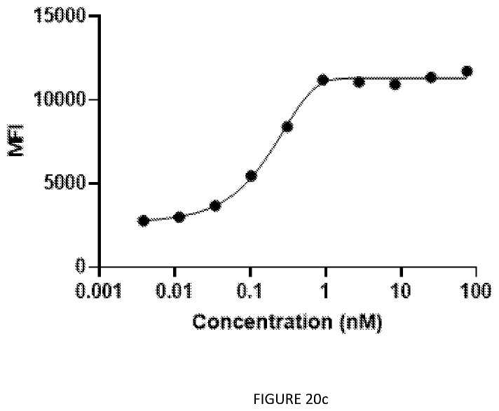

Binding affinity of selected anti-DLL3 pH engineered antibody variants from Examples 9-13 were measured on NCI-H82 cells. 100,000 cells per well were resuspended in 900 μL FC buffer in a 96 well deep well plate. Rovalpituzumab and pH engineered antibody variants were serially diluted at a 1:3 dilution. 100 μL of diluted antibody was added to each well with final concentration ranging from −100 nM to 0.024 μM and incubated for 2 hours at 4° C. Post incubation, the cells were spun at 2000 rpm for 2 min and supernatant was discarded. Cells were washed twice with 500 μL of ice-cold FC buffer and resuspended with 100 μL of FC buffer. The cells were then transferred from the deep well plate to 96 well round bottom plate, spun at 2000 rpm for 2 min and incubated with 100 μL of 10 μg/mL Alexa Fluor 488 conjugated goat anti-human IgG secondary antibody (ThermoFisher Scientific, A11013) for 30 min. Post incubation, cells were washed with FC buffer and resuspended in 100 μL of FC buffer to read on a BD Accuri C6 flow cytometer. Mean fluorescence intensity at each concentration per sample was background subtracted and plotted as shown in FIG. 20. Binding affinity was measured as a dissociation constant KD by GraphPad Prism assuming Michaelis-Menten binding kinetics. MYT1193 and MYT2829 show high affinity binding to cell surface DLL3 on NCI-H82 cells in the pM-nM range confirming that variants created through pH engineering can retain functionally appropriate affinities as compared to their corresponding starting ABPCs (e.g., rovalpituzumab). Variants with dissociation constants KD less than 100 nM were selected for further analysis.

### Example 38. Thermal Stability of Anti-DLL3 mAbs

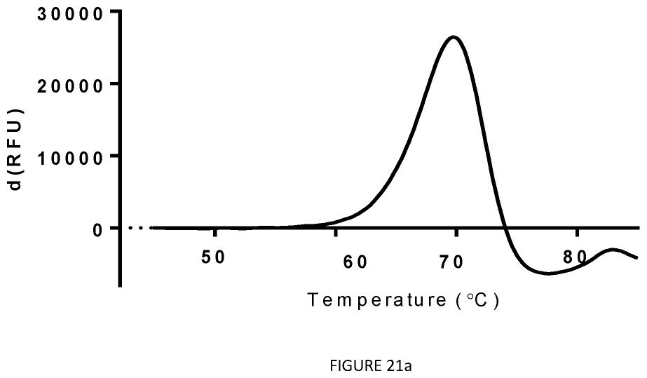

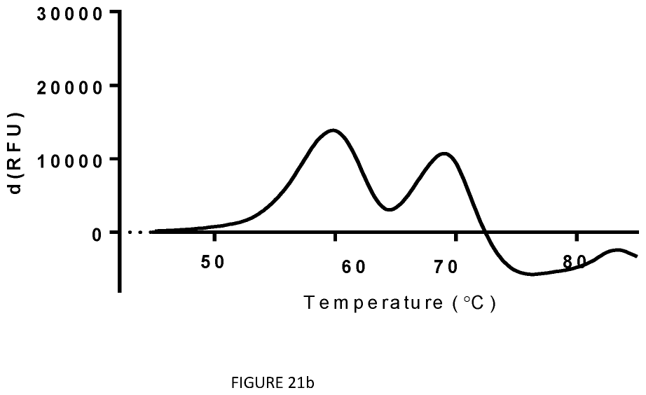

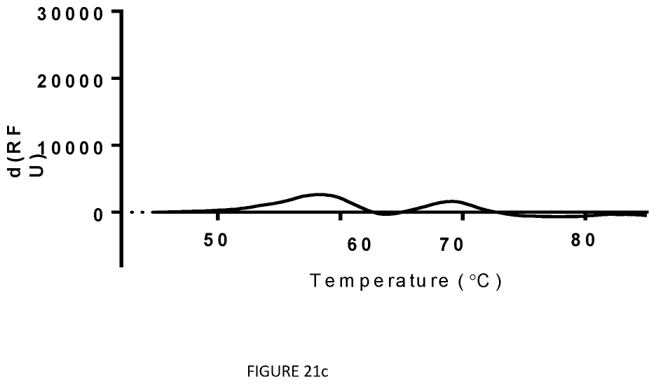

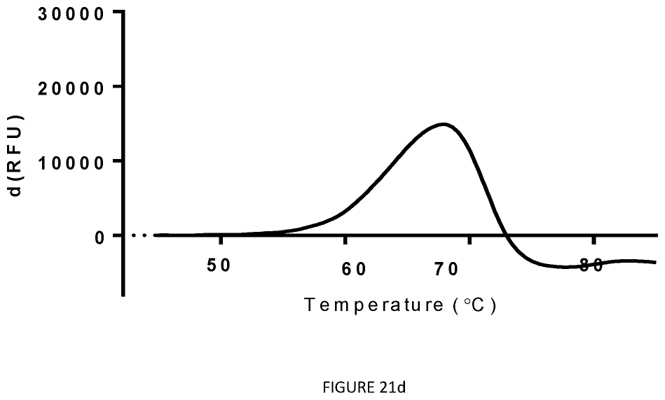

Protein melting temperature (Tm) was measured through the use of Differential Scanning Flourimetry (DSF). DSF visualizes protein unfolding by measuring the fluorescent signal from the molecule Sypro Orange (Thermo Scientific cat. no. S6650) as a protein unfolds due to heating. As a protein unfolds it exposes more hydrophilic regions to the Sypro Orange dye, which in turns binds to these hydrophilic regions resulting in increase in signal. The Tm for a protein is calculated as the half-maximal of the unfolding transition and can be visualized by plotting the first derivative of the Sypro Orange signal and finding a local maximum of this derivative plot. 20 μL of protein samples in 1×PBS, pH 7.4, was mixed with 5 μL of 25× Sypro Orange master mix, yielding a final concentration of 5× Sypro Orange. The samples were added to 96-well PCR plates (Thermo Scientific Cat. No. AB-2400/W) and sealed with optical covers (Thermo Scientific Cat. No. 4360954). The PCR plate was inserted into a real-time PCR machine (Thermo Scientific Quant Studio 3) and the plate temperature was stabilized for 3 minutes at 25° C. before ramping to 95° C. by 0.2° C. increments, stabilizing for 1 second before the Sypro Orange signal was measured. The melting temperature (Tm) values for anti-DLL3 variants are shown in FIG. 21. Several variants (MYT1185, MYT1193 and MYT2829) show similar melting temperature values to rovalpituzumab confirming that variants created through pH engineering can retain functionally appropriate thermal stabilities as compared to their corresponding starting ABPCs (e.g., rovalpituzumab). Variants (MYT1185, MYT1193 and MYT2829) with melting temperatures greater than the melting temperature of their corresponding starting ABPC (e.g., rovalpituzumab) minus 10° C. were selected for further analysis.

### Example 39. Specificity of Internalization and Endolysosomal Delivery for Anti-DLL3 mAbs

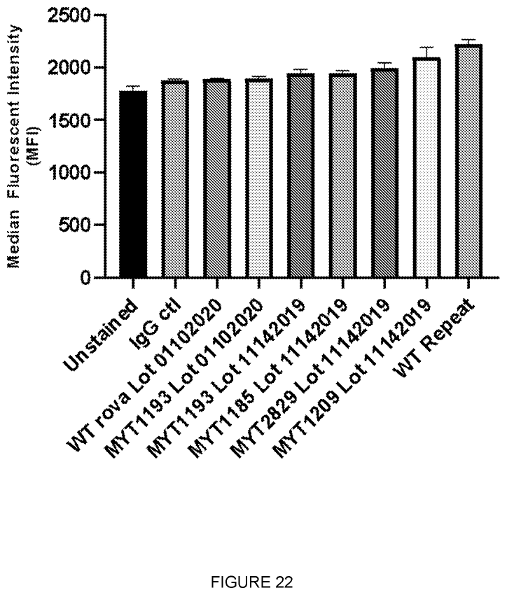

Specificity of anti-DLL3 mAbs for DLL3-mediated internalization and endolysosomal delivery into cells was evaluated by measuring the internalization and endolysosomal delivery in Raji cells (DLL3-). Briefly, cells were diluted to 2,000,000 cells/mL and 50 μL seeded into a 96-well flat bottom cell culture plate (Genesee Scientific; 25-109). Anti-DLL3 pH-engineered antibody variants, starting ABPC antibodies, control IgG1 isotype control (Cat #BP0297, Bioxcell), and vehicle control were diluted in native culture media to 100 nM and then mixed 1:1 with 300 nM Zenon pHrodo iFL Human IgG Labeling Reagent (ThermoFisher; Z25611). The mixture was incubated for 20 minutes at room temperature, followed by addition of 50 μL to cells. The mixture of cells, anti-DLL3 antibodies, and Zenon pHrodo iFL Human IgG Labeling Reagent was incubated at 37° C. 5% C02 for 24 hours. Following incubation, 100 μL of ice cold Flow Cytometry (FC) buffer (phosphate buffered saline (PBS) pH 7.4+2 mM ethylenediaminetetraacetic acid (EDTA)+2% (v/v) heat inactivated fetal bovine serum (HI FBS)) was added to each well. Cells were spun down at 4° C. for 2 min at 2000 rpm, washed with 200 μL ice cold FC buffer and resuspended in 100 μL ice cold FC buffer. Mean green fluorescence intensity was detected using a BD Accuri C6 flow cytometer. Data was analyzed using Flowjo analysis software. Internalization and endolysosomal delivery of anti-DLL3 wild type antibody (rovalpituzumab) and variants in non-specific Raji (DLL3-) cells is shown in FIG. 22. Rovalpituzumab and pH engineered variants (MYT1185, MYT1193 and MYT2829) show internalization and endolysosomal delivery similar to a human IgG1 isotype control confirming that pH engineering does not affect the specificity of target-dependent internalization and endolysosomal delivery of anti-DLL3 binding antibodies. Such pH engineered anti-DLL3 antibody variants with mean fluorescence intensity relative to their starting ABPC antibodies less than 2-fold increased as compared to IgG1 isotype control were selected for further analysis (MYT1185, MYT1193 and MYT2829). In this experiment, a drift was observed over time as samples were being read, which was able to be corrected for by reading identical samples treated with rovalpituzumab at the beginning and the end of the experiment.

The discoveries herein are in contrast to similar engineering on other antigens such as CLEC12a and two other targets wherein multiple variants per target showed enhanced dissociation at low pH, however, despite the favorable pH-dependent binding properties of these variants (which specifically bound CLEC12a and the two other targets), they had less than 10% increase in cell internalization and endolysosomal delivery as compared to the corresponding starting ABPC (e.g., the starting antibody). These variants (which specifically bound CLEC12a and the two other targets) also had similar biophysical characteristics (e.g., antibody expression, thermal stability, affinity at pH 7.4 etc.) to the corresponding starting ABPC (e.g., the starting antibody) confirming that this was not specific to the biophysical properties of the tested variant (i.e. the biophysical properties unrelated to enhanced dissociation at pH 5.4).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Sequence Appendix
>Heavy Chain of Rovalpituzumab IgG
(SEQ ID NO: 1)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Light Chain of Rovalpituzumab IgG
(SEQ ID NO: 2)
EIVMTQSPATLSVSPGERATLSCKASQSVSNDVVWYQQKPGQAPRLLIYYASNRYTGIP
ARFSGSGSGTEFTLTISSLQSEDFAVYYCQQDYTSPWTFGQGTKLEIK
>Heavy Chain CDR1 of Rovalpituzumab
(SEQ ID NO: 3)
KASGYTFTNYGMN
>Heavy Chain CDR2 of Rovalpituzumab
(SEQ ID NO: 4)
WINTYTGEPTYADDFKG
>Heavy Chain CDR3 of Rovalpituzumab
(SEQ ID NO: 5)
ARIGDSSPSDY
>Light Chain CDR1 of Rovalpituzumab
(SEQ ID NO: 6)
KASQSVSNDVV
>Light Chain CDR2 of Rovalpituzumab
(SEQ ID NO: 7)
YASNRYT
>Light Chain CDR3 of Rovalpituzumab
(SEQ ID NO: 8)
QQDYTSPW
>Mature Human DLL3
(SEQ ID NO: 9)
AGVFELQIHSFGPGPGPGAPRSPCSARLPCRLFFRVCLKPGLSEEAAESPCALGAALSARG
PVYTEQPGAPAPDLPLPDGLLQVPFRDAWPGTFSFIIETWREELGDQIGGPAWSLLARVA
GRRRLAAGGPWARDIQRAGAWELRFSYRARCEPPAVGTACTRLCRPRSAPSRCGPGLR
PCAPLEDECEAPLVCRAGCSPEHGFCEQPGECRCLEGWTGPLCTVPVSTSSCLSPRGPSS
ATTGCLVPGPGPCDGNPCANGGSCSETPRSFECTCPRGFYGLRCEVSGVTCADGPCFNG
GLCVGGADPDSAYICHCPPGFQGSNCEKRVDRCSLQPCRNGGLCLDLGHALRCRCRAG
FAGPRCEHDLDDCAGRACANGGTCVEGGGAHRCSCALGFGGRDCRERADPCAARPCA
HGGRCYAHFSGLVCACAPGYMGARCEFPVHPDGASALPAAPPGLRPGDPQRYLLPPAL
GLLVAAGVAGAALLLVHVRRRGHSQDAGSRLLAGTPEPSVHALPDALNNLRTQEGSGD
GPSSSVDWNRPEDVDPQGIYVISAPSIYAREVATPLFPPLHTGRAGQRQHLLFPYPSSILS
VK
>cDNA Encoding Mature Human DLL3
(SEQ ID NO: 10)
GCTGGCGTCTTCGAGCTGCAGATCCACTCTTTCGGGCCGGGTCCAGGCCCTGGGGCC
CCGCGGTCCCCCTGCAGCGCCCGGCTCCCCTGCCGCCTCTTCTTCAGAGTCTGCCTG
AAGCCTGGGCTCTCAGAGGAGGCCGCCGAGTCCCCGTGCGCCCTGGGCGCGGCGCT
GAGTGCGCGCGGACCGGTCTACACCGAGCAGCCCGGAGCGCCCGCGCCTGATCTCC
CACTGCCCGACGGCCTCTTGCAGGTGCCCTTCCGGGACGCCTGGCCTGGCACCTTCT
CTTTCATCATCGAAACCTGGAGAGAGGAGTTAGGAGACCAGATTGGAGGGCCCGCC
TGGAGCCTGCTGGCGCGCGTGGCTGGCAGGCGGCGCTTGGCAGCCGGAGGCCCGTG
GGCCCGGGACATTCAGCGCGCAGGCGCCTGGGAGCTGCGCTTCTCGTACCGCGCGC
GCTGCGAGCCGCCTGCCGTCGGGACCGCGTGCACGCGCCTCTGCCGTCCGCGCAGC
GCCCCCTCGCGGTGCGGTCCGGGACTGCGCCCCTGCGCACCGCTCGAGGACGAATG
TGAGGCGCCGCTGGTGTGCCGAGCAGGCTGCAGCCCTGAGCATGGCTTCTGTGAAC
AGCCCGGTGAATGCCGATGCCTAGAGGGCTGGACTGGACCCCTCTGCACGGTCCCT
GTCTCCACCAGCAGCTGCCTCAGCCCCAGGGGCCCGTCCTCTGCTACCACCGGATGC
CTTGTCCCTGGGCCTGGGCCCTGTGACGGGAACCCGTGTGCCAATGGAGGCAGCTGT
AGTGAGACACCCAGGTCCTTTGAATGCACCTGCCCGCGTGGGTTCTACGGGCTGCGG
TGTGAGGTGAGCGGGGTGACATGTGCAGATGGACCCTGCTTCAACGGCGGCTTGTGT
GTCGGGGGTGCAGACCCTGACTCTGCCTACATCTGCCACTGCCCACCCGGTTTCCAA
GGCTCCAACTGTGAGAAGAGGGTGGACCGGTGCAGCCTGCAGCCATGCCGCAATGG
CGGACTCTGCCTGGACCTGGGCCACGCCCTGCGCTGCCGCTGCCGCGCCGGCTTCGC
GGGTCCTCGCTGCGAGCACGACCTGGACGACTGCGCGGGCCGCGCCTGCGCTAACG
GCGGCACGTGTGTGGAGGGCGGCGGCGCGCACCGCTGCTCCTGCGCGCTGGGCTTC
GGCGGCCGCGACTGCCGCGAGCGCGCGGACCCGTGCGCCGCGCGCCCCTGTGCTCA
CGGCGGCCGCTGCTACGCCCACTTCTCCGGCCTCGTCTGCGCTTGCGCTCCCGGCTA
CATGGGAGCGCGGTGTGAGTTCCCAGTGCACCCCGACGGCGCAAGCGCCTTGCCCG
CGGCCCCGCCGGGCCTCAGGCCCGGGGACCCTCAGCGCTACCTTTTGCCTCCGGCTC
TGGGACTGCTCGTGGCCGCGGGCGTGGCCGGCGCTGCGCTCTTGCTGGTCCACGTGC
GCCGCCGTGGCCACTCCCAGGATGCTGGGTCTCGCTTGCTGGCTGGGACCCCGGAGC
CGTCAGTCCACGCACTCCCGGATGCACTCAACAACCTAAGGACGCAGGAGGGTTCC
GGGGATGGTCCGAGCTCGTCCGTAGATTGGAATCGCCCTGAAGATGTAGACCCTCA
AGGGATTTATGTCATATCTGCTCCTTCCATCTACGCTCGGGAGGTAGCGACGCCCCT
TTTCCCCCCGCTACACACTGGGCGCGCTGGGCAGAGGCAGCACCTGCTTTTTCCCTA
CCCTTCCTCGATTCTGTCCGTGAAA
>Extracellular Domain of DLL3
(SEQ ID NO: 11)
AGVFELQIHSFGPGPGPGAPRSPCSARLPCRLFFRVCLKPGLSEEAAESPCALGAALSARG
PVYTEQPGAPAPDLPLPDGLLQVPFRDAWPGTFSFIIETWREELGDQIGGPAWSLLARVA
GRRRLAAGGPWARDIQRAGAWELRFSYRARCEPPAVGTACTRLCRPRSAPSRCGPGLR
PCAPLEDECEAPLVCRAGCSPEHGFCEQPGECRCLEGWTGPLCTVPVSTSSCLSPRGPSS
ATTGCLVPGPGPCDGNPCANGGSCSETPRSFECTCPRGFYGLRCEVSGVTCADGPCFNG
GLCVGGADPDSAYICHCPPGFQGSNCEKRVDRCSLQPCRNGGLCLDLGHALRCRCRAG
FAGPRCEHDLDDCAGRACANGGTCVEGGGAHRCSCALGFGGRDCRERADPCAARPCA
HGGRCYAHF SGLVCACAPGYMGARCEFPVHPDGASALPAAPPGLRPGDPQRYL
>cDNA Encoding Extracellular Domain of DLL3
(SEQ ID NO: 12)
GCTGGCGTCTTCGAGCTGCAGATCCACTCTTTCGGGCCGGGTCCAGGCCCTGGGGCC
CCGCGGTCCCCCTGCAGCGCCCGGCTCCCCTGCCGCCTCTTCTTCAGAGTCTGCCTG
AAGCCTGGGCTCTCAGAGGAGGCCGCCGAGTCCCCGTGCGCCCTGGGCGCGGCGCT
GAGTGCGCGCGGACCGGTCTACACCGAGCAGCCCGGAGCGCCCGCGCCTGATCTCC
CACTGCCCGACGGCCTCTTGCAGGTGCCCTTCCGGGACGCCTGGCCTGGCACCTTCT
CTTTCATCATCGAAACCTGGAGAGAGGAGTTAGGAGACCAGATTGGAGGGCCCGCC
TGGAGCCTGCTGGCGCGCGTGGCTGGCAGGCGGCGCTTGGCAGCCGGAGGCCCGTG
GGCCCGGGACATTCAGCGCGCAGGCGCCTGGGAGCTGCGCTTCTCGTACCGCGCGC
GCTGCGAGCCGCCTGCCGTCGGGACCGCGTGCACGCGCCTCTGCCGTCCGCGCAGC
GCCCCCTCGCGGTGCGGTCCGGGACTGCGCCCCTGCGCACCGCTCGAGGACGAATG
TGAGGCGCCGCTGGTGTGCCGAGCAGGCTGCAGCCCTGAGCATGGCTTCTGTGAAC
AGCCCGGTGAATGCCGATGCCTAGAGGGCTGGACTGGACCCCTCTGCACGGTCCCT
GTCTCCACCAGCAGCTGCCTCAGCCCCAGGGGCCCGTCCTCTGCTACCACCGGATGC
CTTGTCCCTGGGCCTGGGCCCTGTGACGGGAACCCGTGTGCCAATGGAGGCAGCTGT
AGTGAGACACCCAGGTCCTTTGAATGCACCTGCCCGCGTGGGTTCTACGGGCTGCGG
TGTGAGGTGAGCGGGGTGACATGTGCAGATGGACCCTGCTTCAACGGCGGCTTGTGT
GTCGGGGGTGCAGACCCTGACTCTGCCTACATCTGCCACTGCCCACCCGGTTTCCAA
GGCTCCAACTGTGAGAAGAGGGTGGACCGGTGCAGCCTGCAGCCATGCCGCAATGG
CGGACTCTGCCTGGACCTGGGCCACGCCCTGCGCTGCCGCTGCCGCGCCGGCTTCGC
GGGTCCTCGCTGCGAGCACGACCTGGACGACTGCGCGGGCCGCGCCTGCGCTAACG
GCGGCACGTGTGTGGAGGGCGGCGGCGCGCACCGCTGCTCCTGCGCGCTGGGCTTC
GGCGGCCGCGACTGCCGCGAGCGCGCGGACCCGTGCGCCGCGCGCCCCTGTGCTCA
CGGCGGCCGCTGCTACGCCCACTTCTCCGGCCTCGTCTGCGCTTGCGCTCCCGGCTA
CATGGGAGCGCGGTGTGAGTTCCCAGTGCACCCCGACGGCGCAAGCGCCTTGCCCG
CGGCCCCGCCGGGCCTCAGGCCCGGGGACCCTCAGCGCTACCTT
>Heavy Chain of Rovalpituzumab IgG histidine scanning
variant #1
(SEQ ID NO: 13)
QVQLVQSGAEVKKPGASVKVSCHASGYTFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #2
(SEQ ID NO: 14)
QVQLVQSGAEVKKPGASVKVSCKHSGYTFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #3
(SEQ ID NO: 15)
QVQLVQSGAEVKKPGASVKVSCKAHGYTFTNYGMNWVRQAPGQGLEWMGWINTYT
GEPTYADDFKGRVTMTTDTSTSTAYMELRSLRSDDTAVYYCARIGDSSPSDYWGQGTL
VTVSS
>Heavy Chain of rovalpituzumab IgG histidine scanning
variant #4
(SEQ ID NO: 16)
QVQLVQSGAEVKKPGASVKVSCKASHYTFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #5
(SEQ ID NO: 17)
QVQLVQSGAEVKKPGASVKVSCKASGHTFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #6
(SEQ ID NO: 18)
QVQLVQSGAEVKKPGASVKVSCKASGYHFTNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #7
(SEQ ID NO: 19)
QVQLVQSGAEVKKPGASVKVSCKASGYTHTNYGMNWVRQAPGQGLEWMGWINTYT
GEPTYADDFKGRVTMTTDTSTSTAYMELRSLRSDDTAVYYCARIGDSSPSDYWGQGTL
VTVSS
>Heavy Chain of rovalpituzumab IgG histidine scanning
variant #8
(SEQ ID NO: 20)
QVQLVQSGAEVKKPGASVKVSCKASGYTFHNYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #9
(SEQ ID NO: 21)
QVQLVQSGAEVKKPGASVKVSCKASGYTFTHYGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #10
(SEQ ID NO: 22)
QVQLVQSGAEVKKPGASVKVSCKASGYTFTNHGMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #11
(SEQ ID NO: 23)
QVQLVQSGAEVKKPGASVKVSCKASGYTFTNYHMNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #12
(SEQ ID NO: 24)
QVQLVQSGAEVKKPGASVKVSCKASGYTFTNYGHNWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #13
(SEQ ID NO: 25)
QVQLVQSGAEVKKPGASVKVSCKASGYTFTNYGMEIWVRQAPGQGLEWMGWINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #14
(SEQ ID NO: 26)
QVQLVQSGAEVKKPGASVKVSCKASGYTFTNYGMNWVRQAPGQGLEWMGHINTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #15
(SEQ ID NO: 27)
QVQLVQSGAEVKKPGASVKVSCKASGYTFTNYGMNWVRQAPGQGLEWMGWHNTYT
GEPTYADDFKGRVTMTTDTSTSTAYMELRSLRSDDTAVYYCARIGDSSPSDYWGQGTL
VTVSS
>Heavy Chain of rovalpituzumab IgG histidine scanning
variant #16
(SEQ ID NO: 28)
QVQLVQSGAEVKKPGASVKVSCKASGYTFTNYGMNWVRQAPGQGLEWMGWIHTYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #17
(SEQ ID NO: 29)
QVQLVQSGAEVKKPGASVKVSCKASGYTFTNYGMNWVRQAPGQGLEWMGWINHYTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #18
(SEQ ID NO: 30)
QVQLVQSGAEVKKPGASVKVSCKASGYTFTNYGMNWVRQAPGQGLEWMGWINTHTG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #19
(SEQ ID NO: 31)
QVQLVQSGAEVKKPGASVKVSCKASGYTFTNYGMNWVRQAPGQGLEWMGWINTYHG
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #20
(SEQ ID NO: 32)
QVQLVQSGAEVKKPGASVKVSCKASGYTFTNYGMNWVRQAPGQGLEWMGWINTYTH
EPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #21
(SEQ ID NO: 33)
QVQLVQSGAEVKKPGASVKVSCKASGYTFTNYGMNWVRQAPGQGLEWMGWINTYTG
HPTYADDFKGRVTMTTDTSTSTAYMELRSLRSDDTAVYYCARIGDSSPSDYWGQGTLV
TVSS
>Heavy Chain of rovalpituzumab IgG histidine scanning
variant #22
(SEQ ID NO: 34)
QVQLVQSGAEVKKPGASVKVSCKASGYTFTNYGMNWVRQAPGQGLEWMGWINTYTG
EHTYADDFKGRVTMTTDTSTSTAYMELRSLRSDDTAVYYCARIGDSSPSDYWGQGTLV
TVSS
>Heavy Chain of rovalpituzumab IgG histidine scanning
variant #23
(SEQ ID NO: 35)
QVQLVQSGAEVKKPGASVKVSCKASGYTFTNYGMNWVRQAPGQGLEWMGWINTYTG
EPHYADDFKGRVTMTTDTSTSTAYMELRSLRSDDTAVYYCARIGDSSPSDYWGQGTLV
TVSS
>Heavy Chain of rovalpituzumab IgG histidine scanning
variant #24
(SEQ ID NO: 36)
QVQLVQSGAEVKKPGASVKVSCKASGYTFTNYGMNWVRQAPGQGLEWMGWINTYTG
EPTHADDFKGRVTMTTDTSTSTAYMELRSLRSDDTAVYYCARIGDSSPSDYWGQGTLV
TVSS
>Heavy Chain of rovalpituzumab IgG histidine scanning
variant #25
(SEQ ID NO: 37)
QVQLVQSGAEVKKPGASVKVSCKASGYTFTNYGMNWVRQAPGQGLEWMGWINTYTG
EPTYHDDFKGRVTMTTDTSTSTAYMELRSLRSDDTAVYYCARIGDSSPSDYWGQGTLV
TVSS
>Heavy Chain of rovalpituzumab IgG histidine scanning
variant #26
(SEQ ID NO: 38)
QVQLVQSGAEVKKPGASVKVSCKASGYTFTNYGMNWVRQAPGQGLEWMGWINTYTG
EPTYAHDFKGRVTMTTDTSTSTAYMELRSLRSDDTAVYYCARIGDSSPSDYWGQGTLV
TVSS
>Heavy Chain of rovalpituzumab IgG histidine scanning
variant #27
(SEQ ID NO: 39)
QVQLVQSGAEVKKPGASVKVSCKASGYTFTNYGMNWVRQAPGQGLEWMGWINTYTG
EPTYADHFKGRVTMTTDTSTSTAYMELRSLRSDDTAVYYCARIGDSSPSDYWGQGTLV
TVSS
>Heavy Chain of rovalpituzumab IgG histidine scanning
variant #28
(SEQ ID NO: 40)
QVQLVQSGAEVKKPGASVKVSCKASGYTFTNYGMNWVRQAPGQGLEWMGWINTYTG
EPTYADDHKGRVTMTTDTSTSTAYMELRSLRSDDTAVYYCARIGDSSPSDYWGQGTLV
TVSS
>Heavy Chain of rovalpituzumab IgG histidine scanning
variant #29
(SEQ ID NO: 41)
QVQLVQSGAEVKKPGASVKVSCKASGYTFTNYGMNWVRQAPGQGLEWMGWINTYTG
EPTYADDFHGRVTMTTDTSTSTAYMELRSLRSDDTAVYYCARIGDSSPSDYWGQGTLV
TVSS
>Heavy Chain of rovalpituzumab IgG histidine scanning
variant #30
(SEQ ID NO: 42)
QVQLVQSGAEVKKPGASVKVSCKASGYTFTNYGMNWVRQAPGQGLEWMGWINTYTG
EPTYADDFKHRVTMTTDTSTSTAYMELRSLRSDDTAVYYCARIGDSSPSDYWGQGTLV
TVSS
>Heavy Chain of rovalpituzumab IgG histidine scanning
variant #31
(SEQ ID NO: 43)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCHRIGDSSPSDYWGQGTLV
TVSS
>Heavy Chain of rovalpituzumab IgG histidine scanning
variant #32
(SEQ ID NO: 44)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCAHIGDSSPSDYWGQGTLV
TVSS
>Heavy Chain of rovalpituzumab IgG histidine scanning
variant #33
(SEQ ID NO: 45)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARHGDSSPSDYWGQGTLV
TVSS
>Heavy Chain of rovalpituzumab IgG histidine scanning
variant #34
(SEQ ID NO: 46)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIHDSSPSDYWGQGTLV
TVSS
>Heavy Chain of rovalpituzumab IgG histidine scanning
variant #35
(SEQ ID NO: 47)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGHSSPSDYWGQGTLV
TVSS
>Heavy Chain of rovalpituzumab IgG histidine scanning
variant #36
(SEQ ID NO: 48)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDHSPSDYWGQGTLV
TVSS
>Heavy Chain of rovalpituzumab IgG histidine scanning
variant #37
(SEQ ID NO: 49)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSHPSDYWGQGTLV
TVSS
>Heavy Chain of rovalpituzumab IgG histidine scanning
variant #38
(SEQ ID NO: 50)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSSHSDYWGQGTLV
TVSS
>Heavy Chain of rovalpituzumab IgG histidine scanning
variant #39
(SEQ ID NO: 51)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSSPHDYWGQGTLV
TVSS
>Heavy Chain of rovalpituzumab IgG histidine scanning
variant #40
(SEQ ID NO: 52)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSSPSHYWGQGTLV
TVSS
>Heavy Chain of rovalpituzumab IgG histidine scanning
variant #41
(SEQ ID NO: 53)
QVQLVQSGAEVKKPGASVKVSCKASGYTFTNYGMNWVRQAPGQGLEWMGWINTYTG
EPTYADDFKGRVTMTTDTSTSTAYMELRSLRSDDTAVYYCARIGDSSPSDHWGQGTLV
TVSS
>Light Chain of Rovalpituzumab IgG histidine scanning
variant #1
(SEQ ID NO: 54)
EIVMTQSPATLSVSPGERATLSCHASQSVSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #2
(SEQ ID NO: 55)
EIVMTQSPATLSVSPGERATLSCKHSQSVSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #3
(SEQ ID NO: 56)
EIVMTQSPATLSVSPGERATLSCKAHQSVSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #4
(SEQ ID NO: 57)
EIVMTQSPATLSVSPGERATLSCKASHSVSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #5
(SEQ ID NO: 58)
EIVMTQSPATLSVSPGERATLSCKASQHVSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #6
(SEQ ID NO: 59)
EIVMTQSPATLSVSPGERATLSCKASQSHSNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #7
(SEQ ID NO: 60)
EIVMTQSPATLSVSPGERATLSCKASQSVHNDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #8
(SEQ ID NO: 61)
EIVMTQSPATLSVSPGERATLSCKASQSVSHDVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #9
(SEQ ID NO: 62)
EIVMTQSPATLSVSPGERATLSCKASQSVSNHVVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #10
(SEQ ID NO: 63)
EIVMTQSPATLSVSPGERATLSCKASQSVSNDHVWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #11
(SEQ ID NO: 64)
EIVMTQSPATLSVSPGERATLSCKASQSVSNDVHWYQQKPGQAPRLLIYYASNRYTGIP
ARFSGSGSGTEFTLTISSLQSEDFAVYYCQQDYTSPWTFGQGTKLEIK
>Light Chain of Rovalpituzumab IgG histidine scanning
variant #12
(SEQ ID NO: 65)
EIVMTQSPATLSVSPGERATLSCKASQSVSNDVVWYQQKPGQAPRLLIYHASNRYTGIP
ARFSGSGSGTEFTLTISSLQSEDFAVYYCQQDYTSPWTFGQGTKLEIK
>Light Chain of Rovalpituzumab IgG histidine scanning
variant #13
(SEQ ID NO: 66)
EIVMTQSPATLSVSPGERATLSCKASQSVSNDVVWYQQKPGQAPRLLIYYHSNRYTGIP
ARFSGSGSGTEFTLTISSLQSEDFAVYYCQQDYTSPWTFGQGTKLEIK
>Light Chain of Rovalpituzumab IgG histidine scanning
variant #14
(SEQ ID NO: 67)
EIVMTQSPATLSVSPGERATLSCKASQSVSNDVVWYQQKPGQAPRLLIYYAHNRYTGIP
ARFSGSGSGTEFTLTISSLQSEDFAVYYCQQDYTSPWTFGQGTKLEIK
>Light Chain of Rovalpituzumab IgG histidine scanning
variant #15
(SEQ ID NO: 68)
EIVMTQSPATLSVSPGERATLSCKASQSVSNDVVWYQQKPGQAPRLLIYYASHRYTGIP
ARFSGSGSGTEFTLTISSLQSEDFAVYYCQQDYTSPWTFGQGTKLEIK
>Light Chain of Rovalpituzumab IgG histidine scanning
variant #16
(SEQ ID NO: 69)
EIVMTQSPATLSVSPGERATLSCKASQSVSNDVVWYQQKPGQAPRLLIYYASNHYTGIP
ARFSGSGSGTEFTLTISSLQSEDFAVYYCQQDYTSPWTFGQGTKLEIK
>Light Chain of Rovalpituzumab IgG histidine scanning
variant #17
(SEQ ID NO: 70)
EIVMTQSPATLSVSPGERATLSCKASQSVSNDVVWYQQKPGQAPRLLIYYASNRHTGIP
ARFSGSGSGTEFTLTISSLQSEDFAVYYCQQDYTSPWTFGQGTKLEIK
>Light Chain of Rovalpituzumab IgG histidine scanning
variant #18
(SEQ ID NO: 71)
EIVMTQSPATLSVSPGERATLSCKASQSVSNDVVWYQQKPGQAPRLLIYYASNRYHGIP
ARFSGSGSGTEFTLTISSLQSEDFAVYYCQQDYTSPWTFGQGTKLEIK
>Light Chain of Rovalpituzumab IgG histidine scanning
variant #19
(SEQ ID NO: 72)
EIVMTQSPATLSVSPGERATLSCKASQSVSNDVVWYQQKPGQAPRLLIYYASNRYTGIP
ARFSGSGSGTEFTLTISSLQSEDFAVYYCHQDYTSPWTFGQGTKLEIK
>Light Chain of Rovalpituzumab IgG histidine scanning
variant #20
(SEQ ID NO: 73
EIVMTQSPATLSVSPGERATLSCKASQSVSNDVVWYQQKPGQAPRLLIYYASNRYTGIP
ARFSGSGSGTEFTLTISSLQSEDFAVYYCQHDYTSPWTFGQGTKLEIK
>Light Chain of Rovalpituzumab IgG histidine scanning
variant #21
(SEQ ID NO: 74)
EIVMTQSPATLSVSPGERATLSCKASQSVSNDVVWYQQKPGQAPRLLIYYASNRYTGIP
ARFSGSGSGTEFTLTISSLQSEDFAVYYCQQHYTSPWTFGQGTKLEIK
>Light Chain of Rovalpituzumab IgG histidine scanning
variant #22
(SEQ ID NO: 75)
EIVMTQSPATLSVSPGERATLSCKASQSVSNDVVWYQQKPGQAPRLLIYYASNRYTGIP
ARFSGSGSGTEFTLTISSLQSEDFAVYYCQQDHTSPWTFGQGTKLEIK
>Light Chain of Rovalpituzumab IgG histidine scanning
variant #23
(SEQ ID NO: 76)
EIVMTQSPATLSVSPGERATLSCKASQSVSNDVVWYQQKPGQAPRLLIYYASNRYTGIP
ARFSGSGSGTEFTLTISSLQSEDFAVYYCQQDYHSPWTFGQGTKLEIK
>Light Chain of Rovalpituzumab IgG histidine scanning
variant #24
(SEQ ID NO: 77)
EIVMTQSPATLSVSPGERATLSCKASQSVSNDVVWYQQKPGQAPRLLIYYASNRYTGIP
ARFSGSGSGTEFTLTISSLQSEDFAVYYCQQDYTHPWTFGQGTKLEIK
>Light Chain of Rovalpituzumab IgG histidine scanning
variant #25
(SEQ ID NO: 78)
EIVMTQSPATLSVSPGERATLSCKASQSVSNDVVWYQQKPGQAPRLLIYYASNRYTGIP
ARFSGSGSGTEFTLTISSLQSEDFAVYYCQQDYTSHWTFGQGTKLEIK
>Light Chain of Rovalpituzumab IgG histidine scanning
variant #26
(SEQ ID NO: 79)
EIVMTQSPATLSVSPGERATLSCKASQSVSNDVVWYQQKPGQAPRLLIYYASNRYTGIP
ARFSGSGSGTEFTLTISSLQSEDFAVYYCQQDYTSPHTFGQGTKLEIK
>Light Chain of Rovalpituzumab IgG histidine scanning
variant #27
(SEQ ID NO: 80)
EIVMTQSPATLSVSPGERATLSCKASQSVSNDVVWYQQKPGQAPRLLIYYASNRYTGIP
ARFSGSGSGTEFTLTISSLQSEDFAVYYCQQDYTSPWHFGQGTKLEIK
>Heavy Chain Combinations of Rovalpituzumab IgG histidine
scanning variant #1
(SEQ ID NO: 81)
QVQLVQSGAEVKKPGASVKVSCKASGYTHTNYGMNWVRQAPGQGLEWMGWINTHTGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #2
(SEQ ID NO: 82)
QVQLVQSGAEVKKPGASVKVSCKASGYTFTHYGMNWVRQAPGQGLEWMGWINTHTGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #3
(SEQ ID NO: 83)
QVQLVQSGAEVKKPGASVKVSCKASGYTFTHYGMNWVRQAPGQGLEWMGWINTYHGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #4
(SEQ ID NO: 84)
QVQLVQSGAEVKKPGASVKVSCKASGYTFTHYGMNWVRQAPGQGLEWMGWINTYTGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #5
(SEQ ID NO: 85)
QVQLVQSGAEVKKPGASVKVSCKASGYTFTNYGMNWVRQAPGQGLEWMGWINTHHGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #6
(SEQ ID NO: 86)
QVQLVQSGAEVKKPGASVKVSCKASGYTFTNYGMNWVRQAPGQGLEWMGWINTHTGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #7
(SEQ ID NO: 87)
QVQLVQSGAEVKKPGASVKVSCKASGYTHTHYGMNWVRQAPGQGLEWMGWINTHTGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #8
(SEQ ID NO: 88)
QVQLVQSGAEVKKPGASVKVSCKASGYTFTHYGMNWVRQAPGQGLEWMGWINTHHGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #9
(SEQ ID NO: 89)
QVQLVQSGAEVKKPGASVKVSCKASGYTFTHYGMNWVRQAPGQGLEWMGWINTHTGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #10
(SEQ ID NO: 90)
QVQLVQSGAEVKKPGASVKVSCKASGYTFTHYGMNWVRQAPGQGLEWMGWINTYHGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #11
(SEQ ID NO: 91)
QVQLVQSGAEVKKPGASVKVSCKASGYTFTNYGMNWVRQAPGQGLEWMGWINTHHGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #12
(SEQ ID NO: 92)
QVQLVQSGAEVKKPGASVKVSCKASGYTHTHYGMNWVRQAPGQGLEWMGWINTYTGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #13
(SEQ ID NO: 93)
QVQLVQSGAEVKKPGASVKVSCKASGYTHTNYGMNWVRQAPGQGLEWMGWINTYTGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #14
(SEQ ID NO: 94)
QVQLVQSGAEVKKPGASVKVSCKASGYTFTNYGMNWVRQAPGQGLEWMGWINTYHGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #15
(SEQ ID NO: 95)
QVQLVQSGAEVKKPGASVKVSCKASGYTHTHYGMNWVRQAPGQGLEWMGWINTYHGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #16
(SEQ ID NO: 96)
QVQLVQSGAEVKKPGASVKVSCKASGYTHTHYGMNWVRQAPGQGLEWMGWINTYTGEPTYA
DDFKGRVTMTTDTSTSTAYMELRSLRSDDTAVYYCARIGDSSPHDYWGQGTLVTVSS
>Heavy Chain Combinations of Rovalpituzumab IgG histidine
scanning variant #17
(SEQ ID NO: 97)
QVQLVQSGAEVKKPGASVKVSCKASGYTHTNYGMNWVRQAPGQGLEWMGWINTHHGEPTYA
DDFKGRVTMTTDTSTSTAYMELRSLRSDDTAVYYCARIGDSSPSDYWGQGTLVTVSS
>Heavy Chain Combinations of Rovalpituzumab IgG histidine
scanning variant #18
(SEQ ID NO: 98)
QVQLVQSGAEVKKPGASVKVSCKASGYTHTNYGMNWVRQAPGQGLEWMGWINTHTGEPTYA
DDFKGRVTMTTDTSTSTAYMELRSLRSDDTAVYYCARIGDSSPHDYWGQGTLVTVSS
>Light Chain Combinations of Rovalpituzumab IgG histidine
scanning variant #1
(SEQ ID NO: 99)
EIVMTQSPATLSVSPGERATLSCKAHQSVSNDVVWYQQKPGQAPRLLIYHASNRYTGIPARFSGS
GSGTEFTLTISSLQSEDFAVYYCQQDYTSPWTFGQGTKLEIK
>Light Chain Combinations of Rovalpituzumab IgG histidine
scanning variant #2
(SEQ ID NO: 100)
EIVMTQSPATLSVSPGERATLSCKAHQSVSNDVVWYQQKPGQAPRLLIYYASNRYTGIPARFSGS
GSGTEFTLTISSLQSEDFAVYYCQQDYHSPWTFGQGTKLEIK
>Light Chain Combinations of Rovalpituzumab IgG histidine
scanning variant #3
(SEQ ID NO: 101)
EIVMTQSPATLSVSPGERATLSCKAHQSVSNDVVWYQQKPGQAPRLLIYYASNRYTGIPARFSGS
GSGTEFTLTISSLQSEDFAVYYCQQDYTHPWTFGQGTKLEIK
>Light Chain Combinations of Rovalpituzumab IgG histidine
scanning variant #4
(SEQ ID NO: 102)
EIVMTQSPATLSVSPGERATLSCKAHQSVSNDVVWYQQKPGQAPRLLIYYASNRYTGIPARFSGS
GSGTEFTLTISSLQSEDFAVYYCQQDYTSPWHFGQGTKLEIK
>Light Chain Combinations of Rovalpituzumab IgG histidine
scanning variant #5
(SEQ ID NO: 103)
EIVMTQSPATLSVSPGERATLSCKASQSVSNDVVWYQQKPGQAPRLLIYHASNRYTGIPARFSGS
GSGTEFTLTISSLQSEDFAVYYCQQDYHSPWTFGQGTKLEIK
>Light Chain Combinations of Rovalpituzumab IgG histidine
scanning variant #6
(SEQ ID NO: 104)
EIVMTQSPATLSVSPGERATLSCKASQSVSNDVVWYQQKPGQAPRLLIYHASNRYTGIPARFSGS
GSGTEFTLTISSLQSEDFAVYYCQQDYTHPWTFGQGTKLEIK
>Light Chain Combinations of Rovalpituzumab IgG histidine
scanning variant #7
(SEQ ID NO: 105)
EIVMTQSPATLSVSPGERATLSCKASQSVSNDVVWYQQKPGQAPRLLIYHASNRYTGIPARFSGS
GSGTEFTLTISSLQSEDFAVYYCQQDYTSPWHFGQGTKLEIK
>Light Chain Combinations of Rovalpituzumab IgG histidine
scanning variant #8
(SEQ ID NO: 106)
EIVMTQSPATLSVSPGERATLSCKASQSVSNDVVWYQQKPGQAPRLLIYYASNRYTGIPARFSGS
GSGTEFTLTISSLQSEDFAVYYCQQDYFIHPWTFGQGTKLEIK
>Light Chain Combinations of Rovalpituzumab IgG histidine
scanning variant #9
(SEQ ID NO: 107)
EIVMTQSPATLSVSPGERATLSCKASQSVSNDVVWYQQKPGQAPRLLIYYASNRYTGIPARFSGS
GSGTEFTLTISSLQSEDFAVYYCQQDYHSPWHFGQGTKLEIK
>Light Chain Combinations of Rovalpituzumab IgG histidine
scanning variant #10
(SEQ ID NO: 108)
EIVMTQSPATLSVSPGERATLSCKASQSVSNDVVWYQQKPGQAPRLLIYYASNRYTGIPARFSGS
GSGTEFTLTISSLQSEDFAVYYCQQDYTHPWHFGQGTKLEIK
>Light Chain Combinations of Rovalpituzumab IgG histidine
scanning variant #11
(SEQ ID NO: 109)
EIVMTQSPATLSVSPGERATLSCKAHQSVSNDVVWYQQKPGQAPRLLIYHASNRYTGIPARFSGS
GSGTEFTLTISSLQSEDFAVYYCQQDYHSPWTFGQGTKLEIK
>Light Chain Combinations of Rovalpituzumab IgG histidine
scanning variant #12
(SEQ ID NO: 110)
EIVMTQSPATLSVSPGERATLSCKAHQSVSNDVVWYQQKPGQAPRLLIYHASNRYTGIPARFSGS
GSGTEFTLTISSLQSEDFAVYYCQQDYTHPWTFGQGTKLEIK
>Light Chain Combinations of Rovalpituzumab IgG histidine
scanning variant #13
(SEQ ID NO: 111)
EIVMTQSPATLSVSPGERATLSCKAHQSVSNDVVWYQQKPGQAPRLLIYHASNRYTGIPARFSGS
GSGTEFTLTISSLQSEDFAVYYCQQDYTSPWHFGQGTKLEIK
>Light Chain Combinations of Rovalpituzumab IgG histidine
scanning variant #14
(SEQ ID NO: 112)
EIVMTQSPATLSVSPGERATLSCKAHQSVSNDVVWYQQKPGQAPRLLIYYASNRYTGIPARFSGS
GSGTEFTLTISSLQSEDFAVYYCQQDYFIHPWTFGQGTKLEIK
>Light Chain Combinations of Rovalpituzumab IgG histidine
scanning variant #15
(SEQ ID NO: 113)
EIVMTQSPATLSVSPGERATLSCKAHQSVSNDVVWYQQKPGQAPRLLIYYASNRYTGIPARFSGS
GSGTEFTLTISSLQSEDFAVYYCQQDYHSPWHFGQGTKLEIK
>Light Chain Combinations of Rovalpituzumab IgG histidine
scanning variant #16
(SEQ ID NO: 114)
EIVMTQSPATLSVSPGERATLSCKAHQSVSNDVVWYQQKPGQAPRLLIYYASNRYTGIPARFSGS
GSGTEFTLTISSLQSEDFAVYYCQQDYTHPWHFGQGTKLEIK
>Light Chain Combinations of Rovalpituzumab IgG histidine
scanning variant #17
(SEQ ID NO: 115)
EIVMTQSPATLSVSPGERATLSCKASQSVSNDVVWYQQKPGQAPRLLIYHASNRYTGIPARFSGS
GSGTEFTLTISSLQSEDFAVYYCQQDYFIHPWTFGQGTKLEIK
>Light Chain Combinations of Rovalpituzumab IgG histidine
scanning variant #18
(SEQ ID NO: 116)
EIVMTQSPATLSVSPGERATLSCKASQSVSNDVVWYQQKPGQAPRLLIYHASNRYTGIPARFSGS
GSGTEFTLTISSLQSEDFAVYYCQQDYHSPWHFGQGTKLEIK
>Light Chain Combinations of Rovalpituzumab IgG histidine
scanning variant #19
(SEQ ID NO: 117)
EIVMTQSPATLSVSPGERATLSCKASQSVSNDVVWYQQKPGQAPRLLIYHASNRYTGIPARFSGS
GSGTEFTLTISSLQSEDFAVYYCQQDYTHPWHFGQGTKLEIK
>Light Chain Combinations of Rovalpituzumab IgG histidine
scanning variant #20
(SEQ ID NO: 118)
EIVMTQSPATLSVSPGERATLSCKASQSVSNDVVWYQQKPGQAPRLLIYYASNRYTGIPARFSGS
GSGTEFTLTISSLQSEDFAVYYCQQDYFIHPWHFGQGTKLEIK
>Heavy Chain of SC16.4 IgG
(SEQ ID NO: 119)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGYAMDYWGQGTSVTVSS
>Light Chain of SC16.4 IgG
(SEQ ID NO: 120)
DIQMTQTTSSLSASLGDRVTISCRASQDISNYLNWYQQKPDGTVKLLIYYTSRLHSGVPSRFSGSG
SGTDYSLTISNLELEDIATYFCQQGDMLPWTFGGGTKLEIK
>Heavy Chain of SC16.4 IgG histidine scanning variant #1
(SEQ ID NO: 121)
QIQLVQSGPELKKPGETVKISCKASHYTFTDYSMHWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #2
(SEQ ID NO: 122)
QIQLVQSGPELKKPGETVKISCKASGHTFTDYSMHWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #3
(SEQ ID NO: 123)
QIQLVQSGPELKKPGETVKISCKASGYHFTDYSMHWVKQAPGKGLKWMGWINTETGEPGYAD
DFKGRFAFSLETSASTAYLQINNLKNEDTATYFCARYDGYAMDYWGQGTSVTVSS
>Heavy Chain of SC16.4 IgG histidine scanning variant #4
(SEQ ID NO: 124)
QIQLVQSGPELKKPGETVKISCKASGYTHTDYSMHWVKQAPGKGLKWMGWINTETGEPGYAD
DFKGRFAFSLETSASTAYLQINNLKNEDTATYFCARYDGYAMDYWGQGTSVTVSS
>Heavy Chain of SC16.4 IgG histidine scanning variant #5
(SEQ ID NO: 125)
QIQLVQSGPELKKPGETVKISCKASGYTFHDYSMHWVKQAPGKGLKWMGWINTETGEPGYAD
DFKGRFAFSLETSASTAYLQINNLKNEDTATYFCARYDGYAMDYWGQGTSVTVSS
>Heavy Chain of SC16.4 IgG histidine scanning variant #6
(SEQ ID NO: 126)
QIQLVQSGPELKKPGETVKISCKASGYTFTHYSMHWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #7
(SEQ ID NO: 127)
QIQLVQSGPELKKPGETVKISCKASGYTFTDHSMHWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #8
(SEQ ID NO: 128)
QIQLVQSGPELKKPGETVKISCKASGYTFTDYHMHWVKQAPGKGLKWMGWINTETGEPGYAD
DFKGRFAFSLETSASTAYLQINNLKNEDTATYFCARYDGYAMDYWGQGTSVTVSS
>Heavy Chain of SC16.4 IgG histidine scanning variant #9
(SEQ ID NO: 129)
QIQLVQSGPELKKPGETVKISCKASGYTFTDYSHHWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #10
(SEQ ID NO: 130)
QIQLVQSGPELKKPGETVKISCKASGYTFTDYSMAWVKQAPGKGLKWMGWINTETGEPGYADD
FKGRFAFSLETSASTAYLQINNLKNEDTATYFCARYDGYAMDYWGQGTSVTVSS
>Heavy Chain of SC16.4 IgG histidine scanning variant #11
(SEQ ID NO: 131)
QIQLVQSGPELKKPGETVKISCKASGYTFTDYSMHWVKQAPGKGLKWMGHINTETGEPGYADD
FKGRFAFSLETSASTAYLQINNLKNEDTATYFCARYDGYAMDYWGQGTSVTVSS
>Heavy Chain of SC16.4 IgG histidine scanning variant #12
(SEQ ID NO: 132)
QIQLVQSGPELKKPGETVKISCKASGYTFTDYSMHWVKQAPGKGLKWMGWHNTETGEPGYAD
DFKGRFAFSLETSASTAYLQINNLKNEDTATYFCARYDGYAMDYWGQGTSVTVSS
>Heavy Chain of SC16.4 IgG histidine scanning variant #13
(SEQ ID NO: 133)
QIQLVQSGPELKKPGETVKISCKASGYTFTDYSMHWVKQAPGKGLKWMGWIHTETGEPGYADD
FKGRFAFSLETSASTAYLQINNLKNEDTATYFCARYDGYAMDYWGQGTSVTVSS
>Heavy Chain of SC16.4 IgG histidine scanning variant #14
(SEQ ID NO: 134)
QIQLVQSGPELKKPGETVKISCKASGYTFTDYSMHWVKQAPGKGLKWMGWINHETGEPGYAD
DFKGRFAFSLETSASTAYLQINNLKNEDTATYFCARYDGYAMDYWGQGTSVTVSS
>Heavy Chain of SC16.4 IgG histidine scanning variant #15
(SEQ ID NO: 135)
QIQLVQSGPELKKPGETVKISCKASGYTFTDYSMHWVKQAPGKGLKWMGWINTHTGEPGYAD
DFKGRFAFSLETSASTAYLQINNLKNEDTATYFCARYDGYAMDYWGQGTSVTVSS
>Heavy Chain of SC16.4 IgG histidine scanning variant #16
(SEQ ID NO: 136)
QIQLVQSGPELKKPGETVKISCKASGYTFTDYSMHWVKQAPGKGLKWMGWINTEHGEPGYAD
DFKGRFAFSLETSASTAYLQINNLKNEDTATYFCARYDGYAMDYWGQGTSVTVSS
>Heavy Chain of SC16.4 IgG histidine scanning variant #17
(SEQ ID NO: 137)
QIQLVQSGPELKKPGETVKISCKASGYTFTDYSMHWVKQAPGKGLKWMGWINTETHEPGYADD
FKGRFAFSLETSASTAYLQINNLKNEDTATYFCARYDGYAMDYWGQGTSVTVSS
>Heavy Chain of SC16.4 IgG histidine scanning variant #18
(SEQ ID NO: 138)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARYDGYAMDYWGQGTSVTVSS
>Heavy Chain of SC16.4 IgG histidine scanning variant #19
(SEQ ID NO: 139)
QIQLVQSGPELKKPGETVKISCKASGYTFTDYSMHWVKQAPGKGLKWMGWINTETGEHGYAD
DFKGRFAFSLETSASTAYLQINNLKNEDTATYFCARYDGYAMDYWGQGTSVTVSS
>Heavy Chain of SC16.4 IgG histidine scanning variant #20
(SEQ ID NO: 140)
QIQLVQSGPELKKPGETVKISCKASGYTFTDYSMHWVKQAPGKGLKWMGWINTETGEPHYADD
FKGRFAFSLETSASTAYLQINNLKNEDTATYFCARYDGYAMDYWGQGTSVTVSS
>Heavy Chain of SC16.4 IgG histidine scanning variant #21
(SEQ ID NO: 141)
QIQLVQSGPELKKPGETVKISCKASGYTFTDYSMHWVKQAPGKGLKWMGWINTETGEPGHADD
FKGRFAFSLETSASTAYLQINNLKNEDTATYFCARYDGYAMDYWGQGTSVTVSS
>Heavy Chain of SC16.4 IgG histidine scanning variant #22
(SEQ ID NO: 142)
QIQLVQSGPELKKPGETVKISCKASGYTFTDYSMHWVKQAPGKGLKWMGWINTETGEPGYHDD
FKGRFAFSLETSASTAYLQINNLKNEDTATYFCARYDGYAMDYWGQGTSVTVSS
>Heavy Chain of SC16.4 IgG histidine scanning variant #23
(SEQ ID NO: 143)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARYDGYAMDYWGQGTSVTVSS
>Heavy Chain of SC16.4 IgG histidine scanning variant #24
(SEQ ID NO: 144)
QIQLVQSGPELKKPGETVKISCKASGYTFTDYSMHWVKQAPGKGLKWMGWINTETGEPGYADH
FKGRFAFSLETSASTAYLQINNLKNEDTATYFCARYDGYAMDYWGQGTSVTVSS
>Heavy Chain of SC16.4 IgG histidine scanning variant #25
(SEQ ID NO: 145)
QIQLVQSGPELKKPGETVKISCKASGYTFTDYSMHWVKQAPGKGLKWMGWINTETGEPGYADD
HKGRFAFSLETSASTAYLQINNLKNEDTATYFCARYDGYAMDYWGQGTSVTVSS
>Heavy Chain of SC16.4 IgG histidine scanning variant #26
(SEQ ID NO: 146)
QIQLVQSGPELKKPGETVKISCKASGYTFTDYSMHWVKQAPGKGLKWMGWINTETGEPGYADD
FHGRFAFSLETSASTAYLQINNLKNEDTATYFCARYDGYAMDYWGQGTSVTVSS
>Heavy Chain of SC16.4 IgG histidine scanning variant #27
(SEQ ID NO: 147)
QIQLVQSGPELKKPGETVKISCKASGYTFTDYSMHWVKQAPGKGLKWMGWINTETGEPGYADD
FKHRFAFSLETSASTAYLQINNLKNEDTATYFCARYDGYAMDYWGQGTSVTVSS
>Heavy Chain of SC16.4 IgG histidine scanning variant #28
(SEQ ID NO: 148)
QIQLVQSGPELKKPGETVKISCKASGYTFTDYSMHWVKQAPGKGLKWMGWINTETGEPGYADD
FKGRFAFSLETSASTAYLQINNLKNEDTATYFCHRYDGYAMDYWGQGTSVTVSS
>Heavy Chain of SC16.4 IgG histidine scanning variant #29
(SEQ ID NO: 149)
QIQLVQSGPELKKPGETVKISCKASGYTFTDYSMHWVKQAPGKGLKWMGWINTETGEPGYADD
FKGRFAFSLETSASTAYLQINNLKNEDTATYFCAHYDGYAMDYWGQGTSVTVSS
>Heavy Chain of SC16.4 IgG histidine scanning variant #30
(SEQ ID NO: 150)
QIQLVQSGPELKKPGETVKISCKASGYTFTDYSMHWVKQAPGKGLKWMGWINTETGEPGYADD
FKGRFAFSLETSASTAYLQINNLKNEDTATYFCARHDGYAMDYWGQGTSVTVSS
>Heavy Chain of SC16.4 IgG histidine scanning variant #31
(SEQ ID NO: 151)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYHGYAMDYWGQGTSVTVSS
>Heavy Chain of SC16.4 IgG histidine scanning variant #32
(SEQ ID NO: 152)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDHYAMDYWGQGTSVTVSS
>Heavy Chain of SC16.4 IgG histidine scanning variant #33
(SEQ ID NO: 153)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGHAMDYWGQGTSVTVSS
>Heavy Chain of SC16.4 IgG histidine scanning variant #34
(SEQ ID NO: 154)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGYHMDYWGQGTSVTVSS
>Heavy Chain of SC16.4 IgG histidine scanning variant #35
(SEQ ID NO: 155)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGYAHDYWGQGTSVTVSS
>Heavy Chain of SC16.4 IgG histidine scanning variant #36
(SEQ ID NO: 156)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGYAMHYWGQGTSVTVSS
>Heavy Chain of SC16.4 IgG histidine scanning variant #37
(SEQ ID NO: 157)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYDGYAMDHWGQGTSVTVSS
>Light Chain of SC16.4 IgG histidine scanning variant #1
(SEQ ID NO: 158)
DIQMTQTTSSLSASLGDRVTISCHASQDISNYLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #2
(SEQ ID NO: 159)
DIQMTQTTSSLSASLGDRVTISCRHSQDISNYLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #3
(SEQ ID NO: 160)
DIQMTQTTSSLSASLGDRVTISCRAHQDISNYLNWYQQKPDGTVKLLIYYTSRLHSGVPSRFSGS
GSGTDYSLTISNLELEDIATYFCQQGDMLPWTFGGGTKLEIK
>Light Chain of SC16.4 IgG histidine scanning variant #4
(SEQ ID NO: 161)
DIQMTQTTSSLSASLGDRVTISCRASHDISNYLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #5
(SEQ ID NO: 162)
DIQMTQTTSSLSASLGDRVTISCRASQHISNYLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #6
(SEQ ID NO: 163)
DIQMTQTTSSLSASLGDRVTISCRASQDHSNYLNWYQQKPDGTVKLLIYYTSRLHSGVPSRFSGS
GSGTDYSLTISNLELEDIATYFCQQGDMLPWTFGGGTKLEIK
>Light Chain of SC16.4 IgG histidine scanning variant #7
(SEQ ID NO: 164)
DIQMTQTTSSLSASLGDRVTISCRASQDIHNYLNWYQQKPDGTVKLLIYYTSRLHSGVPSRFSGS
GSGTDYSLTISNLELEDIATYFCQQGDMLPWTFGGGTKLEIK
>Light Chain of SC16.4 IgG histidine scanning variant #8
(SEQ ID NO: 165)
DIQMTQTTSSLSASLGDRVTISCRASQDISHYLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #9
(SEQ ID NO: 166)
DIQMTQTTSSLSASLGDRVTISCRASQDISNHLNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #10
(SEQ ID NO: 167)
DIQMTQTTSSLSASLGDRVTISCRASQDISNYHNWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #11
(SEQ ID NO: 168)
DIQMTQTTSSLSASLGDRVTISCRASQDISNYLHWYQQKPDGTVKLLIYYTSRLHSGVPSRFSGSG
SGTDYSLTISNLELEDIATYFCQQGDMLPWTFGGGTKLEIK
>Light Chain of SC16.4 IgG histidine scanning variant #12
(SEQ ID NO: 169)
DIQMTQTTSSLSASLGDRVTISCRASQDISNYLNWYQQKPDGTVKLLIYYHSRLHSGVPSRFSGSG
SGTDYSLTISNLELEDIATYFCQQGDMLPWTFGGGTKLEIK
>Light Chain of SC16.4 IgG histidine scanning variant #13
(SEQ ID NO: 170)
DIQMTQTTSSLSASLGDRVTISCRASQDISNYLNWYQQKPDGTVKLLIYYTHRLHSGVPSRFSGS
GSGTDYSLTISNLELEDIATYFCQQGDMLPWTFGGGTKLEIK
>Light Chain of SC16.4 IgG histidine scanning variant #14
(SEQ ID NO: 171)
DIQMTQTTSSLSASLGDRVTISCRASQDISNYLNWYQQKPDGTVKLLIYYTSHLHSGVPSRFSGSG
SGTDYSLTISNLELEDIATYFCQQGDMLPWTFGGGTKLEIK
>Light Chain of SC16.4 IgG histidine scanning variant #15
(SEQ ID NO: 172)
DIQMTQTTSSLSASLGDRVTISCRASQDISNYLNWYQQKPDGTVKLLIYYTSRHHSGVPSRFSGSG
SGTDYSLTISNLELEDIATYFCQQGDMLPWTFGGGTKLEIK
>Light Chain of SC16.4 IgG histidine scanning variant #16
(SEQ ID NO: 173)
DIQMTQTTSSLSASLGDRVTISCRASQDISNYLNWYQQKPDGTVKLLIYYTSRLASGVPSRFSGSG
SGTDYSLTISNLELEDIATYFCQQGDMLPWTFGGGTKLEIK
>Light Chain of SC16.4 IgG histidine scanning variant #17
(SEQ ID NO: 174)
DIQMTQTTSSLSASLGDRVTISCRASQDISNYLNWYQQKPDGTVKLLIYYTSRLHHGVPSRFSGS
GSGTDYSLTISNLELEDIATYFCQQGDMLPWTFGGGTKLEIK
>Light Chain of SC16.4 IgG histidine scanning variant #18
(SEQ ID NO: 175)
DIQMTQTTSSLSASLGDRVTISCRASQDISNYLNWYQQKPDGTVKLLIYYTSRLHSGVPSRFSGSG
SGTDYSLTISNLELEDIATYFCHQGDMLPWTFGGGTKLEIK
>Light Chain of SC16.4 IgG histidine scanning variant #19
(SEQ ID NO: 176)
DIQMTQTTSSLSASLGDRVTISCRASQDISNYLNWYQQKPDGTVKLLIYYTSRLHSGVPSRFSGSG
SGTDYSLTISNLELEDIATYFCQHGDMLPWTFGGGTKLEIK
>Light Chain of SC16.4 IgG histidine scanning variant #20
(SEQ ID NO: 177)
DIQMTQTTSSLSASLGDRVTISCRASQDISNYLNWYQQKPDGTVKLLIYYTSRLHSGVPSRFSGSG
SGTDYSLTISNLELEDIATYFCQQHDMLPWTFGGGTKLEIK
>Light Chain of SC16.4 IgG histidine scanning variant #21
(SEQ ID NO: 178)
DIQMTQTTSSLSASLGDRVTISCRASQDISNYLNWYQQKPDGTVKLLIYYTSRLHSGVPSRFSGSG
SGTDYSLTISNLELEDIATYFCQQGHMLPWTFGGGTKLEIK
>Light Chain of SC16.4 IgG histidine scanning variant #22
(SEQ ID NO: 179)
DIQMTQTTSSLSASLGDRVTISCRASQDISNYLNWYQQKPDGTVKLLIYYTSRLHSGVPSRFSGSG
SGTDYSLTISNLELEDIATYFCQQGDHLPWTFGGGTKLEIK
>Light Chain of SC16.4 IgG histidine scanning variant #23
(SEQ ID NO: 180)
DIQMTQTTSSLSASLGDRVTISCRASQDISNYLNWYQQKPDGTVKLLIYYTSRLHSGVPSRFSGSG
SGTDYSLTISNLELEDIATYFCQQGDMHPWTFGGGTKLEIK
>Light Chain of SC16.4 IgG histidine scanning variant #24
(SEQ ID NO: 181)
DIQMTQTTSSLSASLGDRVTISCRASQDISNYLNWYQQKPDGTVKLLIYYTSRLHSGVPSRFSGSG
SGTDYSLTISNLELEDIATYFCQQGDMLHWTFGGGTKLEIK
>Light Chain of SC16.4 IgG histidine scanning variant #25
(SEQ ID NO: 182)
DIQMTQTTSSLSASLGDRVTISCRASQDISNYLNWYQQKPDGTVKLLIYYTSRLHSGVPSRFSGSG
SGTDYSLTISNLELEDIATYFCQQGDMLPHTFGGGTKLEIK
>Light Chain of SC16.4 IgG histidine scanning variant #26
(SEQ ID NO: 183)
DIQMTQTTSSLSASLGDRVTISCRASQDISNYLNWYQQKPDGTVKLLIYYTSRLHSGVPSRFSGSG
SGTDYSLTISNLELEDIATYFCQQGDMLPWHFGGGTKLEIK
>Light Chain of SC16.4 IgG histidine scanning variant #27
(SEQ ID NO: 184)
DIQMTQTTSSLSASLGDRVTISCRASQDISNYLNWYQQKPDGTVKLLIYHTSRLHSGVPSRFSGSG
SGTDYSLTISNLELEDIATYFCQQGDMLPWTFGGGTKLEIK
>Heavy Chain Combinations of SC16.4 IgG histidine scanning
variant #1
(SEQ ID NO: 185)
QIQLVQSGPELKKPGETVKISCKASGYTFTDHSMHWVKQAPGKGLKWMGWINTETGHPGYAD
DFKGRFAFSLETSASTAYLQINNLKNEDTATYFCARYDGYAMDYWGQGTSVTVSS
>Heavy Chain Combinations of SC16.4 IgG histidine scanning
variant #2
(SEQ ID NO: 186)
QIQLVQSGPELKKPGETVKISCKASGYTFTDHSMHWVKQAPGKGLKWMGWINTETGEPGYAHD
FKGRFAFSLETSASTAYLQINNLKNEDTATYFCARYDGYAMDYWGQGTSVTVSS
>Heavy Chain Combinations of SC16.4 IgG histidine scanning
variant #3
(SEQ ID NO: 187)
QIQLVQSGPELKKPGETVKISCKASGYTFTDHSMHWVKQAPGKGLKWMGWINTETGEPGYADD
FKGRFAFSLETSASTAYLQINNLKNEDTATYFCARHDGYAMDYWGQGTSVTVSS
>Heavy Chain Combinations of SC16.4 IgG histidine scanning
variant #4
(SEQ ID NO: 188)
QIQLVQSGPELKKPGETVKISCKASGYTFTDHSMHWVKQAPGKGLKWMGWINTETGEPGYADD
FKGRFAFSLETSASTAYLQINNLKNEDTATYFCARYDGYAMHYWGQGTSVTVSS
>Heavy Chain Combinations of SC16.4 IgG histidine scanning
variant #5
(SEQ ID NO: 189)
QIQLVQSGPELKKPGETVKISCKASGYTFTDYSMHWVKQAPGKGLKWMGWINTETGHPGYAH
DFKGRFAFSLETSASTAYLQINNLKNEDTATYFCARYDGYAMDYWGQGTSVTVSS
>Heavy Chain Combinations of SC16.4 IgG histidine scanning
variant #6
(SEQ ID NO: 190)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARHDGYAMDYWGQGTSVTVSS
>Heavy Chain Combinations of SC16.4 IgG histidine scanning
variant #7
(SEQ ID NO: 191)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARYDGYAMHYWGQGTSVTVSS
>Heavy Chain Combinations of SC16.4 IgG histidine scanning
variant #8
(SEQ ID NO: 192)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARHDGYAMDYWGQGTSVTVSS
>Heavy Chain Combinations of SC16.4 IgG histidine scanning
variant #9
(SEQ ID NO: 193)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARYDGYAMHYWGQGTSVTVSS
>Heavy Chain Combinations of SC16.4 IgG histidine scanning
variant #10
(SEQ ID NO: 194)
QIQLVQSGPELKKPGETVKISCKASGYTFTDYSMHWVKQAPGKGLKWMGWINTETGEPGYADD
FKGRFAFSLETSASTAYLQINNLKNEDTATYFCARHDGYAMHYWGQGTSVTVSS
>Heavy Chain Combinations of SC16.4 IgG histidine scanning
variant #11
(SEQ ID NO: 195)
QIQLVQSGPELKKPGETVKISCKASGYTFTDHSMHWVKQAPGKGLKWMGWINTETGHPGYAH
DFKGRFAFSLETSASTAYLQINNLKNEDTATYFCARYDGYAMDYWGQGTSVTVSS
>Heavy Chain Combinations of SC16.4 IgG histidine scanning
variant #12
(SEQ ID NO: 196)
QIQLVQSGPELKKPGETVKISCKASGYTFTDHSMHWVKQAPGKGLKWMGWINTETGHPGYAD
DFKGRFAFSLETSASTAYLQINNLKNEDTATYFCARHDGYAMDYWGQGTSVTVSS
>Heavy Chain Combinations of SC16.4 IgG histidine scanning
variant #13
(SEQ ID NO: 197)
QIQLVQSGPELKKPGETVKISCKASGYTFTDHSMHWVKQAPGKGLKWMGWINTETGHPGYAD
DFKGRFAFSLETSASTAYLQINNLKNEDTATYFCARYDGYAMHYWGQGTSVTVSS
>Heavy Chain Combinations of SC16.4 IgG histidine scanning
variant #14
(SEQ ID NO: 198)
QIQLVQSGPELKKPGETVKISCKASGYTFTDHSMHWVKQAPGKGLKWMGWINTETGEPGYAHD
FKGRFAFSLETSASTAYLQINNLKNEDTATYFCARHDGYAMDYWGQGTSVTVSS
>Heavy Chain Combinations of SC16.4 IgG histidine scanning
variant #15
(SEQ ID NO: 199)
QIQLVQSGPELKKPGETVKISCKASGYTFTDHSMHWVKQAPGKGLKWMGWINTETGEPGYAHD
FKGRFAFSLETSASTAYLQINNLKNEDTATYFCARYDGYAMHYWGQGTSVTVSS
>Heavy Chain Combinations of SC16.4 IgG histidine scanning
variant #16
(SEQ ID NO: 200)
QIQLVQSGPELKKPGETVKISCKASGYTFTDHSMHWVKQAPGKGLKWMGWINTETGEPGYADD
FKGRFAFSLETSASTAYLQINNLKNEDTATYFCARHDGYAMHYWGQGTSVTVSS
>Heavy Chain Combinations of SC16.4 IgG histidine scanning
variant #17
(SEQ ID NO: 201)
QIQLVQSGPELKKPGETVKISCKASGYTFTDYSMHWVKQAPGKGLKWMGWINTETGHPGYAH
DFKGRFAFSLETSASTAYLQINNLKNEDTATYFCARHDGYAMDYWGQGTSVTVSS
>Heavy Chain Combinations of SC16.4 IgG histidine scanning
variant #18
(SEQ ID NO: 202)
QIQLVQSGPELKKPGETVKISCKASGYTFTDYSMHWVKQAPGKGLKWMGWINTETGHPGYAH
DFKGRFAFSLETSASTAYLQINNLKNEDTATYFCARYDGYAMHYWGQGTSVTVSS
>Heavy Chain Combinations of SC16.4 IgG histidine scanning
variant #19
(SEQ ID NO: 203)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARHDGYAMHYWGQGTSVTVSS
>Heavy Chain Combinations of SC16.4 IgG histidine scanning
variant #20
(SEQ ID NO: 204)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARHDGYAMHYWGQGTSVTVSS
>Heavy Chain Combinations of SC16.4 IgG histidine scanning
variant #21
(SEQ ID NO: 205)
QIQLVQSGPELKKPGETVKISCKASGYTFTDHSMHWVKQAPGKGLKWMGWINTETGHPGYAH
DFKGRFAFSLETSASTAYLQINNLKNEDTATYFCARHDGYAMDYWGQGTSVTVSS
>Heavy Chain Combinations of SC16.4 IgG histidine scanning
variant #22
(SEQ ID NO: 206)
QIQLVQSGPELKKPGETVKISCKASGYTFTDHSMHWVKQAPGKGLKWMGWINTETGHPGYAH
DFKGRFAFSLETSASTAYLQINNLKNEDTATYFCARYDGYAMHYWGQGTSVTVSS
>Heavy Chain Combinations of SC16.4 IgG histidine scanning
variant #23
(SEQ ID NO: 207)
QIQLVQSGPELKKPGETVKISCKASGYTFTDHSMHWVKQAPGKGLKWMGWINTETGHPGYAD
DFKGRFAFSLETSASTAYLQINNLKNEDTATYFCARHDGYAMHYWGQGTSVTVSS
>Heavy Chain Combinations of SC16.4 IgG histidine scanning
variant #24
(SEQ ID NO: 208)
QIQLVQSGPELKKPGETVKISCKASGYTFTDHSMHWVKQAPGKGLKWMGWINTETGEPGYAHD
FKGRFAFSLETSASTAYLQINNLKNEDTATYFCARHDGYAMHYWGQGTSVTVSS
>Heavy Chain Combinations of SC16.4 IgG histidine scanning
variant #25
(SEQ ID NO: 209)
QIQLVQSGPELKKPGETVKISCKASGYTFTDYSMHWVKQAPGKGLKWMGWINTETGHPGYAH
DFKGRFAFSLETSASTAYLQINNLKNEDTATYFCARHDGYAMHYWGQGTSVTVSS
>Heavy Chain Combinations of SC16.4 IgG histidine scanning
variant #26
(SEQ ID NO: 210)
QIQLVQSGPELKKPGETVKISCKASGYTFTDHSMHWVKQAPGKGLKWMGWINTETGHPGYAH
DFKGRFAFSLETSASTAYLQINNLKNEDTATYFCARHDGYAMHYWGQGTSVTVSS
>Heavy Chain Combinations of SC16.4 IgG histidine scanning
variant #27
(SEQ ID NO: 211)
QIQLVQSGPELKKPGETVKISCKASGYTFTDHSMHWVKQAPGKGLKWMGWINTETGEPGYADD
FKGRFAFSLETSASTAYLQINNLKNEDTATYFCARYHGYAMDYWGQGTSVTVSS
>Heavy Chain Combinations of SC16.4 IgG histidine scanning
variant #28
(SEQ ID NO: 212)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARYHGYAMDYWGQGTSVTVSS
>Heavy Chain Combinations of SC16.4 IgG histidine scanning
variant #29
(SEQ ID NO: 213)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARYHGYAMDYWGQGTSVTVSS
>Heavy Chain Combinations of SC16.4 IgG histidine scanning
variant #30
(SEQ ID NO: 214)
QIQLVQSGPELKKPGETVKISCKASGYTFTDYSMHWVKQAPGKGLKWMGWINTETGEPGYADD
FKGRFAFSLETSASTAYLQINNLKNEDTATYFCARHHGYAMDYWGQGTSVTVSS
>Heavy Chain Combinations of SC16.4 IgG histidine scanning
variant #31
(SEQ ID NO: 215)
QIQLVQSGPELKKPGETVKISCKASGYTFTDYSMHWVKQAPGKGLKWMGWINTETGEPGYADD
FKGRFAFSLETSASTAYLQINNLKNEDTATYFCARYHGYAMHYWGQGTSVTVSS
>Heavy Chain Combinations of SC16.4 IgG histidine scanning
variant #32
(SEQ ID NO: 216)
QIQLVQSGPELKKPGETVKISCKASGYTFTDHSMHWVKQAPGKGLKWMGWINTETGHPGYAD
DFKGRFAFSLETSASTAYLQINNLKNEDTATYFCARYHGYAMDYWGQGTSVTVSS
>Heavy Chain Combinations of SC16.4 IgG histidine scanning
variant #33
(SEQ ID NO: 217)
QIQLVQSGPELKKPGETVKISCKASGYTFTDHSMHWVKQAPGKGLKWMGWINTETGEPGYAHD
FKGRFAFSLETSASTAYLQINNLKNEDTATYFCARYHGYAMDYWGQGTSVTVSS
>Heavy Chain Combinations of SC16.4 IgG histidine scanning
variant #34
(SEQ ID NO: 218)
QIQLVQSGPELKKPGETVKISCKASGYTFTDHSMHWVKQAPGKGLKWMGWINTETGEPGYADD
FKGRFAFSLETSASTAYLQINNLKNEDTATYFCARHHGYAMDYWGQGTSVTVSS
>Heavy Chain Combinations of SC16.4 IgG histidine scanning
variant #35
(SEQ ID NO: 219)
QIQLVQSGPELKKPGETVKISCKASGYTFTDHSMHWVKQAPGKGLKWMGWINTETGEPGYADD
FKGRFAFSLETSASTAYLQINNLKNEDTATYFCARYHGYAMHYWGQGTSVTVSS
>Heavy Chain Combinations of SC16.4 IgG histidine scanning
variant #36
(SEQ ID NO: 220)
QIQLVQSGPELKKPGETVKISCKASGYTFTDYSMHWVKQAPGKGLKWMGWINTETGHPGYAH
DFKGRFAFSLETSASTAYLQINNLKNEDTATYFCARYHGYAMDYWGQGTSVTVSS
>Heavy Chain Combinations of SC16.4 IgG histidine scanning
variant #37
(SEQ ID NO: 221)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARHEIGYAMDYWGQGTSVTVSS
>Heavy Chain Combinations of SC16.4 IgG histidine scanning
variant #38
(SEQ ID NO: 222)
QIQLVQSGPELKKPGETVKISCKASGYTFTDYSMHWVKQAPGKGLKWMGWINTETGHPGYAD
DFKGRFAFSLETSASTAYLQINNLKNEDTATYFCARYHGYAMHYWGQGTSVTVSS
>Heavy Chain Combinations of SC16.4 IgG histidine scanning
variant #39
(SEQ ID NO: 223)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARHHGYAMDYWGQGTSVTVSS
>Heavy Chain Combinations of SC16.4 IgG histidine scanning
variant #40
(SEQ ID NO: 224)
QIQLVQSGPELKKPGETVKISCKASGYTFTDYSMHWVKQAPGKGLKWMGWINTETGEPGYAHD
FKGRFAFSLETSASTAYLQINNLKNEDTATYFCARYHGYAMHYWGQGTSVTVSS
>Heavy Chain Combinations of SC16.4 IgG histidine scanning
variant #41
(SEQ ID NO: 225)
QIQLVQSGPELKKPGETVKISCKASGYTFTDYSMHWVKQAPGKGLKWMGWINTETGEPGYADD
FKGRFAFSLETSASTAYLQINNLKNEDTATYFCARHHGYAMHYWGQGTSVTVSS
>Light Chain Combinations of SC16.4 IgG histidine scanning
variant #1
(SEQ ID NO: 226)
DIQMTQTTSSLSASLGDRVTISCRASQDHSNHLNWYQQKPDGTVKLLIYYTSRLHSGVPSRFSGS
GSGTDYSLTISNLELEDIATYFCQQGDMLPWTFGGGTKLEIK
>Light Chain Combinations of SC16.4 IgG histidine scanning
variant #2
(SEQ ID NO: 227)
DIQMTQTTSSLSASLGDRVTISCRASQDHSNYLHWYQQKPDGTVKLLIYYTSRLHSGVPSRFSGS
GSGTDYSLTISNLELEDIATYFCQQGDMLPWTFGGGTKLEIK
>Light Chain Combinations of SC16.4 IgG histidine scanning
variant #3
(SEQ ID NO: 228)
DIQMTQTTSSLSASLGDRVTISCRASQDHSNYLNWYQQKPDGTVKLLIYYTSRLHSGVPSRFSGS
GSGTDYSLTISNLELEDIATYFCQQGDHLPWTFGGGTKLEIK
>Light Chain Combinations of SC16.4 IgG histidine scanning
variant #4
(SEQ ID NO: 229)
DIQMTQTTSSLSASLGDRVTISCRASQDISNHLHWYQQKPDGTVKLLIYYTSRLHSGVPSRFSGSG
SGTDYSLTISNLELEDIATYFCQQGDMLPWTFGGGTKLEIK
>Light Chain Combinations of SC16.4 IgG histidine scanning
variant #5
(SEQ ID NO: 230)
DIQMTQTTSSLSASLGDRVTISCRASQDISNHLNWYQQKPDGTVKLLIYYTSRLHSGVPSRFSGSG
SGTDYSLTISNLELEDIATYFCQQGDHLPWTFGGGTKLEIK
>Light Chain Combinations of SC16.4 IgG histidine scanning
variant #6
(SEQ ID NO: 231)
DIQMTQTTSSLSASLGDRVTISCRASQDISNYLHWYQQKPDGTVKLLIYYTSRLHSGVPSRFSGSG
SGTDYSLTISNLELEDIATYFCQQGDHLPWTFGGGTKLEIK
>Light Chain Combinations of SC16.4 IgG histidine scanning
variant #7
(SEQ ID NO: 232)
DIQMTQTTSSLSASLGDRVTISCRASQDHSNHLHWYQQKPDGTVKLLIYYTSRLHSGVPSRFSGS
GSGTDYSLTISNLELEDIATYFCQQGDMLPWTFGGGTKLEIK
>Light Chain Combinations of SC16.4 IgG histidine scanning
variant #8
(SEQ ID NO: 233)
DIQMTQTTSSLSASLGDRVTISCRASQDHSNHLNWYQQKPDGTVKLLIYYTSRLHSGVPSRFSGS
GSGTDYSLTISNLELEDIATYFCQQGDHLPWTFGGGTKLEIK
>Light Chain Combinations of SC16.4 IgG histidine scanning
variant #9
(SEQ ID NO: 234)
DIQMTQTTSSLSASLGDRVTISCRASQDHSNYLHWYQQKPDGTVKLLIYYTSRLHSGVPSRFSGS
GSGTDYSLTISNLELEDIATYFCQQGDHLPWTFGGGTKLEIK
>Light Chain Combinations of SC16.4 IgG histidine scanning
variant #10
(SEQ ID NO: 235)
DIQMTQTTSSLSASLGDRVTISCRASQDISNHLHWYQQKPDGTVKLLIYYTSRLHSGVPSRFSGSG
SGTDYSLTISNLELEDIATYFCQQGDHLPWTFGGGTKLEIK
>Heavy Chain of hSC16.13 IgG
(SEQ ID NO: 236)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Light Chain of hSC16.13 IgG
(SEQ ID NO: 237)
DIQMTQSPSSLSASVGDRVTITCSASSSVSYMYWYQQKPGKAPKWYLTSNLASGVPSRFSGSGS
GTDFTLTISSLQPEDFATYYCQQWRSNPFTFGQGTKLEIK
>Heavy Chain of hSC16.13 IgG histidine scanning variant #1
(SEQ ID NO: 238)
QITLKESGPTLVKPTQTLTLTCTFSHFSLSTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #2
(SEQ ID NO: 239)
QITLKESGPTLVKPTQTLTLTCTFSGHSLSTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #3
(SEQ ID NO: 240)
QITLKESGPTLVKPTQTLTLTCTFSGFHLSTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #4
(SEQ ID NO: 241)
QITLKESGPTLVKPTQTLTLTCTFSGFSHSTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #5
(SEQ ID NO: 242)
QITLKESGPTLVKPTQTLTLTCTFSGFSLHTSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #6
(SEQ ID NO: 243)
QITLKESGPTLVKPTQTLTLTCTFSGFSLSHSGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #7
(SEQ ID NO: 244)
QITLKESGPTLVKPTQTLTLTCTFSGFSLSTHGMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #8
(SEQ ID NO: 245)
QITLKESGPTLVKPTQTLTLTCTFSGFSLSTSHMGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #9
(SEQ ID NO: 246)
QITLKESGPTLVKPTQTLTLTCTFSGFSLSTSGHGVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #10
(SEQ ID NO: 247)
QITLKESGPTLVKPTQTLTLTCTFSGFSLSTSGMHVGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #11
(SEQ ID NO: 248)
QITLKESGPTLVKPTQTLTLTCTFSGFSLSTSGMGHGWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #12
(SEQ ID NO: 249)
QITLKESGPTLVKPTQTLTLTCTFSGFSLSTSGMGVHWIRQPPGKALEWLAHIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #13
(SEQ ID NO: 250)
QITLKESGPTLVKPTQTLTLTCTFSGFSLSTSGMGVGWIRQPPGKALEWLAAIWWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #14
(SEQ ID NO: 251)
QITLKESGPTLVKPTQTLTLTCTFSGFSLSTSGMGVGWIRQPPGKALEWLAHHWWDDVKRYSPS
LKSRLTITKDTSKNQVVLTMTNMDPVDTATYYCARIVSFDNDVVSAMDYWGQGTLVTVSS
>Heavy Chain of hSC16.13 IgG histidine scanning variant #15
(SEQ ID NO: 252)
QITLKESGPTLVKPTQTLTLTCTFSGFSLSTSGMGVGWIRQPPGKALEWLAHIHWDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #16
(SEQ ID NO: 253)
QITLKESGPTLVKPTQTLTLTCTFSGFSLSTSGMGVGWIRQPPGKALEWLAHIWHDDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #17
(SEQ ID NO: 254)
QITLKESGPTLVKPTQTLTLTCTFSGFSLSTSGMGVGWIRQPPGKALEWLAHIWWHDVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #18
(SEQ ID NO: 255)
QITLKESGPTLVKPTQTLTLTCTFSGFSLSTSGMGVGWIRQPPGKALEWLAHIWWDHVKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #19
(SEQ ID NO: 256)
QITLKESGPTLVKPTQTLTLTCTFSGFSLSTSGMGVGWIRQPPGKALEWLAHIWWDDHKRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #20
(SEQ ID NO: 257)
QITLKESGPTLVKPTQTLTLTCTFSGFSLSTSGMGVGWIRQPPGKALEWLAHIWWDDVHRYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #21
(SEQ ID NO: 258)
QITLKESGPTLVKPTQTLTLTCTFSGFSLSTSGMGVGWIRQPPGKALEWLAHIWWDDVKHYSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #22
(SEQ ID NO: 259)
QITLKESGPTLVKPTQTLTLTCTFSGFSLSTSGMGVGWIRQPPGKALEWLAHIWWDDVKRHSPSL
KSRLTITKDTSKNQVVLTMTNMDPVDTATYYCARIVSFDNDVVSAMDYWGQGTLVTVSS
>Heavy Chain of hSC16.13 IgG histidine scanning variant #23
(SEQ ID NO: 260)
QITLKESGPTLVKPTQTLTLTCTFSGFSLSTSGMGVGWIRQPPGKALEWLAHIWWDDVKRYHPSL
KSRLTITKDTSKNQVVLTMTNMDPVDTATYYCARIVSFDNDVVSAMDYWGQGTLVTVSS
>Heavy Chain of hSC16.13 IgG histidine scanning variant #24
(SEQ ID NO: 261)
QITLKESGPTLVKPTQTLTLTCTFSGFSLSTSGMGVGWIRQPPGKALEWLAHIWWDDVKRYSHSL
KSRLTITKDTSKNQVVLTMTNMDPVDTATYYCARIVSFDNDVVSAMDYWGQGTLVTVSS
>Heavy Chain of hSC16.13 IgG histidine scanning variant #25
(SEQ ID NO: 262)
QITLKESGPTLVKPTQTLTLTCTFSGFSLSTSGMGVGWIRQPPGKALEWLAHIWWDDVKRYSPHL
KSRLTITKDTSKNQVVLTMTNMDPVDTATYYCARIVSFDNDVVSAMDYWGQGTLVTVSS
>Heavy Chain of hSC16.13 IgG histidine scanning variant #26
(SEQ ID NO: 263)
QITLKESGPTLVKPTQTLTLTCTFSGFSLSTSGMGVGWIRQPPGKALEWLAHIWWDDVKRYSPSH
KSRLTITKDTSKNQVVLTMTNMDPVDTATYYCARIVSFDNDVVSAMDYWGQGTLVTVSS
>Heavy Chain of hSC16.13 IgG histidine scanning variant #27
(SEQ ID NO: 264)
QITLKESGPTLVKPTQTLTLTCTFSGFSLSTSGMGVGWIRQPPGKALEWLAHIWWDDVKRYSPSL
HSRLTITKDTSKNQVVLTMTNMDPVDTATYYCARIVSFDNDVVSAMDYWGQGTLVTVSS
>Heavy Chain of hSC16.13 IgG histidine scanning variant #28
(SEQ ID NO: 265)
QITLKESGPTLVKPTQTLTLTCTFSGFSLSTSGMGVGWIRQPPGKALEWLAHIWWDDVKRYSPSL
KHRLTITKDTSKNQVVLTMTNMDPVDTATYYCARIVSFDNDVVSAMDYWGQGTLVTVSS
>Heavy Chain of hSC16.13 IgG histidine scanning variant #29
(SEQ ID NO: 266)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCHRIVSFDNDVVSAMDYWGQGTLVTVSS
>Heavy Chain of hSC16.13 IgG histidine scanning variant #30
(SEQ ID NO: 267)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCAHIVSFDNDVVSAMDYWGQGTLVTVSS
>Heavy Chain of hSC16.13 IgG histidine scanning variant #31
(SEQ ID NO: 268)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARHVSFDNDVVSAMDYWGQGTLVTVSS
>Heavy Chain of hSC16.13 IgG histidine scanning variant #32
(SEQ ID NO: 269)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIHSFDNDVVSAMDYWGQGTLVTVSS
>Heavy Chain of hSC16.13 IgG histidine scanning variant #33
(SEQ ID NO: 270)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVHFDNDVVSAMDYWGQGTLVTVSS
>Heavy Chain of hSC16.13 IgG histidine scanning variant #34
(SEQ ID NO: 271)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSHDNDVVSAMDYWGQGTLVTVSS
>Heavy Chain of hSC16.13 IgG histidine scanning variant #35
(SEQ ID NO: 272)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFHNDVVSAMDYWGQGTLVTVSS
>Heavy Chain of hSC16.13 IgG histidine scanning variant #36
(SEQ ID NO: 273)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDHDVVSAMDYWGQGTLVTVSS
>Heavy Chain of hSC16.13 IgG histidine scanning variant #37
(SEQ ID NO: 274)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNHVVSAMDYWGQGTLVTVSS
>Heavy Chain of hSC16.13 IgG histidine scanning variant #38
(SEQ ID NO: 275)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDHVSAMDYWGQGTLVTVSS
>Heavy Chain of hSC16.13 IgG histidine scanning variant #39
(SEQ ID NO: 276)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVHSAMDYWGQGTLVTVSS
>Heavy Chain of hSC16.13 IgG histidine scanning variant #40
(SEQ ID NO: 277)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVHAMDYWGQGTLVTVSS
>Heavy Chain of hSC16.13 IgG histidine scanning variant #41
(SEQ ID NO: 278)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVSHMDYWGQGTLVTVSS
>Heavy Chain of hSC16.13 IgG histidine scanning variant #42
(SEQ ID NO: 279)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVSAHDYWGQGTLVTVSS
>Heavy Chain of hSC16.13 IgG histidine scanning variant #43
(SEQ ID NO: 280)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVSAMHYWGQGTLVTVSS
>Heavy Chain of hSC16.13 IgG histidine scanning variant #44
(SEQ ID NO: 281)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVSFDNDVVSAMDHWGQGTLVTVSS
>Light Chain of hSC16.13 IgG histidine scanning variant #1
(SEQ ID NO: 282)
DIQMTQSPSSLSASVGDRVTITCHASSSVSYMYWYQQKPGKAPKWYLTSNLASGVPSRFSGSG
SGTDFTLTISSLQPEDFATYYCQQWRSNPFTFGQGTKLEIK
>Light Chain of hSC16.13 IgG histidine scanning variant #2
(SEQ ID NO: 283)
DIQMTQSPSSLSASVGDRVTITCSHSSSVSYMYWYQQKPGKAPKLLIYLTSNLASGVPSRFSGSGS
GTDFTLTISSLQPEDFATYYCQQWRSNPFTFGQGTKLEIK
>Light Chain of hSC16.13 IgG histidine scanning variant #3
(SEQ ID NO: 284)
DIQMTQSPSSLSASVGDRVTITCSAHSSVSYMYWYQQKPGKAPKLLIYLTSNLASGVPSRFSGSG
SGTDFTLTISSLQPEDFATYYCQQWRSNPFTFGQGTKLEIK
>Light Chain of hSC16.13 IgG histidine scanning variant #4
(SEQ ID NO: 285)
DIQMTQSPSSLSASVGDRVTITCSASHSVSYMYWYQQKPGKAPKLLIYLTSNLASGVPSRFSGSG
SGTDFTLTISSLQPEDFATYYCQQWRSNPFTFGQGTKLEIK
>Light Chain of hSC16.13 IgG histidine scanning variant #5
(SEQ ID NO: 286)
DIQMTQSPSSLSASVGDRVTITCSASSHVSYMYWYQQKPGKAPKLLIYLTSNLASGVPSRFSGSG
SGTDFTLTISSLQPEDFATYYCQQWRSNPFTFGQGTKLEIK
>Light Chain of hSC16.13 IgG histidine scanning variant #6
(SEQ ID NO: 287)
DIQMTQSPSSLSASVGDRVTITCSASSSHSYMYWYQQKPGKAPKLLIYLTSNLASGVPSRFSGSGS
GTDFTLTISSLQPEDFATYYCQQWRSNPFTFGQGTKLEIK
>Light Chain of hSC16.13 IgG histidine scanning variant #7
(SEQ ID NO: 288)
DIQMTQSPSSLSASVGDRVTITCSASSSVHYMYWYQQKPGKAPKLLIYLTSNLASGVPSRFSGSG
SGTDFTLTISSLQPEDFATYYCQQWRSNPFTFGQGTKLEIK
>Light Chain of hSC16.13 IgG histidine scanning variant #8
(SEQ ID NO: 289)
DIQMTQSPSSLSASVGDRVTITCSASSSVSHMYWYQQKPGKAPKLLIYLTSNLASGVPSRFSGSGS
GTDFTLTISSLQPEDFATYYCQQWRSNPFTFGQGTKLEIK
>Light Chain of hSC16.13 IgG histidine scanning variant #9
(SEQ ID NO: 290)
DIQMTQSPSSLSASVGDRVTITCSASSSVSYHYWYQQKPGKAPKLLIYLTSNLASGVPSRFSGSGS
GTDFTLTISSLQPEDFATYYCQQWRSNPFTFGQGTKLEIK
>Light Chain of hSC16.13 IgG histidine scanning variant #10
(SEQ ID NO: 291)
DIQMTQSPSSLSASVGDRVTITCSASSSVSYMHWYQQKPGKAPKLLIYLTSNLASGVPSRFSGSGS
GTDFTLTISSLQPEDFATYYCQQWRSNPFTFGQGTKLEIK
>Light Chain of hSC16.13 IgG histidine scanning variant #11
(SEQ ID NO: 292)
DIQMTQSPSSLSASVGDRVTITCSASSSVSYMYWYQQKPGKAPKLLIYHTSNLASGVPSRFSGSGS
GTDFTLTISSLQPEDFATYYCQQWRSNPFTFGQGTKLEIK
>Light Chain of hSC16.13 IgG histidine scanning variant #12
(SEQ ID NO: 293)
DIQMTQSPSSLSASVGDRVTITCSASSSVSYMYWYQQKPGKAPKWYLHSNLASGVPSRFSGSGS
GTDFTLTISSLQPEDFATYYCQQWRSNPFTFGQGTKLEIK
>Light Chain of hSC16.13 IgG histidine scanning variant #13
(SEQ ID NO: 294)
DIQMTQSPSSLSASVGDRVTITCSASSSVSYMYWYQQKPGKAPKWYLTHNLASGVPSRFSGSG
SGTDFTLTISSLQPEDFATYYCQQWRSNPFTFGQGTKLEIK
>Light Chain of hSC16.13 IgG histidine scanning variant #14
(SEQ ID NO: 295)
DIQMTQSPSSLSASVGDRVTITCSASSSVSYMYWYQQKPGKAPKWYLTSHLASGVPSRFSGSGS
GTDFTLTISSLQPEDFATYYCQQWRSNPFTFGQGTKLEIK
>Light Chain of hSC16.13 IgG histidine scanning variant #15
(SEQ ID NO: 296)
DIQMTQSPSSLSASVGDRVTITCSASSSVSYMYWYQQKPGKAPKWYLTSNHASGVPSRFSGSGS
GTDFTLTISSLQPEDFATYYCQQWRSNPFTFGQGTKLEIK
>Light Chain of hSC16.13 IgG histidine scanning variant #16
(SEQ ID NO: 297)
DIQMTQSPSSLSASVGDRVTITCSASSSVSYMYWYQQKPGKAPKWYLTSNLHSGVPSRFSGSGS
GTDFTLTISSLQPEDFATYYCQQWRSNPFTFGQGTKLEIK
>Light Chain of hSC16.13 IgG histidine scanning variant #17
(SEQ ID NO: 298)
DIQMTQSPSSLSASVGDRVTITCSASSSVSYMYWYQQKPGKAPKLLIYLTSNLAHGVPSRFSGSG
SGTDFTLTISSLQPEDFATYYCQQWRSNPFTFGQGTKLEIK
>Light Chain of hSC16.13 IgG histidine scanning variant #18
(SEQ ID NO: 299)
DIQMTQSPSSLSASVGDRVTITCSASSSVSYMYWYQQKPGKAPKWYLTSNLASGVPSRFSGSGS
GTDFTLTISSLQPEDFATYYCHQWRSNPFTFGQGTKLEIK
>Light Chain of hSC16.13 IgG histidine scanning variant #19
(SEQ ID NO: 300)
DIQMTQSPSSLSASVGDRVTITCSASSSVSYMYWYQQKPGKAPKWYLTSNLASGVPSRFSGSGS
GTDFTLTISSLQPEDFATYYCQHWRSNPFTFGQGTKLEIK
>Light Chain of hSC16.13 IgG histidine scanning variant #20
(SEQ ID NO: 301)
DIQMTQSPSSLSASVGDRVTITCSASSSVSYMYWYQQKPGKAPKLLIYLTSNLASGVPSRFSGSGS
GTDFTLTISSLQPEDFATYYCQQHRSNPFTFGQGTKLEIK
>Light Chain of hSC16.13 IgG histidine scanning variant #21
(SEQ ID NO: 302)
DIQMTQSPSSLSASVGDRVTITCSASSSVSYMYWYQQKPGKAPKLLIYLTSNLASGVPSRFSGSGS
GTDFTLTISSLQPEDFATYYCQQWHSNPFTFGQGTKLEIK
>Light Chain of hSC16.13 IgG histidine scanning variant #22
(SEQ ID NO: 303)
DIQMTQSPSSLSASVGDRVTITCSASSSVSYMYWYQQKPGKAPKLLIYLTSNLASGVPSRFSGSGS
GTDFTLTISSLQPEDFATYYCQQWRHNPFTFGQGTKLEIK
>Light Chain of hSC16.13 IgG histidine scanning variant #23
(SEQ ID NO: 304)
DIQMTQSPSSLSASVGDRVTITCSASSSVSYMYWYQQKPGKAPKLLIYLTSNLASGVPSRFSGSGS
GTDFTLTISSLQPEDFATYYCQQWRSHPFTFGQGTKLEIK
>Light Chain of hSC16.13 IgG histidine scanning variant #24
(SEQ ID NO: 305)
DIQMTQSPSSLSASVGDRVTITCSASSSVSYMYWYQQKPGKAPKLLIYLTSNLASGVPSRFSGSGS
GTDFTLTISSLQPEDFATYYCQQWRSNHFTFGQGTKLEIK
>Light Chain of hSC16.13 IgG histidine scanning variant #25
(SEQ ID NO: 306)
DIQMTQSPSSLSASVGDRVTITCSASSSVSYMYWYQQKPGKAPKLLIYLTSNLASGVPSRFSGSGS
GTDFTLTISSLQPEDFATYYCQQWRSNPHTFGQGTKLEIK
>Light Chain of hSC16.13 IgG histidine scanning variant #26
(SEQ ID NO: 307)
DIQMTQSPSSLSASVGDRVTITCSASSSVSYMYWYQQKPGKAPKLLIYLTSNLASGVPSRFSGSGS
GTDFTLTISSLQPEDFATYYCQQWRSNPFHFGQGTKLEIK
>Heavy Chain Combinations of hSC16.13 IgG histidine scanning
variant #1
(SEQ ID NO: 308)
QITLKESGPTLVKPTQTLTLTCTFSGHSLSTSGMGVGWIRQPPGKALEWLAAIWWDDVKRYSPSL
KSRLTITKDTSKNQVVLTMTNMDPVDTATYYCARIVSFDNDVVSAMDYWGQGTLVTVSS
>Heavy Chain Combinations of hSC16.13 IgG histidine scanning
variant #2
(SEQ ID NO: 309)
QITLKESGPTLVKPTQTLTLTCTFSGHSLSTSGMGVGWIRQPPGKALEWLAHIWHDDVKRYSPSL
KSRLTITKDTSKNQVVLTMTNMDPVDTATYYCARIVSFDNDVVSAMDYWGQGTLVTVSS
>Heavy Chain Combinations of hSC16.13 IgG histidine scanning
variant #3
(SEQ ID NO: 310)
QITLKESGPTLVKPTQTLTLTCTFSGHSLSTSGMGVGWIRQPPGKALEWLAHIWWDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #4
(SEQ ID NO: 311)
QITLKESGPTLVKPTQTLTLTCTFSGHSLSTSGMGVGWIRQPPGKALEWLAHIWWDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #5
(SEQ ID NO: 312)
QITLKESGPTLVKPTQTLTLTCTFSGFSLSTSGMGVGWIRQPPGKALEWLAAIWHDDVKRYSPSL
KSRLTITKDTSKNQVVLTMTNMDPVDTATYYCARIVSFDNDVVSAMDYWGQGTLVTVSS
>Heavy Chain Combinations of hSC16.13 IgG histidine scanning
variant #6
(SEQ ID NO: 313)
QITLKESGPTLVKPTQTLTLTCTFSGFSLSTSGMGVGWIRQPPGKALEWLAAIWWDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #7
(SEQ ID NO: 314)
QITLKESGPTLVKPTQTLTLTCTFSGFSLSTSGMGVGWIRQPPGKALEWLAAIWWDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #8
(SEQ ID NO: 315)
QITLKESGPTLVKPTQTLTLTCTFSGFSLSTSGMGVGWIRQPPGKALEWLAHIWHDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #9
(SEQ ID NO: 316)
QITLKESGPTLVKPTQTLTLTCTFSGFSLSTSGMGVGWIRQPPGKALEWLAHIWHDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #10
(SEQ ID NO: 317)
QITLKESGPTLVKPTQTLTLTCTFSGFSLSTSGMGVGWIRQPPGKALEWLAHIWWDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #11
(SEQ ID NO: 318)
QITLKESGPTLVKPTQTLTLTCTFSGHSLSTSGMGVGWIRQPPGKALEWLAAIWHDDVKRYSPSL
KSRLTITKDTSKNQVVLTMTNMDPVDTATYYCARIVSFDNDVVSAMDYWGQGTLVTVSS
>Heavy Chain Combinations of hSC16.13 IgG histidine scanning
variant #12
(SEQ ID NO: 319)
QITLKESGPTLVKPTQTLTLTCTFSGHSLSTSGMGVGWIRQPPGKALEWLAAIWWDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #13
(SEQ ID NO: 320)
QITLKESGPTLVKPTQTLTLTCTFSGHSLSTSGMGVGWIRQPPGKALEWLAAIWWDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #14
(SEQ ID NO: 321)
QITLKESGPTLVKPTQTLTLTCTFSGHSLSTSGMGVGWIRQPPGKALEWLAHIWHDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #15
(SEQ ID NO: 322)
QITLKESGPTLVKPTQTLTLTCTFSGHSLSTSGMGVGWIRQPPGKALEWLAHIWHDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #16
(SEQ ID NO: 323)
QITLKESGPTLVKPTQTLTLTCTFSGHSLSTSGMGVGWIRQPPGKALEWLAHIWWDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #17
(SEQ ID NO: 324)
QITLKESGPTLVKPTQTLTLTCTFSGFSLSTSGMGVGWIRQPPGKALEWLAAIWHDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #18
(SEQ ID NO: 325)
QITLKESGPTLVKPTQTLTLTCTFSGFSLSTSGMGVGWIRQPPGKALEWLAAIWHDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #19
(SEQ ID NO: 326)
QITLKESGPTLVKPTQTLTLTCTFSGFSLSTSGMGVGWIRQPPGKALEWLAAIWWDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #20
(SEQ ID NO: 327)
QITLKESGPTLVKPTQTLTLTCTFSGFSLSTSGMGVGWIRQPPGKALEWLAHIWHDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #21
(SEQ ID NO: 328)
QITLKESGPTLVKPTQTLTLTCTFSGHSLSTSGMGVGWIRQPPGKALEWLAAIWHDDVKRYSPSL
KSRLTITKDTSKNQVVLTMTNMDPVDTATYYCARIVHFDNDVVSAMDYWGQGTLVTVSS
>Heavy Chain Combinations of hSC16.13 IgG histidine scanning
variant #22
(SEQ ID NO: 329)
QITLKESGPTLVKPTQTLTLTCTFSGHSLSTSGMGVGWIRQPPGKALEWLAAIWHDDVKRYSPSL
KSRLTITKDTSKNQVVLTMTNMDPVDTATYYCARIVSFDNHVVSAMDYWGQGTLVTVSS
>Heavy Chain Combinations of hSC16.13 IgG histidine scanning
variant #23
(SEQ ID NO: 330)
QITLKESGPTLVKPTQTLTLTCTFSGHSLSTSGMGVGWIRQPPGKALEWLAAIWWDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #24
(SEQ ID NO: 331)
QITLKESGPTLVKPTQTLTLTCTFSGHSLSTSGMGVGWIRQPPGKALEWLAHIWHDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #25
(SEQ ID NO: 332)
QITLKESGPTLVKPTQTLTLTCTFSGFSLSTSGMGVGWIRQPPGKALEWLAAIWHDDVKRYSPSL
KSRLTITKDTSKNQVVLTMTNMDPVDTATYYCARIVHFDNHVVSAMDYWGQGTLVTVSS
>Heavy Chain Combinations of hSC16.13 IgG histidine scanning
variant #26
(SEQ ID NO: 333)
QITLKESGPTLVKPTQTLTLTCTFSGHSLSTSGMGVGWIRQPPGKALEWLAAIWHDDVKRYSPSL
KSRLTITKDTSKNQVVLTMTNMDPVDTATYYCARIVHFDNHVVSAMDYWGQGTLVTVSS
>Light Chain Combinations of SC16.4 IgG histidine scanning
variant #1
(SEQ ID NO: 334)
DIQMTQSPSSLSASVGDRVTITCSASSSVSYMHWYQQKPGKAPKLLIYHTSNLASGVPSRFSGSGS
GTDFTLTISSLQPEDFATYYCQQWRSNPFTFGQGTKLEIK
>Heavy Chain of hSC16.15 IgG
(SEQ ID NO: 335)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Light Chain of hSC16.15 IgG
(SEQ ID NO: 336)
AIQLTQSPSSLSASVGDRVTITCRASENIYYNLAWYQQKPGKAPKWYTANSLEDGVPSRFSGSG
SGTDFTLTISSLQPEDFATYFCKQAYDVPPTFGGGTKLEIK
>Heavy Chain of hSC16.15 IgG histidine scanning variant #1
(SEQ ID NO: 337)
QVQLVQSGAEVKKPGASVKVSCHASGYTFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #2
(SEQ ID NO: 338)
QVQLVQSGAEVKKPGASVKVSCKHSGYTFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #3
(SEQ ID NO: 339)
QVQLVQSGAEVKKPGASVKVSCKAHGYTFTRYWIHWIRQAPGQGLEWMGYINPTTVYTEFNQ
NFKDRVTMTRDTSTSTVYMELSSLRSEDTAVYYCARGGSNFFDYWGQGTTVTVSS
>Heavy Chain of hSC16.15 IgG histidine scanning variant #4
(SEQ ID NO: 340)
QVQLVQSGAEVKKPGASVKVSCKASHYTFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #5
(SEQ ID NO: 341)
QVQLVQSGAEVKKPGASVKVSCKASGHTFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #6
(SEQ ID NO: 342)
QVQLVQSGAEVKKPGASVKVSCKASGYHFTRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #7
(SEQ ID NO: 343)
QVQLVQSGAEVKKPGASVKVSCKASGYTHTRYWIHWIRQAPGQGLEWMGYINPTTVYTEFNQ
NFKDRVTMTRDTSTSTVYMELSSLRSEDTAVYYCARGGSNFFDYWGQGTTVTVSS
>Heavy Chain of hSC16.15 IgG histidine scanning variant #8
(SEQ ID NO: 344)
QVQLVQSGAEVKKPGASVKVSCKASGYTFHRYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #9
(SEQ ID NO: 345)
QVQLVQSGAEVKKPGASVKVSCKASGYTFTHYWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #10
(SEQ ID NO: 346)
QVQLVQSGAEVKKPGASVKVSCKASGYTFTRHWIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #11
(SEQ ID NO: 347)
QVQLVQSGAEVKKPGASVKVSCKASGYTFTRYHIHWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #12
(SEQ ID NO: 348)
QVQLVQSGAEVKKPGASVKVSCKASGYTFTRYWHHWIRQAPGQGLEWMGYINPTTVYTEFNQ
NFKDRVTMTRDTSTSTVYMELSSLRSEDTAVYYCARGGSNFFDYWGQGTTVTVSS
>Heavy Chain of hSC16.15 IgG histidine scanning variant #13
(SEQ ID NO: 349)
QVQLVQSGAEVKKPGASVKVSCKASGYTFTRYWIAWIRQAPGQGLEWMGYINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #14
(SEQ ID NO: 350)
QVQLVQSGAEVKKPGASVKVSCKASGYTFTRYWIHWIRQAPGQGLEWMGHINPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #15
(SEQ ID NO: 351)
QVQLVQSGAEVKKPGASVKVSCKASGYTFTRYWIHWIRQAPGQGLEWMGYHNPTTVYTEFNQ
NFKDRVTMTRDTSTSTVYMELSSLRSEDTAVYYCARGGSNFFDYWGQGTTVTVSS
>Heavy Chain of hSC16.15 IgG histidine scanning variant #16
(SEQ ID NO: 352)
QVQLVQSGAEVKKPGASVKVSCKASGYTFTRYWIHWIRQAPGQGLEWMGYIHPTTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #17
(SEQ ID NO: 353)
QVQLVQSGAEVKKPGASVKVSCKASGYTFTRYWIHWIRQAPGQGLEWMGYINHTTVYTEFNQ
NFKDRVTMTRDTSTSTVYMELSSLRSEDTAVYYCARGGSNFFDYWGQGTTVTVSS
>Heavy Chain of hSC16.15 IgG histidine scanning variant #18
(SEQ ID NO: 354)
QVQLVQSGAEVKKPGASVKVSCKASGYTFTRYWIHWIRQAPGQGLEWMGYINPHTVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #19
(SEQ ID NO: 355)
QVQLVQSGAEVKKPGASVKVSCKASGYTFTRYWIHWIRQAPGQGLEWMGYINPTHVYTEFNQN
FKDRVTMTRDTSTSTVYMELSSLRSEDTAVYYCARGGSNFFDYWGQGTTVTVSS
>Heavy Chain of hSC16.15 IgG histidine scanning variant #20
(SEQ ID NO: 356)
QVQLVQSGAEVKKPGASVKVSCKASGYTFTRYWIHWIRQAPGQGLEWMGYINPTTHYTEFNQN
FKDRVTMTRDTSTSTVYMELSSLRSEDTAVYYCARGGSNFFDYWGQGTTVTVSS
>Heavy Chain of hSC16.15 IgG histidine scanning variant #21
(SEQ ID NO: 357)
QVQLVQSGAEVKKPGASVKVSCKASGYTFTRYWIHWIRQAPGQGLEWMGYINPTTVHTEFNQN
FKDRVTMTRDTSTSTVYMELSSLRSEDTAVYYCARGGSNFFDYWGQGTTVTVSS
>Heavy Chain of hSC16.15 IgG histidine scanning variant #22
(SEQ ID NO: 358)
QVQLVQSGAEVKKPGASVKVSCKASGYTFTRYWIHWIRQAPGQGLEWMGYINPTTVYHEFNQN
FKDRVTMTRDTSTSTVYMELSSLRSEDTAVYYCARGGSNFFDYWGQGTTVTVSS
>Heavy Chain of hSC16.15 IgG histidine scanning variant #23
(SEQ ID NO: 359)
QVQLVQSGAEVKKPGASVKVSCKASGYTFTRYWIHWIRQAPGQGLEWMGYINPTTVYTHFNQN
FKDRVTMTRDTSTSTVYMELSSLRSEDTAVYYCARGGSNFFDYWGQGTTVTVSS
>Heavy Chain of hSC16.15 IgG histidine scanning variant #24
(SEQ ID NO: 360)
QVQLVQSGAEVKKPGASVKVSCKASGYTFTRYWIHWIRQAPGQGLEWMGYINPTTVYTEHNQ
NFKDRVTMTRDTSTSTVYMELSSLRSEDTAVYYCARGGSNFFDYWGQGTTVTVSS
>Heavy Chain of hSC16.15 IgG histidine scanning variant #25
(SEQ ID NO: 361)
QVQLVQSGAEVKKPGASVKVSCKASGYTFTRYWIHWIRQAPGQGLEWMGYINPTTVYTEFHQN
FKDRVTMTRDTSTSTVYMELSS LRS EDTAVYYCARGGSNFFDYWGQGTTVTVSS
>Heavy Chain of hSC16.15 IgG histidine scanning variant #26
(SEQ ID NO: 362)
QVQLVQSGAEVKKPGASVKVSCKASGYTFTRYWIHWIRQAPGQGLEWMGYINPTTVYTEFNHN
FKDRVTMTRDTSTSTVYMELSSLRSEDTAVYYCARGGSNFFDYWGQGTTVTVSS
>Heavy Chain of hSC16.15 IgG histidine scanning variant #27
(SEQ ID NO: 363)
QVQLVQSGAEVKKPGASVKVSCKASGYTFTRYWIHWIRQAPGQGLEWMGYINPTTVYTEFNQH
FKDRVTMTRDTSTSTVYMELSSLRSEDTAVYYCARGGSNFFDYWGQGTTVTVSS
>Heavy Chain of hSC16.15 IgG histidine scanning variant #28
(SEQ ID NO: 364)
QVQLVQSGAEVKKPGASVKVSCKASGYTFTRYWIHWIRQAPGQGLEWMGYINPTTVYTEFNQN
HKDRVTMTRDTSTSTVYMELSSLRSEDTAVYYCARGGSNFFDYWGQGTTVTVSS
>Heavy Chain of hSC16.15 IgG histidine scanning variant #29
(SEQ ID NO: 365)
QVQLVQSGAEVKKPGASVKVSCKASGYTFTRYWIHWIRQAPGQGLEWMGYINPTTVYTEFNQN
FHDRVTMTRDTSTSTVYMELSSLRSEDTAVYYCARGGSNFFDYWGQGTTVTVSS
>Heavy Chain of hSC16.15 IgG histidine scanning variant #30
(SEQ ID NO: 366)
QVQLVQSGAEVKKPGASVKVSCKASGYTFTRYWIHWIRQAPGQGLEWMGYINPTTVYTEFNQN
FKHRVTMTRDTSTSTVYMELSSLRSEDTAVYYCARGGSNFFDYWGQGTTVTVSS
>Heavy Chain of hSC16.15 IgG histidine scanning variant #31
(SEQ ID NO: 367)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCHRGGSNFFDYWGQGTTVTVSS
>Heavy Chain of hSC16.15 IgG histidine scanning variant #32
(SEQ ID NO: 368)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCAHGGSNFFDYWGQGTTVTVSS
>Heavy Chain of hSC16.15 IgG histidine scanning variant #33
(SEQ ID NO: 369)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARHGSNFFDYWGQGTTVTVSS
>Heavy Chain of hSC16.15 IgG histidine scanning variant #34
(SEQ ID NO: 370)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGHSNFFDYWGQGTTVTVSS
>Heavy Chain of hSC16.15 IgG histidine scanning variant #35
(SEQ ID NO: 371)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGHNFFDYWGQGTTVTVSS
>Heavy Chain of hSC16.15 IgG histidine scanning variant #36
(SEQ ID NO: 372)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSHFFDYWGQGTTVTVSS
>Heavy Chain of hSC16.15 IgG histidine scanning variant #37
(SEQ ID NO: 373)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSNHFDYWGQGTTVTVSS
>Heavy Chain of hSC16.15 IgG histidine scanning variant #38
(SEQ ID NO: 374)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSNFHDYWGQGTTVTVSS
>Heavy Chain of hSC16.15 IgG histidine scanning variant #39
(SEQ ID NO: 375)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSNFFHYWGQGTTVTVSS
>Heavy Chain of hSC16.15 IgG histidine scanning variant #40
(SEQ ID NO: 376)
QVQLVQSGAEVKKPGASVKVSCKASGYTFTRYWIHWIRQAPGQGLEWMGYINPTTVYTEFNQN
FKDRVTMTRDTSTSTVYMELSSLRSEDTAVYYCARGGSNFFDHWGQGTTVTVSS
>Light Chain of hSC16.15 IgG histidine scanning variant #1
(SEQ ID NO: 377)
AIQLTQSPSSLSASVGDRVTITCRASHNIYYNLAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #2
(SEQ ID NO: 378)
AIQLTQSPSSLSASVGDRVTITCRASEHIYYNLAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #3
(SEQ ID NO: 379)
AIQLTQSPSSLSASVGDRVTITCRASENHYYNLAWYQQKPGKAPKLLIYTANSLEDGVPSRFSGS
GSGTDFTLTISSLQPEDFATYFCKQAYDVPPTFGGGTKLEIK
>Light Chain of hSC16.15 IgG histidine scanning variant #4
(SEQ ID NO: 380)
AIQLTQSPSSLSASVGDRVTITCRASENIHYNLAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #5
(SEQ ID NO: 381)
AIQLTQSPSSLSASVGDRVTITCRASENIYHNLAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #6
(SEQ ID NO: 382)
AIQLTQSPSSLSASVGDRVTITCRASENIYYHLAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #7
(SEQ ID NO: 383)
AIQLTQSPSSLSASVGDRVTITCRASENIYYNHAWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #8
(SEQ ID NO: 384)
AIQLTQSPSSLSASVGDRVTITCRASENIYYNLHWYQQKPGKAPKLLIYTANSLEDGVPSRFSGSG
SGTDFTLTISSLQPEDFATYFCKQAYDVPPTFGGGTKLEIK
>Light Chain of hSC16.15 IgG histidine scanning variant #9
(SEQ ID NO: 385)
AIQLTQSPSSLSASVGDRVTITCRASENIYYNLAWYQQKPGKAPKLLIYHANSLEDGVPSRFSGSG
SGTDFTLTISSLQPEDFATYFCKQAYDVPPTFGGGTKLEIK
>Light Chain of hSC16.15 IgG histidine scanning variant #10
(SEQ ID NO: 386)
AIQLTQSPSSLSASVGDRVTITCRASENIYYNLAWYQQKPGKAPKLLIYTHNSLEDGVPSRFSGSG
SGTDFTLTISSLQPEDFATYFCKQAYDVPPTFGGGTKLEIK
>Light Chain of hSC16.15 IgG histidine scanning variant #11
(SEQ ID NO: 387)
AIQLTQSPSSLSASVGDRVTITCRASENIYYNLAWYQQKPGKAPKLLIYTAHSLEDGVPSRFSGSG
SGTDFTLTISSLQPEDFATYFCKQAYDVPPTFGGGTKLEIK
>Light Chain of hSC16.15 IgG histidine scanning variant #12
(SEQ ID NO: 388)
AIQLTQSPSSLSASVGDRVTITCRASENIYYNLAWYQQKPGKAPKWYTANHLEDGVPSRFSGSG
SGTDFTLTISSLQPEDFATYFCKQAYDVPPTFGGGTKLEIK
>Light Chain of hSC16.15 IgG histidine scanning variant #13
(SEQ ID NO: 389)
AIQLTQSPSSLSASVGDRVTITCRASENIYYNLAWYQQKPGKAPKLLIYTANSHEDGVPSRFSGSG
SGTDFTLTISSLQPEDFATYFCKQAYDVPPTFGGGTKLEIK
>Light Chain of hSC16.15 IgG histidine scanning variant #14
(SEQ ID NO: 390)
AIQLTQSPSSLSASVGDRVTITCRASENIYYNLAWYQQKPGKAPKLLIYTANSLHDGVPSRFSGSG
SGTDFTLTISSLQPEDFATYFCKQAYDVPPTFGGGTKLEIK
>Light Chain of hSC16.15 IgG histidine scanning variant #15
(SEQ ID NO: 391)
AIQLTQSPSSLSASVGDRVTITCRASENIYYNLAWYQQKPGKAPKLLIYTANSLEHGVPSRFSGSG
SGTDFTLTISSLQPEDFATYFCKQAYDVPPTFGGGTKLEIK
>Light Chain of hSC16.15 IgG histidine scanning variant #16
(SEQ ID NO: 392)
AIQLTQSPSSLSASVGDRVTITCRASENIYYNLAWYQQKPGKAPKLLIYTANSLEDGVPSRFSGSG
SGTDFTLTISSLQPEDFATYFCHQAYDVPPTFGGGTKLEIK
>Light Chain of hSC16.15 IgG histidine scanning variant #17
(SEQ ID NO: 393)
AIQLTQSPSSLSASVGDRVTITCRASENIYYNLAWYQQKPGKAPKLLIYTANSLEDGVPSRFSGSG
SGTDFTLTISSLQPEDFATYFCKHAYDVPPTFGGGTKLEIK
>Light Chain of hSC16.15 IgG histidine scanning variant #18
(SEQ ID NO: 394)
AIQLTQSPSSLSASVGDRVTITCRASENIYYNLAWYQQKPGKAPKLLIYTANSLEDGVPSRFSGSG
SGTDFTLTISSLQPEDFATYFCKQHYDVPPTFGGGTKLEIK
>Light Chain of hSC16.15 IgG histidine scanning variant #19
(SEQ ID NO: 395)
AIQLTQSPSSLSASVGDRVTITCRASENIYYNLAWYQQKPGKAPKLLIYTANSLEDGVPSRFSGSG
SGTDFTLTISSLQPEDFATYFCKQAHDVPPTFGGGTKLEIK
>Light Chain of hSC16.15 IgG histidine scanning variant #20
(SEQ ID NO: 396)
AIQLTQSPSSLSASVGDRVTITCRASENIYYNLAWYQQKPGKAPKLLIYTANSLEDGVPSRFSGSG
SGTDFTLTISSLQPEDFATYFCKQAYHVPPTFGGGTKLEIK
>Light Chain of hSC16.15 IgG histidine scanning variant #21
(SEQ ID NO: 397)
AIQLTQSPSSLSASVGDRVTITCRASENIYYNLAWYQQKPGKAPKLLIYTANSLEDGVPSRFSGSG
SGTDFTLTISSLQPEDFATYFCKQAYDHPPTFGGGTKLEIK
>Light Chain of hSC16.15 IgG histidine scanning variant #22
(SEQ ID NO: 398)
AIQLTQSPSSLSASVGDRVTITCRASENIYYNLAWYQQKPGKAPKLLIYTANSLEDGVPSRFSGSG
SGTDFTLTISSLQPEDFATYFCKQAYDVHPTFGGGTKLEIK
>Light Chain of hSC16.15 IgG histidine scanning variant #23
(SEQ ID NO: 399)
AIQLTQSPSSLSASVGDRVTITCRASENIYYNLAWYQQKPGKAPKLLIYTANSLEDGVPSRFSGSG
SGTDFTLTISSLQPEDFATYFCKQAYDVPHTFGGGTKLEIK
>Light Chain of hSC16.15 IgG histidine scanning variant #24
(SEQ ID NO: 400)
AIQLTQSPSSLSASVGDRVTITCRASENIYYNLAWYQQKPGKAPKWYTANSLEDGVPSRFSGSG
SGTDFTLTISSLQPEDFATYFCKQAYDVPPHFGGGTKLEIK
>Heavy Chain of hSC16.25 IgG
(SEQ ID NO: 401)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYYAMDYWGQGTTLVTVSS
>Light Chain of hSC16.25 IgG
(SEQ ID NO: 402)
EIVLTQSPDFQSVTPKEKVTITCSASSSVSYMHWYQQKPDQSPKLLIKDSSKLASGVPSRFSGSGS
GTDFTLTINSLEAEDAATYYCQQWSSNPLTFGQGTKLEIK
>Heavy Chain of hSC16.25 IgG histidine scanning variant #1
(SEQ ID NO: 403)
QITLKESGPTLVKPTQTLTLTCTFSHFSLSTSGMGVGWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #2
(SEQ ID NO: 404)
QITLKESGPTLVKPTQTLTLTCTFSGHSLSTSGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #3
(SEQ ID NO: 405)
QITLKESGPTLVKPTQTLTLTCTFSGFHLSTSGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #4
(SEQ ID NO: 406)
QITLKESGPTLVKPTQTLTLTCTFSGFSHSTSGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #5
(SEQ ID NO: 407)
QITLKESGPTLVKPTQTLTLTCTFSGFSLHTSGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #6
(SEQ ID NO: 408)
QITLKESGPTLVKPTQTLTLTCTFSGFSLSHSGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #7
(SEQ ID NO: 409)
QITLKESGPTLVKPTQTLTLTCTFSGFSLSTHGMGVGWIRQPPGKALEWLTDIWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #8
(SEQ ID NO: 410)
QITLKESGPTLVKPTQTLTLTCTFSGFSLSTSHMGVGWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #9
(SEQ ID NO: 411)
QITLKESGPTLVKPTQTLTLTCTFSGFSLSTSGHGVGWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #10
(SEQ ID NO: 412)
QITLKESGPTLVKPTQTLTLTCTFSGFSLSTSGMHVGWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #11
(SEQ ID NO: 413)
QITLKESGPTLVKPTQTLTLTCTFSGFSLSTSGMGHGWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #12
(SEQ ID NO: 414)
QITLKESGPTLVKPTQTLTLTCTFSGFSLSTSGMGVHWIRQPPGKALEWLTDIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #13
(SEQ ID NO: 415)
QITLKESGPTLVKPTQTLTLTCTFSGFSLSTSGMGVGWIRQPPGKALEWLTHIWWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #14
(SEQ ID NO: 416)
QITLKESGPTLVKPTQTLTLTCTFSGFSLSTSGMGVGWIRQPPGKALEWLTDHWWDDNKYYNPS
LKSRLTITKDTSKNQVVLTMTNMDPVDTATYYCARRVNYYYDPYYAMDYWGQGTTLVTVSS
>Heavy Chain of hSC16.25 IgG histidine scanning variant #15
(SEQ ID NO: 417)
QITLKESGPTLVKPTQTLTLTCTFSGFSLSTSGMGVGWIRQPPGKALEWLTDIHWDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #16
(SEQ ID NO: 418)
QITLKESGPTLVKPTQTLTLTCTFSGFSLSTSGMGVGWIRQPPGKALEWLTDIWHDDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #17
(SEQ ID NO: 419)
QITLKESGPTLVKPTQTLTLTCTFSGFSLSTSGMGVGWIRQPPGKALEWLTDIWWHDNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #18
(SEQ ID NO: 420)
QITLKESGPTLVKPTQTLTLTCTFSGFSLSTSGMGVGWIRQPPGKALEWLTDIWWDHNKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #19
(SEQ ID NO: 421)
QITLKESGPTLVKPTQTLTLTCTFSGFSLSTSGMGVGWIRQPPGKALEWLTDIWWDDHKYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #20
(SEQ ID NO: 422)
QITLKESGPTLVKPTQTLTLTCTFSGFSLSTSGMGVGWIRQPPGKALEWLTDIWWDDNHYYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #21
(SEQ ID NO: 423)
QITLKESGPTLVKPTQTLTLTCTFSGFSLSTSGMGVGWIRQPPGKALEWLTDIWWDDNKHYNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #22
(SEQ ID NO: 424)
QITLKESGPTLVKPTQTLTLTCTFSGFSLSTSGMGVGWIRQPPGKALEWLTDIWWDDNKYHNPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #23
(SEQ ID NO: 425)
QITLKESGPTLVKPTQTLTLTCTFSGFSLSTSGMGVGWIRQPPGKALEWLTDIWWDDNKYYHPSL
KSRLTITKDTSKNQVVLTMTNMDPVDTATYYCARRVNYYYDPYYAMDYWGQGTTLVTVSS
>Heavy Chain of hSC16.25 IgG histidine scanning variant #24
(SEQ ID NO: 426)
QITLKESGPTLVKPTQTLTLTCTFSGFSLSTSGMGVGWIRQPPGKALEWLTDIWWDDNKYYNHS
LKSRLTITKDTSKNQVVLTMTNMDPVDTATYYCARRVNYYYDPYYAMDYWGQGTTLVTVSS
>Heavy Chain of hSC16.25 IgG histidine scanning variant #25
(SEQ ID NO: 427)
QITLKESGPTLVKPTQTLTLTCTFSGFSLSTSGMGVGWIRQPPGKALEWLTDIWWDDNKYYNPH
LKSRLTITKDTSKNQVVLTMTNMDPVDTATYYCARRVNYYYDPYYAMDYWGQGTTLVTVSS
>Heavy Chain of hSC16.25 IgG histidine scanning variant #26
(SEQ ID NO: 428)
QITLKESGPTLVKPTQTLTLTCTFSGFSLSTSGMGVGWIRQPPGKALEWLTDIWWDDNKYYNPS
HKSRLTITKDTSKNQVVLTMTNMDPVDTATYYCARRVNYYYDPYYAMDYWGQGTTLVTVSS
>Heavy Chain of hSC16.25 IgG histidine scanning variant #27
(SEQ ID NO: 429)
QITLKESGPTLVKPTQTLTLTCTFSGFSLSTSGMGVGWIRQPPGKALEWLTDIWWDDNKYYNPSL
HSRLTITKDTSKNQVVLTMTNMDPVDTATYYCARRVNYYYDPYYAMDYWGQGTTLVTVSS
>Heavy Chain of hSC16.25 IgG histidine scanning variant #28
(SEQ ID NO: 430)
QITLKESGPTLVKPTQTLTLTCTFSGFSLSTSGMGVGWIRQPPGKALEWLTDIWWDDNKYYNPSL
KHRLTITKDTSKNQVVLTMTNMDPVDTATYYCARRVNYYYDPYYAMDYWGQGTTLVTVSS
>Heavy Chain of hSC16.25 IgG histidine scanning variant #29
(SEQ ID NO: 431)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCHRRVNYYYDPYYAMDYWGQGTTLVTVSS
>Heavy Chain of hSC16.25 IgG histidine scanning variant #30
(SEQ ID NO: 432)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCAHRVNYYYDPYYAMDYWGQGTTLVTVSS
>Heavy Chain of hSC16.25 IgG histidine scanning variant #31
(SEQ ID NO: 433)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARHVNYYYDPYYAMDYWGQGTTLVTVSS
>Heavy Chain of hSC16.25 IgG histidine scanning variant #32
(SEQ ID NO: 434)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRHNYYYDPYYAMDYWGQGTTLVTVSS
>Heavy Chain of hSC16.25 IgG histidine scanning variant #33
(SEQ ID NO: 435)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVHYYYDPYYAMDYWGQGTTLVTVSS
>Heavy Chain of hSC16.25 IgG histidine scanning variant #34
(SEQ ID NO: 436)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNHYYDPYYAMDYWGQGTTLVTVSS
>Heavy Chain of hSC16.25 IgG histidine scanning variant #35
(SEQ ID NO: 437)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYHYDPYYAMDYWGQGTTLVTVSS
>Heavy Chain of hSC16.25 IgG histidine scanning variant #36
(SEQ ID NO: 438)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYHDPYYAMDYWGQGTTLVTVSS
>Heavy Chain of hSC16.25 IgG histidine scanning variant #37
(SEQ ID NO: 439)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYHPYYAMDYWGQGTTLVTVSS
>Heavy Chain of hSC16.25 IgG histidine scanning variant #38
(SEQ ID NO: 440)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDHYYAMDYWGQGTTLVTVSS
>Heavy Chain of hSC16.25 IgG histidine scanning variant #39
(SEQ ID NO: 441)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPHYAMDYWGQGTTLVTVSS
>Heavy Chain of hSC16.25 IgG histidine scanning variant #40
(SEQ ID NO: 442)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYHAMDYWGQGTTLVTVSS
>Heavy Chain of hSC16.25 IgG histidine scanning variant #41
(SEQ ID NO: 443)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYYHMDYWGQGTTLVTVSS
>Heavy Chain of hSC16.25 IgG histidine scanning variant #42
(SEQ ID NO: 444)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYYAHDYWGQGTTLVTVSS
>Heavy Chain of hSC16.25 IgG histidine scanning variant #43
(SEQ ID NO: 445)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYYAMHYWGQGTTLVTVSS
>Heavy Chain of hSC16.25 IgG histidine scanning variant #44
(SEQ ID NO: 446)
QITLKESGPTLVKPTQTLTLTCTFSGFSLSTSGMGVGWIRQPPGKALEWLTDIWWDDNKYYNPSL
KSRLTITKDTSKNQVVLTMTNMDPVDTATYYCARRVNYYYDPYYAMDHWGQGTTLVTVSS
>Light Chain of hSC16.25 IgG histidine scanning variant #1
(SEQ ID NO: 447)
EIVLTQSPDFQSVTPKEKVTITCSASHSVSYMHWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #2
(SEQ ID NO: 448)
EIVLTQSPDFQSVTPKEKVTITCSASSHVSYMHWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #3
(SEQ ID NO: 449)
EIVLTQSPDFQSVTPKEKVTITCSASSSHSYMEIWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #4
(SEQ ID NO: 450)
EIVLTQSPDFQSVTPKEKVTITCSASSSVHYMHWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #5
(SEQ ID NO: 451)
EIVLTQSPDFQSVTPKEKVTITCSASSSVSHMEIWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #6
(SEQ ID NO: 452)
EIVLTQSPDFQSVTPKEKVTITCSASSSVSYHEIWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #7
(SEQ ID NO: 453)
EIVLTQSPDFQSVTPKEKVTITCSASSSVSYMAWYQQKPDQSPKLLIKDSSKLASGVPSRFSGSGS
GTDFTLTINSLEAEDAATYYCQQWSSNPLTFGQGTKLEIK
>Light Chain of hSC16.25 IgG histidine scanning variant #8
(SEQ ID NO: 454)
EIVLTQSPDFQSVTPKEKVTITCSASSSVSYMHWYQQKPDQSPKLLIKHSSKLASGVPSRFSGSGS
GTDFTLTINSLEAEDAATYYCQQWSSNPLTFGQGTKLEIK
>Light Chain of hSC16.25 IgG histidine scanning variant #9
(SEQ ID NO: 455)
EIVLTQSPDFQSVTPKEKVTITCSASSSVSYMHWYQQKPDQSPKLLIKDHSKLASGVPSRFSGSGS
GTDFTLTINSLEAEDAATYYCQQWSSNPLTFGQGTKLEIK
>Light Chain of hSC16.25 IgG histidine scanning variant #10
(SEQ ID NO: 456)
EIVLTQSPDFQSVTPKEKVTITCSASSSVSYMHWYQQKPDQSPKLLIKDSHKLASGVPSRFSGSGS
GTDFTLTINSLEAEDAATYYCQQWSSNPLTFGQGTKLEIK
>Light Chain of hSC16.25 IgG histidine scanning variant #11
(SEQ ID NO: 457)
EIVLTQSPDFQSVTPKEKVTITCSASSSVSYMHWYQQKPDQSPKLLIKDSSHLASGVPSRFSGSGS
GTDFTLTINSLEAEDAATYYCQQWSSNPLTFGQGTKLEIK
>Light Chain of hSC16.25 IgG histidine scanning variant #12
(SEQ ID NO: 458)
EIVLTQSPDFQSVTPKEKVTITCSASSSVSYMHWYQQKPDQSPKLLIKDSSKHASGVPSRFSGSGS
GTDFTLTINSLEAEDAATYYCQQWSSNPLTFGQGTKLEIK
>Light Chain of hSC16.25 IgG histidine scanning variant #13
(SEQ ID NO: 459)
EIVLTQSPDFQSVTPKEKVTITCSASSSVSYMHWYQQKPDQSPKLLIKDSSKLHSGVPSRFSGSGS
GTDFTLTINSLEAEDAATYYCQQWSSNPLTFGQGTKLEIK
>Light Chain of hSC16.25 IgG histidine scanning variant #14
(SEQ ID NO: 460)
EIVLTQSPDFQSVTPKEKVTITCSASSSVSYMHWYQQKPDQSPKLLIKDSSKLAHGVPSRFSGSGS
GTDFTLTINSLEAEDAATYYCQQWSSNPLTFGQGTKLEIK
>Light Chain of hSC16.25 IgG histidine scanning variant #15
(SEQ ID NO: 461)
EIVLTQSPDFQSVTPKEKVTITCSASSSVSYMHWYQQKPDQSPKLLIKDSSKLASGVPSRFSGSGS
GTDFTLTINSLEAEDAATYYCHQWSSNPLTFGQGTKLEIK
>Light Chain of hSC16.25 IgG histidine scanning variant #16
(SEQ ID NO: 462)
EIVLTQSPDFQSVTPKEKVTITCSASSSVSYMHWYQQKPDQSPKLLIKDSSKLASGVPSRFSGSGS
GTDFTLTINSLEAEDAATYYCQHWSSNPLTFGQGTKLEIK
>Light Chain of hSC16.25 IgG histidine scanning variant #17
(SEQ ID NO: 463)
EIVLTQSPDFQSVTPKEKVTITCSASSSVSYMEIWYQQKPDQSPKLLIKDSSKLASGVPSRFSGSGS
GTDFTLTINSLEAEDAATYYCQQHSSNPLTFGQGTKLEIK
>Light Chain of hSC16.25 IgG histidine scanning variant #18
(SEQ ID NO: 464)
EIVLTQSPDFQSVTPKEKVTITCSASSSVSYMEIWYQQKPDQSPKLLIKDSSKLASGVPSRFSGSGS
GTDFTLTINSLEAEDAATYYCQQWHSNPLTFGQGTKLEIK
>Light Chain of hSC16.25 IgG histidine scanning variant #19
(SEQ ID NO: 465)
EIVLTQSPDFQSVTPKEKVTITCSASSSVSYMEIWYQQKPDQSPKLLIKDSSKLASGVPSRFSGSGS
GTDFTLTINSLEAEDAATYYCQQWSHNPLTFGQGTKLEIK
>Light Chain of hSC16.25 IgG histidine scanning variant #20
(SEQ ID NO: 466)
EIVLTQSPDFQSVTPKEKVTITCSASSSVSYMEIWYQQKPDQSPKLLIKDSSKLASGVPSRFSGSGS
GTDFTLTINSLEAEDAATYYCQQWSSHPLTFGQGTKLEIK
>Light Chain of hSC16.25 IgG histidine scanning variant #21
(SEQ ID NO: 467)
EIVLTQSPDFQSVTPKEKVTITCSASSSVSYMEIWYQQKPDQSPKLLIKDSSKLASGVPSRFSGSGS
GTDFTLTINSLEAEDAATYYCQQWSSNHLTFGQGTKLEIK
>Light Chain of hSC16.25 IgG histidine scanning variant #22
(SEQ ID NO: 468)
EIVLTQSPDFQSVTPKEKVTITCSASSSVSYMEIWYQQKPDQSPKLLIKDSSKLASGVPSRFSGSGS
GTDFTLTINSLEAEDAATYYCQQWSSNPHTFGQGTKLEIK
>Light Chain of hSC16.25 IgG histidine scanning variant #23
(SEQ ID NO: 469)
EIVLTQSPDFQSVTPKEKVTITCSASSSVSYMEIWYQQKPDQSPKLLIKDSSKLASGVPSRFSGSGS
GTDFTLTINSLEAEDAATYYCQQWSSNPLHFGQGTKLEIK
>Heavy Chain of hSC16.34 IgG
(SEQ ID NO: 470)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Light Chain of hSC16.34 IgG
(SEQ ID NO: 471)
DIQMTQSPSSLSASVGDRVTITCKASQSVSNDVAWYQQKPGKVPKLLIYYASNRYSGVPSRFSGS
GSGTDFTLTISSLQPEDVATYFCQQDYSSPWTFGGGTKVEIK
>Heavy Chain of hSC16.34 IgG histidine scanning variant #1
(SEQ ID NO: 472)
QVQLVQSGAEVKKPGASVKVSCHASGYTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #2
(SEQ ID NO: 473)
QVQLVQSGAEVKKPGASVKVSCKHSGYTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #3
(SEQ ID NO: 474)
QVQLVQSGAEVKKPGASVKVSCKAHGYTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #4
(SEQ ID NO: 475)
QVQLVQSGAEVKKPGASVKVSCKASHYTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #5
(SEQ ID NO: 476)
QVQLVQSGAEVKKPGASVKVSCKASGHTFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #6
(SEQ ID NO: 477)
QVQLVQSGAEVKKPGASVKVSCKASGYHFTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #7
(SEQ ID NO: 478)
QVQLVQSGAEVKKPGASVKVSCKASGYTHTNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #8
(SEQ ID NO: 479)
QVQLVQSGAEVKKPGASVKVSCKASGYTFHNYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #9
(SEQ ID NO: 480)
QVQLVQSGAEVKKPGASVKVSCKASGYTFTHYGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #10
(SEQ ID NO: 481)
QVQLVQSGAEVKKPGASVKVSCKASGYTFTNHGMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #11
(SEQ ID NO: 482)
QVQLVQSGAEVKKPGASVKVSCKASGYTFTNYHMNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #12
(SEQ ID NO: 483)
QVQLVQSGAEVKKPGASVKVSCKASGYTFTNYGHNWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #13
(SEQ ID NO: 484)
QVQLVQSGAEVKKPGASVKVSCKASGYTFTNYGMHWVRQAPGQRLEWMGWINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #14
(SEQ ID NO: 485)
QVQLVQSGAEVKKPGASVKVSCKASGYTFTNYGMNWVRQAPGQRLEWMGHINTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #15
(SEQ ID NO: 486)
QVQLVQSGAEVKKPGASVKVSCKASGYTFTNYGMNWVRQAPGQRLEWMGWHNTYTGDPTY
ADDFKGRVTITRDTSASTAYMELSSLRSEDTAVYYCARIGGNSPSDYWGQGTTVTVSS
>Heavy Chain of hSC16.34 IgG histidine scanning variant #16
(SEQ ID NO: 487)
QVQLVQSGAEVKKPGASVKVSCKASGYTFTNYGMNWVRQAPGQRLEWMGWIHTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #17
(SEQ ID NO: 488)
QVQLVQSGAEVKKPGASVKVSCKASGYTFTNYGMNWVRQAPGQRLEWMGWIHTYTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #18
(SEQ ID NO: 489)
QVQLVQSGAEVKKPGASVKVSCKASGYTFTNYGMNWVRQAPGQRLEWMGWINTHTGDPTYA
DDFKGRVTITRDTSASTAYMELSSLRSEDTAVYYCARIGGNSPSDYWGQGTTVTVSS
>Heavy Chain of hSC16.34 IgG histidine scanning variant #19
(SEQ ID NO: 490)
QVQLVQSGAEVKKPGASVKVSCKASGYTFTNYGMNWVRQAPGQRLEWMGWINTYHGDPTYA
DDFKGRVTITRDTSASTAYMELSSLRSEDTAVYYCARIGGNSPSDYWGQGTTVTVSS
>Heavy Chain of hSC16.34 IgG histidine scanning variant #20
(SEQ ID NO: 491)
QVQLVQSGAEVKKPGASVKVSCKASGYTFTNYGMNWVRQAPGQRLEWMGWINTYTHDPTYA
DDFKGRVTITRDTSASTAYMELSSLRSEDTAVYYCARIGGNSPSDYWGQGTTVTVSS
>Heavy Chain of hSC16.34 IgG histidine scanning variant #21
(SEQ ID NO: 492)
QVQLVQSGAEVKKPGASVKVSCKASGYTFTNYGMNWVRQAPGQRLEWMGWINTYTGHPTYA
DDFKGRVTITRDTSASTAYMELSSLRSEDTAVYYCARIGGNSPSDYWGQGTTVTVSS
>Heavy Chain of hSC16.34 IgG histidine scanning variant #22
(SEQ ID NO: 493)
QVQLVQSGAEVKKPGASVKVSCKASGYTFTNYGMNWVRQAPGQRLEWMGWINTYTGDHTYA
DDFKGRVTITRDTSASTAYMELSSLRSEDTAVYYCARIGGNSPSDYWGQGTTVTVSS
>Heavy Chain of hSC16.34 IgG histidine scanning variant #23
(SEQ ID NO: 494)
QVQLVQSGAEVKKPGASVKVSCKASGYTFTNYGMNWVRQAPGQRLEWMGWINTYTGDPHYA
DDFKGRVTITRDTSASTAYMELSSLRSEDTAVYYCARIGGNSPSDYWGQGTTVTVSS
>Heavy Chain of hSC16.34 IgG histidine scanning variant #24
(SEQ ID NO: 495)
QVQLVQSGAEVKKPGASVKVSCKASGYTFTNYGMNWVRQAPGQRLEWMGWINTYTGDPTHA
DDFKGRVTITRDTSASTAYMELSSLRSEDTAVYYCARIGGNSPSDYWGQGTTVTVSS
>Heavy Chain of hSC16.34 IgG histidine scanning variant #25
(SEQ ID NO: 496)
QVQLVQSGAEVKKPGASVKVSCKASGYTFTNYGMNWVRQAPGQRLEWMGWINTYTGDPTYH
DDFKGRVTITRDTSASTAYMELSSLRSEDTAVYYCARIGGNSPSDYWGQGTTVTVSS
>Heavy Chain of hSC16.34 IgG histidine scanning variant #26
(SEQ ID NO: 497)
QVQLVQSGAEVKKPGASVKVSCKASGYTFTNYGMNWVRQAPGQRLEWMGWINTYTGDPTYA
HDFKGRVTITRDTSASTAYMELSSLRSEDTAVYYCARIGGNSPSDYWGQGTTVTVSS
>Heavy Chain of hSC16.34 IgG histidine scanning variant #27
(SEQ ID NO: 498)
QVQLVQSGAEVKKPGASVKVSCKASGYTFTNYGMNWVRQAPGQRLEWMGWINTYTGDPTYA
DHFKGRVTITRDTSASTAYMELSSLRSEDTAVYYCARIGGNSPSDYWGQGTTVTVSS
>Heavy Chain of hSC16.34 IgG histidine scanning variant #28
(SEQ ID NO: 499)
QVQLVQSGAEVKKPGASVKVSCKASGYTFTNYGMNWVRQAPGQRLEWMGWINTYTGDPTYA
DDHKGRVTITRDTSASTAYMELSSLRSEDTAVYYCARIGGNSPSDYWGQGTTVTVSS
>Heavy Chain of hSC16.34 IgG histidine scanning variant #29
(SEQ ID NO: 500)
QVQLVQSGAEVKKPGASVKVSCKASGYTFTNYGMNWVRQAPGQRLEWMGWINTYTGDPTYA
DDFHGRVTITRDTSASTAYMELSSLRSEDTAVYYCARIGGNSPSDYWGQGTTVTVSS
>Heavy Chain of hSC16.34 IgG histidine scanning variant #30
(SEQ ID NO: 501)
QVQLVQSGAEVKKPGASVKVSCKASGYTFTNYGMNWVRQAPGQRLEWMGWINTYTGDPTYA
DDFKHRVTITRDTSASTAYMELSSLRSEDTAVYYCARIGGNSPSDYWGQGTTVTVSS
>Heavy Chain of hSC16.34 IgG histidine scanning variant #31
(SEQ ID NO: 502)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCHRIGGNSPSDYWGQGTTVTVSS
>Heavy Chain of hSC16.34 IgG histidine scanning variant #32
(SEQ ID NO: 503)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCAHIGGNSPSDYWGQGTTVTVSS
>Heavy Chain of hSC16.34 IgG histidine scanning variant #33
(SEQ ID NO: 504)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARHGGNSPSDYWGQGTTVTVSS
>Heavy Chain of hSC16.34 IgG histidine scanning variant #34
(SEQ ID NO: 505)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIHGNSPSDYWGQGTTVTVSS
>Heavy Chain of hSC16.34 IgG histidine scanning variant #35
(SEQ ID NO: 506)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGHNSPSDYWGQGTTVTVSS
>Heavy Chain of hSC16.34 IgG histidine scanning variant #36
(SEQ ID NO: 507)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGHSPSDYWGQGTTVTVSS
>Heavy Chain of hSC16.34 IgG histidine scanning variant #37
(SEQ ID NO: 508)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNHPSDYWGQGTTVTVSS
>Heavy Chain of hSC16.34 IgG histidine scanning variant #38
(SEQ ID NO: 509)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNSHSDYWGQGTTVTVSS
>Heavy Chain of hSC16.34 IgG histidine scanning variant #39
(SEQ ID NO: 510)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNSPHDYWGQGTTVTVSS
>Heavy Chain of hSC16.34 IgG histidine scanning variant #40
(SEQ ID NO: 511)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNSPSHYWGQGTTVTVSS
>Heavy Chain of hSC16.34 IgG histidine scanning variant #41
(SEQ ID NO: 512)
QVQLVQSGAEVKKPGASVKVSCKASGYTFTNYGMNWVRQAPGQRLEWMGWINTYTGDPTYA
DDFKGRVTITRDTSASTAYMELSSLRSEDTAVYYCARIGGNSPSDHWGQGTTVTVSS
>Light Chain of hSC16.34 IgG histidine scanning variant #1
(SEQ ID NO: 513)
DIQMTQSPSSLSASVGDRVTITCKASHSVSNDVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #2
(SEQ ID NO: 514)
DIQMTQSPSSLSASVGDRVTITCKASQHVSNDVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #3
(SEQ ID NO: 515)
DIQMTQSPSSLSASVGDRVTITCKASQSHSNDVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #4
(SEQ ID NO: 516)
DIQMTQSPSSLSASVGDRVTITCKASQSVHNDVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #5
(SEQ ID NO: 517)
DIQMTQSPSSLSASVGDRVTITCKASQSVSHDVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #6
(SEQ ID NO: 518)
DIQMTQSPSSLSASVGDRVTITCKASQSVSNHVAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #7
(SEQ ID NO: 519)
DIQMTQSPSSLSASVGDRVTITCKASQSVSNDHAWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #8
(SEQ ID NO: 520)
DIQMTQSPSSLSASVGDRVTITCKASQSVSNDVHWYQQKPGKVPKLLIYYASNRYSGVPSRFSGS
GSGTDFTLTISSLQPEDVATYFCQQDYSSPWTFGGGTKVEIK
>Light Chain of hSC16.34 IgG histidine scanning variant #9
(SEQ ID NO: 521)
DIQMTQSPSSLSASVGDRVTITCKASQSVSNDVAWYQQKPGKVPKLLIYHASNRYSGVPSRFSGS
GSGTDFTLTISSLQPEDVATYFCQQDYSSPWTFGGGTKVEIK
>Light Chain of hSC16.34 IgG histidine scanning variant #10
(SEQ ID NO: 522)
DIQMTQSPSSLSASVGDRVTITCKASQSVSNDVAWYQQKPGKVPKLLIYYHSNRYSGVPSRFSGS
GSGTDFTLTISSLQPEDVATYFCQQDYSSPWTFGGGTKVEIK
>Light Chain of hSC16.34 IgG histidine scanning variant #11
(SEQ ID NO: 523)
DIQMTQSPSSLSASVGDRVTITCKASQSVSNDVAWYQQKPGKVPKLLIYYAHNRYSGVPSRFSGS
GSGTDFTLTISSLQPEDVATYFCQQDYSSPWTFGGGTKVEIK
>Light Chain of hSC16.34 IgG histidine scanning variant #12
(SEQ ID NO: 524)
DIQMTQSPSSLSASVGDRVTITCKASQSVSNDVAWYQQKPGKVPKLLIYYASHRYSGVPSRFSGS
GSGTDFTLTISSLQPEDVATYFCQQDYSSPWTFGGGTKVEIK
>Light Chain of hSC16.34 IgG histidine scanning variant #13
(SEQ ID NO: 525)
DIQMTQSPSSLSASVGDRVTITCKASQSVSNDVAWYQQKPGKVPKLLIYYASNHYSGVPSRFSGS
GSGTDFTLTISSLQPEDVATYFCQQDYSSPWTFGGGTKVEIK
>Light Chain of hSC16.34 IgG histidine scanning variant #14
(SEQ ID NO: 526)
DIQMTQSPSSLSASVGDRVTITCKASQSVSNDVAWYQQKPGKVPKLLIYYASNRHSGVPSRFSGS
GSGTDFTLTISSLQPEDVATYFCQQDYSSPWTFGGGTKVEIK
>Light Chain of hSC16.34 IgG histidine scanning variant #15
(SEQ ID NO: 527)
DIQMTQSPSSLSASVGDRVTITCKASQSVSNDVAWYQQKPGKVPKLLIYYASNRYHGVPSRFSGS
GSGTDFTLTISSLQPEDVATYFCQQDYSSPWTFGGGTKVEIK
>Light Chain of hSC16.34 IgG histidine scanning variant #16
(SEQ ID NO: 528)
DIQMTQSPSSLSASVGDRVTITCKASQSVSNDVAWYQQKPGKVPKLLIYYASNRYSGVPSRFSGS
GSGTDFTLTISSLQPEDVATYFCHQDYSSPWTFGGGTKVEIK
>Light Chain of hSC16.34 IgG histidine scanning variant #17
(SEQ ID NO: 529)
DIQMTQSPSSLSASVGDRVTITCKASQSVSNDVAWYQQKPGKVPKLLIYYASNRYSGVPSRFSGS
GSGTDFTLTISSLQPEDVATYFCQHDYSSPWTFGGGTKVEIK
>Light Chain of hSC16.34 IgG histidine scanning variant #18
(SEQ ID NO: 530)
DIQMTQSPSSLSASVGDRVTITCKASQSVSNDVAWYQQKPGKVPKLLIYYASNRYSGVPSRFSGS
GSGTDFTLTISSLQPEDVATYFCQQHYSSPWTFGGGTKVEIK
>Light Chain of hSC16.34 IgG histidine scanning variant #19
(SEQ ID NO: 531)
DIQMTQSPSSLSASVGDRVTITCKASQSVSNDVAWYQQKPGKVPKLLIYYASNRYSGVPSRFSGS
GSGTDFTLTISSLQPEDVATYFCQQDHSSPWTFGGGTKVEIK
>Light Chain of hSC16.34 IgG histidine scanning variant #20
(SEQ ID NO: 532)
DIQMTQSPSSLSASVGDRVTITCKASQSVSNDVAWYQQKPGKVPKLLIYYASNRYSGVPSRFSGS
GSGTDFTLTISSLQPEDVATYFCQQDYHSPWTFGGGTKVEIK
>Light Chain of hSC16.34 IgG histidine scanning variant #21
(SEQ ID NO: 533)
DIQMTQSPSSLSASVGDRVTITCKASQSVSNDVAWYQQKPGKVPKLLIYYASNRYSGVPSRFSGS
GSGTDFTLTISSLQPEDVATYFCQQDYSHPWTFGGGTKVEIK
>Light Chain of hSC16.34 IgG histidine scanning variant #22
(SEQ ID NO: 534)
DIQMTQSPSSLSASVGDRVTITCKASQSVSNDVAWYQQKPGKVPKLLIYYASNRYSGVPSRFSGS
GSGTDFTLTISSLQPEDVATYFCQQDYSSHWTFGGGTKVEIK
>Light Chain of hSC16.34 IgG histidine scanning variant #23
(SEQ ID NO: 535)
DIQMTQSPSSLSASVGDRVTITCKASQSVSNDVAWYQQKPGKVPKLLIYYASNRYSGVPSRFSGS
GSGTDFTLTISSLQPEDVATYFCQQDYSSPHTFGGGTKVEIK
>Light Chain of hSC16.34 IgG histidine scanning variant #24
(SEQ ID NO: 536)
DIQMTQSPSSLSASVGDRVTITCKASQSVSNDVAWYQQKPGKVPKLLIYYASNRYSGVPSRFSGS
GSGTDFTLTISSLQPEDVATYFCQQDYSSPWHFGGGTKVEIK
>Heavy Chain of SC16.67 IgG
(SEQ ID NO: 537)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYYDYVYWGQGTLVTVSA
>Light Chain of SC16.67 IgG
(SEQ ID NO: 538)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYSNHLVFGGGTKLTVL
>Heavy Chain of SC16.67 IgG histidine scanning variant #1
(SEQ ID NO: 539)
EVQLVETGGGLVQPKGSLKLSCAVSHFTFTTYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #2
(SEQ ID NO: 540)
EVQLVETGGGLVQPKGSLKLSCAVSAHTFTTYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #3
(SEQ ID NO: 541)
EVQLVETGGGLVQPKGSLKLSCAVSAFHFTTYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #4
(SEQ ID NO: 542)
EVQLVETGGGLVQPKGSLKLSCAVSAFTHTTYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #5
(SEQ ID NO: 543)
EVQLVETGGGLVQPKGSLKLSCAVSAFTFHTYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #6
(SEQ ID NO: 544)
EVQLVETGGGLVQPKGSLKLSCAVSAFTFTHYAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #7
(SEQ ID NO: 545)
EVQLVETGGGLVQPKGSLKLSCAVSAFTFTTHAMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #8
(SEQ ID NO: 546)
EVQLVETGGGLVQPKGSLKLSCAVSAFTFTTYHMNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #9
(SEQ ID NO: 547)
EVQLVETGGGLVQPKGSLKLSCAVSAFTFTTYAHNWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #10
(SEQ ID NO: 548)
EVQLVETGGGLVQPKGSLKLSCAVSAFTFTTYAMHWVRQAPGKGLEWVARIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #11
(SEQ ID NO: 549)
EVQLVETGGGLVQPKGSLKLSCAVSAFTFTTYAMNWVRQAPGKGLEWVAHIRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #12
(SEQ ID NO: 550)
EVQLVETGGGLVQPKGSLKLSCAVSAFTFTTYAMNWVRQAPGKGLEWVARHRNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #13
(SEQ ID NO: 551)
EVQLVETGGGLVQPKGSLKLSCAVSAFTFTTYAMNWVRQAPGKGLEWVARIHNKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #14
(SEQ ID NO: 552)
EVQLVETGGGLVQPKGSLKLSCAVSAFTFTTYAMNWVRQAPGKGLEWVARIRHKSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #15
(SEQ ID NO: 553)
EVQLVETGGGLVQPKGSLKLSCAVSAFTFTTYAMNWVRQAPGKGLEWVARIRNHSNNYATYY
ADSVKDRFTISRDDSQSMLYLQMNNLKIEDTAMYYCVFYYDYVYWGQGTLVTVSA
>Heavy Chain of SC16.67 IgG histidine scanning variant #16
(SEQ ID NO: 554)
EVQLVETGGGLVQPKGSLKLSCAVSAFTFTTYAMNWVRQAPGKGLEWVARIRNKHNNYATYY
ADSVKDRFTISRDDSQSMLYLQMNNLKIEDTAMYYCVFYYDYVYWGQGTLVTVSA
>Heavy Chain of SC16.67 IgG histidine scanning variant #17
(SEQ ID NO: 555)
EVQLVETGGGLVQPKGSLKLSCAVSAFTFTTYAMNWVRQAPGKGLEWVARIRNKSHNYATYY
ADSVKDRFTISRDDSQSMLYLQMNNLKIEDTAMYYCVFYYDYVYWGQGTLVTVSA
>Heavy Chain of SC16.67 IgG histidine scanning variant #18
(SEQ ID NO: 556)
EVQLVETGGGLVQPKGSLKLSCAVSAFTFTTYAMNWVRQAPGKGLEWVARIRNKSNHYATYY
ADSVKDRFTISRDDSQSMLYLQMNNLKIEDTAMYYCVFYYDYVYWGQGTLVTVSA
>Heavy Chain of SC16.67 IgG histidine scanning variant #19
(SEQ ID NO: 557)
EVQLVETGGGLVQPKGSLKLSCAVSAFTFTTYAMNWVRQAPGKGLEWVARIRNKSNNHATYY
ADSVKDRFTISRDDSQSMLYLQMNNLKIEDTAMYYCVFYYDYVYWGQGTLVTVSA
>Heavy Chain of SC16.67 IgG histidine scanning variant #20
(SEQ ID NO: 558)
EVQLVETGGGLVQPKGSLKLSCAVSAFTFTTYAMNWVRQAPGKGLEWVARIRNKSNNYHTYY
ADSVKDRFTISRDDSQSMLYLQMNNLKIEDTAMYYCVFYYDYVYWGQGTLVTVSA
>Heavy Chain of SC16.67 IgG histidine scanning variant #21
(SEQ ID NO: 559)
EVQLVETGGGLVQPKGSLKLSCAVSAFTFTTYAMNWVRQAPGKGLEWVARIRNKSNNYAHYY
ADSVKDRFTISRDDSQSMLYLQMNNLKIEDTAMYYCVFYYDYVYWGQGTLVTVSA
>Heavy Chain of SC16.67 IgG histidine scanning variant #22
(SEQ ID NO: 560)
EVQLVETGGGLVQPKGSLKLSCAVSAFTFTTYAMNWVRQAPGKGLEWVARIRNKSNNYATHY
ADSVKDRFTISRDDSQSMLYLQMNNLKIEDTAMYYCVFYYDYVYWGQGTLVTVSA
>Heavy Chain of SC16.67 IgG histidine scanning variant #23
(SEQ ID NO: 561)
EVQLVETGGGLVQPKGSLKLSCAVSAFTFTTYAMNWVRQAPGKGLEWVARIRNKSNNYATYH
ADSVKDRFTISRDDSQSMLYLQMNNLKIEDTAMYYCVFYYDYVYWGQGTLVTVSA
>Heavy Chain of SC16.67 IgG histidine scanning variant #24
(SEQ ID NO: 562)
EVQLVETGGGLVQPKGSLKLSCAVSAFTFTTYAMNWVRQAPGKGLEWVARIRNKSNNYATYY
HDSVKDRFTISRDDSQSMLYLQMNNLKIEDTAMYYCVFYYDYVYWGQGTLVTVSA
>Heavy Chain of SC16.67 IgG histidine scanning variant #25
(SEQ ID NO: 563)
EVQLVETGGGLVQPKGSLKLSCAVSAFTFTTYAMNWVRQAPGKGLEWVARIRNKSNNYATYY
AHSVKDRFTISRDDSQSMLYLQMNNLKIEDTAMYYCVFYYDYVYWGQGTLVTVSA
>Heavy Chain of SC16.67 IgG histidine scanning variant #26
(SEQ ID NO: 564)
EVQLVETGGGLVQPKGSLKLSCAVSAFTFTTYAMNWVRQAPGKGLEWVARIRNKSNNYATYY
ADHVKDRFTISRDDSQSMLYLQMNNLKIEDTAMYYCVFYYDYVYWGQGTLVTVSA
>Heavy Chain of SC16.67 IgG histidine scanning variant #27
(SEQ ID NO: 565)
EVQLVETGGGLVQPKGSLKLSCAVSAFTFTTYAMNWVRQAPGKGLEWVARIRNKSNNYATYY
ADSHKDRFTISRDDSQSMLYLQMNNLKIEDTAMYYCVFYYDYVYWGQGTLVTVSA
>Heavy Chain of SC16.67 IgG histidine scanning variant #28
(SEQ ID NO: 566)
EVQLVETGGGLVQPKGSLKLSCAVSAFTFTTYAMNWVRQAPGKGLEWVARIRNKSNNYATYY
ADSVHDRFTISRDDSQSMLYLQMNNLKIEDTAMYYCVFYYDYVYWGQGTLVTVSA
>Heavy Chain of SC16.67 IgG histidine scanning variant #29
(SEQ ID NO: 567)
EVQLVETGGGLVQPKGSLKLSCAVSAFTFTTYAMNWVRQAPGKGLEWVARIRNKSNNYATYY
ADSVKHRFTISRDDSQSMLYLQMNNLKIEDTAMYYCVFYYDYVYWGQGTLVTVSA
>Heavy Chain of SC16.67 IgG histidine scanning variant #30
(SEQ ID NO: 568)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCHFYYDYVYWGQGTLVTVSA
>Heavy Chain of SC16.67 IgG histidine scanning variant #31
(SEQ ID NO: 569)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVHYYDYVYWGQGTLVTVSA
>Heavy Chain of SC16.67 IgG histidine scanning variant #32
(SEQ ID NO: 570)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFHYDYVYWGQGTLVTVSA
>Heavy Chain of SC16.67 IgG histidine scanning variant #33
(SEQ ID NO: 571)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYHDYVYWGQGTLVTVSA
>Heavy Chain of SC16.67 IgG histidine scanning variant #34
(SEQ ID NO: 572)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYYHYVYWGQGTLVTVSA
>Heavy Chain of SC16.67 IgG histidine scanning variant #35
(SEQ ID NO: 573)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYYDHVYWGQGTLVTVSA
>Heavy Chain of SC16.67 IgG histidine scanning variant #36
(SEQ ID NO: 574)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYYDYHYWGQGTLVTVSA
>Heavy Chain of SC16.67 IgG histidine scanning variant #37
(SEQ ID NO: 575)
EVQLVETGGGLVQPKGSLKLSCAVSAFTFTTYAMNWVRQAPGKGLEWVARIRNKSNNYATYY
ADSVKDRFTISRDDSQSMLYLQMNNLKIEDTAMYYCVFYYDYVHWGQGTLVTVSA
>Light Chain of SC16.67 IgG histidine scanning variant #1
(SEQ ID NO: 576)
QAVVTQESALTTSPGETVTLTCHSSTGAVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #2
(SEQ ID NO: 577)
QAVVTQESALTTSPGETVTLTCRHSTGAVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #3
(SEQ ID NO: 578)
QAVVTQESALTTSPGETVTLTCRSHTGAVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #4
(SEQ ID NO: 579)
QAVVTQESALTTSPGETVTLTCRSSHGAVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #5
(SEQ ID NO: 580)
QAVVTQESALTTSPGETVTLTCRSSTHAVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #6
(SEQ ID NO: 581)
QAVVTQESALTTSPGETVTLTCRSSTGHVTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #7
(SEQ ID NO: 582)
QAVVTQESALTTSPGETVTLTCRSSTGAHTTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #8
(SEQ ID NO: 583)
QAVVTQESALTTSPGETVTLTCRSSTGAVHTSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #9
(SEQ ID NO: 584)
QAVVTQESALTTSPGETVTLTCRSSTGAVTHSNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #10
(SEQ ID NO: 585)
QAVVTQESALTTSPGETVTLTCRSSTGAVTTHNYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #11
(SEQ ID NO: 586)
QAVVTQESALTTSPGETVTLTCRSSTGAVTTSHYANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #12
(SEQ ID NO: 587)
QAVVTQESALTTSPGETVTLTCRSSTGAVTTSNHANWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #13
(SEQ ID NO: 588)
QAVVTQESALTTSPGETVTLTCRSSTGAVTTSNYHNWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #14
(SEQ ID NO: 589)
QAVVTQESALTTSPGETVTLTCRSSTGAVTTSNYAHWIQEKPDHLFTGLIGGTNNRAPGVPARFS
GSLIGDKAALTITGAQTEDEAIYFCGLWYSNHLVFGGGTKLTVL
>Light Chain of SC16.67 IgG histidine scanning variant #15
(SEQ ID NO: 590)
QAVVTQESALTTSPGETVTLTCRSSTGAVTTSNYANWIQEKPDHLFTGLIGHTNNRAPGVPARFS
GSLIGDKAALTITGAQTEDEAIYFCGLWYSNHLVFGGGTKLTVL
>Light Chain of SC16.67 IgG histidine scanning variant #16
(SEQ ID NO: 591)
QAVVTQESALTTSPGETVTLTCRSSTGAVTTSNYANWIQEKPDHLFTGLIGGHNNRAPGVPARFS
GSLIGDKAALTITGAQTEDEAIYFCGLWYSNHLVFGGGTKLTVL
>Light Chain of SC16.67 IgG histidine scanning variant #17
(SEQ ID NO: 592)
QAVVTQESALTTSPGETVTLTCRSSTGAVTTSNYANWIQEKPDHLFTGLIGGTHNRAPGVPARFS
GSLIGDKAALTITGAQTEDEAIYFCGLWYSNHLVFGGGTKLTVL
>Light Chain of SC16.67 IgG histidine scanning variant #18
(SEQ ID NO: 593)
QAVVTQESALTTSPGETVTLTCRSSTGAVTTSNYANWIQEKPDHLFTGLIGGTNHRAPGVPARFS
GSLIGDKAALTITGAQTEDEAIYFCGLWYSNHLVFGGGTKLTVL
>Light Chain of SC16.67 IgG histidine scanning variant #19
(SEQ ID NO: 594)
QAVVTQESALTTSPGETVTLTCRSSTGAVTTSNYANWIQEKPDHLFTGLIGGTNNHAPGVPARFS
GSLIGDKAALTITGAQTEDEAIYFCGLWYSNHLVFGGGTKLTVL
>Light Chain of SC16.67 IgG histidine scanning variant #20
(SEQ ID NO: 595)
QAVVTQESALTTSPGETVTLTCRSSTGAVTTSNYANWIQEKPDHLFTGLIGGTNNRHPGVPARFS
GSLIGDKAALTITGAQTEDEAIYFCGLWYSNHLVFGGGTKLTVL
>Light Chain of SC16.67 IgG histidine scanning variant #21
(SEQ ID NO: 596)
QAVVTQESALTTSPGETVTLTCRSSTGAVTTSNYANWIQEKPDHLFTGLIGGTNNRAHGVPARFS
GSLIGDKAALTITGAQTEDEAIYFCGLWYSNHLVFGGGTKLTVL
>Light Chain of SC16.67 IgG histidine scanning variant #22
(SEQ ID NO: 597)
QAVVTQESALTTSPGETVTLTCRSSTGAVTTSNYANWIQEKPDHLFTGLIGGTNNRAPGVPARFS
GSLIGDKAALTITGAQTEDEAIYFCHLWYSNHLVFGGGTKLTVL
>Light Chain of SC16.67 IgG histidine scanning variant #23
(SEQ ID NO: 598)
QAVVTQESALTTSPGETVTLTCRSSTGAVTTSNYANWIQEKPDHLFTGLIGGTNNRAPGVPARFS
GSLIGDKAALTITGAQTEDEAIYFCGHWYSNHLVFGGGTKLTVL
>Light Chain of SC16.67 IgG histidine scanning variant #24
(SEQ ID NO: 599)
QAVVTQESALTTSPGETVTLTCRSSTGAVTTSNYANWIQEKPDHLFTGLIGGTNNRAPGVPARFS
GSLIGDKAALTITGAQTEDEAIYFCGLHYSNHLVFGGGTKLTVL
>Light Chain of SC16.67 IgG histidine scanning variant #25
(SEQ ID NO: 600)
QAVVTQESALTTSPGETVTLTCRSSTGAVTTSNYANWIQEKPDHLFTGLIGGTNNRAPGVPARFS
GSLIGDKAALTITGAQTEDEAIYFCGLWHSNHLVFGGGTKLTVL
>Light Chain of SC16.67 IgG histidine scanning variant #26
(SEQ ID NO: 601)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYHNHLVFGGGTKLTVL
>Light Chain of SC16.67 IgG histidine scanning variant #27
(SEQ ID NO: 602)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYSHHLVFGGGTKLTVL
>Light Chain of SC16.67 IgG histidine scanning variant #28
(SEQ ID NO: 603)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYSNALVFGGGTKLTVL
>Light Chain of SC16.67 IgG histidine scanning variant #29
(SEQ ID NO: 604)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYSNHHVFGGGTKLTVL
>Light Chain of SC16.67 IgG histidine scanning variant #30
(SEQ ID NO: 605)
QAVVTQESALTTSPGETVTLTCRSSTGAVTTSNYANWIQEKPDHLFTGLIGGTNNRAPGVPARFS
GSLIGDKAALTITGAQTEDEAIYFCGLWYSNHLHFGGGTKLTVL
>Humani#ed VH variant #1
(SEQ ID NO: 606)
EVQLVESGGGLVQPGGSLRLSCAASGFNISYYYIGWVRRAPGKGEELVARIYPTNGYTRYADSV
KGRFTISADTSKNTAYLQMNSLRAEDTAVYYCARAYGDSDHYSEAIDYSGQGTLVTVSS
>Humani#ed VH variant #2
(SEQ ID NO: 607)
EVQLVESGGGLVQPGGSLRLSCAASGFNIYSESIGWVRRAPGKGEELVARIYPTNGYTRYADSVK
GRFTISADTSKNTAYLQMNSLRAEDTAVYYCARSYYSHYDGHYHYYGLDYSGQGTLVTVSS
>Humani#ed VH variant #3
(SEQ ID NO: 608)
EVQLVESGGGLVQPGGSLRLSCAASGFNISYYSIGWVRRAPGKGEELVARIYPTNGYTRYADSV
KGRFTISADTSKNTAYLQMNSLRAEDTAVYYCARYHHSEYSDYYGYYSEYGFDYSGQGTLVTV
RS
>Humani#ed VH variant #4
(SEQ ID NO: 609)
EVQLVESGGGLVQPGGSLRLSCAASGFNIYSYSIGWVRRAPGKGEELVARIYPTNGYTRYADSV
KGRFTISADTSKNTAYLQMNSLRAEDTAVYYCAREHYHGYYDGAHYYALDYSGQGTLVTVSS
>Humani#ed VH variant #5
(SEQ ID NO: 610)
EVQLVESGGGLVQPGGSLRLSCAASGFNIEYYSIGWVRRAPGKGEELVARIYPTNGYTRYADSV
KGRFTISADTSKNTAYLQMNSLRAEDTAVYYCAREYYSYHDEYEYDYALDYSGQGTLVTVSS
>Humani#ed VH variant #6
(SEQ ID NO: 611)
EVQLVESGGGLVQPGGSLRLSCAASGFNIEHYSIGWVRRAPGKGEELVARIYPTNGYTRYADSV
KGRFTISADTSKNTAYLQMNSLRAEDTAVYYCARHHYYGYHSGYHHYAIDYSGQGTLVTVSS
>Humani#ed VH variant #7
(SEQ ID NO: 612)
EVQLVESGGGLVQPGGSLRLSCAASGFNISSSDIGWVRRAPGKGEELVARIYPTNGYTRYADSVK
GRFTISADTSKNTAYLQMNSLRAEDTAVYYCARYYYYDDDGYYYEHAIDYSGQGTLVTVSS
>Humani#ed VH variant #8
(SEQ ID NO: 613)
EVQLVESGGGLVQPGGSLRLSCAASGFNIEYESIGWVRRAPGKGEELVARIYPTNGYTRYADSV
KGRFTISADTSKNTAYLQMNSLRAEDTAVYYCARSEYSHYSGSHHYYALDYSGQGTLVTVSS
>Humani#ed VH variant #9
(SEQ ID NO: 614)
EVQLVESGGGLVQPGGSLRLSCAASGFNISEHSIGWVRRAPGKGEELVARIYPTNGYTRYADSVK
GRFTISADTSKNTAYLQMNSLRAEDTAVYYCARSYYHGYSHGHHYYAIDYSGQGTLVTVSS
>Humani#ed VH variant #10
(SEQ ID NO: 615)
EVQLVESGGGLVQPGGSLRLSCAASGFNIHSYYIGWVRRAPGKGEELVARTYPTNGYTRYADSV
KGRFTISADTSKNTAYLQMNSLRAEDTAVYYCARGYYGHEYDYHYDYYGGGFDYSGQGTLVT
VSS
>Humani#ed VH variant #11
(SEQ ID NO: 616)
EVQLVESGGGLVQPGGSLRLSCAASGFNIHYEEIGWVRRAPGKGEELVARTYPTNGYTRYADSV
KGRFTISADTSKNTAYLQMNSLRAEDTAVYYCARYSYYYHYGGGMDYSGQGTLVTVSS
>Humani#ed VH variant #12
(SEQ ID NO: 617)
EVQLVESGGLVQPGGSLRLSCAASGFNIESSYIGWVRRAPGKGEELVARIYPTNGYTRYADSVKG
RFTISADTSKNTAYLQMNSLRAEDTAVYYCARYDEYDGYYYEAMDYSGQGTLVTVSS
>Humani#ed VH variant #13
(SEQ ID NO: 618)
EVQLVESGGGLVQPGGSLRLSCAASGFNISEESIGWVRRAPGKGEELVARTYPTNGYTRYADSVK
GRFTISADTSKNTAYLQMNSLRAEDTAVYYCARSYYAHYSAGYYYYALDYSGQGTLVTVSS
>Humani#ed VH variant #14
(SEQ ID NO: 619)
EVQLVESGGGLVQPGGSLRLSCAASGFNISHYSIGWVRHAPGKGEELVARTYPTNGYTRYADSV
KGRFTISADTSKNTAYLQMNSLRAEDTAVYYCARFIHYSHYDGHSHYYGLDYSGQGTLVTVSS
>Humani#ed VH variant #15
(SEQ ID NO: 620)
EVQLVESGGGLVQPGGSLRLSCAASGFNIYDHSIGWVRRAPGKGEELVARTYPTNGYTRYADSV
KGRFTISADTSKNTAYLQMNSLRAEDTAVYYCARSSYSGYYGSFIHYYALDYSGQGTLVTVSS
>Trastuzumab (4D5) Light Chain
(SEQ ID NO: 621)
DIQMTQSPSSLSASVGDRVTITCRASQDVNTAVAWYQQKPGKAPKWYSASFLYSGVPSRFSGS
RSGTDFTLTISSLQPEDFATYYCQQHYTTPPTFGQGTKVEIK
>Heavy Chain CDR1 of SC16.4 IgG
(SEQ ID NO: 622)
GYTFTDYSMH
>Heavy Chain CDR2 of SC16.4 IgG
(SEQ ID NO: 623)
WINTETGEPGYADDFKG
>Heavy Chain CDR3 of SC16.4 IgG
(SEQ ID NO: 624)
ARYDGYAMDY
>Light Chain CDR1 of SC16.4 IgG
(SEQ ID NO: 625)
RASQDISNYLN
>Light Chain CDR2 of SC16.4 IgG
(SEQ ID NO: 626)
YTSRLHS
>Light Chain CDR3 of SC16.4 IgG
(SEQ ID NO: 627)
QQGDMLPWT
>Heavy Chain CDR1 of hSC16.13 IgG
(SEQ ID NO: 628)
GFSLSTSGMGVG
>Heavy Chain CDR2 of hSC16.13 IgG
(SEQ ID NO: 629)
HIWWDDVKRYSPSLKS
>Heavy Chain CDR3 of hSC16.13 IgG
(SEQ ID NO: 630)
ARIVSFDNDVVSAMDY
>Light Chain CDR1 of hSC16.13 IgG
(SEQ ID NO: 631)
SASSSVSYMY
>Light Chain CDR2 of hSC16.13 IgG
(SEQ ID NO: 632)
LTSNLAS
>Light Chain CDR3 of hSC16.13 IgG
(SEQ ID NO: 633)
QQWRSNPFT
>Heavy Chain CDR1 of hSC16.15 IgG
(SEQ ID NO: 634)
KASGYTFTRYWIH
>Heavy Chain CDR2 of hSC16.15 IgG
(SEQ ID NO: 635)
YINPTTVYTEFNQNFKD
>Heavy Chain CDR3 of hSC16.15 IgG
(SEQ ID NO: 636)
ARGGSNFFDY
>Light Chain CDR1 of hSC16.15 IgG
(SEQ ID NO: 637)
ENIYYNLA
>Light Chain CDR2 of hSC16.15 IgG
(SEQ ID NO: 638)
TANSLED
>Light Chain CDR3 of hSC16.15 IgG
(SEQ ID NO: 639)
KQAYDVPPT
>Heavy Chain CDR1 of hSC16.25 IgG
(SEQ ID NO: 640)
GFSLSTSGMGVG
>Heavy Chain CDR2 of hSC16.25 IgG
(SEQ ID NO: 641)
DIWWDDNKYYNPSLKS
>Heavy Chain CDR3 of hSC16.25 IgG
(SEQ ID NO: 642)
ARRVNYYYDPYYAMDY
>Light Chain CDR1 of hSC16.25 IgG
(SEQ ID NO: 643)
SSVSYMH
>Light Chain CDR2 of hSC16.25 IgG
(SEQ ID NO: 644)
DSSKLAS
>Light Chain CDR3 of hSC16.25 IgG
(SEQ ID NO: 645)
QQWSSNPLT
>Heavy Chain CDR1 of hSC16.34 IgG
(SEQ ID NO: 646)
KASGYTFTNYGMN
>Heavy Chain CDR2 of hSC16.34 IgG
(SEQ ID NO: 647)
WINTYTGDPTYADDFKG
>Heavy Chain CDR3 of hSC16.34 IgG
(SEQ ID NO: 648)
ARIGGNSPSDY
>Light Chain CDR1 of hSC16.34 IgG
(SEQ ID NO: 649)
QSVSNDVA
>Light Chain CDR2 of hSC16.34 IgG
(SEQ ID NO: 650)
YASNRYS
>Light Chain CDR3 of hSC16.34 IgG
(SEQ ID NO: 651)
QQDYSSPWT
>Heavy Chain CDR1 of SC16.67 IgG
(SEQ ID NO: 652)
AFTFTTYAMN
>Heavy Chain CDR2 of SC16.67 IgG
(SEQ ID NO: 653)
RIRNKSNNYATYYADSVKD
>Heavy Chain CDR3 of SC16.67 IgG
(SEQ ID NO: 654)
VFYYDYVY
>Light Chain CDR1 of SC16.67 IgG
(SEQ ID NO: 655)
RSSTGAVTTSNYAN
>Light Chain CDR2 of SC16.67 IgG
(SEQ ID NO: 656)
GTNNRAP
>Light Chain CDR3 of SC16.67 IgG
(SEQ ID NO: 657)
GLWYSNHLV

1. A pharmaceutical composition comprising an effective amount of an antigen-binding protein construct (ABPC) comprising:
a first antigen-binding domain that is capable of specifically binding DLL3 or an epitope of DLL3 presented on the surface of a target mammalian cell,
wherein:
(a) the dissociation rate of the first antigen-binding domain at a pH of about 4.0 to about 6.5 is faster than the dissociation rate at a pH of about 7.0 to about 8.0; or
(b) the dissociation constant (KD) of the first antigen-binding domain at a pH of about 4.0 to about 6.5 is greater than the KD at a pH of about 7.0 to about 8.0.

2. The pharmaceutical composition of claim 1, wherein the ABPC is degraded in the target mammalian cell following internalization of the ABPC by the target mammalian cell.

3. The pharmaceutical composition of claim 1, wherein the ABPC further comprises a conjugated toxin, radioisotope, drug, or small molecule.

4. A pharmaceutical composition comprising an effective amount of an antigen-binding protein construct (ABPC) comprising:
a first antigen-binding domain that is capable of specifically binding DLL3 or an epitope of DLL3 presented on the surface of a target mammalian cell; and
a conjugated toxin, radioisotope, drug, or small molecule,
wherein:
(a) the dissociation rate of the first antigen-binding domain at a pH of about 4.0 to about 6.5 is faster than the dissociation rate at a pH of about 7.0 to about 8.0; or
the dissociation constant (KD) of the first antigen-binding domain at a pH of about 4.0 to about 6.5 is greater than the KD at a pH of about 7.0 to about 8.0; and
(b) the composition provides for one or more of:
an increase in toxin liberation in the target mammalian cell as compared to a composition comprising the same amount of a control ABPC;
an increase in target mammalian cell killing as compared to a composition comprising the same amount of a control ABPC; and
an increase in endolysosomal delivery in the target mammalian cell as compared to a composition comprising the same amount of a control ABPC.

5. The pharmaceutical composition of claim 1, wherein the first antigen-binding domain comprises one of (a) though (g):
(a) a heavy chain variable domain of rovalpituzumab with one or more amino acids substituted with a histidine, wherein the heavy chain variable domain of rovalpituzumab comprises SEQ ID NO: 1; and/or
a light chain variable domain of rovalpituzumab with one or more amino acids substituted with a histidine, wherein the light chain variable domain of rovalpituzumab comprises SEQ ID NO: 2;
(b) a heavy chain variable domain of SC16.4 with one or more amino acids substituted with a histidine, wherein the heavy chain variable domain of SC16.4 comprises SEQ ID NO: 119; and/or
a light chain variable domain of SC16.4 with one or more amino acids substituted with a histidine, wherein the light chain variable domain of SC16.4 comprises SEQ ID NO: 120;
(c) a heavy chain variable domain of SC16.13 with one or more amino acids substituted with a histidine, wherein the heavy chain variable domain of SC16.13 comprises SEQ ID NO: 236; and/or
a light chain variable domain of SC16.13 with one or more amino acids substituted with a histidine, wherein the light chain variable domain of SC16.13 comprises SEQ ID NO: 237;
(d) a heavy chain variable domain of SC16.15 with one or more amino acids substituted with a histidine, wherein the heavy chain variable domain of SC16.15 comprises SEQ ID NO: 335; and/or
a light chain variable domain of SC16.15 with one or more amino acids substituted with a histidine, wherein the light chain variable domain of SC16.15 comprises SEQ ID NO: 336;
(e) a heavy chain variable domain of SC16.25 with one or more amino acids substituted with a histidine, wherein the heavy chain variable domain of SC16.25 comprises SEQ ID NO: 401; and/or
a light chain variable domain of SC16.25 with one or more amino acids substituted with a histidine, wherein the light chain variable domain of SC16.25 comprises SEQ ID NO: 402;
(f) a heavy chain variable domain of SC16.34 with one or more amino acids substituted with a histidine, wherein the heavy chain variable domain of SC16.34 comprises SEQ ID NO: 470; and/or
a light chain variable domain of SC16.34 with one or more amino acids substituted with a histidine, wherein the light chain variable domain of SC16.34 comprises SEQ ID NO: 471; and
(g) a heavy chain variable domain of SC16.67 with one or more amino acids substituted with a histidine, wherein the heavy chain variable domain of SC16.67 comprises SEQ ID NO: 537; and/or
a light chain variable domain of SC16.67 with one or more amino acids substituted with a histidine, wherein the light chain variable domain of SC16.67 comprises SEQ ID NO: 538.

6. The pharmaceutical composition of claim 1, wherein the first DLL3-binding domain comprises one of (a) through (g):
(a) a heavy chain variable domain comprising a CDR1, a CDR2, and a CDR3 of SEQ ID NOs: 3-5, respectively, with collectively a total of one or more amino acid positions in SEQ ID NOs: 3-5 substituted with a histidine; and/or
a light chain variable domain comprising a CDR1, a CDR2, and a CDR3 of SEQ ID NOs: 6-8, respectively, with collectively a total of one or more amino acid positions in SEQ ID NOs: 6-8 substituted with a histidine;
(b) a heavy chain variable domain comprising a CDR1, a CDR2, and a CDR3 of SEQ ID NOs: 622-624, respectively, with collectively a total of one or more amino acid positions in SEQ ID NOs: 622-624 substituted with a histidine; and/or
a light chain variable domain comprising a CDR1, a CDR2, and a CDR3 of SEQ ID NOs: 625-627, respectively, with collectively a total of one or more amino acid positions in SEQ ID NOs: 625-627 substituted with a histidine;
(c) a heavy chain variable domain comprising a CDR1, a CDR2, and a CDR3 of SEQ ID NOs: 628-630, respectively, with collectively a total of one or more amino acid positions in SEQ ID NOs: 628-630 substituted with a histidine; and/or
a light chain variable domain comprising a CDR1, a CDR2, and a CDR3 of SEQ ID NOs: 631-633, respectively, with collectively a total of one or more amino acid positions in SEQ ID NOs: 631-633 substituted with a histidine;
(d) a heavy chain variable domain comprising a CDR1, a CDR2, and a CDR3 of SEQ ID NOs: 634-636, respectively, with collectively a total of one or more amino acid positions in SEQ ID NOs: 634-636 substituted with a histidine; and/or
a light chain variable domain comprising a CDR1, a CDR2, and a CDR3 of SEQ ID NOs: 637-639, respectively, with collectively a total of one or more amino acid positions in SEQ ID NOs: 637-639 substituted with a histidine;
(e) a heavy chain variable domain comprising a CDR1, a CDR2, and a CDR3 of SEQ ID NOs: 640-642, respectively, with collectively a total of one or more amino acid positions in SEQ ID NOs: 640-642 substituted with a histidine; and/or
a light chain variable domain comprising a CDR1, a CDR2, and a CDR3 of SEQ ID NOs: 643-645, respectively, with collectively a total of one or more amino acid positions in SEQ ID NOs: 643-645 substituted with a histidine;
(f) a heavy chain variable domain comprising a CDR1, a CDR2, and a CDR3 of SEQ ID NOs: 646-648, respectively, with collectively a total of one or more amino acid positions in SEQ ID NOs: 646-648 substituted with a histidine; and/or
a light chain variable domain comprising a CDR1, a CDR2, and a CDR3 of SEQ ID NOs: 649-651, respectively, with collectively a total of one or more amino acid positions in SEQ ID NOs: 649-651 substituted with a histidine; and
(g) a heavy chain variable domain comprising a CDR1, a CDR2, and a CDR3 of SEQ ID NOs: 652-654, respectively, with collectively a total of one or more amino acid positions in SEQ ID NOs: 652-654 substituted with a histidine; and/or
a light chain variable domain comprising a CDR1, a CDR2, and a CDR3 of SEQ ID NOs: 655-657, respectively, with collectively a total of one or more amino acid positions in SEQ ID NOs: 655-657 substituted with a histidine.

7. The pharmaceutical composition of claim 1, wherein the first antigen-binding domain comprises one of (a) through (g):
(a) a heavy chain variable domain that is at least 90% identical to SEQ ID NO: 1, wherein the heavy chain variable domain includes a histidine at one or more positions in SEQ ID NO: 1 selected from the group consisting of: 27, 29, 31, 32, 34, 35, 50, 53, 54, 55, 58, 97, 98, 101, 103, 105, and 106; and/or
a light chain variable domain that is at least 90% identical to SEQ ID NO: 2, wherein the light chain variable domain includes a histidine at one or more positions in SEQ ID NO: 2 selected from the group consisting of: 25, 26, 29, 32, 33, 34, 51, 54, 89, 90, 93, 94, 95, and 96;
(b) a heavy chain variable domain that is at least 90% identical to SEQ ID NO: 119, wherein the heavy chain variable domain includes a histidine at one or more positions in SEQ ID NO: 119 selected from the group consisting of: 32, 50, 52, 53, 57, 58, 59, 60, 62, 64, 65, 97, 98, 99, 100, 102, 104, and 105; and/or
a light chain variable domain that is at least 90% identical to SEQ ID NO: 120, wherein the light chain variable domain includes a histidine at one or more positions in SEQ ID NO: 120 selected from the group consisting of: 29, 32, 34, 91, 92, 93, 94 and 96;
(c) a heavy chain variable domain that is at least 90% identical to SEQ ID NO: 236, wherein the heavy chain variable domain includes a histidine at one or more positions in SEQ ID NO: 236 selected from the group consisting of: 27, 29, 31, 33, 34, 35, 36, 37, 54, 55, 56, 58, 60, 98, 100, 102, 106, 108, and 110; and/or
a light chain variable domain that is at least 90% identical to SEQ ID NO: 237, wherein the light chain variable domain includes a histidine at one or more positions in SEQ ID NO: 237 selected from the group consisting of 25, 29, 32, 33, 49, 50, 90, 91, 93, and 95;
(d) a heavy chain variable domain that is at least 90% identical to SEQ ID NO: 335, wherein the heavy chain variable domain includes a histidine at one or more positions in SEQ ID NO: 335 selected from the group consisting of: 27, 32, 33, 34, 50, 98, 101, 102, 103, 104, and 105; and/or
a light chain variable domain that is at least 90% identical to SEQ ID NO: 336, wherein the light chain variable domain includes a histidine at one or more positions in SEQ ID NO: 336 selected from the group consisting of: 27, 29, 32, 34, 52, 53, 89, 90, 91, 92, 93, 94, and 96;
(e) a heavy chain variable domain that is at least 90% identical to SEQ ID NO: 401, wherein the heavy chain variable domain includes a histidine at one or more positions in SEQ ID NO: 401 selected from the group consisting of: 27, 101, 103, 104, 108, and 109; and/or
a light chain variable domain that is at least 90% identical to SEQ ID NO: 402, wherein the light chain variable domain includes a histidine at one or more positions in SEQ ID NO: 402 selected from the group consisting of: 30 and 31;
(f) a heavy chain variable domain that is at least 90% identical to SEQ ID NO: 470, wherein the heavy chain variable domain includes a histidine at one or more positions in SEQ ID NO: 470 selected from the group consisting of 24, 27, 29, 32, 34, 35, 50, 51, 53, 54, 56, 58, 59, 60, 63, 98, 101, 103, 105, 106, and 107; and/or
a light chain variable domain that is at least 90% identical to SEQ ID NO: 471, wherein the light chain variable domain includes a histidine at one or more positions in SEQ ID NO: 471 selected from the group consisting of: 29, 32, 33, 34, 50, 51, 53, 55, 89, 92, 94, 96 and 97; and
(g) a heavy chain variable domain that is at least 90% identical to SEQ ID NO: 537, wherein the heavy chain variable domain includes a histidine at one or more positions in SEQ ID NO: 537 selected from the group consisting of: 26, 29, 31, 32, 53, 54, 57, 58, 59, 65, 67, 68, 103, 104, and 106; and/or
a light chain variable domain that is at least 90% identical to SEQ ID NO: 538, wherein the light chain variable domain includes a histidine at one or more positions in SEQ ID NO: 538 selected from the group consisting of: 28, 34, 53, 93, 94, and 98.

8. The pharmaceutical composition of claim 1, wherein the first antigen-binding domain comprises one of (a) through (g):
(a) a light chain variable domain of SEQ ID NO: 2, SEQ ID NO: 55, SEQ ID NO: 56, SEQ ID NO: 59, SEQ ID NO: 62, SEQ ID NO: 63, SEQ ID NO: 64, SEQ ID NO: 65, SEQ ID NO: 66, SEQ ID NO: 69, SEQ ID NO: 72, SEQ ID NO: 73, SEQ ID NO: 76, SEQ ID NO: 77, SEQ ID NO: 78, SEQ ID NO: 79, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, or SEQ ID NO: 118, and/or
a heavy chain variable domain of SEQ ID NO: 1, SEQ ID NO: 17, SEQ ID NO: 19, SEQ ID NO: 21, SEQ ID NO: 22, SEQ ID NO: 24, SEQ ID NO: 25, SEQ ID NO: 26, SEQ ID NO: 29, SEQ ID NO: 30, SEQ ID NO: 31, SEQ ID NO: 34, SEQ ID NO: 43, SEQ ID NO: 44, SEQ ID NO: 47, SEQ ID NO: 49, SEQ ID NO: 51, SEQ ID NO: 52, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or SEQ ID NO: 98,
wherein the first antigen-binding domain does not comprise (i) a light chain variable domain of SEQ ID NO: 2 and a heavy chain variable domain of SEQ ID NO: 1; (ii) a light chain variable domain of SEQ ID NO: 2 and heavy chain variable domain that is not one of SEQ ID NOs: 17, 19, 21, 22, 24-26, 29-31, 34, 43, 44, 47, 49, 51, 52, and 81-98; or (iii) a heavy chain variable domain of SEQ ID NO: 1 and a light chain variable domain that is not one of SEQ ID NOs: 55, 56, 59, 62-66, 69, 72, 73, 76-79, and 99-118;
(b) a light chain variable domain of SEQ ID NO: 120, SEQ ID NO: 163, SEQ ID NO: 166, SEQ ID NO: 168, SEQ ID NO: 178, SEQ ID NO: 179, SEQ ID NO: 180, SEQ ID NO: 181, SEQ ID NO: 183, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, or SEQ ID NO: 235, and/or
a heavy chain variable domain of SEQ ID NO: 119, SEQ ID NO: 127, SEQ ID NO: 130, SEQ ID NO: 131, SEQ ID NO: 133, SEQ ID NO: 134, SEQ ID NO: 138, SEQ ID NO: 139, SEQ ID NO: 140, SEQ ID NO: 141, SEQ ID NO: 143, SEQ ID NO: 145, SEQ ID NO: 146, SEQ ID NO: 148, SEQ ID NO: 149, SEQ ID NO: 150, SEQ ID NO: 151, SEQ ID NO: 153, SEQ ID NO: 155, SEQ ID NO: 156; SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 216, SEQ ID NO: 217, SEQ ID NO: 218, SEQ ID NO: 220, SEQ ID NO: 221, or SEQ ID NO: 223,
wherein the first antigen-binding domain does not comprise (i) a light chain variable domain of SEQ ID NO: 120 and a heavy chain variable domain of SEQ ID NO: 119; (ii) a light chain variable domain of SEQ ID NO: 120 and heavy chain variable domain that is not one of SEQ ID NOs: 127, 130, 131, 133, 134, 138-141, 143, 145, 146, 148-151, 153, 155, 156, 185-214, 216-218, 220, 221, and 223; or (iii) a heavy chain variable domain of SEQ ID NO: 119 and a light chain variable domain that is not one of SEQ ID NOs: 163, 166, 168, 178-181, 183, and 226-235;
(c) a light chain variable domain of SEQ ID NO: 237, SEQ ID NO: 283, SEQ ID NO: 287, SEQ ID NO: 290, SEQ ID NO: 291, SEQ ID NO: 292, SEQ ID NO: 293, SEQ ID NO: 301, SEQ ID NO: 302, SEQ ID NO: 304, SEQ ID NO: 306, or SEQ ID NO: 334, and/or
a heavy chain variable domain of SEQ ID NO: 236, SEQ ID NO: 239, SEQ ID NO: 241, SEQ ID NO: 243, SEQ ID NO: 245, SEQ ID NO: 246, SEQ ID NO: 247, SEQ ID NO: 248, SEQ ID NO: 249, SEQ ID NO: 250, SEQ ID NO: 252, SEQ ID NO: 253, SEQ ID NO: 254, SEQ ID NO: 256, SEQ ID NO: 258, SEQ ID NO: 266, SEQ ID NO: 268, SEQ ID NO: 270, SEQ ID NO: 274, SEQ ID NO: 276, SEQ ID NO: 278, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 330, SEQ ID NO: 331, or SEQ ID NO: 332,
wherein the first antigen-binding domain does not comprise (i) a light chain variable domain of SEQ ID NO: 237 and a heavy chain variable domain of SEQ ID NO: 236; (ii) a light chain variable domain of SEQ ID NO: 237 and heavy chain variable domain that is not one of SEQ ID NOs: 239, 241, 243, 245-250, 252-254, 256, 258, 266, 268, 270, 274, 276, 278, 308-328, and 330-332; or (iii) a heavy chain variable domain of SEQ ID NO: 236 and a light chain variable domain that is not one of SEQ ID NOs: 283, 287, 290-293, 301, 302, 304, 306, and 334;
(d) a light chain variable domain of SEQ ID NO: 336, SEQ ID NO: 377, SEQ ID NO: 379, SEQ ID NO: 382, SEQ ID NO: 384, SEQ ID NO: 387, SEQ ID NO: 388, SEQ ID NO: 392, SEQ ID NO: 393, SEQ ID NO: 394, SEQ ID NO: 395, SEQ ID NO: 396, SEQ ID NO: 397, or SEQ ID NO: 399, and/or
a heavy chain variable domain of SEQ ID NO: 335, SEQ ID NO: 341, SEQ ID NO: 346, SEQ ID NO: 347, SEQ ID NO: 348, SEQ ID NO: 349, SEQ ID NO: 350, SEQ ID NO: 368, SEQ ID NO: 371, SEQ ID NO: 372, SEQ ID NO: 373, SEQ ID NO: 374, or SEQ ID NO: 375,
wherein the first antigen-binding domain does not comprise (i) a light chain variable domain of SEQ ID NO: 336 and a heavy chain variable domain of SEQ ID NO: 335; (ii) a light chain variable domain of SEQ ID NO: 336 and heavy chain variable domain that is not one of SEQ ID NOs: 341, 346-350, 368, and 371-375; or (iii) a heavy chain variable domain of SEQ ID NO: 335 and a light chain variable domain that is not one of SEQ ID NOs: 377, 379, 382, 384, 387, 388, 392-397, and 399;
(e) a light chain variable domain of SEQ ID NO: 402, SEQ ID NO: 450, or SEQ ID NO: 451, and/or
a heavy chain variable domain of SEQ ID NO: 401, SEQ ID NO: 404, SEQ ID NO: 434, SEQ ID NO: 436, SEQ ID NO: 437, SEQ ID NO: 441, or SEQ ID NO: 442,
wherein the first antigen-binding domain does not comprise (i) a light chain variable domain of SEQ ID NO: 402 and a heavy chain variable domain of SEQ ID NO: 401; (ii) a light chain variable domain of SEQ ID NO: 402 and heavy chain variable domain that is not one of SEQ ID NOs: 404, 434, 436, 437, 441, and 442; or (iii) a heavy chain variable domain of SEQ ID NO: 401 and a light chain variable domain that is not one of SEQ ID NO: 450 or 451;
(f) a light chain variable domain of SEQ ID NO: 471, SEQ ID NO: 515, SEQ ID NO: 518, SEQ ID NO: 519, SEQ ID NO: 520, SEQ ID NO: 521, SEQ ID NO: 522, SEQ ID NO: 524, SEQ ID NO: 526, SEQ ID NO: 528, SEQ ID NO: 531, SEQ ID NO: 533, SEQ ID NO: 535, or SEQ ID NO: 536, and/or
a heavy chain variable domain of SEQ ID NO: 470, SEQ ID NO: 473, SEQ ID NO: 476, SEQ ID NO: 478, SEQ ID NO: 481, SEQ ID NO: 483, SEQ ID NO: 484, SEQ ID NO: 485, SEQ ID NO: 486, SEQ ID NO: 488, SEQ ID NO: 489, SEQ ID NO: 491, SEQ ID NO: 493, SEQ ID NO: 494, SEQ ID NO: 495, SEQ ID NO: 498, SEQ ID NO: 503, SEQ ID NO: 506, SEQ ID NO: 508, SEQ ID NO: 510, SEQ ID NO: 511, or SEQ ID NO: 512,
wherein the first antigen-binding domain does not comprise (i) a light chain variable domain of SEQ ID NO: 471 and a heavy chain variable domain of SEQ ID NO: 470; (ii) a light chain variable domain of SEQ ID NO: 471 and heavy chain variable domain that is not one of SEQ ID NOs: 473, 476, 478, 481, 483-486, 488, 489, 491, 493-495, 498, 503, 506, 508, and 510-512; or (iii) a heavy chain variable domain of SEQ ID NO: 470 and a light chain variable domain that is not one of SEQ ID NOs: 515, 518-522, 524, 526, 528, 531, 533, 535, and 536; and
(g) a light chain variable domain of SEQ ID NO: 538, SEQ ID NO: 581, SEQ ID NO: 587, SEQ ID NO: 591, SEQ ID NO: 599, SEQ ID NO: 600, or SEQ ID NO: 604, and/or a heavy chain variable domain of SEQ ID NO: 537, SEQ ID NO: 539, SEQ ID NO: 542, SEQ ID NO: 544, SEQ ID NO: 545, SEQ ID NO: 552, SEQ ID NO: 553, SEQ ID NO: 556, SEQ ID NO: 557, SEQ ID NO: 558, SEQ ID NO: 564, SEQ ID NO: 566, SEQ ID NO: 567, SEQ ID NO: 572, SEQ ID NO: 573, or SEQ ID NO: 575,
wherein the first antigen-binding domain does not comprise (i) a light chain variable domain of SEQ ID NO: 538 and a heavy chain variable domain of SEQ ID NO: 537; (ii) a light chain variable domain of SEQ ID NO: 538 and heavy chain variable domain that is not one of SEQ ID NOs: 539, 542, 544, 545, 552, 553, 556-558, 564, 566, 567, 572, 573, and 575; or (iii) a heavy chain variable domain of SEQ ID NO: 537 and a light chain variable domain that is not one of SEQ ID NOs: 581, 587, 591, 599, 600, and 604.

9. The pharmaceutical composition of claim 1, wherein the composition provides for:
an increase in toxin liberation in the target mammalian cell as compared to a composition comprising the same amount of a control ABPC; and/or
an increase in target mammalian cell killing as compared to a composition comprising the same amount of a control ABPC.

10. The pharmaceutical composition of claim 1, wherein the composition provides for an increase in endolysosomal delivery in the target mammalian cell as compared to a composition comprising the same amount of a control ABPC.

11. The pharmaceutical composition of claim 1, wherein the composition:
results in a less of a reduction in the level of DLL3 presented on the surface of the target mammalian cell as compared to a composition comprising the same amount of a control ABPC; or
does not result in a detectable reduction in the level of DLL3 presented on the surface of the target mammalian cell.

12. The pharmaceutical composition of claim 1, wherein the target mammalian cell is a cancer cell.

13. The pharmaceutical composition of claim 1, wherein the ABPC is cytotoxic or cytostatic to the target mammalian cell.

14. (canceled)

15. The pharmaceutical composition of claim 1, wherein the ABPC comprises a single polypeptide.

16. The pharmaceutical composition of claim 15, wherein the antigen-binding domain is selected from the group consisting of: a VH domain, a VHH domain, a VNAR domain, and a scFv.

17. The pharmaceutical composition of claim 1, wherein the ABPC comprises two or more polypeptides.

18. The pharmaceutical composition of claim 17, wherein the ABPC is an antibody.

19. The pharmaceutical composition of claim 1, wherein the half-life of the ABPC in vivo is decreased as compared to the half-life of a control ABPC in vivo.

20. The pharmaceutical composition of claim 1, wherein the ABPC further comprises a second antigen-binding domain.

21. An antigen-binding protein construct (ABPC) comprising:
a first antigen-binding domain that is capable of specifically binding DLL3 or an epitope of DLL3 presented on the surface of a target mammalian cell,
wherein:
(a) the dissociation rate of the first antigen-binding domain at a pH of about 4.0 to about 6.5 is faster than the dissociation rate at a pH of about 7.0 to about 8.0; or
(b) the dissociation constant (KD) of the first antigen-binding domain at a pH of about 4.0 to about 6.5 is greater than the KD at a pH of about 7.0 to about 8.0.

22.-23. (canceled)

24. An antigen-binding protein construct (ABPC) comprising:
a first antigen-binding domain that is capable of specifically binding DLL3 or an epitope of DLL3 presented on the surface of a target mammalian cell; and
a conjugated toxin, radioisotope, drug, or small molecule,
wherein:
(a) the dissociation rate of the first antigen-binding domain at a pH of about 4.0 to about 6.5 is faster than the dissociation rate at a pH of about 7.0 to about 8.0; or
the dissociation constant (KD) of the first antigen-binding domain at a pH of about 4.0 to about 6.5 is greater than the KD at a pH of about 7.0 to about 8.0; and
(b) the composition provides for one or more of:
an increase in toxin liberation in the target mammalian cell as compared to a composition comprising the same amount of a control ABPC;
an increase in target mammalian cell killing as compared to a composition comprising the same amount of a control ABPC; and
an increase in endolysosomal delivery in the target mammalian cell as compared to a composition comprising the same amount of a control ABPC.

25.-40. (canceled)

41. A kit comprising at least one dose of the pharmaceutical composition of claim 1.

42. A method of treating a cancer characterized by having a population of cancer cells that have DLL3 or an epitope of DLL3 presented on their surface, the method comprising:
administering a therapeutically effective amount of the pharmaceutical composition of claim 1 to a subject identified as having a cancer characterized by having the population of cancer cells.

43. A method of reducing the volume of a tumor in a subject, wherein the tumor is characterized by having a population of cancer cells that have DLL3 or an epitope of DLL3 presented on their surface, the method comprising:
administering a therapeutically effective amount of the pharmaceutical composition of claim 1 to a subject identified as having a cancer characterized by having the population of cancer cells.

44. A method of inducing cell death in a cancer cell in a subject, wherein the cancer cell has DLL3 or an epitope of DLL3 presented on its surface, wherein the method comprises:
administering a therapeutically effective amount of the pharmaceutical composition of claim 1 to a subject identified as having a cancer characterized by having a population of the cancer cells.

45. A method of decreasing the risk of developing a metastasis or decreasing the risk of developing an additional metastasis in a subject having a cancer, wherein the cancer is characterized by having a population of cancer cells that have DLL3 or an epitope of DLL3 presented on their surface the method comprising:
administering a therapeutically effective amount of the pharmaceutical composition of claim 1 to a subject identified as having a cancer characterized by having the population of cancer cells.
